(19) United States
(12) Patent Application Publication
(10) Pub. No.: US 2026/0285857 A1
(43) Pub. Date: Sep. 24, 2026

(54) BICYCLIC AND HETEROCYCLIC AMIDE COMPOUNDS

(71) Applicants: Protego Biopharma, Inc., San Diego, CA (US); The Scripps Research Institute, La Jolla, CA (US)

(72) Inventors: Hank Michael James PETRASSI, Fallbrook, CA (US); Huang QIU, San Diego, CA (US); Bo QIN, La Jolla, CA (US); Richard F. LABAUDINIERE, San Diego, CA (US); Steven J. WILKENS, San Diego, CA (US); Sebastiaan Melvin Lambert van HOUT, San Diego, CA (US); Jeffery W. KELLY, La Jolla, CA (US); Nicholas Lok YAN, La Jolla, CA (US)

(21) Appl. No.: 19/666,855

(22) Filed: May 4, 2026

Related U.S. Application Data
(63) Continuation of application No. PCT/US2025/027325, filed on May 1, 2025.
(60) Provisional application No. 63/641,313, filed on May 1, 2024.

(51) Int. Cl. C07D 471/04; A61K 31/437; A61K 31/444; A61K 31/4545; A61K 31/496; A61K 31/4985; A61K 31/501; A61K 31/506; A61K 31/5377; A61K 31/5386; A61K 31/551; A61K 31/553; C07D 519/00

(52) U.S. Cl. CPC C07D 471/04; A61K 31/437; A61K 31/444; A61K 31/4545; A61K 31/496; A61K 31/4985; A61K 31/501; A61K 31/506; A61K 31/5377; A61K 31/5386; A61K 31/551; A61K 31/553; C07D 519/00

(57) ABSTRACT

Provided herein are compounds that stabilize immunoglobulin light chains, and pharmaceutically acceptable derivatives thereof. Also provided are pharmaceutical compositions containing the compounds and methods of using the compounds for treating a subject with light chain amyloidosis.

### CROSS-REFERENCE TO RELATED APPLICATIONS

This application is a continuation application of International Application No. PCT/US2025/027325, filed May 1, 2025, which claims priority to and the benefit of U.S. Provisional Application No. 63/641,313, filed on May 1, 2024, the contents of which are incorporated by reference herein in their entireties for all purposes.

### BACKGROUND

Light chain (LC) amyloidosis (AL amyloidosis) is a progressive and often fatal degenerative disease caused by monoclonal plasma cell proliferation, resulting in an abnormal free light chain (FLC) ratio and conformational changes within involved immunoglobulin light chains (iFLC) after secretion by clonal plasma cells that result in organ toxicity, e.g., cardiomyopathy, nephrotic syndrome and end-stage renal failure. Organ damage remains the major source of mortality and morbidity. Lambda light chains are more often amyloidogenic than the kappa light chains (~80%). The light chain conformational changes also often lead to light chain aggregation, which may also drive proteotoxicity in some post-mitotic tissues. The pathologic mechanisms of disease leading to organ toxicity include both toxicity of amyloidogenic LC and mass effects of deposits, both modulated by misfolded LC concentration.

Light chain amyloidosis patients are treated today by targeting the cancer component of this disease (proliferating clonal plasma cells) employing chemotherapy cocktails typically involving proteasome inhibitors (and, when possible, stem cell transplants), which ideally eliminate the clonal plasma cells secreting full-length light chains. However, complete clonal plasma cell eradication is achieved in only 30-40% of the patients and most eventually relapse. Restoration of organ function in treated patients is highly variable and often incomplete, resulting in poor outcomes. The current hematologic response criteria for AL amyloidosis define complete response (CR) as no evidence of monoclonal protein based on serum and urine immunofixation, as well as achieving a normal FLC ratio. The response criteria do not take the levels of iFLC into consideration. It has been shown that increased levels of iFLCs at the time of normal FLC ratio and complete or very good partial hematological response are associated with inferior incomes, i.e., lower organ response and lower overall survival. However, even low levels of amyloidogenic monoclonal FLC can result in organ dysfunction. Moreover, light chain amyloidosis patients exhibiting cardiac involvement are often too sick to tolerate chemotherapy and die within a year of diagnosis.

Thus, there is a need for additional treatments of light chain amyloidosis. The present disclosure addresses such need.

### SUMMARY

In some aspects, the present disclosure provides a compound of Formula (0):

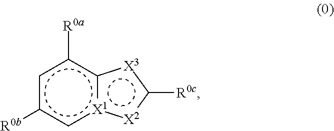

or a pharmaceutically acceptable salt thereof, wherein X¹, X², X³, R^{0a}, R^{0b} and R^{Oc} are as disclosed herein.

In some aspects, the present disclosure provides a compound of Formula (I):

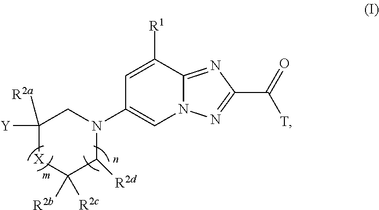

or a pharmaceutically acceptable salt thereof, wherein m, n, X, R¹, R^{2a}, R^{2b}, R^{2c}, R^{2d}, Y, and T are as disclosed herein.

In some aspects, the present disclosure provides a compound of Formula (I′):

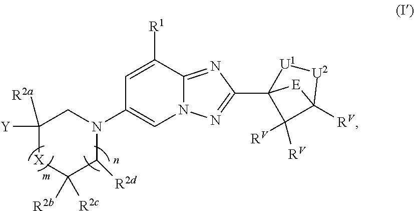

or a pharmaceutically acceptable salt thereof, wherein m, n, X, R¹, R^{2a}, R^{2b}, R^{2c}, R^{2d}, Y, E, U¹, U², and R^{V} are as disclosed herein.

In some aspects, the present disclosure provides a compound of Formula (I″-i) or (I″-ii):

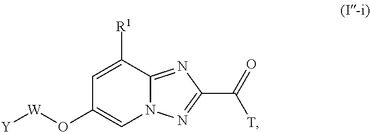

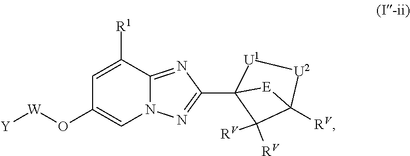

or a pharmaceutically acceptable salt thereof, wherein R¹, Y, T, W, E, U¹, U², and R^{V} are as disclosed herein.

In some aspects, the present disclosure provides a compound of Formula (I′″-i) or (I′″-ii):

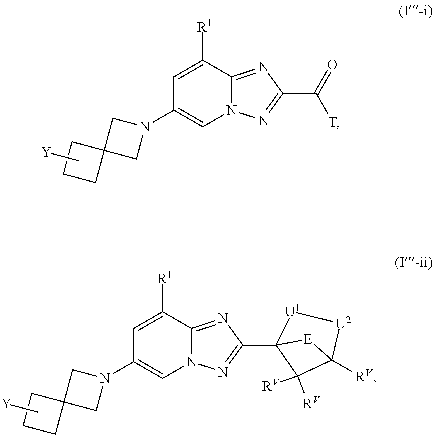

or a pharmaceutically acceptable salt thereof, wherein R¹, Y, T, E, U¹, U², and R^{V} are as disclosed herein.

In some aspects, the present disclosure provides a compound of Formula (VIII):

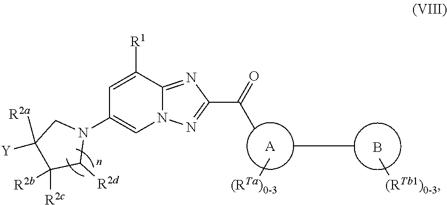

or a pharmaceutically acceptable salt thereof, wherein m, n, X, R¹, R^{2a}, R^{2b}, R^{2c}, R^{2d}, Y, R^{Ta}R^{Tb}, R^{Tb1}, Z, ring A, and ring B are as disclosed herein.

In some aspects, the present disclosure provides a compound selected from Tables 1-5, or a pharmaceutically acceptable salt thereof.

In some aspects, the present disclosure features pharmaceutical compositions comprising a compound described herein, and one or more pharmaceutically acceptable carriers or excipients.

In some aspects, the present disclosure provides a method of treating or preventing a disease (e.g., light chain amyloidosis) in a subject, comprising administering to the subject a compound or composition provided herein (e.g., in a therapeutically effective amount).

In some aspects, the present disclosure provides a compound or composition provided herein, for treating or preventing a disease (e.g., light chain amyloidosis) in a subject.

In some aspects, the present disclosure provides use of a compound provided herein in the manufacture of a medicament for treating or preventing a disease (e.g., light chain amyloidosis) in a subject.

In some embodiments, the disease is light chain amyloidosis.

In some aspects, the present disclosure provides a method of treating or preventing light chain amyloidosis in a subject, comprising administering to the subject a compound or composition provided herein (e.g., in a therapeutically effective amount).

In some aspects, the present disclosure provides a compound or composition provided herein, for treating or preventing light chain amyloidosis in a subject.

In some aspects, the present disclosure provides use of a compound provided herein in the manufacture of a medicament for treating or preventing light chain amyloidosis in a subject.

In some aspects, the present disclosure provides a method of stabilizing immunoglobulin light chains in a subject, comprising administering to the subject a compound or composition provided herein (e.g., in a therapeutically effective amount).

In some aspects, the present disclosure provides a compound or composition provided herein, for stabilizing immunoglobulin light chains in a subject.

In some aspects, the present disclosure provides use of a compound provided herein in the manufacture of a medicament for stabilizing immunoglobulin light chains in a subject.

In some aspects, the present disclosure provides a method of preventing or lessening immunoglobulin light chain misfolding and/or endoproteolysis in a subject, comprising administering to the subject a compound or composition provided herein (e.g., in a therapeutically effective amount).

In some aspects, the present disclosure provides a compound or composition provided herein, for preventing or lessening immunoglobulin light chain misfolding and/or endoproteolysis in a subject.

In some aspects, the present disclosure provides use of a compound provided herein in the manufacture of a medicament for preventing or lessening immunoglobulin light chain misfolding and/or endoproteolysis in a subject.

In some embodiments, the immunoglobulin light chains are stabilized in a native conformation thereof. In some embodiments, the immunoglobulin light chains are dimers.

In some aspects, the present disclosure provides a method of maintenance therapy upon recurrence of light chain amyloidosis following primary treatment by administering to a subject a compound or composition provided herein.

In some aspects, the present disclosure provides a compound or composition provided herein, for maintenance therapy upon recurrence of light chain amyloidosis following primary treatment.

In some aspects, the present disclosure provides use of a compound provided herein in the manufacture of a medicament for maintenance therapy upon recurrence of light chain amyloidosis following primary treatment.

In some aspects, the present disclosure provides a method of combination therapy using a compound or composition provided herein in combination with one or more additional active agents that treat light chain amyloidosis, deplete clonal plasma cells, stabilize immunoglobulin light chains, prevent or lessen immunoglobulin light chain misfolding and/or endoproteolysis, promote clearance of fibrils, or that are effective in maintenance therapy upon recurrence of light chain amyloidosis following primary treatment.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

### Compounds of the Present Disclosure

### Compounds of Formula (0)

In some aspects, the present disclosure provides a compound of Formula (0):

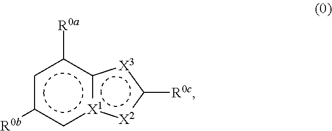

or a pharmaceutically acceptable salt thereof, wherein:
X¹ is N or C;
X² is N, O, or CH;
X³ is N, O, or CR^{0d};
R^{0d} is H or optionally substituted C₁-C₆ alkyl;
R^{0a} is halogen, cyano, —OH, —O(C₁-C₆ alkyl), —NH₂, —NH(C₁-C₆ alkyl), —N(C₁-C₆ alkyl)₂, or C₁-C₆ alkyl, wherein the —O(C₁-C₆ alkyl), —NH(C₁-C₆ alkyl), —N(C₁-C₆ alkyl)₂, or C₁-C₆ alkyl is optionally substituted;
R^{0b} is -L^{b1}-L^{b2};
L^{b1} is absent or —O(C₁-C₆ alkylene)-;
L^{b2} is 4- to 7-membered heterocyclyl, C₃-C₆ cycloalkyl, or C₁-C₆ alkyl, wherein the 4- to 7-membered heterocyclyl, C₃-C₆ cycloalkyl, or C₁-C₆ alkyl is optionally substituted;
R^{0c} is -L^{c1}-L^{c2};
L^{c1} is absent, —C(═O)—, —C(═O)—NH—, —C(═O)—NH—(C₁-C₆ alkylene)-, —C(═O)—N(C₁-C₆ alkyl)-, —C(═O)—O—, —C(═S)—, —S(═O)₂—, —S(═O)₂—NH—, —S(═O)₂—NH—(C₁-C₆ alkylene)-, or —S(═O)₂—O—; and
L^{c2} is H, C₁-C₆ alkyl, 4- to 12-membered heterocyclyl, or C₃-C₈ cycloalkyl, wherein the C₁-C₆ alkyl, 4- to 12-membered heterocyclyl, or C₃-C₈ cycloalkyl is optionally substituted.

### Compounds of Formula (I)

In some aspects, the present disclosure provides a compound of Formula (I):

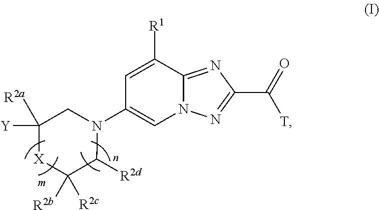

or a pharmaceutically acceptable salt thereof, wherein:
m is 0 or 1;
n is 0 or 1;
X is —CH₂— or —O—;
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, —NH₂, —NHC(═O)(C₁-C₆ alkyl), —NHS(═O)₂(C₁-C₆ alkyl), or —C(═O)NH₂;
R^{2a} is C₁-C₆ alkyl optionally substituted with one or more halogen, cyano, —OH, or —NH₂;
R^{2b} is H or C₁-C₆ alkyl, wherein the C₁-C₆ alkyl is optionally substituted with one or more halogen;
R^{2c} is H or C₁-C₆ alkyl, wherein the C₁-C₆ alkyl is optionally substituted with one or more halogen;
R^{2d} is H; or R^{2a} and R^{2d} together form C₁-C₆ alkylene optionally substituted with one or more halogen;
Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
T is —OR^{T1}, —N(R^{T1})₂, or —N(R^{T2})₂;
each R^{T1} independently is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta};
two R^{T2}, together with the atom to which they are attached, form 4- to 10-membered heterocyclyl optionally substituted with one or more R^{Ta};
each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb};
each R^{b} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{Tb} independently is oxo, halogen, cyano, —OH, —OR^{c}, —NH₂, —NHR^{c}, —N(R^{c})₂, —C(═O)—H, —C(═O)—R, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{c} independently is C₁-C₆ alkyl, C₂-C₆ alkenyl, or C₂-C₆ alkynyl; and
each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

It is understood that, for a compound of the present disclosure, variables m, n, X, R¹, R^{2a}, R^{2b}, R^{2c}, R^{2d}, Y, R^{Ya}, R^{Yb}, T, R^{T1}, R^{T2}, R^{Ta}, R^{Tb}, R^{Tc}, R^{b}, and R^{c} can each be, where applicable, selected from the groups described herein, and any group described herein for any of variables m, n, X, R¹, R^{2a}, R^{2b}, R^{2c}, R^{2d}, Y, R^{Ya}, R^{Yb}, T, R^{T1}, R^{T2}, R^{Ta}, R^{Tb}, R^{Tc}, R^{b}, and R^{c} can be combined, where applicable, with any group described herein for one or more of the remainder of variables m, n, X, R¹, R^{2a}, R^{2b}, R^{2c}, R^{2d}, Y, R^{Ya}, R^{Yb}, T, R^{T1}, R^{T2}, R^{Ta}, R^{Tb}, R^{Tc}, R^{b}, and R^{c}.

### Compounds of Formula (I)

In some aspects, the present disclosure provides a compound of Formula (I′):

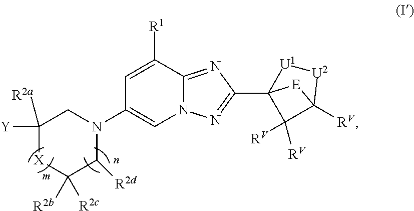

or a pharmaceutically acceptable salt thereof, wherein:
m is 0 or 1;
n is 0 or 1;
X is —CH₂— or —O—;
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, or —NH₂;
R^{2a} is C₁-C₆ alkyl optionally substituted with one or more halogen, cyano, —OH, or —NH₂;
R^{2b} is H or C₁-C₆ alkyl, wherein the C₁-C₆ alkyl is optionally substituted with one or more halogen;
R^{2c} is H or C₁-C₆ alkyl, wherein the C₁-C₆ alkyl is optionally substituted with one or more halogen;
R^{2d} is H; or R^{2a} and R^{2d} together form C₁-C₆ alkylene optionally substituted with one or more halogen;
Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
E is —(C₁-C₆ alkylene)- optionally substituted with one or more oxo, halogen, cyano, —OH, —O(C₁-C₆ alkyl), —NH₂, —NH(C₁-C₆ alkyl), or —N(C₁-C₆ alkyl)₂;
one of U¹ and U² is —O—, and the other one of U¹ and U² is —C(R^{U})₂—;
each R^{U} independently is H, halogen, cyano, —OH, —O(C₁-C₆ alkyl), —NH₂, —NH(C₁-C₆ alkyl), —N(C₁-C₆ alkyl)₂, or C₁-C₆ alkyl;
each R^{V} independently is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—R^{A}, —C(═O)—H, —C(═O)—R^{A}, —C(═O)—OH, —C(═O)—OR^{A}, —C(═O)—NH₂, —C(═O)—NHR^{A}, —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Va};
each R^{A} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{Va} independently is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{B} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{Vb} independently is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{C} independently is C₁-C₆ alkyl, C₂-C₆ alkenyl, or C₂-C₆ alkynyl; and
each R^{Vc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

It is understood that, for a compound of the present disclosure, variables m, n, X, R¹, R^{2a}, R^{2b}, R^{2c}, R^{2d}, Y, R^{Ya}, R^{Yb}, E, U¹, U², R^{U}, R^{V}, R^{Va}, R^{Vb}, R^{Vc}, R^{A}, R^{B}, and R^{C} can each be, where applicable, selected from the groups described herein, and any group described herein for any of variables m, n, X, R¹, R^{2a}, R^{2b}, R^{2c}, R^{2d}, Y, R^{Ya}, R^{Yb}, E, U, U², R^{U}, R^{V}, R^{Va}, R^{Vb}, R^{Vc}, R^{A}, R^{B}, and R^{C} can be combined, where applicable, with any group described herein for one or more of the remainder of variables m, n, X, R¹, R^{2a}, R^{2b}, R^{2c}, R^{2d}, Y, R^{Ya}, R^{Yb}, E, U¹, U², R^{U}, R^{V}, R^{Va}, R^{Vb}, R^{Vc}, R^{A}, R^{B}, and R^{C}.

### Compounds of Formula (I″-i) or Formula (I″-ii)

In some aspects, the present disclosure provides a compound of Formula (I″-i) or (I″-ii):

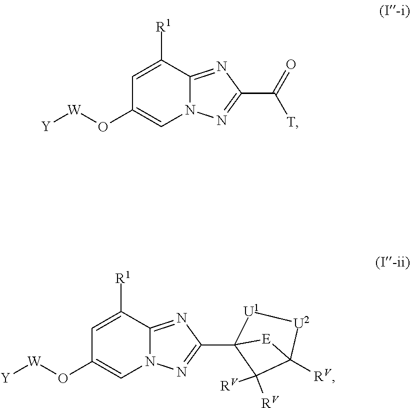

or a pharmaceutically acceptable salt thereof, wherein:
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, or —NH₂;
W is —(C₁-C₆ alkylene)- or —(C₃-C₆ cycloalkylene)-, wherein the —(C₁-C₆ alkylene)- or —(C₃-C₆ cycloalkylene)- is optionally substituted with one or more halogen, cyano, —OH, —O(C₁-C₆ alkyl), —NH₂, —NH(C₁-C₆ alkyl), —N(C₁-C₆ alkyl)₂, or C₁-C₆ alkyl;
Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
T is —OR^{T1}, —N(R^{T1})₂, or —N(R^{T2})₂;
each R^{T1} independently is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta};
two R^{T2}, together with the atom to which they are attached, form 4- to 10-membered heterocyclyl optionally substituted with one or more R^{Ta};
each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb};
each R^{b} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{Tb} independently is oxo, halogen, cyano, —OH, —OR^{c}, —NH₂, —NHR^{c}, —N(R^{c})₂, —C(═O)—H, —C(═O)—R, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{c} independently is C₁-C₆ alkyl, C₂-C₆ alkenyl, or C₂-C₆ alkynyl;
each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl;
E is —(C₁-C₆ alkylene)- optionally substituted with one or more oxo, halogen, cyano, —OH, —O(C₁-C₆ alkyl), —NH₂, —NH(C₁-C₆ alkyl), or —N(C₁-C₆ alkyl)₂; one of U¹ and U² is —O—, and the other one of U¹ and U² is —C(R^{U})₂—;
each R^{U} independently is H, halogen, cyano, —OH, —O(C₁-C₆ alkyl), —NH₂, —NH(C₁-C₆ alkyl), —N(C₁-C₆ alkyl)₂, or C₁-C₆ alkyl;
each R^{V} independently is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—R^{A}, —C(═O)—H, —C(═O)—R^{A}, —C(═O)—OH, —C(═O)—OR^{A}, —C(═O)—NH₂, —C(═O)—NHR^{A}, —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Va};
each R^{A} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{Va} independently is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{B} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{Vb} independently is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{C} independently is C₁-C₆ alkyl, C₂-C₆ alkenyl, or C₂-C₆ alkynyl; and
each R^{Vc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

It is understood that, for a compound of the present disclosure, variables W, Y, R^{Ya}R^{Yb}, T, R^{T1}, R^{T2}, R^{Ta}, R^{Tb}, R^{Tc}, R^{b}, R^{c}, E, U¹, U², R^{U}, R^{V}, R^{Va}, R^{Vb}, R^{Vc}, R^{A}, R^{B}, and R^{C} can each be, where applicable, selected from the groups described herein, and any group described herein for any of variables W, Y, R^{Y}a, R^{Yb}, T, R^{T1}, R^{T2}, R^{Ta}, R^{Tb}, R^{Tc}, E, U¹, U², R^{U}, R^{V}, R^{Va}. R^{Vb}, R^{Vc}, R^{A}, R^{B}, and R^{C} can be combined, where applicable, with any group described herein for one or more of the remainder of variables W, Y, R^{Y}a, R^{Yb}, T, R^{T1}, R^{T2}, R^{Ta}, R^{Tb}, R^{Tc}, E, U¹, U², R^{U}, R^{V}, R^{Va}, R^{Vb}, R^{Vc}, R^{A}, R^{B}, and R^{C}.

### Compounds of Formula (I′″-i) or Formula (I′″-ii)

In some aspects, the present disclosure provides a compound of Formula (I′″-i) or (I′″-ii):

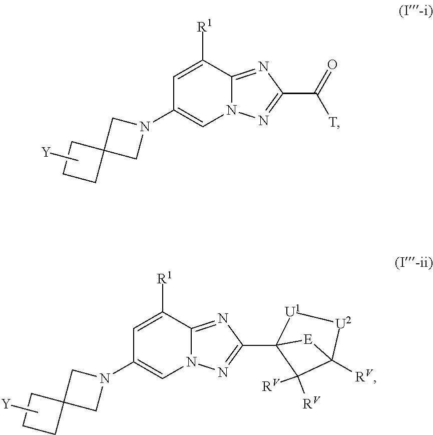

or a pharmaceutically acceptable salt thereof, wherein:
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, or —NH₂;
Y is C₃-C₈ cycloalkyl, C₆-C₁₀ aryl, or 5- to 10-membered heteroaryl, wherein the C₃-C₈cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
T is —OR^{T1}, —N(R^{T1})₂, or —N(R^{T2})₂;
each R^{T1} independently is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta};
two R^{T2}, together with the atom to which they are attached, form 4- to 10-membered heterocyclyl optionally substituted with one or more R^{Ta};
each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb};
each R^{b} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{Tb} independently is oxo, halogen, cyano, —OH, —OR^{c}, —NH₂, —NHR^{c}, —N(R^{c})₂, —C(═O)—H, —C(═O)—R, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc};
each R^{c} independently is C₁-C₆ alkyl, C₂-C₆ alkenyl, or C₂-C₆ alkynyl;
each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl;
E is —(C₁-C₆ alkylene)- optionally substituted with one or more oxo, halogen, cyano, —OH, —O(C₁-C₆ alkyl), —NH₂, —NH(C₁-C₆ alkyl), or —N(C₁-C₆ alkyl)₂;
one of U¹ and U² is —O—, and the other one of U¹ and U² is —C(R^{U})₂—;
each R^{U} independently is H, halogen, cyano, —OH, —O(C₁-C₆ alkyl), —NH₂, —NH(C₁-C₆ alkyl), —N(C₁-C₆ alkyl)₂, or C₁-C₆ alkyl;
each R^{V} independently is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—R^{A}, —C(═O)—H, —C(═O)—R^{A}, —C(═O)—OH, —C(═O)—OR^{A}, —C(═O)—NH₂, —C(═O)—NHR^{A}, —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Va};
each R^{A} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{Va} independently is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb};
each R^{B} independently is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{Vb} independently is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc};
each R^{C} independently is C₁-C₆ alkyl, C₂-C₆ alkenyl, or C₂-C₆ alkynyl;
each R^{Vc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

It is understood that, for a compound of the present disclosure, variables Y, R^{Ya}, R^{Yb}, T, R^{T1}, R^{T2}, R^{Ta}, R^{Tb}, R^{Tc}, R^{b}, R^{c}, E, U¹, U², R^{U}, R^{V}, R^{Va}, R^{Vb}, R^{Vc}, R^{A}, R^{B}, and R^{C} can each be, where applicable, selected from the groups described herein, and any group described herein for any of variables Y, R^{Ya}, R^{Yb}, T, R^{T1}, R^{T2}, R^{Ta}, R^{Tb}, R^{Tc}, R^{b}, R^{c}, E, U¹, U², R^{U}, R^{V}, R^{Va}, R^{Vb}, R^{Vc}, R^{A}, R^{B}, and R^{C} can be combined, where applicable, with any group described herein for one or more of the remainder of variables Y, R^{Y}a, R^{Yb}, T, R^{T1}, R^{T2}, R^{Ta}, R^{Tb}, R^{Tc}, R^{b}, R^{c}, E, U¹, U², R^{U}, R^{V}, R^{Va}, R^{Vb}, R^{Vc}, R^{A}, R^{B}, and R^{C}.

### Compounds of Formula (II), (II-a), (I-b), (III), (III-a), (III-b), (IV), (IV-a), (IV-b), (V), (V-a), (V-b), (VI), (VI-a), (VI-b), (VII), (VII-a), (VII-b), (VIII), (VIII-a), and (VIII-b)

In some aspects, the present disclosure provides a compound of Formula (II):

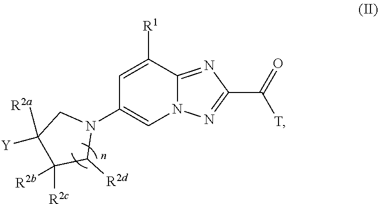

or a pharmaceutically acceptable salt thereof, wherein n, R¹, R^{2a}, R^{2b}, R^{2c}, R^{2d}, Y, and T are each as defined in Formula (I), or as described herein in any combination.

In some aspects, the present disclosure provides a compound of Formula (II-a) or (II-b):

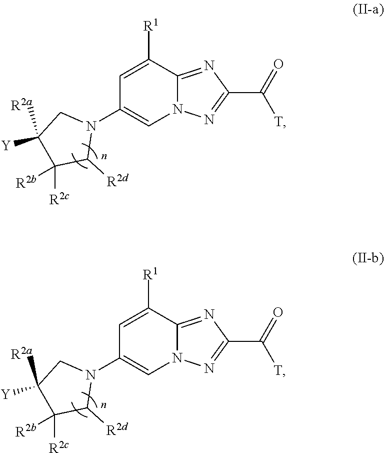

or a pharmaceutically acceptable salt thereof, wherein n, R¹, R^{2a}, R^{2b}, R^{2c}, R^{2d}, Y, and T are each as defined in Formula (I), or as described herein in any combination.

In some aspects, the present disclosure provides a compound of Formula (III):

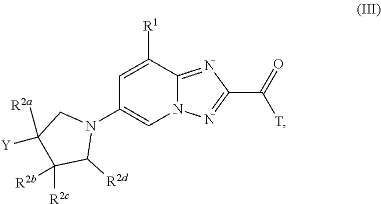

or a pharmaceutically acceptable salt thereof, wherein R¹, R^{2a}, R^{2b}, R^{2c}, R^{2d}, Y, and T are each as defined in Formula (I), or as described herein in any combination.

In some aspects, the present disclosure provides a compound of Formula (III-a) or (III-b):

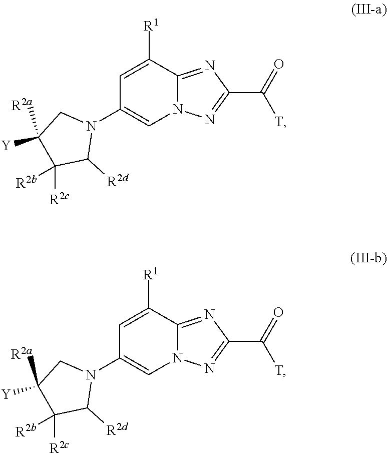

or a pharmaceutically acceptable salt thereof, wherein R¹, R^{2a}, R^{2b}, R^{2c}, R^{2d}, Y, and T are each as defined in Formula (I), or as described herein in any combination.

In some aspects, the present disclosure provides a compound of Formula (IV):

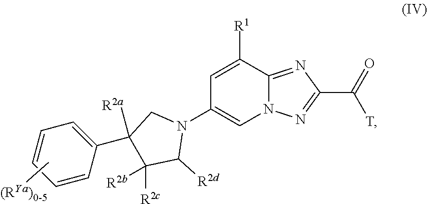

or a pharmaceutically acceptable salt thereof, wherein R¹, R^{2a}, R^{2b}, R^{2c}, R^{2d}, R^{Ya}, and T are each as defined in Formula (I), or as described herein in any combination.

In some aspects, the present disclosure provides a compound of Formula (IV-a) or Formula (IV-b):

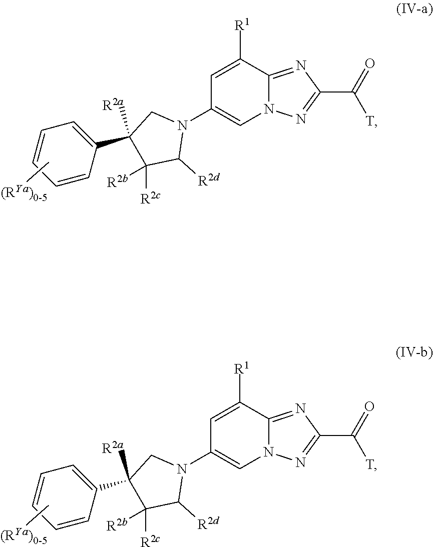

or a pharmaceutically acceptable salt thereof, wherein R¹, R^{2a}, R^{2b}, R^{2c}, R^{2d}, R^{Y}a, and T are each as defined in Formula (I), or as described herein in any combination.

In some aspects, the present disclosure provides a compound of Formula (V):

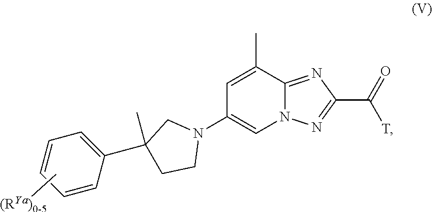

or a pharmaceutically acceptable salt thereof, wherein R^{Ya} and T are each as defined in Formula (I), or as described herein in any combination.

In some aspects, the present disclosure provides a compound of Formula (V-a) or Formula (V-b):

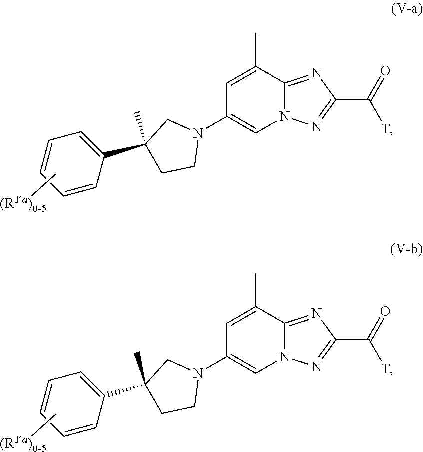

or a pharmaceutically acceptable salt thereof, wherein R^{Ya} and T are each as defined in Formula (I), or as described herein in any combination.

In some aspects, the present disclosure provides a compound of Formula (VI):

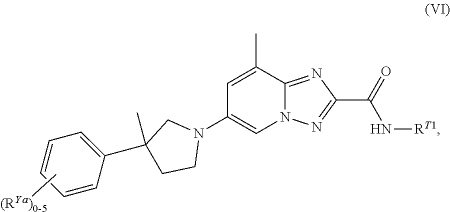

or a pharmaceutically acceptable salt thereof, wherein R^{Ya} and R^{T1} are each as defined in Formula (I), or as described herein in any combination.

In some aspects, the present disclosure provides a compound of Formula (VI-a) or Formula (VI-b):

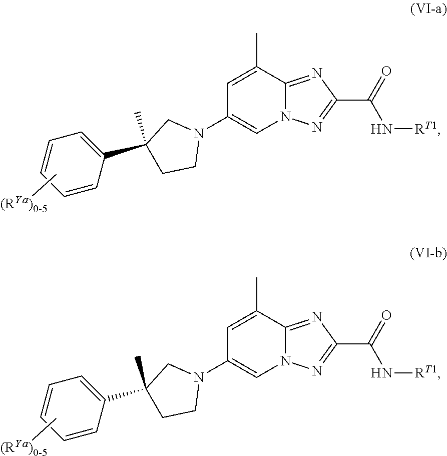

or a pharmaceutically acceptable salt thereof, wherein R^{Ya} and R^{T1} are each as defined in Formula (I), or as described herein in any combination.

In some aspects, the present disclosure provides a compound of Formula (VII):

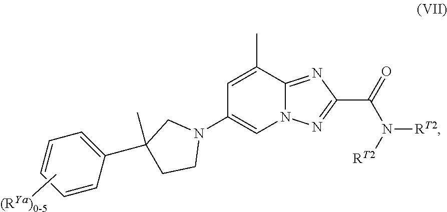

or a pharmaceutically acceptable salt thereof, wherein R^{Ya} and R^{T2} are each as defined in Formula (I), or as described herein in any combination.

In some aspects, the present disclosure provides a compound of Formula (VII-a) or Formula (VII-b):

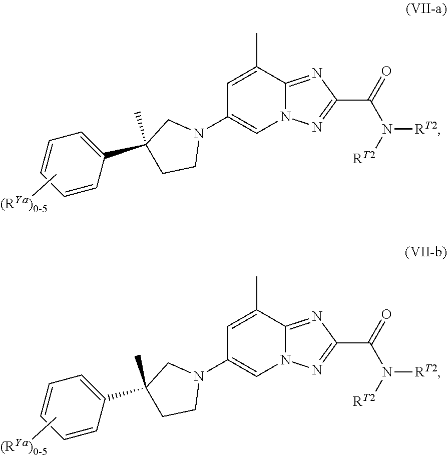

or a pharmaceutically acceptable salt thereof, wherein R^{Ya} and R^{T2} are each as defined in Formula (I), or as described herein in any combination.

In some aspects, the present disclosure provides a compound of Formula (VIII):

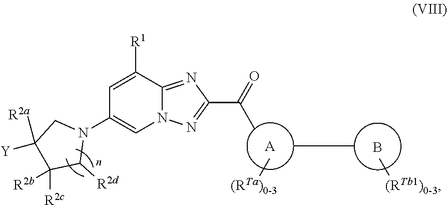

or a pharmaceutically acceptable salt thereof, wherein:
n is 0 or 1;
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, —NH₂, —NHC(═O)(C₁-C₆ alkyl), —NHS(═O)₂(C₁-C₆ alkyl), or —C(═O)NH₂;
R^{2a} is C₁-C₆ alkyl optionally substituted with one or more halogen, cyano, —OH, or —NH₂;
R^{2b} is H or C₁-C₆ alkyl, wherein the C₁-C₆ alkyl is optionally substituted with one or more halogen;
R^{2c} is H or C₁-C₆ alkyl, wherein the C₁-C₆ alkyl is optionally substituted with one or more halogen;
R^{2d} is H; or R^{2a} and R^{2d} together form C₁-C₆ alkylene optionally substituted with one or more halogen;
Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
ring A is

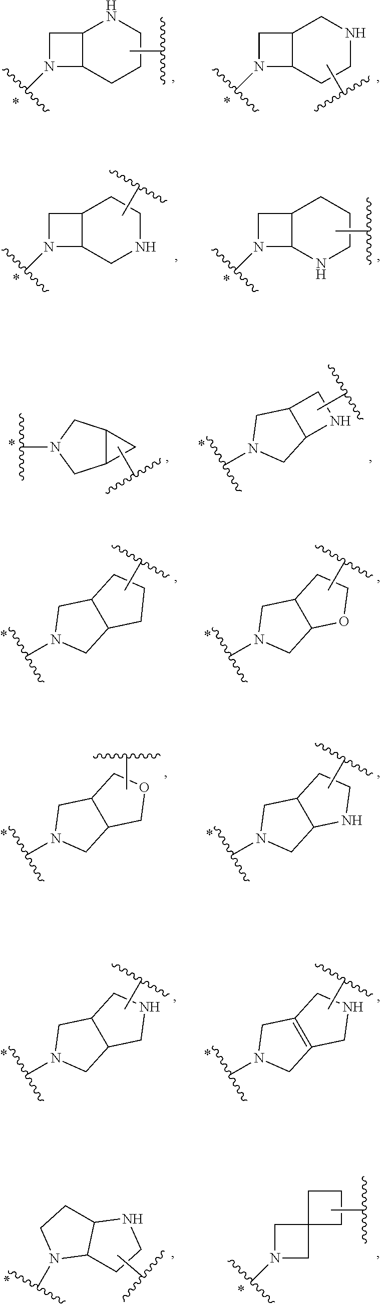

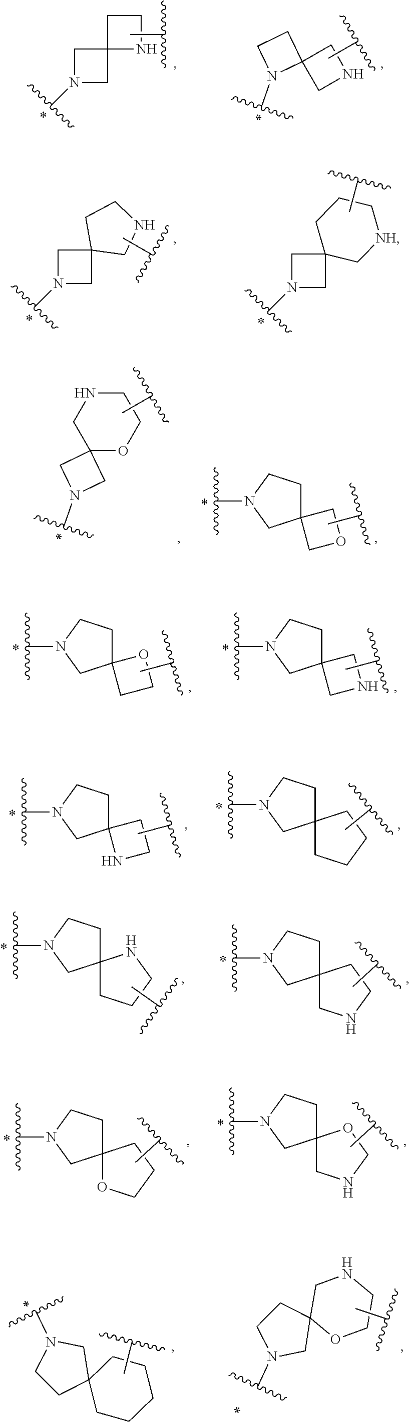

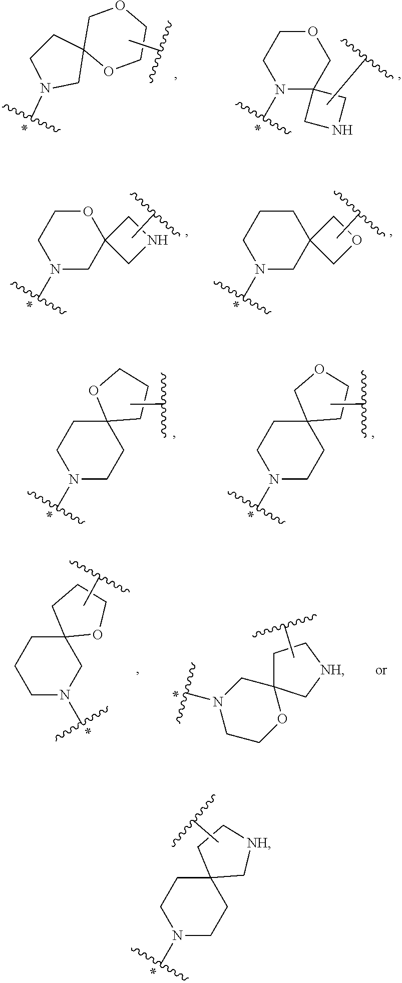

where * indicates connectivity to the carbonyl group at triazolopyridine (i.e., not the connectivity to Z);
B is absent (B is H) or ring B is C₃-C₆ cycloalkyl, 4- to 10-membered heterocyclyl, C₆-C₁₀ aryl, or 5- to 10-membered heteroaryl;
Z is absent, —CH₂—, —O—, or —C(═O)—;
each R^{Ta} independently is oxo, halogen, cyano, —OH, —OR^{Tb}, —NH₂, —NHR^{Tb}, —N(R^{Tb})₂, —C(═O)—H, —C(═O)—R^{Tb}, —C(═O)—OH, —C(═O)—OR^{Tb}, —C(═O)—NH₂, —C(═O)—NHR^{Tb}, —C(═O)—N(R^{Tb})₂, —S(═O)₂—R^{Tb}, —S(═O)(═NH)—R^{Tb}, C₁-C₆ alkyl, C₁-C₆ haloalkyl, C₂-C₆ alkenyl, C₂-C₆ haloalkenyl, C₂-C₆ alkynyl, or C₂-C₆ alkynyl;
each R^{Tb} is C₁-C₆ alkyl, C₁-C₆ haloalkyl, C₂-C₆ alkenyl, C₂-C₆ haloalkenyl, C₂-C₆ alkynyl, or C₂-C₆ alkynyl; and
each R^{Tb} is independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl;
wherein when B is absent (B is H), Z is absent.

As used herein, when a bicyclic ring is presented with a bond that is not fixed at a ring atom, such as in

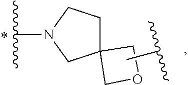

the bond can be formed at any available ring atom not limited to the particular ring which the line is drawn unless expressly stated. For example,

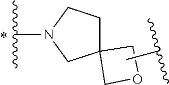

can be

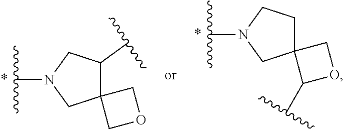 [SMILES: *C1CN(*)CC12COC2 ; *C1OCC12CCN(*)C2]

or other structures formed by placing the floating bond on the pyrrolidine ring or the oxetane ring.

In some aspects, the present disclosure provides a compound of Formula (VIII-a) or (VIII-b):

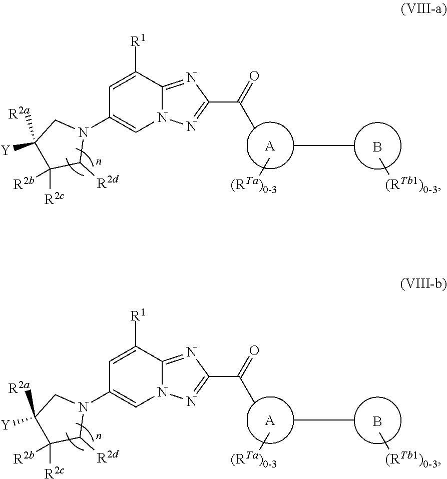

or a pharmaceutically acceptable salt thereof, wherein n, R¹, R^{2a}, R^{2b}, R^{2c}, R^{2d}, R^{Ta}, R^{Tb1}, Y, Z, ring A and ring B are each as defined in Formula (VIII), or as described herein in any combination.

It is understood that, for a compound of the present disclosure, variables m, n, X, R¹, R^{2a}, R^{2b}, R^{2c}, R^{2d}, Y, R^{Y}a, R^{Yb}, T, R^{T1}, R^{T2}, R^{Ta}, R^{Tb}, R^{Tb1}, R^{Tc}, Z, ring A, and ring B can each be, where applicable, selected from the groups described herein, and any group described herein for any of variables m, n, X, R¹, R^{2a}, R^{2b}, R^{2c}, R^{2d}, Y, R^{Ya}, R^{Yb}, T, R^{T1}, R^{T2}, R^{Ta}, R^{Tb1}, R^{Tc}, Z, ring A, and ring B can be combined, where applicable, with any group described herein for one or more of the remainder of variables m, n, X, R¹, R^{2a}, R^{2b}, R^{2c}, R^{2d}, Y, R^{Y}a, R^{Yb}, T, R^{T1}, R^{T2}, R^{Ta}, R^{Tb1}, R^{Tc}, Z, ring A, and ring B.

Compounds of Formula (II), (II′-a), (II′-b), (II′-c), (II′-d), (II′-e), (II′-f), (III′), (III′-a), (III′-b), (III′-c), (III′-d), (III′-e), (III′-f), (IV′), (IV′-a), (IV′-b), (IV′-c), (IV′-d), (IV′-e), (IV′-f), (V), ((V′-a), (V′-b), (V′-c), (V′-d), (V′-e), and (V′-f)

In some aspects, the present disclosure provides a compound of Formula (II′):

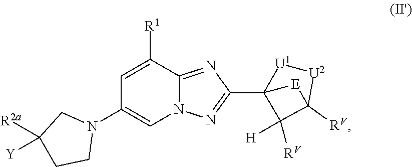

or a pharmaceutically acceptable salt thereof, wherein R¹, R^{2a}, Y, E, U¹, U², and R^{V} are each as defined in Formula (I′), or as described herein in any combination.

In some aspects, the present disclosure provides a compound of Formula (II′-a), (II′-b), (II′-c), (II′-d), (II′-e), or (II′-f):

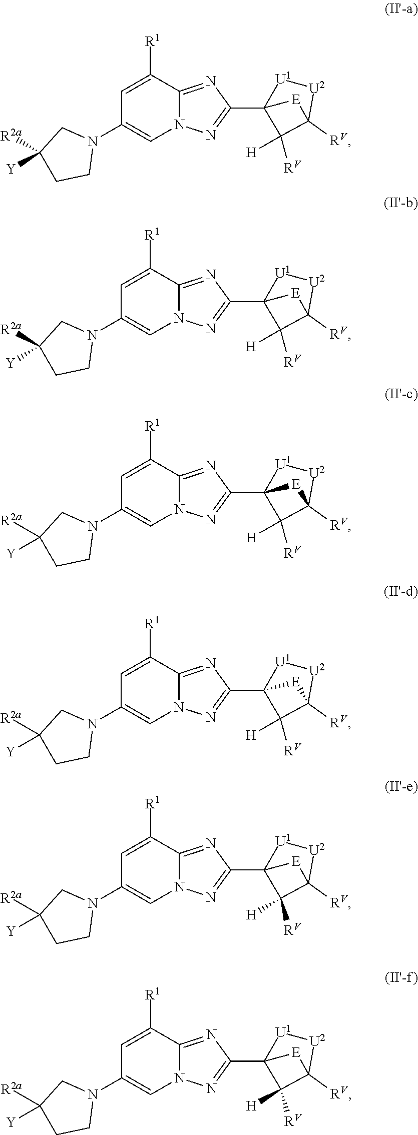

or a pharmaceutically acceptable salt thereof, wherein R¹, R^{2a}, Y, E, U¹, U², and R^{V} are each as defined in Formula (I′), or as described herein in any combination.

In some aspects, the present disclosure provides a compound of Formula (III′):

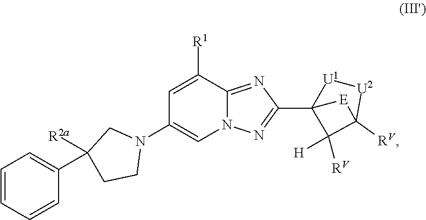

or a pharmaceutically acceptable salt thereof, wherein R¹, R^{2a}, E, U¹, U², and R^{V} are each as defined in Formula (I′), or as described herein in any combination.

In some aspects, the present disclosure provides a compound of Formula (III′-a), (III′-b), (III′-c), (III′-d), (III′-e), or (III′-f):

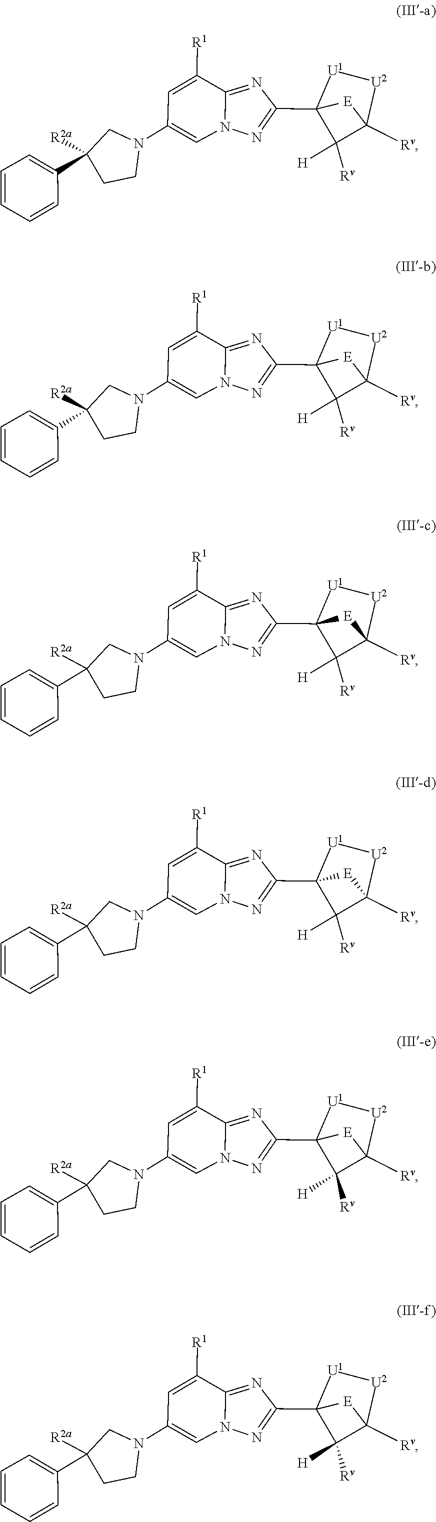

or a pharmaceutically acceptable salt thereof, wherein R¹, R^{2a}, E, U¹, U², and R^{V} are each as defined in Formula (I′), or as described herein in any combination.

In some aspects, the present disclosure provides a compound of Formula (IV′):

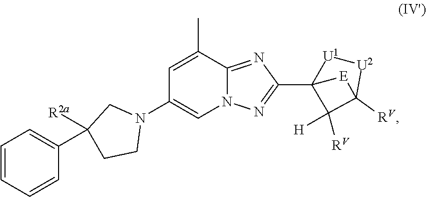

or a pharmaceutically acceptable salt thereof, wherein R^{2a}, E, U¹, U², and R^{V} are each as defined in Formula (I′), or as described herein in any combination.

In some aspects, the present disclosure provides a compound of Formula (IV′-a), (IV′-b), (IV′-c), (IV′-d), (IV′-e), or (IV′-f):

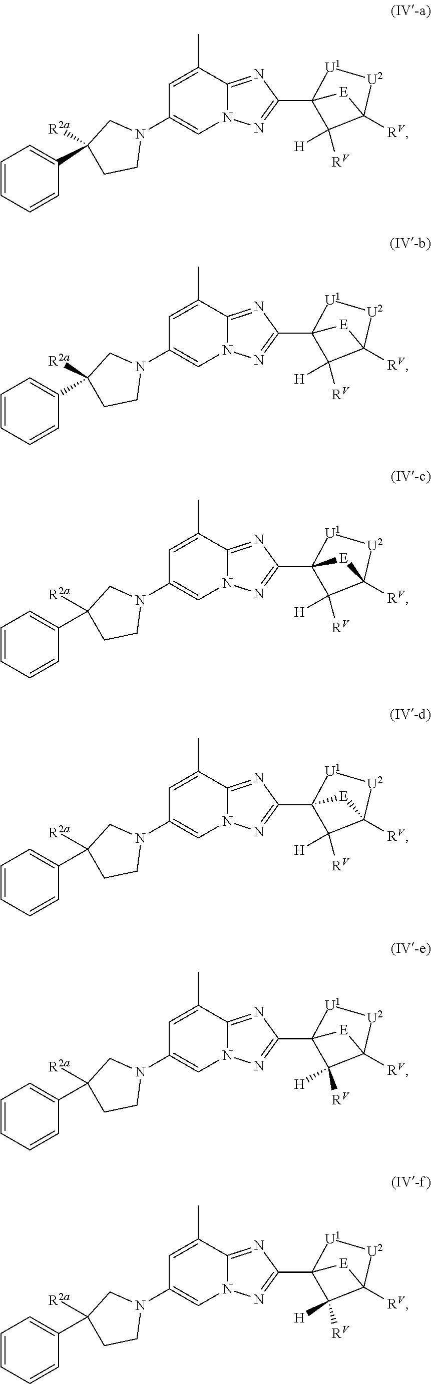

or a pharmaceutically acceptable salt thereof, wherein R^{2a}, E, U¹, U², and R^{V} are each as defined in Formula (I′), or as described herein in any combination.

In some aspects, the present disclosure provides a compound of Formula (V′):

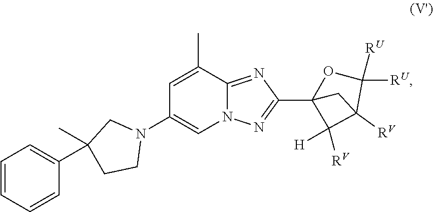

or a pharmaceutically acceptable salt thereof, wherein R^{U} and R^{V} are each as defined in Formula (I′), or as described herein in any combination.

In some aspects, the present disclosure provides a compound of Formula (V′-a), (V′-b), (V′-c), (V′-d), (V′-e), or (V′-f):

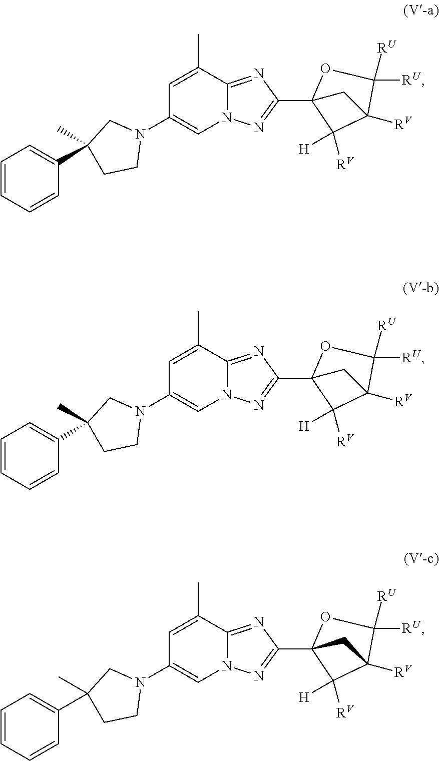

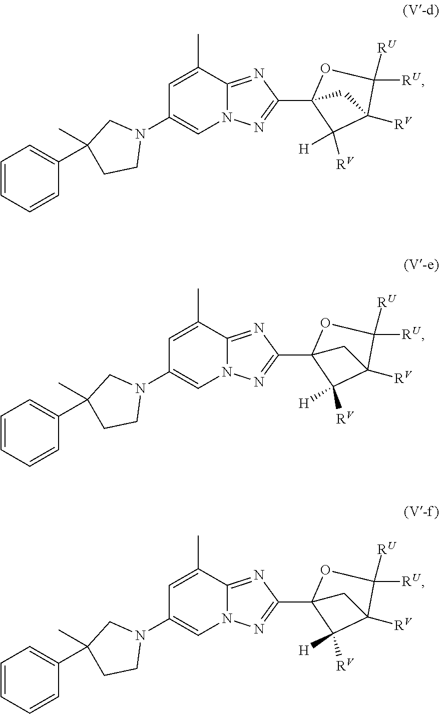

or a pharmaceutically acceptable salt thereof, wherein R^{U} and R^{V} are each as defined in Formula (I′), or as described herein in any combination.

### Variables m, n, and X

In some embodiments, m is 0. In some embodiments, m is 1.

In some embodiments, n is 0. In some embodiments, n is 1.

In some embodiments, X is —CH₂—. In some embodiments, X is —O—.

### Variable R¹

In some embodiments, R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, —NH₂, —NHC(═O)(C₁-C₆ alkyl), —NHS(═O)₂(C₁-C₆ alkyl), or —C(═O)NH₂.

In some embodiments, R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is substituted with one or more halogen, cyano, —OH, —NH₂, —NHC(═O)(C₁-C₆ alkyl), —NHS(═O)₂(C₁-C₆ alkyl), or —C(═O)NH₂.

In some embodiments, R¹ is halogen (e.g., —F, —Cl, —Br, or —I).

In some embodiments, R¹ is cyano.

In some embodiments, R¹ is —OH.

In some embodiments, R¹ is —O(C₁-C₆ alkyl) (e.g., —O(methyl), —O(ethyl), —O(n-propyl), —O(isopropyl), —O(n-butyl), —O(t-butyl), —O(isobutyl), or —O(sec-butyl)).

In some embodiments, R¹ is —NH₂.

In some embodiments, R¹ is —NH(C₁-C₆ alkyl) (e.g., —NH(methyl), —NH(ethyl), —NH(n-propyl), —NH(isopropyl), —NH(n-butyl), —NH(t-butyl), —NH(isobutyl), or —NH(sec-butyl)).

In some embodiments, R¹ is —N(C₁-C₆ alkyl)₂ (e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, R¹ is C₁-C₆ alkyl (e.g., methyl, ethyl, n-propyl, isopropyl, n-butyl, t-butyl, isobutyl, or sec-butyl).

In some embodiments, R¹ is C₁-C₆ alkyl (e.g., methyl, ethyl, n-propyl, isopropyl, n-butyl, t-butyl, isobutyl, or sec-butyl) substituted with one or more halogen, cyano, —OH, —NH₂, —NHC(═O)(C₁-C₆ alkyl), —NHS(═O)₂(C₁-C₆ alkyl), or —C(═O)NH₂.

In some embodiments, R¹ is C₁-C₃ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂.

In some embodiments, R¹ is C₁-C₆ alkyl (e.g., methyl, ethyl, n-propyl, isopropyl, n-butyl, t-butyl, isobutyl, or sec-butyl) substituted with one or more —OH.

In some embodiments, R¹ is C₁-C₆ alkyl (e.g., methyl, ethyl, n-propyl, isopropyl, n-butyl, t-butyl, isobutyl, or sec-butyl) substituted with one or more —NH₂.

In some embodiments, R¹ is C₁-C₆ alkyl (e.g., methyl, ethyl, n-propyl, isopropyl, n-butyl, t-butyl, isobutyl, or sec-butyl) substituted with one or more halogen (e.g., —F, —Cl, —Br, or —I).

In some embodiments, R¹ is C₁-C₆ alkyl (e.g., methyl, ethyl, n-propyl, isopropyl, n-butyl, t-butyl, isobutyl, or sec-butyl) substituted with one or more —NHC(═O)(C₁-C₆ alkyl).

In some embodiments, R¹ is C₁-C₆ alkyl (e.g., methyl, ethyl, n-propyl, isopropyl, n-butyl, t-butyl, isobutyl, or sec-butyl) substituted with one or more —NHS(═O)₂(C₁-C₆ alkyl).

In some embodiments, R¹ is C₁-C₆ alkyl (e.g., methyl, ethyl, n-propyl, isopropyl, n-butyl, t-butyl, isobutyl, or sec-butyl) substituted with one or more —C(═O)NH₂.

In some embodiments, R¹ is methyl.

Variables R^{2a}, R^{2b}, R^{2c}, and R^{2d}

In some embodiments, R^{2a} is C₁-C₆ alkyl (e.g., methyl, ethyl, n-propyl, isopropyl, n-butyl, t-butyl, isobutyl, or sec-butyl) optionally substituted with one or more halogen, cyano, —OH, or —NH₂.

In some embodiments, R^{2a} is C₁-C₆ alkyl (e.g., methyl, ethyl, n-propyl, isopropyl, n-butyl, t-butyl, isobutyl, or sec-butyl) substituted with one or more halogen, cyano, —OH, or —NH₂.

In some embodiments, R^{2a} is C₁-C₆ alkyl (e.g., methyl, ethyl, n-propyl, isopropyl, n-butyl, t-butyl, isobutyl, or sec-butyl).

In some embodiments, R^{2a} is C₁-C₆ alkyl (e.g., methyl, ethyl, n-propyl, isopropyl, n-butyl, t-butyl, isobutyl, or sec-butyl) substituted with one or more halogen (e.g., —F, —Cl, —Br, or —I).

In some embodiments, R^{2a} is C₁-C₆ alkyl (e.g., methyl, ethyl, n-propyl, isopropyl, n-butyl, t-butyl, isobutyl, or sec-butyl) substituted with one or more cyano.

In some embodiments, R^{2a} is C₁-C₆ alkyl (e.g., methyl, ethyl, n-propyl, isopropyl, n-butyl, t-butyl, isobutyl, or sec-butyl) substituted with one or more —OH.

In some embodiments, R^{2a} is C₁-C₆ alkyl (e.g., methyl, ethyl, n-propyl, isopropyl, n-butyl, t-butyl, isobutyl, or sec-butyl) substituted with one or more —NH₂.

In some embodiments, R^{2a} is C₁-C₃ alkyl optionally substituted with one or more halogen, cyano, —OH, or —NH₂. In some embodiments, R^{2a} is C₁ alkyl optionally substituted with one or more halogen, cyano, —OH, or —NH₂. In some embodiments, R^{2a} is C₁-C₃ alkyl. In some embodiments, R^{2a} is methyl.

In some embodiments, R^{2b} is H or C₁-C₆ alkyl (e.g., methyl, ethyl, n-propyl, isopropyl, n-butyl, t-butyl, isobutyl, or sec-butyl), wherein the C₁-C₆ alkyl is optionally substituted with one or more halogen (e.g., —F, —Cl, —Br, or —I).

In some embodiments, R^{2b} is H.

In some embodiments, R^{2b} is C₁-C₆ alkyl (e.g., methyl, ethyl, n-propyl, isopropyl, n-butyl, t-butyl, isobutyl, or sec-butyl).

In some embodiments, R^{2b} is C₁-C₆ alkyl (e.g., methyl, ethyl, n-propyl, isopropyl, n-butyl, t-butyl, isobutyl, or sec-butyl) substituted with one or more halogen (e.g., —F, —Cl, —Br, or —I).

In some embodiments, R^{2c} is H or C₁-C₆ alkyl (e.g., methyl, ethyl, n-propyl, isopropyl, n-butyl, t-butyl, isobutyl, or sec-butyl), wherein the C₁-C₆ alkyl is optionally substituted with one or more halogen (e.g., —F, —Cl, —Br, or —I).

In some embodiments, R^{2c} is H.

In some embodiments, R^{2c} is C₁-C₆ alkyl (e.g., methyl, ethyl, n-propyl, isopropyl, n-butyl, t-butyl, isobutyl, or sec-butyl).

In some embodiments, R^{2c} is C₁-C₆ alkyl (e.g., methyl, ethyl, n-propyl, isopropyl, n-butyl, t-butyl, isobutyl, or sec-butyl) substituted with one or more halogen (e.g., —F, —Cl, —Br, or —I).

In some embodiments, R^{2d} is H.

In some embodiments, R^{2a} and R^{2d} together form C₁-C₆ alkylene (e.g., methylene, ethylene, n-propylene, isopropylene, n-butylene, t-butylene, isobutylene, or sec-butylene) optionally substituted with one or more halogen (e.g., —F, —Cl, —Br, or —I).

In some embodiments, R^{2a} and R^{2d} together form methylene.

In some embodiments, R^{2b}, R^{2c}, and R^{2d} are each H or C₁-C₃ alkyl.

In some embodiments, R^{2b}, R^{2c}, and R^{2d} are each H.

Variables Y, R^{Ya}, and R^{Yb}

In some embodiments, Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya}.

In some embodiments, Y is C₃-C₈ cycloalkyl, C₆-C₁₀ aryl, or 5- to 10-membered heteroaryl.

In some embodiments, Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is substituted with one or more R^{Ya}.

In some embodiments, Y is phenyl or 5- to 6-membered heteroaryl, wherein the phenyl, or 5- to 10-membered heteroaryl is optionally substituted with one or more R^{Ya}.

In some embodiments, Y is C₃-C₈ cycloalkyl (e.g., cyclopropyl, cyclobutyl, cyclopentyl, cyclohexyl, cycloheptyl, cyclooctyl, spiropentyl, spirohexyl, spiroheptyl, or spirooctyl).

In some embodiments, Y is C₃-C₈ monocyclic cycloalkyl.

In some embodiments, Y is C₃-C₈ monocyclic cycloalkyl substituted with one or more R^{Ya}.

In some embodiments, Y is cyclopropyl, cyclobutyl, cyclopentyl, cyclohexyl, cycloheptyl, or cyclooctyl.

In some embodiments, Y is C₅-C₈ spiro bicyclic cycloalkyl.

In some embodiments, Y is C₅-C₈ spiro bicyclic cycloalkyl substituted with one or more R^{Ya}.

In some embodiments, Y is spiropentyl, spirohexyl, spiroheptyl, or spirooctyl.

In some embodiments, Y is spiro[2.2]pentyl, spiro[2.3]hexyl, spiro[2.4]heptyl, spiro[3.3]heptyl, spiro[2.5]octyl, or spiro[3.4]octyl.

In some embodiments, Y is

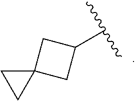 [SMILES: *C1CC2(CC2)C1]

In some embodiments, Y is

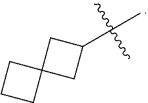 [SMILES: "." ; *C1CC2(CCC2)C1]

In some embodiments, Y is C₆-C₁₀ aryl (e.g., phenyl).

In some embodiments, Y is phenyl.

In some embodiments, Y is 5- to 10-membered heteroaryl.

In some embodiments, Y is 5- to 10-membered heteroaryl containing at least one heteroatom selected from N, O, and S.

In some embodiments, Y is 5-membered heteroaryl containing at least one heteroatom selected from N, O, and S.

In some embodiments, Y is 5-membered heteroaryl containing one heteroatom selected from N, O, and S (e.g., pyrrolyl, furanyl, or thiophenyl).

In some embodiments, Y is 5-membered heteroaryl containing two heteroatoms selected from N, O, and S (e.g., oxazolyl, thiazolyl, pyrazolyl, isoxazolyl, isothiazolyl, or imidazolyl).

In some embodiments, Y is oxazolyl.

In some embodiments, Y is

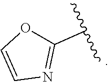 [SMILES: *c1ncco1]

In some embodiments, Y is thiazolyl.

In some embodiments, Y is

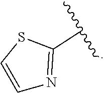 [SMILES: *c1nccs1]

In some embodiments, Y is pyrazolyl.

In some embodiments, Y is

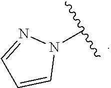 [SMILES: *n1cccn1]

In some embodiments, Y is

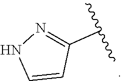 [SMILES: *c1cc[nH]n1]

In some embodiments, Y is isoxazolyl.

In some embodiments, Y is isothiazolyl.

In some embodiments, Y is imidazolyl.

In some embodiments, Y is 5-membered heteroaryl containing three heteroatoms selected from N, O, and S (e.g., triazolyl, oxadiazolyl, thiadiazolyl, or oxathiadiazolyl).

In some embodiments, Y is triazolyl.

In some embodiments, Y is

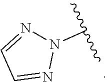 [SMILES: *n1nccn1]

In some embodiments, Y is oxadiazolyl.

In some embodiments, Y is

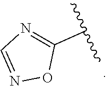 [SMILES: *c1ncno1]

In some embodiments, Y is thiadiazolyl.

In some embodiments, Y is

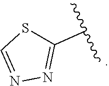 [SMILES: *c1nncs1]

In some embodiments, Y is

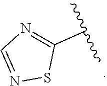 [SMILES: *c1ncns1]

In some embodiments, Y is 5-membered heteroaryl containing four heteroatoms selected from N, O, and S (e.g., tetrazolyl or oxatriazolyl)

In some embodiments, Y is tetrazolyl.

In some embodiments, Y is

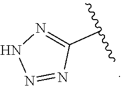 [SMILES: *c1nn[nH]n1]

In some embodiments, Y is oxatriazolyl.

In some embodiments, Y is 6-membered heteroaryl containing at least one heteroatom selected from N, O, and S.

In some embodiments, Y is 6-membered heteroaryl containing one heteroatom selected from N, O, and S (e.g., pyridinyl, pyranyl, or thiopyranyl).

In some embodiments, Y is pyridinyl.

In some embodiments, Y is

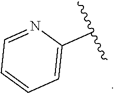 [SMILES: *c1ccccn1]

In some embodiments, Y is 6-membered heteroaryl containing two heteroatoms selected from N, O, and S (e.g., pyrimidinyl, pyridazinyl, or pyrazinyl).

In some embodiments, Y is pyrimidinyl.

In some embodiments, Y is

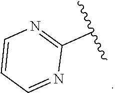 [SMILES: *c1ncccn1]

In some embodiments, Y is pyridazinyl.

In some embodiments, Y is pyrazinyl.

In some embodiments, at least one R^{Ya} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb}.

In some embodiments, at least one R^{Ya} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

In some embodiments, at least one R^{Ya} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is substituted with one or more R^{Yb}.

In some embodiments, at least one R^{Ya} is oxo.

In some embodiments, at least one R^{Ya} is halogen (e.g., e.g., —F, —Cl, —Br, or —I).

In some embodiments, at least one R^{Ya} is —F.

In some embodiments, at least one R^{Ya} is —C₁.

In some embodiments, at least one R^{Ya} is —Br.

In some embodiments, at least one R^{Ya} is —I.

In some embodiments, at least one R^{Ya} is cyano.

In some embodiments, at least one R^{Ya} is —OH.

In some embodiments, at least one R^{Ya} is —O(C₁-C₆ alkyl) (e.g., —OCH₃, —OCH₂CH₃, —O(CH₂)₂CH₃, —O(CH₂)₃CH₃, —O(CH₂)₄CH₃, or —O(CH₂)₅CH₃).

In some embodiments, at least one R^{Ya} is —OCH₃.

In some embodiments, at least one R^{Ya} is —OCH₂CH₃.

In some embodiments, at least one R^{Ya} is —NH₂.

In some embodiments, at least one R^{Ya} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{Ya} is —N(C₁-C₆ alkyl)₂.

In some embodiments, at least one R^{Ya} is —C(═O)—H.

In some embodiments, at least one R^{Ya} is —C(═O)—OH.

In some embodiments, at least one R^{Ya} is —C(═O)—O(C₁-C₆ alkyl) (e.g., is —C(═O)—OCH₃, —C(═O)—OCH₂CH₃, —C(═O)—O(CH₂)₂CH₃, —C(═O)—O(CH₂)₃CH₃, —C(═O)—O(CH₂)₄CH₃, or —C(═O)—O(CH₂)₅CH₃).

In some embodiments, at least one R^{Ya} is —C(═O)—NH₂.

In some embodiments, at least one R^{Ya} is —C(═O)—NH(C₁-C₆ alkyl) (e.g., —C(═O)—NHCH₃, —C(═O)—NHCH₂CH₃, —C(═O)—NH(CH₂)₂CH₃, —C(═O)—NH(CH₂)₃CH₃, —C(═O)—NH(CH₂)₄CH₃, or —C(═O)—NH(CH₂)₅CH₃).

In some embodiments, at least one R^{Ya} is —C(═O)—N(C₁-C₆ alkyl)₂. (e.g., —C(═O)—N(methyl)₂, —C(═O)—N(ethyl)₂, —C(═O)—N(n-propyl)₂, —C(═O)—N(isopropyl)₂, —C(═O)—N(n-butyl)₂, —C(═O)—N(t-butyl)₂, —C(═O)—N(isobutyl)₂, or —C(═O)—N(sec-butyl)₂).

In some embodiments, at least one R^{Ya} is C₂-C₆ alkenyl.

In some embodiments, at least one R^{Ya} is C₂-C₆ alkynyl.

In some embodiments, at least one R^{Ya} is —C₁-C₆ alkyl (e.g., methyl, ethyl, n-propyl, isopropyl, n-butyl, t-butyl, isobutyl, or sec-butyl).

In some embodiments, at least one R^{Ya} is methyl.

In some embodiments, at least one R^{Ya} is ethyl.

In some embodiments, at least one R^{Yb} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂.

In some embodiments, at least one R^{Yb} is oxo.

In some embodiments, at least one R^{Yb} is halogen (e.g., e.g., —F, —Cl, —Br, or —I).

In some embodiments, at least one R^{Yb} is —F.

In some embodiments, at least one R^{Yb} is —C₁.

In some embodiments, at least one R^{Yb} is —Br.

In some embodiments, at least one R^{Yb} is —I.

In some embodiments, at least one R^{Yb} is cyano.

In some embodiments, at least one R^{Yb} is —OH.

In some embodiments, at least one R^{Yb} is —O(C₁-C₆ alkyl) (e.g., —OCH₃, —OCH₂CH₃, —O(CH₂)₂CH₃, —O(CH₂)₃CH₃, —O(CH₂)₄CH₃, or —O(CH₂)₅CH₃).

In some embodiments, at least one R^{Yb} is —OCH₃.

In some embodiments, at least one R^{Yb} is —OCH₂CH₃.

In some embodiments, at least one R^{Yb} is —NH₂.

In some embodiments, at least one R^{Yb} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{Yb} is —N(C₁-C₆ alkyl)₂ (e.g., e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, at least one R^{Yb} is —C(═O)—H.

In some embodiments, at least one R^{Yb} is —C(═O)—OH.

In some embodiments, at least one R^{Yb} is —C(═O)—O(C₁-C₆ alkyl) (e.g., is —C(═O)—OCH₃, —C(═O)—OCH₂CH₃, —C(═O)—O(CH₂)₂CH₃, —C(═O)—O(CH₂)₃CH₃, —C(═O)—O(CH₂)₄CH₃, or —C(═O)—O(CH₂)₅CH₃).

In some embodiments, at least one R^{Yb} is —C(═O)—NH₂.

In some embodiments, at least one R^{Yb} is —C(═O)—NH(C₁-C₆ alkyl) ((e.g., —C(═O)—NHCH₃, —C(═O)—NHCH₂CH₃, —C(═O)—NH(CH₂)₂CH₃, —C(═O)—NH(CH₂)₃CH₃, —C(═O)—NH(CH₂)₄CH₃, or —C(═O)—NH(CH₂)₅CH₃)).

In some embodiments, at least one R^{Yb} is —C(═O)—N(C₁-C₆ alkyl)₂ (e.g., —C(═O)—N(methyl)₂, —C(═O)—N(ethyl)₂, —C(═O)—N(n-propyl)₂, —C(═O)—N(isopropyl)₂, —C(═O)—N(n-butyl)₂, —C(═O)—N(t-butyl)₂, —C(═O)—N(isobutyl)₂, or —C(═O)—N(sec-butyl)₂).

Variables T, R^{T1}, R^{T2}, R^{Ta}, R^{Tb}, R^{Tb1}, R^{Tc}, R^{b}, R^{c}, Ring A, and Ring B

### Variable T

In some embodiments, T is —OR^{T1}, —N(R^{T1})₂, or —N(R^{T2})₂.

In some embodiments, T is —OR^{T1}.

In some embodiments, T is —N(R^{T1})₂.

In some embodiments, T is —NH^{T1} or —N(C₁-C₆ alkyl)R^{T1}. In some embodiments, T is —NH^{T1} or —N(C₁-C₃ alkyl)R^{T}1. In some embodiments, T is —NHR^{T1} or —N(CH₃)R^{T1}.

In some embodiments, T is —N(R^{T2})₂.

### Variable R^{T1}

In some embodiments, at least one R^{T1} is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta}.

In some embodiments, at least one R^{T1} is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl).

In some embodiments, at least one R^{T1} is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is substituted with one or more R^{Ta}.

In some embodiments, one R^{T1} is H or C₁-C₆ alkyl; and the other R^{T1} is C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta}.

In some embodiments, one R^{T1} is H or C₁-C₆ alkyl; and the other R^{T1} is C₁-C₆ alkyl, C₂-C₆ alkenyl, C₂-C₆ alkynyl, monocyclic C₃-C₈ cycloalkyl, fused bicyclic C₄-C₈ cycloalkyl, bridged bicyclic C₅-C₈ cycloalkyl, spiro bicyclic C₅-C₈ cycloalkyl, monocyclic 4- to 8-membered heterocyclyl, fused bicyclic 4- to 10-membered heterocyclyl, bridged bicyclic 5- to 10-membered heterocyclyl, spiro bicyclic 5- to 10-membered heterocyclyl, phenyl, monocyclic 5- to 6-membered heteroaryl, fused bicyclic 5- to 10-membered heteroaryl, —(C₁-C₄ alkyl)-(monocyclic C₃-C₈ cycloalkyl), —(C₁-C₄ alkyl)-(fused bicyclic C₄-C₈ cycloalkyl), —(C₁-C₄ alkyl)-(bridged bicyclic C₅-C₈ cycloalkyl), —(C₁-C₄ alkyl)-(spiro bicyclic C₅-C₈ cycloalkyl), —(C₁-C₄ alkyl)-(monocyclic 4- to 7-membered heterocyclyl), —(C₁-C₄ alkyl)-(fused bicyclic 4- to 10-membered heterocyclyl), —(C₁-C₄ alkyl)-(bridged bicyclic 5- to 10-membered heterocyclyl), —(C₁-C₄ alkyl)-(spiro bicyclic 5- to 10-membered heterocyclyl), —(C₁-C₄ alkyl)-(phenyl), —(C₁-C₄ alkyl)-(monocyclic 5- to 6-membered heteroaryl), or —(C₁-C₄ alkyl)-(fused bicyclic 5- to 10-membered heteroaryl), wherein the other R^{T1} is optionally substituted with one or more R^{Ta}.

In some embodiments, at least one R^{T1} is H.

In some embodiments, at least one R^{T1} is C₁-C₆ alkyl (e.g., methyl, ethyl, n-propyl, isopropyl, n-butyl, t-butyl, isobutyl, or sec-butyl).

In some embodiments, at least one R^{T1} is C₁-C₆ alkyl (e.g., methyl, ethyl, n-propyl, isopropyl, n-butyl, t-butyl, isobutyl, or sec-butyl) substituted with one or more R^{Ta}.

In some embodiments, at least one R^{T1} is methyl.

In some embodiments, at least one R^{T1} is ethyl.

In some embodiments, at least one R^{T1} is n-propyl.

In some embodiments, at least one R^{T1} is isopropyl.

In some embodiments, at least one R^{T1} is n-butyl.

In some embodiments, at least one R^{T1} is C₂-C₆ alkenyl.

In some embodiments, at least one R^{T1} is C₂-C₆ alkenyl substituted with one or more R^{Ta}.

In some embodiments, at least one R^{T1} is C₂-C₆ alkynyl.

In some embodiments, at least one R^{T1} is C₂-C₆ alkynyl substituted with one or more R^{Ta}.

In some embodiments, at least one R^{T1} is C₃-C₈ cycloalkyl.

In some embodiments, at least one R^{T1} is C₃-C₈ cycloalkyl substituted with one or more R^{Ta}.

In some embodiments, at least one R^{T1} is C₃-C₈ monocyclic cycloalkyl.

In some embodiments, at least one R^{T1} is C₃-C₈ monocyclic cycloalkyl substituted with one or more R^{Ta}.

In some embodiments, at least one R^{T1} is cyclopropyl, cyclobutyl, cyclopentyl, cyclohexyl, cycloheptyl, or cyclooctyl.

In some embodiments, at least one R^{T1} is C₄-C₈ bicyclic cycloalkyl.

In some embodiments, at least one R^{T1} is C₄-C₈ bicyclic cycloalkyl substituted with one or more R^{Ta}.

In some embodiments, at least one R^{T1} is bicyclocyclobutyl, bicyclocyclopentyl, bicyclocyclohexyl, bicyclocycloheptyl, or bicyclocyclooctyl.

In some embodiments, at least one R^{T1} is C₄-C₈ fused bicyclic cycloalkyl.

In some embodiments, at least one R^{T1} is C₄-C₈ fused bicyclic cycloalkyl substituted with one or more R^{Ta}.

In some embodiments, at least one R^{T1} is

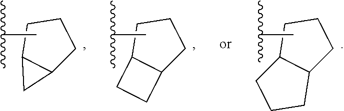

In some embodiments, at least one R^{T1} is

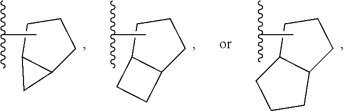

substituted with one or more R^{Ta}.

In some embodiments, at least one R^{T1} is

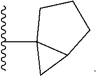 [SMILES: *C12CCCC1C2]

In some embodiments, at least one R^{T1} is

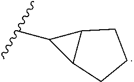 [SMILES: *C1C2CCCC12]

In some embodiments, at least one R^{T1} is C₃-C₈ bridged bicyclic cycloalkyl.

In some embodiments, at least one R^{T1} is C₃-C₈ bridged bicyclic cycloalkyl substituted with one or more R^{Ta}.

In some embodiments, at least one R^{T1} is

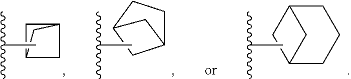

In some embodiments, at least one R^{T1} is

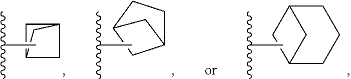

substituted with one or more R^{Ta}.

In some embodiments, at least one R^{T1} is

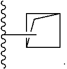

In some embodiments, at least one R^{T1} is

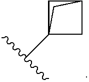 [SMILES: *C12CC(C1)C2]

In some embodiments, at least one R^{T1} is

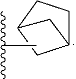

In some embodiments, at least one R^{T1} is

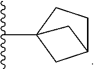 [SMILES: *C12CCC(C1)C2]

In some embodiments, at least one R^{T1} is

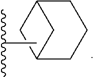

In some embodiments, at least one R^{T1} is

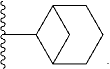 [SMILES: *C1C2CCCC1C2]

In some embodiments, at least one R^{T1} is C₅-C₈ spiro bicyclic cycloalkyl.

In some embodiments, at least one R^{T1} is C₅-C₈ spiro bicyclic cycloalkyl substituted with one or more R^{Ta}.

In some embodiments, at least one R^{T1} is spiro[2.2]pentyl, spiro[2.3]hexyl, spiro[2.4]heptyl, spiro[3.3]heptyl, spiro[2.5]octyl, or spiro[3.4]octyl.

In some embodiments, at least one R^{T1} is spiro[2.2]pentyl, spiro[2.3]hexyl, spiro[2.4]heptyl, spiro[3.3]heptyl, spiro[2.5]octyl, or spiro[3.4]octyl, substituted with one or more R^{Ta}.

In some embodiments, at least one R^{T1} is

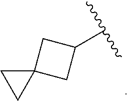 [SMILES: *C1CC2(CC2)C1]

In some embodiments, at least one R^{T1} is

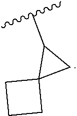 [SMILES: *C1CC12CCC2]

In some embodiments, at least one R^{T1} is

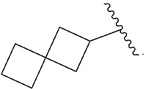 [SMILES: *C1CC2(CCC2)C1 ; "."]

In some embodiments, at least one R^{T1} is 4- to 10-membered heterocyclyl.

In some embodiments, at least one R^{T1} is 4- to 10-membered heterocyclyl substituted with one or more R^{Ta}.

In some embodiments, at least one R^{T1} is 4- to 10-membered monocyclic heterocyclyl.

In some embodiments, at least one R^{T1} is 4- to 10-membered monocyclic heterocyclyl substituted with one or more R^{Ta}.

In some embodiments, at least one R^{T1} is 4- to 10-membered bicyclic heterocyclyl.

In some embodiments, at least one R^{T1} is 4- to 10-membered bicyclic heterocyclyl substituted with one or more R^{Ta}.

In some embodiments, at least one R^{T1} is 4- to 10-membered fused bicyclic heterocyclyl.

In some embodiments, at least one R^{T1} is 4- to 10-membered fused bicyclic heterocyclyl substituted with one or more R^{Ta}.

In some embodiments, at least one R^{T1} is 5- to 10-membered bridged bicyclic heterocyclyl.

In some embodiments, at least one R^{T1} is 5- to 10-membered bridged bicyclic heterocyclyl substituted with one or more R^{Ta}.

In some embodiments, at least one R^{T1} is 5- to 10-membered spiro bicyclic heterocyclyl.

In some embodiments, at least one R^{T1} is 5- to 10-membered spiro bicyclic heterocyclyl substituted with one or more R^{Ta}.

In some embodiments, at least one R^{T1} is 4- to 10-membered heterocyclyl containing at least one heteroatom selected from N, O, and S.

In some embodiments, at least one R^{T1} is 4- to 10-membered heterocyclyl containing at least one heteroatom selected from N, O, and S, wherein the 4- to 10-membered heterocyclyl is substituted with one or more R^{Ta}.

In some embodiments, at least one R^{T1} is 4- to 10-membered monocyclic heterocyclyl containing at least one heteroatom selected from N, O, and S.

In some embodiments, at least one R^{T1} is 4- to 10-membered monocyclic heterocyclyl containing at least one heteroatom selected from N, O, and S, wherein the 4- to 10-membered heterocyclyl is substituted with one or more R^{Ta}.

In some embodiments, at least one R^{T1} is 4- to 10-membered monocyclic heterocyclyl containing one N (e.g., azetidinyl, pyrrolidinyl, or piperidinyl).

In some embodiments, at least one R^{T1} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Ta}.

In some embodiments, at least one R^{T1} is azetidinyl.

In some embodiments, at least one R^{T1} is azetidinyl substituted with one or more R^{Ta}.

In some embodiments, at least one R^{T1} is

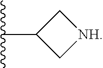 [SMILES: *C1CNC1]

In some embodiments, at least one R^{T1} is

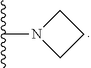 [SMILES: *N1CCC1]

In some embodiments, at least one R^{T1}

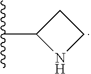 [SMILES: *C1CCN1]

In some embodiments, at least one R^{T1} is pyrrolidinyl.

In some embodiments, at least one R^{T1} is

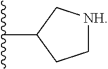 [SMILES: *C1CCNC1]

In some embodiments, at least one R^{T1} is

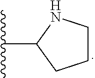 [SMILES: *C1CCCN1]

In some embodiments, at least one R^{T1} is

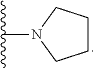 [SMILES: *N1CCCC1]

In some embodiments, at least one R^{T1} is piperidinyl.

In some embodiments, at least one R^{T1} is

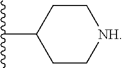 [SMILES: *C1CCNCC1]

In some embodiments, at least one R^{T1} is

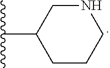 [SMILES: *C1CCCNC1]

In some embodiments, at least one R^{T1} is

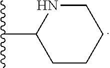 [SMILES: *C1CCCCN1]

In some embodiments, at least one R^{T1} is

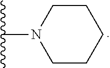 [SMILES: *N1CCCCC1]

In some embodiments, at least one R^{T1} is 4- to 10-membered fused bicyclic heterocyclyl containing one N.

In some embodiments, at least one R^{T1} is 4- to 10-membered fused bicyclic heterocyclyl containing one N, wherein the 4- to 10-membered fused bicyclic heterocyclyl is substituted with one or more R^{Ta}.

In some embodiments, at least one R^{T1} is

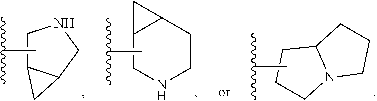

In some embodiments, at least one R^{T1} is

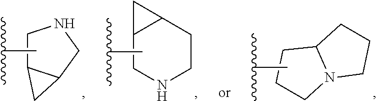

substituted with one or more R^{Ta}.

In some embodiments, at least one R^{T1} is

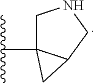 [SMILES: *C12CNCC1C2]

In some embodiments, at least one R^{T1} is

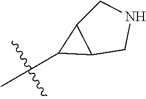 [SMILES: *C1C2CNCC12]

In some embodiments, at least one R^{T1} is H

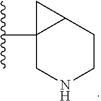 [SMILES: *C12CNCCC1C2]

In some embodiments, at least one R^{T1} is

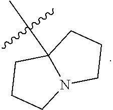 [SMILES: *C12CCCN1CCC2]

In some embodiments, at least one R^{T1} is 5- to 10-membered bridged bicyclic heterocyclyl containing one N.

In some embodiments, at least one R^{T1} is 5- to 10-membered bridged bicyclic heterocyclyl containing one N, wherein the 5- to 10-membered bridged bicyclic heterocyclyl is substituted with one or more R^{Ta}.

In some embodiments, at least one R^{T1} is

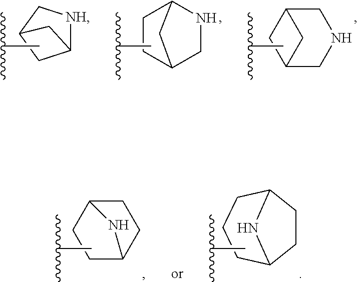

In some embodiments, at least one R^{T1} is

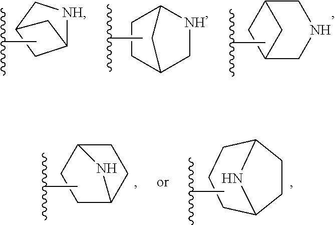

or substituted with one or more R^{Ta}.

In some embodiments, at least one R^{T1} is

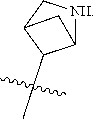 [SMILES: *C1C2CNC1C2]

In some embodiments, at least one R^{T1} is

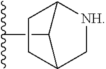 [SMILES: *C1C2CCC1NC2]

In some embodiments, at least one R^{T1} is

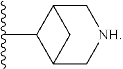 [SMILES: *C1C2CNCC1C2]

In some embodiments, at least one R^{T1} is

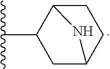 [SMILES: *C1CC2CCC1N2]

In some embodiments, at least one R^{T1} is

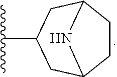 [SMILES: *C1CC2CCC(C1)N2]

In some embodiments, at least one R^{T1} is

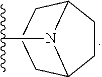 [SMILES: *N1C2CCCC1CC2]

In some embodiments, at least one R^{T1} is 5- to 10-membered spiro bicyclic heterocyclyl containing one N.

In some embodiments, at least one R^{T1} is 5- to 10-membered spiro bicyclic heterocyclyl containing one N, wherein the 5- to 10-membered spiro bicyclic heterocyclyl is substituted with one or more R^{Ta}.

In some embodiments, at least one R^{T1} is

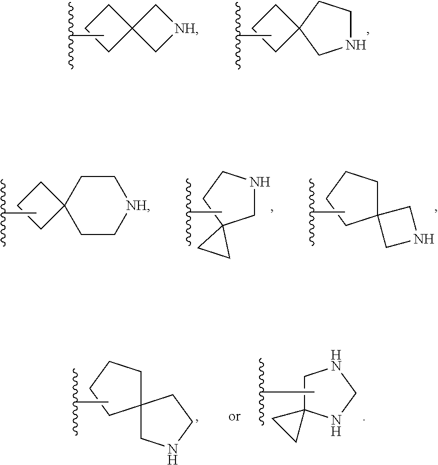

In some embodiments, at least one R^{T1} is

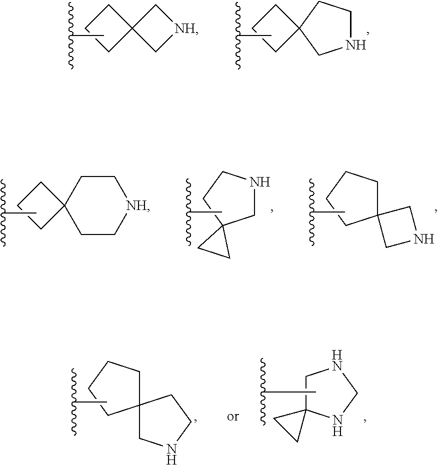

substituted with one or more R^{Ta}.

In some embodiments, at least one R^{T1} is

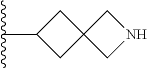 [SMILES: *C1CC2(CNC2)C1]

In some embodiments, at least one R^{T1} is

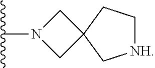 [SMILES: *N1CC2(CCNC2)C1]

In some embodiments, at least one R^{T1} is

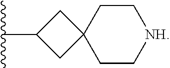 [SMILES: *C1CC2(CCNCC2)C1]

In some embodiments, at least one R^{T1} is

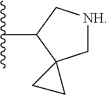 [SMILES: *C1CNCC12CC2]

In some embodiments, at least one R^{T1} is

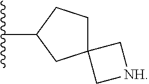 [SMILES: *C1CCC2(CNC2)C1]

In some embodiments, at least one R^{T1} is

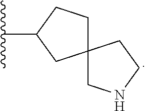 [SMILES: *C1CCC2(CCNC2)C1]

In some embodiments, at least one R^{T1} is 4- to 10-membered monocyclic heterocyclyl containing one O (e.g., oxetanyl, tetrahydrofuranyl, or tetrahydropyranyl).

In some embodiments, at least one R^{T1} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Ta}.

In some embodiments, at least one R^{T1} is oxetanyl.

In some embodiments, at least one R^{T1} is

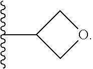 [SMILES: *C1COC1]

In some embodiments, at least one R^{T1} is

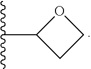 [SMILES: *C1CCO1]

In some embodiments, at least one R^{T1} is tetrahydrofuranyl.

In some embodiments, at least one R^{T1} is

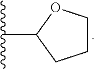 [SMILES: *C1CCCO1]

In some embodiments, at least one R^{T1} is

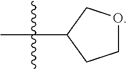 [SMILES: *C1CCOC1]

In some embodiments, at least one R^{T1} is tetrahydropyranyl.

In some embodiments, at least one R^{T1} is

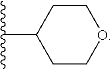 [SMILES: *C1CCOCC1]

In some embodiments, at least one R^{T1} is

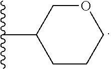 [SMILES: *C1CCCOC1]

In some embodiments, at least one R^{T1} is

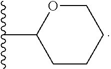 [SMILES: *C1CCCCO1]

In some embodiments, at least one R^{T1} is 5- to 10-membered bridged bicyclic heterocyclyl containing one O.

In some embodiments, at least one R^{T1} is 5- to 10-membered bridged bicyclic heterocyclyl containing one O, wherein the 5- to 10-membered bridged bicyclic heterocyclyl is substituted with one or more R^{Ta}.

In some embodiments, at least one R^{T1} is 4- to 10-membered fused bicyclic heterocyclyl containing one O.

In some embodiments, at least one R^{T1} is 4- to 10-membered fused bicyclic heterocyclyl containing one O, wherein the 4- to 10-membered fused heterocyclyl is substituted with one or more R^{Ta}.

In some embodiments, at least one R^{T1} is 5- to 10-membered spiro bicyclic heterocyclyl containing one O.

In some embodiments, at least one R^{T1} is 5- to 10-membered spiro bicyclic heterocyclyl containing one O, wherein the is 5- to 10-membered spiro bicyclic heterocyclyl is substituted with one or more R^{Ta}.

In some embodiments, at least one R^{T1} is

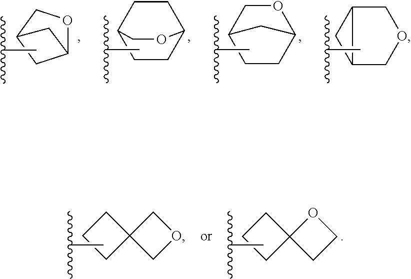

In some embodiments, at least one R^{T1} is

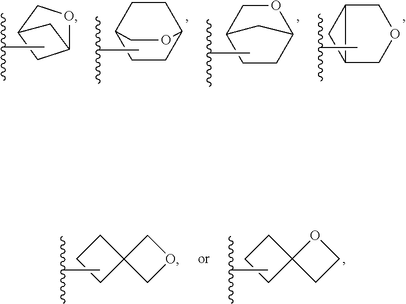

substituted with one or more R^{Ta}.

In some embodiments, at least one R^{T1} is

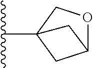 [SMILES: *C12COC(C1)C2]

In some embodiments, at least one R^{T1} is

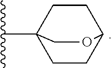 [SMILES: *C12CCC(CC1)OC2]

In some embodiments, at least one R^{T1} is

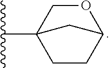 [SMILES: *C12CCC(C1)OC2]

In some embodiments, at least one R^{T1} is

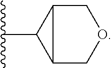 [SMILES: *C1C2COCC12]

In some embodiments, at least one R^{T1} is

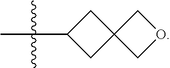 [SMILES: *C1CC2(COC2)C1]

In some embodiments, at least one R^{T1} is

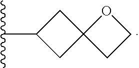 [SMILES: *C1CC2(CCO2)C1]

In some embodiments, at least one R^{T1} is 4- to 10-membered monocyclic heterocyclyl containing one S (e.g., tetrahydrothiophenyl).

In some embodiments, at least one R^{T1} is 4- to 10-membered monocyclic heterocyclyl containing one S (e.g., tetrahydrothiophenyl), wherein the 4- to 10-membered monocyclic heterocyclyl is substituted with one or more R^{Ta}.

In some embodiments, at least one R^{T1} is tetrahydrothiophenyl.

In some embodiments, at least one R^{T1} is

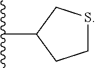 [SMILES: *C1CCSC1]

In some embodiments, at least one R^{T1} is

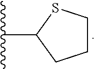 [SMILES: *C1CCCS1]

In some embodiments, at least one R^{T1} is 4- to 10-membered heterocyclyl (e.g., monocyclic, fused bicyclic, bridged bicyclic, or spiro bicyclic) containing two heteroatoms selected from N, O, and S.

In some embodiments, at least one R^{T1} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Ta}.

In some embodiments, at least one R^{T1} is 4- to 10-membered monocyclic heterocyclyl containing two heteroatoms selected from N, O, and S.

In some embodiments, at least one R^{T1} is 4- to 10-membered monocyclic heterocyclyl containing two heteroatoms selected from N, O, and S, wherein the 4- to 10-membered monocyclic heterocyclyl is substituted with one or more R^{Ta}.

In some embodiments, at least one R^{T1} is 4- to 10-membered fused bicyclic heterocyclyl containing two heteroatoms selected from N, O, and S.

In some embodiments, at least one R^{T1} is 4- to 10-membered fused bicyclic heterocyclyl containing two heteroatoms selected from N, O, and S, wherein the 4- to 10-membered fused bicyclic heterocyclyl is substituted with one or more R^{Ta}.

In some embodiments, at least one R^{T1} is 5- to 10-membered bridged bicyclic heterocyclyl containing two heteroatoms selected from N, O, and S.

In some embodiments, at least one R^{T1} is 5- to 10-membered bridged bicyclic heterocyclyl containing two heteroatoms selected from N, O, and S, wherein the 5- to 10-membered bridged bicyclic heterocyclyl is substituted with one or more R^{Ta}.

In some embodiments, at least one R^{T1} is 5- to 10-membered spiro bicyclic heterocyclyl containing two heteroatoms selected from N, O, and S.

In some embodiments, at least one R^{T1} is 5- to 10-membered spiro bicyclic heterocyclyl containing two heteroatoms selected from N, O, and S, wherein the 5- to 10-membered spiro bicyclic heterocyclyl is substituted with one or more R^{Ta}.

In some embodiments, at least one R^{T1} is

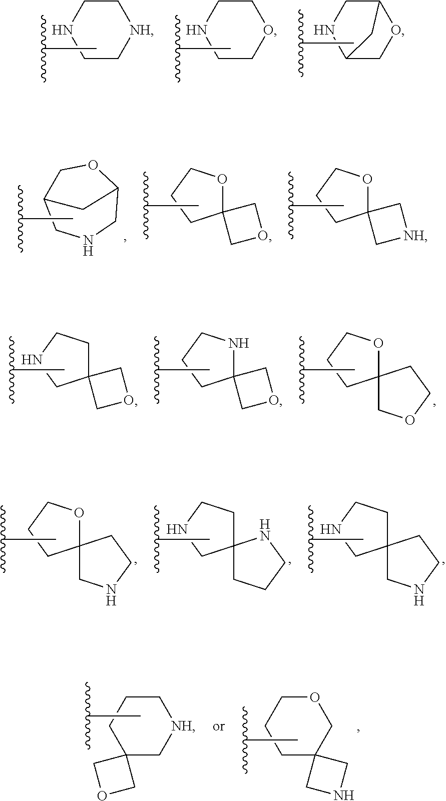

In some embodiments, at least one R^{T1} is

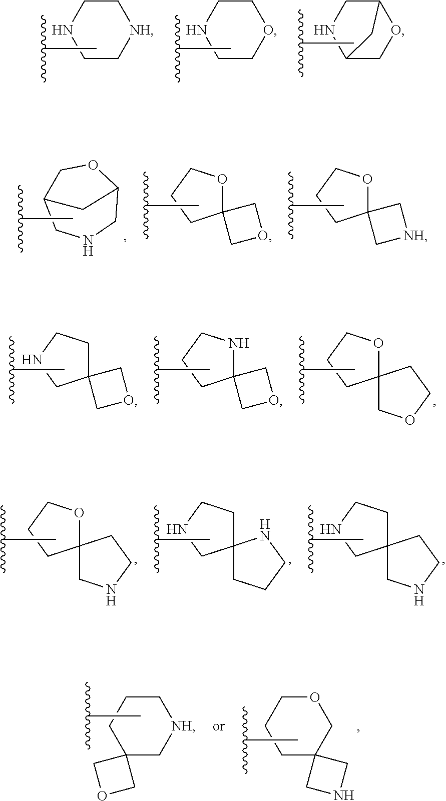

substituted with one or more R^{Ta}.

In some embodiments, at least one R^{T1} is

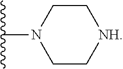 [SMILES: *N1CCNCC1]

In some embodiments, at least one R^{T1} is

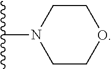 [SMILES: *N1CCOCC1]

In some embodiments, at least one R^{T1} is

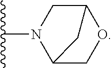 [SMILES: *N1CC2CC1CO2]

In some embodiments, at least one R^{T1} is H

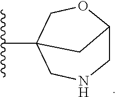 [SMILES: *C12CNCC(C1)OC2]

In some embodiments, at least one R^{T1} is

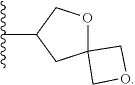 [SMILES: *C1COC2(COC2)C1]

In some embodiments, at least one R^{T1} is

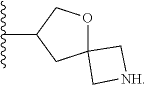 [SMILES: *C1COC2(CNC2)C1]

In some embodiments, at least one R^{T1} is

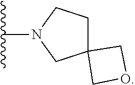 [SMILES: *N1CCC2(COC2)C1]

In some embodiments, at least one R^{T1} is

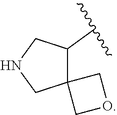 [SMILES: *C1CNCC12COC2]

In some embodiments, at least one R^{T1} is

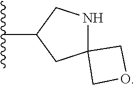 [SMILES: *C1CNC2(COC2)C1]

In some embodiments, at least one R^{T1} is

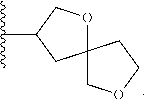 [SMILES: *C1COC2(CCOC2)C1]

In some embodiments, at least one R^{T1} is

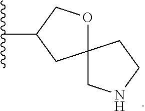 [SMILES: *C1COC2(CCNC2)C1]

In some embodiments, at least one R^{T1} is

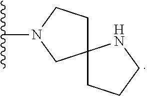 [SMILES: *N1CCC2(CCCN2)C1]

In some embodiments, at least one R^{T1} is

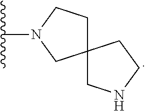 [SMILES: *N1CCC2(CCNC2)C1]

In some embodiments, at least one R^{T1} is

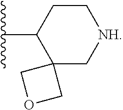 [SMILES: *C1CCNCC12COC2]

In some embodiments, at least one R^{T1} is

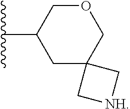 [SMILES: *C1COCC2(CNC2)C1]

In some embodiments, at least one R^{T1} is C₆-C₁₀ aryl (e.g., phenyl).

In some embodiments, at least one R^{T1} is C₆-C₁₀ aryl (e.g., phenyl) substituted with one or more R^{Ta}.

In some embodiments, at least one R^{T1} is phenyl.

In some embodiments, at least one R^{T1} is 5- to 10-membered heteroaryl.

In some embodiments, at least one R^{T1} is 5- to 10-membered heteroaryl substituted with one or more R^{Ta}.

In some embodiments, at least one R^{T1} is 5- to 10-membered heteroaryl containing at least one heteroatom selected from N, O, and S.

In some embodiments, at least one R^{T1} is 5- to 10-membered heteroaryl containing at least one heteroatom selected from N, O, and S, wherein the heteroaryl is substituted with one or more R^{Ta}.

In some embodiments, at least one R^{T1} is 5-membered heteroaryl containing at least one heteroatom selected from N, O, and S.

In some embodiments, at least one R^{T1} is 5-membered heteroaryl containing at least one heteroatom selected from N, O, and S, wherein the heteroaryl is substituted with one or more R^{Ta}.

In some embodiments, at least one R^{T1} is 5-membered heteroaryl containing one heteroatom selected from N, O, and S (e.g., pyrrolyl, furanyl, or thiophenyl).

In some embodiments, at least one R^{T1} is 5-membered heteroaryl containing one heteroatom selected from N, O, and S (e.g., pyrrolyl, furanyl, or thiophenyl), wherein the heteroaryl is substituted with one or more R^{Ta}.

In some embodiments, at least one R^{T1} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{T1} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Ta}.

In some embodiments, at least one R^{T1} is oxazolyl.

In some embodiments, at least one R^{T1} is

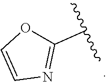 [SMILES: *c1ncco1]

In some embodiments, at least one R^{T1} is thiazolyl.

In some embodiments, at least one R^{T1} is

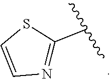 [SMILES: *c1nccs1]

In some embodiments, at least one R^{T1} is pyrazolyl.

In some embodiments, at least one R^{T1} is

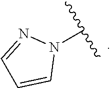 [SMILES: *n1cccn1]

In some embodiments, at least one R^{T1} is

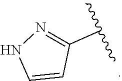 [SMILES: *c1cc[nH]n1]

In some embodiments, at least one R^{T1} is isoxazolyl.

In some embodiments, at least one R^{T1} is isothiazolyl.

In some embodiments, at least one R^{T1} is imidazolyl.

In some embodiments, at least one R^{T1} is 5-membered heteroaryl containing three heteroatoms selected from N, O, and S (e.g., triazolyl, oxadiazolyl, thiadiazolyl, or oxathiadiazolyl).

In some embodiments, at least one R^{T1} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Ta}.

In some embodiments, at least one R^{T1} is triazolyl.

In some embodiments, at least one R^{T1} is

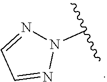 [SMILES: *n1nccn1]

In some embodiments, at least one R^{T1} is

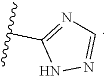 [SMILES: *c1ncn[nH]1]

In some embodiments, at least one R^{T1} is

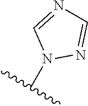 [SMILES: *n1cncn1]

In some embodiments, at least one R^{T1} is oxadiazolyl.

In some embodiments, at least one R^{T1} is

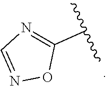 [SMILES: *c1ncno1]

In some embodiments, at least one R^{T1} is thiadiazolyl.

In some embodiments, at least one R^{T1} is

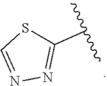 [SMILES: *c1nncs1]

In some embodiments, at least one R^{T1} is

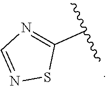 [SMILES: *c1ncns1]

In some embodiments, at least one R^{T1} is 5-membered heteroaryl containing four heteroatoms selected from N, O, and S (e.g., tetrazolyl or oxatriazolyl).

In some embodiments, at least one R^{T1} is 5-membered heteroaryl containing four heteroatoms selected from N, O, and S (e.g., tetrazolyl or oxatriazolyl), wherein the heteroaryl is substituted with one or more R^{Ta}.

In some embodiments, at least one R^{T1} is tetrazolyl.

In some embodiments, at least one R^{T1} is

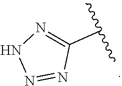 [SMILES: *c1nn[nH]n1]

In some embodiments, at least one R^{T1} is oxatriazolyl.

In some embodiments, at least one R^{T1} is 6-membered heteroaryl containing at least one heteroatom selected from N, O, and S.

In some embodiments, at least one R^{T1} is 6-membered heteroaryl containing at least one heteroatom selected from N, O, and S, wherein the heteroaryl is substituted with one or more R^{Ta}.

In some embodiments, at least one R^{T1} is 6-membered heteroaryl containing one heteroatom selected from N, O, and S (e.g., pyridinyl, pyranyl, or thiopyranyl).

In some embodiments, at least one R^{T1} is 6-membered heteroaryl containing one heteroatom selected from N, O, and S (e.g., pyridinyl, pyranyl, or thiopyranyl), wherein the heteroaryl is substituted with one or more R^{Ta}.

In some embodiments, at least one R^{T1} is pyridinyl.

In some embodiments, at least one R^{T1} is

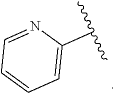 [SMILES: *c1ccccn1]

In some embodiments, at least one R^{T1} is 6-membered heteroaryl containing two heteroatoms selected from N, O, and S (e.g., pyrimidinyl, pyridazinyl, or pyrazinyl).

In some embodiments, at least one R^{T1} is 6-membered heteroaryl containing two heteroatoms selected from N, O, and S (e.g., pyrimidinyl, pyridazinyl, or pyrazinyl), wherein the heteroaryl is substituted with one or more R^{Ta}.

In some embodiments, at least one R^{T1} is pyrimidinyl.

In some embodiments, at least one R^{T1} is

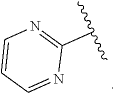 [SMILES: *c1ncccn1]

In some embodiments, at least one R^{T1} is

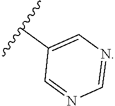 [SMILES: *c1cncnc1]

In some embodiments, at least one R^{T1} is pyridazinyl.

In some embodiments, at least one R^{T1} is

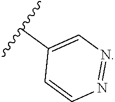 [SMILES: *c1ccnnc1]

In some embodiments, at least one R^{T1} is pyrazinyl.

In some embodiments, at least one R^{T1} is 9-membered heteroaryl containing at least one heteroatom selected from N, O, and S.

In some embodiments, at least one R^{T1} is 9-membered heteroaryl containing at least one heteroatom selected from N, O, and S, wherein the heteroaryl is substituted with one or more R^{Ta}.

In some embodiments, at least one R^{T1} is

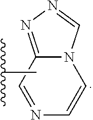

In some embodiments, at least one R^{T1} is

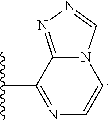 [SMILES: *c1nccn2cnnc12]

In some embodiments, at least one R^{T1} is —(C₁-C₆ alkyl)-(C₃-C₆ cycloalkyl).

In some embodiments, at least one R^{T1} is —(C₁-C₆ alkyl)-(4- to 10-membered heterocyclyl).

In some embodiments, at least one R^{T1} is —(C₁-C₆ alkyl)-(C₆-C₁₀ aryl).

In some embodiments, at least one R^{T1} is —(C₁-C₆ alkyl)-(5- to 10-membered heteroaryl).

In some embodiments, R^{T1} is H or C₁-C₆ alkyl; and the other R^{T1} is C₁-C₆ alkyl, C₂-C₆ alkenyl, C₂-C₆ alkynyl, cyclopropyl, cyclobutyl, cyclopentyl, cyclohexyl, cycloheptyl, cyclooctyl,

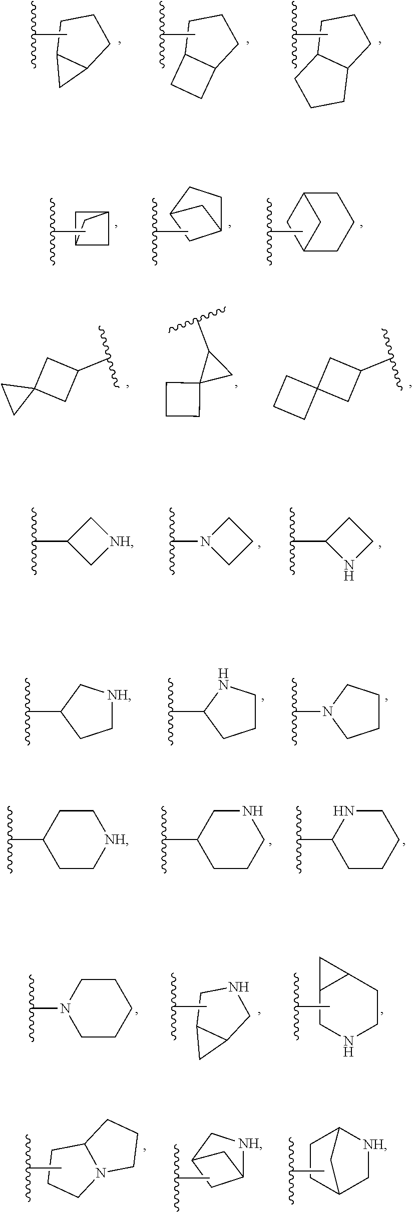

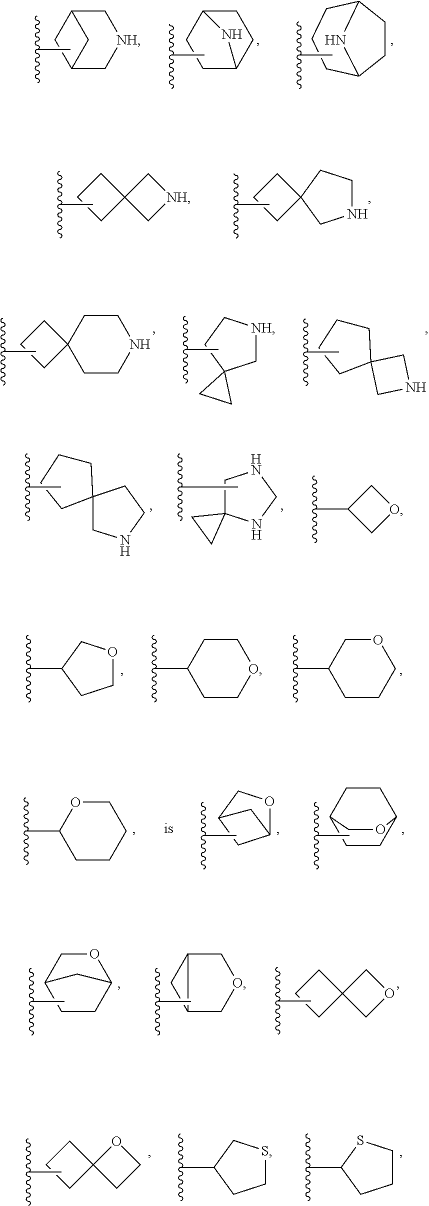

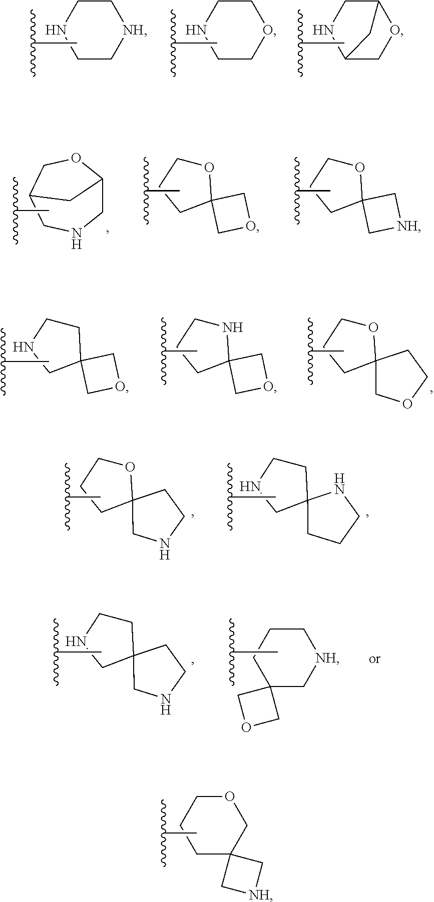

wherein the other R^{T1} is optionally substituted with one or more R^{Ta}.

In some embodiments, R^{T1} is H or C₁-C₆ alkyl; and the other R^{T1} is pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, pyrazinyl, phenyl, or

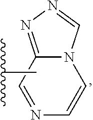

wherein the other R^{T1} is optionally substituted with one or more R^{Ta}.

### Variable R^{T2}

In some embodiments, two R^{T2}, together with the atom to which they are attached, form 4- to 10-membered heterocyclyl optionally substituted with one or more R^{Ta}.

In some embodiments, two R^{T2}, together with the atom to which they are attached, form 4- to 10-membered heterocyclyl.

In some embodiments, two R^{T2}, together with the atom to which they are attached, form 4- to 10-membered heterocyclyl substituted with one or more R^{Ta}.

In some embodiments, two R^{T2}, together with the atom to which they are attached, form a monocyclic 4- to 8-membered heterocyclyl, a fused bicyclic 4- to 10-membered heterocyclyl, a bridged bicyclic 5- to 10-membered heterocyclyl, or a spiro bicyclic 5- to 10-membered heterocyclyl, each optionally substituted with one or more R^{Ta}.

In some embodiments, two R^{T2}, together with the atom to which they are attached, form a monocyclic 4- to 8-membered heterocyclyl, a fused bicyclic 4- to 10-membered heterocyclyl, a bridged bicyclic 6- to 10-membered heterocyclyl, or a spiro bicyclic 6- to 10-membered heterocyclyl, each optionally substituted with one or more R^{Ta}.

In some embodiments, two R^{T2}, together with the atom to which they are attached, a fused bicyclic 6- to 10-membered heterocyclyl or a spiro bicyclic 6- to 10-membered heterocyclyl, each optionally substituted with one or more R^{Ta}.

In some embodiments, two R^{T2}, together with the atom to which they are attached, form 4- to 10-membered monocyclic heterocyclyl.

In some embodiments, two R^{T2}, together with the atom to which they are attached, form 4- to 10-membered monocyclic heterocyclyl substituted with one or more R^{Ta}.

In some embodiments, two R^{T2}, together with the atom to which they are attached, form 4- to 10-membered monocyclic heterocyclyl containing one nitrogen.

In some embodiments, two R^{T2}, together with the atom to which they are attached, form 4- to 10-membered monocyclic heterocyclyl, wherein the heterocyclyl contains one nitrogen and is substituted with one or more R^{Ta}.

In some embodiments, two R^{T2}, together with the atom to which they are attached, form 4- to 10-membered monocyclic heterocyclyl containing two nitrogens.

In some embodiments, two R^{T2}, together with the atom to which they are attached, form 4- to 10-membered monocyclic heterocyclyl, wherein the heterocyclyl contains two nitrogens and is substituted with one or more R^{Ta}.

In some embodiments, two R^{T2}, together with the atom to which they are attached, form 4- to 10-membered monocyclic heterocyclyl containing three nitrogens.

In some embodiments, two R^{T2}, together with the atom to which they are attached, form 4- to 10-membered monocyclic heterocyclyl, wherein the heterocyclyl contains three nitrogens and is substituted with one or more R^{Ta}.

In some embodiments, two R^{T2}, together with the atom to which they are attached, form 4- to 10-membered monocyclic heterocyclyl containing one nitrogen and one oxygen.

In some embodiments, two R^{T2}, together with the atom to which they are attached, form 4- to 10-membered monocyclic heterocyclyl, wherein the heterocyclyl contains one nitrogen and one oxygen, and is substituted with one or more R^{Ta}.

In some embodiments, two R^{T2}, together with the atom to which they are attached, form

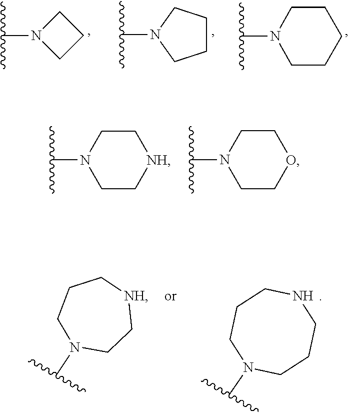

In some embodiments, two R^{T2}, together with the atom to which they are attached, form

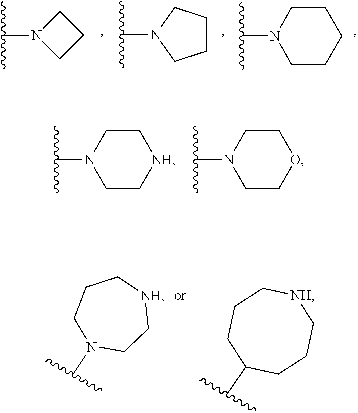

substituted with one or more R^{Ta}.

In some embodiments, two R^{T2}, together with the atom to which they are attached, form 4- to 10-membered bicyclic heterocyclyl.

In some embodiments, two R^{T2}, together with the atom to which they are attached, form 4- to 10-membered bicyclic heterocyclyl substituted with one or more R^{Ta}.

In some embodiments, the 4- to 10-membered bicyclic heterocyclyl formed by two R^{T2} together with the atom to which they are attached, is a 6- to 10-membered bicyclic heterocycle.

In some embodiments, two R^{T2}, together with the atom to which they are attached, form 4- to 10-membered fused bicyclic heterocyclyl.

In some embodiments, two R^{T2}, together with the atom to which they are attached, form 4- to 10-membered fused bicyclic heterocyclyl substituted with one or more R^{Ta}.

In some embodiments, two R^{T2}, together with the atom to which they are attached, form 4- to 10-membered fused bicyclic heterocyclyl containing one nitrogen.

In some embodiments, two R^{T2}, together with the atom to which they are attached, form 4- to 10-membered fused bicyclic heterocyclyl, wherein the heterocyclyl contains one nitrogen and is substituted with one or more R^{Ta}.

In some embodiments, two R^{T2}, together with the atom to which they are attached, form 4- to 10-membered fused bicyclic heterocyclyl containing two nitrogens.

In some embodiments, two R^{T2}, together with the atom to which they are attached, form 4- to 10-membered fused bicyclic heterocyclyl, wherein the heterocyclyl contains two nitrogens and is substituted with one or more R^{Ta}.

In some embodiments, two R^{T2}, together with the atom to which they are attached, form 4- to 10-membered fused bicyclic heterocyclyl containing three nitrogens.

In some embodiments, two R^{T2}, together with the atom to which they are attached, form 4- to 10-membered fused bicyclic heterocyclyl, wherein the heterocyclyl contains three nitrogens and is substituted with one or more R^{Ta}.

In some embodiments, two R^{T2}, together with the atom to which they are attached, form 4- to 10-membered fused bicyclic heterocyclyl containing four nitrogens.

In some embodiments, two R^{T2}, together with the atom to which they are attached, form 4- to 10-membered fused bicyclic heterocyclyl, wherein the heterocyclyl contains four nitrogens and is substituted with one or more R^{Ta}.

In some embodiments, two R^{T2}, together with the atom to which they are attached, form 4- to 10-membered fused bicyclic heterocyclyl containing one nitrogen and one oxygen.

In some embodiments, two R^{T2}, together with the atom to which they are attached, form 4- to 10-membered fused bicyclic heterocyclyl, wherein the heterocyclyl contains one nitrogen and one oxygen, and is substituted with one or more R^{Ta}.

In some embodiments, two R^{T2}, together with the atom to which they are attached, form 4- to 10-membered fused bicyclic heterocyclyl containing two nitrogens and one oxygen.

In some embodiments, two R^{T2}, together with the atom to which they are attached, form 4- to 10-membered fused bicyclic heterocyclyl, wherein the heterocyclyl contains two nitrogens and one oxygen, and is substituted with one or more R^{Ta}.

In some embodiments, two R^{T2}, together with the atom to which they are attached, form

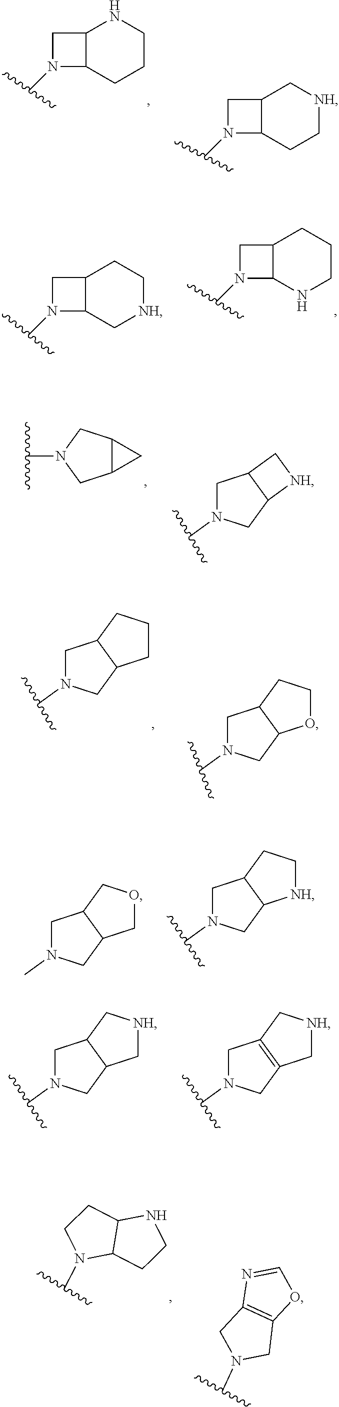

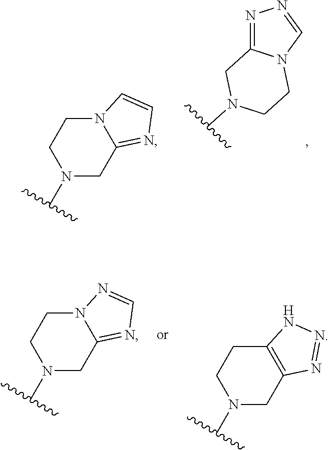 [SMILES: *N1CCn2ccnc2C1 ; *N1CCn2cnnc2C1 ; *N1CCn2ncnc2C1 ; *N1CCc2[nH]nnc2C1]

In some embodiments, two R^{T2} together with the atom to which they are attached, form

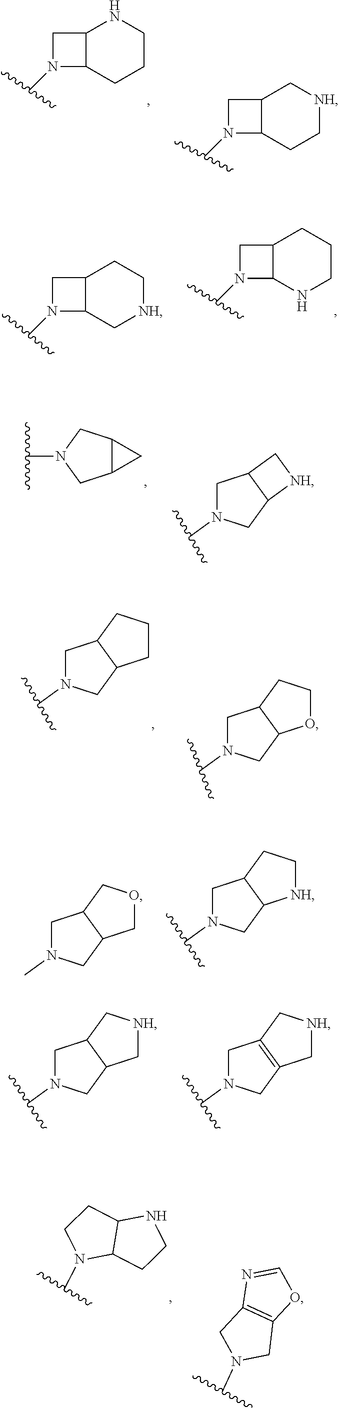

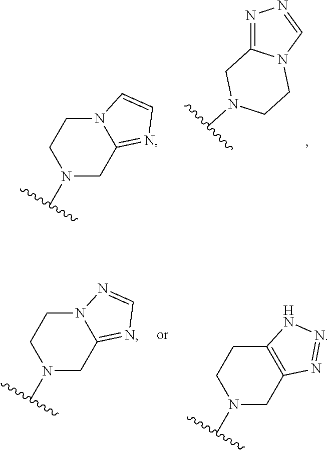 [SMILES: *N1CCn2ccnc2C1 ; *N1CCn2cnnc2C1 ; *N1CCn2ncnc2C1 ; *N1CCc2[nH]nnc2C1]

substituted with one or more R^{Ta}.

In some embodiments, two R^{T2}, together with the atom to which they are attached, form

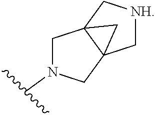 [SMILES: *N1CC23CNCC2(C1)C3]

In some embodiments, two R^{T2}, together with the atom to which they are attached, form

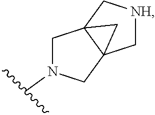 [SMILES: *N1CC23CNCC2(C1)C3]

substituted with one or more R^{Ta}.

In some embodiments, the 4- to 10-membered fused bicyclic heterocyclyl formed by two R^{T2} together with the atom to which they are attached, is a 6- to 10-membered fused bicyclic heterocycle.

In some embodiments, two R^{T2}, together with the atom to which they are attached, form 5- to 10-membered bridged bicyclic heterocyclyl.

In some embodiments, two R^{T2}, together with the atom to which they are attached, form 5- to 10-membered bridged bicyclic heterocyclyl substituted with one or more R^{Ta}.

In some embodiments, two R^{T2}, together with the atom to which they are attached, form 5- to 10-membered bridged bicyclic heterocyclyl containing one nitrogen.

In some embodiments, two R^{T2}, together with the atom to which they are attached, form 5- to 10-membered bridged bicyclic heterocyclyl, wherein the heterocyclyl contains one nitrogen and is substituted with one or more R^{Ta}.

In some embodiments, two R^{T2}, together with the atom to which they are attached, form 5- to 10-membered bridged bicyclic heterocyclyl containing two nitrogens.

In some embodiments, two R^{T2}, together with the atom to which they are attached, form 5- to 10-membered bridged bicyclic heterocyclyl, wherein the heterocyclyl contains two nitrogens and is substituted with one or more R^{Ta}.

In some embodiments, two R^{T2}, together with the atom to which they are attached, form 5- to 10-membered bridged bicyclic heterocyclyl containing one nitrogen and one oxygen.

In some embodiments, two R^{T2}, together with the atom to which they are attached, form 5- to 10-membered bridged bicyclic heterocyclyl, wherein the heterocyclyl contains one nitrogen and one oxygen, and is substituted with one or more R^{Ta}.

In some embodiments, two R^{T2}, together with the atom to which they are attached, form 5- to 10-membered bridged bicyclic heterocyclyl containing two nitrogens and one oxygen.

In some embodiments, two R^{T2}, together with the atom to which they are attached, form 5- to 10-membered bridged bicyclic heterocyclyl, wherein the heterocyclyl contains two nitrogens and one oxygen, and is substituted with one or more R^{Ta}.

In some embodiments, two R^{T2}, together with the atom to which they are attached, form

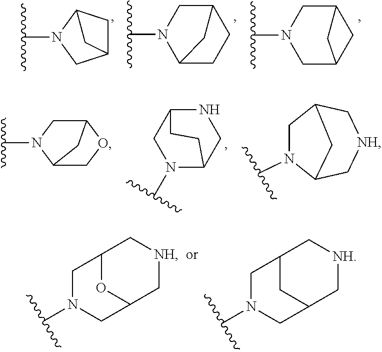

In some embodiments, two R^{T2}, together with the atom to which they are attached, form

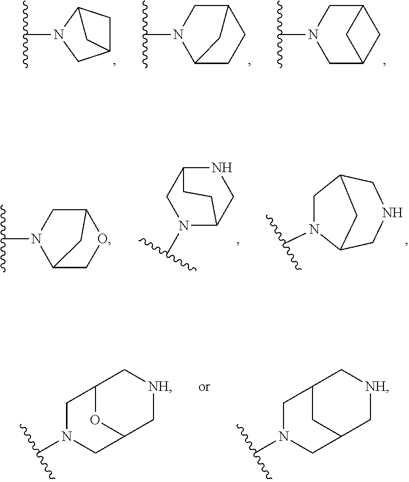

substituted with one or more R^{Ta}.

In some embodiments, the 5- to 10-membered bridged bicyclic heterocyclyl formed by two R^{T2} together with the atom to which they are attached, is a 6- to 10-membered bridged bicyclic heterocycle.

In some embodiments, two R^{T2}, together with the atom to which they are attached, form 5- to 10-membered spiro bicyclic heterocyclyl.

In some embodiments, two R^{T2}, together with the atom to which they are attached, form 5- to 10-membered spiro bicyclic heterocyclyl substituted with one or more R^{Ta}.

In some embodiments, two R^{T2}, together with the atom to which they are attached, form 5- to 10-membered spiro bicyclic heterocyclyl containing one nitrogen.

In some embodiments, two R^{T2}, together with the atom to which they are attached, form 5- to 10-membered spiro bicyclic heterocyclyl, wherein the heterocyclyl contains one nitrogen and is substituted with one or more R^{Ta}.

In some embodiments, two R^{T2}, together with the atom to which they are attached, form 5- to 10-membered spiro bicyclic heterocyclyl containing two nitrogens.

In some embodiments, two R^{T2}, together with the atom to which they are attached, form 5- to 10-membered spiro bicyclic heterocyclyl, wherein the heterocyclyl contains two nitrogens and is substituted with one or more R^{Ta}.

In some embodiments, two R^{T2}, together with the atom to which they are attached, form 5- to 10-membered spiro bicyclic heterocyclyl containing three nitrogens.

In some embodiments, two R^{T2}, together with the atom to which they are attached, form 5- to 10-membered spiro bicyclic heterocyclyl, wherein the heterocyclyl contains three nitrogens and is substituted with one or more R^{Ta}.

In some embodiments, two R^{T2}, together with the atom to which they are attached, form 5- to 10-membered spiro bicyclic heterocyclyl containing one nitrogen and one oxygen.

In some embodiments, two R^{T2}, together with the atom to which they are attached, form 5- to 10-membered spiro bicyclic heterocyclyl, wherein the heterocyclyl contains one nitrogen and one oxygen, and is substituted with one or more R^{Ta}.

In some embodiments, two R^{T2}, together with the atom to which they are attached, form 5- to 10-membered spiro bicyclic heterocyclyl containing two nitrogens and one oxygen.

In some embodiments, two R^{T2}, together with the atom to which they are attached, form 5- to 10-membered spiro bicyclic heterocyclyl, wherein the heterocyclyl contains two nitrogens and one oxygen, and is substituted with one or more R^{Ta}.

In some embodiments, two R^{T2}, together with the atom to which they are attached, form 5- to 10-membered spiro bicyclic heterocyclyl containing one nitrogen and two oxygens.

In some embodiments, two R^{T2}, together with the atom to which they are attached, form 5- to 10-membered spiro bicyclic heterocyclyl, wherein the heterocyclyl contains one nitrogen and two oxygens, and is substituted with one or more R^{Ta}.

In some embodiments, two R^{T2}, together with the atom to which they are attached, form

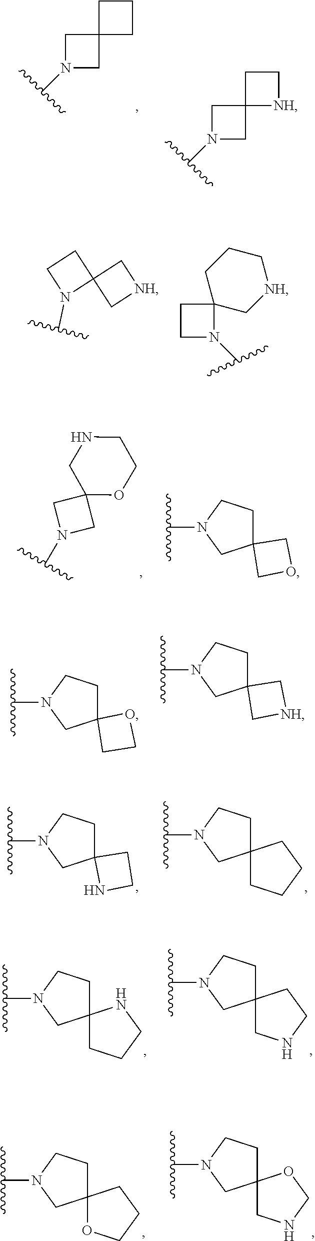

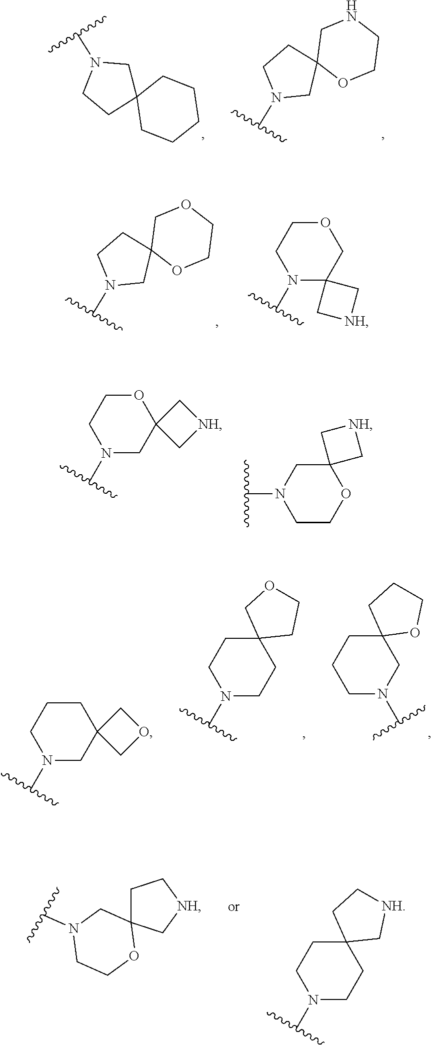

In some embodiments, two R^{T2}, together with the atom to which they are attached, form

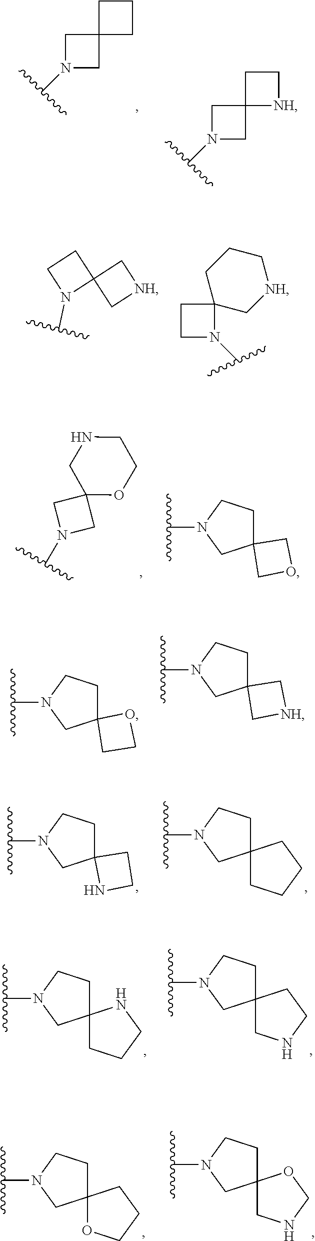

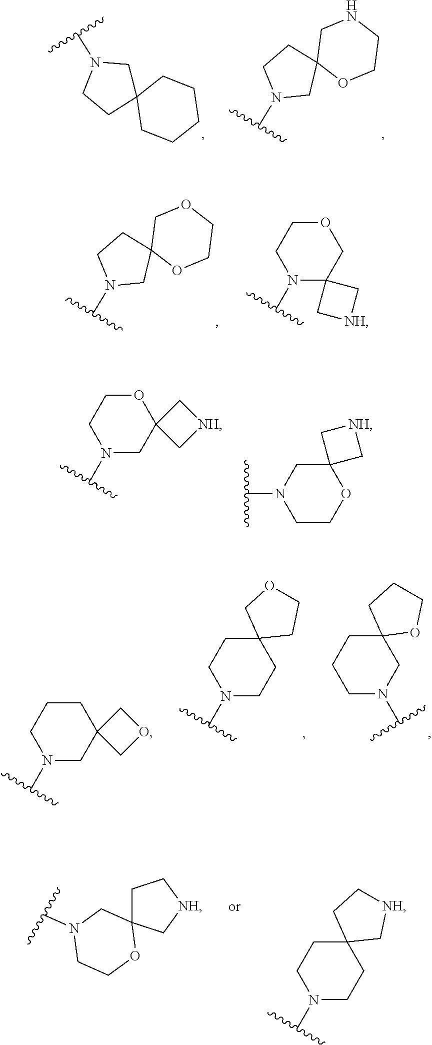

substituted with one or more R^{Ta}.

In some embodiments, the 5- to 10-membered spiro bicyclic heterocyclyl formed by two R^{T2} together with the atom to which they are attached, is a 6- to 10-membered spiro bicyclic heterocycle.

### Variable R^{Ta}

In some embodiments, at least one R^{Ta} is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb}.

In some embodiments, at least one R^{Ta} is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{Ta} is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{Tb}.

In some embodiments, at least one R^{Ta} is oxo.

In some embodiments, at least one R^{Ta} is halogen (e.g., —F, —Cl, —Br, —I).

In some embodiments, at least one R^{Ta} is cyano.

In some embodiments, at least one R^{Ta} is —OH.

In some embodiments, at least one R^{Ta} is —OR^{b}.

In some embodiments, at least one R^{Ta} is —NH₂.

In some embodiments, at least one R^{Ta} is —NHR^{b}.

In some embodiments, at least one R^{Ta} is —N(R^{b})₂.

In some embodiments, at least one R^{Ta} is —C(═O)—H.

In some embodiments, at least one R^{Ta} is —C(═O)—R^{b}.

In some embodiments, at least one R^{Ta} is —C(═O)—OH.

In some embodiments, at least one R^{Ta} is —C(═O)—OR^{b}.

In some embodiments, at least one R^{Ta} is —C(═O)—NH₂.

In some embodiments, at least one R^{Ta} is —C(═O)—NHR^{b}.

In some embodiments, at least one R^{Ta} is —C(═O)—N(R^{b})₂.

In some embodiments, at least one R^{Ta} is —S(═O)₂—R^{b}.

In some embodiments, at least one R^{Ta} is —S(═O)(═NH)—R^{b}.

In some embodiments, at least one R^{Ta} is C₁-C₆ alkyl (e.g., methyl, ethyl, n-propyl, isopropyl, n-butyl, t-butyl, isobutyl, or sec-butyl).

In some embodiments, at least one R^{Ta} is C₁-C₆ alkyl (e.g., methyl, ethyl, n-propyl, isopropyl, n-butyl, t-butyl, isobutyl, or sec-butyl) substituted with one or more R^{Tb}.

In some embodiments, at least one R^{Ta} is methyl.

In some embodiments, at least one R^{Ta} is ethyl.

In some embodiments, at least one R^{Ta} is n-propyl.

In some embodiments, at least one R^{Ta} is isopropyl.

In some embodiments, at least one R^{Ta} is n-butyl.

In some embodiments, at least one R^{Ta} is C₂-C₆ alkenyl.

In some embodiments, at least one R^{Ta} is C₂-C₆ alkenyl substituted with one or more R^{Tb}.

In some embodiments, at least one R^{Ta} is C₂-C₆ alkynyl.

In some embodiments, at least one R^{Ta} is C₂-C₆ alkynyl substituted with one or more R^{Tb}.

In some embodiments, at least one R^{Ta} is C₃-C₆ cycloalkyl.

In some embodiments, at least one R^{Ta} is C₃-C₆ cycloalkyl substituted with one or more R^{Tb}.

In some embodiments, at least one R^{Ta} is C₃-C₆ monocyclic cycloalkyl.

In some embodiments, at least one R^{Ta} is C₃-C₆ monocyclic cycloalkyl substituted with one or more R^{Tb}.

In some embodiments, at least one R^{Ta} is cyclopropyl, cyclobutyl, cyclopentyl, cyclohexyl, cycloheptyl, or cyclooctyl.

In some embodiments, at least one R^{Ta} is C₃-C₆ bicyclic cycloalkyl.

In some embodiments, at least one R^{Ta} is C₃-C₆ bicyclic cycloalkyl substituted with one or more R^{Tb}.

In some embodiments, at least one R^{Ta} is bicyclocyclobutyl, bicyclocyclopentyl, or bicyclocyclohexyl.

In some embodiments, at least one R^{Ta} is C₄-C₆ fused bicyclic cycloalkyl.

In some embodiments, at least one R^{Ta} is C₄-C₆ fused bicyclic cycloalkyl substituted with one or more R^{Tb}.

In some embodiments, at least one R^{Ta} is

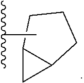

In some embodiments, at least one R^{Ta} is

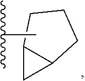

substituted with one or more R^{Tb}.

In some embodiments, at least one R^{Ta} is

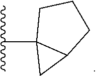 [SMILES: *C12CCCC1C2]

In some embodiments, at least one R^{Ta} is

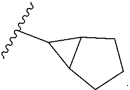 [SMILES: *C1C2CCCC12]

In some embodiments, at least one R^{Ta} is C₅-C₆ bridged bicyclic cycloalkyl.

In some embodiments, at least one R^{Ta} is C₅-C₆ bridged bicyclic cycloalkyl substituted with one or more R^{Tb}.

In some embodiments, at least one R^{Ta} is

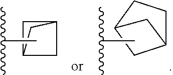

In some embodiments, at least one R^{Ta} is

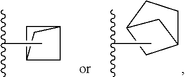

substituted with one or more R^{Tb}.

In some embodiments, at least one R^{Ta} is

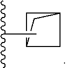

In some embodiments, at least one R^{Ta} is

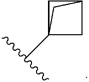 [SMILES: *C12CC(C1)C2]

In some embodiments, at least one R^{Ta} is

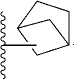

In some embodiments, at least one R^{Ta} is

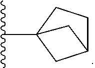 [SMILES: *C12CCC(C1)C2]

In some embodiments, at least one R^{Ta} is C₅-C₆ spiro bicyclic cycloalkyl.

In some embodiments, at least one R^{Ta} is C₅-C₆ spiro bicyclic cycloalkyl substituted with one or more R^{Tb}.

In some embodiments, at least one R^{Ta} is spiro[2.2]pentyl or spiro[2.3]hexyl.

In some embodiments, at least one R^{Ta} is spiro[2.2]pentyl or spiro[2.3]hexyl, substituted with one or more R^{Tb}.

In some embodiments, at least one R^{Ta} is

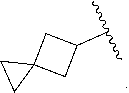 [SMILES: *C1CC2(CC2)C1]

In some embodiments, at least one R^{Ta} is

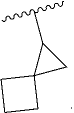 [SMILES: *C1CC12CCC2]

In some embodiments, at least one R^{Ta} is 4- to 10-membered heterocyclyl.

In some embodiments, at least one R^{Ta} is 4- to 10-membered heterocyclyl substituted with one or more R^{Tb}.

In some embodiments, at least one R^{Ta} is 4- to 10-membered monocyclic heterocyclyl.

In some embodiments, at least one R^{Ta} is 4- to 10-membered monocyclic heterocyclyl substituted with one or more R^{Tb}.

In some embodiments, at least one R^{Ta} is 4- to 10-membered bicyclic heterocyclyl.

In some embodiments, at least one R^{Ta} is 4- to 10-membered bicyclic heterocyclyl substituted with one or more R^{Tb}.

In some embodiments, at least one R^{Ta} is 4- to 10-membered fused bicyclic heterocyclyl.

In some embodiments, at least one R^{Ta} is 4- to 10-membered fused bicyclic heterocyclyl substituted with one or more R^{Tb}.

In some embodiments, at least one R^{Ta} is 5- to 10-membered bridged bicyclic heterocyclyl.

In some embodiments, at least one R^{Ta} is 5- to 10-membered bridged bicyclic heterocyclyl substituted with one or more R^{Tb}.

In some embodiments, at least one R^{Ta} is 5- to 10-membered spiro bicyclic heterocyclyl.

In some embodiments, at least one R^{Ta} is 5- to 10-membered spiro bicyclic heterocyclyl substituted with one or more R^{Tb}.

In some embodiments, at least one R^{Ta} is 4- to 10-membered heterocyclyl containing at least one heteroatom selected from N, O, and S.

In some embodiments, at least one R^{Ta} is 4- to 10-membered heterocyclyl containing at least one heteroatom selected from N, O, and S, wherein the 4- to 10-membered heterocyclyl is substituted with one or more R^{Tb}.

In some embodiments, at least one R^{Ta} is 4- to 10-membered monocyclic heterocyclyl containing at least one heteroatom selected from N, O, and S.

In some embodiments, at least one R^{Ta} is 4- to 10-membered monocyclic heterocyclyl containing at least one heteroatom selected from N, O, and S, wherein the 4- to 10-membered heterocyclyl is substituted with one or more R^{Tb}.

In some embodiments, at least one R^{Ta} is 4- to 10-membered monocyclic heterocyclyl containing one N (e.g., azetidinyl, pyrrolidinyl, or piperidinyl).

In some embodiments, at least one R^{Ta} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Tb}.

In some embodiments, at least one R^{Ta} is azetidinyl.

In some embodiments, at least one R^{Ta} is azetidinyl substituted with one or more R^{Tb}.

In some embodiments, at least one R^{Ta} is

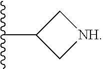 [SMILES: *C1CNC1]

In some embodiments, at least one R^{Ta} is

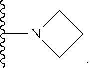 [SMILES: *N1CCC1]

In some embodiments, at least one R^{Ta} is

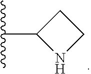 [SMILES: *C1CCN1]

In some embodiments, at least one R^{Ta} is pyrrolidinyl.

In some embodiments, at least one R^{Ta} is

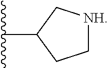 [SMILES: *C1CCNC1]

In some embodiments, at least one R^{Ta} is

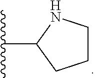 [SMILES: *C1CCCN1]

In some embodiments, at least one R^{Ta} is

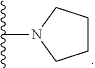 [SMILES: *N1CCCC1]

In some embodiments, at least one R^{Ta} is piperidinyl.

In some embodiments, at least one R^{Ta} is

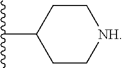 [SMILES: *C1CCNCC1]

In some embodiments, at least one R^{Ta} is

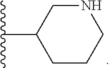 [SMILES: *C1CCCNC1]

In some embodiments, at least one R^{Ta} is

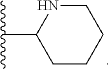 [SMILES: *C1CCCCN1]

In some embodiments, at least one R^{Ta} is

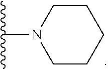 [SMILES: *N1CCCCC1]

In some embodiments, at least one R^{Ta} is 4- to 10-membered fused bicyclic heterocyclyl containing one N.

In some embodiments, at least one R^{Ta} is 4- to 10-membered fused bicyclic heterocyclyl containing one N, wherein the 4- to 10-membered fused bicyclic heterocyclyl is substituted with one or more R^{Tb}.

In some embodiments, at least one R^{Ta} is

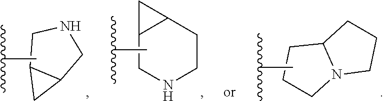

In some embodiments, at least one R^{Ta} is

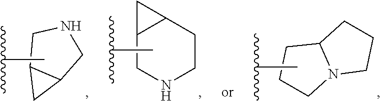

substituted with one or more R^{Tb}.

In some embodiments, at least one R^{Ta} is

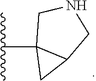 [SMILES: *C12CNCC1C2]

In some embodiments, at least one R^{Ta} is

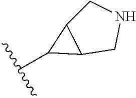 [SMILES: *C1C2CNCC12]

In some embodiments, at least one R^{Ta} is

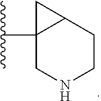 [SMILES: *C12CNCCC1C2]

In some embodiments, at least one R^{Ta} is

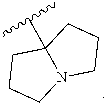 [SMILES: *C12CCCN1CCC2]

In some embodiments, at least one R^{Ta} is 5- to 10-membered bridged bicyclic heterocyclyl containing one N.

In some embodiments, at least one R^{Ta} is 5- to 10-membered bridged bicyclic heterocyclyl containing one N, wherein the 5- to 10-membered bridged bicyclic heterocyclyl is substituted with one or more R^{Tb}.

In some embodiments, at least one R^{Ta} is

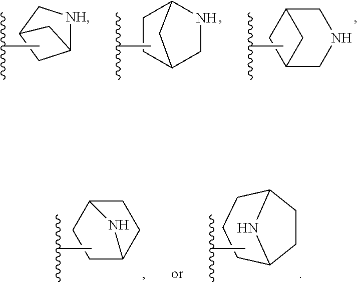

In some embodiments, at least one R^{Ta} is

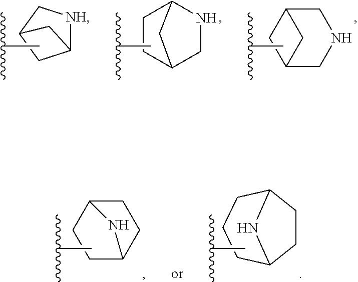

substituted with one or more R^{Tb}.

In some embodiments, at least one R^{Ta} is

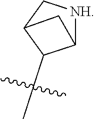 [SMILES: *C1C2CNC1C2]

In some embodiments, at least one R^{Ta} is

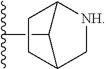 [SMILES: *C1C2CCC1NC2]

In some embodiments, at least one R^{Ta} is

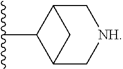 [SMILES: *C1C2CNCC1C2]

In some embodiments, at least one R^{Ta} is

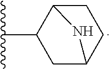 [SMILES: *C1CC2CCC1N2]

In some embodiments, at least one R^{Ta} is

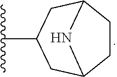 [SMILES: *C1CC2CCC(C1)N2]

In some embodiments, at least one R^{Ta} is

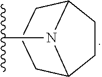 [SMILES: *N1C2CCCC1CC2]

In some embodiments, at least one R^{Ta} is 5- to 10-membered spiro bicyclic heterocyclyl containing one N.

In some embodiments, at least one R^{Ta} is 5- to 10-membered spiro bicyclic heterocyclyl containing one N, wherein the 5- to 10-membered spiro bicyclic heterocyclyl is substituted with one or more R.

In some embodiments, at least one R^{Ta} is

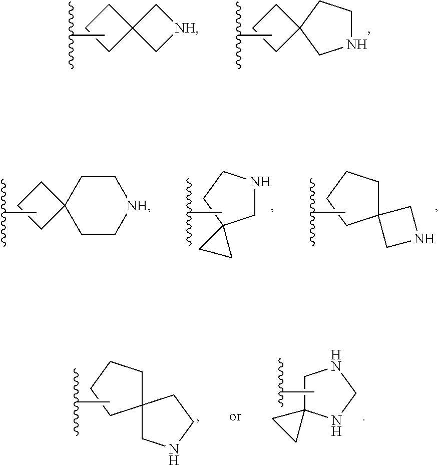

In some embodiments at least one R^{Ta} is

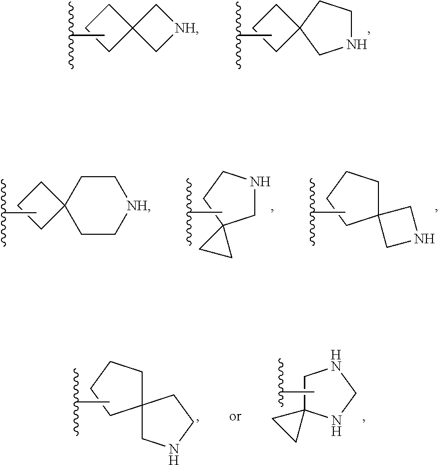

substituted with one or more R^{Tb}.

In some embodiments, at least one R^{Ta} is

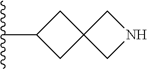 [SMILES: *C1CC2(CNC2)C1]

In some embodiments, at least one R^{Ta} is

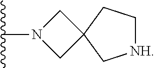 [SMILES: *N1CC2(CCNC2)C1]

In some embodiments, at least one R^{Ta} is

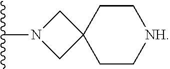 [SMILES: *N1CC2(CCNCC2)C1]

In some embodiments, at least one R^{Ta} is

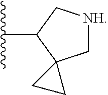 [SMILES: *C1CNCC12CC2]

In some embodiments, at least one R^{Ta} is

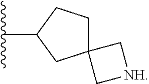 [SMILES: *C1CCC2(CNC2)C1]

In some embodiments, at least one R^{Ta} is

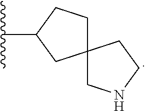 [SMILES: *C1CCC2(CCNC2)C1]

In some embodiments, at least one R^{Ta} is 4- to 10-membered monocyclic heterocyclyl containing one O (e.g., oxetanyl, tetrahydrofuranyl, or tetrahydropyranyl).

In some embodiments, at least one R^{Ta} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{T}e.

In some embodiments, at least one R^{Ta} is oxetanyl.

In some embodiments, at least one R^{Ta} is

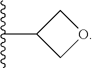 [SMILES: *C1COC1]

In some embodiments, at least one R^{Ta} is

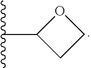 [SMILES: *C1CCO1]

In some embodiments, at least one R^{Ta} is tetrahydrofuranyl.

In some embodiments, at least one R^{Ta} is

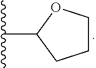 [SMILES: *C1CCCO1]

In some embodiments, at least one R^{Ta} is

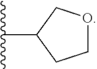 [SMILES: *C1CCOC1]

In some embodiments, at least one R^{Ta} is tetrahydropyranyl.

In some embodiments, at least one R^{Ta} is

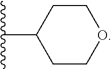 [SMILES: *C1CCOCC1]

In some embodiments, at least one R^{Ta} is

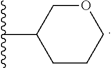 [SMILES: *C1CCCOC1]

In some embodiments, at least one R^{Ta} is

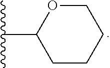 [SMILES: *C1CCCCO1]

In some embodiments, at least one R^{Ta} is 5- to 10-membered bridged bicyclic heterocyclyl containing one O.

In some embodiments, at least one R^{Ta} is 5- to 10-membered bridged bicyclic heterocyclyl containing one O, wherein the 5- to 10-membered bridged bicyclic heterocyclyl is substituted with one or more R^{Tb}.

In some embodiments, at least one R^{Ta} is 4- to 10-membered fused bicyclic heterocyclyl containing one O.

In some embodiments, at least one R^{Ta} is 4- to 10-membered fused bicyclic heterocyclyl containing one O, wherein the 4- to 10-membered fused heterocyclyl is substituted with one or more R^{Tb}.

In some embodiments, at least one R^{Ta} is 5- to 10-membered spiro bicyclic heterocyclyl containing one O.

In some embodiments, at least one R^{Ta} is 5- to 10-membered spiro bicyclic heterocyclyl containing one O, wherein the 5- to 10-membered spiro heterocyclyl is substituted with one or more R^{Tb}.

In some embodiments, at least one R^{Ta} is

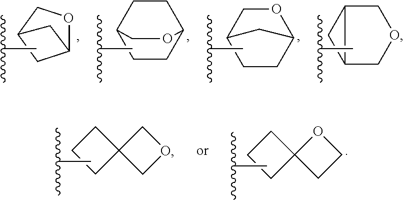

In some embodiments, at least one R^{Ta} is

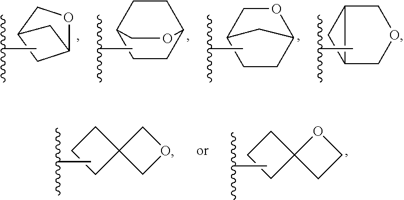

or substituted with one or more R^{Tb}.

In some embodiments, at least one R^{Ta} is

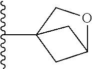 [SMILES: *C12COC(C1)C2]

In some embodiments, at least one R^{Ta} is

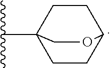 [SMILES: *C12CCC(CC1)OC2]

In some embodiments, at least one R^{Ta} is

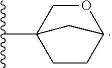 [SMILES: *C12CCC(C1)OC2]

In some embodiments, at least one R^{Ta} is

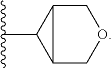 [SMILES: *C1C2COCC12]

In some embodiments, at least one R^{Ta} is

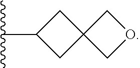 [SMILES: *C1CC2(COC2)C1]

In some embodiments, at least one R^{Ta} is

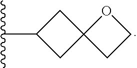 [SMILES: *C1CC2(CCO2)C1]

In some embodiments, at least one R^{Ta} is 4- to 10-membered monocyclic heterocyclyl containing one S (e.g., tetrahydrothiophenyl).

In some embodiments, at least one R^{Ta} is 4- to 10-membered monocyclic heterocyclyl containing one S (e.g., tetrahydrothiophenyl), wherein the 4- to 10-membered monocyclic heterocyclyl is substituted with one or more R^{Tb}.

In some embodiments, at least one R^{Ta} is tetrahydrothiophenyl.

In some embodiments, at least one R^{Ta} is

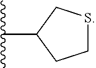 [SMILES: *C1CCSC1]

In some embodiments, at least one R^{Ta} is

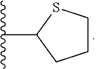 [SMILES: *C1CCCS1]

In some embodiments, at least one R^{Ta} is 4- to 10-membered heterocyclyl (e.g., monocyclic, fused bicyclic, bridged bicyclic, or spiro bicyclic) containing two heteroatoms selected from N, O, and S.

In some embodiments, at least one R^{Ta} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Tb}.

In some embodiments, at least one R^{Ta} is 4- to 10-membered monocyclic heterocyclyl containing two heteroatoms selected from N, O, and S.

In some embodiments, at least one R^{Ta} is 4- to 10-membered monocyclic heterocyclyl containing two heteroatoms selected from N, O, and S, wherein the 4- to 10-membered monocyclic heterocyclyl is substituted with one or more R^{Tb}.

In some embodiments, at least one R^{Ta} is 4- to 10-membered fused bicyclic heterocyclyl containing two heteroatoms selected from N, O, and S.

In some embodiments, at least one R^{Ta} is 4- to 10-membered fused bicyclic heterocyclyl containing two heteroatoms selected from N, O, and S, wherein the 4- to 10-membered fused bicyclic heterocyclyl is substituted with one or more R^{Tb}.

In some embodiments, at least one R^{Ta} is 5- to 10-membered bridged bicyclic heterocyclyl containing two heteroatoms selected from N, O, and S.

In some embodiments, at least one R^{Ta} is 5- to 10-membered bridged bicyclic heterocyclyl containing two heteroatoms selected from N, O, and S, wherein the 5- to 10-membered bridged bicyclic heterocyclyl is substituted with one or more R^{Tb}.

In some embodiments, at least one R^{Ta} is 5- to 10-membered spiro bicyclic heterocyclyl containing two heteroatoms selected from N, O, and S.

In some embodiments, at least one R^{Ta} is 5- to 10-membered spiro bicyclic heterocyclyl containing two heteroatoms selected from N, O, and S, wherein the 5- to 10-membered spiro bicyclic heterocyclyl is substituted with one or more R^{Tb}.

In some embodiments, at least one R^{Ta} is

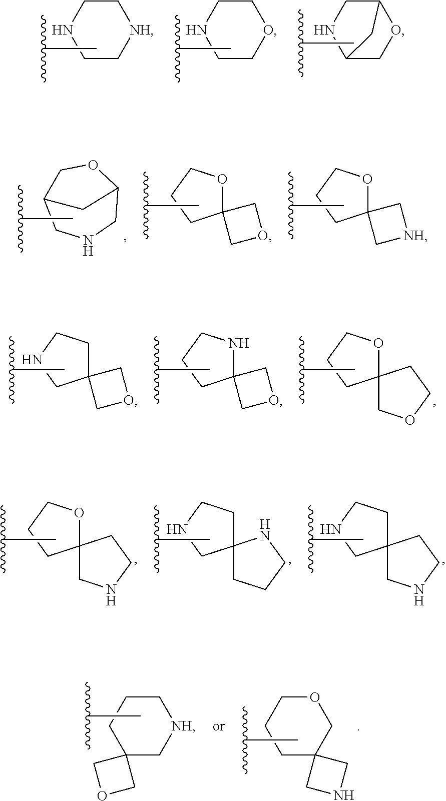

In some embodiments, at least one R^{Ta} is

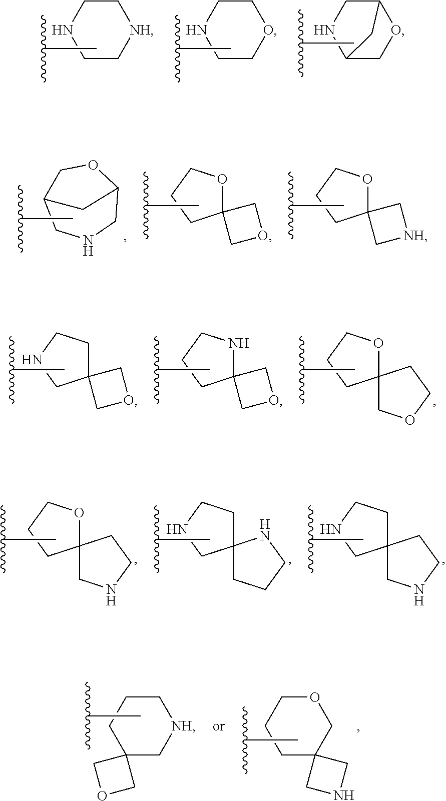

substituted with one or more R^{Tb}.

In some embodiments, at least one R^{Ta} is

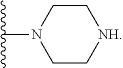 [SMILES: *N1CCNCC1]

In some embodiments, at least one R^{Ta} is

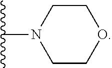 [SMILES: *N1CCOCC1]

In some embodiments, at least one R^{Ta} is

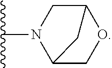 [SMILES: *N1CC2CC1CO2]

In some embodiments, at least one R^{Ta} is

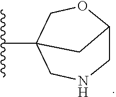 [SMILES: *C12CNCC(C1)OC2]

In some embodiments, at least one R^{Ta} is

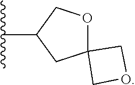 [SMILES: *C1COC2(COC2)C1]

In some embodiments, at least one R^{Ta} is

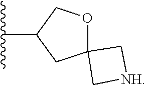 [SMILES: *C1COC2(CNC2)C1]

In some embodiments, at least one R^{Ta} is

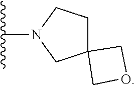 [SMILES: *N1CCC2(COC2)C1]

In some embodiments, at least one R^{Ta} is

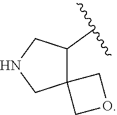 [SMILES: *C1CNCC12COC2]

In some embodiments, at least one R^{Ta} is

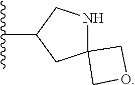 [SMILES: *C1CNC2(COC2)C1]

In some embodiments, at least one R^{Ta} is

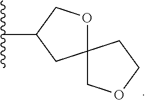 [SMILES: *C1COC2(CCOC2)C1]

In some embodiments, at least one R^{Ta} is

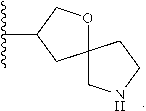 [SMILES: *C1COC2(CCNC2)C1]

In some embodiments, at least one R^{Ta} is

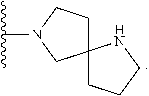 [SMILES: *N1CCC2(CCCN2)C1]

In some embodiments, at least one R^{Ta} is

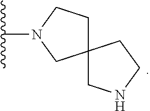 [SMILES: *N1CCC2(CCNC2)C1]

In some embodiments, at least one R^{Ta} is

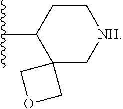 [SMILES: *C1CCNCC12COC2]

In some embodiments, at least one R^{Ta} is

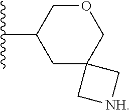 [SMILES: *C1COCC2(CNC2)C1]

In some embodiments, at least one R^{Ta} is C₆-C₁₀ aryl (e.g., phenyl).

In some embodiments, at least one R^{Ta} is C₆-C₁₀ aryl (e.g., phenyl) substituted with one or more R^{Tb}.

In some embodiments, at least one R^{Ta} is phenyl.

In some embodiments, at least one R^{Ta} is 5- to 10-membered heteroaryl. In some embodiments, at least one R^{Ta} is 5- or 6-membered heteroaryl.

In some embodiments, at least one R^{Ta} is 5- to 10-membered heteroaryl substituted with one or more R^{Tb}. In some embodiments, at least one R^{Ta} is 5- or 6-membered heteroaryl substituted with one or more R^{Tb}.

In some embodiments, at least one R^{Ta} is 5- to 10-membered heteroaryl containing at least one heteroatom selected from N, O, and S. In some embodiments, at least one R^{Ta} is 5- or 6-membered heteroaryl containing at least one heteroatom selected from N, O, and S.

In some embodiments, at least one R^{Ta} is 5- to 10-membered heteroaryl containing at least one heteroatom selected from N, O, and S, wherein the heteroaryl is substituted with one or more R^{Tb}. In some embodiments, at least one R^{Ta} is 5- or 6-membered heteroaryl containing at least one heteroatom selected from N, O, and S, wherein the heteroaryl is substituted with one or more R^{Tb}.

In some embodiments, at least one R^{Ta} is 5-membered heteroaryl containing at least one heteroatom selected from N, O, and S.

In some embodiments, at least one R^{Ta} is 5-membered heteroaryl containing at least one heteroatom selected from N, O, and S, wherein the heteroaryl is substituted with one or more R^{Tb}.

In some embodiments, at least one R^{Ta} is 5-membered heteroaryl containing one heteroatom selected from N, O, and S (e.g., pyrrolyl, furanyl, or thiophenyl).

In some embodiments, at least one R^{Ta} is 5-membered heteroaryl containing one heteroatom selected from N, O, and S (e.g., pyrrolyl, furanyl, or thiophenyl), wherein the heteroaryl is substituted with one or more R^{Tb}.

In some embodiments, at least one R^{Ta} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{Ta} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Tb}.

In some embodiments, at least one R^{Ta} is oxazolyl.

In some embodiments, at least one R^{Ta} is

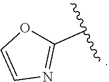 [SMILES: *c1ncco1]

In some embodiments, at least one R^{Ta} is thiazolyl.

In some embodiments, at least one R^{Ta} is thiazolyl.

In some embodiments, at least one R^{Ta} is

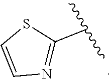 [SMILES: *c1nccs1]

In some embodiments, at least one R^{Ta} is pyrazolyl.

In some embodiments, at least one R^{Ta} is

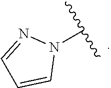 [SMILES: *n1cccn1]

In some embodiments, at least one R^{Ta} is

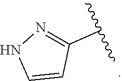 [SMILES: *c1cc[nH]n1]

In some embodiments, at least one R^{Ta} is isoxazolyl.

In some embodiments, at least one R^{Ta} is isothiazolyl.

In some embodiments, at least one R^{Ta} is imidazolyl.

In some embodiments, at least one R^{Ta} is 5-membered heteroaryl containing three heteroatoms selected from N, O, and S (e.g., triazolyl, oxadiazolyl, thiadiazolyl, or oxathiadiazolyl).

In some embodiments, at least one R^{Ta} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Tb}.

In some embodiments, at least one R^{Ta} is triazolyl.

In some embodiments, at least one R^{T}a is

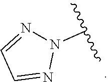 [SMILES: *n1nccn1]

In some embodiments, at least one R^{Ta} is

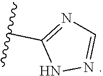 [SMILES: *c1ncn[nH]1]

In some embodiments, at least one R^{Ta} is

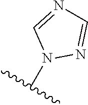 [SMILES: *n1cncn1]

In some embodiments, at least one R^{Ta} is oxadiazolyl.

In some embodiments, at least one R^{Ta} is

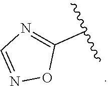 [SMILES: *c1ncno1]

In some embodiments, at least one R^{Ta} is thiadiazolyl.

In some embodiments, at least one R^{Ta} is

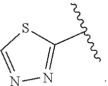 [SMILES: *c1nncs1]

In some embodiments, at least one R^{Ta} is

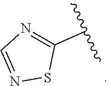 [SMILES: *c1ncns1]

In some embodiments, at least one R^{Ta} is 5-membered heteroaryl containing four heteroatoms selected from N, O, and S (e.g., tetrazolyl or oxatriazolyl).

In some embodiments, at least one R^{Ta} is 5-membered heteroaryl containing four heteroatoms selected from N, O, and S (e.g., tetrazolyl or oxatriazolyl), wherein the heteroaryl is substituted with one or more R^{Tb}.

In some embodiments, at least one R^{Ta} is tetrazolyl.

In some embodiments, at least one R^{Ta} is

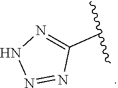 [SMILES: *c1nn[nH]n1]

In some embodiments, at least one R^{Ta} is oxatriazolyl.

In some embodiments, at least one R^{Ta} is 6-membered heteroaryl containing at least one heteroatom selected from N, O, and S.

In some embodiments, at least one R^{Ta} is 6-membered heteroaryl containing at least one heteroatom selected from N, O, and S, wherein the heteroaryl is substituted with one or more R^{Tb}.

In some embodiments, at least one R^{Ta} is 6-membered heteroaryl containing one heteroatom selected from N, O, and S (e.g., pyridinyl, pyranyl, or thiopyranyl).

In some embodiments, at least one R^{Ta} is 6-membered heteroaryl containing one heteroatom selected from N, O, and S (e.g., pyridinyl, pyranyl, or thiopyranyl), wherein the heteroaryl is substituted with one or more R^{Tb}.

In some embodiments, at least one R^{Ta} is pyridinyl, wherein the pyridinyl is substituted with one or more R^{Tb}. In some embodiments, at least one R^{Ta} is pyridinyl. In some embodiments, pyridinyl is 2-pyridinyl, 3-pyridinyl, or 4-pyridinyl.

In some embodiments, at least one R^{Ta} is

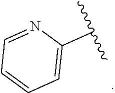 [SMILES: *c1ccccn1]

In some embodiments, at least one R^{Ta} is 6-membered heteroaryl containing two heteroatoms selected from N, O, and S (e.g., pyrimidinyl, pyridazinyl, or pyrazinyl).

In some embodiments, at least one R^{Ta} is 6-membered heteroaryl containing two heteroatoms selected from N, O, and S (e.g., pyrimidinyl, pyridazinyl, or pyrazinyl), wherein the heteroaryl is substituted with one or more R^{Tb}.

In some embodiments, at least one R^{Ta} is pyrimidinyl.

In some embodiments, at least one R^{Ta} is

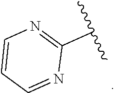 [SMILES: *c1ncccn1]

In some embodiments, at least one R^{Ta} is

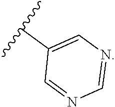 [SMILES: *c1cncnc1]

In some embodiments, at least one R^{Ta} is pyridazinyl.

In some embodiments, at least one R^{Ta} is

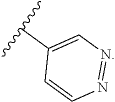 [SMILES: *c1ccnnc1]

In some embodiments, at least one R^{Ta} is pyrazinyl.

In some embodiments, at least one R^{Ta} is 9-membered heteroaryl containing at least one heteroatom selected from N, O, and S.

In some embodiments, at least one R^{Ta} is 9-membered heteroaryl containing at least one heteroatom selected from N, O, and S, wherein the heteroaryl is substituted with one or more R^{Tb}.

In some embodiments, at least one R^{Ta} is

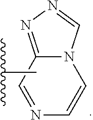

In some embodiments, at least one R^{Ta} is

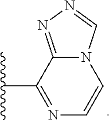 [SMILES: *c1nccn2cnnc12]

### Variable R^{Tb}

In some embodiments, at least one R^{Tb} is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc}.

In some embodiments, at least one R^{Tb} is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{Tc}.

In some embodiments, at least one R^{Tb} is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{Tb} is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, or C₂-C₆ alkynyl.

In some embodiments, at least one R^{Tb} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—(C₁-C₆ alkyl), —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, —S(═O)₂—(C₁-C₆ alkyl), —S(═O)(═NH)—(C₁-C₆ alkyl), C₁-C₆ alkyl, C₂-C₆ alkenyl, or C₂-C₆ alkynyl.

In some embodiments, at least one R^{Tb} is oxo.

In some embodiments, at least one R^{Tb} is halogen (e.g., —F, —Cl, —Br, —I).

In some embodiments, at least one R^{Tb} is cyano.

In some embodiments, at least one R^{Tb} is —OH.

In some embodiments, at least one R^{Tb} is —OR^{c}.

In some embodiments, at least one R^{Tb} is —NH₂.

In some embodiments, at least one R^{Tb} is —NHR^{c}.

In some embodiments, at least one R^{Tb} is —N(R^{c})₂.

In some embodiments, at least one R^{Tb} is —C(═O)—H.

In some embodiments, at least one R^{Tb} is —C(═O)—R^{c}.

In some embodiments, at least one R^{Tb} is —C(═O)—OH.

In some embodiments, at least one R^{Tb} is —C(═O)—OR^{c}.

In some embodiments, at least one R^{Tb} is —C(═O)—NH₂.

In some embodiments, at least one R^{Tb} is —C(═O)—NHR^{c}.

In some embodiments, at least one R^{Tb} is —C(═O)—N(R^{c})₂.

In some embodiments, at least one R^{Tb} is —S(═O)₂—R^{c}.

In some embodiments, at least one R^{Tb} is —S(═O)(═NH)—R^{c}.

In some embodiments, at least one R^{Tb} is C₁-C₆ alkyl (e.g., methyl, ethyl, n-propyl, isopropyl, n-butyl, t-butyl, isobutyl, or sec-butyl).

In some embodiments, at least one R^{Tb} is C₁-C₆ alkyl (e.g., methyl, ethyl, n-propyl, isopropyl, n-butyl, t-butyl, isobutyl, or sec-butyl) substituted with one or more R^{Tc}.

In some embodiments, at least one R^{Tb} is methyl.

In some embodiments, at least one R^{Tb} is ethyl.

In some embodiments, at least one R^{Tb} is n-propyl.

In some embodiments, at least one R^{Tb} is isopropyl.

In some embodiments, at least one R^{Tb} is n-butyl.

In some embodiments, at least one R^{Tb} is C₂-C₆ alkenyl.

In some embodiments, at least one R^{Tb} is C₂-C₆ alkenyl substituted with one or more R^{Tc}.

In some embodiments, at least one R^{Tb} is C₂-C₆ alkynyl.

In some embodiments, at least one R^{Tb} is C₂-C₆ alkynyl substituted with one or more R^{Tc}.

In some embodiments, at least one R^{Tb} is C₃-C₆ cycloalkyl.

In some embodiments, at least one R^{Tb} is 4- to 10-membered heterocyclyl.

In some embodiments, at least one R^{Tb} is C₆-C₁₀ aryl.

In some embodiments, at least one R^{Tb} is 5- to 10-membered heteroaryl.

In some embodiments, at least one R^{Tb} is C₃-C₆ cycloalkyl substituted with one or more R^{Tc}.

In some embodiments, at least one R^{Tb} is C₃-C₆ monocyclic cycloalkyl.

In some embodiments, at least one R^{Tb} is C₃-C₆ monocyclic cycloalkyl substituted with one or more R^{Tc}.

In some embodiments, at least one R^{Tb} is cyclopropyl, cyclobutyl, cyclopentyl, cyclohexyl, cycloheptyl, or cyclooctyl.

In some embodiments, at least one R^{Tb} is C₃-C₆ bicyclic cycloalkyl.

In some embodiments, at least one R^{Tb} is C₃-C₆ bicyclic cycloalkyl substituted with one or more R^{Tc}.

In some embodiments, at least one R^{Tb} is bicyclocyclobutyl, bicyclocyclopentyl, or bicyclocyclohexyl.

In some embodiments, at least one R^{Tb} is C₄-C₆ fused bicyclic cycloalkyl.

In some embodiments, at least one R^{Tb} is C₄-C₆ fused bicyclic cycloalkyl substituted with one or more R^{Tc}.

In some embodiments, at least one R^{Tb} is

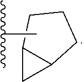

In some embodiments, at least one R^{Tb} is

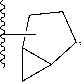

substituted with one or more R^{Tc}.

In some embodiments, at least one R^{Tb} is

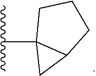 [SMILES: *C12CCCC1C2]

In some embodiments, at least one R^{Tb} is

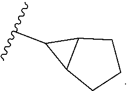 [SMILES: *C1C2CCCC12]

In some embodiments, at least one R^{Tb} is C₅-C₆ bridged bicyclic cycloalkyl.

In some embodiments, at least one R^{Tb} is C₅-C₆ bridged bicyclic cycloalkyl substituted with one or more R^{Tc}.

In some embodiments, at least one R^{Tb} is

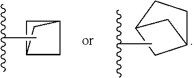

In some embodiments, at least one R^{Tb} is

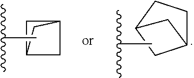

substituted with one or more R^{Tc}.

In some embodiments, at least one R^{Tb} is

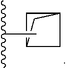

In some embodiments, at least one R^{Tb} is

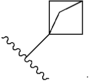 [SMILES: *C12CC(C1)C2]

In some embodiments, at least one R^{Tb} is

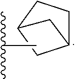

In some embodiments, at least one R^{Tb} is

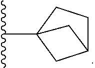 [SMILES: *C12CCC(C1)C2]

In some embodiments, at least one R^{Tb} is C₅-C₆ spiro bicyclic cycloalkyl.

In some embodiments, at least one R^{Tb} is C₅-C₆ spiro bicyclic cycloalkyl substituted with one or more R^{Tc}.

In some embodiments, at least one R^{Tb} is spiro[2.2]pentyl or spiro[2.3]hexyl.

In some embodiments, at least one R^{Tb} is spiro[2.2]pentyl or spiro[2.3]hexyl, substituted with one or more R^{Tc}.

In some embodiments, at least one R^{Tb} is

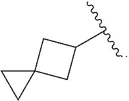 [SMILES: *C1CC2(CC2)C1]

In some embodiments, at least one R^{Tb} is

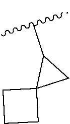 [SMILES: *C1CC12CCC2]

In some embodiments, at least one R^{Tb} is 4- to 10-membered heterocyclyl.

In some embodiments, at least one R^{Tb} is 4- to 10-membered heterocyclyl substituted with one or more R^{Tc}.

In some embodiments, at least one R^{Tb} is 4- to 10-membered monocyclic heterocyclyl.

In some embodiments, at least one R^{Tb} is 4- to 10-membered monocyclic heterocyclyl substituted with one or more R^{Tc}.

In some embodiments, at least one R^{Tb} is 4- to 10-membered bicyclic heterocyclyl.

In some embodiments, at least one R^{Tb} is 4- to 10-membered bicyclic heterocyclyl substituted with one or more R^{Tc}.

In some embodiments, at least one R^{Tb} is 4- to 10-membered fused bicyclic heterocyclyl.

In some embodiments, at least one R^{Tb} is 4- to 10-membered fused bicyclic heterocyclyl substituted with one or more R^{Tc}.

In some embodiments, at least one R^{Tb} is 5- to 10-membered bridged bicyclic heterocyclyl.

In some embodiments, at least one R^{Tb} is 5- to 10-membered bridged bicyclic heterocyclyl substituted with one or more R^{Tc}.

In some embodiments, at least one R^{Tb} is 5- to 10-membered spiro bicyclic heterocyclyl.

In some embodiments, at least one R^{Tb} is 5- to 10-membered spiro bicyclic heterocyclyl substituted with one or more R^{Tc}.

In some embodiments, at least one R^{Tb} is 4- to 10-membered heterocyclyl containing at least one heteroatom selected from N, O, and S.

In some embodiments, at least one R^{Tb} is 4- to 10-membered heterocyclyl containing at least one heteroatom selected from N, O, and S, wherein the 4- to 10-membered heterocyclyl is substituted with one or more R^{Tc}.

In some embodiments, at least one R^{Tb} is 4- to 10-membered monocyclic heterocyclyl containing at least one heteroatom selected from N, O, and S.

In some embodiments, at least one R^{Tb} is 4- to 10-membered monocyclic heterocyclyl containing at least one heteroatom selected from N, O, and S, wherein the 4- to 10-membered heterocyclyl is substituted with one or more R^{Tc}.

In some embodiments, at least one R^{Tb} is 4- to 10-membered monocyclic heterocyclyl containing one N (e.g., azetidinyl, pyrrolidinyl, or piperidinyl).

In some embodiments, at least one R^{Tb} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Tc}.

In some embodiments, at least one R^{Tb} is azetidinyl.

In some embodiments, at least one R^{Tb} is azetidinyl substituted with one or more R^{Tc}.

In some embodiments, at least one R^{Tb} is

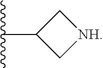 [SMILES: *C1CNC1]

In some embodiments, at least one R^{Tb} is

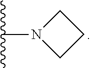 [SMILES: *N1CCC1]

In some embodiments, at least one R^{Tb} is

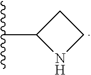 [SMILES: *C1CCN1]

In some embodiments, at least one R^{Tb} is pyrrolidinyl.

In some embodiments, at least one R^{Tb} is

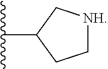 [SMILES: *C1CCNC1]

In some embodiments, at least one R^{Tb} is

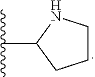 [SMILES: *C1CCCN1]

In some embodiments, at least one R^{Tb} is

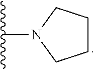 [SMILES: *N1CCCC1]

In some embodiments, at least one R^{Tb} is piperidinyl.

In some embodiments, at least one R^{Tb} is

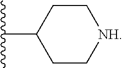 [SMILES: *C1CCNCC1]

In some embodiments, at least one R^{Tb} is

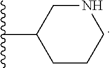 [SMILES: *C1CCCNC1]

In some embodiments, at least one R^{Tb} is

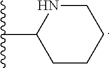 [SMILES: *C1CCCCN1]

In some embodiments, at least one R^{Tb} is

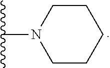 [SMILES: *N1CCCCC1]

In some embodiments, at least one R^{Tb} is 4- to 10-membered fused bicyclic heterocyclyl containing one N.

In some embodiments, at least one R^{Tb} is 4- to 10-membered fused bicyclic heterocyclyl containing one N, wherein the 4- to 10-membered fused bicyclic heterocyclyl is substituted with one or more R^{Tc}.

In some embodiments, at least one R^{Tb} is

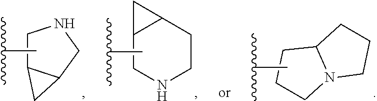

In some embodiments, at least one R^{Tb} is

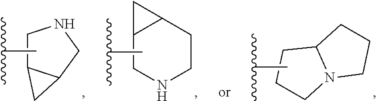

substituted with one or more R^{Tc}.

In some embodiments, at least one R^{Tb} is

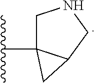 [SMILES: *C12CNCC1C2]

In some embodiments, at least one R^{Tb} is

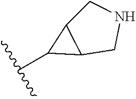 [SMILES: *C1C2CNCC12]

In some embodiments, at least one R^{Tb} is

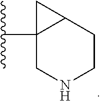 [SMILES: *C12CNCCC1C2]

In some embodiments, at least one R^{Tb} is

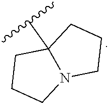 [SMILES: *C12CCCN1CCC2]

In some embodiments, at least one R^{Tb} is 5- to 10-membered bridged bicyclic heterocyclyl containing one N.

In some embodiments, at least one R^{Tb} is 5- to 10-membered bridged bicyclic heterocyclyl containing one N, wherein the 5- to 10-membered bridged bicyclic heterocyclyl is substituted with one or more R^{Tc}.

In some embodiments, at least one R^{Tb} is

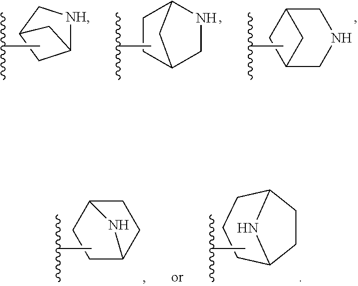

In some embodiments, at least one R^{Tb} is

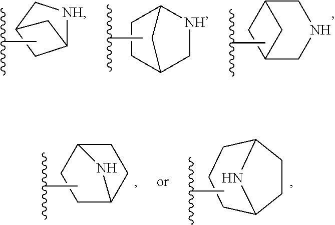

or substituted with one or more R^{Tc}.

In some embodiments, at least one R^{Tb} is

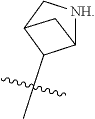 [SMILES: *C1C2CNC1C2]

In some embodiments, at least one R^{Tb} is

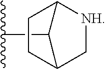 [SMILES: *C1C2CCC1NC2]

In some embodiments, at least one R^{Tb} is

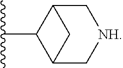 [SMILES: *C1C2CNCC1C2]

In some embodiments, at least one R^{Tb} is

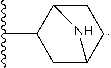 [SMILES: *C1CC2CCC1N2]

In some embodiments, at least one R^{Tb} is

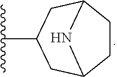 [SMILES: *C1CC2CCC(C1)N2]

In some embodiments, at least one R^{Tb} is

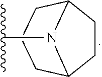 [SMILES: *N1C2CCCC1CC2]

In some embodiments, at least one R^{Tb} is 5- to 10-membered spiro bicyclic heterocyclyl containing one N.

In some embodiments, at least one R^{Tb} is 5- to 10-membered spiro bicyclic heterocyclyl containing one N, wherein the 5- to 10-membered spiro bicyclic heterocyclyl is substituted with one or more R^{Tc}.

In some embodiments, at least one R^{Tb} is

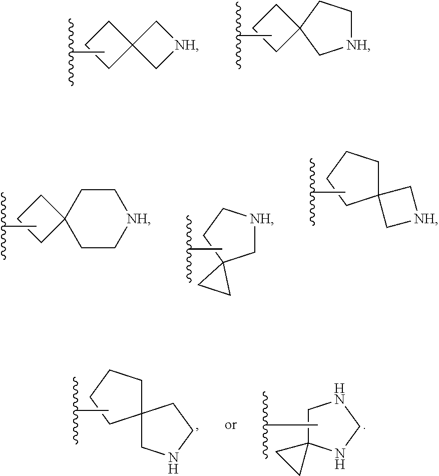

In some embodiments, at least one R^{Tb} is

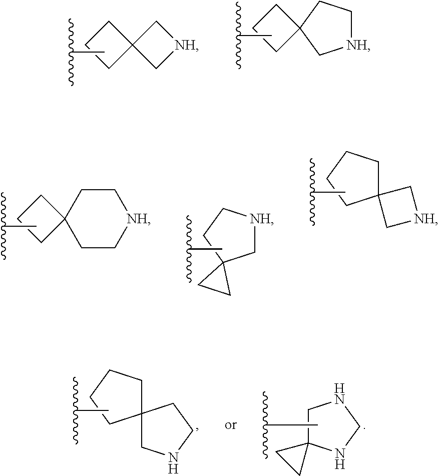

substituted with one or more R^{Tc}.

In some embodiments, at least one R^{Tb} is

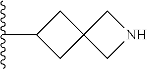 [SMILES: *C1CC2(CNC2)C1]

In some embodiments, at least one R^{Tb} is

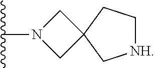 [SMILES: *N1CC2(CCNC2)C1]

In some embodiments, at least one R^{Tb} is

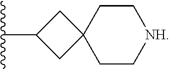 [SMILES: *C1CC2(CCNCC2)C1]

In some embodiments, at least one R^{Tb} is

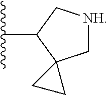 [SMILES: *C1CNCC12CC2]

In some embodiments, at least one R^{Tb} is

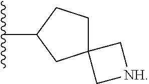 [SMILES: *C1CCC2(CNC2)C1]

In some embodiments, at least one R^{Tb} is

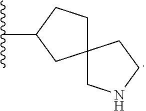 [SMILES: *C1CCC2(CCNC2)C1]

In some embodiments, at least one R^{Tb} is 4- to 10-membered monocyclic heterocyclyl containing one O (e.g., oxetanyl, tetrahydrofuranyl, or tetrahydropyranyl).

In some embodiments, at least one R^{Tb} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Tc}.

In some embodiments, at least one R^{Tb} is oxetanyl.

In some embodiments, at least one R^{Tb} is

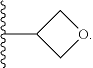 [SMILES: *C1COC1]

In some embodiments, at least one R^{Tb} is

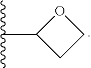 [SMILES: *C1CCO1]

In some embodiments, at least one R^{Tb} is tetrahydrofuranyl.

In some embodiments, at least one R^{Tb} is

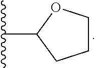 [SMILES: *C1CCCO1]

In some embodiments, at least one R^{Tb} is

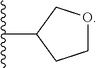 [SMILES: *C1CCOC1]

In some embodiments, at least one R^{Tb} is tetrahydropyranyl.

In some embodiments, at least one R^{Tb} is

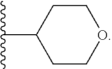 [SMILES: *C1CCOCC1]

In some embodiments, at least one R^{Tb} is

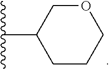 [SMILES: *C1CCCOC1]

In some embodiments, at least one R^{Tb} is

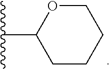 [SMILES: *C1CCCCO1]

In some embodiments, at least one R^{Tb} is 5- to 10-membered bridged bicyclic heterocyclyl containing one O.

In some embodiments, at least one R^{Tb} is 5- to 10-membered bridged bicyclic heterocyclyl containing one O, wherein the 5- to 10-membered bridged bicyclic heterocyclyl is substituted with one or more R^{Tc}.

In some embodiments, at least one R^{Tb} is 4- to 10-membered fused bicyclic heterocyclyl containing one O.

In some embodiments, at least one R^{Tb} is 4- to 10-membered fused bicyclic heterocyclyl containing one O, wherein the 4- to 10-membered fused heterocyclyl is substituted with one or more R^{Tc}.

In some embodiments, at least one R^{Tb} is 5- to 10-membered spiro bicyclic heterocyclyl containing one O.

In some embodiments, at least one R^{Tb} is 5- to 10-membered spiro bicyclic heterocyclyl containing one O, wherein the 5- to 10-membered spiro bicyclic heterocyclyl is substituted with one or more R^{Tc}.

In some embodiments, at least one R^{Tb} is

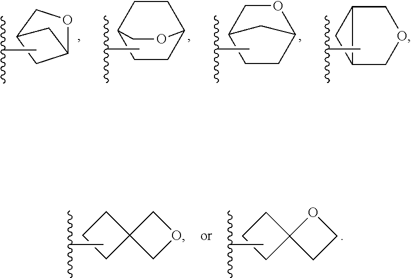

In some embodiments, at least one R^{Tb} is

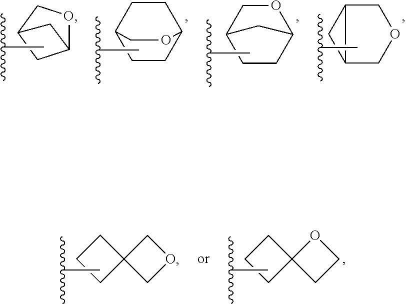

or substituted with one or more R^{Tc}.

In some embodiments, at least one R^{Tb} is

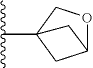 [SMILES: *C12COC(C1)C2]

In some embodiments, at least one R^{Tb} is

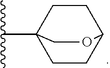 [SMILES: *C12CCC(CC1)OC2]

In some embodiments, at least one R^{Tb} is

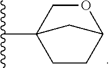 [SMILES: *C12CCC(C1)OC2]

In some embodiments, at least one R^{Tb} is

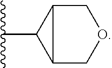 [SMILES: *C1C2COCC12]

In some embodiments, at least one R^{Tb} is

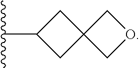 [SMILES: *C1CC2(COC2)C1]

In some embodiments, at least one R^{Tb} is

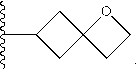 [SMILES: *C1CC2(CCO2)C1]

In some embodiments, at least one R^{Tb} is 4- to 10-membered monocyclic heterocyclyl containing one S (e.g., tetrahydrothiophenyl).

In some embodiments, at least one R^{Tb} is 4- to 10-membered monocyclic heterocyclyl containing one S (e.g., tetrahydrothiophenyl), wherein the 4- to 10-membered monocyclic heterocyclyl is substituted with one or more R^{Tc}.

In some embodiments, at least one R^{Tb} is tetrahydrothiophenyl.

In some embodiments, at least one R^{Tb} is

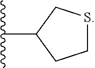 [SMILES: *C1CCSC1]

In some embodiments, at least one R^{Tb} is

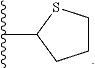 [SMILES: *C1CCCS1]

In some embodiments, at least one R^{Tb} is 4- to 10-membered heterocyclyl (e.g., monocyclic, fused bicyclic, bridged bicyclic, or spiro bicyclic) containing two heteroatoms selected from N, O, and S.

In some embodiments, at least one R^{Tb} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Tc}.

In some embodiments, at least one R^{Tb} is 4- to 10-membered monocyclic heterocyclyl containing two heteroatoms selected from N, O, and S.

In some embodiments, at least one R^{Tb} is 4- to 10-membered monocyclic heterocyclyl containing two heteroatoms selected from N, O, and S, wherein the 4- to 10-membered monocyclic heterocyclyl is substituted with one or more R^{Tc}.

In some embodiments, at least one R^{Tb} is 4- to 10-membered fused bicyclic heterocyclyl containing two heteroatoms selected from N, O, and S.

In some embodiments, at least one R^{Tb} is 4- to 10-membered fused bicyclic heterocyclyl containing two heteroatoms selected from N, O, and S, wherein the 4- to 10-membered fused bicyclic heterocyclyl is substituted with one or more R^{Tc}.

In some embodiments, at least one R^{Tb} is 5- to 10-membered bridged bicyclic heterocyclyl containing two heteroatoms selected from N, O, and S.

In some embodiments, at least one R^{Tb} is 5- to 10-membered bridged bicyclic heterocyclyl containing two heteroatoms selected from N, O, and S, wherein the 5- to 10-membered bridged bicyclic heterocyclyl is substituted with one or more R^{Tc}.

In some embodiments, at least one R^{Tb} is 5- to 10-membered spiro bicyclic heterocyclyl containing two heteroatoms selected from N, O, and S.

In some embodiments, at least one R^{Tb} is 5- to 10-membered spiro bicyclic heterocyclyl containing two heteroatoms selected from N, O, and S, wherein the 5- to 10-membered spiro bicyclic heterocyclyl is substituted with one or more R^{Tc}.

In some embodiments, at least one R^{Tb} is

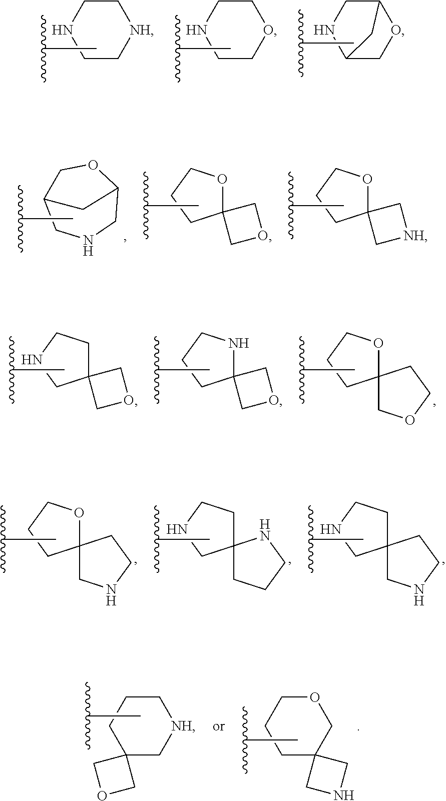

In some embodiments, at least one R^{Tb} is

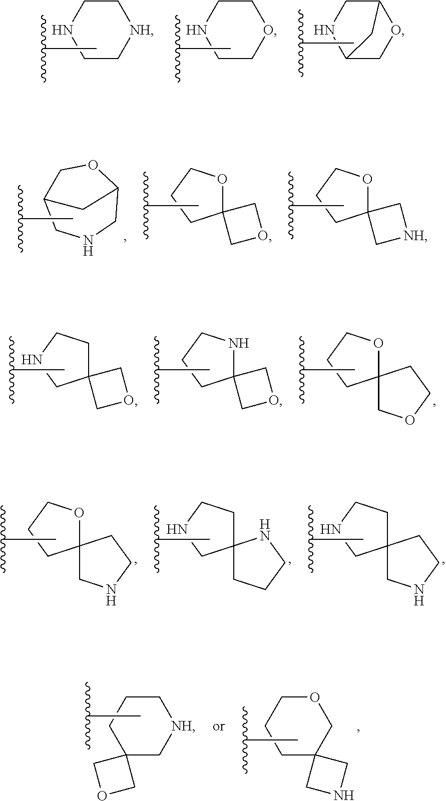

substituted with one or more R^{Tc}.

In some embodiments, at least one R^{Tb} is

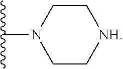 [SMILES: *N1CCNCC1]

In some embodiments, at least one R^{Tb} is

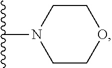 [SMILES: *N1CCOCC1]

In some embodiments, at least one R^{Tb} is

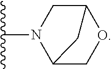 [SMILES: *N1CC2CC1CO2]

In some embodiments, at least one R^{Tb} is

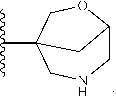 [SMILES: *C12CNCC(C1)OC2]

In some embodiments, at least one R^{Tb} is

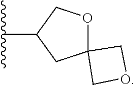 [SMILES: *C1COC2(COC2)C1]

In some embodiments, at least one R^{Tb} is

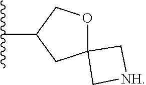 [SMILES: *C1COC2(CNC2)C1]

In some embodiments, at least one R^{Tb} is

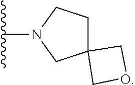 [SMILES: *N1CCC2(COC2)C1]

In some embodiments, at least one R^{Tb} is

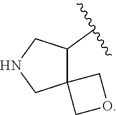 [SMILES: *C1CNCC12COC2]

In some embodiments, at least one R^{Tb} is

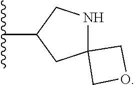 [SMILES: *C1CNC2(COC2)C1]

In some embodiments, at least one R^{Tb} is

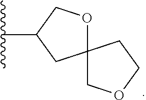 [SMILES: *C1COC2(CCOC2)C1]

In some embodiments, at least one R^{Tb} is

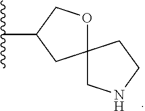 [SMILES: *C1COC2(CCNC2)C1]

In some embodiments, at least one R^{Tb} is

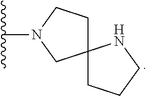 [SMILES: *N1CCC2(CCCN2)C1]

In some embodiments, at least one R^{Tb} is

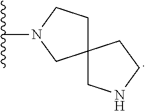 [SMILES: *N1CCC2(CCNC2)C1]

In some embodiments, at least one R^{Tb} is

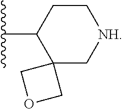 [SMILES: *C1CCNCC12COC2]

In some embodiments, at least one R^{Tb} is

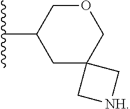 [SMILES: *C1COCC2(CNC2)C1]

In some embodiments, at least one R^{Tb} is C₆-C₁₀ aryl (e.g., phenyl).

In some embodiments, at least one R^{Tb} is C₆-C₁₀ aryl (e.g., phenyl) substituted with one or more R^{Tc}.

In some embodiments, at least one R^{Tb} is phenyl.

In some embodiments, at least one R^{Tb} is 5- to 10-membered heteroaryl.

In some embodiments, at least one R^{Tb} is 5- to 10-membered heteroaryl substituted with one or more R^{Tc}.

In some embodiments, at least one R^{Tb} is 5- to 10-membered heteroaryl containing at least one heteroatom selected from N, O, and S.

In some embodiments, at least one R^{Tb} is 5- to 10-membered heteroaryl containing at least one heteroatom selected from N, O, and S, wherein the heteroaryl is substituted with one or more R^{Tc}.

In some embodiments, at least one R^{Tb} is 5-membered heteroaryl containing at least one heteroatom selected from N, O, and S.

In some embodiments, at least one R^{Tb} is 5-membered heteroaryl containing at least one heteroatom selected from N, O, and S, wherein the heteroaryl is substituted with one or more R^{Tc}.

In some embodiments, at least one R^{Tb} is 5-membered heteroaryl containing one heteroatom selected from N, O, and S (e.g., pyrrolyl, furanyl, or thiophenyl).

In some embodiments, at least one R^{Tb} is 5-membered heteroaryl containing one heteroatom selected from N, O, and S (e.g., pyrrolyl, furanyl, or thiophenyl), wherein the heteroaryl is substituted with one or more R^{Tc}.

In some embodiments, at least one R^{Tb} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{Tb} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Tc}.

In some embodiments, at least one R^{Tb} is oxazolyl.

In some embodiments, at least one R^{Tb} is

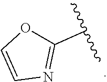 [SMILES: *c1ncco1]

In some embodiments, at least one R^{Tb} is thiazolyl.

In some embodiments, at least one R^{Tb} is

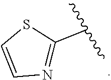 [SMILES: *c1nccs1]

In some embodiments, at least one R^{Tb} is pyrazolyl.

In some embodiments, at least one R^{Tb} is

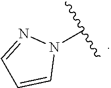 [SMILES: *n1cccn1]

In some embodiments, at least one R^{Tb} is

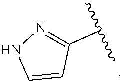 [SMILES: *c1cc[nH]n1]

In some embodiments, at least one R^{Tb} is isoxazolyl.

In some embodiments, at least one R^{Tb} is isothiazolyl.

In some embodiments, at least one R^{Tb} is imidazolyl.

In some embodiments, at least one R^{Tb} is 5-membered heteroaryl containing three heteroatoms selected from N, O, and S (e.g., triazolyl, oxadiazolyl, thiadiazolyl, or oxathiadiazolyl).

In some embodiments, at least one R^{Tb} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Tc}.

In some embodiments, at least one R^{Tb} is triazolyl.

In some embodiments, at least one R^{Tb} is

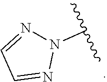 [SMILES: *n1nccn1]

In some embodiments, at least one R^{Tb} is

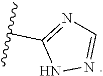 [SMILES: *c1ncn[nH]1]

In some embodiments, at least one R^{Tb} is

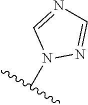 [SMILES: *n1cncn1]

In some embodiments, at least one R^{Tb} is oxadiazolyl.

In some embodiments, at least one R^{Tb} is

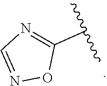 [SMILES: *c1ncno1]

In some embodiments, at least one R^{Tb} is thiadiazolyl.

In some embodiments, at least one R^{Tb} is

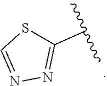 [SMILES: *c1nncs1]

In some embodiments, at least one R^{Tb} is

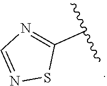 [SMILES: *c1ncns1]

In some embodiments, at least one R^{Tb} is 5-membered heteroaryl containing four heteroatoms selected from N, O, and S (e.g., tetrazolyl or oxatriazolyl).

In some embodiments, at least one R^{Tb} is 5-membered heteroaryl containing four heteroatoms selected from N, O, and S (e.g., tetrazolyl or oxatriazolyl), wherein the heteroaryl is substituted with one or more R^{Tc}.

In some embodiments, at least one R^{Tb} is tetrazolyl.

In some embodiments, at least one R^{Tb} is

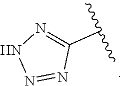 [SMILES: *c1nn[nH]n1]

In some embodiments, at least one R^{Tb} is oxatriazolyl.

In some embodiments, at least one R^{Tb} is 6-membered heteroaryl containing at least one heteroatom selected from N, O, and S.

In some embodiments, at least one R^{Tb} is 6-membered heteroaryl containing at least one heteroatom selected from N, O, and S, wherein the heteroaryl is substituted with one or more R^{Tc}.

In some embodiments, at least one R^{Tb} is 6-membered heteroaryl containing one heteroatom selected from N, O, and S (e.g., pyridinyl, pyranyl, or thiopyranyl).

In some embodiments, at least one R^{Tb} is 6-membered heteroaryl containing one heteroatom selected from N, O, and S (e.g., pyridinyl, pyranyl, or thiopyranyl), wherein the heteroaryl is substituted with one or more R^{Tc}.

In some embodiments, at least one R^{Tb} is pyridinyl.

In some embodiments, at least one R^{Tb} is

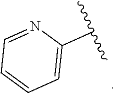 [SMILES: *c1ccccn1]

In some embodiments, at least one R^{Tb} is 6-membered heteroaryl containing two heteroatoms selected from N, O, and S (e.g., pyrimidinyl, pyridazinyl, or pyrazinyl).

In some embodiments, at least one R^{Tb} is 6-membered heteroaryl containing two heteroatoms selected from N, O, and S (e.g., pyrimidinyl, pyridazinyl, or pyrazinyl), wherein the heteroaryl is substituted with one or more R^{Tc}.

In some embodiments, at least one R^{Tb} is pyrimidinyl.

In some embodiments, at least one R^{Tb} is

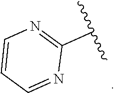 [SMILES: *c1ncccn1]

In some embodiments, at least one R^{Tb} is

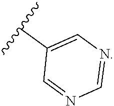 [SMILES: *c1cncnc1]

In some embodiments, at least one R^{Tb} is pyridazinyl.

In some embodiments, at least one R^{Tb} is

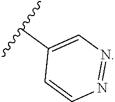 [SMILES: *c1ccnnc1]

In some embodiments, at least one R^{Tb} is pyrazinyl.

In some embodiments, at least one R^{Tb} is 9-membered heteroaryl containing at least one heteroatom selected from N, O, and S.

In some embodiments, at least one R^{Tb} is 9-membered heteroaryl containing at least one heteroatom selected from N, O, and S, wherein the heteroaryl is substituted with one or more R^{Tc}.

In some embodiments, at least one R^{Tb} is

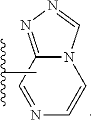

In some embodiments, at least one R^{Tb} is

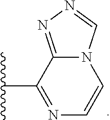 [SMILES: *c1nccn2cnnc12]

### Variable R^{Tb1}

In some embodiments, at least one R^{Tb1} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments, at least one R^{Tb1} is oxo.

In some embodiments, at least one R^{Tb1} is halogen (e.g., —F, —Cl, —Br, —I).

In some embodiments, at least one R^{Tb1} is cyano.

In some embodiments, at least one R^{Tb1} is —OH.

In some embodiments, at least one R^{Tb1} is —O(C₁-C₆ alkyl) (e.g., —O(methyl), —O(ethyl), —O(n-propyl), —O(isopropyl), —O(n-butyl), —O(t-butyl), —O(isobutyl), or —O(sec-butyl)).

In some embodiments, at least one R^{Tb1} is —NH₂.

In some embodiments, at least one R^{Tb1} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{Tb1} is —N(C₁-C₆ alkyl)₂ (e.g., e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, at least one R^{Tb1} is —C(═O)—H.

In some embodiments, at least one R^{Tb1} is —C(═O)—OH.

In some embodiments, at least one R^{Tb1} is —C(═O)—O(C₁-C₆ alkyl) (e.g., is —C(═O)—OCH₃, —C(═O)—OCH₂CH₃, —C(═O)—O(CH₂)₂CH₃, —C(═O)—O(CH₂)₃CH₃, —C(═O)—O(CH₂)₄CH₃, or —C(═O)—O(CH₂)₅CH₃).

In some embodiments, at least one R^{Tb1} is —C(═O)—NH₂.

In some embodiments, at least one R^{Tb1} is —C(═O)—NH(C₁-C₆ alkyl) ((e.g., —C(═O)—NHCH₃, —C(═O)—NHCH₂CH₃, —C(═O)—NH(CH₂)₂CH₃, —C(═O)—NH(CH₂)₃CH₃, —C(═O)—NH(CH₂)₄CH₃, or —C(═O)—NH(CH₂)₅CH₃)).

In some embodiments, at least one R^{Tb1} is —C(═O)—N(C₁-C₆ alkyl)₂ (e.g., —C(═O)—N(methyl)₂, —C(═O)—N(ethyl)₂, —C(═O)—N(n-propyl)₂, —C(═O)—N(isopropyl)₂, —C(═O)—N(n-butyl)₂, —C(═O)—N(t-butyl)₂, —C(═O)—N(isobutyl)₂, or —C(═O)—N(sec-butyl)₂).

In some embodiments, at least one R^{Tb1} is C₁-C₆ alkyl (e.g., methyl, ethyl, n-propyl, isopropyl, n-butyl, t-butyl, isobutyl, or sec-butyl).

In some embodiments, at least one R^{Tb1} is methyl.

In some embodiments, at least one R^{Tb1} is ethyl.

In some embodiments, at least one R^{Tb1} is n-propyl.

In some embodiments, at least one R^{Tb1} is isopropyl.

In some embodiments, at least one R^{Tb1} is n-butyl.

In some embodiments, at least one R^{Tb1} is C₂-C₆ alkenyl.

In some embodiments, at least one R^{Tb1} is C₂-C₆ alkynyl.

### Variable R^{Tc}

In some embodiments, at least one R^{Tc} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

In some embodiments, at least one R^{Tc} is oxo.

In some embodiments, at least one R^{Tc} is halogen (e.g., —F, —Cl, —Br, —I).

In some embodiments, at least one R^{Tc} is cyano.

In some embodiments, at least one R^{Tc} is —OH.

In some embodiments, at least one R^{Tc} is —O(C₁-C₆ alkyl) (e.g., —O(methyl), —O(ethyl), —O(n-propyl), —O(isopropyl), —O(n-butyl), —O(t-butyl), —O(isobutyl), or —O(sec-butyl)).

In some embodiments, at least one R^{Tc} is —NH₂.

In some embodiments, at least one R^{Tc} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{Tc} is —N(C₁-C₆ alkyl)₂ (e.g., e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, at least one R^{Tc} is —C(═O)—H.

In some embodiments, at least one R^{Tc} is —C(═O)—OH.

In some embodiments, at least one R^{Tc} is —C(═O)—O(C₁-C₆ alkyl) (e.g., is —C(═O)—OCH₃, —C(═O)—OCH₂CH₃, —C(═O)—O(CH₂)₂CH₃, —C(═O)—O(CH₂)₃CH₃, —C(═O)—O(CH₂)₄CH₃, or —C(═O)—O(CH₂)₅CH₃).

In some embodiments, at least one R^{Tc} is —C(═O)—NH₂.

In some embodiments, at least one R^{Tc} is —C(═O)—NH(C₁-C₆ alkyl) ((e.g., —C(═O)—NHCH₃, —C(═O)—NHCH₂CH₃, —C(═O)—NH(CH₂)₂CH₃, —C(═O)—NH(CH₂)₃CH₃, —C(═O)—NH(CH₂)₄CH₃, or —C(═O)—NH(CH₂)₅CH₃)).

In some embodiments, at least one R^{Tc} is —C(═O)—N(C₁-C₆ alkyl)₂ (e.g., —C(═O)—N(methyl)₂, —C(═O)—N(ethyl)₂, —C(═O)—N(n-propyl)₂, —C(═O)—N(isopropyl)₂, —C(═O)—N(n-butyl)₂, —C(═O)—N(t-butyl)₂, —C(═O)—N(isobutyl)₂, or —C(═O)—N(sec-butyl)₂).

In some embodiments, at least one R^{Tc} is C₁-C₆ alkyl (e.g., methyl, ethyl, n-propyl, isopropyl, n-butyl, t-butyl, isobutyl, or sec-butyl).

In some embodiments, at least one R^{Tc} is methyl.

In some embodiments, at least one R^{Tc} is ethyl.

In some embodiments, at least one R^{Tc} is n-propyl.

In some embodiments, at least one R^{Tc} is isopropyl.

In some embodiments, at least one R^{Tc} is n-butyl.

In some embodiments, at least one R^{Tc} is C₂-C₆ alkenyl.

In some embodiments, at least one R^{Tc} is C₂-C₆ alkynyl.

### Variable R^{b}

In some embodiments, at least one R^{b} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc}.

In some embodiments, at least one R^{b} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{b} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{Tc}.

In some embodiments, at least one R^{b} is C₁-C₆ alkyl (e.g., methyl, ethyl, n-propyl, isopropyl, n-butyl, t-butyl, isobutyl, or sec-butyl).

In some embodiments, at least one R^{b} is C₁-C₆ alkyl (e.g., methyl, ethyl, n-propyl, isopropyl, n-butyl, t-butyl, isobutyl, or sec-butyl) substituted with one or more R^{Tc}.

In some embodiments, at least one R^{b} is methyl.

In some embodiments, at least one R^{b} is ethyl.

In some embodiments, at least one R^{b} is n-propyl.

In some embodiments, at least one R^{b} is isopropyl.

In some embodiments, at least one R^{b} is n-butyl.

In some embodiments, at least one R^{b} is C₂-C₆ alkenyl.

In some embodiments, at least one R^{b} is C₂-C₆ alkenyl substituted with one or more R^{Tc}.

In some embodiments, at least one R^{b} is C₂-C₆ alkynyl.

In some embodiments, at least one R^{b} is C₂-C₆ alkynyl substituted with one or more R^{Tc}.

In some embodiments, at least one R^{b} is C₃-C₆ cycloalkyl.

In some embodiments, at least one R^{b} is C₃-C₆ cycloalkyl substituted with one or more R^{Tc}.

In some embodiments, at least one R^{b} is C₃-C₆ monocyclic cycloalkyl.

In some embodiments, at least one R^{b} is C₃-C₆ monocyclic cycloalkyl substituted with one or more R^{Tc}.

In some embodiments, at least one R^{b} is cyclopropyl, cyclobutyl, cyclopentyl, cyclohexyl, cycloheptyl, or cyclooctyl.

In some embodiments, at least one R^{b} is C₃-C₆ bicyclic cycloalkyl.

In some embodiments, at least one R^{b} is C₃-C₆ bicyclic cycloalkyl substituted with one or more R^{Tc}.

In some embodiments, at least one R^{b} is bicyclocyclobutyl, bicyclocyclopentyl, or bicyclocyclohexyl.

In some embodiments, at least one R^{b} is C₄-C₆ fused bicyclic cycloalkyl.

In some embodiments, at least one R^{b} is C₄-C₆ fused bicyclic cycloalkyl substituted with one or more R^{Tc}.

In some embodiments, at least one R^{b} is

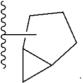

In some embodiments, at least one R^{b} is

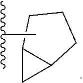

substituted with one or more R^{Tc}.

In some embodiments, at least one R^{b} is

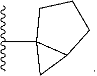 [SMILES: *C12CCCC1C2]

In some embodiments, at least one R^{b} is

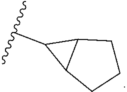 [SMILES: *C1C2CCCC12]

In some embodiments, at least one R^{b} is C₅-C₆ bridged bicyclic cycloalkyl.

In some embodiments, at least one R^{b} is C₅-C₆ bridged bicyclic cycloalkyl substituted with one or more R^{Tc}.

In some embodiments, at least one R^{b} is

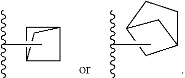

In some embodiments, at least one R^{b} is

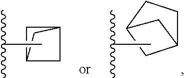

substituted with one or more R^{Tc}.

In some embodiments, at least one R^{b} is

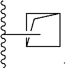

In some embodiments, at least one R^{b} is

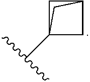 [SMILES: *C12CC(C1)C2]

In some embodiments, at least one R^{b} is

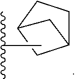

In some embodiments, at least one R^{b} is

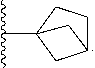 [SMILES: *C12CCC(C1)C2]

In some embodiments, at least one R^{b} is C₅-C₆ spiro bicyclic cycloalkyl.

In some embodiments, at least one R^{b} is C₅-C₆ spiro bicyclic cycloalkyl substituted with one or more R^{Tc}.

In some embodiments, at least one R^{b} is spiro[2.2]pentyl or spiro[2.3]hexyl.

In some embodiments, at least one R^{b} is spiro[2.2]pentyl or spiro[2.3]hexyl, substituted with one or more R^{Tc}.

In some embodiments, at least one R^{b} is

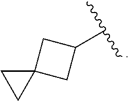 [SMILES: *C1CC2(CC2)C1]

In some embodiments, at least one R^{b} is

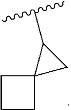 [SMILES: *C1CC12CCC2]

In some embodiments, at least one R^{b} is 4- to 10-membered heterocyclyl.

In some embodiments, at least one R^{b} is 4- to 10-membered heterocyclyl substituted with one or more R^{Tc}.

In some embodiments, at least one R^{b} is 4- to 10-membered monocyclic heterocyclyl.

In some embodiments, at least one R^{b} is 4- to 10-membered monocyclic heterocyclyl substituted with one or more R^{Tc}.

In some embodiments, at least one R^{b} is 4- to 10-membered bicyclic heterocyclyl.

In some embodiments, at least one R^{b} is 4- to 10-membered bicyclic heterocyclyl substituted with one or more R^{Tc}.

In some embodiments, at least one R^{b} is 4- to 10-membered fused bicyclic heterocyclyl.

In some embodiments, at least one R^{b} is 4- to 10-membered fused bicyclic heterocyclyl substituted with one or more R^{Tc}.

In some embodiments, at least one R^{b} is 5- to 10-membered bridged bicyclic heterocyclyl.

In some embodiments, at least one R^{b} is 5- to 10-membered bridged bicyclic heterocyclyl substituted with one or more R^{Tc}.

In some embodiments, at least one R^{b} is 5- to 10-membered spiro bicyclic heterocyclyl.

In some embodiments, at least one R^{b} is 5- to 10-membered spiro bicyclic heterocyclyl substituted with one or more R^{Tc}.

In some embodiments, at least one R^{b} is 4- to 10-membered heterocyclyl containing at least one heteroatom selected from N, O, and S.

In some embodiments, at least one R^{b} is 4- to 10-membered heterocyclyl containing at least one heteroatom selected from N, O, and S, wherein the 4- to 10-membered heterocyclyl is substituted with one or more R^{Tc}.

In some embodiments, at least one R^{b} is 4- to 10-membered monocyclic heterocyclyl containing at least one heteroatom selected from N, O, and S.

In some embodiments, at least one R^{b} is 4- to 10-membered monocyclic heterocyclyl containing at least one heteroatom selected from N, O, and S, wherein the 4- to 10-membered heterocyclyl is substituted with one or more R^{Tc}.

In some embodiments, at least one R^{b} is 4- to 10-membered monocyclic heterocyclyl containing one N (e.g., azetidinyl, pyrrolidinyl, or piperidinyl).

In some embodiments, at least one R^{b} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Tc}.

In some embodiments, at least one R^{b} is azetidinyl.

In some embodiments, at least one R^{b} is azetidinyl substituted with one or more R^{Tc}.

In some embodiments, at least one R^{b} is

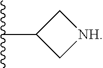 [SMILES: *C1CNC1]

In some embodiments, at least one R^{b} is

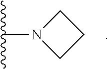 [SMILES: *N1CCC1]

In some embodiments, at least one R^{b} is

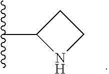 [SMILES: *C1CCN1]

In some embodiments, at least one R^{b} is pyrrolidinyl.

In some embodiments, at least one R^{b} is

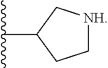 [SMILES: *C1CCNC1]

In some embodiments, at least one R^{b} is

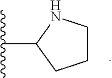 [SMILES: *C1CCCN1]

In some embodiments, at least one R^{b} is

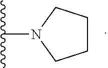 [SMILES: *N1CCCC1]

In some embodiments, at least one R^{b} is piperidinyl.

In some embodiments, at least one R^{b} is

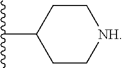 [SMILES: *C1CCNCC1]

In some embodiments, at least one R^{b} is

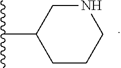 [SMILES: *C1CCCNC1]

In some embodiments, at least one R^{b} is

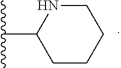 [SMILES: *C1CCCCN1]

In some embodiments, at least one R^{b} is

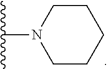 [SMILES: *N1CCCCC1]

In some embodiments, at least one R^{b} is 4- to 10-membered fused bicyclic heterocyclyl containing one N.

In some embodiments, at least one R^{b} is 4- to 10-membered fused bicyclic heterocyclyl containing one N, wherein the 4- to 10-membered fused bicyclic heterocyclyl is substituted with one or more R^{Tc}.

In some embodiments, at least one R^{b} is

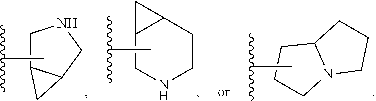

In some embodiments, at least one R^{b} is

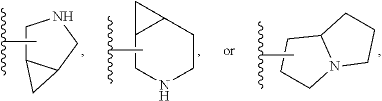

substituted with one or more R^{Tc}.

In some embodiments, at least one R^{b} is

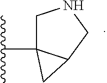 [SMILES: *C12CNCC1C2]

In some embodiments, at least one R^{b} is

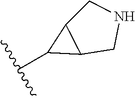 [SMILES: *C1C2CNCC12]

In some embodiments, at least one R^{b} is H

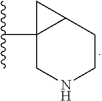 [SMILES: *C12CNCCC1C2]

In some embodiments, at least one R^{b} is

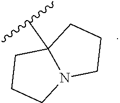 [SMILES: *C12CCCN1CCC2]

In some embodiments, at least one R^{b} is 5- to 10-membered bridged bicyclic heterocyclyl containing one N.

In some embodiments, at least one R^{b} is 5- to 10-membered bridged bicyclic heterocyclyl containing one N, wherein the 5- to 10-membered bridged bicyclic heterocyclyl is substituted with one or more R^{Tc}.

In some embodiments, at least one R^{b} is

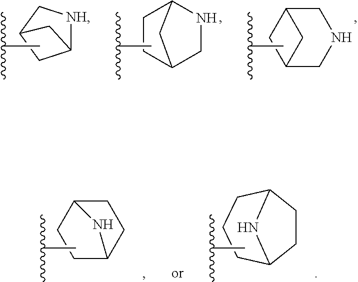

In some embodiments, at least one R^{b} is

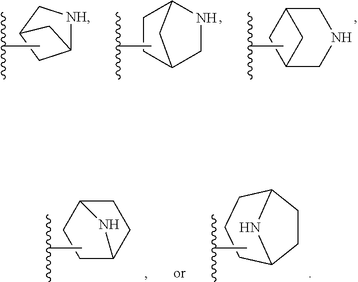

substituted with one or more R^{Tc}.

In some embodiments, at least one R^{b} is

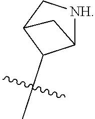 [SMILES: *C1C2CNC1C2]

In some embodiments, at least one R^{b} is

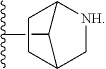 [SMILES: *C1C2CCC1NC2]

In some embodiments, at least one R is

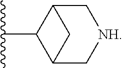 [SMILES: *C1C2CNCC1C2]

In some embodiments, at least one R^{b} is

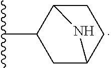 [SMILES: *C1CC2CCC1N2]

In some embodiments, at least one R^{b} is

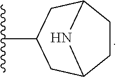 [SMILES: *C1CC2CCC(C1)N2]

In some embodiments, at least one R^{b} is

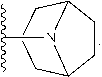 [SMILES: *N1C2CCCC1CC2]

In some embodiments, at least one R^{b} is 5- to 10-membered spiro bicyclic heterocyclyl containing one N.

In some embodiments, at least one R^{b} is 5- to 10-membered spiro bicyclic heterocyclyl containing one N, wherein the 5- to 10-membered spiro bicyclic heterocyclyl is substituted with one or more R^{Tc}.

In some embodiments, at least one R^{b} is

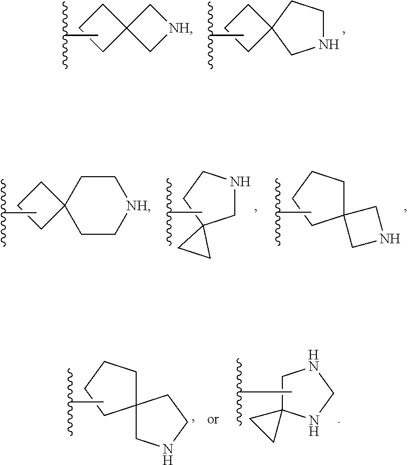

In some embodiments, at least one R^{b} is

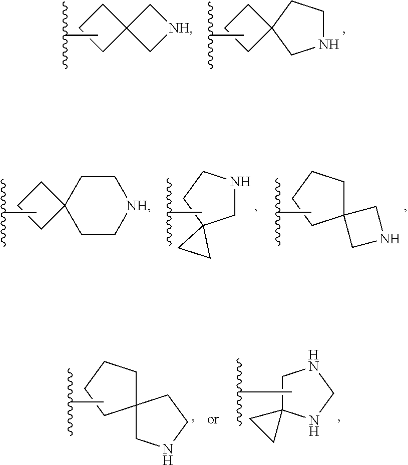

substituted with one or more R^{Tc}.

In some embodiments, at least one R^{b} is

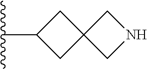 [SMILES: *C1CC2(CNC2)C1]

In some embodiments, at least one R^{b} is

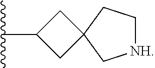 [SMILES: *C1CC2(CCNC2)C1]

In some embodiments, at least one R^{b} is

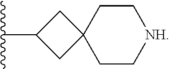 [SMILES: *C1CC2(CCNCC2)C1]

In some embodiments, at least one R^{b} is

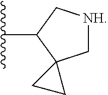 [SMILES: *C1CNCC12CC2]

In some embodiments, at least one R^{b} is

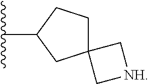 [SMILES: *C1CCC2(CNC2)C1]

In some embodiments, at least one R^{b} is

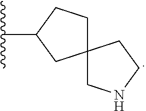 [SMILES: *C1CCC2(CCNC2)C1]

In some embodiments, at least one R^{b} is 4- to 10-membered monocyclic heterocyclyl containing one O (e.g., oxetanyl, tetrahydrofuranyl, or tetrahydropyranyl).

In some embodiments, at least one R^{b} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Tc}.

In some embodiments, at least one R^{b} is oxetanyl.

In some embodiments, at least one R^{b} is

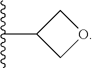 [SMILES: *C1COC1]

In some embodiments, at least one R^{b} is

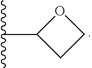 [SMILES: *C1CCO1]

In some embodiments, at least one R^{b} is tetrahydrofuranyl.

In some embodiments, at least one R^{b} is

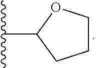 [SMILES: *C1CCCO1]

In some embodiments, at least one R^{b} is

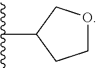 [SMILES: *C1CCOC1]

In some embodiments, at least one R^{b} is tetrahydropyranyl.

In some embodiments, at least one R^{b} is

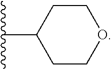 [SMILES: *C1CCOCC1]

In some embodiments, at least one R^{b} is

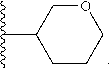 [SMILES: *C1CCCOC1]

In some embodiments, at least one R^{b} is

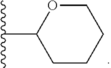 [SMILES: *C1CCCCO1]

In some embodiments, at least one R^{b} is 5- to 10-membered bridged bicyclic heterocyclyl containing one O.

In some embodiments, at least one R^{b} is 5- to 10-membered bridged bicyclic heterocyclyl containing one O, wherein the 5- to 10-membered bridged bicyclic heterocyclyl is substituted with one or more R^{Tc}.

In some embodiments, at least one R^{b} is 4- to 10-membered fused bicyclic heterocyclyl containing one O.

In some embodiments, at least one R^{b} is 4- to 10-membered fused bicyclic heterocyclyl containing one O, wherein the 4- to 10-membered fused heterocyclyl is substituted with one or more R^{Tc}.

In some embodiments, at least one R^{b} is 5- to 10-membered spiro bicyclic heterocyclyl containing one O.

In some embodiments, at least one R^{b} is 5- to 10-membered spiro bicyclic heterocyclyl containing one O, wherein the 5- to 10-membered spiro bicyclic heterocyclyl is substituted with one or more R^{Tc}.

In some embodiments, at least one R^{b} is

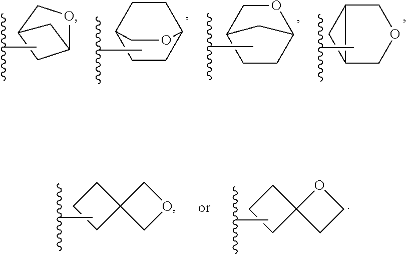

In some embodiments, at least one R^{b} is

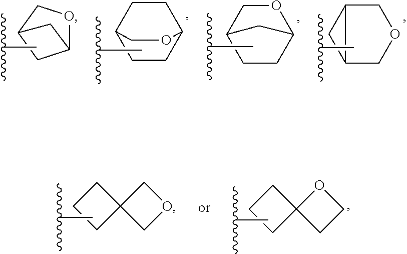

substituted with one or more R^{c}.

In some embodiments, at least one R^{b} is

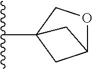 [SMILES: *C12COC(C1)C2]

In some embodiments, at least one R^{b} is

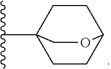 [SMILES: *C12CCC(CC1)OC2]

In some embodiments, at least one R^{b} is

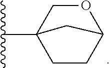 [SMILES: *C12CCC(C1)OC2]

In some embodiments, at least one R^{b} is

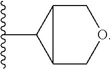 [SMILES: *C1C2COCC12]

In some embodiments, at least one R^{b} is

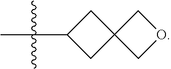 [SMILES: *C1CC2(COC2)C1]

In some embodiments, at least one R^{b} is

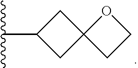 [SMILES: *C1CC2(CCO2)C1]

In some embodiments, at least one R^{b} is 4- to 10-membered monocyclic heterocyclyl containing one S (e.g., tetrahydrothiophenyl).

In some embodiments, at least one R^{b} is 4- to 10-membered monocyclic heterocyclyl containing one S (e.g., tetrahydrothiophenyl), wherein the 4- to 10-membered monocyclic heterocyclyl is substituted with one or more R^{Tc}.

In some embodiments, at least one R^{b} is tetrahydrothiophenyl.

In some embodiments, at least one R^{b} is

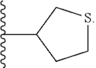 [SMILES: *C1CCSC1]

In some embodiments, at least one R^{b} is

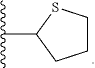 [SMILES: *C1CCCS1]

In some embodiments, at least one R^{b} is 4- to 10-membered heterocyclyl (e.g., monocyclic, fused bicyclic, bridged bicyclic, or spiro bicyclic) containing two heteroatoms selected from N, O, and S.

In some embodiments, at least one R^{b} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Tc}.

In some embodiments, at least one R^{b} is 4- to 10-membered monocyclic heterocyclyl containing two heteroatoms selected from N, O, and S.

In some embodiments, at least one R^{b} is 4- to 10-membered monocyclic heterocyclyl containing two heteroatoms selected from N, O, and S, wherein the 4- to 10-membered monocyclic heterocyclyl is substituted with one or more R^{Tc}.

In some embodiments, at least one R^{b} is 4- to 10-membered fused bicyclic heterocyclyl containing two heteroatoms selected from N, O, and S.

In some embodiments, at least one R^{b} is 4- to 10-membered fused bicyclic heterocyclyl containing two heteroatoms selected from N, O, and S, wherein the 4- to 10-membered fused bicyclic heterocyclyl is substituted with one or more R^{Tc}.

In some embodiments, at least one R^{b} is 5- to 10-membered bridged bicyclic heterocyclyl containing two heteroatoms selected from N, O, and S.

In some embodiments, at least one R^{b} is 5- to 10-membered bridged bicyclic heterocyclyl containing two heteroatoms selected from N, O, and S, wherein the 5- to 10-membered bridged bicyclic heterocyclyl is substituted with one or more R^{Tc}.

In some embodiments, at least one R^{b} is 5- to 10-membered spiro bicyclic heterocyclyl containing two heteroatoms selected from N, O, and S.

In some embodiments, at least one R^{b} is 5- to 10-membered spiro bicyclic heterocyclyl containing two heteroatoms selected from N, O, and S, wherein the 5- to 10-membered spiro bicyclic heterocyclyl is substituted with one or more R^{Tc}.

In some embodiments, at least one R^{b} is

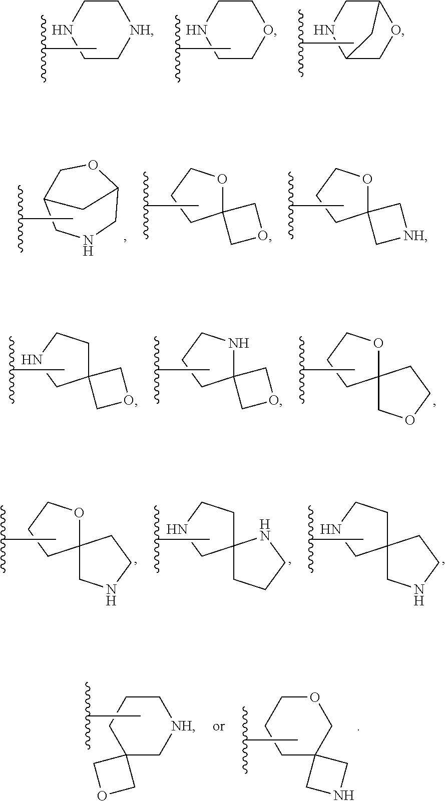

In some embodiments, at least one R^{b} is

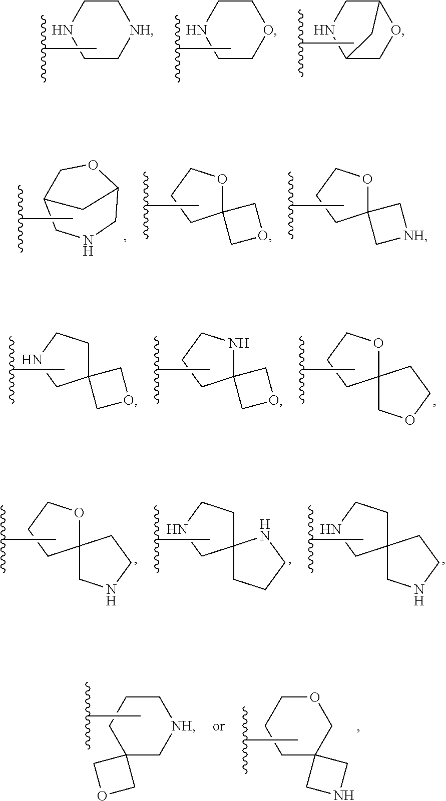

substituted with one or more R^{Tc}.

In some embodiments, at least one R^{b} is

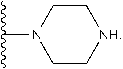 [SMILES: *N1CCNCC1]

In some embodiments, at least one R^{b} is

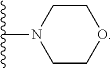 [SMILES: *N1CCOCC1]

In some embodiments, at least one R^{b} is

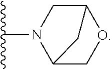 [SMILES: *N1CC2CC1CO2]

In some embodiments, at least one R^{b} is

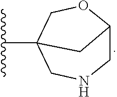 [SMILES: *C12CNCC(C1)OC2]

In some embodiments, at least one R^{b} is

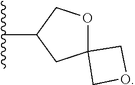 [SMILES: *C1COC2(COC2)C1]

In some embodiments, at least one R^{b} is

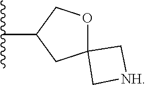 [SMILES: *C1COC2(CNC2)C1]

In some embodiments, at least one R^{b} is

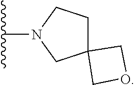 [SMILES: *N1CCC2(COC2)C1]

In some embodiments, at least one R^{b} is

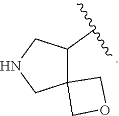 [SMILES: *C1CNCC12COC2]

In some embodiments, at least one R^{b} is

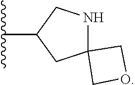 [SMILES: *C1CNC2(COC2)C1]

In some embodiments, at least one R^{b} is

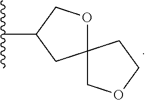 [SMILES: *C1COC2(CCOC2)C1]

In some embodiments, at least one R^{b} is H

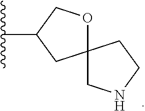 [SMILES: *C1COC2(CCNC2)C1]

In some embodiments, at least one R^{b} is

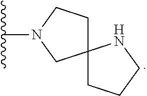 [SMILES: *N1CCC2(CCCN2)C1]

In some embodiments, at least one R^{b} is H

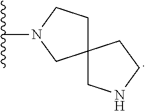 [SMILES: *N1CCC2(CCNC2)C1]

In some embodiments, at least one R^{b} is

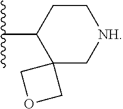 [SMILES: *C1CCNCC12COC2]

In some embodiments, at least one R^{b} is

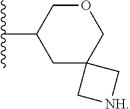 [SMILES: *C1COCC2(CNC2)C1]

In some embodiments, at least one R^{b} is C₆-C₁₀ aryl (e.g., phenyl).

In some embodiments, at least one R^{b} is C₆-C₁₀ aryl (e.g., phenyl) substituted with one or more R^{Tc}.

In some embodiments, at least one R^{b} is phenyl.

In some embodiments, at least one R^{b} is 5- to 10-membered heteroaryl.

In some embodiments, at least one R^{b} is 5- to 10-membered heteroaryl substituted with one or more R^{Tc}.

In some embodiments, at least one R^{b} is 5- to 10-membered heteroaryl containing at least one heteroatom selected from N, O, and S.

In some embodiments, at least one R^{b} is 5- to 10-membered heteroaryl containing at least one heteroatom selected from N, O, and S, wherein the heteroaryl is substituted with one or more R^{Tc}.

In some embodiments, at least one R^{b} is 5-membered heteroaryl containing at least one heteroatom selected from N, O, and S.

In some embodiments, at least one R^{b} is 5-membered heteroaryl containing at least one heteroatom selected from N, O, and S, wherein the heteroaryl is substituted with one or more R^{Tc}.

In some embodiments, at least one R^{b} is 5-membered heteroaryl containing one heteroatom selected from N, O, and S (e.g., pyrrolyl, furanyl, or thiophenyl).

In some embodiments, at least one R^{b} is 5-membered heteroaryl containing one heteroatom selected from N, O, and S (e.g., pyrrolyl, furanyl, or thiophenyl), wherein the heteroaryl is substituted with one or more R^{Tc}.

In some embodiments, at least one R^{b} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{b} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Tc}.

In some embodiments, at least one R^{b} is oxazolyl.

In some embodiments, at least one R^{b} is

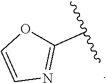 [SMILES: *c1ncco1]

In some embodiments, at least one R^{b} is thiazolyl.

In some embodiments, at least one R^{b} is

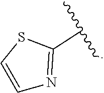 [SMILES: *c1nccs1]

In some embodiments, at least one R^{b} is pyrazolyl.

In some embodiments, at least one R^{b} is

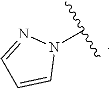 [SMILES: *n1cccn1]

In some embodiments, at least one R^{b} is

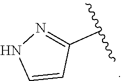 [SMILES: *c1cc[nH]n1]

In some embodiments, at least one R is isoxazolyl.

In some embodiments, at least one R^{b} is isothiazolyl.

In some embodiments, at least one R is imidazolyl.

In some embodiments, at least one R^{b} is 5-membered heteroaryl containing three heteroatoms selected from N, O, and S (e.g., triazolyl, oxadiazolyl, thiadiazolyl, or oxathiadiazolyl).

In some embodiments, at least one R^{b} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Tc}.

In some embodiments, at least one R^{b} is triazolyl.

In some embodiments, at least one R^{b} is

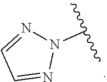 [SMILES: *n1nccn1]

In some embodiments, at least one R^{b} is

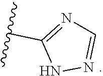 [SMILES: *c1ncn[nH]1]

In some embodiments, at least one R^{b} is

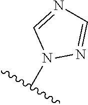 [SMILES: *n1cncn1]

In some embodiments, at least one R^{b} is oxadiazolyl.

In some embodiments, at least one R^{b} is

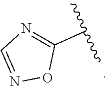 [SMILES: *c1ncno1]

In some embodiments, at least one R^{b} is thiadiazolyl.

In some embodiments, at least one R^{b} is

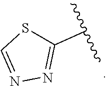 [SMILES: *c1nncs1]

In some embodiments, at least one R^{b} is

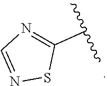 [SMILES: *c1ncns1]

In some embodiments, at least one R^{b} is 5-membered heteroaryl containing four heteroatoms selected from N, O, and S (e.g., tetrazolyl or oxatriazolyl).

In some embodiments, at least one R^{b} is 5-membered heteroaryl containing four heteroatoms selected from N, O, and S (e.g., tetrazolyl or oxatriazolyl), wherein the heteroaryl is substituted with one or more R^{Tc}.

In some embodiments, at least one R^{b} is tetrazolyl.

In some embodiments, at least one R^{b} is

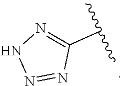 [SMILES: *c1nn[nH]n1]

In some embodiments, at least one R^{b} is oxatriazolyl.

In some embodiments, at least one R^{b} is 6-membered heteroaryl containing at least one heteroatom selected from N, O, and S.

In some embodiments, at least one R^{b} is 6-membered heteroaryl containing at least one heteroatom selected from N, O, and S, wherein the heteroaryl is substituted with one or more R^{Tc}.

In some embodiments, at least one R^{b} is 6-membered heteroaryl containing one heteroatom selected from N, O, and S (e.g., pyridinyl, pyranyl, or thiopyranyl).

In some embodiments, at least one R^{b} is 6-membered heteroaryl containing one heteroatom selected from N, O, and S (e.g., pyridinyl, pyranyl, or thiopyranyl), wherein the heteroaryl is substituted with one or more R^{Tc}.

In some embodiments, at least one R^{b} is pyridinyl.

In some embodiments, at least one R^{b} is

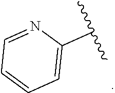 [SMILES: *c1ccccn1]

In some embodiments, at least one R^{b} is 6-membered heteroaryl containing two heteroatoms selected from N, O, and S (e.g., pyrimidinyl, pyridazinyl, or pyrazinyl).

In some embodiments, at least one R^{b} is 6-membered heteroaryl containing two heteroatoms selected from N, O, and S (e.g., pyrimidinyl, pyridazinyl, or pyrazinyl), wherein the heteroaryl is substituted with one or more R^{Tc}.

In some embodiments, at least one R^{b} is pyrimidinyl.

In some embodiments, at least one R^{b} is

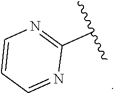 [SMILES: *c1ncccn1]

In some embodiments, at least one R^{b} is

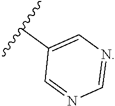 [SMILES: *c1cncnc1]

In some embodiments, at least one R^{b} is pyridazinyl.

In some embodiments, at least one R^{b} is

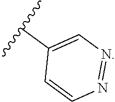 [SMILES: *c1ccnnc1]

In some embodiments, at least one R^{b} is pyrazinyl.

In some embodiments, at least one R^{b} is 9-membered heteroaryl containing at least one heteroatom selected from N, O, and S.

In some embodiments, at least one R^{b} is 9-membered heteroaryl containing at least one heteroatom selected from N, O, and S, wherein the heteroaryl is substituted with one or more R^{Tc}.

In some embodiments, at least one R^{b} is

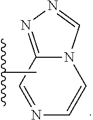

In some embodiments, at least one R^{b} is

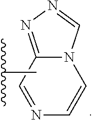

### Variable R^{c}

In some embodiments, at least one R^{c} is C₁-C₆ alkyl, C₂-C₆ alkenyl, or C₂-C₆ alkynyl.

In some embodiments, at least one R^{c} is C₁-C₆ alkyl (e.g., methyl, ethyl, n-propyl, isopropyl, n-butyl, t-butyl, isobutyl, or sec-butyl).

In some embodiments, at least one R^{c} is methyl.

In some embodiments, at least one R^{c} is ethyl.

In some embodiments, at least one R^{c} is C₂-C₆ alkenyl.

In some embodiments, at least one R^{c}C₂-C₆ alkynyl.

### Variable Ring A

In some embodiments, ring A is

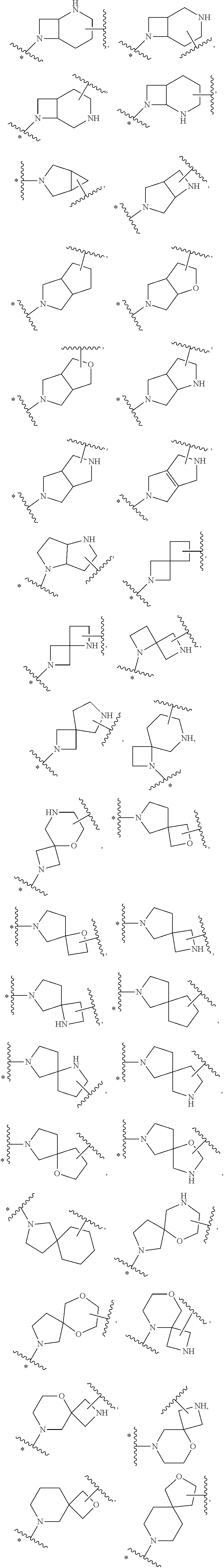

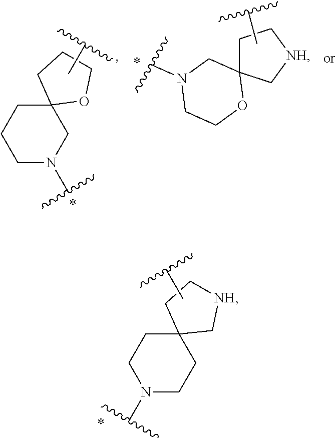

where * indicates connectivity to the carbonyl group at triazolopyridine (i.e., not the connectivity to Z)

In some embodiments, ring A is

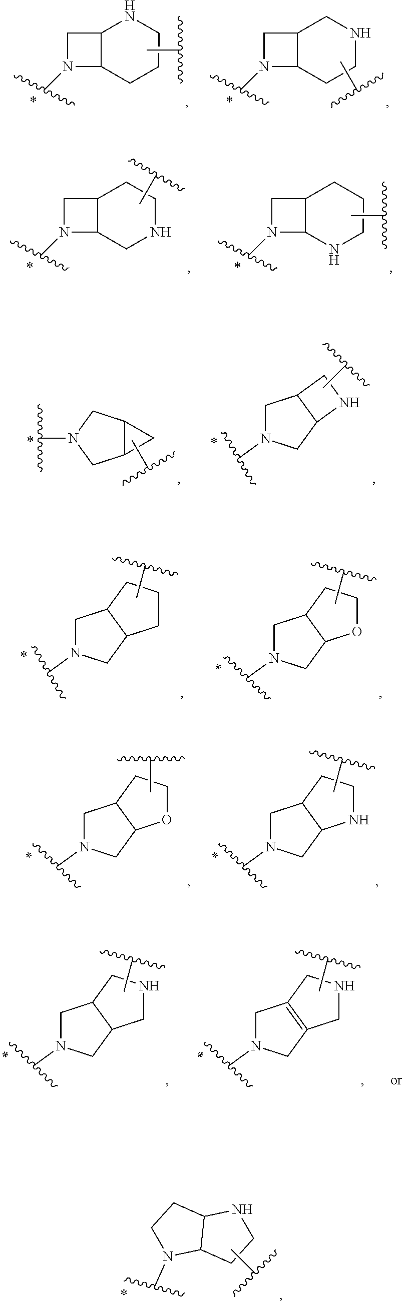

where * indicates connectivity to the carbonyl group at triazolopyridine.

In some embodiments, ring A is

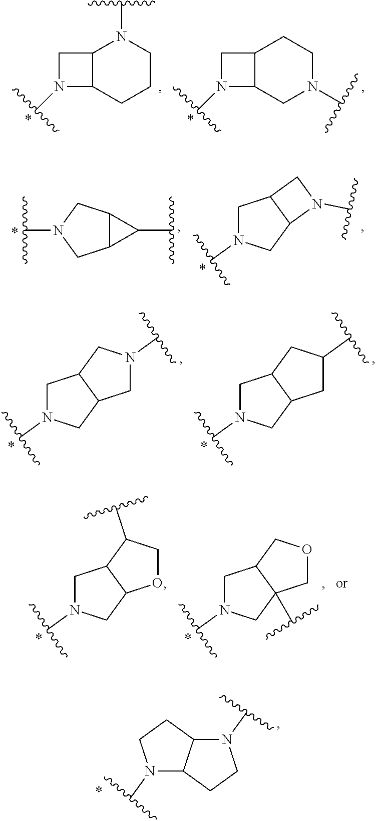

where * indicates connectivity to the carbonyl group at triazolopyridine.

In some embodiments, ring A is

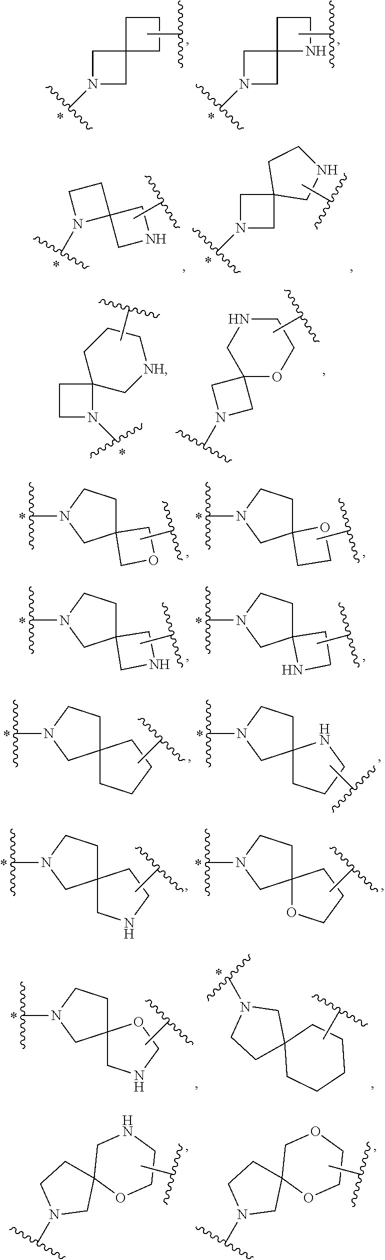

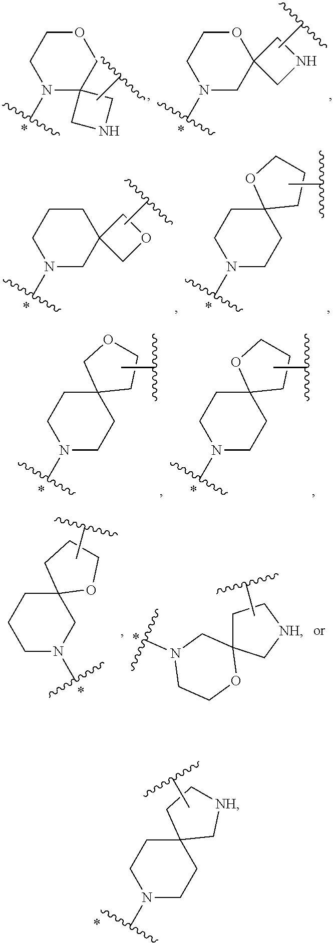

where * indicates connectivity to the carbonyl group at triazolopyridine.

In some embodiments, ring A is

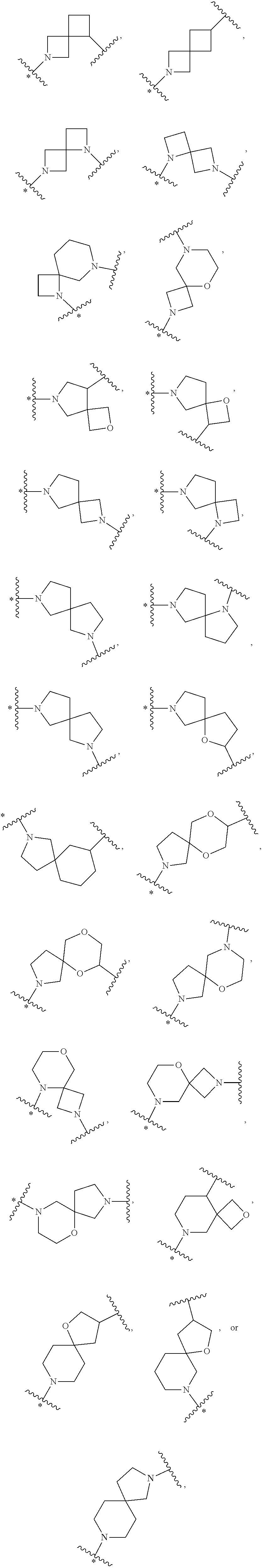

where * indicates connectivity to the carbonyl group at tri azolopyridine.

In some embodiments, ring A is

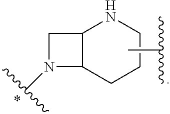

In some embodiments, ring A is

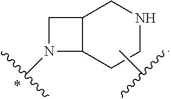

In some embodiments, ring A is

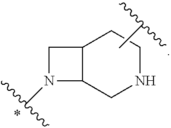

In some embodiments, ring A is

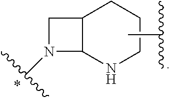

In some embodiments, ring A is

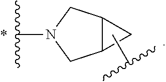

In some embodiments, ring A is H

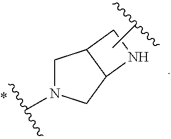

In some embodiments, ring A is

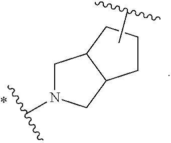

In some embodiments, ring A is

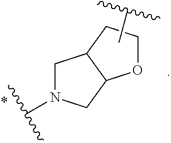

In some embodiments, ring A is

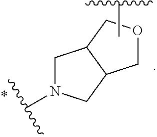

In some embodiments, ring A is

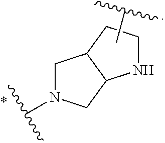

In some embodiments, ring A is

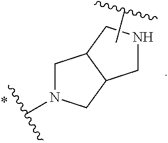

In some embodiments, ring A is

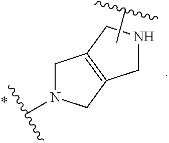

In some embodiments, ring A is

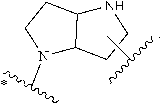

In some embodiments, ring A is

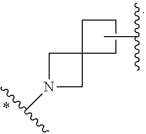

In some embodiments, ring A is.

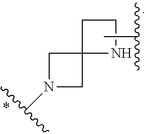

In some embodiments, ring A is

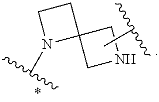

In some embodiments, ring A is

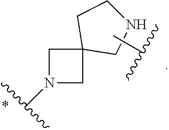

In some embodiments, ring A is

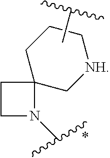

In some embodiments, ring A is

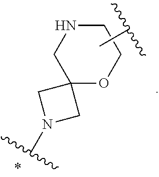

In some embodiments, ring A is

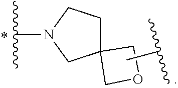

In some embodiments, ring A is

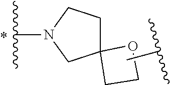

In some embodiments, ring A is

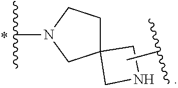

In some embodiments, ring A is

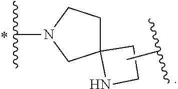

In some embodiments, ring A is

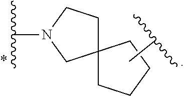

In some embodiments, ring A is

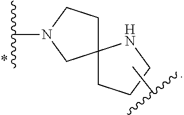

In some embodiments, ring A is

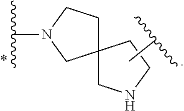

In some embodiments, ring A is

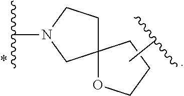

In some embodiments, ring A is

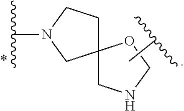

In some embodiments, ring A is

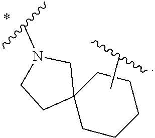

In some embodiments, ring A is

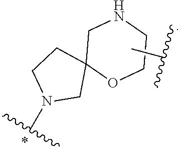

In some embodiments, ring A is

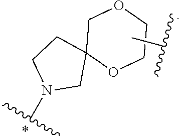

In some embodiments, ring A is

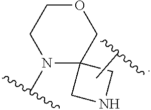

In some embodiments, ring A is

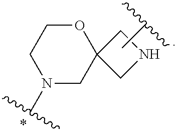

In some embodiments, ring A is

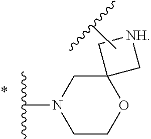

In some embodiments, ring A is

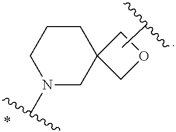

In some embodiments, ring A is

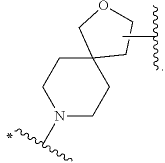

In some embodiments, ring A is

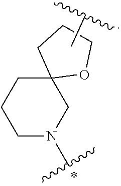

In some embodiments, ring A is

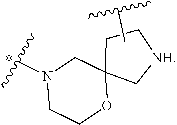

In some embodiments, ring A is

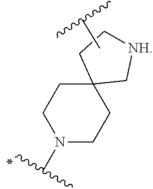

### Variable Ring B

In some embodiments, B is absent (B is H) or ring B is H, C₃-C₆ cycloalkyl, 4- to 10-membered heterocyclyl, C₆-C₁₀ aryl, or 5- to 10-membered heteroaryl.

In some embodiments, B is absent (B is H).

In some embodiments, ring B is C₃-C₆ cycloalkyl.

In some embodiments, ring B is C₃-C₆ monocyclic cycloalkyl.

In some embodiments, ring B is cyclopropyl, cyclobutyl, cyclopentyl, cyclohexyl, cycloheptyl, or cyclooctyl.

In some embodiments, ring B is C₃-C₆ bicyclic cycloalkyl.

In some embodiments, ring B is bicyclocyclobutyl, bicyclocyclopentyl, or bicyclocyclohexyl.

In some embodiments, ring B is C₄-C₆ fused bicyclic cycloalkyl.

In some embodiments, ring B is

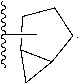

In some embodiments, ring B is

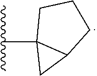 [SMILES: *C12CCCC1C2]

In some embodiments, ring B is

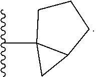 [SMILES: *C12CCCC1C2]

In some embodiments, ring B is C₅-C₆ bridged bicyclic cycloalkyl.

In some embodiments, ring B is

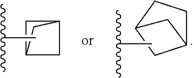

In some embodiments, ring B is

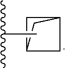

In some embodiments, ring B is

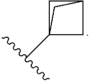 [SMILES: *C12CC(C1)C2]

In some embodiments, ring B is

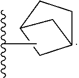

In some embodiments, ring B is

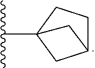 [SMILES: *C12CCC(C1)C2]

In some embodiments, ring B is C₅-C₆ spiro bicyclic cycloalkyl.

In some embodiments, ring B is spiro[2.2]pentyl or spiro[2.3]hexyl.

In some embodiments, ring B is

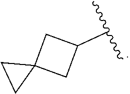 [SMILES: *C1CC2(CC2)C1]

In some embodiments, ring B is

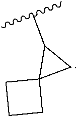 [SMILES: *C1CC12CCC2]

In some embodiments, ring B is 4- to 10-membered heterocyclyl.

In some embodiments, ring B is 4- to 10-membered monocyclic heterocyclyl.

In some embodiments, ring B is 4- to 10-membered bicyclic heterocyclyl.

In some embodiments, ring B is 4- to 10-membered fused bicyclic heterocyclyl.

In some embodiments, ring B is 5- to 10-membered bridged bicyclic heterocyclyl.

In some embodiments, ring B is 5- to 10-membered spiro bicyclic heterocyclyl.

In some embodiments, ring B is 4- to 10-membered heterocyclyl containing at least one heteroatom selected from N, O, and S.

In some embodiments, ring B is 4- to 10-membered monocyclic heterocyclyl containing at least one heteroatom selected from N, O, and S.

In some embodiments, ring B is 4- to 10-membered monocyclic heterocyclyl containing one N (e.g., azetidinyl, pyrrolidinyl, or piperidinyl).

In some embodiments, ring B is azetidinyl.

In some embodiments, ring B is

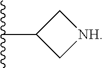 [SMILES: *C1CNC1]

In some embodiments, ring B is

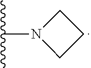 [SMILES: *N1CCC1]

In some embodiments, ring B is

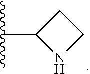 [SMILES: *C1CCN1]

In some embodiments, ring B is pyrrolidinyl.

In some embodiments, ring B is

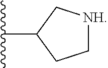 [SMILES: *C1CCNC1]

In some embodiments, ring B is

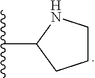 [SMILES: *C1CCCN1]

In some embodiments, ring B is

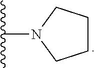 [SMILES: *N1CCCC1]

In some embodiments, ring B is piperidinyl.

In some embodiments, ring B is.

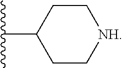 [SMILES: *C1CCNCC1]

In some embodiments, ring B is

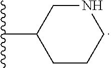 [SMILES: *C1CCCNC1]

In some embodiments, ring B is

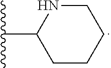 [SMILES: *C1CCCCN1]

In some embodiments, ring B is

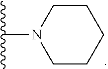 [SMILES: *N1CCCCC1]

In some embodiments, ring B is 4- to 10-membered fused bicyclic heterocyclyl containing one N.

In some embodiments, ring B is

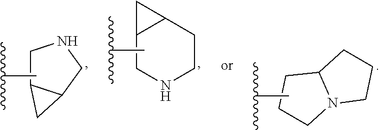

In some embodiments, ring B is

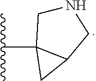 [SMILES: *C12CNCC1C2]

In some embodiments, ring B is

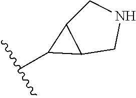 [SMILES: *C1C2CNCC12]

In some embodiments, ring B is

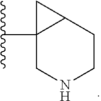 [SMILES: *C12CNCCC1C2]

In some embodiments, ring B is

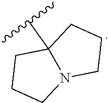 [SMILES: *C12CCCN1CCC2]

In some embodiments, ring B is 5- to 10-membered bridged bicyclic heterocyclyl containing one N.

In some embodiments, ring B is

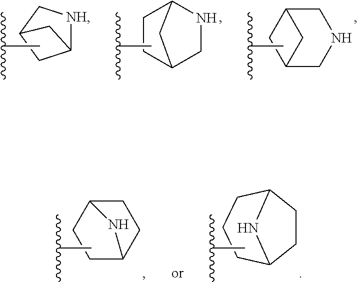

In some embodiments, ring B is

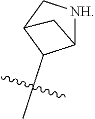 [SMILES: *C1C2CNC1C2]

In some embodiments, ring B is

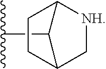 [SMILES: *C1C2CCC1NC2]

In some embodiments, ring B is

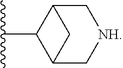 [SMILES: *C1C2CNCC1C2]

In some embodiments, ring B is

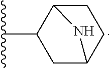 [SMILES: *C1CC2CCC1N2]

In some embodiments, ring B is

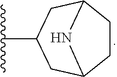 [SMILES: *C1CC2CCC(C1)N2]

In some embodiments, ring B is

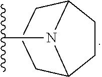 [SMILES: *N1C2CCCC1CC2]

In some embodiments, ring B is 5- to 10-membered spiro bicyclic heterocyclyl containing one N.

In some embodiments, ring B is

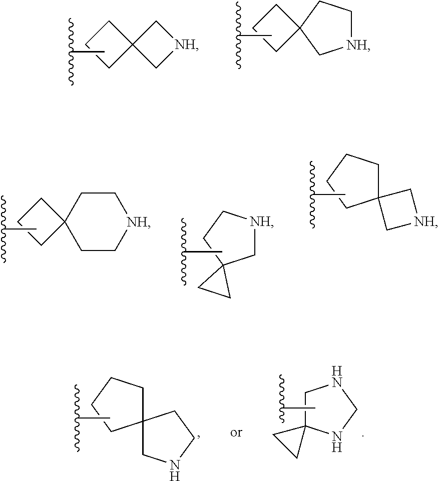

In some embodiments, ring B is

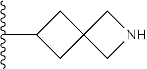 [SMILES: *C1CC2(CNC2)C1]

In some embodiments, ring B is

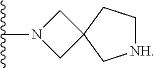 [SMILES: *N1CC2(CCNC2)C1]

In some embodiments, ring B is

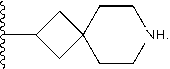 [SMILES: *C1CC2(CCNCC2)C1]

In some embodiments, ring B is

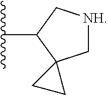 [SMILES: *C1CNCC12CC2]

In some embodiments, ring B is

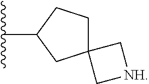 [SMILES: *C1CCC2(CNC2)C1]

In some embodiments, ring B is

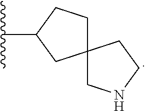 [SMILES: *C1CCC2(CCNC2)C1]

In some embodiments, ring B is 4- to 10-membered monocyclic heterocyclyl containing one O (e.g., oxetanyl, tetrahydrofuranyl, or tetrahydropyranyl).

In some embodiments, ring B is oxetanyl.

In some embodiments, ring B is

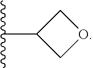 [SMILES: *C1COC1]

In some embodiments, ring B is

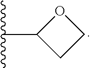 [SMILES: *C1CCO1]

In some embodiments, ring B is tetrahydrofuranyl.

In some embodiments, ring B is

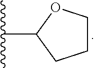 [SMILES: *C1CCCO1]

In some embodiments, ring B is

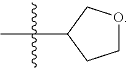 [SMILES: *C1CCOC1]

In some embodiments, ring B is tetrahydropyranyl.

In some embodiments, ring B is

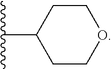 [SMILES: *C1CCOCC1]

In some embodiments, ring B is

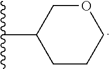 [SMILES: *C1CCCOC1]

In some embodiments, ring B is

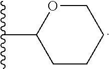 [SMILES: *C1CCCCO1]

In some embodiments, ring B is 5- to 10-membered bridged bicyclic heterocyclyl containing one O.

In some embodiments, ring B is 4- to 10-membered fused bicyclic heterocyclyl containing one O.

In some embodiments, ring B is 5- to 10-membered spiro bicyclic heterocyclyl containing one O.

In some embodiments, ring B is

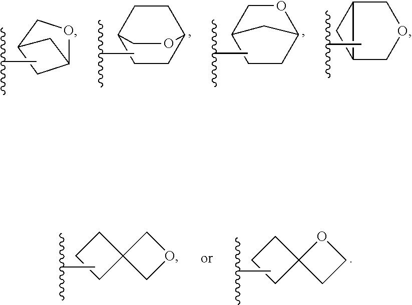

In some embodiments, ring B is

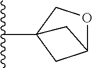 [SMILES: *C12COC(C1)C2]

In some embodiments, ring B is

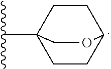 [SMILES: *C12CCC(CC1)OC2]

In some embodiments, ring B is

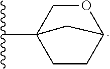 [SMILES: *C12CCC(C1)OC2]

In some embodiments, ring B is

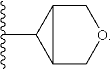 [SMILES: *C1C2COCC12]

In some embodiments, ring B is

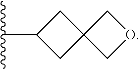 [SMILES: *C1CC2(COC2)C1]

In some embodiments, ring B is

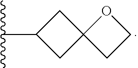 [SMILES: *C1CC2(CCO2)C1]

In some embodiments, ring B is 4- to 10-membered monocyclic heterocyclyl containing one S (e.g., tetrahydrothiophenyl).

In some embodiments, ring B is tetrahydrothiophenyl.

In some embodiments, ring B is

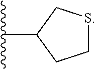 [SMILES: *C1CCSC1]

In some embodiments, ring B is

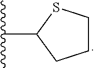 [SMILES: *C1CCCS1]

In some embodiments, ring B is 4- to 10-membered heterocyclyl (e.g., monocyclic, fused bicyclic, bridged bicyclic, or spiro bicyclic) containing two heteroatoms selected from N, O, and S.

In some embodiments, ring B is 4- to 10-membered monocyclic heterocyclyl containing two heteroatoms selected from N, O, and S.

In some embodiments, ring B is 4- to 10-membered fused bicyclic heterocyclyl containing two heteroatoms selected from N, O, and S.

In some embodiments, ring B is 5- to 10-membered bridged bicyclic heterocyclyl containing two heteroatoms selected from N, O, and S.

In some embodiments, ring B is 5- to 10-membered spiro bicyclic heterocyclyl containing two heteroatoms selected from N, O, and S.

In some embodiments, ring B is

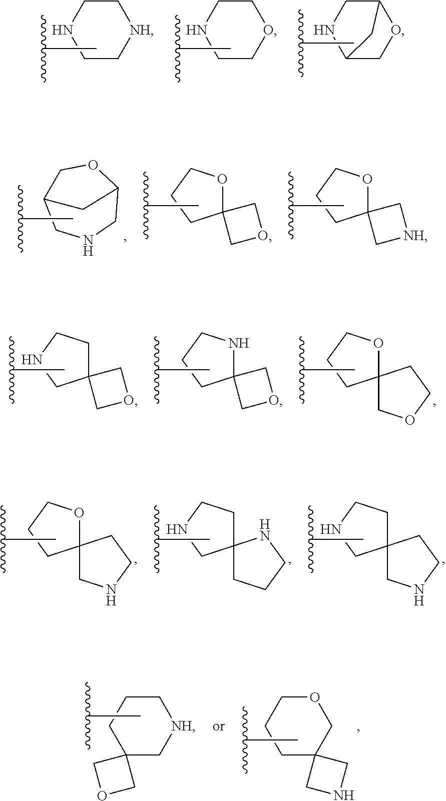

In some embodiments, ring B is

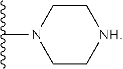 [SMILES: *N1CCNCC1]

In some embodiments, ring B is

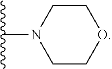 [SMILES: *N1CCOCC1]

In some embodiments, ring B is

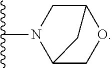 [SMILES: *N1CC2CC1CO2]

In some embodiments, ring B is

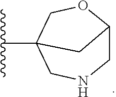 [SMILES: *C12CNCC(C1)OC2]

In some embodiments, ring B is

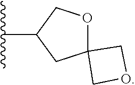 [SMILES: *C1COC2(COC2)C1]

In some embodiments, ring B is

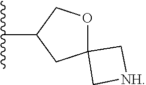 [SMILES: *C1COC2(CNC2)C1]

In some embodiments, ring B is

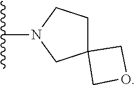 [SMILES: *N1CCC2(COC2)C1]

In some embodiments, ring B is

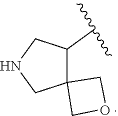 [SMILES: *C1CNCC12COC2]

In some embodiments, ring B is

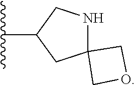 [SMILES: *C1CNC2(COC2)C1]

In some embodiments, ring B is

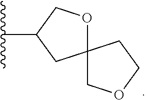 [SMILES: *C1COC2(CCOC2)C1]

In some embodiments, ring B is

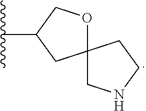 [SMILES: *C1COC2(CCNC2)C1]

In some embodiments, ring B is

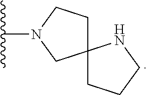 [SMILES: *N1CCC2(CCCN2)C1]

In some embodiments, ring B is

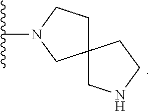 [SMILES: *N1CCC2(CCNC2)C1]

In some embodiments, ring B is

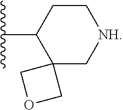 [SMILES: *C1CCNCC12COC2]

In some embodiments, ring B is

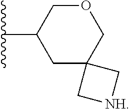 [SMILES: *C1COCC2(CNC2)C1]

In some embodiments, ring B is C₆-C₁₀ aryl (e.g., phenyl).

In some embodiments, ring B is phenyl.

In some embodiments, ring B is 5- to 10-membered heteroaryl.

In some embodiments, ring B is 5- to 10-membered heteroaryl containing at least one heteroatom selected from N, O, and S.

In some embodiments, ring B is 5-membered heteroaryl containing at least one heteroatom selected from N, O, and S.

In some embodiments, ring B is 5-membered heteroaryl containing one heteroatom selected from N, O, and S (e.g., pyrrolyl, furanyl, or thiophenyl).

In some embodiments, ring B is 5-membered heteroaryl containing two heteroatoms selected from N, O, and S (e.g., oxazolyl, thiazolyl, pyrazolyl, isoxazolyl, isothiazolyl, or imidazolyl).

In some embodiments, ring B is oxazolyl.

In some embodiments, ring B is

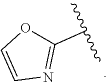 [SMILES: *c1ncco1]

In some embodiments, ring B is thiazolyl.

In some embodiments, ring B is

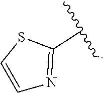 [SMILES: *c1nccs1]

In some embodiments, ring B is pyrazolyl.

In some embodiments, ring B is

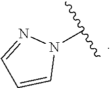 [SMILES: *n1cccn1]

In some embodiments, ring B is

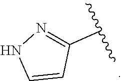 [SMILES: *c1cc[nH]n1]

In some embodiments, ring B is isoxazolyl.

In some embodiments, ring B is isothiazolyl.

In some embodiments, ring B is imidazolyl.

In some embodiments, ring B is 5-membered heteroaryl containing three heteroatoms selected from N, O, and S (e.g., triazolyl, oxadiazolyl, thiadiazolyl, or oxathiadiazolyl).

In some embodiments, ring B is triazolyl.

In some embodiments, ring B is

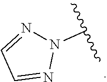 [SMILES: *n1nccn1]

In some embodiments, ring B is

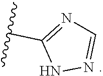 [SMILES: *c1ncn[nH]1]

In some embodiments, ring B is

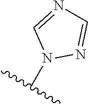 [SMILES: *n1cncn1]

In some embodiments, ring B is oxadiazolyl.

In some embodiments, ring B is

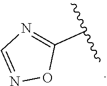 [SMILES: *c1ncno1]

In some embodiments, ring B is thiadiazolyl.

In some embodiments, ring B is

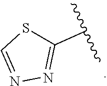 [SMILES: *c1nncs1]

In some embodiments, ring B is

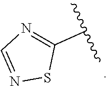 [SMILES: *c1ncns1]

In some embodiments, ring B is 5-membered heteroaryl containing four heteroatoms selected from N, O, and S (e.g., tetrazolyl or oxatriazolyl).

In some embodiments, ring B is tetrazolyl.

In some embodiments, ring B is

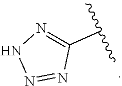 [SMILES: *c1nn[nH]n1]

In some embodiments, ring B is oxatriazolyl.

In some embodiments, ring B is 6-membered heteroaryl containing at least one heteroatom selected from N, O, and S.

In some embodiments, ring B is 6-membered heteroaryl containing one heteroatom selected from N, O, and S (e.g., pyridinyl, pyranyl, or thiopyranyl).

In some embodiments, ring B is pyridinyl.

In some embodiments, ring B is

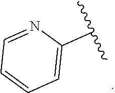 [SMILES: *c1ccccn1]

In some embodiments, ring B is 6-membered heteroaryl containing two heteroatoms selected from N, O, and S (e.g., pyrimidinyl, pyridazinyl, or pyrazinyl).

In some embodiments, ring B is pyrimidinyl.

In some embodiments, ring B is

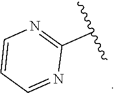 [SMILES: *c1ncccn1]

In some embodiments, ring B is

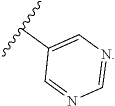 [SMILES: *c1cncnc1]

In some embodiments, ring B is pyridazinyl.

In some embodiments, ring B is

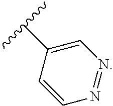 [SMILES: *c1ccnnc1]

In some embodiments, ring B is pyrazinyl.

In some embodiments, ring B is 9-membered heteroaryl containing at least one heteroatom selected from N, O, and S.

In some embodiments, ring B is

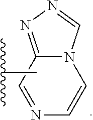

In some embodiments, ring B is

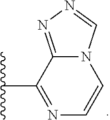 [SMILES: *c1nccn2cnnc12]

In some embodiments, ring B is C₃-C₆ cycloalkyl, 4- to 7-membered heterocyclyl, phenyl, or 5- to 6-membered heteroaryl.

In some embodiments, ring B is pyridinyl, pyranyl, thiopyranyl, pyrimidinyl, pyridazinyl, or pyrazinyl.

### Variable Z

In some embodiments, Z is absent, —CH₂—, —O—, or —C(═O)—.

In some embodiments, Z is absent.

In some embodiments, Z is —CH₂—.

In some embodiments, Z is —O—.

In some embodiments, Z is —C(═O)—.

### Variable E

In some embodiments, E is —(C₁-C₆ alkylene)- optionally substituted with one or more oxo, halogen, cyano, —OH, —O(C₁-C₆ alkyl), —NH₂, —NH(C₁-C₆ alkyl), or —N(C₁-C₆ alkyl)₂.

In some embodiments, E is —(C₁-C₆ alkylene)- (e.g., -methylene-, -ethylene-, -n-propylene-, -isopropylene-, -n-butylene-, -t-butylene-, -isobutylene-, or -sec-butylene-).

In some embodiments, E is —(C₁-C₆ alkylene)- (e.g., -methylene-, -ethylene-, -n-propylene-, -isopropylene-, -n-butylene-, -t-butylene-, -isobutylene-, or -sec-butylene-) substituted with one or more oxo, halogen, cyano, —OH, —O(C₁-C₆ alkyl), —NH₂, —NH(C₁-C₆ alkyl), or —N(C₁-C₆ alkyl)₂.

In some embodiments, E is -methylene- substituted with one or more —NH₂.

Variables U¹, U², and R^{U}

In some embodiments, one of U¹ and U² is —O—, and the other one of U¹ and U² is —C(R^{U})₂—.

In some embodiments, U¹ is —O—, and U² is —C(R^{U})₂—.

In some embodiments, U² is —O—, and U¹—C(R^{U})₂—.

In some embodiments, at least one R^{U} is H, halogen, cyano, —OH, —O(C₁-C₆ alkyl), —NH₂, —NH(C₁-C₆ alkyl), —N(C₁-C₆ alkyl)₂, or C₁-C₆ alkyl.

In some embodiments, at least one R^{U} is H.

In some embodiments, at least one R^{U} is halogen (e.g., —F, —Cl, —Br, or —I).

In some embodiments, at least one R^{U} is cyano.

In some embodiments, at least one R^{U} is —OH

In some embodiments, at least one R^{U} is —O(C₁-C₆ alkyl) (e.g., —O(methyl), —O(ethyl), —O(n-propyl), —O(isopropyl), —O(n-butyl), —O(t-butyl), —O(isobutyl), or —O(sec-butyl)).

In some embodiments, at least one R^{U} is —NH₂.

In some embodiments, at least one R^{U} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{U} is —N(C₁-C₆ alkyl)₂ (e.g., e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, at least one R^{U} is C₁-C₆ alkyl (e.g., methyl, ethyl, n-propyl, isopropyl, n-butyl, t-butyl, isobutyl, or sec-butyl).

In some embodiments, at least one R^{U} is methyl.

In some embodiments, at least one R^{U} is ethyl.

In some embodiments, at least one R^{U} is n-propyl.

In some embodiments, at least one R^{U} is isopropyl.

In some embodiments, at least one R^{U} is n-butyl.

Variables R^{V}, R^{Va}, R^{Vb}, R^{Vc}, R^{A}, R^{B}, and R^{C}

### Variable R^{V}

In some embodiments, at least one R^{V} is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—(R^{A}), —C(═O)—H, —C(═O)—(R^{A}), —C(═O)—OH, —C(═O)—O(R^{A}), —C(═O)—NH₂, —C(═O)—NH(R^{A}), —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Va}.

In some embodiments, at least one R^{V} is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—(R^{A}), —C(═O)—H, —C(═O)—(R^{A}), —C(═O)—OH, —C(═O)—O(R^{A}), —C(═O)—NH₂, —C(═O)—NH(R^{A}), —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{V} is H, oxo, halogen, cyano, —OH, —OR^{A}, —(C₁-C₆ alkylene)-OR^{A}, —NH₂, —NHR^{A}, —N(R^{A})₂, —NH—C(═O)—H, —NH—C(═O)—(R^{A}), —C(═O)—H, —C(═O)—(R^{A}), —C(═O)—OH, —C(═O)—O(R^{A}), —C(═O)—NH₂, —C(═O)—NH(R^{A}), —C(═O)—N(R^{A})₂, —S(═O)₂—R^{A}, —NH—S(═O)₂—R^{A}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{Va}.

In some embodiments, at least one R^{V} is C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Va}.

In some embodiments, at least one R^{V} is C₁-C₆ alkyl, C₂-C₆ alkenyl, C₂-C₆ alkynyl, monocyclic C₃-C₈ cycloalkyl, fused bicyclic C₄-C₈ cycloalkyl, bridged bicyclic C₅-C₈ cycloalkyl, spiro bicyclic C₅-C₈ cycloalkyl, monocyclic 4- to 8-membered heterocyclyl, fused bicyclic 4- to 10-membered heterocyclyl, bridged bicyclic 5- to 10-membered heterocyclyl, spiro bicyclic 5- to 10-membered heterocyclyl, phenyl, monocyclic 5- to 6-membered heteroaryl, fused bicyclic 5- to 10-membered heteroaryl, —(C₁-C₄ alkyl)-(monocyclic C₃-C₈ cycloalkyl), —(C₁-C₄ alkyl)-(fused bicyclic C₄-C₈ cycloalkyl), —(C₁-C₄ alkyl)-(bridged bicyclic C₅-C₈ cycloalkyl), —(C₁-C₄ alkyl)-(spiro bicyclic C₅-C₈ cycloalkyl), —(C₁-C₄ alkyl)-(monocyclic 4- to 7-membered heterocyclyl), —(C₁-C₄ alkyl)-(fused bicyclic 4- to 10-membered heterocyclyl), —(C₁-C₄ alkyl)-(bridged bicyclic 5- to 10-membered heterocyclyl), —(C₁-C₄ alkyl)-(spiro bicyclic 5- to 10-membered heterocyclyl), —(C₁-C₄ alkyl)-(phenyl), —(C₁-C₄ alkyl)-(monocyclic 5- to 6-membered heteroaryl), or —(C₁-C₄ alkyl)-(fused bicyclic 5- to 10-membered heteroaryl), each optionally substituted with one or more R^{Va}.

In some embodiments, at least one R^{V} is H.

In some embodiments, at least one R^{V} is oxo.

In some embodiments, at least one R^{V} is halogen (e.g., —F, —Cl, —Br, —I).

In some embodiments, at least one R^{V} is cyano.

In some embodiments, at least one R^{V} is —OH.

In some embodiments, at least one R^{V} is —OR^{A}.

In some embodiments, at least one R^{V} is —(C₁-C₆ alkylene)-OR^{A}.

In some embodiments, at least one R^{V} is —NH₂.

In some embodiments, at least one R^{V} is —NHR^{A}.

In some embodiments, at least one R^{V} is —N(R^{A})₂.

In some embodiments, at least one R^{V} is —NH—C(═O)—H.

In some embodiments, at least one R^{V} is —NH—C(═O)—R^{A}.

In some embodiments, at least one R^{V} is —C(═O)—H.

In some embodiments, at least one R^{V} is —C(═O)—R^{A}.

In some embodiments, at least one R^{V} is —C(═O)—OH.

In some embodiments, at least one R^{V} is —C(═O)—OR^{A}.

In some embodiments, at least one R^{V} is —C(═O)—NH₂.

In some embodiments, at least one R^{V} is —C(═O)—NHR^{A}.

In some embodiments, at least one R^{V} is —C(═O)—N(R^{A})₂.

In some embodiments, at least one R^{V} is —S(═O)₂—R^{A}.

In some embodiments, at least one R^{V} is —NH—S(═O)₂—R^{A}.

In some embodiments, at least one R^{V} is C₁-C₆ alkyl (e.g., methyl, ethyl, n-propyl, isopropyl, n-butyl, t-butyl, isobutyl, or sec-butyl).

In some embodiments, at least one R^{V} is C₁-C₆ alkyl (e.g., methyl, ethyl, n-propyl, isopropyl, n-butyl, t-butyl, isobutyl, or sec-butyl) substituted with one or more R^{Va}.

In some embodiments, at least one R^{V} is methyl.

In some embodiments, at least one R^{V} is ethyl.

In some embodiments, at least one R^{V} is n-propyl.

In some embodiments, at least one R^{V} is isopropyl.

In some embodiments, at least one R^{V} is n-butyl.

In some embodiments, at least one R^{V} is C₂-C₆ alkenyl.

In some embodiments, at least one R^{V} is C₂-C₆ alkenyl substituted with one or more R^{Va}.

In some embodiments, at least one R^{V} is C₂-C₆ alkynyl.

In some embodiments, at least one R^{V} is C₂-C₆ alkynyl substituted with one or more R^{Va}.

In some embodiments, at least one R^{V} is C₃-C₆ cycloalkyl.

In some embodiments, at least one R^{V} is C₃-C₆ cycloalkyl substituted with one or more R^{Va}.

In some embodiments, at least one R^{V} is C₃-C₆ monocyclic cycloalkyl.

In some embodiments, at least one R^{V} is C₃-C₆ monocyclic cycloalkyl substituted with one or more R^{Va}.

In some embodiments, at least one R^{V} is cyclopropyl, cyclobutyl, cyclopentyl, cyclohexyl, cycloheptyl, or cyclooctyl.

In some embodiments, at least one R^{V} is C₃-C₆ bicyclic cycloalkyl.

In some embodiments, at least one R^{V} is C₃-C₆ bicyclic cycloalkyl substituted with one or more R^{Va}.

In some embodiments, at least one R^{V} is bicyclocyclobutyl, bicyclocyclopentyl, or bicyclocyclohexyl.

In some embodiments, at least one R^{V} is C₄-C₆ fused bicyclic cycloalkyl.

In some embodiments, at least one R^{V} is C₄-C₆ fused bicyclic cycloalkyl substituted with one or more R^{Va}.

In some embodiments, at least one R^{V} is

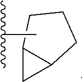

In some embodiments, at least one R^{V} is

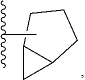

substituted with one or more R^{Va}.

In some embodiments, at least one R^{V} is

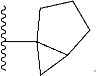 [SMILES: *C12CCCC1C2]

In some embodiments, at least one R^{V} is

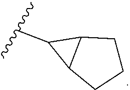 [SMILES: *C1C2CCCC12]

In some embodiments, at least one R^{V} is C₅-C₆ bridged bicyclic cycloalkyl.

In some embodiments, at least one R^{V} is C₅-C₆ bridged bicyclic cycloalkyl substituted with one or more R^{Va}.

In some embodiments, at least one R^{V} is

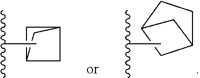

In some embodiments, at least one R^{V} is

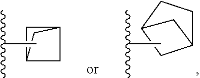

substituted with one or more R^{Va}.

In some embodiments, at least one R^{V} is

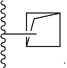

In some embodiments, at least one R^{V} is

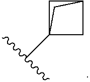 [SMILES: *C12CC(C1)C2]

In some embodiments, at least one R^{V} is

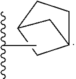

In some embodiments, at least one R^{V} is

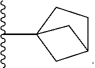 [SMILES: *C12CCC(C1)C2]

In some embodiments, at least one R^{V} is C₅-C₆ spiro bicyclic cycloalkyl.

In some embodiments, at least one R^{V} is C₅-C₆ spiro bicyclic cycloalkyl substituted with one or more R^{Va}.

In some embodiments, at least one R^{V} is spiro[2.2]pentyl or spiro[2.3]hexyl.

In some embodiments, at least one R^{V} is spiro[2.2]pentyl or spiro[2.3]hexyl, substituted with one or more R^{Va}.

In some embodiments, at least one R^{V} is

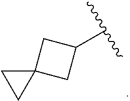 [SMILES: *C1CC2(CC2)C1]

In some embodiments, at least one R^{V} is

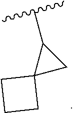 [SMILES: *C1CC12CCC2]

In some embodiments, at least one R^{V} is 4- to 10-membered heterocyclyl.

In some embodiments, at least one R^{V} is 4- to 10-membered heterocyclyl substituted with one or more R^{Va}.

In some embodiments, at least one R^{V} is 4- to 10-membered monocyclic heterocyclyl.

In some embodiments, at least one R^{V} is 4- to 10-membered monocyclic heterocyclyl substituted with one or more R^{Va}.

In some embodiments, at least one R^{V} is 4- to 10-membered bicyclic heterocyclyl.

In some embodiments, at least one R^{V} is 4- to 10-membered bicyclic heterocyclyl substituted with one or more R^{Va}.

In some embodiments, at least one R^{V} is 4- to 10-membered fused bicyclic heterocyclyl.

In some embodiments, at least one R^{V} is 4- to 10-membered fused bicyclic heterocyclyl substituted with one or more R^{Va}.

In some embodiments, at least one R^{V} is 5- to 10-membered bridged bicyclic heterocyclyl.

In some embodiments, at least one R^{V} is 5- to 10-membered bridged bicyclic heterocyclyl substituted with one or more R^{Va}.

In some embodiments, at least one R^{V} is 5- to 10-membered spiro bicyclic heterocyclyl.

In some embodiments, at least one R^{V} is 5- to 10-membered spiro bicyclic heterocyclyl substituted with one or more R^{Va}.

In some embodiments, at least one R^{V} is 4- to 10-membered heterocyclyl containing at least one heteroatom selected from N, O, and S.

In some embodiments, at least one R^{V} is 4- to 10-membered heterocyclyl containing at least one heteroatom selected from N, O, and S, wherein the 4- to 10-membered heterocyclyl is substituted with one or more R^{Va}.

In some embodiments, at least one R^{V} is 4- to 10-membered monocyclic heterocyclyl containing at least one heteroatom selected from N, O, and S.

In some embodiments, at least one R^{V} is 4- to 10-membered monocyclic heterocyclyl containing at least one heteroatom selected from N, O, and S, wherein the 4- to 10-membered heterocyclyl is substituted with one or more R^{Va}.

In some embodiments, at least one R^{V} is 4- to 10-membered monocyclic heterocyclyl containing one N (e.g., azetidinyl, pyrrolidinyl, or piperidinyl).

In some embodiments, at least one R^{V} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Va}.

In some embodiments, at least one R^{V} is azetidinyl.

In some embodiments, at least one R^{V} is azetidinyl substituted with one or more R^{Va}.

In some embodiments, at least one R^{V} is

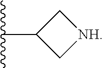 [SMILES: *C1CNC1]

In some embodiments, at least one R^{V} is

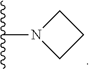 [SMILES: *N1CCC1]

In some embodiments, at least one R is

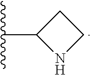 [SMILES: *C1CCN1]

In some embodiments, at least one R^{V} is pyrrolidinyl.

In some embodiments, at least one R^{V} is

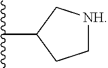 [SMILES: *C1CCNC1]

In some embodiments, at least one R^{V} is

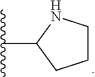 [SMILES: *C1CCCN1]

In some embodiments, at least one R^{V} is

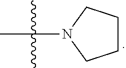 [SMILES: *N1CCCC1]

In some embodiments, at least one R^{V} is piperidinyl.

In some embodiments, at least one R^{V} is

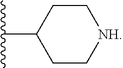 [SMILES: *C1CCNCC1]

In some embodiments, at least one R^{V} is

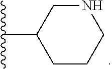 [SMILES: *C1CCCNC1]

In some embodiments, at least one R^{V} is

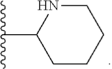 [SMILES: *C1CCCCN1]

In some embodiments, at least one R^{V} is

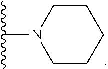 [SMILES: *N1CCCCC1]

In some embodiments, at least one R^{V} is 4- to 10-membered fused bicyclic heterocyclyl containing one N.

In some embodiments, at least one R^{V} is 4- to 10-membered fused bicyclic heterocyclyl containing one N, wherein the 4- to 10-membered fused bicyclic heterocyclyl is substituted with one or more R^{Va}.

In some embodiments, at least one R^{V} is

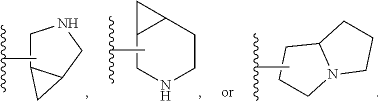

In some embodiments, at least one R^{V} is

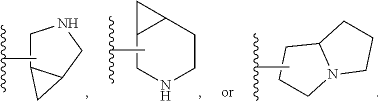

substituted with one or more R^{Va}.

In some embodiments, at least one R^{V} is

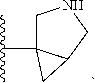 [SMILES: *C12CNCC1C2]

In some embodiments, at least one R^{V} is

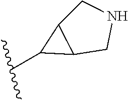 [SMILES: *C1C2CNCC12]

In some embodiments, at least one R^{V} is

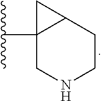 [SMILES: *C12CNCCC1C2]

In some embodiments, at least one R^{V} is

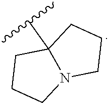 [SMILES: *C12CCCN1CCC2]

In some embodiments, at least one R^{V} is 5- to 10-membered bridged bicyclic heterocyclyl containing one N.

In some embodiments, at least one R^{V} is 5- to 10-membered bridged bicyclic heterocyclyl containing one N, wherein the 5- to 10-membered bridged bicyclic heterocyclyl is substituted with one or more R^{Va}.

In some embodiments, at least one R^{V} is

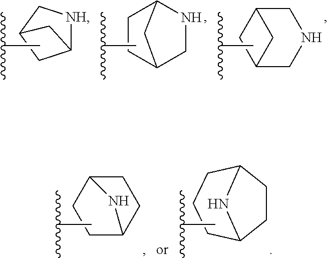

In some embodiments, at least one R^{V} is

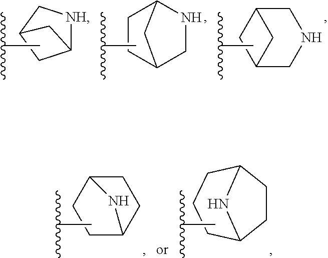

substituted with one or more R^{Va}.

In some embodiments, at least one R^{V} is

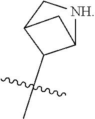 [SMILES: *C1C2CNC1C2]

In some embodiments, at least one R is

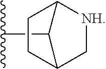 [SMILES: *C1C2CCC1NC2]

In some embodiments, at least one R^{V} is

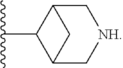 [SMILES: *C1C2CNCC1C2]

In some embodiments, at least one R^{V} is

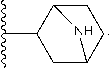 [SMILES: *C1CC2CCC1N2]

In some embodiments, at least one R^{V} is

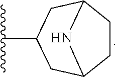 [SMILES: *C1CC2CCC(C1)N2]

In some embodiments, at least one R^{V} is

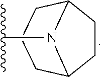 [SMILES: *N1C2CCCC1CC2]

In some embodiments, at least one R^{V} is 5- to 10-membered spiro bicyclic heterocyclyl containing one N.

In some embodiments, at least one R^{V} is 5- to 10-membered spiro bicyclic heterocyclyl containing one N, wherein the 5- to 10-membered spiro bicyclic heterocyclyl is substituted with one or more R^{Va}.

In some embodiments, at least one R^{V} is

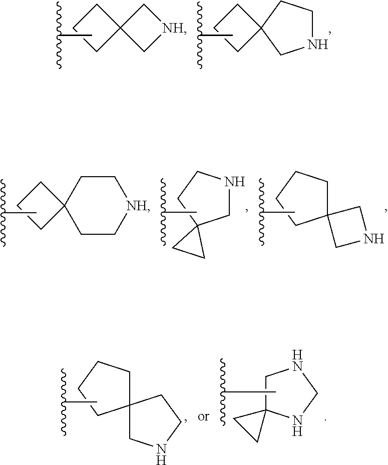

In some embodiments, at least one R^{V} is

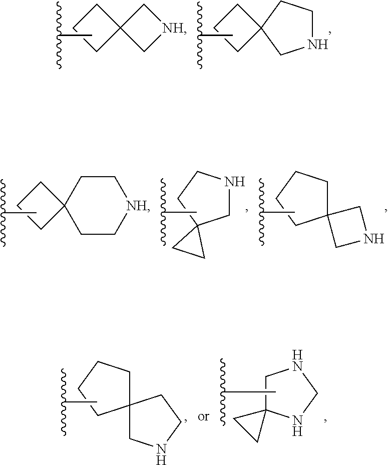

substituted with one or more R^{Va}.

In some embodiments, at least one R^{V} is

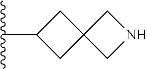 [SMILES: *C1CC2(CNC2)C1]

In some embodiments, at least one R^{V} is

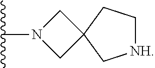 [SMILES: *N1CC2(CCNC2)C1]

In some embodiments, at least one R^{V} is

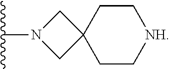 [SMILES: *N1CC2(CCNCC2)C1]

In some embodiments, at least one R^{V} is

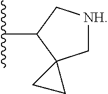 [SMILES: *C1CNCC12CC2]

In some embodiments, at least one R^{V} is

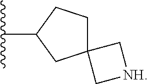 [SMILES: *C1CCC2(CNC2)C1]

In some embodiments, at least one R^{V} is

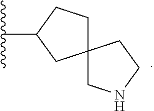 [SMILES: *C1CCC2(CCNC2)C1]

In some embodiments, at least one R^{V} is 4- to 10-membered monocyclic heterocyclyl containing one O (e.g., oxetanyl, tetrahydrofuranyl, or tetrahydropyranyl).

In some embodiments, at least one R^{V} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Va}.

In some embodiments, at least one R^{V} is oxetanyl.

In some embodiments, at least one R^{V} is

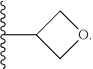 [SMILES: *C1COC1]

In some embodiments, at least one R^{V} is

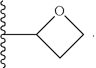 [SMILES: *C1CCO1]

In some embodiments, at least one R^{V} is tetrahydrofuranyl.

In some embodiments, at least one R^{V} is

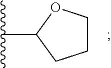 [SMILES: *C1CCCO1]

In some embodiments, at least one R^{V} is

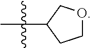 [SMILES: *C1CCOC1]

In some embodiments, at least one R^{V} is tetrahydropyranyl.

In some embodiments, at least one R^{V} is.

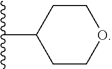 [SMILES: *C1CCOCC1]

In some embodiments, at least one R^{V} is

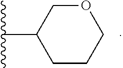 [SMILES: *C1CCCOC1]

In some embodiments, at least one R^{V} is

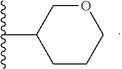 [SMILES: *C1CCCOC1]

In some embodiments, at least one R^{V} is 5- to 10-membered bridged bicyclic heterocyclyl containing one O.

In some embodiments, at least one R^{V} is 5- to 10-membered bridged bicyclic heterocyclyl containing one O, wherein the 5- to 10-membered bridged bicyclic heterocyclyl is substituted with one or more R^{Va}.

In some embodiments, at least one R^{V} is 4- to 10-membered fused bicyclic heterocyclyl containing one O.

In some embodiments, at least one R^{V} is 4- to 10-membered fused bicyclic heterocyclyl containing one O, wherein the 4- to 10-membered fused heterocyclyl is substituted with one or more R^{Va}.

In some embodiments, at least one R^{V} is 5- to 10-membered spiro bicyclic heterocyclyl containing one O.

In some embodiments, at least one R^{V} is 5- to 10-membered spiro bicyclic heterocyclyl containing one O, wherein the is 5- to 10-membered spiro bicyclic heterocyclyl is substituted with one or more R^{Va}.

In some embodiments, at least one R^{V} is

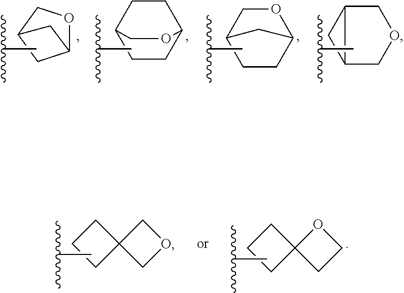

In some embodiments, at least one R^{V} is

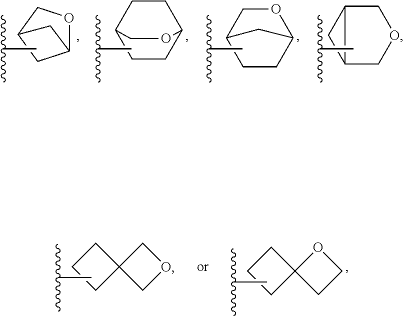

substituted with one or more R^{Va}.

In some embodiments, at least one R^{V} is

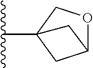 [SMILES: *C12COC(C1)C2]

In some embodiments, at least one R^{V} is

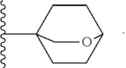 [SMILES: *C12CCC(CC1)OC2]

In some embodiments, at least one R^{V} is

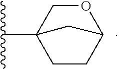 [SMILES: *C12CCC(C1)OC2]

In some embodiments, at least one R^{V} is

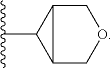 [SMILES: *C1C2COCC12]

In some embodiments, at least one R^{V} is

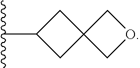 [SMILES: *C1CC2(COC2)C1]

In some embodiments, at least one R^{V} is

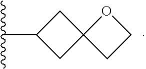 [SMILES: *C1CC2(CCO2)C1]

In some embodiments, at least one R^{V} is 4- to 10-membered monocyclic heterocyclyl containing one S (e.g., tetrahydrothiophenyl).

In some embodiments, at least one R^{V} is 4- to 10-membered monocyclic heterocyclyl containing one S (e.g., tetrahydrothiophenyl), wherein the 4- to 10-membered monocyclic heterocyclyl is substituted with one or more R^{Va}.

In some embodiments, at least one R^{V} is tetrahydrothiophenyl.

In some embodiments, at least one R^{V} is

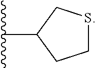 [SMILES: *C1CCSC1]

In some embodiments, at least one R^{V} is

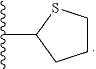 [SMILES: *C1CCCS1]

In some embodiments, at least one R^{V} is 4- to 10-membered heterocyclyl (e.g., monocyclic, fused bicyclic, bridged bicyclic, or spiro bicyclic) containing two heteroatoms selected from N, O, and S.

In some embodiments, at least one R^{V} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Va}.

In some embodiments, at least one R^{V} is 4- to 10-membered monocyclic heterocyclyl containing two heteroatoms selected from N, O, and S.

In some embodiments, at least one R^{V} is 4- to 10-membered monocyclic heterocyclyl containing two heteroatoms selected from N, O, and S, wherein the 4- to 10-membered monocyclic heterocyclyl is substituted with one or more R^{Va}.

In some embodiments, at least one R^{V} is 4- to 10-membered fused bicyclic heterocyclyl containing two heteroatoms selected from N, O, and S.

In some embodiments, at least one R^{V} is 4- to 10-membered fused bicyclic heterocyclyl containing two heteroatoms selected from N, O, and S, wherein the 4- to 10-membered fused bicyclic heterocyclyl is substituted with one or more R^{Va}.

In some embodiments, at least one R^{V} is 5- to 10-membered bridged bicyclic heterocyclyl containing two heteroatoms selected from N, O, and S.

In some embodiments, at least one R^{V} is 5- to 10-membered bridged bicyclic heterocyclyl containing two heteroatoms selected from N, O, and S, wherein the 5- to 10-membered bridged bicyclic heterocyclyl is substituted with one or more R^{Va}.

In some embodiments, at least one R^{V} is 5- to 10-membered spiro bicyclic heterocyclyl containing two heteroatoms selected from N, O, and S.

In some embodiments, at least one R^{V} is 5- to 10-membered spiro bicyclic heterocyclyl containing two heteroatoms selected from N, O, and S, wherein the 5- to 10-membered spiro bicyclic heterocyclyl is substituted with one or more R^{Va}.

In some embodiments, at least one R^{V} is

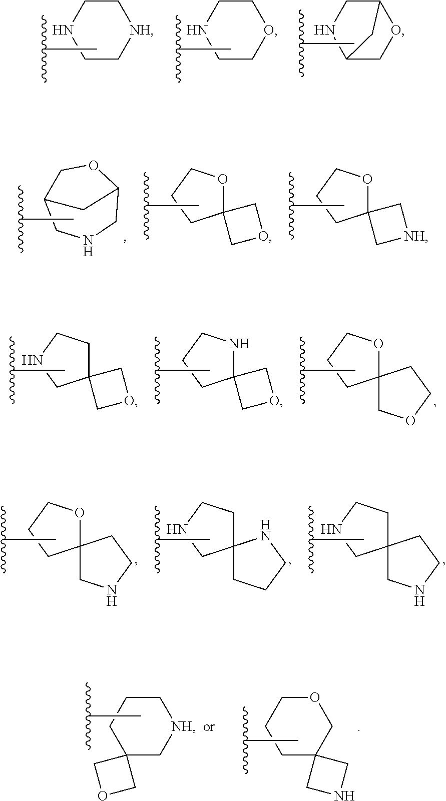

In some embodiments, at least one R^{V} is

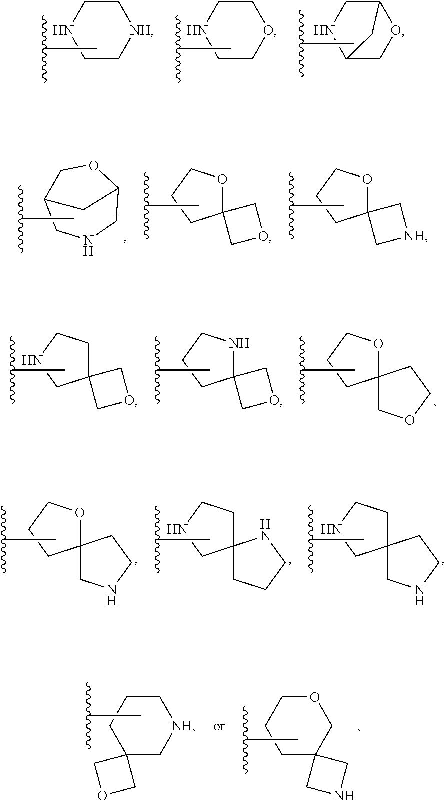

substituted with one or more R^{Va}.

In some embodiments, at least one R^{V} is

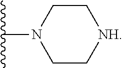 [SMILES: *N1CCNCC1]

In some embodiments, at least one R^{V} is

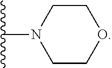 [SMILES: *N1CCOCC1]

In some embodiments, at least one R^{V} is

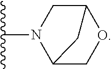 [SMILES: *N1CC2CC1CO2]

In some embodiments, at least one R^{V} is H

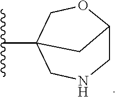 [SMILES: *C12CNCC(C1)OC2]

In some embodiments, at least one R^{V} is

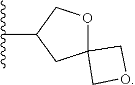 [SMILES: *C1COC2(COC2)C1]

In some embodiments, at least one R^{V} is

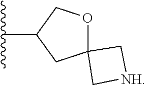 [SMILES: *C1COC2(CNC2)C1]

In some embodiments, at least one R^{V} is

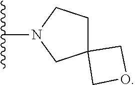 [SMILES: *N1CCC2(COC2)C1]

In some embodiments, at least one R^{V} is

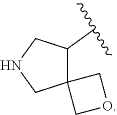 [SMILES: *C1CNCC12COC2]

In some embodiments, at least one R^{V} is

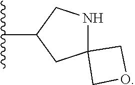 [SMILES: *C1CNC2(COC2)C1]

In some embodiments, at least one R^{V} is

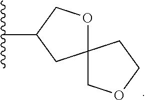 [SMILES: *C1COC2(CCOC2)C1]

In some embodiments, at least one R^{V} is

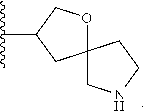 [SMILES: *C1COC2(CCNC2)C1]

In some embodiments, at least one R^{c} is

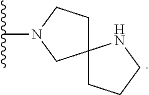 [SMILES: *N1CCC2(CCCN2)C1]

In some embodiments, at least one R^{V} is

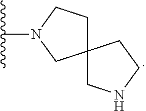 [SMILES: *N1CCC2(CCNC2)C1]

In some embodiments, at least one R^{V} is

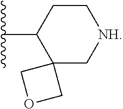 [SMILES: *C1CCNCC12COC2]

In some embodiments, at least one R^{V} is

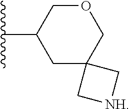 [SMILES: *C1COCC2(CNC2)C1]

In some embodiments, at least one R^{V} is C₆-C₁₀ aryl (e.g., phenyl).

In some embodiments, at least one R^{V} is C₆-C₁₀ aryl (e.g., phenyl) substituted with one or more R^{Va}.

In some embodiments, at least one R^{V} is phenyl.

In some embodiments, at least one R^{V} is 5- to 10-membered heteroaryl.

In some embodiments, at least one R^{V} is 5- to 10-membered heteroaryl substituted with one or more R^{Va}.

In some embodiments, at least one R^{V} is 5- to 10-membered heteroaryl containing at least one heteroatom selected from N, O, and S.

In some embodiments, at least one R^{V} is 5- to 10-membered heteroaryl containing at least one heteroatom selected from N, O, and S, wherein the heteroaryl is substituted with one or more R^{Va}.

In some embodiments, at least one R^{V} is 5-membered heteroaryl containing at least one heteroatom selected from N, O, and S.

In some embodiments, at least one R^{V} is 5-membered heteroaryl containing at least one heteroatom selected from N, O, and S, wherein the heteroaryl is substituted with one or more R^{Va}.

In some embodiments, at least one R^{V} is 5-membered heteroaryl containing one heteroatom selected from N, O, and S (e.g., pyrrolyl, furanyl, or thiophenyl).

In some embodiments, at least one R^{V} is 5-membered heteroaryl containing one heteroatom selected from N, O, and S (e.g., pyrrolyl, furanyl, or thiophenyl), wherein the heteroaryl is substituted with one or more R^{Va}.

In some embodiments, at least one R^{V} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{V} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Va}.

In some embodiments, at least one R^{V} is oxazolyl.

In some embodiments, at least one R^{V} is

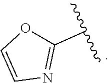 [SMILES: *c1ncco1]

In some embodiments, at least one R^{V} is thiazolyl.

In some embodiments, at least one R^{V} is

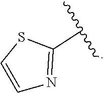 [SMILES: *c1nccs1]

In some embodiments, at least one R^{V} is pyrazolyl.

In some embodiments, at least one R^{V} is

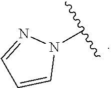 [SMILES: *n1cccn1]

In some embodiments, at least one R^{V} is

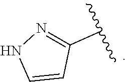 [SMILES: *c1cc[nH]n1]

In some embodiments, at least one R^{V} is isoxazolyl.

In some embodiments, at least one R^{V} is isothiazolyl.

In some embodiments, at least one R^{V} is imidazolyl.

In some embodiments, at least one R^{V} is 5-membered heteroaryl containing three heteroatoms selected from N, O, and S (e.g., triazolyl, oxadiazolyl, thiadiazolyl, or oxathiadiazolyl).

In some embodiments, at least one R^{V} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Va}.

In some embodiments, at least one R^{V} is triazolyl.

In some embodiments, at least one R^{V} is

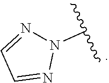 [SMILES: *n1nccn1]

In some embodiments, at least one R^{V} is

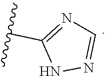 [SMILES: *c1ncn[nH]1]

In some embodiments, at least one R^{V} is

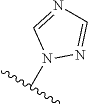 [SMILES: *n1cncn1]

In some embodiments, at least one R^{V} is oxadiazolyl.

In some embodiments, at least one R^{V} is

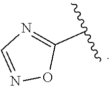 [SMILES: *c1ncno1]

In some embodiments, at least one R^{V} is thiadiazolyl.

In some embodiments, at least one R^{V} is

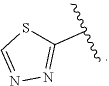 [SMILES: *c1nncs1]

In some embodiments, at least one R^{V} is

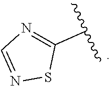 [SMILES: *c1ncns1]

In some embodiments, at least one R^{V} is 5-membered heteroaryl containing four heteroatoms selected from N, O, and S (e.g., tetrazolyl or oxatriazolyl).

In some embodiments, at least one R^{V} is 5-membered heteroaryl containing four heteroatoms selected from N, O, and S (e.g., tetrazolyl or oxatriazolyl), wherein the heteroaryl is substituted with one or more R^{Va}.

In some embodiments, at least one R^{V} is tetrazolyl.

In some embodiments, at least one R^{V} is

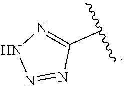 [SMILES: *c1nn[nH]n1]

In some embodiments, at least one R^{V} is oxatriazolyl.

In some embodiments, at least one R^{V} is 6-membered heteroaryl containing at least one heteroatom selected from N, O, and S.

In some embodiments, at least one R^{V} is 6-membered heteroaryl containing at least one heteroatom selected from N, O, and S, wherein the heteroaryl is substituted with one or more R^{Va}.

In some embodiments, at least one R^{V} is 6-membered heteroaryl containing one heteroatom selected from N, O, and S (e.g., pyridinyl, pyranyl, or thiopyranyl).

In some embodiments, at least one R^{V} is 6-membered heteroaryl containing one heteroatom selected from N, O, and S (e.g., pyridinyl, pyranyl, or thiopyranyl), wherein the heteroaryl is substituted with one or more R^{Va}.

In some embodiments, at least one R^{V} is pyridinyl.

In some embodiments, at least one R^{V} is

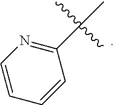 [SMILES: *c1ccccn1]

In some embodiments, at least one R^{V} is 6-membered heteroaryl containing two heteroatoms selected from N, O, and S (e.g., pyrimidinyl, pyridazinyl, or pyrazinyl).

In some embodiments, at least one R^{V} is 6-membered heteroaryl containing two heteroatoms selected from N, O, and S (e.g., pyrimidinyl, pyridazinyl, or pyrazinyl), wherein the heteroaryl is substituted with one or more R^{Va}.

In some embodiments, at least one R^{V} is pyrimidinyl.

In some embodiments, at least one R^{V} is

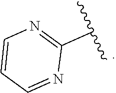 [SMILES: *c1ncccn1]

In some embodiments, at least one R^{V} is

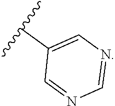 [SMILES: *c1cncnc1]

In some embodiments, at least one R^{V} is pyridazinyl.

In some embodiments, at least one R^{V} is

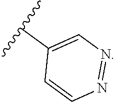 [SMILES: *c1ccnnc1]

In some embodiments, at least one R^{V} is pyrazinyl.

In some embodiments, at least one R^{V} is 9-membered heteroaryl containing at least one heteroatom selected from N, O, and S.

In some embodiments, at least one R^{V} is 9-membered heteroaryl containing at least one heteroatom selected from N, O, and S, wherein the heteroaryl is substituted with one or more R^{Va}.

In some embodiments, at least one R^{V} is

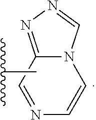

In some embodiments, at least one R^{V} is

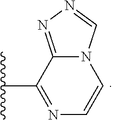 [SMILES: *c1nccn2cnnc12]

In some embodiments, at least one R^{V} is C₁-C₆ alkyl, C₂-C₆ alkenyl, C₂-C₆ alkynyl, cyclopropyl, cyclobutyl, cyclopentyl, cyclohexyl, cycloheptyl, cyclooctyl,

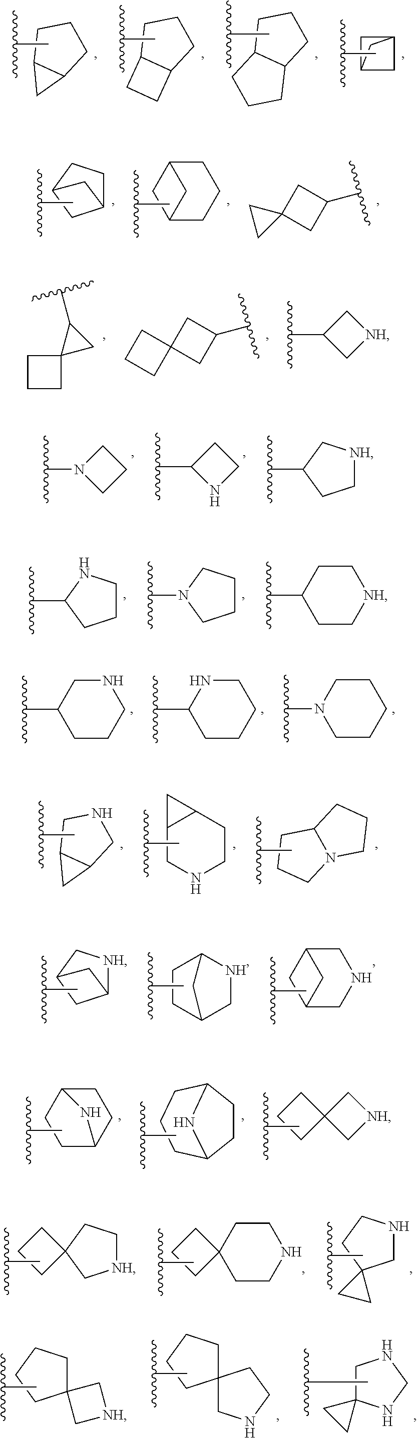

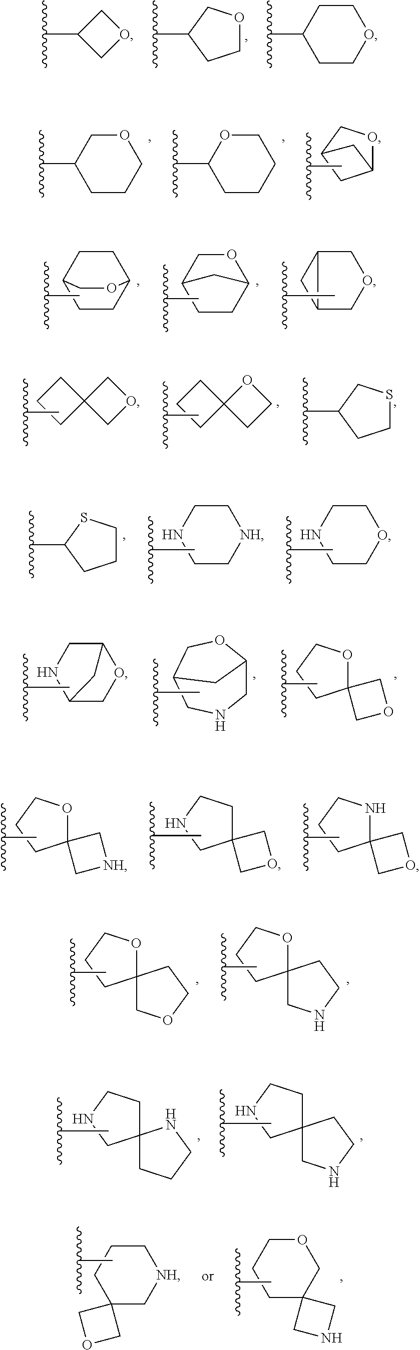

each optionally substituted with one or more R^{Va}.

In some embodiments, at least one R^{V} is pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, pyrazinyl, phenyl, or

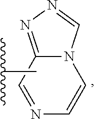

each optionally substituted with one or more R^{Va}.

### Variable R^{Va}

In some embodiments, at least one R^{Va} is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb}.

In some embodiments, at least one R^{Va} is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{Va} is oxo, halogen, cyano, —OH, —OR^{B}, —NH₂, —NHR^{B}, —N(R^{B})₂, —NH—C(═O)—H, —NH—C(═O)—R^{B}, —C(═O)—H, —C(═O)—R^{B}, —C(═O)—OH, —C(═O)—OR^{B}, —C(═O)—NH₂, —C(═O)—NHR^{B}, —C(═O)—N(R^{B})₂, —S(═O)₂—R^{B}, —NH—S(═O)₂—R^{B}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆cycloalkyl, 4- to 10-membered heterocyclyl, C₆-C₁₀ aryl, or 5- to 10-membered heteroaryl is substituted with one or more R^{Vb}.

In some embodiments, each R^{Va} independently is oxo, halogen, cyano, —OH, —OR^{B}, —NH₂, —NHR^{B}, —N(R^{B})₂, —C(═O)—H, —C(═O)—OH, —C(═O)—OR^{B}, —C(═O)—NH₂, —C(═O)—NHR^{B}, —C(═O)—N(R^{B})₂, C₁-C₆ alkyl, C₂-C₆ alkenyl, or C₂-C₆ alkynyl.

In some embodiments, at least one R^{Va} is oxo.

In some embodiments, at least one R^{Va} is halogen (e.g., —F, —Cl, —Br, —I).

In some embodiments, at least one R^{Va} is cyano.

In some embodiments, at least one R^{Va} is —OH.

In some embodiments, at least one R^{Va} is —OR^{B}.

In some embodiments, at least one R^{Va} is —NH₂.

In some embodiments, at least one R^{Va} is —NHR^{B}.

In some embodiments, at least one R^{Va} is —N(R^{B})₂.

In some embodiments, at least one R^{Va} is —NH—C(═O)—H.

In some embodiments, at least one R^{Va} is —NH—C(═O)—R^{B}.

In some embodiments, at least one R^{Va} is —C(═O)—H.

In some embodiments, at least one R^{Va} is —C(═O)—R^{B}.

In some embodiments, at least one R^{Va} is —C(═O)—OH.

In some embodiments, at least one R^{Va} is —C(═O)—OR^{B}.

In some embodiments, at least one R^{Va} is —C(═O)—NH₂.

In some embodiments, at least one R^{Va} is —C(═O)—NHR^{B}.

In some embodiments, at least one R^{Va} is —C(═O)—N(R^{B})₂.

In some embodiments, at least one R^{Va} is —S(═O)₂—R^{B}.

In some embodiments, at least one R^{Va} is —NH—S(═O)₂—R^{B}.

In some embodiments, at least one R^{Va} is C₁-C₆ alkyl (e.g., methyl, ethyl, n-propyl, isopropyl, n-butyl, t-butyl, isobutyl, or sec-butyl).

In some embodiments, at least one R^{Va} is C₁-C₆ alkyl (e.g., methyl, ethyl, n-propyl, isopropyl, n-butyl, t-butyl, isobutyl, or sec-butyl) substituted with one or more R^{Vb}.

In some embodiments, at least one R^{Va} is methyl.

In some embodiments, at least one R^{Va} is ethyl.

In some embodiments, at least one R^{Va} is n-propyl.

In some embodiments, at least one R^{Va} is isopropyl.

In some embodiments, at least one R^{Va} is n-butyl.

In some embodiments, at least one R^{Va} is C₂-C₆ alkenyl.

In some embodiments, at least one R^{Va} is C₂-C₆ alkenyl substituted with one or more R^{Vb}.

In some embodiments, at least one R^{Va} is C₂-C₆ alkynyl.

In some embodiments, at least one R^{Va} is C₂-C₆ alkynyl substituted with one or more R^{Vb}.

In some embodiments, at least one R^{Va} is C₃-C₆ cycloalkyl.

In some embodiments, at least one R^{Va} is C₃-C₆ cycloalkyl substituted with one or more R^{Vb}.

In some embodiments, at least one R^{Va} is C₃-C₆ monocyclic cycloalkyl.

In some embodiments, at least one R^{Va} is C₃-C₆ monocyclic cycloalkyl substituted with one or more R^{Vb}.

In some embodiments, at least one R^{Va} is cyclopropyl, cyclobutyl, cyclopentyl, cyclohexyl, cycloheptyl, or cyclooctyl.

In some embodiments, at least one R^{Va} is C₃-C₆ bicyclic cycloalkyl.

In some embodiments, at least one R^{Va} is C₃-C₆ bicyclic cycloalkyl substituted with one or more R^{Vb}.

In some embodiments, at least one R^{Va} is bicyclocyclobutyl, bicyclocyclopentyl, or bicyclocyclohexyl.

In some embodiments, at least one R^{Va} is C₄-C₆ fused bicyclic cycloalkyl.

In some embodiments, at least one R^{Va} is C₄-C₆ fused bicyclic cycloalkyl substituted with one or more R^{Vb}.

In some embodiments, at least one R^{Va} is

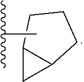

In some embodiments, at least one R^{Va} is

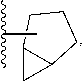

substituted with one or more R^{Vb}.

In some embodiments, at least one R^{Va} is

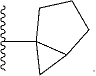 [SMILES: *C12CCCC1C2]

In some embodiments, at least one R^{Va} is

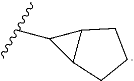 [SMILES: *C1C2CCCC12]

In some embodiments, at least one R^{Va} is C₅-C₆ bridged bicyclic cycloalkyl.

In some embodiments, at least one R^{Va} is C₅-C₆ bridged bicyclic cycloalkyl substituted with one or more R^{Vb}.

In some embodiments, at least one R^{Va} is

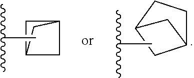

In some embodiments, at least one R^{Va} is

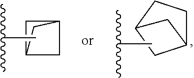

substituted with one or more R^{Vb}.

In some embodiments, at least one R^{Va} is

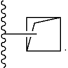

In some embodiments, at least one R^{Va} is

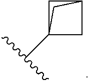 [SMILES: *C12CC(C1)C2]

In some embodiments, at least one R^{Va} is

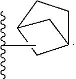

In some embodiments, at least one R^{Va} is

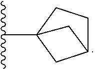 [SMILES: *C12CCC(C1)C2]

In some embodiments, at least one R^{Va} is C₅-C₆ spiro bicyclic cycloalkyl.

In some embodiments, at least one R^{Va} is C₅-C₆ spiro bicyclic cycloalkyl substituted with one or more R^{Vb}.

In some embodiments, at least one R^{Va} is spiro[2.2]pentyl or spiro[2.3]hexyl.

In some embodiments, at least one R^{Va} is spiro[2.2]pentyl or spiro[2.3]hexyl, substituted with one or more R^{Vb}.

In some embodiments, at least one R^{Va} is

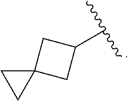 [SMILES: *C1CC2(CC2)C1]

In some embodiments, at least one R^{Va} is

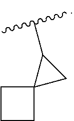 [SMILES: *C1CC12CCC2]

In some embodiments, at least one R^{Va} is 4- to 10-membered heterocyclyl.

In some embodiments, at least one R^{Va} is 4- to 10-membered heterocyclyl substituted with one or more R^{Vb}.

In some embodiments, at least one R^{Va} is 4- to 10-membered monocyclic heterocyclyl.

In some embodiments, at least one R^{Va} is 4- to 10-membered monocyclic heterocyclyl substituted with one or more R^{Vb}.

In some embodiments, at least one R^{Va} is 4- to 10-membered bicyclic heterocyclyl.

In some embodiments, at least one R^{Va} is 4- to 10-membered bicyclic heterocyclyl substituted with one or more R^{Vb}.

In some embodiments, at least one R^{Va} is 4- to 10-membered fused bicyclic heterocyclyl.

In some embodiments, at least one R^{Va} is 4- to 10-membered fused bicyclic heterocyclyl substituted with one or more R^{Vb}.

In some embodiments, at least one R^{Va} is 5- to 10-membered bridged bicyclic heterocyclyl.

In some embodiments, at least one R^{Va} is 5- to 10-membered bridged bicyclic heterocyclyl substituted with one or more R^{Vb}.

In some embodiments, at least one R^{Va} is 5- to 10-membered spiro bicyclic heterocyclyl.

In some embodiments, at least one R^{Va} is 5- to 10-membered spiro bicyclic heterocyclyl substituted with one or more R^{Vb}.

In some embodiments, at least one R^{Va} is 4- to 10-membered heterocyclyl containing at least one heteroatom selected from N, O, and S.

In some embodiments, at least one R^{Va} is 4- to 10-membered heterocyclyl containing at least one heteroatom selected from N, O, and S, wherein the 4- to 10-membered heterocyclyl is substituted with one or more R^{Vb}.

In some embodiments, at least one R^{Va} is 4- to 10-membered monocyclic heterocyclyl containing at least one heteroatom selected from N, O, and S.

In some embodiments, at least one R^{Va} is 4- to 10-membered monocyclic heterocyclyl containing at least one heteroatom selected from N, O, and S, wherein the 4- to 10-membered heterocyclyl is substituted with one or more R^{Vb}.

In some embodiments, at least one R^{Va} is 4- to 10-membered monocyclic heterocyclyl containing one N (e.g., azetidinyl, pyrrolidinyl, or piperidinyl).

In some embodiments, at least one R^{Va} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Vb}.

In some embodiments, at least one R^{Va} is azetidinyl.

In some embodiments, at least one R^{Va} is azetidinyl substituted with one or more R^{Vb}.

In some embodiments, at least one R^{Va} is

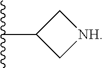 [SMILES: *C1CNC1]

In some embodiments, at least one R^{Va} is

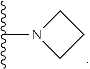 [SMILES: *N1CCC1]

In some embodiments, at least one R^{Va} is

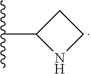 [SMILES: *C1CCN1]

In some embodiments, at least one R^{Va} is pyrrolidinyl.

In some embodiments, at least one R^{Va} is

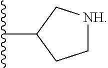 [SMILES: *C1CCNC1]

In some embodiments, at least one R^{Va} is

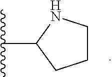 [SMILES: *C1CCCN1]

In some embodiments, at least one R^{Va} is

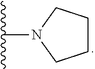 [SMILES: *N1CCCC1]

In some embodiments, at least one R^{Va} is piperidinyl.

In some embodiments, at least one R^{Va} is

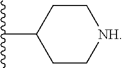 [SMILES: *C1CCNCC1]

In some embodiments, at least one R^{Va} is

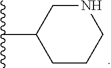 [SMILES: *C1CCCNC1]

In some embodiments, at least one R^{Va} is

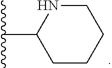 [SMILES: *C1CCCCN1]

In some embodiments, at least one R^{Va} is

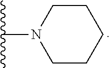 [SMILES: *N1CCCCC1]

In some embodiments, at least one R^{Va} is 4- to 10-membered fused bicyclic heterocyclyl containing one N.

In some embodiments, at least one R^{Va} is 4- to 10-membered fused bicyclic heterocyclyl containing one N, wherein the 4- to 10-membered fused bicyclic heterocyclyl is substituted with one or more R^{Vb}.

In some embodiments, at least one R^{Va} is

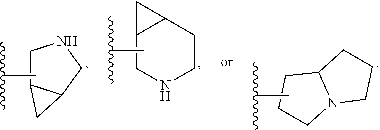

In some embodiments, at least one R^{Va} is

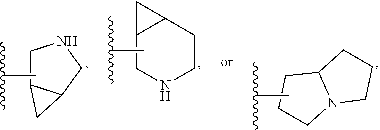

substituted with one or more R^{Vb}.

In some embodiments, at least one R^{Va} is

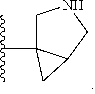 [SMILES: *C12CNCC1C2]

In some embodiments, at least one R^{Va} is

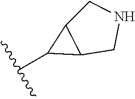 [SMILES: *C1C2CNCC12]

In some embodiments, at least one R^{Va} is

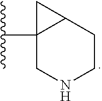 [SMILES: *C12CNCCC1C2]

In some embodiments, at least one R^{Va} is

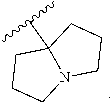 [SMILES: *C12CCCN1CCC2]

In some embodiments, at least one R^{Va} is 5- to 10-membered bridged bicyclic heterocyclyl containing one N.

In some embodiments, at least one R^{Va} is 5- to 10-membered bridged bicyclic heterocyclyl containing one N, wherein the 5- to 10-membered bridged bicyclic heterocyclyl is substituted with one or more R^{Vb}.

In some embodiments, at least one R^{Va} is

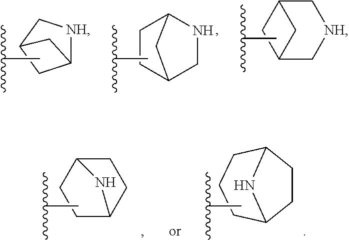

In some embodiments, at least one R^{Va} is

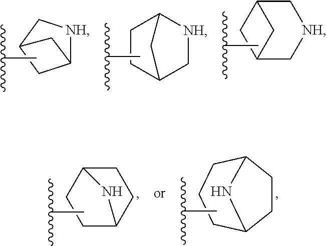

substituted with one or more R^{Vb}.

In some embodiments, at least one R^{Va} is

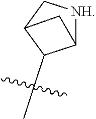 [SMILES: *C1C2CNC1C2]

In some embodiments, at least one R^{Va} is

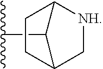 [SMILES: *C1C2CCC1NC2]

In some embodiments, at least one R^{Va} is

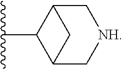 [SMILES: *C1C2CNCC1C2]

In some embodiments, at least one R^{Va} is

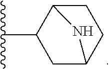 [SMILES: *C1CC2CCC1N2]

In some embodiments, at least one R^{Va} is

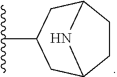 [SMILES: *C1CC2CCC(C1)N2]

In some embodiments, at least one R^{Va} is

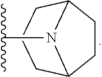 [SMILES: *N1C2CCCC1CC2]

In some embodiments, at least one R^{Va} is 5- to 10-membered spiro bicyclic heterocyclyl containing one N.

In some embodiments, at least one R^{Va} is 5- to 10-membered spiro bicyclic heterocyclyl containing one N, wherein the 5- to 10-membered spiro bicyclic heterocyclyl is substituted with one or more R^{Vb}.

In some embodiments, at least one R^{Va} is

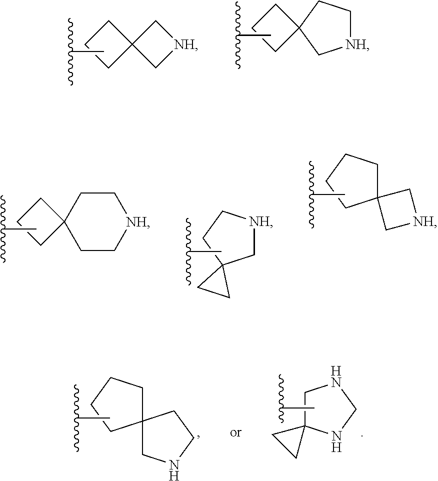

In some embodiments, at least one R^{Va} is

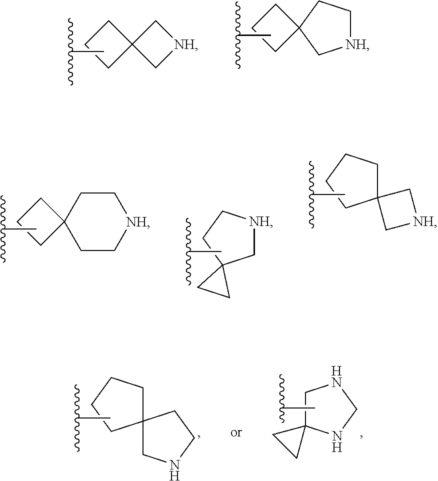

substituted with one or more R^{Vb}.

In some embodiments, at least one R^{Va} is

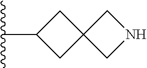 [SMILES: *C1CC2(CNC2)C1]

In some embodiments, at least one R^{Va} is

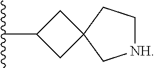 [SMILES: *C1CC2(CCNC2)C1]

In some embodiments, at least one R^{Va} is

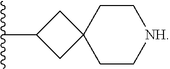 [SMILES: *C1CC2(CCNCC2)C1]

In some embodiments, at least one R^{Va} is

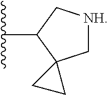 [SMILES: *C1CNCC12CC2]

In some embodiments, at least one R^{Va} is

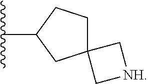 [SMILES: *C1CCC2(CNC2)C1]

In some embodiments, at least one R^{Va} is

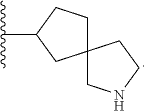 [SMILES: *C1CCC2(CCNC2)C1]

In some embodiments, at least one R^{Va} is 4- to 10-membered monocyclic heterocyclyl containing one O (e.g., oxetanyl, tetrahydrofuranyl, or tetrahydropyranyl).

In some embodiments, at least one R^{Va} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Vb}.

In some embodiments, at least one R^{Va} is oxetanyl.

In some embodiments, at least one R^{Va} is

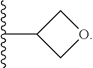 [SMILES: *C1COC1]

In some embodiments, at least one R^{Va} is

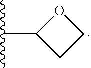 [SMILES: *C1CCO1]

In some embodiments, at least one R^{Va} is tetrahydrofuranyl.

In some embodiments, at least one R^{Va} is

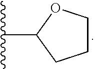 [SMILES: *C1CCCO1]

In some embodiments, at least one R^{Va} is

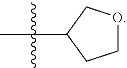 [SMILES: *C1CCOC1]

In some embodiments, at least one R^{Va} is tetrahydropyranyl.

In some embodiments, at least one R^{Va} is

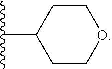 [SMILES: *C1CCOCC1]

In some embodiments, at least one R^{Va} is

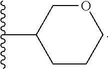 [SMILES: *C1CCCOC1]

In some embodiments, at least one R^{Va} is

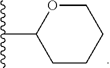 [SMILES: *C1CCCCO1]

In some embodiments, at least one R^{Va} is 5- to 10-membered bridged bicyclic heterocyclyl containing one O.

In some embodiments, at least one R^{Va} is 5- to 10-membered bridged bicyclic heterocyclyl containing one O, wherein the 5- to 10-membered bridged bicyclic heterocyclyl is substituted with one or more R^{Vb}.

In some embodiments, at least one R^{Va} is 4- to 10-membered fused bicyclic heterocyclyl containing one O.

In some embodiments, at least one R^{Va} is 4- to 10-membered fused bicyclic heterocyclyl containing one O, wherein the 4- to 10-membered fused heterocyclyl is substituted with one or more R^{Vb}.

In some embodiments, at least one R^{Va} is 5- to 10-membered spiro bicyclic heterocyclyl containing one O.

In some embodiments, at least one R^{Va} is 5- to 10-membered spiro bicyclic heterocyclyl containing one O, wherein the is 5- to 10-membered spiro bicyclic heterocyclyl is substituted with one or more R^{Vb}.

In some embodiments, at least one R^{Va} is

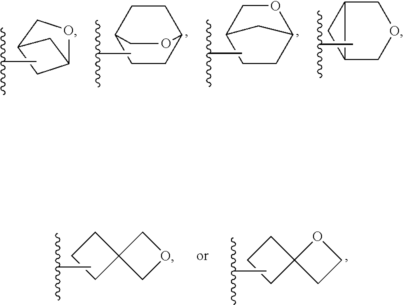

In some embodiments, at least one R^{Va} is P

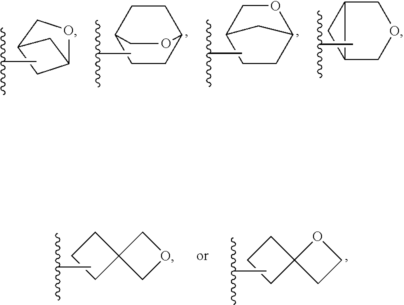

substituted with one or more R^{Vb}.

In some embodiments, at least one R^{Va} is

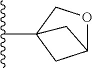 [SMILES: *C12COC(C1)C2]

In some embodiments, at least one R^{Va} is

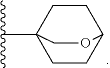 [SMILES: *C12CCC(CC1)OC2]

In some embodiments, at least one R^{Va} is

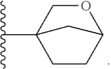 [SMILES: *C12CCC(C1)OC2]

In some embodiments, at least one R^{Va} is

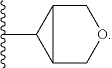 [SMILES: *C1C2COCC12]

In some embodiments, at least one R^{Va} is

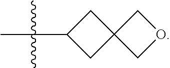 [SMILES: *C1CC2(COC2)C1]

In some embodiments, at least one R^{Va} is

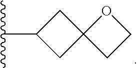 [SMILES: *C1CC2(CCO2)C1]

In some embodiments, at least one R^{Va} is 4- to 10-membered monocyclic heterocyclyl containing one S (e.g., tetrahydrothiophenyl).

In some embodiments, at least one R^{Va} is 4- to 10-membered monocyclic heterocyclyl containing one S (e.g., tetrahydrothiophenyl), wherein the 4- to 10-membered monocyclic heterocyclyl is substituted with one or more R^{Vb}.

In some embodiments, at least one R^{Va} is tetrahydrothiophenyl.

In some embodiments, at least one R^{Va} is

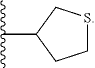 [SMILES: *C1CCSC1]

In some embodiments, at least one R^{Va} is

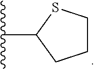 [SMILES: *C1CCCS1]

In some embodiments, at least one R^{Va} is 4- to 10-membered heterocyclyl (e.g., monocyclic, fused bicyclic, bridged bicyclic, or spiro bicyclic) containing two heteroatoms selected from N, O, and S.

In some embodiments, at least one R^{Va} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Vb}.

In some embodiments, at least one R^{Va} is 4- to 10-membered monocyclic heterocyclyl containing two heteroatoms selected from N, O, and S.

In some embodiments, at least one R^{Va} is 4- to 10-membered monocyclic heterocyclyl containing two heteroatoms selected from N, O, and S, wherein the 4- to 10-membered monocyclic heterocyclyl is substituted with one or more R^{Vb}.

In some embodiments, at least one R^{Va} is 4- to 10-membered fused bicyclic heterocyclyl containing two heteroatoms selected from N, O, and S.

In some embodiments, at least one R^{Va} is 4- to 10-membered fused bicyclic heterocyclyl containing two heteroatoms selected from N, O, and S, wherein the 4- to 10-membered fused bicyclic heterocyclyl is substituted with one or more R^{Vb}.

In some embodiments, at least one R^{Va} is 5- to 10-membered bridged bicyclic heterocyclyl containing two heteroatoms selected from N, O, and S.

In some embodiments, at least one R^{Va} is 5- to 10-membered bridged bicyclic heterocyclyl containing two heteroatoms selected from N, O, and S, wherein the 5- to 10-membered bridged bicyclic heterocyclyl is substituted with one or more R^{Vb}.

In some embodiments, at least one R^{Va} is 5- to 10-membered spiro bicyclic heterocyclyl containing two heteroatoms selected from N, O, and S.

In some embodiments, at least one R^{Va} is 5- to 10-membered spiro bicyclic heterocyclyl containing two heteroatoms selected from N, O, and S, wherein the 5- to 10-membered spiro bicyclic heterocyclyl is substituted with one or more R^{Vb}.

In some embodiments, at least one R^{Va} is

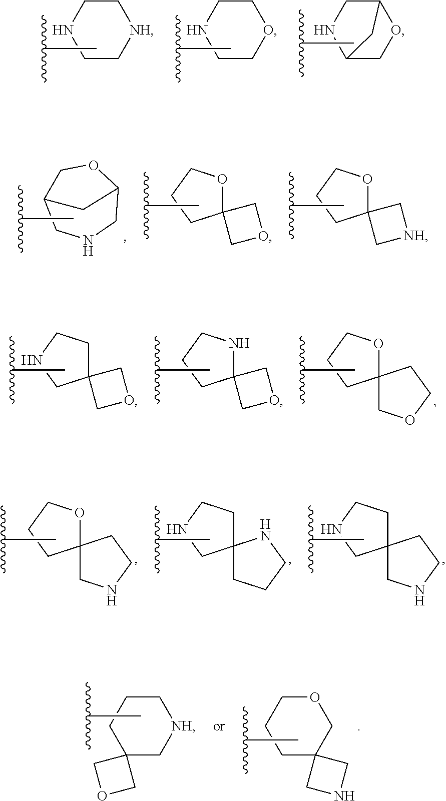

In some embodiments, at least one R^{Va} is

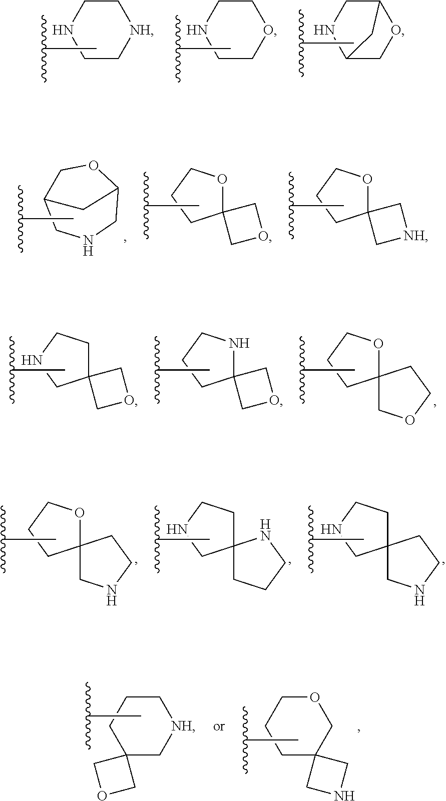

substituted with one or more R^{Vb}.

In some embodiments, at least one R^{Va} is

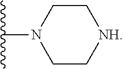 [SMILES: *N1CCNCC1]

In some embodiments, at least one R^{Va} is

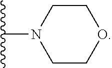 [SMILES: *N1CCOCC1]

In some embodiments, at least one R^{Va} is

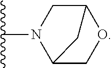 [SMILES: *N1CC2CC1CO2]

In some embodiments, at least one R^{Va} is

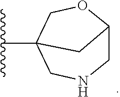 [SMILES: *C12CNCC(C1)OC2]

In some embodiments, at least one R^{Va} is

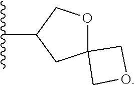 [SMILES: *C1COC2(COC2)C1]

In some embodiments, at least one R^{Va} is

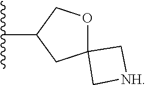 [SMILES: *C1COC2(CNC2)C1]

In some embodiments, at least one R^{Va} is

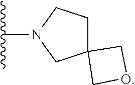 [SMILES: *N1CCC2(COC2)C1]

In some embodiments, at least one R^{Va} is

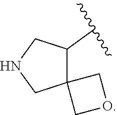 [SMILES: *C1CNCC12COC2]

In some embodiments, at least one R^{Va} is

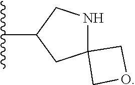 [SMILES: *C1CNC2(COC2)C1]

In some embodiments, at least one R^{Va} is

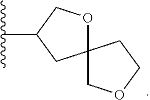 [SMILES: *C1COC2(CCOC2)C1]

In some embodiments, at least one R^{Va} is

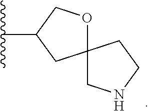 [SMILES: *C1COC2(CCNC2)C1]

In some embodiments, at least one R^{Va} is

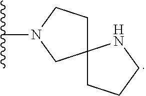 [SMILES: *N1CCC2(CCCN2)C1]

In some embodiments, at least one R^{Va} is

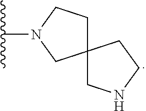 [SMILES: *N1CCC2(CCNC2)C1]

In some embodiments, at least one R^{Va} is

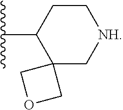 [SMILES: *C1CCNCC12COC2]

In some embodiments, at least one R^{Va} is

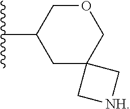 [SMILES: *C1COCC2(CNC2)C1]

In some embodiments, at least one R^{Va} is C₆-C₁₀ aryl (e.g., phenyl).

In some embodiments, at least one R^{Va} is C₆-C₁₀ aryl (e.g., phenyl) substituted with one or more R^{Vb}.

In some embodiments, at least one R^{Va} is phenyl.

In some embodiments, at least one R^{Va} is 5- to 10-membered heteroaryl.

In some embodiments, at least one R^{Va} is 5- to 10-membered heteroaryl substituted with one or more R^{Vb}.

In some embodiments, at least one R^{Va} is 5- to 10-membered heteroaryl containing at least one heteroatom selected from N, O, and S.

In some embodiments, at least one R^{Va} is 5- to 10-membered heteroaryl containing at least one heteroatom selected from N, O, and S, wherein the heteroaryl is substituted with one or more R^{Vb}.

In some embodiments, at least one R^{Va} is 5-membered heteroaryl containing at least one heteroatom selected from N, O, and S.

In some embodiments, at least one R^{Va} is 5-membered heteroaryl containing at least one heteroatom selected from N, O, and S, wherein the heteroaryl is substituted with one or more R^{Vb}.

In some embodiments, at least one R^{Va} is 5-membered heteroaryl containing one heteroatom selected from N, O, and S (e.g., pyrrolyl, furanyl, or thiophenyl).

In some embodiments, at least one R^{Va} is 5-membered heteroaryl containing one heteroatom selected from N, O, and S (e.g., pyrrolyl, furanyl, or thiophenyl), wherein the heteroaryl is substituted with one or more R^{Vb}.

In some embodiments, at least one R^{Va} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{Va} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Vb}.

In some embodiments, at least one R^{Va} is oxazolyl.

In some embodiments, at least one R^{Va} is

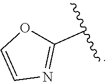 [SMILES: *c1ncco1]

In some embodiments, at least one R^{Va} is thiazolyl.

In some embodiments, at least one R^{Va} is

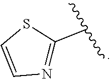 [SMILES: *c1nccs1]

In some embodiments, at least one R^{Va} is pyrazolyl.

In some embodiments, at least one R^{Va} is

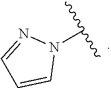 [SMILES: *n1cccn1]

In some embodiments, at least one R^{Va} is

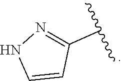 [SMILES: *c1cc[nH]n1]

In some embodiments, at least one R^{Va} is isoxazolyl.

In some embodiments, at least one R^{Va} is isothiazolyl.

In some embodiments, at least one R^{Va} is imidazolyl.

In some embodiments, at least one R^{Va} is 5-membered heteroaryl containing three heteroatoms selected from N, O, and S (e.g., triazolyl, oxadiazolyl, thiadiazolyl, or oxathiadiazolyl).

In some embodiments, at least one R^{Va} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Vb}.

In some embodiments, at least one R^{Va} is triazolyl.

In some embodiments, at least one R^{Va} is

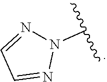 [SMILES: *n1nccn1]

In some embodiments, at least one R^{Va} is

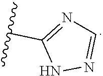 [SMILES: *c1ncn[nH]1]

In some embodiments, at least one R^{Va} is

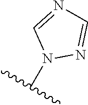 [SMILES: *n1cncn1]

In some embodiments, at least one R^{Va} is oxadiazolyl.

In some embodiments, at least one R^{Va} is

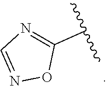 [SMILES: *c1ncno1]

In some embodiments, at least one R^{Va} is thiadiazolyl.

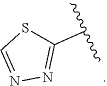 [SMILES: *c1nncs1]

In some embodiments, at least one R^{Va} is

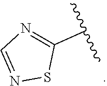 [SMILES: *c1ncns1]

In some embodiments, at least one R^{Va} is 5-membered heteroaryl containing four heteroatoms selected from N, O, and S (e.g., tetrazolyl or oxatriazolyl).

In some embodiments, at least one R^{Va} is 5-membered heteroaryl containing four heteroatoms selected from N, O, and S (e.g., tetrazolyl or oxatriazolyl), wherein the heteroaryl is substituted with one or more R^{Vb}.

In some embodiments, at least one R^{Va} is tetrazolyl.

In some embodiments, at least one R^{Va} is

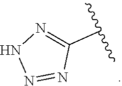 [SMILES: *c1nn[nH]n1]

In some embodiments, at least one R^{Va} is oxatriazolyl.

In some embodiments, at least one R^{Va} is 6-membered heteroaryl containing at least one heteroatom selected from N, O, and S.

In some embodiments, at least one R^{Va} is 6-membered heteroaryl containing at least one heteroatom selected from N, O, and S, wherein the heteroaryl is substituted with one or more R^{Vb}.

In some embodiments, at least one R^{Va} is 6-membered heteroaryl containing one heteroatom selected from N, O, and S (e.g., pyridinyl, pyranyl, or thiopyranyl).

In some embodiments, at least one R^{Va} is 6-membered heteroaryl containing one heteroatom selected from N, O, and S (e.g., pyridinyl, pyranyl, or thiopyranyl), wherein the heteroaryl is substituted with one or more R^{Vb}.

In some embodiments, at least one R^{Va} is pyridinyl.

In some embodiments, at least one R^{Va} is

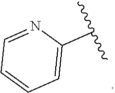 [SMILES: *c1ccccn1]

In some embodiments, at least one R^{Va} is 6-membered heteroaryl containing two heteroatoms selected from N, O, and S (e.g., pyrimidinyl, pyridazinyl, or pyrazinyl).

In some embodiments, at least one R^{Va} is 6-membered heteroaryl containing two heteroatoms selected from N, O, and S (e.g., pyrimidinyl, pyridazinyl, or pyrazinyl), wherein the heteroaryl is substituted with one or more R^{Vb}.

In some embodiments, at least one R^{Va} is pyrimidinyl.

In some embodiments, at least one R^{Va} is

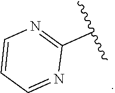 [SMILES: *c1ncccn1]

In some embodiments, at least one R^{Va} is

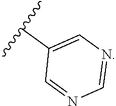 [SMILES: *c1cncnc1]

In some embodiments, at least one R^{Va} is pyridazinyl.

In some embodiments, at least one R^{Va} is

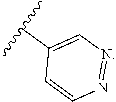 [SMILES: *c1ccnnc1]

In some embodiments, at least one R^{Va} is pyrazinyl.

In some embodiments, at least one R^{Va} is 9-membered heteroaryl containing at least one heteroatom selected from N, O, and S.

In some embodiments, at least one R^{Va} is 9-membered heteroaryl containing at least one heteroatom selected from N, O, and S, wherein the heteroaryl is substituted with one or more R^{Vb}.

In some embodiments, at least one R^{Va} is

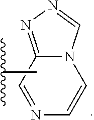

In some embodiments, at least one R^{Va} is

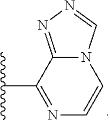 [SMILES: *c1nccn2cnnc12]

### Variable R^{Vb}

In some embodiments, at least one R^{Vb} is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc}.

In some embodiments, at least one R^{Vb} is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{Vb} is oxo, halogen, cyano, —OH, —OR^{C}, —NH₂, —NHR^{C}, —N(R^{C})₂, —NH—C(═O)—H, —NH—C(═O)—R^{C}, —C(═O)—H, —C(═O)—R^{C}, —C(═O)—OH, —C(═O)—OR^{C}, —C(═O)—NH₂, —C(═O)—NHR^{C}, —C(═O)—N(R^{C})₂, —S(═O)₂—R^{C}, —NH—S(═O)₂—R^{C}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆cycloalkyl, 4- to 10-membered heterocyclyl, C₆-C₁₀ aryl, or 5- to 10-membered heteroaryl is substituted with one or more R^{Vc}.

In some embodiments, at least one R^{Vb} is oxo, halogen, cyano, —OH, —OR^{c}, —NH₂, —NHR^{C}, —N(R^{C})₂, —C(═O)—H, —C(═O)—OH, —C(═O)—OR^{c}, —C(═O)—NH₂, —C(═O)—NHR^{C}, —C(═O)—N(R^{C})₂, C₁-C₆ alkyl, C₂-C₆ alkenyl, or C₂-C₆ alkynyl.

In some embodiments, at least one R^{Vb} is oxo.

In some embodiments, at least one R^{Vb} is halogen (e.g., —F, —Cl, —Br, —I).

In some embodiments, at least one R^{Vb} is cyano.

In some embodiments, at least one R^{Vb} is —OH.

In some embodiments, at least one R^{Vb} is —OR^{C}.

In some embodiments, at least one R^{Vb} is —NH₂.

In some embodiments, at least one R^{Vb} is —NHR^{C}.

In some embodiments, at least one R^{Vb} is —N(R^{C})₂.

In some embodiments, at least one R^{Vb} is —NH—C(═O)—H.

In some embodiments, at least one R^{Vb} is —NH—C(═O)—R^{C}.

In some embodiments, at least one R^{Vb} is —C(═O)—H.

In some embodiments, at least one R^{Vb} is —C(═O)—R^{C}.

In some embodiments, at least one R^{Vb} is —C(═O)—OH.

In some embodiments, at least one R^{Vb} is —C(═O)—OR^{C}.

In some embodiments, at least one R^{Vb} is —C(═O)—NH₂.

In some embodiments, at least one R^{Vb} is —C(═O)—NHR^{C}.

In some embodiments, at least one R^{Vb} is —C(═O)—N(R^{C})₂.

In some embodiments, at least one R^{Vb} is —S(═O)₂—R^{C}.

In some embodiments, at least one R^{Vb} is —NH—S(═O)₂—R^{C}.

In some embodiments, at least one R^{Vb} is C₁-C₆ alkyl (e.g., methyl, ethyl, n-propyl, isopropyl, n-butyl, t-butyl, isobutyl, or sec-butyl).

In some embodiments, at least one R^{Vb} is C₁-C₆ alkyl (e.g., methyl, ethyl, n-propyl, isopropyl, n-butyl, t-butyl, isobutyl, or sec-butyl) substituted with one or more R^{Vc}.

In some embodiments, at least one R^{Vb} is methyl.

In some embodiments, at least one R^{Vb} is ethyl.

In some embodiments, at least one R^{Vb} is n-propyl.

In some embodiments, at least one R^{Vb} is isopropyl.

In some embodiments, at least one R^{Vb} is n-butyl.

In some embodiments, at least one R^{Vb} is C₂-C₆ alkenyl.

In some embodiments, at least one R^{Vb} is C₂-C₆ alkenyl substituted with one or more R^{Vc}.

In some embodiments, at least one R^{Vb} is C₂-C₆ alkynyl.

In some embodiments, at least one R^{Vb} is C₂-C₆ alkynyl substituted with one or more R^{Vc}.

In some embodiments, at least one R^{Vb} is C₃-C₆ cycloalkyl.

In some embodiments, at least one R^{Vb} is C₃-C₆ cycloalkyl substituted with one or more R^{Vc}.

In some embodiments, at least one R^{Vb} is C₃-C₆ monocyclic cycloalkyl.

In some embodiments, at least one R^{Vb} is C₃-C₆ monocyclic cycloalkyl substituted with one or more R^{Vc}.

In some embodiments, at least one R^{Vb} is cyclopropyl, cyclobutyl, cyclopentyl, cyclohexyl, cycloheptyl, or cyclooctyl.

In some embodiments, at least one R^{Vb} is C₃-C₆ bicyclic cycloalkyl.

In some embodiments, at least one R^{Vb} is C₃-C₆ bicyclic cycloalkyl substituted with one or more R^{Vc}.

In some embodiments, at least one R^{Vb} is bicyclocyclobutyl, bicyclocyclopentyl, or bicyclocyclohexyl.

In some embodiments, at least one R^{Vb} is C₄-C₆ fused bicyclic cycloalkyl.

In some embodiments, at least one R^{Vb} is C₄-C₆ fused bicyclic cycloalkyl substituted with one or more R^{Vc}.

In some embodiments, at least one R^{Vb} is

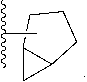

In some embodiments, at least one R^{Vb} is

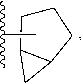

substituted with one or more R^{Vc}.

In some embodiments, at least one R^{Vb} is

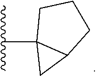 [SMILES: *C12CCCC1C2]

In some embodiments, at least one R^{Vb} is

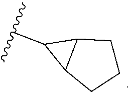 [SMILES: *C1C2CCCC12]

In some embodiments, at least one R^{Vb} is C₅-C₆ bridged bicyclic cycloalkyl.

In some embodiments, at least one R^{Vb} is C₅-C₆ bridged bicyclic cycloalkyl substituted with one or more R^{Vc}.

In some embodiments, at least one R^{Vb} is

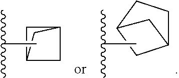

In some embodiments, at least one R^{Vb} is

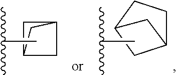

substituted with one or more R^{Vc}.

In some embodiments, at least one R^{Vb} is

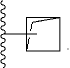

In some embodiments, at least one R^{Vb} is

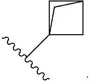 [SMILES: *C12CC(C1)C2]

In some embodiments, at least one R^{Vb} is

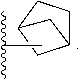

In some embodiments, at least one R^{Vb} is

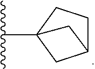 [SMILES: *C12CCC(C1)C2]

In some embodiments, at least one R^{Vb} is C₅-C₆ spiro bicyclic cycloalkyl.

In some embodiments, at least one R^{Vb} is C₅-C₆ spiro bicyclic cycloalkyl substituted with one or more R^{Vc}.

In some embodiments, at least one R^{Vb} is spiro[2.2]pentyl or spiro[2.3]hexyl.

In some embodiments, at least one R^{Vb} is spiro[2.2]pentyl or spiro[2.3]hexyl, substituted with one or more R^{Vc}.

In some embodiments, at least one R^{Vb} is

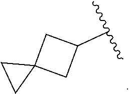 [SMILES: *C1CC2(CC2)C1]

In some embodiments, at least one R^{Vb} is

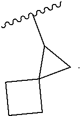 [SMILES: *C1CC12CCC2]

In some embodiments, at least one R^{Vb} is 4- to 10-membered heterocyclyl.

In some embodiments, at least one R^{Vb} is 4- to 10-membered heterocyclyl substituted with one or more R^{Vc}.

In some embodiments, at least one R^{Vb} is 4- to 10-membered monocyclic heterocyclyl.

In some embodiments, at least one R^{Vb} is 4- to 10-membered monocyclic heterocyclyl substituted with one or more R^{Vc}.

In some embodiments, at least one R^{Vb} is 4- to 10-membered bicyclic heterocyclyl.

In some embodiments, at least one R^{Vb} is 4- to 10-membered bicyclic heterocyclyl substituted with one or more R^{Vc}.

In some embodiments, at least one R^{Vb} is 4- to 10-membered fused bicyclic heterocyclyl.

In some embodiments, at least one R^{Vb} is 4- to 10-membered fused bicyclic heterocyclyl substituted with one or more R^{Vc}.

In some embodiments, at least one R^{Vb} is 5- to 10-membered bridged bicyclic heterocyclyl.

In some embodiments, at least one R^{Vb} is 5- to 10-membered bridged bicyclic heterocyclyl substituted with one or more R^{Vc}.

In some embodiments, at least one R^{Vb} is 5- to 10-membered spiro bicyclic heterocyclyl.

In some embodiments, at least one R^{Vb} is 5- to 10-membered spiro bicyclic heterocyclyl substituted with one or more R^{Vc}.

In some embodiments, at least one R^{Vb} is 4- to 10-membered heterocyclyl containing at least one heteroatom selected from N, O, and S.

In some embodiments, at least one R^{Vb} is 4- to 10-membered heterocyclyl containing at least one heteroatom selected from N, O, and S, wherein the 4- to 10-membered heterocyclyl is substituted with one or more R^{Vc}.

In some embodiments, at least one R^{Vb} is 4- to 10-membered monocyclic heterocyclyl containing at least one heteroatom selected from N, O, and S.

In some embodiments, at least one R^{Vb} is 4- to 10-membered monocyclic heterocyclyl containing at least one heteroatom selected from N, O, and S, wherein the 4- to 10-membered heterocyclyl is substituted with one or more R^{Vc}.

In some embodiments, at least one R^{Vb} is 4- to 10-membered monocyclic heterocyclyl containing one N (e.g., azetidinyl, pyrrolidinyl, or piperidinyl).

In some embodiments, at least one R^{Vb} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Vc}.

In some embodiments, at least one R^{Vb} is azetidinyl.

In some embodiments, at least one R^{Vb} is azetidinyl substituted with one or more R^{Vc}.

In some embodiments, at least one R^{Vb} is

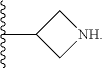 [SMILES: *C1CNC1]

In some embodiments, at least one R^{Vb} is

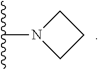 [SMILES: *N1CCC1]

In some embodiments, at least one R^{Vb} is

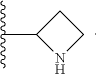 [SMILES: *C1CCN1]

In some embodiments, at least one R^{Vb} is pyrrolidinyl.

In some embodiments, at least one R^{Vb} is

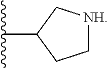 [SMILES: *C1CCNC1]

In some embodiments, at least one R^{Vb} is

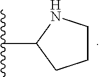 [SMILES: *C1CCCN1]

In some embodiments, at least one R^{Vb} is

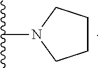 [SMILES: *N1CCCC1]

In some embodiments, at least one R^{Vb} is piperidinyl.

In some embodiments, at least one R^{Vb} is

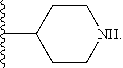 [SMILES: *C1CCNCC1]

In some embodiments, at least one R^{Vb} is

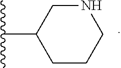 [SMILES: *C1CCCNC1]

In some embodiments, at least one R^{Vb} is

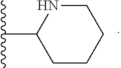 [SMILES: *C1CCCCN1]

In some embodiments, at least one R^{Vb} is

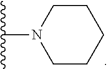 [SMILES: *N1CCCCC1]

In some embodiments, at least one R^{Vb} is 4- to 10-membered fused bicyclic heterocyclyl containing one N.

In some embodiments, at least one R^{Vb} is 4- to 10-membered fused bicyclic heterocyclyl containing one N, wherein the 4- to 10-membered fused bicyclic heterocyclyl is substituted with one or more R^{Vc}.

In some embodiments, at least one R^{Vb} is

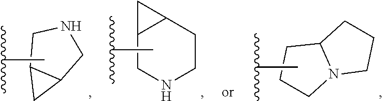

In some embodiments, at least one R^{Vb} is

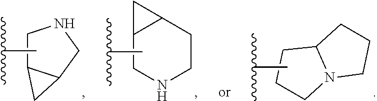

substituted with one or more R^{Vc}.

In some embodiments, at least one R^{Vb} is

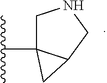 [SMILES: *C12CNCC1C2]

In some embodiments, at least one R^{Vb} is

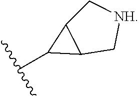 [SMILES: *C1C2CNCC12]

In some embodiments, at least one R^{Vb} is

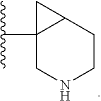 [SMILES: *C12CNCCC1C2]

In some embodiments, at least one R^{Vb} is

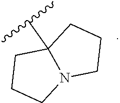 [SMILES: *C12CCCN1CCC2]

In some embodiments, at least one R^{Vb} is 5- to 10-membered bridged bicyclic heterocyclyl containing one N.

In some embodiments, at least one R^{Vb} is 5- to 10-membered bridged bicyclic heterocyclyl containing one N, wherein the 5- to 10-membered bridged bicyclic heterocyclyl is substituted with one or more R^{Vc}.

In some embodiments, at least one R^{Vb} is

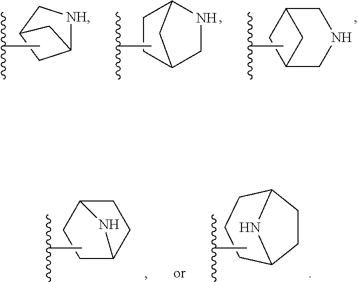

In some embodiments, at least one R^{Vb} is

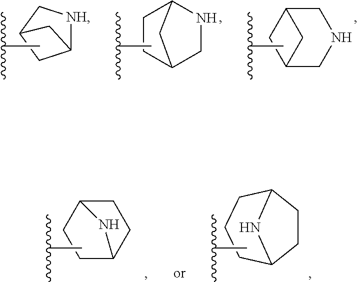

substituted with one or more R^{Vc}.

In some embodiments, at least one R^{Vb} is

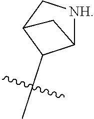 [SMILES: *C1C2CNC1C2]

In some embodiments, at least one R is

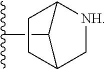 [SMILES: *C1C2CCC1NC2]

In some embodiments, at least one R^{Vb} is

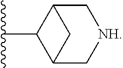 [SMILES: *C1C2CNCC1C2]

In some embodiments, at least one R^{Vb} is

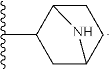 [SMILES: *C1CC2CCC1N2]

In some embodiments, at least one R^{Vb} is

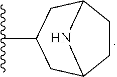 [SMILES: *C1CC2CCC(C1)N2]

In some embodiments, at least one R^{Vb} is

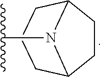 [SMILES: *N1C2CCCC1CC2]

In some embodiments, at least one R^{Vb} is 5- to 10-membered spiro bicyclic heterocyclyl containing one N.

In some embodiments, at least one R^{Vb} is 5- to 10-membered spiro bicyclic heterocyclyl containing one N, wherein the 5- to 10-membered spiro bicyclic heterocyclyl is substituted with one or more R^{Vc}.

In some embodiments, at least one R^{Vb} is

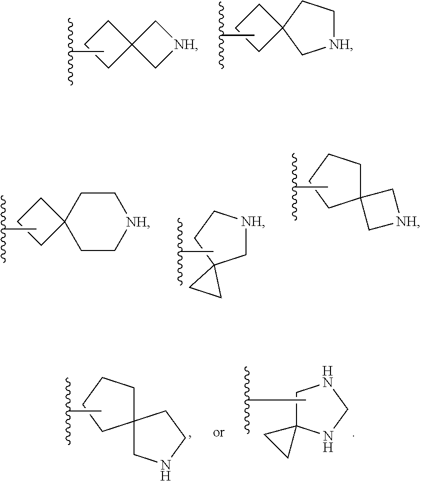

In some embodiments, at least one R^{Vb} is

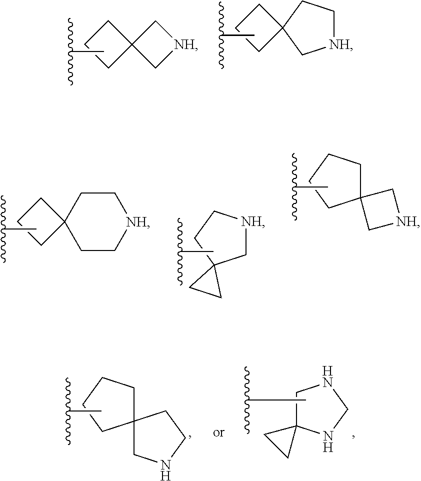

substituted with one or more R^{Vc}.

In some embodiments, at least one R^{Vb} is

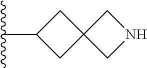 [SMILES: *C1CC2(CNC2)C1]

In some embodiments, at least one R^{Vb} is

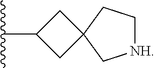 [SMILES: *C1CC2(CCNC2)C1]

In some embodiments, at least one R^{Vb} is

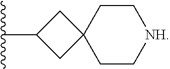 [SMILES: *C1CC2(CCNCC2)C1]

In some embodiments, at least one R^{Vb} is

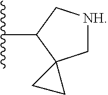 [SMILES: *C1CNCC12CC2]

In some embodiments, at least one R^{Vb} is

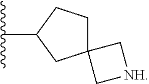 [SMILES: *C1CCC2(CNC2)C1]

In some embodiments, at least one R^{Vb} is

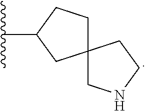 [SMILES: *C1CCC2(CCNC2)C1]

In some embodiments, at least one R^{Vb} is 4- to 10-membered monocyclic heterocyclyl containing one O (e.g., oxetanyl, tetrahydrofuranyl, or tetrahydropyranyl).

In some embodiments, at least one R^{Vb} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Vc}.

In some embodiments, at least one R^{Vb} is oxetanyl.

In some embodiments, at least one R^{Vb} is

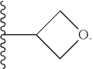 [SMILES: *C1COC1]

In some embodiments, at least one R^{Vb} is

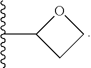 [SMILES: *C1CCO1]

In some embodiments, at least one R^{Vb} is tetrahydrofuranyl.

In some embodiments, at least one R^{Vb} is

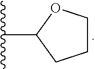 [SMILES: *C1CCCO1]

In some embodiments, at least one R^{Vb} is

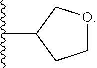 [SMILES: *C1CCOC1]

In some embodiments, at least one R^{Vb} is tetrahydropyranyl.

In some embodiments, at least one R^{Vb} is

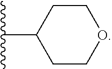 [SMILES: *C1CCOCC1]

In some embodiments, at least one R^{Vb} is

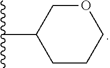 [SMILES: *C1CCCOC1]

In some embodiments, at least one R^{Vb} is

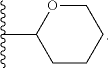 [SMILES: *C1CCCCO1]

In some embodiments, at least one R^{b} is 5- to 10-membered bridged bicyclic heterocyclyl containing one O.

In some embodiments, at least one R^{Vb} is 5- to 10-membered bridged bicyclic heterocyclyl containing one O, wherein the 5- to 10-membered bridged bicyclic heterocyclyl is substituted with one or more R^{Vc}.

In some embodiments, at least one R^{Vb} is 4- to 10-membered fused bicyclic heterocyclyl containing one O.

In some embodiments, at least one R^{Vb} is 4- to 10-membered fused bicyclic heterocyclyl containing one O, wherein the 4- to 10-membered fused heterocyclyl is substituted with one or more R^{Vc}.

In some embodiments, at least one R^{Vb} is 5- to 10-membered spiro bicyclic heterocyclyl containing one O.

In some embodiments, at least one R^{Vb} is 5- to 10-membered spiro bicyclic heterocyclyl containing one O, wherein the is 5- to 10-membered spiro bicyclic heterocyclyl is substituted with one or more R^{Vc}.

In some embodiments, at least one R^{Vb} is

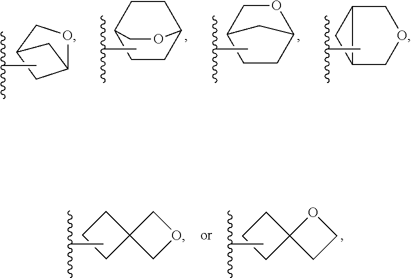

In some embodiments, at least one R^{Vb} is

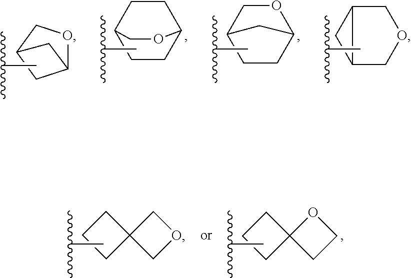

substituted with one or more R^{Vc}.

In some embodiments, at least one R^{Vb} is

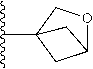 [SMILES: *C12COC(C1)C2]

In some embodiments, at least one R^{Vb} is

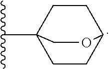 [SMILES: *C12CCC(CC1)OC2]

In some embodiments, at least one R^{Vb} is

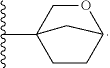 [SMILES: *C12CCC(C1)OC2]

In some embodiments, at least one R^{Vb} is

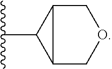 [SMILES: *C1C2COCC12]

In some embodiments, at least one R^{Vb} is

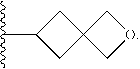 [SMILES: *C1CC2(COC2)C1]

In some embodiments, at least one R^{Vb} is

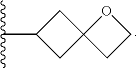 [SMILES: *C1CC2(CCO2)C1]

In some embodiments, at least one R^{Vb} is 4- to 10-membered monocyclic heterocyclyl containing one S (e.g., tetrahydrothiophenyl).

In some embodiments, at least one R^{Vb} is 4- to 10-membered monocyclic heterocyclyl containing one S (e.g., tetrahydrothiophenyl), wherein the 4- to 10-membered monocyclic heterocyclyl is substituted with one or more R^{Vc}.

In some embodiments, at least one R^{Vb} is tetrahydrothiophenyl.

In some embodiments, at least one R^{Vb} is

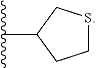 [SMILES: *C1CCSC1]

In some embodiments, at least one R^{Vb} is

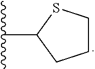 [SMILES: *C1CCCS1]

In some embodiments, at least one R^{Vb} is 4- to 10-membered heterocyclyl (e.g., monocyclic, fused bicyclic, bridged bicyclic, or spiro bicyclic) containing two heteroatoms selected from N, O, and S.

In some embodiments, at least one R^{Vb} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Vc}.

In some embodiments, at least one R^{Vb} is 4- to 10-membered monocyclic heterocyclyl containing two heteroatoms selected from N, O, and S.

In some embodiments, at least one R^{Vb} is 4- to 10-membered monocyclic heterocyclyl containing two heteroatoms selected from N, O, and S, wherein the 4- to 10-membered monocyclic heterocyclyl is substituted with one or more R^{Vc}.

In some embodiments, at least one R^{Vb} is 4- to 10-membered fused bicyclic heterocyclyl containing two heteroatoms selected from N, O, and S.

In some embodiments, at least one R^{Vb} is 4- to 10-membered fused bicyclic heterocyclyl containing two heteroatoms selected from N, O, and S, wherein the 4- to 10-membered fused bicyclic heterocyclyl is substituted with one or more R^{Vc}.

In some embodiments, at least one R^{Vb} is 5- to 10-membered bridged bicyclic heterocyclyl containing two heteroatoms selected from N, O, and S.

In some embodiments, at least one R^{Vb} is 5- to 10-membered bridged bicyclic heterocyclyl containing two heteroatoms selected from N, O, and S, wherein the 5- to 10-membered bridged bicyclic heterocyclyl is substituted with one or more R^{Vc}.

In some embodiments, at least one R^{Vb} is 5- to 10-membered spiro bicyclic heterocyclyl containing two heteroatoms selected from N, O, and S.

In some embodiments, at least one R^{Vb} is 5- to 10-membered spiro bicyclic heterocyclyl containing two heteroatoms selected from N, O, and S, wherein the 5- to 10-membered spiro bicyclic heterocyclyl is substituted with one or more R^{Vc}.

In some embodiments, at least one R^{Vb} is

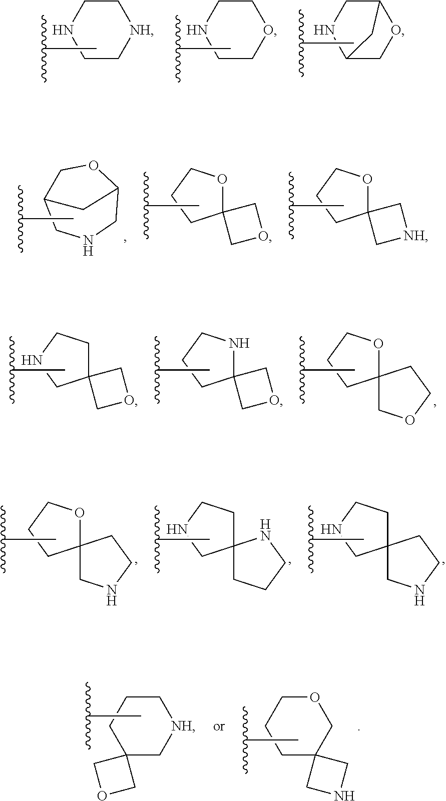

In some embodiments, at least one R^{Vb} is

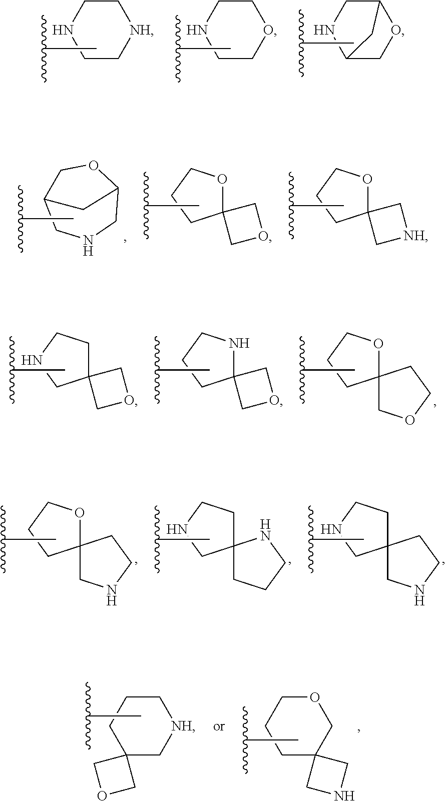

substituted with one or more R^{Vc}.

In some embodiments, at least one R^{Vb} is

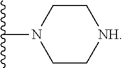 [SMILES: *N1CCNCC1]

In some embodiments, at least one R^{Vb} is

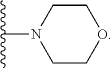 [SMILES: *N1CCOCC1]

In some embodiments, at least one R^{Vb} is

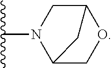 [SMILES: *N1CC2CC1CO2]

In some embodiments, at least one R^{Vb} is

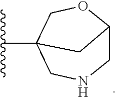 [SMILES: *C12CNCC(C1)OC2]

In some embodiments, at least one R^{Vb} is

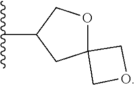 [SMILES: *C1COC2(COC2)C1]

In some embodiments, at least one R^{Vb} is

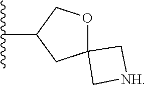 [SMILES: *C1COC2(CNC2)C1]

In some embodiments, at least one R^{Vb} is

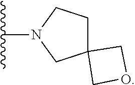 [SMILES: *N1CCC2(COC2)C1]

In some embodiments, at least one R^{Vb} is

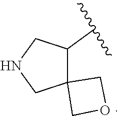 [SMILES: *C1CNCC12COC2]

In some embodiments, at least one R^{Vb} is

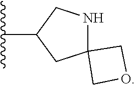 [SMILES: *C1CNC2(COC2)C1]

In some embodiments, at least one R^{Vb} is

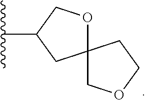 [SMILES: *C1COC2(CCOC2)C1]

In some embodiments, at least one R^{Vb} is

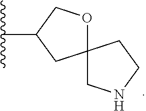 [SMILES: *C1COC2(CCNC2)C1]

In some embodiments, at least one R^{Vb} is

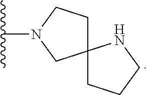 [SMILES: *N1CCC2(CCCN2)C1]

In some embodiments, at least one R^{Vb} is H

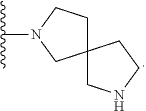 [SMILES: *N1CCC2(CCNC2)C1]

In some embodiments, at least one R^{Vb} is

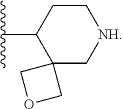 [SMILES: *C1CCNCC12COC2]

In some embodiments, at least one R^{Vb} is

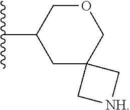 [SMILES: *C1COCC2(CNC2)C1]

In some embodiments, at least one R^{Vb} is C₆-C₁₀ aryl (e.g., phenyl).

In some embodiments, at least one R^{Vb} is C₆-C₁₀ aryl (e.g., phenyl) substituted with one or more R^{Vc}.

In some embodiments, at least one R^{Vb} is phenyl.

In some embodiments, at least one R^{Vb} is 5- to 10-membered heteroaryl.

In some embodiments, at least one R^{Vb} is 5- to 10-membered heteroaryl substituted with one or more R^{Vc}.

In some embodiments, at least one R^{Vb} is 5- to 10-membered heteroaryl containing at least one heteroatom selected from N, O, and S.

In some embodiments, at least one R^{Vb} is 5- to 10-membered heteroaryl containing at least one heteroatom selected from N, O, and S, wherein the heteroaryl is substituted with one or more R^{Vc}.

In some embodiments, at least one R^{Vb} is 5-membered heteroaryl containing at least one heteroatom selected from N, O, and S.

In some embodiments, at least one R^{Vb} is 5-membered heteroaryl containing at least one heteroatom selected from N, O, and S, wherein the heteroaryl is substituted with one or more R^{Vc}.

In some embodiments, at least one R^{Vb} is 5-membered heteroaryl containing one heteroatom selected from N, O, and S (e.g., pyrrolyl, furanyl, or thiophenyl).

In some embodiments, at least one R^{Vb} is 5-membered heteroaryl containing one heteroatom selected from N, O, and S (e.g., pyrrolyl, furanyl, or thiophenyl), wherein the heteroaryl is substituted with one or more R^{Vc}.

In some embodiments, at least one R^{Vb} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{Vb} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Vc}.

In some embodiments, at least one R^{Vb} is oxazolyl.

In some embodiments, at least one R^{Vb} is

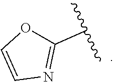 [SMILES: *c1ncco1]

In some embodiments, at least one R^{Vb} is thiazolyl.

In some embodiments, at least one R^{Vb} is

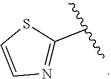 [SMILES: *c1nccs1]

In some embodiments, at least one R^{Vb} is pyrazolyl.

In some embodiments, at least one R^{Vb} is

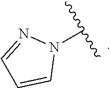 [SMILES: *n1cccn1]

In some embodiments, at least one R^{Vb} is

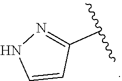 [SMILES: *c1cc[nH]n1]

In some embodiments, at least one R^{Vb} is isoxazolyl.

In some embodiments, at least one R^{Vb} is isothiazolyl.

In some embodiments, at least one R^{Vb} is imidazolyl.

In some embodiments, at least one R^{Vb} is 5-membered heteroaryl containing three heteroatoms selected from N, O, and S (e.g., triazolyl, oxadiazolyl, thiadiazolyl, or oxathiadiazolyl).

In some embodiments, at least one R^{Vb} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Vc}.

In some embodiments, at least one R^{Vb} is triazolyl.

In some embodiments, at least one R^{Vb} is

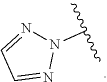 [SMILES: *n1nccn1]

In some embodiments, at least one R^{Vb} is

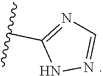 [SMILES: *c1ncn[nH]1]

In some embodiments, at least one R^{Vb} is

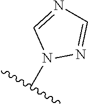 [SMILES: *n1cncn1]

In some embodiments, at least one R^{Vb} is oxadiazolyl.

In some embodiments, at least one R^{Vb} is

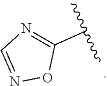 [SMILES: *c1ncno1]

In some embodiments, at least one R^{Vb} is thiadiazolyl.

In some embodiments, at least one R^{Vb} is

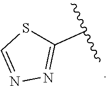 [SMILES: *c1nncs1]

In some embodiments, at least one R^{Vb} is

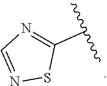 [SMILES: *c1ncns1]

In some embodiments, at least one R^{Vb} is 5-membered heteroaryl containing four heteroatoms selected from N, O, and S (e.g., tetrazolyl or oxatriazolyl).

In some embodiments, at least one R^{Vb} is 5-membered heteroaryl containing four heteroatoms selected from N, O, and S (e.g., tetrazolyl or oxatriazolyl), wherein the heteroaryl is substituted with one or more R^{Vc}.

In some embodiments, at least one R^{Vb} is tetrazolyl.

In some embodiments, at least one R^{Vb} is

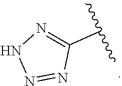 [SMILES: *c1nn[nH]n1]

In some embodiments, at least one R^{Vb} is oxatriazolyl.

In some embodiments, at least one R^{Vb} is 6-membered heteroaryl containing at least one heteroatom selected from N, O, and S.

In some embodiments, at least one R^{Vb} is 6-membered heteroaryl containing at least one heteroatom selected from N, O, and S, wherein the heteroaryl is substituted with one or more R^{Vc}.

In some embodiments, at least one R^{Vb} is 6-membered heteroaryl containing one heteroatom selected from N, O, and S (e.g., pyridinyl, pyranyl, or thiopyranyl).

In some embodiments, at least one R^{Vb} is 6-membered heteroaryl containing one heteroatom selected from N, O, and S (e.g., pyridinyl, pyranyl, or thiopyranyl), wherein the heteroaryl is substituted with one or more R^{Vc}.

In some embodiments, at least one R^{Vb} is pyridinyl.

In some embodiments, at least one R^{Vb} is

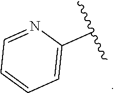 [SMILES: *c1ccccn1]

In some embodiments, at least one R^{Vb} is 6-membered heteroaryl containing two heteroatoms selected from N, O, and S (e.g., pyrimidinyl, pyridazinyl, or pyrazinyl).

In some embodiments, at least one R^{Vb} is 6-membered heteroaryl containing two heteroatoms selected from N, O, and S (e.g., pyrimidinyl, pyridazinyl, or pyrazinyl), wherein the heteroaryl is substituted with one or more R^{Vc}.

In some embodiments, at least one R^{Vb} is pyrimidinyl.

In some embodiments, at least one R^{Vb} is

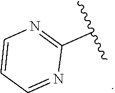 [SMILES: *c1ncccn1]

In some embodiments, at least one R^{Vb} is

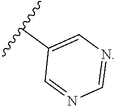 [SMILES: *c1cncnc1]

In some embodiments, at least one R^{Vb} is pyridazinyl.

In some embodiments, at least one R^{Vb} is

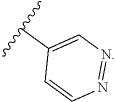 [SMILES: *c1ccnnc1]

In some embodiments, at least one R^{Vb} is pyrazinyl.

In some embodiments, at least one R^{Vb} is 9-membered heteroaryl containing at least one heteroatom selected from N, O, and S.

In some embodiments, at least one R^{Vb} is 9-membered heteroaryl containing at least one heteroatom selected from N, O, and S, wherein the heteroaryl is substituted with one or more R^{Vc}.

In some embodiments, at least one R^{Vb} is N

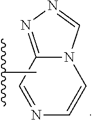

In some embodiments, at least one R^{Vb} is

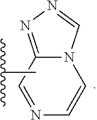

### Variable R^{Vc}

In some embodiments, at least one R^{Vc} is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

In some embodiments, at least one R^{Vc} is oxo.

In some embodiments, at least one R^{Vc} is halogen (e.g., —F, —Cl, —Br, —I).

In some embodiments, at least one R^{Vc} is cyano.

In some embodiments, at least one R^{Vc} is —OH.

In some embodiments, at least one R^{Vc} is —O(C₁-C₆ alkyl) (e.g., —O(methyl), —O(ethyl), —O(n-propyl), —O(isopropyl), —O(n-butyl), —O(t-butyl), —O(isobutyl), or —O(sec-butyl)).

In some embodiments, at least one R^{Vc} is —NH₂.

In some embodiments, at least one R^{Vc} is —NH(C₁-C₆ alkyl) (e.g., —NHCH₃, —NHCH₂CH₃, —NH(CH₂)₂CH₃, —NH(CH₂)₃CH₃, —NH(CH₂)₄CH₃, or —NH(CH₂)₅CH₃).

In some embodiments, at least one R^{Vc} is —N(C₁-C₆ alkyl)₂ (e.g., e.g., —N(methyl)₂, —N(ethyl)₂, —N(n-propyl)₂, —N(isopropyl)₂, —N(n-butyl)₂, —N(t-butyl)₂, —N(isobutyl)₂, or —N(sec-butyl)₂).

In some embodiments, at least one R^{Vc} is —C(═O)—H.

In some embodiments, at least one R^{Vc} is —C(═O)—OH.

In some embodiments, at least one R^{Vc} is —C(═O)—O(C₁-C₆ alkyl) (e.g., is —C(═O)—OCH₃, —C(═O)—OCH₂CH₃, —C(═O)—O(CH₂)₂CH₃, —C(═O)—O(CH₂)₃CH₃, —C(═O)—O(CH₂)₄CH₃, or —C(═O)—O(CH₂)₅CH₃).

In some embodiments, at least one R^{Vc} is —C(═O)—NH₂.

In some embodiments, at least one R^{Vc} is —C(═O)—NH(C₁-C₆ alkyl) ((e.g., —C(═O)—NHCH₃, —C(═O)—NHCH₂CH₃, —C(═O)—NH(CH₂)₂CH₃, —C(═O)—NH(CH₂)₃CH₃, —C(═O)—NH(CH₂)₄CH₃, or —C(═O)—NH(CH₂)₅CH₃)).

In some embodiments, at least one R^{Vc} is —C(═O)—N(C₁-C₆ alkyl)₂ (e.g., —C(═O)—N(methyl)₂, —C(═O)—N(ethyl)₂, —C(═O)—N(n-propyl)₂, —C(═O)—N(isopropyl)₂, —C(═O)—N(n-butyl)₂, —C(═O)—N(t-butyl)₂, —C(═O)—N(isobutyl)₂, or —C(═O)—N(sec-butyl)₂).

In some embodiments, at least one R^{Vc} is C₁-C₆ alkyl (e.g., methyl, ethyl, n-propyl, isopropyl, n-butyl, t-butyl, isobutyl, or sec-butyl).

In some embodiments, at least one R^{Vc} is methyl.

In some embodiments, at least one R^{Vc} is ethyl.

In some embodiments, at least one R^{Vc} is n-propyl.

In some embodiments, at least one R^{Vc} is isopropyl.

In some embodiments, at least one R^{Vc} is n-butyl.

In some embodiments, at least one R^{Vc} is C₂-C₆ alkenyl.

In some embodiments, at least one R^{Vc} is C₂-C₆ alkynyl.

### Variable R^{A}

In some embodiments, at least one R^{A} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vb}.

In some embodiments, at least one R^{A} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{A} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{Vb}.

In some embodiments, at least one R^{A} is C₁-C₆ alkyl (e.g., methyl, ethyl, n-propyl, isopropyl, n-butyl, t-butyl, isobutyl, or sec-butyl).

In some embodiments, at least one R^{A} is C₁-C₆ alkyl (e.g., methyl, ethyl, n-propyl, isopropyl, n-butyl, t-butyl, isobutyl, or sec-butyl) substituted with one or more R^{Vb}.

In some embodiments, at least one R^{A} is methyl.

In some embodiments, at least one R^{A} is ethyl.

In some embodiments, at least one R^{A} is n-propyl.

In some embodiments, at least one R^{A} is isopropyl.

In some embodiments, at least one R^{A} is n-butyl.

In some embodiments, at least one R^{A} is C₂-C₆ alkenyl.

In some embodiments, at least one R^{A} is C₂-C₆ alkenyl substituted with one or more R^{Vb}.

In some embodiments, at least one R^{A} is C₂-C₆ alkynyl.

In some embodiments, at least one R^{A} is C₂-C₆ alkynyl substituted with one or more R^{Vb}.

In some embodiments, at least one R^{A} is C₃-C₆ cycloalkyl.

In some embodiments, at least one R^{A} is C₃-C₆ cycloalkyl substituted with one or more R^{Vb}.

In some embodiments, at least one R^{A} is C₃-C₆ monocyclic cycloalkyl.

In some embodiments, at least one R^{A} is C₃-C₆ monocyclic cycloalkyl substituted with one or more R^{Vb}.

In some embodiments, at least one R^{A} is cyclopropyl, cyclobutyl, cyclopentyl, cyclohexyl, cycloheptyl, or cyclooctyl.

In some embodiments, at least one R^{A} is C₃-C₆ bicyclic cycloalkyl.

In some embodiments, at least one R^{A} is C₃-C₆ bicyclic cycloalkyl substituted with one or more R^{Vb}.

In some embodiments, at least one R^{A} is bicyclocyclobutyl, bicyclocyclopentyl, or bicyclocyclohexyl.

In some embodiments, at least one R^{A} is C₄-C₆ fused bicyclic cycloalkyl.

In some embodiments, at least one R^{A} is C₄-C₆ fused bicyclic cycloalkyl substituted with one or more R^{Vb}.

In some embodiments, at least one R^{A} is

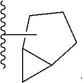

In some embodiments, at least one R^{A} is

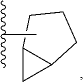

substituted with one or more R^{Vb}.

In some embodiments, at least one R^{A} is

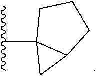 [SMILES: *C12CCCC1C2]

In some embodiments, at least one R^{A} is

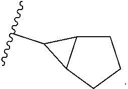 [SMILES: *C1C2CCCC12]

In some embodiments, at least one R^{A} is C₅-C₆ bridged bicyclic cycloalkyl.

In some embodiments, at least one R^{A} is C₅-C₆ bridged bicyclic cycloalkyl substituted with one or more R^{Vb}.

In some embodiments, at least one R^{A} is

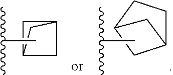

In some embodiments, at least one R^{A} is

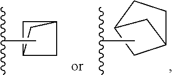

substituted with one or more R^{Vb}.

In some embodiments, at least one R^{A} is

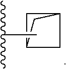

In some embodiments, at least one R^{A} is

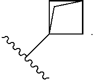 [SMILES: *C12CC(C1)C2]

In some embodiments, at least one R^{A} is

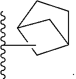

In some embodiments, at least one R^{A} is

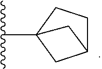 [SMILES: *C12CCC(C1)C2]

In some embodiments, at least one R^{A} is C₅-C₆ spiro bicyclic cycloalkyl.

In some embodiments, at least one R^{A} is C₅-C₆ spiro bicyclic cycloalkyl substituted with one or more R^{Vb}.

In some embodiments, at least one R^{A} is spiro[2.2]pentyl or spiro[2.3]hexyl.

In some embodiments, at least one R^{A} is spiro[2.2]pentyl or spiro[2.3]hexyl, substituted with one or more R^{Vb}.

In some embodiments, at least one R^{A} is

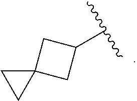 [SMILES: *C1CC2(CC2)C1]

In some embodiments, at least one R^{A} is

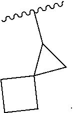 [SMILES: *C1CC12CCC2]

In some embodiments, at least one R^{A} is 4- to 10-membered heterocyclyl.

In some embodiments, at least one R^{A} is 4- to 10-membered heterocyclyl substituted with one or more R^{Vb}.

In some embodiments, at least one R^{A} is 4- to 10-membered monocyclic heterocyclyl.

In some embodiments, at least one R^{A} is 4- to 10-membered monocyclic heterocyclyl substituted with one or more R^{Vb}.

In some embodiments, at least one R^{A} is 4- to 10-membered bicyclic heterocyclyl.

In some embodiments, at least one R^{A} is 4- to 10-membered bicyclic heterocyclyl substituted with one or more R^{Vb}.

In some embodiments, at least one R^{A} is 4- to 10-membered fused bicyclic heterocyclyl.

In some embodiments, at least one R^{A} is 4- to 10-membered fused bicyclic heterocyclyl substituted with one or more R^{Vb}.

In some embodiments, at least one R^{A} is 5- to 10-membered bridged bicyclic heterocyclyl.

In some embodiments, at least one R^{A} is 5- to 10-membered bridged bicyclic heterocyclyl substituted with one or more R^{Vb}.

In some embodiments, at least one R^{A} is 5- to 10-membered spiro bicyclic heterocyclyl.

In some embodiments, at least one R^{A} is 5- to 10-membered spiro bicyclic heterocyclyl substituted with one or more R^{Vb}.

In some embodiments, at least one R^{A} is 4- to 10-membered heterocyclyl containing at least one heteroatom selected from N, O, and S.

In some embodiments, at least one R^{A} is 4- to 10-membered heterocyclyl containing at least one heteroatom selected from N, O, and S, wherein the 4- to 10-membered heterocyclyl is substituted with one or more R^{Vb}.

In some embodiments, at least one R^{A} is 4- to 10-membered monocyclic heterocyclyl containing at least one heteroatom selected from N, O, and S.

In some embodiments, at least one R^{A} is 4- to 10-membered monocyclic heterocyclyl containing at least one heteroatom selected from N, O, and S, wherein the 4- to 10-membered heterocyclyl is substituted with one or more R^{Vb}.

In some embodiments, at least one R^{A} is 4- to 10-membered monocyclic heterocyclyl containing one N (e.g., azetidinyl, pyrrolidinyl, or piperidinyl).

In some embodiments, at least one R^{A} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Vb}.

In some embodiments, at least one R^{A} is azetidinyl.

In some embodiments, at least one R^{A} is azetidinyl substituted with one or more R^{Vb}.

In some embodiments, at least one R^{A} is

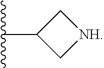 [SMILES: *C1CNC1]

In some embodiments, at least one R^{A} is

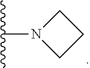 [SMILES: *N1CCC1]

In some embodiments, at least one R^{A} is

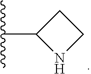 [SMILES: *C1CCN1]

In some embodiments, at least one R^{A} is pyrrolidinyl.

In some embodiments, at least one R^{A} is

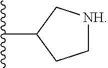 [SMILES: *C1CCNC1]

In some embodiments, at least one R^{A} is

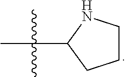 [SMILES: *C1CCCN1]

In some embodiments, at least one R^{A} is

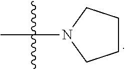 [SMILES: *N1CCCC1]

In some embodiments, at least one R^{A} is piperidinyl.

In some embodiments, at least one R^{A} is

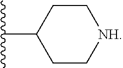 [SMILES: *C1CCNCC1]

In some embodiments, at least one R^{A} is

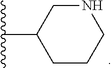 [SMILES: *C1CCCNC1]

In some embodiments, at least one R^{A} is

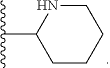 [SMILES: *C1CCCCN1]

In some embodiments, at least one R^{A} is

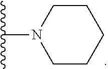 [SMILES: *N1CCCCC1]

In some embodiments, at least one R^{A} is 4- to 10-membered fused bicyclic heterocyclyl containing one N.

In some embodiments, at least one R^{A} is 4- to 10-membered fused bicyclic heterocyclyl containing one N, wherein the 4- to 10-membered fused bicyclic heterocyclyl is substituted with one or more R^{Vb}.

In some embodiments, at least one R^{A} is

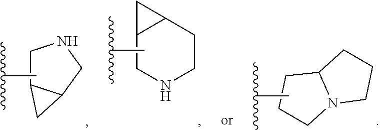

In some embodiments, at least one R^{A} is

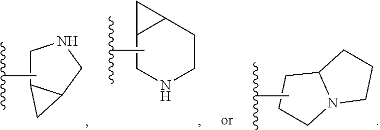

substituted with one or more R^{Vb}.

In some embodiments, at least one R^{A} is

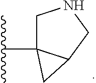 [SMILES: *C12CNCC1C2]

In some embodiments, at least one R^{A} is

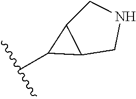 [SMILES: *C1C2CNCC12]

In some embodiments, at least one R^{A} is H

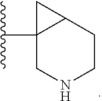 [SMILES: *C12CNCCC1C2]

In some embodiments, at least one R^{A} is

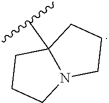 [SMILES: *C12CCCN1CCC2]

In some embodiments, at least one R^{A} is 5- to 10-membered bridged bicyclic heterocyclyl containing one N.

In some embodiments, at least one R^{A} is 5- to 10-membered bridged bicyclic heterocyclyl containing one N, wherein the 5- to 10-membered bridged bicyclic heterocyclyl is substituted with one or more R^{Vb}.

In some embodiments, at least one R^{A} is

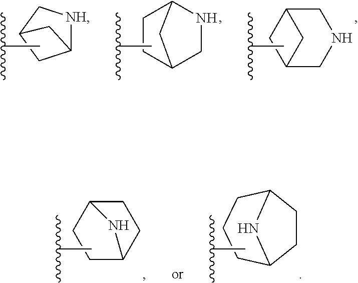

In some embodiments, at least one R^{A} is

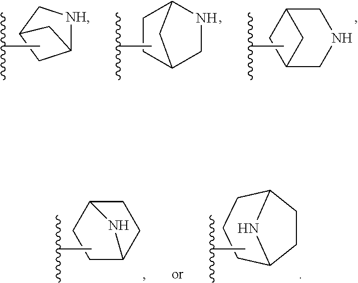

substituted with one or more R^{Vb}.

In some embodiments, at least one R^{A} is

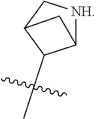 [SMILES: *C1C2CNC1C2]

In some embodiments, at least one R^{A} is

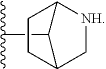 [SMILES: *C1C2CCC1NC2]

In some embodiments, at least one R^{A} is

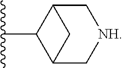 [SMILES: *C1C2CNCC1C2]

In some embodiments, at least one R^{A} is

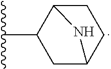 [SMILES: *C1CC2CCC1N2]

In some embodiments, at least one R^{A} is

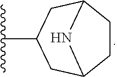 [SMILES: *C1CC2CCC(C1)N2]

In some embodiments, at least one R^{A} is

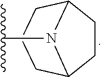 [SMILES: *N1C2CCCC1CC2]

In some embodiments, at least one R^{A} is 5- to 10-membered spiro bicyclic heterocyclyl containing one N.

In some embodiments, at least one R^{A} is 5- to 10-membered spiro bicyclic heterocyclyl containing one N, wherein the 5- to 10-membered spiro bicyclic heterocyclyl is substituted with one or more R^{Vb}.

In some embodiments, at least one R^{A} is

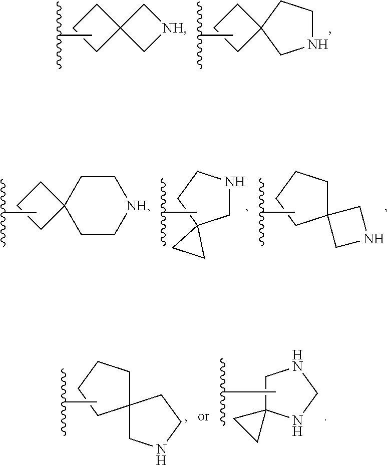

In some embodiments, at least one R^{A} is

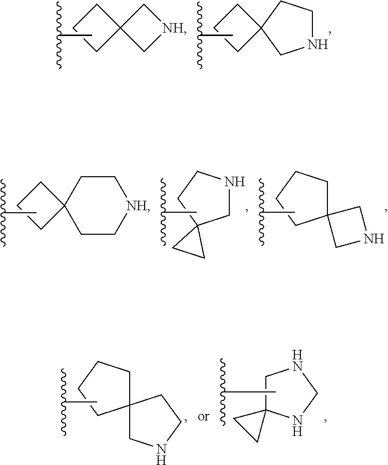

substituted with one or more R^{Vb}.

In some embodiments, at least one R^{A} is

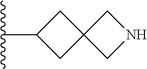 [SMILES: *C1CC2(CNC2)C1]

In some embodiments, at least one R^{A} is

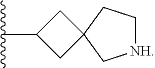 [SMILES: *C1CC2(CCNC2)C1]

In some embodiments, at least one R^{A} is

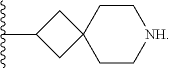 [SMILES: *C1CC2(CCNCC2)C1]

In some embodiments, at least one R^{A} is

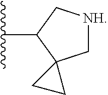 [SMILES: *C1CNCC12CC2]

In some embodiments, at least one R^{A} is

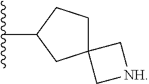 [SMILES: *C1CCC2(CNC2)C1]

In some embodiments, at least one R^{A} is

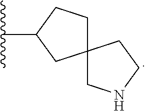 [SMILES: *C1CCC2(CCNC2)C1]

In some embodiments, at least one R^{A} is 4- to 10-membered monocyclic heterocyclyl containing one O (e.g., oxetanyl, tetrahydrofuranyl, or tetrahydropyranyl).

In some embodiments, at least one R^{A} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Vb}.

In some embodiments, at least one R^{A} is oxetanyl.

In some embodiments, at least one R^{A} is

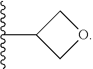 [SMILES: *C1COC1]

In some embodiments, at least one R^{A} is

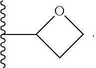 [SMILES: *C1CCO1]

In some embodiments, at least one R^{A} is tetrahydrofuranyl.

In some embodiments, at least one R^{A} is

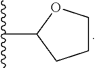 [SMILES: *C1CCCO1]

In some embodiments, at least one R^{A} is

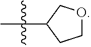 [SMILES: *C1CCOC1]

In some embodiments, at least one R^{A} is tetrahydropyranyl.

In some embodiments, at least one R^{A} is

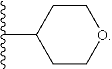 [SMILES: *C1CCOCC1]

In some embodiments, at least one R^{A} is

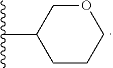 [SMILES: *C1CCCOC1]

In some embodiments, at least one R^{A} is

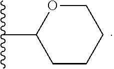 [SMILES: *C1CCCCO1]

In some embodiments, at least one R^{A} is 5- to 10-membered bridged bicyclic heterocyclyl containing one O.

In some embodiments, at least one R^{A} is 5- to 10-membered bridged bicyclic heterocyclyl containing one O, wherein the 5- to 10-membered bridged bicyclic heterocyclyl is substituted with one or more R^{Vb}.

In some embodiments, at least one R^{A} is 4- to 10-membered fused bicyclic heterocyclyl containing one O.

In some embodiments, at least one R^{A} is 4- to 10-membered fused bicyclic heterocyclyl containing one O, wherein the 4- to 10-membered fused heterocyclyl is substituted with one or more R^{Vb}.

In some embodiments, at least one R^{A} is 5- to 10-membered spiro bicyclic heterocyclyl containing one O.

In some embodiments, at least one R^{A} is 5- to 10-membered spiro bicyclic heterocyclyl containing one O, wherein the 5- to 10-membered spiro bicyclic heterocyclyl is substituted with one or more R^{Vb}.

In some embodiments, at least one R^{A} is

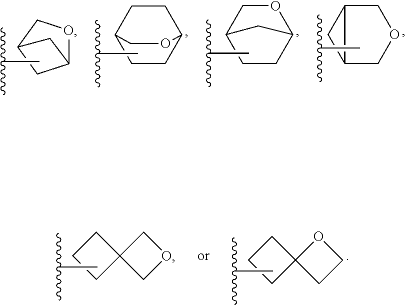

In some embodiments, at least one R^{A} is

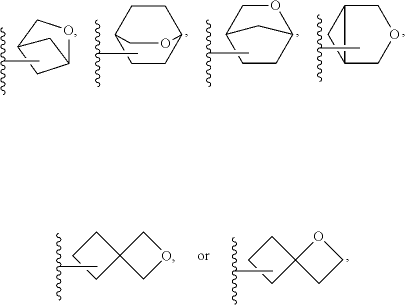

substituted with one or more R^{Vb}.

In some embodiments, at least one R^{A} is

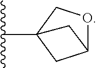 [SMILES: *C12COC(C1)C2]

In some embodiments, at least one R^{A} is

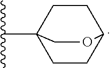 [SMILES: *C12CCC(CC1)OC2]

In some embodiments, at least one R^{A} is

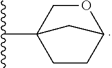 [SMILES: *C12CCC(C1)OC2]

In some embodiments, at least one R^{A} is

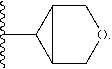 [SMILES: *C1C2COCC12]

In some embodiments, at least one R^{A} is

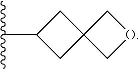 [SMILES: *C1CC2(COC2)C1]

In some embodiments, at least one R^{A} is

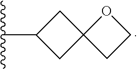 [SMILES: *C1CC2(CCO2)C1]

In some embodiments, at least one R^{A} is 4- to 10-membered monocyclic heterocyclyl containing one S (e.g., tetrahydrothiophenyl).

In some embodiments, at least one R^{A} is 4- to 10-membered monocyclic heterocyclyl containing one S (e.g., tetrahydrothiophenyl), wherein the 4- to 10-membered monocyclic heterocyclyl is substituted with one or more R^{Vb}.

In some embodiments, at least one R^{A} is tetrahydrothiophenyl.

In some embodiments, at least one R^{A} is

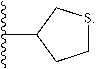 [SMILES: *C1CCSC1]

In some embodiments, at least one R^{A} is

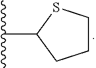 [SMILES: *C1CCCS1]

In some embodiments, at least one R^{A} is 4- to 10-membered heterocyclyl (e.g., monocyclic, fused bicyclic, bridged bicyclic, or spiro bicyclic) containing two heteroatoms selected from N, O, and S.

In some embodiments, at least one R^{A} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Vb}.

In some embodiments, at least one R^{A} is 4- to 10-membered monocyclic heterocyclyl containing two heteroatoms selected from N, O, and S.

In some embodiments, at least one R^{A} is 4- to 10-membered monocyclic heterocyclyl containing two heteroatoms selected from N, O, and S, wherein the 4- to 10-membered monocyclic heterocyclyl is substituted with one or more R^{Vb}.

In some embodiments, at least one R^{A} is 4- to 10-membered fused bicyclic heterocyclyl containing two heteroatoms selected from N, O, and S.

In some embodiments, at least one R^{A} is 4- to 10-membered fused bicyclic heterocyclyl containing two heteroatoms selected from N, O, and S, wherein the 4- to 10-membered fused bicyclic heterocyclyl is substituted with one or more R^{Vb}.

In some embodiments, at least one R^{A} is 5- to 10-membered bridged bicyclic heterocyclyl containing two heteroatoms selected from N, O, and S.

In some embodiments, at least one R^{A} is 5- to 10-membered bridged bicyclic heterocyclyl containing two heteroatoms selected from N, O, and S, wherein the 5- to 10-membered bridged bicyclic heterocyclyl is substituted with one or more R^{Vb}.

In some embodiments, at least one R^{A} is 5- to 10-membered spiro bicyclic heterocyclyl containing two heteroatoms selected from N, O, and S.

In some embodiments, at least one R^{A} is 5- to 10-membered spiro bicyclic heterocyclyl containing two heteroatoms selected from N, O, and S, wherein the 5- to 10-membered spiro bicyclic heterocyclyl is substituted with one or more R^{Vb}.

In some embodiments, at least one R^{A} is

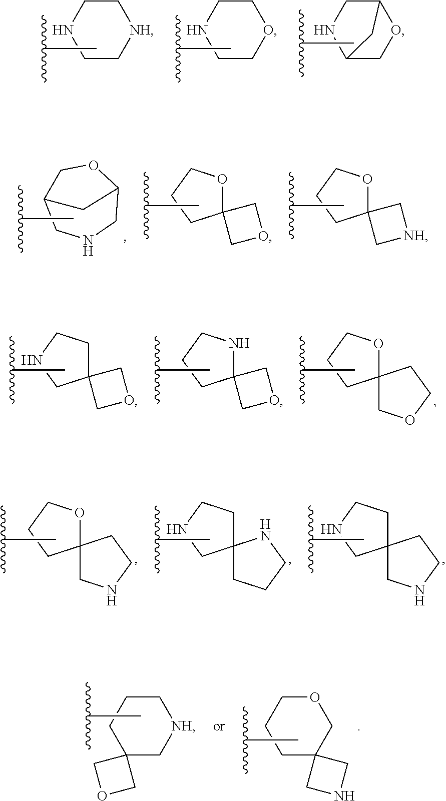

In some embodiments, at least one R^{A} is

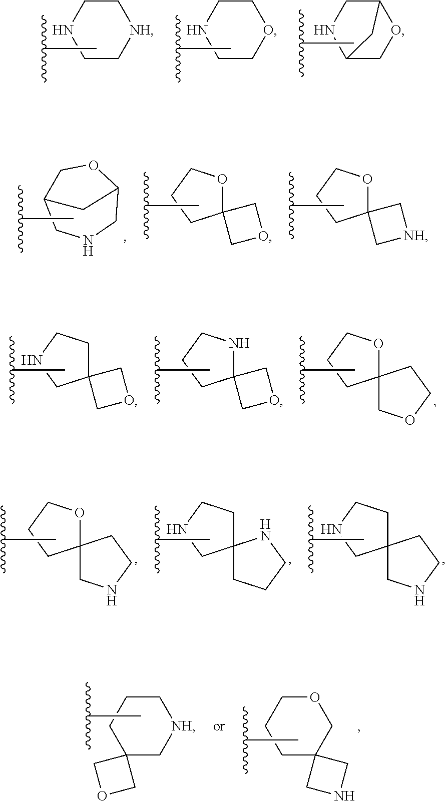

substituted with one or more R^{Vb}.

In some embodiments, at least one R^{A} is

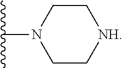 [SMILES: *N1CCNCC1]

In some embodiments, at least one R^{A} is

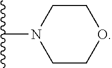 [SMILES: *N1CCOCC1]

In some embodiments, at least one R^{A} is

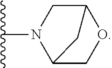 [SMILES: *N1CC2CC1CO2]

In some embodiments, at least one R^{A} is

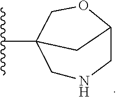 [SMILES: *C12CNCC(C1)OC2]

In some embodiments, at least one R^{A} is

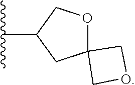 [SMILES: *C1COC2(COC2)C1]

In some embodiments, at least one R^{A} is

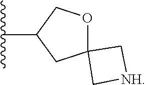 [SMILES: *C1COC2(CNC2)C1]

In some embodiments, at least one R^{A} is

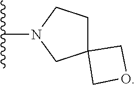 [SMILES: *N1CCC2(COC2)C1]

In some embodiments, at least one R^{A} is

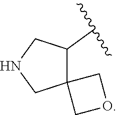 [SMILES: *C1CNCC12COC2]

In some embodiments, at least one R^{A} is

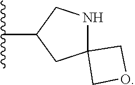 [SMILES: *C1CNC2(COC2)C1]

In some embodiments, at least one R^{A} is

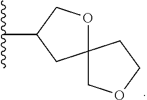 [SMILES: *C1COC2(CCOC2)C1]

In some embodiments, at least one R^{A} is H

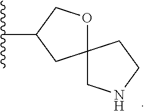 [SMILES: *C1COC2(CCNC2)C1]

In some embodiments, at least one R^{A} is

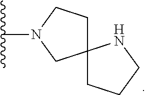 [SMILES: *N1CCC2(CCCN2)C1]

In some embodiments, at least one R^{A} is H

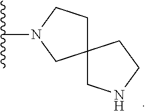 [SMILES: *N1CCC2(CCNC2)C1]

In some embodiments, at least one R^{A} is

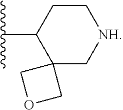 [SMILES: *C1CCNCC12COC2]

In some embodiments, at least one R^{A} is

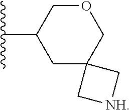 [SMILES: *C1COCC2(CNC2)C1]

In some embodiments, at least one R^{A} is C₆-C₁₀ aryl (e.g., phenyl).

In some embodiments, at least one R^{A} is C₆-C₁₀ aryl (e.g., phenyl) substituted with one or more R^{Vb}.

In some embodiments, at least one R^{A} is phenyl.

In some embodiments, at least one R^{A} is 5- to 10-membered heteroaryl.

In some embodiments, at least one R^{A} is 5- to 10-membered heteroaryl substituted with one or more R^{Vb}.

In some embodiments, at least one R^{A} is 5- to 10-membered heteroaryl containing at least one heteroatom selected from N, O, and S.

In some embodiments, at least one R^{A} is 5- to 10-membered heteroaryl containing at least one heteroatom selected from N, O, and S, wherein the heteroaryl is substituted with one or more R^{Vb}.

In some embodiments, at least one R^{A} is 5-membered heteroaryl containing at least one heteroatom selected from N, O, and S.

In some embodiments, at least one R^{A} is 5-membered heteroaryl containing at least one heteroatom selected from N, O, and S, wherein the heteroaryl is substituted with one or more R^{Vb}.

In some embodiments, at least one R^{A} is 5-membered heteroaryl containing one heteroatom selected from N, O, and S (e.g., pyrrolyl, furanyl, or thiophenyl).

In some embodiments, at least one R^{A} is 5-membered heteroaryl containing one heteroatom selected from N, O, and S (e.g., pyrrolyl, furanyl, or thiophenyl), wherein the heteroaryl is substituted with one or more R^{Vb}.

In some embodiments, at least one R^{A} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{A} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Vb}.

In some embodiments, at least one R^{A} is oxazolyl.

In some embodiments, at least one R^{A} is

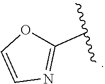 [SMILES: *c1ncco1]

In some embodiments, at least one R^{A} is thiazolyl.

In some embodiments, at least one R^{A} is

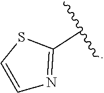 [SMILES: *c1nccs1]

In some embodiments, at least one R^{A} is pyrazolyl.

In some embodiments, at least one R^{A} is

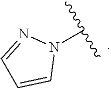 [SMILES: *n1cccn1]

In some embodiments, at least one R^{A} is

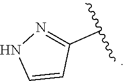 [SMILES: *c1cc[nH]n1]

In some embodiments, at least one R^{A} is isoxazolyl.

In some embodiments, at least one R^{A} is isothiazolyl.

In some embodiments, at least one R^{A} is imidazolyl.

In some embodiments, at least one R^{A} is 5-membered heteroaryl containing three heteroatoms selected from N, O, and S (e.g., triazolyl, oxadiazolyl, thiadiazolyl, or oxathiadiazolyl).

In some embodiments, at least one R^{A} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{Vb}.

In some embodiments, at least one R^{A} is triazolyl.

In some embodiments, at least one R^{A} is

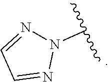 [SMILES: *n1nccn1]

In some embodiments, at least one R^{A} is

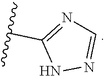 [SMILES: *c1ncn[nH]1]

In some embodiments, at least one R^{A} is

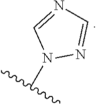 [SMILES: *n1cncn1]

In some embodiments, at least one R^{A} is oxadiazolyl.

In some embodiments, at least one R^{A} is

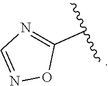 [SMILES: *c1ncno1]

In some embodiments, at least one R^{A} is thiadiazolyl.

In some embodiments, at least one R^{A} is

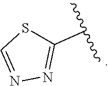 [SMILES: *c1nncs1]

In some embodiments, at least one R^{A} is

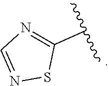 [SMILES: *c1ncns1]

In some embodiments, at least one R^{A} is 5-membered heteroaryl containing four heteroatoms selected from N, O, and S (e.g., tetrazolyl or oxatriazolyl).

In some embodiments, at least one R^{A} is 5-membered heteroaryl containing four heteroatoms selected from N, O, and S (e.g., tetrazolyl or oxatriazolyl), wherein the heteroaryl is substituted with one or more R^{Vb}.

In some embodiments, at least one R^{A} is tetrazolyl.

In some embodiments, at least one R^{A} is

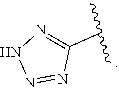 [SMILES: *c1nn[nH]n1]

In some embodiments, at least one R^{A} is oxatriazolyl.

In some embodiments, at least one R^{A} is 6-membered heteroaryl containing at least one heteroatom selected from N, O, and S.

In some embodiments, at least one R^{A} is 6-membered heteroaryl containing at least one heteroatom selected from N, O, and S, wherein the heteroaryl is substituted with one or more R^{Vb}.

In some embodiments, at least one R^{A} is 6-membered heteroaryl containing one heteroatom selected from N, O, and S (e.g., pyridinyl, pyranyl, or thiopyranyl).

In some embodiments, at least one R^{A} is 6-membered heteroaryl containing one heteroatom selected from N, O, and S (e.g., pyridinyl, pyranyl, or thiopyranyl), wherein the heteroaryl is substituted with one or more R^{Vb}.

In some embodiments, at least one R^{A} is pyridinyl.

In some embodiments, at least one R^{A} is

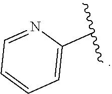 [SMILES: *c1ccccn1]

In some embodiments, at least one R^{A} is 6-membered heteroaryl containing two heteroatoms selected from N, O, and S (e.g., pyrimidinyl, pyridazinyl, or pyrazinyl).

In some embodiments, at least one R^{A} is 6-membered heteroaryl containing two heteroatoms selected from N, O, and S (e.g., pyrimidinyl, pyridazinyl, or pyrazinyl), wherein the heteroaryl is substituted with one or more R^{Vb}.

In some embodiments, at least one R^{A} is pyrimidinyl.

In some embodiments, at least one R^{A} is

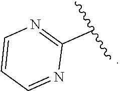 [SMILES: *c1ncccn1]

In some embodiments, at least one R^{A} is

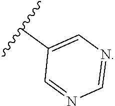 [SMILES: *c1cncnc1]

In some embodiments, at least one R^{A} is pyridazinyl.

In some embodiments, at least one R^{A} is

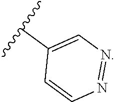 [SMILES: *c1ccnnc1]

In some embodiments, at least one R^{A} is pyrazinyl.

In some embodiments, at least one R^{A} is 9-membered heteroaryl containing at least one heteroatom selected from N, O, and S.

In some embodiments, at least one R^{A} is 9-membered heteroaryl containing at least one heteroatom selected from N, O, and S, wherein the heteroaryl is substituted with one or more R^{Vb}.

In some embodiments, at least one R^{A} is

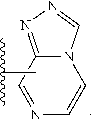

In some embodiments, at least one R^{A} is

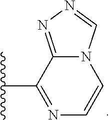 [SMILES: *c1nccn2cnnc12]

### Variable R^{B}

In some embodiments, at least one R^{B} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Vc}.

In some embodiments, at least one R^{B} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl.

In some embodiments, at least one R^{B} is C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is substituted with one or more R^{V}.

In some embodiments, at least one R^{B} is C₁-C₆ alkyl (e.g., methyl, ethyl, n-propyl, isopropyl, n-butyl, t-butyl, isobutyl, or sec-butyl).

In some embodiments, at least one R^{B} is C₁-C₆ alkyl (e.g., methyl, ethyl, n-propyl, isopropyl, n-butyl, t-butyl, isobutyl, or sec-butyl) substituted with one or more R^{Vc}.

In some embodiments, at least one R^{B} is methyl.

In some embodiments, at least one R^{B} is ethyl.

In some embodiments, at least one R^{B} is n-propyl.

In some embodiments, at least one R^{B} is isopropyl.

In some embodiments, at least one R^{B} is n-butyl.

In some embodiments, at least one R^{B} is C₂-C₆ alkenyl.

In some embodiments, at least one R^{B} is C₂-C₆ alkenyl substituted with one or more R^{Vc}.

In some embodiments, at least one R^{B} is C₂-C₆ alkynyl.

In some embodiments, at least one R^{B} is C₂-C₆ alkynyl substituted with one or more R^{Vc}.

In some embodiments, at least one R^{B} is C₃-C₆ cycloalkyl.

In some embodiments, at least one R^{B} is C₃-C₆ cycloalkyl substituted with one or more R^{Vc}.

In some embodiments, at least one R^{B} is C₃-C₆ monocyclic cycloalkyl.

In some embodiments, at least one R^{B} is C₃-C₆ monocyclic cycloalkyl substituted with one or more R^{Vc}.

In some embodiments, at least one R^{B} is cyclopropyl, cyclobutyl, cyclopentyl, cyclohexyl, cycloheptyl, or cyclooctyl.

In some embodiments, at least one R^{B} is C₃-C₆ bicyclic cycloalkyl.

In some embodiments, at least one R^{B} is C₃-C₆ bicyclic cycloalkyl substituted with one or more R^{Vc}.

In some embodiments, at least one R^{B} is bicyclocyclobutyl, bicyclocyclopentyl, or bicyclocyclohexyl.

In some embodiments, at least one R^{B} is C₄-C₆ fused bicyclic cycloalkyl.

In some embodiments, at least one R^{B} is C₄-C₆ fused bicyclic cycloalkyl substituted with one or more R^{Vc}.

In some embodiments, at least one R^{B} is

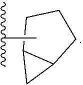

In some embodiments, at least one R^{B} is

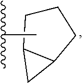

substituted with one or more R^{Vc}.

In some embodiments, at least one R^{B} is

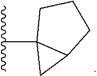 [SMILES: *C12CCCC1C2]

In some embodiments, at least one R^{B} is

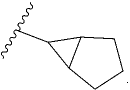 [SMILES: *C1C2CCCC12]

In some embodiments, at least one R^{B} is C₅-C₆ bridged bicyclic cycloalkyl.

In some embodiments, at least one R^{B} is C₅-C₆ bridged bicyclic cycloalkyl substituted with one or more R^{Vc}.

In some embodiments, at least one R^{B} is

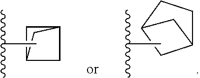

In some embodiments, at least one R^{B} is

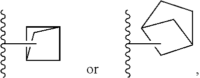

substituted with one or more R^{Vc}.

In some embodiments, at least one R^{B} is

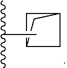

In some embodiments, at least one R^{B} is

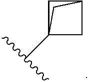 [SMILES: *C12CC(C1)C2]

In some embodiments, at least one R^{B} is

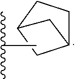

In some embodiments, at least one R^{B} is

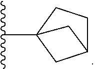 [SMILES: *C12CCC(C1)C2]

In some embodiments, at least one R^{B} is C₅-C₆ spiro bicyclic cycloalkyl.

In some embodiments, at least one R^{B} is C₅-C₆ spiro bicyclic cycloalkyl substituted with one or more R^{Vc}.

In some embodiments, at least one R^{B} is spiro[2.2]pentyl or spiro[2.3]hexyl.

In some embodiments, at least one R^{B} is spiro[2.2]pentyl or spiro[2.3]hexyl, substituted with one or more R^{Vc}.

In some embodiments, at least one R^{B} is

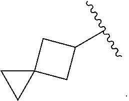 [SMILES: *C1CC2(CC2)C1]

In some embodiments, at least one R^{B} is

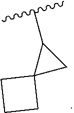 [SMILES: *C1CC12CCC2]

In some embodiments, at least one R^{B} is 4- to 10-membered heterocyclyl.

In some embodiments, at least one R^{B} is 4- to 10-membered heterocyclyl substituted with one or more R^{Vc}.

In some embodiments, at least one R^{B} is 4- to 10-membered monocyclic heterocyclyl.

In some embodiments, at least one R^{B} is 4- to 10-membered monocyclic heterocyclyl substituted with one or more R^{Vc}.

In some embodiments, at least one R^{B} is 4- to 10-membered bicyclic heterocyclyl.

In some embodiments, at least one R^{B} is 4- to 10-membered bicyclic heterocyclyl substituted with one or more R^{Vc}.

In some embodiments, at least one R^{B} is 4- to 10-membered fused bicyclic heterocyclyl.

In some embodiments, at least one R^{B} is 4- to 10-membered fused bicyclic heterocyclyl substituted with one or more R^{Vc}.

In some embodiments, at least one R^{B} is 5- to 10-membered bridged bicyclic heterocyclyl.

In some embodiments, at least one R^{B} is 5- to 10-membered bridged bicyclic heterocyclyl substituted with one or more R^{Vc}.

In some embodiments, at least one R^{B} is 5- to 10-membered spiro bicyclic heterocyclyl.

In some embodiments, at least one R^{B} is 5- to 10-membered spiro bicyclic heterocyclyl substituted with one or more R^{Vc}.

In some embodiments, at least one R^{B} is 4- to 10-membered heterocyclyl containing at least one heteroatom selected from N, O, and S.

In some embodiments, at least one R^{B} is 4- to 10-membered heterocyclyl containing at least one heteroatom selected from N, O, and S, wherein the 4- to 10-membered heterocyclyl is substituted with one or more R^{Vc}.

In some embodiments, at least one R^{B} is 4- to 10-membered monocyclic heterocyclyl containing at least one heteroatom selected from N, O, and S.

In some embodiments, at least one R^{B} is 4- to 10-membered monocyclic heterocyclyl containing at least one heteroatom selected from N, O, and S, wherein the 4- to 10-membered heterocyclyl is substituted with one or more R^{Vc}.

In some embodiments, at least one R^{B} is 4- to 10-membered monocyclic heterocyclyl containing one N (e.g., azetidinyl, pyrrolidinyl, or piperidinyl).

In some embodiments, at least one R^{B} is 4- to 10-membered monocyclic heterocyclyl containing one N (e.g., azetidinyl, pyrrolidinyl, or piperidinyl), wherein the 4- to 10-membered monocyclic heterocyclyl is substituted with one or more R^{Vc}.

In some embodiments, at least one R^{B} is azetidinyl.

In some embodiments, at least one R^{B} is azetidinyl substituted with one or more R^{Vc}.

In some embodiments, at least one R^{B} is

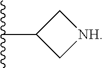 [SMILES: *C1CNC1]

In some embodiments, at least one R^{B} is

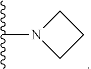 [SMILES: *N1CCC1]

In some embodiments, at least one R^{B} is

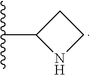 [SMILES: *C1CCN1]

In some embodiments, at least one R^{B} is pyrrolidinyl.

In some embodiments, at least one R^{B} is

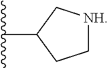 [SMILES: *C1CCNC1]

In some embodiments, at least one R^{B} is

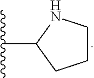 [SMILES: *C1CCCN1]

In some embodiments, at least one R^{B} is

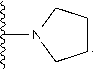 [SMILES: *N1CCCC1]

In some embodiments, at least one R^{B} is piperidinyl.

In some embodiments, at least one R^{B} is

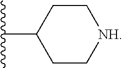 [SMILES: *C1CCNCC1]

In some embodiments, at least one R^{B} is

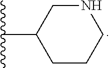 [SMILES: *C1CCCNC1]

In some embodiments, at least one R^{B} is

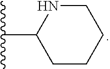 [SMILES: *C1CCCCN1]

In some embodiments, at least one R^{B} is

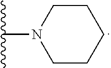 [SMILES: *N1CCCCC1]

In some embodiments, at least one R^{B} is 4- to 10-membered fused bicyclic heterocyclyl containing one N.

In some embodiments, at least one R^{B} is 4- to 10-membered fused bicyclic heterocyclyl containing one N, wherein the 4- to 10-membered fused bicyclic heterocyclyl is substituted with one or more R^{Vc}.

In some embodiments, at least one R^{B} is

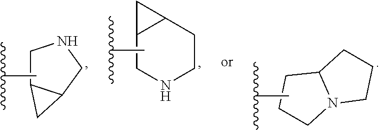

In some embodiments, at least one R^{B} is

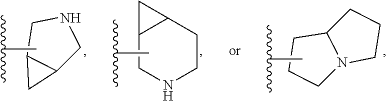

substituted with one or more R^{Vc}.

In some embodiments, at least one R^{B} is

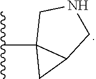 [SMILES: *C12CNCC1C2]

In some embodiments, at least one R^{B} is

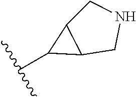 [SMILES: *C1C2CNCC12]

In some embodiments, at least one R^{B} is

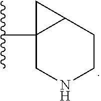 [SMILES: *C12CNCCC1C2]

In some embodiments, at least one R^{B} is

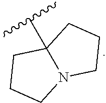 [SMILES: *C12CCCN1CCC2]

In some embodiments, at least one R^{B} is 5- to 10-membered bridged bicyclic heterocyclyl containing one N.

In some embodiments, at least one R^{B} is 5- to 10-membered bridged bicyclic heterocyclyl containing one N, wherein the 5- to 10-membered bridged bicyclic heterocyclyl is substituted with one or more R^{Vc}.

In some embodiments, at least one R^{B} is

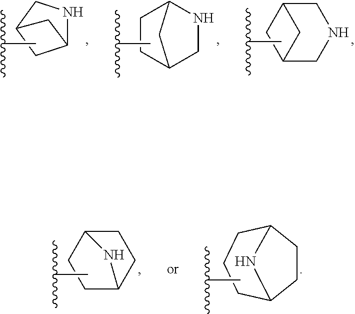

In some embodiments, at least one R^{B} is

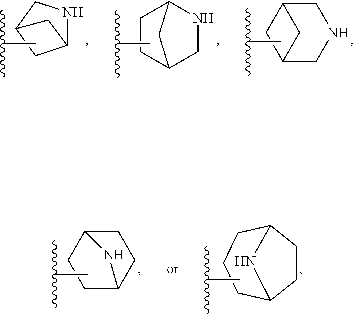

substituted with one or more R^{Vc}.

In some embodiments, at least one R^{B} is

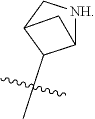 [SMILES: *C1C2CNC1C2]

In some embodiments, at least one R^{B} is

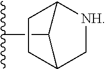 [SMILES: *C1C2CCC1NC2]

In some embodiments, at least one R^{B} is

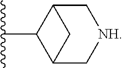 [SMILES: *C1C2CNCC1C2]

In some embodiments, at least one R^{B} is

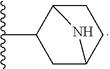 [SMILES: *C1CC2CCC1N2]

In some embodiments, at least one R^{B} is

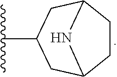 [SMILES: *C1CC2CCC(C1)N2]

In some embodiments, at least one R^{B} is

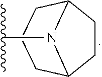 [SMILES: *N1C2CCCC1CC2]

In some embodiments, at least one R^{B} is 5- to 10-membered spiro bicyclic heterocyclyl containing one N.

In some embodiments, at least one R^{B} is 5- to 10-membered spiro bicyclic heterocyclyl containing one N, wherein the 5- to 10-membered spiro bicyclic heterocyclyl is substituted with one or more R^{Vc}.

In some embodiments, at least one R^{B} is

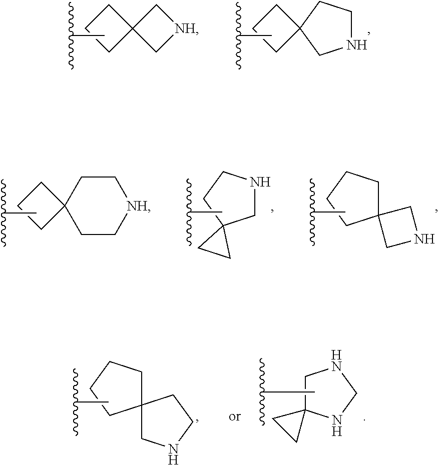

In some embodiments, at least one R^{B} is

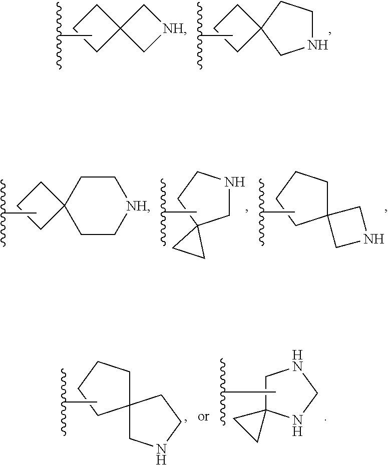

substituted with one or more R^{Vc}.

In some embodiments, at least one R^{B} is

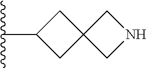 [SMILES: *C1CC2(CNC2)C1]

In some embodiments, at least one R^{B} is

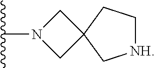 [SMILES: *N1CC2(CCNC2)C1]

In some embodiments, at least one R^{B} is

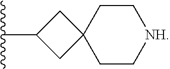 [SMILES: *C1CC2(CCNCC2)C1]

In some embodiments, at least one R^{B} is

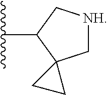 [SMILES: *C1CNCC12CC2]

In some embodiments, at least one R^{B} is

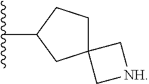 [SMILES: *C1CCC2(CNC2)C1]

In some embodiments, at least one R^{B} is

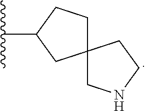 [SMILES: *C1CCC2(CCNC2)C1]

In some embodiments, at least one R^{B} is 4- to 10-membered monocyclic heterocyclyl containing one O (e.g., oxetanyl, tetrahydrofuranyl, or tetrahydropyranyl).

In some embodiments, at least one R^{B} is 4- to 10-membered monocyclic heterocyclyl containing one O (e.g., oxetanyl, tetrahydrofuranyl, or tetrahydropyranyl), wherein the 4- to 10-membered monocyclic heterocyclyl is substituted with one or more R^{Vc}.

In some embodiments, at least one R^{B} is oxetanyl.

In some embodiments, at least one R^{B} is

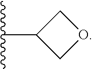 [SMILES: *C1COC1]

In some embodiments, at least one R^{B} is

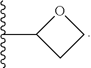 [SMILES: *C1CCO1]

In some embodiments, at least one R^{B} is tetrahydrofuranyl.

In some embodiments, at least one R^{B} is

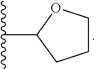 [SMILES: *C1CCCO1]

In some embodiments, at least one R^{B} is

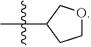 [SMILES: *C1CCOC1]

In some embodiments, at least one R^{B} is tetrahydropyranyl.

In some embodiments, at least one R^{B} is

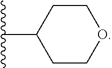 [SMILES: *C1CCOCC1]

In some embodiments, at least one R^{B} is

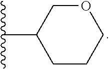 [SMILES: *C1CCCOC1]

In some embodiments, at least one R^{B} is

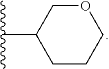 [SMILES: *C1CCCOC1]

In some embodiments, at least one R^{B} is 5- to 10-membered bridged bicyclic heterocyclyl containing one O.

In some embodiments, at least one R^{B} is 5- to 10-membered bridged bicyclic heterocyclyl containing one O, wherein the 5- to 10-membered bridged bicyclic heterocyclyl is substituted with one or more R^{Vc}.

In some embodiments, at least one R^{B} is 4- to 10-membered fused bicyclic heterocyclyl containing one O.

In some embodiments, at least one R^{B} is 4- to 10-membered fused bicyclic heterocyclyl containing one O, wherein the 4- to 10-membered fused heterocyclyl is substituted with one or more R^{Vc}.

In some embodiments, at least one R^{B} is 5- to 10-membered spiro bicyclic heterocyclyl containing one O.

In some embodiments, at least one R^{B} is 5- to 10-membered spiro bicyclic heterocyclyl containing one O, wherein the is 5- to 10-membered spiro bicyclic heterocyclyl is substituted with one or more R^{Vc}.

In some embodiments, at least one R^{B} is

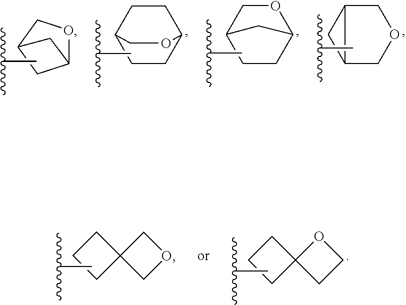

In some embodiments, at least one R^{B} is

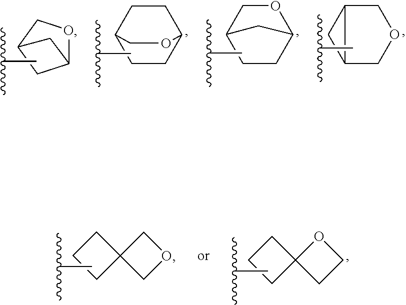

substituted with one or more R^{Vc}.

In some embodiments, at least one R^{B} is

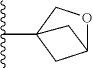 [SMILES: *C12COC(C1)C2]

In some embodiments, at least one R^{B} is

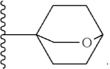 [SMILES: *C12CCC(CC1)OC2]

In some embodiments, at least one R^{B} is

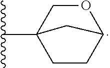 [SMILES: *C12CCC(C1)OC2]

In some embodiments, at least one R^{B} is

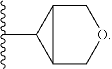 [SMILES: *C1C2COCC12]

In some embodiments, at least one R^{B} is

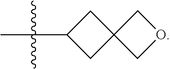 [SMILES: *C1CC2(COC2)C1]

In some embodiments, at least one R^{B} is

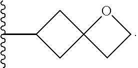 [SMILES: *C1CC2(CCO2)C1]

In some embodiments, at least one R^{B} is 4- to 10-membered monocyclic heterocyclyl containing one S (e.g., tetrahydrothiophenyl).

In some embodiments, at least one R^{B} is 4- to 10-membered monocyclic heterocyclyl containing one S (e.g., tetrahydrothiophenyl), wherein the 4- to 10-membered monocyclic heterocyclyl is substituted with one or more R^{Vc}.

In some embodiments, at least one R^{B} is tetrahydrothiophenyl.

In some embodiments, at least one R^{B} is

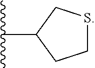 [SMILES: *C1CCSC1]

In some embodiments, at least one R^{B} is

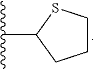 [SMILES: *C1CCCS1]

In some embodiments, at least one R^{B} is 4- to 10-membered heterocyclyl (e.g., monocyclic, fused bicyclic, bridged bicyclic, or spiro bicyclic) containing two heteroatoms selected from N, O, and S.

In some embodiments, at least one R^{B} is 4- to 10-membered heterocyclyl (e.g., monocyclic, fused bicyclic, bridged bicyclic, or spiro bicyclic) containing two heteroatoms selected from N, O, and S, wherein the 4- to 10-membered heterocyclyl is substituted with one or more R^{Vc}.

In some embodiments, at least one R^{B} is 4- to 10-membered monocyclic heterocyclyl containing two heteroatoms selected from N, O, and S.

In some embodiments, at least one R^{B} is 4- to 10-membered monocyclic heterocyclyl containing two heteroatoms selected from N, O, and S, wherein the 4- to 10-membered monocyclic heterocyclyl is substituted with one or more R^{Vc}.

In some embodiments, at least one R^{B} is 4- to 10-membered fused bicyclic heterocyclyl containing two heteroatoms selected from N, O, and S.

In some embodiments, at least one R^{B} is 4- to 10-membered fused bicyclic heterocyclyl containing two heteroatoms selected from N, O, and S, wherein the 4- to 10-membered fused bicyclic heterocyclyl is substituted with one or more R^{Vc}.

In some embodiments, at least one R^{B} is 5- to 10-membered bridged bicyclic heterocyclyl containing two heteroatoms selected from N, O, and S.

In some embodiments, at least one R^{B} is 5- to 10-membered bridged bicyclic heterocyclyl containing two heteroatoms selected from N, O, and S, wherein the 5- to 10-membered bridged bicyclic heterocyclyl is substituted with one or more R^{Vc}.

In some embodiments, at least one R^{B} is 5- to 10-membered spiro bicyclic heterocyclyl containing two heteroatoms selected from N, O, and S.

In some embodiments, at least one R^{B} is 5- to 10-membered spiro bicyclic heterocyclyl containing two heteroatoms selected from N, O, and S, wherein the 5- to 10-membered spiro bicyclic heterocyclyl is substituted with one or more R^{Vc}.

In some embodiments, at least one R^{B} is

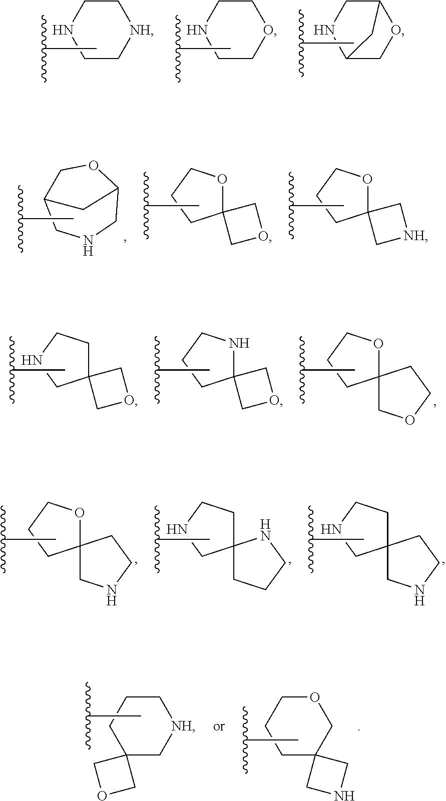

In some embodiments, at least one R^{B} is

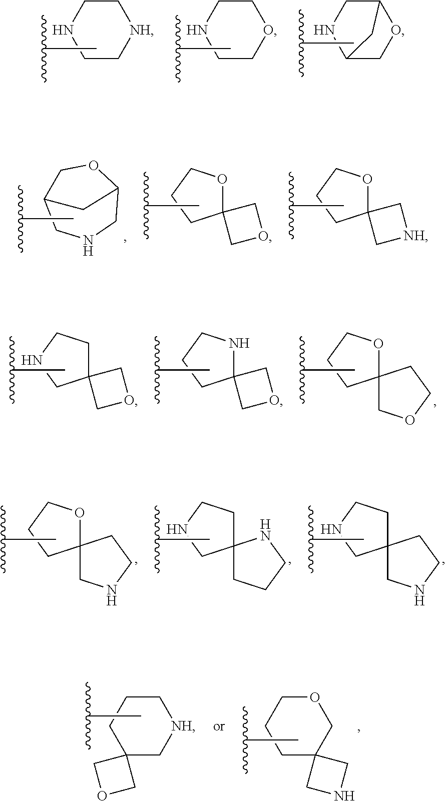

substituted with one or more R^{Vc}.

In some embodiments, at least one R^{B} is

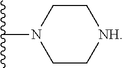 [SMILES: *N1CCNCC1]

In some embodiments, at least one R^{B} is

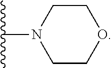 [SMILES: *N1CCOCC1]

In some embodiments, at least one R^{B} is

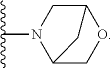 [SMILES: *N1CC2CC1CO2]

In some embodiments, at least one R^{B} is H

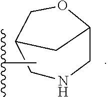

In some embodiments, at least one R^{B} is

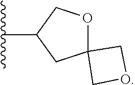 [SMILES: *C1COC2(COC2)C1]

In some embodiments, at least one R^{B} is

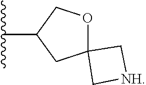 [SMILES: *C1COC2(CNC2)C1]

In some embodiments, at least one R^{B} is

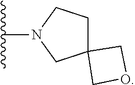 [SMILES: *N1CCC2(COC2)C1]

In some embodiments, at least one R^{B} is

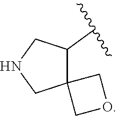 [SMILES: *C1CNCC12COC2]

In some embodiments, at least one R^{B} is

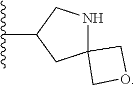 [SMILES: *C1CNC2(COC2)C1]

In some embodiments, at least one R^{B} is

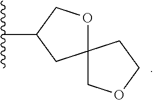 [SMILES: *C1COC2(CCOC2)C1]

In some embodiments, at least one R^{B} is

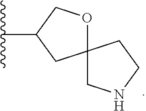 [SMILES: *C1COC2(CCNC2)C1]

In some embodiments, at least one R^{B} is

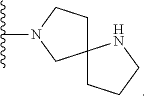 [SMILES: *N1CCC2(CCCN2)C1]

In some embodiments, at least one R^{B} is

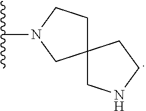 [SMILES: *N1CCC2(CCNC2)C1]

In some embodiments, at least one R^{B} is

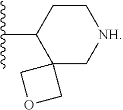 [SMILES: *C1CCNCC12COC2]

In some embodiments, at least one R^{B} is

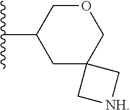 [SMILES: *C1COCC2(CNC2)C1]

In some embodiments, at least one R^{B} is C₆-C₁₀ aryl (e.g., phenyl).

In some embodiments, at least one R^{B} is C₆-C₁₀ aryl (e.g., phenyl) substituted with one or more R^{Vc}.

In some embodiments, at least one R^{B} is phenyl.

In some embodiments, at least one R^{B} is 5- to 10-membered heteroaryl.

In some embodiments, at least one R^{B} is 5- to 10-membered heteroaryl substituted with one or more R^{Vc}.

In some embodiments, at least one R^{B} is 5- to 10-membered heteroaryl containing at least one heteroatom selected from N, O, and S.

In some embodiments, at least one R^{B} is 5- to 10-membered heteroaryl containing at least one heteroatom selected from N, O, and S, wherein the heteroaryl is substituted with one or more R^{Vc}.

In some embodiments, at least one R^{B} is 5-membered heteroaryl containing at least one heteroatom selected from N, O, and S.

In some embodiments, at least one R^{B} is 5-membered heteroaryl containing at least one heteroatom selected from N, O, and S, wherein the heteroaryl is substituted with one or more R^{Vc}.

In some embodiments, at least one R^{B} is 5-membered heteroaryl containing one heteroatom selected from N, O, and S (e.g., pyrrolyl, furanyl, or thiophenyl).

In some embodiments, at least one R^{B} is 5-membered heteroaryl containing one heteroatom selected from N, O, and S (e.g., pyrrolyl, furanyl, or thiophenyl), wherein the heteroaryl is substituted with one or more R^{Vc}.

In some embodiments, at least one R^{B} is 5-membered heteroaryl containing two heteroatoms selected from N, O, and S (e.g., oxazolyl, thiazolyl, pyrazolyl, isoxazolyl, isothiazolyl, or imidazolyl).

In some embodiments, at least one R^{B} is 5-membered heteroaryl containing two heteroatoms selected from N, O, and S (e.g., oxazolyl, thiazolyl, pyrazolyl, isoxazolyl, isothiazolyl, or imidazolyl), wherein the heteroaryl is substituted with one or more R^{Vc}.

In some embodiments, at least one R^{B} is oxazolyl.

In some embodiments, at least one R^{B} is

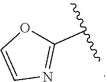 [SMILES: *c1ncco1]

In some embodiments, at least one R^{B} is thiazolyl.

In some embodiments, at least one R^{B} is

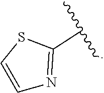 [SMILES: *c1nccs1]

In some embodiments, at least one R^{B} is pyrazolyl.

In some embodiments, at least one R^{B} is

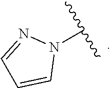 [SMILES: *n1cccn1]

In some embodiments, at least one R^{B} is

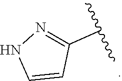 [SMILES: *c1cc[nH]n1]

In some embodiments, at least one R^{B} is isoxazolyl.

In some embodiments, at least one R^{B} is isothiazolyl.

In some embodiments, at least one R^{B} is imidazolyl.

In some embodiments, at least one R^{B} is 5-membered heteroaryl containing three heteroatoms selected from N, O, and S (e.g., triazolyl, oxadiazolyl, thiadiazolyl, or oxathiadiazolyl).

In some embodiments, at least one R^{B} is 5-membered heteroaryl containing three heteroatoms selected from N, O, and S (e.g., triazolyl, oxadiazolyl, thiadiazolyl, or oxathiadiazolyl), wherein the heteroaryl is substituted with one or more R^{V}.

In some embodiments, at least one R^{B} is triazolyl.

In some embodiments, at least one R^{B} is

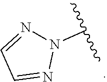 [SMILES: *n1nccn1]

In some embodiments, at least one R^{B} is

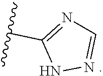 [SMILES: *c1ncn[nH]1]

In some embodiments, at least one R^{B} is

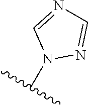 [SMILES: *n1cncn1]

In some embodiments, at least one R^{B} is oxadiazolyl.

In some embodiments, at least one R^{B} is

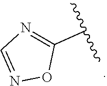 [SMILES: *c1ncno1]

In some embodiments, at least one R^{B} is thiadiazolyl.

In some embodiments, at least one R^{B} is

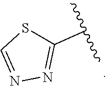 [SMILES: *c1nncs1]

In some embodiments, at least one R^{B} is

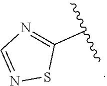 [SMILES: *c1ncns1]

In some embodiments, at least one R^{B} is 5-membered heteroaryl containing four heteroatoms selected from N, O, and S (e.g., tetrazolyl or oxatriazolyl).

In some embodiments, at least one R^{B} is 5-membered heteroaryl containing four heteroatoms selected from N, O, and S (e.g., tetrazolyl or oxatriazolyl), wherein the heteroaryl is substituted with one or more R^{Vc}.

In some embodiments, at least one R^{B} is tetrazolyl.

In some embodiments, at least one R^{B} is

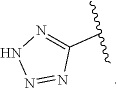 [SMILES: *c1nn[nH]n1]

In some embodiments, at least one R^{B} is oxatriazolyl.

In some embodiments, at least one R^{B} is 6-membered heteroaryl containing at least one heteroatom selected from N, O, and S.

In some embodiments, at least one R^{B} is 6-membered heteroaryl containing at least one heteroatom selected from N, O, and S, wherein the heteroaryl is substituted with one or more R^{Vc}.

In some embodiments, at least one R^{B} is 6-membered heteroaryl containing one heteroatom selected from N, O, and S (e.g., pyridinyl, pyranyl, or thiopyranyl).

In some embodiments, at least one R^{B} is 6-membered heteroaryl containing one heteroatom selected from N, O, and S (e.g., pyridinyl, pyranyl, or thiopyranyl), wherein the heteroaryl is substituted with one or more R^{Vc}.

In some embodiments, at least one R^{B} is pyridinyl.

In some embodiments, at least one R^{B} is

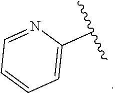 [SMILES: *c1ccccn1]

In some embodiments, at least one R^{B} is 6-membered heteroaryl containing two heteroatoms selected from N, O, and S (e.g., pyrimidinyl, pyridazinyl, or pyrazinyl).

In some embodiments, at least one R^{B} is 6-membered heteroaryl containing two heteroatoms selected from N, O, and S (e.g., pyrimidinyl, pyridazinyl, or pyrazinyl), wherein the heteroaryl is substituted with one or more R^{Vc}.

In some embodiments, at least one R^{B} is pyrimidinyl.

In some embodiments, at least one R^{B} is

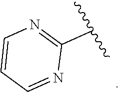 [SMILES: *c1ncccn1]

In some embodiments, at least one R^{B} is

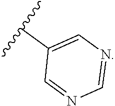 [SMILES: *c1cncnc1]

In some embodiments, at least one R^{B} is pyridazinyl.

In some embodiments, at least one R^{B} is

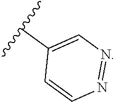 [SMILES: *c1ccnnc1]

In some embodiments, at least one R^{B} is pyrazinyl.

In some embodiments, at least one R^{B} is 9-membered heteroaryl containing at least one heteroatom selected from N, O, and S.

In some embodiments, at least one R^{B} is 9-membered heteroaryl containing at least one heteroatom selected from N, O, and S, wherein the heteroaryl is substituted with one or more R^{Vc}.

In some embodiments, at least one R^{B} is

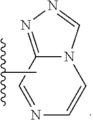

In some embodiments, at least one R^{B} is

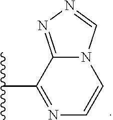 [SMILES: *c1nccn2cnnc12]

### Variable R^{C}

In some embodiments, at least one R^{C} is C₁-C₆ alkyl, C₂-C₆ alkenyl, or C₂-C₆ alkynyl.

In some embodiments, at least one R^{C} is C₁-C₆ alkyl (e.g., methyl, ethyl, n-propyl, isopropyl, n-butyl, t-butyl, isobutyl, or sec-butyl).

In some embodiments, at least one R^{C} is methyl.

In some embodiments, at least one R^{C} is ethyl.

In some embodiments, at least one R^{C} is C₂-C₆ alkenyl.

In some embodiments, at least one R^{C} C₂-C₆ alkynyl.

### Variable W

In some embodiments, W is —(C₁-C₆ alkylene)- or —(C₃-C₆ cycloalkylene)-, wherein the —(C₁-C₆ alkylene)- or —(C₃-C₆ cycloalkylene)- is optionally substituted with one or more halogen, cyano, —OH, —O(C₁-C₆ alkyl), —NH₂, —NH(C₁-C₆ alkyl), —N(C₁-C₆ alkyl)₂, or C₁-C₆ alkyl.

In some embodiments, W is —(C₁-C₆ alkylene)- or —(C₃-C₆ cycloalkylene)-.

In some embodiments, W is —(C₁-C₆ alkylene)- or —(C₃-C₆ cycloalkylene)-, wherein the —(C₁-C₆ alkylene)- or —(C₃-C₆ cycloalkylene)- is optionally substituted with one or more halogen, cyano, —OH, —O(C₁-C₆ alkyl), —NH₂, —NH(C₁-C₆ alkyl), —N(C₁-C₆ alkyl)₂, or C₁-C₆ alkyl.

In some embodiments, W is —(C₁-C₆ alkylene)- (e.g., -methylene-, -ethylene-, -n-propylene-, -isopropylene-, -n-butylene-, -t-butylene-, -isobutylene-, or -sec-butylene-).

In some embodiments, W is —(C₁-C₆ alkylene)- (e.g., -methylene-, -ethylene-, -n-propylene-, -isopropylene-, -n-butylene-, -t-butylene-, -isobutylene-, or -sec-butylene-) substituted with one or more halogen, cyano, —OH, —O(C₁-C₆ alkyl), —NH₂, —NH(C₁-C₆ alkyl), —N(C₁-C₆ alkyl)₂, or C₁-C₆ alkyl.

In some embodiments, W is —(C₃-C₆ cycloalkylene)- (e.g., cyclopropylene, cyclobutylene, cyclopentylene, or cyclohexylene).

In some embodiments, W is —(C₃-C₆ cycloalkylene)- (e.g., cyclopropylene, cyclobutylene, cyclopentylene, or cyclohexylene) substituted with one or more halogen, cyano, —OH, —O(C₁-C₆ alkyl), —NH₂, —NH(C₁-C₆ alkyl), —N(C₁-C₆ alkyl)₂, or C₁-C₆ alkyl.

In some embodiments, W is -methylene- substituted with one methyl.

In some embodiments, W is -ethylene- substituted with one methyl.

In some embodiments, W is -cyclobutylene- substituted with one methyl.

### Exemplary Embodiments of the Compounds

In some embodiments of the compound of Formula (I), (I′), (II), (II-a), (II-b), (III), (III-a), (III-b), (IV), (IV-a), (IV-b), (VIII), (VIII-a), (VIII-b), (II′), (II′-a), (II′-b), (II′-c), (II′-d), (II′-e), (II′-f), (III′), (III′-a), (III′-b), (III′-c), (III′-d), (III′-e), (III′-f), (IV′), (IV′-a), (IV′-b), (IV′-c), (IV′-d), (IV′-e), or (IV′-f), or a pharmaceutically acceptable salt thereof, R¹ is C₁-C₆ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂; and R^{2a} is C₁-C₃ alkyl optionally substituted with one or more halogen, cyano, —OH, or —NH₂. In some embodiments, R¹ is C₁-C₃ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂; and R^{2a} is C₁-C₃ alkyl optionally substituted with one or more halogen, cyano, —OH, or —NH₂. In some embodiments, R¹ is C₁-C₃ alkyl and R^{2a} is C₁-C₃ alkyl.

In some embodiments of the compound of Formula (I), (I′), (II), (II-a), (II-b), (III), (III-a), (III-b), (IV), (IV-a), (IV-b), (VIII), (VIII-a), or (VIII-b), or a pharmaceutically acceptable salt thereof, R¹ is C₁-C₆ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂; R^{2a} is C₁-C₃ alkyl optionally substituted with one or more halogen, cyano, —OH, or —NH₂; and R^{2b}, R^{2c}, and R^{2d} are each H or C₁-C₃ alkyl. In some embodiments, R¹ is C₁-C₃ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂; R^{2a} is C₁-C₃ alkyl optionally substituted with one or more halogen, cyano, —OH, or —NH₂. In some embodiments, R¹ is C₁-C₃ alkyl and R^{2a} is C₁-C₃ alkyl; and R^{2b}, R^{2c} and R^{2d} are each H or C₁-C₃ alkyl. In some embodiments, R¹ is C₁-C₃ alkyl; R^{2a} is C₁-C₃ alkyl; and R^{2b}, R^{2c}, and R^{2d} are each H.

In some embodiments of the compound of Formula (I) or (I′), or a pharmaceutically acceptable salt thereof, X is —CH₂—; m is 1; and n is 0. In some embodiments, m is 0 and n is 1. In some embodiments, m is 0 and n is 0. In some embodiments, X is —CH₂—; m is 1; n is 0; and Y is phenyl or 5- to 6-membered heteroaryl, wherein the phenyl, or 5- to 10-membered heteroaryl is optionally substituted with one or more R^{Ya}. In some embodiments, m is 0; n is 1; and Y is phenyl or 5- to 6-membered heteroaryl, wherein the phenyl, or 5- to 10-membered heteroaryl is optionally substituted with one or more R^{Ya}. In some embodiments, m is 0; n is 0; and Y is phenyl or 5- to 6-membered heteroaryl, wherein the phenyl, or 5- to 10-membered heteroaryl is optionally substituted with one or more R^{Ya}.

In some embodiments of the compound of Formula (I), (I″-i), (I′″-i), (II), (II-a), (II-b), (III), (III-a), (III-b), (IV), (IV-a), (IV-b), (V), (V-a), or (V-b), or a pharmaceutically acceptable salt thereof, T is —N(R^{T1})₂; and one R^{T1} is H or C₁-C₆ alkyl; and the other R^{T1} is C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta}. In some embodiments, T is —N(R^{T1})₂; and one R^{T1} is H or C₁-C₆ alkyl; and the other R^{T1} is C₁-C₆ alkyl, C₂-C₆ alkenyl, C₂-C₆ alkynyl, monocyclic C₃-C₈ cycloalkyl, fused bicyclic C₄-C₈ cycloalkyl, bridged bicyclic C₅-C₈ cycloalkyl, spiro bicyclic C₅-C₈ cycloalkyl, monocyclic 4- to 8-membered heterocyclyl, fused bicyclic 4- to 10-membered heterocyclyl, bridged bicyclic 5- to 10-membered heterocyclyl, spiro bicyclic 5- to 10-membered heterocyclyl, phenyl, monocyclic 5- to 6-membered heteroaryl, fused bicyclic 5- to 10-membered heteroaryl, —(C₁-C₄ alkyl)-(monocyclic C₃-C₈ cycloalkyl), —(C₁-C₄ alkyl)-(fused bicyclic C₄-C₈ cycloalkyl), —(C₁-C₄ alkyl)-(bridged bicyclic C₅-C₈ cycloalkyl), —(C₁-C₄ alkyl)-(spiro bicyclic C₅-C₈ cycloalkyl), —(C₁-C₄ alkyl)-(monocyclic 4- to 7-membered heterocyclyl), —(C₁-C₄ alkyl)-(fused bicyclic 4- to 10-membered heterocyclyl), —(C₁-C₄ alkyl)-(bridged bicyclic 5- to 10-membered heterocyclyl), —(C₁-C₄ alkyl)-(spiro bicyclic 5- to 10-membered heterocyclyl), —(C₁-C₄ alkyl)-(phenyl), —(C₁-C₄ alkyl)-(monocyclic 5- to 6-membered heteroaryl), or —(C₁-C₄ alkyl)-(fused bicyclic 5- to 10-membered heteroaryl), wherein the other R^{T1} is optionally substituted with one or more R^{Ta}. In some embodiments, T is —N(R^{T1})₂; and one R^{T1} is H or C₁-C₆ alkyl; and the other R^{T1} is C₁-C₆ alkyl, C₂-C₆ alkenyl, C₂-C₆ alkynyl, cyclopropyl, cyclobutyl, cyclopentyl, cyclohexyl, cycloheptyl, cyclooctyl,

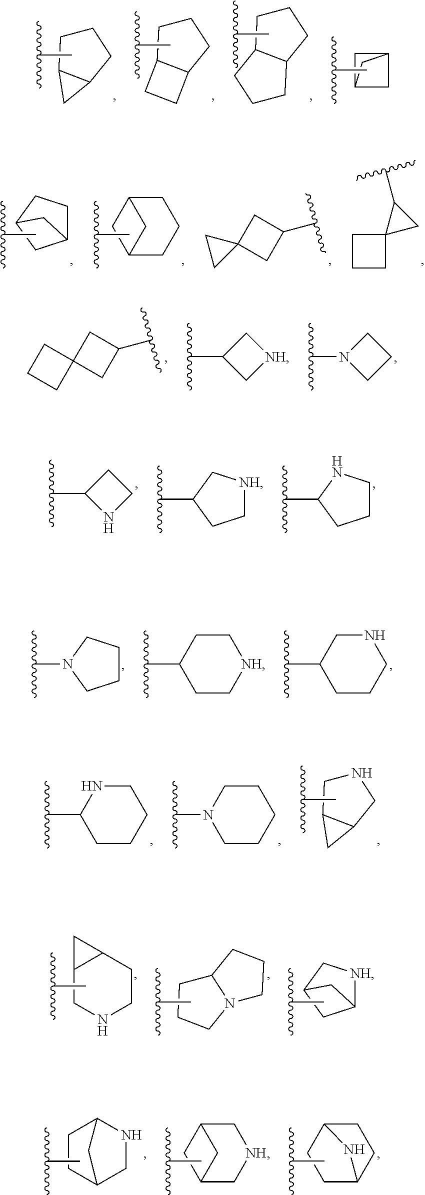

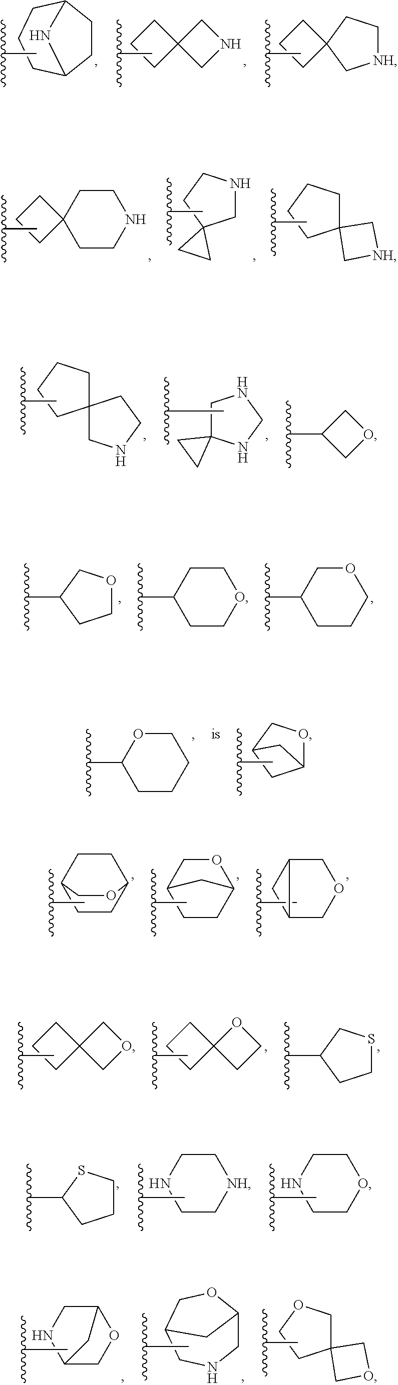

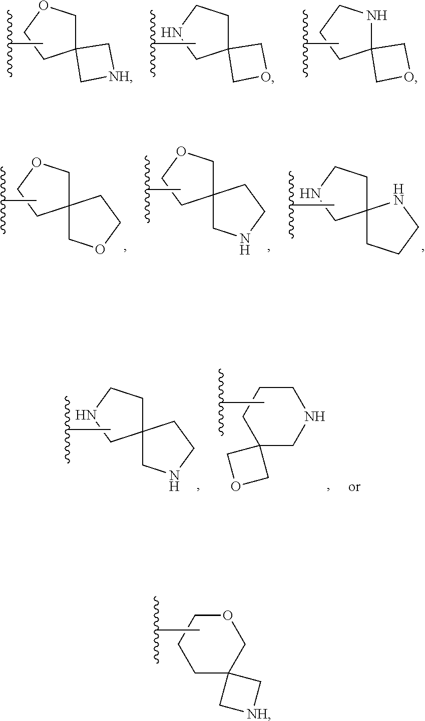

wherein the other R^{T1} is optionally substituted with one or more R^{Ta}. In some embodiments, T is —N(R^{T1})₂; and one R^{T1} is H or C₁-C₆ alkyl; and the other R^{T1} is pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, pyrazinyl, phenyl, or

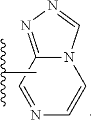

wherein the other R^{T1} is optionally substituted with one or more R^{Ta}. In some embodiments, each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{B})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb}; each R^{b} independently is C₁-C₆ alkyl optionally substituted with one or more R^{Tc}; each R^{Tb} independently is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc}; each R^{c} independently is C₁-C₆ alkyl; and each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl. In some embodiments, at least one R^{Ta} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tb}; or at least one R^{Ta} is —OR^{b}, —NHR^{b}, —N(C₁-C₆ alkyl)R^{b}, —N(R^{b})₂, —C(═O)—R^{b}, —C(═O)—OR^{b}, —C(═O)—NHR^{b}, —C(═O)—N(R^{b})₂, —S(═O)₂—R^{b}, or —S(═O)(═NH)—R^{b}, wherein R^{b} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tc}; or at least one R^{Ta} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is substituted with one group selected from C₃-C₆ cycloalkyl, 4- to 10-membered heterocyclyl, C₆-C₁₀ aryl, or 5- to 10-membered heteroaryl, and further optionally substituted with one or more R^{Tb}. In some embodiments, at least one R^{Tb} or at least one R^{Tc} is —C(═O)—OH.

In some embodiments of the compound of Formula (I), (I″-i), (I′″-i), (II), (II-a), (II-b), (III), (III-a), (III-b), (IV), (IV-a), (IV-b), (V), (V-a), or (V-b), or a pharmaceutically acceptable salt thereof, T is —NHR^{T1} or —NCH₃R^{T1}; and R^{T1} is C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Ta}. In some embodiments, T is —NHR^{T1} or —NCH₃R^{T1}; and R^{T1} is C₁-C₆ alkyl, C₂-C₆ alkenyl, C₂-C₆ alkynyl, monocyclic C₃-C₈ cycloalkyl, fused bicyclic C₄-C₈ cycloalkyl, bridged bicyclic C₅-C₈ cycloalkyl, spiro bicyclic C₅-C₈ cycloalkyl, monocyclic 4- to 8-membered heterocyclyl, fused bicyclic 4- to 10-membered heterocyclyl, bridged bicyclic 5- to 10-membered heterocyclyl, spiro bicyclic 5- to 10-membered heterocyclyl, phenyl, monocyclic 5- to 6-membered heteroaryl, fused bicyclic 5- to 10-membered heteroaryl, —(C₁-C₄ alkyl)-(monocyclic C₃-C₈ cycloalkyl), —(C₁-C₄ alkyl)-(fused bicyclic C₄-C₈ cycloalkyl), —(C₁-C₄ alkyl)-(bridged bicyclic C₅-C₈ cycloalkyl), —(C₁-C₄ alkyl)-(spiro bicyclic C₅-C₈ cycloalkyl), —(C₁-C₄ alkyl)-(monocyclic 4- to 7-membered heterocyclyl), —(C₁-C₄ alkyl)-(fused bicyclic 4- to 10-membered heterocyclyl), —(C₁-C₄ alkyl)-(bridged bicyclic 5- to 10-membered heterocyclyl), —(C₁-C₄ alkyl)-(spiro bicyclic 5- to 10-membered heterocyclyl), —(C₁-C₄ alkyl)-(phenyl), —(C₁-C₄ alkyl)-(monocyclic 5- to 6-membered heteroaryl), or —(C₁-C₄ alkyl)-(fused bicyclic 5- to 10-membered heteroaryl), wherein R^{T1} is optionally substituted with one or more R^{Ta}. In some embodiments, T is —NHR^{T1} or —NCH₃R^{T1}; and R^{T1} is C₁-C₆ alkyl, C₂-C₆ alkenyl, C₂-C₆ alkynyl, cyclopropyl, cyclobutyl, cyclopentyl, cyclohexyl, cycloheptyl, cyclooctyl,

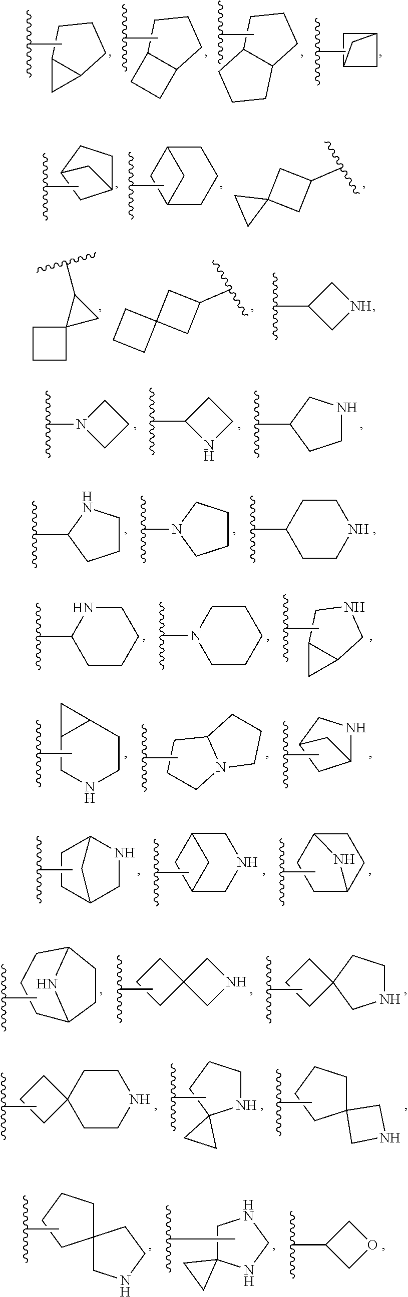

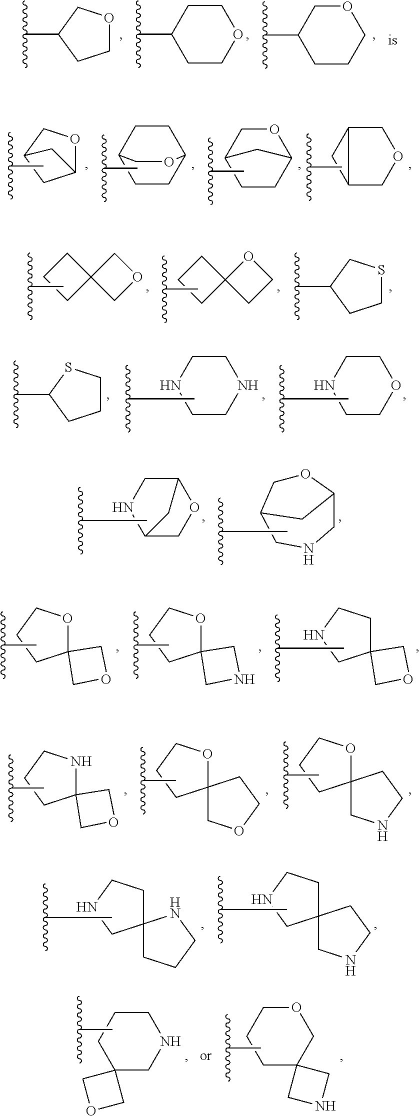

wherein R^{T1} is optionally substituted with one or more R^{Ta}. In some embodiments, T is —NHR^{T1} or —NCH₃R^{T1}; and R^{T1} is pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, pyrazinyl, phenyl, or

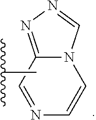

wherein R^{T1} is optionally substituted with one or more R^{Ta}. In some embodiments, each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb}; each R^{b} independently is C₁-C₆ alkyl optionally substituted with one or more R^{Tc}; each R^{Tb} independently is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc}; each R^{c} independently is C₁-C₆ alkyl; and each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl. In some embodiments, at least one R^{Ta} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tb}; or at least one R^{Ta} is —OR^{b}, —NHR^{b}, —N(C₁-C₆ alkyl)R^{b}, —N(R^{b})₂, —C(═O)—R^{b}, —C(═O)—OR^{b}, —C(═O)—NHR^{b}, —C(═O)—N(R^{b})₂, —S(═O)₂—R^{b}, or —S(═O)(═NH)—R^{b}, wherein R^{b} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tc}; or at least one R^{Ta} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is substituted with one group selected from C₃-C₆ cycloalkyl, 4- to 10-membered heterocyclyl, C₆-C₁₀ aryl, or 5- to 10-membered heteroaryl, and further optionally substituted with one or more R^{Tb}. In some embodiments, at least one R^{Tb} or at least one R^{Tc} is —C(═O)—OH.

In some embodiments of the compound of Formula (I), (I″-i), (I′″-i), (II), (II-a), (II-b), (III), (III-a), (III-b), (IV), (IV-a), (IV-b), (V), (V-a), or (V-b), or a pharmaceutically acceptable salt thereof, T is —N(R^{T2})₂; and two R^{T2}, together with the atom to which they are attached, form a monocyclic 4- to 8-membered heterocyclyl, a fused bicyclic 4- to 10-membered heterocyclyl, a bridged bicyclic 5- to 10-membered heterocyclyl, or a spiro bicyclic 5- to 10-membered heterocyclyl, each optionally substituted with one or more R^{Ta}. In some embodiments, T is —N(R^{T2})₂; and two R^{T2}, together with the atom to which they are attached, form

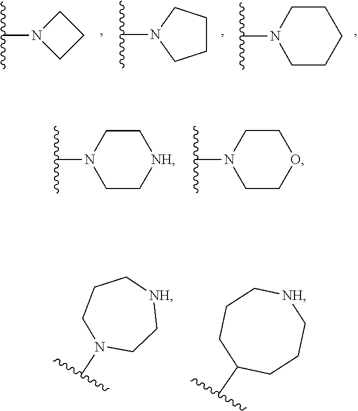

each optionally substituted with one or more R^{Ta}. In some embodiments, T is —N(R^{T2})₂; and two R^{T2}, together with the atom to which they are attached, form

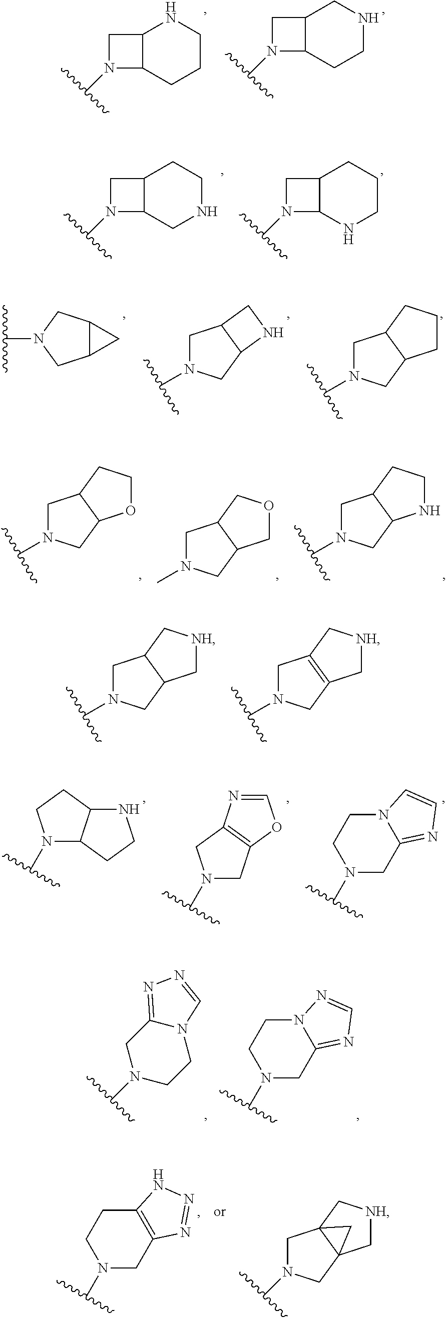

each optionally substituted with one or more R^{Ta}. In some embodiments, T is —N(R^{T2})₂; and two R^{T2}, together with the atom to which they are attached, form

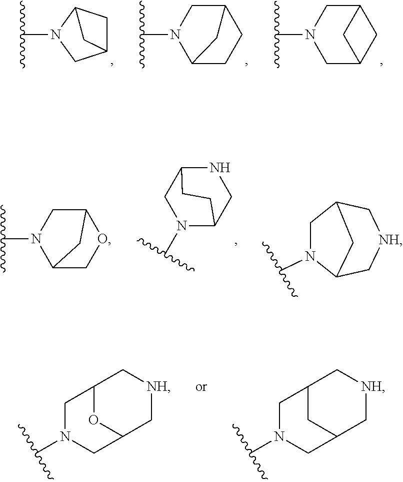

each optionally substituted with one or more R^{Ta}. In some embodiments, T is —N(R^{T2})₂; and two R^{T2}, together with the atom to which they are attached, form

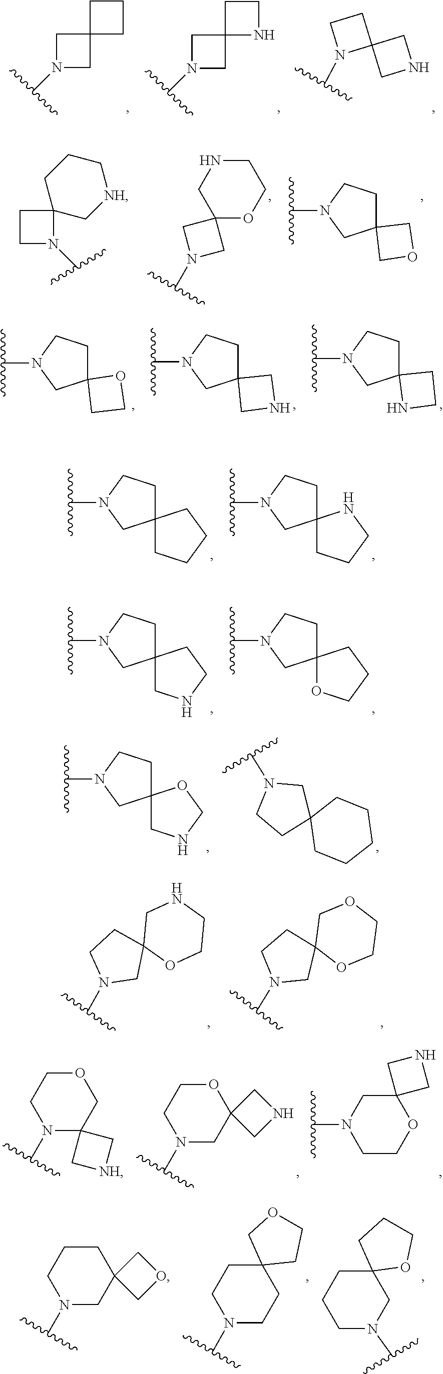

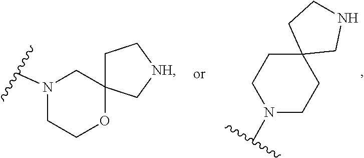 [SMILES: *N1CCOC2(CCNC2)C1 ; "or" ; *N1CCC2(CCNC2)CC1 ; ","]

each optionally substituted with one or more R^{Ta}. In some embodiments, each R^{Ta} independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tb}; each R^{b} independently is C₁-C₆ alkyl optionally substituted with one or more R^{Tc}; each R^{Tb} independently is oxo, halogen, cyano, —OH, —OR^{c}, —NH₂, —NHR^{c}, —N(R^{c})₂, —C(═O)—H, —C(═O)—R^{c}, —C(═O)—OH, —C(═O)—OR^{c}, —C(═O)—NH₂, —C(═O)—NHR^{c}, —C(═O)—N(R^{c})₂, —S(═O)₂—R^{c}, —S(═O)(═NH)—R^{c}, C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl, wherein the C₁-C₆ alkyl, C₂-C₆ alkenyl, C₂-C₆ alkynyl, C₃-C₆ cycloalkyl, 4- to 10-membered heterocyclyl, C₆-C₁₀ aryl, or 5- to 10-membered heteroaryl is optionally substituted with one or more R^{Tc}; each R^{c} independently is C₁-C₆ alkyl; and each R^{Tc} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl. In some embodiments, at least one R^{Ta} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tb}; or at least one R^{Ta} is —OR^{b}, —NHR^{b}, —N(C₁-C₆ alkyl)R^{b}, —N(R^{b})₂, —C(═O)—R^{b}, —C(═O)—OR^{b}, —C(═O)—NHR^{b}, —C(═O)—N(R^{b})₂, —S(═O)₂—R^{b}, or —S(═O)(═NH)—R^{b}, wherein R^{b} is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tc}; or at least one R^{Ta} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is substituted with one group selected from C₃-C₆ cycloalkyl, 4- to 10-membered heterocyclyl, C₆-C₁₀ aryl, or 5- to 10-membered heteroaryl, and further optionally substituted with one or more R^{Tb}. In some embodiments, at least one R^{Tb} or at least one R^{Tc} is —C(═O)—OH.

In some embodiments of the compound of Formula (I), (I″-i), (I′″-i), (II), (II-a), (II-b), (III), (III-a), (III-b), (IV), (IV-a), (IV-b), (V), (V-a), or (V-b), or a pharmaceutically acceptable salt thereof, T is —N(R^{T2})₂; two R^{T2}, together with the atom to which they are attached, form a monocyclic 4- to 8-membered heterocyclyl, a fused bicyclic 6- to 10-membered heterocyclyl, a bridged bicyclic 6- to 10-membered heterocyclyl, or a spiro bicyclic 6- to 10-membered heterocyclyl, each optionally substituted with one or more R^{Ta}; and at least one R^{Ta} is independently is oxo, halogen, cyano, —OH, —OR^{b}, —NH₂, —NHR^{b}, —N(R^{b})₂, —C(═O)—H, —C(═O)—R^{b}, —C(═O)—OH, —C(═O)—OR^{b}, —C(═O)—NH₂, —C(═O)—NHR^{b}, —C(═O)—N(R^{b})₂, —S(═O)₂—R^{b}, —S(═O)(═NH)—R^{b}, C₁-C₆ alkyl, C₂-C₆ alkenyl, C₂-C₆ alkynyl, or C₃-C₆ cycloalkyl, wherein the C₁-C₆ alkyl, C₂-C₆ alkenyl, or C₂-C₆ alkynyl is optionally substituted with one or more R^{Tb}; and at least one R^{Tb} is halogen, cyano, —OH, —O(C₁-C₆ alkyl), —NH₂, —NH(C₁-C₆ alkyl), —N(C₁-C₆ alkyl)₂, —C(═O)—H, —C(═O)—(C₁-C₆ alkyl), —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, —S(═O)₂—(C₁-C₆ alkyl), —S(═O)(═NH)—(C₁-C₆ alkyl), C₁-C₆ alkyl, C₂-C₆ alkenyl, or C₂-C₆ alkynyl. In some embodiments, at least one R^{Tb} is —C(═O)—OH.

In some embodiments of the compound of Formula (I), (I″-i), (I′″-i), (II), (II-a), (II-b), (III), (III-a), (III-b), (IV), (IV-a), (IV-b), (V), (V-a), or (V-b), or a pharmaceutically acceptable salt thereof, T is —N(R^{T2})₂; two R^{T2}, together with the atom to which they are attached, form a monocyclic 4- to 8-membered heterocyclyl, a fused bicyclic 6- to 10-membered heterocyclyl, a bridged bicyclic 6- to 10-membered heterocyclyl, or a spiro bicyclic 6- to 10-membered heterocyclyl, each optionally substituted with one or more R^{Ta}; and at least one R^{Ta} is independently is C₃-C₆ cycloalkyl, 4- to 10-membered heterocyclyl, C₆-C₁₀ aryl, or 5- to 10-membered heteroaryl, wherein the C₃-C₆ cycloalkyl, 4- to 10-membered heterocyclyl, C₆-C₁₀ aryl, or 5- to 10-membered heteroaryl is optionally substituted with one or more R^{Tb}; and at least one R^{Tb} is halogen, cyano, —OH, —O(C₁-C₆ alkyl), —NH₂, —NH(C₁-C₆ alkyl), —N(C₁-C₆ alkyl)₂, —C(═O)—H, —C(═O)—(C₁-C₆ alkyl), —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, —S(═O)₂—(C₁-C₆ alkyl), —S(═O)(═NH)—(C₁-C₆ alkyl), C₁-C₆ alkyl, C₂-C₆ alkenyl, or C₂-C₆ alkynyl. In some embodiments, at least one R^{Tb} is —C(═O)—OH.

In some embodiments of the compound of Formula (VIII), (VIII-a), or (VIII-b), or a pharmaceutically acceptable salt thereof, ring A is

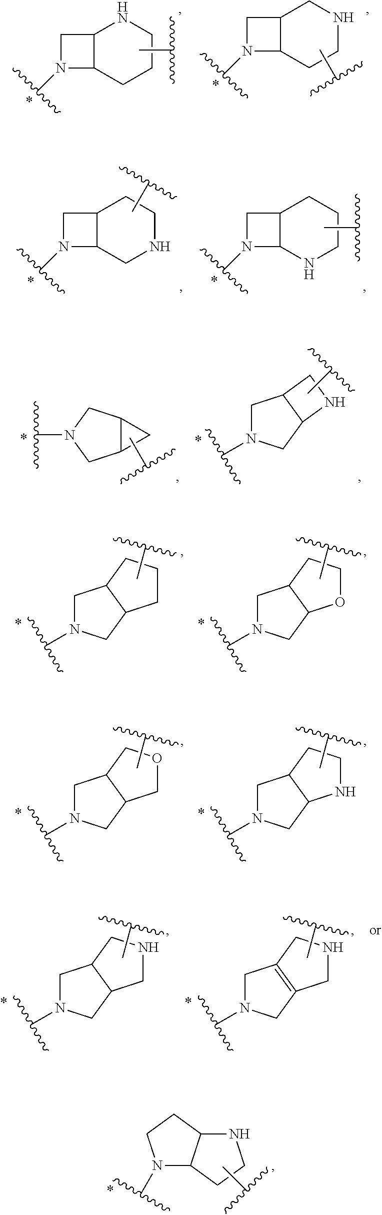

where * indicates connectivity to the carbonyl group at triazolopyridine; B is absent; and Z is absent. In some embodiments, ring A is

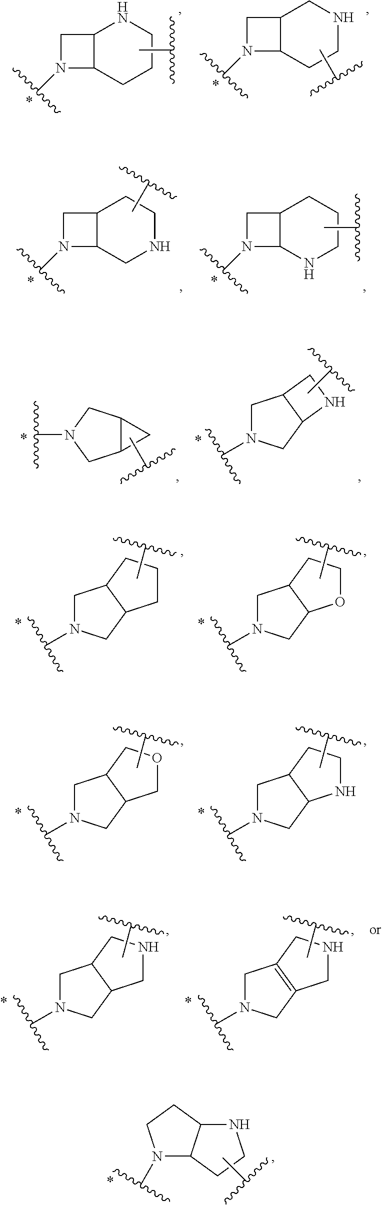

where * indicates connectivity to the carbonyl group at triazolopyridine; and ring B is C₃-C₆ cycloalkyl, 4- to 7-membered heterocyclyl, phenyl, or 5- to 6-membered heteroaryl. In some embodiments, ring A is

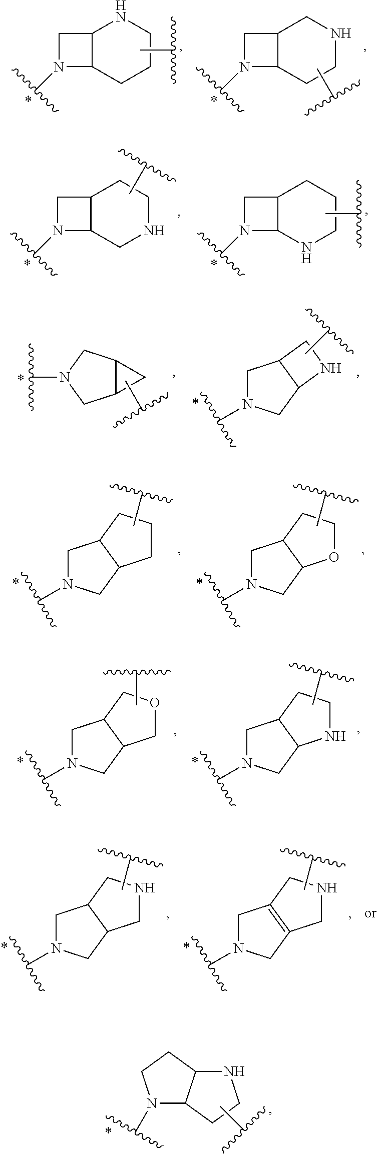

where * indicates connectivity to the carbonyl group at triazolopyridine; and ring B is pyridinyl, pyranyl, thiopyranyl, pyrimidinyl, pyridazinyl, or pyrazinyl. In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments of the compound of Formula (VIII), (VIII-a), or (VIII-b), or a pharmaceutically acceptable salt thereof, ring A is

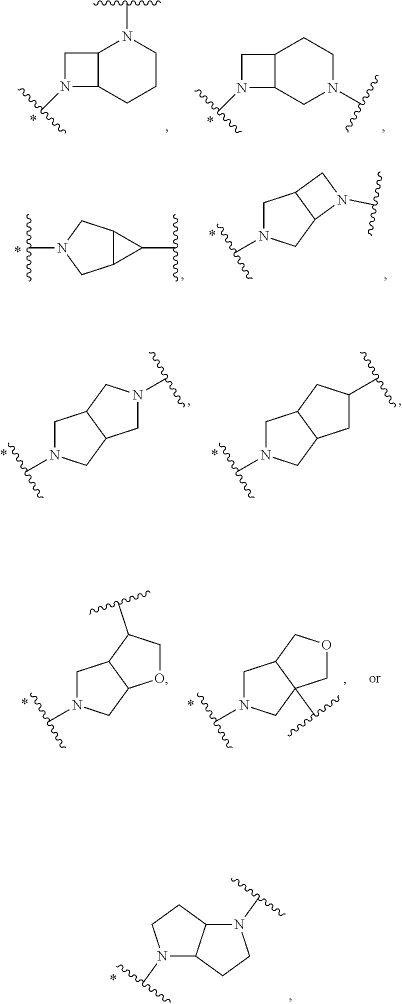

where * indicates connectivity to the carbonyl group at triazolopyridine; B is absent; and Z is absent. In some embodiments, ring A is

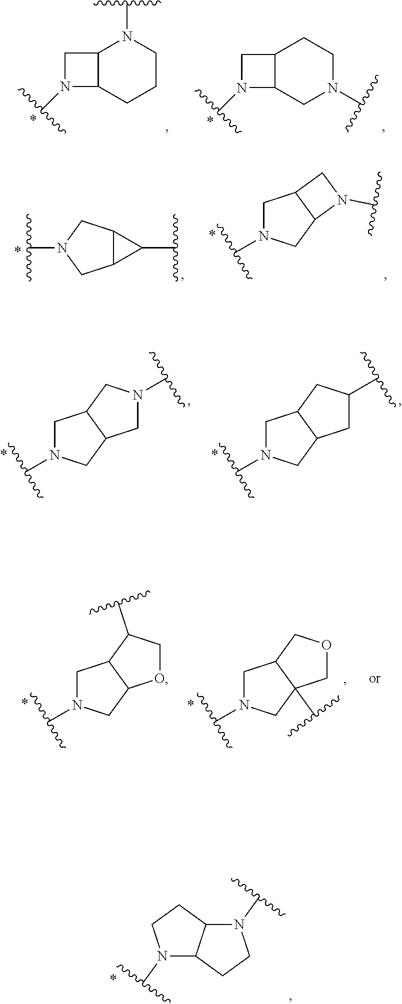

where * indicates connectivity to the carbonyl group at triazolopyridine; and ring B is C₃-C₆ cycloalkyl, 4- to 7-membered heterocyclyl, phenyl, or 5- to 6-membered heteroaryl. In some embodiments, ring A is

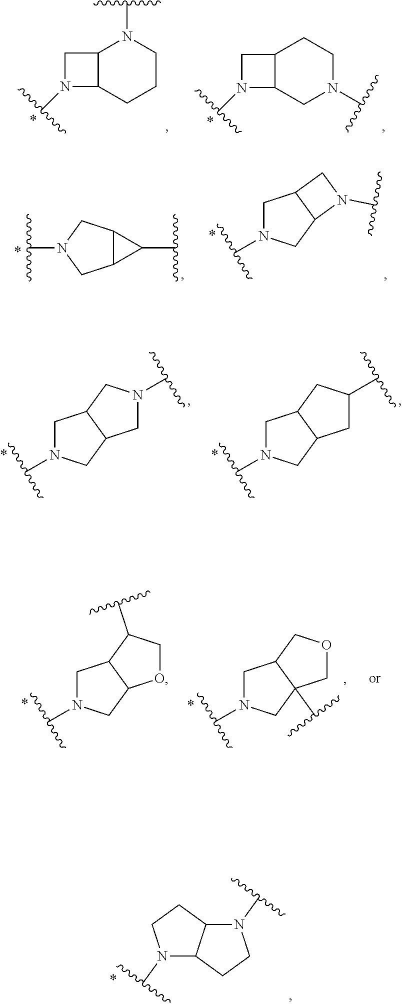

where * indicates connectivity to the carbonyl group at triazolopyridine; and ring B is pyridinyl, pyranyl, thiopyranyl, pyrimidinyl, pyridazinyl, or pyrazinyl. In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments of the compound of Formula (VIII), (VIII-a), or (VIII-b), or a pharmaceutically acceptable salt thereof, ring A is

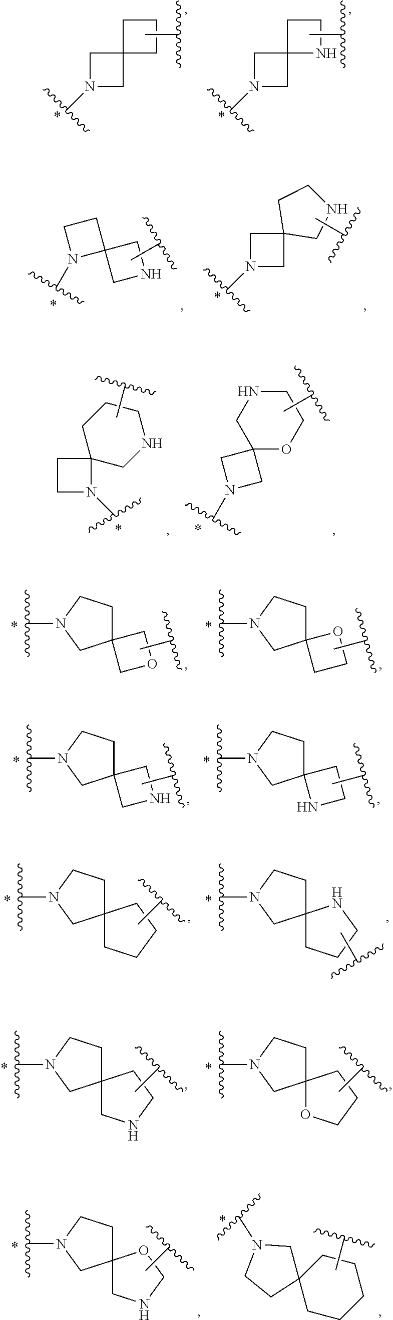

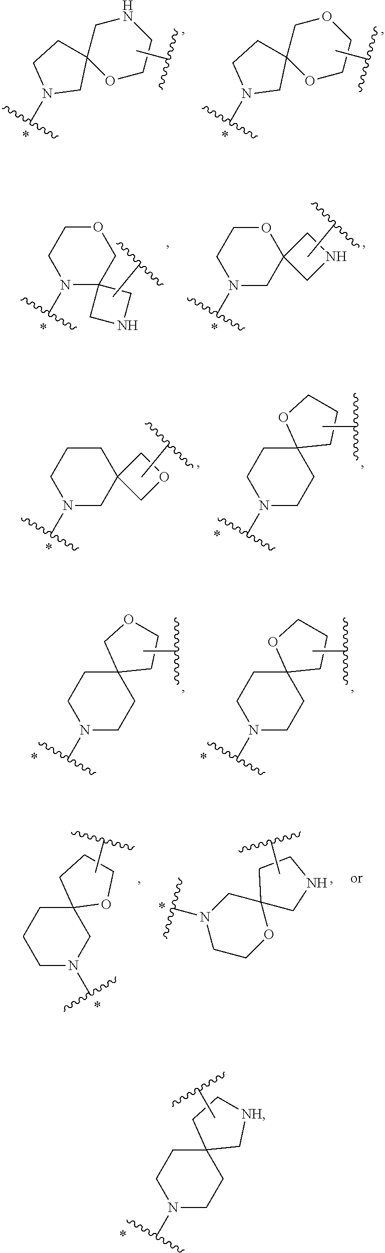

where * indicates connectivity to the carbonyl group at triazolopyridine; B is absent; and Z is absent. In some embodiments, ring A is

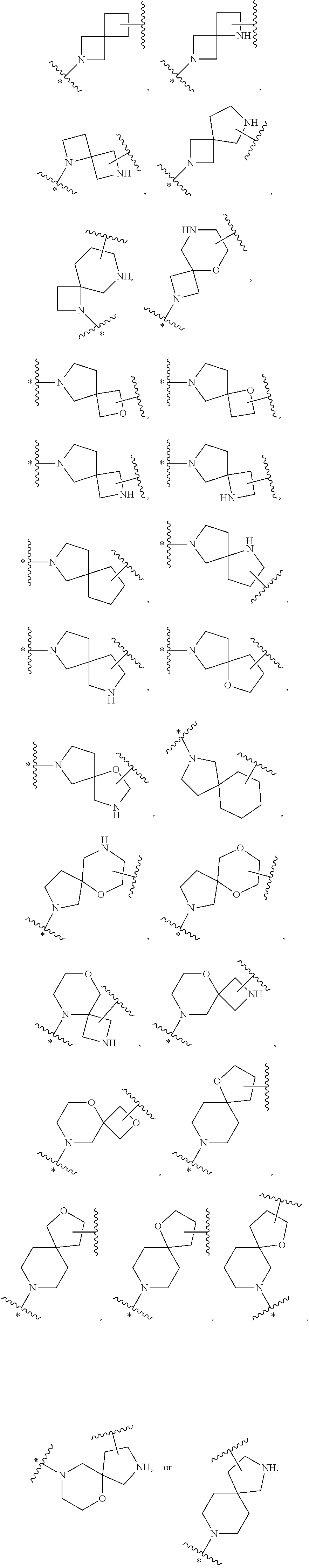

where * indicates connectivity to the carbonyl group at triazolopyridine; and ring B is C₃-C₆ cycloalkyl, 4- to 7-membered heterocyclyl, phenyl, or 5- to 6-membered heteroaryl. In some embodiments, ring A

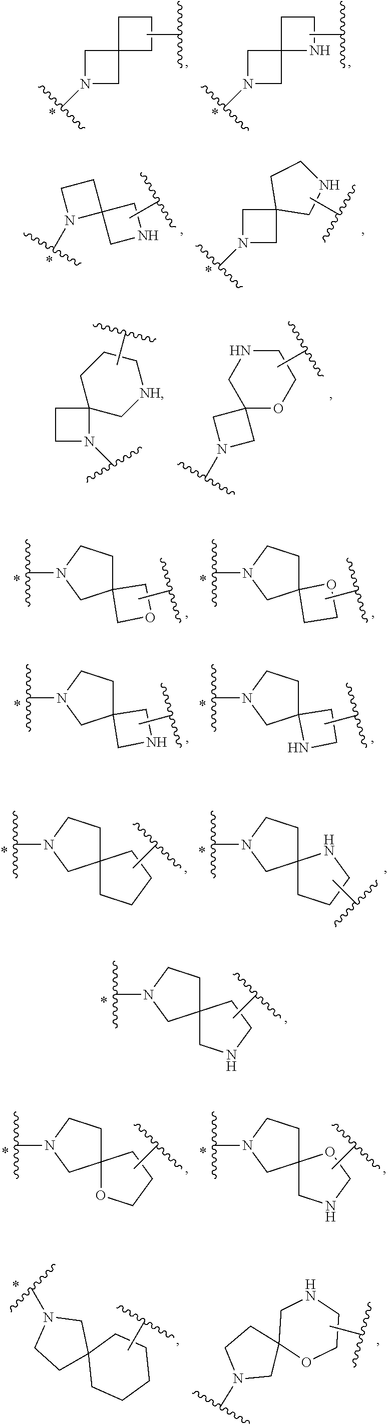

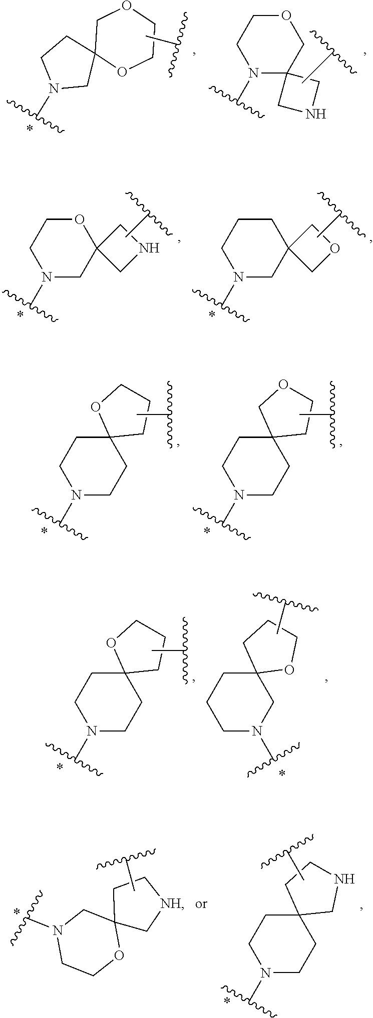

where * indicates connectivity to the carbonyl group at triazolopyridine; and ring B is pyridinyl, pyranyl, thiopyranyl, pyrimidinyl, pyridazinyl, or pyrazinyl. In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments of the compound of Formula (VIII), (VIII-a), or (VIII-b), or a pharmaceutically acceptable salt thereof, ring A is

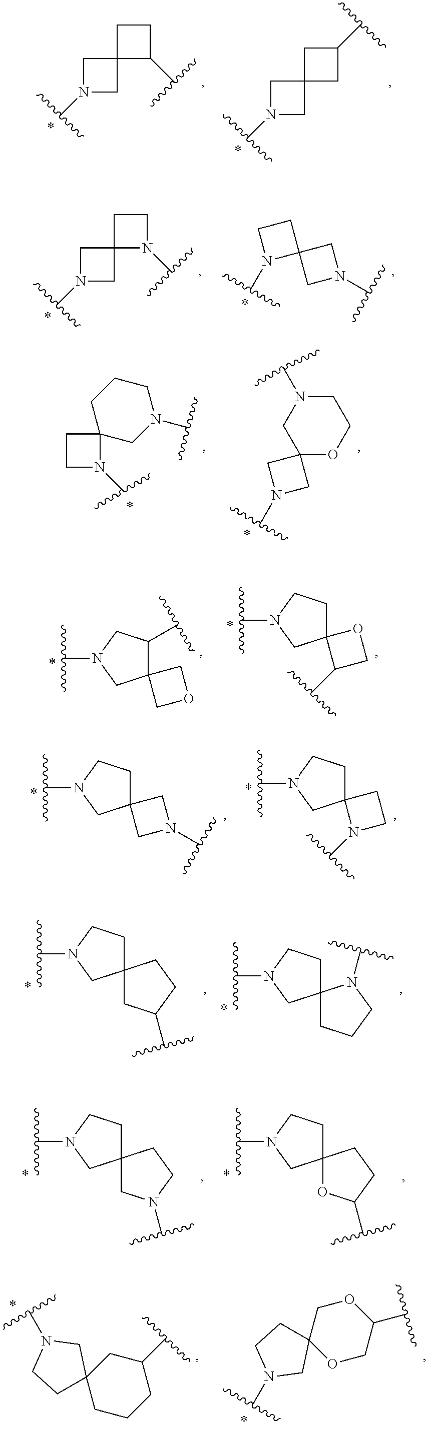

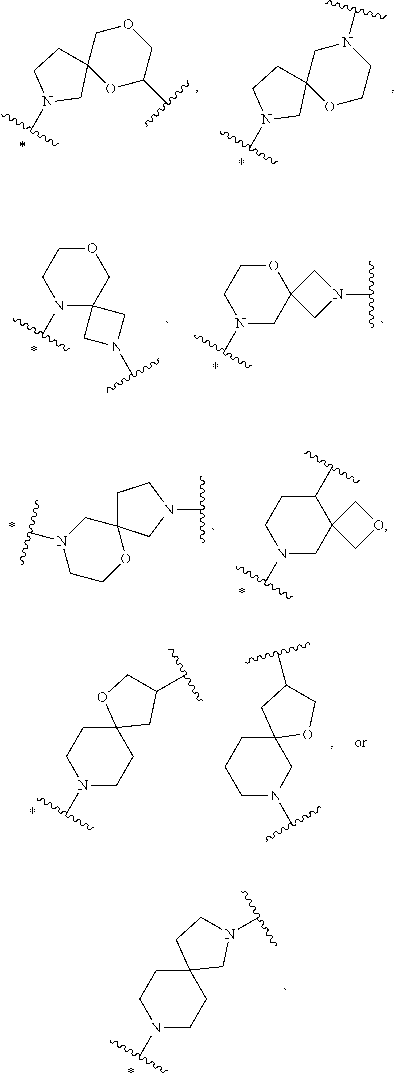

where * indicates connectivity to the carbonyl group at triazolopyridine; B is absent; and Z is absent. In some embodiments, ring A

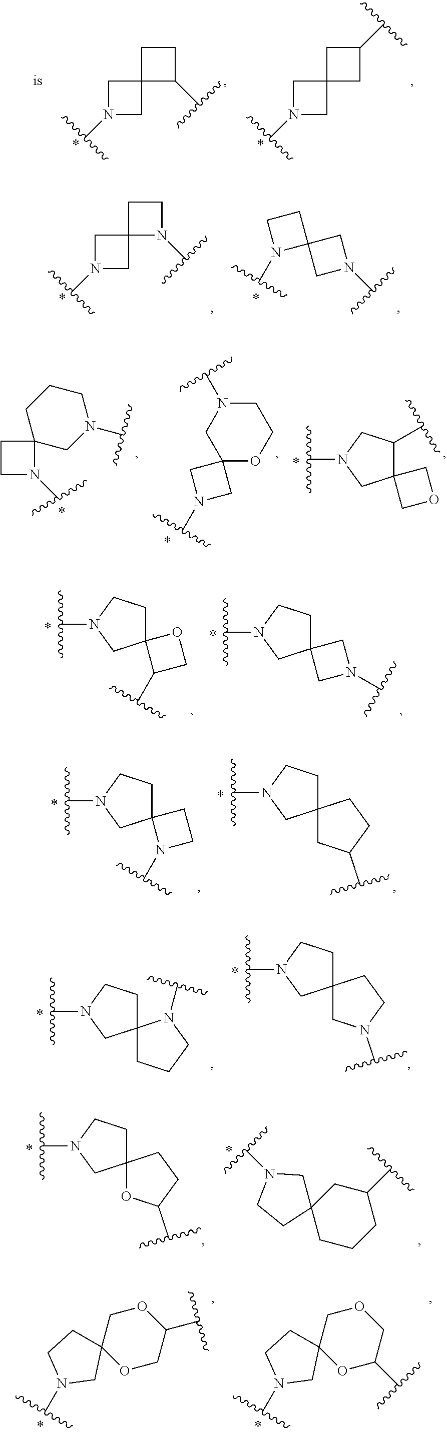

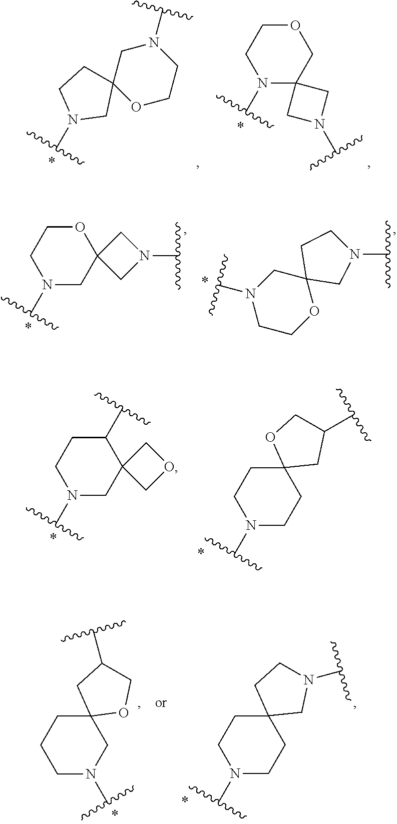

where * indicates connectivity to the carbonyl group at triazolopyridine; and ring B is C₃-C₆ cycloalkyl, 4- to 7-membered heterocyclyl, phenyl, or 5- to 6-membered heteroaryl. In some embodiments, ring A is

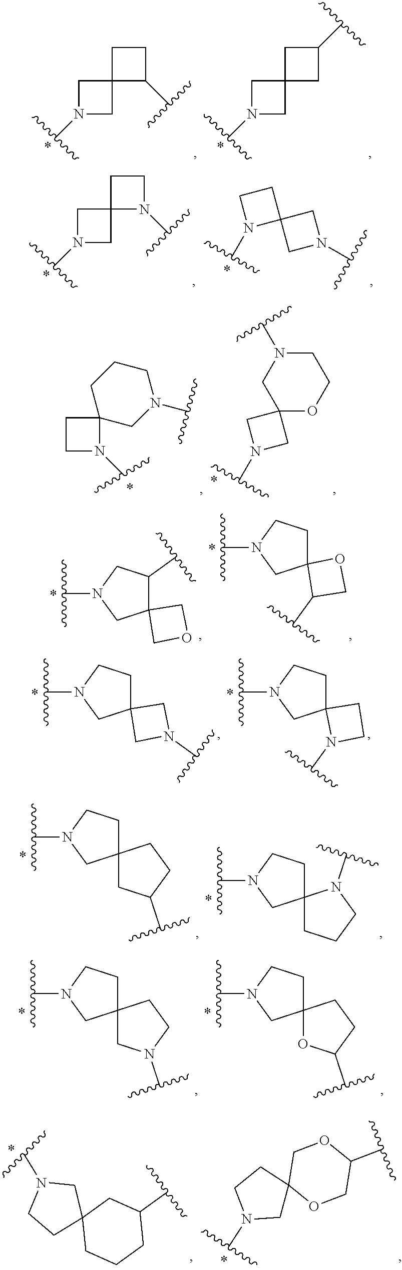

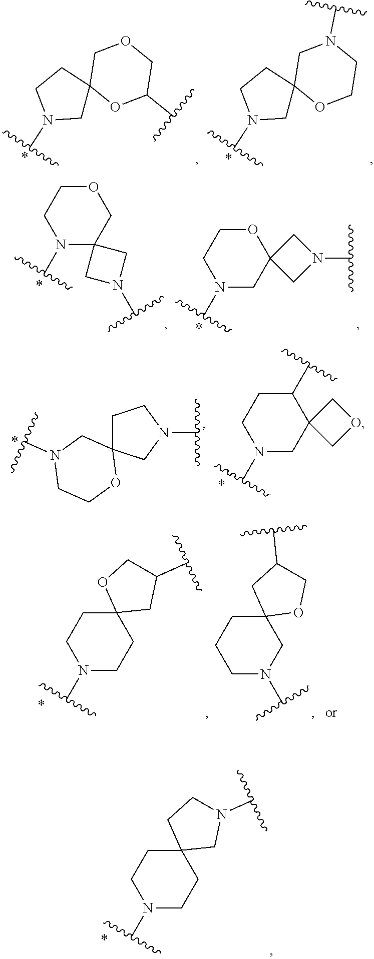

where * indicates connectivity to the carbonyl group at triazolopyridine; and ring B is pyridinyl, pyranyl, thiopyranyl, pyrimidinyl, pyridazinyl, or pyrazinyl. In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments of the compound of Formula (VIII), (VIII-a), or (VIII-b), or a pharmaceutically acceptable salt thereof, there is no (zero) R^{Ta} and no (zero) R^{Tb1}. In some embodiments, there is no (zero) R^{Ta} and one R^{Tb1}. In some embodiments, there is no (zero) R^{Ta} and two R^{Tb1}. In some embodiments, there is no (zero) R^{Ta} and three R^{Tb1}. In some embodiments, there is no (zero) R^{Tb1} and one R^{Ta}. In some embodiments, there is no (zero) R^{Tb1} and two R^{Ta}. In some embodiments, there is no (zero) R^{Tb1} and three R^{Ta}. In some embodiments, each R^{Ta} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—(C₁-C₆ alkyl), —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, —S(═O)₂—(C₁-C₆ alkyl), —S(═O)(═NH)—(C₁-C₆ alkyl), C₁-C₆ alkyl, C₁-C₆ haloalkyl, C₂-C₆ alkenyl, C₂-C₆ haloalkenyl, C₂-C₆ alkynyl, or C₂-C₆ alkynyl. In some embodiments, at least one R^{Tb1} is present and the at least one R^{Tb1} is —C(═O)—H, —C(═O)—OH, or —C(═O)—O(C₁-C₆ alkyl). In some embodiments, at least one R^{Tb1} is —C(═O)—OH.

In some embodiments of (I′), (I″-ii), (I′″-ii), (II′), (II′-a), (II′-b), (II′-c), (II′-d), (II′-e), (II′-f), (III′), (III′-a), (III′-b), (III′-c), (III′-d), (III′-e), (III′-f), (IV′), (IV′-a), (IV′-b), (IV′-c), (IV′-d), (IV′-e), or (IV′-f), U¹ is —O— and U² is —C(R^{U})₂—; R^{U} is H or C₁-C₆ alkyl; and E is methylene optionally substituted with one or more —NH₂. In some embodiments, U² is —O— and U¹—C(R^{U})₂—; R^{U} is H or C₁-C₆ alkyl; and E is methylene optionally substituted with one or more —NH₂. In some embodiments, R^{V} is H, C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Va}. In some embodiments, at least one R^{V} is C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl), wherein the C₁-C₆ alkyl, C₂-C₆ alkenyl, C₂-C₆ alkynyl, C₃-C₈ cycloalkyl, 4- to 10-membered heterocyclyl, C₆-C₁₀ aryl, 5- to 10-membered heteroaryl, —(C₁-C₆ alkyl)-(C₃-C₆ cycloalkyl), —(C₁-C₆ alkyl)-(4- to 10-membered heterocyclyl), —(C₁-C₆ alkyl)-(C₆-C₁₀ aryl), or —(C₁-C₆ alkyl)-(5- to 10-membered heteroaryl) is optionally substituted with one or more R^{Va}. In some embodiments, at least one R^{V} is C₁-C₆ alkyl, C₂-C₆ alkenyl, C₂-C₆ alkynyl, monocyclic C₃-C₈ cycloalkyl, fused bicyclic C₄-C₈ cycloalkyl, bridged bicyclic C₅-C₈ cycloalkyl, spiro bicyclic C₅-C₈ cycloalkyl, monocyclic 4- to 8-membered heterocyclyl, fused bicyclic 4- to 10-membered heterocyclyl, bridged bicyclic 5- to 10-membered heterocyclyl, spiro bicyclic 5- to 10-membered heterocyclyl, phenyl, monocyclic 5- to 6-membered heteroaryl, fused bicyclic 5- to 10-membered heteroaryl, —(C₁-C₄ alkyl)-(monocyclic C₃-C₈ cycloalkyl), —(C₁-C₄ alkyl)-(fused bicyclic C₄-C₈ cycloalkyl), —(C₁-C₄ alkyl)-(bridged bicyclic C₅-C₈ cycloalkyl), —(C₁-C₄ alkyl)-(spiro bicyclic C₅-C₈ cycloalkyl), —(C₁-C₄ alkyl)-(monocyclic 4- to 7-membered heterocyclyl), —(C₁-C₄ alkyl)-(fused bicyclic 4- to 10-membered heterocyclyl), —(C₁-C₄ alkyl)-(bridged bicyclic 5- to 10-membered heterocyclyl), —(C₁-C₄ alkyl)-(spiro bicyclic 5- to 10-membered heterocyclyl), —(C₁-C₄ alkyl)-(phenyl), —(C₁-C₄ alkyl)-(monocyclic 5- to 6-membered heteroaryl), or —(C₁-C₄ alkyl)-(fused bicyclic 5- to 10-membered heteroaryl), each optionally substituted with one or more R^{Va}. In some embodiments, at least one R^{V} is C₁-C₆ alkyl, C₂-C₆ alkenyl, C₂-C₆ alkynyl, cyclopropyl, cyclobutyl, cyclopentyl, cyclohexyl, cycloheptyl, cyclooctyl,

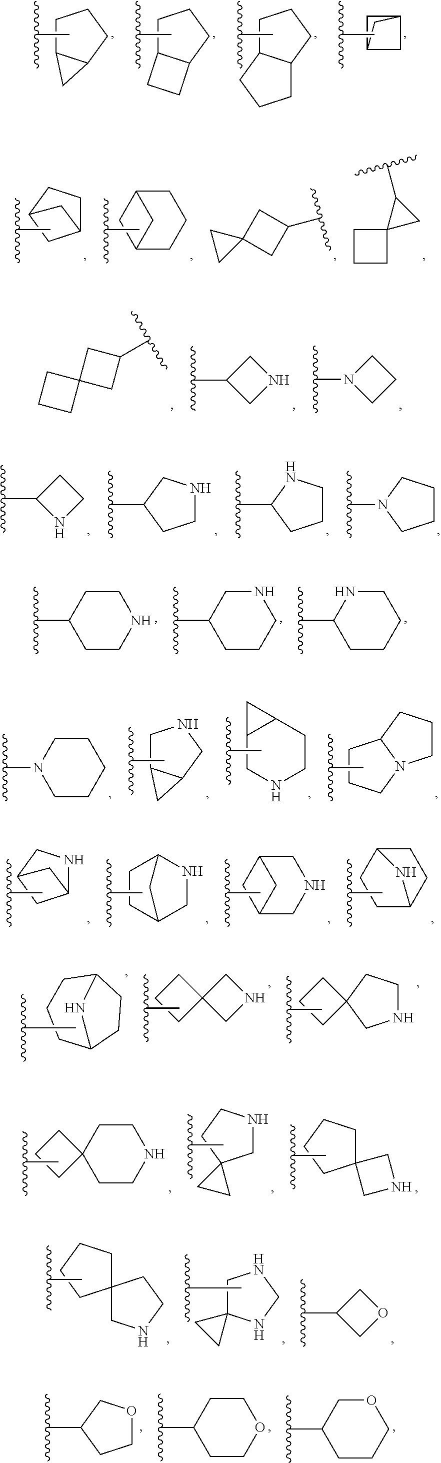

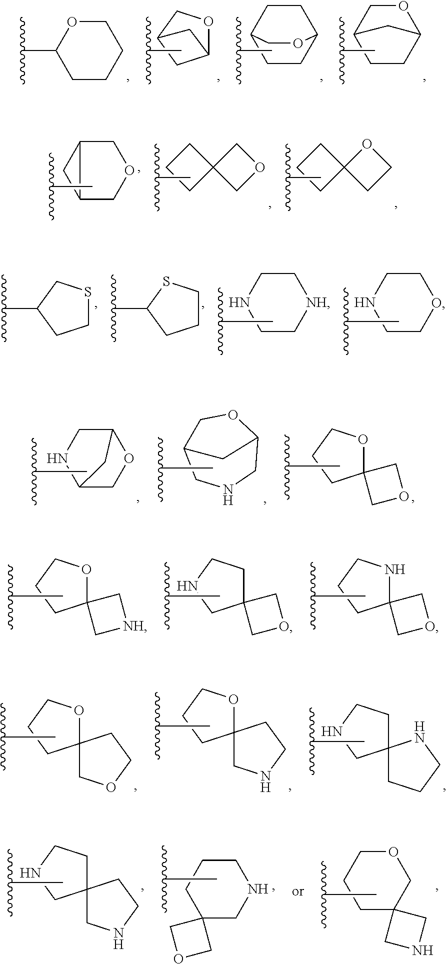

each optionally substituted with one or more R^{Va}. In some embodiments, at least one R^{V} is pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, pyrazinyl, phenyl, or

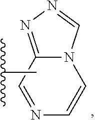

each optionally substituted with one or more R^{Va}.

In some embodiments of (I′), (I″-ii), (I′″-ii), (II′), (II′-a), (II′-b), (II′-c), (II′-d), (II′-e), (II′-f), (III′), (III′-a), (III′-b), (III′-c), (III′-d), (III′-e), (III′-f), (IV′), (IV′-a), (IV′-b), (IV′-c), (IV′-d), (IV′-e), or (IV′-f), each R^{Va} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl; and each R^{Vb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl.

In some embodiments, the compound is selected from the compounds described in Tables 1-5, and pharmaceutically acceptable salts thereof, racemic form thereof, or stereoisomer thereof.

In some embodiments, the compound is selected from the compounds described in Tables 1-5, and pharmaceutically acceptable salts thereof.

In some embodiments, the compound is selected from the compounds described in Tables 1-5.

In some embodiments, the compound is selected from the compounds described in Table 1, and pharmaceutically acceptable salts thereof.

In some embodiments, the compound is selected from the compounds described in Table 1.

In some embodiments, the compound is selected from the compounds described in Table 2, and pharmaceutically acceptable salts thereof.

In some embodiments, the compound is selected from the compounds described in Table 2.

In some embodiments, the compound is selected from the compounds described in Table 3, and pharmaceutically acceptable salts thereof.

In some embodiments, the compound is selected from the compounds described in Table 3.

In some embodiments, the compound is selected from the compounds described in Table 4, and pharmaceutically acceptable salts thereof.

In some embodiments, the compound is selected from the compounds described in Table 4.

In some embodiments, the compound is selected from the compounds described in Table 5, and pharmaceutically acceptable salts thereof.

In some embodiments, the compound is selected from the compounds described in Table 5.

TABLE 1

| Cmpd | |
|---|---|
| # | Structure |
| 1B | 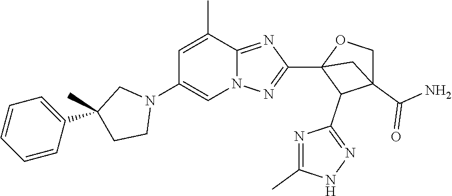 [SMILES: Cc1nc(C2C3(C(N)=O)COC2(c2nc4c(C)cc(N5CC[C@](C)(c6ccccc6)C5)cn4n2)C3)n[nH]1] |
| 41 | 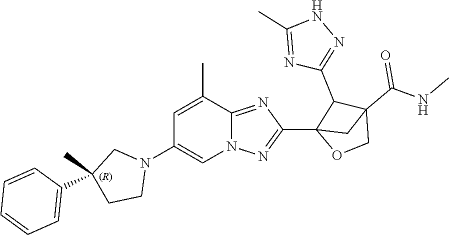 |
| 42 | 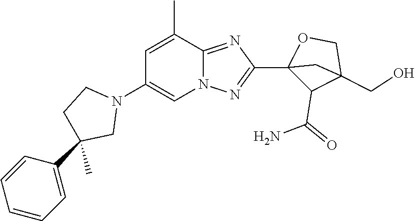 [SMILES: Cc1cc(N2CC[C@](C)(c3ccccc3)C2)cn2nc(C34CC(CO)(CO3)C4C(N)=O)nc12] |
| 43 | 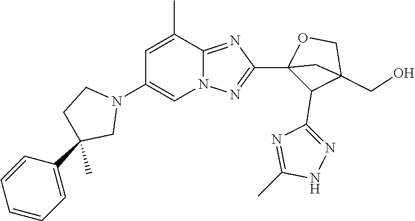 [SMILES: Cc1nc(C2C3(CO)COC2(c2nc4c(C)cc(N5CC[C@](C)(c6ccccc6)C5)cn4n2)C3)n[nH]1] |
| 44 | 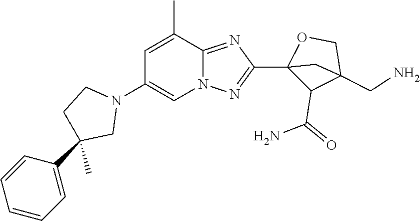 [SMILES: Cc1cc(N2CC[C@](C)(c3ccccc3)C2)cn2nc(C34CC(CN)(CO3)C4C(N)=O)nc12] |
| 45 | 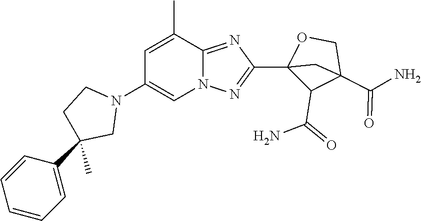 [SMILES: Cc1cc(N2CC[C@](C)(c3ccccc3)C2)cn2nc(C34CC(C(N)=O)(CO3)C4C(N)=O)nc12] |
| 46 | 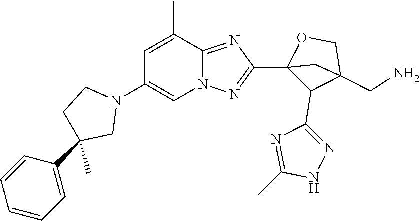 [SMILES: Cc1nc(C2C3(CN)COC2(c2nc4c(C)cc(N5CC[C@](C)(c6ccccc6)C5)cn4n2)C3)n[nH]1] |
| 48 | 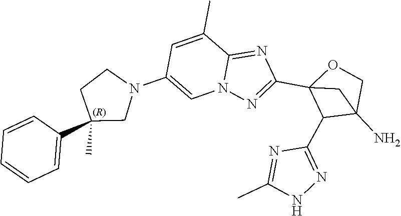 |
| 3 | 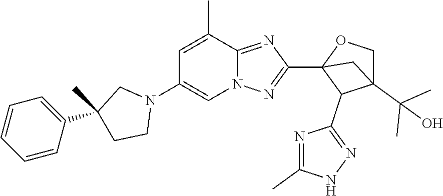 [SMILES: Cc1nc(C2C3(c4nc5c(C)cc(N6CC[C@](C)(c7ccccc7)C6)cn5n4)CC2(C(C)(C)O)CO3)n[nH]1] |
| 4 | 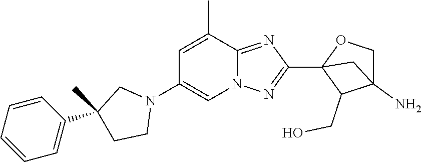 [SMILES: Cc1cc(N2CC[C@](C)(c3ccccc3)C2)cn2nc(C34CC(N)(CO3)C4CO)nc12] |
| 49 | 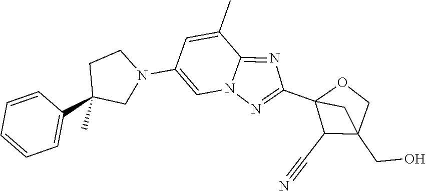 [SMILES: Cc1cc(N2CC[C@](C)(c3ccccc3)C2)cn2nc(C34CC(CO)(CO3)C4C#N)nc12] |
| 50 | 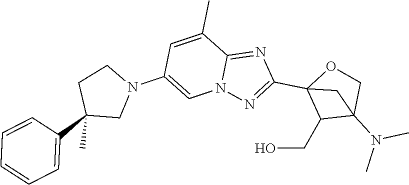 [SMILES: Cc1cc(N2CC[C@](C)(c3ccccc3)C2)cn2nc(C34CC(N(C)C)(CO3)C4CO)nc12] |
| 5 | 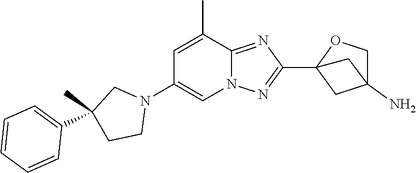 [SMILES: Cc1cc(N2CC[C@](C)(c3ccccc3)C2)cn2nc(C34CC(N)(CO3)C4)nc12] |
| 51 | 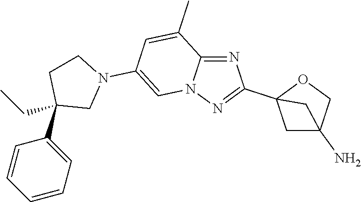 [SMILES: CC[C@]1(c2ccccc2)CCN(c2cc(C)c3nc(C45CC(N)(CO4)C5)nn3c2)C1] |
| 52 | 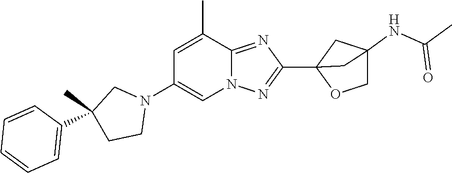 [SMILES: CC(=O)NC12COC(c3nc4c(C)cc(N5CC[C@](C)(c6ccccc6)C5)cn4n3)(C1)C2] |
| 53 | 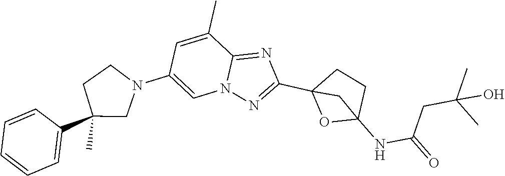 [SMILES: Cc1cc(N2CC[C@](C)(c3ccccc3)C2)cn2nc(C34CCC(NC(=O)CC(C)(C)O)(C3)O4)nc12] |
| 54 | 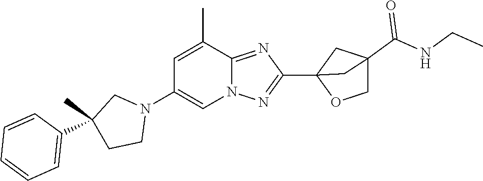 [SMILES: CCNC(=O)C12COC(c3nc4c(C)cc(N5CC[C@](C)(c6ccccc6)C5)cn4n3)(C1)C2] |
| 55 | 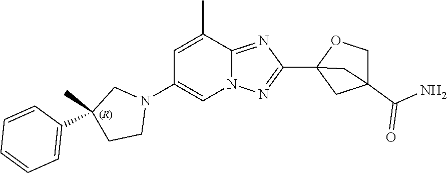 |
| 56 | 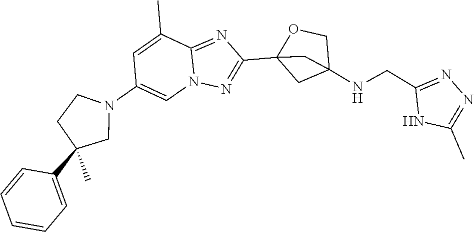 [SMILES: Cc1nnc(CNC23COC(c4nc5c(C)cc(N6CC[C@](C)(c7ccccc7)C6)cn5n4)(C2)C3)[nH]1] |
| 58 | 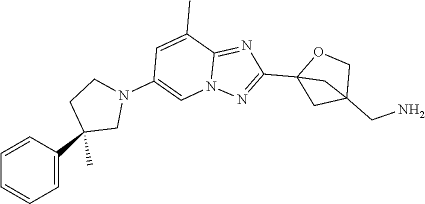 [SMILES: Cc1cc(N2CC[C@](C)(c3ccccc3)C2)cn2nc(C34CC(CN)(CO3)C4)nc12] |
| 59 | 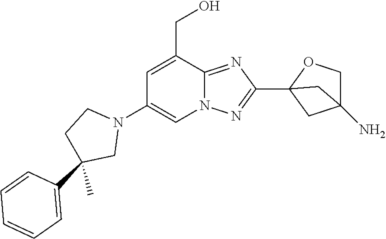 [SMILES: C[C@]1(c2ccccc2)CCN(c2cc(CO)c3nc(C45CC(N)(CO4)C5)nn3c2)C1] |
| 60 | 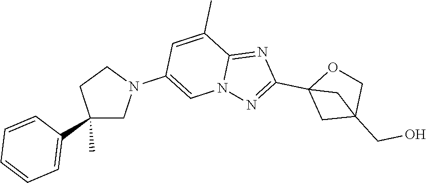 [SMILES: Cc1cc(N2CC[C@](C)(c3ccccc3)C2)cn2nc(C34CC(CO)(CO3)C4)nc12] |
| 62 | 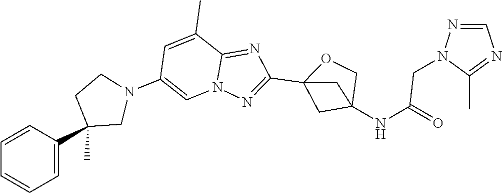 [SMILES: Cc1cc(N2CC[C@](C)(c3ccccc3)C2)cn2nc(C34CC(NC(=O)Cn5ncnc5C)(CO3)C4)nc12] |
| 63 | 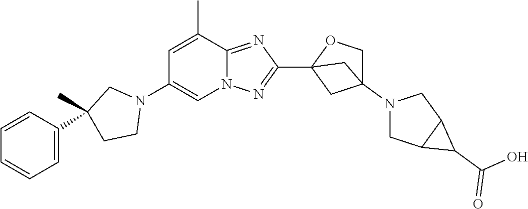 [SMILES: Cc1cc(N2CC[C@](C)(c3ccccc3)C2)cn2nc(C34CC(N5CC6C(C5)C6C(=O)O)(CO3)C4)nc12] |
| 65 | 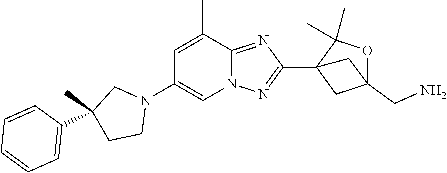 [SMILES: Cc1cc(N2CC[C@](C)(c3ccccc3)C2)cn2nc(C34CC(CN)(C3)OC4(C)C)nc12] |
| 66 | 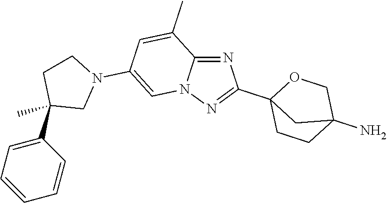 [SMILES: Cc1cc(N2CC[C@](C)(c3ccccc3)C2)cn2nc(C34CCC(N)(CO3)C4)nc12] |
| 67 | 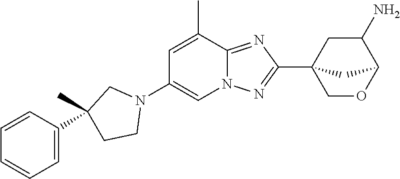 [SMILES: Cc1cc(N2CC[C@](C)(c3ccccc3)C2)cn2nc([C@@]34CO[C@@H](C3)C(N)C4)nc12] |
| 68 | 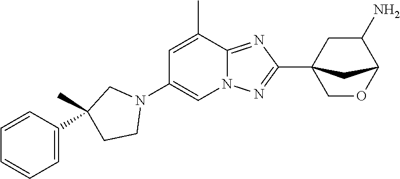 [SMILES: Cc1cc(N2CC[C@](C)(c3ccccc3)C2)cn2nc([C@]34CO[C@H](C3)C(N)C4)nc12] |
| 6 | 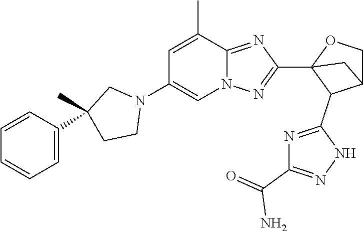 [SMILES: Cc1cc(N2CC[C@](C)(c3ccccc3)C2)cn2nc(C34CC(CO3)C4c3nc(C(N)=O)n[nH]3)nc12] |
| 69 | 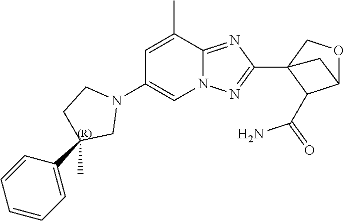 |
| 70 | 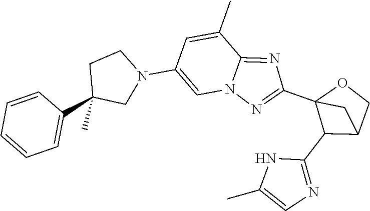 [SMILES: Cc1cnc(C2C3COC2(c2nc4c(C)cc(N5CC[C@](C)(c6ccccc6)C5)cn4n2)C3)[nH]1] |
| 71 | 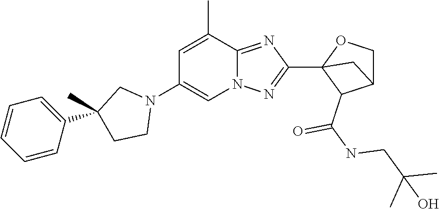 [SMILES: Cc1cc(N2CC[C@](C)(c3ccccc3)C2)cn2nc(C34CC(CO3)C4C(=O)NCC(C)(C)O)nc12] |
| 72 | 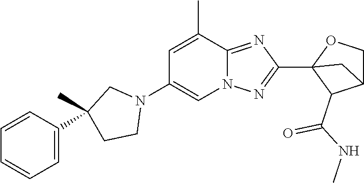 [SMILES: CNC(=O)C1C2COC1(c1nc3c(C)cc(N4CC[C@](C)(c5ccccc5)C4)cn3n1)C2] |
| 73 | 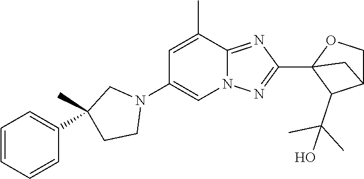 [SMILES: Cc1cc(N2CC[C@](C)(c3ccccc3)C2)cn2nc(C34CC(CO3)C4C(C)(C)O)nc12] |
| 74 | 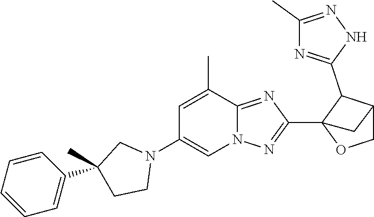 [SMILES: Cc1n[nH]c(C2C3COC2(c2nc4c(C)cc(N5CC[C@](C)(c6ccccc6)C5)cn4n2)C3)n1] |
| 75 | 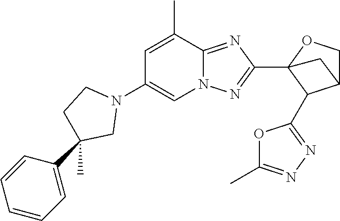 [SMILES: Cc1nnc(C2C3COC2(c2nc4c(C)cc(N5CC[C@](C)(c6ccccc6)C5)cn4n2)C3)o1] |
| 76 | 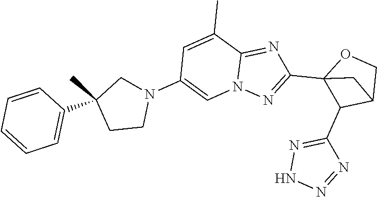 [SMILES: Cc1cc(N2CC[C@](C)(c3ccccc3)C2)cn2nc(C34CC(CO3)C4c3nn[nH]n3)nc12] |
| 77 | 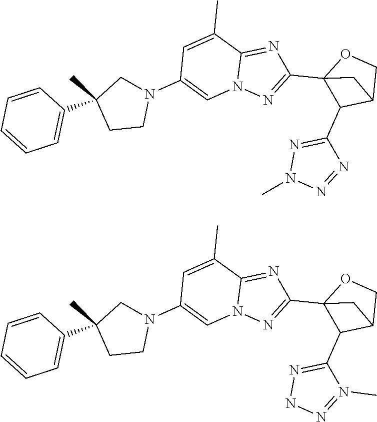 |
| 7 | 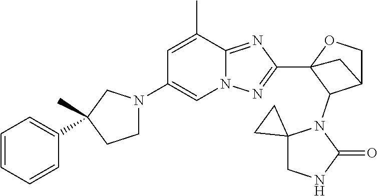 [SMILES: Cc1cc(N2CC[C@](C)(c3ccccc3)C2)cn2nc(C34CC(CO3)C4N3C(=O)NCC34CC4)nc12] |
| 78 | 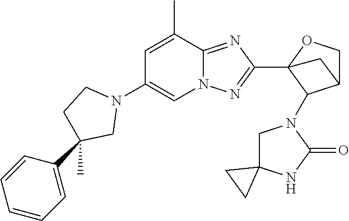 [SMILES: Cc1cc(N2CC[C@](C)(c3ccccc3)C2)cn2nc(C34CC(CO3)C4N3CC4(CC4)NC3=O)nc12] |
| 79 | 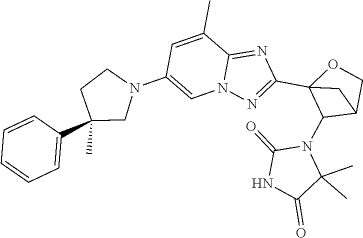 [SMILES: Cc1cc(N2CC[C@](C)(c3ccccc3)C2)cn2nc(C34CC(CO3)C4N3C(=O)NC(=O)C3(C)C)nc12] |
| 8 | 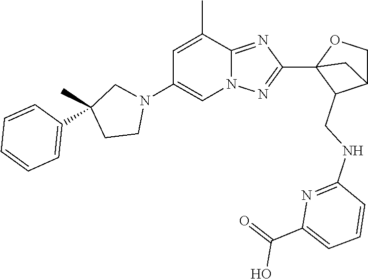 [SMILES: Cc1cc(N2CC[C@](C)(c3ccccc3)C2)cn2nc(C34CC(CO3)C4CNc3cccc(C(=O)O)n3)nc12] |
| 80 | 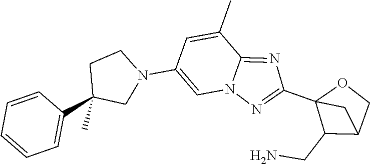 [SMILES: Cc1cc(N2CC[C@](C)(c3ccccc3)C2)cn2nc(C34CC(CO3)C4CN)nc12] |
| 81 | 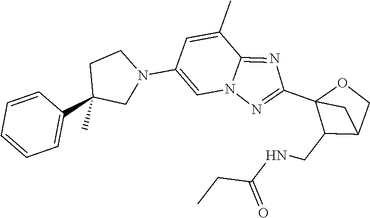 [SMILES: CCC(=O)NCC1C2COC1(c1nc3c(C)cc(N4CC[C@](C)(c5ccccc5)C4)cn3n1)C2] |
| 9 | 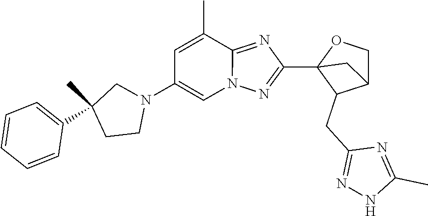 [SMILES: Cc1nc(CC2C3COC2(c2nc4c(C)cc(N5CC[C@](C)(c6ccccc6)C5)cn4n2)C3)n[nH]1] |
| 82 | 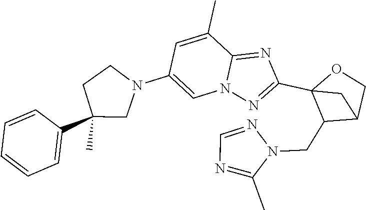 [SMILES: Cc1cc(N2CC[C@](C)(c3ccccc3)C2)cn2nc(C34CC(CO3)C4Cn3ncnc3C)nc12] |
| 83 | 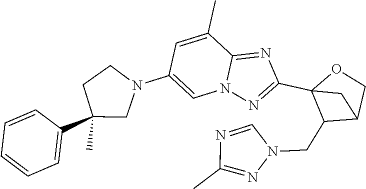 [SMILES: Cc1ncn(CC2C3COC2(c2nc4c(C)cc(N5CC[C@](C)(c6ccccc6)C5)cn4n2)C3)n1] |
| 84 | 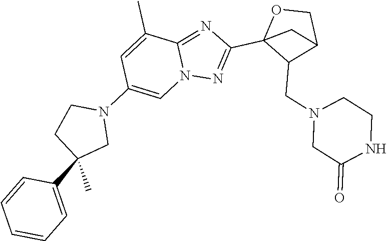 [SMILES: Cc1cc(N2CC[C@](C)(c3ccccc3)C2)cn2nc(C34CC(CO3)C4CN3CCNC(=O)C3)nc12] |
| 85 | 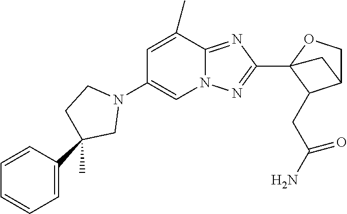 [SMILES: Cc1cc(N2CC[C@](C)(c3ccccc3)C2)cn2nc(C34CC(CO3)C4CC(N)=O)nc12] |
| 86 | 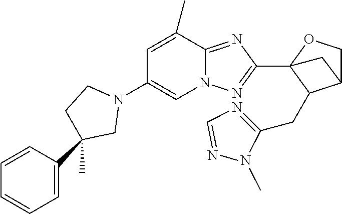 [SMILES: Cc1cc(N2CC[C@](C)(c3ccccc3)C2)cn2nc(C34CC(CO3)C4Cc3ncnn3C)nc12] |
| 87 | 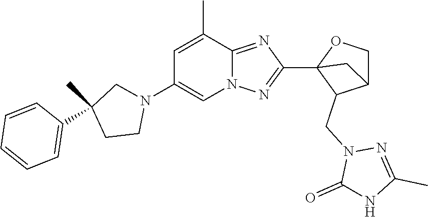 [SMILES: Cc1nn(CC2C3COC2(c2nc4c(C)cc(N5CC[C@](C)(c6ccccc6)C5)cn4n2)C3)c(=O)[nH]1] |
| 88 | 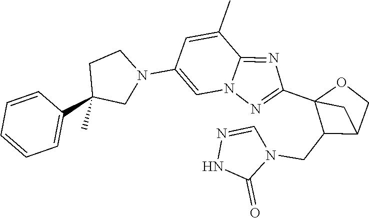 [SMILES: Cc1cc(N2CC[C@](C)(c3ccccc3)C2)cn2nc(C34CC(CO3)C4Cn3cn[nH]c3=O)nc12] |
| 89 | 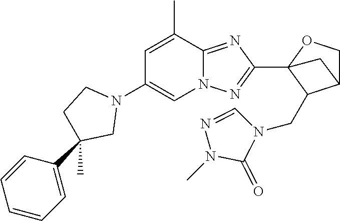 [SMILES: Cc1cc(N2CC[C@](C)(c3ccccc3)C2)cn2nc(C34CC(CO3)C4Cn3cnn(C)c3=O)nc12] |
| 90 | 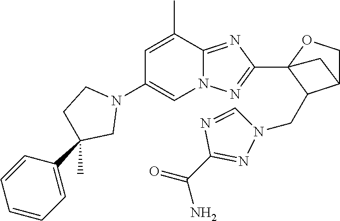 [SMILES: Cc1cc(N2CC[C@](C)(c3ccccc3)C2)cn2nc(C34CC(CO3)C4Cn3cnc(C(N)=O)n3)nc12] |
| 91 | 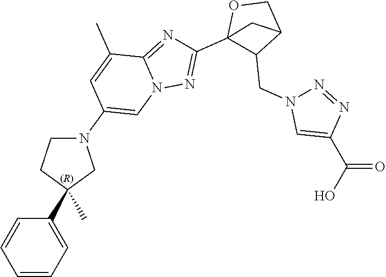 |
| 92 | 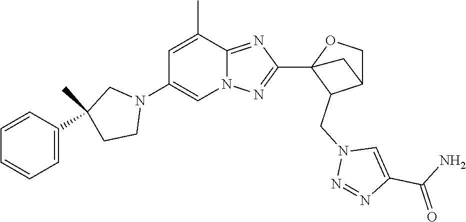 [SMILES: Cc1cc(N2CC[C@](C)(c3ccccc3)C2)cn2nc(C34CC(CO3)C4Cn3cc(C(N)=O)nn3)nc12] |
| 10 | 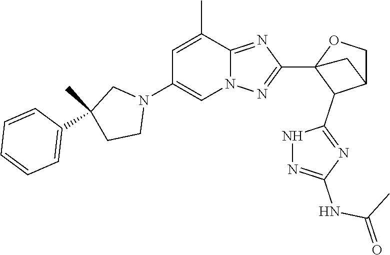 [SMILES: CC(=O)Nc1n[nH]c(C2C3COC2(c2nc4c(C)cc(N5CC[C@](C)(c6ccccc6)C5)cn4n2)C3)n1] |
| 93 | 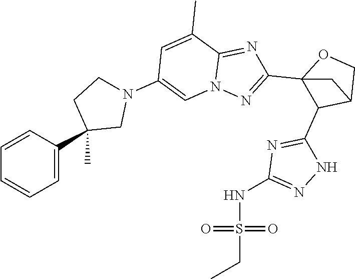 [SMILES: CCS(=O)(=O)Nc1n[nH]c(C2C3COC2(c2nc4c(C)cc(N5CC[C@](C)(c6ccccc6)C5)cn4n2)C3)n1] |
| 11 | 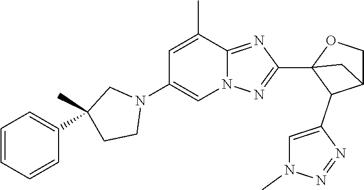 [SMILES: Cc1cc(N2CC[C@](C)(c3ccccc3)C2)cn2nc(C34CC(CO3)C4c3cn(C)nn3)nc12] |
| 13 | 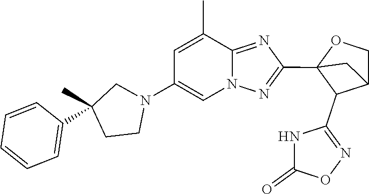 [SMILES: Cc1cc(N2CC[C@](C)(c3ccccc3)C2)cn2nc(C34CC(CO3)C4c3noc(=O)[nH]3)nc12] |
| 97 | 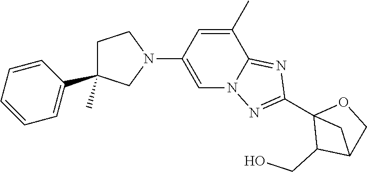 [SMILES: Cc1cc(N2CC[C@](C)(c3ccccc3)C2)cn2nc(C34CC(CO3)C4CO)nc12] |
| 344 | 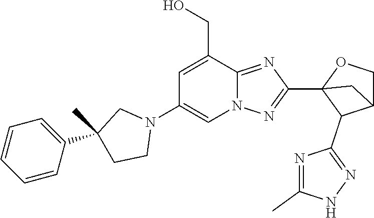 [SMILES: Cc1nc(C2C3COC2(c2nc4c(CO)cc(N5CC[C@](C)(c6ccccc6)C5)cn4n2)C3)n[nH]1] |
| 381 | 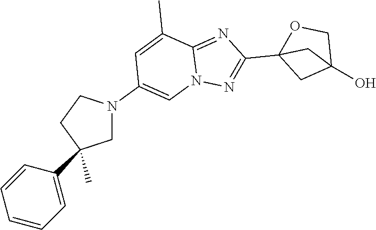 [SMILES: Cc1cc(N2CC[C@](C)(c3ccccc3)C2)cn2nc(C34CC(O)(CO3)C4)nc12] |
| 438 | 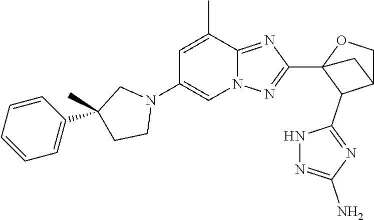 [SMILES: Cc1cc(N2CC[C@](C)(c3ccccc3)C2)cn2nc(C34CC(CO3)C4c3nc(N)n[nH]3)nc12] |
| 440 | 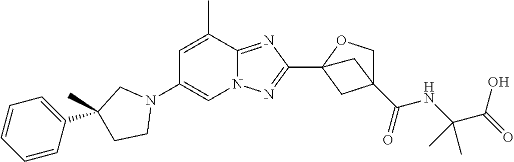 [SMILES: Cc1cc(N2CC[C@](C)(c3ccccc3)C2)cn2nc(C34CC(C(=O)NC(C)(C)C(=O)O)(CO3)C4)nc12] |
| 441 | 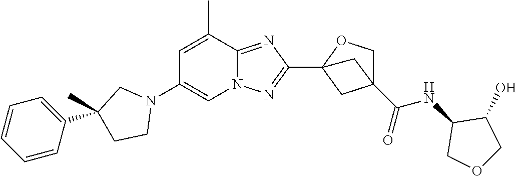 [SMILES: Cc1cc(N2CC[C@](C)(c3ccccc3)C2)cn2nc(C34CC(C(=O)N[C@@H]5COC[C@H]5O)(CO3)C4)nc12] |
| 442 | 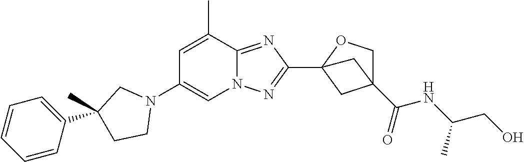 [SMILES: Cc1cc(N2CC[C@](C)(c3ccccc3)C2)cn2nc(C34CC(C(=O)N[C@@H](C)CO)(CO3)C4)nc12] |
| 443 | 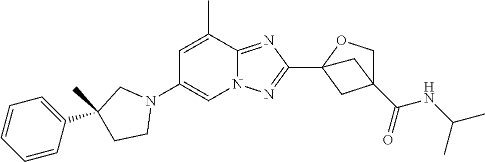 [SMILES: Cc1cc(N2CC[C@](C)(c3ccccc3)C2)cn2nc(C34CC(C(=O)NC(C)C)(CO3)C4)nc12] |
| 444 | 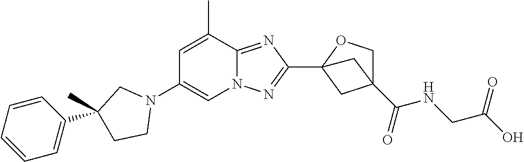 [SMILES: Cc1cc(N2CC[C@](C)(c3ccccc3)C2)cn2nc(C34CC(C(=O)NCC(=O)O)(CO3)C4)nc12] |
| 445 | 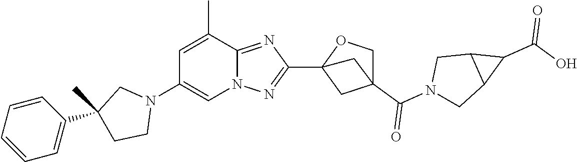 [SMILES: Cc1cc(N2CC[C@](C)(c3ccccc3)C2)cn2nc(C34CC(C(=O)N5CC6C(C5)C6C(=O)O)(CO3)C4)nc12] |
| 446 | 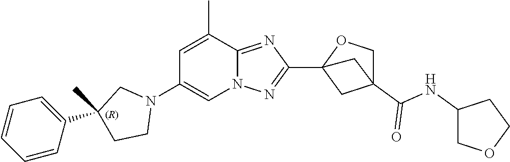 |
| 447 | 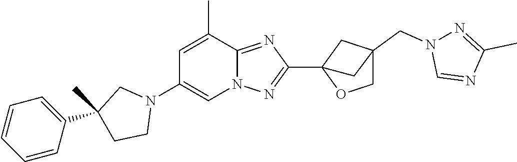 [SMILES: Cc1ncn(CC23COC(c4nc5c(C)cc(N6CC[C@](C)(c7ccccc7)C6)cn5n4)(C2)C3)n1] |
| 448 | 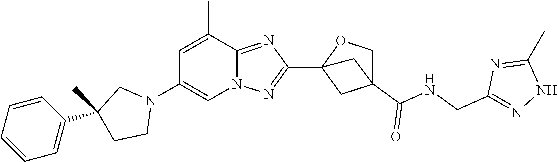 [SMILES: Cc1nc(CNC(=O)C23COC(c4nc5c(C)cc(N6CC[C@](C)(c7ccccc7)C6)cn5n4)(C2)C3)n[nH]1] |
| 449 | 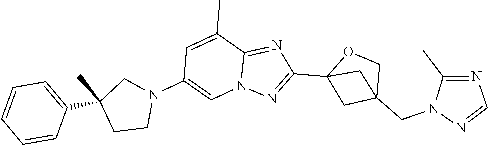 [SMILES: Cc1cc(N2CC[C@](C)(c3ccccc3)C2)cn2nc(C34CC(Cn5ncnc5C)(CO3)C4)nc12] |
| 450 | 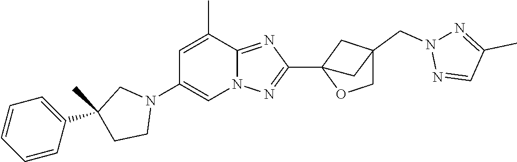 [SMILES: Cc1cnn(CC23COC(c4nc5c(C)cc(N6CC[C@](C)(c7ccccc7)C6)cn5n4)(C2)C3)n1] |
| 451 | 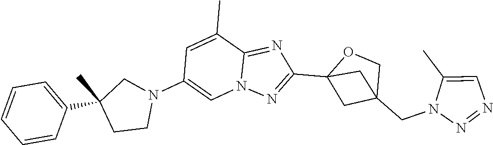 [SMILES: Cc1cc(N2CC[C@](C)(c3ccccc3)C2)cn2nc(C34CC(Cn5nncc5C)(CO3)C4)nc12] |
| 452 | 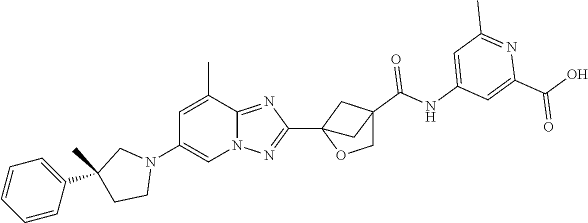 [SMILES: Cc1cc(NC(=O)C23COC(c4nc5c(C)cc(N6CC[C@](C)(c7ccccc7)C6)cn5n4)(C2)C3)cc(C(=O)O)n1] |
| 453 | 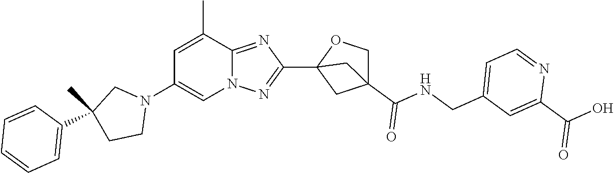 [SMILES: Cc1cc(N2CC[C@](C)(c3ccccc3)C2)cn2nc(C34CC(C(=O)NCc5ccnc(C(=O)O)c5)(CO3)C4)nc12] |
| 454 | 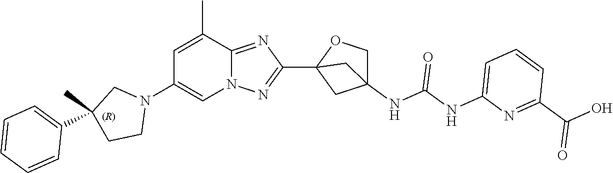 |
| 455 | 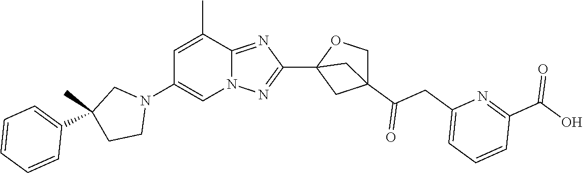 [SMILES: Cc1cc(N2CC[C@](C)(c3ccccc3)C2)cn2nc(C34CC(C(=O)Cc5cccc(C(=O)O)n5)(CO3)C4)nc12] |
| 456 | 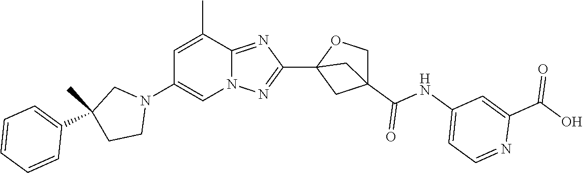 [SMILES: Cc1cc(N2CC[C@](C)(c3ccccc3)C2)cn2nc(C34CC(C(=O)Nc5ccnc(C(=O)O)c5)(CO3)C4)nc12] |
| 457 | 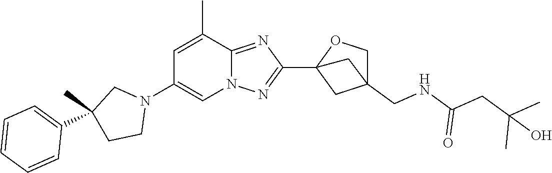 [SMILES: Cc1cc(N2CC[C@](C)(c3ccccc3)C2)cn2nc(C34CC(CNC(=O)CC(C)(C)O)(CO3)C4)nc12] |
| 458 | 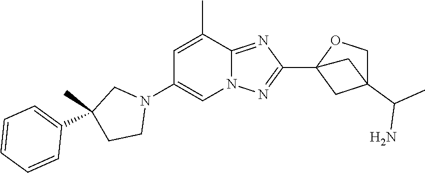 [SMILES: Cc1cc(N2CC[C@](C)(c3ccccc3)C2)cn2nc(C34CC(C(C)N)(CO3)C4)nc12] |
| 459 | 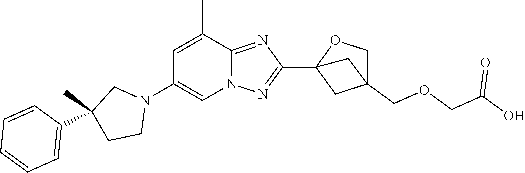 [SMILES: Cc1cc(N2CC[C@](C)(c3ccccc3)C2)cn2nc(C34CC(COCC(=O)O)(CO3)C4)nc12] |
| 460 | 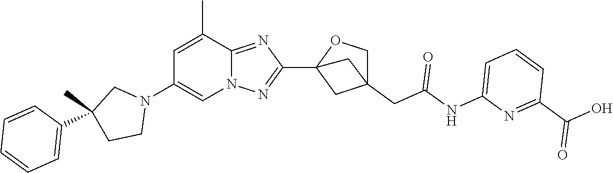 [SMILES: Cc1cc(N2CC[C@](C)(c3ccccc3)C2)cn2nc(C34CC(CC(=O)Nc5cccc(C(=O)O)n5)(CO3)C4)nc12] |
| 461 | 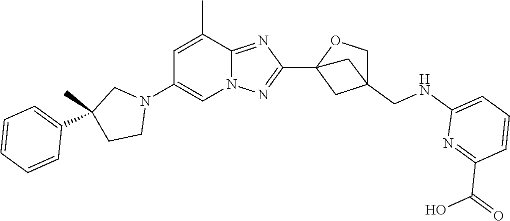 [SMILES: Cc1cc(N2CC[C@](C)(c3ccccc3)C2)cn2nc(C34CC(CNc5cccc(C(=O)O)n5)(CO3)C4)nc12] |
| 462 | 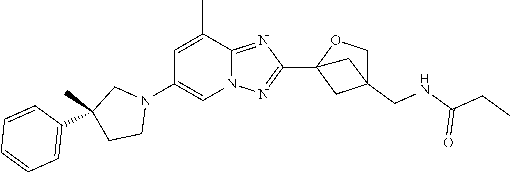 [SMILES: CCC(=O)NCC12COC(c3nc4c(C)cc(N5CC[C@](C)(c6ccccc6)C5)cn4n3)(C1)C2] |
| 463 | 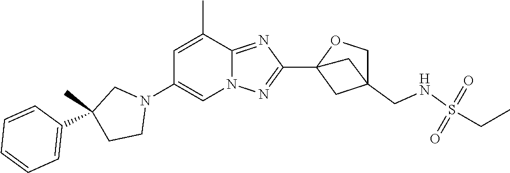 [SMILES: CCS(=O)(=O)NCC12COC(c3nc4c(C)cc(N5CC[C@](C)(c6ccccc6)C5)cn4n3)(C1)C2] |
| 464 | 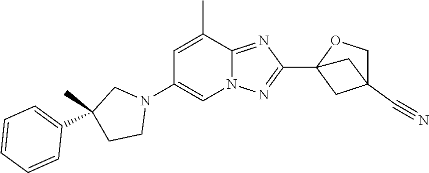 [SMILES: Cc1cc(N2CC[C@](C)(c3ccccc3)C2)cn2nc(C34CC(C#N)(CO3)C4)nc12] |
| 465 | 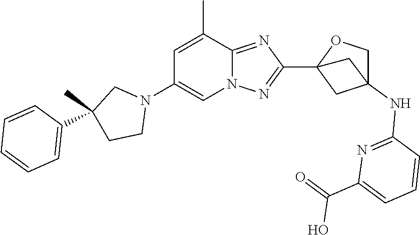 [SMILES: Cc1cc(N2CC[C@](C)(c3ccccc3)C2)cn2nc(C34CC(Nc5cccc(C(=O)O)n5)(CO3)C4)nc12] |
| 466 | 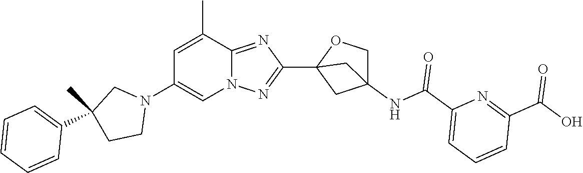 [SMILES: Cc1cc(N2CC[C@](C)(c3ccccc3)C2)cn2nc(C34CC(NC(=O)c5cccc(C(=O)O)n5)(CO3)C4)nc12] |
| 467 | 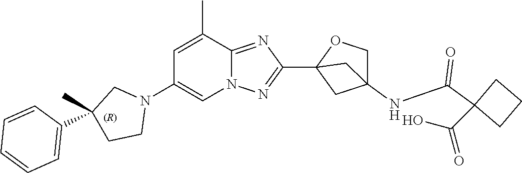 |
| 468 | 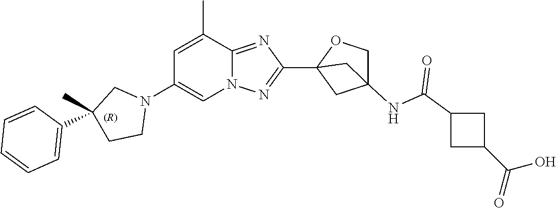 |
| 469 | 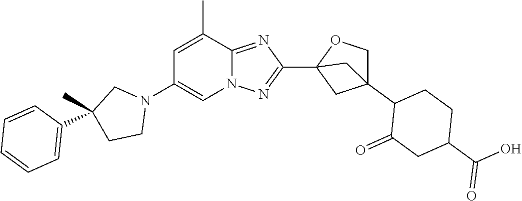 [SMILES: Cc1cc(N2CC[C@](C)(c3ccccc3)C2)cn2nc(C34CC(C5CCC(C(=O)O)CC5=O)(CO3)C4)nc12] |
| 470 | 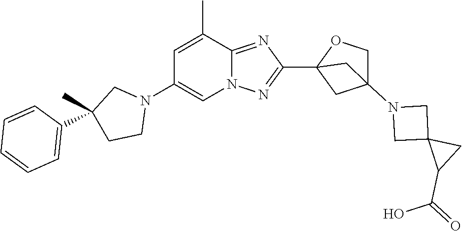 [SMILES: Cc1cc(N2CC[C@](C)(c3ccccc3)C2)cn2nc(C34CC(N5CC6(CC6C(=O)O)C5)(CO3)C4)nc12] |
| 471 | 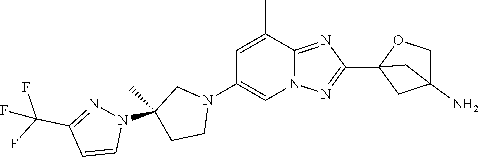 [SMILES: Cc1cc(N2CC[C@@](C)(n3ccc(C(F)(F)F)n3)C2)cn2nc(C34CC(N)(CO3)C4)nc12] |
| 472 | 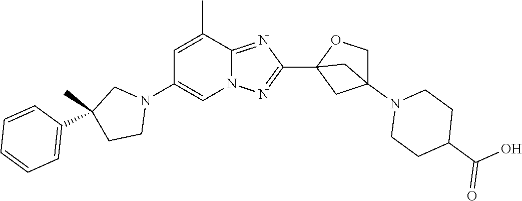 [SMILES: Cc1cc(N2CC[C@](C)(c3ccccc3)C2)cn2nc(C34CC(N5CCC(C(=O)O)CC5)(CO3)C4)nc12] |
| 473 | 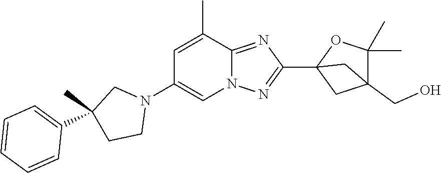 [SMILES: Cc1cc(N2CC[C@](C)(c3ccccc3)C2)cn2nc(C34CC(CO)(C3)C(C)(C)O4)nc12] |
| 477 | 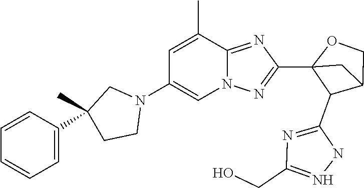 [SMILES: Cc1cc(N2CC[C@](C)(c3ccccc3)C2)cn2nc(C34CC(CO3)C4c3nc(CO)n[nH]3)nc12] |
| 583 | 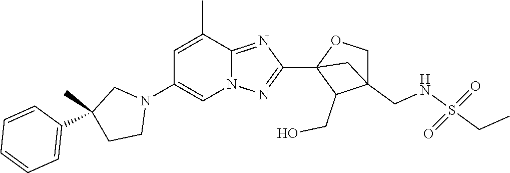 [SMILES: CCS(=O)(=O)NCC12COC(c3nc4c(C)cc(N5CC[C@](C)(c6ccccc6)C5)cn4n3)(C1)C2CO] |
| 584 | 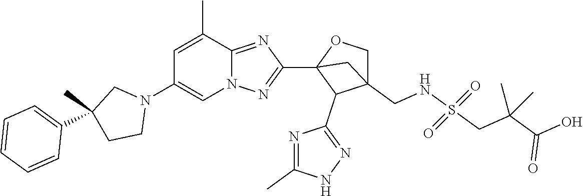 [SMILES: Cc1nc(C2C3(CNS(=O)(=O)CC(C)(C)C(=O)O)COC2(c2nc4c(C)cc(N5CC[C@](C)(c6ccccc6)C5)cn4n2)C3)n[nH]1] |
| 585 | 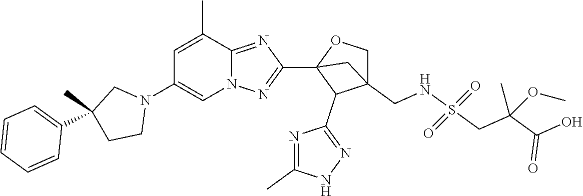 [SMILES: COC(C)(CS(=O)(=O)NCC12COC(c3nc4c(C)cc(N5CC[C@](C)(c6ccccc6)C5)cn4n3)(C1)C2c1n[nH]c(C)n1)C(=O)O] |
| 586 | 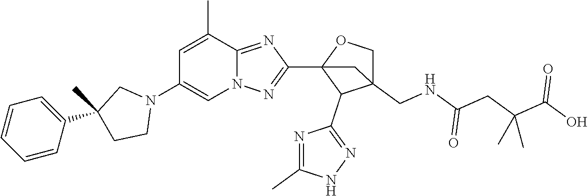 [SMILES: Cc1nc(C2C3(CNC(=O)CC(C)(C)C(=O)O)COC2(c2nc4c(C)cc(N5CC[C@](C)(c6ccccc6)C5)cn4n2)C3)n[nH]1] |
| 478 | 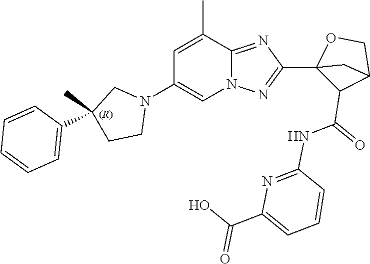 |
| 479 | 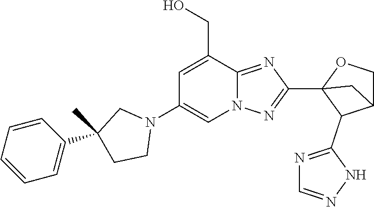 [SMILES: C[C@]1(c2ccccc2)CCN(c2cc(CO)c3nc(C45CC(CO4)C5c4ncn[nH]4)nn3c2)C1] |
| 480 | 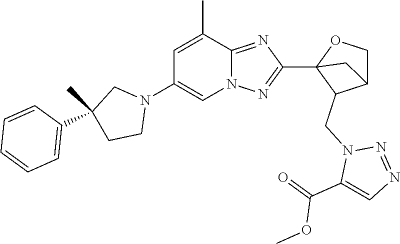 [SMILES: COC(=O)c1cnnn1CC1C2COC1(c1nc3c(C)cc(N4CC[C@](C)(c5ccccc5)C4)cn3n1)C2] |
| 481 | 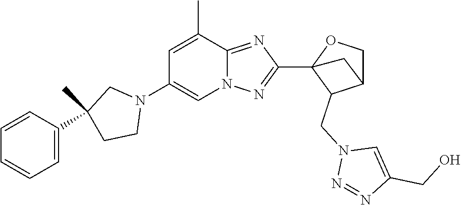 [SMILES: Cc1cc(N2CC[C@](C)(c3ccccc3)C2)cn2nc(C34CC(CO3)C4Cn3cc(CO)nn3)nc12] |
| 482 | 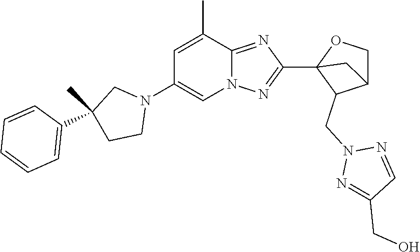 [SMILES: Cc1cc(N2CC[C@](C)(c3ccccc3)C2)cn2nc(C34CC(CO3)C4Cn3ncc(CO)n3)nc12] |
| 483 | 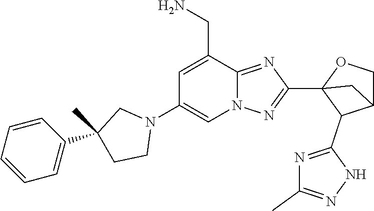 [SMILES: Cc1n[nH]c(C2C3COC2(c2nc4c(CN)cc(N5CC[C@](C)(c6ccccc6)C5)cn4n2)C3)n1] |
| 484 | 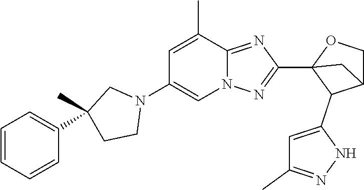 [SMILES: Cc1cc(C2C3COC2(c2nc4c(C)cc(N5CC[C@](C)(c6ccccc6)C5)cn4n2)C3)[nH]n1] |
| 485 | 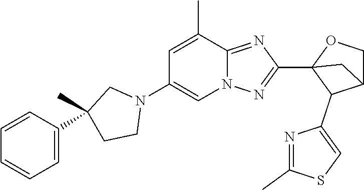 [SMILES: Cc1nc(C2C3COC2(c2nc4c(C)cc(N5CC[C@](C)(c6ccccc6)C5)cn4n2)C3)cs1] |
| 488 | 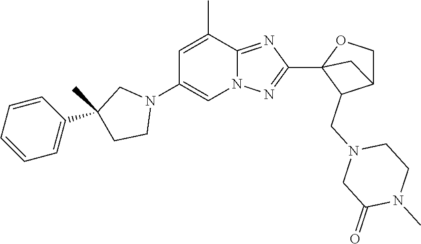 [SMILES: Cc1cc(N2CC[C@](C)(c3ccccc3)C2)cn2nc(C34CC(CO3)C4CN3CCN(C)C(=O)C3)nc12] |
| 489 | 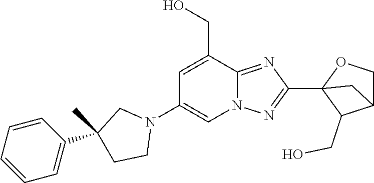 [SMILES: C[C@]1(c2ccccc2)CCN(c2cc(CO)c3nc(C45CC(CO4)C5CO)nn3c2)C1] |
| 490 | 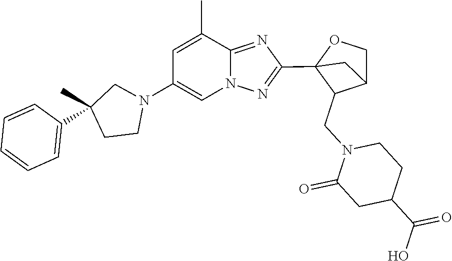 [SMILES: Cc1cc(N2CC[C@](C)(c3ccccc3)C2)cn2nc(C34CC(CO3)C4CN3CCC(C(=O)O)CC3=O)nc12] |
| 491 | 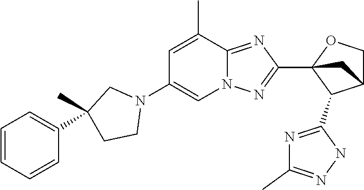 [SMILES: Cc1n[nH]c([C@H]2[C@H]3CO[C@]2(c2nc4c(C)cc(N5CC[C@](C)(c6ccccc6)C5)cn4n2)C3)n1] |
| 492 | 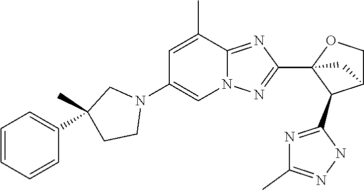 [SMILES: Cc1n[nH]c([C@@H]2[C@@H]3CO[C@@]2(c2nc4c(C)cc(N5CC[C@](C)(c6ccccc6)C5)cn4n2)C3)n1] |
| 493 | 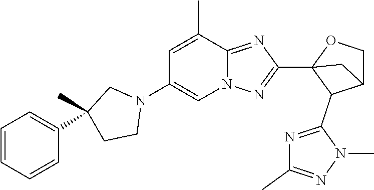 [SMILES: Cc1nc(C2C3COC2(c2nc4c(C)cc(N5CC[C@](C)(c6ccccc6)C5)cn4n2)C3)n(C)n1] |
| 494 | 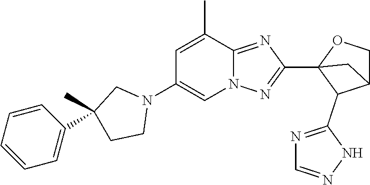 [SMILES: Cc1cc(N2CC[C@](C)(c3ccccc3)C2)cn2nc(C34CC(CO3)C4c3ncn[nH]3)nc12] |
| 495 | 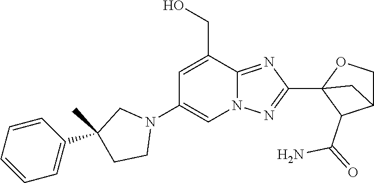 [SMILES: C[C@]1(c2ccccc2)CCN(c2cc(CO)c3nc(C45CC(CO4)C5C(N)=O)nn3c2)C1] |
| 496 | 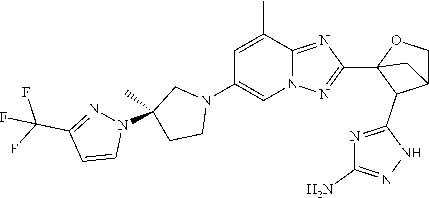 [SMILES: Cc1cc(N2CC[C@@](C)(n3ccc(C(F)(F)F)n3)C2)cn2nc(C34CC(CO3)C4c3nc(N)n[nH]3)nc12] |
| 497 | 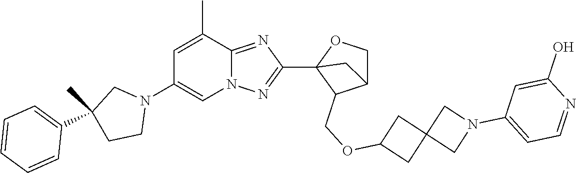 [SMILES: Cc1cc(N2CC[C@](C)(c3ccccc3)C2)cn2nc(C34CC(CO3)C4COC3CC4(C3)CN(c3ccnc(O)c3)C4)nc12] |
| 498 | 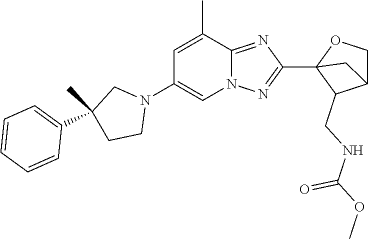 [SMILES: COC(=O)NCC1C2COC1(c1nc3c(C)cc(N4CC[C@](C)(c5ccccc5)C4)cn3n1)C2] |
| 499 | 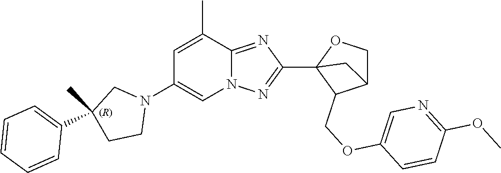 |
| 500 | 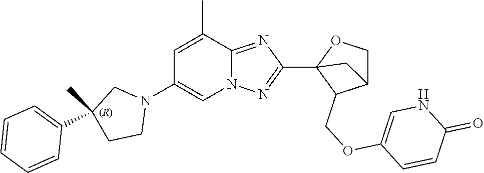 |
| 501 | 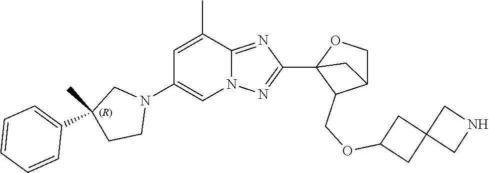 |
| 502 | 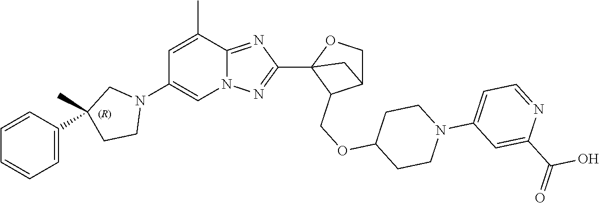 |
| 503 | 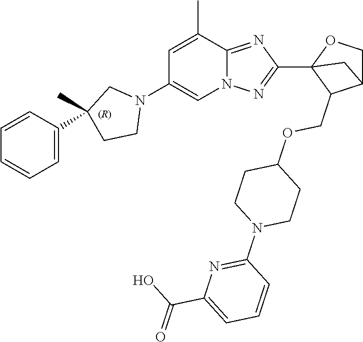 |
| 504 | 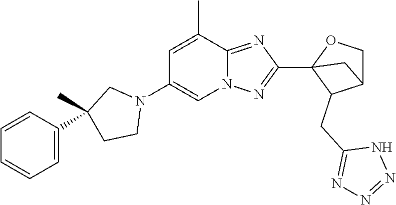 [SMILES: Cc1cc(N2CC[C@](C)(c3ccccc3)C2)cn2nc(C34CC(CO3)C4Cc3nnn[nH]3)nc12] |
| 505 | 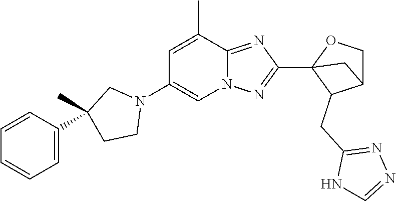 [SMILES: Cc1cc(N2CC[C@](C)(c3ccccc3)C2)cn2nc(C34CC(CO3)C4Cc3nnc[nH]3)nc12] |
| 506 | 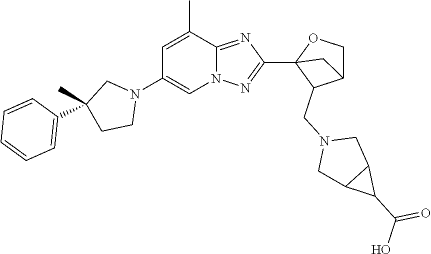 [SMILES: Cc1cc(N2CC[C@](C)(c3ccccc3)C2)cn2nc(C34CC(CO3)C4CN3CC4C(C3)C4C(=O)O)nc12] |
| 507 | 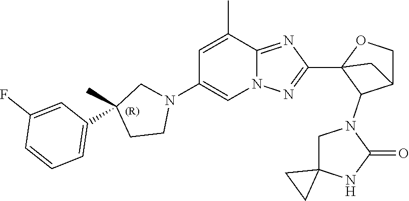 |
| 508 | 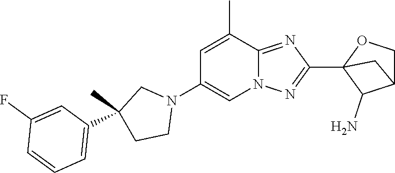 [SMILES: Cc1cc(N2CC[C@](C)(c3cccc(F)c3)C2)cn2nc(C34CC(CO3)C4N)nc12] |
| 509 | 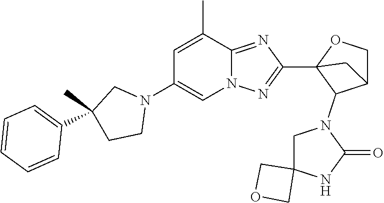 [SMILES: Cc1cc(N2CC[C@](C)(c3ccccc3)C2)cn2nc(C34CC(CO3)C4N3CC4(COC4)NC3=O)nc12] |
| 510 | 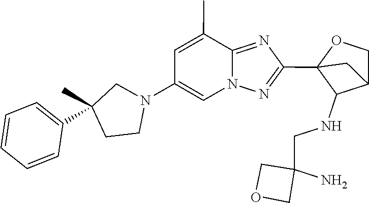 [SMILES: Cc1cc(N2CC[C@](C)(c3ccccc3)C2)cn2nc(C34CC(CO3)C4NCC3(N)COC3)nc12] |
| 511 | 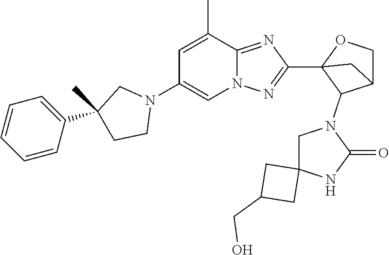 [SMILES: Cc1cc(N2CC[C@](C)(c3ccccc3)C2)cn2nc(C34CC(CO3)C4N3CC4(CC(CO)C4)NC3=O)nc12] |
| 513 | 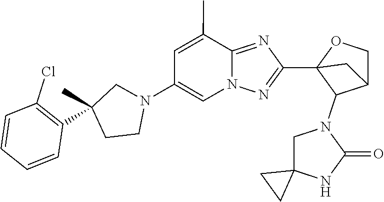 [SMILES: Cc1cc(N2CC[C@](C)(c3ccccc3Cl)C2)cn2nc(C34CC(CO3)C4N3CC4(CC4)NC3=O)nc12] |
| 514 | 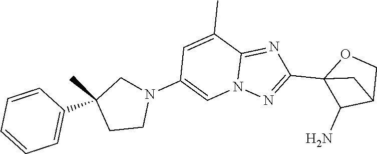 [SMILES: Cc1cc(N2CC[C@](C)(c3ccccc3)C2)cn2nc(C34CC(CO3)C4N)nc12] |
| 515 | 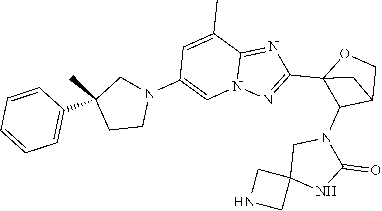 [SMILES: Cc1cc(N2CC[C@](C)(c3ccccc3)C2)cn2nc(C34CC(CO3)C4N3CC4(CNC4)NC3=O)nc12] |
| 516 | 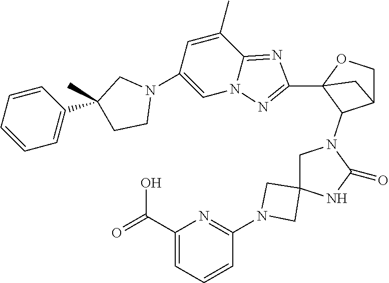 [SMILES: Cc1cc(N2CC[C@](C)(c3ccccc3)C2)cn2nc(C34CC(CO3)C4N3CC4(CN(c5cccc(C(=O)O)n5)C4)NC3=O)nc12] |
| 517 | 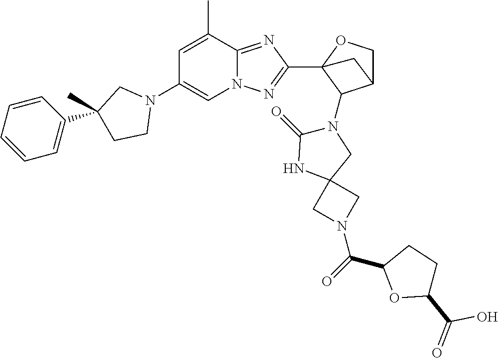 |
| 518 | 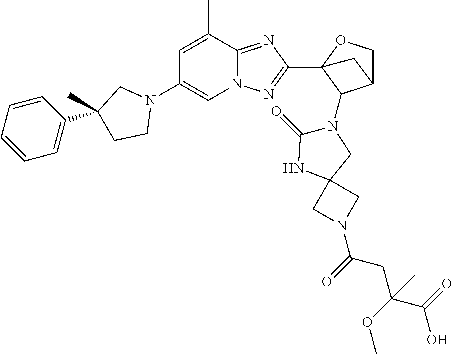 [SMILES: COC(C)(CC(=O)N1CC2(C1)CN(C1C3COC1(c1nc4c(C)cc(N5CC[C@](C)(c6ccccc6)C5)cn4n1)C3)C(=O)N2)C(=O)O] |
| 519 | 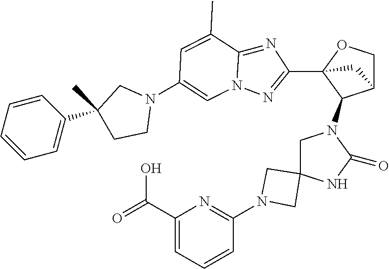 [SMILES: Cc1cc(N2CC[C@](C)(c3ccccc3)C2)cn2nc([C@@]34C[C@@H](CO3)[C@H]4N3CC4(CN(c5cccc(C(=O)O)n5)C4)NC3=O)nc12] |
| 520 | 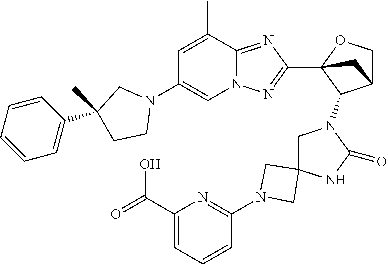 [SMILES: Cc1cc(N2CC[C@](C)(c3ccccc3)C2)cn2nc([C@]34C[C@H](CO3)[C@@H]4N3CC4(CN(c5cccc(C(=O)O)n5)C4)NC3=O)nc12] |
| 521 | 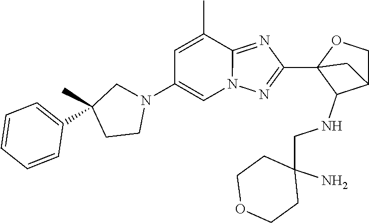 [SMILES: Cc1cc(N2CC[C@](C)(c3ccccc3)C2)cn2nc(C34CC(CO3)C4NCC3(N)CCOCC3)nc12] |
| 522 | 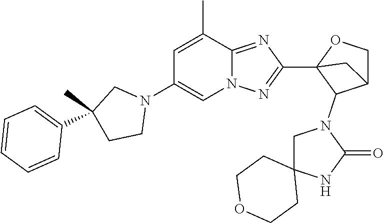 [SMILES: Cc1cc(N2CC[C@](C)(c3ccccc3)C2)cn2nc(C34CC(CO3)C4N3CC4(CCOCC4)NC3=O)nc12] |
| 523 | 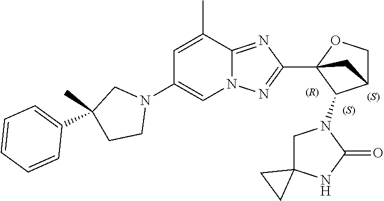 |
| 524 | 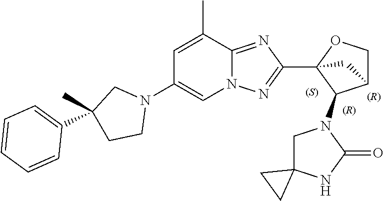 |
| 525 | 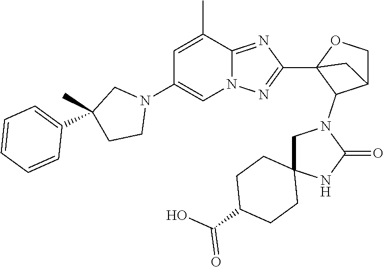 |
| 526 | 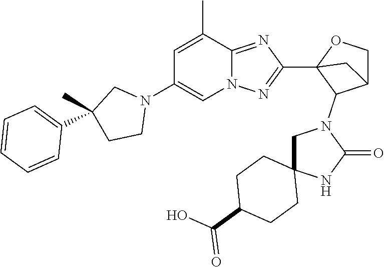 [SMILES: Cc1cc(N2CC[C@](C)(c3ccccc3)C2)cn2nc(C34CC(CO3)C4N3C[C@]4(CC[C@@H](C(=O)O)CC4)NC3=O)nc12] |
| 527 | 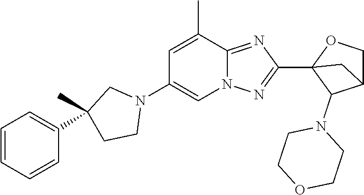 [SMILES: Cc1cc(N2CC[C@](C)(c3ccccc3)C2)cn2nc(C34CC(CO3)C4N3CCOCC3)nc12] |
| 528 | 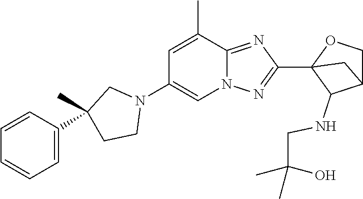 [SMILES: Cc1cc(N2CC[C@](C)(c3ccccc3)C2)cn2nc(C34CC(CO3)C4NCC(C)(C)O)nc12] |
| 529 | 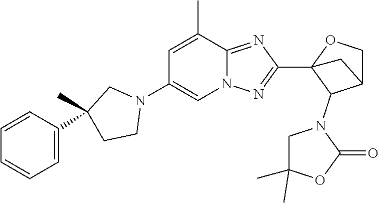 [SMILES: Cc1cc(N2CC[C@](C)(c3ccccc3)C2)cn2nc(C34CC(CO3)C4N3CC(C)(C)OC3=O)nc12] |
| 530 | 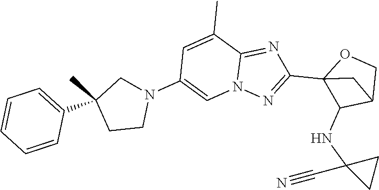 [SMILES: Cc1cc(N2CC[C@](C)(c3ccccc3)C2)cn2nc(C34CC(CO3)C4NC3(C#N)CC3)nc12] |
| 531 | 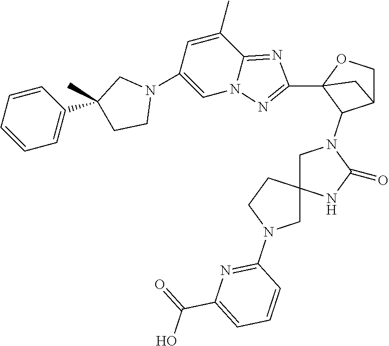 [SMILES: Cc1cc(N2CC[C@](C)(c3ccccc3)C2)cn2nc(C34CC(CO3)C4N3CC4(CCN(c5cccc(C(=O)O)n5)C4)NC3=O)nc12] |
| 532 | 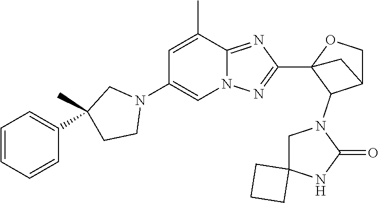 [SMILES: Cc1cc(N2CC[C@](C)(c3ccccc3)C2)cn2nc(C34CC(CO3)C4N3CC4(CCC4)NC3=O)nc12] |
| 533 | 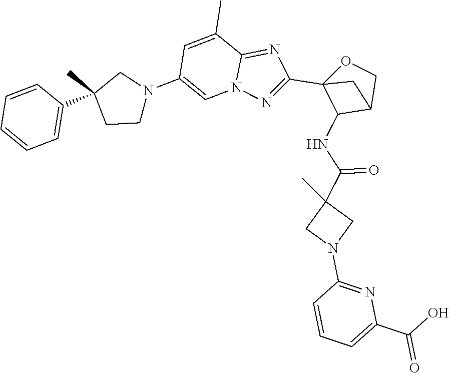 [SMILES: Cc1cc(N2CC[C@](C)(c3ccccc3)C2)cn2nc(C34CC(CO3)C4NC(=O)C3(C)CN(c4cccc(C(=O)O)n4)C3)nc12] |
| 536 | 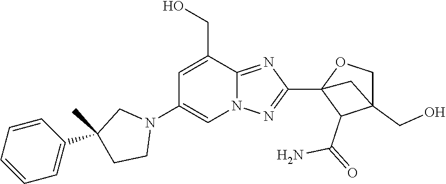 [SMILES: C[C@]1(c2ccccc2)CCN(c2cc(CO)c3nc(C45CC(CO)(CO4)C5C(N)=O)nn3c2)C1] |
| 537 | 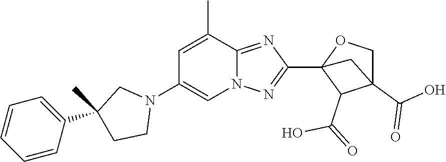 [SMILES: Cc1cc(N2CC[C@](C)(c3ccccc3)C2)cn2nc(C34CC(C(=O)O)(CO3)C4C(=O)O)nc12] |
| 538 | 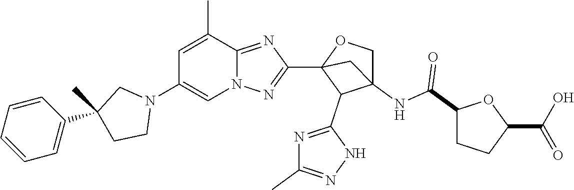 |
| 539 | 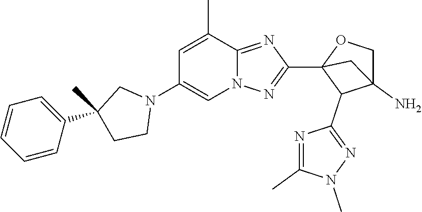 [SMILES: Cc1cc(N2CC[C@](C)(c3ccccc3)C2)cn2nc(C34CC(N)(CO3)C4c3nc(C)n(C)n3)nc12] |
| 540 | 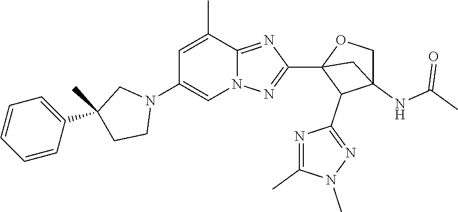 [SMILES: CC(=O)NC12COC(c3nc4c(C)cc(N5CC[C@](C)(c6ccccc6)C5)cn4n3)(C1)C2c1nc(C)n(C)n1] |
| 541 | 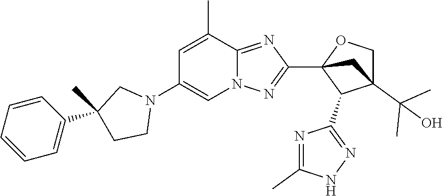 [SMILES: Cc1nc([C@H]2[C@@]3(C(C)(C)O)CO[C@]2(c2nc4c(C)cc(N5CC[C@](C)(c6ccccc6)C5)cn4n2)C3)n[nH]1] |
| 542 | 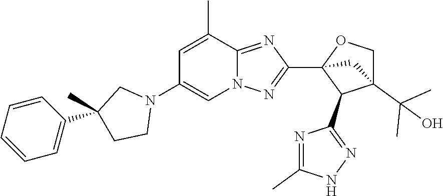 [SMILES: Cc1nc([C@@H]2[C@]3(C(C)(C)O)CO[C@@]2(c2nc4c(C)cc(N5CC[C@](C)(c6ccccc6)C5)cn4n2)C3)n[nH]1] |
| 543 | 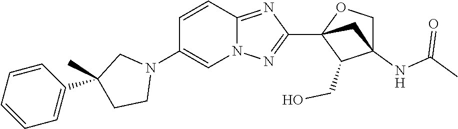 [SMILES: CC(=O)N[C@]12CO[C@](c3nc4ccc(N5CC[C@](C)(c6ccccc6)C5)cn4n3)(C1)[C@H]2CO] |
| 544 | 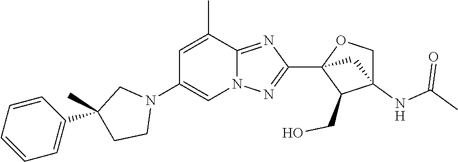 [SMILES: CC(=O)N[C@@]12CO[C@@](c3nc4c(C)cc(N5CC[C@](C)(c6ccccc6)C5)cn4n3)(C1)[C@@H]2CO] |
| 545 | 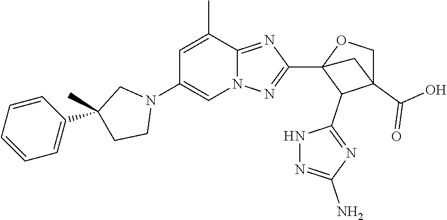 [SMILES: Cc1cc(N2CC[C@](C)(c3ccccc3)C2)cn2nc(C34CC(C(=O)O)(CO3)C4c3nc(N)n[nH]3)nc12] |
| 546 | 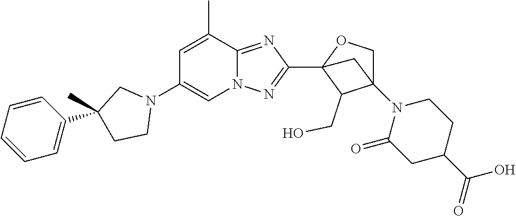 [SMILES: Cc1cc(N2CC[C@](C)(c3ccccc3)C2)cn2nc(C34CC(N5CCC(C(=O)O)CC5=O)(CO3)C4CO)nc12] |
| 547 | 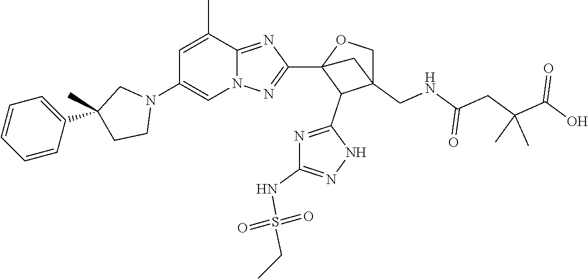 [SMILES: CCS(=O)(=O)Nc1n[nH]c(C2C3(CNC(=O)CC(C)(C)C(=O)O)COC2(c2nc4c(C)cc(N5CC[C@](C)(c6ccccc6)C5)cn4n2)C3)n1] |
| 548 | 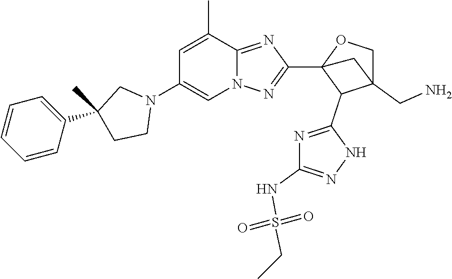 [SMILES: CCS(=O)(=O)Nc1n[nH]c(C2C3(CN)COC2(c2nc4c(C)cc(N5CC[C@](C)(c6ccccc6)C5)cn4n2)C3)n1] |
| 549 | 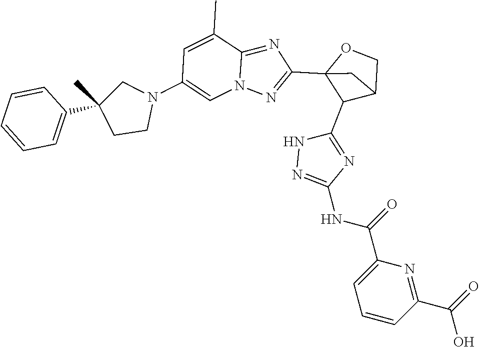 [SMILES: Cc1cc(N2CC[C@](C)(c3ccccc3)C2)cn2nc(C34CC(CO3)C4c3nc(NC(=O)c4cccc(C(=O)O)n4)n[nH]3)nc12] |
| 550 | 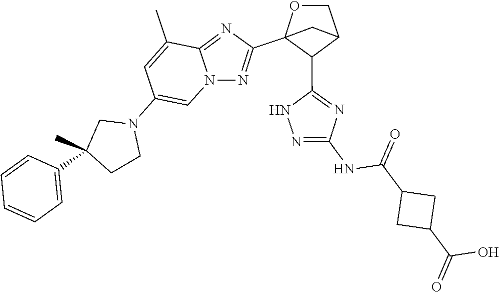 [SMILES: Cc1cc(N2CC[C@](C)(c3ccccc3)C2)cn2nc(C34CC(CO3)C4c3nc(NC(=O)C4CC(C(=O)O)C4)n[nH]3)nc12] |
| 551 | 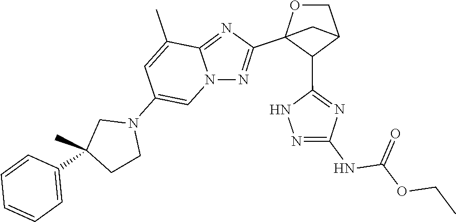 [SMILES: CCOC(=O)Nc1n[nH]c(C2C3COC2(c2nc4c(C)cc(N5CC[C@](C)(c6ccccc6)C5)cn4n2)C3)n1] |
| 552 | 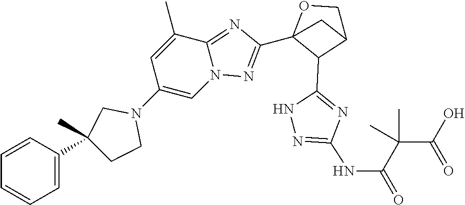 [SMILES: Cc1cc(N2CC[C@](C)(c3ccccc3)C2)cn2nc(C34CC(CO3)C4c3nc(NC(=O)C(C)(C)C(=O)O)n[nH]3)nc12] |
| 553 | 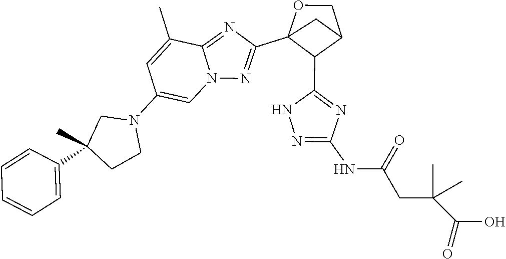 [SMILES: Cc1cc(N2CC[C@](C)(c3ccccc3)C2)cn2nc(C34CC(CO3)C4c3nc(NC(=O)CC(C)(C)C(=O)O)n[nH]3)nc12] |
| 554 | 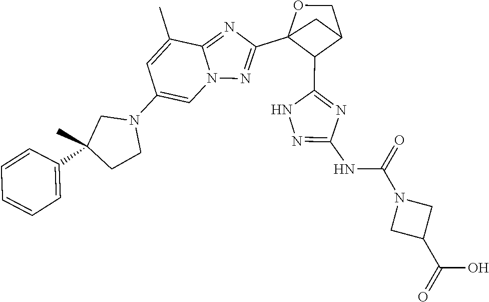 [SMILES: Cc1cc(N2CC[C@](C)(c3ccccc3)C2)cn2nc(C34CC(CO3)C4c3nc(NC(=O)N4CC(C(=O)O)C4)n[nH]3)nc12] |
| 555 | 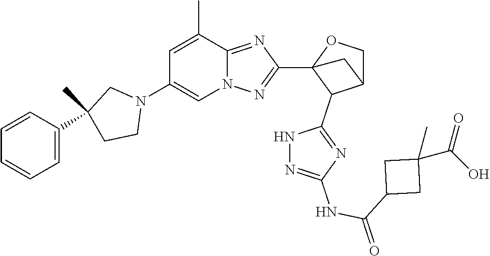 [SMILES: Cc1cc(N2CC[C@](C)(c3ccccc3)C2)cn2nc(C34CC(CO3)C4c3nc(NC(=O)C4CC(C)(C(=O)O)C4)n[nH]3)nc12] |
| 556 | 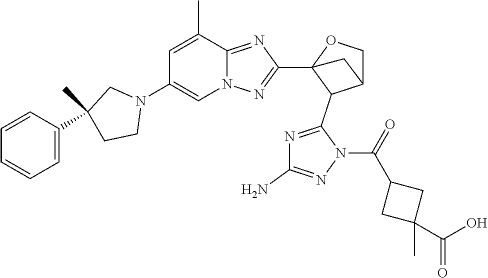 [SMILES: Cc1cc(N2CC[C@](C)(c3ccccc3)C2)cn2nc(C34CC(CO3)C4c3nc(N)nn3C(=O)C3CC(C)(C(=O)O)C3)nc12] |
| 557 | 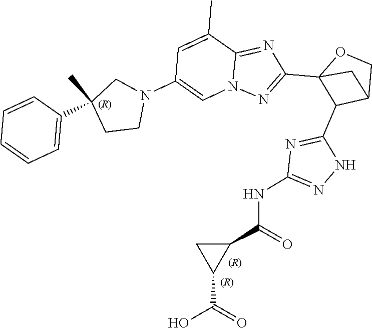 |
| 558 | 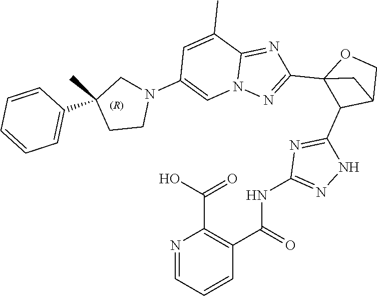 |
| 560 | 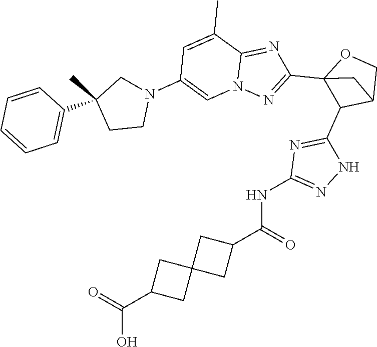 [SMILES: Cc1cc(N2CC[C@](C)(c3ccccc3)C2)cn2nc(C34CC(CO3)C4c3nc(NC(=O)C4CC5(CC(C(=O)O)C5)C4)n[nH]3)nc12] |
| 561 | 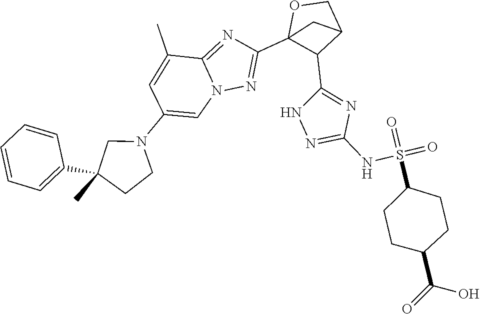 |
| 562 | 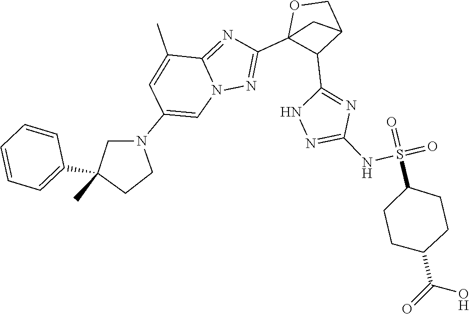 |
| 563 | 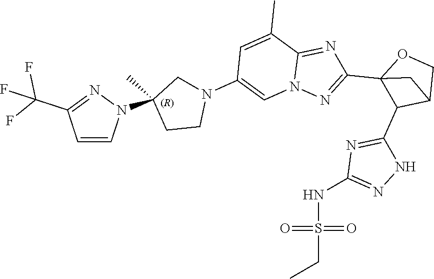 |
| 564 | 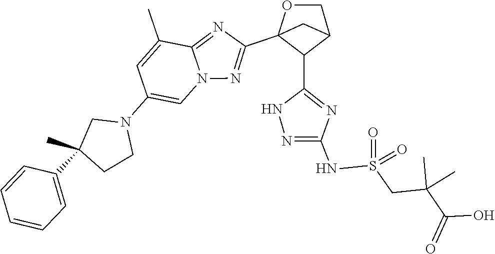 [SMILES: Cc1cc(N2CC[C@](C)(c3ccccc3)C2)cn2nc(C34CC(CO3)C4c3nc(NS(=O)(=O)CC(C)(C)C(=O)O)n[nH]3)nc12] |
| 565 | 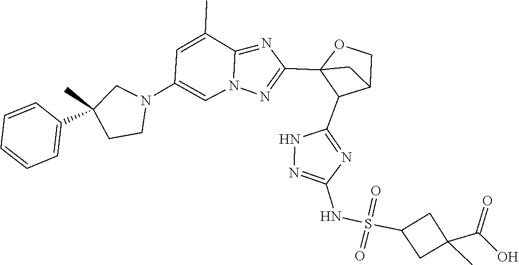 [SMILES: Cc1cc(N2CC[C@](C)(c3ccccc3)C2)cn2nc(C34CC(CO3)C4c3nc(NS(=O)(=O)C4CC(C)(C(=O)O)C4)n[nH]3)nc12] |
| 566 | 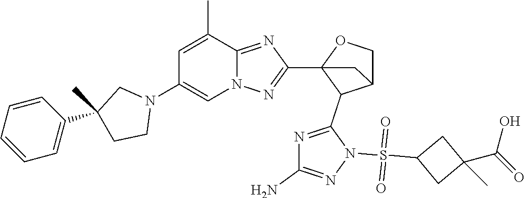 [SMILES: Cc1cc(N2CC[C@](C)(c3ccccc3)C2)cn2nc(C34CC(CO3)C4c3nc(N)nn3S(=O)(=O)C3CC(C)(C(=O)O)C3)nc12] |
| 567 | 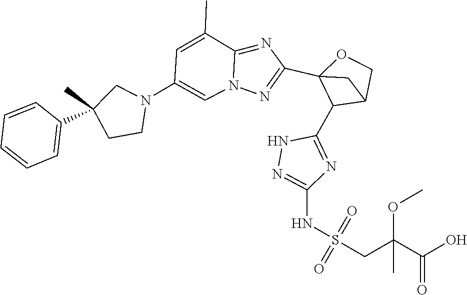 [SMILES: COC(C)(CS(=O)(=O)Nc1n[nH]c(C2C3COC2(c2nc4c(C)cc(N5CC[C@](C)(c6ccccc6)C5)cn4n2)C3)n1)C(=O)O] |
| 568 | 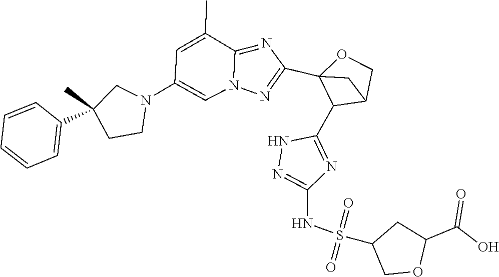 [SMILES: Cc1cc(N2CC[C@](C)(c3ccccc3)C2)cn2nc(C34CC(CO3)C4c3nc(NS(=O)(=O)C4COC(C(=O)O)C4)n[nH]3)nc12] |
| 569 | 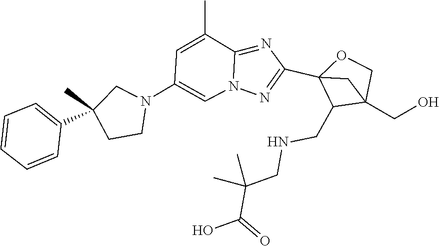 [SMILES: Cc1cc(N2CC[C@](C)(c3ccccc3)C2)cn2nc(C34CC(CO)(CO3)C4CNCC(C)(C)C(=O)O)nc12] |
| 570 | 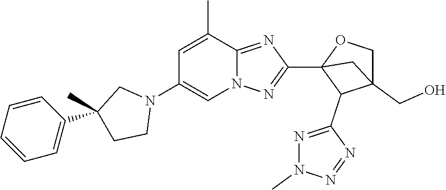 [SMILES: Cc1cc(N2CC[C@](C)(c3ccccc3)C2)cn2nc(C34CC(CO)(CO3)C4c3nnn(C)n3)nc12] |
| 571 | 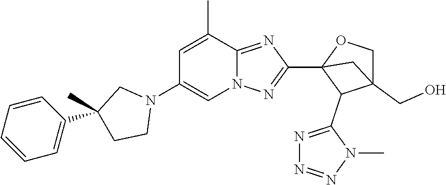 [SMILES: Cc1cc(N2CC[C@](C)(c3ccccc3)C2)cn2nc(C34CC(CO)(CO3)C4c3nnnn3C)nc12] |
| 572 | 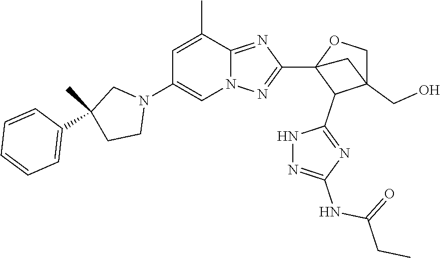 [SMILES: CCC(=O)Nc1n[nH]c(C2C3(CO)COC2(c2nc4c(C)cc(N5CC[C@](C)(c6ccccc6)C5)cn4n2)C3)n1] |
| 573 | 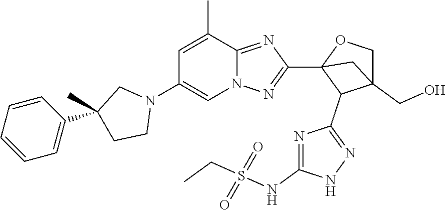 [SMILES: CCS(=O)(=O)Nc1nc(C2C3(CO)COC2(c2nc4c(C)cc(N5CC[C@](C)(c6ccccc6)C5)cn4n2)C3)n[nH]1] |
| 574 | 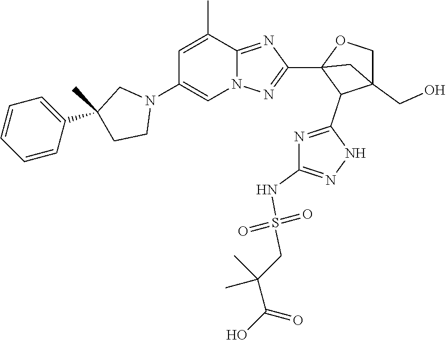 [SMILES: Cc1cc(N2CC[C@](C)(c3ccccc3)C2)cn2nc(C34CC(CO)(CO3)C4c3nc(NS(=O)(=O)CC(C)(C)C(=O)O)n[nH]3)nc12] |
| 575 | 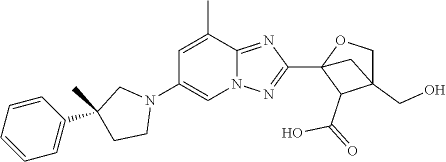 [SMILES: Cc1cc(N2CC[C@](C)(c3ccccc3)C2)cn2nc(C34CC(CO)(CO3)C4C(=O)O)nc12] |
| 576 | 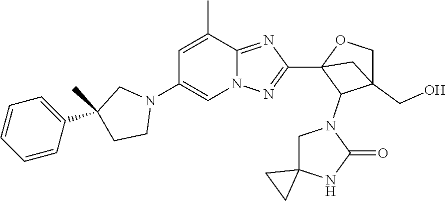 [SMILES: Cc1cc(N2CC[C@](C)(c3ccccc3)C2)cn2nc(C34CC(CO)(CO3)C4N3CC4(CC4)NC3=O)nc12] |
| 577 | 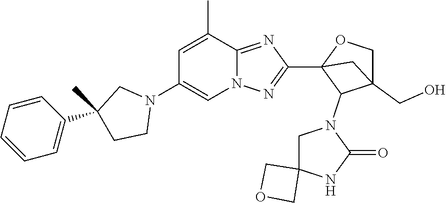 [SMILES: Cc1cc(N2CC[C@](C)(c3ccccc3)C2)cn2nc(C34CC(CO)(CO3)C4N3CC4(COC4)NC3=O)nc12] |
| 579 | 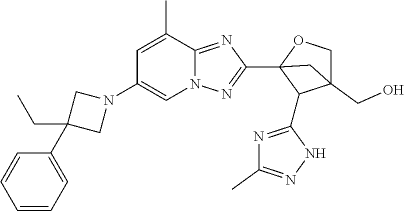 [SMILES: CCC1(c2ccccc2)CN(c2cc(C)c3nc(C45CC(CO)(CO4)C5c4nc(C)n[nH]4)nn3c2)C1] |
| 580 | 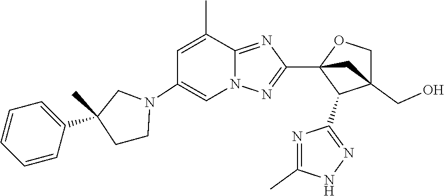 [SMILES: Cc1nc([C@H]2[C@@]3(CO)CO[C@]2(c2nc4c(C)cc(N5CC[C@](C)(c6ccccc6)C5)cn4n2)C3)n[nH]1] |
| 581 | 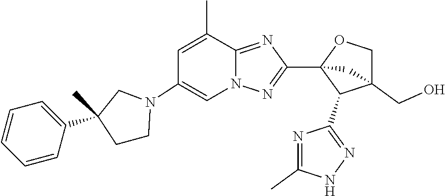 [SMILES: Cc1nc([C@H]2[C@]3(CO)CO[C@@]2(c2nc4c(C)cc(N5CC[C@](C)(c6ccccc6)C5)cn4n2)C3)n[nH]1] |
| 582 | 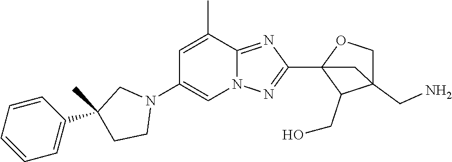 [SMILES: Cc1cc(N2CC[C@](C)(c3ccccc3)C2)cn2nc(C34CC(CN)(CO3)C4CO)nc12] |
| 587 | 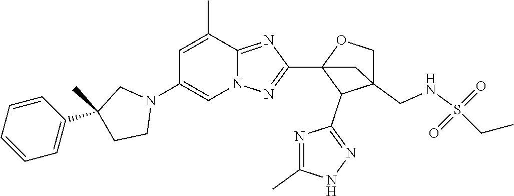 [SMILES: CCS(=O)(=O)NCC12COC(c3nc4c(C)cc(N5CC[C@](C)(c6ccccc6)C5)cn4n3)(C1)C2c1n[nH]c(C)n1] |
| 588 | 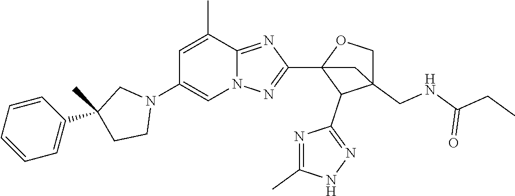 [SMILES: CCC(=O)NCC12COC(c3nc4c(C)cc(N5CC[C@](C)(c6ccccc6)C5)cn4n3)(C1)C2c1n[nH]c(C)n1] |
| 589 | 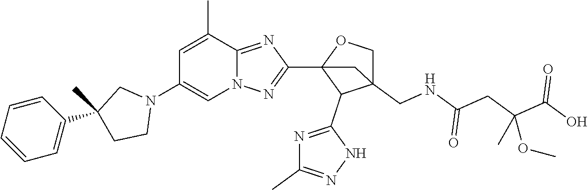 [SMILES: COC(C)(CC(=O)NCC12COC(c3nc4c(C)cc(N5CC[C@](C)(c6ccccc6)C5)cn4n3)(C1)C2c1nc(C)n[nH]1)C(=O)O] |
| 590 | 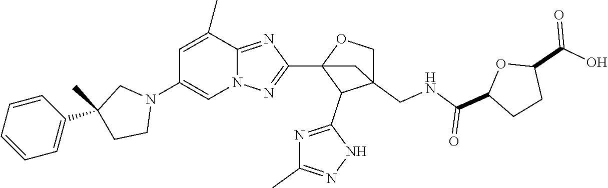 |

TABLE 2

| Cmpd | |
|---|---|
| # | Structure |
| 14 | 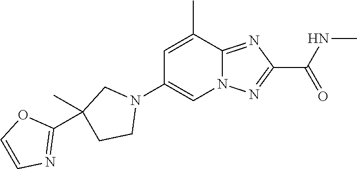 [SMILES: CNC(=O)c1nc2c(C)cc(N3CCC(C)(c4ncco4)C3)cn2n1] |
| 15 | 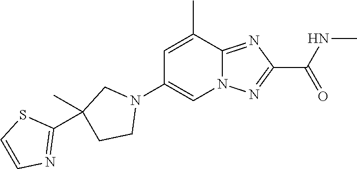 [SMILES: CNC(=O)c1nc2c(C)cc(N3CCC(C)(c4nccs4)C3)cn2n1] |
| 18 | 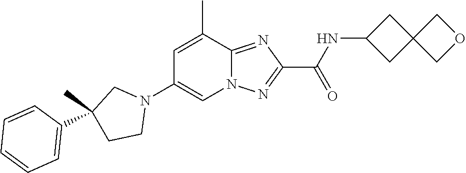 [SMILES: Cc1cc(N2CC[C@](C)(c3ccccc3)C2)cn2nc(C(=O)NC3CC4(COC4)C3)nc12] |
| 100 | 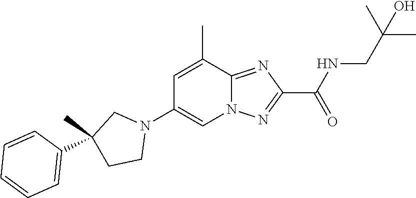 [SMILES: Cc1cc(N2CC[C@](C)(c3ccccc3)C2)cn2nc(C(=O)NCC(C)(C)O)nc12] |
| 101 | 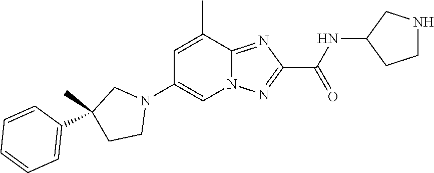 [SMILES: Cc1cc(N2CC[C@](C)(c3ccccc3)C2)cn2nc(C(=O)NC3CCNC3)nc12] |
| 102 | 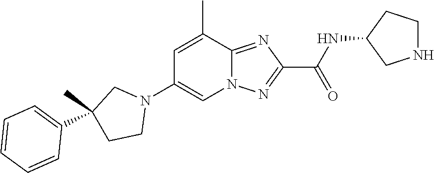 [SMILES: Cc1cc(N2CC[C@](C)(c3ccccc3)C2)cn2nc(C(=O)N[C@@H]3CCNC3)nc12] |
| 103 | 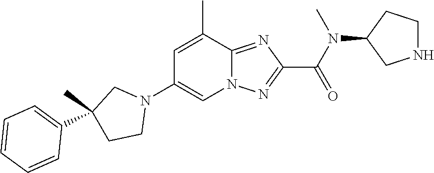 [SMILES: Cc1cc(N2CC[C@](C)(c3ccccc3)C2)cn2nc(C(=O)N(C)[C@H]3CCNC3)nc12] |
| 104 | 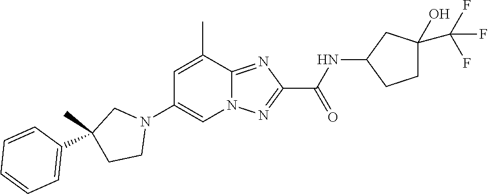 [SMILES: Cc1cc(N2CC[C@](C)(c3ccccc3)C2)cn2nc(C(=O)NC3CCC(O)(C(F)(F)F)C3)nc12] |
| 105 | 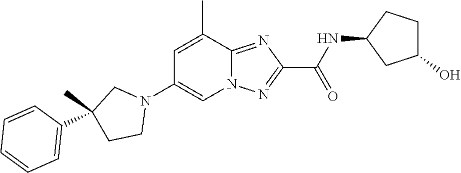 [SMILES: Cc1cc(N2CC[C@](C)(c3ccccc3)C2)cn2nc(C(=O)N[C@H]3CC[C@H](O)C3)nc12] |
| 106 | 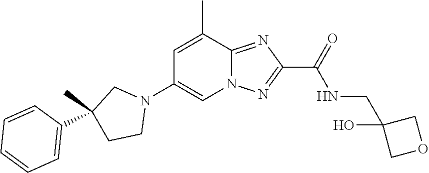 [SMILES: Cc1cc(N2CC[C@](C)(c3ccccc3)C2)cn2nc(C(=O)NCC3(O)COC3)nc12] |
| 107 | 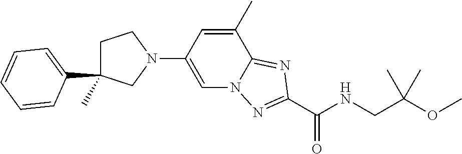 [SMILES: COC(C)(C)CNC(=O)c1nc2c(C)cc(N3CC[C@](C)(c4ccccc4)C3)cn2n1] |
| 108 | 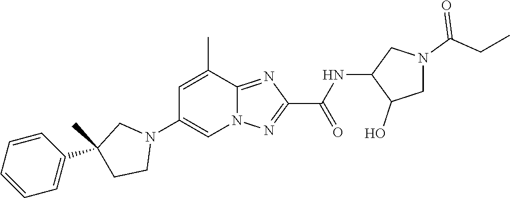 [SMILES: CCC(=O)N1CC(O)C(NC(=O)c2nc3c(C)cc(N4CC[C@](C)(c5ccccc5)C4)cn3n2)C1] |
| 109 | 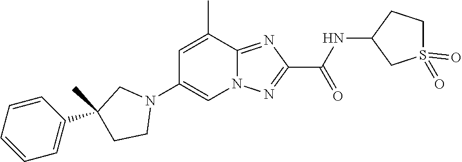 [SMILES: Cc1cc(N2CC[C@](C)(c3ccccc3)C2)cn2nc(C(=O)NC3CCS(=O)(=O)C3)nc12] |
| 110 | 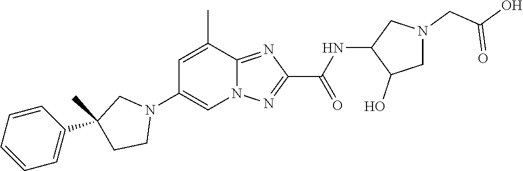 [SMILES: Cc1cc(N2CC[C@](C)(c3ccccc3)C2)cn2nc(C(=O)NC3CN(CC(=O)O)CC3O)nc12] |
| 111 | 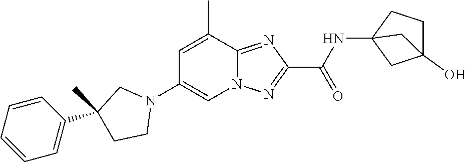 [SMILES: Cc1cc(N2CC[C@](C)(c3ccccc3)C2)cn2nc(C(=O)NC34CCC(O)(C3)C4)nc12] |
| 112 | 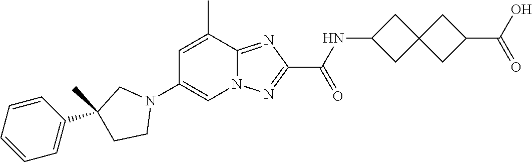 [SMILES: Cc1cc(N2CC[C@](C)(c3ccccc3)C2)cn2nc(C(=O)NC3CC4(C3)CC(C(=O)O)C4)nc12] |
| 114 | 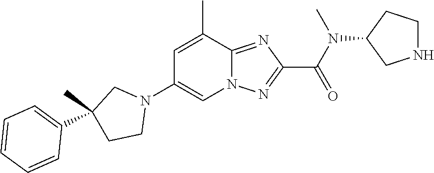 [SMILES: Cc1cc(N2CC[C@](C)(c3ccccc3)C2)cn2nc(C(=O)N(C)[C@@H]3CCNC3)nc12] |
| 116 | 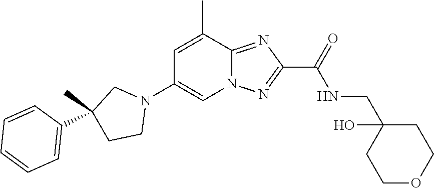 [SMILES: Cc1cc(N2CC[C@](C)(c3ccccc3)C2)cn2nc(C(=O)NCC3(O)CCOCC3)nc12] |
| 117 | 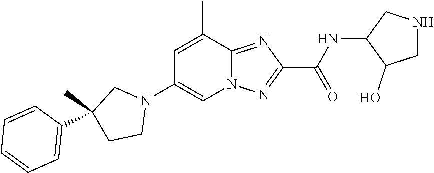 [SMILES: Cc1cc(N2CC[C@](C)(c3ccccc3)C2)cn2nc(C(=O)NC3CNCC3O)nc12] |
| 118 | 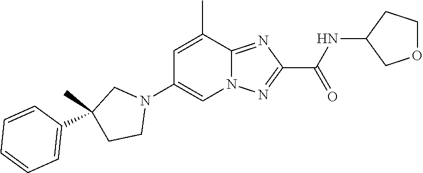 [SMILES: Cc1cc(N2CC[C@](C)(c3ccccc3)C2)cn2nc(C(=O)NC3CCOC3)nc12] |
| 119 | 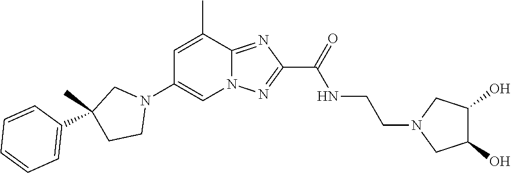 [SMILES: Cc1cc(N2CC[C@](C)(c3ccccc3)C2)cn2nc(C(=O)NCCN3C[C@H](O)[C@@H](O)C3)nc12] |
| 120 | 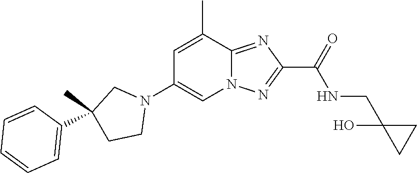 [SMILES: Cc1cc(N2CC[C@](C)(c3ccccc3)C2)cn2nc(C(=O)NCC3(O)CC3)nc12] |
| 121 | 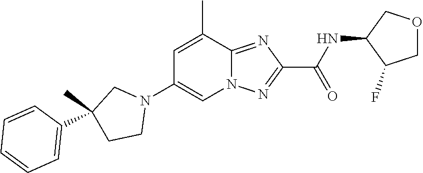 [SMILES: Cc1cc(N2CC[C@](C)(c3ccccc3)C2)cn2nc(C(=O)N[C@H]3COC[C@@H]3F)nc12] |
| 127 | 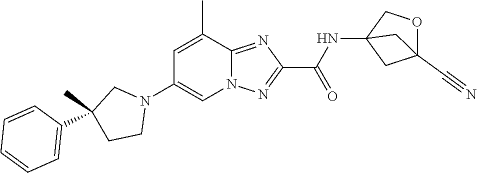 [SMILES: Cc1cc(N2CC[C@](C)(c3ccccc3)C2)cn2nc(C(=O)NC34COC(C#N)(C3)C4)nc12] |
| 129 | 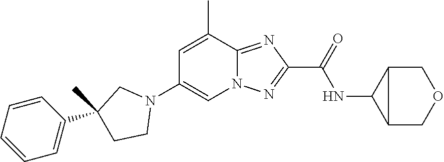 [SMILES: Cc1cc(N2CC[C@](C)(c3ccccc3)C2)cn2nc(C(=O)NC3C4COCC43)nc12] |
| 130 | 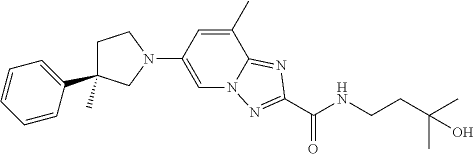 [SMILES: Cc1cc(N2CC[C@](C)(c3ccccc3)C2)cn2nc(C(=O)NCCC(C)(C)O)nc12] |
| 131 | 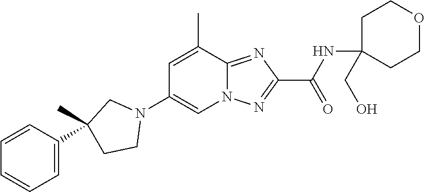 [SMILES: Cc1cc(N2CC[C@](C)(c3ccccc3)C2)cn2nc(C(=O)NC3(CO)CCOCC3)nc12] |
| 132 | 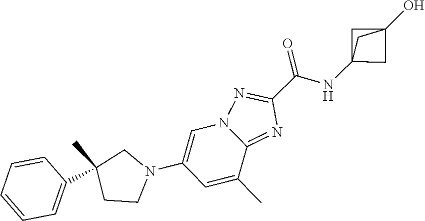 [SMILES: Cc1cc(N2CC[C@](C)(c3ccccc3)C2)cn2nc(C(=O)NC34CC(O)(C3)C4)nc12] |
| 133 | 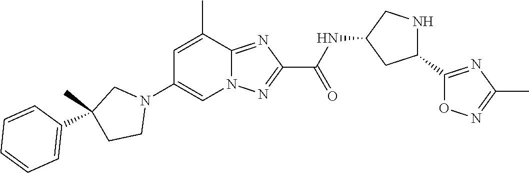 [SMILES: Cc1noc([C@@H]2C[C@H](NC(=O)c3nc4c(C)cc(N5CC[C@](C)(c6ccccc6)C5)cn4n3)CN2)n1] |
| 134 | 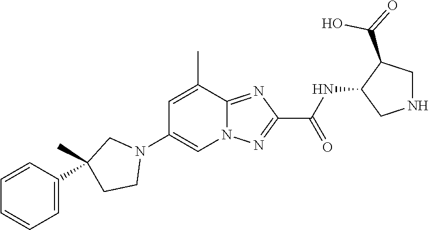 [SMILES: Cc1cc(N2CC[C@](C)(c3ccccc3)C2)cn2nc(C(=O)N[C@H]3CNC[C@@H]3C(=O)O)nc12] |
| 136 | 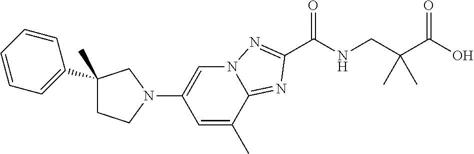 [SMILES: Cc1cc(N2CC[C@](C)(c3ccccc3)C2)cn2nc(C(=O)NCC(C)(C)C(=O)O)nc12] |
| 137 | 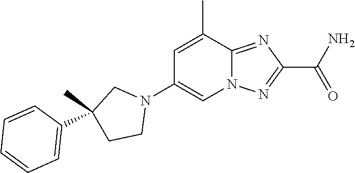 [SMILES: Cc1cc(N2CC[C@](C)(c3ccccc3)C2)cn2nc(C(N)=O)nc12] |
| 138 | 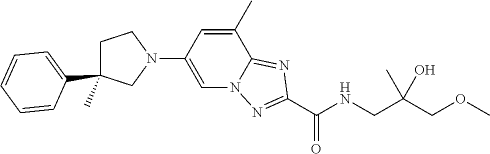 [SMILES: COCC(C)(O)CNC(=O)c1nc2c(C)cc(N3CC[C@](C)(c4ccccc4)C3)cn2n1] |
| 139 | 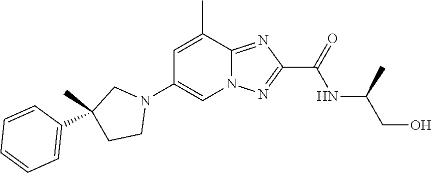 [SMILES: Cc1cc(N2CC[C@](C)(c3ccccc3)C2)cn2nc(C(=O)N[C@@H](C)CO)nc12] |
| 140 | 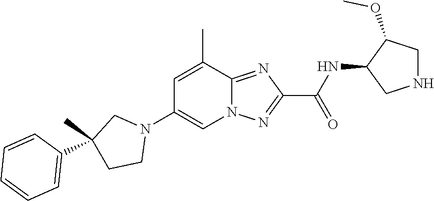 [SMILES: CO[C@@H]1CNC[C@H]1NC(=O)c1nc2c(C)cc(N3CC[C@](C)(c4ccccc4)C3)cn2n1] |
| 141 | 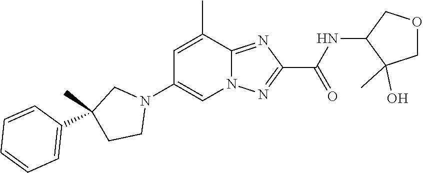 [SMILES: Cc1cc(N2CC[C@](C)(c3ccccc3)C2)cn2nc(C(=O)NC3COCC3(C)O)nc12] |
| 142 | 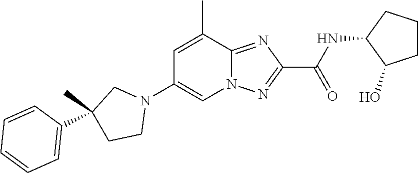 [SMILES: Cc1cc(N2CC[C@](C)(c3ccccc3)C2)cn2nc(C(=O)N[C@@H]3CCC[C@@H]3O)nc12] |
| 145 | 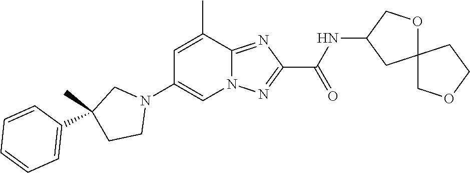 [SMILES: Cc1cc(N2CC[C@](C)(c3ccccc3)C2)cn2nc(C(=O)NC3COC4(CCOC4)C3)nc12] |
| 148 | 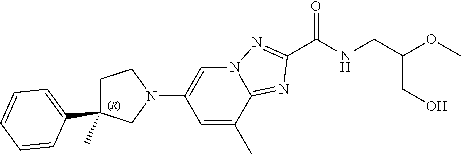 |
| 149 | 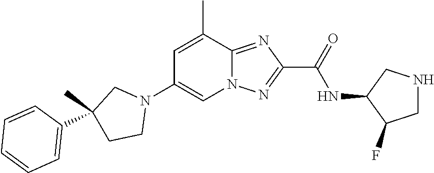 [SMILES: Cc1cc(N2CC[C@](C)(c3ccccc3)C2)cn2nc(C(=O)N[C@H]3CNC[C@H]3F)nc12] |
| 150 | 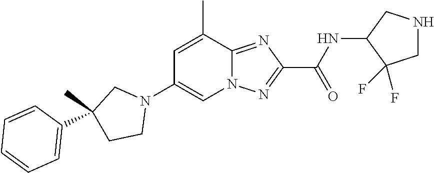 [SMILES: Cc1cc(N2CC[C@](C)(c3ccccc3)C2)cn2nc(C(=O)NC3CNCC3(F)F)nc12] |
| 151 | 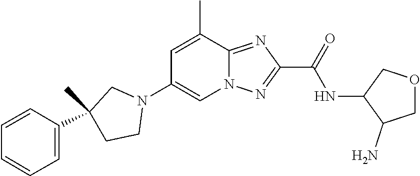 [SMILES: Cc1cc(N2CC[C@](C)(c3ccccc3)C2)cn2nc(C(=O)NC3COCC3N)nc12] |
| 152 | 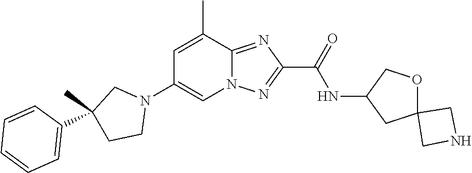 [SMILES: Cc1cc(N2CC[C@](C)(c3ccccc3)C2)cn2nc(C(=O)NC3COC4(CNC4)C3)nc12] |
| 153 | 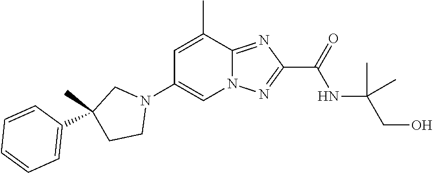 [SMILES: Cc1cc(N2CC[C@](C)(c3ccccc3)C2)cn2nc(C(=O)NC(C)(C)CO)nc12] |
| 154 | 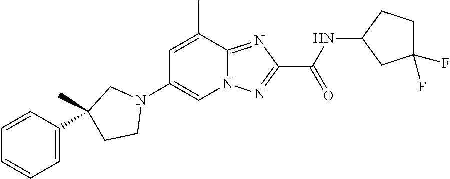 [SMILES: Cc1cc(N2CC[C@](C)(c3ccccc3)C2)cn2nc(C(=O)NC3CCC(F)(F)C3)nc12] |
| 155 | 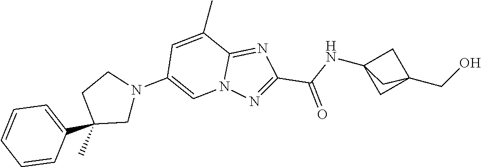 [SMILES: Cc1cc(N2CC[C@](C)(c3ccccc3)C2)cn2nc(C(=O)NC34CC(CO)(C3)C4)nc12] |
| 156 | 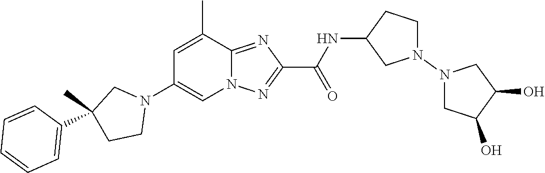 [SMILES: Cc1cc(N2CC[C@](C)(c3ccccc3)C2)cn2nc(C(=O)NC3CCN(N4C[C@@H](O)[C@@H](O)C4)C3)nc12] |
| 157 | 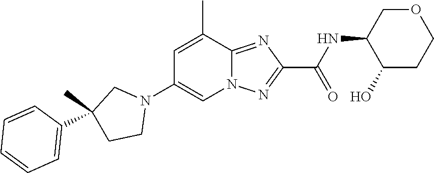 [SMILES: Cc1cc(N2CC[C@](C)(c3ccccc3)C2)cn2nc(C(=O)N[C@H]3COCC[C@@H]3O)nc12] |
| 160 | 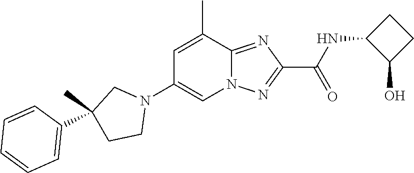 [SMILES: Cc1cc(N2CC[C@](C)(c3ccccc3)C2)cn2nc(C(=O)N[C@@H]3CC[C@H]3O)nc12] |
| 161 | 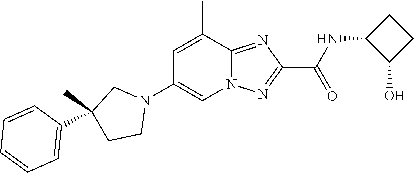 [SMILES: Cc1cc(N2CC[C@](C)(c3ccccc3)C2)cn2nc(C(=O)N[C@@H]3CC[C@@H]3O)nc12] |
| 162 | 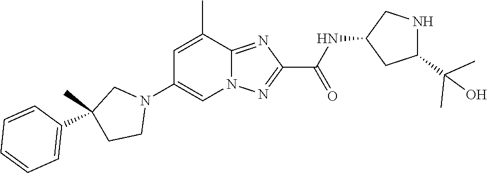 [SMILES: Cc1cc(N2CC[C@](C)(c3ccccc3)C2)cn2nc(C(=O)N[C@@H]3CN[C@H](C(C)(C)O)C3)nc12] |
| 163 | 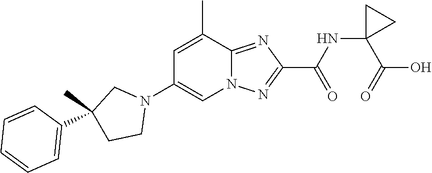 [SMILES: Cc1cc(N2CC[C@](C)(c3ccccc3)C2)cn2nc(C(=O)NC3(C(=O)O)CC3)nc12] |
| 164 | 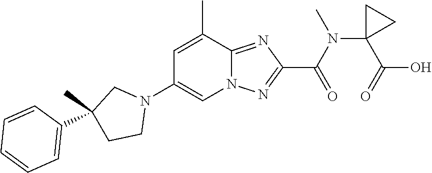 [SMILES: Cc1cc(N2CC[C@](C)(c3ccccc3)C2)cn2nc(C(=O)N(C)C3(C(=O)O)CC3)nc12] |
| 165 | 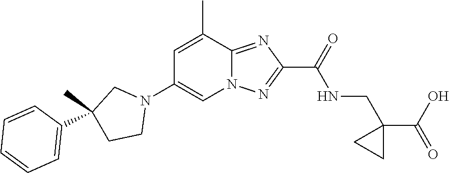 [SMILES: Cc1cc(N2CC[C@](C)(c3ccccc3)C2)cn2nc(C(=O)NCC3(C(=O)O)CC3)nc12] |
| 166 | 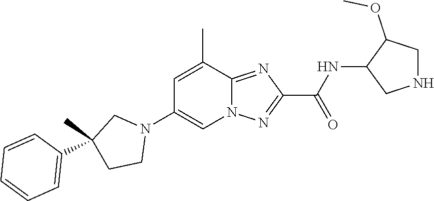 [SMILES: COC1CNCC1NC(=O)c1nc2c(C)cc(N3CC[C@](C)(c4ccccc4)C3)cn2n1] |
| 167 | 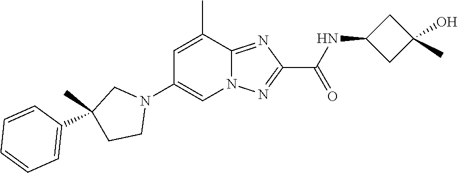 [SMILES: Cc1cc(N2CC[C@](C)(c3ccccc3)C2)cn2nc(C(=O)N[C@H]3C[C@](C)(O)C3)nc12] |
| 169 | 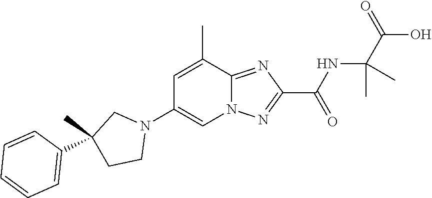 [SMILES: Cc1cc(N2CC[C@](C)(c3ccccc3)C2)cn2nc(C(=O)NC(C)(C)C(=O)O)nc12] |
| 170 | 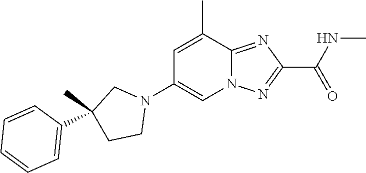 [SMILES: CNC(=O)c1nc2c(C)cc(N3CC[C@](C)(c4ccccc4)C3)cn2n1] |
| 171 | 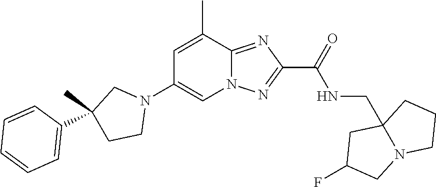 [SMILES: Cc1cc(N2CC[C@](C)(c3ccccc3)C2)cn2nc(C(=O)NCC34CCCN3CC(F)C4)nc12] |
| 172 | 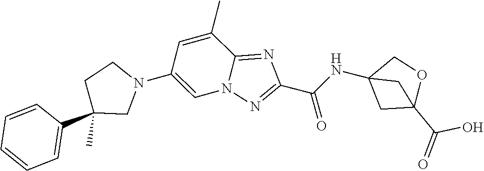 [SMILES: Cc1cc(N2CC[C@](C)(c3ccccc3)C2)cn2nc(C(=O)NC34COC(C(=O)O)(C3)C4)nc12] |
| 173 | 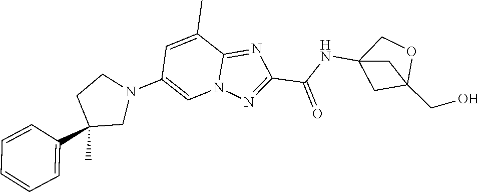 [SMILES: Cc1cc(N2CC[C@](C)(c3ccccc3)C2)cn2nc(C(=O)NC34COC(CO)(C3)C4)nc12] |
| 175 | 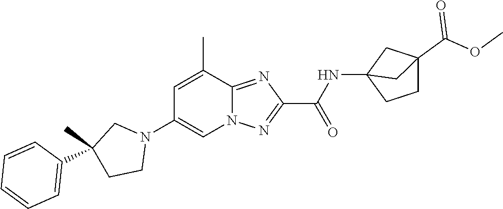 [SMILES: COC(=O)C12CCC(NC(=O)c3nc4c(C)cc(N5CC[C@](C)(c6ccccc6)C5)cn4n3)(C1)C2] |
| 179 | 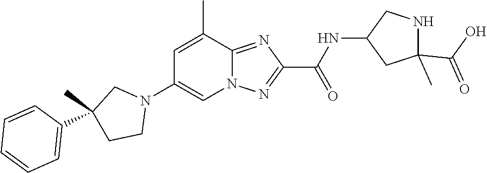 [SMILES: Cc1cc(N2CC[C@](C)(c3ccccc3)C2)cn2nc(C(=O)NC3CNC(C)(C(=O)O)C3)nc12] |
| 180 | 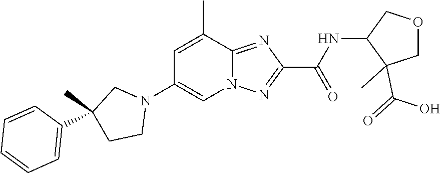 [SMILES: Cc1cc(N2CC[C@](C)(c3ccccc3)C2)cn2nc(C(=O)NC3COCC3(C)C(=O)O)nc12] |
| 181 | 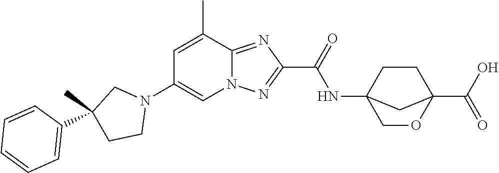 [SMILES: Cc1cc(N2CC[C@](C)(c3ccccc3)C2)cn2nc(C(=O)NC34CCC(C(=O)O)(C3)OC4)nc12] |
| 184 | 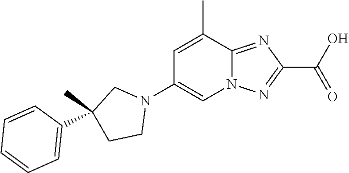 [SMILES: Cc1cc(N2CC[C@](C)(c3ccccc3)C2)cn2nc(C(=O)O)nc12] |
| 185 | 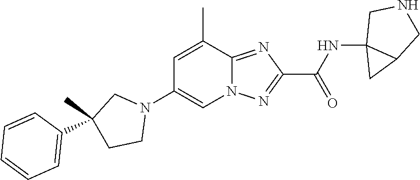 [SMILES: Cc1cc(N2CC[C@](C)(c3ccccc3)C2)cn2nc(C(=O)NC34CNCC3C4)nc12] |
| 186 | 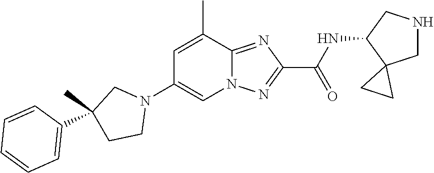 [SMILES: Cc1cc(N2CC[C@](C)(c3ccccc3)C2)cn2nc(C(=O)N[C@@H]3CNCC34CC4)nc12] |
| 187 | 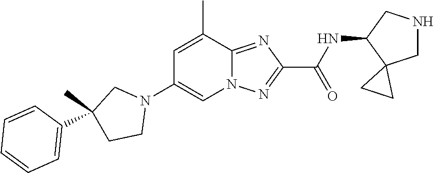 [SMILES: Cc1cc(N2CC[C@](C)(c3ccccc3)C2)cn2nc(C(=O)N[C@H]3CNCC34CC4)nc12] |
| 188 | 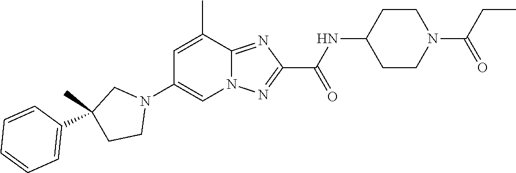 [SMILES: CCC(=O)N1CCC(NC(=O)c2nc3c(C)cc(N4CC[C@](C)(c5ccccc5)C4)cn3n2)CC1] |
| 189 | 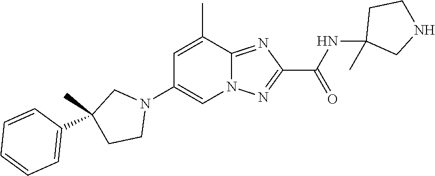 [SMILES: Cc1cc(N2CC[C@](C)(c3ccccc3)C2)cn2nc(C(=O)NC3(C)CCNC3)nc12] |
| 190 | 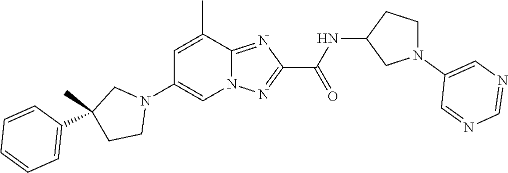 [SMILES: Cc1cc(N2CC[C@](C)(c3ccccc3)C2)cn2nc(C(=O)NC3CCN(c4cncnc4)C3)nc12] |
| 191 | 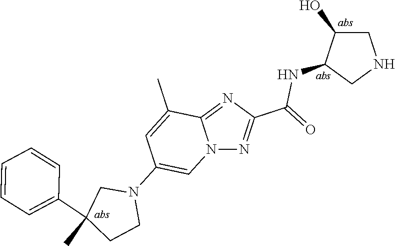 [SMILES: Cc1cc(N2CC[C@](C)(c3ccccc3)C2)cn2nc(C(=O)N[C@@H]3CNC[C@@H]3O)nc12] |
| 192 | 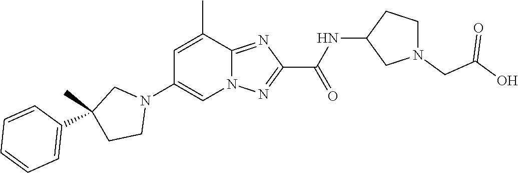 [SMILES: Cc1cc(N2CC[C@](C)(c3ccccc3)C2)cn2nc(C(=O)NC3CCN(CC(=O)O)C3)nc12] |
| 193 | 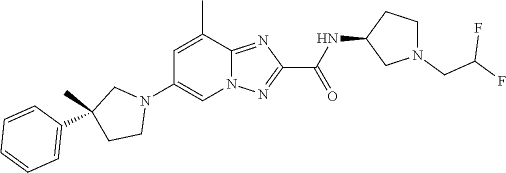 [SMILES: Cc1cc(N2CC[C@](C)(c3ccccc3)C2)cn2nc(C(=O)N[C@H]3CCN(CC(F)F)C3)nc12] |
| 198 | 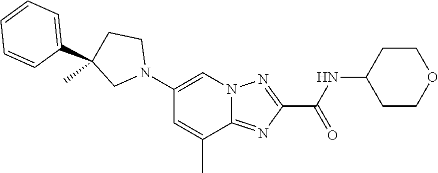 [SMILES: Cc1cc(N2CC[C@](C)(c3ccccc3)C2)cn2nc(C(=O)NC3CCOCC3)nc12] |
| 199 | 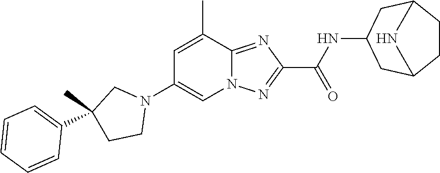 [SMILES: Cc1cc(N2CC[C@](C)(c3ccccc3)C2)cn2nc(C(=O)NC3CC4CCC(C3)N4)nc12] |
| 200 | 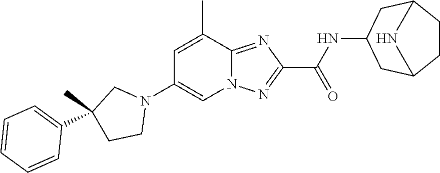 [SMILES: Cc1cc(N2CC[C@](C)(c3ccccc3)C2)cn2nc(C(=O)NC3CC4CCC(C3)N4)nc12] |
| 201 | 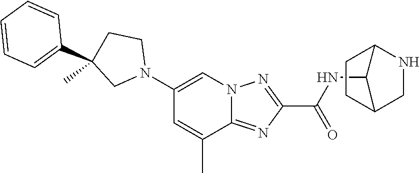 [SMILES: Cc1cc(N2CC[C@](C)(c3ccccc3)C2)cn2nc(C(=O)NC3C4CCC3NC4)nc12] |
| 202 | 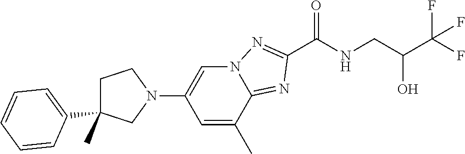 [SMILES: Cc1cc(N2CC[C@@](C)(c3ccccc3)C2)cn2nc(C(=O)NCC(O)C(F)(F)F)nc12] |
| 203 | 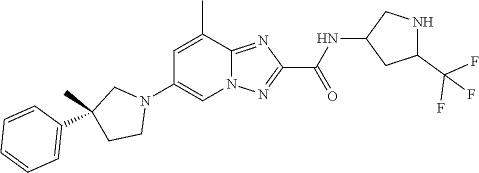 [SMILES: Cc1cc(N2CC[C@](C)(c3ccccc3)C2)cn2nc(C(=O)NC3CNC(C(F)(F)F)C3)nc12] |
| 204 | 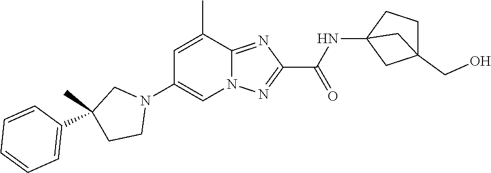 [SMILES: Cc1cc(N2CC[C@](C)(c3ccccc3)C2)cn2nc(C(=O)NC34CCC(CO)(C3)C4)nc12] |
| 205 | 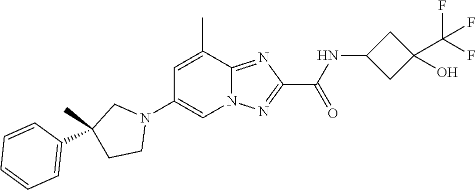 [SMILES: Cc1cc(N2CC[C@](C)(c3ccccc3)C2)cn2nc(C(=O)NC3CC(O)(C(F)(F)F)C3)nc12] |
| 206 | 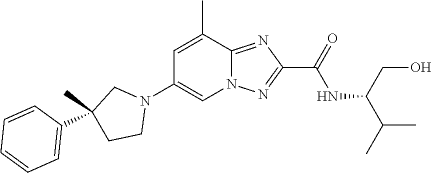 [SMILES: Cc1cc(N2CC[C@](C)(c3ccccc3)C2)cn2nc(C(=O)N[C@H](CO)C(C)C)nc12] |
| 207 | 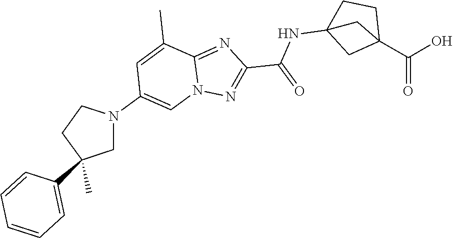 [SMILES: Cc1cc(N2CC[C@](C)(c3ccccc3)C2)cn2nc(C(=O)NC34CCC(C(=O)O)(C3)C4)nc12] |
| 208 | 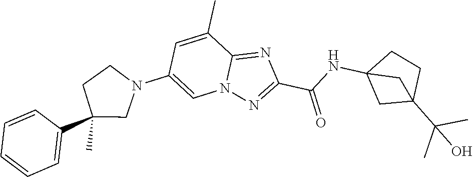 [SMILES: Cc1cc(N2CC[C@](C)(c3ccccc3)C2)cn2nc(C(=O)NC34CCC(C(C)(C)O)(C3)C4)nc12] |
| 209 | 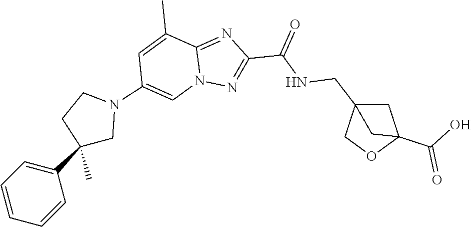 [SMILES: Cc1cc(N2CC[C@](C)(c3ccccc3)C2)cn2nc(C(=O)NCC34COC(C(=O)O)(C3)C4)nc12] |
| 212 | 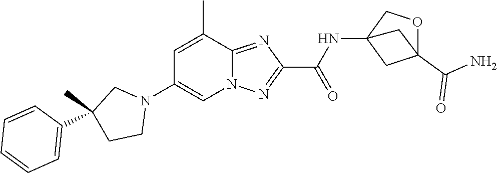 [SMILES: Cc1cc(N2CC[C@](C)(c3ccccc3)C2)cn2nc(C(=O)NC34COC(C(N)=O)(C3)C4)nc12] |
| 19 | 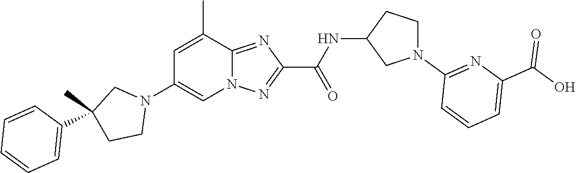 [SMILES: Cc1cc(N2CC[C@](C)(c3ccccc3)C2)cn2nc(C(=O)NC3CCN(c4cccc(C(=O)O)n4)C3)nc12] |
| 214 | 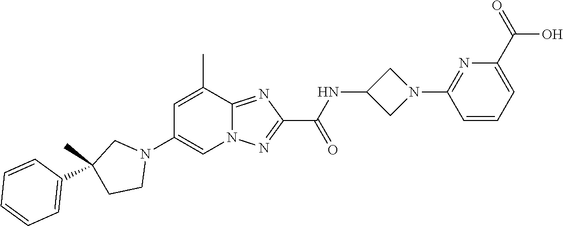 [SMILES: Cc1cc(N2CC[C@](C)(c3ccccc3)C2)cn2nc(C(=O)NC3CN(c4cccc(C(=O)O)n4)C3)nc12] |
| 217 | 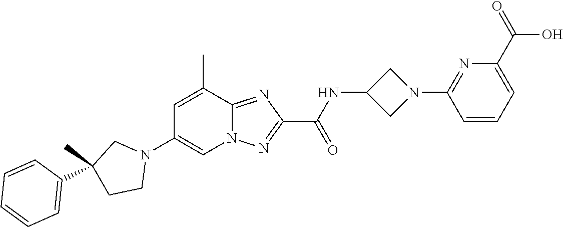 [SMILES: Cc1cc(N2CC[C@](C)(c3ccccc3)C2)cn2nc(C(=O)NC3CN(c4cccc(C(=O)O)n4)C3)nc12] |
| 219 | 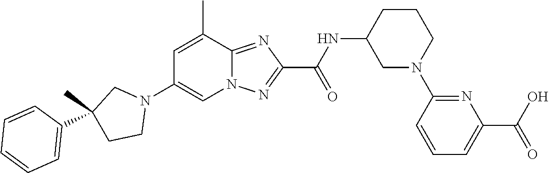 [SMILES: Cc1cc(N2CC[C@](C)(c3ccccc3)C2)cn2nc(C(=O)NC3CCCN(c4cccc(C(=O)O)n4)C3)nc12] |
| 220 | 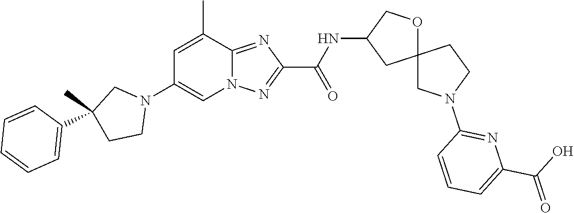 [SMILES: Cc1cc(N2CC[C@](C)(c3ccccc3)C2)cn2nc(C(=O)NC3COC4(CCN(c5cccc(C(=O)O)n5)C4)C3)nc12] |
| 223 | 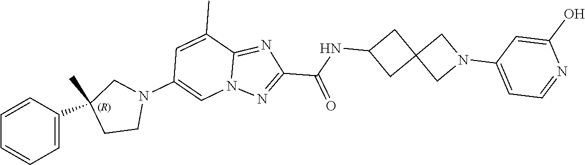 |
| 224 | 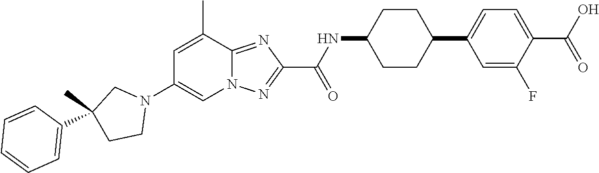 |
| 225 | 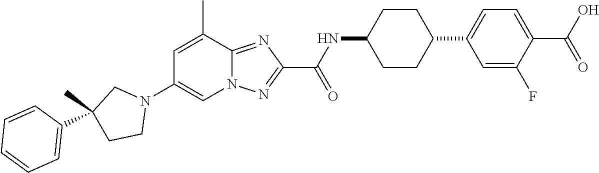 |
| 226 | 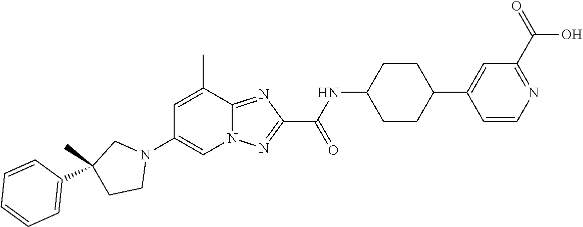 [SMILES: Cc1cc(N2CC[C@](C)(c3ccccc3)C2)cn2nc(C(=O)NC3CCC(c4ccnc(C(=O)O)c4)CC3)nc12] |
| 227 | 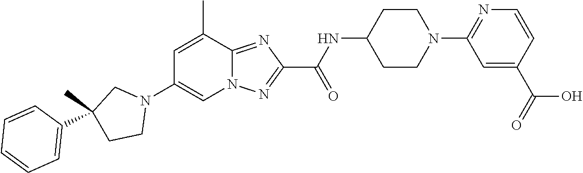 [SMILES: Cc1cc(N2CC[C@](C)(c3ccccc3)C2)cn2nc(C(=O)NC3CCN(c4cc(C(=O)O)ccn4)CC3)nc12] |
| 228 | 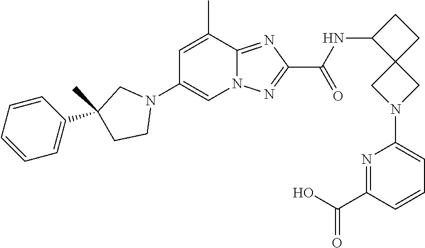 [SMILES: Cc1cc(N2CC[C@](C)(c3ccccc3)C2)cn2nc(C(=O)NC3CCC34CN(c3cccc(C(=O)O)n3)C4)nc12] |
| 229 | 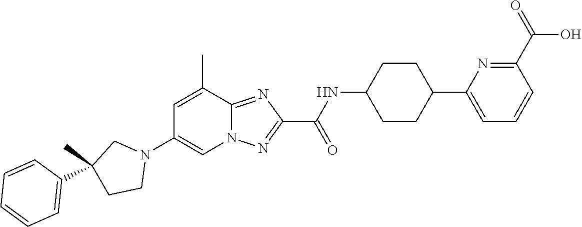 [SMILES: Cc1cc(N2CC[C@](C)(c3ccccc3)C2)cn2nc(C(=O)NC3CCC(c4cccc(C(=O)O)n4)CC3)nc12] |
| 230 | 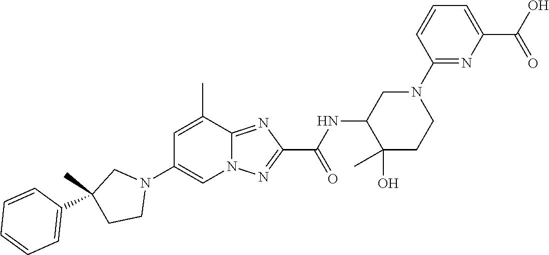 [SMILES: Cc1cc(N2CC[C@](C)(c3ccccc3)C2)cn2nc(C(=O)NC3CN(c4cccc(C(=O)O)n4)CCC3(C)O)nc12] |
| 232 | 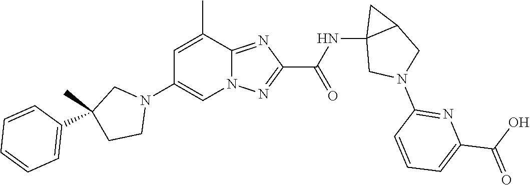 [SMILES: Cc1cc(N2CC[C@](C)(c3ccccc3)C2)cn2nc(C(=O)NC34CC3CN(c3cccc(C(=O)O)n3)C4)nc12] |
| 237 | 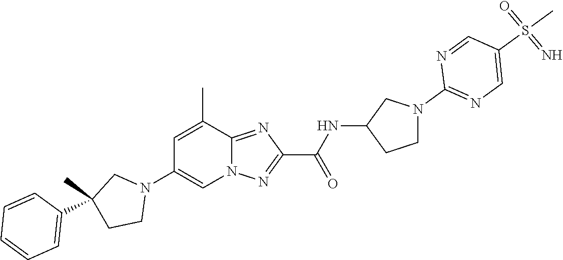 [SMILES: Cc1cc(N2CC[C@](C)(c3ccccc3)C2)cn2nc(C(=O)NC3CCN(c4ncc(S(C)(=N)=O)cn4)C3)nc12] |
| 238 | 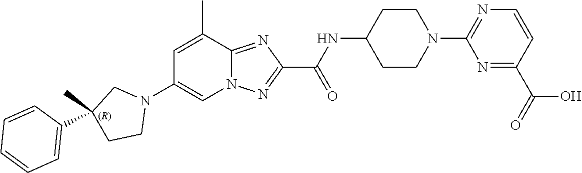 |
| 243 | 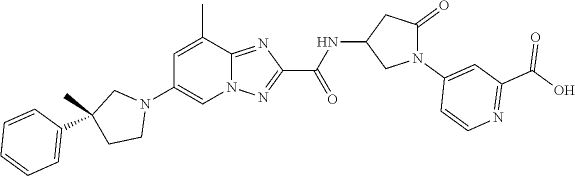 [SMILES: Cc1cc(N2CC[C@](C)(c3ccccc3)C2)cn2nc(C(=O)NC3CC(=O)N(c4ccnc(C(=O)O)c4)C3)nc12] |
| 245 | 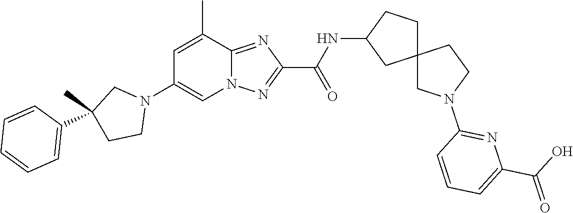 [SMILES: Cc1cc(N2CC[C@](C)(c3ccccc3)C2)cn2nc(C(=O)NC3CCC4(CCN(c5cccc(C(=O)O)n5)C4)C3)nc12] |
| 251 | 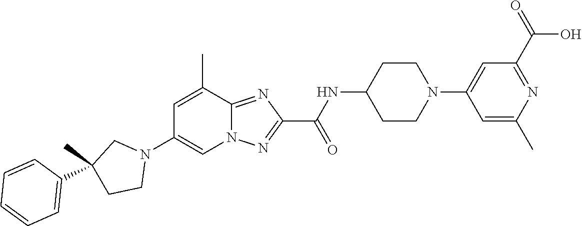 [SMILES: Cc1cc(N2CCC(NC(=O)c3nc4c(C)cc(N5CC[C@](C)(c6ccccc6)C5)cn4n3)CC2)cc(C(=O)O)n1] |
| 254 | 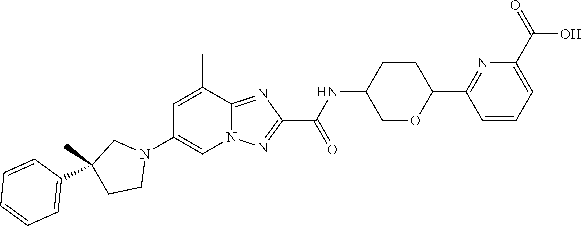 [SMILES: Cc1cc(N2CC[C@](C)(c3ccccc3)C2)cn2nc(C(=O)NC3CCC(c4cccc(C(=O)O)n4)OC3)nc12] |
| 256 | 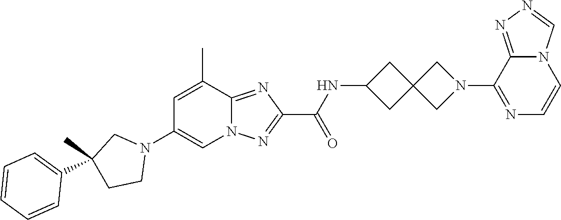 [SMILES: Cc1cc(N2CC[C@](C)(c3ccccc3)C2)cn2nc(C(=O)NC3CC4(C3)CN(c3nccn5cnnc35)C4)nc12] |
| 261 | 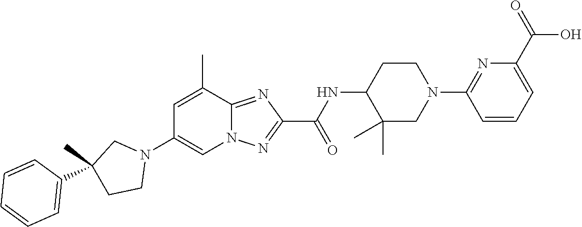 [SMILES: Cc1cc(N2CC[C@](C)(c3ccccc3)C2)cn2nc(C(=O)NC3CCN(c4cccc(C(=O)O)n4)CC3(C)C)nc12] |
| 262 | 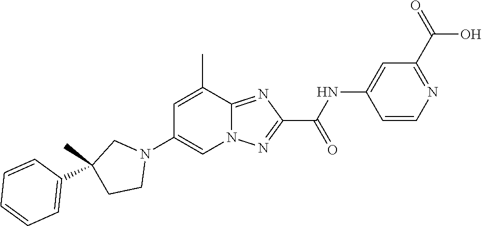 [SMILES: Cc1cc(N2CC[C@](C)(c3ccccc3)C2)cn2nc(C(=O)Nc3ccnc(C(=O)O)c3)nc12] |
| 263 | 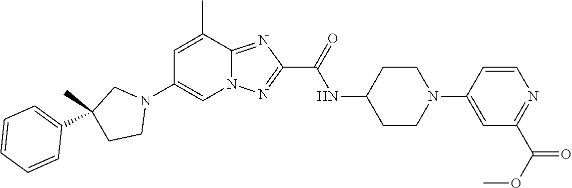 [SMILES: COC(=O)c1cc(N2CCC(NC(=O)c3nc4c(C)cc(N5CC[C@](C)(c6ccccc6)C5)cn4n3)CC2)ccn1] |
| 264 | 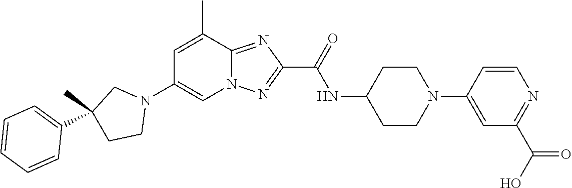 [SMILES: Cc1cc(N2CC[C@](C)(c3ccccc3)C2)cn2nc(C(=O)NC3CCN(c4ccnc(C(=O)O)c4)CC3)nc12] |
| 265 | 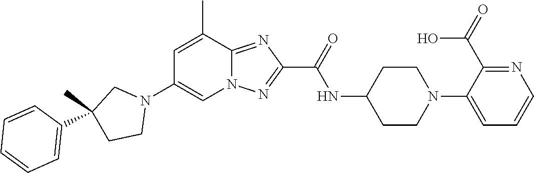 [SMILES: Cc1cc(N2CC[C@](C)(c3ccccc3)C2)cn2nc(C(=O)NC3CCN(c4cccnc4C(=O)O)CC3)nc12] |
| 269 | 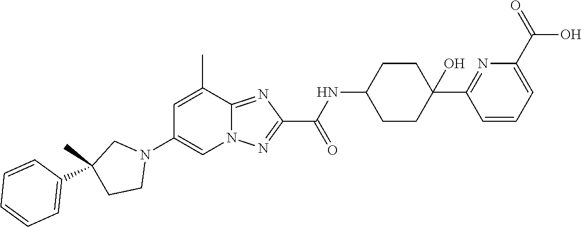 [SMILES: Cc1cc(N2CC[C@](C)(c3ccccc3)C2)cn2nc(C(=O)NC3CCC(O)(c4cccc(C(=O)O)n4)CC3)nc12] |
| 271 | 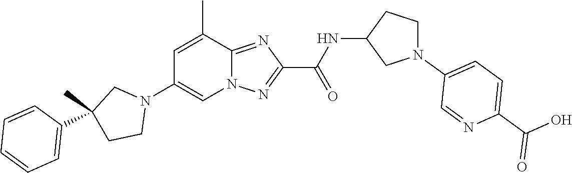 [SMILES: Cc1cc(N2CC[C@](C)(c3ccccc3)C2)cn2nc(C(=O)NC3CCN(c4ccc(C(=O)O)nc4)C3)nc12] |
| 272 | 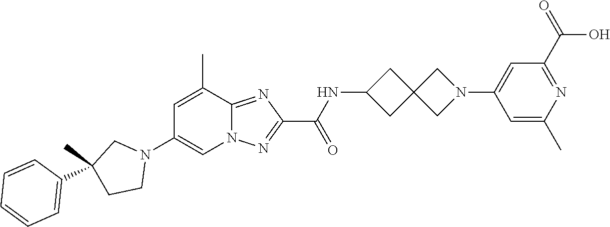 [SMILES: Cc1cc(N2CC3(CC(NC(=O)c4nc5c(C)cc(N6CC[C@](C)(c7ccccc7)C6)cn5n4)C3)C2)cc(C(=O)O)n1] |
| 273 | 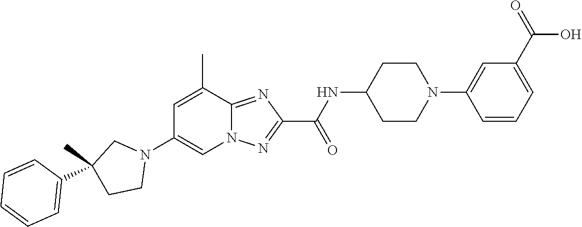 [SMILES: Cc1cc(N2CC[C@](C)(c3ccccc3)C2)cn2nc(C(=O)NC3CCN(c4cccc(C(=O)O)c4)CC3)nc12] |
| 274 | 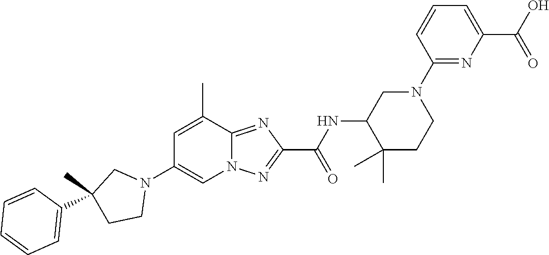 [SMILES: Cc1cc(N2CC[C@](C)(c3ccccc3)C2)cn2nc(C(=O)NC3CN(c4cccc(C(=O)O)n4)CCC3(C)C)nc12] |
| 276 | 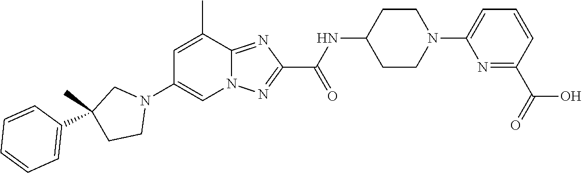 [SMILES: Cc1cc(N2CC[C@](C)(c3ccccc3)C2)cn2nc(C(=O)NC3CCN(c4cccc(C(=O)O)n4)CC3)nc12] |
| 277 | 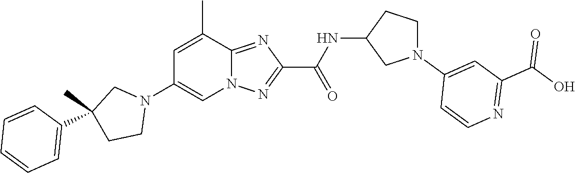 [SMILES: Cc1cc(N2CC[C@](C)(c3ccccc3)C2)cn2nc(C(=O)NC3CCN(c4ccnc(C(=O)O)c4)C3)nc12] |
| 282 | 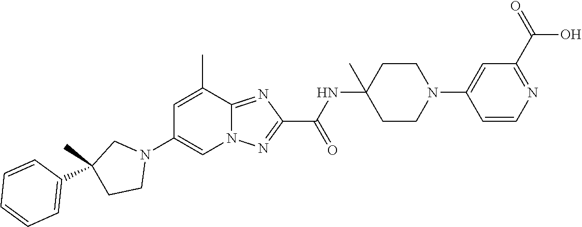 [SMILES: Cc1cc(N2CC[C@](C)(c3ccccc3)C2)cn2nc(C(=O)NC3(C)CCN(c4ccnc(C(=O)O)c4)CC3)nc12] |
| 283 | 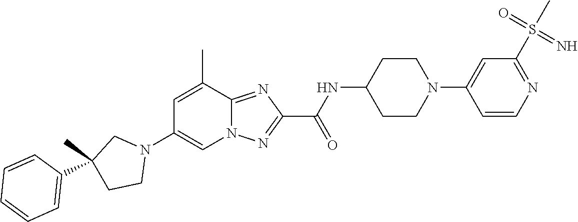 [SMILES: Cc1cc(N2CC[C@](C)(c3ccccc3)C2)cn2nc(C(=O)NC3CCN(c4ccnc(S(C)(=N)=O)c4)CC3)nc12] |
| 284 | 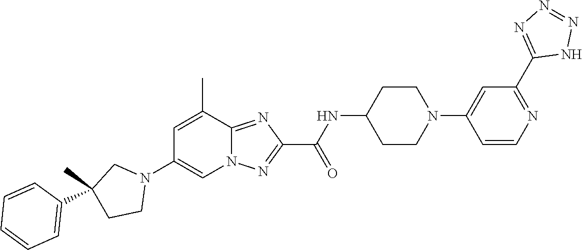 [SMILES: Cc1cc(N2CC[C@](C)(c3ccccc3)C2)cn2nc(C(=O)NC3CCN(c4ccnc(-c5nnn[nH]5)c4)CC3)nc12] |
| 285 | 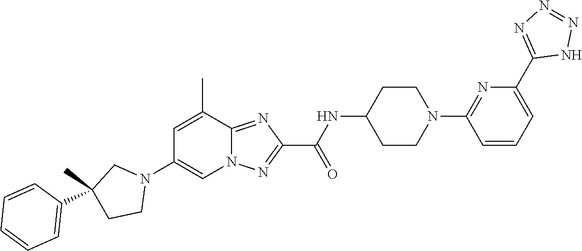 [SMILES: Cc1cc(N2CC[C@](C)(c3ccccc3)C2)cn2nc(C(=O)NC3CCN(c4cccc(-c5nnn[nH]5)n4)CC3)nc12] |
| 290 | 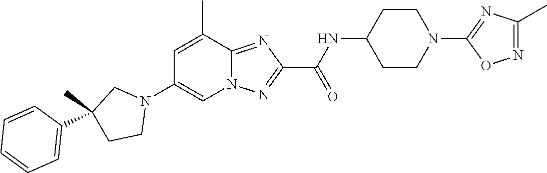 [SMILES: Cc1noc(N2CCC(NC(=O)c3nc4c(C)cc(N5CC[C@](C)(c6ccccc6)C5)cn4n3)CC2)n1] |
| 20 | 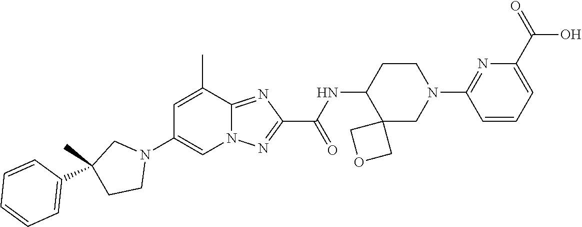 [SMILES: Cc1cc(N2CC[C@](C)(c3ccccc3)C2)cn2nc(C(=O)NC3CCN(c4cccc(C(=O)O)n4)CC34COC4)nc12] |
| 183 | 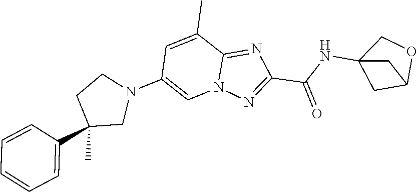 [SMILES: Cc1cc(N2CC[C@](C)(c3ccccc3)C2)cn2nc(C(=O)NC34COC(C3)C4)nc12] |
| 28 | 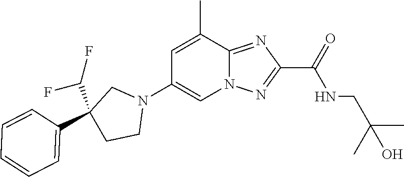 [SMILES: Cc1cc(N2CC[C@](c3ccccc3)(C(F)F)C2)cn2nc(C(=O)NCC(C)(C)O)nc12] |
| 327 | 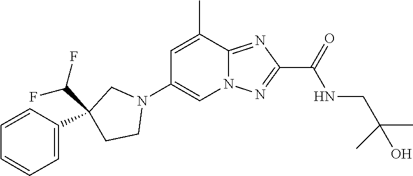 [SMILES: Cc1cc(N2CC[C@@](c3ccccc3)(C(F)F)C2)cn2nc(C(=O)NCC(C)(C)O)nc12] |
| 328 | 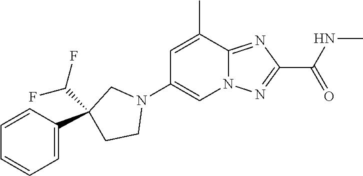 [SMILES: CNC(=O)c1nc2c(C)cc(N3CC[C@](c4ccccc4)(C(F)F)C3)cn2n1] |
| 329 | 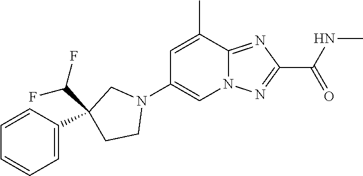 [SMILES: CNC(=O)c1nc2c(C)cc(N3CC[C@@](c4ccccc4)(C(F)F)C3)cn2n1] |
| 330 | 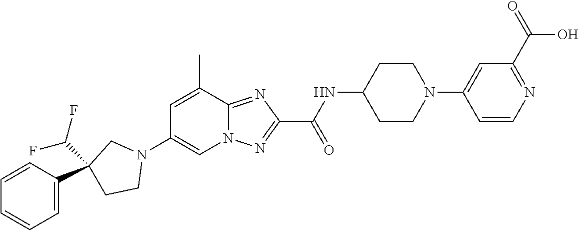 [SMILES: Cc1cc(N2CC[C@](c3ccccc3)(C(F)F)C2)cn2nc(C(=O)NC3CCN(c4ccnc(C(=O)O)c4)CC3)nc12] |
| 30 | 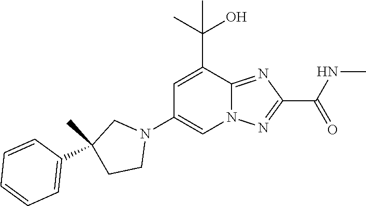 [SMILES: CNC(=O)c1nc2c(C(C)(C)O)cc(N3CC[C@](C)(c4ccccc4)C3)cn2n1] |
| 335 | 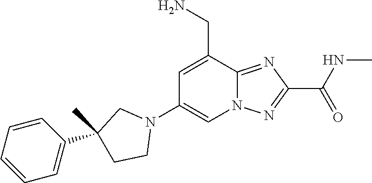 [SMILES: CNC(=O)c1nc2c(CN)cc(N3CC[C@](C)(c4ccccc4)C3)cn2n1] |
| 336 | 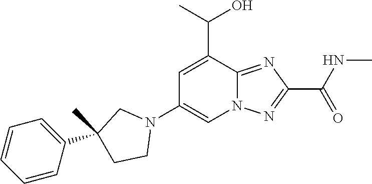 [SMILES: CNC(=O)c1nc2c(C(C)O)cc(N3CC[C@](C)(c4ccccc4)C3)cn2n1] |
| 337 | 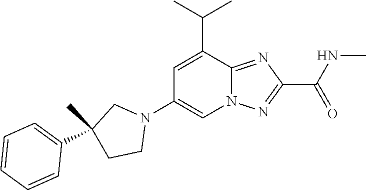 [SMILES: CNC(=O)c1nc2c(C(C)C)cc(N3CC[C@](C)(c4ccccc4)C3)cn2n1] |
| 338 | 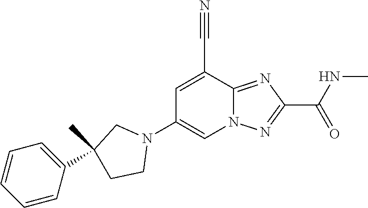 [SMILES: CNC(=O)c1nc2c(C#N)cc(N3CC[C@](C)(c4ccccc4)C3)cn2n1] |
| 339 | 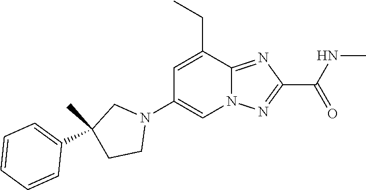 [SMILES: CCc1cc(N2CC[C@](C)(c3ccccc3)C2)cn2nc(C(=O)NC)nc12] |
| 340 | 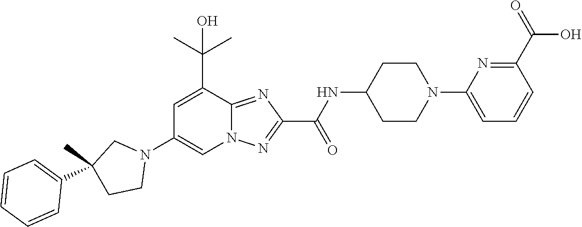 [SMILES: CC(C)(O)c1cc(N2CC[C@](C)(c3ccccc3)C2)cn2nc(C(=O)NC3CCN(c4cccc(C(=O)O)n4)CC3)nc12] |
| 341 | 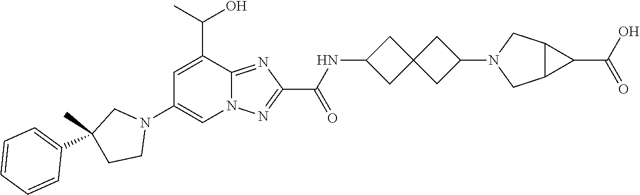 [SMILES: CC(O)c1cc(N2CC[C@](C)(c3ccccc3)C2)cn2nc(C(=O)NC3CC4(C3)CC(N3CC5C(C3)C5C(=O)O)C4)nc12] |
| 342 | 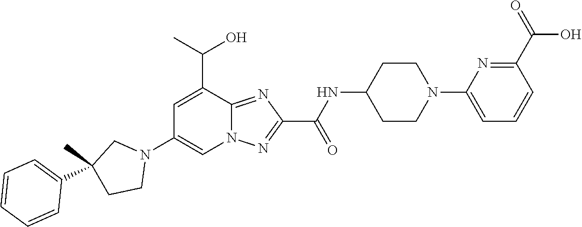 [SMILES: CC(O)c1cc(N2CC[C@](C)(c3ccccc3)C2)cn2nc(C(=O)NC3CCN(c4cccc(C(=O)O)n4)CC3)nc12] |
| 346 | 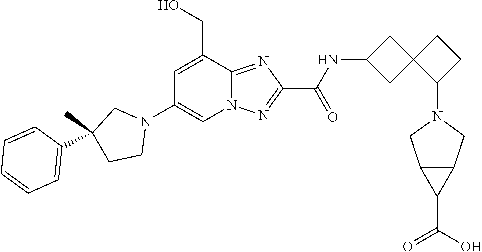 [SMILES: C[C@]1(c2ccccc2)CCN(c2cc(CO)c3nc(C(=O)NC4CC5(CCC5N5CC6C(C5)C6C(=O)O)C4)nn3c2)C1] |
| 348 | 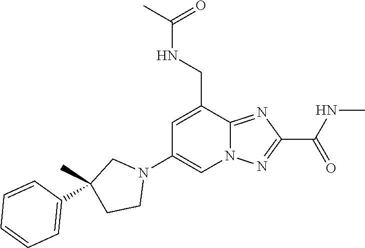 [SMILES: CNC(=O)c1nc2c(CNC(C)=O)cc(N3CC[C@](C)(c4ccccc4)C3)cn2n1] |
| 349 | 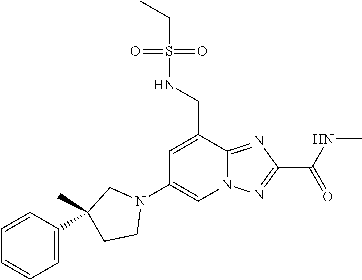 [SMILES: CCS(=O)(=O)NCc1cc(N2CC[C@](C)(c3ccccc3)C2)cn2nc(C(=O)NC)nc12] |
| 350 | 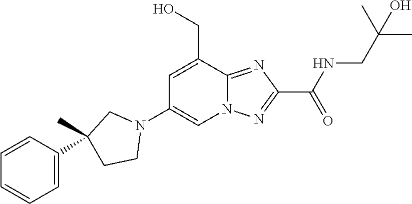 [SMILES: CC(C)(O)CNC(=O)c1nc2c(CO)cc(N3CC[C@](C)(c4ccccc4)C3)cn2n1] |
| 351 | 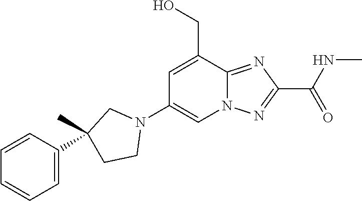 [SMILES: CNC(=O)c1nc2c(CO)cc(N3CC[C@](C)(c4ccccc4)C3)cn2n1] |
| 352 | 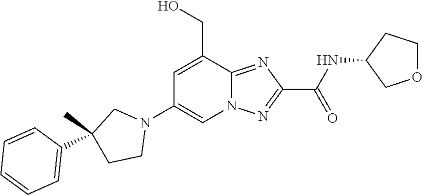 [SMILES: C[C@]1(c2ccccc2)CCN(c2cc(CO)c3nc(C(=O)N[C@@H]4CCOC4)nn3c2)C1] |
| 31 | 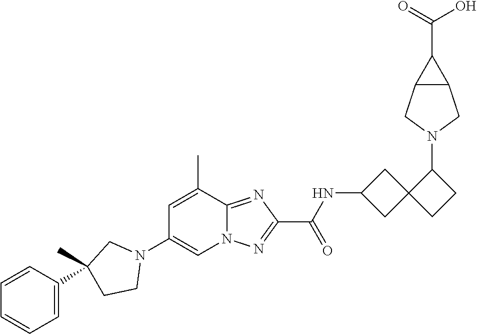 [SMILES: Cc1cc(N2CC[C@](C)(c3ccccc3)C2)cn2nc(C(=O)NC3CC4(CCC4N4CC5C(C4)C5C(=O)O)C3)nc12] |
| 354 | 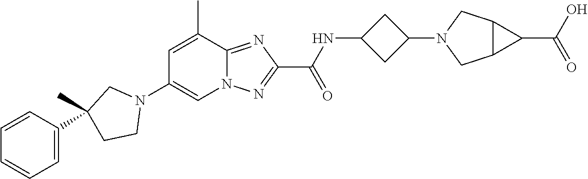 [SMILES: Cc1cc(N2CC[C@](C)(c3ccccc3)C2)cn2nc(C(=O)NC3CC(N4CC5C(C4)C5C(=O)O)C3)nc12] |
| 356 | 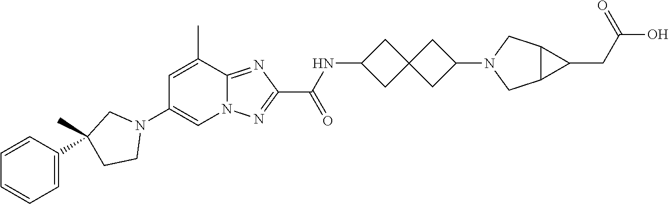 [SMILES: Cc1cc(N2CC[C@](C)(c3ccccc3)C2)cn2nc(C(=O)NC3CC4(C3)CC(N3CC5C(CC(=O)O)C5C3)C4)nc12] |
| 357 | 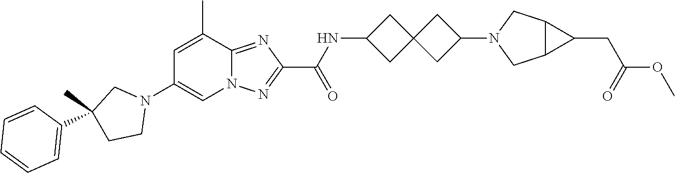 [SMILES: COC(=O)CC1C2CN(C3CC4(CC(NC(=O)c5nc6c(C)cc(N7CC[C@](C)(c8ccccc8)C7)cn6n5)C4)C3)CC12] |
| 358 | 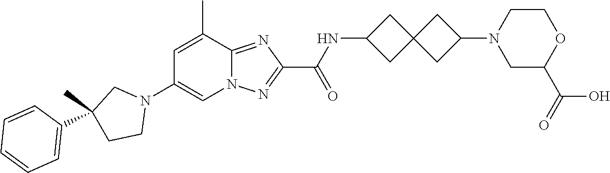 [SMILES: Cc1cc(N2CC[C@](C)(c3ccccc3)C2)cn2nc(C(=O)NC3CC4(C3)CC(N3CCOC(C(=O)O)C3)C4)nc12] |
| 359 | 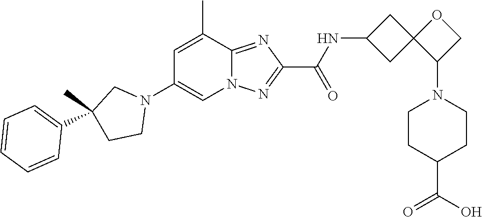 [SMILES: Cc1cc(N2CC[C@](C)(c3ccccc3)C2)cn2nc(C(=O)NC3CC4(C3)OCC4N3CCC(C(=O)O)CC3)nc12] |
| 360 | 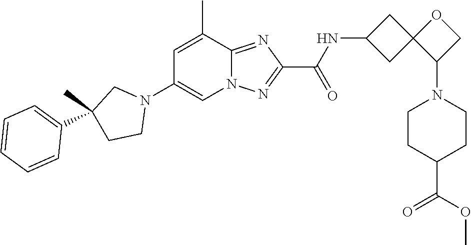 [SMILES: COC(=O)C1CCN(C2COC23CC(NC(=O)c2nc4c(C)cc(N5CC[C@](C)(c6ccccc6)C5)cn4n2)C3)CC1] |
| 361 | 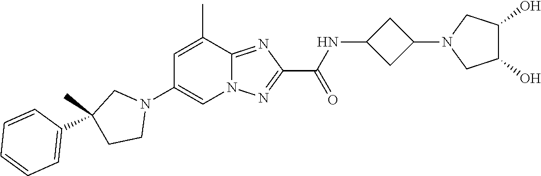 [SMILES: Cc1cc(N2CC[C@](C)(c3ccccc3)C2)cn2nc(C(=O)NC3CC(N4C[C@@H](O)[C@@H](O)C4)C3)nc12] |
| 362 | 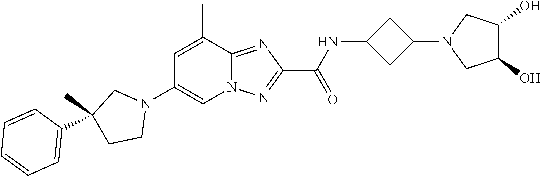 [SMILES: Cc1cc(N2CC[C@](C)(c3ccccc3)C2)cn2nc(C(=O)NC3CC(N4C[C@H](O)[C@@H](O)C4)C3)nc12] |
| 363 | 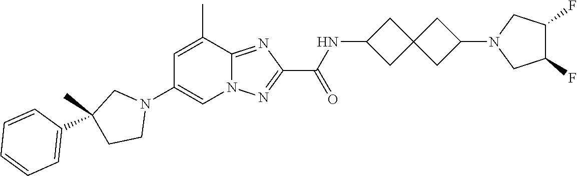 [SMILES: Cc1cc(N2CC[C@](C)(c3ccccc3)C2)cn2nc(C(=O)NC3CC4(C3)CC(N3C[C@H](F)[C@@H](F)C3)C4)nc12] |
| 364 | 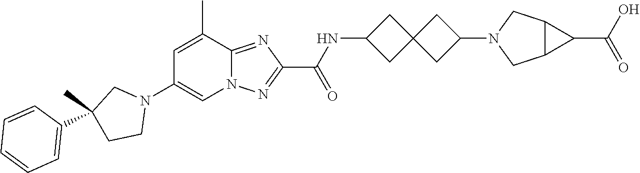 [SMILES: Cc1cc(N2CC[C@](C)(c3ccccc3)C2)cn2nc(C(=O)NC3CC4(C3)CC(N3CC5C(C3)C5C(=O)O)C4)nc12] |
| 365 | 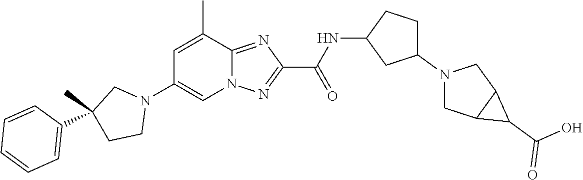 [SMILES: Cc1cc(N2CC[C@](C)(c3ccccc3)C2)cn2nc(C(=O)NC3CCC(N4CC5C(C4)C5C(=O)O)C3)nc12] |
| 367 | 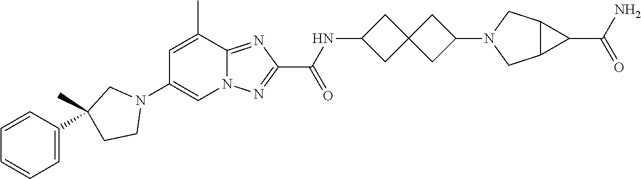 [SMILES: Cc1cc(N2CC[C@](C)(c3ccccc3)C2)cn2nc(C(=O)NC3CC4(C3)CC(N3CC5C(C3)C5C(N)=O)C4)nc12] |
| 368 | 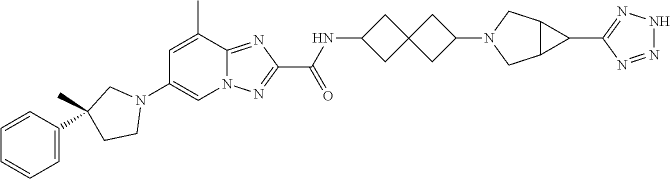 [SMILES: Cc1cc(N2CC[C@](C)(c3ccccc3)C2)cn2nc(C(=O)NC3CC4(C3)CC(N3CC5C(C3)C5c3nn[nH]n3)C4)nc12] |
| 369 | 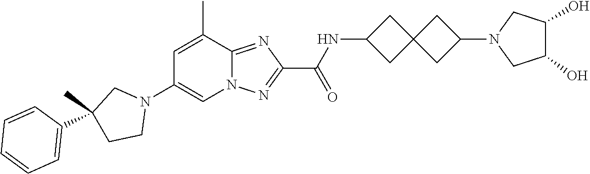 [SMILES: Cc1cc(N2CC[C@](C)(c3ccccc3)C2)cn2nc(C(=O)NC3CC4(C3)CC(N3C[C@@H](O)[C@@H](O)C3)C4)nc12] |
| 371 | 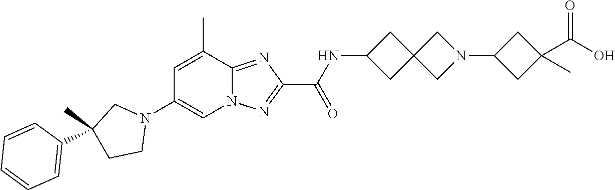 [SMILES: Cc1cc(N2CC[C@](C)(c3ccccc3)C2)cn2nc(C(=O)NC3CC4(C3)CN(C3CC(C)(C(=O)O)C3)C4)nc12] |
| 372 | 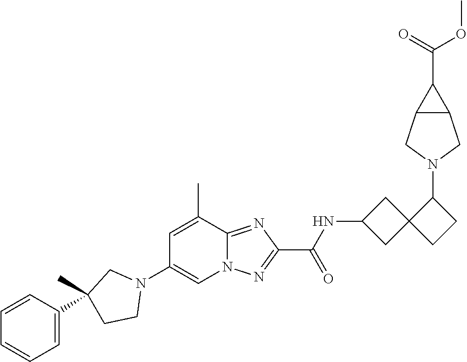 [SMILES: COC(=O)C1C2CN(C3CCC34CC(NC(=O)c3nc5c(C)cc(N6CC[C@](C)(c7ccccc7)C6)cn5n3)C4)CC21] |
| 373 | 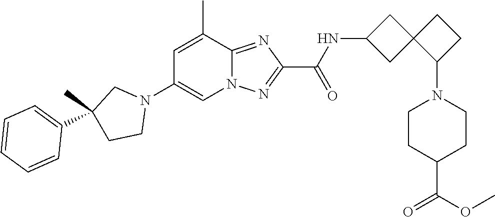 [SMILES: COC(=O)C1CCN(C2CCC23CC(NC(=O)c2nc4c(C)cc(N5CC[C@](C)(c6ccccc6)C5)cn4n2)C3)CC1] |
| 374 | 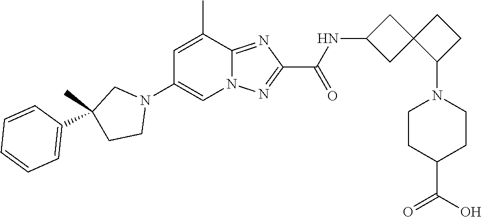 [SMILES: Cc1cc(N2CC[C@](C)(c3ccccc3)C2)cn2nc(C(=O)NC3CC4(CCC4N4CCC(C(=O)O)CC4)C3)nc12] |
| 378 | 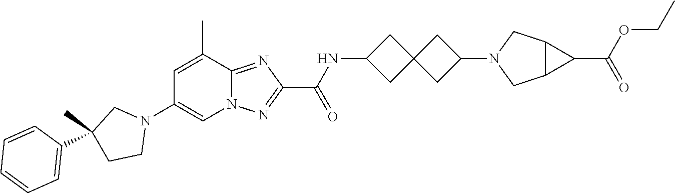 [SMILES: CCOC(=O)C1C2CN(C3CC4(CC(NC(=O)c5nc6c(C)cc(N7CC[C@](C)(c8ccccc8)C7)cn6n5)C4)C3)CC21] |
| 379 | 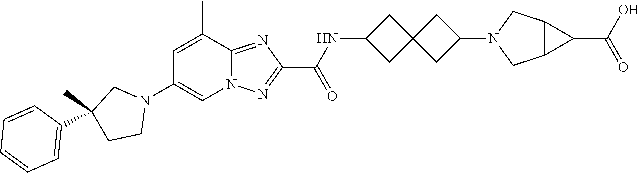 [SMILES: Cc1cc(N2CC[C@](C)(c3ccccc3)C2)cn2nc(C(=O)NC3CC4(C3)CC(N3CC5C(C3)C5C(=O)O)C4)nc12] |
| 380 | 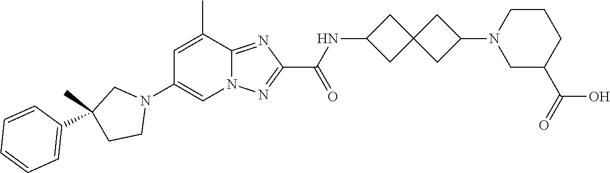 [SMILES: Cc1cc(N2CC[C@](C)(c3ccccc3)C2)cn2nc(C(=O)NC3CC4(C3)CC(N3CCCC(C(=O)O)C3)C4)nc12] |
| 32 | 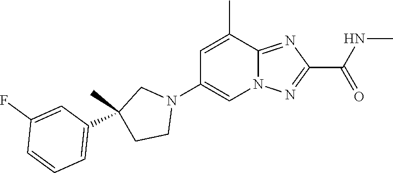 [SMILES: CNC(=O)c1nc2c(C)cc(N3CC[C@](C)(c4cccc(F)c4)C3)cn2n1] |
| 382 | 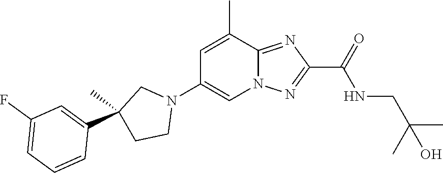 [SMILES: Cc1cc(N2CC[C@@](C)(c3cccc(F)c3)C2)cn2nc(C(=O)NCC(C)(C)O)nc12] |
| 383 | 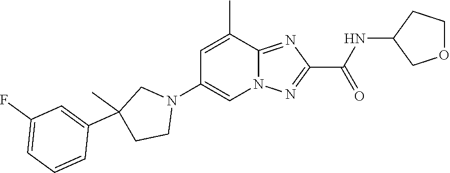 [SMILES: Cc1cc(N2CCC(C)(c3cccc(F)c3)C2)cn2nc(C(=O)NC3CCOC3)nc12] |
| 384 | 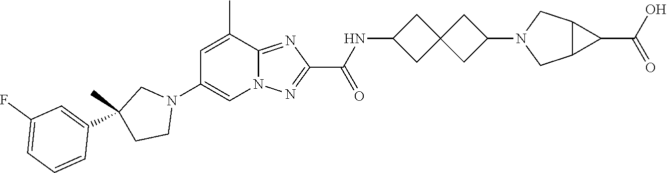 [SMILES: Cc1cc(N2CC[C@](C)(c3cccc(F)c3)C2)cn2nc(C(=O)NC3CC4(C3)CC(N3CC5C(C3)C5C(=O)O)C4)nc12] |
| 33 | 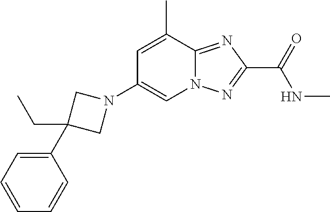 [SMILES: CCC1(c2ccccc2)CN(c2cc(C)c3nc(C(=O)NC)nn3c2)C1] |
| 390 | 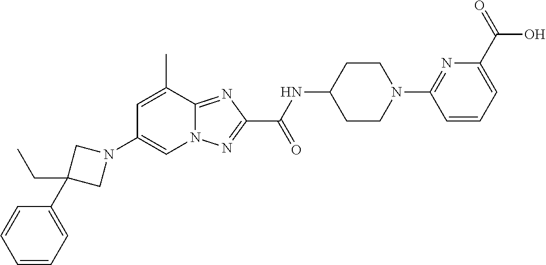 [SMILES: CCC1(c2ccccc2)CN(c2cc(C)c3nc(C(=O)NC4CCN(c5cccc(C(=O)O)n5)CC4)nn3c2)C1] |
| 391 | 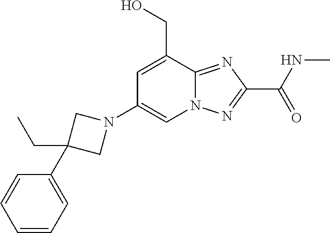 [SMILES: CCC1(c2ccccc2)CN(c2cc(CO)c3nc(C(=O)NC)nn3c2)C1] |
| 392 | 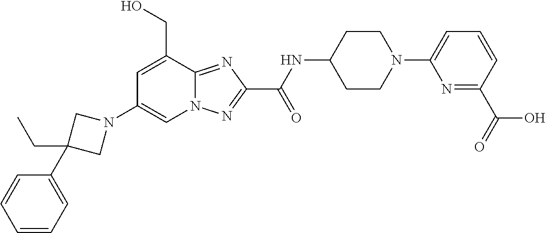 [SMILES: CCC1(c2ccccc2)CN(c2cc(CO)c3nc(C(=O)NC4CCN(c5cccc(C(=O)O)n5)CC4)nn3c2)C1] |
| 395 | 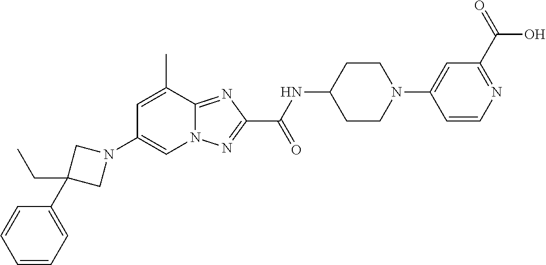 [SMILES: CCC1(c2ccccc2)CN(c2cc(C)c3nc(C(=O)NC4CCN(c5ccnc(C(=O)O)c5)CC4)nn3c2)C1] |
| 34 | 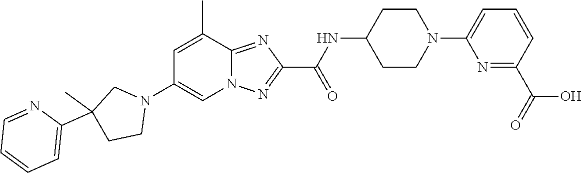 [SMILES: Cc1cc(N2CCC(C)(c3ccccn3)C2)cn2nc(C(=O)NC3CCN(c4cccc(C(=O)O)n4)CC3)nc12] |
| 422 | 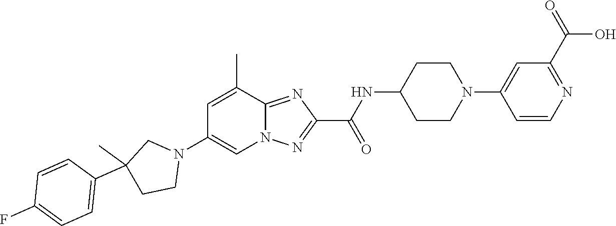 [SMILES: Cc1cc(N2CCC(C)(c3ccc(F)cc3)C2)cn2nc(C(=O)NC3CCN(c4ccnc(C(=O)O)c4)CC3)nc12] |
| 425 | 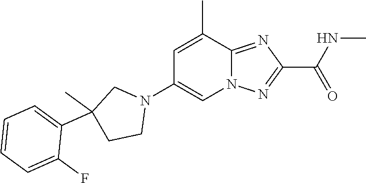 [SMILES: CNC(=O)c1nc2c(C)cc(N3CCC(C)(c4ccccc4F)C3)cn2n1] |
| 591 | 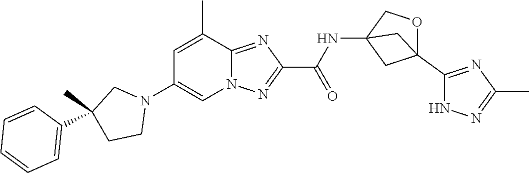 [SMILES: Cc1n[nH]c(C23CC(NC(=O)c4nc5c(C)cc(N6CC[C@](C)(c7ccccc7)C6)cn5n4)(CO2)C3)n1] |
| 593 | 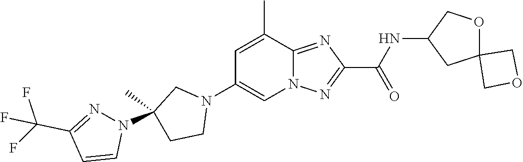 [SMILES: Cc1cc(N2CC[C@@](C)(n3ccc(C(F)(F)F)n3)C2)cn2nc(C(=O)NC3COC4(COC4)C3)nc12] |
| 596 | 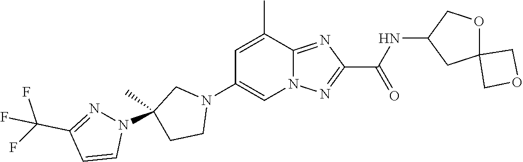 [SMILES: Cc1cc(N2CC[C@@](C)(n3ccc(C(F)(F)F)n3)C2)cn2nc(C(=O)NC3COC4(COC4)C3)nc12] |
| 597 | 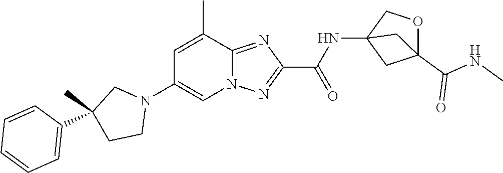 [SMILES: CNC(=O)C12CC(NC(=O)c3nc4c(C)cc(N5CC[C@](C)(c6ccccc6)C5)cn4n3)(CO1)C2] |
| 599 | 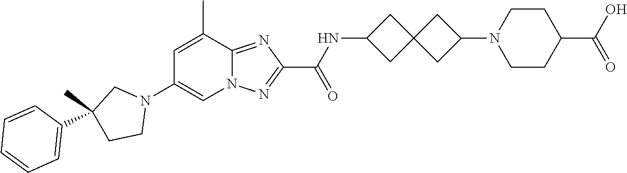 [SMILES: Cc1cc(N2CC[C@](C)(c3ccccc3)C2)cn2nc(C(=O)NC3CC4(C3)CC(N3CCC(C(=O)O)CC3)C4)nc12] |
| 600 | 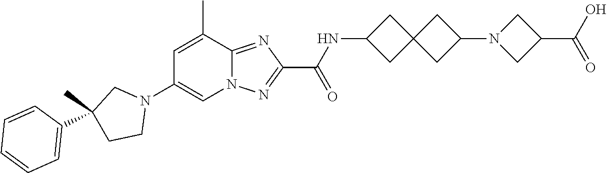 [SMILES: Cc1cc(N2CC[C@](C)(c3ccccc3)C2)cn2nc(C(=O)NC3CC4(C3)CC(N3CC(C(=O)O)C3)C4)nc12] |
| 601 | 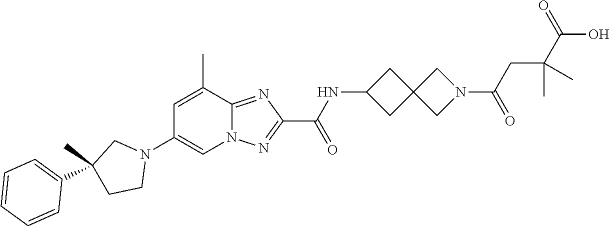 [SMILES: Cc1cc(N2CC[C@](C)(c3ccccc3)C2)cn2nc(C(=O)NC3CC4(C3)CN(C(=O)CC(C)(C)C(=O)O)C4)nc12] |
| 602 | 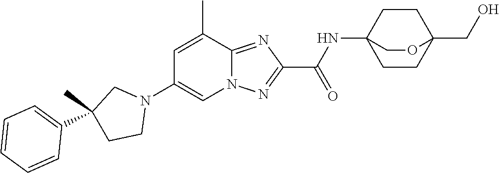 [SMILES: Cc1cc(N2CC[C@](C)(c3ccccc3)C2)cn2nc(C(=O)NC34CCC(CO)(CC3)OC4)nc12] |
| 607 | 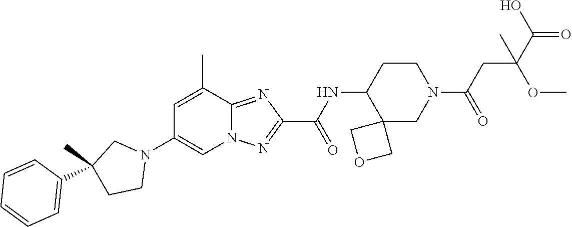 [SMILES: COC(C)(CC(=O)N1CCC(NC(=O)c2nc3c(C)cc(N4CC[C@](C)(c5ccccc5)C4)cn3n2)C2(COC2)C1)C(=O)O] |
| 608 | 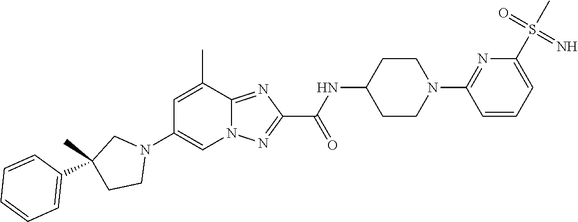 [SMILES: Cc1cc(N2CC[C@](C)(c3ccccc3)C2)cn2nc(C(=O)NC3CCN(c4cccc(S(C)(=N)=O)n4)CC3)nc12] |
| 612 | 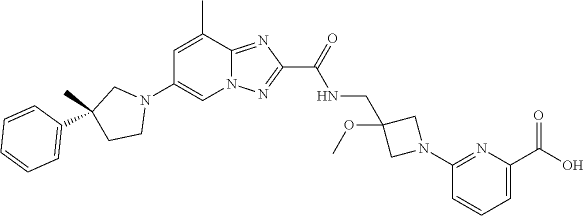 [SMILES: COC1(CNC(=O)c2nc3c(C)cc(N4CC[C@](C)(c5ccccc5)C4)cn3n2)CN(c2cccc(C(=O)O)n2)C1] |
| 613 | 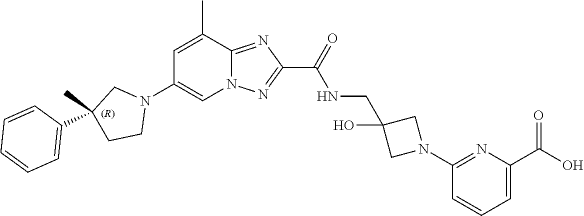 |
| 614 | 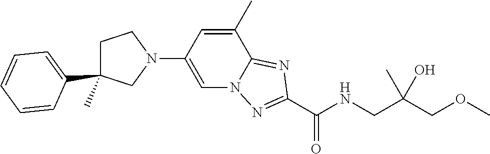 [SMILES: COCC(C)(O)CNC(=O)c1nc2c(C)cc(N3CC[C@](C)(c4ccccc4)C3)cn2n1] |
| 617 | 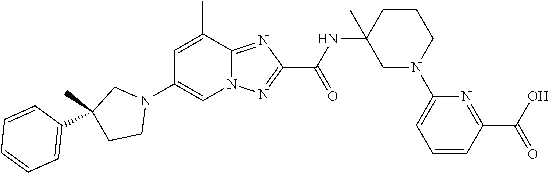 [SMILES: Cc1cc(N2CC[C@](C)(c3ccccc3)C2)cn2nc(C(=O)NC3(C)CCCN(c4cccc(C(=O)O)n4)C3)nc12] |
| 620 | 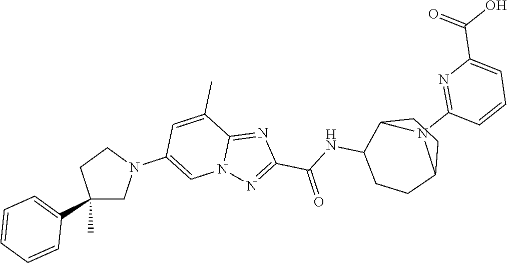 [SMILES: Cc1cc(N2CC[C@](C)(c3ccccc3)C2)cn2nc(C(=O)NC3CCC4CCC3N4c3cccc(C(=O)O)n3)nc12] |
| 623 | 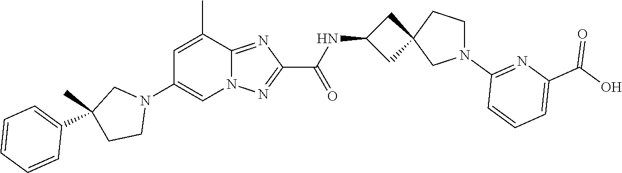 [SMILES: Cc1cc(N2CC[C@](C)(c3ccccc3)C2)cn2nc(C(=O)N[C@H]3C[C@]4(CCN(c5cccc(C(=O)O)n5)C4)C3)nc12] |
| 624 | 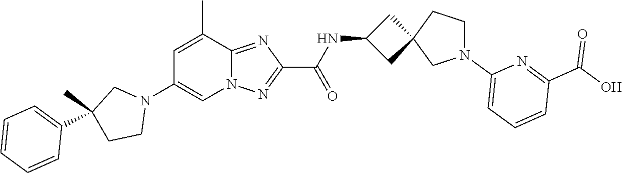 [SMILES: Cc1cc(N2CC[C@](C)(c3ccccc3)C2)cn2nc(C(=O)N[C@H]3C[C@@]4(CCN(c5cccc(C(=O)O)n5)C4)C3)nc12] |
| 628 | 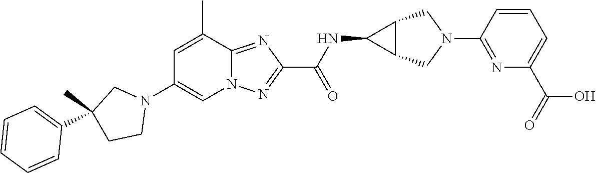 [SMILES: Cc1cc(N2CC[C@](C)(c3ccccc3)C2)cn2nc(C(=O)N[C@H]3[C@@H]4CN(c5cccc(C(=O)O)n5)C[C@@H]43)nc12] |
| 633 | 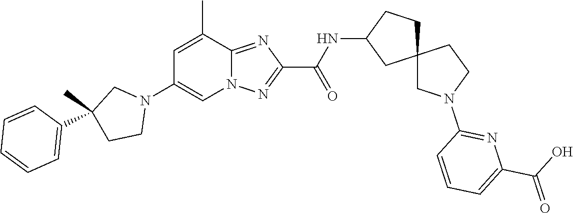 |
| 634 | 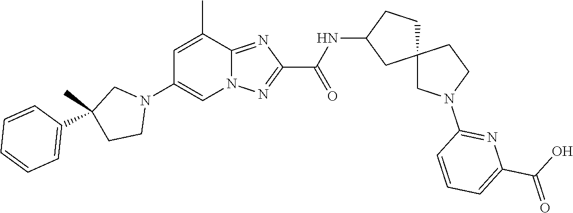 |
| 635 | 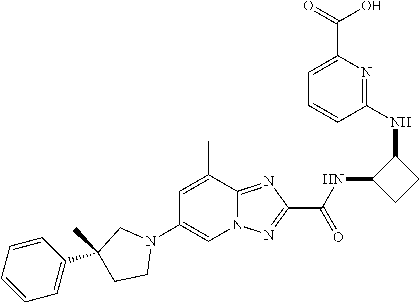 |
| 646 | 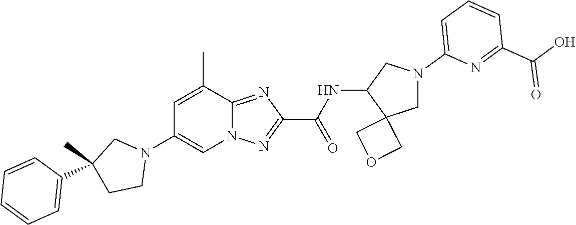 [SMILES: Cc1cc(N2CC[C@](C)(c3ccccc3)C2)cn2nc(C(=O)NC3CN(c4cccc(C(=O)O)n4)CC34COC4)nc12] |
| 647 | 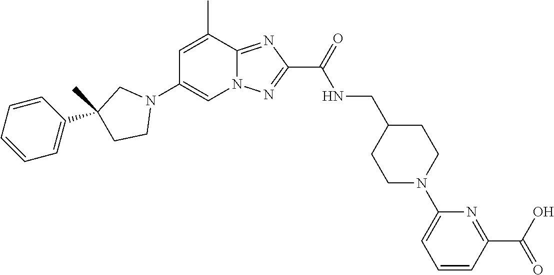 [SMILES: Cc1cc(N2CC[C@](C)(c3ccccc3)C2)cn2nc(C(=O)NCC3CCN(c4cccc(C(=O)O)n4)CC3)nc12] |
| 648 | 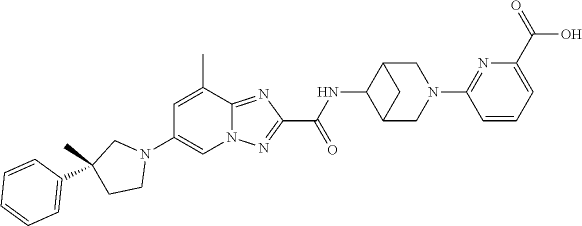 [SMILES: Cc1cc(N2CC[C@](C)(c3ccccc3)C2)cn2nc(C(=O)NC3C4CC3CN(c3cccc(C(=O)O)n3)C4)nc12] |
| 652 | 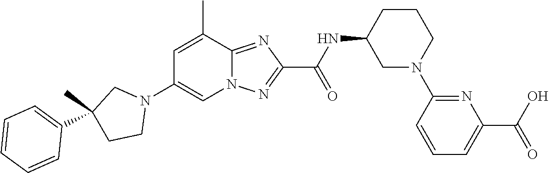 [SMILES: Cc1cc(N2CC[C@](C)(c3ccccc3)C2)cn2nc(C(=O)N[C@H]3CCCN(c4cccc(C(=O)O)n4)C3)nc12] |
| 654 | 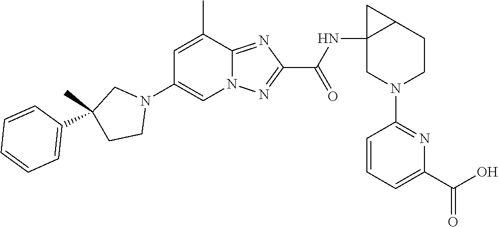 [SMILES: Cc1cc(N2CC[C@](C)(c3ccccc3)C2)cn2nc(C(=O)NC34CC3CCN(c3cccc(C(=O)O)n3)C4)nc12] |
| 655 | 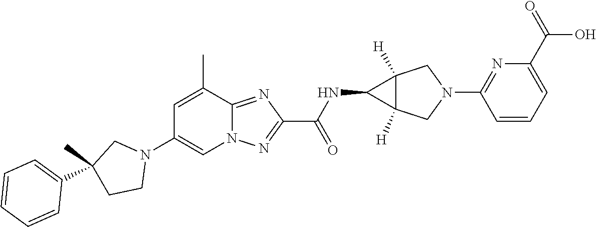 [SMILES: Cc1cc(N2CC[C@](C)(c3ccccc3)C2)cn2nc(C(=O)N[C@@H]3[C@@H]4CN(c5cccc(C(=O)O)n5)C[C@@H]43)nc12] |
| 656 | 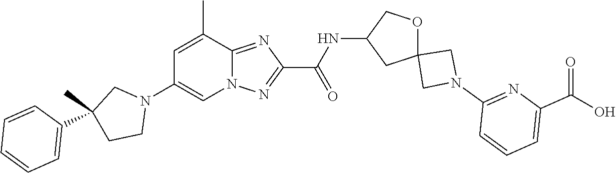 [SMILES: Cc1cc(N2CC[C@](C)(c3ccccc3)C2)cn2nc(C(=O)NC3COC4(C3)CN(c3cccc(C(=O)O)n3)C4)nc12] |
| 658 | 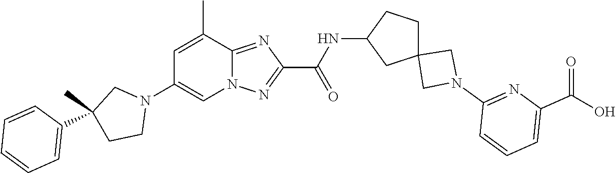 [SMILES: Cc1cc(N2CC[C@](C)(c3ccccc3)C2)cn2nc(C(=O)NC3CCC4(C3)CN(c3cccc(C(=O)O)n3)C4)nc12] |
| 660 | 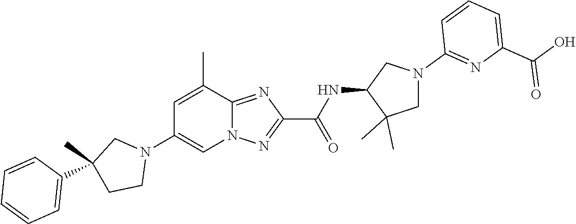 [SMILES: Cc1cc(N2CC[C@](C)(c3ccccc3)C2)cn2nc(C(=O)N[C@H]3CN(c4cccc(C(=O)O)n4)CC3(C)C)nc12] |
| 661 | 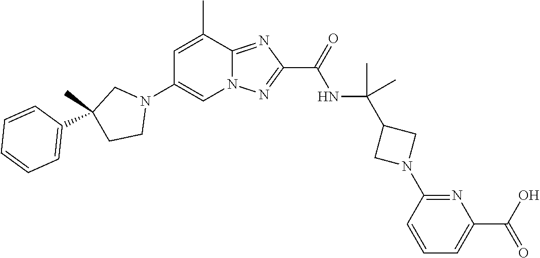 [SMILES: Cc1cc(N2CC[C@](C)(c3ccccc3)C2)cn2nc(C(=O)NC(C)(C)C3CN(c4cccc(C(=O)O)n4)C3)nc12] |
| 662 | 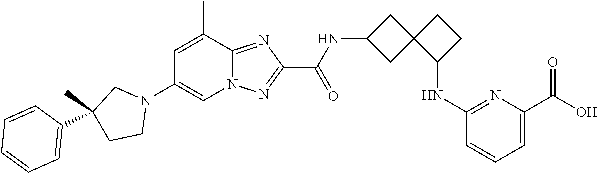 [SMILES: Cc1cc(N2CC[C@](C)(c3ccccc3)C2)cn2nc(C(=O)NC3CC4(CCC4Nc4cccc(C(=O)O)n4)C3)nc12] |
| 663 | 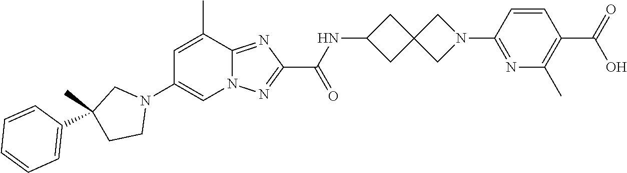 [SMILES: Cc1nc(N2CC3(CC(NC(=O)c4nc5c(C)cc(N6CC[C@](C)(c7ccccc7)C6)cn5n4)C3)C2)ccc1C(=O)O] |
| 664 | 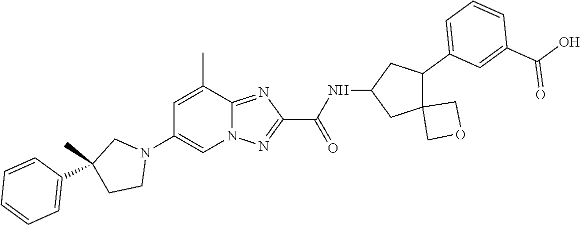 [SMILES: Cc1cc(N2CC[C@](C)(c3ccccc3)C2)cn2nc(C(=O)NC3CC(c4cccc(C(=O)O)c4)C4(COC4)C3)nc12] |
| 666 | 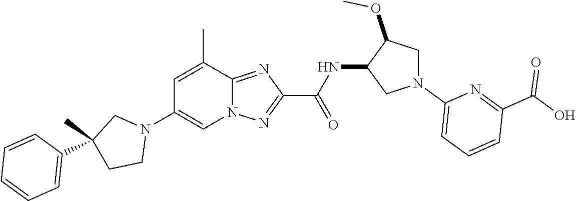 |
| 667 | 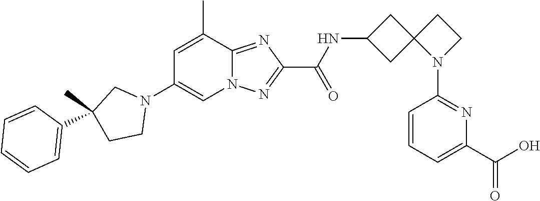 [SMILES: Cc1cc(N2CC[C@](C)(c3ccccc3)C2)cn2nc(C(=O)NC3CC4(CCN4c4cccc(C(=O)O)n4)C3)nc12] |
| 668 | 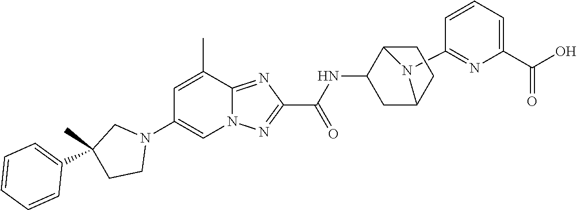 [SMILES: Cc1cc(N2CC[C@](C)(c3ccccc3)C2)cn2nc(C(=O)NC3CC4CCC3N4c3cccc(C(=O)O)n3)nc12] |
| 669 | 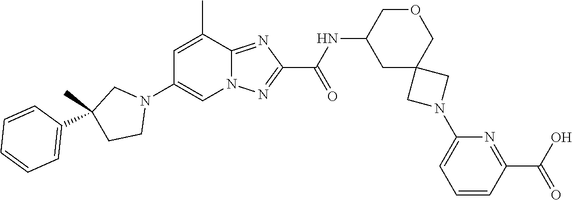 [SMILES: Cc1cc(N2CC[C@](C)(c3ccccc3)C2)cn2nc(C(=O)NC3COCC4(C3)CN(c3cccc(C(=O)O)n3)C4)nc12] |
| 673 | 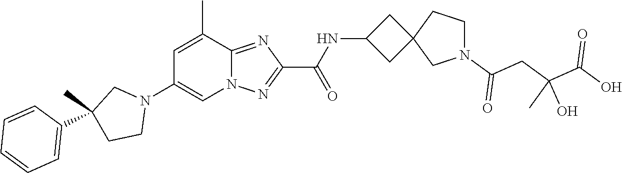 [SMILES: Cc1cc(N2CC[C@](C)(c3ccccc3)C2)cn2nc(C(=O)NC3CC4(CCN(C(=O)CC(C)(O)C(=O)O)C4)C3)nc12] |
| 676 | 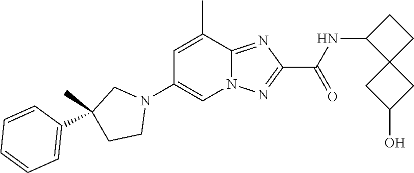 [SMILES: Cc1cc(N2CC[C@](C)(c3ccccc3)C2)cn2nc(C(=O)NC3CCC34CC(O)C4)nc12] |
| 677 | 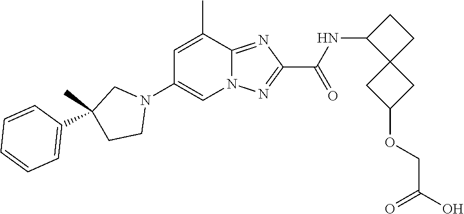 [SMILES: Cc1cc(N2CC[C@](C)(c3ccccc3)C2)cn2nc(C(=O)NC3CCC34CC(OCC(=O)O)C4)nc12] |
| 678 | 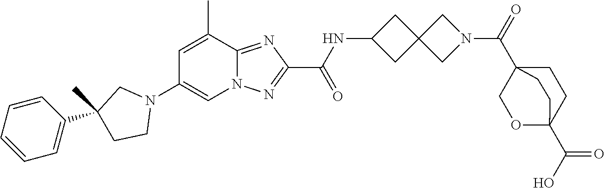 [SMILES: Cc1cc(N2CC[C@](C)(c3ccccc3)C2)cn2nc(C(=O)NC3CC4(C3)CN(C(=O)C35CCC(C(=O)O)(CC3)OC5)C4)nc12] |
| 679 | 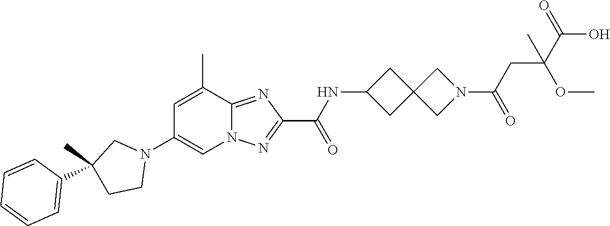 [SMILES: COC(C)(CC(=O)N1CC2(CC(NC(=O)c3nc4c(C)cc(N5CC[C@](C)(c6ccccc6)C5)cn4n3)C2)C1)C(=O)O] |
| 680 | 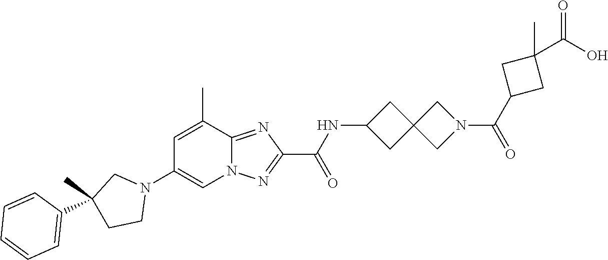 [SMILES: Cc1cc(N2CC[C@](C)(c3ccccc3)C2)cn2nc(C(=O)NC3CC4(C3)CN(C(=O)C3CC(C)(C(=O)O)C3)C4)nc12] |
| 681 | 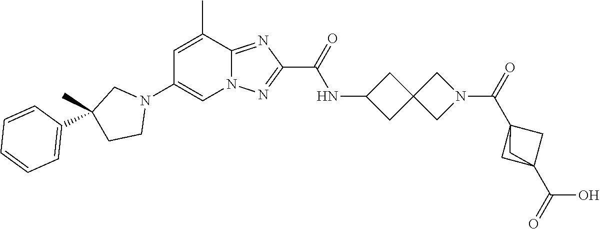 [SMILES: Cc1cc(N2CC[C@](C)(c3ccccc3)C2)cn2nc(C(=O)NC3CC4(C3)CN(C(=O)C35CC(C(=O)O)(C3)C5)C4)nc12] |
| 682 | 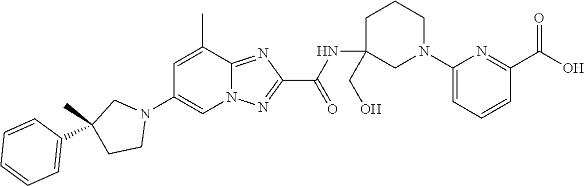 [SMILES: Cc1cc(N2CC[C@](C)(c3ccccc3)C2)cn2nc(C(=O)NC3(CO)CCCN(c4cccc(C(=O)O)n4)C3)nc12] |
| 683 | 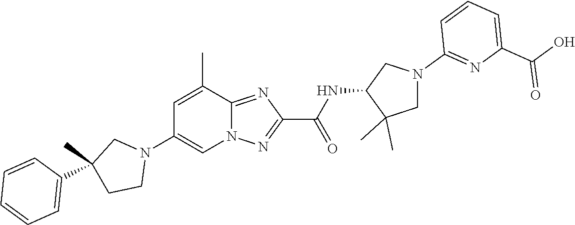 [SMILES: Cc1cc(N2CC[C@](C)(c3ccccc3)C2)cn2nc(C(=O)N[C@@H]3CN(c4cccc(C(=O)O)n4)CC3(C)C)nc12] |
| 684 | 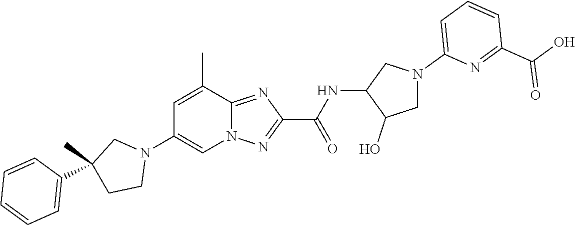 [SMILES: Cc1cc(N2CC[C@](C)(c3ccccc3)C2)cn2nc(C(=O)NC3CN(c4cccc(C(=O)O)n4)CC3O)nc12] |
| 685 | 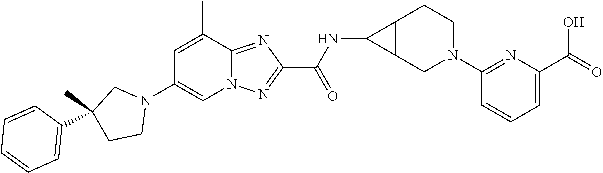 [SMILES: Cc1cc(N2CC[C@](C)(c3ccccc3)C2)cn2nc(C(=O)NC3C4CCN(c5cccc(C(=O)O)n5)CC43)nc12] |
| 689 | 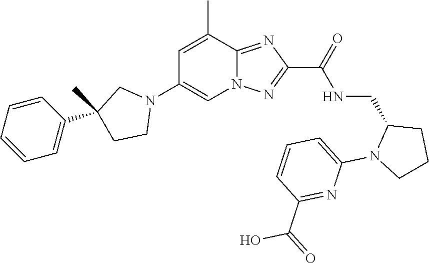 [SMILES: Cc1cc(N2CC[C@](C)(c3ccccc3)C2)cn2nc(C(=O)NC[C@@H]3CCCN3c3cccc(C(=O)O)n3)nc12] |
| 690 | 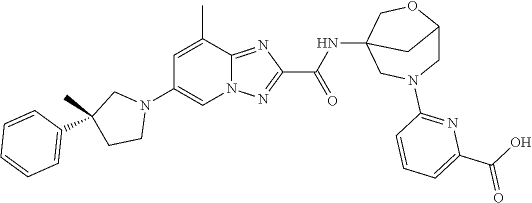 [SMILES: Cc1cc(N2CC[C@](C)(c3ccccc3)C2)cn2nc(C(=O)NC34COC(CN(c5cccc(C(=O)O)n5)C3)C4)nc12] |
| 691 | 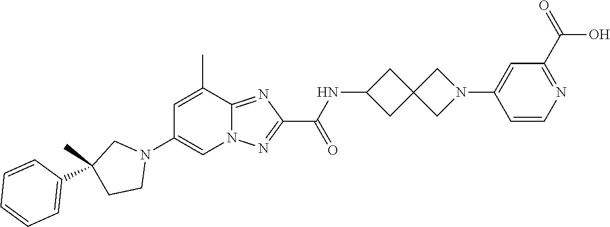 [SMILES: Cc1cc(N2CC[C@](C)(c3ccccc3)C2)cn2nc(C(=O)NC3CC4(C3)CN(c3ccnc(C(=O)O)c3)C4)nc12] |
| 693 | 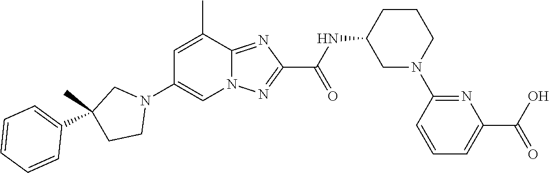 [SMILES: Cc1cc(N2CC[C@](C)(c3ccccc3)C2)cn2nc(C(=O)N[C@@H]3CCCN(c4cccc(C(=O)O)n4)C3)nc12] |
| 697 | 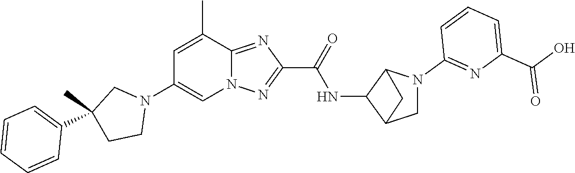 [SMILES: Cc1cc(N2CC[C@](C)(c3ccccc3)C2)cn2nc(C(=O)NC3C4CC3N(c3cccc(C(=O)O)n3)C4)nc12] |
| 698 | 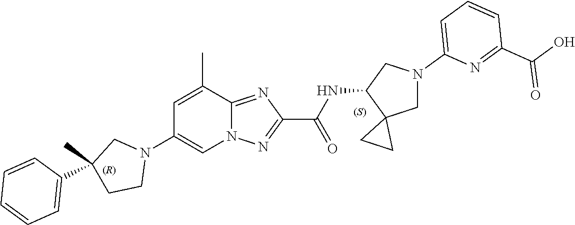 |
| 699 | 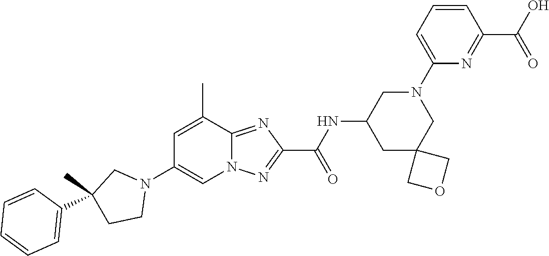 [SMILES: Cc1cc(N2CC[C@](C)(c3ccccc3)C2)cn2nc(C(=O)NC3CN(c4cccc(C(=O)O)n4)CC4(COC4)C3)nc12] |
| 700 | 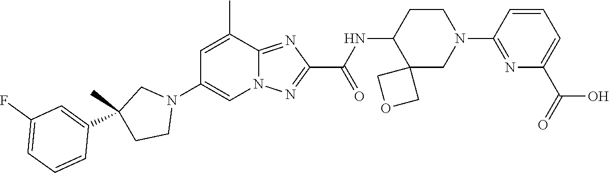 [SMILES: Cc1cc(N2CC[C@](C)(c3cccc(F)c3)C2)cn2nc(C(=O)NC3CCN(c4cccc(C(=O)O)n4)CC34COC4)nc12] |
| 701 | 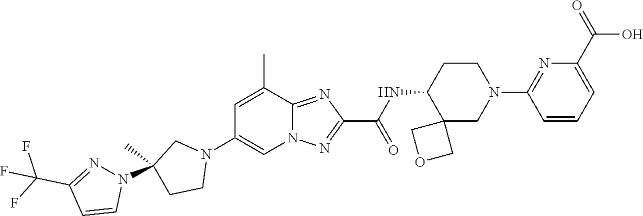 [SMILES: Cc1cc(N2CC[C@@](C)(n3ccc(C(F)(F)F)n3)C2)cn2nc(C(=O)N[C@@H]3CCN(c4cccc(C(=O)O)n4)CC34COC4)nc12] |
| 758 | 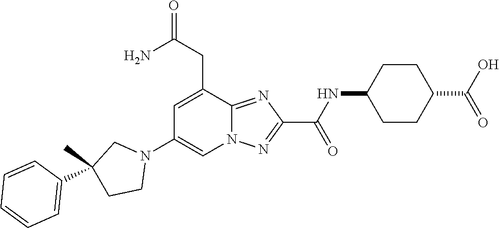 [SMILES: C[C@]1(c2ccccc2)CCN(c2cc(CC(N)=O)c3nc(C(=O)N[C@H]4CC[C@H](C(=O)O)CC4)nn3c2)C1] |
| 760 | 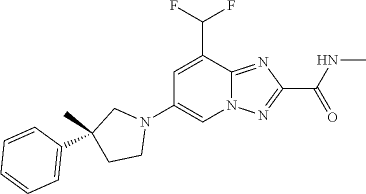 [SMILES: CNC(=O)c1nc2c(C(F)F)cc(N3CC[C@](C)(c4ccccc4)C3)cn2n1] |
| 761 | 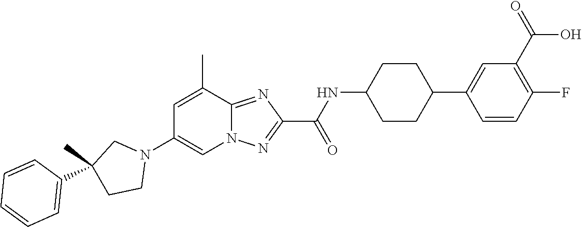 [SMILES: Cc1cc(N2CC[C@](C)(c3ccccc3)C2)cn2nc(C(=O)NC3CCC(c4ccc(F)c(C(=O)O)c4)CC3)nc12] |
| 122 | 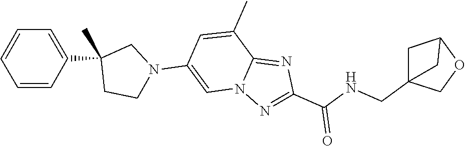 [SMILES: Cc1cc(N2CC[C@](C)(c3ccccc3)C2)cn2nc(C(=O)NCC34COC(C3)C4)nc12] |
| 123 | 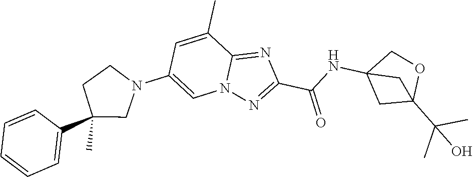 [SMILES: Cc1cc(N2CC[C@](C)(c3ccccc3)C2)cn2nc(C(=O)NC34COC(C(C)(C)O)(C3)C4)nc12] |
| 642 | 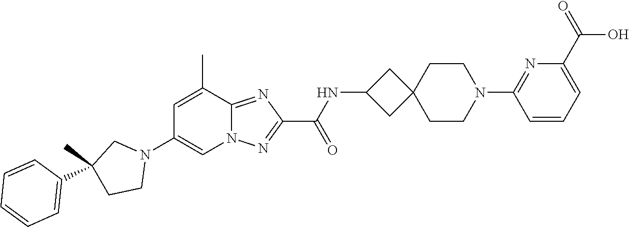 [SMILES: Cc1cc(N2CC[C@](C)(c3ccccc3)C2)cn2nc(C(=O)NC3CC4(CCN(c5cccc(C(=O)O)n5)CC4)C3)nc12] |

TABLE 3

| Cmpd | |
|---|---|
| # | Structure |
| 99 | 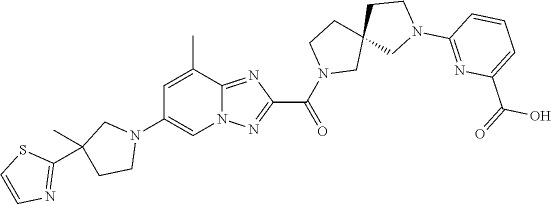 [SMILES: Cc1cc(N2CCC(C)(c3nccs3)C2)cn2nc(C(=O)N3CC[C@@]4(CCN(c5cccc(C(=O)O)n5)C4)C3)nc12] |
| 113 | 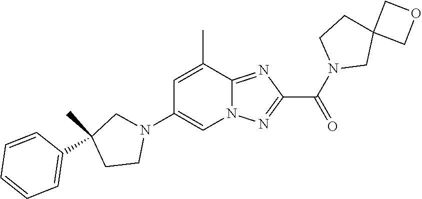 [SMILES: Cc1cc(N2CC[C@](C)(c3ccccc3)C2)cn2nc(C(=O)N3CCC4(COC4)C3)nc12] |
| 115 | 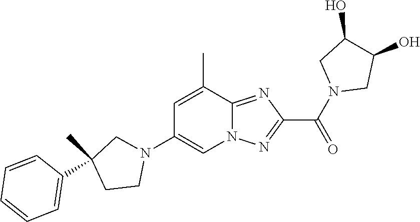 [SMILES: Cc1cc(N2CC[C@](C)(c3ccccc3)C2)cn2nc(C(=O)N3C[C@@H](O)[C@@H](O)C3)nc12] |
| 124 | 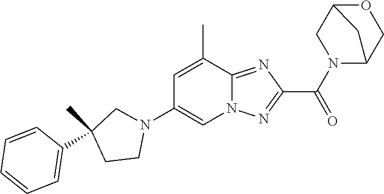 [SMILES: Cc1cc(N2CC[C@](C)(c3ccccc3)C2)cn2nc(C(=O)N3CC4CC3CO4)nc12] |
| 125 | 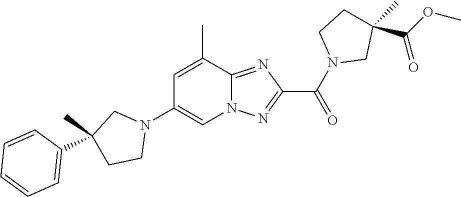 [SMILES: COC(=O)[C@]1(C)CCN(C(=O)c2nc3c(C)cc(N4CC[C@](C)(c5ccccc5)C4)cn3n2)C1] |
| 126 | 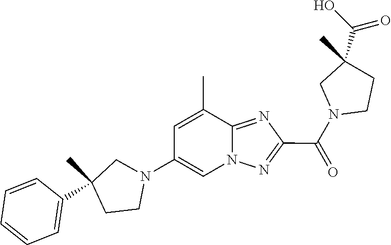 [SMILES: Cc1cc(N2CC[C@](C)(c3ccccc3)C2)cn2nc(C(=O)N3CC[C@@](C)(C(=O)O)C3)nc12] |
| 128 | 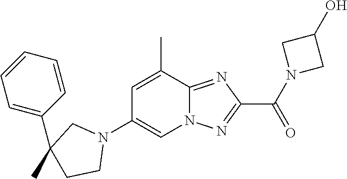 [SMILES: Cc1cc(N2CC[C@](C)(c3ccccc3)C2)cn2nc(C(=O)N3CC(O)C3)nc12] |
| 135 | 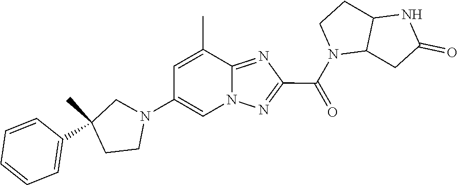 [SMILES: Cc1cc(N2CC[C@](C)(c3ccccc3)C2)cn2nc(C(=O)N3CCC4NC(=O)CC43)nc12] |
| 143 | 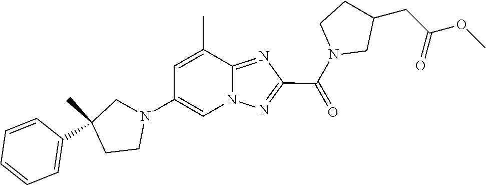 [SMILES: COC(=O)CC1CCN(C(=O)c2nc3c(C)cc(N4CC[C@](C)(c5ccccc5)C4)cn3n2)C1] |
| 144 | 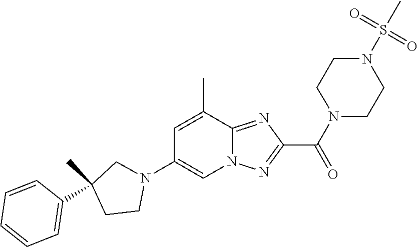 [SMILES: Cc1cc(N2CC[C@](C)(c3ccccc3)C2)cn2nc(C(=O)N3CCN(S(C)(=O)=O)CC3)nc12] |
| 146 | 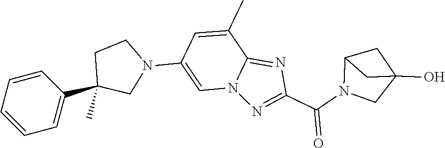 [SMILES: Cc1cc(N2CC[C@](C)(c3ccccc3)C2)cn2nc(C(=O)N3CC4(O)CC3C4)nc12] |
| 158 | 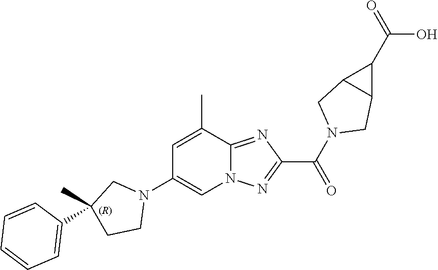 |
| 159 | 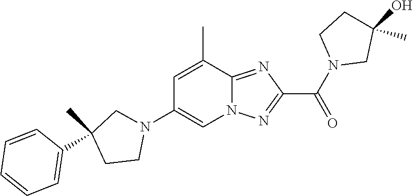 [SMILES: Cc1cc(N2CC[C@](C)(c3ccccc3)C2)cn2nc(C(=O)N3CC[C@@](C)(O)C3)nc12] |
| 174 | 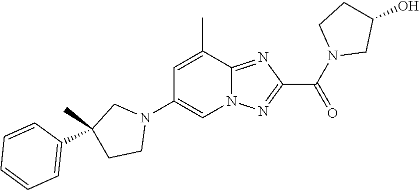 [SMILES: Cc1cc(N2CC[C@](C)(c3ccccc3)C2)cn2nc(C(=O)N3CC[C@H](O)C3)nc12] |
| 176 | 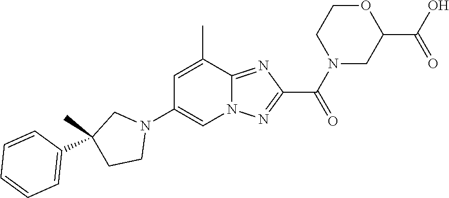 [SMILES: Cc1cc(N2CC[C@](C)(c3ccccc3)C2)cn2nc(C(=O)N3CCOC(C(=O)O)C3)nc12] |
| 177 | 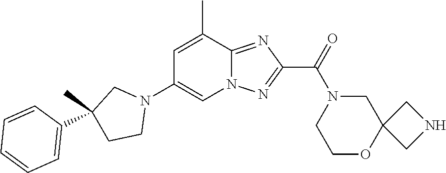 [SMILES: Cc1cc(N2CC[C@](C)(c3ccccc3)C2)cn2nc(C(=O)N3CCOC4(CNC4)C3)nc12] |
| 178 | 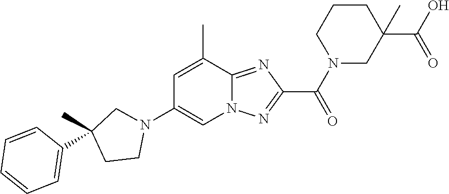 [SMILES: Cc1cc(N2CC[C@](C)(c3ccccc3)C2)cn2nc(C(=O)N3CCCC(C)(C(=O)O)C3)nc12] |
| 182 | 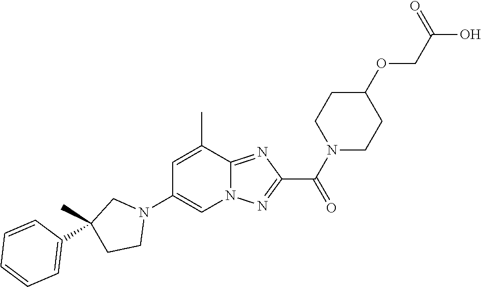 [SMILES: Cc1cc(N2CC[C@](C)(c3ccccc3)C2)cn2nc(C(=O)N3CCC(OCC(=O)O)CC3)nc12] |
| 194 | 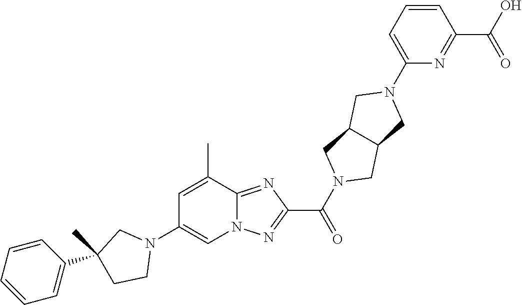 [SMILES: Cc1cc(N2CC[C@](C)(c3ccccc3)C2)cn2nc(C(=O)N3C[C@@H]4CN(c5cccc(C(=O)O)n5)C[C@@H]4C3)nc12] |
| 195 | 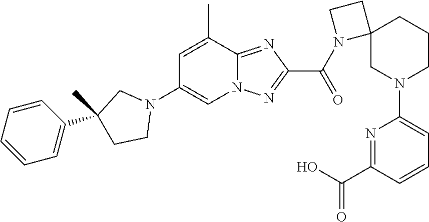 [SMILES: Cc1cc(N2CC[C@](C)(c3ccccc3)C2)cn2nc(C(=O)N3CCC34CCCN(c3cccc(C(=O)O)n3)C4)nc12] |
| 196 | 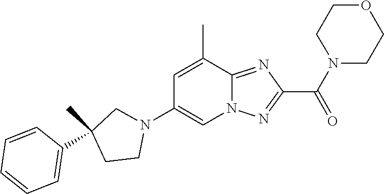 [SMILES: Cc1cc(N2CC[C@](C)(c3ccccc3)C2)cn2nc(C(=O)N3CCOCC3)nc12] |
| 197 | 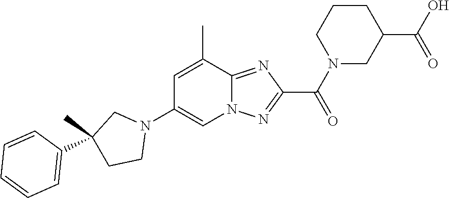 [SMILES: Cc1cc(N2CC[C@](C)(c3ccccc3)C2)cn2nc(C(=O)N3CCCC(C(=O)O)C3)nc12] |
| 210 | 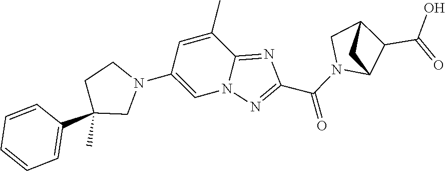 [SMILES: Cc1cc(N2CC[C@](C)(c3ccccc3)C2)cn2nc(C(=O)N3C[C@H]4C[C@@H]3C4C(=O)O)nc12] |
| 211 | 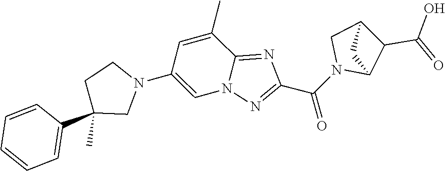 [SMILES: Cc1cc(N2CC[C@](C)(c3ccccc3)C2)cn2nc(C(=O)N3C[C@@H]4C[C@H]3C4C(=O)O)nc12] |
| 213 | 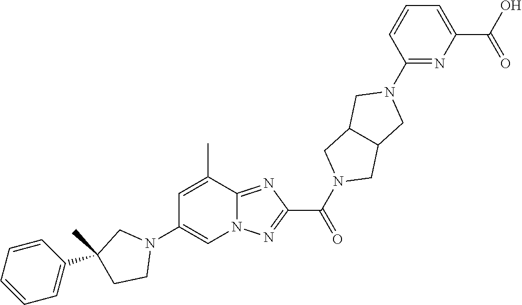 [SMILES: Cc1cc(N2CC[C@](C)(c3ccccc3)C2)cn2nc(C(=O)N3CC4CN(c5cccc(C(=O)O)n5)CC4C3)nc12] |
| 215 | 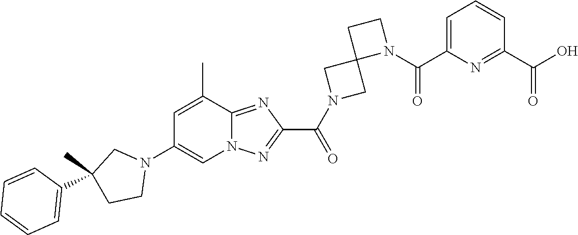 [SMILES: Cc1cc(N2CC[C@](C)(c3ccccc3)C2)cn2nc(C(=O)N3CC4(CCN4C(=O)c4cccc(C(=O)O)n4)C3)nc12] |
| 216 | 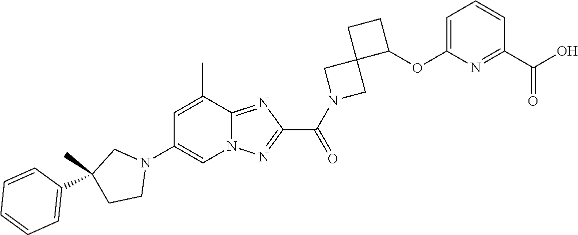 [SMILES: Cc1cc(N2CC[C@](C)(c3ccccc3)C2)cn2nc(C(=O)N3CC4(CCC4Oc4cccc(C(=O)O)n4)C3)nc12] |
| 218 | 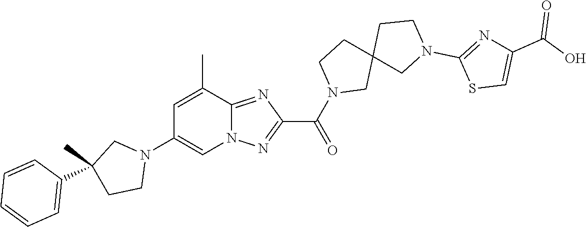 [SMILES: Cc1cc(N2CC[C@](C)(c3ccccc3)C2)cn2nc(C(=O)N3CCC4(CCN(c5nc(C(=O)O)cs5)C4)C3)nc12] |
| 221 | 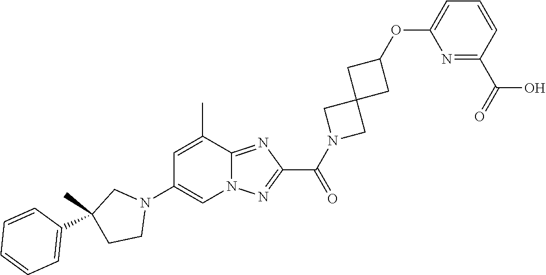 [SMILES: Cc1cc(N2CC[C@](C)(c3ccccc3)C2)cn2nc(C(=O)N3CC4(CC(Oc5cccc(C(=O)O)n5)C4)C3)nc12] |
| 222 | 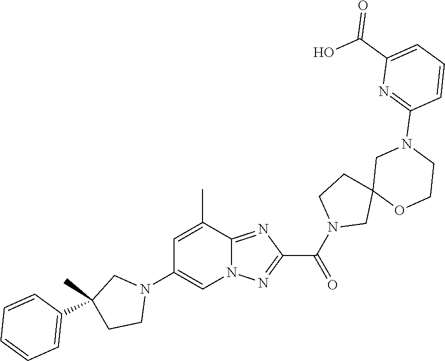 [SMILES: Cc1cc(N2CC[C@](C)(c3ccccc3)C2)cn2nc(C(=O)N3CCC4(C3)CN(c3cccc(C(=O)O)n3)CCO4)nc12] |
| 231 | 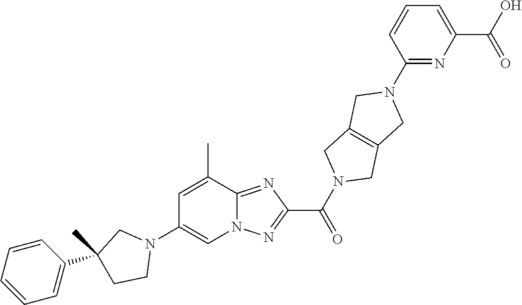 [SMILES: Cc1cc(N2CC[C@](C)(c3ccccc3)C2)cn2nc(C(=O)N3CC4=C(C3)CN(c3cccc(C(=O)O)n3)C4)nc12] |
| 233 | 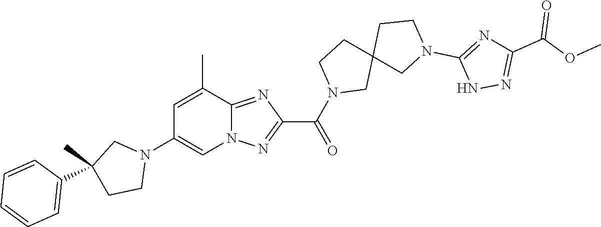 [SMILES: COC(=O)c1n[nH]c(N2CCC3(CCN(C(=O)c4nc5c(C)cc(N6CC[C@](C)(c7ccccc7)C6)cn5n4)C3)C2)n1] |
| 234 | 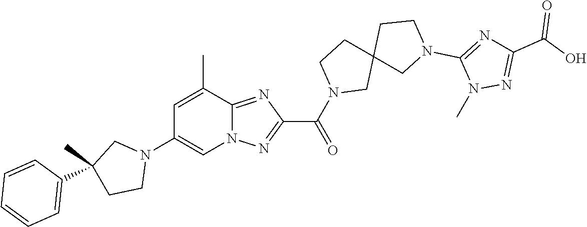 [SMILES: Cc1cc(N2CC[C@](C)(c3ccccc3)C2)cn2nc(C(=O)N3CCC4(CCN(c5nc(C(=O)O)nn5C)C4)C3)nc12] |
| 235 | 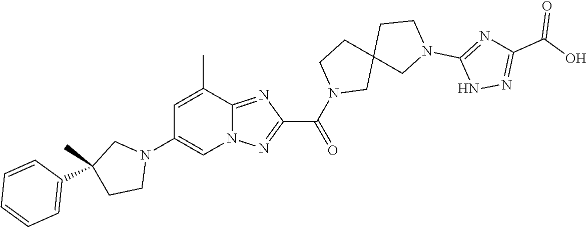 [SMILES: Cc1cc(N2CC[C@](C)(c3ccccc3)C2)cn2nc(C(=O)N3CCC4(CCN(c5nc(C(=O)O)n[nH]5)C4)C3)nc12] |
| 236 | 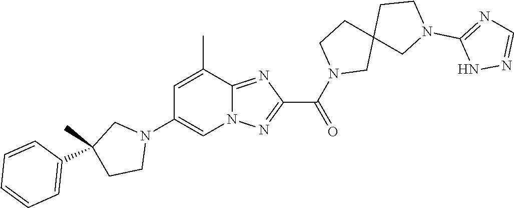 [SMILES: Cc1cc(N2CC[C@](C)(c3ccccc3)C2)cn2nc(C(=O)N3CCC4(CCN(c5ncn[nH]5)C4)C3)nc12] |
| 239 | 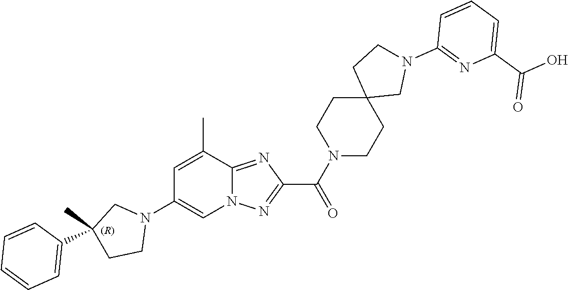 |
| 240 | 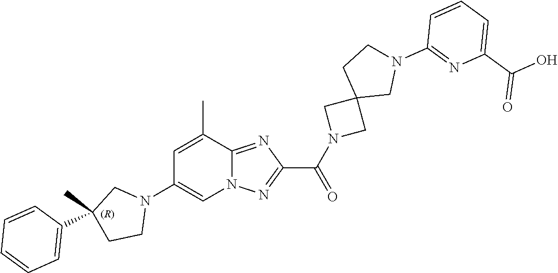 |
| 241 | 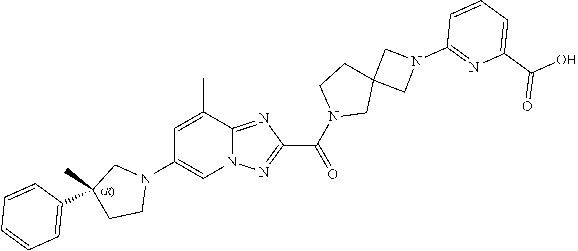 |
| 242 | 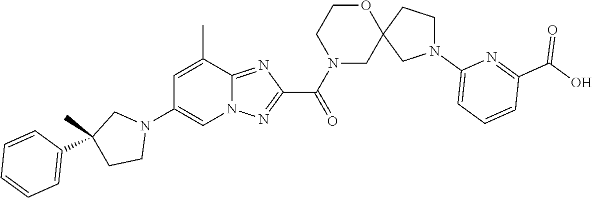 |
| 244 | 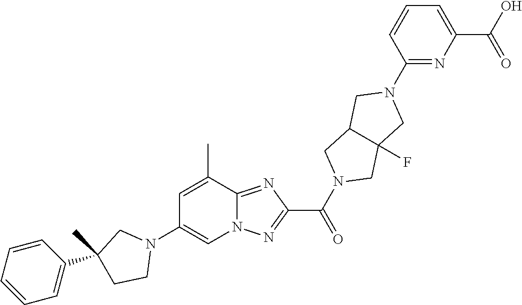 [SMILES: Cc1cc(N2CC[C@](C)(c3ccccc3)C2)cn2nc(C(=O)N3CC4CN(c5cccc(C(=O)O)n5)CC4(F)C3)nc12] |
| 246 | 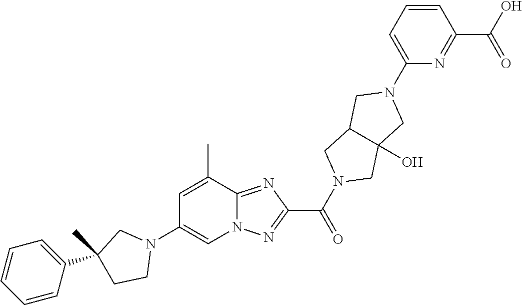 [SMILES: Cc1cc(N2CC[C@](C)(c3ccccc3)C2)cn2nc(C(=O)N3CC4CN(c5cccc(C(=O)O)n5)CC4(O)C3)nc12] |
| 247 | 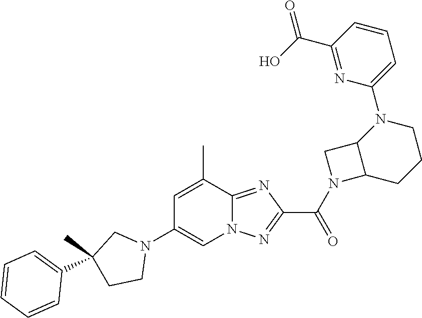 [SMILES: Cc1cc(N2CC[C@](C)(c3ccccc3)C2)cn2nc(C(=O)N3CC4C3CCCN4c3cccc(C(=O)O)n3)nc12] |
| 248 | 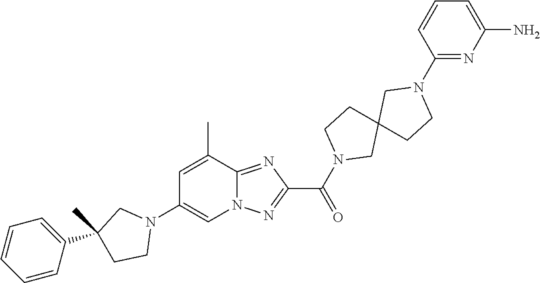 [SMILES: Cc1cc(N2CC[C@](C)(c3ccccc3)C2)cn2nc(C(=O)N3CCC4(CCN(c5cccc(N)n5)C4)C3)nc12] |
| 249 | 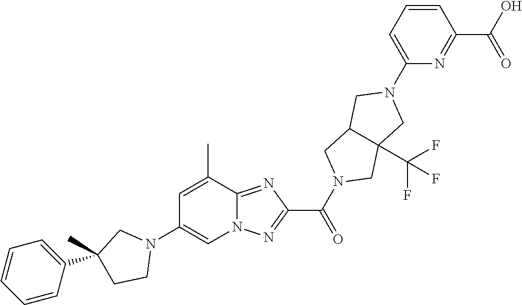 [SMILES: Cc1cc(N2CC[C@](C)(c3ccccc3)C2)cn2nc(C(=O)N3CC4CN(c5cccc(C(=O)O)n5)CC4(C(F)(F)F)C3)nc12] |
| 250 | 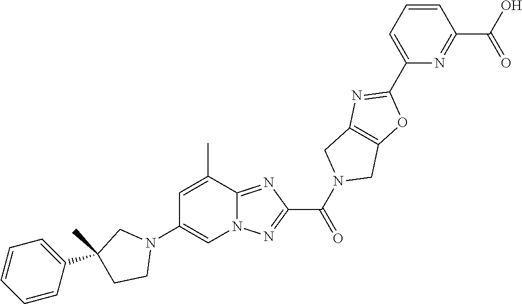 [SMILES: Cc1cc(N2CC[C@](C)(c3ccccc3)C2)cn2nc(C(=O)N3Cc4nc(-c5cccc(C(=O)O)n5)oc4C3)nc12] |
| 252 | 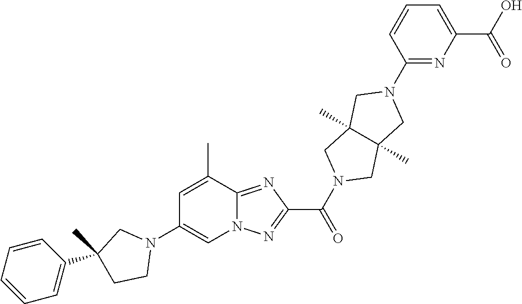 [SMILES: Cc1cc(N2CC[C@](C)(c3ccccc3)C2)cn2nc(C(=O)N3C[C@@]4(C)CN(c5cccc(C(=O)O)n5)C[C@@]4(C)C3)nc12] |
| 253 | 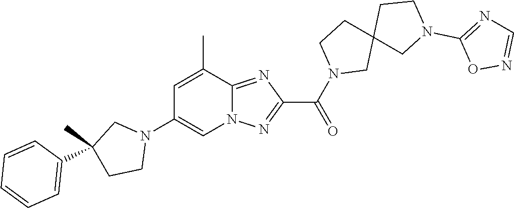 [SMILES: Cc1cc(N2CC[C@](C)(c3ccccc3)C2)cn2nc(C(=O)N3CCC4(CCN(c5ncno5)C4)C3)nc12] |
| 255 | 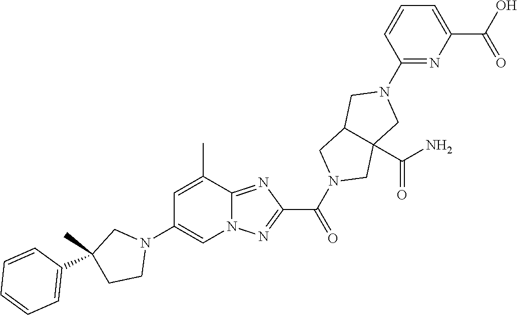 [SMILES: Cc1cc(N2CC[C@](C)(c3ccccc3)C2)cn2nc(C(=O)N3CC4CN(c5cccc(C(=O)O)n5)CC4(C(N)=O)C3)nc12] |
| 257 | 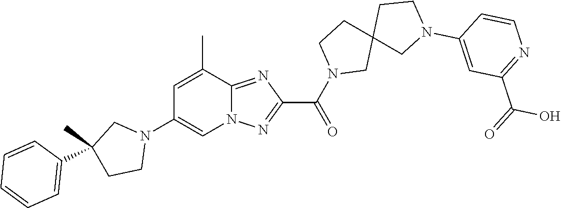 [SMILES: Cc1cc(N2CC[C@](C)(c3ccccc3)C2)cn2nc(C(=O)N3CCC4(CCN(c5ccnc(C(=O)O)c5)C4)C3)nc12] |
| 258 | 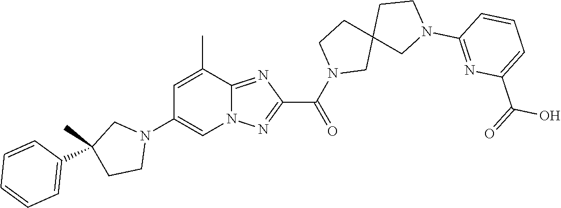 [SMILES: Cc1cc(N2CC[C@](C)(c3ccccc3)C2)cn2nc(C(=O)N3CCC4(CCN(c5cccc(C(=O)O)n5)C4)C3)nc12] |
| 259 | 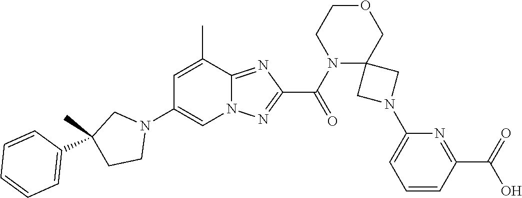 [SMILES: Cc1cc(N2CC[C@](C)(c3ccccc3)C2)cn2nc(C(=O)N3CCOCC34CN(c3cccc(C(=O)O)n3)C4)nc12] |
| 260 | 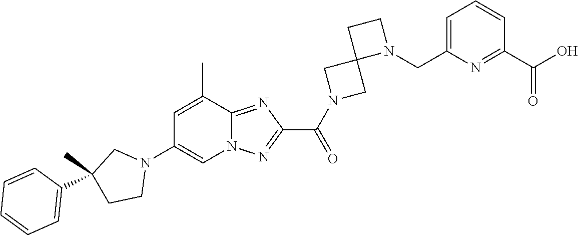 [SMILES: Cc1cc(N2CC[C@](C)(c3ccccc3)C2)cn2nc(C(=O)N3CC4(CCN4Cc4cccc(C(=O)O)n4)C3)nc12] |
| 266 | 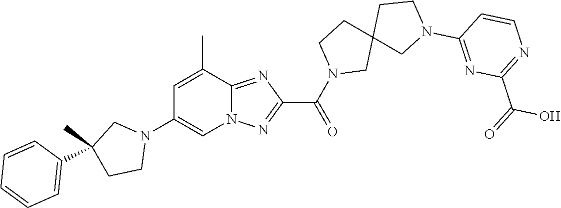 [SMILES: Cc1cc(N2CC[C@](C)(c3ccccc3)C2)cn2nc(C(=O)N3CCC4(CCN(c5ccnc(C(=O)O)n5)C4)C3)nc12] |
| 267 | 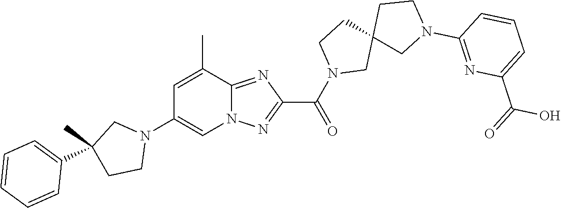 [SMILES: Cc1cc(N2CC[C@](C)(c3ccccc3)C2)cn2nc(C(=O)N3CC[C@@]4(CCN(c5cccc(C(=O)O)n5)C4)C3)nc12] |
| 268 | 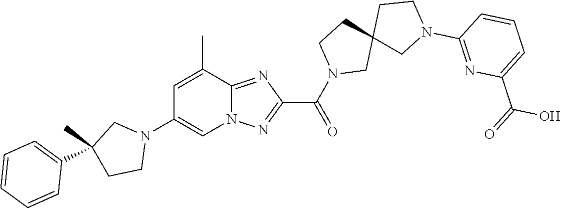 [SMILES: Cc1cc(N2CC[C@](C)(c3ccccc3)C2)cn2nc(C(=O)N3CC[C@]4(CCN(c5cccc(C(=O)O)n5)C4)C3)nc12] |
| 270 | 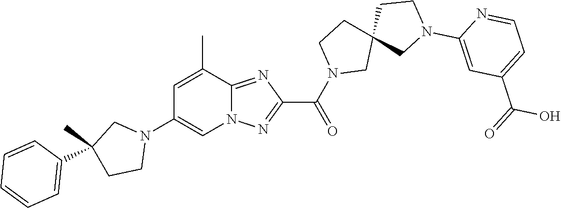 [SMILES: Cc1cc(N2CC[C@](C)(c3ccccc3)C2)cn2nc(C(=O)N3CC[C@]4(CCN(c5cc(C(=O)O)ccn5)C4)C3)nc12] |
| 275 | 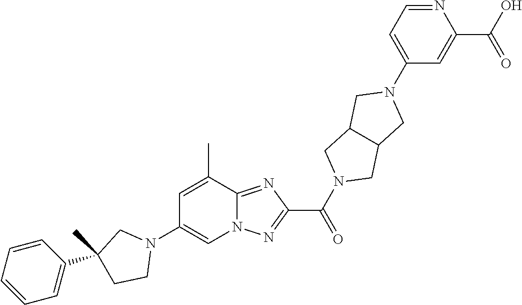 [SMILES: Cc1cc(N2CC[C@](C)(c3ccccc3)C2)cn2nc(C(=O)N3CC4CN(c5ccnc(C(=O)O)c5)CC4C3)nc12] |
| 278 | 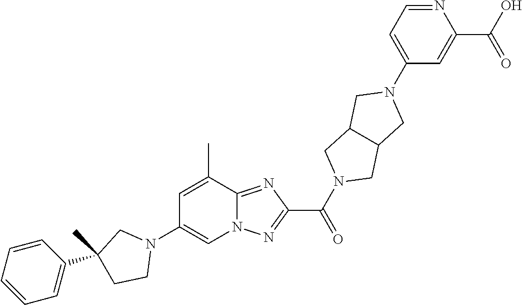 [SMILES: Cc1cc(N2CC[C@](C)(c3ccccc3)C2)cn2nc(C(=O)N3CC4CN(c5ccnc(C(=O)O)c5)CC4C3)nc12] |
| 279 | 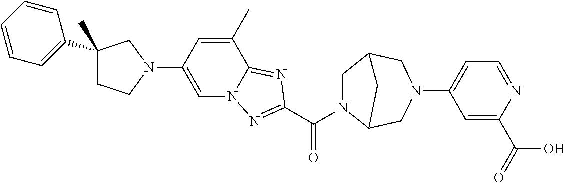 [SMILES: Cc1cc(N2CC[C@](C)(c3ccccc3)C2)cn2nc(C(=O)N3CC4CC3CN(c3ccnc(C(=O)O)c3)C4)nc12] |
| 280 | 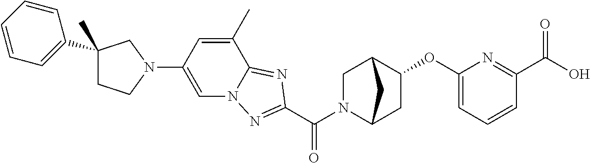 [SMILES: Cc1cc(N2CC[C@](C)(c3ccccc3)C2)cn2nc(C(=O)N3C[C@H]4C[C@@H]3C[C@H]4Oc3cccc(C(=O)O)n3)nc12] |
| 281 | 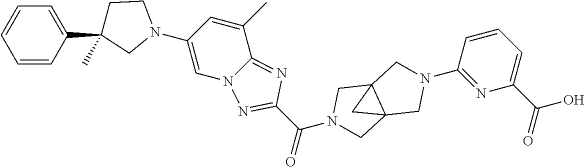 [SMILES: Cc1cc(N2CC[C@](C)(c3ccccc3)C2)cn2nc(C(=O)N3CC45CN(c6cccc(C(=O)O)n6)CC4(C3)C5)nc12] |
| 286 | 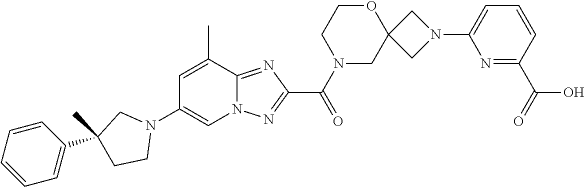 [SMILES: Cc1cc(N2CC[C@](C)(c3ccccc3)C2)cn2nc(C(=O)N3CCOC4(C3)CN(c3cccc(C(=O)O)n3)C4)nc12] |
| 287 | 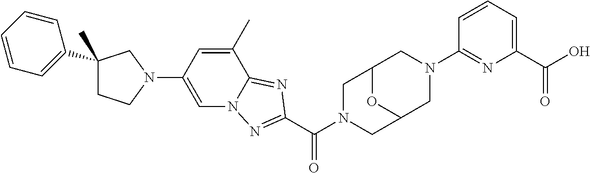 [SMILES: Cc1cc(N2CC[C@](C)(c3ccccc3)C2)cn2nc(C(=O)N3CC4CN(c5cccc(C(=O)O)n5)CC(C3)O4)nc12] |
| 288 | 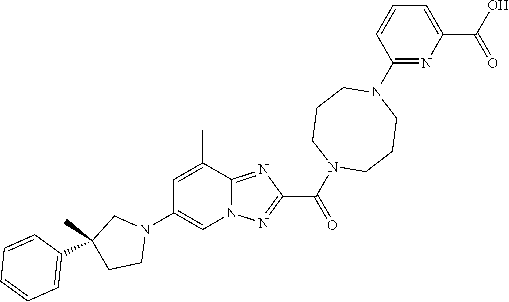 [SMILES: Cc1cc(N2CC[C@](C)(c3ccccc3)C2)cn2nc(C(=O)N3CCCN(c4cccc(C(=O)O)n4)CCC3)nc12] |
| 289 | 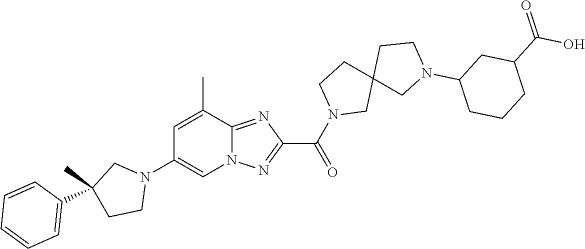 [SMILES: Cc1cc(N2CC[C@](C)(c3ccccc3)C2)cn2nc(C(=O)N3CCC4(CCN(C5CCCC(C(=O)O)C5)C4)C3)nc12] |
| 21 | 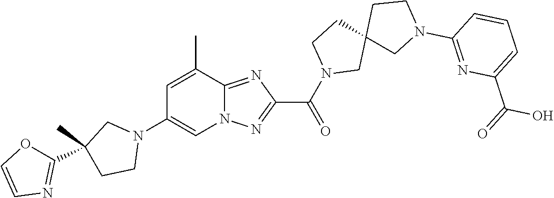 [SMILES: Cc1cc(N2CC[C@](C)(c3ncco3)C2)cn2nc(C(=O)N3CC[C@@]4(CCN(c5cccc(C(=O)O)n5)C4)C3)nc12] |
| 293 | 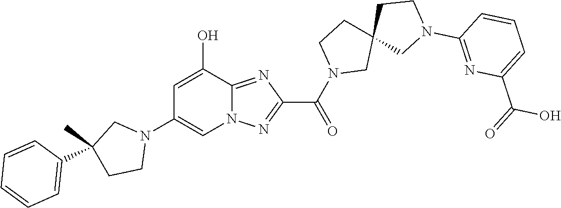 [SMILES: C[C@]1(c2ccccc2)CCN(c2cc(O)c3nc(C(=O)N4CC[C@@]5(CCN(c6cccc(C(=O)O)n6)C5)C4)nn3c2)C1] |
| 294 | 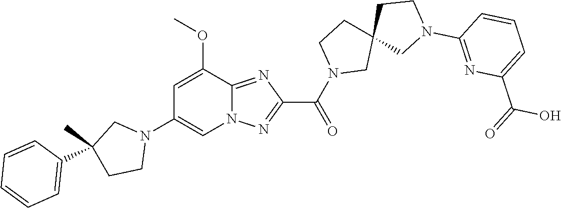 [SMILES: COc1cc(N2CC[C@](C)(c3ccccc3)C2)cn2nc(C(=O)N3CC[C@@]4(CCN(c5cccc(C(=O)O)n5)C4)C3)nc12] |
| 23 | 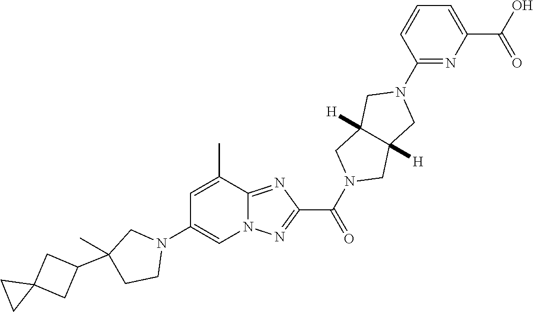 [SMILES: Cc1cc(N2CCC(C)(C3CC4(CC4)C3)C2)cn2nc(C(=O)N3C[C@@H]4CN(c5cccc(C(=O)O)n5)C[C@@H]4C3)nc12] |
| 296 | 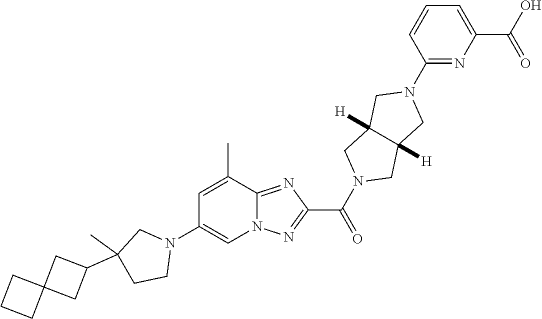 [SMILES: Cc1cc(N2CCC(C)(C3CC4(CCC4)C3)C2)cn2nc(C(=O)N3C[C@@H]4CN(c5cccc(C(=O)O)n5)C[C@@H]4C3)nc12] |
| 298 | 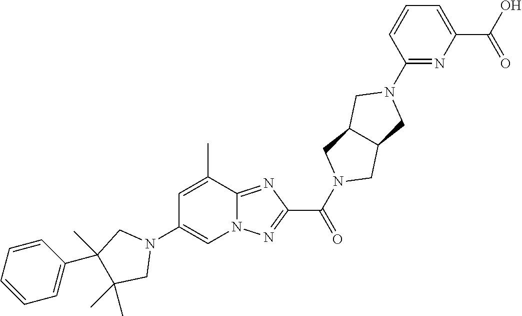 [SMILES: Cc1cc(N2CC(C)(C)C(C)(c3ccccc3)C2)cn2nc(C(=O)N3C[C@@H]4CN(c5cccc(C(=O)O)n5)C[C@@H]4C3)nc12] |
| 299 | 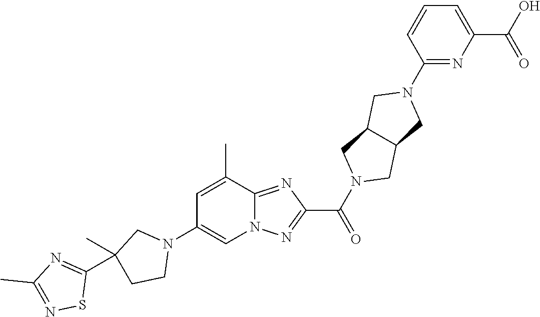 [SMILES: Cc1nsc(C2(C)CCN(c3cc(C)c4nc(C(=O)N5C[C@@H]6CN(c7cccc(C(=O)O)n7)C[C@@H]6C5)nn4c3)C2)n1] |
| 300 | 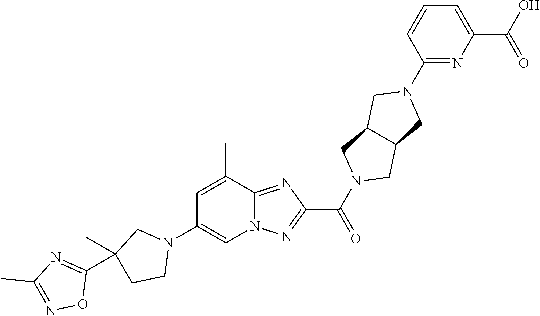 [SMILES: Cc1noc(C2(C)CCN(c3cc(C)c4nc(C(=O)N5C[C@@H]6CN(c7cccc(C(=O)O)n7)C[C@@H]6C5)nn4c3)C2)n1] |
| 301 | 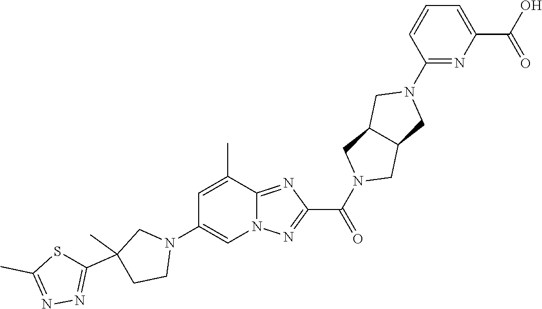 [SMILES: Cc1nnc(C2(C)CCN(c3cc(C)c4nc(C(=O)N5C[C@@H]6CN(c7cccc(C(=O)O)n7)C[C@@H]6C5)nn4c3)C2)s1] |
| 302 | 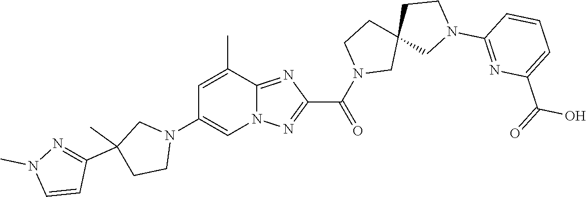 [SMILES: Cc1cc(N2CCC(C)(c3ccn(C)n3)C2)cn2nc(C(=O)N3CC[C@@]4(CCN(c5cccc(C(=O)O)n5)C4)C3)nc12] |
| 331 | 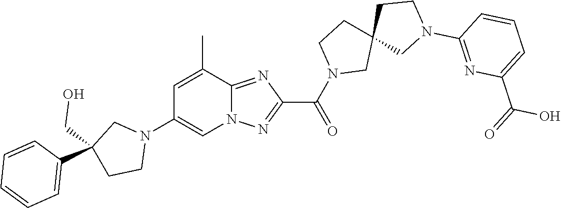 [SMILES: Cc1cc(N2CC[C@@](CO)(c3ccccc3)C2)cn2nc(C(=O)N3CC[C@@]4(CCN(c5cccc(C(=O)O)n5)C4)C3)nc12] |
| 332 | 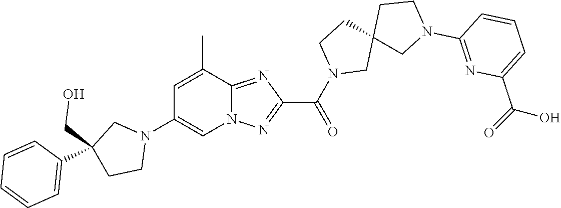 [SMILES: Cc1cc(N2CC[C@](CO)(c3ccccc3)C2)cn2nc(C(=O)N3CC[C@@]4(CCN(c5cccc(C(=O)O)n5)C4)C3)nc12] |
| 334 | 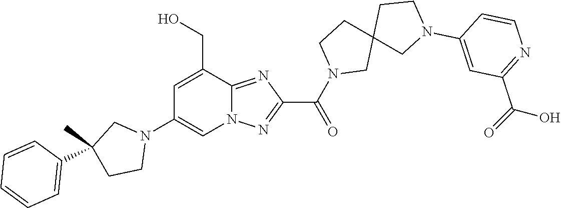 [SMILES: C[C@]1(c2ccccc2)CCN(c2cc(CO)c3nc(C(=O)N4CCC5(CCN(c6ccnc(C(=O)O)c6)C5)C4)nn3c2)C1] |
| 343 | 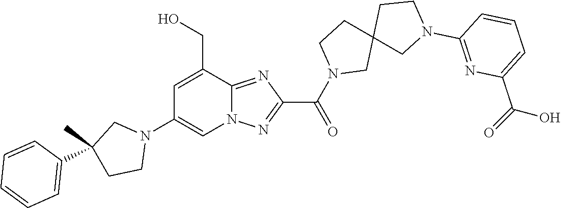 [SMILES: C[C@]1(c2ccccc2)CCN(c2cc(CO)c3nc(C(=O)N4CCC5(CCN(c6cccc(C(=O)O)n6)C5)C4)nn3c2)C1] |
| 345 | 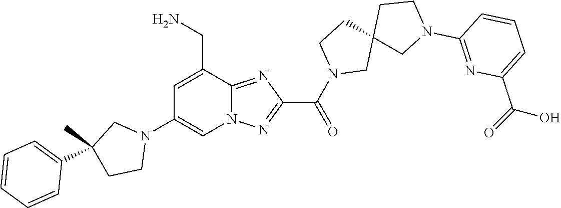 [SMILES: C[C@]1(c2ccccc2)CCN(c2cc(CN)c3nc(C(=O)N4CC[C@@]5(CCN(c6cccc(C(=O)O)n6)C5)C4)nn3c2)C1] |
| 347 | 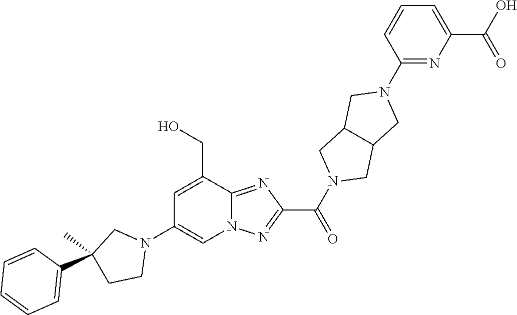 [SMILES: C[C@@]1(c2ccccc2)CCN(c2cc(CO)c3nc(C(=O)N4CC5CN(c6cccc(C(=O)O)n6)CC5C4)nn3c2)C1] |
| 353 | 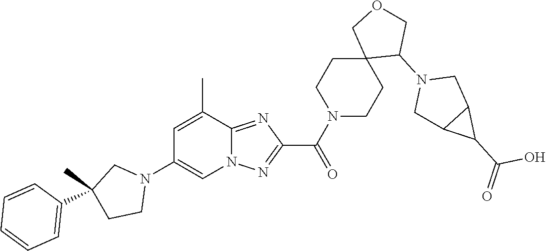 [SMILES: Cc1cc(N2CC[C@](C)(c3ccccc3)C2)cn2nc(C(=O)N3CCC4(CC3)COCC4N3CC4C(C3)C4C(=O)O)nc12] |
| 355 | 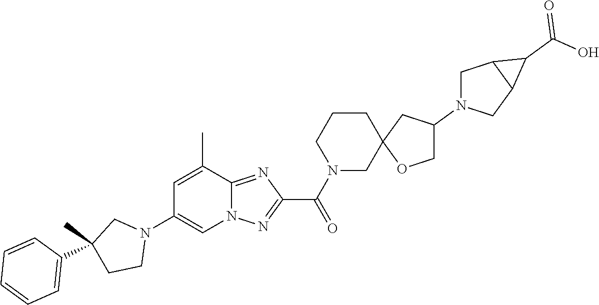 [SMILES: Cc1cc(N2CC[C@](C)(c3ccccc3)C2)cn2nc(C(=O)N3CCCC4(CC(N5CC6C(C5)C6C(=O)O)CO4)C3)nc12] |
| 366 | 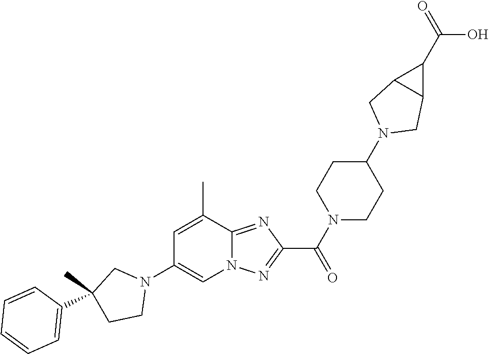 [SMILES: Cc1cc(N2CC[C@](C)(c3ccccc3)C2)cn2nc(C(=O)N3CCC(N4CC5C(C4)C5C(=O)O)CC3)nc12] |
| 370 | 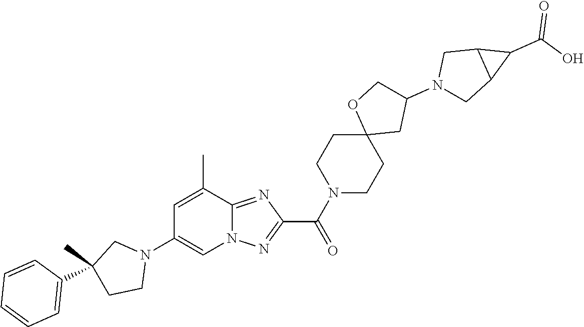 [SMILES: Cc1cc(N2CC[C@](C)(c3ccccc3)C2)cn2nc(C(=O)N3CCC4(CC3)CC(N3CC5C(C3)C5C(=O)O)CO4)nc12] |
| 375 | 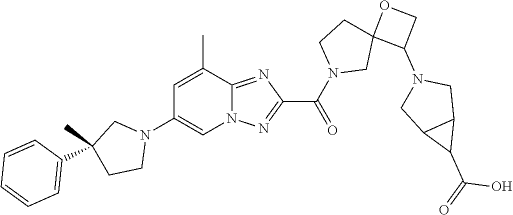 [SMILES: Cc1cc(N2CC[C@](C)(c3ccccc3)C2)cn2nc(C(=O)N3CCC4(C3)OCC4N3CC4C(C3)C4C(=O)O)nc12] |
| 376 | 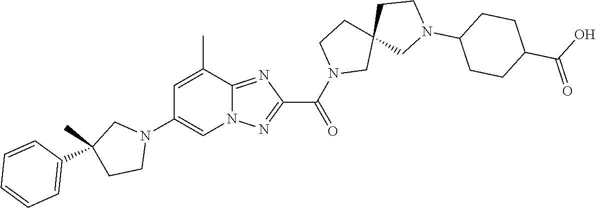 [SMILES: Cc1cc(N2CC[C@](C)(c3ccccc3)C2)cn2nc(C(=O)N3CC[C@@]4(CCN(C5CCC(C(=O)O)CC5)C4)C3)nc12] |
| 377 | 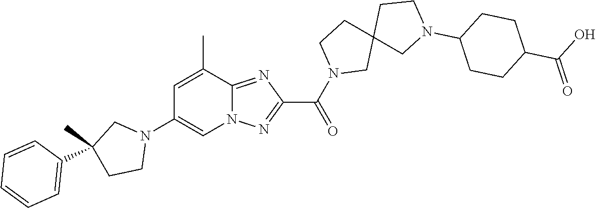 [SMILES: Cc1cc(N2CC[C@](C)(c3ccccc3)C2)cn2nc(C(=O)N3CCC4(CCN(C5CCC(C(=O)O)CC5)C4)C3)nc12] |
| 385 | 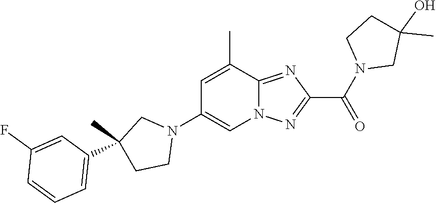 [SMILES: Cc1cc(N2CC[C@](C)(c3cccc(F)c3)C2)cn2nc(C(=O)N3CCC(C)(O)C3)nc12] |
| 388 | 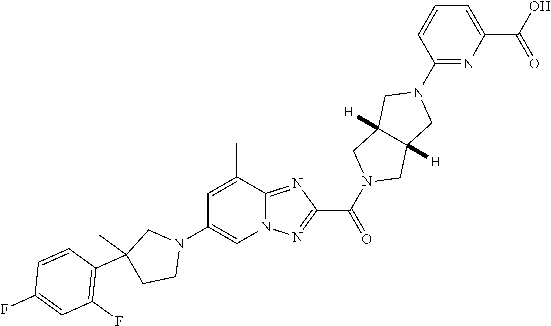 [SMILES: Cc1cc(N2CCC(C)(c3ccc(F)cc3F)C2)cn2nc(C(=O)N3C[C@@H]4CN(c5cccc(C(=O)O)n5)C[C@@H]4C3)nc12] |
| 389 | 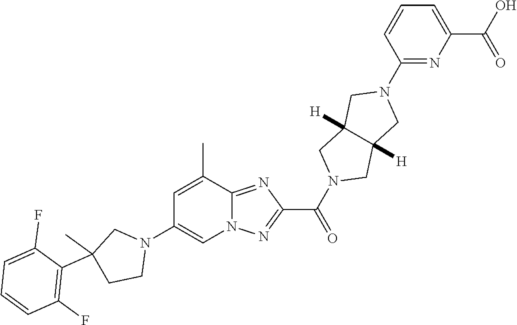 [SMILES: Cc1cc(N2CCC(C)(c3c(F)cccc3F)C2)cn2nc(C(=O)N3C[C@@H]4CN(c5cccc(C(=O)O)n5)C[C@@H]4C3)nc12] |
| 393 | 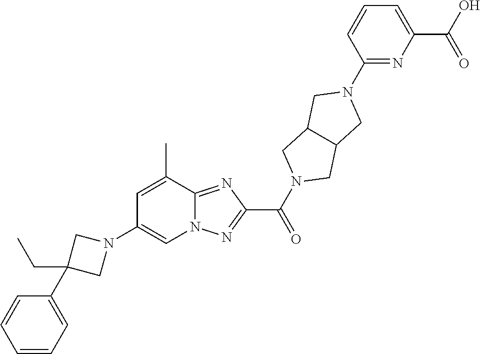 [SMILES: CCC1(c2ccccc2)CN(c2cc(C)c3nc(C(=O)N4CC5CN(c6cccc(C(=O)O)n6)CC5C4)nn3c2)C1] |
| 394 | 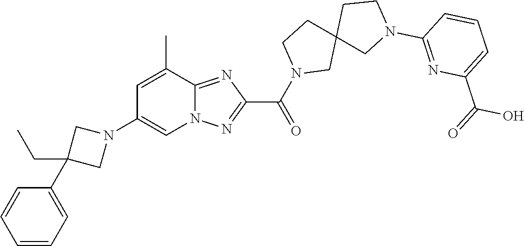 [SMILES: CCC1(c2ccccc2)CN(c2cc(C)c3nc(C(=O)N4CCC5(CCN(c6cccc(C(=O)O)n6)C5)C4)nn3c2)C1] |
| 396 | 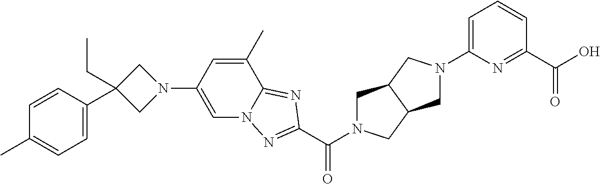 [SMILES: CCC1(c2ccc(C)cc2)CN(c2cc(C)c3nc(C(=O)N4C[C@@H]5CN(c6cccc(C(=O)O)n6)C[C@@H]5C4)nn3c2)C1] |
| 397 | 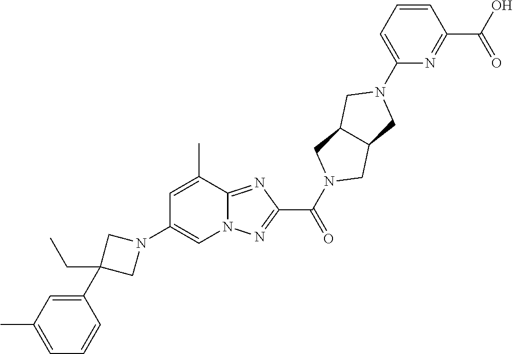 [SMILES: CCC1(c2cccc(C)c2)CN(c2cc(C)c3nc(C(=O)N4C[C@@H]5CN(c6cccc(C(=O)O)n6)C[C@@H]5C4)nn3c2)C1] |
| 398 | 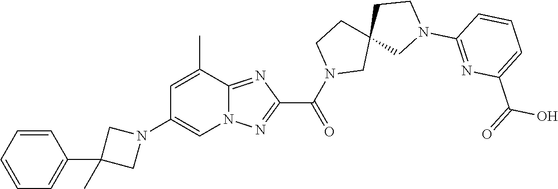 [SMILES: Cc1cc(N2CC(C)(c3ccccc3)C2)cn2nc(C(=O)N3CC[C@@]4(CCN(c5cccc(C(=O)O)n5)C4)C3)nc12] |
| 399 | 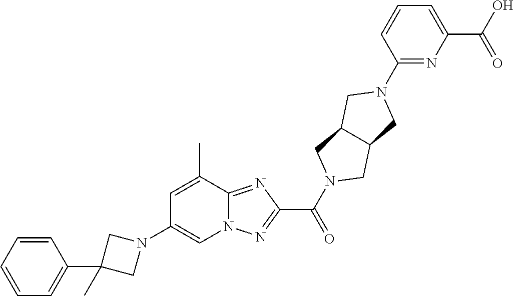 [SMILES: Cc1cc(N2CC(C)(c3ccccc3)C2)cn2nc(C(=O)N3C[C@@H]4CN(c5cccc(C(=O)O)n5)C[C@@H]4C3)nc12] |
| 400 | 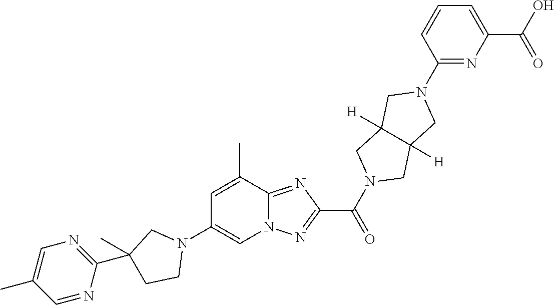 [SMILES: Cc1cnc(C2(C)CCN(c3cc(C)c4nc(C(=O)N5CC6CN(c7cccc(C(=O)O)n7)CC6C5)nn4c3)C2)nc1] |
| 35 | 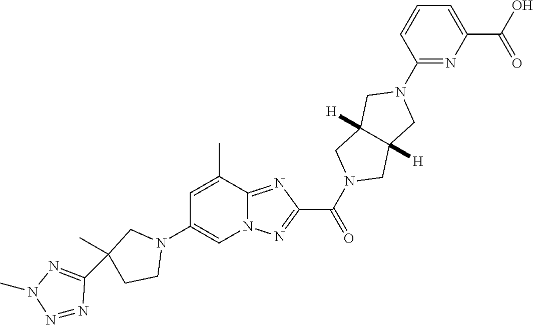 [SMILES: Cc1cc(N2CCC(C)(c3nnn(C)n3)C2)cn2nc(C(=O)N3C[C@@H]4CN(c5cccc(C(=O)O)n5)C[C@@H]4C3)nc12] |
| 401 | 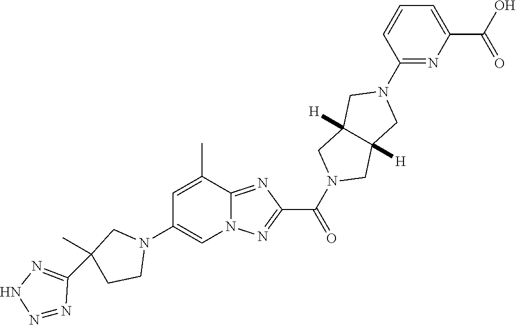 [SMILES: Cc1cc(N2CCC(C)(c3nn[nH]n3)C2)cn2nc(C(=O)N3C[C@@H]4CN(c5cccc(C(=O)O)n5)C[C@@H]4C3)nc12] |
| 36 | 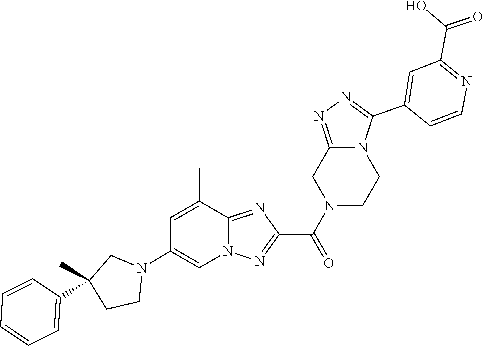 [SMILES: Cc1cc(N2CC[C@](C)(c3ccccc3)C2)cn2nc(C(=O)N3CCn4c(nnc4-c4ccnc(C(=O)O)c4)C3)nc12] |
| 402 | 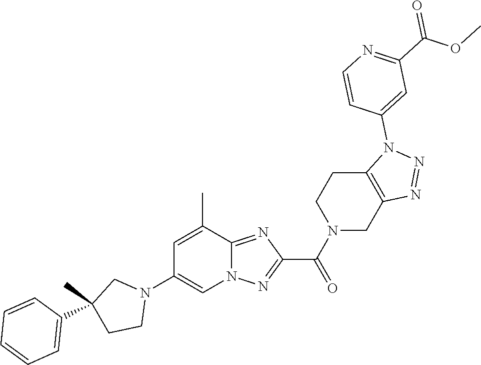 [SMILES: COC(=O)c1cc(-n2nnc3c2CCN(C(=O)c2nc4c(C)cc(N5CC[C@](C)(c6ccccc6)C5)cn4n2)C3)ccn1] |
| 403 | 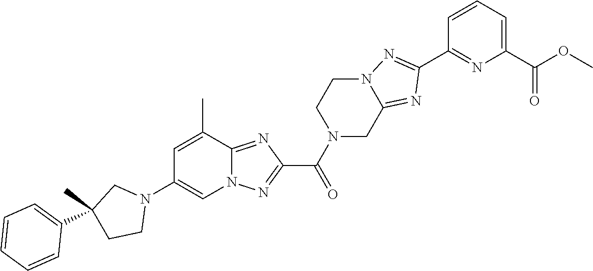 [SMILES: COC(=O)c1cccc(-c2nc3n(n2)CCN(C(=O)c2nc4c(C)cc(N5CC[C@](C)(c6ccccc6)C5)cn4n2)C3)n1] |
| 404 | 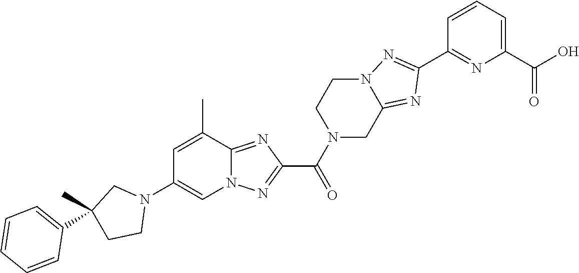 [SMILES: Cc1cc(N2CC[C@](C)(c3ccccc3)C2)cn2nc(C(=O)N3CCn4nc(-c5cccc(C(=O)O)n5)nc4C3)nc12] |
| 405 | 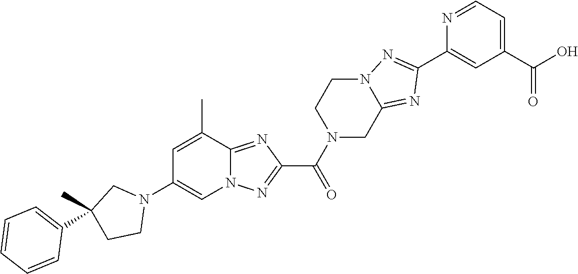 [SMILES: Cc1cc(N2CC[C@](C)(c3ccccc3)C2)cn2nc(C(=O)N3CCn4nc(-c5cc(C(=O)O)ccn5)nc4C3)nc12] |
| 406 | 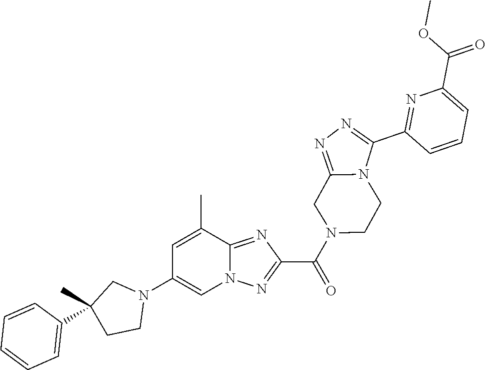 [SMILES: COC(=O)c1cccc(-c2nnc3n2CCN(C(=O)c2nc4c(C)cc(N5CC[C@](C)(c6ccccc6)C5)cn4n2)C3)n1] |
| 407 | 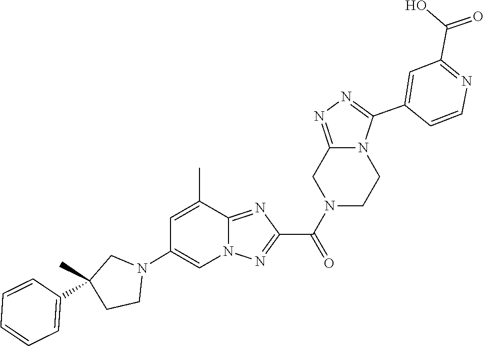 [SMILES: Cc1cc(N2CC[C@](C)(c3ccccc3)C2)cn2nc(C(=O)N3CCn4c(nnc4-c4ccnc(C(=O)O)c4)C3)nc12] |
| 408 | 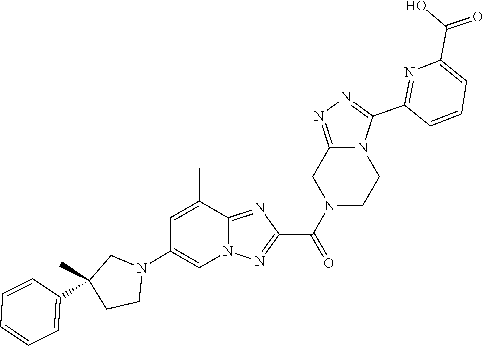 [SMILES: Cc1cc(N2CC[C@](C)(c3ccccc3)C2)cn2nc(C(=O)N3CCn4c(nnc4-c4cccc(C(=O)O)n4)C3)nc12] |
| 37 | 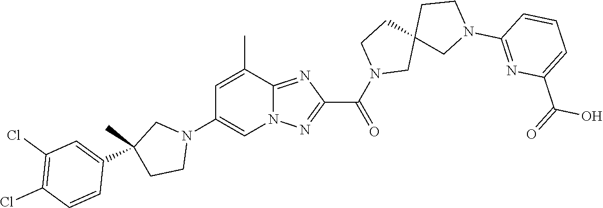 [SMILES: Cc1cc(N2CC[C@](C)(c3ccc(Cl)c(Cl)c3)C2)cn2nc(C(=O)N3CC[C@@]4(CCN(c5cccc(C(=O)O)n5)C4)C3)nc12] |
| 409 | 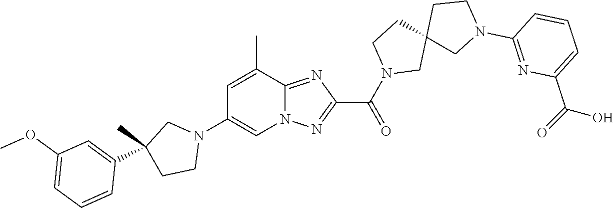 [SMILES: COc1cccc([C@@]2(C)CCN(c3cc(C)c4nc(C(=O)N5CC[C@@]6(CCN(c7cccc(C(=O)O)n7)C6)C5)nn4c3)C2)c1] |
| 410 | 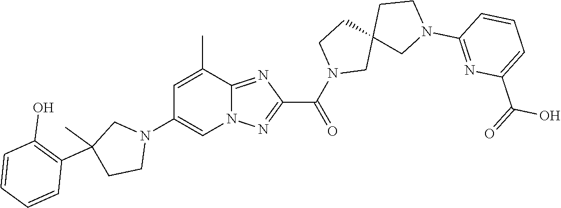 [SMILES: Cc1cc(N2CCC(C)(c3ccccc3O)C2)cn2nc(C(=O)N3CC[C@@]4(CCN(c5cccc(C(=O)O)n5)C4)C3)nc12] |
| 411 | 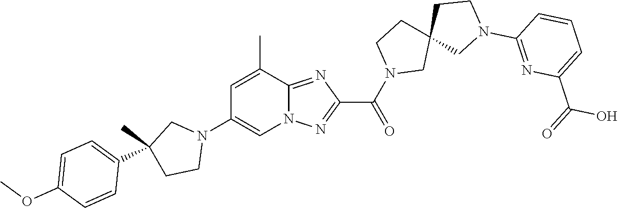 [SMILES: COc1ccc([C@@]2(C)CCN(c3cc(C)c4nc(C(=O)N5CC[C@@]6(CCN(c7cccc(C(=O)O)n7)C6)C5)nn4c3)C2)cc1] |
| 762 | 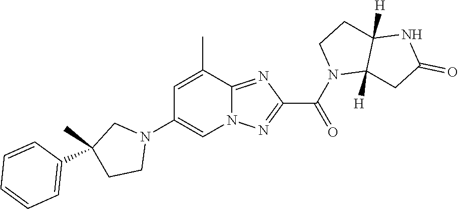 [SMILES: Cc1cc(N2CC[C@](C)(c3ccccc3)C2)cn2nc(C(=O)N3CC[C@@H]4NC(=O)C[C@@H]43)nc12] |
| 763 | 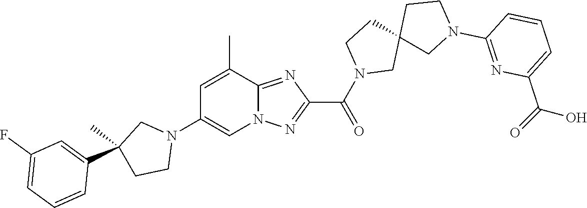 [SMILES: Cc1cc(N2CC[C@@](C)(c3cccc(F)c3)C2)cn2nc(C(=O)N3CC[C@@]4(CCN(c5cccc(C(=O)O)n5)C4)C3)nc12] |
| 413 | 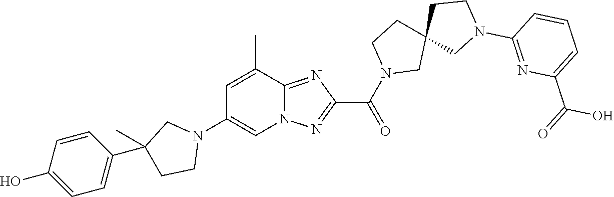 [SMILES: Cc1cc(N2CCC(C)(c3ccc(O)cc3)C2)cn2nc(C(=O)N3CC[C@@]4(CCN(c5cccc(C(=O)O)n5)C4)C3)nc12] |
| 414 | 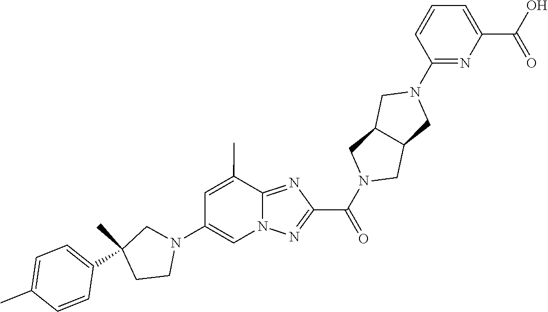 [SMILES: Cc1ccc([C@@]2(C)CCN(c3cc(C)c4nc(C(=O)N5C[C@@H]6CN(c7cccc(C(=O)O)n7)C[C@@H]6C5)nn4c3)C2)cc1] |
| 415 | 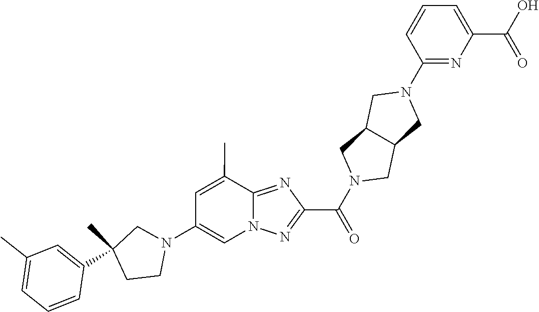 [SMILES: Cc1cccc([C@@]2(C)CCN(c3cc(C)c4nc(C(=O)N5C[C@@H]6CN(c7cccc(C(=O)O)n7)C[C@@H]6C5)nn4c3)C2)c1] |
| 416 | 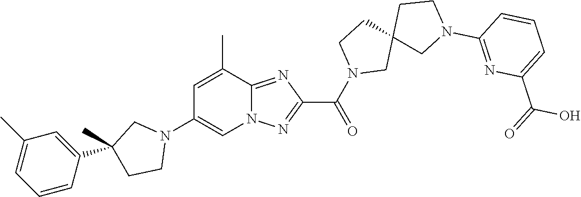 [SMILES: Cc1cccc([C@@]2(C)CCN(c3cc(C)c4nc(C(=O)N5CC[C@@]6(CCN(c7cccc(C(=O)O)n7)C6)C5)nn4c3)C2)c1] |
| 417 | 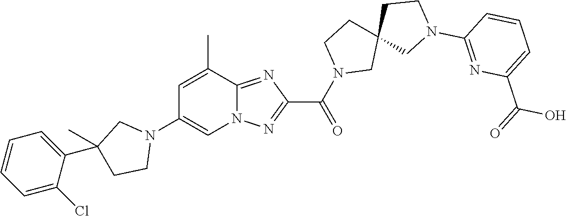 [SMILES: Cc1cc(N2CCC(C)(c3ccccc3Cl)C2)cn2nc(C(=O)N3CC[C@@]4(CCN(c5cccc(C(=O)O)n5)C4)C3)nc12] |
| 418 | 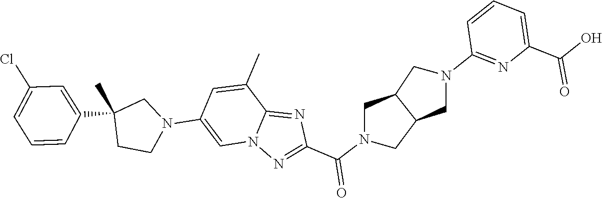 [SMILES: Cc1cc(N2CC[C@](C)(c3cccc(Cl)c3)C2)cn2nc(C(=O)N3C[C@@H]4CN(c5cccc(C(=O)O)n5)C[C@@H]4C3)nc12] |
| 419 | 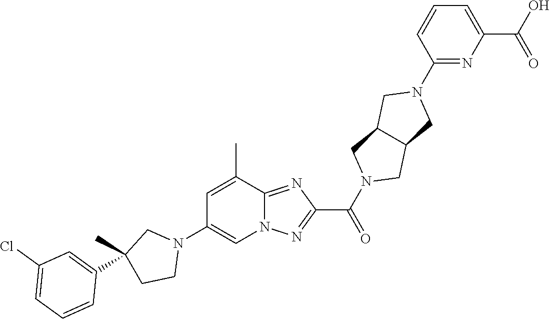 [SMILES: Cc1cc(N2CC[C@](C)(c3cccc(Cl)c3)C2)cn2nc(C(=O)N3C[C@@H]4CN(c5cccc(C(=O)O)n5)C[C@@H]4C3)nc12] |
| 420 | 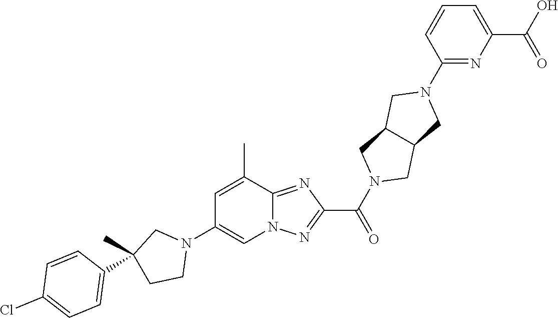 [SMILES: Cc1cc(N2CC[C@](C)(c3ccc(Cl)cc3)C2)cn2nc(C(=O)N3C[C@@H]4CN(c5cccc(C(=O)O)n5)C[C@@H]4C3)nc12] |
| 421 | 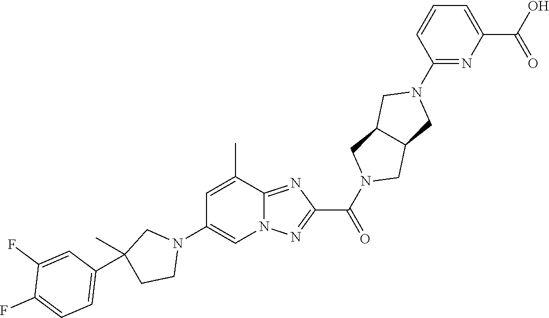 [SMILES: Cc1cc(N2CCC(C)(c3ccc(F)c(F)c3)C2)cn2nc(C(=O)N3C[C@@H]4CN(c5cccc(C(=O)O)n5)C[C@@H]4C3)nc12] |
| 423 | 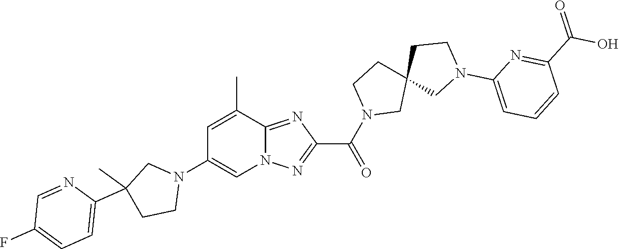 |
| 424 | 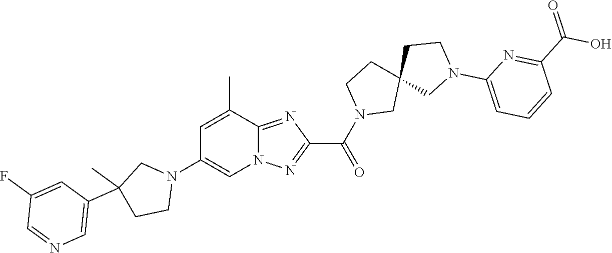 |
| 426 | 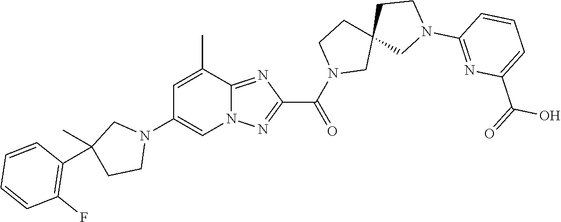 [SMILES: Cc1cc(N2CCC(C)(c3ccccc3F)C2)cn2nc(C(=O)N3CC[C@@]4(CCN(c5cccc(C(=O)O)n5)C4)C3)nc12] |
| 427 | 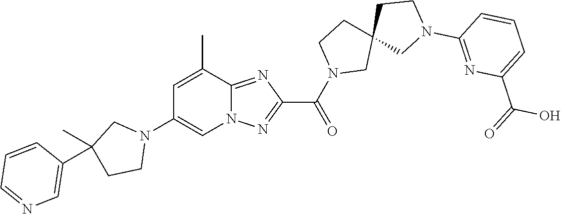 [SMILES: Cc1cc(N2CCC(C)(c3cccnc3)C2)cn2nc(C(=O)N3CC[C@@]4(CCN(c5cccc(C(=O)O)n5)C4)C3)nc12] |
| 428 | 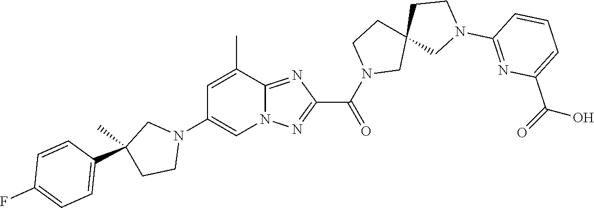 [SMILES: Cc1cc(N2CC[C@@](C)(c3ccc(F)cc3)C2)cn2nc(C(=O)N3CC[C@@]4(CCN(c5cccc(C(=O)O)n5)C4)C3)nc12] |
| 429 | 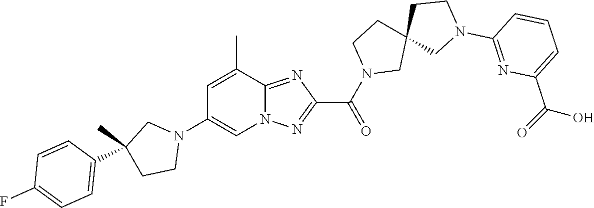 [SMILES: Cc1cc(N2CC[C@](C)(c3ccc(F)cc3)C2)cn2nc(C(=O)N3CC[C@@]4(CCN(c5cccc(C(=O)O)n5)C4)C3)nc12] |
| 431 | 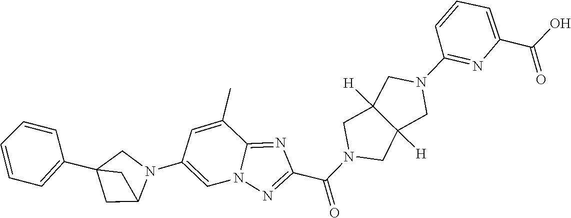 [SMILES: Cc1cc(N2CC3(c4ccccc4)CC2C3)cn2nc(C(=O)N3CC4CN(c5cccc(C(=O)O)n5)CC4C3)nc12] |
| 432 | 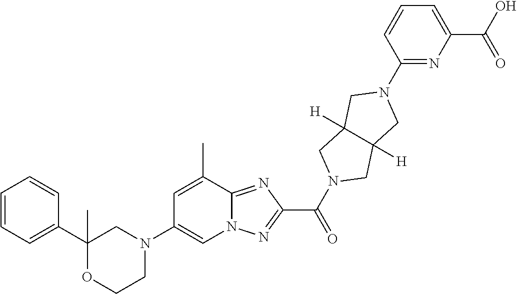 [SMILES: Cc1cc(N2CCOC(C)(c3ccccc3)C2)cn2nc(C(=O)N3CC4CN(c5cccc(C(=O)O)n5)CC4C3)nc12] |
| 439 | 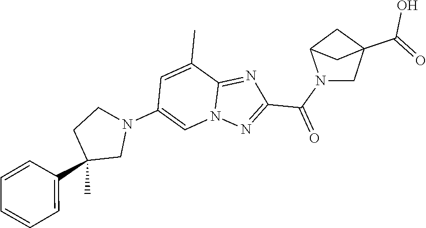 [SMILES: Cc1cc(N2CC[C@](C)(c3ccccc3)C2)cn2nc(C(=O)N3CC4(C(=O)O)CC3C4)nc12] |
| 592 | 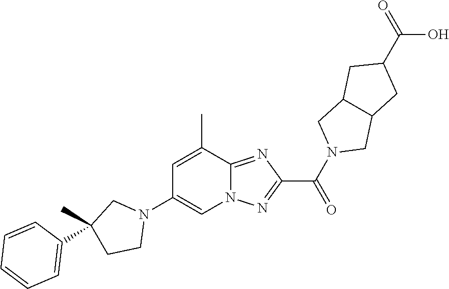 [SMILES: Cc1cc(N2CC[C@](C)(c3ccccc3)C2)cn2nc(C(=O)N3CC4CC(C(=O)O)CC4C3)nc12] |
| 594 | 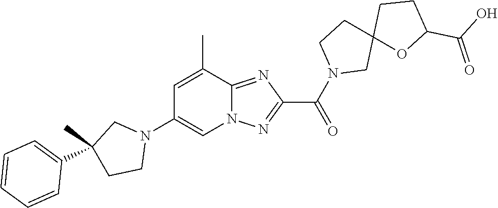 [SMILES: Cc1cc(N2CC[C@](C)(c3ccccc3)C2)cn2nc(C(=O)N3CCC4(CCC(C(=O)O)O4)C3)nc12] |
| 595 | 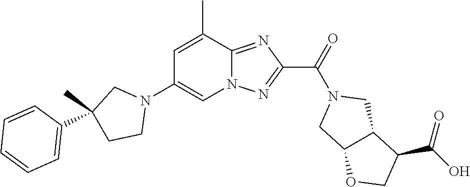 [SMILES: Cc1cc(N2CC[C@](C)(c3ccccc3)C2)cn2nc(C(=O)N3C[C@@H]4[C@@H](C(=O)O)CO[C@@H]4C3)nc12] |
| 598 | 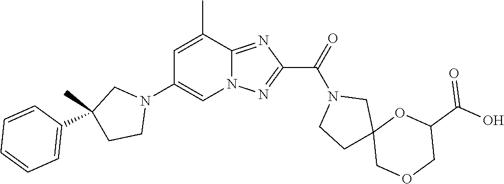 [SMILES: Cc1cc(N2CC[C@](C)(c3ccccc3)C2)cn2nc(C(=O)N3CCC4(COCC(C(=O)O)O4)C3)nc12] |
| 603 | 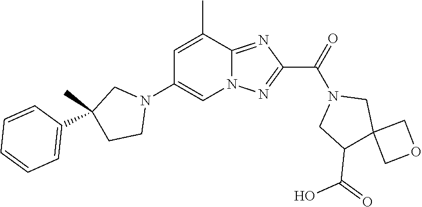 [SMILES: Cc1cc(N2CC[C@](C)(c3ccccc3)C2)cn2nc(C(=O)N3CC(C(=O)O)C4(COC4)C3)nc12] |
| 604 | 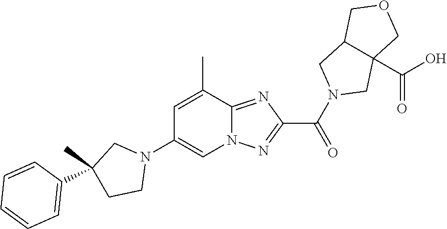 [SMILES: Cc1cc(N2CC[C@](C)(c3ccccc3)C2)cn2nc(C(=O)N3CC4COCC4(C(=O)O)C3)nc12] |
| 605 | 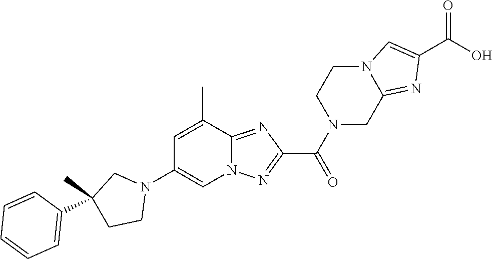 [SMILES: Cc1cc(N2CC[C@](C)(c3ccccc3)C2)cn2nc(C(=O)N3CCn4cc(C(=O)O)nc4C3)nc12] |
| 606 | 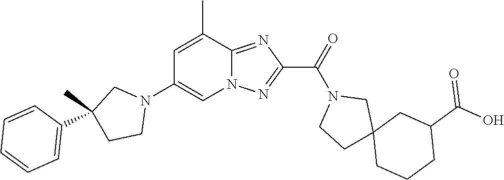 [SMILES: Cc1cc(N2CC[C@](C)(c3ccccc3)C2)cn2nc(C(=O)N3CCC4(CCCC(C(=O)O)C4)C3)nc12] |
| 609 | 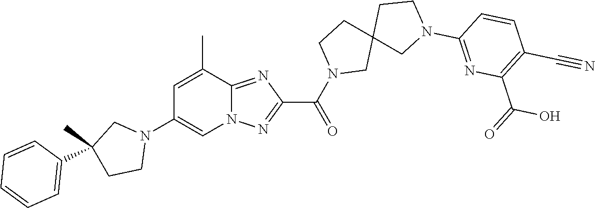 [SMILES: Cc1cc(N2CC[C@](C)(c3ccccc3)C2)cn2nc(C(=O)N3CCC4(CCN(c5ccc(C#N)c(C(=O)O)n5)C4)C3)nc12] |
| 610 | 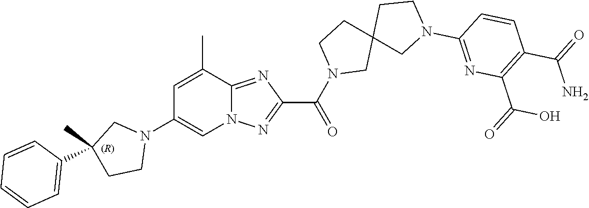 |
| 611 | 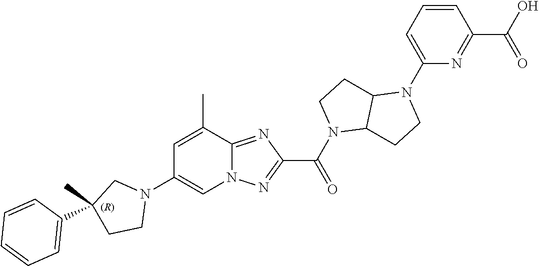 |
| 615 | 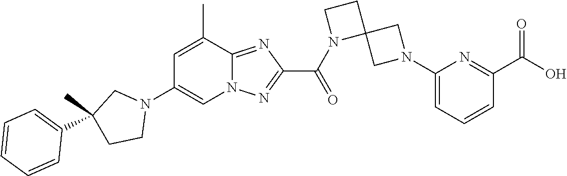 [SMILES: Cc1cc(N2CC[C@](C)(c3ccccc3)C2)cn2nc(C(=O)N3CCC34CN(c3cccc(C(=O)O)n3)C4)nc12] |
| 616 | 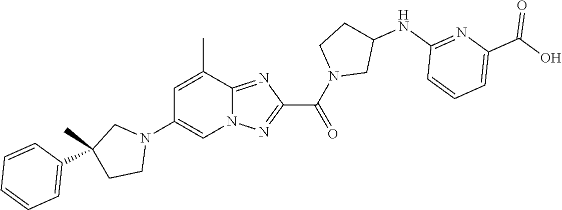 [SMILES: Cc1cc(N2CC[C@](C)(c3ccccc3)C2)cn2nc(C(=O)N3CCC(Nc4cccc(C(=O)O)n4)C3)nc12] |
| 618 | 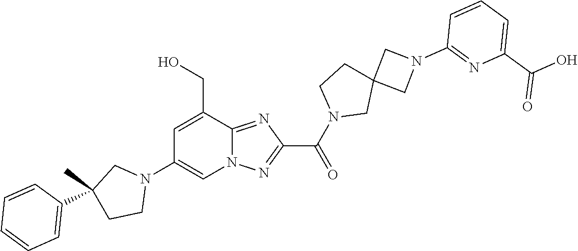 [SMILES: C[C@]1(c2ccccc2)CCN(c2cc(CO)c3nc(C(=O)N4CCC5(C4)CN(c4cccc(C(=O)O)n4)C5)nn3c2)C1] |
| 619 | 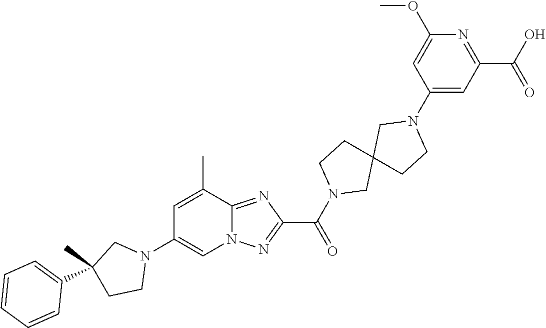 [SMILES: COc1cc(N2CCC3(CCN(C(=O)c4nc5c(C)cc(N6CC[C@](C)(c7ccccc7)C6)cn5n4)C3)C2)cc(C(=O)O)n1] |
| 621 | 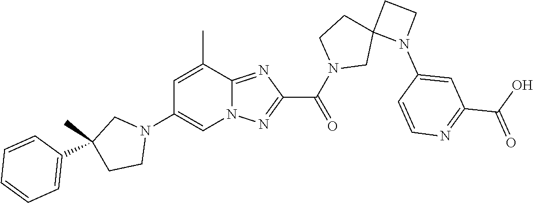 [SMILES: Cc1cc(N2CC[C@](C)(c3ccccc3)C2)cn2nc(C(=O)N3CCC4(CCN4c4ccnc(C(=O)O)c4)C3)nc12] |
| 622 | 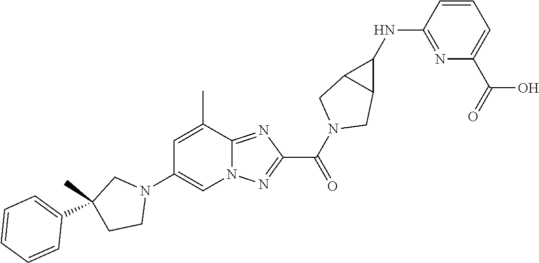 [SMILES: Cc1cc(N2CC[C@](C)(c3ccccc3)C2)cn2nc(C(=O)N3CC4C(C3)C4Nc3cccc(C(=O)O)n3)nc12] |
| 625 | 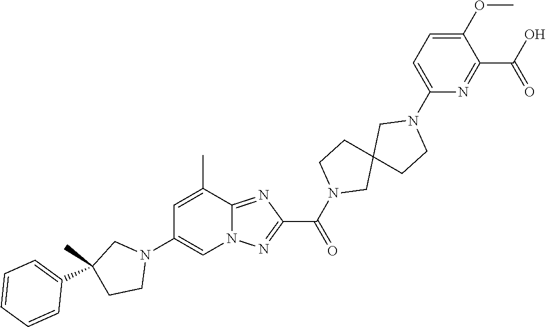 [SMILES: COc1ccc(N2CCC3(CCN(C(=O)c4nc5c(C)cc(N6CC[C@](C)(c7ccccc7)C6)cn5n4)C3)C2)nc1C(=O)O] |
| 626 | 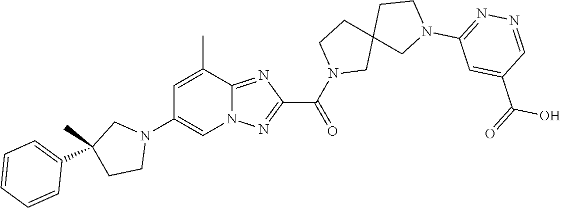 [SMILES: Cc1cc(N2CC[C@](C)(c3ccccc3)C2)cn2nc(C(=O)N3CCC4(CCN(c5cc(C(=O)O)cnn5)C4)C3)nc12] |
| 627 | 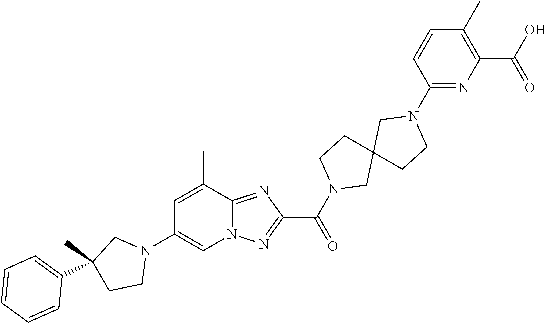 [SMILES: Cc1ccc(N2CCC3(CCN(C(=O)c4nc5c(C)cc(N6CC[C@](C)(c7ccccc7)C6)cn5n4)C3)C2)nc1C(=O)O] |
| 629 | 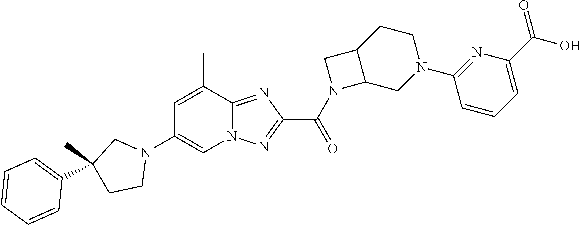 [SMILES: Cc1cc(N2CC[C@](C)(c3ccccc3)C2)cn2nc(C(=O)N3CC4CCN(c5cccc(C(=O)O)n5)CC43)nc12] |
| 630 | 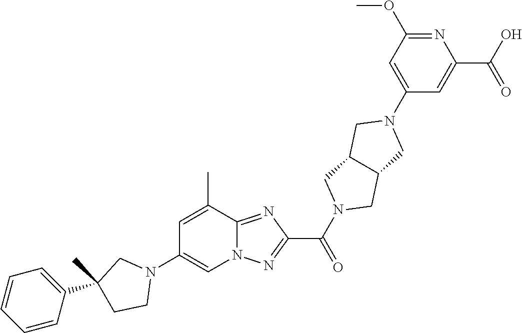 [SMILES: COc1cc(N2C[C@H]3CN(C(=O)c4nc5c(C)cc(N6CC[C@](C)(c7ccccc7)C6)cn5n4)C[C@H]3C2)cc(C(=O)O)n1] |
| 631 | 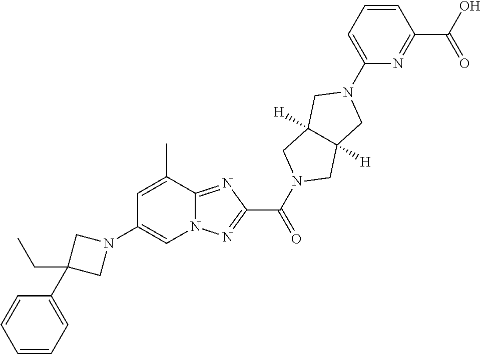 [SMILES: CCC1(c2ccccc2)CN(c2cc(C)c3nc(C(=O)N4C[C@@H]5CN(c6cccc(C(=O)O)n6)C[C@@H]5C4)nn3c2)C1] |
| 632 | 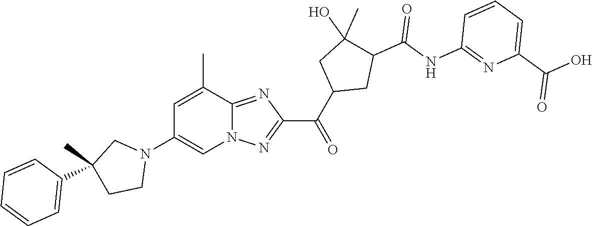 [SMILES: Cc1cc(N2CC[C@](C)(c3ccccc3)C2)cn2nc(C(=O)C3CC(C(=O)Nc4cccc(C(=O)O)n4)C(C)(O)C3)nc12] |
| 636 | 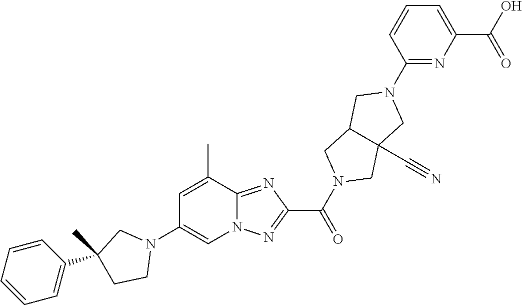 [SMILES: Cc1cc(N2CC[C@](C)(c3ccccc3)C2)cn2nc(C(=O)N3CC4CN(c5cccc(C(=O)O)n5)CC4(C#N)C3)nc12] |
| 637 | 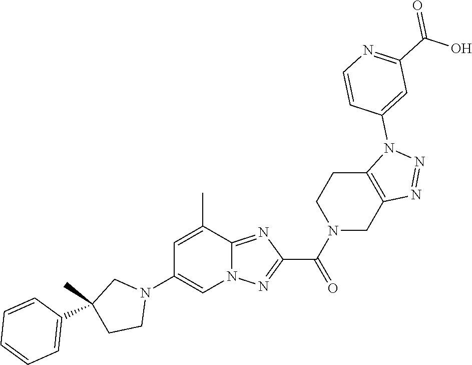 [SMILES: Cc1cc(N2CC[C@](C)(c3ccccc3)C2)cn2nc(C(=O)N3CCc4c(nnn4-c4ccnc(C(=O)O)c4)C3)nc12] |
| 638 | 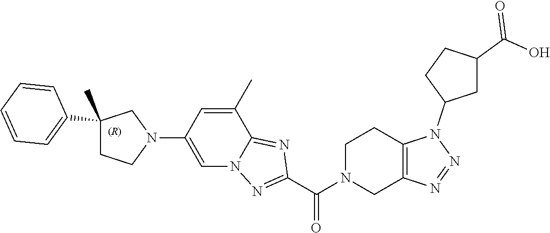 |
| 639 | 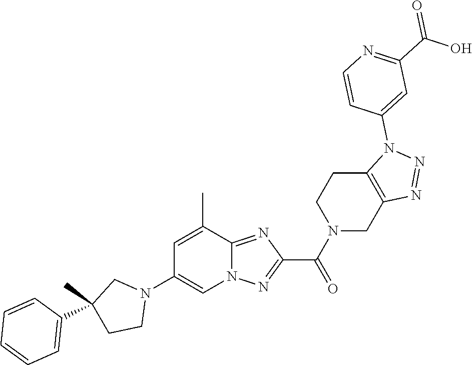 [SMILES: Cc1cc(N2CC[C@](C)(c3ccccc3)C2)cn2nc(C(=O)N3CCc4c(nnn4-c4ccnc(C(=O)O)c4)C3)nc12] |
| 640 | 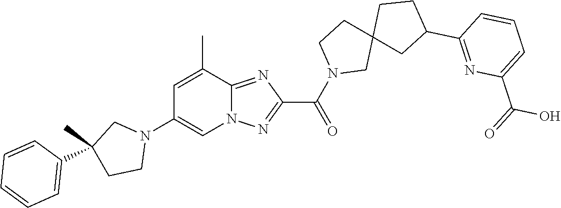 [SMILES: Cc1cc(N2CC[C@](C)(c3ccccc3)C2)cn2nc(C(=O)N3CCC4(CCC(c5cccc(C(=O)O)n5)C4)C3)nc12] |
| 641 | 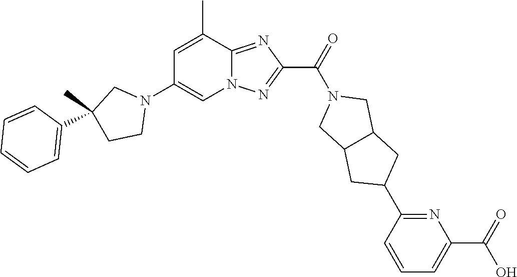 [SMILES: Cc1cc(N2CC[C@](C)(c3ccccc3)C2)cn2nc(C(=O)N3CC4CC(c5cccc(C(=O)O)n5)CC4C3)nc12] |
| 643 | 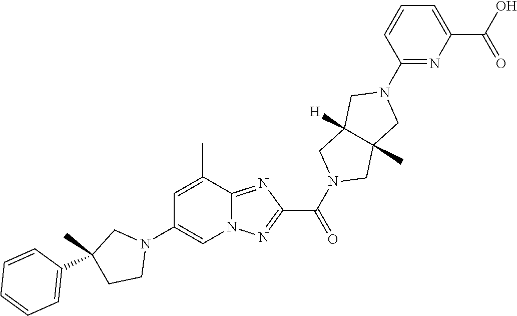 [SMILES: Cc1cc(N2CC[C@](C)(c3ccccc3)C2)cn2nc(C(=O)N3C[C@@H]4CN(c5cccc(C(=O)O)n5)C[C@]4(C)C3)nc12] |
| 644 | 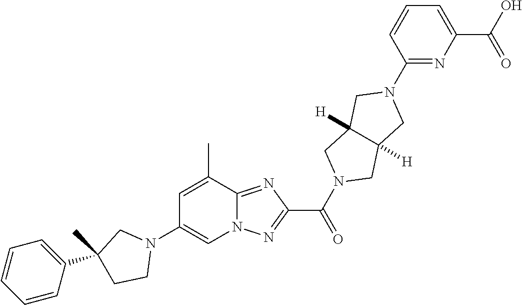 [SMILES: Cc1cc(N2CC[C@](C)(c3ccccc3)C2)cn2nc(C(=O)N3C[C@@H]4CN(c5cccc(C(=O)O)n5)C[C@H]4C3)nc12] |
| 645 | 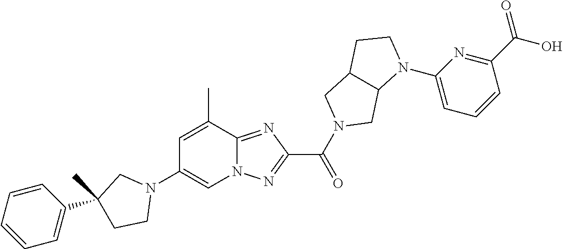 [SMILES: Cc1cc(N2CC[C@](C)(c3ccccc3)C2)cn2nc(C(=O)N3CC4CCN(c5cccc(C(=O)O)n5)C4C3)nc12] |
| 649 | 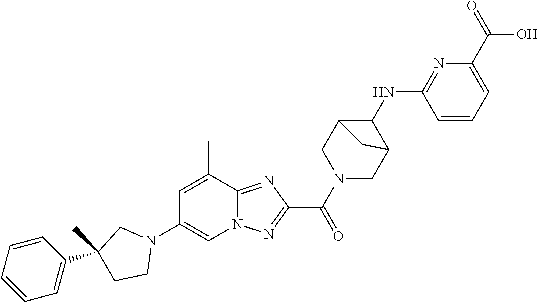 [SMILES: Cc1cc(N2CC[C@](C)(c3ccccc3)C2)cn2nc(C(=O)N3CC4CC(C3)C4Nc3cccc(C(=O)O)n3)nc12] |
| 650 | 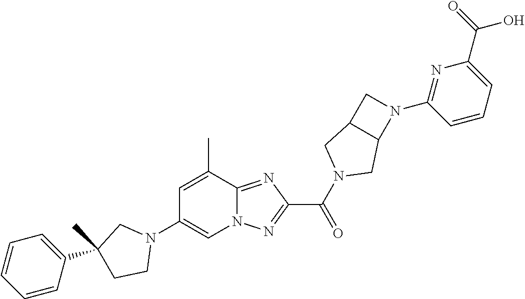 [SMILES: Cc1cc(N2CC[C@](C)(c3ccccc3)C2)cn2nc(C(=O)N3CC4CN(c5cccc(C(=O)O)n5)C4C3)nc12] |
| 651 | 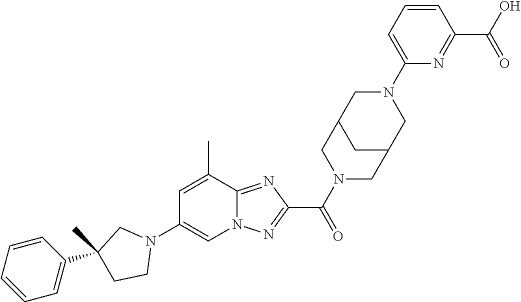 [SMILES: Cc1cc(N2CC[C@](C)(c3ccccc3)C2)cn2nc(C(=O)N3CC4CC(C3)CN(c3cccc(C(=O)O)n3)C4)nc12] |
| 653 | 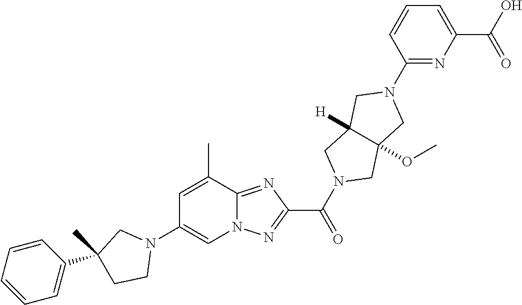 [SMILES: CO[C@]12CN(C(=O)c3nc4c(C)cc(N5CC[C@](C)(c6ccccc6)C5)cn4n3)C[C@@H]1CN(c1cccc(C(=O)O)n1)C2] |
| 657 | 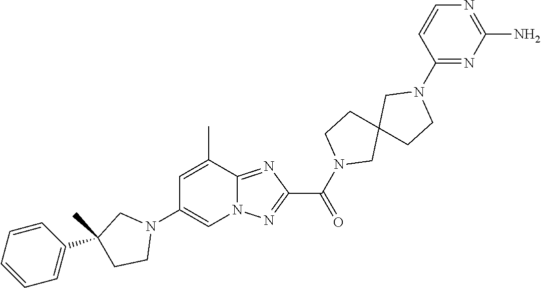 [SMILES: Cc1cc(N2CC[C@](C)(c3ccccc3)C2)cn2nc(C(=O)N3CCC4(CCN(c5ccnc(N)n5)C4)C3)nc12] |
| 659 | 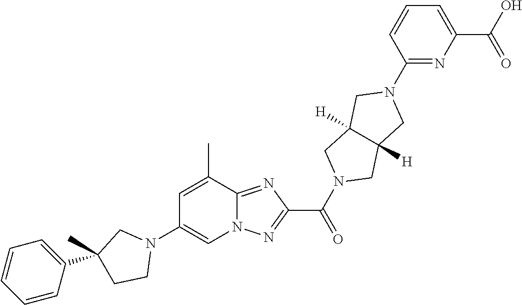 [SMILES: Cc1cc(N2CC[C@](C)(c3ccccc3)C2)cn2nc(C(=O)N3C[C@H]4CN(c5cccc(C(=O)O)n5)C[C@@H]4C3)nc12] |
| 665 | 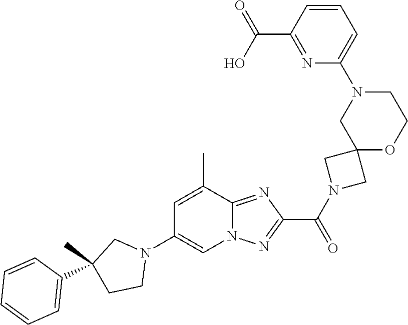 [SMILES: Cc1cc(N2CC[C@](C)(c3ccccc3)C2)cn2nc(C(=O)N3CC4(C3)CN(c3cccc(C(=O)O)n3)CCO4)nc12] |
| 670 | 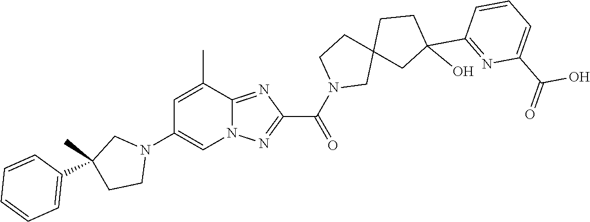 [SMILES: Cc1cc(N2CC[C@](C)(c3ccccc3)C2)cn2nc(C(=O)N3CCC4(CCC(O)(c5cccc(C(=O)O)n5)C4)C3)nc12] |
| 671 | 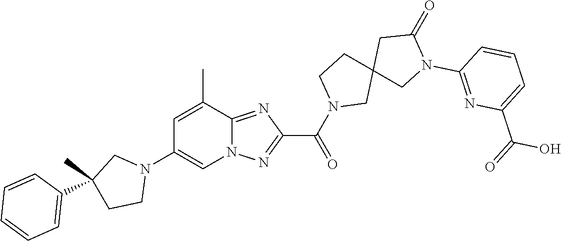 [SMILES: Cc1cc(N2CC[C@](C)(c3ccccc3)C2)cn2nc(C(=O)N3CCC4(CC(=O)N(c5cccc(C(=O)O)n5)C4)C3)nc12] |
| 672 | 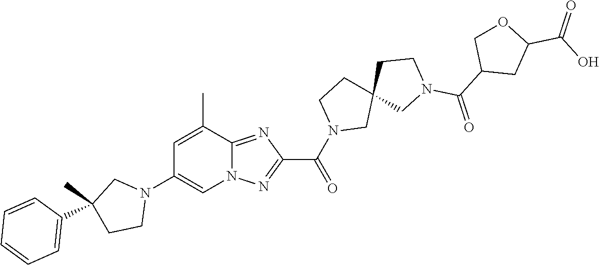 [SMILES: Cc1cc(N2CC[C@](C)(c3ccccc3)C2)cn2nc(C(=O)N3CC[C@@]4(CCN(C(=O)C5COC(C(=O)O)C5)C4)C3)nc12] |
| 674 | 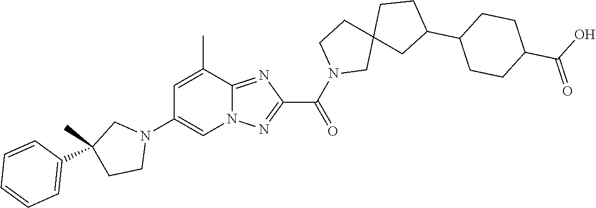 [SMILES: Cc1cc(N2CC[C@](C)(c3ccccc3)C2)cn2nc(C(=O)N3CCC4(CCC(C5CCC(C(=O)O)CC5)C4)C3)nc12] |
| 675 | 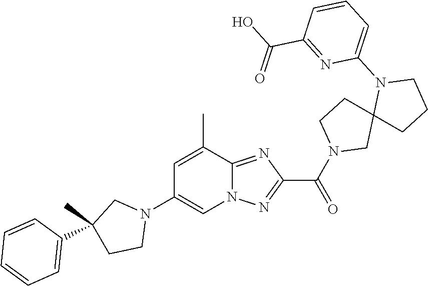 [SMILES: Cc1cc(N2CC[C@](C)(c3ccccc3)C2)cn2nc(C(=O)N3CCC4(CCCN4c4cccc(C(=O)O)n4)C3)nc12] |
| 687 | 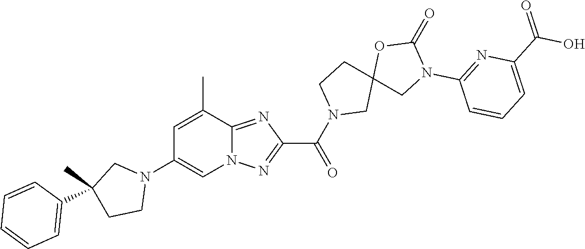 [SMILES: Cc1cc(N2CC[C@](C)(c3ccccc3)C2)cn2nc(C(=O)N3CCC4(C3)CN(c3cccc(C(=O)O)n3)C(=O)O4)nc12] |
| 688 | 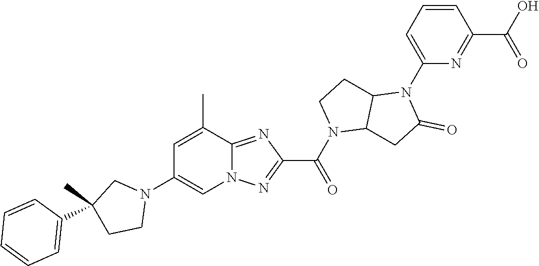 [SMILES: Cc1cc(N2CC[C@](C)(c3ccccc3)C2)cn2nc(C(=O)N3CCC4C3CC(=O)N4c3cccc(C(=O)O)n3)nc12] |
| 692 | 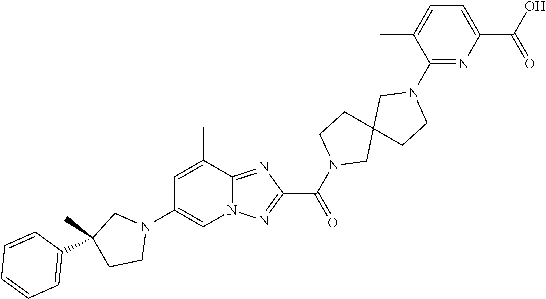 [SMILES: Cc1ccc(C(=O)O)nc1N1CCC2(CCN(C(=O)c3nc4c(C)cc(N5CC[C@](C)(c6ccccc6)C5)cn4n3)C2)C1] |
| 694 | 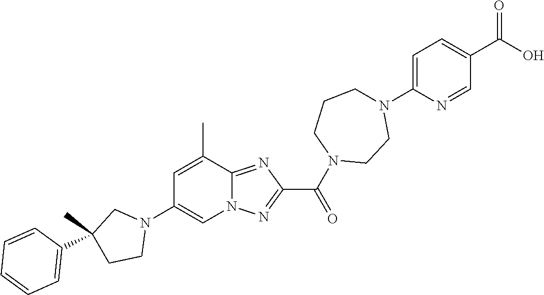 [SMILES: Cc1cc(N2CC[C@](C)(c3ccccc3)C2)cn2nc(C(=O)N3CCCN(c4ccc(C(=O)O)cn4)CC3)nc12] |
| 695 | 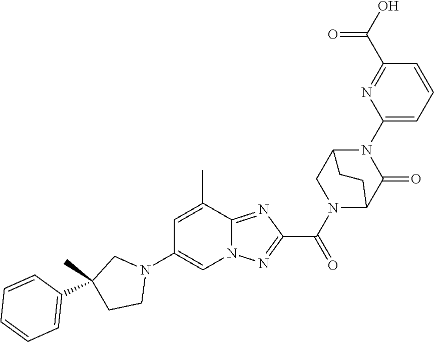 [SMILES: Cc1cc(N2CC[C@](C)(c3ccccc3)C2)cn2nc(C(=O)N3CC4CCC3C(=O)N4c3cccc(C(=O)O)n3)nc12] |
| 702 | 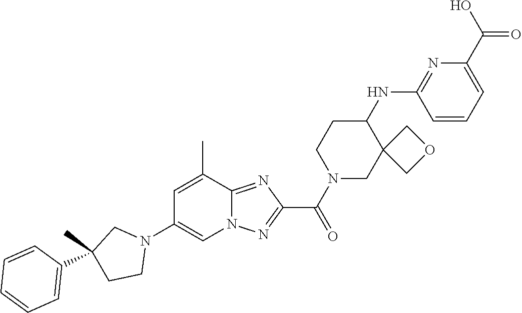 [SMILES: Cc1cc(N2CC[C@](C)(c3ccccc3)C2)cn2nc(C(=O)N3CCC(Nc4cccc(C(=O)O)n4)C4(COC4)C3)nc12] |
| 703 | 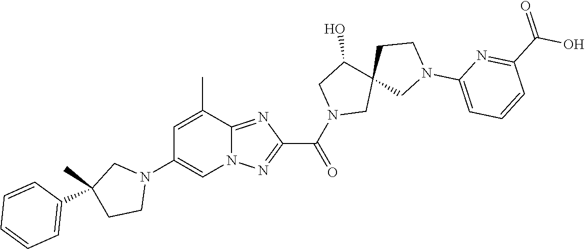 [SMILES: Cc1cc(N2CC[C@](C)(c3ccccc3)C2)cn2nc(C(=O)N3C[C@H](O)[C@@]4(CCN(c5cccc(C(=O)O)n5)C4)C3)nc12] |
| 704 | 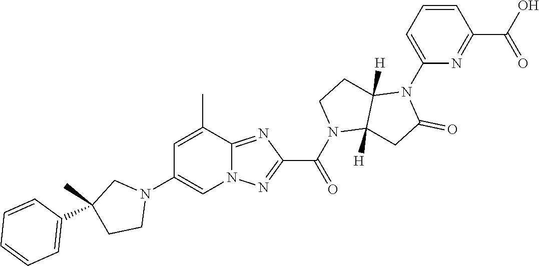 [SMILES: Cc1cc(N2CC[C@](C)(c3ccccc3)C2)cn2nc(C(=O)N3CC[C@H]4[C@@H]3CC(=O)N4c3cccc(C(=O)O)n3)nc12] |
| 705 | 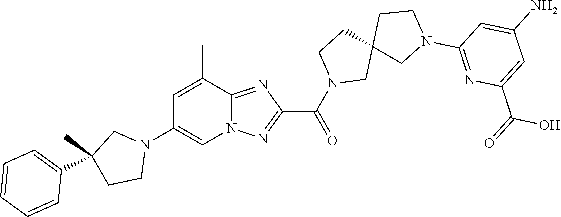 [SMILES: Cc1cc(N2CC[C@](C)(c3ccccc3)C2)cn2nc(C(=O)N3CC[C@@]4(CCN(c5cc(N)cc(C(=O)O)n5)C4)C3)nc12] |
| 706 | 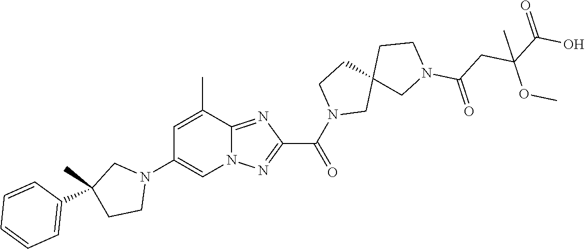 [SMILES: COC(C)(CC(=O)N1CC[C@@]2(CCN(C(=O)c3nc4c(C)cc(N5CC[C@](C)(c6ccccc6)C5)cn4n3)C2)C1)C(=O)O] |
| 707 | 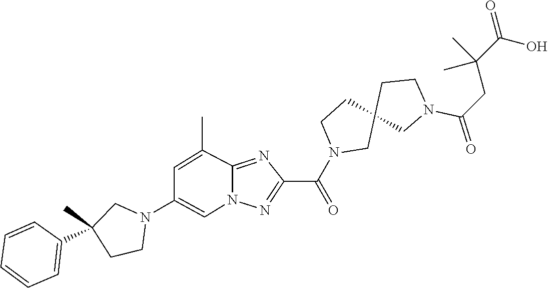 |
| 708 | 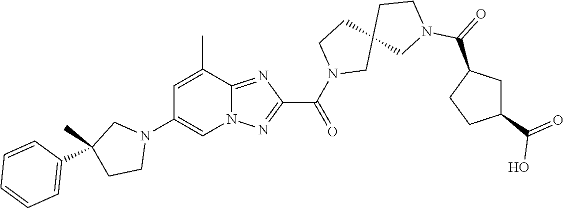 [SMILES: Cc1cc(N2CC[C@](C)(c3ccccc3)C2)cn2nc(C(=O)N3CC[C@@]4(CCN(C(=O)[C@@H]5CC[C@H](C(=O)O)C5)C4)C3)nc12] |
| 709 | 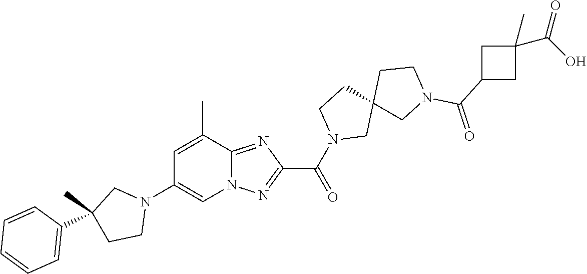 [SMILES: Cc1cc(N2CC[C@](C)(c3ccccc3)C2)cn2nc(C(=O)N3CC[C@@]4(CCN(C(=O)C5CC(C)(C(=O)O)C5)C4)C3)nc12] |
| 710 | 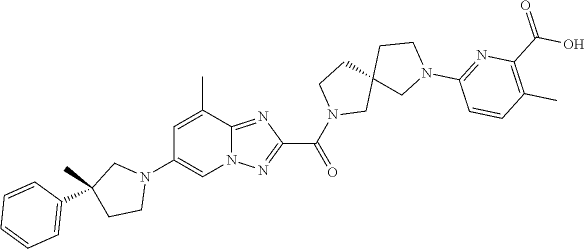 [SMILES: Cc1ccc(N2CC[C@@]3(CCN(C(=O)c4nc5c(C)cc(N6CC[C@](C)(c7ccccc7)C6)cn5n4)C3)C2)nc1C(=O)O] |
| 711 | 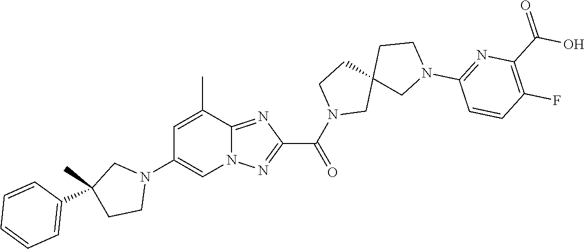 [SMILES: Cc1cc(N2CC[C@](C)(c3ccccc3)C2)cn2nc(C(=O)N3CC[C@@]4(CCN(c5ccc(F)c(C(=O)O)n5)C4)C3)nc12] |
| 712 | 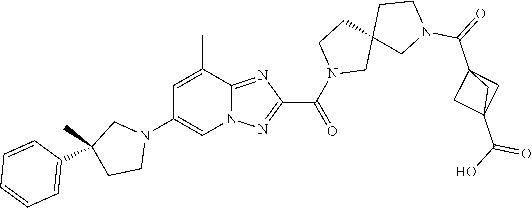 [SMILES: Cc1cc(N2CC[C@](C)(c3ccccc3)C2)cn2nc(C(=O)N3CC[C@@]4(CCN(C(=O)C56CC(C(=O)O)(C5)C6)C4)C3)nc12] |
| 713 | 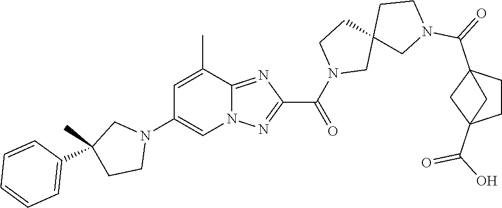 [SMILES: Cc1cc(N2CC[C@](C)(c3ccccc3)C2)cn2nc(C(=O)N3CC[C@@]4(CCN(C(=O)C56CCC(C(=O)O)(C5)C6)C4)C3)nc12] |
| 714 | 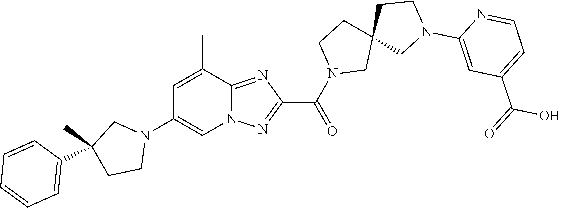 [SMILES: Cc1cc(N2CC[C@](C)(c3ccccc3)C2)cn2nc(C(=O)N3CC[C@@]4(CCN(c5cc(C(=O)O)ccn5)C4)C3)nc12] |
| 715 | 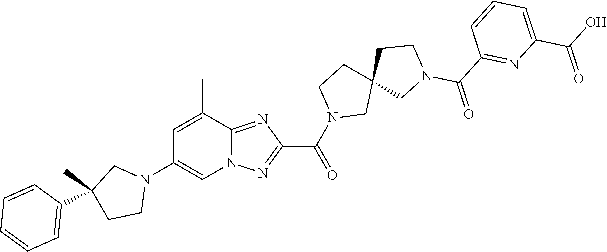 [SMILES: Cc1cc(N2CC[C@](C)(c3ccccc3)C2)cn2nc(C(=O)N3CC[C@@]4(CCN(C(=O)c5cccc(C(=O)O)n5)C4)C3)nc12] |
| 716 | 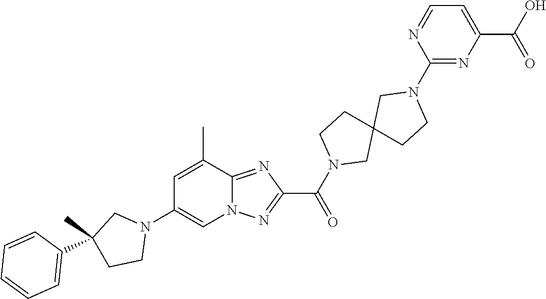 [SMILES: Cc1cc(N2CC[C@](C)(c3ccccc3)C2)cn2nc(C(=O)N3CCC4(CCN(c5nccc(C(=O)O)n5)C4)C3)nc12] |
| 717 | 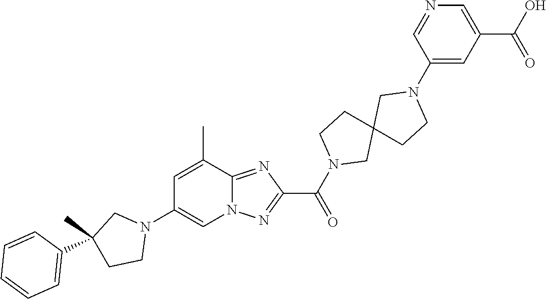 [SMILES: Cc1cc(N2CC[C@](C)(c3ccccc3)C2)cn2nc(C(=O)N3CCC4(CCN(c5cncc(C(=O)O)c5)C4)C3)nc12] |
| 718 | 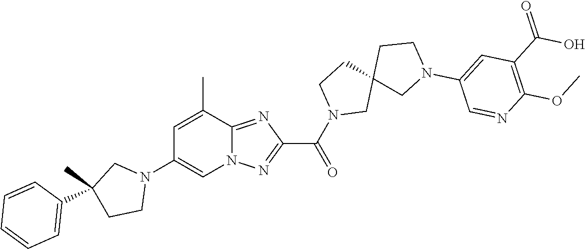 |
| 719 | 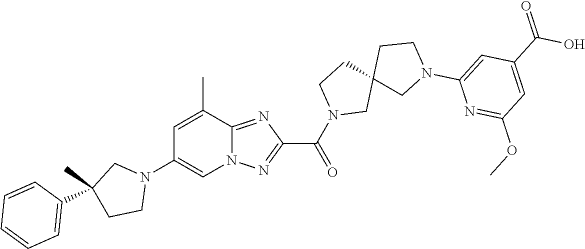 |
| 720 | 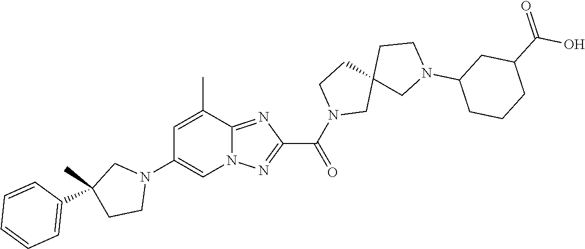 [SMILES: Cc1cc(N2CC[C@](C)(c3ccccc3)C2)cn2nc(C(=O)N3CC[C@@]4(CCN(C5CCCC(C(=O)O)C5)C4)C3)nc12] |
| 721 | 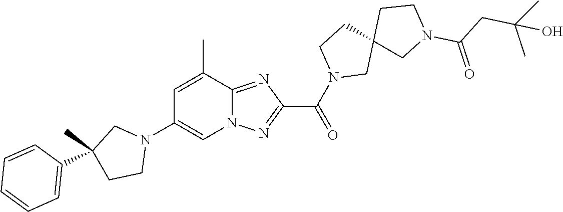 [SMILES: Cc1cc(N2CC[C@](C)(c3ccccc3)C2)cn2nc(C(=O)N3CC[C@@]4(CCN(C(=O)CC(C)(C)O)C4)C3)nc12] |
| 722 | 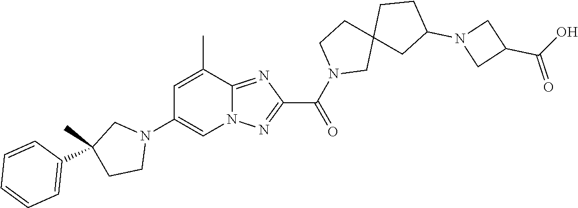 [SMILES: Cc1cc(N2CC[C@](C)(c3ccccc3)C2)cn2nc(C(=O)N3CCC4(CCC(N5CC(C(=O)O)C5)C4)C3)nc12] |
| 723 | 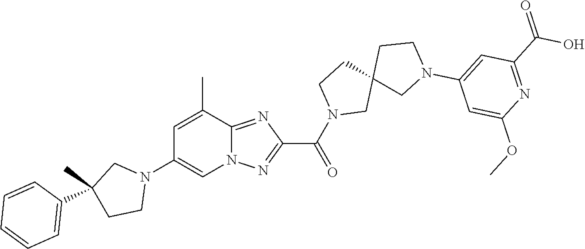 |
| 724 | 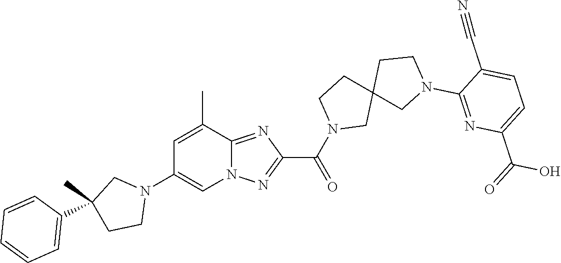 [SMILES: Cc1cc(N2CC[C@](C)(c3ccccc3)C2)cn2nc(C(=O)N3CCC4(CCN(c5nc(C(=O)O)ccc5C#N)C4)C3)nc12] |
| 725 | 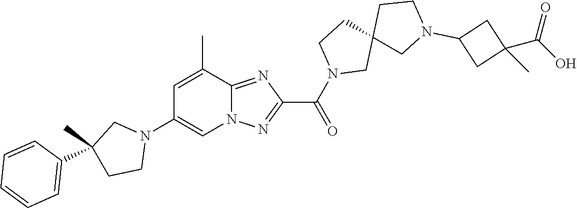 [SMILES: Cc1cc(N2CC[C@](C)(c3ccccc3)C2)cn2nc(C(=O)N3CC[C@@]4(CCN(C5CC(C)(C(=O)O)C5)C4)C3)nc12] |
| 726 | 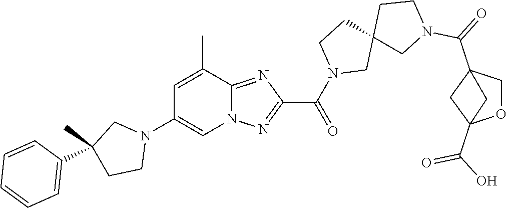 [SMILES: Cc1cc(N2CC[C@](C)(c3ccccc3)C2)cn2nc(C(=O)N3CC[C@@]4(CCN(C(=O)C56COC(C(=O)O)(C5)C6)C4)C3)nc12] |
| 727 | 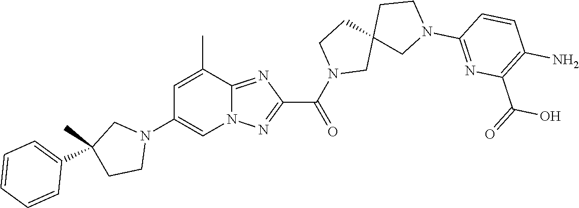 [SMILES: Cc1cc(N2CC[C@](C)(c3ccccc3)C2)cn2nc(C(=O)N3CC[C@@]4(CCN(c5ccc(N)c(C(=O)O)n5)C4)C3)nc12] |
| 729 | 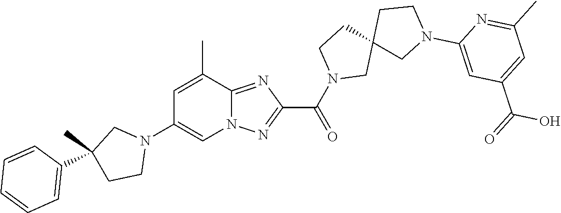 [SMILES: Cc1cc(C(=O)O)cc(N2CC[C@@]3(CCN(C(=O)c4nc5c(C)cc(N6CC[C@](C)(c7ccccc7)C6)cn5n4)C3)C2)n1] |
| 730 | 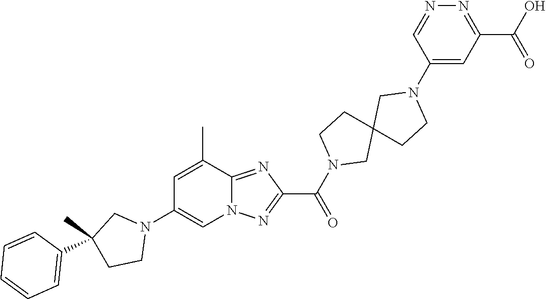 [SMILES: Cc1cc(N2CC[C@](C)(c3ccccc3)C2)cn2nc(C(=O)N3CCC4(CCN(c5cnnc(C(=O)O)c5)C4)C3)nc12] |
| 731 | 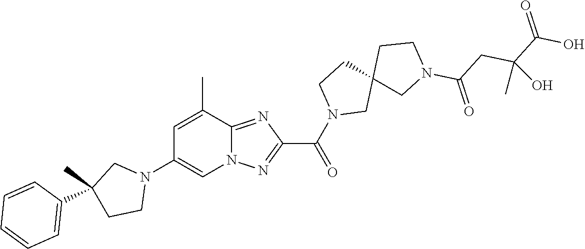 |
| 732 | 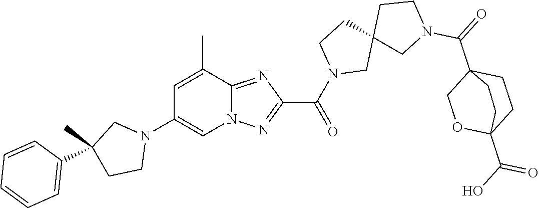 [SMILES: Cc1cc(N2CC[C@](C)(c3ccccc3)C2)cn2nc(C(=O)N3CC[C@@]4(CCN(C(=O)C56CCC(C(=O)O)(CC5)OC6)C4)C3)nc12] |
| 733 | 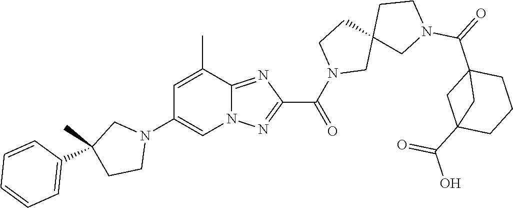 [SMILES: Cc1cc(N2CC[C@](C)(c3ccccc3)C2)cn2nc(C(=O)N3CC[C@@]4(CCN(C(=O)C56CCCC(C(=O)O)(C5)C6)C4)C3)nc12] |
| 734 | 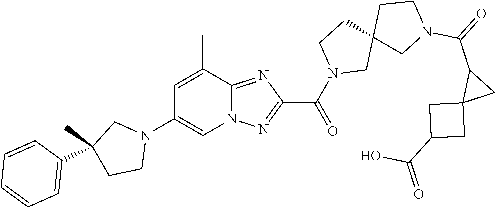 [SMILES: Cc1cc(N2CC[C@](C)(c3ccccc3)C2)cn2nc(C(=O)N3CC[C@@]4(CCN(C(=O)C5CC56CC(C(=O)O)C6)C4)C3)nc12] |
| 735 | 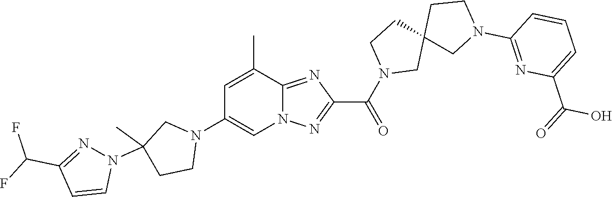 [SMILES: Cc1cc(N2CCC(C)(n3ccc(C(F)F)n3)C2)cn2nc(C(=O)N3CC[C@@]4(CCN(c5cccc(C(=O)O)n5)C4)C3)nc12] |
| 741 | 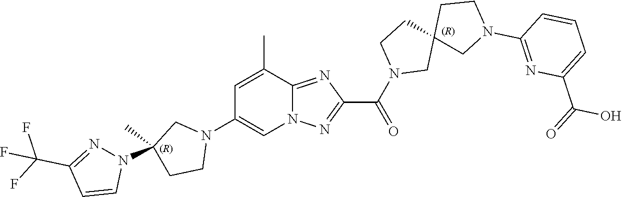 |
| 742 | 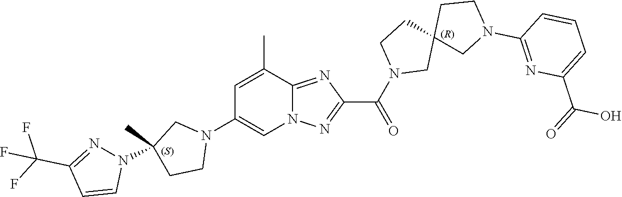 |
| 744 | 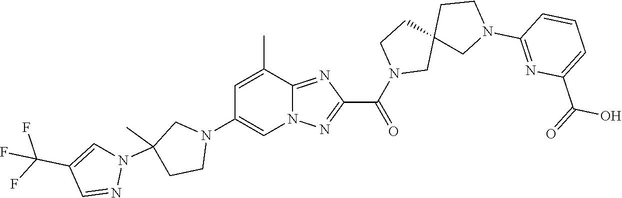 [SMILES: Cc1cc(N2CCC(C)(n3cc(C(F)(F)F)cn3)C2)cn2nc(C(=O)N3CC[C@@]4(CCN(c5cccc(C(=O)O)n5)C4)C3)nc12] |
| 745 | 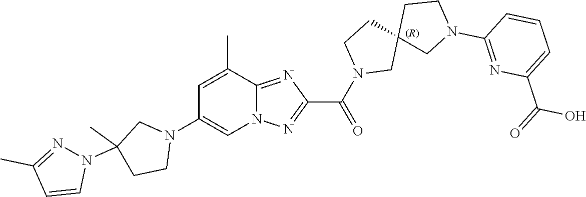 |
| 746 | 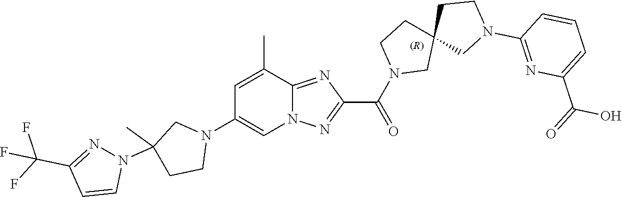 |
| 747 | 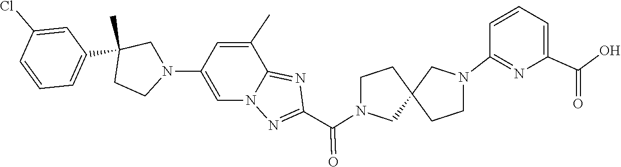 [SMILES: Cc1cc(N2CC[C@](C)(c3cccc(Cl)c3)C2)cn2nc(C(=O)N3CC[C@@]4(CCN(c5cccc(C(=O)O)n5)C4)C3)nc12] |
| 748 | 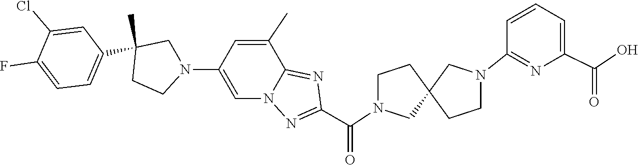 [SMILES: Cc1cc(N2CC[C@](C)(c3ccc(F)c(Cl)c3)C2)cn2nc(C(=O)N3CC[C@@]4(CCN(c5cccc(C(=O)O)n5)C4)C3)nc12] |
| 749 | 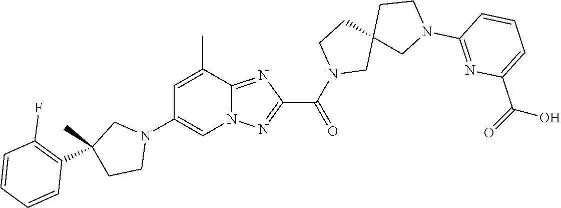 [SMILES: Cc1cc(N2CC[C@](C)(c3ccccc3F)C2)cn2nc(C(=O)N3CC[C@@]4(CCN(c5cccc(C(=O)O)n5)C4)C3)nc12] |
| 750 | 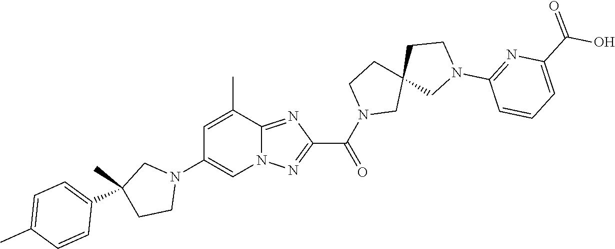 [SMILES: Cc1ccc([C@@]2(C)CCN(c3cc(C)c4nc(C(=O)N5CC[C@@]6(CCN(c7cccc(C(=O)O)n7)C6)C5)nn4c3)C2)cc1] |
| 751 | 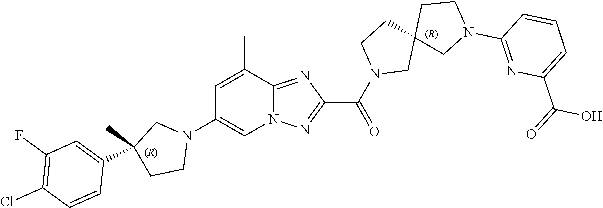 |
| 752 | 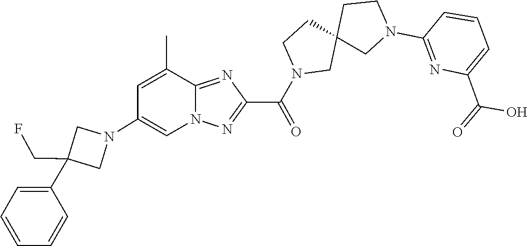 [SMILES: Cc1cc(N2CC(CF)(c3ccccc3)C2)cn2nc(C(=O)N3CC[C@@]4(CCN(c5cccc(C(=O)O)n5)C4)C3)nc12] |
| 753 | 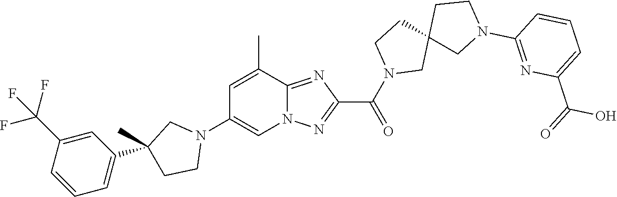 [SMILES: Cc1cc(N2CC[C@](C)(c3cccc(C(F)(F)F)c3)C2)cn2nc(C(=O)N3CC[C@@]4(CCN(c5cccc(C(=O)O)n5)C4)C3)nc12] |
| 754 | 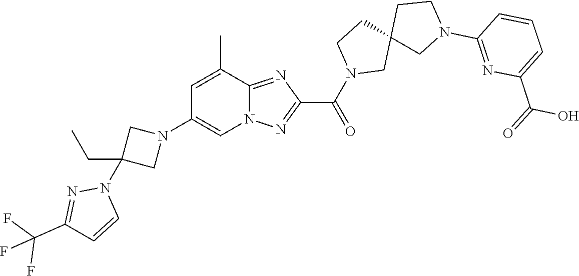 [SMILES: CCC1(n2ccc(C(F)(F)F)n2)CN(c2cc(C)c3nc(C(=O)N4CC[C@@]5(CCN(c6cccc(C(=O)O)n6)C5)C4)nn3c2)C1] |
| 755 | 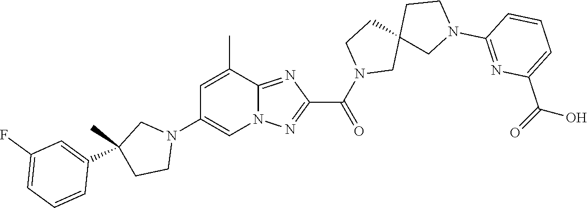 [SMILES: Cc1cc(N2CC[C@](C)(c3cccc(F)c3)C2)cn2nc(C(=O)N3CC[C@@]4(CCN(c5cccc(C(=O)O)n5)C4)C3)nc12] |

TABLE 4

| Cmpd | |
|---|---|
| # | Structure |
| 61 | 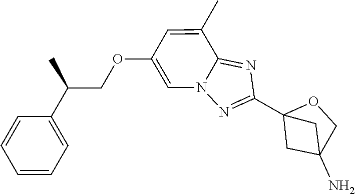 [SMILES: Cc1cc(OC[C@H](C)c2ccccc2)cn2nc(C34CC(N)(CO3)C4)nc12] |
| 98 | 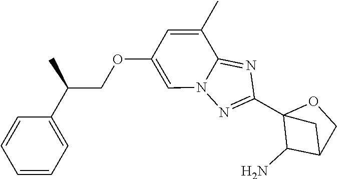 [SMILES: Cc1cc(OC[C@H](C)c2ccccc2)cn2nc(C34CC(CO3)C4N)nc12] |
| 303 | 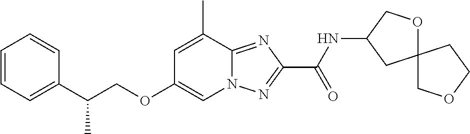 [SMILES: Cc1cc(OC[C@H](C)c2ccccc2)cn2nc(C(=O)NC3COC4(CCOC4)C3)nc12] |
| 26 | 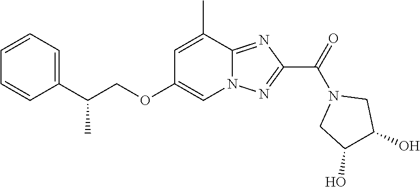 [SMILES: Cc1cc(OC[C@H](C)c2ccccc2)cn2nc(C(=O)N3C[C@@H](O)[C@@H](O)C3)nc12] |
| 304 | 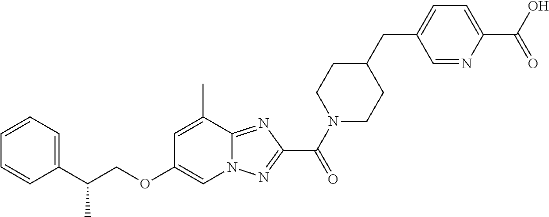 [SMILES: Cc1cc(OC[C@H](C)c2ccccc2)cn2nc(C(=O)N3CCC(Cc4ccc(C(=O)O)nc4)CC3)nc12] |
| 305 | 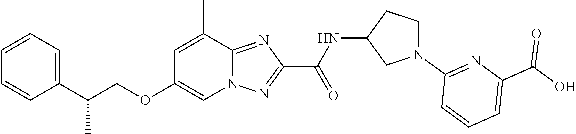 [SMILES: Cc1cc(OC[C@H](C)c2ccccc2)cn2nc(C(=O)NC3CCN(c4cccc(C(=O)O)n4)C3)nc12] |
| 306 | 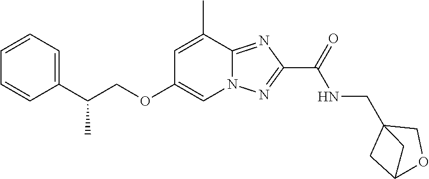 [SMILES: Cc1cc(OC[C@H](C)c2ccccc2)cn2nc(C(=O)NCC34COC(C3)C4)nc12] |
| 307 | 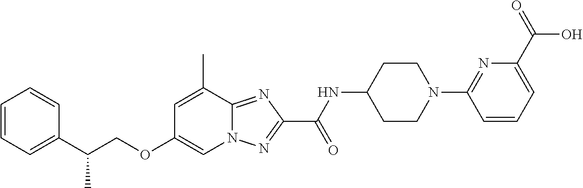 [SMILES: Cc1cc(OC[C@H](C)c2ccccc2)cn2nc(C(=O)NC3CCN(c4cccc(C(=O)O)n4)CC3)nc12] |
| 308 | 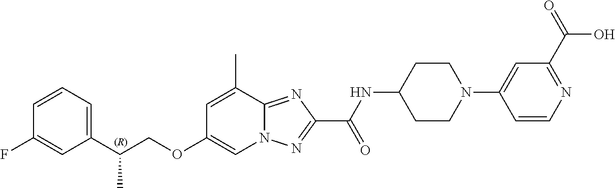 |
| 309 | 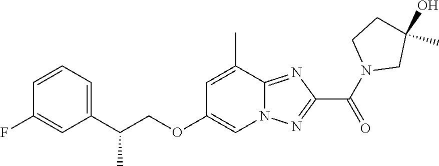 [SMILES: Cc1cc(OC[C@H](C)c2cccc(F)c2)cn2nc(C(=O)N3CC[C@@](C)(O)C3)nc12] |
| 310 | 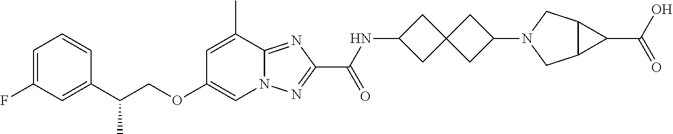 [SMILES: Cc1cc(OC[C@H](C)c2cccc(F)c2)cn2nc(C(=O)NC3CC4(C3)CC(N3CC5C(C3)C5C(=O)O)C4)nc12] |
| 311 | 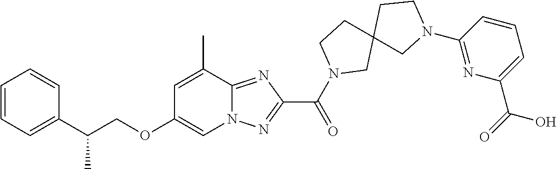 [SMILES: Cc1cc(OC[C@H](C)c2ccccc2)cn2nc(C(=O)N3CCC4(CCN(c5cccc(C(=O)O)n5)C4)C3)nc12] |
| 312 | 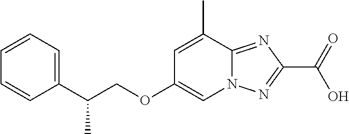 [SMILES: Cc1cc(OC[C@H](C)c2ccccc2)cn2nc(C(=O)O)nc12] |
| 313 | 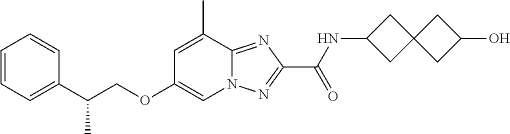 [SMILES: Cc1cc(OC[C@H](C)c2ccccc2)cn2nc(C(=O)NC3CC4(CC(O)C4)C3)nc12] |
| 314 | 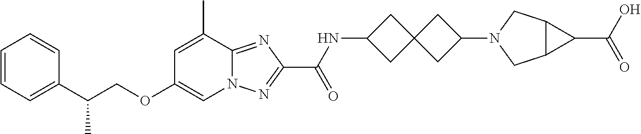 [SMILES: Cc1cc(OC[C@H](C)c2ccccc2)cn2nc(C(=O)NC3CC4(C3)CC(N3CC5C(C3)C5C(=O)O)C4)nc12] |
| 315 | 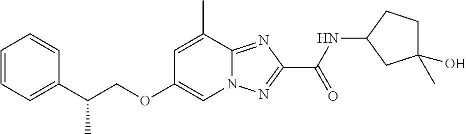 [SMILES: Cc1cc(OC[C@H](C)c2ccccc2)cn2nc(C(=O)NC3CCC(C)(O)C3)nc12] |
| 316 | 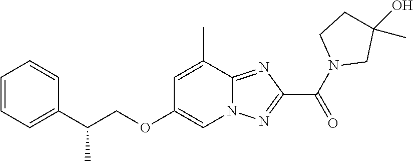 [SMILES: Cc1cc(OC[C@H](C)c2ccccc2)cn2nc(C(=O)N3CCC(C)(O)C3)nc12] |
| 16 | 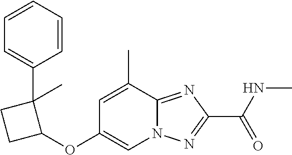 [SMILES: CNC(=O)c1nc2c(C)cc(OC3CCC3(C)c3ccccc3)cn2n1] |
| 24 | 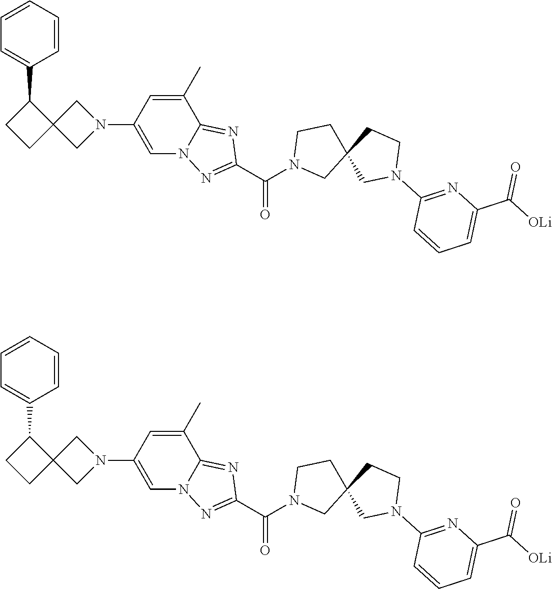 |
| 24′ | 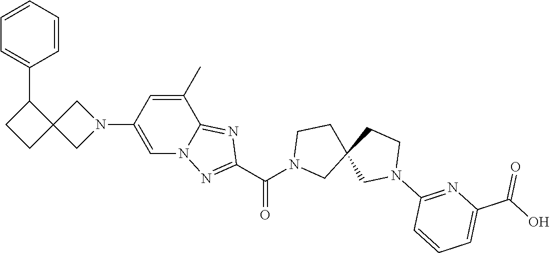 [SMILES: Cc1cc(N2CC3(CCC3c3ccccc3)C2)cn2nc(C(=O)N3CC[C@@]4(CCN(c5cccc(C(=O)O)n5)C4)C3)nc12] |
| 317 | 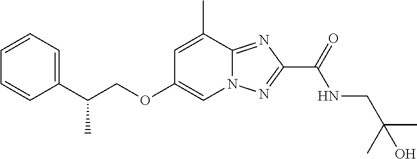 [SMILES: Cc1cc(OC[C@H](C)c2ccccc2)cn2nc(C(=O)NCC(C)(C)O)nc12] |
| 319 | 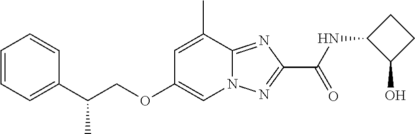 [SMILES: Cc1cc(OC[C@H](C)c2ccccc2)cn2nc(C(=O)N[C@@H]3CC[C@H]3O)nc12] |
| 320 | 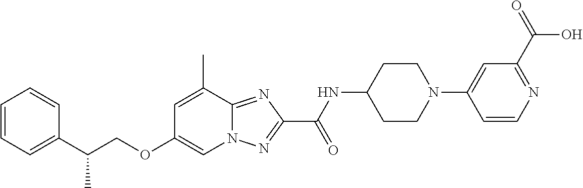 [SMILES: Cc1cc(OC[C@H](C)c2ccccc2)cn2nc(C(=O)NC3CCN(c4ccnc(C(=O)O)c4)CC3)nc12] |
| 321 | 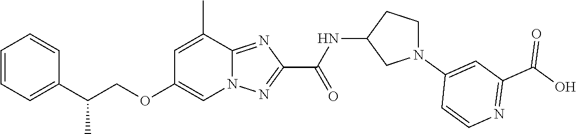 [SMILES: Cc1cc(OC[C@H](C)c2ccccc2)cn2nc(C(=O)NC3CCN(c4ccnc(C(=O)O)c4)C3)nc12] |
| 322 | 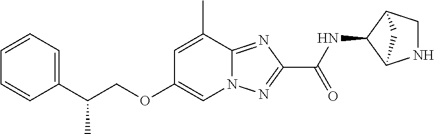 [SMILES: Cc1cc(OC[C@H](C)c2ccccc2)cn2nc(C(=O)N[C@H]3[C@H]4CN[C@@H]3C4)nc12] |
| 412 | 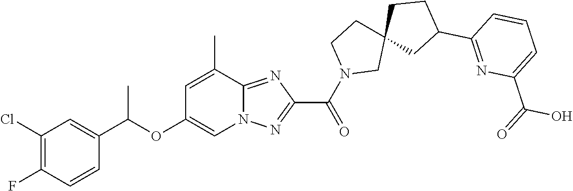 |
| 436 | 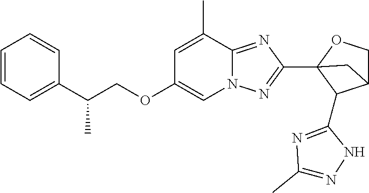 [SMILES: Cc1n[nH]c(C2C3COC2(c2nc4c(C)cc(OC[C@H](C)c5ccccc5)cn4n2)C3)n1] |
| 437 | 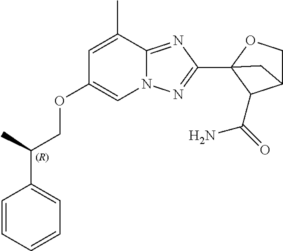 |
| 486 | 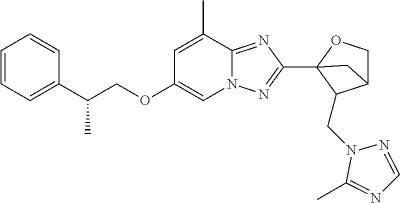 [SMILES: Cc1cc(OC[C@H](C)c2ccccc2)cn2nc(C34CC(CO3)C4Cn3ncnc3C)nc12] |
| 487 | 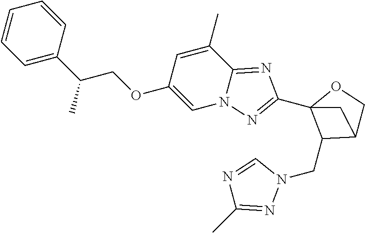 [SMILES: Cc1ncn(CC2C3COC2(c2nc4c(C)cc(OC[C@H](C)c5ccccc5)cn4n2)C3)n1] |
| 17 | 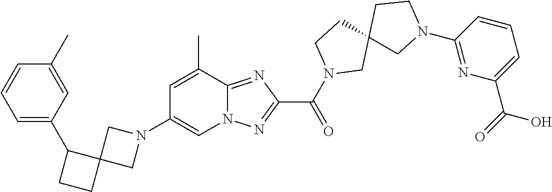 [SMILES: Cc1cccc(C2CCC23CN(c2cc(C)c4nc(C(=O)N5CC[C@@]6(CCN(c7cccc(C(=O)O)n7)C6)C5)nn4c2)C3)c1] |
| 291 | 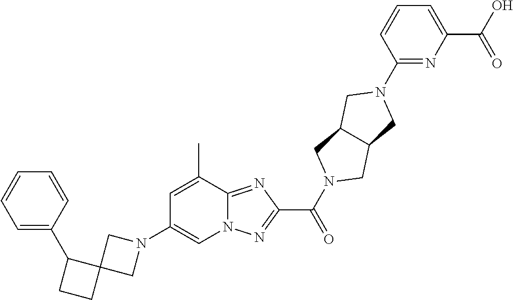 [SMILES: Cc1cc(N2CC3(CCC3c3ccccc3)C2)cn2nc(C(=O)N3C[C@@H]4CN(c5cccc(C(=O)O)n5)C[C@@H]4C3)nc12] |
| 512 | 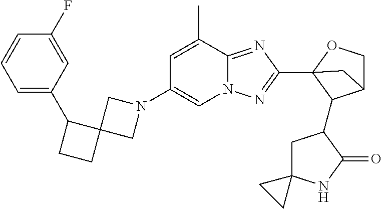 [SMILES: Cc1cc(N2CC3(CCC3c3cccc(F)c3)C2)cn2nc(C34CC(CO3)C4C3CC4(CC4)NC3=O)nc12] |
| 578 | 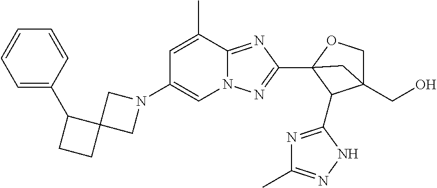 [SMILES: Cc1n[nH]c(C2C3(CO)COC2(c2nc4c(C)cc(N5CC6(CCC6c6ccccc6)C5)cn4n2)C3)n1] |
| 736 | 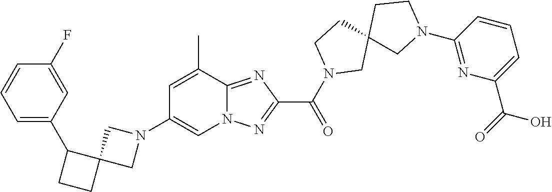 |
| 737 | 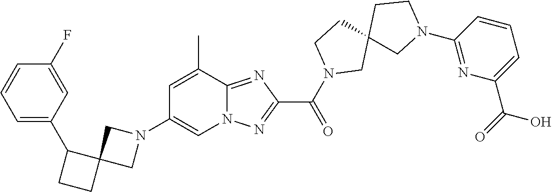 |
| 738 | 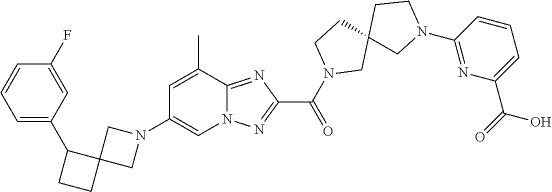 [SMILES: Cc1cc(N2CC3(CCC3c3cccc(F)c3)C2)cn2nc(C(=O)N3CC[C@@]4(CCN(c5cccc(C(=O)O)n5)C4)C3)nc12] |
| 739 | 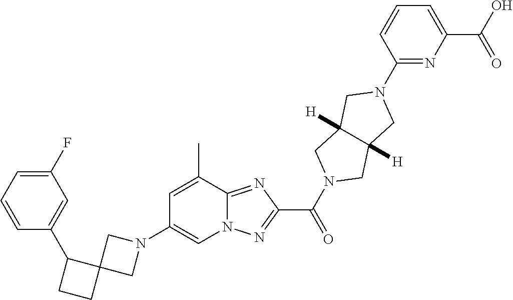 [SMILES: Cc1cc(N2CC3(CCC3c3cccc(F)c3)C2)cn2nc(C(=O)N3C[C@@H]4CN(c5cccc(C(=O)O)n5)C[C@@H]4C3)nc12] |
| 740 | 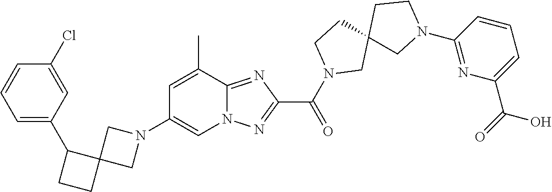 [SMILES: Cc1cc(N2CC3(CCC3c3cccc(Cl)c3)C2)cn2nc(C(=O)N3CC[C@@]4(CCN(c5cccc(C(=O)O)n5)C4)C3)nc12] |

TABLE 5

| Cmpd | |
|---|---|
| # | Structure |
| 147 | 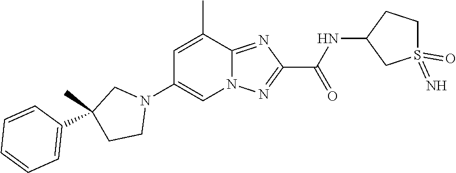 [SMILES: Cc1cc(N2CC[C@](C)(c3ccccc3)C2)cn2nc(C(=O)NC3CCS(=N)(=O)C3)nc12] |
| 168 | 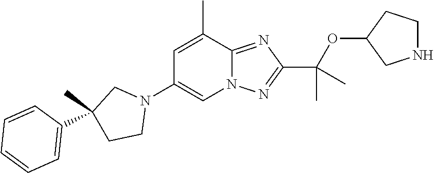 [SMILES: Cc1cc(N2CC[C@](C)(c3ccccc3)C2)cn2nc(C(C)(C)OC3CCNC3)nc12] |
| 2 | 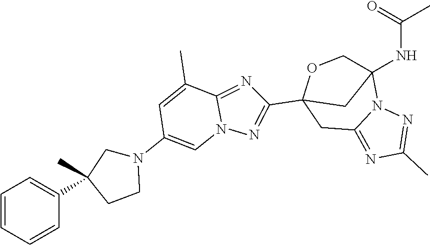 [SMILES: CC(=O)NC12COC(c3nc4c(C)cc(N5CC[C@](C)(c6ccccc6)C5)cn4n3)(Cc3nc(C)nn31)C2] |
| 47 | 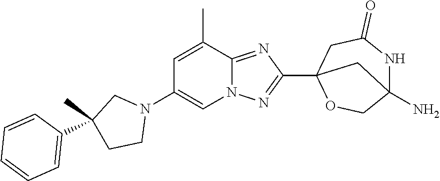 [SMILES: Cc1cc(N2CC[C@](C)(c3ccccc3)C2)cn2nc(C34CC(=O)NC(N)(CO3)C4)nc12] |
| 64 | 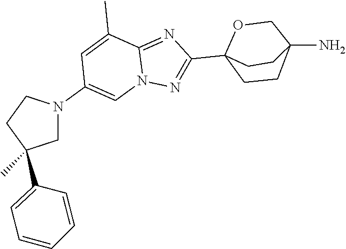 [SMILES: Cc1cc(N2CC[C@](C)(c3ccccc3)C2)cn2nc(C34CCC(N)(CC3)CO4)nc12] |
| 534 | 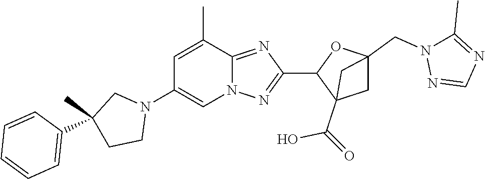 [SMILES: Cc1cc(N2CC[C@](C)(c3ccccc3)C2)cn2nc(C3OC4(Cn5ncnc5C)CC3(C(=O)O)C4)nc12] |
| 535 | 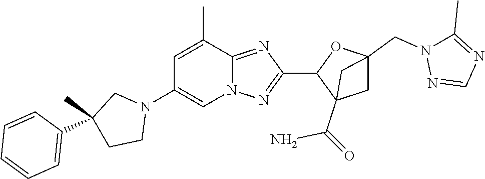 [SMILES: Cc1cc(N2CC[C@](C)(c3ccccc3)C2)cn2nc(C3OC4(Cn5ncnc5C)CC3(C(N)=O)C4)nc12] |
| 12 | 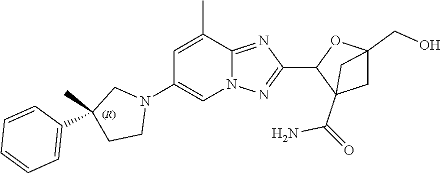 |
| 94 | 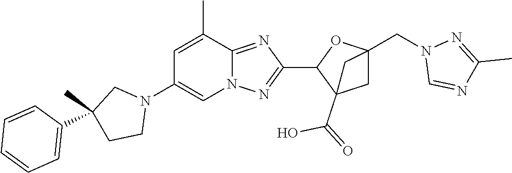 [SMILES: Cc1ncn(CC23CC(C(=O)O)(C2)C(c2nc4c(C)cc(N5CC[C@](C)(c6ccccc6)C5)cn4n2)O3)n1] |
| 95 | 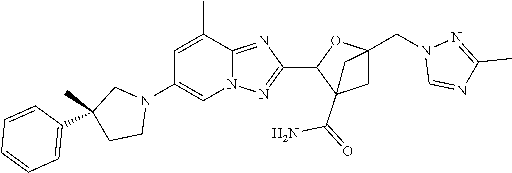 [SMILES: Cc1ncn(CC23CC(C(N)=O)(C2)C(c2nc4c(C)cc(N5CC[C@](C)(c6ccccc6)C5)cn4n2)O3)n1] |
| 474 | 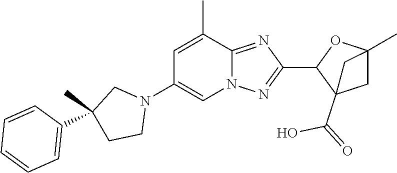 [SMILES: Cc1cc(N2CC[C@](C)(c3ccccc3)C2)cn2nc(C3OC4(C)CC3(C(=O)O)C4)nc12] |
| 475 | 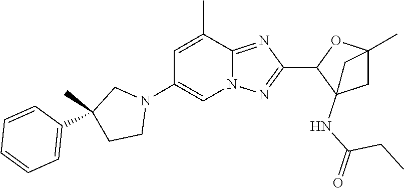 [SMILES: CCC(=O)NC12CC(C)(C1)OC2c1nc2c(C)cc(N3CC[C@](C)(c4ccccc4)C3)cn2n1] |
| 476 | 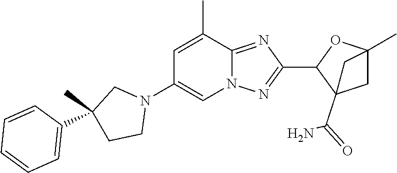 [SMILES: Cc1cc(N2CC[C@](C)(c3ccccc3)C2)cn2nc(C3OC4(C)CC3(C(N)=O)C4)nc12] |
| 27 | 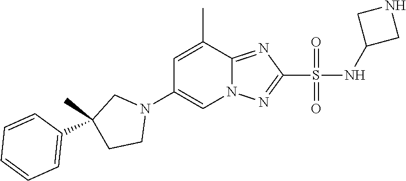 [SMILES: Cc1cc(N2CC[C@](C)(c3ccccc3)C2)cn2nc(S(=O)(=O)NC3CNC3)nc12] |
| 323 | 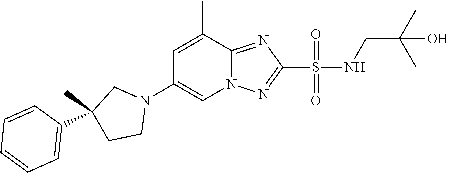 [SMILES: Cc1cc(N2CC[C@](C)(c3ccccc3)C2)cn2nc(S(=O)(=O)NCC(C)(C)O)nc12] |
| 324 | 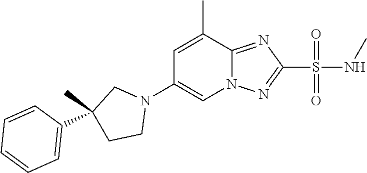 [SMILES: CNS(=O)(=O)c1nc2c(C)cc(N3CC[C@](C)(c4ccccc4)C3)cn2n1] |
| 325 | 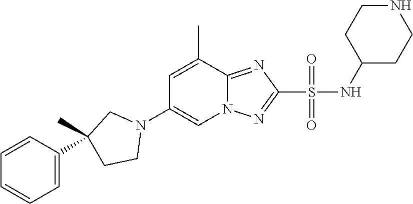 [SMILES: Cc1cc(N2CC[C@](C)(c3ccccc3)C2)cn2nc(S(=O)(=O)NC3CCNCC3)nc12] |
| 326 | 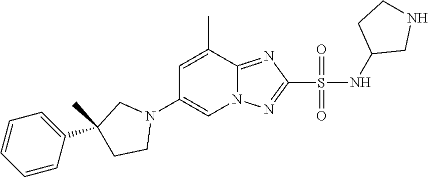 [SMILES: Cc1cc(N2CC[C@](C)(c3ccccc3)C2)cn2nc(S(=O)(=O)NC3CCNC3)nc12] |
| 29 | 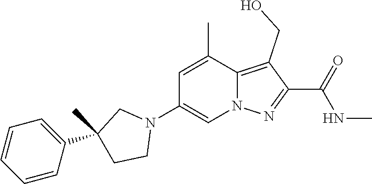 [SMILES: CNC(=O)c1nn2cc(N3CC[C@](C)(c4ccccc4)C3)cc(C)c2c1CO] |
| 22 | 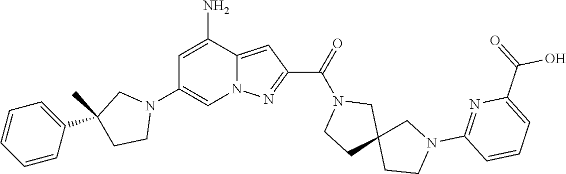 [SMILES: C[C@]1(c2ccccc2)CCN(c2cc(N)c3cc(C(=O)N4CC[C@@]5(CCN(c6cccc(C(=O)O)n6)C5)C4)nn3c2)C1] |
| 57 | 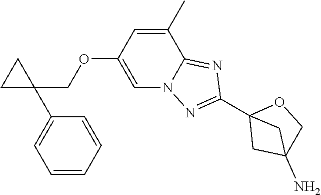 [SMILES: Cc1cc(OCC2(c3ccccc3)CC2)cn2nc(C34CC(N)(CO3)C4)nc12] |
| 756 | 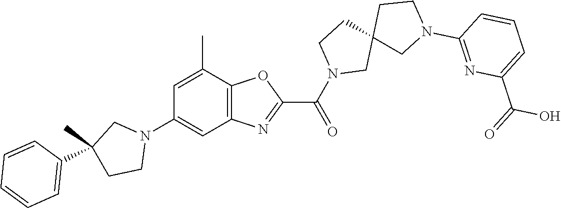 [SMILES: Cc1cc(N2CC[C@](C)(c3ccccc3)C2)cc2nc(C(=O)N3CC[C@@]4(CCN(c5cccc(C(=O)O)n5)C4)C3)oc12] |
| 333 | 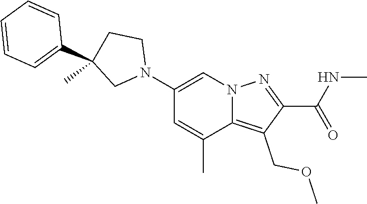 [SMILES: CNC(=O)c1nn2cc(N3CC[C@](C)(c4ccccc4)C3)cc(C)c2c1COC] |
| 39 | 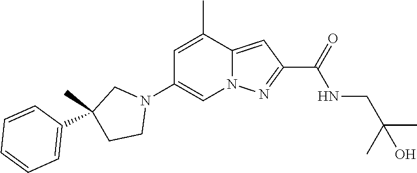 [SMILES: Cc1cc(N2CC[C@](C)(c3ccccc3)C2)cn2nc(C(=O)NCC(C)(C)O)cc12] |
| 433 | 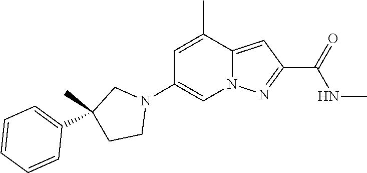 [SMILES: CNC(=O)c1cc2c(C)cc(N3CC[C@](C)(c4ccccc4)C3)cn2n1] |
| 434 | 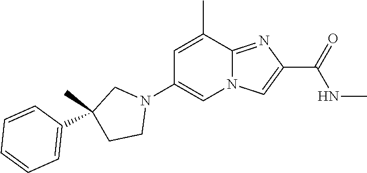 [SMILES: CNC(=O)c1cn2cc(N3CC[C@](C)(c4ccccc4)C3)cc(C)c2n1] |
| 435 | 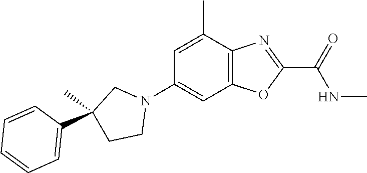 [SMILES: CNC(=O)c1nc2c(C)cc(N3CC[C@@](C)(c4ccccc4)C3)cc2o1] |
| 292 | 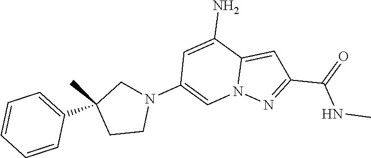 [SMILES: CNC(=O)c1cc2c(N)cc(N3CC[C@](C)(c4ccccc4)C3)cn2n1] |
| 40 | 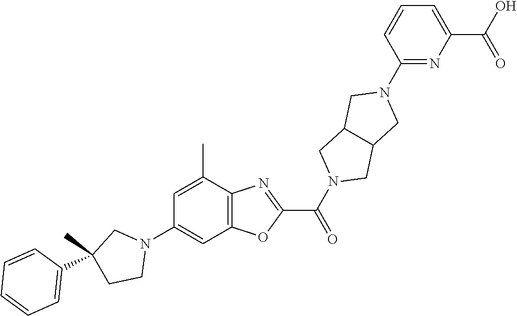 [SMILES: Cc1cc(N2CC[C@](C)(c3ccccc3)C2)cc2oc(C(=O)N3CC4CN(c5cccc(C(=O)O)n5)CC4C3)nc12] |
| 38 | 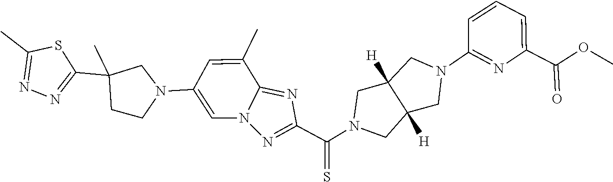 |
| 430 | 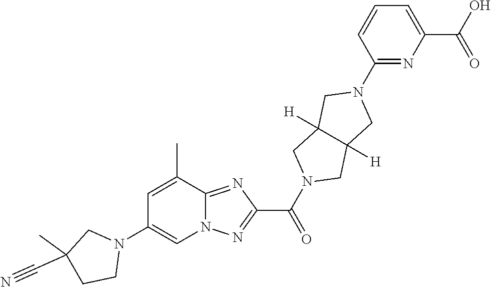 [SMILES: Cc1cc(N2CCC(C)(C#N)C2)cn2nc(C(=O)N3CC4CN(c5cccc(C(=O)O)n5)CC4C3)nc12] |
| 386 | 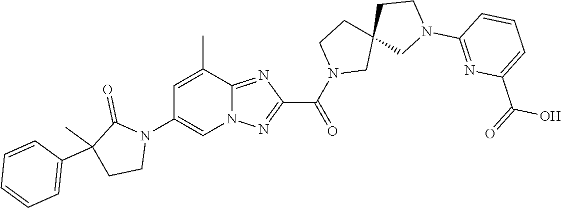 [SMILES: Cc1cc(N2CCC(C)(c3ccccc3)C2=O)cn2nc(C(=O)N3CC[C@@]4(CCN(c5cccc(C(=O)O)n5)C4)C3)nc12] |
| 387 | 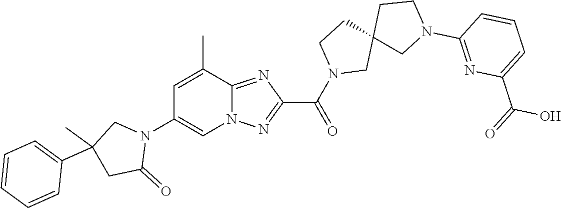 [SMILES: Cc1cc(N2CC(C)(c3ccccc3)CC2=O)cn2nc(C(=O)N3CC[C@@]4(CCN(c5cccc(C(=O)O)n5)C4)C3)nc12] |
| 25 | 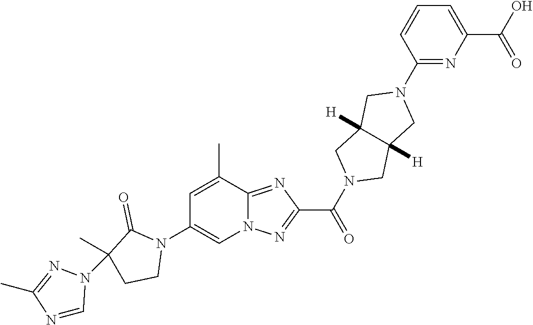 [SMILES: Cc1ncn(C2(C)CCN(c3cc(C)c4nc(C(=O)N5C[C@@H]6CN(c7cccc(C(=O)O)n7)C[C@@H]6C5)nn4c3)C2=O)n1] |
| 295 | 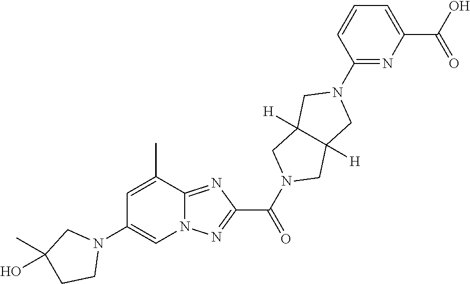 [SMILES: Cc1cc(N2CCC(C)(O)C2)cn2nc(C(=O)N3CC4CN(c5cccc(C(=O)O)n5)CC4C3)nc12] |
| 559 | 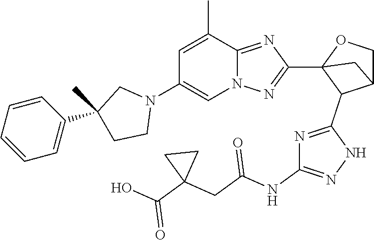 [SMILES: Cc1cc(N2CC[C@](C)(c3ccccc3)C2)cn2nc(C34CC(CO3)C4c3nc(NC(=O)CC4(C(=O)O)CC4)n[nH]3)nc12] |
| 696 | 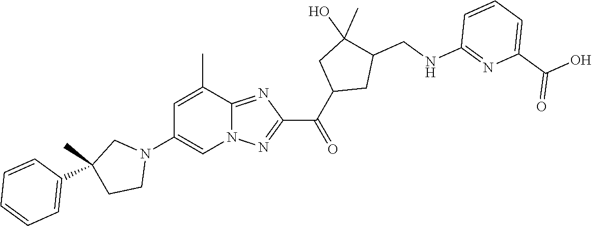 [SMILES: Cc1cc(N2CC[C@](C)(c3ccccc3)C2)cn2nc(C(=O)C3CC(CNc4cccc(C(=O)O)n4)C(C)(O)C3)nc12] |
| 728 | 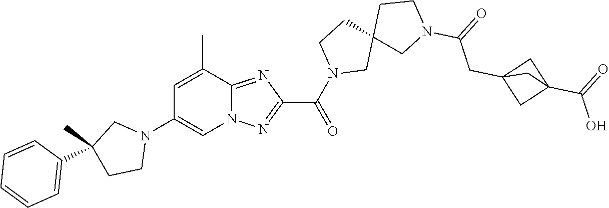 [SMILES: Cc1cc(N2CC[C@](C)(c3ccccc3)C2)cn2nc(C(=O)N3CC[C@@]4(CCN(C(=O)CC56CC(C(=O)O)(C5)C6)C4)C3)nc12] |
| 743 | 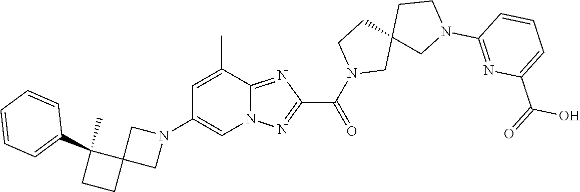 [SMILES: Cc1cc(N2CC3(CC[C@@]3(C)c3ccccc3)C2)cn2nc(C(=O)N3CC[C@@]4(CCN(c5cccc(C(=O)O)n5)C4)C3)nc12] |
| 759 | 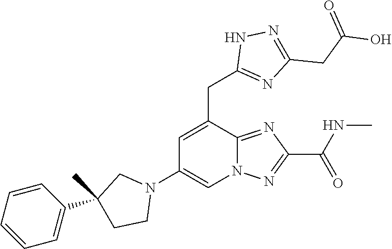 [SMILES: CNC(=O)c1nc2c(Cc3nc(CC(=O)O)n[nH]3)cc(N3CC[C@](C)(c4ccccc4)C3)cn2n1] |
| 297 | 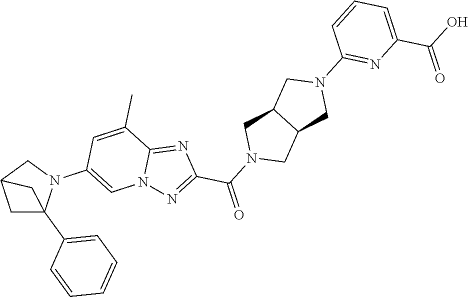 [SMILES: Cc1cc(N2CC3CC2(c2ccccc2)C3)cn2nc(C(=O)N3C[C@@H]4CN(c5cccc(C(=O)O)n5)C[C@@H]4C3)nc12] |
| 96 | 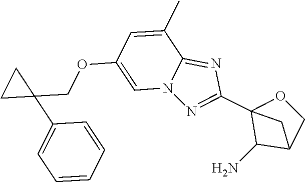 [SMILES: Cc1cc(OCC2(c3ccccc3)CC2)cn2nc(C34CC(CO3)C4N)nc12] |
| 686 | 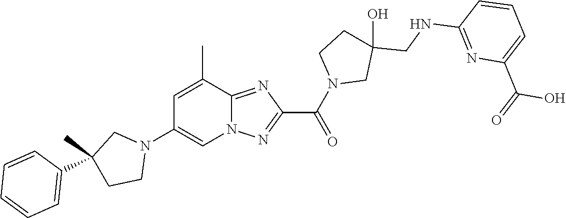 [SMILES: Cc1cc(N2CC[C@](C)(c3ccccc3)C2)cn2nc(C(=O)N3CCC(O)(CNc4cccc(C(=O)O)n4)C3)nc12] |

### Additional Exemplary Embodiments of the Compounds

In some aspects, the present disclosure provides a compound for use in the compositions and methods provided herein having Formula I*:

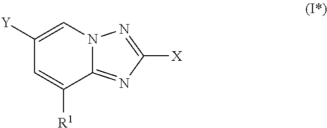

or a pharmaceutically acceptable derivative thereof, wherein:
R¹ is optionally substituted alkyl;
X is bridged bicycloheterocyclyl; and
Y is heterocyclyl or —OR², wherein R² is aralkyl.

In some embodiments, R¹ is alkyl optionally substituted with OH. In some embodiments, R¹ is —CH₂OH. In some embodiments, R¹ is unsubstituted alkyl. In some embodiments, R¹ is methyl.

In some embodiments, R² is —CH₂CR³R⁴Ph, wherein R³ and R⁴ are each independently H or methyl, or R³ and R⁴ together with the carbon atom to which they are attached form cyclopropyl. In some embodiments, R² is —CH₂CH(Me)Ph. In some embodiments, R² is

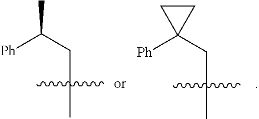

In some embodiments, X is an oxabicyclohexane, an oxabicycloheptane, an oxabicyclooctane or an oxazabicyclooctane.

In some embodiments, X is

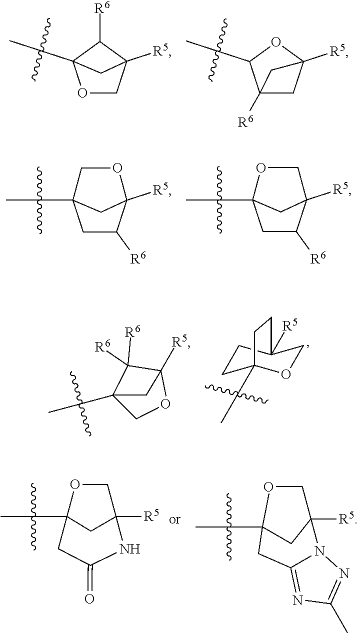

wherein R⁵ is H, optionally substituted alkyl, heterocyclyl, cycloalkyl, CN, OR⁷, NR⁸R⁹, C(O)NR¹⁰R¹¹, COOR¹² or —NR¹³C(O)R¹⁴; and each R⁶ is independently H, optionally substituted alkyl, heterocyclyl, cycloalkyl, heteroaryl, NR⁸R⁹ or C(O)NR¹⁰R¹¹;
wherein R⁷ is H or alkyl; R⁸ and R⁹ are each independently H, alkyl, —CH₂-heteroaryl or —CH₂-heterocyclyl; R¹⁰ and R¹¹ are each independently H or alkyl; R¹² is H or alkyl;
R¹³ is H or alkyl; and R¹⁴ is optionally substituted alkyl.

In some embodiments, R⁵ is H; alkyl optionally substituted with OH, NH₂ or heterocyclyl; heterocyclyl; CN; OR⁷, NR⁸R⁹; C(O)NR¹⁰R¹¹; COOR¹² or —NR¹³C(O)R¹⁴. In some embodiments, R⁵ is H; alkyl optionally substituted with OH, NH₂ or piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂₀H or —NHC(O)Me. In some embodiments, R⁵ is H; CH₂OH, CH₂NH₂, CH₂ piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂₀H or —NHC(O)Me.

In some embodiments, each R⁶ is independently H; alkyl optionally substituted with OH, NR⁸R⁹, heteroaryl, C(O)NR¹⁰R¹¹ or —NHC(O)R¹⁴; heterocyclyl; heteroaryl; NR⁸R⁹ or C(O)NR¹⁰R¹¹. In some embodiments, each R⁶ is independently H; alkyl optionally substituted with OH, NH₂, heteroaryl, C(O)NH₂ or —NHC(O)Et; heterocyclyl; heteroaryl; NR⁸R⁹ or C(O)NR¹⁰R¹¹. In some embodiments, each R⁶ is independently H; CH₂OH; C(Me)₂₀H; CH₂NH₂; CH₂C(O)NH₂; CH₂NHC(O)Et; triazolyl; oxadiazolyl; imidazolidinyl; imidazolyl; tetrazolyl; oxadiazalonyl; dimethylhydantoinyl; C(O)NH₂; C(O)NHMe; C(O)NHCH₂C(Me)₂₀H; —NHC(O)Et or NH₂.

In some embodiments, R⁷ is H or alkyl. In some embodiments, R⁷ is H or methyl. In some embodiments, R⁷ is H.

In some embodiments, R⁸ and R⁹ are each independently H, alkyl, —CH₂-triazolyl or —CH₂-heterocyclyl. In some embodiments, R⁸ and R⁹ are each independently H, methyl or —CH₂-triazolyl.

In some embodiments, R¹⁰ and R¹¹ are each independently H, methyl or ethyl.

In some embodiments, R¹² is H or methyl. In some embodiments, R¹² is H.

In some embodiments, R¹³ is H or methyl. In some embodiments, R¹³ is H.

In some embodiments, R¹⁴ is alkyl optionally substituted with OH or heteroaryl. In some embodiments, R¹⁴ is alkyl optionally substituted with OH or triazolyl. In some embodiments, R¹⁴ is methyl, —CH₂C(Me)₂₀H or CH₂-triazolyl.

In some embodiments, Y is heterocyclyl. In some embodiments, Y is pyrrolidinyl. In some embodiments, Y is

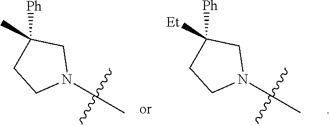 [SMILES: *N1CC[C@](C)(c2ccccc2)C1 ; *N1CC[C@](CC)(c2ccccc2)C1]

In some embodiments, Y is

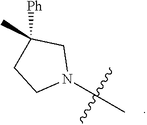 [SMILES: *N1CC[C@](C)(c2ccccc2)C1]

In some embodiments, Y is —OR². In some embodiments, Y is —O—CH₂CR³R⁴Ph, wherein R³ and R⁴ are each independently H or methyl, or R³ and R⁴ together with the carbon atom to which they are attached form cyclopropyl. In some embodiments, Y is —O—CH₂CH(Me)Ph. In some embodiments, Y is

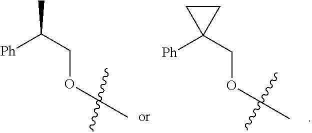 [SMILES: *OC[C@H](C)c1ccccc1 ; *OCC1(c2ccccc2)CC1]

In some embodiments, the compound for use in the compositions and methods provided herein has Formula Ia*:

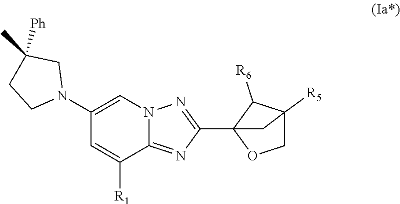

wherein R¹, R⁵ and R⁶ are as defined elsewherein herein.

In some embodiments, the compounds for use in the compositions and methods provided herein have Formula II*:

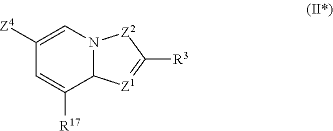

wherein R¹⁷ is OR¹⁸, NR¹⁹R²⁰, optionally substituted alkyl or CN;
Z¹ is N, CH, C—CH₂OH or C—CH₂OMe;
Z² is N or CH;
Z³ is C(O)NR²¹R²², SO₂NR²¹R²², —C(Me)₂OR²¹ or CO₂R²¹; and
Z⁴ is heterocyclyl or OR²³;
wherein R¹⁸ is H or alkyl; R¹⁹ and R^{2c} are each independently H or alkyl; R²¹ and R²² are each independently H, optionally substituted alkyl, optionally substituted cycloalkyl, bicycloalkyl or spirocycloalkyl, optionally substituted heterocyclyl, bicycloheterocyclyl or spirocycloheterocyclyl, aryl or heteroaryl, or together with the nitrogen atom to which they are attached form optionally substituted heterocyclyl, bicycloheterocyclyl or spirocycloheterocyclyl; and R²³ is aralkyl.

In some embodiments, R¹⁷ is OR¹⁸, NR¹⁹R²⁰, CN or alkyl optionally substituted with OH, NH₂, NHC(O)Me or NHSO₂Et. In some embodiments, R¹⁷ is OH, OMe, NH₂, CN, methyl, ethyl, isopropyl, CH₂OH, CH₂NH₂, CH₂NHC(O)Me or CH₂NHSO₂Et. In some embodiments, R¹⁷ is methyl.

In some embodiments, R¹⁸ is H or methyl. In some embodiments, R¹⁹ and R^{2c} are H or methyl. In some embodiments, R¹⁹ and R^{2c} are H.

In some embodiments, R²¹ and R²² are each independently H; alkyl optionally substituted with OR²⁴, heterocyclyl, bicycloheterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, COR²⁵, heterocyclyl or heteroaryl; aryl; heteroaryl; or together with the nitrogen atom to which they are attached form heterocyclyl, bicycloheterocyclyl or spirocycloheterocyclyl each optionally substituted with OR²⁴, CO₂R²⁵, heterocyclyl or heteroaryl; wherein R²⁴ and R²⁵ are each independently H or alkyl.

In some embodiments, R²¹ and R²² are each independently H; alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, C(O)Et, heterocyclyl or heteroaryl; or pyridyl; or together with the nitrogen atom to which they are attached form morpholinyl, pyrrolidinyl, piperidinyl, piperazinyl, azetidinyl, bicycloazahexyl, bicyclodiazaoctyl or spirocyclodiazanonyl each optionally substituted with OH, methoxy, CO₂H, heterocyclyl or heteroaryl.

In some embodiments, R²¹ is H or alkyl. In some embodiments, R²¹ is H. In some embodiments, R²¹ is methyl.

In some embodiments, R²² is alkyl optionally substituted with OR²⁴, heterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, heterocyclyl or heteroaryl; aryl; or heteroaryl. In some embodiments, R²² is alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, COR²⁵, heterocyclyl or heteroaryl; or pyridyl.

In some embodiments, R²¹ and R²² together with the nitrogen atom to which they are attached form heterocyclyl, bicycloheterocyclyl or spirocycloheterocyclyl each optionally substituted with OR²⁴, CO₂R²⁵, heterocyclyl or heteroaryl. In some embodiments, R²¹ and R²² together with the nitrogen atom to which they are attached form morpholinyl, pyrrolidinyl, piperidinyl, piperazinyl, azetidinyl, bicycloazahexyl, bicyclodiazaoctyl or spirocyclodiazanonyl, each optionally substituted with OH, methoxy, CO₂H, heterocyclyl or heteroaryl.

In some embodiments, R²¹ and R²² together with the nitrogen atom to which they are attached form

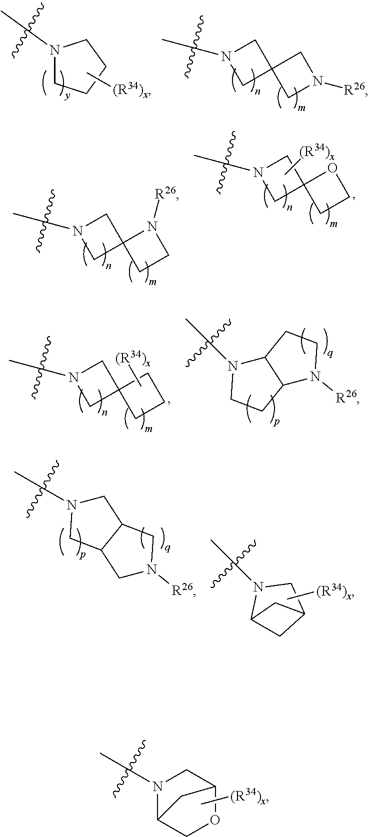

wherein x is an integer from 0-4; y is 0, 1, or 2; n is 1, 2 or 3; m is 1, 2 or 3; p is 1, 2 or 3; q is 1, 2 or 3; R²⁶ is heteroaryl optionally substituted with CO₂R²⁷ wherein R²⁷ is H or alkyl; and R³⁴ is OR²⁴, CO₂R²⁵, heterocyclyl or heteroaryl.

In some embodiments, x is 0. In some embodiments, x is 1. In some embodiments, x is 2. In some embodiments, x is 3. In some embodiments, y is 0. In some embodiments, y is 1. In some embodiments, y is 2. In some embodiments, n is 1. In some embodiments, n is 2. In some embodiments, n is 3. In some embodiments, m is 1. In some embodiments, m is 2. In some embodiments, m is 3. In some embodiments, p is 1. In some embodiments, p is 2. In some embodiments, p is 3. In some embodiments, q is 1. In some embodiments, q is 2. In some embodiments, q is 3.

In some embodiments, R²⁶ is pyridyl optionally substituted with CO₂R²⁷, wherein R²⁷ is H or alkyl. In some embodiments, R²⁷ is H. In some embodiments, R³⁴ is OH, methoxy, CO₂H, heterocyclyl or heteroaryl.

In some embodiments, Z⁴ is OR²³. In some embodiments, R²³ is —CH₂CR²⁸R²⁹Ph, wherein R²⁸ and R²⁹ are each independently H or methyl, or R²⁸ and R²⁹ together with the carbon atom to which they are attached form cyclopropyl. In some embodiments, R²³ is —CH₂CH(Me)Ph. In some embodiments, R²³ is

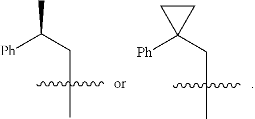

In some embodiments, Z¹ is N or CH. In some embodiments, Z¹ is N. In some embodiments, Z¹ is CH.

In some embodiments, Z² is N. In some embodiments, Z² is CH.

In some embodiments, Z¹ is N and Z² is CH. In some embodiments, Z¹ is CH and Z² is N. In some embodiments, Z¹ and Z² are both N.

In some embodiments, Z³ is C(O)NR²¹R²², SO₂NR²¹R²² or CO₂R²¹. In some embodiments, Z³ is C(O)NR²¹R²² or SO₂NR²¹R²². In some embodiments, Z³ is C(O)NR²¹R²².

In some embodiments, Z⁴ is heterocyclyl. In some embodiments, Z⁴ is pyrrolidinyl, azetidinyl, morpholinyl or azaspiroheptyl. In some embodiments, Z⁴ is pyrrolidinyl, azetidinyl, morpholinyl or azaspiro[3.3.0]heptyl. In some embodiments, Z⁴ is pyrrolidinyl, azetidinyl, morpholinyl or azaspiro[3.3.0]heptyl, each optionally substituted with CN, OH, optionally substituted alkyl, aryl or heteroaryl. In some embodiments, Z⁴ is pyrrolidinyl, azetidinyl, morpholinyl or azaspiro[3.3.0]heptyl, each optionally substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl. In some embodiments, Z⁴ is azaspiro[3.3.0]heptyl substituted with phenyl. In some embodiments, Z⁴ is morpholinyl substituted with phenyl and methyl. In some embodiments, Z⁴ is azetidinyl substituted with (i) methyl or ethyl, and (ii) phenyl, 3-methylphenyl or 4-methylphenyl.

In some embodiments, Z⁴ is pyrrolidinyl substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl.

In some embodiments, Z⁴ is

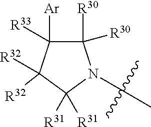

wherein each R³⁰ is H or the two R³⁰ form oxo;
each R³¹ is H or the two R³¹ form oxo;
each R³² is H or methyl;
R³³ is optionally substituted alkyl, or together with R³¹ forms a bridged bicyclic pyrrolidinyl group; and
Ar is an optionally substituted aryl or heteroaryl group, OH or CN.

In some embodiments, each R³⁰, R³¹ and R³² is H.

In some embodiments, R³³ is optionally substituted alkyl. In some embodiments, R³³ is optionally substituted methyl. In some embodiments, R³³ is methyl.

In some embodiments, Ar is aryl or heteroaryl optionally substituted with methyl, chloro, fluoro, methoxy or hydroxy. In some embodiments, Ar is phenyl, pyridyl, thiazolyl, oxazolyl, pyrazolyl, oxadiazolyl, tetrazolyl, thiadiazolyl or triazolyl, each optionally substituted with methyl, chloro, fluoro, methoxy or hydroxy. In some embodiments, Ar is phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl. In some embodiments, Ar is phenyl optionally substituted with methyl, chloro, fluoro, methoxy or hydroxy. In some embodiments, Ar is unsubstituted phenyl.

In some embodiments, Z⁴ is

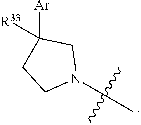

In some embodiments, Z⁴ is

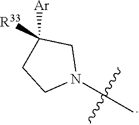

In some embodiments, Z⁴ is

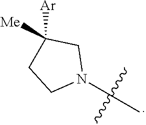

In some embodiments, Z⁴ is

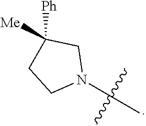 [SMILES: *N1CC[C@](C)(c2ccccc2)C1]

In some embodiments, the compound for use in the compositions and methods provided herein has one of the following formulae:

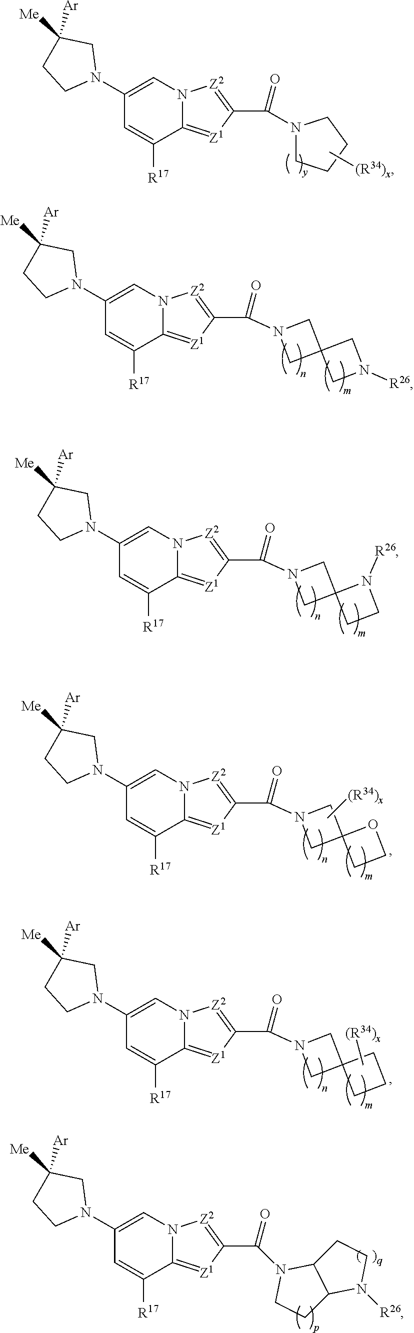

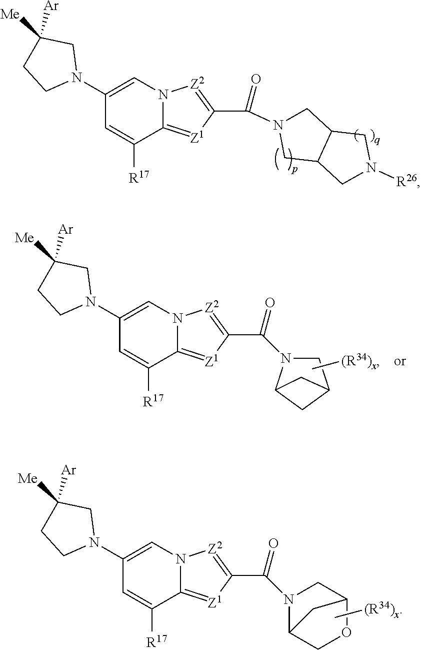

In some embodiments, the compound for use in the compositions and methods the present disclosure provides selected from the compounds provided in the Examples.

### Synthesis of the Compounds

The compound provided herein may be synthesized using methods well known to those of skill in the art. For example, the compounds may be prepared according to Scheme I:

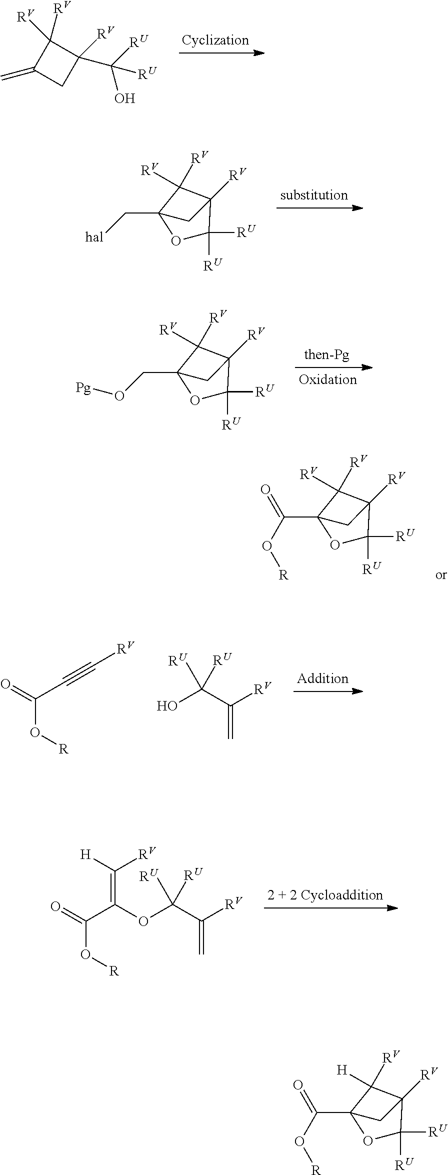

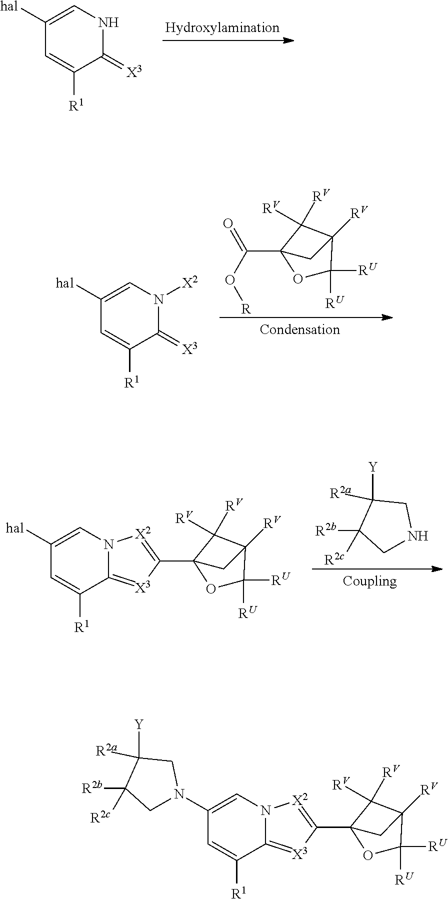

wherein hal is a halogen and Pg is a protecting group.

In some embodiments, the compounds provided herein may be prepared according to Scheme II:

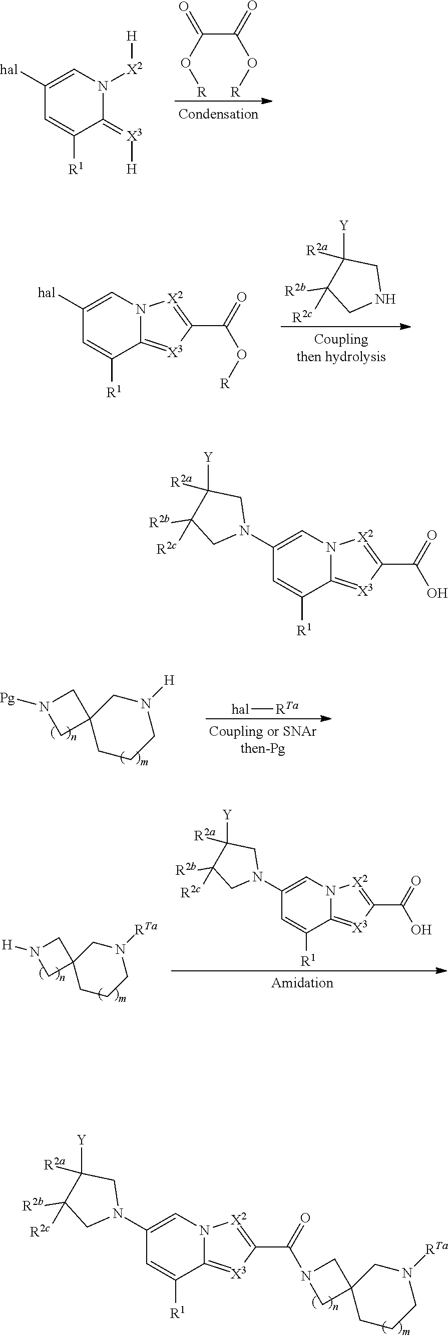

### Pharmaceutical Compositions

In some aspects, the present disclosure features pharmaceutical compositions comprising a compound described herein, and one or more pharmaceutically acceptable carriers or excipients.

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In some embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In some embodiments, a therapeutically effective dosage should produce a serum concentration of active ingredient of from about 0.1 ng/mL to about 50-100 μg/mL. In some embodiments, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in some embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in some embodiments, about 0.1-85% active ingredient, or, in other embodiments, about 75-95% active ingredient.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In some embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof, and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound the present disclosure provides dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Some embodiments is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in some embodiments, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In some embodiments, the use of an optimally designed controlled-release preparation in medical treatment is characterized by a minimum of drug substance being employed to cure or control the condition in a minimum amount of time. In some embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In some embodiments, the agent may be administered using intravenous infusion, an implantable osmotic pump, a transdermal patch, liposomes, or other modes of administration. In some embodiments, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In some embodiments, polymeric materials can be used. In yet some embodiments, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In some embodiments, provided herein also are kits which, when used by the medical practitioner, can simplify the administration of appropriate amounts of active ingredients to a subject. In some embodiments, the kit provided herein includes a container and a dosage form of a compound provided herein, including a single enantiomer or a mixture of diastereomers thereof; or a pharmaceutically acceptable salt, solvate, or prodrug thereof.

In some embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### Dosings

The compounds and pharmaceutical compositions provided herein may be dosed in certain therapeutically or prophylactically effective amounts, certain time intervals, certain dosage forms, and certain dosage administration methods as described below.

In some embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In some embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In some embodiments, the recommended daily dose range of the compound provided herein, or a derivative thereof, for the conditions described herein lie within the range of from about 0.5 mg to about 50 mg per day, in some embodiments given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In some embodiments, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In some embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In some embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In some embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In some embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In some embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In some embodiments, the compound provided herein, or a derivative thereof, is administered orally. In some embodiments, the compound provided herein, or a derivative thereof, is administered parenterally. In yet some embodiments, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In some embodiments, administration is once a day, twice a day, three times a day, four times a day, once every other day, twice a week, once every week, once every two weeks, once every three weeks, or once every four weeks. In some embodiments, the compound provided herein, or a derivative thereof, is administered once a day. In some embodiments, the compound provided herein, or a derivative thereof, is administered twice a day. In yet some embodiments, the compound provided herein, or a derivative thereof, is administered three times a day. In still some embodiments, the compound provided herein, or a derivative thereof, is administered four times a day.

In some embodiments, the compound provided herein, or a derivative thereof, is administered once per day from one day to six months, from one week to three months, from one week to four weeks, from one week to three weeks, or from one week to two weeks. In some embodiments, the compound provided herein, or a derivative thereof, is administered once per day for one week, two weeks, three weeks, or four weeks. In some embodiments, the compound provided herein, or a derivative thereof, is administered once per day for 4 days. In some embodiments, the compound provided herein, or a derivative thereof, is administered once per day for 5 days. In some embodiments, the compound provided herein, or a derivative thereof, is administered once per day for 6 days. In some embodiments, the compound provided herein, or a derivative thereof, is administered once per day for one week. In some embodiments, the compound provided herein, or a derivative thereof, is administered once per day for two weeks. In yet some embodiments, the compound provided herein, or a derivative thereof, is administered once per day for three weeks. In still some embodiments, the compound provided herein, or a derivative thereof, is administered once per day for four weeks.

### Methods of Use

In some aspects, the present disclosure provides a method of treating or preventing a disease (e.g., light chain amyloidosis) in a subject, comprising administering to the subject a compound or composition provided herein (e.g., in a therapeutically effective amount).

In some aspects, the present disclosure provides a compound or composition provided herein, for treating or preventing a disease (e.g., light chain amyloidosis) in a subject.

In some aspects, the present disclosure provides use of a compound provided herein in the manufacture of a medicament for treating or preventing a disease (e.g., light chain amyloidosis) in a subject.

In some embodiments, the disease is light chain amyloidosis.

In some aspects, the present disclosure provides a method of treating or preventing light chain amyloidosis in a subject, comprising administering to the subject a compound or composition provided herein (e.g., in a therapeutically effective amount).

In some aspects, the present disclosure provides a compound or composition provided herein, for treating or preventing light chain amyloidosis in a subject.

In some aspects, the present disclosure provides use of a compound provided herein in the manufacture of a medicament for treating or preventing light chain amyloidosis in a subject

In some aspects, the present disclosure provides a method of stabilizing immunoglobulin light chains in a subject, comprising administering to the subject a compound or composition provided herein (e.g., in a therapeutically effective amount).

In some aspects, the present disclosure provides a compound or composition provided herein, for stabilizing immunoglobulin light chains in a subject.

In some aspects, the present disclosure provides use of a compound provided herein in the manufacture of a medicament for stabilizing immunoglobulin light chains in a subject.

In some aspects, the present disclosure provides a method of preventing or lessening immunoglobulin light chain misfolding and/or endoproteolysis in a subject, comprising administering to the subject a compound or composition provided herein (e.g., in a therapeutically effective amount).

In some aspects, the present disclosure provides a compound or composition provided herein, for preventing or lessening immunoglobulin light chain misfolding and/or endoproteolysis in a subject.

In some aspects, the present disclosure provides use of a compound provided herein in the manufacture of a medicament for preventing or lessening immunoglobulin light chain misfolding and/or endoproteolysis in a subject.

In some embodiments, the immunoglobulin light chains are stabilized in a native conformation thereof. In some embodiments, the immunoglobulin light chains are dimers.

In some aspects, the present disclosure provides a method of maintenance therapy upon recurrence of light chain amyloidosis following primary treatment by administering to a subject a compound or composition provided herein.

In some aspects, the present disclosure provides a compound or composition provided herein, for maintenance therapy upon recurrence of light chain amyloidosis following primary treatment.

In some aspects, the present disclosure provides use of a compound provided herein in the manufacture of a medicament for maintenance therapy upon recurrence of light chain amyloidosis following primary treatment.

In some aspects, the present disclosure provides a method of combination therapy using a compound or composition provided herein in combination with one or more additional active agents that treat light chain amyloidosis, deplete clonal plasma cells, stabilize immunoglobulin light chains, prevent or lessen immunoglobulin light chain misfolding and/or endoproteolysis, promote clearance of fibrils, or that are effective in maintenance therapy upon recurrence of light chain amyloidosis following primary treatment.

Free light chains (FLCs) adopt a well-defined homodimeric structure, wherein the monomers may be covalently linked by an interchain disulfide bond. LC monomers comprise an N-terminal variable (V) domain attached to a C-terminal constant (C) domain. Amyloidogenic FLCs involved in AL patients, are less stable than non-amyloidogenic FLCs, can misfold and misassemble into nonnative species including cross-β-sheet amyloid fibrils, a hallmark of AL amyloidosis. The structure-proteotoxicity relationship is not fully understood but several processes have been described, including destabilization-dependent endoproteolysis that releases amyloidogenic LC fragments. LC fragments including V domains are observed in patient deposits alongside full length LCs. Without being bound by any theory, it is believed that the compounds and compositions provided herein stop light chain conformational excursions at the beginning of the aggregation cascade via kinetic stabilization of FLCs. It is also believed that the compounds provided herein bind to conserved regions of LCs, thereby allowing the compounds to stabilize many immunoglobulin light chains and to be effective in many AL subjects.

Kinetic stabilization of LCs is unlikely to contribute to plasma cell death but could reduce organ proteotoxicity and the progression of AL. Subjects with prominent cardiac involvement currently have few available options for treatment and represent an urgent unmet medical need, as they are often too sick to tolerate chemotherapy. Reduction of organ proteotoxicity could allow the subject to tolerate chemotherapy. Thus, in some embodiments, the present disclosure provides a method of pretreatment of a subject having AL with prominent cardiac involvement with a compound provided herein, followed by chemotherapy.

In some embodiments, the present disclosure provides a method of treating light chain amyloidosis by administering to a subject a compound or composition provided herein. In some embodiments, the subject has light chain amyloidosis and is treatment naive. In some embodiments, the subject has relapsed or refractory light chain amyloidosis.

In some embodiments, the present disclosure provides a method of stabilizing immunoglobulin light chains by contacting the immunoglobulin light chains with a compound provided herein. In some embodiments, the immunoglobulin light chains are stabilized in a native conformation thereof. In some embodiments, the immunoglobulin light chains are dimers. Thus, in some embodiments, the present disclosure provides a method of stabilizing immunoglobulin light chain dimers in a native conformation. As used herein, “native conformation” refers to a conformation of immunoglobulin light chains present in subjects not having light chain amyloidosis.

In some embodiments, the present disclosure provides a method of preventing or lessening immunoglobulin light chain misfolding and/or endoproteolysis by contacting the immunoglobulin light chains with a compound provided herein.

In some embodiments, provided is a method of maintenance therapy upon recurrence of light chain amyloidosis following primary treatment by administering to a subject a compound or composition provided herein. In such embodiments, reemergence of the clonal plasma cells is generally slow, and thus organ toxicity caused by conformationally unstable circulating LC can be minimized by kinetic stabilizer treatment.

### Combinations

The compound provided herein, or a derivative thereof, can also be combined or used in combination with other therapeutic agents useful in the treatment and/or prevention of light chain amyloidosis.

In some embodiments, the present disclosure provides a method of treating, preventing, or managing light chain amyloidosis, comprising administering to a subject a compound provided herein, or a derivative thereof; in combination with one or more second active agents. In some embodiments, the second active agent is a proteasome inhibitor (e.g., bortezomib, ixazomib, carfilzomib). In some embodiments, the second active agent is a chemotherapeutic agent, including but not limited to alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129 (sorrentotherapeutics.com)) and/or an anti-amyloid antibody. In some embodiments, the compounds provided herein, or a derivative thereof, are used in combination with stem cell transplant therapy. In further embodiments, the second active agent is selected from those disclosed in PCT Publication Nos. WO 2020/205683, WO 2019/191558, WO 2017/117430 or WO 2021/007594. In some embodiments, the second active agent is a therapeutic agent that promotes clearance of amyloid deposits, such as CAEL-101 (caelumbio.com) or NEOD001 (birtamimab).

In some embodiments, the second active agent is a plasma cell directed therapy, including high-dose cyclophosphamide combined with an anti-thymocyte antibody (e.g., Thymoglobulin®, Atgam®)(with subsequent autologous stem cell transplantation), an immunomodulatory agent (e.g., lenalidomide), a steroid (e.g., dexamethasone), a proteasome inhibitor (e.g., bortezomib), atacicept, or an anti-CD38 antibody (e.g., daratumumab, isatuximab). In some embodiments, the second active agent is a combination of daratumumab, bortezomib, cyclophosphamide and dexamethasone.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In some embodiments, the compound provided herein, or a derivative thereof, is administered orally. In some embodiments, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In some embodiments, the compound provided herein, or a derivative thereof, and a second therapy are administered by the same mode of administration, orally or by IV. In some embodiments, the compound provided herein, or a derivative thereof, is administered by one mode of administration, e.g., by IV, whereas the second agent is administered by another mode of administration, e.g., orally.

In some embodiments, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

In some embodiments, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein, the term “subject” refers to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

In some embodiments, the term “subject in need thereof” can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject in need thereof may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound provided herein can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) wherein R is alkyl, alkenyl, alkynyl, aryl, aralkyl and cycloalkyl. Pharmaceutically acceptable enol esters include, but are not limited to, derivatives of formula C═C(OC(O)R) wherein R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating AL amyloidosis.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

Wherein moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′— and —R′C(O)₂—.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocyclyl” refers to a saturated or partially unsaturated 3-8 membered monocyclic, 6-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocyclyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocyclyl, only one of the rings in the heterocyclyl needs to be non-aromatic.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in some embodiments from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in some embodiments, selected from, deuterium, —OR′, ═O, ═NR′, ═N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NRSO₂NR′R″, —CN and —NO₂ in a number ranging from zero to the number of hydrogen atoms in such radical. In some embodiments, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in some embodiments independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in some embodiments, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and wherein R′, R″, R′″ and R″″ are, in some embodiments, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)r-B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)_{d}—, wherein s and d are independently integers of from 0 to 3, and X′ is —O—, —NR′—, —S—, —S(O)—, —S(O)₂—, or —S(O)₂NR′—. The substituent moieties R, R′, R″ and R′″ are, in some embodiments, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Numerated Exemplary Embodiments

Exemplary Embodiment No. 1. A compound of Formula I*:

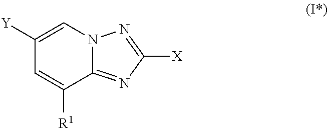

or a pharmaceutically acceptable derivative thereof, wherein:
   R¹ is optionally substituted alkyl;
   X is bridged bicycloheterocyclyl; and
   Y is heterocyclyl or —OR², wherein R² is aralkyl.
Exemplary Embodiment No. 2. The compound of Exemplary Embodiment No. 1, wherein R¹ is alkyl optionally substituted with OH.
Exemplary Embodiment No. 3. The compound of Exemplary Embodiment No. 1 or Exemplary Embodiment No. 2, wherein R¹ is —CH₂OH.
Exemplary Embodiment No. 4. The compound of Exemplary Embodiment No. 1, wherein R¹ is unsubstituted alkyl.
Exemplary Embodiment No. 5. The compound of Exemplary Embodiment No. 1 or Exemplary Embodiment No. 4, wherein R¹ is methyl.
Exemplary Embodiment No. 6. The compound of any one of Exemplary Embodiment Nos. 1-5, wherein R² is —CH₂CR³R⁴Ph, wherein R³ and R⁴ are each independently H or methyl, or R³ and R⁴ together with the carbon atom to which they are attached form cyclopropyl.
Exemplary Embodiment No. 7. The compound of any one of Exemplary Embodiment Nos. 1-6, wherein R² is —CH₂CH(Me)Ph.
Exemplary Embodiment No. 8. The compound of any one of Exemplary Embodiment Nos. 1-6 wherein R² is

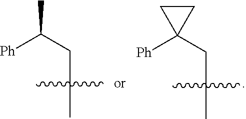

Exemplary Embodiment No. 9. The compound of any one of Exemplary Embodiment Nos. 1-8, wherein X is an oxabicyclohexane, an oxabicycloheptane, an oxabicyclooctane or an oxazabicyclooctane.
Exemplary Embodiment No. 10. The compound of any one of Exemplary Embodiment Nos. 1-9, wherein X is

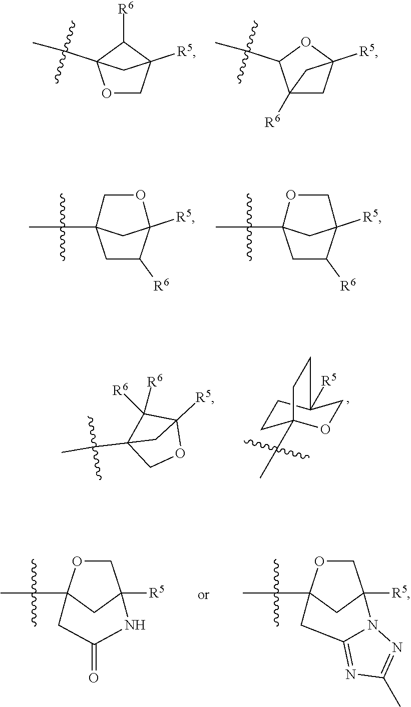

wherein R⁵ is H, optionally substituted alkyl, heterocyclyl, cycloalkyl, CN, OR⁷, NR¹R⁹, C(O)NR¹⁰R¹¹, COOR¹² or —NR¹³C(O)R¹⁴; and each R⁶ is independently H, optionally substituted alkyl, heterocyclyl, cycloalkyl, heteroaryl, NR′R⁹ or C(O)NR¹⁰R¹¹;
wherein R⁷ is H or alkyl; R¹ and R⁹ are each independently H, alkyl, —CH₂-heteroaryl or —CH₂-heterocyclyl; R¹⁰ and R″ are each independently H or alkyl; R¹² is H or alkyl; R¹³ is H or alkyl; and R¹⁴ is optionally substituted alkyl.
Exemplary Embodiment No. 11. The compound of Exemplary Embodiment No. 10, wherein R⁵ is H; alkyl optionally substituted with OH, NH₂ or heterocyclyl; heterocyclyl; CN; OR⁷, NR′R⁹; C(O)NR¹⁰R¹¹; COOR¹² or —NR¹³C(O)R¹⁴.
Exemplary Embodiment No. 12. The compound of Exemplary Embodiment No. 10, wherein R⁵ is H; alkyl optionally substituted with OH, NH₂ or piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂₀H or —NHC(O)Me.
Exemplary Embodiment No. 13. The compound of Exemplary Embodiment No. 10, wherein R⁵ is H; CH₂OH, CH₂NH₂, CH₂ piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂₀H or —NHC(O)Me.
Exemplary Embodiment No. 14. The compound of Exemplary Embodiment No. 10, wherein each R⁶ is independently H; alkyl optionally substituted with OH, NR′R⁹, heteroaryl, C(O)NR¹⁰R¹¹ or —NHC(O)R¹⁴; heterocyclyl; heteroaryl; NR′R⁹ or C(O)NR¹⁰R¹¹.
Exemplary Embodiment No. 15. The compound of Exemplary Embodiment No. 10, wherein R⁶ is independently H; alkyl optionally substituted with OH, NH₂, heteroaryl, C(O)NH₂ or —NHC(O)Et; heterocyclyl; heteroaryl; NR⁸R⁹ or C(O)NR¹⁰R¹¹.
Exemplary Embodiment No. 16. The compound of Exemplary Embodiment No. 10, wherein each R⁶ is independently H; CH₂OH; C(Me)₂₀H; CH₂NH₂; CH₂C(O)NH₂; CH₂NHC(O)Et; triazolyl; oxadiazolyl; imidazolidinyl; imidazolyl; tetrazolyl; oxadiazalonyl; dimethylhydantoinyl; C(O)NH₂; C(O)NHMe; C(O)NHCH₂C(Me)₂₀H; —NHC(O)Et or NH₂.
Exemplary Embodiment No. 17. The compound of any one of Exemplary Embodiment Nos. 10-16, wherein R⁷ is H or alkyl.
Exemplary Embodiment No. 18. The compound of any one of Exemplary Embodiment Nos. 10-17, wherein R⁷ is H or methyl.
Exemplary Embodiment No. 19. The compound of any one of Exemplary Embodiment Nos. 10-18, wherein R⁷ is H.
Exemplary Embodiment No. 20. The compound of any one of Exemplary Embodiment Nos. 10-19, wherein R⁸ and R⁹ are each independently H, alkyl, —CH₂-triazolyl or —CH₂-heterocyclyl.
Exemplary Embodiment No. 21. The compound of any one of Exemplary Embodiment Nos. 10-20, wherein R⁸ and R⁹ are each independently H, methyl or —CH₂-triazolyl.
Exemplary Embodiment No. 22. The compound of any one of Exemplary Embodiment Nos. 10-21, wherein R¹⁰ and R″ are each independently H, methyl or ethyl.
Exemplary Embodiment No. 23. The compound of any one of Exemplary Embodiment Nos. 10-22, wherein R¹² is H or methyl.
Exemplary Embodiment No. 24. The compound of any one of Exemplary Embodiment Nos. 10-23, wherein R¹² is H.
Exemplary Embodiment No. 25. The compound of any one of Exemplary Embodiment Nos. 10-24, wherein R¹³ is H or methyl.
Exemplary Embodiment No. 26. The compound of any one of Exemplary Embodiment Nos. 10-25, wherein R¹³ is H.
Exemplary Embodiment No. 27. The compound of any one of Exemplary Embodiment Nos. 10-26, wherein R¹⁴ is alkyl optionally substituted with OH or heteroaryl.
Exemplary Embodiment No. 28. The compound of any one of Exemplary Embodiment Nos. 10-27, wherein R¹⁴ is alkyl optionally substituted with OH or triazolyl.
Exemplary Embodiment No. 29. The compound of any one of Exemplary Embodiment Nos. 10-28, wherein R¹⁴ is methyl, —CH₂C(Me)₂₀H or CH₂-triazolyl.
Exemplary Embodiment No. 30. The compound of any one of Exemplary Embodiment Nos. 1-29, wherein Y is heterocyclyl.
Exemplary Embodiment No. 31. The compound of any one of Exemplary Embodiment Nos. 1-30, wherein Y is pyrrolidinyl.
Exemplary Embodiment No. 32. The compound of any one of Exemplary Embodiment Nos. 1-31, wherein Y is

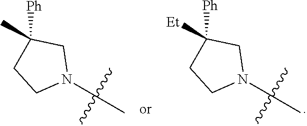 [SMILES: *N1CC[C@](C)(c2ccccc2)C1 ; *N1CC[C@](CC)(c2ccccc2)C1]

Exemplary Embodiment No. 33. The compound of any one of Exemplary Embodiment Nos. 1-32, wherein Y is

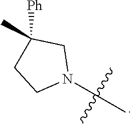 [SMILES: *N1CC[C@](C)(c2ccccc2)C1]

Exemplary Embodiment No. 34. The compound of any one of Exemplary Embodiment Nos. 1-29, wherein Y is —OR².
Exemplary Embodiment No. 35. The compound of any one of Exemplary Embodiment Nos. 1-29 and 34, wherein Y is —O—CH₂CR³R⁴Ph, wherein R³ and R⁴ are each independently H or methyl, or R³ and R⁴ together with the carbon atom to which they are attached form cyclopropyl.
Exemplary Embodiment No. 36. The compound of any one of Exemplary Embodiment Nos. 1-29, 34 and 35, wherein Y is —O—CH₂CH(Me)Ph.
Exemplary Embodiment No. 37. The compound of any one of Exemplary Embodiment Nos. 1-29, 34 and 35, wherein Y is

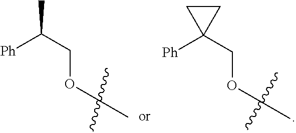 [SMILES: *OC[C@H](C)c1ccccc1 ; *OCC1(c2ccccc2)CC1]

Exemplary Embodiment No. 38. The compound of any one of Exemplary Embodiment Nos. 1-33, wherein the compound has Formula Ia*:

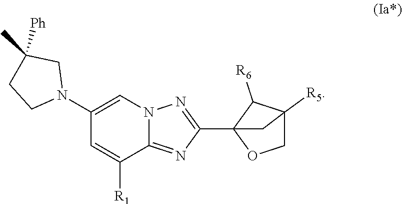

Exemplary Embodiment No. 39. A compound of Formula II*:

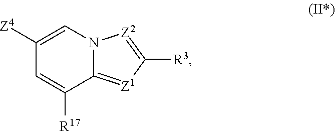

or a pharmaceutically acceptable derivative thereof, wherein:
   R¹⁷ is OR¹⁸, NR¹⁹R²⁰, optionally substituted alkyl or CN;
   Z¹ is N, CH, C—CH₂OH or C—CH₂OMe;
   Z² is N or CH;
   Z³ is C(O)NR²¹R²², SO₂NR²¹R²², —C(Me)₂OR²¹ or CO₂R²¹; and
   Z⁴ is heterocyclyl or OR²³;
   wherein R¹⁸ is H or alkyl; R¹⁹ and R^{2c} are each independently H or alkyl; R²¹ and R²² are each independently H, optionally substituted alkyl, optionally substituted cycloalkyl, bicycloalkyl or spirocycloalkyl, optionally substituted heterocyclyl, bicycloheterocyclyl or spirocycloheterocyclyl, aryl or heteroaryl, or together with the nitrogen atom to which they are attached form optionally substituted heterocyclyl, bicycloheterocyclyl or spirocycloheterocyclyl; and R²³ is aralkyl.
Exemplary Embodiment No. 40. The compound of Exemplary Embodiment No. 39, wherein R¹⁷ is OR¹⁸, NR¹⁹R²⁰, CN or alkyl optionally substituted with OH, NH₂, NHC(O)Me or NHSO₂Et.
Exemplary Embodiment No. 41. The compound of Exemplary Embodiment No. 39 or Exemplary Embodiment No. 40, wherein R¹⁷ is OH, OMe, NH₂, CN, methyl, ethyl, isopropyl, CH₂OH, CH₂NH₂, CH₂NHC(O)Me or CH₂NHSO₂Et.
Exemplary Embodiment No. 42. The compound of any one of Exemplary Embodiment Nos. 39-41, wherein R¹⁷ is methyl.
Exemplary Embodiment No. 43. The compound of Exemplary Embodiment No. 39 or Exemplary Embodiment No. 40, wherein R¹⁸ is H or methyl.
Exemplary Embodiment No. 44. The compound of Exemplary Embodiment No. 39 or Exemplary Embodiment No. 40, wherein R¹⁹ and R^{2c} are H or methyl.
Exemplary Embodiment No. 45. The compound of Exemplary Embodiment No. 39 or Exemplary Embodiment No. 40, wherein R¹⁹ and R^{2c} are H.
Exemplary Embodiment No. 46. The compound of any one of Exemplary Embodiment Nos. 39-45, wherein R²¹ and R²² are each independently H; alkyl optionally substituted with OR²⁴, heterocyclyl, bicycloheterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, COR²⁵, heterocyclyl or heteroaryl; aryl; heteroaryl; or together with the nitrogen atom to which they are attached form heterocyclyl, bicycloheterocyclyl or spirocycloheterocyclyl each optionally substituted with OR²⁴, CO₂R²⁵, heterocyclyl or heteroaryl; wherein R²⁴ and R²⁵ are each independently H or alkyl.
Exemplary Embodiment No. 47. The compound of any one of Exemplary Embodiment Nos. 39-467, wherein R²¹ and R²² are each independently H; alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, C(O)Et, heterocyclyl or heteroaryl; or pyridyl; or together with the nitrogen atom to which they are attached form morpholinyl, pyrrolidinyl, piperidinyl, piperazinyl, azetidinyl, bicycloazahexyl, bicyclodiazaoctyl or spirocyclodiazanonyl each optionally substituted with OH, methoxy, CO₂H, heterocyclyl or heteroaryl.
Exemplary Embodiment No. 48. The compound of any one of Exemplary Embodiment Nos. 39-47, wherein R²¹ is H or alkyl.
Exemplary Embodiment No. 49. The compound of any one of Exemplary Embodiment Nos. 39-48, wherein R²¹ is H.
Exemplary Embodiment No. 50. The compound of any one of Exemplary Embodiment Nos. 39-48, wherein R²¹ is methyl.
Exemplary Embodiment No. 51. The compound of any one of Exemplary Embodiment Nos. 39-50, wherein R²² is alkyl optionally substituted with OR²⁴ heterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, heterocyclyl or heteroaryl; aryl; or heteroaryl.
Exemplary Embodiment No. 52. The compound of any one of Exemplary Embodiment Nos. 39-51, wherein R²² is alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, COR²⁵, heterocyclyl or heteroaryl; or pyridyl.
Exemplary Embodiment No. 53. The compound of any one of Exemplary Embodiment Nos. 39-47, wherein R²¹ and R²² together with the nitrogen atom to which they are attached form heterocyclyl, bicycloheterocyclyl or spirocycloheterocyclyl each optionally substituted with OR²⁴, CO₂R²⁵, heterocyclyl or heteroaryl.
Exemplary Embodiment No. 54. The compound of any one of Exemplary Embodiment Nos. 39-47 and 53, wherein R²¹ and R²² together with the nitrogen atom to which they are attached form morpholinyl, pyrrolidinyl, piperidinyl, piperazinyl, azetidinyl, bicycloazahexyl, bicyclodiazaoctyl or spirocyclodiazanonyl, each optionally substituted with OH, methoxy, CO₂H, heterocyclyl or heteroaryl.
Exemplary Embodiment No. 55. The compound of any one of Exemplary Embodiment Nos. 39-47, 53 and 54, wherein R²¹ and R²² together with the nitrogen atom to which they are attached form

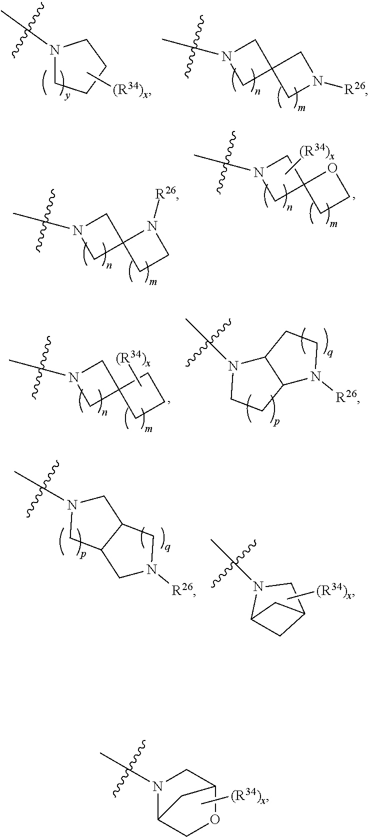

wherein x is an integer from 0-4; y is 0, 1, or 2; n is 1, 2 or 3; m is 1, 2 or 3; p is 1, 2 or 3; q is 1, 2 or 3; R²⁶ is heteroaryl optionally substituted with CO₂R²⁷ wherein R²⁷ is H or alkyl; and R³⁴ is OR²⁴, CO₂R²⁵, heterocyclyl or heteroaryl.
Exemplary Embodiment No. 56. The compound of Exemplary Embodiment No. 55, wherein R²⁶ is pyridyl optionally substituted with CO₂R²⁷, wherein R²⁷ is H or alkyl.
Exemplary Embodiment No. 57. The compound of Exemplary Embodiment No. 56, wherein R²⁷ is H.
Exemplary Embodiment No. 58. The compound of Exemplary Embodiment No. 55, wherein R³⁴ is OH, methoxy, CO₂H, heterocyclyl or heteroaryl.
Exemplary Embodiment No. 59. The compound of any one of Exemplary Embodiment Nos. 39-58, wherein Z⁴ is OR²³.
Exemplary Embodiment No. 60. The compound of any one of Exemplary Embodiment Nos. 39-59, wherein R²³ is —CH₂CR²⁸R²⁹Ph, wherein R²⁸ and R²⁹ are each independently H or methyl, or R²⁸ and R²⁹ together with the carbon atom to which they are attached form cyclopropyl.
Exemplary Embodiment No. 61. The compound of any one of Exemplary Embodiment Nos. 39-60, wherein R²³ is —CH₂CH(Me)Ph.
Exemplary Embodiment No. 62. The compound of any one of Exemplary Embodiment Nos. 39-60, wherein R²³ is

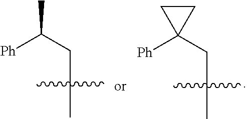

Exemplary Embodiment No. 63. The compound of any one of Exemplary Embodiment Nos. 39-62, wherein Z¹ is N or CH.
Exemplary Embodiment No. 64. The compound of any one of Exemplary Embodiment Nos. 39-63, wherein Z¹ is N.
Exemplary Embodiment No. 65. The compound of any one of Exemplary Embodiment Nos. 39-63, wherein Z¹ is CH.
Exemplary Embodiment No. 66. The compound of any one of Exemplary Embodiment Nos. 39-65, wherein Z² is N.
Exemplary Embodiment No. 67. The compound of any one of Exemplary Embodiment Nos. 39-65, wherein Z² is CH.
Exemplary Embodiment No. 68. The compound of any one of Exemplary Embodiment Nos. 39-64 and 66, wherein Z¹ and Z² are both N.
Exemplary Embodiment No. 69. The compound of any one of Exemplary Embodiment Nos. 39-68, wherein Z³ is C(O)NR²¹R²², SO₂NR²¹R²² or CO₂R²¹.
Exemplary Embodiment No. 70. The compound of any one of Exemplary Embodiment Nos. 39-69, wherein Z³ is C(O)NR²¹R²² or SO₂NR²¹R²².
Exemplary Embodiment No. 71. The compound of any one of Exemplary Embodiment Nos. 39-70, wherein Z³ is C(O)NR²¹R²².
Exemplary Embodiment No. 72. The compound of any one of Exemplary Embodiment Nos. 39-58 and 63-71, wherein Z⁴ is heterocyclyl.
Exemplary Embodiment No. 73. The compound of any one of Exemplary Embodiment Nos. 39-58 and 63-72, wherein Z⁴ is pyrrolidinyl, azetidinyl, morpholinyl or azaspiroheptyl.
Exemplary Embodiment No. 74. The compound of any one of Exemplary Embodiment Nos. 39-58 and 63-73, wherein Z⁴ is pyrrolidinyl, azetidinyl, morpholinyl or azaspiro[3.3.0]heptyl.
Exemplary Embodiment No. 75. The compound of any one of Exemplary Embodiment Nos. 39-58 and 63-74, wherein Z⁴ is pyrrolidinyl, azetidinyl, morpholinyl or azaspiro[3.3.0]heptyl, each optionally substituted with CN, OH, optionally substituted alkyl, aryl or heteroaryl.
Exemplary Embodiment No. 76. The compound of any one of Exemplary Embodiment Nos. 39-58 and 63-75, wherein Z⁴ is pyrrolidinyl, azetidinyl, morpholinyl or azaspiro[3.3.0]heptyl, each optionally substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl.
Exemplary Embodiment No. 77. The compound of any one of Exemplary Embodiment Nos. 39-58 and 63-76, wherein Z⁴ is azaspiro[3.3.0]heptyl substituted with phenyl.
Exemplary Embodiment No. 78. The compound of any one of Exemplary Embodiment Nos. 39-58 and 63-76, wherein Z⁴ is morpholinyl substituted with phenyl and methyl.
Exemplary Embodiment No. 79. The compound of any one of Exemplary Embodiment Nos. 39-58 and 63-76, wherein Z⁴ is azetidinyl substituted with (i) methyl or ethyl, and (ii) phenyl, 3-methylphenyl or 4-methylphenyl.
Exemplary Embodiment No. 80. The compound of any one of Exemplary Embodiment Nos. 39-58 and 63-76, wherein Z⁴ is pyrrolidinyl substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl.
Exemplary Embodiment No. 81. The compound of any one of Exemplary Embodiment Nos. 39-58, 63-76 and 80, wherein Z⁴ is

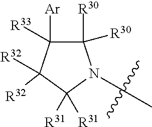

wherein each R³⁰ is H or the two R³⁰ form oxo;
   each R³¹ is H or the two R³¹ form oxo;
   each R³² is H or methyl;
   R³³ is optionally substituted alkyl, or together with R³¹ forms a bridged bicyclic pyrrolidinyl group; and
   Ar is an optionally substituted aryl or heteroaryl group, OH or CN.
Exemplary Embodiment No. 82. The compound of Exemplary Embodiment No. 81, wherein R³⁰, R³¹ and R³² is H.
Exemplary Embodiment No. 83. The compound of Exemplary Embodiment No. 81 or Exemplary Embodiment No. 82, wherein R³³ is optionally substituted alkyl.
Exemplary Embodiment No. 84. The compound of any one of Exemplary Embodiment Nos. 81-83, wherein R³³ is optionally substituted methyl.
Exemplary Embodiment No. 85. The compound of any one of Exemplary Embodiment Nos. 81-84, wherein R³³ is methyl.
Exemplary Embodiment No. 86. The compound of any one of Exemplary Embodiment Nos. 81-85, wherein Ar is aryl or heteroaryl optionally substituted with methyl, chloro, fluoro, methoxy or hydroxy.
Exemplary Embodiment No. 87. The compound of any one of Exemplary Embodiment Nos. 81-86, wherein Ar is phenyl, pyridyl, thiazolyl, oxazolyl, pyrazolyl, oxadiazolyl, tetrazolyl, thiadiazolyl or triazolyl, each optionally substituted with methyl, chloro, fluoro, methoxy or hydroxy.
Exemplary Embodiment No. 88. The compound of any one of Exemplary Embodiment Nos. 81-87, wherein Ar is phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl.
Exemplary Embodiment No. 89. The compound of any one of Exemplary Embodiment Nos. 81-88, wherein Ar is phenyl optionally substituted with methyl, chloro, fluoro, methoxy or hydroxy.
Exemplary Embodiment No. 90. The compound of any one of Exemplary Embodiment Nos. 81-89, wherein Ar is unsubstituted phenyl.
Exemplary Embodiment No. 91. The compound of any one of Exemplary Embodiment Nos. 81-90, wherein Z⁴ is

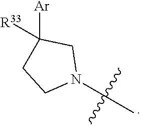

Exemplary Embodiment No. 92. The compound of any one of Exemplary Embodiment Nos. 81-91, wherein Z⁴ is

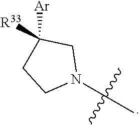

Exemplary Embodiment No. 93. The compound of any one of Exemplary Embodiment Nos. 81-92, wherein Z⁴ is

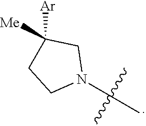

Exemplary Embodiment No. 94. The compound of any one of Exemplary Embodiment Nos. 81-93, wherein Z⁴ is

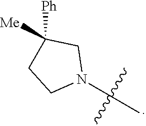 [SMILES: *N1CC[C@](C)(c2ccccc2)C1]

Exemplary Embodiment No. 95. The compound of any one of Exemplary Embodiment Nos. 39-94, wherein the compound has one of the following formulae:

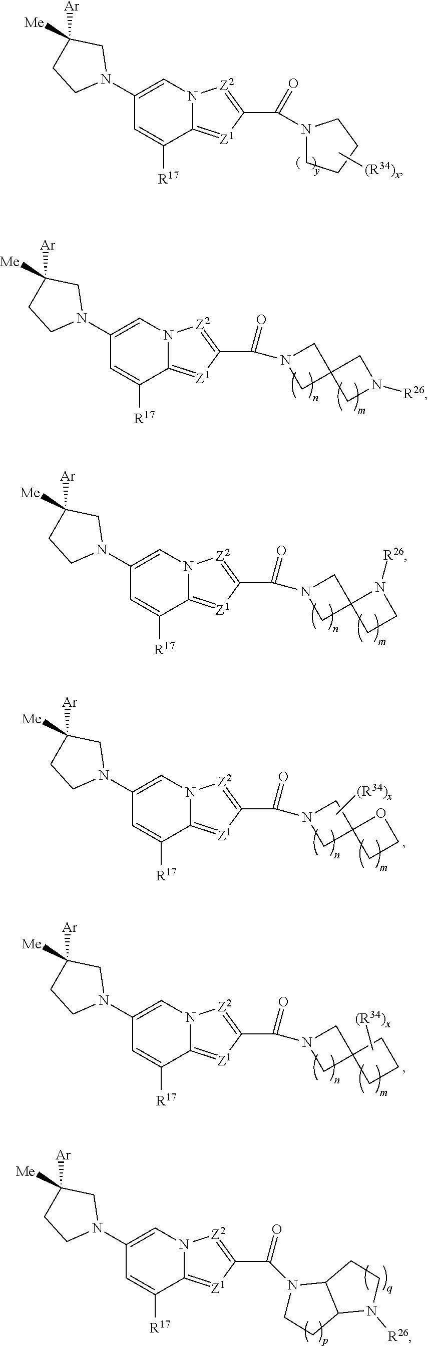

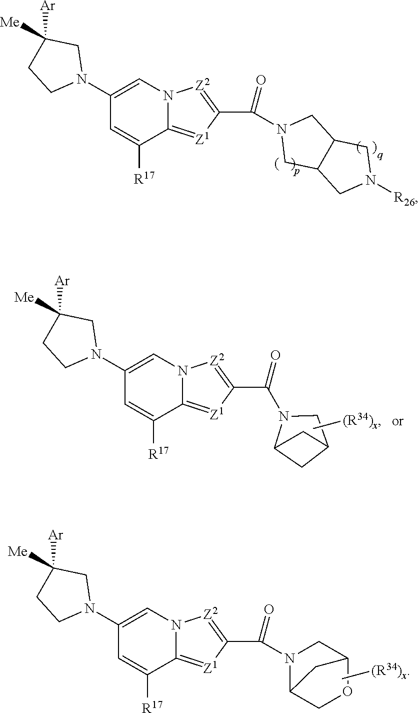

Exemplary Embodiment No. 96. A compound selected from the compounds provided in the Examples.
Exemplary Embodiment No. 97. A pharmaceutical composition, comprising the compound of any one of Exemplary Embodiment Nos. 1-96 and a pharmaceutically acceptable carrier.
Exemplary Embodiment No. 98. A method of treating light chain amyloidosis, comprising administering to a subject a compound of any one of Exemplary Embodiment Nos. 1-96 or a composition of Exemplary Embodiment No. 97.
Exemplary Embodiment No. 99. A method of stabilizing immunoglobulin light chains, comprising contacting the immunoglobulin light chains with a compound of any one of Exemplary Embodiment Nos. 1-96.
Exemplary Embodiment No. 100. The method of Exemplary Embodiment No. 99, wherein the immunoglobulin light chains are stabilized in a native conformation thereof.
Exemplary Embodiment No. 101. The method of Exemplary Embodiment No. 99 or Exemplary Embodiment No. 100, wherein the immunoglobulin light chains are dimers.
Exemplary Embodiment No. 102. A method of preventing or lessening immunoglobulin light chain misfolding and/or endoproteolysis, comprising contacting the immunoglobulin light chains with a compound of any one of Exemplary Embodiment Nos. 1-96.
Exemplary Embodiment No. 103. A method of maintenance therapy upon recurrence of light chain amyloidosis following primary treatment, comprising administering to a subject a compound of any one of Exemplary Embodiment Nos. 1-96 or a composition of Exemplary Embodiment No. 97.
Exemplary Embodiment No. 104. The method of Exemplary Embodiment No. 98 or Exemplary Embodiment No. 103, further comprising administering to the subject a second active agent selected from proteasome inhibitors (e.g., bortezomib, ixazomib, carfilzomib), alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129), an agent that promotes amyloid deposit clearance (e.g., CAEL-101, birtamimab), an anti-thymocyte antibody (e.g., Thymoglobulin®, Atgam®), atacicept and/or an anti-amyloid antibody.
Exemplary Embodiment No. 105. The method of any one of Exemplary Embodiment Nos. 98, 103 and 104, further comprising stem cell transplant therapy.
Exemplary Embodiment No. 106. The method of claim 104 or claim 105, wherein the second active agent is a plasma cell-directed therapy.

### EXAMPLES

The examples below are meant to illustrate some embodiments provided herein, and not to limit the scope of this disclosure.

It is understood that the values described in the examples are approximate and subject to experimental and instrumental variations.

### Example 1

### 2-methyl-8-(8-methyl-6-((R)-3-methyl-3-phenylpyrrolidin-1-yl)-[1,2,4]triazolo[1,5-a]pyridin-2-yl)-8,9-dihydro-5,8-methano[1,2,4]triazolo[1,5-d][1,4]oxazepin-5(6H)-amine (Compound 1B)

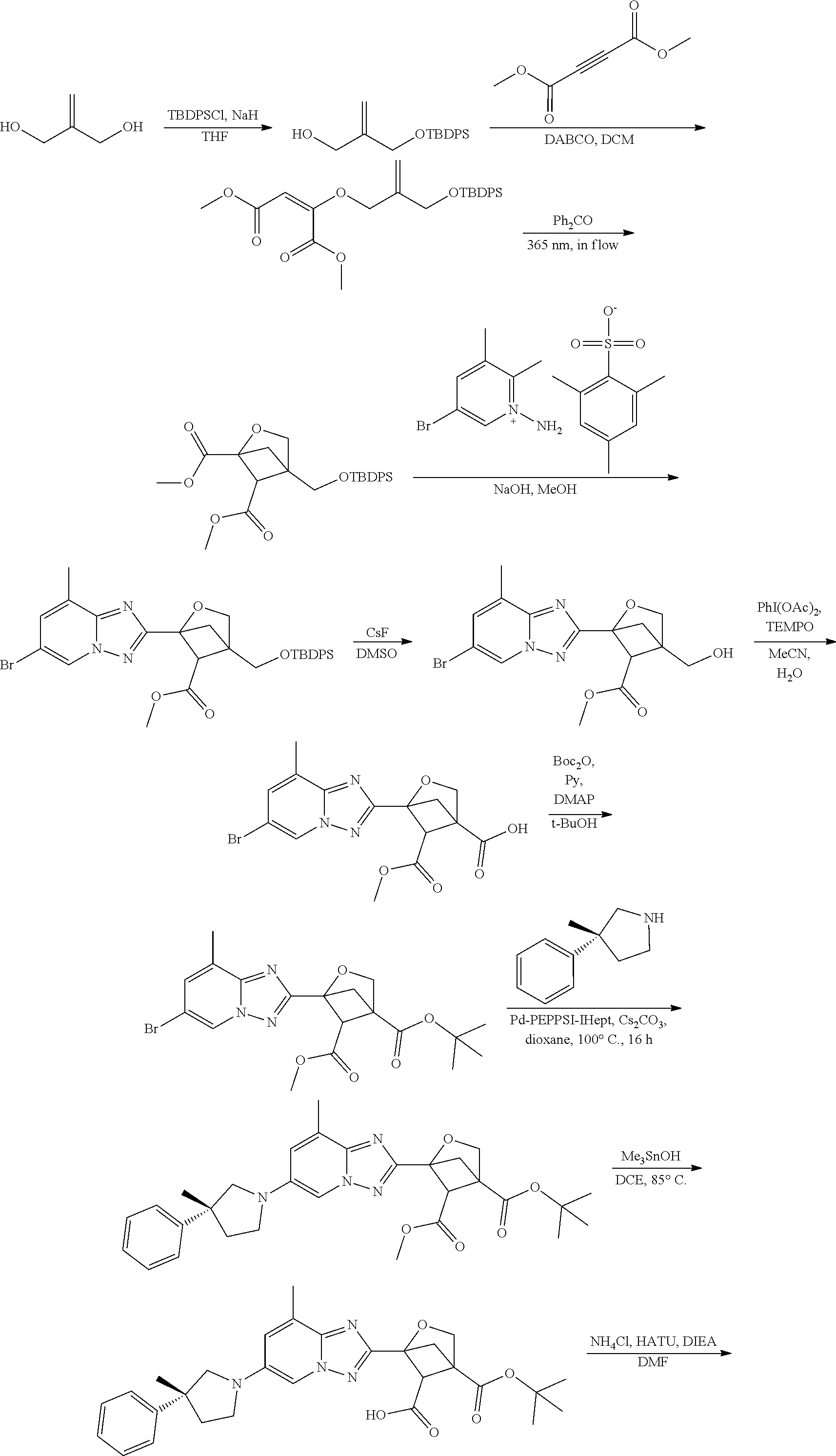

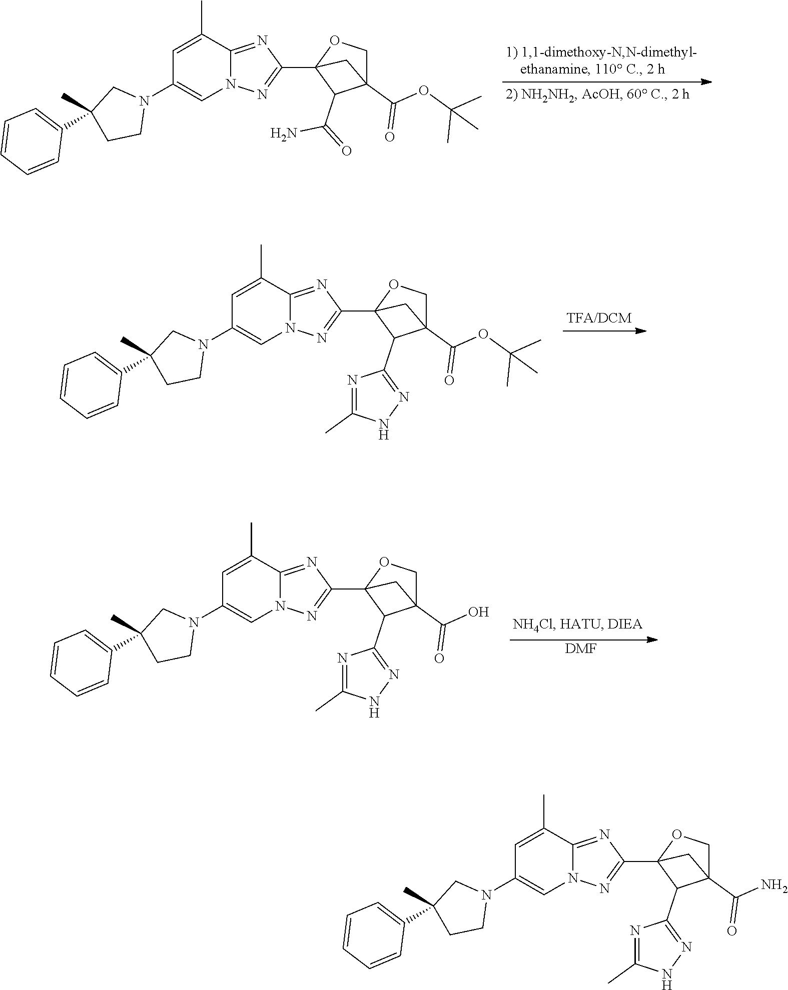

### Step 1: 2-(((tert-butyldiphenylsilyl)oxy)methyl)prop-2-en-1-ol

To a solution of 2-methylenepropane-1,3-diol (20 g, 227.00 mmol, 1 eq) in THF (200 mL) was added NaH (9.08 g, 227.00 mmol, 60% purity, 1 eq) at 0° C., the mixture was stirred at 0° C. for 1 h. Then TBDPSCl (68.63 g, 249.70 mmol, 63.90 mL, 1.1 eq) was added to the mixture at same temperature, the mixture was stirred at 25° C. for 4 h. TLC (SiO₂, PE:EtOAc=5:1, Rf=0.3) indicated reactant was consumed completely and one new spot was formed. The reaction mixture was quenched by H₂O (100 mL) at 0° C., then extracted with ethyl acetate (200 mL×3), the combined organic layers dried over anhydrous sodium sulfate, filtered and the filtrate was concentrated under reduced pressure to give a residue. The residue was purified by flash silica gel chromatography (ISCO®; 150 g SepaFlash® Silica Flash Column, Eluent of 0~15% Ethyl acetate/Petroleum ether gradient) to give compound 2-[[tert-butyl(diphenyl)silyl]oxymethyl]prop-2-en-1-ol (58 g, 78% yield) as yellow oil.

¹H NMR (400 MHz, CHLOROFORM-d) δ=7.69 (d, J=6.8 Hz, 4H), 7.49-7.34 (m, 6H), 5.14 (br d, J=15.0 Hz, 2H), 4.27 (s, 2H), 4.19 (s, 2H), 1.08 (s, 9H).

### Step 2: dimethyl 2-((2-(((tert-butyldiphenylsilyl)oxy)methyl)allyl)oxy)maleate

To a solution of dimethyl but-2-ynedioate (32.07 g, 225.68 mmol, 1 eq) and 2-[[tertbutyl(diphenyl)silyl]oxymethyl]prop-2-en-1-ol (70 g, 214.39 mmol, 0.95 eq) in DCM (700 mL) was added DABCO (5.06 g, 45.14 mmol, 0.2 eq). The mixture was stirred at 25° C. for 12 h. TLC (SiO2, PE:EtOAc=10:1, Rf=0.31) indicated reactant was consumed and one new spot formed. The reaction was filtered and the filtered concentrated under reduced pressure to give a residue. The residue was purified by flash silica gel chromatography (ISCO®; 220 g SepaFlash® Silica Flash Column, Eluent of 0-5% Ethyl acetate/Petroleum ether gradient to give compound dimethyl (E)-2-[2-[[tert-butyl(diphenyl)silyl]oxymethyl]allyloxy]but-2-enedioate (35 g, 33% yield) as a colorless oil. [2931]1H NMR (400 MHz, CHLOROFORM-d) δ=7.67 (br d, J=6.8 Hz, 4H), 7.47-7.36 (m, 6H), 6.16 (s, 1H), 5.38 (s, 1H), 5.27 (s, 1H), 4.70 (s, 2H), 4.30 (s, 2H), 3.77 (s, 3H), 3.64 (s, 3H), 1.06 (s, 9H).

### Step 3: dimethyl 4-(((tert-butyldiphenylsilyl)oxy)methyl)-2-oxabicyclo[2.1.1]hexane-1,5-dicarboxylate

To a solution of dimethyl (E)-2-[2-[[tert-butyl(diphenyl)silyl]oxymethyl]allyloxy]but-2-enedioate (45 g, 96.03 mmol, 1 eq) in ACN (800 mL) was added diphenylmethanone (1.75 g, 9.60 mmol, 0.1 eq). The mixture was stirred at 25° C. for 40 min under 365 nm. TLC (SiO₂, PE:EtOAc=3:1) indicated reactant was consumed completely and some new spots formed. The reaction was filtered and the filtered concentrated under reduced pressure to give a residue. The residue was purified by flash silica gel chromatography (ISCO®; 120 g SepaFlash® Silica Flash Column, Eluent of 0~18% Ethyl acetate/Petroleum ether gradient) to give compound dimethyl 4-[[tert-butyl(diphenyl)silyl]oxymethyl]-2-oxabicyclo[2.1.1]hexane-1,5-dicarboxylate (7 g, 15% yield) as a colorless oil.

¹H NMR (400 MHz, CHLOROFORM-d) δ=7.69-7.61 (m, 4H), 7.48-7.38 (m, 6H), 4.07 (d, J=11.2 Hz, 1H), 3.93 (d, J=11.2 Hz, 1H), 3.90 (br d, J=6.0 Hz, 1H), 3.85 (s, 3H), 3.78 (d, J=6.0 Hz, 1H), 3.67 (s, 3H), 3.14 (s, 1H), 2.14 (d, J=7.6 Hz, 1H), 1.81 (d, J=7.6 Hz, 1H), 1.08 (s, 9H).

### Step 4: methyl 1-(6-bromo-8-methyl-[1,2,4]triazolo[1,5-a]pyridin-2-yl)-4-(((tert-butyldiphenylsilyl)oxy)methyl)-2-oxabicyclo[2.1.1]hexane-5-carboxylate

To a solution of 5-bromo-3-methyl-pyridin-1-ium-1,2-diamine;2,4,6-trimethylbenzenesulfonate (6.70 g, 16.64 mmol, 1.3 eq) and dimethyl 4-[[tert-butyl(diphenyl)silyl]oxymethyl]-2-oxabicyclo[2.1.1]hexane-1,5-dicarboxylate (6 g, 12.80 mmol, 1 eq) in MeOH (300 mL) was added NaOH (768.17 mg, 19.21 mmol, 1.5 eq). The mixture was stirred at 25° C. for 12 h. LCMS showed starting material was consumed and the desired mass was detected. The reaction was concentrated under reduced pressure to give a residue. The residue was purified by flash silica gel chromatography (ISCO®; 40 g SepaFlash® Silica Flash Column, Eluent of 0~20% Ethyl acetate/Petroleum ether gradient) to give compound methyl 1-(6-bromo-8-methyl-[1,2,4]triazolo[1,5-a]pyridin-2-yl)-4-[[tert-butyl(diphenyl)silyl]oxymethyl]- 2-oxabicyclo[2.1.1]hexane-5-carboxylate (4.1 g, 52% yield) as a yellow gum.

LC-MS [ESI, M+1]: 620.3.

¹H NMR (400 MHz, CHLOROFORM-d) δ=8.58 (s, 1H), 7.71-7.64 (m, 4H), 7.48-7.36 (m, 7H), 4.19-4.12 (m, 1H), 4.10 (d, J=6.0 Hz, 1H), 4.07-4.02 (m, 1H), 3.97-3.92 (m, 1H), 3.59 (s, 3H), 3.34 (s, 1H), 2.67 (s, 3H), 2.33 (d, J=7.6 Hz, 1H), 2.02 (d, J=7.6 Hz, 1H), 1.09 (s, 9H).

### Step 5: methyl 1-(6-bromo-8-methyl-[1,2,4]triazolo[1,5-a]pyridin-2-yl)-4-(hydroxymethyl)-2-oxabicyclo[2.1.1]hexane-5-carboxylate

To a solution of methyl 1-(6-bromo-8-methyl-[1,2,4]triazolo[1,5-a]pyridin-2-yl)-4-[[tertbutyl(diphenyl)silyl]oxymethyl]-2-oxabicyclo[2.1.1]hexane-5-carboxylate (4.7 g, 7.57 mmol, 1 eq) in DMSO (20 mL) and THF (10 mL) was added CsF (1.73 g, 11.36 mmol, 1.5 eq). The mixture was stirred at 25° C. for 12 h. LCMS showed starting material was consumed and one main peak with desired mass was detected. The mixture was quenched with H₂O (100 mL) and extracted with EtOAc (50 mL×3), the organic phase was dried, filtered, and concentrated under pressure to give the product. The residue was purified by flash silica gel chromatography (ISCO®; 40 g SepaFlash® Silica Flash Column, Eluent of 0~5% Methanol/Dichloromethane gradient @100 mL/min). The crude product was further triturated with ACN at 25° C. for 15 min and the solid was filtered to give compound methyl 1-(6-bromo-8-methyl-[1,2,4]triazolo[1,5-a]pyridin-2-yl)-4-(hydroxymethyl)-2-oxabicyclo[2.1.1]hexane-5-carboxylate (2 g, 69% yield) as a white solid.

LC-MS [ESI, M+1]: 381.8.

¹H NMR (400 MHz, CHLOROFORM-d) δ=8.58 (s, 1H), 7.43 (s, 1H), 4.23 (d, J=6.4 Hz, 1H), 4.19-4.06 (m, 2H), 3.99 (d, J=6.4 Hz, 1H), 3.65 (s, 3H), 3.36 (s, 1H), 2.68 (s, 3H), 2.27 (d, J=7.6 Hz, 1H), 2.04 (d, J=6.8 Hz, 1H).

### Step 6: 1-(6-bromo-8-methyl-[1,2,4]triazolo[1,5-a]pyridin-2-yl)-5-(methoxycarbonyl)-2-oxabicyclo[2.1.1]hexane-4-carboxylic acid

To a solution of methyl 1-(6-bromo-8-methyl-[1,2,4]triazolo[1,5-a]pyridin-2-yl)-4-(hydroxymethyl)-2-oxabicyclo[2.1.1]hexane-5-carboxylate (3 g, 7.85 mmol, 1 eq) in ACN (10 mL), H2O (10 mL) and acetone (10 mL) was added PIDA (6.57 g, 20.41 mmol, 2.6 eq) and TEMPO (493.72 mg, 3.14 mmol, 0.4 eq). The mixture was stirred at 50° C. for 3 h. LCMS showed starting material was consumed and one main peak with desired mass was detected. The mixture was quenched with H₂O (10 mL) and extracted with EtOAc (20 mL×3), the organic phase was dried, filtered, and concentrated under pressure to give the product. The crude product was further triturated with MTBE at 0° C. for 15 min and the solid was filtered to give compound 1-(6-bromo-8-methyl-[1,2,4]triazolo[1,5-a]pyridin-2-yl)-5-methoxycarbonyl-2-oxabicyclo[2.1.1]hexane4-carboxylic acid (2.6 g, 84% yield) as a white solid.

LC-MS [ESI, M+1]: 398.0.

¹H NMR (400 MHz, CHLOROFORM-d) δ=8.60 (s, 1H), 7.45 (s, 1H), 4.51-4.35 (m, 1H), 4.25 (d, J=6.4 Hz, 1H), 3.78-3.73 (m, 1H), 3.71-3.60 (m, 3H), 2.73-2.62 (m, 4H), 2.40-2.30 (m, 1H).

### Step 7: 4-(tert-butyl) 5-methyl 1-(6-bromo-8-methyl-[1,2,4]triazolo[1,5-a]pyridin-2-yl)-2-oxabicyclo[2.1.1]hexane-4,5-dicarboxylate

To a solution of 1-(6-bromo-8-methyl-[1,2,4]triazolo[1,5-a]pyridin-2-yl)-5-methoxycarbonyl-2-oxabicyclo[2.1.1]hexane-4-carboxylic acid (3.5 g, 8.83 mmol, 1 eq) in t-BuOH (30 mL) was added Boc₂O (38.56 g, 176.68 mmol, 40.59 mL, 20 eq), DMAP (1.08 g, 8.83 mmol, 1 eq) and pyridine (13.98 g, 176.68 mmol, 20 eq). The mixture was stirred at 25° C. for 6 h. LCMS showed starting material was consumed and one main peak with desired mass was detected. The mixture was quenched with H₂O (15 mL) and extracted with EtOAc (20 mL×3), the organic phase was washed with saturated NaCl (20 mL×3), dried, filtered, and concentrated under pressure to give the product. The residue was purified by flash silica gel chromatography (ISCO®; 80 g SepaFlash® Silica Flash Column, Eluent of 0~45% Ethyl acetate/Petroleum ether gradient) to give compound 04-tert-butyl 05-methyl 1-(6-bromo-8-methyl-[1,2,4]triazolo[1,5-a]pyridin-2-yl)-2-oxabicyclo[2.1.1]hexane-4,5-dicarboxylate (2.3 g, 57% yield) as a brown gum.

LC-MS [ESI, M+1]: 455.2.

¹H NMR (400 MHz, CHLOROFORM-d) δ=8.49 (d, J=1.0 Hz, 1H), 7.33 (d, J=1.2 Hz, 1H), 4.27 (d, J=6.4 Hz, 1H), 4.08 (d, J=6.4 Hz, 1H), 3.56 (s, 3H), 3.53 (s, 1H), 2.58 (s, 3H), 2.49 (d, J=7.2 Hz, 1H), 2.18 (dd, J=1.0, 7.2 Hz, 1H), 1.45 (s, 9H).

### Step 8: 4-(tert-butyl) 5-methyl 1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4,5-dicarboxylate

A mixture of (3R)-3-methyl-3-phenyl-pyrrolidine (901.94 mg, 5.59 mmol, 1.1 eq),04-tert-butyl 05-methyl 1-(6-bromo-8-methyl-[1,2,4]triazolo[1,5-a]pyridin-2-yl)-2-oxabicyclo[2.1.1]hexane-4,5-dicarboxylate (2.3 g, 5.09 mmol, 1 eq), Pd-PEPPSI-IHeptCl (445.20 mg, 457.66 mol, 0.09 eq) and Cs₂CO₃ (3.31 g, 10.17 mmol, 2 eq) in dioxane (30 mL) was degassed and purged with N₂ for 3 times, and then the mixture was stirred at 100° C. for 10 h under N₂ atmosphere. LCMS showed starting material was consumed and one main peak with desired mass was detected. The mixture was filtered, and concentrated under pressure to give the product. The residue was purified by flash silica gel chromatography (ISCO®; 40 g SepaFlash® Silica Flash Column, Eluent of 0~38% Ethylacetate/Petroleum ether gradient) to give compound 04-tert-butyl 05-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (2.2 g, 81% yield) as a yellow solid.

LC-MS [ESI, M+1]: 533.2. [2954]¹H NMR (400 MHz, CHLOROFORM-d) δ=7.68 (d, J=1.8 Hz, 1H), 7.41-7.32 (m, 4H), 7.28 (br s, 1H), 6.96 (s, 1H), 4.37 (d, J=6.4 Hz, 1H), 4.16 (d, J=6.4 Hz, 1H), 3.65 (s, 3H), 3.63-3.58 (m, 2H), 3.56-3.45 (m, 2H), 3.44-3.37 (m, 1H), 2.66 (s, 3H), 2.56 (d, J=7.2 Hz, 1H), 2.39 (td, J=8.0, 12.0 Hz, 1H), 2.31-2.23 (m, 2H), 1.54 (s, 9H), 1.48 (s, 3H).

### Step 9: 4-(tert-butoxycarbonyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic acid

To a solution of 04-tert-butyl 05-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (600 mg, 1.13 mmol, 1 eq) in DCE (10 mL) was added hydroxy(trimethyl)stannane (1.02 g, 5.63 mmol, 5 eq). The mixture was stirred at 80° C. for 12 h. LCMS showed starting material was consumed and one main peak with desired mass was detected. The mixture was concentrated in vaco. The residue was purified by flash silica gel chromatography (ISCO®; 12 g SepaFlash® Silica Flash Column, Eluent of 0~60% Ethyl acetate/Petroleum ether gradient) to give compound 4-tert-butoxycarbonyl-1-[8-methyl-6-[(3R)-3-methyl-3-phenyl-pyrrolidin-1-yl]-[1,2,4]triazolo[1,5-a]pyridin2-yl]-2-oxabicyclo[2.1.1]hexane-5-carboxylic acid (500 mg, 85% yield) as yellow solid.

LC-MS [ESI, M+1]: 519.3.

### Step 10: tert-butyl 5-carbamoyl-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylate

To a solution of 4-tert-butoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.42 g, 2.74 mmol, 1 eq) and NH₄C₁ (732.33 mg, 13.69 mmol, 5 eq) in DMF (10 mL) was added HATU (1.56 g, 4.11 mmol, 1.5 eq) and DIEA (1.06 g, 8.21 mmol, 1.43 mL, 3 eq). The mixture was stirred at 25° C. for 1 h. LCMS showed starting material was consumed and one main peak with desired mass was detected. The mixture was filtered, and concentrated under pressure to give the product. The residue was purified by prep-HPLC purification (mobile phase: [water(FA)-ACN]; B %: 60%-70%,6 min), the mixture was concentrated and lyophilized to give compound tert-butyl 5-carbamoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (1.02 g, 72% yield) as a green solid.

LC-MS [ESI, M+1]: 518.2.

¹H NMR (400 MHz, CHLOROFORM-d) δ=7.64 (d, J=1.6 Hz, 1H), 7.43-7.30 (m, 4H), 7.28 (s, 1H), 7.00 (s, 1H), 5.51 (br s, 1H), 4.28 (d, J=6.4 Hz, 1H), 4.12 (d, J=6.4 Hz, 1H), 3.61 (d, J=8.8 Hz, 1H), 3.50 (br t, J=9.2 Hz, 2H), 3.41 (br d, J=3.8 Hz, 1H), 3.38 (s, 1H), 2.68-2.56 (m, 3H), 2.46-2.34 (m, 3H), 2.33-2.22 (m, 1H), 1.53 (s, 8H), 1.48 (s, 3H).

### Step 11: tert-butyl 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylate

To a solution of tert-butyl 5-carbamoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (0.8 g, 1.55 mmol, 1 eq) in 1,1-dimethoxy-N,N-dimethylethanamine (24.14 g, 26.50 mL), the mixture was stirred at 110° C. for 2 h. Then the mixture was concentrated under reduced pressure to give a residue. To a solution of this residue in EtOH (10 mL) was added acetic acid (1.86 g, 30.91 mmol, 1.77 mL, 20 eq) and hydrazine;hydrate (910.24 mg, 15.46 mmol, 882.01 L, 85% purity, 10 eq) at −10° C. And then the mixture was stirred at 25° C. for 1 h. LCMS showed starting material was consumed and one main peak with desired mass was detected. The mixture was quenched with water (15 mL) and extracted with ethyl acetate (10 mL×3), the combined organic phase was dried over anhydrous sodium sulfate, filtered and concentrated to give a residue. The residue was purified by flash silica gel chromatography (ISCO®; 40 g SepaFlash® Silica Flash Column, Eluent of 0~8% Methanol/Dichloromethane gradient) to give compound tert-butyl 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate (805 mg, 93% yield) as a brown solid.

LC-MS [ESI, M+1]: 556.3.

### Step 12: 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylic acid (Compound 1A)

A solution of tert-butyl 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate (45.00 mg, 80.98 mol, 1 eq) in TFA (0.1 mL), DCM (0.5 mL) was stirred at 25° C. for 1 h. LCMS showed starting material was consumed and one main peak with desired mass was detected. The mixture was concentrated in vaco. The residue was purified by prep-HPLC(column: Phenomenex luna C18 150*25 mm*10 um; mobile phase: [water(FA)-ACN]; gradient:28%-58% B over 10 min) to give 1-[8-methyl-6-[(3R)-3-methyl-3-phenyl-pyrrolidin-1-yl]-[1,2,4]triazolo[1,5-a]pyridin-2-yl]-5-(5-methyl-1H1,2,4-triazol-3-yl)-2-oxabicyclo[2.1.1]hexane-4-carboxylic acid (36 mg, 90% yield) as white solid.

LC-MS [ESI, M+1]: 500.2.

¹H NMR (CHLOROFORM-d, 400 MHz) δ 7.67 (s, 1H), 7.4-7.4 (m, 2H), 7.3-7.4 (m, 3H), 7.04 (s, 1H), 4.17 (br d, 1H, J=6.4 Hz), 3.92 (br d, 1H, J=6.0 Hz), 3.82 (br s, 1H), 3.63 (br d, 1H, J=8.8 Hz), 3.5-3.6 (m, 2H), 3.4-3.5 (m, 1H), 2.77 (br d, 1H, J=6.0 Hz), 2.67 (s, 3H), 2.49 (s, 3H), 2.4-2.4 (m, 1H), 2.2-2.3 (m, 2H), 1.49 (s, 3H).

### Step 13: 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxamide

To a solution of 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylic acid (25 mg, 50.04 mol, 1 eq) in DMF (1 mL) was added NH₄C₁ (26.77 mg, 500.44 mol, 10 eq), HATU (28.54 mg, 75.07 mol, 1.5 eq), DIEA (32.34 mg, 250.22 mol, 5 eq). The mixture was stirred at 25° C. for 1 h. LCMS showed starting material was consumed and one main peak with desired mass was detected. The mixture was filtered. The residue was purified by prep-HPLC (column: Phenomenex luna C18 150*25 mm*10 um; mobile phase: [water(FA)-ACN]; gradient:26%-56% B over 10 min) to give compound 1-[8-methyl-6-[(3R)-3-methyl-3-phenyl-pyrrolidin-1-yl]-[1,2,4]triazolo[1,5-a]pyridin-2-yl]-5-(5-methyl-1H1,2,4-triazol-3-yl)-2-oxabicyclo[2.1.1]hexane-4-carboxamide (7.98 mg, 32% yield) as a white solid.

LC-MS [ESI, M+1]: 499.2.

¹H NMR (400 MHz, CHLOROFORM-d) δ=9.17 (br s, 1H), 7.66 (s, 1H), 7.41-7.34 (m, 2H), 7.34-7.27 (m, 3H), 7.03 (s, 1H), 5.87-5.54 (m, 1H), 4.12 (d, J=6.0 Hz, 1H), 3.86 (d, J=6.0 Hz, 1H), 3.65-3.58 (m, 2H), 3.57-3.46 (m, 2H), 3.45-3.37 (m, 1H), 2.71-2.59 (m, 5H), 2.45 (s, 3H), 2.42-2.34 (m, 1H), 2.32-2.22 (m, 1H), 1.47 (s, 3H)

Following compounds were synthesized according to this example.

| Cmpd. No. | Synthesis | LC/MS | ¹H NMR |
|---|---|---|---|
| 41 | Similar to | m/z 513.4 | ¹H NMR (400 MHz, CHLOROFORM-d) δ = 8.91 (br |
| | Example 1 | [M + H]⁺ | d, J = 2.9 Hz, 1H), 7.66 (s, 1H), 7.41-7.34 (m, 2H), |
| | | | 7.34-7.27 (m, 3H), 7.04 (s, 1H), 4.10 (d, J = 6.0 Hz, |
| | | | 1H), 3.78 (d, J = 6.0 Hz, 1H), 3.61 (d, J = 8.8 Hz, 1H), |
| | | | 3.58-3.45 (m, 3H), 3.45-3.37 (m, 1H), 2.95 (d, J = |
| | | | 4.6 Hz, 3H), 2.71-2.64 (m, 4H), 2.63 (s, 1H), 2.48 (s, |
| | | | 3H), 2.42-2.35 (m, 1H), 2.31-2.25 (m, 1H), 1.47 (s, 3H) |
| 42 | Similar to | m/z 448.1 | ¹H NMR (400 MHz, CHLOROFORM-d) δ ppm 1.47 |
| | Example 1 | [M + H]⁺ | (s, 3 H) 2.04 (d, J = 7.6 Hz, 1 H) 2.21 (d, J = 7.6 Hz, 1 H) |
| | | | 2.27 (ddd, J = 12.0, 7.16, 4.00 Hz, 1 H) 2.39 (dt, J = 12.0, |
| | | | 8.35 Hz, 1 H) 2.60 (s, 3 H) 3.02 (s, 1 H) 3.36-3.44 (m, |
| | | | 1 H) 3.46-3.56 (m, 2 H) 3.60 (br d, J = 8.8 Hz, 1 H) |
| | | | 3.93 (d, J = 6.8 Hz, 1 H) 4.00 (d, J = 12.0 Hz, 1 H) 4.16 |
| | | | (d, J = 11.76 Hz, 1 H) 4.26 (d, J = 6.8 Hz, 1 H) 5.81 (br s, |
| | | | 1 H) 7.00 (s, 1 H) 7.25 (br s, 1 H) 7.27-7.34 (m, 3 H) |
| | | | 7.34-7.40 (m, 2 H) 7.63 (s, 1 H) 9.19 (br s, 1 H) |
| 43 | Similar to | m/z 486.2 | ¹H NMR (400 MHz, CHLOROFORM-d) δ ppm 1.47 |
| | Example 1 | [M + H]⁺ | (s, 3 H) 2.25-2.31 (m, 1 H) 2.31-2.41 (m, 3 H) 2.43 |
| | | | (s, 3 H) 2.65 (s, 3 H) 3.33-3.46 (m, 2 H) 3.48-3.57 |
| | | | (m, 2 H) 3.61 (d, J = 8.8 Hz, 1 H) 3.88 (s, 2 H) 4.09 (d, |
| | | | J = 12.0 Hz, 1 H) 4.27 (d, J = 12.0 Hz, 1 H) 7.02 (s, 1 H) |
| | | | 7.29 (br s, 1 H) 7.30-7.34 (m, 2 H) 7.35-7.40 (m, 2 |
| | | | H) 7.65 (d, J = 1.6 Hz, 1 H) |
| 44 | Similar to | m/z 447.2 | ¹H NMR (400 MHz, CHLOROFORM-d) δ ppm 1.46 |
| | Example 1 | [M + H]⁺ | (s, 3 H) 2.07-2.14 (m, 2 H) 2.22-2.29 (m, 1 H) 2.38 |
| | | | (dt, J = 12.0, 8.27 Hz, 1 H) 2.60 (s, 3 H) 2.95 (s, 1 H) |
| | | | 3.21-3.32 (m, 2 H) 3.39 (td, J = 8.63, 3.63 Hz, 1 H) |
| | | | 3.45-3.55 (m, 2 H) 3.60 (d, J = 8.88 Hz, 1 H) 3.90 (d, |
| | | | J = 6.25 Hz, 1 H) 4.10 (d, J = 6.13 Hz, 1 H) 5.60 (br s, 1 |
| | | | H) 6.98 (s, 1 H) 7.23-7.27 (m, 1 H) 7.29-7.33 (m, 2 |
| | | | H) 7.33-7.41 (m, 2 H) 7.63 (s, 1 H) 8.57 (br s, 1 H) |
| 45 | Similar to | m/z 461.2 | ¹H NMR (400 MHz, DMSO-d₆) δ ppm 1.35 (s, 3 H) |
| | Example 1 | [M + H]⁺ | 2.11 (d, J = 7.6 Hz, 1 H) 2.19 (d, J = 7.6 Hz, 1 H) 2.25 (br |
| | | | t, J = 6.94 Hz, 2 H) 2.51 (br s, 3 H) 3.41-3.43 (m, 1 H) |
| | | | 3.44-3.54 (m, 3 H) 3.56-3.61 (m, 1 H) 3.85-3.95 |
| | | | (m, 2 H) 7.20-7.26 (m, 1 H) 7.27-7.42 (m, 6 H) 7.63 |
| | | | (br s, 1 H) 7.96 (s, 1 H) 8.11 (br s, 1 H) 8.41 (br s, 1 H) |
| 46 | Similar to | m/z 485.2 | ¹H NMR (400 MHz, CHLOROFORM-d) δ ppm 1.47 |
| | Example 1 | [M + H]⁺ | (s, 3 H) 2.23-2.30 (m, 2 H) 2.33-2.40 (m, 2 H) 2.41 |
| | | | (s, 3 H) 2.65 (s, 3 H) 3.31-3.38 (m, 3 H) 3.39 (br s, 1 |
| | | | H) 3.45-3.56 (m, 2 H) 3.60 (d, J = 8.8 Hz, 1 H) 3.73 (d, |
| | | | J = 6.4 Hz, 1 H) 3.87 (d, J = 6.4 Hz, 1 H) 7.00 (s, 1 H) |
| | | | 7.25 (br s, 1 H) 7.29-7.33 (m, 2 H) 7.34-7.41 (m, 2 |
| | | | H) 7.64 (s, 1 H) |

### Example 2

### N-(2-methyl-8-(8-methyl-6-((R)-3-methyl-3-phenylpyrrolidin-1-yl)-[1,2,4]triazolo[1,5-a]pyridin-2-yl)-8,9-dihydro-5,8-methano[1,2,4]triazolo[1,5-d][1,4]oxazepin-5(6H)-yl)acetamide (Compound 2)

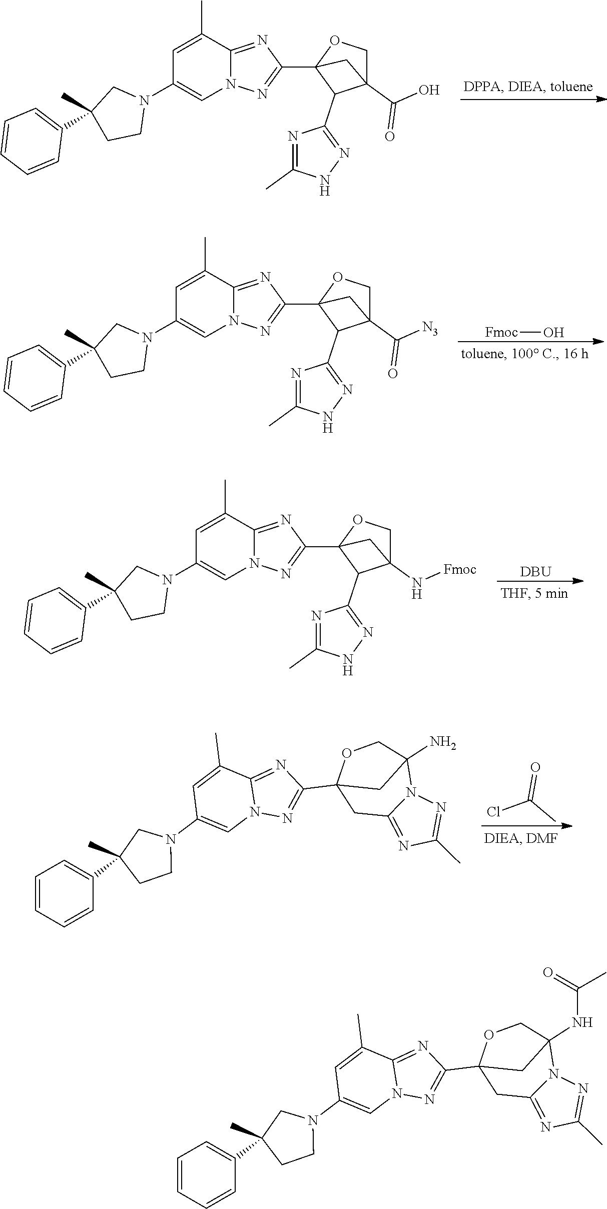

### Step 1: 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carbonyl azide

To a solution of 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylic acid (240 mg, 480.42 mol, 1 eq) in toluene (4 mL) was added DIEA (186.27 mg, 1.44 mmol, 251.04 μL, 3 eq), DPPA (198.32 mg, 720.63 mol, 1.5 eq). The mixture was stirred at 25° C. for 1 h. LCMS showed starting material was consumed and one main peak with desired mass was detected. The mixture was quenched with water (2 mL) and extracted with EtOAc (2 mL×3), the combined organic phase was dried over anhydrous sodium sulfate, filtered and concentrated to give a residue.

LC-MS [ESI, M+1]: 525.1.

### Step 2: (9H-fluoren-9-yl)methyl ((1S,4S,5R)-5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)carbamate

To a solution of 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carbonyl azide (200 mg, 381.26 mol, 1 eq) in toluene (3 mL) was added 9H-fluoren-9-ylmethanol (374.10 mg, 1.91 mmol, 5 eq). The mixture was stirred at 120° C. for 5 min. LCMS showed starting material was consumed and one main peak with desired mass was detected. The mixture was concentrated in vacuo. The residue was purified by prep-HPLC(column: CD04-Welch Ultimate C18 150*25*7 um; mobile phase: [water(FA)-ACN]; gradient:50%-80% B over 8 min) to give 9H-fluoren-9-ylmethyl N-[1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]carbamate (105 mg, 40% yield) as white solid.

LC-MS [ESI, M+1]: 693.2. [2981]¹H NMR (400 MHz, CHLOROFORM-d) δ=7.74-7.47 (m, 4H), 7.39-7.14 (m, 10H), 6.93 (br s, 1H), 4.35 (br s, 2H), 4.16 (br t, J=6.4 Hz, 1H), 4.08-3.94 (m, 1H), 3.80-3.64 (m, 1H), 3.62-3.49 (m, 2H), 3.48-3.38 (m, 2H), 3.33 (br dd, J=5.2, 8.8 Hz, 1H), 2.96-2.78 (m, 1H), 2.69-2.47 (m, 4H), 2.42-2.25 (m, 4H), 2.23-2.13 (m, 1H), 1.39 (s, 3H).

### Step 3: 2-methyl-8-(8-methyl-6-((R)-3-methyl-3-phenylpyrrolidin-1-yl)-[1,2,4]triazolo[1,5-a]pyridin-2-yl)-8,9-dihydro-5,8-methano[1,2,4]triazolo[1,5-d][1,4]oxazepin-5(6H)-amine

To a solution of 9H-fluoren-9-ylmethyl N-[1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]carbamate (50 mg, 72.17 mol, 1 eq) in THF (1 mL) was added DBU (0.1 M, 0.5 eq). The mixture was stirred at 25° C. for 5 min. LCMS showed starting material was consumed and one main peak with desired mass was detected. The mixture was filtered. The residue was purified by prep-HPLC (column: CD02-Waters Xbidge BEH C18 150*25*10 um; mobile phase: [water(NH₃H₂O)-ACN]; gradient:30%-60% B over 10 min) to give 4-methyl-8-[8-methyl-6-[(3R)-3-methyl-3-phenyl-pyrrolidin-1-yl]-[1,2,4]triazolo[1,5-a]pyridin-2-yl]-9-oxa2,3,5-triazatricyclo[6.2.1.02,6]undeca-3,5-dien-1-amine (25 mg, 36% yield) as white solid.

LC-MS [ESI, M+1]: 471.2.

¹H NMR (400 MHz, METHANOL-d4) δ=7.79 (d, J=1.6 Hz, 1H), 7.43-7.30 (m, 4H), 7.27-7.17 (m, 2H), 4.17 (d, J=8.0 Hz, 1H), 4.00-3.91 (m, 1H), 3.62-3.51 (m, 5H), 3.42 (br d, J=4.0 Hz, 1H), 3.12 (d, J=13.2 Hz, 1H), 2.70 (d, J=12.0 Hz, 1H), 2.61 (s, 3H), 2.45-2.23 (m, 5H), 1.45 (s, 3H).

### Step 4: N-(2-methyl-8-(8-methyl-6-((R)-3-methyl-3-phenylpyrrolidin-1-yl)-[1,2,4]triazolo[1,5-a]pyridin-2-yl)-8,9-dihydro-5,8-methano[1,2,4]triazolo[1,5-d][1,4]oxazepin-5(6H)-yl)acetamide

To a solution of 4-methyl-8-[8-methyl-6-[(3R)-3-methyl-3-phenyl-pyrrolidin-1-yl]-[1,2,4]triazolo[1,5-a]pyridin-2-yl]-9-oxa-2,3,5-triazatricyclo[6.2.1.02,6]undeca-3,5-dien-1-amine (15 mg, 31.88 mol, 1 eq), Et3N (9.68 mg, 95.63 mol, 3 eq) in DMF (0.5 mL) was added acetyl chloride (5.00 mg, 63.75 mol, 2 eq) at 0° C. The mixture was stirred at 25° C. for 0.5 h. LCMS showed starting material was consumed and one main peak with desired mass was detected. The mixture was quenched with water (0.1 mL) and then filtered. The residue was purified by prep-HPLC(column: Waters xbridge 150*25 mm 10 um; mobile phase: [water(NH4HCO3)-ACN]; gradient:28%-58% B over 10 min) to give N-[4-methyl-8-[8-methyl-6-[(3R)-3-methyl-3-phenyl-pyrrolidin-1-yl]-[1,2,4]triazolo[1,5-a]pyridin-2-yl]-9-oxa-2,3,5-triazatricyclo[6.2.1.02,6]undeca-3,5-dien-1-yl]acetamide (3.19 mg, 19% yield) as white solid.

LC-MS [ESI, M+1]: 513.2.

¹H NMR (400 MHz, CHLOROFORM-d) δ=7.66 (d, J=1.6 Hz, 1H), 7.40-7.27 (m, 5H), 6.98 (br s, 2H), 4.55 (d, J=8.0 Hz, 1H), 4.24 (d, J=8.0 Hz, 1H), 3.74-3.66 (m, 1H), 3.64-3.51 (m, 4H), 3.50-3.46 (m, 1H), 3.40 (dt, J=3.6, 8.8 Hz, 1H), 3.16 (br d, J=12.0 Hz, 1H), 2.65 (s, 3H), 2.42 (s, 3H), 2.37 (s, 1H), 2.30-2.23 (m, 1H), 2.19 (s, 3H), 1.47 (s, 3H).

| Cmpd. | | | |
|---|---|---|---|
| No. | Synthesis | LC/MS | ¹HNMR |
| 47 | Similar to | m/z 433.1 | ¹H NMR (400 MHz, METHANOL-d₄) |
| | Example 2 | [M + H]⁺ | δ = 7.74 (d, J = 1.8 Hz, 1H), 7.41-7.29 |
| | | | (m, 4H), 7.25-7.17 (m, 2H), 4.07- |
| | | | 4.03 (m, 1H), 4.02-3.97 (m, 1H), 3.61- |
| | | | 3.50 (m, 3H), 3.40 (dt, J = 3.6, 8.6 Hz, |
| | | | 1H), 3.09-2.93 (m, 2H), 2.80 (dd, J = |
| | | | 1.6, 11.6 Hz, 1H), 2.59 (s, 3H), 2.42- |
| | | | 2.22 (m, 3H), 1.44 (s, 3H) |
| 48 | Similar to | m/z 71.2 | ¹H NMR (400 MHz, METHANOL-d₄) |
| | Example 2 | [M + H]⁺ | δ = 7.79 (s, 1H), 7.42-7.31 (m, 4H), |
| | | | 7.27-7.17 (m, 2H), 4.17 (d, J = 7.8 Hz, |
| | | | 1H), 4.01-3.93 (m, 1H), 3.61-3.53 |
| | | | (m, 4H), 3.42 (br dd, J = 5.6, 8.6 Hz, |
| | | | 1H), 3.11 (d, J = 12.0 Hz, 1H), 2.71 (br |
| | | | d, J = 10.6 Hz, 1H), 2.61 (s, 3H), 2.44- |
| | | | 2.33 (m, 4H), 2.33-2.27 (m, 1H), 1.45 |
| | | | (s, 3H) |

### Example 3

### 2-[1-[8-methyl-6-[rac-(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]propan-2-ol (Compound 3)

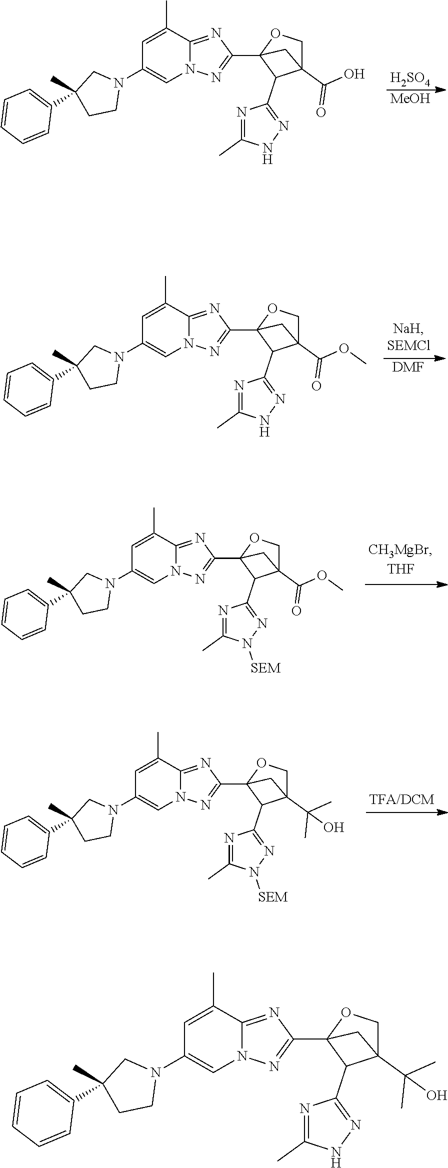

### Step 1: methyl 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate

To a solution of 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylic acid (200 mg, 400.35 mol, 1 eq) in MeOH (5 mL) was added H₂SO₄ (392.66 mg, 4.00 mmol, 213.40 μL, 10 eq). The mixture was stirred at 60° C. for 1 h. LCMS showed starting material was consumed and one main peak with desired mass was detected. The mixture was concentrated under pressure to give compound methyl 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate (180 mg, 88% yield) as yellow solid.

LC-MS [ESI, M+1]: 514.2.

¹H NMR (400 MHz, CHLOROFORM-d) δ=7.65 (d, J=1.6 Hz, 1H), 7.42-7.35 (m, 2H), 7.34-7.29 (m, 2H), 7.28-7.25 (m, 1H), 7.02 (s, 1H), 4.17 (d, J=6.8 Hz, 1H), 3.99 (d, J=6.4 Hz, 1H), 3.93-3.83 (m, 4H), 3.62 (d, J=8.8 Hz, 1H), 3.56-3.48 (m, 2H), 3.42 (td, J=4.4, 8.8 Hz, 1H), 2.76 (d, J=7.6 Hz, 1H), 2.70-2.61 (m, 3H), 2.56 (d, J=7.6 Hz, 1H), 2.44 (s, 3H), 2.42-2.33 (m, 1H), 2.32-2.24 (m, 1H), 1.48 (s, 3H).

### Step 2: methyl 1-[8-methyl-6-[rac-(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexane-4-carboxylate

To a solution of methyl 1-[8-methyl-6-[rac-(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate (180 mg, 350.47 mol, 1 eq) in THF (5 mL) was added dropwise NaH (14.02 mg, 350.47 mol, 60% purity, 1 eq) at 0° C. After addition, the mixture was stirred at this temperature for 1 h, and then SEM-C₁ (87.65 mg, 525.71 mol, 93.04 μL, 1.5 eq) was added dropwise at 0° C. The resulting mixture was stirred at 25° C. for 3 h. LCMS showed starting material was consumed and one main peak with desired mass was detected. The mixture was quenched with H₂O (10 mL) and extracted with EtOAc (15 mL×3), the organic phase was dried, filtered, and concentrated under pressure to give the product. The residue was purified by flash silica gel chromatography (ISCO®; 12 g SepaFlash® Silica Flash Column, Eluent of 0~10% MeOH/DCM gradient) to give compound methyl 1-[8-methyl-6-[rac-(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexane-4-carboxylate (0.9 g, 40% yield) as a white solid.

LC-MS [ESI, M+1]: 644.3.

¹H NMR (400 MHz, CHLOROFORM-d) δ=7.55 (s, 1H), 7.35-7.21 (m, 5H), 6.91 (s, 1H), 5.51 (br d, J=12.0 Hz, 1H), 5.24-5.08 (m, 2H), 4.24 (d, J=5.2 Hz, 1H), 4.09 (s, 1H), 3.75 (d, J=1.2 Hz, 3H), 3.54 (br d, J=8.8 Hz, 1H), 3.50-3.32 (m, 5H), 2.68 (br d, J=7.2 Hz, 1H), 2.56 (s, 3H), 2.40 (br d, J=7.2 Hz, 1H), 2.38-2.26 (m, 4H), 2.26-2.20 (m, 1H), 1.43 (s, 3H), 0.81-0.65 (m, 2H), 0.01-0.17 (m, 9H).

### Step 3: 2-[1-[8-methyl-6-[rac-(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexan-4-yl]propan-2-ol

To a solution of methyl 1-[8-methyl-6-[(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexane-4-carboxylate (40 mg, 62.13 mol, 1 eq) in THF (2 mL) was added MeMgBr (3 M, 20.71 μL, 1 eq) at 0° C. under N₂ atmosphere. The mixture was stirred at 25° C. for 1 h under N₂ atmosphere. LCMS showed starting material was consumed and one main peak with desired mass was detected. The mixture was quenched with H2O (10 mL) and extracted with EtOAc (15 mL×3), the organic phase was dried, filtered, and concentrated under pressure to give the product. The residue was purified by flash silica gel chromatography (ISCO®; 12 g SepaFlash® Silica Flash Column, Eluent of 0~10% MeOH/DCM gradient) to give compound 2-[1-[8-methyl-6-[rac-(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexan-4-yl]propan-2-ol (15 mg, 38% yield) as white solid.

LC-MS [ESI, M+1]: 644.3.

¹H NMR (400 MHz, CHLOROFORM-d) δ=7.73 (d, J=1.6 Hz, 1H), 7.50-7.34 (m, 4H), 7.32 (br s, 1H), 6.99 (s, 1H), 5.36 (s, 2H), 4.20 (d, J=6.4 Hz, 1H), 4.16-4.05 (m, 1H), 3.72-3.52 (m, 6H), 3.47-3.37 (m, 1H), 2.75-2.63 (m, 3H), 2.48 (s, 4H), 2.38-2.15 (m, 4H), 1.54 (d, J=15.2 Hz, 6H), 1.38 (s, 3H), 0.94-0.88 (m, 2H), 0.04-0.09 (m, 9H).

### Step 4: 2-[1-[8-methyl-6-[rac-(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]propan-2-ol

To a solution of 2-[1-[8-methyl-6-[rac-(3R)-3-methyl-3-phenyl-pyrrolidin-1-yl]-[1,2,4]triazolo[1,5-a]pyridin-2-yl]-5-[5-methyl-1-(2-trimethylsilylethoxymethyl)-1,2,4-triazol-3-yl]-2-oxabicyclo[2.1.1]hexan-4-yl]propan-2-ol (15 mg, 23.30 mol, 1 eq) in DCM (1 mL) was added HCl/dioxane (2 M, 116.48 μL, 10 eq). The mixture was stirred at 25° C. for 0.5 h. LCMS showed starting material was consumed and one main peak with desired mass was detected. The mixture was concentrated under pressure to give the product. The residue was purified by prep-HPLC(column: CD11-Phenomenex luna C18 150*25*10 um; mobile phase: [water(FA)-ACN]; gradient:30%-60% B over 8 min) to give compound 2-[1-[8-methyl-6-[rac-(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]propan-2-ol (2.2 mg, 19% yield) as yellow solid.

LC-MS [ESI, M+1]: 514.3.

¹H NMR (400 MHz, CHLOROFORM-d) δ=7.65 (br s, 1H), 7.34 (br dd, J=7.2, 19.2 Hz, 5H), 7.02 (br s, 1H), 4.01 (br d, J=6.5 Hz, 1H), 3.87 (br d, J=6.4 Hz, 1H), 3.61 (br d, J=8.8 Hz, 1H), 3.57-3.47 (m, 2H), 3.42 (br s, 2H), 2.65 (s, 3H), 2.42 (s, 4H), 2.35-2.21 (m, 3H), 1.50 (br d, J=17.6 Hz, 6H), 1.34 (s, 3H).

### Example 4

### (4-amino-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methanol (Compound 4)

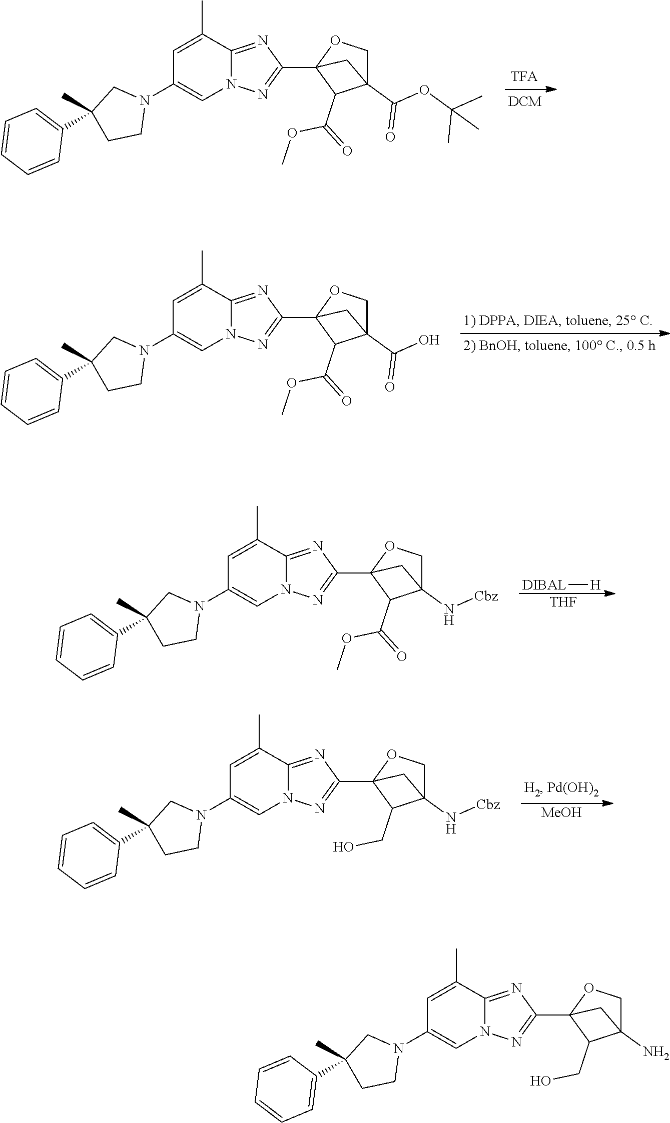

### Step 1: 5-(methoxycarbonyl)-1-(8-methyl-6-((R)-3-methyl-3-phenylpyrrolidin-1-yl)-[1,2,4]triazolo[1,5-a]pyridine-2-yl)-2-oxabicyclo[2.1.1]hexane-4-carboxylic acid

A solution of 4-tert-butyl 5-methyl 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-4,5-dicarboxylate (1 g, 1.88 mmol, 1 eq) in DCM (10 mL), TFA (1 mL) was stirred at 25° C. for 12 h. LCMS showed starting material was consumed and one main peak with desired mass was detected. The mixture was added NaHCO₃ to adjust pH=7, then extracted with ethyl acetate (15 mL×3), the combined organic phase was dried over anhydrous sodium sulfate, filtered and concentrated to give 5-methoxycarbonyl-1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-4-carboxylic acid (900 mg, crude) as yellow solid.

LC-MS [ESI, M+1]: 477.1.

### Step 2: methyl 4-(((benzyloxy)carbonyl)amino)-1-(8-methyl-6-((R)-3-methyl-3-phenylpyrrolidin-1-yl)-[1,2,4]triazolo[1,5-a]pyridine-2-yl)-2-oxabicyclo[2.1.1]hexane-5-carboxylate

To a solution of 5-methoxycarbonyl-1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-4-carboxylic acid (400 mg, 839.41 mol, 1 eq), DIEA (325.46 mg, 2.52 mmol, 438.62 μL, 3 eq) in toluene (8 mL) was added DPPA (346.51 mg, 1.26 mmol, 271.77 μL, 1.5 eq). The mixture was stirred at 25° C. for 1 h. Then BnOH (453.86 mg, 4.20 mmol, 434.73 μL, 5 eq) was added. The mixture was stirred at 100° C. for 1 h. LCMS showed starting material was consumed and one main peak with desired mass was detected. The mixture was quenched with water (10 mL) and extracted with ethyl acetate (10 mL×3), the combined organic phase was dried over anhydrous sodium sulfate, filtered and concentrated to give a residue. The residue was purified by flash silica gel chromatography (ISCO®; 12 g SepaFlash® Silica Flash Column, Eluent of 0~70% Ethylacetate/Petroleum ether gradient) to give methyl 4-(benzyloxycarbonylamino)-1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carboxylate (400 mg, 82% yield) as yellow solid.

LC-MS [ESI, M+1]: 582.3.

¹H NMR (400 MHz, CHLOROFORM-d) δ=7.66 (s, 1H), 7.42-7.29 (m, 8H), 7.24 (br s, 1H), 6.94 (s, 1H), 5.57 (br s, 1H), 5.15 (br s, 2H), 4.23-4.05 (m, 2H), 3.64 (s, 4H), 3.60 (br d, J=8.8 Hz, 1H), 3.55-3.44 (m, 2H), 3.39 (dt, J=3.2, 8.8 Hz, 1H), 2.85-2.74 (m, 1H), 2.63 (s, 3H), 2.44 (br d, J=7.6 Hz, 1H), 2.41-2.32 (m, 1H), 2.29-2.20 (m, 1H), 1.46 (s, 3H).

### Step 3: benzyl (5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)carbamate

To a solution of methyl 4-(benzyloxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (40 mg, 68.77 mol, 1 eq) in THF (1 mL) was added DIBAL-H (1 M, 137.54 μL, 2 eq) at −20° C. under N₂ atmosphere. The mixture was stirred at −20° C. for 0.5 h. LCMS showed starting material was consumed and one main peak with desired mass was detected. The mixture was quenched with water (2 mL) and extracted with ethyl acetate (2 mL×3), the combined organic phase was dried over anhydrous sodium sulfate, filtered and concentrated to give a residue. The residue was purified by prep-HPLC(column: Phenomenex luna C18 150*25 mm*10 um; mobile phase: [water(FA)-ACN]; gradient:48%-78% B over 8 min) to give benzyl N-[5-(hydroxymethyl)-1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-yl]carbamate (5 mg, 13% yield) as white solid.

LC-MS [ESI, M+1]: 554.3.

¹H NMR (400 MHz, CHLOROFORM-d) δ=7.53 (s, 1H), 7.35-7.26 (m, 6H), 7.25-7.19 (m, 3H), 7.16 (br s, 1H), 6.88 (s, 1H), 5.41-5.22 (m, 1H), 5.05 (s, 2H), 4.71-4.51 (m, 1H), 4.06-3.84 (m, 3H), 3.73-3.63 (m, 1H), 3.51 (br d, J=8.8 Hz, 1H), 3.46-3.35 (m, 2H), 3.30 (dt, J=3.6, 8.8 Hz, 1H), 2.71-2.60 (m, 1H), 2.51 (s, 3H), 2.45 (d, J=7.6 Hz, 1H), 2.34-2.24 (m, 2H), 2.21-2.13 (m, 1H), 1.38 (s, 3H).

### Step 4: (4-amino-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methanol

To a solution of benzyl N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate (40 mg, 72.25 mol, 1 eq) in MeOH (1 mL) was added Pd/C (7.69 mg, 7.22 mol, 10% purity, 0.1 eq). The mixture was stirred under H2 balloon atmosphere at 25° C. for 1 h. LCMS showed starting material was consumed and one main peak with desired mass was detected. The mixture was filtered and concentrated in vaco. The residue was purified by prep-HPLC (column: CD07-Daisogel SP-100-8-ODS-PK 150*25*10 um; mobile phase: [water(NH₄HCO3)-ACN]; gradient:24%-54% B over 10 min) to give [4-amino-1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanol (7.24 mg, 24% yield) as white solid.

LC-MS [ESI, M+1]: 420.1.

¹H NMR (400 MHz, METHANOL-d4) δ=7.71 (d, J=1.6 Hz, 1H), 7.41-7.29 (m, 4H), 7.25-7.15 (m, 2H), 3.86-3.76 (m, 1H), 3.73-3.64 (m, 3H), 3.59-3.47 (m, 3H), 3.38 (dt, J=3.6, 8.8 Hz, 1H), 2.55 (s, 3H), 2.40 (dd, J=6.0, 7.2 Hz, 1H), 2.36-2.23 (m, 2H), 2.14 (d, J=6.8 Hz, 2H), 1.42 (s, 3H).

| Cmpd. No. | Synthesis | LC/MS | ¹H NMR |
|---|---|---|---|
| 49 | Similar to | m/z 430.2 | ¹H NMR (400 MHz, CHLOROFORM-d) δ = 7.66 |
| | Example 4 | [M + H]⁺ | (s, 1H), 7.41-7.33 (m, 2H), 7.33-7.29 (m, 2H), |
| | | | 7.27-7.22 (m, 1H), 6.95 (s, 1H), 4.21 (d, J = 6.4 |
| | | | Hz, 1H), 4.17-4.11 (m, 1H), 4.10-4.03 (m, 1H), |
| | | | 3.96 (d, J = 6.4 Hz, 1H), 3.64 (s, 3H), 3.59 (d, J = |
| | | | 8.8 Hz, 1H), 3.55-3.44 (m, 2H), 3.39 (dt, J = 3.6, |
| | | | 8.4 Hz, 1H), 3.34 (s, 1H), 2.64 (s, 3H), 2.37 (td, |
| | | | J = 8.4, 12.0 Hz, 1H), 2.30-2.20 (m, 2H), 2.02 (d, |
| | | | J = 7.6 Hz, 1H), 1.46 (s, 3H) |
| 50 | Similar to | m/z 448.7 | ¹H NMR (400 MHz, METHANOL-d₄) δ = 7.73 |
| | Example 4 | [M + H] | (br s, 1H), 7.40-7.29 (m, 4H), 7.21 (br t, J = 6.4 |
| | | | Hz, 2H), 3.90-3.85 (m, 1H), 3.89-3.75 (m, 2H), |
| | | | 3.67 (br dd, J = 5.6, 11.6 Hz, 1H), 3.61-3.48 (m, |
| | | | 3H), 3.40 (br t, J = 8.4 Hz, 1H), 2.65 (br t, J = 6.4 |
| | | | Hz, 1H), 2.60-2.52 (m, 3H), 2.47 (s, 5H), 2.40- |
| | | | 2.23 (m, 3H), 2.05 (d, J = 7.2 Hz, 1H), 1.44 (s, 3H) |

### Example 5

### 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-amine (Compound 5)

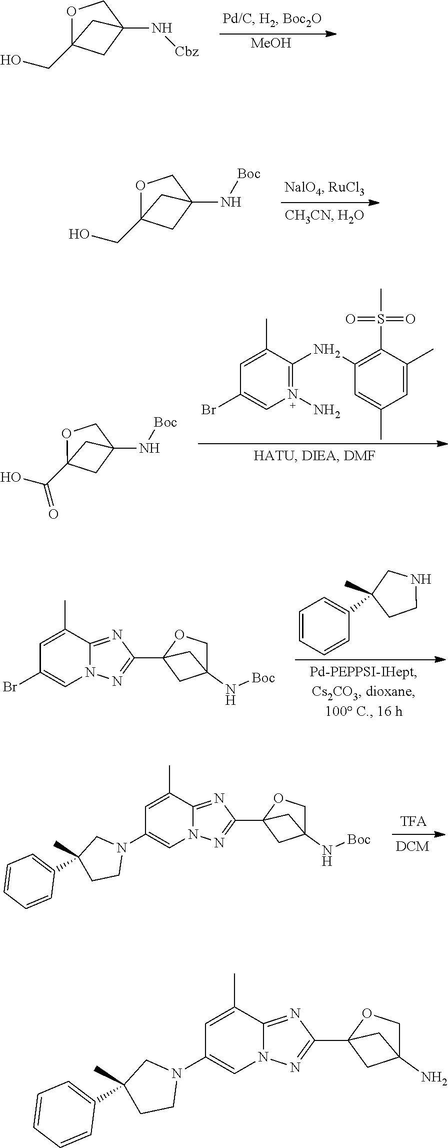

### Step 1: tert-butyl N-[1-(hydroxymethyl)-2-oxabicyclo[2.1.1]hexan-4-yl]carbamate

To a solution of benzyl N-[1-(hydroxymethyl)-2-oxabicyclo[2.1.1]hexan-4-yl]carbamate (10 g, 37.98 mmol, 1 eq) and Boc₂O (24.87 g, 113.94 mmol, 26.18 mL, 3 eq) in EtOH (200 mL) was added Pd/C (2.02 g, 1.90 mmol, 10% purity, 0.05 eq) under H2 atmosphere. The mixture was stirred under H2 (76.56 mg, 37.98 mmol, 1 eq) (50 Psi) at 25° C. for 12 h. LCMS showed starting material was consumed and one main peak with desired mass was detected. The residue was purified by column chromatography (SiO₂, PE:EtOAc=3:1, Rf=0.1) to give compound tert-butyl N-[1-(hydroxymethyl)-2-oxabicyclo[2.1.1]hexan-4-yl]carbamate (7.3 g, 84% yield) was obtained as a white solid. [3026]¹H NMR (400 MHz, CHLOROFORM-d) δ=5.13 (br s, 1H), 3.83 (br d, J=5.6 Hz, 2H), 3.80 (br s, 2H), 2.26-2.05 (m, 1H), 1.95 (br s, 3H), 1.45 (s, 9H).

### Step 2: 4-(tert-butoxycarbonylamino)-2-oxabicyclo[2.1.1]hexane-1-carboxylic acid

To a solution of tert-butyl N-[1-(hydroxymethyl)-2-oxabicyclo[2.1.1]hexan-4-yl]carbamate (3 g, 13.08 mmol, 1 eq), RuCl₃ (81.43 mg, 392.55 mol, 26.18 μL, 0.03 eq), NaOH (2.09 g, 52.34 mmol, 4 eq) in CH₃CN (18 mL), H₂O (30 mL), H₂O (30 mL) and DCM (18 mL) was added NaIO₄ (13.99 g, 65.42 mmol, 3.63 mL, 5 eq) at 0° C. The mixture was stirred at 25° C. for 1 h. LCMS showed starting material was consumed and one main peak with desired mass was detected. The reaction was quenched by water (20 mL) slowly and then the mixture was filtered and washed with water. An aqueous layer was washed with MTBE (10 mL×3). The aqueous layer was acidified with 5M HCl to pH=2 and then extracted with EtOAc (10 mL×3). The combined organic phases were dried over Na₂SO₄, filtered and concentrated in vacuum. The residue was purified by column chromatography (SiO₂, DCM:MeOH=10:1, Rf=0.3) to give compound 4-(tert-butoxycarbonylamino)-2-oxabicyclo[2.1.1]hexane-1-carboxylic acid (1.8 g, 57% yield) was obtained as a black solid

¹H NMR (400 MHz, DMSO-d6) δ=7.71 (br s, 1H), 3.66 (br s, 2H), 2.27 (br s, 2H), 1.85 (br d, J=1.6 Hz, 2H), 1.38 (s, 9H).

### Step 3: tert-butyl N-[1-(6-bromo-8-methyl-[1,2,4]triazolo[1,5-a]pyridine-2-yl)-2-oxabicyclo[2.1.1]hexan-4-yl]carbamate

To a solution of 4-(tert-butoxycarbonylamino)-2-oxabicyclo[2.1.1]hexane-1-carboxylic acid (1.5 g, 6.17 mmol, 1 eq) and 5-bromo-3-methyl-pyridin-1-ium-1,2-diamine;2,4,6-trimethylbenzenesulfonate (4.96 g, 12.33 mmol, 2 eq) in DMF (20 mL) was added HATU (3.52 g, 9.25 mmol, 1.5 eq). The mixture was stirred at 25° C. for 0.5 h, and then DIEA (3.98 g, 30.83 mmol, 5.37 mL, 5 eq) was added at 25° C. The mixture was stirred at 25° C. for 1 h. LCMS showed starting material was consumed and one main peak with desired mass was detected. The reaction was added water (10 mL) and then extracted with EtOAc (10 mL×3). The combined organic phase was washed with brine (5 mL), dried with anhydrous Na₂SO₄, filtered and concentrated in vacuum. The residue was purified by column chromatography (SiO2, PE:EtOAc=1:1, Rf=0.2) to give compound tert-butyl N-[1-(6-bromo-8-methyl-[1,2,4]triazolo[1,5-a]pyridine-2-yl)-2-oxabicyclo[2.1.1]hexan-4-yl]carbamate (1.8 g, 71% yield) was obtained as a brown oil.

LC-MS [ESI, M+1]: 409.0.

### Step 4: tert-butyl N-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-yl]carbamate

To a solution of tert-butyl N-[1-(6-bromo-8-methyl-[1,2,4]triazolo[1,5-a]pyridine-2-yl)-2-oxabicyclo[2.1.1]hexan-4-yl]carbamate (1 g, 2.44 mmol, 1 eq) and (3R)-3-methyl-3-phenyl-pyrrolidine (393.97 mg, 2.44 mmol, 1 eq) in dioxane (12 mL) was added 1,3-bis[2,6-bis(1-propylbutyl)phenyl]-4,5-dichloro-2H-imidazol-1-ium-2-ide;3-chloropyridine;dichloropalladium (118.84 mg, 122.17 mol, 0.05 eq) and Cs₂CO₃ (2.39 g, 7.33 mmol, 3 eq). The mixture was stirred at 100° C. for 12 h under N₂. LCMS showed starting material was consumed and one main peak with desired mass was detected. The reaction was added water (10 mL) and then extracted with EtOAc (10 mL×3). The combined organic phase was washed with brine (10 mL), dried with anhydrous Na₂SO₄, filtered and concentrated in vacuum. The residue was purified by column chromatography (SiO₂, PE:EtOAc=0:1, Rf=0.4) to give compound tert-butyl N-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-yl]carbamate (660 mg, 55% yield) was obtained as a yellow gum. [3035]¹H NMR (400 MHz, CHLOROFORM-d) δ=7.63 (s, 1H), 7.40-7.34 (m, 2H), 7.33-7.28 (m, 2H), 7.27-7.21 (m, 1H), 6.93 (s, 1H), 5.22-5.04 (m, 1H), 4.04 (br s, 2H), 3.58 (d, J=8.8 Hz, 1H), 3.54-3.43 (m, 2H), 3.37 (dt, J=3.2, 8.4 Hz, 1H), 2.62 (s, 3H), 2.53 (br s, 2H), 2.45-2.32 (m, 3H), 2.29-2.20 (m, 1H), 1.51-1.43 (m, 12H).

### Step 5: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine

To a solution of tert-butyl N-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-yl]carbamate (100 mg, 204.25 mol, 1 eq) in DCM (2 mL) was added TFA (1.54 g, 13.46 mmol, 1 mL, 65.91 eq). The mixture was stirred at 25° C. for 1 h. LCMS showed starting material was consumed and one main peak with desired mass was detected. The reaction mixture was concentrated under the reduced pressure to give the product. The pH was adjusted to around 7 by progressively aq. NaHCO₃, and then extracted with EtOAc (5 mL×3). The combined organic layers were washed with brine (5 mL), dried over Na₂SO₄, filtered and concentrated under reduced pressure to give compound 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-4-amine (66 mg, 83% yield) was obtained as a yellow gum.

LC-MS [ESI, M+1]: 390.1.

¹H NMR (400 MHz, CHLOROFORM-d) δ=7.63 (d, J=1.6 Hz, 1H), 7.40-7.33 (m, 2H), 7.33-7.29 (m, 2H), 7.27-7.21 (m, 1H), 6.93 (s, 1H), 3.83 (s, 2H), 3.58 (d, J=8.8 Hz, 1H), 3.54-3.43 (m, 2H), 3.37 (dt, J=3.6, 8.8 Hz, 1H), 2.63 (s, 3H), 2.41-2.21 (m, 6H), 1.46 (s, 3H).

| Cmpd. No. | Synthesis | LC/MS | ¹H NMR |
|---|---|---|---|
| 51 | Similar to | m/z 404.2 | ¹H NMR (400 MHz, CHLOROFORM-d) δ = 7.67 |
| | Example 5 | [M + H]⁺ | (s, 1H), 7.43-7.33 (m, 2H), 7.32-7.29 (m, 1H), |
| | | | 7.26 (br d, J = 7.6 Hz, 2H), 6.96 (s, 1H), 3.86 (s, |
| | | | 2H), 3.66-3.59 (m, 1H), 3.54 (br d, J = 8.8 Hz, |
| | | | 1H), 3.50-3.41 (m, 1H), 3.38-3.28 (m, 1H), |
| | | | 2.67 (s, 3H), 2.45-2.23 (m, 6H), 1.89 (br dd, J = |
| | | | 7.6, 14.5 Hz, 2H), 0.83-0.65 (m, 3H) |
| 52 | Similar to | m/z 432.2 | ¹H NMR (400 MHz, CHLOROFORM-d) δ = 7.63 |
| | Example 5 | [M + H]⁺ | (s, 1H), 7.41-7.28 (m, 4H), 7.27-7.21 (m, 1H), |
| | | | 6.93 (s, 1H), 6.07 (br s, 1H), 4.10 (s, 2H), 3.58 (d, |
| | | | J = 8.8 Hz, 1H), 3.54-3.43 (m, 2H), 3.38 (dt, J = |
| | | | 3.6, 8.8 Hz, 1H), 2.63 (s, 3H), 2.60-2.55 (m, 2H), |
| | | | 2.49 (br dd, J = 1.2, 4.4 Hz, 2H), 2.41-2.32 (m, |
| | | | 1H), 2.29-2.20 (m, 1H), 2.03 (s, 3H), 1.46 (s, 3H) |
| 53 | Similar to | m/z 490.3 | ¹H NMR (400 MHz, CHLOROFORM-d) δ = 7.64 |
| | Example 5 | [M + H]⁺ | (s, 1H), 7.42-7.33 (m, 2H), 7.33-7.28 (m, 2H), |
| | | | 7.27-7.22 (m, 1H), 6.97 (s, 1H), 6.81 (br s, 1H), |
| | | | 4.08 (s, 2H), 3.59 (br d, J = 8.8 Hz, 1H), 3.54- |
| | | | 3.44 (m, 2H), 3.38 (dt, J = 3.2, 8.8 Hz, 1H), 2.65- |
| | | | 2.56 (m, 5H), 2.48 (br d, J = 3.6 Hz, 2H), 2.42- |
| | | | 2.34 (m, 3H), 2.28-2.24 (m, 1H), 1.46 (s, 3H), |
| | | | 1.31 (s, 6H) |
| 54 | Similar to | m/z 446.2 | ¹H NMR (400 MHz, CHLOROFORM-d) δ = 7.64 |
| | Example 5 | [M + H]⁺ | (s, 1H), 7.41-7.34 (m, 2H), 7.33-7.29 (m, 2H), |
| | | | 7.24 (br s, 1H), 6.98 (s, 1H), 5.70 (br s, 1H), 4.15 |
| | | | (s, 2H), 3.60 (d, J = 8.8 Hz, 1H), 3.55-3.45 (m, |
| | | | 2H), 3.43-3.34 (m, 3H), 2.64 (s, 3H), 2.60-2.54 |
| | | | (m, 2H), 2.42-2.33 (m, 3H), 2.29-2.23 (m, 1H), |
| | | | 1.46 (s, 3H), 1.20 (t, J = 7.3 Hz, 3H) |
| 55 | Similar to | m/z 418.0 | ¹H NMR (400 MHz, CHLOROFORM-d) δ = |
| | Example 5 | [M + H]⁺ | 7.64 (s, 1H), 7.43-7.34 (m, 2H), 7.34-7.28 (m, |
| | | | 2H), 7.24 (br s, 1H), 6.99 (s, 1H), 5.86-5.65 (m, |
| | | | 1H), 5.64-5.43 (m, 1H), 4.19 (s, 2H), 3.60 (d, J = |
| | | | 8.8 Hz, 1H), 3.56-3.44 (m, 2H), 3.39 (dt, J = |
| | | | 3.2, 8.7 Hz, 1H), 2.64 (s, 5H), 2.46-2.32 (m, |
| | | | 3H), 2.31-2.22 (m, 1H), 1.47 (s, 3H) |
| 56 | Similar to | m/z 485.2 | ¹H NMR (400 MHz, CHLOROFORM-d) δ = 7.62 |
| | Example 5 | [M + H]⁺ | (br s, 1H), 7.35 (br d, J = 6.8 Hz, 2H), 7.30 (br d, |
| | | | J = 7.2 Hz, 2H), 7.26-7.21 (m, 1H), 6.94 (br s, |
| | | | 1H), 4.13 (br s, 2H), 3.89 (br s, 2H), 3.60-3.36 |
| | | | (m, 4H), 2.60 (br s, 3H), 2.46 (br s, 3H), 2.37 (br |
| | | | s, 2H), 2.25 (br d, J = 2.8 Hz, 2H), 2.17 (br s, 2H), |
| | | | 1.46 (br s, 3H) |
| 57 | Similar to | m/z 377.3 | ¹H NMR (400 MHz, CHLOROFORM-d) δ = 7.88 |
| | Example 5 | [M + H]⁺ | (s, 1H), 7.42-7.36 (m, 2H), 7.32 (t, J = 7.2 Hz, |
| | | | 2H), 7.26-7.20 (m, 1H), 7.04 (s, 1H), 3.99 (s, |
| | | | 2H), 3.82 (s, 2H), 2.58 (s, 3H), 2.27 (s, 4H), 1.04 |
| | | | (br d, J = 4.0 Hz, 4H) |
| 58 | Similar to | m/z 404.2 | ¹H NMR (400 MHz, METHANOL-d4) δ = 7.72 |
| | Example 5 | [M + H]⁺ | (s, 1H), 7.41-7.30 (m, 4H), 7.25-7.17 (m, 2H), |
| | | | 3.88 (s, 2H), 3.60-3.51 (m, 3H), 3.40 (dt, J = 3.6, |
| | | | 8.8 Hz, 1H), 3.10 (s, 2H), 2.58 (s, 3H), 2.38-2.22 |
| | | | (m, 4H), 2.01 (br d, J = 4.8 Hz, 2H), 1.44 (s, 3H) |
| 59 | Similar to | m/z 406.1 | ¹H NMR (400 MHz, CHLOROFORM-d) δ = 7.67 |
| | Example 5 | [M + H]⁺ | (s, 1H), 7.41-7.33 (m, 2H), 7.33-7.28 (m, 2H), |
| | | | 7.27-7.22 (m, 1H), 7.15 (s, 1H), 5.03 (s, 2H), |
| | | | 3.83 (s, 2H), 3.61 (br d, J = 8.8 Hz, 1H), 3.57- |
| | | | 3.47 (m, 2H), 3.47-3.33 (m, 2H), 2.41-2.34 (m, |
| | | | 1H), 2.26 (dt, J = 4.8, 7.6 Hz, 5H), 1.91 (br s, 2H), |
| | | | 1.46 (s, 3H) |
| 60 | Similar to | m/z 405.2 | ¹H NMR (400 MHz, CHLOROFORM-d) δ = |
| | Example 5 | [M + H]⁺ | 7.59-7.54 (m, 1H), 7.31-7.17 (m, 5H), 6.89- |
| | | | 6.83 (m, 1H), 3.98-3.92 (m, 2H), 3.91-3.85 |
| | | | (m, 2H), 3.55-3.48 (m, 1H), 3.47-3.36 (m, |
| | | | 2H), 3.34-3.25 (m, 1H), 2.56 (s, 3H), 2.33- |
| | | | 2.22 (m, 3H), 2.20-2.13 (m, 1H), 2.06-2.00 |
| | | | (m, 2H), 1.41-1.36 (m, 3H) |
| 61 | Similar to | m/z 365.0 | ¹H NMR (400 MHz, CHLOROFORM-d) δ = 7.93 |
| | Example 5 | [M + H]⁺ | (s, 1H), 7.40-7.31 (m, 2H), 7.30-7.23 (m, 3H), |
| | | | 7.04 (s, 1H), 4.08-4.00 (m, 1H), 3.99-3.92 (m, |
| | | | 1H), 3.82 (s, 2H), 3.38-3.14 (m, 1H), 2.59 (s, |
| | | | 3H), 2.27 (s, 4H), 1.42 (d, J = 6.8 Hz, 3H) |
| 62 | Similar to | m/z 513.0 | ¹H NMR (400 MHz, CHLOROFORM-d) δ = |
| | Example 5 | [M + H]⁺ | 7.91 (s, 1H), 7.62 (d, J = 1.6 Hz, 1H), 7.40-7.33 |
| | | | (m, 2H), 7.33-7.28 (m, 2H), 7.27-7.22 (m, |
| | | | 1H), 7.16 (s, 1H), 6.93 (s, 1H), 4.78 (s, 2H), 4.07 |
| | | | (s, 2H), 3.58 (d, J = 8.8 Hz, 1H), 3.53-3.43 (m, |
| | | | 2H), 3.37 (dt, J = 3.6, 8.8 Hz, 1H), 2.65-2.57 |
| | | | (m, 5H), 2.52 (s, 3H), 2.47 (dd, J = 1.6, 4.4 Hz, |
| | | | 2H), 2.36 (td, J = 8.4, 12.4 Hz, 1H), 2.28-2.20 |
| | | | (m, 1H), 1.45 (s, 3H) |
| 63 | Similar to | m/z 500.2 | ¹H NMR (400 MHz, CHLOROFORM-d) δ = |
| | Example 5 | [M + H]⁺ | 7.63 (br s, 1H), 7.35 (br dd, J = 2.8, 7.2 Hz, 2H), |
| | | | 7.32-7.28 (m, 2H), 7.26 (br s, 1H), 7.25-7.22 |
| | | | (m, 1H), 6.94 (br s, 1H), 3.88 (br s, 2H), 3.62- |
| | | | 3.54 (m, 1H), 3.53-3.43 (m, 2H), 3.41-3.34 |
| | | | (m, 1H), 3.28-3.10 (m, 2H), 3.01-2.86 (m, |
| | | | 2H), 2.63 (br d, J = 2.8 Hz, 3H), 2.44-2.33 (m, |
| | | | 3H), 2.24 (br dd, J = 4.0, 11.6 Hz, 2H), 2.15 (br |
| | | | d, J = 12.0 Hz, 4H), 1.46 (br d, J = 3.2 Hz, 3H) |
| 64 | Similar to | m/z 418.2 | |
| | Example 5 | [M + H]⁺ | |
| 65 | Similar to | m/z 432.3 | |
| | Example 5 | [M + H]⁺ | |
| 66 | Similar to | m/z 404.3 | |
| | Example 5 | [M + H]⁺ | |
| 67 | Similar to | m/z 404.2 | |
| | Example 5 | [M + H]⁺ | |
| 68 | Similar to | m/z 404.3 | |
| | Example 5 | [M + H]⁺ | |

### Example 6

### Compound 1: 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazole-3-carboxamide (Compound 6)

### Step 1: dimethyl (E)-2-allyloxybut-2-enedioate

To a solution of dimethyl but-2-ynedioate (50 g, 351.84 mmol, 1 eq) in DCM (200 mL) was added prop-2-en-1-ol (21.46 g, 369.43 mmol, 25.12 mL, 1.05 eq). Then the solution was dropwise added DABCO (3.95 g, 35.18 mmol, 3.87 mL, 0.1 eq) in DCM (30 mL). The mixture was stirred at 25° C. for 3 h. TLC (PE:EtOAc=5:1) showed starting material was not consumed completely and many new spots formed. The reaction was concentrated under reduced pressure to give a residue. The residue was purified by flash silica gel chromatography (ISCO®; 330 g SepaFlash® Silica Flash Column, Eluent of 0~5% EtOAc:PE @150 mL/min) to give compound dimethyl (E)-2-allyloxybut-2-enedioate (130 g, 46% yield) as a colourless liquid.

¹H NMR (400 MHz, CHLOROFORM-d) δ=5.97-5.86 (m, 1H), 5.41-5.26 (m, 2H), 5.17 (s, 1H), 4.39 (d, J=5.2 Hz, 2H), 3.85 (s, 3H), 3.66 (s, 3H).

### Step 2: dimethyl 2-oxabicyclo[2.1.1]hexane-1,5-dicarboxylate

To a solution of dimethyl (E)-2-allyloxybut-2-enedioate (120 g, 599.44 mmol, 1 eq) in ACN (1000 mL) was added diphenylmethanone (10.92 g, 59.94 mmol, 0.1 eq). The mixture was stirred at 25° C. (366 nm, in flow) for 1 h. TLC (PE:EtOAc=5:1) showed starting material was not consumed completely and three new spots formed. The reaction mixture was concentrated in vacuum to give compound dimethyl 2-oxabicyclo[2.1.1]hexane-1,5-dicarboxylate (120 g, crude). as a yellow liquid.

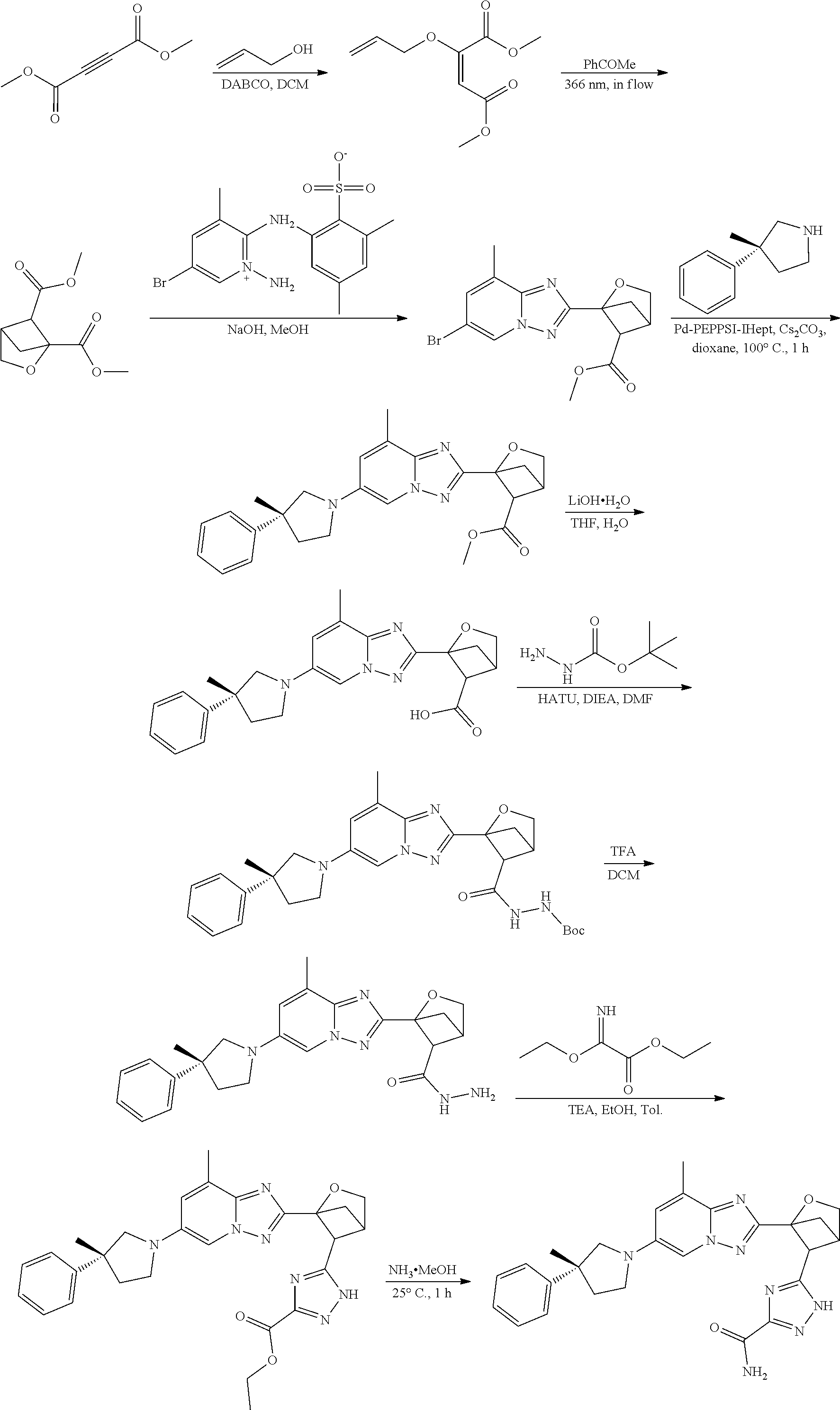

### Step 3: methyl 1-(6-bromo-8-methyl-[1,2,4]triazolo[1,5-a]pyridine-2-yl)-2-oxabicyclo[2.1.1]hexane-5-carboxylate

To a solution of dimethyl 2-oxabicyclo[2.1.1]hexane-1,5-dicarboxylate (37.82 g, 188.91 mmol, 2 eq) and 5-bromo-3-methyl-pyridin-1-ium-1,2-diamine;2,4,6-trimethylbenzenesulfonate (38 g, 94.46 mmol, 1 eq) in MeOH (500 mL) was added NaOH (5.67 g, 141.68 mmol, 1.5 eq). The mixture was stirred at 70° C. for 1 h. LCMS showed starting material was consumed completely and the desired compound was detected. The reaction was concentrated under reduced pressure to give a residue. The residue was purified by flash silica gel chromatography (ISCO®; 200 g SepaFlash® Silica Flash Column, Eluent of 30~37% EtOAc:PE @100 mL/min). to give compound methyl 1-(6-bromo-8-methyl-[1,2,4]triazolo[1,5-a]pyridine-2-yl)-2-oxabicyclo[2.1.1]hexane-5-carboxylate (8 g, 24% yield) as a yellow solid.

LC-MS [ESI, M+1]: 353.9.

¹H NMR (400 MHz, DMSO-d6) δ=9.18 (d, J=0.4 Hz, 1H), 7.68 (s, 1H), 4.02-3.98 (m, 1H), 3.86 (d, J=6.4 Hz, 1H), 3.50 (s, 3H), 3.32 (s, 1H), 3.23 (t, J=2.8 Hz, 1H), 2.53 (s, 3H), 2.19 (dd, J=7.2, 2.8 Hz, 1H), 1.70 (d, J=7.2 Hz, 1H).

### Step 4: methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

To a solution of methyl 1-(6-bromo-8-methyl-[1,2,4]triazolo[1,5-a]pyridin-2-yl)-2-oxabicyclo[2.1.1]hexane-5-carboxylate (5 g, 14.20 mmol, 1 eq) and (3R)-3-methyl-3-phenyl-pyrrolidine (2.29 g, 14.20 mmol, 1 eq) in dioxane (80 mL) was added Pd-PEPPSI-IheptCl (690.53 mg, 709.86 mol, 0.05 eq) and Cs₂CO₃ (13.88 g, 42.59 mmol, 3 eq). The mixture was stirred at 100° C. for 1 h under N₂. LCMS showed starting material was consumed completely and the desired compound was detected. The reaction was concentrated under reduced pressure to give a residue. The residue was purified by flash silica gel chromatography (ISCO®; 40 g SepaFlash® Silica Flash Column, Eluent of 55~70% EtOAc:PE @80 mL/min) to give compound methyl 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carboxylate (4.7 g, 76% yield) as a brown solid.

LC-MS [ESI, M+1]: 433.3.

¹H NMR (400 MHz, CHLOROFORM-d) δ=7.66 (s, 1H), 7.40-7.33 (m, 2H), 7.33-7.28 (m, 2H), 7.27-7.22 (m, 1H), 6.96-6.91 (m, 1H), 4.27 (d, J=6.4 Hz, 1H), 4.02 (d, J=6.4 Hz, 1H), 3.63-3.55 (m, 4H), 3.54-3.44 (m, 2H), 3.38 (dt, J=8.8, 3.6 Hz, 1H), 3.34-3.26 (m, 2H), 2.67-2.60 (m, 3H), 2.40-2.32 (m, 1H), 2.30-2.20 (m, 2H), 1.90 (d, J=8.0 Hz, 1H), 1.46 (s, 3H).

### Step 5:1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid

To a solution of methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (1.50 g, 3.47 mmol, 1 eq) in THF (15 mL) was added LiOH·H₂O (436.56 mg, 10.40 mmol, 3 eq) and H₂O (2 mL). The mixture was stirred at 25° C. for 1 h. LCMS showed starting material was consumed completely and the desired compound was detected. The reaction was added HCl (2 M) until pH around 3 and then extracted with EtOAc (30 mL×5). The combined organic phase was washed with brine (20 mL×3), dried over anhydrous Na₂SO₄, filtered and concentrated in vacuum to give compound 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carboxylic acid (1.4 g, 96% yield) as a yellow oil. LC-MS [ESI, M+1]: 419.2.

### Step 6: tert-butyl N-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]carbamate

To a solution of 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carboxylic acid (900.00 mg, 2.15 mmol, 1 eq) and tert-butyl N-aminocarbamate (341.07 mg, 2.58 mmol, 1.2 eq) in DMF (12 mL) was added HATU (1.23 g, 3.23 mmol, 1.5 eq) and DIEA (833.85 mg, 6.45 mmol, 1.12 mL, 3 eq). The mixture was stirred at 25° C. for 1 h. LCMS showed starting material was consumed completely and the desired compound was detected. The reaction was added H₂O (10 mL) and then extracted with EtOAc (30 mL×3). The combined organic phase was washed with brine (20 mL×3), dried over anhydrous Na₂SO₄, filtered and concentrated in vacuum. The residue was purified by flash silica gel chromatography (ISCO®; 20 g SepaFlash® Silica Flash Column, Eluent of 60~80% EtOAc:PE @80 mL/min). to give compound tert-butyl N-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]carbamate (950 mg, 83% yield) as a yellow oil.

LC-MS [ESI, M+1]: 533.3.

¹H NMR (400 MHz, CHLOROFORM-d) δ=10.73 (d, J=7.2 Hz, 1H), 7.63 (s, 1H), 7.40-7.34 (m, 2H), 7.34-7.29 (m, 2H), 7.25 (s, 1H), 6.98 (s, 1H), 6.68-6.35 (m, 1H), 4.17 (d, J=6.4 Hz, 1H), 4.01 (d, J=6.4 Hz, 1H), 3.60 (d, J=8.8 Hz, 1H), 3.55-3.46 (m, 2H), 3.45-3.35 (m, 2H), 3.10 (s, 1H), 2.62 (s, 3H), 2.42-2.33 (m, 1H), 2.29-2.22 (m, 1H), 2.18 (dd, J=7.6, 2.8 Hz, 1H), 2.08 (s, 1H), 1.50-1.46 (m, 12H).

### Step 7: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbohydrazide

To a solution of tert-butyl N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]carbamate (950 mg, 1.78 mmol, 1 eq) in DCM (6 mL) was added HCl/dioxane (2 M, 3.39 mL, 3.80 eq). The mixture was stirred at 25° C. for 0.5 h. LCMS showed starting material was consumed completely and the desired compound was detected. The reaction mixture was concentrated in vacuum. The reaction was added NaHCO₃ until pH around 7 and then extracted with DCM (15 mL×5). The combined organic phase was washed with brine (10 mL×3), dried over anhydrous Na₂SO₄, filtered and concentrated in vacuum to give compound 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbohydrazide (715 mg, 92% yield) as a yellow oil.

LC-MS [ESI, M+1]: 433.2.

### Step 8: ethyl 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazole-3-carboxylate

To a solution of 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbohydrazide (600 mg, 1.39 mmol, 1 eq) and ethyl 2-ethoxy-2-imino-acetate (503.89 mg, 3.47 mmol, 2.50 eq) in EtOH (6 mL) was added TEA (210.56 mg, 2.08 mmol, 289.63 μL, 1.5 eq). The mixture was stirred at 80° C. for 1 h and then the solution was added toluene (5 mL). The mixture was stirred at 145° C. for 12 h. LCMS showed starting material was consumed completely and the desired compound was detected. The mixture was filtered and concentrated under pressure to give the product. The residue was purified by prep-TLC (SiO₂, DCM:MeOH=10:1, Rf=0.45) and the eluted with DCM:MeOH=10:1 (150 mL) to give compound ethyl 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazole-3-carboxylate (80 mg, 11% yield) as a brown solid.

LC-MS [ESI, M+1]: 514.3.

### Step 9: 5-[1-[8-methyl-6-[(3R)-3-methyl-3-phenyl-pyrrolidin-1-yl]-[1,2,4]triazolo[1,5-a pyridine-2-yl]-2-oxabicyclo[2.1.1]hexan-5-yl]-1H-1,2,4-triazole-3-carboxamide

To a solution of ethyl 5-[1-[8-methyl-6-[(3R)-3-methyl-3-phenyl-pyrrolidin-1-yl]-[1,2,4]triazolo[1,5-a pyridine-2-yl]-2-oxabicyclo[2.1.1]hexan-5-yl]-1H-1,2,4-triazole-3-carboxylate (20 mg, 38.94 mol, 1 eq) in MeOH (0.5 mL) was added NH₃·MeOH (7 M, 0.5 mL, 89.88 eq). The mixture was stirred at 25° C. for 1 h. LCMS showed starting material was consumed completely and the desired compound was detected. The mixture was filtered and concentrated under pressure to give the product. The residue was purified by prep-HPLC (column: CD02-Waters Xbidge BEH C18 150*25*10 um; mobile phase: [water(NH₃H₂O)-ACN]; gradient:28%-58% B over 13 min) to give compound 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazole-3-carboxamide (3.28 mg, 17% yield) as yellow solid.

LC-MS [ESI, M+1]: 485.3.

¹H NMR (400 MHz, CHLOROFORM-d) δ=7.69 (s, 1H), 7.43-7.34 (i, 2H), 7.34-7.30 (m, 2H), 7.27-7.23 (7, 1H), 7.07 (s, 1H), 6.80 (s, 1H), 5.51 (s, 1H), 4.62-4.48 (m, 1H), 4.08 (d, J=9.2 Hz, 1H), 3.66-3.60 (m, 1H), 3.57-3.47 (m, 2H), 3.45-3.33 (d, 2H), 3.30-3.13 (i, 3H), 2.74 (d, J=5.2 Hz, 1H), 2.70 (s, 3H), 2.40 (dd, J=8.4, 4.0 Hz, 1H), 2.30-2.27 (11, 1H), 1.48 (s, 3H).

| Cmpd. No. | Synthesis | LC/MS | ¹H NMR |
|---|---|---|---|
| 69 | Similar to | m/z 418.3 | ¹H NMR (400 MHz, CHLOROFORM-d) δ = 8.31 (br |
| | Example 6 | [M + H]⁺ | d, J = 2.0 Hz, 1H), 7.63 (s, 1H), 7.40-7.34 (m, 2H), |
| | | | 7.33-7.27 (m, 3H), 7.00 (s, 1H), 5.61 (br s, 1H), 4.93 |
| | | | (s, 1H), 4.20 (d, J = 6.0 Hz, 1H), 4.02 (d, J = 6.0 Hz, |
| | | | 1H), 3.61 (d, J = 8.8 Hz, 1H), 3.56-3.45 (m, 2H), |
| | | | 3.40 (dt, J = 3.6, 8.8 Hz, 1H), 3.11 (s, 1H), 2.60 (s, |
| | | | 3H), 2.39 (td, J = 8.2, 12.2 Hz, 1H), 2.31-2.24 (m, |
| | | | 1H), 2.21 (d, J = 7.8 Hz, 1H), 2.12 (d, J = 7.8 Hz, 1H), |
| | | | 1.49-1.44 (m, 3H) |
| 70 | Similar to | m/z 455.2 | ¹H NMR (400 MHz, CHLOROFORM-d) δ = 7.65 (d, |
| | Example 6 | [M + H]⁺ | J = 1.2 Hz, 1H), 7.41-7.34 (m, 2H), 7.34-7.29 (m, |
| | | | 2H), 7.27-7.23 (m, 1H), 7.00 (s, 1H), 6.71 (s, 1H), |
| | | | 3.98 (d, J = 6.4 Hz, 1H), 3.87 (d, J = 6.4 Hz, 1H), 3.63- |
| | | | 3.56 (m, 2H), 3.56-3.45 (m, 3H), 3.40 (dt, J = 8.4, |
| | | | 3.6 Hz, 1H), 2.64 (s, 3H), 2.44-2.35 (m, 2H), 2.32- |
| | | | 2.22 (m, 4H), 2.13 (d, J = 8.0 Hz, 1H), 1.47 (s, 3H). |
| 71 | Similar to | m/z 490.4 | ¹H NMR (DMSO-d₆, 400 MHz): δ = 7.88 (d, J = 1.6 |
| | Example 6 | [M + H]⁺ | Hz, 0.9H), 7.32-7.39 (m, 4.0H), 7.22 (s, 2.0H), 4.54 |
| | | | (br s, 0.9H), 4.08 (d, J = 5.6 Hz, 1.0H), 3.74 (d, J = 5.6 |
| | | | Hz, 1.0H), 3.56 (s, 1.1H), 3.50 (br d, J = 9.2 Hz, |
| | | | 3.1H), 2.99 (t, J = 3.2 Hz, 1.0H), 2.24 (t, J = 6.8 Hz, |
| | | | 2.1H), 2.15 (d, J = 1.6 Hz, 1.0H), 1.91 (dd, J = 6.8, 2.8 |
| | | | Hz, 1.0H), 1.63 (dd, J = 7.2, 1.4 Hz, 1.0H), 1.34 (s, |
| | | | 3.2H), 1.17 (s, 3.0H), 1.08 ppm (s, 3.0H) |
| 72 | Similar to | m/z 432.2 | ¹H NMR (CHLOROFORM-d, 400 MHz): δ = 8.45 |
| | Example 6 | [M + H]⁺ | (br s, 0.9H), 7.65 (s, 1.0H), 7.38 (d, J = 7.2 Hz, 1.9H), |
| | | | 7.30-7.35 (m, 1.9H), 7.25-7.28 (m, 0.9H), 7.01 (s, |
| | | | 1.0H), 4.09 (d, J = 6.4 Hz, 1.0H), 4.00 (d, J = 6.2 Hz, |
| | | | 0.9H), 3.62 (d, J = 8.8 Hz, 1.0H), 3.47-3.58 (m, |
| | | | 2.0H), 3.37-3.45 (m, 1.9H), 3.04 (br d, J = 2.8 Hz, |
| | | | 1.0H), 2.88 (d, J = 4.4 Hz, 3.0H), 2.63 (s, 3.0H), 2.40 |
| | | | (dt, J = 12.4, 8.3 Hz, 1.1H), 2.23-2.32 (m, 1.1H), 2.18 |
| | | | (br dd, J = 7.6, 2.6 Hz, 1.1H), 2.03 (d, J = 7.6 Hz, |
| | | | 1.1H), 1.49 ppm (s, 3.0H) |
| 73 | Similar to | m/z 433.4 | ¹H NMR (DMSO-d₆, 400 MHz): δ = 7.88 (d, J = 1.6 |
| | Example 6 | [M + H]⁺ | Hz, 0.9H), 7.32-7.39 (m, 4.0H), 7.22 (s, 2.0H), 4.54 |
| | | | (br s, 0.9H), 4.08 (d, J = 5.6 Hz, 1.0H), 3.74 (d, J = 5.6 |
| | | | Hz, 1.0H), 3.56 (s, 1.1H), 3.50 (br d, J = 9.2 Hz, |
| | | | 3.1H), 2.99 (t, J = 3.2 Hz, 1.0H), 2.24 (t, J = 6.8 Hz, |
| | | | 2.1H), 2.15 (d, J = 1.6 Hz, 1.0H), 1.91 (dd, J = 6.8, 2.8 |
| | | | Hz, 1.0H), 1.63 (dd, J = 7.2, 1.4 Hz, 1.0H), 1.34 (s, |
| | | | 3.2H), 1.17 (s, 3.0H), 1.08 ppm (s, 3.0H) |
| 74 | Similar to | m/z 456.2 | ¹H NMR (400 MHz, CHLOROFORM-d) δ = 7.65 |
| | Example 6 | [M + H]⁺ | (d, J = 1.2 Hz, 1H), 7.40-7.34 (m, 2H), 7.33-7.28 |
| | | | (m, 2H), 7.27-7.23 (m, 1H), 7.00 (s, 1H), 3.98 (d, |
| | | | J = 6.4 Hz, 1H), 3.81 (d, J = 6.4 Hz, 1H), 3.61 (d, J = |
| | | | 8.8 Hz, 1H), 3.56 (t, J = 2.8 Hz, 1H), 3.52 (br d, J = |
| | | | 8.0 Hz, 1H), 3.50-3.46 (m, 2H), 3.40 (td, J = 4.4, |
| | | | 8.7 Hz, 1H), 2.65 (s, 3H), 2.43 (s, 4H), 2.40-2.33 |
| | | | (m, 1H), 2.31-2.23 (m, 1H), 2.19 (d, J = 8.0 Hz, |
| | | | 1H), 1.47 (s, 3H) |
| 75 | Similar to | m/z 457.2 | ¹H NMR (400 MHz, CHLOROFORM-d) δ = 7.63 |
| | Example 6 | [M + H]⁺ | (s, 1H), 7.41-7.28 (m, 4H), 7.27-7.21 (m, 1H), |
| | | | 6.93 (s, 1H), 6.07 (br s, 1H), 4.10 (s, 2H), 3.58 (d, |
| | | | J = 8.8 Hz, 1H), 3.54-3.43 (m, 2H), 3.38 (dt, J = 3.6, |
| | | | 8.8 Hz, 1H), 2.63 (s, 3H), 2.60-2.55 (m, 2H), 2.49 |
| | | | (br dd, J = 1.2, 4.4 Hz, 2H), 2.41-2.32 (m, 1H), |
| | | | 2.29-2.20 (m, 1H), 2.03 (s, 3H), 1.46 (s, 3H) |
| 76 | Similar to | m/z 443.1 | ¹H NMR (400 MHz, CHLOROFORM-d) δ = 7.66 (s, |
| | Example 6 | [M + H]⁺ | 1H), 7.41-7.35 (m, 2H), 7.34-7.27 (m, 3H), 7.05 |
| | | | (s, 1H), 3.99 (d, J = 6.6 Hz, 1H), 3.72-3.66 (m, 2H), |
| | | | 3.62 (d, J = 8.9 Hz, 1H), 3.59-3.47 (m, 3H), 3.42 |
| | | | (dt, J = 3.6, 8.7 Hz, 1H), 2.69 (s, 3H), 2.52 (dd, J = 3.0, |
| | | | 8.0 Hz, 1H), 2.40 (td, J = 8.2, 12.1 Hz, 1H), 2.33-2.24 |
| | | | (m, 2H), 1.48 (s, 3H) |
| 77 | Similar to | m/z 457.2 | ¹H NMR (400 MHz, CHLOROFORM-d) δ = 7.67- |
| | Example 6 | [M + H]⁺ | 7.60 (m, 1H), 7.40-7.34 (m, 2H), 7.34-7.29 (m, |
| | | | 2H), 7.27-7.23 (m, 1H), 7.00-6.90 (m, 1H), 4.77 |
| | | | (br d, J = 6.4 Hz, 1H), 4.34 (br s, 1H), 4.27 (s, 1H), |
| | | | 4.18-4.06 (m, 1H), 3.92 (br s, 1H), 3.79 (s, 3H), 3.64- |
| | | | 3.54 (m, 2H), 3.52-3.45 (m, 2H), 3.44-3.34 (m, |
| | | | 1H), 2.62 (s, 3H), 2.52 (br d, J = 6.0 Hz, 1H), 2.44- |
| | | | 2.31 (m, 1H), 2.30-2.20 (m, 1H), 2.16-2.07 (m, |
| | | | 1H), 1.49-1.45 (m, 3H) |
| 436 | Similar to | m/z 431.2 | ¹H NMR (400 MHz, CHLOROFORM-d) δ = 13.29- |
| | Example 6 | [M + H]+ | 12.28 (m, 1H), 7.92 (d, J = 1.6 Hz, 1H), 7.37-7.29 |
| | | | (m, 2H), 7.27 (s, 3H), 7.08 (s, 1H), 4.08-4.00 (m, |
| | | | 1H), 3.99-3.92 (m, 2H), 3.79 (br d, J = 6.4 Hz, 1H), |
| | | | 3.55-3.49 (m, 1H), 3.45 (d, J = 2.8 Hz, 1H), 3.31- |
| | | | 3.20 (m, 1H), 2.58 (s, 3H), 2.43-2.35 (m, 4H), 2.14 |
| | | | (d, J = 8.0 Hz, 1H), 1.41 (d, J = 7.2 Hz, 3H) |
| 437 | Similar to | m/z 393.1 | ¹H NMR (400 MHz, CHLOROFORM-d) δ = 8.31 (br |
| | Example 6 | [M + H]+ | s, 1H), 7.93 (br s, 1H), 7.38-7.29 (m, 2H), 7.27 (br |
| | | | s, 3H), 7.07 (br s, 1H), 5.74 (br s, 1H), 4.12 (br d, J = |
| | | | 6.0 Hz, 1H), 4.07-4.01 (m, 1H), 4.00-3.91 (m, 2H), |
| | | | 3.36 (br s, 1H), 3.31-3.19 (m, 1H), 3.03 (br s, 1H), |
| | | | 2.54 (s, 3H), 2.14 (br d, J = 5.6 Hz, 1H), 1.99 (br d, |
| | | | J = 8.0 Hz, 1H), 1.41 (br d, J = 6.8 Hz, 3H) |

### Example 7

### Tert-butyl N-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]carbamate (Compound 7)

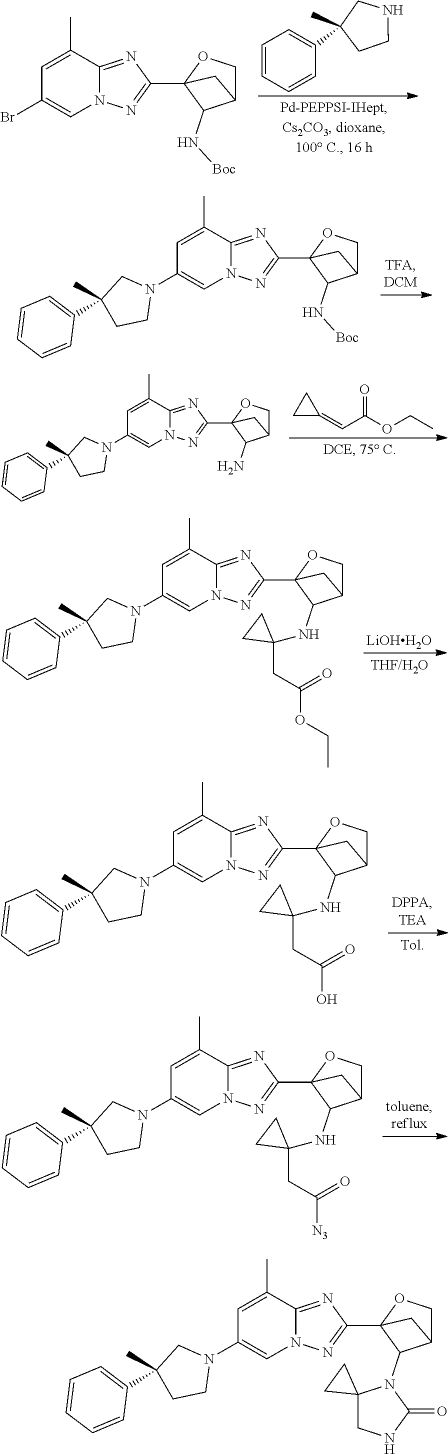

### Step 1: tert-butyl (1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamate

To a solution of (3R)-3-methyl-3-phenyl-pyrrolidine (354.57 mg, 2.20 mmol, 1 eq) and tert-butyl N-[1-(6-bromo-8-methyl-[1,2,4]triazolo[1,5-a]pyridin-2-yl)-2-oxabicyclo[2.1.1]hexan-5-yl]carbamate (900 mg, 2.20 mmol, 1 eq) in dioxane (12 mL) was added 1,3-bis[2,6-bis(1-propylbutyl)phenyl]-4,5-dichloro-2H-imidazol-1-ium-2-ide;3-chloropyridine;dichloropalladium (106.96 mg, 109.95 mol, 0.05 eq) and Cs₂CO₃ (2.15 g, 6.60 mmol, 3 eq). The mixture was stirred at 100° C. for 12 h under N₂. LCMS showed starting material was consumed and one main peak with desired mass was detected. The reaction mixture was concentrated under the reduced pressure to give the product. The residue was purified by prep-HPLC (FA condition column: CD05-Phenomenex luna C18 150*40*10 um; mobile phase: [water(FA)-ACN]; gradient:53%-83% B over 11 min). to give compound tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (810 mg, 75% yield) was obtained as a yellow solid.

¹H NMR (400 MHz, CHLOROFORM-d) δ=7.65 (s, 1H), 7.41-7.34 (m, 2H), 7.33-7.29 (m, 2H), 7.27-7.22 (m, 1H), 6.96 (br s, 1H), 4.13 (br d, J=5.2 Hz, 1H), 3.98 (br d, J=6.4 Hz, 1H), 3.88 (br d, J=6.0 Hz, 1H), 3.59 (br d, J=8.8 Hz, 1H), 3.55-3.44 (m, 2H), 3.38 (dt, J=3.6, 8.4 Hz, 1H), 3.10 (br s, 1H), 2.64 (s, 3H), 2.42-2.32 (m, 1H), 2.26 (dt, J=3.6, 7.6 Hz, 1H), 2.15 (br d, J=6.4 Hz, 1H), 1.84 (d, J=8.4 Hz, 1H), 1.46 (s, 3H), 1.41 (s, 9H).

### Step 2: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine

To a solution of tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (810 mg, 1.65 mmol, 1 eq) in DCM (3 mL) was added TFA (2.30 g, 20.19 mmol, 1.5 mL, 12.21 eq). The mixture was stirred at 25° C. for 0.5 h. LCMS showed starting material was consumed and one main peak with desired mass was detected. The reaction mixture was concentrated under reduced pressure to give a residue. The pH was adjusted to around 7 by progressively aq. NaHCO₃, and then extracted with EtOAc (10 mL×3). The combined organic layers were washed with brine (5 mL), dried over Na₂SO₄, filtered and concentrated under reduced pressure to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (612 mg, 95% yield) was obtained as a yellow solid.

LC-MS [ESI, M+1]: 390.2.

### Step 3: ethyl 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetate

To a solution of 1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-amine (100 mg, 256.74 mol, 1 eq) and ethyl 2-cyclopropylideneacetate (32.39 mg, 256.74 mol, 1 eq) in DCE (2 mL). The mixture was stirred at 75° C. for 1 h. LCMS showed starting material was consumed and one main peak with desired mass was detected. The reaction was added water (5 mL) and then extracted with EtOAc (10 mL×3). The combined organic phase was washed with brine (5 mL), dried with anhydrous Na₂SO₄, filtered and concentrated in vacuum. The residue was purified by prep-TLC (SiO2, Petroleum ether/Ethyl acetate=0:1, Rf=0.23) to give compound ethyl 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetate (85 mg, 64% yield) was obtained as a yellow gum.

LC-MS [ESI, M+1]: 516.3.

### Step 4: 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetic acid

To a solution of ethyl 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetate (85 mg, 164.84 mol, 1 eq) in THF (1 mL) was added LiOH·H₂O (27.67 mg, 659.37 mol, 4 eq) in H₂O (0.3 mL). The mixture was stirred at 25° C. for 1 h. LCMS showed starting material was consumed and one main peak with desired mass was detected. The reaction mixture was poured into 2 mL water and acidified with 2M HCl to pH around 4 and then the reaction mixture was concentrated under the reduced pressure to give compound 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetic acid (76 mg, crude) was obtained as a yellow gum.

LC-MS [ESI, M+1]: 488.2.

### Step 5: 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetyl azide

To a solution of 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetic acid (20 mg, 41.02 mol, 1 eq) in Tol. (1 mL) was added DPPA (12.42 mg, 45.12 mol, 9.74 L, 1.1 eq) and TEA (4.57 mg, 45.12 mol, 6.28 μL, 1.1 eq). The mixture was stirred at 25° C. for 1 h. LCMS showed starting material was consumed and one main peak with desired mass was detected. The reaction mixture was concentrated under the reduced pressure to give compound 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetyl azide (25 mg, crude) was obtained as a yellow gum.

LC-MS [ESI, M-11]: 502.3.

### Step 6: 4-[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]-4,6-diazaspiro[2.4]heptan-5-one

To a solution of 2-[1-[[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropyl]acetyl azide (25 mg, 48.77 mol, 1 eq) in Tol. (1 mL). The mixture was stirred at 100° C. for 1 h. LCMS showed starting material was consumed and one main peak with desired mass was detected. The reaction mixture was concentrated under the reduced pressure to give the product. The residue was purified by prep-HPLC (FA condition column: Phenomenex luna C18 150*25 mm*10 um; mobile phase: [water(FA)-ACN]; gradient:35%-65% B over 10 min) to give compound 4-[1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexan-5-yl]-4,6-diazaspiro[2.4]heptan-5-one (3.09 mg, 11% yield FA) was obtained as a yellow solid.

LC-MS [ESI, M+1]: 485.2.

¹H NMR (400 MHz, CHLOROFORM-d) δ=8.11 (s, 1H), 7.65 (s, 1H), 7.42-7.34 (m, 2H), 7.34-7.29 (m, 2H), 7.26-7.22 (m, 1H), 7.12 (br s, 1H), 6.95 (br s, 1H), 4.61 (br s, 1H), 4.31 (br d, J=6.0 Hz, 1H), 3.99 (br d, J=6.0 Hz, 1H), 3.59 (br s, 2H), 3.55-3.35 (m, 4H), 3.11 (br d, J=8.0 Hz, 1H), 2.63 (s, 3H), 2.42-2.33 (m, 1H), 2.28-2.20 (m, 1H), 2.11 (br d, J=7.2 Hz, 1H), 1.84 (br d, J=8.4 Hz, 1H), 1.46 (s, 3H), 1.15-1.05 (m, 1H), 0.60-0.46 (m, 1H), 0.20-0.03 (m, 2H).

| Cmpd. No. | Synthesis | LC/MS | ¹H NMR |
|---|---|---|---|
| 78 | Similar to | m/z 485.3 | ¹H NMR (400 MHz, CHLOROFORM-d) δ = |
| | Example 7 | [M + H]⁺ | 8.07 (s, 1H), 7.64 (d, J = 1.2 Hz, 1H), 7.40-7.34 |
| | | | (m, 2H), 7.33-7.29 (m, 2H), 7.27-7.23 (m, 1H), |
| | | | 6.95 (s, 1H), 4.30 (br s, 1H), 4.11-4.05 (m, 1H), |
| | | | 4.05-3.97 (m, 2H), 3.63-3.56 (m, 2H), 3.55- |
| | | | 3.49 (m, 2H), 3.49-3.45 (m, 1H), 3.42-3.34 (m, |
| | | | 2H), 2.62 (s, 3H), 2.37 (td, J = 8.4, 12.0 Hz, 1H), |
| | | | 2.28-2.22 (m, 1H), 2.11 (br dd, J = 3.2, 8.0 Hz, |
| | | | 1H), 1.93 (br d, J = 8.4 Hz, 1H), 1.46 (s, 3H), |
| | | | 0.79-0.69 (m, 2H), 0.68-0.62 (m, 1H), 0.57- |
| | | | 0.48 (m, 1H) |
| 79 | Similar to | m/z 501.3 | ¹H NMR (400 MHz, METHANOL-d4) δ = 7.78 |
| | Example 7 | [M + H]⁺ | (d, J = 1.2 Hz, 1H), 7.42-7.29 (m, 4H), 7.27- |
| | | | 7.17 (m, 2H), 4.13 (d, J = 6.4 Hz, 1H), 4.07-3.95 |
| | | | (m, 2H), 3.62 (t, J = 3.2 Hz, 1H), 3.60-3.53 (m, |
| | | | 3H), 3.42 (dt, J = 3.6, 8.8 Hz, 1H), 2.60 (s, 3H), |
| | | | 2.42-2.26 (m, 2H), 2.20 (dd, J = 2.8, 8.0 Hz, |
| | | | 1H), 1.84 (d, J = 8.0 Hz, 1H), 1.45 (s, 3H), 1.25 |
| | | | (s, 3H), 1.11 (s, 3H) |

### Example 8

### 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methylamino]pyridine-2-carboxylic acid (Compound 8)

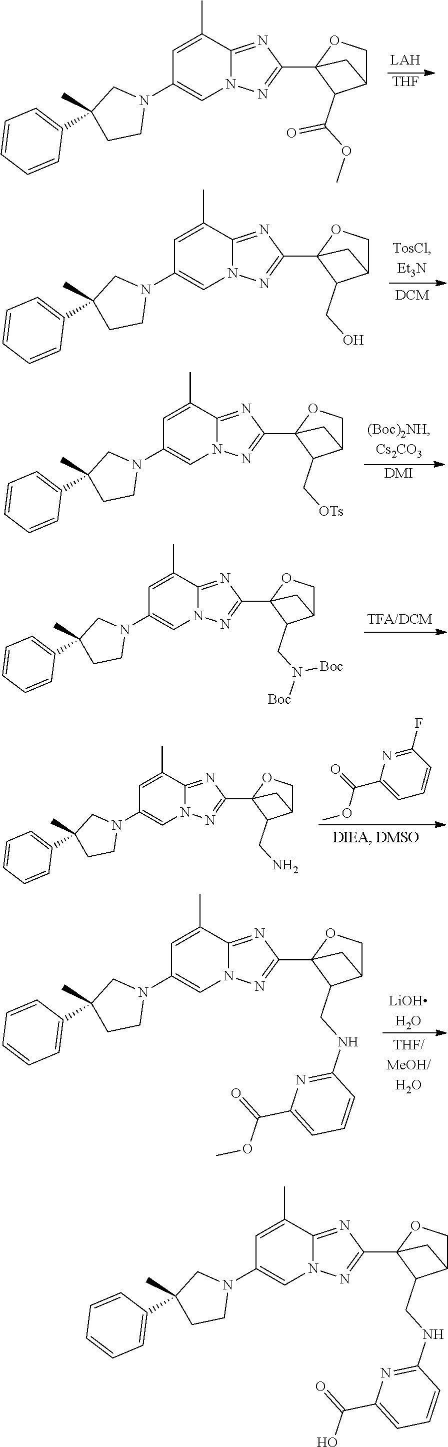

### Step 1: [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanol

To a solution of LiAlH₄ (2.5 M, 924.83 μL, 2 eq) in THF (6 mL) was added methyl 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carboxylate (500 mg, 1.16 mmol, 1 eq) in five portions at 0° C. under N₂. The reaction mixture was stirred at 25° C. for 1 h under N₂. LCMS showed starting material was consumed and one main peak with desired mass was detected. After the reaction mixture was cooled to 0° C., the reaction mixture was quenched by addition of (1 mL) of H₂O, followed by 1 mL of 15% aqueous NaOH and 3 mL of H₂O. and then extracted with EtOAc (10 mL×3). The combined organic phase was washed with brine (5 mL), dried with anhydrous Na₂SO₄, filtered and concentrated under reduced pressure to give compound [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanol (400 mg, crude) was obtained as a yellow solid.

LC-MS [ESI, M+1]: 405.2.

### Step 2: [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methyl 4-methylbenzenesulfonate

To a solution of [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanol (200 mg, 494.43 mol, 1 eq), DMAP (6.04 mg, 49.44 mol, 0.1 eq) and TEA (150.10 mg, 1.48 mmol, 206.46 L, 3 eq) in DCM (3 mL) was added TosCl (188.53 mg, 988.87 mol, 2 eq) at 0° C. The mixture was stirred at 25° C. for 1 h. LCMS showed starting material was consumed and one main peak with desired mass was detected. The reaction mixture was concentrated under the reduced pressure to give the product. The reaction was added water (5 mL) and then extracted with ethyl acetate(5 mL×3). The combined organic phase was washed with brine (5 mL), dried with anhydrous Na₂SO₄, filtered and concentrated in vacuum. The residue was purified by prep-TLC (SiO₂, PE:EtOAc=0:1, Rf=0.5) to give compound [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methyl 4-methylbenzenesulfonate (200 mg, 72% yield) was obtained as a green gum

¹H MR (400 MHz, CHLOROFORM-d) δ=7.77 (d, J=8.4 Hz, 2H), 7.60 (d, J=1.6 Hz, 1H), 7.40-7.34 (m, 2H), 7.33-7.29 (m, 4H), 7.27-7.23 (m, 1H), 6.96 (s, 1H), 4.09-4.02 (m, 1H), 3.94 (d, J=6.8 Hz, 1H), 3.77 (d, J=6.8 Hz, 1H), 3.59 (d, J=8.8 Hz, 1H), 3.55-3.44 (m, 2H), 3.38 (dt, J=3.6, 8.8 Hz, 1H), 3.06 (t, J=3.2 Hz, 1H), 2.85 (br d, J=2.8 Hz, 1H), 2.63 (s, 3H), 2.43 (s, 3H), 2.41-2.32 (m, 1H), 2.32-2.21 (m, 2H), 1.95 (d, J=7.6 Hz, 1H), 1.60-1.54 (m, 1H), 1.46 (s, 3H).

### Step 3: tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl]carbamate

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl 4-methylbenzenesulfonate (100 mg, 178.99 mol, 1 eq) in DMI (1 mL) was added Cs₂CO₃ (174.96 mg, 536.97 mol, 3 eq) and tert-butyl N-tert-butoxycarbonylcarbamate (77.78 mg, 357.98 mol, 2 eq). The mixture was stirred at 75° C. for 1 h. LCMS showed starting material was consumed and one main peak with desired mass was detected. The residue was purified by prep-TLC (SiO₂, PE:EtOAc=0:1, Rf=0.7) to give compound tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methyl]carbamate (93 mg, 86% yield) was obtained as a yellow gum.

LC-MS [ESI, M+1]: 604.3.

### Step 4: [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanamine

To a solution of tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methyl]carbamate (93 mg, 154.04 mol, 1 eq) in DCM (1 mL) was added TFA (767.50 mg, 6.73 mmol, 0.5 mL, 43.70 eq). The mixture was stirred at 25° C. for 1 h. LCMS showed starting material was consumed and one main peak with desired mass was detected. The reaction mixture was concentrated under the reduced pressure to give the product. The residue was purified by prep-HPLC (NH₄HCO3 condition column: Waters Xbridge BEH C18 150*25 mm*5 um; mobile phase: [water(NH₄HCO3)-ACN]; gradient:35%-65% B over 10 min) to give compound [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanamine (40 mg, 50% yield, TFA) was obtained as a yellow gum.

LC-MS [ESI, M+1]: 404.2.

### Step 5: methyl 6-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methylamino]pyridine-2-carboxylate

To a solution of [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methanamine (30 mg, 57.97 mol, 1 eq, TFA) and methyl 6-fluoropyridine-2-carboxylate (17.98 mg, 115.93 mol, 2 eq) in DMSO (1 mL) was added K₂CO₃ (24.03 mg, 173.90 mol, 3 eq). The mixture was stirred at 100° C. for 1 h. LCMS showed starting material was consumed and one main peak with desired mass was detected. The reaction was added water (5 mL) and then extracted with ethyl acetate(10 mL×3). The combined organic phase was washed with brine (5 mL), dried with anhydrous Na₂SO₄, filtered and concentrated under reduced pressure to give compound methyl 6-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methylamino]pyridine-2-carboxylate (35 mg, crude) was obtained as a yellow oil.

LC-MS [ESI, M+1]: 539.2.

### Step 6: 6-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methylamino]pyridine-2-carboxylic acid

To a solution of methyl 6-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methylamino]pyridine-2-carboxylate (35 mg, 64.98 mol, 1 eq) in THF (1 mL) was added LiOH·H2O (8.18 mg, 194.94 mol, 3 eq) in H₂O (0.3 mL). The mixture was stirred at 25° C. for 1 h. LCMS showed starting material was consumed and one main peak with desired mass was detected. The reaction mixture was poured into 3 mL water and acidified with 2M HCl to pH around 4 and then the reaction mixture was concentrated under the reduced pressure to give the product. The residue was purified by prep-HPLC (FA condition column: Phenomenex luna C18 150*25 mm*10 um; mobile phase: [water(FA)-ACN]; gradient:27%-57% B over 8 min) to give compound 6-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methylamino]pyridine-2-carboxylic acid (3.77 mg, 11% yield) was obtained as a yellow solid.

LC-MS [ESI, M+1]: 525.2.

¹H NMR (400 MHz, CHLOROFORM-d) δ=7.62 (br s, 1H), 7.53 (br d, J=1.2 Hz, 1H), 7.42-7.29 (m, 5H), 7.24 (br s, 1H), 6.95 (br s, 1H), 6.73-6.59 (m, 1H), 4.17-3.91 (m, 2H), 3.59 (br d, J=8.8 Hz, 1H), 3.55-3.32 (m, 5H), 3.21-3.07 (m, 1H), 2.78-2.69 (m, 1H), 2.64 (br s, 3H), 2.41-2.34 (m, 1H), 2.28-2.23 (m, 1H), 2.14 (br d, J=2.4 Hz, 1H), 2.06 (br d, J=3.6 Hz, 1H), 1.47 (s, 3H).

| Cmpd. No. | Synthesis | LC/MS | ¹H NMR |
|---|---|---|---|
| 80 | Similar to | m/z 404.2 | ¹H NMR (400 MHz, DMSO-d6) δ = 8.27 (br s, 3H), |
| | Example 8 | [M + H]⁺ | 7.99 (s, 1H), 7.46-7.29 (m, 5H), 7.27-7.20 (m, 1H), |
| | | | 3.92-3.81 (m, 2H), 3.62-3.55 (m, 1H), 3.54-3.44 |
| | | | (m, 2H), 3.39-3.31 (m, 1H), 3.08 (br s, 1H), 2.93 (td, |
| | | | J = 6.4, 12.4 Hz, 1H), 2.81 (td, J = 6.4, 12.8 Hz, 1H), |
| | | | 2.63-2.52 (m, 4H), 2.25 (br t, J = 6.8 Hz, 2H), 2.14- |
| | | | 2.06 (m, 1H), 1.85 (d, J = 7.6 Hz, 1H), 1.34 (s, 3H) |
| 81 | Similar to | m/z 460.3 | ¹H NMR (400 MHz, CHLOROFORM-d) δ = 7.70 (br |
| | Example 8 | [M + H]⁺ | d, J = 4.8 Hz, 1H), 7.54 (s, 1H), 7.36-7.20 (m, 5H), |
| | | | 6.91 (s, 1H), 3.89 (br d, J = 3.2 Hz, 2H), 3.64 (ddd, J = |
| | | | 4.0, 9.2, 12.8 Hz, 1H), 3.52 (br d, J = 8.8 Hz, 1H), 3.48- |
| | | | 3.37 (m, 2H), 3.32 (dt, J = 3.6, 8.8 Hz, 1H), 3.00 (br |
| | | | t, J = 12.0 Hz, 1H), 2.89 (br s, 1H), 2.55 (s, 3H), 2.38- |
| | | | 2.27 (m, 2H), 2.22-2.11 (m, 3H), 2.01-1.94 (m, 2H), |
| | | | 1.39 (s, 3H), 1.06 (br t, J = 7.6 Hz, 3H) |

### Example 9

### 8-methyl-6-[(3R)-3-methyl-3-phenyl-pyrrolidin-1-yl]-2-[5-[(5-methyl-1H-1,2,4-triazol-3-yl)methyl]-2-oxabicyclo[2.1.1]hexan-1-yl]-[1,2,4]triazolo[1,5-a]pyridine (Compound 9)

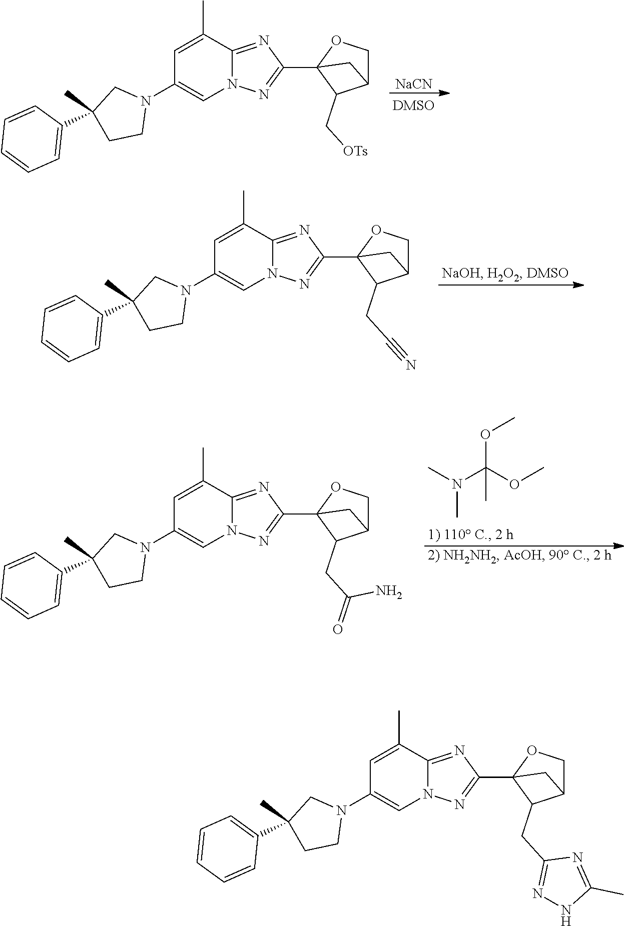

### Step 1: 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetonitrile

To a solution of [1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]methyl 4-methylbenzenesulfonate (400 mg, 715.96 mol, 1 eq) in DMSO (3 mL) was added KCN (186.48 mg, 2.86 mmol, 122.68 μL, 4 eq) slowly. The mixture was stirred at 100° C. for 3 h. LCMS showed starting material was consumed and one main peak with desired mass was detected. The reaction was added water (10 mL) and then extracted with EtOAc (10 mL×3). The combined organic phase was washed with brine (10 mL), dried over anhydrous Na₂SO₄, filtered and concentrated under reduced pressure to give compound 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetonitrile (266 mg, 83% yield) was obtained as a yellow gum. LC-MS [ESI, M+1]: 414.2.

### Step 2: 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetamide

To a solution of 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetonitrile (266 mg, 643.27 mol, 1 eq) and K₂CO₃ (88.90 mg, 643.27 mol, 1 eq) in DCM (3 mL) was added dropwise H₂O₂ (2.19 g, 19.30 mmol, 1.85 mL, 30% purity, 30 eq) at 0° C. The mixture was stirred at 25° C. for 0.5 h LCMS showed starting material was consumed and one main peak with desired mass was detected. The reaction was quenched by Sodium Nitrite aq. (10 mL) slowly and then extracted with DCM (10 mL×3). The combined organic phases were dried over Na₂SO₄, filtered and concentrated in vacuum. The residue was purified by prep-HPLC (NH₄HCO3 condition column: Waters xbridge 150*25 mm 10 um; mobile phase: [water(NH₄HCO3)-ACN]; gradient:24%-54% B over 10 min) to give compound 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetamide (168.09 mg, 61% yield) was obtained as a white solid.

LC-MS [ESI, M+1]: 432.1.

¹H NMR (400 MHz, CHLOROFORM-d) δ=7.61 (s, 1H), 7.57 (br s, 1H), 7.41-7.32 (m, 2H), 7.32-7.28 (m, 2H), 7.27-7.22 (m, 1H), 6.95 (s, 1H), 5.26 (br s, 1H), 3.98 (d, J=6.4 Hz, 1H), 3.85 (d, J=6.4 Hz, 1H), 3.59 (d, J=8.8 Hz, 1H), 3.55-3.44 (m, 2H), 3.38 (dt, J=3.6, 8.8 Hz, 1H), 2.98 (t, J=2.8 Hz, 1H), 2.79-2.64 (m, 2H), 2.58 (s, 3H), 2.37 (td, J=8.4, 12.0 Hz, 1H), 2.30-2.09 (m, 3H), 2.00 (d, J=7.6 Hz, 1H), 1.46 (s, 3H).

### Step 3: 8-methyl-6-[(3R)-3-methyl-3-phenyl-pyrrolidin-1-yl]-2-[5-[(5-methyl-1H-1,2,4-triazol-3-yl)methyl]-2-oxabicyclo[2.1.1]hexan-1-yl]-[1,2,4]triazolo[1,5-a]pyridine

To a solution of 2-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]acetamide (40 mg, 92.69 mol, 1 eq) in 1,1-dimethoxy-N,N-dimethyl-ethanamine (911.00 mg, 6.84 mmol, 1 mL, 73.79 eq), the mixture was stirred at 110° C. for 2 h. Then the mixture was concentrated under reduced pressure to give a residue. To a solution of this residue in EtOH (1 mL) was added HOAc (209.80 mg, 3.49 mmol, 0.2 mL, 37.69 eq) and N₂H₄·H₂O (206.40 mg, 3.50 mmol, 0.2 mL, 85% purity, 37.81 eq) at −10° C. And then the mixture was stirred at 90° C. for 2 h. LCMS showed starting material was consumed and one main peak with desired mass was detected. The reaction was quenched by ice-water (5 mL) slowly and then extracted with EtOAc (5 mL×3). The combined organic phase was washed with brine (5 mL), dried with anhydrous Na₂SO₄, filtered and concentrated in vacuum. The residue was purified by prep-HPLC (FA condition column: Phenomenex luna C18 150*25 mm*10 um; mobile phase: [water(FA)-ACN]; gradient:24%-54% B over 10 min) to give compound 8-methyl-6-[(3R)-3-methyl-3-phenyl-pyrrolidin-1-yl]-2-[5-[(5-methyl-1H-1,2,4-triazol-3-yl)methyl]-2-oxabicyclo[2.1.1]hexan-1-yl]-[1,2,4]triazolo[1,5-a]pyridine (9.12 mg, 21% yield) was obtained as a white

LC-MS [ESI, M+1]: 470.2.

¹H NMR (400 MHz, CHLOROFORM-d) δ=7.65 (s, 1H), 7.41-7.34 (m, 2H), 7.34-7.29 (m, 2H), 7.25 (br s, 1H), 7.02 (s, 1H), 4.05 (d, J=6.4 Hz, 1H), 3.94 (d, J=6.4 Hz, 1H), 3.60 (d, J=8.8 Hz, 1H), 3.56-3.45 (m, 2H), 3.40 (dt, J=3.6, 8.8 Hz, 1H), 3.13 (dd, J=9.2, 16.4 Hz, 1H), 2.97 (t, J=2.8 Hz, 1H), 2.82 (br d, J=16.0 Hz, 1H), 2.75 (s, 3H), 2.66 (br d, J=7.2 Hz, 1H), 2.43-2.34 (m, 4H), 2.26 (ddd, J=3.6, 7.6, 11.6 Hz, 1H), 2.10-2.03 (m, 2H), 1.47 (s, 3H).

| Cmpd. No. | Synthesis | LC/MS | ¹H NMR |
|---|---|---|---|
| 82 | Similar to | m/z 470.2 | ¹H NMR (400 MHz, CHLOROFORM-d) δ = 8.48 (br |
| | Example 9 | [M + H]⁺ | s, 1H), 7.63 (s, 1H), 7.41-7.34 (m, 2H), 7.33-7.29 |
| | | | (m, 2H), 7.26-7.22 (m, 1H), 6.97 (s, 1H), 4.41 (br dd, |
| | | | J = 7.2, 14.4 Hz, 1H), 4.17-4.07 (m, 2H), 4.01 (br d, |
| | | | J = 6.8 Hz, 1H), 3.60 (br d, J = 8.8 Hz, 1H), 3.54-3.45 |
| | | | (m, 2H), 3.39 (dt, J = 3.6, 8.8 Hz, 1H), 3.08 (br s, 1H), |
| | | | 2.90 (br s, 1H), 2.60 (s, 3H), 2.43-2.33 (m, 4H), 2.30- |
| | | | 2.20 (m, 2H), 2.03 (d, J = 8.0 Hz, 1H), 1.46 (s, 3H) |
| 83 | Similar to | m/z 470.1 | ¹H NMR (400 MHz, CHLOROFORM-d) δ = 9.24 (br |
| | Example 9 | [M + H]⁺ | s, 5H), 8.07 (d, J = 7.6 Hz, 1H), 7.55 (br s, 1H), 7.41- |
| | | | 7.34 (m, 2H), 7.33-7.29 (m, 2H), 7.28-7.24 (m, 1H), |
| | | | 7.10 (s, 1H), 4.29 (dd, J = 8.4, 14.4 Hz, 1H), 4.18-4.00 |
| | | | (m, 3H), 3.65-3.58 (m, 1H), 3.57-3.47 (m, 2H), 3.41 |
| | | | (dq, J = 3.6, 8.4 Hz, 1H), 3.16 (br s, 1H), 3.11 (br dd, |
| | | | J = 5.2, 7.6 Hz, 1H), 2.64 (d, J = 6.8 Hz, 3H), 2.59 (s, |
| | | | 3H), 2.40 (td, J = 8.4, 12.4 Hz, 1H), 2.34-2.23 (m, |
| | | | 2H), 2.08 (br d, J = 8.0 Hz, 1H), 1.46 (s, 3H) |
| 84 | Similar to | m/z 487.2 | ¹H NMR (400 MHz, CHLOROFORM-d) δ = 8.21- |
| | Example 9 | [M + H]⁺ | 8.03 (m, 1H), 7.64 (s, 1H), 7.41-7.33 (m, 2H), 7.33- |
| | | | 7.29 (m, 2H), 7.26-7.21 (m, 1H), 6.94 (s, 1H), 6.33 |
| | | | (br s, 1H), 4.04-3.97 (m, 1H), 3.97-3.91 (m, 1H), |
| | | | 3.59 (d, J = 8.8 Hz, 1H), 3.55-3.44 (m, 2H), 3.38 (dt, |
| | | | J = 3.6, 8.8 Hz, 1H), 3.31 (br t, J = 4.4 Hz, 2H), 3.15 |
| | | | (d, J = 3.2 Hz, 2H), 2.99 (br s, 1H), 2.66 (br s, 2H), 2.62 |
| | | | (s, 4H), 2.57-2.50 (m, 2H), 2.41-2.34 (m, 1H), 2.29- |
| | | | 2.20 (m, 2H), 1.94 (d, J = 7.6 Hz, 1H), 1.46 (s, 3H) |
| 85 | Similar to | m/z 432.1 | ¹H NMR (400 MHz, CHLOROFORM-d) δ = 7.61 (s, |
| | Example 9 | [M + H]⁺ | 1H), 7.57 (br s, 1H), 7.41-7.32 (m, 2H), 7.32-7.28 |
| | | | (m, 2H), 7.27-7.22 (m, 1H), 6.95 (s, 1H), 5.26 (br s, |
| | | | 1H), 3.98 (d, J = 6.4 Hz, 1H), 3.85 (d, J = 6.4 Hz, 1H), |
| | | | 3.59 (d, J = 8.8 Hz, 1H), 3.55-3.44 (m, 2H), 3.38 (dt, |
| | | | J = 3.6, 8.8 Hz, 1H), 2.98 (t, J = 2.8 Hz, 1H), 2.79- |
| | | | 2.64 (m, 2H), 2.58 (s, 3H), 2.37 (td, J = 8.4, 12.0 Hz, |
| | | | 1H), 2.30-2.09 (m, 3H), 2.00 (d, J = 7.6 Hz, 1H), 1.46 |
| | | | (s, 3H) |
| 86 | Similar to | m/z 470.2 | ¹H NMR (400 MHz, CHLOROFORM-d) δ = 7.90 (s, |
| | Example 9 | [M + H]⁺ | 1H), 7.74 (s, 1H), 7.65 (s, 1H), 7.59 (s, 1H), 7.40-7.34 |
| | | | (m, 2H), 7.33-7.29 (m, 2H), 7.27-7.21 (m, 1H), 6.92 |
| | | | (s, 1H), 4.14 (d, J = 6.4 Hz, 1H), 4.07-4.03 (m, 1H), |
| | | | 4.03-3.98 (m, 1H), 3.84 (s, 1H), 3.76 (s, 2H), 3.58 (br |
| | | | d, J = 8.4 Hz, 1H), 3.55-3.48 (m, 1H), 3.48-3.44 (m, |
| | | | 1H), 3.38 (dt, J = 3.6, 8.4 Hz, 1H), 3.06 (br s, 1H), 2.94 |
| | | | (br s, 1H), 2.92-2.79 (m, 3H), 2.61 (d, J = 10.4 Hz, |
| | | | 3H), 2.41-2.32 (m, 1H), 2.29-2.16 (m, 2H), 1.98 (br |
| | | | dd, J = 7.6, 15.1 Hz, 1H), 1.46 (s, 3H) |
| 87 | Similar to | m/z 486.1 | ¹H NMR (400 MHz, CHLOROFORM-d) δ = 7.60 |
| | Example 9 | [M + H]⁺ | (br s, 1H), 7.40-7.33 (m, 2H), 7.33-7.29 (m, 2H), |
| | | | 7.26-7.23 (m, 1H), 6.93 (br s, 1H), 4.73 (br d, J = |
| | | | 12.8 Hz, 1H), 4.15-3.97 (m, 2H), 3.87-3.73 (m, |
| | | | 1H), 3.58 (br d, J = 8.4 Hz, 1H), 3.54-3.43 (m, 2H), |
| | | | 3.38 (br s, 2H), 3.13 (br d, J = 7.6 Hz, 1H), 3.04- |
| | | | 2.94 (m, 1H), 2.57 (br s, 3H), 2.35 (br s, 4H), 2.29- |
| | | | 2.22 (m, 2H), 1.46 (s, 3H) |
| 88 | Similar to | m/z 472.2 | ¹H NMR (400 MHz, CHLOROFORM-d) δ = 9.19 (br |
| | Example 9 | [M + H]⁺ | s, 1H), 7.60 (s, 1H), 7.46 (s, 1H), 7.40-7.34 (m, 2H), |
| | | | 7.34-7.29 (m, 2H), 7.27-7.23 (m, 1H), 6.94 (s, 1H), |
| | | | 4.08-3.98 (m, 2H), 3.82-3.70 (m, 2H), 3.58 (d, J = |
| | | | 8.9 Hz, 1H), 3.54-3.43 (m, 2H), 3.39 (dt, J = 3.6, 8.6 |
| | | | Hz, 1H), 3.08-3.01 (m, 1H), 2.77-2.70 (m, 1H), |
| | | | 2.61 (s, 3H), 2.37 (td, J = 8.2, 12.0 Hz, 1H), 2.29- |
| | | | 2.17 (m, 2H), 1.98 (d, J = 7.8 Hz, 1H), 1.46 (s, 3H) |
| 89 | Similar to | m/z 486.2 | ¹H NMR (400 MHz, CHLOROFORM-d) δ = 7.67 (s, |
| | Example 9 | [M + H]⁺ | 1H), 7.43-7.33 (m, 2H), 7.32-7.29 (m, 1H), 7.26 |
| | | | (br d, J = 7.6 Hz, 2H), 6.96 (s, 1H), 3.86 (s, 2H), 3.66- |
| | | | 3.59 (m, 1H), 3.54 (br d, J = 8.8 Hz, 1H), 3.50- |
| | | | 3.41 (m, 1H), 3.38-3.28 (m, 1H), 2.67 (s, 3H), 2.45- |
| | | | 2.23 (m, 6H), 1.89 (br dd, J = 7.6, 14.5 Hz, 2H), 0.83- |
| | | | 0.65 (m, 3H) |
| 90 | Similar to | m/z 499.1 | ¹H NMR (400 MHz, CHLOROFORM-d) δ = 8.13 (s, |
| | Example 9 | [M + H]⁺ | 1H), 7.57 (s, 1H), 7.40-7.34 (m, 2H), 7.34-7.29 (m, |
| | | | 2H), 7.27-7.23 (m, 1H), 6.98-6.84 (m, 2H), 5.69 |
| | | | (br s, 1H), 4.48 (dd, J = 7.2, 14.4 Hz, 1H), 4.24 (dd, |
| | | | J = 6.0, 14.4 Hz, 1H), 4.10-4.05 (m, 1H), 4.05-4.01 |
| | | | (m, 1H), 3.58 (d, J = 8.8 Hz, 1H), 3.54-3.44 (m, 2H), |
| | | | 3.38 (dt, J = 3.2, 8.8 Hz, 1H), 3.09-3.02 (m, 1H), |
| | | | 2.97-2.90 (m, 1H), 2.60 (s, 3H), 2.44-2.33 (m, 1H), |
| | | | 2.29-2.19 (m, 2H), 2.02 (br d, J = 7.6 Hz, 1H), 1.46 |
| | | | (s, 3H) |
| 91 | Similar to | m/z 500.1 | ¹H NMR (400 MHz, CHLOROFORM-d) δ = 8.32 (s, |
| | Example 9 | [M + H]+ | 1H), 7.64 (s, 1H), 7.38-7.31 (m, 2H), 7.31-7.28 (m, |
| | | | 2H), 7.26-7.21 (m, 1H), 6.95 (s, 1H), 4.53-4.38 (m, |
| | | | 2H), 4.14 (br d, J = 6.8 Hz, 1H), 4.06 (br d, J = 6.8 |
| | | | Hz, 1H), 3.57 (br d, J = 8.8 Hz, 1H), 3.52-3.43 (m, |
| | | | 2H), 3.39-3.33 (m, 1H), 3.00 (br s, 1H), 2.84 (br s, |
| | | | 1H), 2.61 (s, 3H), 2.37-2.31 (m, 1H), 2.27-2.21 (m, |
| | | | 1H), 2.20-2.15 (m, 1H), 1.96 (br d, J = 7.8 Hz, 1H), |
| | | | 1.43 (s, 3H) |
| 92 | Similar to | m/z 499.1 | ¹H NMR (400 MHz, CHLOROFORM-d) δ = 8.29 (s, |
| | Example 9 | [M + H]+ | 1H), 7.62 (s, 1H), 7.41-7.34 (m, 2H), 7.34-7.29 (m, |
| | | | 2H), 7.24 (br s, 1H), 7.01 (br s, 2H), 5.63 (br s, 1H), |
| | | | 4.64-4.45 (m, 2H), 4.17-4.05 (m, 2H), 3.60 (br d, J = |
| | | | 8.4 Hz, 1H), 3.56-3.45 (m, 2H), 3.44-3.35 (m, |
| | | | 1H), 3.06 (br s, 1H), 2.92 (br s, 1H), 2.68-2.59 (m, |
| | | | 3H), 2.43-2.32 (m, 1H), 2.30-2.20 (m, 2H), 2.04 |
| | | | (br d, J = 7.8 Hz, 1H), 1.47 (s, 3H) |

### Example 10

### N-[5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazol-3-yl]acetamide (Compound 10)

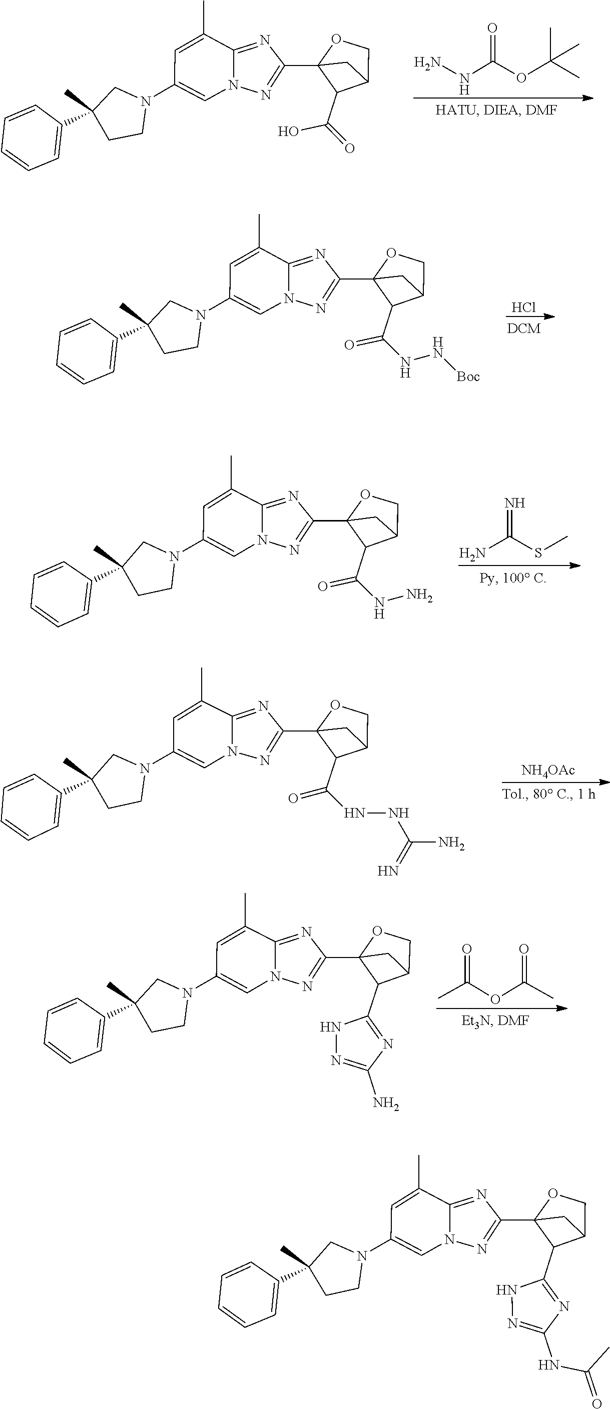

### Step 1: tert-butyl N-[[1-[8-methyl-6-[rac-(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]carbamate

To a solution of 1-[8-methyl-6-[rac-(3R)-3-methyl-3-phenyl-pyrrolidin-1-yl]-[1,2,4]triazolo[1,5-a]pyridine-2-yl]-2-oxabicyclo[2.1.1]hexane-5-carboxylic acid (800 mg, 1.91 mmol, 1 eq) and tert-butyl N-aminocarbamate (303.17 mg, 2.29 mmol, 1.2 eq), HATU (1.09 g, 2.87 mmol, 1.5 eq) in DMF (5 mL) was added DIEA (247.07 mg, 1.91 mmol, 332.97 L, 1 eq) The mixture was stirred at 25° C. for 1 h. LCMS showed starting material was consumed and one main peak with desired mass was detected. The reaction was added water (10 mL) and then extracted with EtOAc (10 mL×3). The combined organic phase was washed with brine (15 mL), dried with anhydrous Na₂SO₄, filtered and concentrated in vacuum. The residue was purified by column chromatography (SiO₂, PE:EtOA=0:1, Rf=0.6) to give compound tert-butyl N-[[1-[8-methyl-6-[rac-(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]carbamate (1 g, 98% yield) was obtained as a yellow gum.

¹H NMR (400 MHz, CHLOROFORM-d) δ=8.02 (s, 1H), 7.64 (s, 1H), 7.41-7.34 (m, 2H), 7.33-7.29 (m, 2H), 7.26-7.22 (m, 1H), 7.02 (s, 1H), 6.66-6.43 (m, 1H), 4.23 (br s, 1H), 4.00 (d, J=6.0 Hz, 1H), 3.60 (d, J=8.8 Hz, 1H), 3.57-3.45 (m, 2H), 3.44-3.36 (m, 2H), 3.01 (br s, 2H), 2.62 (s, 3H), 2.38 (td, J=8.4, 12.0 Hz, 1H), 2.30-2.18 (m, 2H), 2.08-2.03 (m, 3H), 1.47 (s, 9H).

### Step 2: 1-[8-methyl-6-[rac-(3R)-3-methyl-3-phenyl-pyrrolidin-1-yl]-[1,2,4]triazolo[1,5-a]pyridin-2-yl]-2-oxabicyclo[2.1.1]hexane-5-carbohydrazide

To a solution of tert-butyl N-[[1-[8-methyl-6-[rac-(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]carbamate (1 g, 1.88 mmol, 1 eq) in DCM (10 mL) was added HCl/dioxane (4 M, 5 mL, 10.65 eq). The mixture was stirred at 25° C. for 1 h. LCMS showed starting material was consumed and one main peak with desired mass was detected. The reaction mixture was concentrated under the reduced pressure to give the product. The pH was adjusted to around 7 by progressively aq. NaHCO₃, and then extracted with EtOAc (10 mL×3). The combined organic layers were washed with brine (5 mL), dried over Na₂SO₄, filtered and concentrated under reduced pressure to give compound 1-[8-methyl-6-[rac-(3R)-3-methyl-3-phenyl-pyrrolidin-1-yl]-[1,2,4]triazolo[1,5-a]pyridine-2-yl]-2-oxabicyclo[2.1.1]hexane-5-carbohydrazide (620 mg, crude) was obtained as a yellow solid.

LC-MS [ESI, M+1]: 433.2.

### Step 3: 1-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]guanidine

To a solution of 1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbohydrazide (620 mg, 1.43 mmol, 1 eq) and 2-methylisothiourea (193.83 mg, 2.15 mmol, 1.5 eq) in Py (10 mL). The mixture was stirred at 100° C. for 12 h. LCMS showed starting material was consumed and one main peak with desired mass was detected. The reaction mixture was concentrated under the reduced pressure to give the product. The crude product was triturated with PE at 25° C. for 30 min to give compound 1-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]guanidine (800 mg, crude) was obtained as a green solid.

LC-MS [ESI, M+1]: 475.2.

### Step 4: 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazol-3-amine (Compound 438)

To a solution of 1-[[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexane-5-carbonyl]amino]guanidine (800 mg, 1.69 mmol, 1 eq) in dioxane (10 mL) was added NH₄Oac (519.78 mg, 6.74 mmol, 4 eq). The mixture was stirred at 100° C. for 1 h. LCMS showed starting material was consumed and one main peak with desired mass was detected. The reaction mixture was concentrated under the reduced pressure to give the product. The residue was purified by prep-HPLC (HCl condition column: CD05-Phenomenex luna C18 150*40*10 um; mobile phase: [water(HCl)-ACN]; gradient:25%-55% B over 10 min) to give compound 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazol-3-amine (170 mg, 21% yield, HCl) was obtained as a yellow solid.

LC-MS [ESI, M+1]: 457.2.

¹H NMR (400 MHz, CHLOROFORM-d) δ=8.10-7.99 (m, 1H), 7.57 (br s, 1H), 7.34 (br d, J=7.2 Hz, 3H), 7.30 (br d, J=6.4 Hz, 2H), 7.25 (br s, 1H), 4.26 (br d, J=0.8 Hz, 1H), 4.15-3.90 (m, 3H), 3.70-3.42 (m, 6H), 2.82 (br s, 3H), 2.49-2.22 (m, 3H), 2.15 (br d, J=3.2 Hz, 1H), 1.44 (br s, 3H).

### Step 5: N-[5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]acetamide

To a solution of 5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazol-3-amine (25 mg, 50.71 mol, 1 eq, HCl) and TEA (16.62 mg, 164.25 mol, 22.86 μL, 3.24 eq) in DMF (1 mL) was added acetyl acetate (6.15 mg, 60.24 mol, 5.66 μL, 1.19 eq). The mixture was stirred at 25° C. for 1 h. LCMS showed starting material was consumed and one main peak with desired mass was detected. The reaction mixture was filtered. The residue was purified by prep-HPLC (FA condition column: CD01-Phenomenex luna C18 150*25*10 um; mobile phase: [water(FA)-ACN]; gradient:30%-60% B over 10 min) to give compound N-[5-[1-[8-methyl-6-[(3R)-3-methyl-3-phenyl-pyrrolidin-1-yl]-[1,2,4]triazolo[1,5-a]pyridine-2-yl]-2-oxabicyclo[2.1.1]hexan-5-yl]-1H-1,2,4-triazol-3-yl]acetamide (1.87 mg, 7% yield) was obtained as a white solid.

LC-MS [ESI, M+1]: 499.2.

¹H NMR (400 MHz, CHLOROFORM-d) δ=7.84-7.60 (m, 1H), 7.42-7.29 (m, 4H), 7.27-7.14 (m, 2H), 4.01 (br d, J=5.2 Hz, 2H), 3.66 (br s, 1H), 3.55 (br s, 3H), 3.40 (br s, 2H), 2.59 (br s, 3H), 2.47 (br d, J=3.2 Hz, 1H), 2.39-2.25 (m, 2H), 2.13 (br s, 3H), 2.04-1.88 (m, 1H), 1.44 (s, 3H).

| Cmpd. No. | Synthesis | LC/MS | ¹H NMR |
|---|---|---|---|
| 93 | Similar to | m/z 549.2 | ¹H NMR (400 MHz, CHLOROFORM-d) δ = 7.70 |
| | Example 10 | [M + H]⁺ | (br s, 1H), 7.41-7.29 (m, 4H), 7.26-7.20 (m, |
| | | | 1H), 6.92 (s, 1H), 6.26 (br d, J = 8.8 Hz, 2H), 4.36 |
| | | | (br d, J = 3.6 Hz, 1H), 3.99 (br d, J = 6.0 Hz, 1H), |
| | | | 3.60-3.45 (m, 4H), 3.44-3.32 (m, 4H), 2.62 (s, |
| | | | 3H), 2.42-2.30 (m, 2H), 2.28-2.18 (m, 1H), |
| | | | 1.96 (br d, J = 7.6 Hz, 1H), 1.46 (s, 3H), 1.32 (dt, |
| | | | J = 1.2, 7.2 Hz, 3H) |

### Example 11

### 8-methyl-6-[(3R)-3-methyl-3-phenyl-pyrrolidin-1-yl]-2-[5-(1-methyltriazol-4-yl)-2-oxabicyclo[2.1.1]hexan-1-yl]-[1,2,4]triazolo[1,5-a]pyridine (Compound 11)

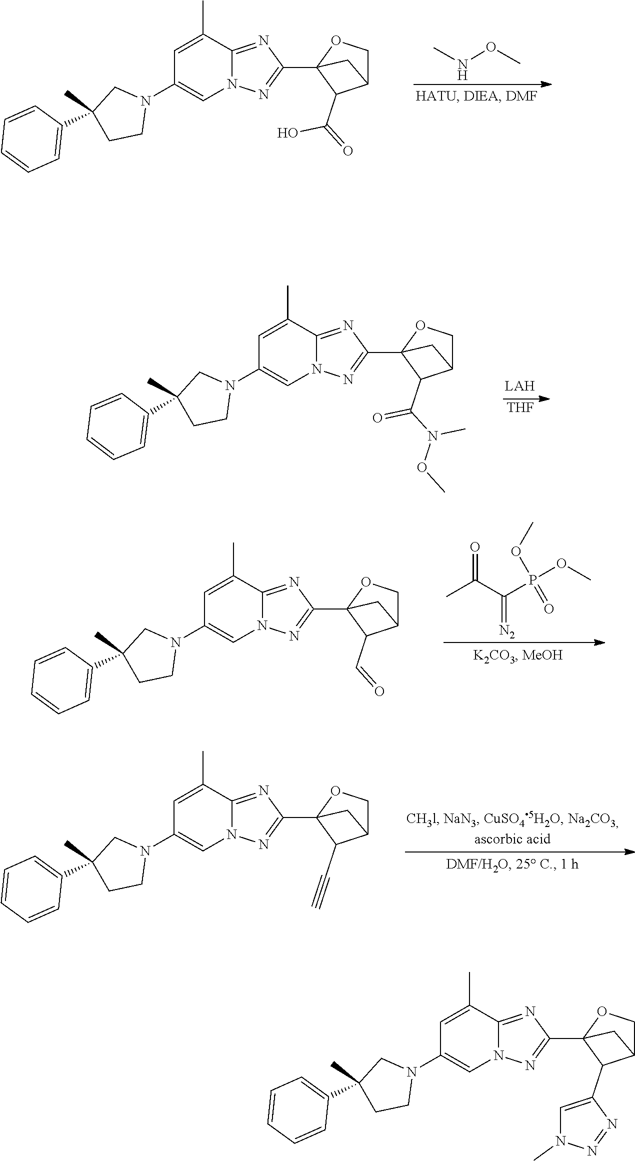

### Step 1: N-methoxy-N-methyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (550 mg, 1.31 mmol, 1 eq) and N-methoxymethanamine (120.42 mg, 1.97 mmol, 1.5 eq) in DMF (5 mL) was added HATU (999.44 mg, 2.63 mmol, 2 eq) and then was added DIEA (509.56 mg, 3.94 mmol, 686.74 μL, 3 eq) after 0.1 h. The mixture was stirred at 25° C. for 1 h. LCMS showed starting material was consumed completely and the desired mass was detected. The reaction mixture was added water, the aqueous phase was extracted with EtOAc (10 mL×3). The combined organic phase was washed with brine (30 mL), dried with anhydrous Na₂SO₄, filtered and concentrated in vacuum. The residue was purified by flash silica gel chromatography (ISCO®; 20 g SepaFlash® Silica Flash Column, Eluent of 0~100% EtOAc:PE @80 mL/min) to give compound N-methoxy-N-methyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (400 mg, 65% yield) as a green gum.

LC-MS [ESI, M+1]: 462.2.

¹H NMR (400 MHz, CHLOROFORM-d) δ=7.64 (s, 1H), 7.40-7.34 (m, 2H), 7.33-7.29 (m, 2H), 7.26-7.22 (m, 1H), 6.93 (s, 1H), 4.29 (s, 1H), 4.13 (q, J=7.2 Hz, 1H), 4.00 (d, J=6.0 Hz, 1H), 3.97-3.97 (m, 1H), 3.57 (d, J=8.4 Hz, 1H), 3.53-3.45 (m, 2H), 3.43-3.30 (m, 5H), 3.08 (s, 3H), 2.60 (s, 3H), 2.41-2.31 (m, 2H), 2.29-2.21 (m, 1H), 1.88 (d, J=7.2 Hz, 1H), 1.46 (s, 3H).

### Step 2: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbaldehyde

To a solution of N-methoxy-N-methyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (200 mg, 433.32 mol, 1 eq) in THF (4 mL) was added LAH (32.89 mg, 866.64 mol, 2 eq). The mixture was stirred at 0° C. for 0.3 h. LCMS showed starting material was consumed completely and the desired mass was detected. After the reaction mixture was cooled to 0° C., the reaction mixture was quenched by addition of (0.2 mL) of H₂O, followed by 0.2 mL of 15% aqueous NaOH and 0.6 mL of H₂O. After being stirred at room temperature for 0.5 h, the solid was removed by filtration. The filtrate was concentrated to dryness to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbaldehyde (160 mg, crude) as a yellow oil.

LC-MS [ESI, M+1]: 403.2.

### Step 3: 2-(5-ethynyl-2-oxabicyclo[2.1.1]hexan-1-yl)-8-methyl-6-[(3R)-3-methyl-3-phenyl-pyrrolidin-1-yl]-[1,2,4]triazolo[1,5-a]pyridine

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbaldehyde (150 mg, 372.68 mol, 1 eq) and 1-diazo-1-dimethoxyphosphoryl-propan-2-one (143.19 mg, 745.36 mol, 2 eq) in MeOH (4 mL) was added K₂CO₃ (103.02 mg, 745.36 mol, 2 eq). The mixture was stirred at 25° C. for 2 h. LCMS showed starting material was consumed completely and the desired compound was detected. The reaction was concentrated under reduced pressure to give a residue. The residue was purified by column chromatography (SiO₂, PE:EtOAc=1:2, Rf=0.42) and the eluted with EtOAc (100 mL) to give compound 2-(5-ethynyl-2-oxabicyclo[2.1.1]hexan-1-yl)-8-methyl-6-[(3R)-3-methyl-3-phenyl-pyrrolidin-1-yl]-[1,2,4]triazolo[1,5-a]pyridine (60 mg, 41% yield) as a yellow oil.

LC-MS [ESI, M+1]: 399.2.

¹H NMR (400 MHz, CHLOROFORM-d) δ=7.66 (s, 1H), 7.40-7.33 (m, 2H), 7.33-7.28 (m, 2H), 7.27-7.22 (m, 1H), 6.93 (s, 1H), 4.32 (d, J=6.0 Hz, 1H), 4.05 (d, J=6.0 Hz, 1H), 3.58 (d, J=8.8 Hz, 1H), 3.53-3.43 (m, 2H), 3.37 (dt, J=8.8, 4.0 Hz, 1H), 3.19-3.10 (m, 2H), 2.63 (s, 3H), 2.40-2.32 (m, 1H), 2.31-2.19 (m, 3H), 1.91 (d, J=7.2 Hz, 1H), 1.46 (s, 3H).

### Step 4: 8-methyl-6-[(3R)-3-methyl-3-phenyl-pyrrolidin-1-yl]-2-[5-(1-methyltriazol-4-yl)-2-oxabicyclo[2.1.1]hexan-1-yl]-[1,2,4]triazolo[1,5-a]pyridine

To a solution of 2-(5-ethynyl-2-oxabicyclo[2.1.1]hexan-1-yl)-8-methyl-6-[(3R)-3-methyl-3-phenyl-pyrrolidin-1-yl]-[1,2,4]triazolo[1,5-a]pyridine (15 mg, 37.64 mol, 1 eq) in DMF (0.8 mL) and H₂O (0.2 mL) was added CuSO₄·5H₂O (4.70 mg, 18.82 mol, 0.5 eq) sodium; (2R)-2-[(2R)-3,4-dihydroxy-5-oxo-2H-furan-2-yl]-2-hydroxy-ethanolate (5.97 mg, 30.11 mol, 0.8 eq), NaN₃ (12.24 mg, 188.21 mol, 5 eq), Mel (6.41 mg, 45.17 mol, 2.81 μL, 1.2 eq) and Na₂CO₃ (11.97 mg, 112.92 μmol, 3 eq). The mixture was stirred at 25° C. for 1 h. LCMS showed starting material was consumed completely and the desired compound was detected. The reaction was quenched H₂O (2 mL) and then extracted with EtOAc (5 mL×3). The combined organic phase was washed with brine (5 mL×3), dried over anhydrous Na₂SO₄, filtered and concentrated in vacuum. Then the reaction aqueous phase was quenched by NaClO (1 mL). The residue was purified by prep-HPLC (column: CD02-Waters Xbidge BEH C18 150*25*10 um; mobile phase: [water(NH₄HCO3)-ACN]; gradient:32%-62% B over 10 min) to give compound 8-methyl-6-[(3R)-3-methyl-3-phenyl-pyrrolidin-1-yl]-2-[5-(1-methyltriazol-4-yl)-2-oxabicyclo[2.1.1]hexan-1-yl]-[1,2,4]triazolo[1,5-a]pyridine (9.81 mg, 56% yield) as a white solid.

LC-MS [ESI, M+1]: 456.2.

¹H NMR (400 MHz, CHLOROFORM-d) δ=7.80 (s, 1H), 7.63 (s, 1H), 7.39-7.33 (m, 2H), 7.33-7.28 (m, 2H), 7.27-7.22 (m, 1H), 6.93 (s, 1H), 4.06-3.96 (m, 4H), 3.82 (d, J=2.8 Hz, 1H), 3.77 (d, J=6.4 Hz, 1H), 3.58 (d, J=8.8 Hz, 1H), 3.52-3.43 (m, 2H), 3.41-3.31 (m, 2H), 2.63 (s, 3H), 2.47 (dd, J=7.2, 2.8 Hz, 1H), 2.36 (td, J=12.4, 8.4 Hz, 1H), 2.29-2.19 (m, 1H), 2.05 (d, J=7.2 Hz, 1H), 1.45 (s, 3H).

### Example 12

### [1-(hydroxymethyl)-3-[8-methyl-6-[(3R)-3-methyl-3-phenyl-pyrrolidin-1-yl]-[1,2,4]triazolo[1,5-a]pyridin2-yl]-2-oxabicyclo[2.1.1]hexane-4-carboxamide (Compound 12)

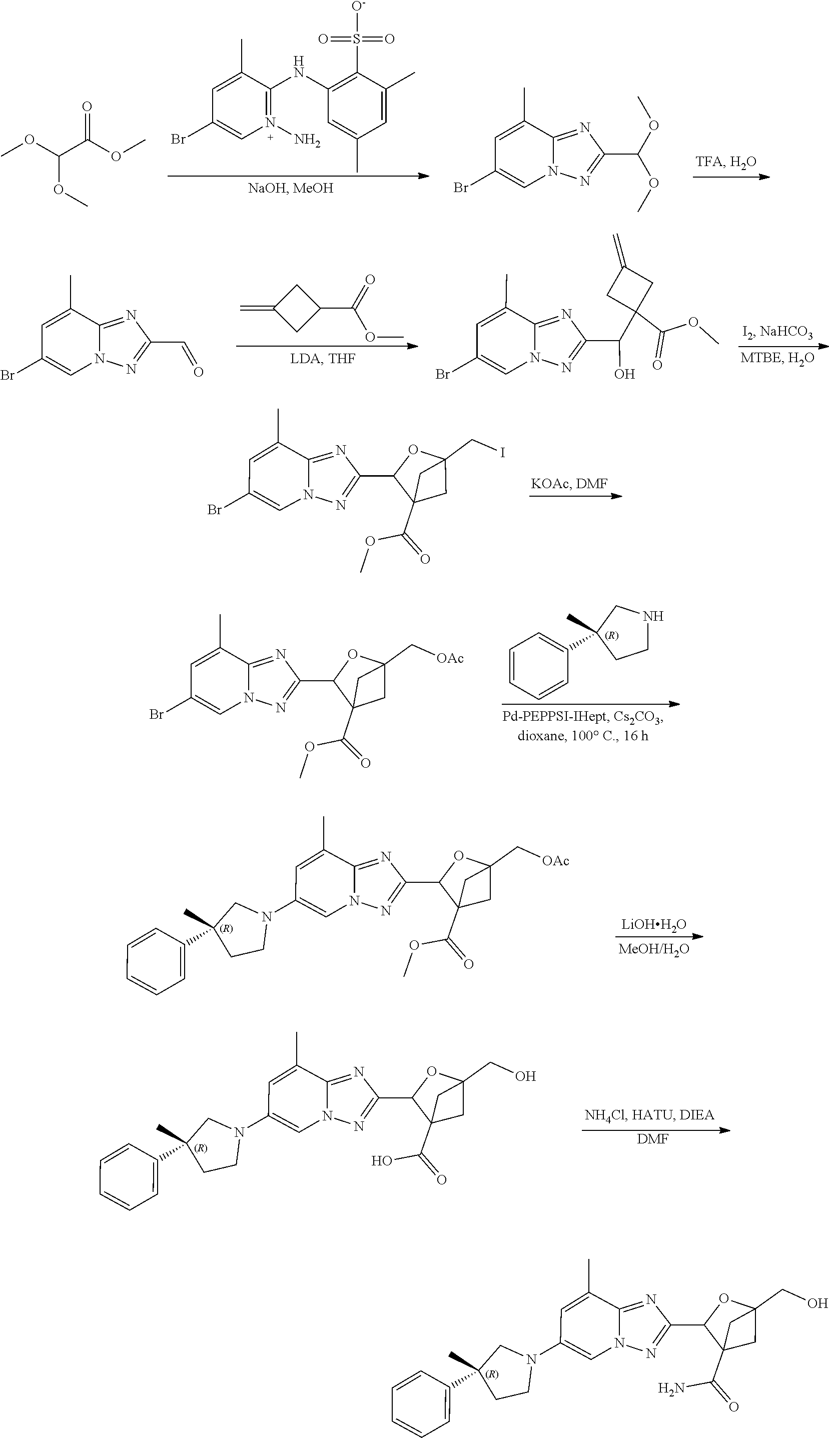

### Step 1: 6-bromo-2-(dimethoxymethyl)-8-methyl-[1,2,4]triazolo[1,5-a]pyridine

To a solution of 5-bromo-3-methyl-pyri din-1l-ium-1,2-diamine;2,4,6-trimethylbenzenesulfonate (10 g, 24.86 mmol, 1 eq) and methyl 2,2-dimethoxyacetate (6.67 g, 49.71 mmol, 6.10 mL, 2 eq) in MeOH (200 mL) was added NaH (1.49 g, 37.29 mmol, 1.5 eq. The mixture was stirred at 70° C. for 12 hr. LCMS showed starting material consumed competely. Several new peaks were shown on LCMS and 5500 of desired mass was detected. The reaction mixture was filtered and concentrated under reduced pressure to give a residue. The residue was purified by flash silica gel chromatography (ISCO®; 40 g SepaFlash® Silica Flash Column, Eluent of 0-42% Ethyl acetate/Petroleum ethergradient @70 mL/min, PE:EtOAc=1:1, Rf=0.4) to give compound 6-bromo-2-(dimethoxymethyl)-8-methyl-[1,2,4]triazolo[1,5-a]pyridine (5.36 g, 75% yield) as a yellow solid.

LC-MS [ESI, M-32]: 253.6.

¹H NMR (400 MHz, CHLOROFORM-d) δ=8.57 (d, J=0.4 Hz, 1H), 7.40 (d, J=0.8 Hz, 1H), 5.73 (d, J=2.0 Hz, 1H), 3.49 (d, J=2.0 Hz, 6H), 2.65 (s, 3H).

### Step 2: 6-bromo-8-methyl-[1,2,4]triazolo[1,5-a]pyridine-2-carbaldehyde

To a solution of 6-bromo-2-(dimethoxymethyl)-8-methyl-[1,2,4]triazolo[1,5-a]pyridine (6.2 g, 21.67 mmol, 1 eq) in H₂O (5 mL) was added dropwise TFA (20 mL) dropwise at 0° C. After addition, the mixture was stirred at 80° C. for 1 hr. LCMS showed starting material consumed competely. Several new peaks were shown on LCMS and 56% of desired mass was detected. The reaction mixture was concentrated under reduced pressure to give a residue. The residue was diluted with NaHCO₃50 mL and extracted with ethyl acetate 240 mL (80 mL×3). The combined organic layers were dried over anhydrous sodium sulfate, filtered and concentrated under reduced pressure to give a residue 6-bromo-8-methyl-[1,2,4]triazolo[1,5-a]pyridine-2-carbaldehyde (4.73 g, 62% yield) as a yellow solid.

LC-MS [ESI, M+1]: 239.9.

¹H NMR (400 MHz, CHLOROFORM-d) δ=10.22 (s, 1H), 8.67 (d, J=0.8 Hz, 1H), 7.51 (s, 1H), 2.71 (s, 3H).

### Step 3: methyl 1-[(6-bromo-8-methyl-[1,2,4]triazolo[1,5-a]pyridin-2-yl)-hydroxy-methyl]-3-methylenecyclobutanecarboxylate

To a solution of methyl 3-methylenecyclobutanecarboxylate (1.68 g, 13.33 mmol, 1 eq) in THF (200 mL) was added LDA (2 M, 16.66 mL, 2.5 eq) at −78° C. under N₂ atmosphere, after 1 hr, 6-bromo-8-methyl-[1,2,4]triazolo[1,5-a]pyridine-2-carbaldehyde (3.2 g, 13.33 mmol, 1 eq) was added in reaction mixture, the resulting mixture was stirred for another 1 hr at −78° C. LCMS showed 3% of starting material remained. Several new peaks were shown on LCMS and 29% of desired mass was detected. The reaction mixture was quenched by addition NH₄Cl 25 mL at −20° C., and then diluted with H₂O 100 mL and extracted with ethyl acetate 300 mL (100 mL×3). The combined organic layers were washed with brine 240 mL (80 mL×3), dried over anhydrous sodium sulfate, filtered and concentrated under reduced pressure to give a residue. The residue was purified by flash silica gel chromatography (ISCO®; 40 g SepaFlash® Silica Flash Column, Eluent of 0~50% Ethyl acetate/Petroleum ethergradient @60 mL/min, PE:EtOAc=1:1, Rf=0.5) to give compound methyl 1-[(6-bromo-8-methyl-[1,2,4]triazolo[1,5-a]pyridin-2-yl)-hydroxy-methyl]-3-methylenecyclobutanecarboxylate (970 mg, 17% yield) as a yellow solid.

LC-MS [ESI, M-32]: 366.8.

¹H NMR (400 MHz, CHLOROFORM-d) δ=8.52 (d, J=0.8 Hz, 1H), 7.37 (s, 1H), 5.19 (d, J=9.2 Hz, 1H), 4.84 (t, J=2.0 Hz, 2H), 3.89 (d, J=9.2 Hz, 1H), 3.77 (s, 3H), 3.26-3.12 (m, 2H), 3.10-2.95 (m, 2H), 2.60 (s, 3H).

### Step 4: methyl 3-(6-bromo-8-methyl-[1,2,4]triazolo[1,5-a]pyridin-2-yl)-1-(iodomethyl)-2-oxabicyclo[2.1.1]hexane-4-carboxylate

A mixture of methyl 1-[(6-bromo-8-methyl-[1,2,4]triazolo[1,5-a]pyridin-2-yl)-hydroxy-methyl]-3-methylenecyclobutanecarboxylate (1.41 g, 3.37 mmol, 1 eq) in H₂O (4 mL) and MTBE (12 mL) was added NaHCO₃ (566.74 mg, 6.75 mmol, 262.50 μL, 2 eq) and 12 (1.71 g, 6.75 mmol, 1.36 mL, 2 eq), and then the mixture was stirred at 25° C. for 1 hr. LCMS showed 13% of starting material remained. Several new peaks were shown on LCMS and 54% of desired mass was detected. The reaction mixture was quenched by addition Na₂SO₃ 20 mL at 0° C., and then diluted with H₂O 30 mL and extracted with ethyl acetate 120 mL (40 mL×3). The combined organic layers were washed with brine 90 mL (30 mL×3), dried over anhydrous sodium sulfate, filtered and concentrated under reduced pressure to give a residue. The residue was purified by flash silica gel chromatography (ISCO®; 40 g SepaFlash® Silica Flash Column, Eluent of 0~40% Ethyl acetate/Petroleum ethergradient @80 mL/min, PE:EtOAc=1:1, Rf=0.6) to give compound methyl 3-(6-bromo-8-methyl-[1,2,4]triazolo[1,5-a]pyridin-2-yl)-1-(iodomethyl)-2-oxabicyclo[2.1.1]hexane-4-carboxylate (1.44 g, 82% yield) as a yellow oil.

LC-MS [ESI, M+1]: 491.9.

¹H NMR (400 MHz, CHLOROFORM-d) δ=8.55 (s, 1H), 7.37 (s, 1H), 5.51 (s, 1H), 3.71 (s, 3H), 3.61-3.56 (m, 1H), 3.54-3.49 (m, 1H), 2.72-2.65 (m, 1H), 2.61 (s, 3H), 2.36-2.27 (m, 2H), 2.25 (d, J=8.0 Hz, 1H).

### Step 5: methyl 1-(acetoxymethyl)-3-(6-bromo-8-methyl-[1,2,4]triazolo[1,5-a]pyridin-2-yl)-2-oxabicyclo[2.1.1]hexane-4-carboxylate

To a solution of methyl 3-(6-bromo-8-methyl-[1,2,4]triazolo[1,5-a]pyridin-2-yl)-1-(iodomethyl)-2-oxabicyclo[2.1.1]hexane-4-carboxylate (142 mg, 288.56 mol, 1 eq) in DMF (2 mL) was added KOAc (42.48 mg, 432.83 μmol, 1.5 eq). The mixture was stirred at 80° C. for 12 hr. LCMS showed starting material consumed competely and 26% of desired mass was detected. The reaction mixture was diluted with H₂O 15 mL and extracted with ethyl acetate 60 mL (20 mL×3). The combined organic layers were washed with brine 75 mL (15 mL×5), dried over anhydrous sodium sulfate, filtered and concentrated under reduced pressure to give a residue. The residue was purified by prep-TLC (SiO₂, PE:EtOAc=1:1) to give compound methyl 1-(acetoxymethyl)-3-(6-bromo-8-methyl-[1,2,4]triazolo[1,5-a]pyridin-2-yl)-2-oxabicyclo[2.1.1]hexane-4-carboxylate (50 mg, 41% yield) as a light yellow oil.

LC-MS [ESI, M+1]: 424.8.

### Step 6: methyl 1-(acetoxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate

To a solution of methyl 1-(acetoxymethyl)-3-(6-bromo-8-methyl-[1,2,4]triazolo[1,5-a]pyridin-2-yl)-2-oxabicyclo[2.1.1]hexane-4-carboxylate (50 mg, 117.86 mol, 1 eq), (3R)-3-methyl-3-phenyl-pyrrolidine (19.00 mg, 117.86 mol, 1 eq) and Cs₂CO₃ (96.00 mg, 294.64 mol, 2.5 eq) in dioxane (1 mL) was added 1,3-bis[2,6-bis(1-propylbutyl)phenyl]-4,5-dichloro-2H-imidazol-1-ium-2-ide;3-chloropyridine;dichloropalladium (5.73 mg, 5.89 mol, 0.05 eq) under N₂ atmosphere. The suspension was degassed and purged with N₂ for 3 times. The mixture was stirred at 100° C. for 12 hr. LCMS showed starting material consumed competely. Several new peaks were shown on LCMS and 61% of desired compound was detected. The reaction mixture was filtered and diluted with H₂O 15 mL and extracted with ethyl acetate 60 mL (20 mL×3). The combined organic layers were washed with brine 45 mL (15 mL×3), dried over anhydrous sodium sulfate, filtered and concentrated under reduced pressure to give a residue methyl 1-(acetoxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (31 mg, 40% yield) as a light-yellow oil.

LC-MS [ESI, M+1]: 505.2.

¹H NMR (400 MHz, CHLOROFORM-d) δ=7.64 (s, 1H), 7.40-7.34 (m, 2H), 7.33-7.29 (m, 2H), 7.26-7.24 (m, 1H), 6.95 (br s, 1H), 5.52 (s, 1H), 4.44 (s, 2H), 3.73-3.69 (m, 3H), 3.62-3.57 (m, 1H), 3.53-3.44 (m, 2H), 3.42-3.34 (m, 1H), 2.73 (dt, J=2.4, 8.2 Hz, 1H), 2.63 (s, 3H), 2.40-2.34 (m, 1H), 2.33-2.24 (m, 3H), 2.21 (d, J=7.6 Hz, 1H), 2.12 (s, 3H), 1.46 (s, 3H).

### Step 7: 1-(hydroxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid

To a solution of methyl 1-(acetoxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (31 mg, 61.44 mol, 1 eq) in MeOH (0.6 mL) and H₂O (0.2 mL) was added LiOH (2.94 mg, 122.88 mol, 2 eq). The mixture was stirred at 25° C. for 1 hr. LCMS showed starting material consumed. Several new peaks were shown on LCMS and 76% of desired compound was detected. The reaction mixture was filtered and concentrated to give a residue 1-(hydroxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (16 mg, 46% yield) as a white oil.

LC-MS [ESI, M+1]: 449.2.

### Step 8: 1-(hydroxymethyl)-3-[8-methyl-6-[(3R)-3-methyl-3-phenyl-pyrrolidin-1-yl]-[1,2,4]triazolo[1,5-a]pyridin2-yl]-2-oxabicyclo[2.1.1]hexane-4-carboxamide

To a solution of 1-(hydroxymethyl)-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (16 mg, 35.67 mol, 1 eq) in DMF (1 mL) was added HATU (27.13 mg, 71.35 mol, 2 eq), DIEA (9.22 mg, 71.35 mol, 12.43 μL, 2 eq) and NH₄C₁ (2.86 mg, 53.51 mol, 1.5 eq) The mixture was stirred at 25° C. for 12 hr. LCMS showed starting material consumed. Several new peaks were shown on LCMS and 75% of desired compound was detected. The reaction mixture was filtered to give a residue. The residue was purified by prep-HPLC (FA condition, column: Welch Xtimate C18 150×25 mm×5 um; mobile phase: [water(FA)-ACN]; gradient:21%-51% B over 10 min) to give compound 1-(hydroxymethyl)-3-[8-methyl-6-[(3R)-3-methyl-3-phenyl-pyrrolidin-1-yl]-[1,2,4]triazolo[1,5-a]pyridin2-yl]-2-oxabicyclo[2.1.1]hexane-4-carboxamide (9.54 mg, 59% yield) as a yellow solid.

LC-MS [ESI, M+1]: 448.2.

¹H NMR (400 MHz, CHLOROFORM-d) δ=7.77 (br s, 1H), 7.67 (br s, 1H), 7.40-7.34 (m, 2H), 7.33-7.27 (m, 3H), 7.01 (br s, 1H), 5.54 (br s, 1H), 5.45 (s, 1H), 4.05-3.90 (m, 2H), 3.60 (br d, J=8.4 Hz, 1H), 3.56-3.45 (m, 2H), 3.40 (br d, J=2.5 Hz, 1H), 2.67-2.53 (m, 4H), 2.38 (br d, J=7.2 Hz, 2H), 2.28-2.22 (m, 2H), 2.15 (br d, J=6.0 Hz, 1H), 1.46 (s, 3H)

| Cmpd. No. | Synthesis | LC/MS | ¹H NMR |
|---|---|---|---|
| 94 | Similar to | m/z 514.2 | ¹H NMR (400 MHz, CHLOROFORM-d) δ = 8.22 |
| | Example 12 | [M + H]⁺ | (s, 1H), 7.70 (s, 1H), 7.42-7.27 (m, 5H), 7.02 (s, |
| | | | 1H), 5.39 (s, 1H), 4.54 (d, J = 1.6 Hz, 2H), 3.61 (br |
| | | | d, J = 8.9 Hz, 1H), 3.56-3.46 (m, 2H), 3.40 (dt, J = |
| | | | 3.6, 8.8 Hz, 1H), 2.58 (s, 3H), 2.48 (br d, J = 6.4 |
| | | | Hz, 1H), 2.43-2.35 (m, 4H), 2.31-2.23 (m, 1H), |
| | | | 2.21-2.09 (m, 3H), 1.47 (s, 3H) |
| 95 | Similar to | m/z 513.2 | ¹H NMR (400 MHz, CHLOROFORM-d) δ = 8.33 |
| | Example 12 | [M + H]⁺ | (s, 1H), 7.86 (br s, 1H), 7.67 (s, 1H), 7.40-7.29 |
| | | | (m, 4H), 7.27-7.23 (m, 1H), 6.98 (s, 1H), 5.58 (br |
| | | | s, 1H), 5.40 (s, 1H), 4.60-4.53 (m, 2H), 3.60 (br d, |
| | | | J = 8.8 Hz, 1H), 3.55-3.45 (m, 2H), 3.40 (br t, J = |
| | | | 8.0 Hz, 1H), 2.63-2.55 (m, 4H), 2.46-2.33 (m, |
| | | | 5H), 2.31-2.22 (m, 1H), 2.18-2.07 (m, 2H), 1.46 |
| | | | (s, 3H) |

### Example 13

### 3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-4H-1,2,4-oxadiazol-5-one (Compound 13)

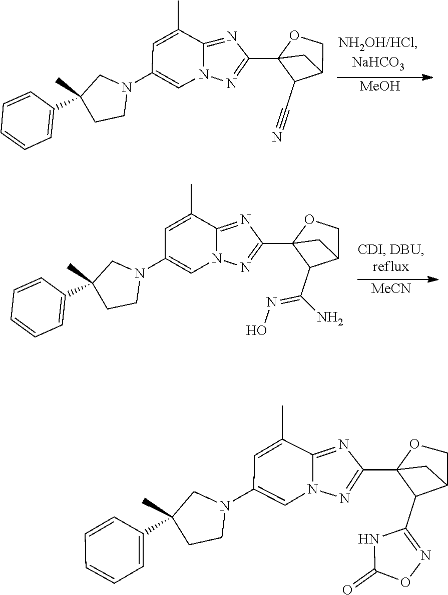 [SMILES: "MeOH" ; "MeCN" ; Cc1cc(N2CC[C@](C)(c3ccccc3)C2)cn2nc(C34CC(CO3)C4C#N)nc12 ; Cc1cc(N2CC[C@](C)(c3ccccc3)C2)cn2nc(C34CC(CO3)C4/C(N)=N/O)nc12 ; Cc1cc(N2CC[C@](C)(c3ccccc3)C2)cn2nc(C34CC(CO3)C4c3noc(=O)[nH]3)nc12]

### Step 1: N′-hydroxy-1-[8-methyl-6-[(3R)-3-methyl-3-phenyl-pyrrolidin-1-yl]-[1,2,4]triazolo[1,5-a]396yrrolid-2-yl]-2-oxabicyclo[2.1.1]hexane-5-carboxamidine

To a solution of 1-[8-methyl-6-[(3R)-3-methyl-3-phenyl-pyrrolidin-1-yl]-[1,2,4]triazolo[1,5-a]396yrrolid-2-yl]-2-oxabicyclo[2.1.1]hexane-5-carbonitrile (30 mg, 75.10 mol, 1 eq) in MeOH (1 mL) was added hydroxylamine; hydrochloride (10.44 mg, 150.19 mol, 2 eq) and NaHCO₃ (11.36 mg, 135.17 mol, 5.26 μL, 1.8 eq). The mixture was stirred at 70° C. for 12 h. LCMS showed starting material was consumed and the desired mass was detected. The reaction mixture was extracted with EtOAc (5 mL×3). The combined organic layers were washed with brine (5 mL), dried over anhydrous sodium sulfate, filtered and the filtrate was concentrated in vacuo to give compound N′-hydroxy-1-[8-methyl-6-[(3R)-3-methyl-3-phenyl-pyrrolidin-1-yl]-[1,2,4]triazolo[1,5-a]396 yrrolid-2-yl]-2-oxabicyclo[2.1.1]hexane-5-carboxamidine (30 mg, 92% yield) as a white solid.

LC-MS [ESI, M+1]: 433.2.

### Step 2: 3-[1-[8-methyl-6-[(3R)-3-methyl-3-phenyl-pyrrolidin-1-yl]-[1,2,4]triazolo[1,5-a]396 yrrolid-2-yl]-2-oxabicyclo[2.1.1]hexan-5-yl]-4H-1,2,4-oxadiazol-5-one

To a solution of N′-hydroxy-1-[8-methyl-6-[(3R)-3-methyl-3-phenyl-pyrrolidin-1-yl]-[1,2,4]triazolo[1,5-a]396 yrrolid-2-yl]-2-oxabicyclo[2.1.1]hexane-5-carboxamidine (30 mg, 69.36 mol, 1 eq) in ACN (1 mL) was added DBU (316.78 g, 2.08 mol, 3.14e-1 μL, 0.03 eq) and CDI (11.25 mg, 69.36 mol, 1 eq). The mixture was stirred at 80° C. for 12 h. LCMS showed starting material was consumed and the desired mass was detected. The mixture was concentrated in vacuo to give the residue. The residue was purified by prep-HPLC (column: Waters xbridge 150*25 mm 10 um; mobile phase: [water(NH₄HCO₃)—ACN]; gradient:25%-55% B over 11 min) to give compound 3-[1-[8-methyl-6-[(3R)-3-methyl-3-phenyl-pyrrolidin-1-yl]-[1,2,4]triazolo[1,5-a]397 yrrolid-2-yl]-2-oxabicyclo[2.1.1]hexan-5-yl]-4H-1,2,4-oxadiazol-5-one (1.67 mg, 5% yield) as a white solid.

LC-MS [ESI, M+1]: 459.2.

¹H NMR (400 MHz, CHLOROFORM-d) δ=7.64 (br s, 1H), 7.42-7.34 (m, 2H), 7.34-7.29 (m, 2H), 7.25 (br s, 1H), 7.01 (br s, 1H), 4.18 (br s, 1H), 4.03 (br d, J=6.0 Hz, 1H), 3.61 (br d, J=8.8 Hz, 1H), 3.57-3.45 (m, 3H), 3.41 (br d, J=7.4 Hz, 1H), 3.29 (br s, 1H), 2.62 (s, 3H), 2.44-2.35 (m, 2H), 2.32-2.18 (m, 2H), 1.47 (s, 3H).

| Cmpd. No. | Synthesis | LC/MS | ¹H NMR |
|---|---|---|---|
| 96 | Similar to | m/z 377.1 | ¹H NMR (400 MHz, DMSO-d₆) δ 8.40 (d, J = 2.0 Hz, |
| | Example 13 | [M + H]⁺ | 1H), 7.36-7.27 (m, 4H), 7.22 (td, J = 1.2, 2.4 Hz, 1H), |
| | | | 7.21-7.17 (m, 1H), 4.15 (s, 2H), 3.80-3.76 (m, 1H), |
| | | | 3.74-3.70 (m, 1H), 3.15 (d, J = 3.2 Hz, 1H), 2.68 (t, J = |
| | | | 3.2 Hz, 1H), 2.45 (s, 3H), 1.80-1.72 (m, 3H), 1.50 |
| | | | (d, J = 8.0 Hz, 1H), 1.06-1.02 (m, 2H), 1.00-0.96 |
| | | | (m, 2H). |
| 97 | Similar to | m/z 422.9 | ¹H NMR (400 MHz, DMSO-d₆) δ 7.88 (d, J = 2.0 Hz, |
| | Example 13 | [M + H₂O] | 1H), 7.42-7.32 (m, 4H), 7.26-7.19 (m, 2H), 4.50 (dd, |
| | | | J = 4.0, 6.8 Hz, 1H), 3.77 (s, 2H), 3.59-3.52 (m, 3H), |
| | | | 3.51-3.46 (m, 3H), 2.92 (t, J = 3.2 Hz, 1H), 2.49 (s, |
| | | | 3H), 2.33 (dt, J = 3.2, 6.8 Hz, 1H), 2.25 (t, J = 6.8 Hz, |
| | | | 2H), 1.96 (dd, J = 3.2, 7.2 Hz, 1H), 1.74 (d, J = 7.6 Hz, |
| | | | 1H), 1.35 (s, 3H). |
| 98 | Similar to | m/z 365.3 | ¹H NMR (400 MHz, DMSO-d₆) δ 8.46 (d, J = 2.0 Hz, |
| | Example 13 | [M + H]⁺ | 1H), 7.37-7.30 (m, 4H), 7.25-7.20 (m, 2H), 4.19- |
| | | | 4.07 (m, 2H), 3.80-3.76 (m, 1H), 3.74-3.70 (m, 1H), |
| | | | 3.27-3.19 (m, 1H), 3.15 (d, J = 3.2 Hz, 1H), 2.70- |
| | | | 2.66 (m, 1H), 2.46 (s, 3H), 1.87-1.67 (m, 3H), 1.50 |
| | | | (d, J = 8.0 Hz, 1H), 1.32 (d, J = 7.2 Hz, 3H). |

### Example 14

### N,8-dimethyl-6-(3-methyl-3-(oxazol-2-yl)398yrrolidine-1-yl)-[1,2,4]triazolo[1,5-a]pyridine-2-carboxamide (Compound 14)

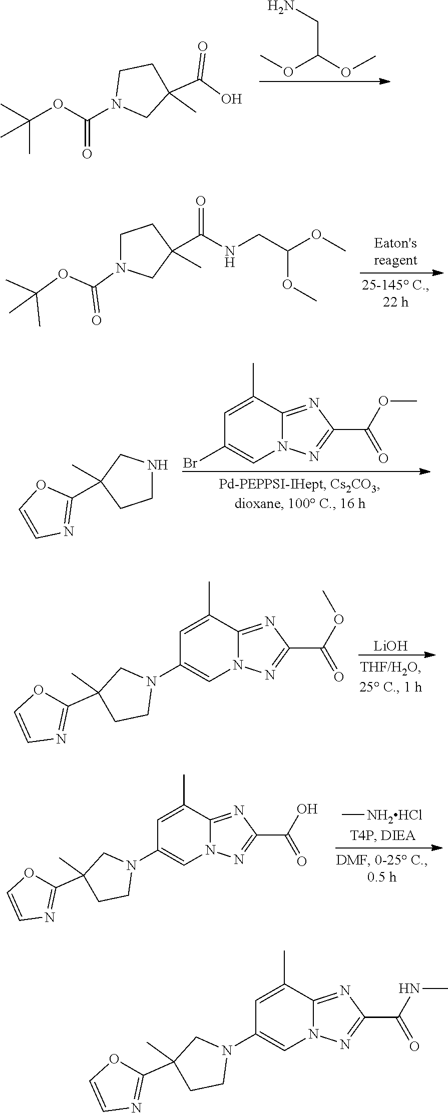

### Step 1: tert-butyl 3-((2,2-dimethoxyethyl)carbamoyl)-3-methylpyrrolidine-1-carboxylate

To a solution of 1-tert-butoxycarbonyl-3-methyl-pyrrolidine-3-carboxylic acid (750 mg, 3.27 mmol, 1 eq) and 2,2-dimethoxyethanamine (378.31 mg, 3.60 mmol, 392.03 μL, 1.1 eq) in DMF (15 mL) was added EDCl (689.81 mg, 3.60 mmol, 1.1 eq), HOBt (486.22 mg, 3.60 mmol, 1.1 eq) in DMF (5 mL) and TEA (662.02 mg, 6.54 mmol, 910.62 μL, 2 eq) at 25° C. for 16 hr. After the mixture was cooled to room temperature, the reaction mixture was diluted with H₂O (50 mL), and then extracted with ethyl acetate (30 mL×3). The combined organic layers were washed with brine (30 mL), dried over Na2SO4, filtered and concentrated under reduced pressure to give a residue. The residue was further purification by pre-HPLC (column: Waters Xbridge Prep OBD C18 150*40 mm*10 um; mobile phase: [water(NH₄HCO3)-ACN]; gradient:16%-46% B over 20 min) and lyophilized. The title compound (1.8 g, 5.69 mmol, 86.96% yield) was obtained as colorless oil.

¹H NMR (400 MHz, DMSO-d6) δ=7.86 (br t, J=5.8 Hz, 1H), 4.35 (t, J=5.6 Hz, 1H), 3.59 (t, J=9.9 Hz, 1H), 3.25 (s, 7H), 3.14 (t, J=5.7 Hz, 2H), 3.03 (d, J=10.8 Hz, 1H), 2.24-2.09 (m, 1H), 1.74-1.64 (m, 1H), 1.38 (s, 9H), 1.20 (s, 3H).

### Step 2: 2-(3-methylpyrrolidin-3-yl)oxazole

A solution of tert-butyl 3-(2,2-dimethoxyethylcarbamoyl)-3-methyl-pyrrolidine-1-carboxylate (500 mg, 1.58 mmol, 1 eq) under argon was added Eaton's reagent (20.90 g, 6.32 mmol, 13.80 mL, 7.2% purity, 4 eq). The mixture was stirred at 145° C. for 6 hr. then heating was stopped and the mixture is allowed to cool to 25° C. for 16 hr. The suspension was filtered and the filtrated was concentrated under reduced pressure. The residue was further purification by pre-HPLC (column: Phenomenex Luna C18 150*25 mm*Oum; mobile phase: [water(FA)-ACN]; gradient:1%-10% B over 9 min) and lyophilized. The title compound (1.8 g, 5.69 mmol, 86.96% yield) was obtained as brown gum.

¹H NMR (400 MHz, DMSO-d6) δ=9.16-8.78 (m, 3H), 8.13 (d, J=0.6 Hz, 1H), 7.20 (s, 1H), 3.45-3.31 (m, 2H), 3.29-3.21 (m, 2H), 2.46-2.38 (m, 1H), 2.11-2.01 (m, 1H), 1.48 (s, 3H).

### Step 3: methyl 8-methyl-6-(3-methyl-3-(oxazol-2-yl)pyrrolidin-1-yl)-[1,2,4]triazolo[1,5-a]pyridine-2-carboxylate

To a mixture of 2-(3-methylpyrrolidin-3-yl)oxazole (100 mg, 657.06 mol, 1 eq) and methyl 6-bromo-8-methyl-[1,2,4]triazolo[1,5-a]pyridine-2-carboxylate (212.95 mg, 788.47 mol, 1.2 eq) in dioxane (8 mL) was added Cs₂CO₃ (428.16 mg, 1.31 mmol, 2 eq) and 1,3-bis[2,6-bis(1-propylbutyl)phenyl]-4,5-dichloro-2H-imidazol-1-ium-2-ide;3-chloropyridine;dichloropalladium (63.92 mg, 65.71 mol, 0.1 eq) in one portion at 25° C. under N₂. The mixture was stirred at 100° C. for 16 hours. After the mixture was cooled to room temperature, the reaction mixture was diluted with H₂O (50 mL), and then extracted with ethyl acetate (30 mL×3). The combined organic layers were washed with brine (30 mL), dried over Na2SO4, filtered and concentrated under reduced pressure to give a residue. The residue was further purification by pre-HPLC (column: YMC-Actus Triart C18 150*30 mm*7 um; mobile phase: [water(FA)-ACN]; gradient:33%-63% B over 10 min) and lyophilized. The title compound (10 mg, 29.15 mol, 2.22% yield, 99.5% purity) was obtained as a white solid.

### Step 4: 8-methyl-6-(3-methyl-3-(oxazol-2-yl)pyrrolidin-1-yl)-[1,2,4]triazolo[1,5-a]pyridine-2-carboxylic acid

To a mixture of methyl 8-methyl-6-(3-methyl-3-oxazol-2-yl-pyrrolidin-1-yl)-[1,2,4]triazolo[1,5-a]pyridine-2-carboxylate (10 mg, 29.29 mol, 1 eq) in THF (0.5 mL) and H2O (0.5 mL) and MeOH (0.5 mL) was added LiOH·H2O (3.69 mg, 87.88 mol, 3 eq) in one portion at 15° C. The mixture was stirred at 15° C. for 1 hours The reaction mixture was concentrated under reduced pressure to give a residue. The title compound (17 mg, crude, Li) was obtained as a white solid.

### Step 5: N,8-dimethyl-6-(3-methyl-3-(oxazol-2-yl)pyrrolidin-1-yl)-[1,2,4]triazolo[1,5-a]pyridine-2-carboxamide

To a mixture of 8-methyl-6-(3-methyl-3-oxazol-2-yl-pyrrolidin-1-yl)-[1,2,4]triazolo[1,5-a]pyridine-2-carboxylic acid (17 mg, 51.93 mol, 1 eq) and methanamine;hydrochloride (17.53 mg, 259.67 mol, 5 eq) in DMF (1 mL) was added DIEA (33.56 mg, 259.67 mol, 45.23 μL, 5 eq) and T4P (56.13 mg, 77.90 mol, 50% purity, 1.5 eq) in one portion at 0° C. The mixture was stirred at 15° C. for 1 hours. After the mixture was cooled to room temperature, the reaction mixture was diluted with H2O (5 mL), and then extracted with ethyl acetate (3 mL×3). The combined organic layers were washed with brine (3 mL), dried over Na2SO4, filtered and concentrated under reduced pressure to give a residue. The residue was further purification by pre-HPLC (column: Waters Xbridge 150*25 mm*Sum; mobile phase: [water(NH₄HCO3)-ACN]; gradient:18%-48% B over 9 min) and lyophilized. The title compound (1.26 mg, 3.66 mol, 7.05% yield, 98.9% purity) was obtained as a white solid.

LC-MS [ESI, M+1]: 340.16.

¹H NMR (400 MHz, DMSO-d6) δ=8.51-8.41 (m, 1H), 8.07 (d, J=0.8 Hz, 1H), 7.86 (d, J=2.0 Hz, 1H), 7.28 (s, 1H), 7.14 (d, J=0.8 Hz, 1H), 3.82 (d, J=9.6 Hz, 1H), 3.50-3.34 (m, 4H), 2.79 (d, J=4.8 Hz, 3H), 2.53 (s, 3H), 2.19-2.11 (m, 1H), 1.51 (s, 3H).

### Example 15

### N,8-dimethyl-6-(3-methyl-3-(thiazol-2-yl)pyrrolidin-1-yl)-[1,2,4]triazolo[1,5-a]pyridine-2-carboxamide (Compound 15)

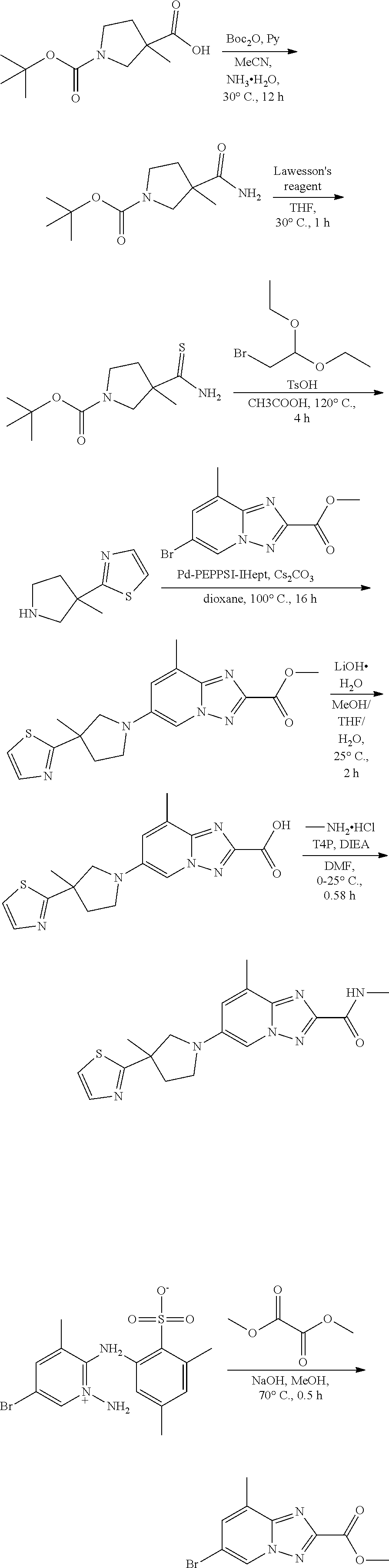

### Step 1: tert-butyl 3-carbamoyl-3-methylpyrrolidine-1-carboxylate

A mixture of 1-(tert-butoxycarbonyl)-3-methylpyrrolidine-3-carboxylic acid (2 g, 8.72 mmol), Boc₂O (2.86 g, 13.08 mmol, 3.01 mL) and Py (3.45 g, 43.62 mmol, 3.52 mL) in ACN (40 mL) was stirred for 0.5 hr. To the mixture was added NH₃·H₂O (2 mL) and the resulted mixture was stirred at 30° C. for 4 hr. The reaction mixture was concentrated under reduced pressure to remove solvent to give the title compound (1.95 g, 97.92% yield) as yellow solid. [3211]¹H NMR (400 MHz, DMSO-d6) δ=7.29 (s, 1H), 6.95 (s, 1H), 3.57 (t, J=10.0 Hz, 1H), 3.29-3.17 (m, 2H), 3.00 (d, J=10.4 Hz, 1H), 2.24-2.05 (m, 1H), 1.66 (m, J=6.6, 12.6 Hz, 1H), 1.39-1.36 (m, 9H), 1.20 (s, 3H)

LC-MS [ESI, M+23]: 251.2.

### Step 2: tert-butyl 3-carbamothioyl-3-methylpyrrolidine-1-carboxylate

To a solution of tert-butyl 3-carbamoyl-3-methylpyrrolidine-1-carboxylate (1.94 g, 8.50 mmol) in THF (20 mL) was added 2,4-bis(4-methoxyphenyl)-2,4-dithioxo-1,3,2,4dithiadiphosphetane (1.79 g, 4.42 mmol). The mixture was stirred at 30° C. for 1 hr. The reaction mixture was concentrated under reduced pressure to remove solvent. The residue was purified by column chromatography (SiO2; Petroleum ether:Ethyl acetate=1:2, Rf=0.65) to give the title compound (1.5 g, 72.24% yield) as yellow oil.

### Step 3: 2-(3-methylpyrrolidin-3-yl)thiazole

To a solution of tert-butyl 3-carbamothioyl-3-methylpyrrolidine-1-carboxylate (200 mg, 818.49 mol), 2-bromo-1,1-diethoxyethane (201.62 mg, 1.02 mmol, 153.91 L) in AcOH (2 mL) was added TsOH (17.62 mg, 102.31 mol). The mixture was stirred at 120° C. for 4 hr. The reaction mixture was concentrated under reduced pressure to remove solvent. The reaction mixture was poured into water (20 mL), extracted with Ethyl acetate (20 mL×2). The water phase was lyophilized. The residue was purified by prep-HPLC (column: Waters Atlantis T3 150*30 mm*5 um; mobile phase: [water(FA)—ACN]; gradient:1%-25% B over 9 min) to give the title compound (60 mg, 43.57% yield) as black-brown oil. [3217]¹H NMR (400 MHz, DMSO-d6) δ=8.33-8.18 (m, 1H), 7.73 (d, J=3.2 Hz, 1H), 7.66 (d, J=3.2 Hz, 1H), 3.15-3.10 (m, 2H), 2.90-2.71 (m, 2H), 2.34-2.21 (m, 2H), 2.04-1.90 (m, 2H), 1.50 (s, 3H)

LC-MS [ESI, M+1]: 169.1.

### Step 4: methyl 8-methyl-6-(3-methyl-3-(thiazol-2-yl) 402 yrrolidine-1-yl)-[1,2,4]triazolo[1,5-a]pyridine-2-carboxylate

To a mixture of 2-(3-methylpyrrolidin-3-yl)thiazole (50 mg, 297.16 mol) and methyl 6-bromopyrazolo[1,5-a]pyridine-2-carboxylate (80.26 mg, 297.16 mol) in dioxane (1 mL) was added Cs₂CO₃ (193.64 mg, 594.32 mol) and 1,3-bis[2,6-bis(1-propylbutyl)phenyl]-4,5-dichloro-2H-imidazol-1-ium-2-ide;3-chloropyridine;dichloropalladium (28.91 mg, 29.72 mol) in one portion at 25° C. under N₂. The mixture was stirred at 100° C. for 16 hr. The reaction mixture was poured into water (10 mL), extracted with ethyl acetate (20 mL×2). The combined organic layers were washed with brine (40 mL), dried over Na2SO4, filtered and concentrated under reduced pressure to give a residue. The residue was purified by prep-HPLC (column: Phenomenex luna C18 150*25 mm*10 um; mobile phase: [water(FA)-ACN]; gradient:33%-63% B over 18 min) to give the title compound (25 mg, 23.54% yield) as yellow solid. [3221]¹H NMR (400 MHz, DMSO-d6) δ=7.94 (d, J=2.0 Hz, 1H), 7.74 (d, J=3.2 Hz, 1H), 7.65 (d, J=3.2 Hz, 1H), 7.33 (s, 1H), 3.89 (s, 3H), 3.86-3.81 (m, 1H), 3.52-3.39 (m, 3H), 3.30 (s, 1H), 2.73 (s, 1H), 2.53 (s, 3H), 2.32-2.16 (m, 1H), 1.58 (s, 3H)

LC-MS [ESI, M+1]: 358.1.

### Step 5: 8-methyl-6-(3-methyl-3-(thiazol-2-yl)₄₀₂yrrolidine-1-yl)-[1,2,4]triazolo[1,5-a]pyridine-2-carboxylic acid

To a solution of methyl 8-methyl-6-(3-methyl-3-(thiazol-2-yl) 402 yrrolidine-1-yl)-[1,2,4]triazolo[1,5-a]pyridine-2-carboxylate (25 mg, 69.94 mol) in THF (0.5 mL), MeOH (0.5 mL), H₂O (0.5 mL) was added LiOH·H₂O (8.81 mg, 209.83 mol). The mixture was stirred at 25° C. for 2 hr. The reaction mixture was concentrated under reduced pressure to remove solvent to give the title compound (24 mg, 99.92% yield) was obtained as yellow solid.

LC-MS [ESI, M+1]: 344.1.

### Step 6: N,8-dimethyl-6-(3-methyl-3-(thiazol-2-yl) 403 yrrolidine-1-yl)-[1,2,4]triazolo[1,5-a]pyridine-2-carboxamide

To a solution of 8-methyl-6-(3-methyl-3-(thiazol-2-yl) 403 yrrolidine-1-yl)-[1,2,4]triazolo[1,5-a]pyridine-2-carboxylic acid (19 mg, 55.33 mol) in DMF (0.5 mL) was added HATU (31.56 mg, 82.99 mol) and DIEA (21.45 mg, 165.99 mol, 28.91 L). The mixture was stirred at 25° C. for 0.08 hr. To the mixture was added methanamine;hydrochloride (3.74 mg, 55.33 mol), the resulted mixture was stirred at 25° C. for 0.5 hr. The residue was purified by prep-HPLC (column: YMC-Actus Triart C18 150*30 mm*7 um; mobile phase: [water(FA)—ACN]; gradient:25%-555% B over 10 min) to give the title compound (4.01 mg, 19.44% yield, 95.6% purity) as white solid. [3228]¹H NMR (400 MHz, CD3OD) δ=7.82 (s, 1H), 7.74-7.70 (m, 1H), 7.51-7.46 (m, 1H), 7.26 (s, 1H), 3.89 (d, J=9.6 Hz, 1H), 3.58-3.47 (m, 3H), 2.97 (s, 3H), 2.60 (s, 4H), 2.37-2.25 (m, 1H), 1.66 (s, 3H)

LC-MS [ESI, M+1]: 357.2.

### Step 7: methyl 6-bromo-8-methyl-[1,2,4]triazolo[1,5-a]pyridine-2-carboxylate

To a solution of 5-bromo-3-methyl-pyridin-1-ium-1,2-diamine;2,4,6-trimethylbenzenesulfonate (30 g, 74.57 mmol, 1 eq) and dimethyl oxalate (17.61 g, 149.14 mmol, 2 eq) in MeOH (1 L) was added NaOH (3.28 g, 82.03 mmol, 1.1 eq). The mixture was stirred at 70° C. for 0.5 h. LCMS showed starting material was consumed and the desired mass was detected. The mixture was concentrated to give a residue. The residue was purified by column chromatography (SiO₂, PE:EtOAc=1:1, Rf=0.43) to give the title compound (8 g, 38% yield) as white solid.

LC-MS [ESI, M+1]: 270.1.

¹H NMR (400 MHz, DMSO-d6) δ=9.32 (d, J=0.8 Hz, 1H), 7.83-7.76 (m, 1H), 3.94 (s, 3H), 2.57 (s, 3H)

| Cmpd. No. | Synthesis | LC/MS | ¹H NMR |
|---|---|---|---|
| 99 | Similar to | m/z 573.3 | ¹H NMR (400 MHz, CD3OD, 297K) δ (ppm) = 8.07-7.90 |
| | Example 15 | [M + H]⁺ | (m, 1H), 7.82 (m, J = 1.4, 19.1 Hz, 1H), 7.72 (m, J = 3.4, |
| | | | 5.4 Hz, 1H), 7.56-7.45 (m, 2H), 7.32-7.17 (m, 2H), 4.26- |
| | | | 4.00 (m, 2H), 3.93-3.66 (m, 7H), 3.57-3.44 (m, 3H), |
| | | | 2.58 (d, J = 16.0 Hz, 4H), 2.39-2.04 (m, 5H), 1.65 (d, J = |
| | | | 7.6 Hz, 3H) |

### Example 16

### N,8-dimethyl-6-(2-methyl-2-phenylcyclobutoxy)-[1,2,4]triazolo[1,5-a]pyridine-2-carboxamide (Compound 16)

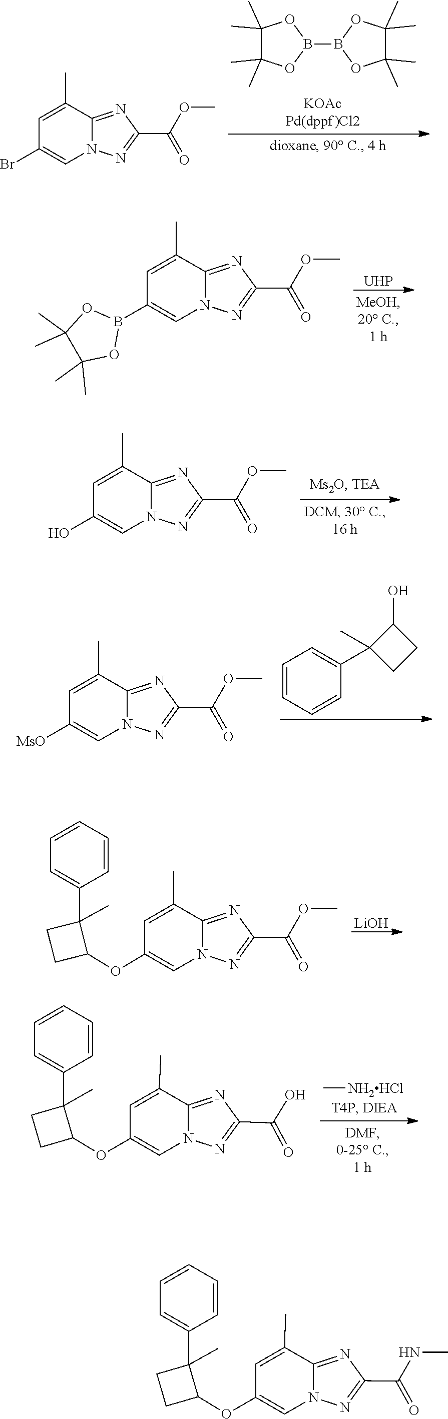

### Step 1: methyl 8-methyl-6-(4,4,5,5-tetramethyl-1,3,2-dioxaborolan-2-yl)-[1,2,4]triazolo[1,5-a]pyridine-2-carboxylate

To a solution of methyl 6-bromo-8-methyl-[1,2,4]triazolo[1,5-a]pyridine-2-carboxylate (2.5 g, 9.26 mmol) and 4,4,5,5-tetramethyl-2-(4,4,5,5-tetramethyl-1,3,2-dioxaborolan-2-yl)-1,3,2-dioxaborolane (3.06 g, 12.03 mmol) in dioxane (110 mL) added KOAc (2.73 g, 27.77 mmol) and Pd(dppf)Cl₂ (338.65 mg, 462.82 mol) under N₂. The mixture was stirred at 90° C. for 4 hours. After the mixture was cooled to room temperature, the reaction mixture was diluted with H₂O (200 mL), and then extracted with ethyl acetate (100 mL×3). The combined organic layers were washed with brine (30 mL), dried over Na2SO4, filtered and concentrated under reduced pressure to give a residue. The residue was purified by column chromatography (SiO2, Petroleum ether/Ethyl acetate=0/1) to give the title compound (4 g, 68.13% yield) as a white solid

¹H NMR (400 MHz, DMSO-d6) δ=8.85 (s, 1H), 7.65 (t, J=1.2 Hz, 1H), 3.92 (s, 3H), 2.57 (s, 3H), 1.33 (s, 12H)

LCMS: m/z 236.1 [M−H]⁺

### Step 2: methyl 6-hydroxy-8-methyl-[1,2,4]triazolo[1,5-a]pyridine-2-carboxylate

To a solution of methyl 8-methyl-6-(4,4,5,5-tetramethyl-1,3,2-dioxaborolan-2-yl)-[1,2,4]triazolo[1,5-a]pyridine-2-carboxylate (2 g, 6.31 mmol, 1 eq) in MeOH (40 mL) was added UHP (1.19 g, 12.61 mmol, 2 eq) at 0° C. The mixture was stirred at 20° C. for 1 hr. The reaction mixture was poured into saturated sodium sulfite solution (30 mL) and adjust to a pH of 4 with hydrochloric acid (1M). The solution was extracted with ethyl acetate (50 mL×3). The solvent was removed under reduce pressure to give a residue. The residue was triturated with methol (10 mL) and filtered to give filter cake. The filter cake was dried under reduced pressure to give a residue. The title compound (580 mg, 2.58 mmol, 40.84% yield, 92% purity) was obtained as a white solid.

LCMS: m/z 208.0 [M−H]+

### Step 3: methyl 8-methyl-6-((methylsulfonyl)oxy)-[1,2,4]triazolo[1,5-a]pyridine-2-carboxylate

To a solution of methyl 6-hydroxy-8-methyl-[1,2,4]triazolo[1,5-a]pyridine-2-carboxylate (200 mg, 965.32 mol, 1 eq) in DCM (3 mL) was added TEA (293.04 mg, 2.90 mmol, 403.08 μL, 3 eq) and the solution of methylsulfonyl methanesulfonate (201.79 mg, 1.16 mmol, 1.2 eq) in DCM (2 mL) at 0° C. The mixture was stirred at 30° C. for 16 hr. The mixture was diluted with 30 mL DCM, washed with brine (10 mL*2), dried over Na2SO4 and filtered. The filtrate was concentrated to give a residue to give the title compound (140 mg, 490.75 mol, 50.84% yield, N/A purity) was obtained as a light yellow solid.

LCMS: m/z 286.0 [M−H]+

### Step 4: methyl 8-methyl-6-(2-methyl-2-phenylcyclobutoxy)-[1,2,4]triazolo[1,5-a]pyridine-2-carboxylate

To a solution of methyl 8-methyl-6-methylsulfonyloxy-[1,2,4]triazolo[1,5-a]pyridine-2-carboxylate (100 mg, 350.54 mol, 1 eq) and 2-methyl-2-phenyl-cyclobutanol (85.30 mg, 525.81 mol, 1.5 eq) in DMF (3 mL) was added Cs₂CO₃ (125.63 mg, 385.59 mol, 1.1 eq). The mixture was stirred at 100° C. for 18 h under N₂. The mixture was quenched by the addition of 50 mL water, extracted with DCM (15 mL*3). The combined organic layers were washed with brine (10 mL*2), dried over Na2SO4 and filtered. The filtrate was concentrated to give a residue. The residue was purified by the prep-TLC(DCM/MeOH=10/1) to give the spot (TLC-plate:DCM/MeOH=10/1, Rf=0.8). The residue was then purified by the prep-HPLC with following conditions: column: Waters Xbridge 150*25 mm*Sum; mobile phase: [water (ammonia hydroxide v/v)-ACN]; gradient:20%-50% B over 10 min. The fractions were evaporated to give methyl 8-methyl-6-(2-methyl-2-phenyl-cyclobutoxy)-[1,2,4]triazolo[1,5-a]pyridine-2-carboxylate (20 mg, 56.92 mol, 16.24% yield, N/A purity) as a white solid.

LCMS: m/z 352.0 [M+H]+

### Step 5: 8-methyl-6-(2-methyl-2-phenylcyclobutoxy)-[1,2,4]triazolo[1,5-a]pyridine-2-carboxylic acid

To a solution of methyl 8-methyl-6-(2-methyl-2-phenyl-cyclobutoxy)-[1,2,4]triazolo[1,5-a]pyridine-2-carboxylate (20 mg, 56.92 mol, 1 eq) in THF (3 mL) was added the solution of LiOH·H₂O (7.17 mg, 170.75 mol, 3 eq) in H₂O (0.3 mL). The mixture was stirred at 20° C. for 16 h under N₂. The mixture was evaporated to give a residue to give the title compound (20 mg, crude) as a white solid.

LCMS: m/z 338.0 [M+H]+

### Step 6: N,8-dimethyl-6-(2-methyl-2-phenylcyclobutoxy)-[1,2,4]triazolo[1,5-a]pyridine-2-carboxamide

To a solution of 8-methyl-6-(2-methyl-2-phenyl-cyclobutoxy)-[1,2,4]triazolo[1,5-a]pyridine-2-carboxylic acid (20 mg, 59.28 mol, 1 eq) in DMF (1.5 mL) was added DIEA (19.15 mg, 148.20 mol, 25.81 μL, 2.5 eq), methanamine;hydrochloride (8.01 mg, 118.56 mol, 2 eq) and T4P (85.43 mg, 118.56 mol, 50% purity, 2 eq) at 0° C. The mixture was stirred at 20° C. for 1 h under N₂. The mixture was quenched by the addition of 15 mL water, extracted with EtOAc(5 mL*3). The combined organic layers were washed with brine (3 mL*2), dried over Na2SO4 and filtered. The filtrate was concentrated to give a residue. The residue was purified by the prep-HPLC with following conditions: column: Waters Xbridge 150*25 mm*Sum; mobile phase: [water (ammonia hydroxide v/v)-ACN]; gradient:15%-45% B over 10 min. The fractions were lyophilized to give N,8-dimethyl-6-(2-methyl-2-phenyl-cyclobutoxy)-[1,2,4]triazolo[1,5-a]pyridine-2-carboxamide (1.15 mg, 3.22 mol, 5.43% yield, 98% purity) as a yellow solid.

LCMS: m/z 351.1 [M+H]+

¹H NMR (400 MHz, DMSO-d6) δ=9.48-9.38 (m, 1H), 7.69-7.61 (m, 1H), 7.04 (d, J=1.2 Hz, 1H), 6.99-6.91 (m, 4H), 6.87-6.81 (m, 1H), 3.12 (s, 3H), 2.49-2.43 (m, 3H), 1.32 (s, 3H), 1.00 (s, 1H), 0.49-0.35 (m, 2H), 0.08-0.10 (m, 2H).

### Example 17

### 6-((5R)-7-(8-methyl-6-(5-(m-tolyl)-2-azaspiro[3.3]heptan-2-yl)-[1,2,4]triazolo[1,5-a]pyridine-2-carbonyl)-2,7-diazaspiro[4.4]nonan-2-yl)picolinic acid (Compound 17)

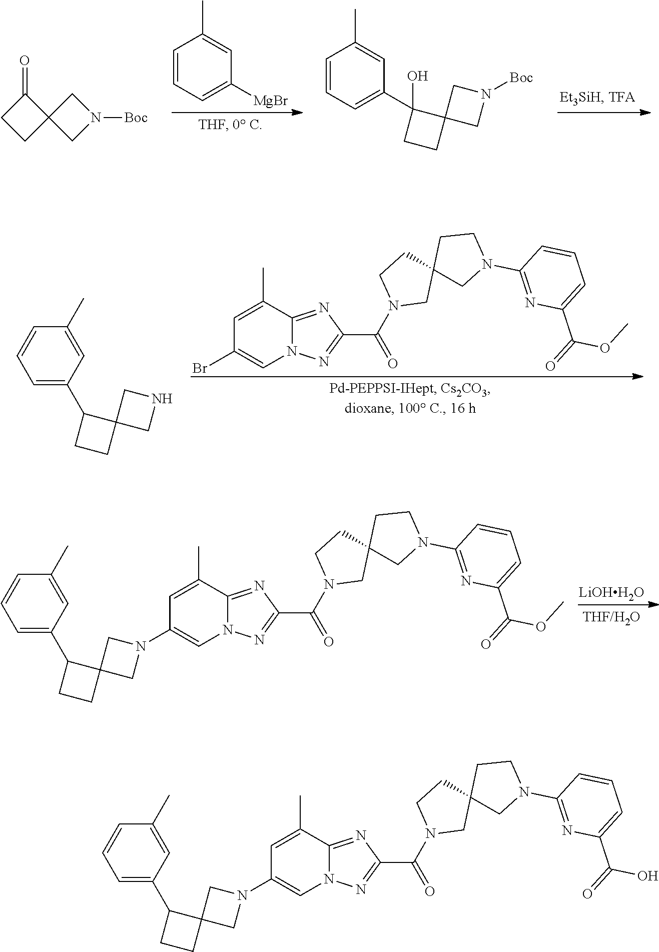

### Step 1: tert-butyl 5-hydroxy-5-(m-tolyl)-2-azaspiro[3.3]heptane-2-carboxylate

A mixture of tert-butyl 7-oxo-2-azaspiro[3.3]heptane-2-carboxylate (200 mg, 946.71 mol, 1 eq) and bromo(m-tolyl)magnesium (1 M, 2.18 mL, 2.3 eq) in THF (2 mL) was degassed and purged with N₂ for 3 times, and then the mixture was stirred at −78° C. for 1 h under N₂ atmosphere. LCMS showed starting material was consumed and one main peak with desired mass was detected. The reaction mixture was extracted with EtOAc (30 mL×2). The combined organic phase was washed with brine (50 mL), dried with anhydrous Na2SO₄, filtered and concentrated in vacuum. The residue was purified by prep-TLC (SiO₂, PE:EtOAc=3:1, Rf=0.30) to give compound tert-butyl 7-hydroxy-7-(m-tolyl)-2-azaspiro[3.3]heptane-2-carboxylate (250 mg, 86% yield) as a colorless oil.

LC-MS [ESI, M+23]: 326.0.

¹H NMR (400 MHz, CHLOROFORM-d) δ=7.33-7.27 (m, 1H), 7.26-7.19 (m, 2H), 7.13 (d, J=7.2 Hz, 1H), 4.48 (d, J=9.2 Hz, 1H), 3.78 (d, J=9.2 Hz, 1H), 3.49 (s, 2H), 2.62 (ddd, J=6.0, 8.8, 12.0 Hz, 1H), 2.39 (s, 3H), 2.36-2.24 (m, 1H), 2.20-2.11 (m, 1H), 2.09 (d, J=10.8 Hz, 1H), 2.04-1.99 (m, 1H), 1.41 (s, 9H).

### Step 2: 5-(m-tolyl)-2-azaspiro[3.3]heptane

To a solution of tert-butyl 7-hydroxy-7-(m-tolyl)-2-azaspiro[3.3]heptane-2-carboxylate (250 mg, 824.01 mol, 1 eq) in DCM (3 mL) was added Et₃SiH (479.07 mg, 4.12 mmol, 658.06 L, 5 eq) and TFA (939.56 mg, 8.24 mmol, 612.09 μL, 10 eq). The mixture was stirred at 0° C. for 1.5 h. LCMS showed starting material was consumed and one main peak with desired mass was detected. The reaction mixture was filtered and the filtrate was concentrated under the reduced pressure to give the product. The residue was purified by prep-HPLC (FA condition column: CD01-Phenomenex luna C18 150*25*10 um; mobile phase: [Water(FA)-MeCN]; gradient:8%-38% B over 10 min). to give compound 7-(m-tolyl)-2-azaspiro[3.3]heptane (70 mg, 45% yield) as a colorless oil.

LC-MS [ESI, M+23]: 188.1.

¹H NMR (400 MHz, CHLOROFORM-d) δ=7.55 (t, J=7.6 Hz, 1H), 7.38 (d, J=7.6 Hz, 1H), 7.29-7.22 (m, 2H), 4.36 (d, J=10.8 Hz, 1H), 4.19 (d, J=10.8 Hz, 1H), 4.00-3.85 (m, 2H), 3.76 (t, J=8.7 Hz, 1H), 2.65 (s, 3H), 2.63-2.55 (m, 2H), 2.51-2.37 (m, 2H).

### Step 3: methyl 6-((5R)-7-(8-methyl-6-(5-(m-tolyl)-2-azaspiro[3.3]heptan-2-yl)-[1,2,4]triazolo[1,5-a]pyridine-2-carbonyl)-2,7-diazaspiro[4.4]nonan-2-yl)picolinate

A mixture of 7-(m-tolyl)-2-azaspiro[3.3]heptane (30.37 mg, 130.17 mol, 1.3 eq, FA), methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (50 mg, 100.13 mol, 1 eq), 1,3-bis[2,6-bis(1-propylbutyl)phenyl]-4,5-dichloro-2H-imidazol-1-ium-2-ide;3-chloropyridine;dichloropalladium (4.87 mg, 5.01 mol, 0.05 eq) and Cs₂CO₃ (97.87 mg, 300.38 mol, 3 eq) in dioxane (1.5 mL) was degassed and purged with N₂ for 3 times, and then the mixture was stirred at 100° C. for 3 h under N₂ atmosphere. LCMS showed starting material was consumed and one main peak with desired mass was detected. The reaction mixture was filtered and the filtrate was concentrated under the reduced pressure to give the product. The residue was purified by prep-HPLC (FA condition) to give compound methyl 6-((5R)-7-(8-methyl-6-(5-(m-tolyl)-2-azaspiro[3.3]heptan-2-yl)-[1,2,4]triazolo[1,5-a]pyridine-2-carbonyl)-2,7-diazaspiro[4.4]nonan-2-yl)picolinate (20 mg, 31% yield) as a yellow oil.

LC-MS [ESI, M+23]: 620.2.

¹H NMR (400 MHz, CHLOROFORM-d) δ=7.59-7.50 (m, 1H), 7.46-7.41 (m, 1H), 7.39-7.32 (m, 1H), 7.22 (q, J=7.2 Hz, 1H), 7.10-6.95 (m, 3H), 6.66 (br d, J=7.2 Hz, 1H), 6.51 (dd, J=8.4, 10.4 Hz, 1H), 4.40 (dq, J=1.2, 7.2 Hz, 2H), 4.13 (br t, J=6.8 Hz, 1H), 3.99-3.84 (m, 5H), 3.71-3.44 (m, 8H), 2.55 (d, J=9.2 Hz, 3H), 2.33 (d, J=5.6 Hz, 3H), 2.26-2.21 (m, 2H), 2.14-2.03 (m, 3H), 2.00 (br s, 1H), 1.76-1.55 (m, 1H), 1.41 (t, J=7.2 Hz, 3H).

### Step 4: 6-((5R)-7-(8-methyl-6-(5-(m-tolyl)-2-azaspiro[3.3]heptan-2-yl)-[1,2,4]triazolo[1,5-a]pyridine-2-carbonyl)-2,7-diazaspiro[4.4]nonan-2-yl)picolinic acid

To a solution of methyl 6-((5R)-7-(8-methyl-6-(5-(m-tolyl)-2-azaspiro[3.3]heptan-2-yl)-[1,2,4]triazolo[1,5-a]pyridine-2-carbonyl)-2,7-diazaspiro[4.4]nonan-2-yl)picolinate (20 mg, 32.27 mol, 1 eq) in THF (0.5 mL) and H₂O (0.15 mL) was added LiOH·H₂O (2.71 mg, 64.54 mol, 2 eq). The mixture was stirred at 25° C. for 1 h. LCMS showed starting material was consumed and the desired mass was detected. The reaction was concentrated under reduced pressure to give a residue. The residue was purified by prep-HPLC (FA condition column: CD01-Phenomenex luna C18 150*25*10 um; mobile phase: [Water(FA)-MeCN]; gradient:37%-67% B over 10 min) to give compound 6-[(5R)-2-[8-methyl-6-[7-(m-tolyl)-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylic acid (11.33 mg, 59% yield) as a white solid.

LC-MS [ESI, M+1]: 592.2.

¹H NMR (400 MHz, CHLOROFORM-d) δ=7.65 (dt, J=5.6, 7.6 Hz, 1H), 7.55-7.38 (m, 2H), 7.22 (q, J=7.2 Hz, 1H), 7.09-6.95 (m, 3H), 6.71-6.52 (m, 2H), 4.24-4.10 (m, 1H), 3.97 (s, 1H), 3.95-3.87 (m, 3H), 3.86-3.72 (m, 1H), 3.69-3.61 (m, 2H), 3.60-3.55 (m, 3H), 3.54-3.43 (m, 2H), 2.56 (d, J=11.2 Hz, 3H), 2.34 (d, J=6.0 Hz, 3H), 2.32-2.16 (m, 4H), 2.16-1.96 (m, 4H).

### Example 18

### (R)-8-methyl-6-(3-methyl-3-phenylpyrrolidin-1-yl)-N-(2-oxaspiro[3.3]heptan-6-yl)-[1,2,4]triazolo[1,5-a]pyridine-2-carboxamide (Compound 18)

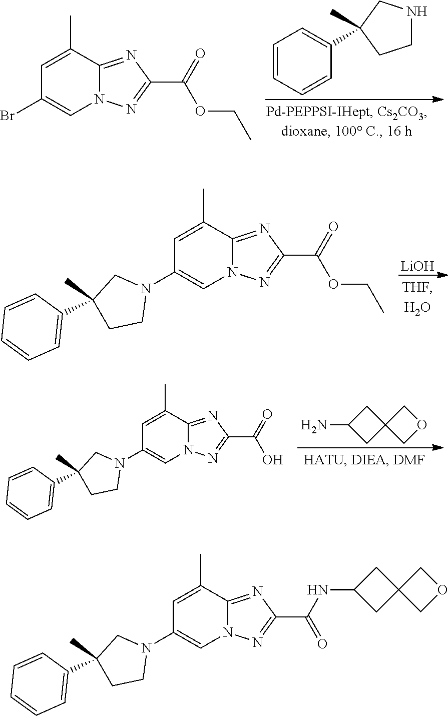

### Step 1: ethyl (R)-8-methyl-6-(3-methyl-3-phenylpyrrolidin-1-yl)-[1,2,4]triazolo[1,5-a]pyridine-2-carboxylate

To a solution of methyl 6-bromo-8-methyl-[1,2,4]triazolo[1,5-a]pyridine-2-carboxylate (1.4 g, 5.18 mmol, 1 eq) and (3R)-3-methyl-3-phenyl-pyrrolidine (835.82 mg, 5.18 mmol, 1 eq) in dioxane (50 mL) was added Cs₂CO₃ (5.07 g, 15.55 mmol, 3 eq) and 1,3-bis[2,6-bis(1-propylbutyl)phenyl]-4,5-dichloro-2H-imidazol-1-ium-2-ide;3-chloropyridine;dichloropalladium (151.27 mg, 155.51 mol, 0.03 eq). The mixture was stirred at 100° C. for 12 h in N₂. LCMS showed starting material was consumed and one main peak with desired mass was detected. The reaction mixture was filtered and concentrated in vacuo. The residue was purified by flash silica gel chromatography (ISCO®; 20 g SepaFlash® Silica Flash Column, Eluent of 30~40% Ethyl acetate/Petroleum ethergradient @80 mL/min) to give methyl 8-methyl-6-[(3R)-3-methyl-3-phenyl-pyrrolidin-1-yl]-[1,2,4]triazolo[1,5-a]pyridine-2-carboxylate (1.3 g, 72% yield) as a yellow oil.

LC-MS [ESI, M+1]: 351.2

¹H NMR (400 MHz, CHLOROFORM-d) δ=7.63 (d, J=1.6 Hz, 1H), 7.35-7.29 (m, 2H), 7.29-7.24 (m, 2H), 7.24-7.18 (m, 1H), 6.98 (s, 1H), 4.04-3.99 (m, 3H), 3.56 (d, J=8.8 Hz, 1H), 3.52-3.42 (m, 2H), 3.40-3.33 (m, 1H), 2.67-2.61 (m, 3H), 2.34 (td, J=8.4, 12.0 Hz, 1H), 2.27-2.16 (m, 1H), 1.42 (s, 3H).

### Step 2: (R)-8-methyl-6-(3-methyl-3-phenylpyrrolidin-1-yl)-[1,2,4]triazolo[1,5-a]pyridine-2-carboxylic acid

To a solution of methyl (R)-8-methyl-6-(3-methyl-3-phenylpyrrolidin-1-yl)-[1,2,4]triazolo[1,5-a]pyridine-2-carboxylate (400 mg, 1.14 mmol, 1 eq) in THF (4 mL) and H₂O (0.5 mL) was added LiOH·H2O (119.75 mg, 2.85 mmol, 2.5 eq). The mixture was stirred at 25° C. for 2 h. LCMS showed starting material was consumed and one main peak with desired mass was detected. The reaction mixture was added HCl (1 M) until pH around 4 and then extracted with EtOAc (10 mL×3). The combined organic phase was washed with brine (10 mL×3), dried over anhydrous Na2SO4, filtered and concentrated in vacuo. to give compound (R)-8-methyl-6-(3-methyl-3-phenylpyrrolidin-1-yl)-[1,2,4]triazolo[1,5-a]pyridine-2-carboxylic acid (350 mg, 1.04 mmol, 91% yield) as a white solid

LC-MS [ESI, M+1]: 377.3.

¹H NMR (400 MHz, CHLOROFORM-d) δ=7.87 (s, 1H), 7.41-7.35 (m, 2H), 7.35-7.31 (m, 2H), 7.25 (br s, 1H), 7.07 (s, 1H), 3.65 (d, J=9.2 Hz, 1H), 3.61-3.51 (m, 2H), 3.48-3.42 (m, 1H), 2.71 (s, 3H), 2.41 (td, J=8.4, 12.0 Hz, 1H), 2.33-2.24 (m, 1H), 1.48 (s, 3H).

### Step 3: (R)-8-methyl-6-(3-methyl-3-phenylpyrrolidin-1-yl)-N-(2-oxaspiro[3.3]heptan-6-yl)-[1,2,4]triazolo[1,5-a]pyridine-2-carboxamide

To a solution of 2-oxaspiro[3.3]heptan-6-amine (8.41 mg, 74.32 mol, 1 eq) and 8-methyl-6-[(3R)-3-methyl-3-phenyl-pyrrolidin-1-yl]-[1,2,4]triazolo[1,5-a]pyridine-2-carboxylic acid (25 mg, 74.32 mol, 1 eq) in DMF (1 mL) was added HATU (56.52 mg, 148.64 mol, 2 eq) and DIEA (28.82 mg, 222.96 mol, 38.84 μL, 3 eq). The mixture was stirred at 25° C. for 1 h. LCMS showed starting material was consumed and one main peak with desired mass was detected. The reaction was filtered to give a residue. The residue was purified by prep-HPLC (column: Phenomenex luna C18 150×25 mm×10 um; mobile phase: [water(FA)-ACN]; gradient:40%-70% B over 10 min). to give compound 8-methyl-6-[(3R)-3-methyl-3-phenyl-pyrrolidin-1-yl]-N-(2-oxaspiro[3.3]heptan-6-yl)-[1,2,4]triazolo[1,5-a]pyridine-2-carboxamide (18.68 mg, 57% yield) as a white solid.

LC-MS [ESI, M+1]: 432.2.

¹H NMR (400 MHz, CHLOROFORM-d) δ=7.66 (br s, 1H), 7.42 (br d, J=7.6 Hz, 1H), 7.40-7.34 (i, 2H), 7.34-7.29 (m, 2H), 7.27-7.22 (m, 1H), 7.00 (br s, 1H), 4.78 (s, 2H), 4.66 (s, 2H), 4.56-4.42 (m, 1H), 3.61 (br d, J=8.8 Hz, 1H), 3.57-3.46 (, 2H), 3.41 (dt, J=3.6, 8.4 Hz, 1H), 2.87-2.74 (i, 2H), 2.62 (s, 3H), 2.45-2.34 (, 1H), 2.33-2.21 (m, 3H), 1.47 (s, 3H)

| Cmpd. No. | Synth. | LC/MS | ¹H NMR |
|---|---|---|---|
| 100 | Similar to | m/z 408.2 | ¹H NMR (400 MHz, CHLOROFORM-d) δ = 7.73 (br t, |
| | Example | [M + H]⁺ | J = 5.8 Hz, 1H), 7.66 (s, 1H), 7.40-7.34 (m, 2H), 7.34- |
| | 18 | | 7.30 (m, 2H), 7.25 (br s, 1H), 7.02 (br s, 1H), 3.61 (d, J = |
| | | | 8.9 Hz, 1H), 3.58-3.51 (m, 3H), 3.51-3.47 (m, 1H), |
| | | | 3.41 (dt, J = 3.4, 8.6 Hz, 1H), 2.63 (s, 3H), 2.43-2.34 |
| | | | (m, 1H), 2.30-2.23 (m, 1H), 1.47 (s, 3H), 1.32 (s, 6H) |
| 101 | Similar to | m/z 405.3 | ¹H NMR (400 MHz, CHLOROFORM-d) δ = 8.83 (br d, |
| | Example | [M + H]⁺ | J = 6.6 Hz, 1H), 8.63 (br s, 1H), 7.67 (s, 1H), 7.41-7.33 |
| | 18 | | (m, 2H), 7.33-7.23 (m, 3H), 6.98 (s, 1H), 4.99 (br d, J = |
| | | | 1.5 Hz, 1H), 3.67-3.46 (m, 6H), 3.45-3.34 (m, 2H), |
| | | | 2.61 (s, 3H), 2.48-2.33 (m, 2H), 2.27 (br dd, J = 3.9, 6.8 |
| | | | Hz, 2H), 1.46 (s, 3H) |
| 102 | Similar to | m/z 405.3 | ¹H NMR (400 MHz, DMSO-d6) δ = 9.34 (br d, J = 5.4 |
| | Example | [M + H]⁺ | Hz, 1H), 9.15 (br d, J = 1.8 Hz, 1H), 8.88 (d, J = 7.4 Hz, |
| | 18 | | 1H), 7.88 (d, J = 1.6 Hz, 1H), 7.43-7.31 (m, 5H), 7.26- |
| | | | 7.21 (m, 1H), 4.69-4.56 (m, 1H), 3.62-3.58 (m, 1H), |
| | | | 3.55-3.46 (m, 4H), 3.23 (dt, J = 6.4, 11.5 Hz, 3H), 2.56 |
| | | | (s, 3H), 2.30-2.16 (m, 3H), 2.07-1.98 (m, 1H), 1.36 (s, |
| | | | 3H) |
| 103 | Similar to | m/z 419.3 | ¹H NMR (400 MHz, DMSO-d6) δ = 9.88-9.28 (m, 2H), |
| | Example | [M + H]⁺ | 7.92 (br s, 1H), 7.44-7.28 (m, 5H), 7.27-7.19 (m, 1H), |
| | 18 | | 5.12-5.03 (m, 1H), 4.84-4.78 (m, 1H), 3.63-3.57 (m, |
| | | | 1H), 3.55-3.47 (m, 2H), 3.45-3.28 (m, 3H), 3.21 (br s, |
| | | | 2H), 3.09-2.93 (m, 3H), 2.53 (br s, 3H), 2.33-2.05 (m, |
| | | | 4H), 1.35 (s, 3H) |
| 104 | Similar to | m/z 488.2 | ¹H NMR (400 MHz, CHLOROFORM-d) δ = 7.89 (br d, |
| | Example | [M + H]⁺ | J = 5.4 Hz, 1H), 7.65 (br s, 1H), 7.40-7.34 (m, 2H), |
| | 18 | | 7.34-7.30 (m, 2H), 7.28 (br s, 1H), 7.02 (br s, 1H), |
| | | | 4.61-4.48 (m, 1H), 4.01-3.78 (m, 1H), 3.60 (br d, J = |
| | | | 8.5 Hz, 1H), 3.57-3.46 (m, 2H), 3.45-3.36 (m, 1H), |
| | | | 2.62 (s, 3H), 2.47 (br dd, J = 9.3, 14.8 Hz, 1H), 2.42- |
| | | | 2.34 (m, 1H), 2.32-2.18 (m, 3H), 2.09-1.98 (m, 3H), |
| | | | 1.47 (s, 3H) |
| 105 | Similar to | m/z 420.3 | ¹H NMR (400 MHz, CHLOROFORM-d) δ = 7.60 (s, |
| | Example | [M + H]⁺ | 1H), 7.34-7.30 (m, 2H), 7.28-7.24 (m, 2H), 7.22-7.17 |
| | 18 | | (m, 1H), 6.96 (s, 1H), 4.75-4.60 (m, 1H), 4.50-4.40 |
| | | | (m, 1H), 3.54 (d, J = 8.9 Hz, 1H), 3.50-3.40 (m, 2H), |
| | | | 3.35 (dt, J = 3.4, 8.6 Hz, 1H), 2.57 (s, 3H), 2.37-2.32 |
| | | | (m, 1H), 2.24-2.13 (m, 3H), 2.11-2.03 (m, 1H), 1.81 |
| | | | (td, J = 6.9, 13.8 Hz, 1H), 1.69-1.60 (m, 1H), 1.59-1.51 |
| | | | (m, 1H), 1.41 (s, 3H) |
| 106 | Similar to | m/z 422.2 | ¹H NMR (400 MHz, CHLOROFORM-d) δ = 7.91 (br s, |
| | Example | [M + H]⁺ | 1H), 7.64 (br s, 1H), 7.43-7.34 (m, 2H), 7.34-7.29 |
| | 18 | | (m, 2H), 7.27-7.23 (m, 1H), 7.02 (s, 1H), 5.20-4.92 |
| | | | (m, 1H), 4.68 (br d, J = 6.6 Hz, 2H), 4.53 (br d, J = 6.5 |
| | | | Hz, 2H), 3.98 (br d, J = 5.4 Hz, 2H), 3.61 (br d, J = 9.0 |
| | | | Hz, 1H), 3.56-3.46 (m, 2H), 3.41 (br d, J = 2.5 Hz, |
| | | | 1H), 2.63 (br s, 3H), 2.42-2.36 (m, 1H), 2.27 (br d, J = |
| | | | 4.6 Hz, 1H), 1.47 (s, 3H) |
| 107 | Similar to | m/z 422.2 | ¹H NMR (400 MHz, CHLOROFORM-d) δ = 7.80-7.48 |
| | Example | [M + H]⁺ | (m, 2H), 7.40-7.35 (m, 2H), 7.34-7.30 (m, 2H), 7.25 |
| | 18 | | (br s, 1H), 7.00 (br d, J = 5.1 Hz, 1H), 3.63-3.39 (m, |
| | | | 6H), 3.28 (s, 3H), 2.79-2.52 (m, 3H), 2.43-2.34 (m, |
| | | | 1H), 2.31-2.22 (m, 1H), 1.47 (s, 3H), 1.24 (br s, 6H) |
| 108 | Similar to | m/z 477.3 | ¹H NMR (400 MHz, CHLOROFORM-d) δ = 7.57 (s, |
| | Example | [M + H]⁺ | 1H), 7.53-7.45 (m, 1H), 7.35-7.29 (m, 2H), 7.28- |
| | 18 | | 7.24 (m, 2H), 7.22-7.18 (m, 1H), 6.95 (s, 1H), 4.57- |
| | | | 4.46 (m, 1H), 4.44-4.31 (m, 1H), 4.02 (br dd, J = 7.3, |
| | | | 13.0 Hz, 1H), 3.85-3.75 (m, 1H), 3.63-3.53 (m, 2H), |
| | | | 3.52-3.40 (m, 3H), 3.39-3.31 (m, 1H), 2.56 (s, 3H), |
| | | | 2.37-2.29 (m, 2H), 2.27-2.16 (m, 2H), 1.41 (s, 3H), |
| | | | 1.13 (dt, J = 2.8, 7.4 Hz, 3H) |
| 109 | Similar to | m/z 454.1 | ¹H NMR (400 MHz, CHLOROFORM-d) δ = 7.76 (br d, |
| | Example | [M + H]⁺ | J = 7.9 Hz, 1H), 7.65 (s, 1H), 7.41-7.35 (m, 2H), 7.34- |
| | 18 | | 7.30 (m, 2H), 7.27-7.23 (m, 1H), 7.03 (s, 1H), 5.04 |
| | | | (sxt, J = 7.0 Hz, 1H), 3.62 (d, J = 8.9 Hz, 1H), 3.59 (s, |
| | | | 2H), 3.49 (br d, J = 9.0 Hz, 1H), 3.42 (dt, J = 4.0, 8.8 |
| | | | Hz, 1H), 3.35 (br dd, J = 6.7, 13.6 Hz, 1H), 3.23-3.13 |
| | | | (m, 2H), 2.75-2.66 (m, 1H), 2.64 (s, 3H), 2.49-2.36 |
| | | | (m, 2H), 2.32-2.22 (m, 1H), 1.47 (s, 3H) |
| 110 | Similar to | m/z 479.3 | ¹H NMR (400 MHz, DMSO-d₆) δ = 8.56 (br d, J = 7.9 |
| | Example | [M + H]⁺ | Hz, 1H), 8.26 (s, 1H), 7.90 (s, 1H), 7.42-7.37 (m, 2H), |
| | 18 | | 7.37-7.29 (m, 3H), 7.26-7.21 (m, 1H), 4.29-4.21 (m, |
| | | | 2H), 3.60 (br d, J = 9.5 Hz, 1H), 3.53-3.47 (m, 2H), |
| | | | 3.37 (br dd, J = 3.4, 5.8 Hz, 1H), 3.32 (s, 2H), 3.25- |
| | | | 3.19 (m, 2H), 2.87 (br dd, J = 4.1, 9.9 Hz, 1H), 2.70 (br |
| | | | dd, J = 2.9, 10.2 Hz, 1H), 2.56 (s, 3H), 2.26 (br t, J = |
| | | | 7.0 Hz, 2H), 1.35 (s, 3H) |
| 111 | Similar to | m/z 432.2 | ¹H NMR (400 MHz, CHLOROFORM-d) δ = 7.67 (s, |
| | Example | [M + H]⁺ | 1H), 7.61 (s, 1H), 7.41-7.34 (m, 2H), 7.34-7.29 (m, |
| | 18 | | 2H), 7.27-7.23 (m, 1H), 7.00 (s, 1H), 3.60 (d, J = 8.9 |
| | | | Hz, 1H), 3.56-3.46 (m, 2H), 3.41 (dt, J = 3.6, 8.7 Hz, |
| | | | 1H), 2.62 (s, 3H), 2.38 (td, J = 8.3, 12.1 Hz, 1H), 2.29- |
| | | | 2.25 (m, 1H), 2.22 (br s, 2H), 2.08 (br d, J = 5.8 Hz, |
| | | | 2H), 2.00 (dd, J = 1.4, 3.3 Hz, 2H), 1.86-1.80 (m, 2H), |
| | | | 1.46 (s, 3H) |
| 112 | Similar to | m/z 474.1 | ¹H NMR (400 MHz, CHLOROFORM-d) δ = 7.61 (s, |
| | Example | [M + H]⁺ | 1H), 7.49 (br d, J = 7.1 Hz, 1H), 7.35-7.29 (m, 2H), |
| | 18 | | 7.28-7.24 (m, 2H), 7.22-7.18 (m, 1H), 6.95 (s, 1H), |
| | | | 4.60-4.48 (m, 1H), 3.55 (d, J = 8.9 Hz, 1H), 3.51- |
| | | | 3.41 (m, 2H), 3.35 (dt, J = 3.5, 8.7 Hz, 1H), 3.05 (quin, |
| | | | J = 8.4 Hz, 1H), 2.65-2.52 (m, 4H), 2.50-2.43 (m, |
| | | | 1H), 2.41-2.26 (m, 4H), 2.25-2.15 (m, 2H), 2.10- |
| | | | 1.98 (m, 2H), 1.41 (s, 3H) |
| 113 | Similar to | m/z 432.1 | ¹H NMR (400 MHz, CHLOROFORM-d) δ = 7.66 (br d, |
| | Example | [M + H]⁺ | J = 12.6 Hz, 1H), 7.41-7.35 (m, 2H), 7.35-7.30 (m, |
| | 18 | | 2H), 7.25 (br s, 1H), 7.02 (br d, J = 6.3 Hz, 1H), 4.76 |
| | | | (d, J = 6.1 Hz, 1H), 4.65 (td, J = 6.3, 12.8 Hz, 3H), 4.29 |
| | | | (s, 1H), 4.05 (t, J = 6.9 Hz, 1H), 3.97 (s, 1H), 3.76 (t, J = |
| | | | 7.1 Hz, 1H), 3.64-3.58 (m, 1H), 3.57-3.47 (m, 2H), |
| | | | 3.46-3.39 (m, 1H), 2.65 (d, J = 10.3 Hz, 3H), 2.42- |
| | | | 2.34 (m, 1H), 2.33-2.21 (m, 3H), 1.47 (d, J = 2.9 Hz, |
| | | | 3H) |
| 114 | Similar to | m/z 419.4 | ¹H NMR (400 MHz, DMSO-d6) δ = 9.82-9.31 (m, 2H), |
| | Example | [M + H]⁺ | 7.91 (br s, 1H), 7.43-7.28 (m, 4H), 7.27-7.19 (m, 1H), |
| | 18 | | 5.06 (br dd, J = 7.9, 16.0 Hz, 1H), 4.81 (td, J = 8.0, 16.2 |
| | | | Hz, 1H), 3.59 (br d, J = 9.5 Hz, 2H), 3.54-3.46 (m, 2H), |
| | | | 3.43-3.30 (m, 3H), 3.27-3.15 (m, 2H), 3.10-2.93 (m, |
| | | | 3H), 2.55-2.48 (m, 3H), 2.28-2.04 (m, 4H), 1.47-1.20 |
| | | | (m, 3H) |
| 115 | Similar to | m/z 422.2 | ¹H NMR (CHLOROFORM-d, 400 MHz): δ = 7.60 (br |
| | Example | [M + H]⁺ | s, 1.0H), 7.32-7.39 (m, 2.1H), 7.27-7.31 (m, 2.0H), 7.20- |
| | 18 | | 7.27 (m, 1.0H), 6.97 (br s, 0.9H), 4.37 (br s, 2.1H), 4.22- |
| | | | 4.30 (m, 1.0H), 4.05 (br d, J = 9.2 Hz, 1.2H), 3.76-3.90 |
| | | | (m, 4.2H), 3.55 (br d, J = 8.8 Hz, 1.1H), 3.42-3.52 (m, |
| | | | 2.0H), 3.31-3.41 (m, 1.0H), 2.58 (br s, 3.0H), 2.28-2.38 |
| | | | (m, 1.0H), 2.17-2.27 (m, 1.0H), 1.42 ppm (s, 3.0H) |
| 116 | Similar to | m/z 450.2 | ¹H NMR (CHLOROFORM-d, 400 MHz): δ = 7.69 (br |
| | Example | [M + H]⁺ | t, J = 5.6 Hz, 1.0H), 7.59 (s, 1.0H), 7.28-7.36 (m, 2.0H), |
| | 18 | | 7.17-7.28 (m, 3.0H), 6.95 (s, 1.0H), 3.69-3.81 (m, |
| | | | 4.0H), 3.49-3.57 (m, 3.0H), 3.40-3.49 (m, 2.1H), 3.35 |
| | | | (td, J = 8.4, 3.4 Hz, 1.1H), 2.57 (s, 3.0H), 2.28-2.37 (m, |
| | | | 1.2H), 2.17-2.25 (m, 1.1H), 1.64-1.75 (m, 2.1H), 1.55- |
| | | | 1.63 (m, 2.0H), 1.41 ppm (s, 3.0H) |
| 117 | Similar to | m/z 421.3 | ¹H NMR (DMSO-d₆, 400 MHz): δ = 8.78 (br d, J = 7.2 |
| | Example | [M + H]⁺ | Hz, 1.0H), 8.38 (s, 1.1H), 7.86 (s, 1.0H), 7.29-7.41 (m, |
| | 18 | | 5.0H), 7.20-7.26 (m, 1.0H), 4.27 (br s, 2.1H), 3.56-3.61 |
| | | | (m, 1.1H), 3.43-3.53 (m, 3.1H), 3.36 (br d, J = 9.2 Hz, |
| | | | 2.0H), 3.18 (br d, J = 11.2 Hz, 1.0H), 3.00 (br d, J = 12.0 |
| | | | Hz, 1.0H), 2.55 (s, 3.0H), 2.20-2.31 (m, 2.0H), 1.35 |
| | | | ppm (s, 3.0H) |
| 118 | Similar to | m/z 406.1 | ¹H NMR (CHLOROFORM-d, 400 MHz): δ = 7.57 (s, |
| | Example | [M + H]⁺ | 1.0H), 7.47 (br d, J = 7.2 Hz, 0.9H), 7.26-7.34 (m, 2.1H), |
| | 18 | | 7.11-7.26 (m, 2.9H), 6.92 (s, 0.9H), 4.73 (br d, J = 3.6 |
| | | | Hz, 1.0H), 3.96 (br d, J = 7.6 Hz, 1.1H), 3.85 (br d, |
| | | | J = 5.6 Hz, 1.1H), 3.77 (br s, 2.0H), 3.52 (br d, J = 8.8 Hz, |
| | | | 1.0H), 3.37-3.48 (m, 2.0H), 3.32 (td, J = 8.8, 3.3 z, |
| | | | 1.0H), 2.54 H (s, 3.0H), 2.25-2.34 (m, 2.1H), 2.15-2.22 |
| | | | (m, 1.1H), 1.88-1.98 (m, 1.0H), 1.38 ppm (s, 3.0H) |
| 119 | Similar to | m/z 465.2 | ¹H NMR (DMSO-d₆, 400 MHz): δ = 8.41 (br s, 0.9H), |
| | Example | [M + H]⁺ | 8.16 (s, 0.3H), 7.90 (s, 0.9H), 7.29-7.48 (m, 5.0H), |
| | 18 | | 7.20-7.27 (m, 1.0H), 5.00 (br s, 2.0H), 3.89 (br s, 2.0H), |
| | | | 3.60 (br d, J = 9.6 Hz, 2.0H), 3.52 (br d, J = 9.4 Hz, |
| | | | 4.0H), 3.41 (br d, J = 5.6 Hz, 2.0H), 2.95 (br dd, J = 9.2, |
| | | | 5.1 Hz, 2.0H), 2.69 (br d, J = 6.8 Hz, 2.1H), 2.55 (s, |
| | | | 3.2H), 2.26 (br t, J = 6.8 Hz, 2.0H), 1.36 ppm (s, 3.0H) |
| 120 | Similar to | m/z 406.1 | ¹H NMR (CHLOROFORM-d, 400 MHz): δ = 7.90 (br |
| | Example | [M + H]⁺ | s, 0.9H), 7.63 (br s, 1.0H), 7.28-7.41 (m, 4.0H), 7.26 (br |
| | 18 | | s, 1.1H), 7.00 (br s, 1.0H), 3.65 (br d, J = 5.6 Hz, 2.1H), |
| | | | 3.56 (br d, J = 8.8 Hz, 1.3H), 3.44-3.52 (m, 2.1H), 3.38 |
| | | | (td, J = 8.3, 3.1 Hz, 1.2H), 2.61 (s, 3.4H), 2.30-2.37 (m, |
| | | | 1.4H), 2.19-2.28 (m, 1.4H), 1.37-1.52 (m, 4.0H), 0.83- |
| | | | 0.94 (m, 2.1H), 0.64-0.74 ppm (m, 2.1H) |
| 121 | Similar to | m/z 424.3 | ¹H NMR (CHLOROFORM-d, 400 MHz): δ = 7.57 (s, |
| | Example | [M + H]⁺ | 0.9H), 7.28 (br d, J = 7.2 Hz, 2.9H), 7.21-7.25 (m, 1.9H), |
| | 18 | | 6.95 (s, 0.9H), 5.07-5.29 (m, 0.9H), 4.63-4.76 (m, |
| | | | 0.9H), 4.02-4.17 (m, 2.0H), 3.85-4.02 (m, 2.0H), 3.53 |
| | | | (d, J = 8.8 Hz, 0.9H), 3.38-3.49 (m, 2.0H), 3.33 (td, |
| | | | J = 8.8, 3.8 Hz, 1.0H), 2.54 (s, 3.0H), 2.31 (dt, J = 12.4, |
| | | | 8.3 Hz, 1.0H), 2.14-2.24 (m, 1.0H), 1.38 ppm (s, 3.1H) |
| 122 | Similar to | m/z 432.1 | ¹H NMR (CHLOROFORM-d, 400 MHz): δ = 7.62 (s, |
| | Example | [M + H]⁺ | 0.9H), 7.46 (br t, J = 5.6 Hz, 1.1H), 7.30-7.37 (m, 2.0H), |
| | 18 | | 7.20-7.30 (m, 3.1H), 6.99 (s, 0.9H), 4.52 (s, 0.9H), 3.83 |
| | | | (d, J = 6.4 Hz, 2.1H), 3.65 (s, 2.0H), 3.57 (br d, J = 8.8 |
| | | | Hz, 1.0H), 3.42-3.53 (m, 2.1H), 3.33-3.42 (m, 1.0H), |
| | | | 2.60 (s, 3.0H), 2.31-2.37 (m, 1.1H), 2.24 (td, J = 7.6, 3.4 |
| | | | Hz, 1.1H), 1.76 (br d, J = 4.4 Hz, 2.1H), 1.59 (br d, |
| | | | J = 4.8 Hz, 2.1H), 1.43 ppm (s, 3.0H) |
| 123 | Similar to | m/z 476.2 | ¹H NMR (CHLOROFORM-d, 400 MHz): δ = 7.76 (s, |
| | Example | [M + H]⁺ | 0.9H), 7.66 (s, 1.0H), 7.37 (br d, J = 7.2 Hz, 2.0H), 7.33 |
| | 18 | | (s, 2.0H), 7.25 (br s, 1.0H), 7.01 (s, 1.0H), 4.04 (s, |
| | | | 2.0H), 3.60 (s, 1.0H), 3.47-3.57 (m, 2.0H), 3.38-3.45 |
| | | | (m, 1.0H), 2.63 (s, 3.0H), 2.39 (br d, J = 12.0 Hz, 1.1H), |
| | | | 2.25-2.30 (m, 1.0H), 2.23 (br d, J = 4.4 Hz, 2.0H), 2.10 |
| | | | (br d, J = 4.4 Hz, 2.0H), 1.47 (s, 3.0H), 1.30 ppm (s, |
| | | | 6.1H) |
| 124 | Similar to | m/z 418.2 | ¹H NMR (CHLOROFORM-d, 400 MHz): δ = 7.66 (br |
| | Example | [M + H]⁺ | d, J = 6.4 Hz, 1.0H), 7.38 (br d, J = 7.2 Hz, 2.0H), 7.30- |
| | 18 | | 7.35 (m, 2.0H), 7.29 (br d, J = 2.8 Hz, 1.2H), 7.02 (br s, |
| | | | 0.9H), 5.84 (br s, 0.5H), 5.23 (br s, 0.5H), 4.72 (br d, |
| | | | J = 12.4 Hz, 1.0H), 4.00-4.16 (m, 2.0H), 3.93 (br dd, |
| | | | J = 19.3, 7.6 Hz, 1.1H), 3.73 (br d, J = 8.0 Hz, 1.0H), 3.63 |
| | | | (br d, J = 8.8 Hz, 1.0H), 3.47-3.59 (m, 2.1H), 3.43 (br s, |
| | | | 1.0H), 2.66 (br s, 3.0H), 2.40 (br d, J = 10.4 Hz, 1.2H), |
| | | | 2.29 (br d, J = 5.6 Hz, 1.1H), 1.98 (br s, 2.2H), 1.49 ppm |
| | | | (s, 3.0H) |
| 125 | Similar to | m/z 462.3 | ¹H NMR (CHLOROFORM-d, 400 MHz): δ = 7.64 (br |
| | Example | [M + H]⁺ | d, J = 4.8 Hz, 1.0H), 7.28-7.40 (m, 4.0H), 7.22-7.26 (m, |
| | 18 | | 1.1H), 6.98 (s, 1.0H), 4.39 (d, J = 12.0 Hz, 0.6H), 4.19 |
| | | | (d, J = 12.4 Hz, 0.5H), 4.06-4.16 (m, 1.0H), 3.90 (d, |
| | | | J = 12.0 Hz, 0.6H), 3.78-3.86 (m, 1.1H), 3.72 (d, J = 8.8 |
| | | | Hz, 2.9H), 3.64 (d, J = 12.6 Hz, 0.5H), 3.60 (br d, J = 8.8 |
| | | | Hz, 1.1H), 3.46-3.54 (m, 2.0H), 3.37-3.44 (m, 1.1H), |
| | | | 2.64 (d, J = 4.0 Hz, 3.1H), 2.33-2.51 (m, 2.2H), 2.20- |
| | | | 2.30 (m, 1.1H), 1.82-1.93 (m, 1.1H), 1.46 (s, 3.1H), |
| | | | 1.40 ppm (d, J = 16.8 Hz, 3.2H) |
| 126 | Similar to | m/z 448.1 | ¹H NMR (CHLOROFORM-d, 400 MHz): δ = 7.67 (s, |
| | Example | [M + H]⁺ | 1.0H), 7.36 (br d, J = 7.2 Hz, 2.1H), 7.29-7.33 (m, 2.0H), |
| | 18 | | 7.24 (br s, 1.0H), 6.99 (br s, 1.0H), 4.46 (br d, J = 12.0 |
| | | | Hz, 0.6H), 4.27 (br d, J = 12.8 Hz, 0.5H), 4.11 (br t, |
| | | | J = 6.4 Hz, 1.0H), 3.86 (s, 1.9H), 3.60-3.65 (m, 1.2H), |
| | | | 3.46-3.55 (m, 2.3H), 3.41 (td, J = 8.4, 3.3 Hz, 1.3H), |
| | | | 2.64 (s, 3.0H), 2.51 (br d, J = 5.6 Hz, 1.3H), 2.38 (br d, |
| | | | J = 12.0 Hz, 1.2H), 2.21-2.30 (m, 1.1H), 1.89 (br dd, |
| | | | J = 12.8, 6.6 Hz, 1.1H), 1.40-1.53 ppm (m, 6.2H) |
| 127 | Similar to | m/z 443.2 | ¹H NMR (400 MHz, DMSO-d₆) δ = 9.44 (s, 1H), 7.91 |
| | Example | [M + H]⁺ | (s, 1H), 7.43-7.31 (m, 5H), 7.28-7.19 (m, 1H), 3.88 |
| | 18 | | (s, 2H), 3.64-3.57 (m, 1H), 3.56-3.46 (m, 2H), 3.39 |
| | | | (br s, 1H), 2.75-2.68 (m, 2H), 2.56 (s, 3H), 2.33-2.23 |
| | | | (m, 4H), 1.36 (s, 3H) |
| 128 | Similar to | m/z 392.4 | ¹H NMR (400 MHz, CHLOROFORM-d) δ = 7.63 (s, |
| | Example | [M + H]⁺ | 1H), 7.40-7.34 (m, 2H), 7.34-7.29 (m, 2H), 7.27- |
| | 18 | | 7.23 (m, 1H), 6.99 (s, 1H), 4.96 (br dd, J = 6.4, 10.8 Hz, |
| | | | 1H), 4.82-4.71 (m, 1H), 4.62-4.46 (m, 2H), 4.13 (br |
| | | | dd, J = 4.0, 11.2 Hz, 1H), 3.59 (d, J = 8.8 Hz, 1H), 3.56- |
| | | | 3.45 (m, 2H), 3.40 (dt, J = 3.6, 8.8 Hz, 1H), 2.62 (s, 3H), |
| | | | 2.37 (td, J = 8.4, 12.0 Hz, 1H), 2.30-2.21 (m, 1H), 1.46 |
| | | | (s, 3H) |
| 129 | Similar to | m/z 418.3 | ¹H NMR (400 MHz, CHLOROFORM-d) δ = 7.66 (s, |
| | Example | [M + H]⁺ | 1H), 7.41 (br s, 1H), 7.39-7.34 (m, 2H), 7.33-7.29 |
| | 18 | | (m, 2H), 7.27-7.23 (m, 1H), 7.03 (s, 1H), 4.08 (d, J = |
| | | | 8.4 Hz, 2H), 3.77 (br d, J = 8.4 Hz, 2H), 3.60 (d, J = 8.8 |
| | | | Hz, 1H), 3.57-3.46 (m, 2H), 3.41 (dt, J = 3.6, 8.8 Hz, |
| | | | 1H), 2.81 (br s, 1H), 2.62 (s, 3H), 2.39 (td, J = 8.4, 12.0 |
| | | | Hz, 1H), 2.30-2.22 (m, 1H), 1.97 (s, 2H), 1.46 (s, 3H) |
| 130 | Similar to | m/z 422.2 | ¹H NMR (400 MHz, CHLOROFORM-d) δ = 7.91 (br s, |
| | Example | [M + H]⁺ | 1H), 7.67 (s, 1H), 7.38 (d, J = 7.2 Hz, 2H), 7.34-7.29 |
| | 18 | | (m, 2H), 7.28 (br s, 1H), 7.01 (s, 1H), 3.69 (q, J = 6.4 |
| | | | Hz, 2H), 3.60 (d, J = 8.8 Hz, 1H), 3.57-3.45 (m, 2H), |
| | | | 3.41 (dt, J = 3.6, 8.4 Hz, 1H), 2.63 (s, 3H), 2.37 (s, 1H), |
| | | | 2.31-2.22 (m, 1H), 1.87-1.84 (m, 2H), 1.47 (s, 3H), |
| | | | 1.34 (s, 6H) |
| 131 | Similar to | m/z 450.2 | ¹H NMR (400 MHz, CHLOROFORM-d) δ = 7.66 (br s, |
| | Example | [M + H]⁺ | 1H), 7.49 (br s, 1H), 7.42-7.35 (m, 2H), 7.35-7.29 |
| | 18 | | (m, 2H), 7.26 (br s, 1H), 7.04 (br s, 1H), 3.96-3.67 (m, |
| | | | 6H), 3.65-3.37 (m, 4H), 2.65 (s, 3H), 2.42-2.36 (m, |
| | | | 1H), 2.32-2.25 (m, 1H), 2.12 (br d, J = 14.0 Hz, 2H), |
| | | | 1.94 (br t, J = 9.2 Hz, 2H), 1.48 (s, 3H) |
| 132 | Similar to | m/z 418.2 | ¹H NMR (400 MHz, CHLOROFORM-d) δ = 7.94 (br s, |
| | Example | [M + H]⁺ | 1H), 7.67 (br s, 1H), 7.42-7.33 (m, 2H), 7.33-7.29 |
| | 18 | | (m, 2H), 7.26 (br d, J = 7.6 Hz, 1H), 7.08 (br s, 1H), |
| | | | 3.65-3.45 (m, 3H), 3.40 (br s, 1H), 2.61 (s, 3H), 2.45- |
| | | | 2.20 (m, 8H), 1.45 (s, 3H) |
| 133 | Similar to | m/z 487.4 | ¹H NMR (400 MHz, DMSO-d₆) δ = 11.14-10.79 (m, |
| | Example | [M + H]⁺ | 1H), 10.01 (br s, 1H), 8.98 (d, J = 7.6 Hz, 1H), 7.88 (d, |
| | 18 | | J = 1.6 Hz, 1H), 7.42-7.31 (m, 5H), 7.26-7.20 (m, |
| | | | 1H), 5.17 (br t, J = 8.8 Hz, 1H), 4.90-4.78 (m, 1H), |
| | | | 3.56-3.33 (m, 6H), 2.90 (td, J = 7.6, 13.2 Hz, 1H), |
| | | | 2.59-2.52 (m, 3H), 2.48-2.43 (m, 1H), 2.42 (s, 3H), |
| | | | 2.25 (br t, J = 7.2 Hz, 2H), 1.35 (s, 3H) |
| 134 | Similar to | m/z 449.4 | ¹H NMR (400 MHz, DMSO-d₆) δ = 9.46 (br s, 2H), |
| | Example | [M + H]⁺ | 9.01 (d, J = 8.0 Hz, 1H), 7.89 (s, 1H), 7.42-7.32 (m, |
| | 18 | | 5H), 7.28-7.19 (m, 1H), 4.90-4.78 (m, 1H), 3.60 (br |
| | | | d, J = 9.6 Hz, 2H), 3.55-3.45 (m, 4H), 3.31-3.19 (m, |
| | | | 3H), 2.57 (s, 3H), 2.26 (br t, J = 7.2 Hz, 2H), 1.36 (s, |
| | | | 3H) |
| 135 | Similar to | m/z 444.9 | ¹H NMR (400 MHz, CHLOROFORM-d) δ = 7.65 (br |
| | Example | [M + H]⁺ | s, 1H), 7.41-7.29 (m, 4H), 7.27-7.22 (m, 1H), 7.01 |
| | 18 | | (br s, 1H), 6.41-6.26 (m, 1H), 5.53 (br t, J = 6.8 Hz, |
| | | | 1H), 4.94 (br t, J = 6.4 Hz, 1H), 4.56-4.46 (m, 1H), |
| | | | 4.45-4.32 (m, 1H), 3.97 (dt, J = 6.4, 11.2 Hz, 1H), |
| | | | 3.67-3.58 (m, 1H), 3.57-3.46 (m, 2H), 3.46-3.36 (m, |
| | | | 1H), 2.96-2.81 (m, 1H), 2.70 (br d, J = 18.4 Hz, 1H), |
| | | | 2.66-2.59 (m, 3H), 2.51-2.44 (m, 1H), 2.42-2.32 (m, |
| | | | 1H), 2.31-2.22 (m, 1H), 2.10-2.01 (m, 2H), 1.47 (s, |
| | | | 3H) |
| 136 | Similar to | m/z 435.9 | ¹H NMR (400 MHz, CHLOROFORM-d) δ = 8.11 (br |
| | Example | [M + H]⁺ | t, J = 6.4 Hz, 1H), 7.69 (d, J = 1.6 Hz, 1H), 7.40-7.33 |
| | 18 | | (m, 2H), 7.33-7.28 (m, 2H), 7.27-7.23 (m, 1H), 7.01 |
| | | | (s, 1H), 3.72 (d, J = 6.4 Hz, 2H), 3.59 (d, J = 8.8 Hz, |
| | | | 1H), 3.55-3.45 (m, 2H), 3.40 (dt, J = 3.6, 8.8 Hz, 1H), |
| | | | 2.61 (s, 3H), 2.37 (td, J = 8.4, 12.4 Hz, 1H), 2.30-2.21 |
| | | | (m, 1H), 1.46 (s, 3H), 1.35 (s, 6H) |
| 137 | Similar to | m/z 336.0 | ¹H NMR (400 MHz, CHLOROFORM-d) δ = 7.65 (s, |
| | Example | [M + H]⁺ | 1H), 7.41-7.35 (m, 2H), 7.35-7.30 (m, 2H), 7.29 (s, |
| | 18 | | 1H), 7.17 (br s, 1H), 7.02 (s, 1H), 5.71 (br s, 1H), 3.62 |
| | | | (d, J = 8.8 Hz, 1H), 3.58-3.47 (m, 2H), 3.42 (dt, J = |
| | | | 3.6, 8.8 Hz, 1H), 2.64 (s, 3H), 2.39 (td, J = 8.4, 12.4 |
| | | | Hz, 1H), 2.32-2.23 (m, 1H), 1.48 (s, 3H) |
| 138 | Similar to | m/z 438.3 | ¹H NMR (400 MHz, CHLOROFORM-d) δ = 7.70 (br t, |
| | Example | [M + H]+ | J = 5.6 Hz, 1H), 7.60 (s, 1H), 7.35-7.28 (m, 2H), 7.28- |
| | 18 | | 7.24 (m, 2H), 7.23-7.21 (m, 1H), 6.95 (s, 1H), 3.64- |
| | | | 3.50 (m, 3H), 3.49-3.40 (m, 2H), 3.40-3.27 (m, 6H), |
| | | | 2.58 (s, 3H), 2.38-2.28 (m, 1H), 2.22 (br dd, J = 4.0, |
| | | | 7.3 Hz, 1H), 1.41 (s, 3H), 1.20 (s, 3H) |
| 139 | Similar to | m/z 394.1 | ¹H NMR (400 MHz, CHLOROFORM-d) δ = 7.63 (br s, |
| | Example | [M + H]+ | 1H), 7.51 (br d, J = 7.0 Hz, 1H), 7.40-7.33 (m, 2H), |
| | 18 | | 7.33-7.29 (m, 2H), 7.27-7.23 (m, 1H), 7.00 (s, 1H), |
| | | | 4.33 (br dd, J = 3.6, 5.9 Hz, 1H), 3.83 (br dd, J = 3.2, |
| | | | 11.1 Hz, 1H), 3.70 (br dd, J = 6.2, 11.2 Hz, 1H), 3.57 |
| | | | (br d, J = 8.9 Hz, 1H), 3.54-3.44 (m, 2H), 3.43-3.34 |
| | | | (m, 1H), 2.60 (s, 3H), 2.42-2.31 (m, 1H), 2.30-2.20 |
| | | | (m, 1H), 1.45 (s, 3H), 1.32 (d, J = 6.8 Hz, 3H) |
| 140 | Similar to | m/z 435.1 | ¹H NMR (400 MHz, CHLOROFORM-d) δ = 9.20- |
| | Example | [M + H]+ | 9.01 (m, 1H), 8.71 (br s, 1H), 7.66 (s, 1H), 7.39-7.33 |
| | 18 | | (m, 2H), 7.33-7.28 (m, 2H), 7.26-7.21 (m, 1H), 6.98 |
| | | | (br s, 1H), 4.87 (br s, 1H), 4.13-4.01 (m, 1H), 3.74- |
| | | | 3.55 (m, 4H), 3.54-3.45 (m, 5H), 3.42-3.30 (m, 2H), |
| | | | 2.61 (s, 3H), 2.42-2.31 (m, 1H), 2.30-2.19 (m, 1H), |
| | | | 1.45 (s, 3H) |
| 141 | Similar to | m/z 436.2 | ¹H NMR (400 MHz, CHLOROFORM-d) δ = 7.77- |
| | Example | [M + H]+ | 7.59 (m, 1H), 7.48 (br s, 1H), 7.41-7.34 (m, 2H), 7.34- |
| | 18 | | 7.29 (m, 2H), 7.27-7.23 (m, 1H), 7.17-6.97 (m, 1H), |
| | | | 4.69-4.55 (m, 1H), 4.40 (br t, J = 7.1 Hz, 1H), 3.93- |
| | | | 3.87 (m, 1H), 3.86-3.75 (m, 2H), 3.62 (br d, J = 8.8 |
| | | | Hz, 1H), 3.58-3.47 (m, 2H), 3.46-3.38 (m, 1H), 2.52 |
| | | | (br s, 3H), 2.44-2.35 (m, 1H), 2.30-2.23 (m, 1H), |
| | | | 1.47 (s, 3H), 1.40 (br s, 3H) |
| 142 | Similar to | m/z 420.3 | ¹H NMR (400 MHz, CHLOROFORM-d) δ = 7.66 (s, |
| | Example | [M + H]+ | 2H), 7.41-7.35 (m, 2H), 7.34-7.30 (m, 2H), 7.27- |
| | 18 | | 7.23 (m, 1H), 7.03 (s, 1H), 4.37 (br s, 2H), 3.60 (br d, J = |
| | | | 8.4 Hz, 1H), 3.57-3.46 (m, 2H), 3.45-3.37 (m, 1H), |
| | | | 2.64 (s, 3H), 2.43-2.34 (m, 1H), 2.30-2.23 (m, 1H), |
| | | | 2.15-2.08 (m, 1H), 2.03-1.87 (m, 3H), 1.80-1.77 (m, |
| | | | 2H), 1.47 (s, 3H) |
| 143 | Similar to | m/z 462.1 | ¹H NMR (400 MHz, CHLOROFORM-d) δ = 7.65 (s, |
| | Example | [M + H]+ | 1H), 7.40-7.34 (m, 2H), 7.34-7.29 (m, 2H), 7.25 (br |
| | 18 | | s, 1H), 7.00 (s, 1H), 4.29 (br dd, J = 6.8, 11.7 Hz, 1H), |
| | | | 4.23-4.15 (m, 1H), 4.03 (dd, J = 7.6, 12.5 Hz, 1H), |
| | | | 3.98-3.84 (m, 1H), 3.70 (d, J = 8.4 Hz, 3H), 3.63- |
| | | | 3.32 (m, 5H), 2.74-2.59 (m, 4H), 2.57-2.44 (m, 2H), |
| | | | 2.43-2.33 (m, 1H), 2.31-2.13 (m, 2H), 1.76-1.70 (m, |
| | | | 1H), 1.47 (s, 3H) |
| 144 | Similar to | m/z 483.3 | ¹H NMR (400 MHz, CHLOROFORM-d) δ = 7.65 (s, |
| | Example | [M + H]+ | 1H), 7.41-7.34 (m, 2H), 7.34-7.29 (m, 2H), 7.29- |
| | 18 | | 7.27 (m, 1H), 7.03 (s, 1H), 4.18 (br s, 2H), 3.97 (br s, |
| | | | 2H), 3.61 (d, J = 8.8 Hz, 1H), 3.56-3.47 (m, 2H), 3.43 |
| | | | (br dd, J = 3.6, 8.6 Hz, 1H), 3.40-3.29 (m, 4H), 2.81 |
| | | | (s, 3H), 2.63 (s, 3H), 2.39 (td, J = 8.4, 12.1 Hz, 1H), |
| | | | 2.32-2.23 (m, 1H), 1.47 (s, 3H) |
| 145 | Similar to | m/z 462.2 | ¹H NMR (400 MHz, CHLOROFORM-d) δ = 7.75 (d, J = |
| | Example | [M + H]⁺ | 4.8 Hz, 1H), 7.60 (s, 1H), 7.27 (s, 5H), 6.95 (s, 1H), |
| | 18 | | 4.87-4.70 (m, 1H), 4.06 (dd, J = 9.6, 6.4 Hz, 1H), 3.87- |
| | | | 3.72 (m, 3H), 3.68 (d, J = 10.4 Hz, 1H), 3.62-3.52 |
| | | | (m, 2H), 3.50-3.40 (m, 2H), 3.36 (s, 1H), 2.65-2.60 |
| | | | (m, 1H), 2.56 (s, 3H), 2.41-2.27 (m, 2H), 2.21 (d, J = |
| | | | 3.6 Hz, 1H), 2.05-1.94 (m, 2H), 1.41 (s, 3H). |
| 146 | Similar to | m/z 418.2 | ¹H NMR (400 MHz, CHLOROFORM-d) δ = 7.67 (d, J = |
| | Example | [M + H]⁺ | 3.2 Hz, 1H), 7.40-7.34 (m, 2H), 7.34-7.29 (m, 2H), |
| | 18 | | 7.27 (s, 1H), 7.03 (s, 1H), 5.59 (s, 1H), 4.94 (s, 1H), |
| | | | 3.97 (s, 1H), 3.63-3.56 (m, 2H), 3.55-3.46 (m, 2H), |
| | | | 3.45-3.37 (m, 1H), 2.65 (s, 3H), 2.42-2.34 (m, 2H), |
| | | | 2.32-2.22 (m, 2H), 2.00 (s, 1H), 1.91 (d, J = 4.0 Hz, |
| | | | 1H), 1.46 (s, 3H) |
| 147 | Similar to | m/z 453.2 | ¹H NMR (400 MHz, CHLOROFORM-d) δ = 8.17 (d, J = |
| | Example | [M + H]⁺ | 8.0 Hz, 1H), 7.85 (d, J = 8.0 Hz, 1H), 7.65 (s, 1H), |
| | 18 | | 7.47-7.34 (m, 2H), 7.34-7.29 (m, 2H), 7.27 (s, 1H), |
| | | | 7.01 (s, 1H), 5.20-5.02 (m, 1H), 3.65-3.55 (m, 2H), |
| | | | 3.55-3.46 (m, 2H), 3.45-3.36 (m, 2H), 3.33-3.22 (m, |
| | | | 2H), 2.75-2.66 (m, 1H), 2.63 (s, 3H), 2.49 (dd, J = |
| | | | 12.8, 6.4 Hz, 1H), 2.41-2.34 (m, 1H), 2.27 (d, J = 4.0 |
| | | | Hz, 1H), 1.47 (s, 3H). |
| 148 | Similar to | m/z 424.1 | ¹H NMR (400 MHz, CHLOROFORM-d) δ = 7.71 (br t, |
| | Example | [M + H]+ | J = 6.2 Hz, 1H), 7.66 (d, J = 2.0 Hz, 1H), 7.40-7.35 |
| | 18 | | (m, 2H), 7.34-7.30 (m, 2H), 7.27-7.23 (m, 1H), 7.03 |
| | | | (s, 1H), 3.89-3.81 (m, 1H), 3.63 (br s, 3H), 3.61 (d, J = |
| | | | 9.2 Hz, 1H), 3.57 (s, 3H), 3.49-3.47 (m, 3H), 3.45- |
| | | | 3.39 (m, 1H), 2.63 (s, 3H), 2.42-2.35 (m, 1H), 2.30- |
| | | | 2.23 (m, 1H), 1.47 (s, 3H) |
| 149 | Similar to | m/z 423.3 | ¹H NMR (400 MHz, DMSO-d6) δ = 10.07-9.70 (m, |
| | Example | [M + H]+ | 2H), 8.79 (d, J = 7.6 Hz, 1H), 7.91 (d, J = 1.6 Hz, 1H), |
| | 18 | | 7.42-7.32 (m, 5H), 7.26-7.21 (m, 1H), 5.44-5.25 (m, |
| | | | 1H), 4.71-4.61 (m, 1H), 3.64-3.56 (m, 2H), 3.56- |
| | | | 3.44 (m, 5H), 3.42-3.34 (m, 1H), 2.57 (s, 3H), 2.26 (br |
| | | | t, J = 7.2 Hz, 2H), 1.36 (s, 3H) |
| 150 | Similar to | m/z 441.3 | ¹H NMR (400 MHz, DMSO-d6) δ = 10.17 (br s, 2H), |
| | Example | [M + H]+ | 9.11 (d, J = 9.2 Hz, 1H), 7.91 (d, J = 1.6 Hz, 1H), 7.42- |
| | 18 | | 7.32 (m, 5H), 7.26-7.20 (m, 1H), 5.08 (td, J = 9.2, |
| | | | 12.0 Hz, 1H), 3.88-3.71 (m, 3H), 3.68-3.56 (m, 3H), |
| | | | 3.49-3.46 (m, 1H), 3.41-3.34 (m, 1H), 2.57 (s, 3H), |
| | | | 2.26 (br t, J = 7.2 Hz, 2H), 1.35 (s, 3H) |
| 151 | Similar to | m/z 422.3 | ¹H NMR (400 MHz, CHLOROFORM-d) δ = 7.65 (br s, |
| | Example | [M + H]+ | 1H), 7.55 (br s, 1H), 7.40-7.34 (m, 2H), 7.34-7.29 |
| | 18 | | (m, 2H), 7.27-7.23 (m, 1H), 7.02 (br s, 1H), 4.48 (br d, |
| | | | J = 17.2 Hz, 2H), 4.31-4.11 (m, 2H), 3.90 (br d, J = |
| | | | 9.2 Hz, 1H), 3.78 (br d, J = 9.2 Hz, 1H), 3.61 (br d, J = |
| | | | 7.8 Hz, 1H), 3.58-3.46 (m, 2H), 3.42 (br s, 1H), 2.63 |
| | | | (br s, 3H), 2.43-2.34 (m, 1H), 2.28 (br s, 1H), 1.47 (s, |
| | | | 3H) |
| 152 | Similar to | m/z 447.2 | ¹H NMR (400 MHz, DMSO-d6) δ = 8.70 (br d, J = 6.8 |
| | Example | [M + H]+ | Hz, 1H), 8.38 (br s, 1H), 7.88 (s, 1H), 7.44-7.28 (m, |
| | 18 | | 5H), 7.26-7.21 (m, 1H), 4.53-4.46 (m, 1H), 3.97 (br |
| | | | d, J = 8.4 Hz, 1H), 3.90 (br s, 2H), 3.82 (br s, 2H), 3.73- |
| | | | 3.70 (m, 1H), 3.62-3.58 (m, 1H), 3.54-3.47 (m, 2H), |
| | | | 3.40-3.34 (m, 1H), 2.55 (s, 3H), 2.46 (br s, 1H), 2.35 |
| | | | (br dd, J = 5.6, 13.1 Hz, 1H), 2.26 (br t, J = 6.8 Hz, |
| | | | 2H), 1.35 (s, 3H) |
| 153 | Similar to | m/z 408.2 | ¹H NMR (400 MHz, CHLOROFORM-d) δ = 7.59 (s, |
| | Example | [M + H]+ | 1H), 7.37 (s, 1H), 7.35-7.29 (m, 2H), 7.27 (br d, J = |
| | 18 | | 1.6 Hz, 2H), 7.22-7.18 (m, 1H), 6.97 (s, 1H), 3.70 (s, |
| | | | 2H), 3.54 (d, J = 8.8 Hz, 1H), 3.51-3.40 (m, 2H), 3.36 |
| | | | (dt, J = 3.6, 8.8 Hz, 1H), 2.57 (s, 3H), 2.33 (td, J = 8.4, |
| | | | 12.2 Hz, 1H), 2.25-2.17 (m, 1H), 1.47-1.31 (m, 9H) |
| 154 | Similar to | m/z 440.2 | ¹H NMR (400 MHz, CHLOROFORM-d) δ = 7.60 (s, |
| | Example | [M + H]+ | 1H), 7.40 (br d, J = 8.0 Hz, 1H), 7.35-7.28 (m, 2H), |
| | 18 | | 7.28-7.24 (m, 2H), 7.23-7.18 (m, 1H), 6.96 (s, 1H), |
| | | | 4.75-4.58 (m, 1H), 3.55 (d, J = 8.8 Hz, 1H), 3.52- |
| | | | 3.40 (m, 2H), 3.36 (dt, J = 3.6, 8.7 Hz, 1H), 2.58 (s, |
| | | | 3H), 2.39-2.25 (m, 3H), 2.25-2.15 (m, 2H), 2.14- |
| | | | 2.01 (m, 2H), 1.89-1.77 (m, 1H), 1.41 (s, 3H) |
| 155 | Similar to | m/z 432.2 | ¹H NMR (400 MHz, CHLOROFORM-d) δ = 7.68 (s, |
| | Example | [M + H]+ | 1H), 7.60 (d, J = 1.6 Hz, 1H), 7.35-7.29 (m, 2H), 7.27 |
| | 18 | | (s, 2H), 7.22-7.17 (m, 1H), 6.94 (s, 1H), 3.71 (s, 2H), |
| | | | 3.55 (d, J = 8.8 Hz, 1H), 3.51-3.41 (m, 2H), 3.36 (dt, J = |
| | | | 3.6, 8.7 Hz, 1H), 2.56 (s, 3H), 2.33 (td, J = 8.4, 12.1 |
| | | | Hz, 1H), 2.24-2.17 (m, 1H), 2.12 (s, 6H), 1.41 (s, 3H) |
| 156 | Similar to | m/z 505.4 | ¹H NMR (400 MHz, CHLOROFORM-d) δ ppm 1.47 |
| | Example | [M + H]⁺ | (s, 3 H) 1.95-2.09 (m, 4 H) 2.20-2.45 (m, 4 H) 2.59- |
| | 18 | | 2.66 (m, 3 H) 3.07 (br d, J = 4.8 Hz, 2 H) 3.27 (br d, |
| | | | J = 6.4 Hz, 1 H) 3.40-3.53 (m, 5 H) 3.60 (br d, J = 8.8 |
| | | | Hz, 1 H) 4.41 (br s, 2 H) 4.63-4.80 (m, 1 H) 6.99 (s, 1 |
| | | | H) 7.24 (br s, 1 H) 7.30-7.39 (m, 4 H) 7.65 (s, 1 H) |
| | | | 8.56 (br d, J = 7.2 Hz, 2 H) |
| 157 | Similar to | m/z 436.2 | ¹H NMR (400 MHz, CHLOROFORM-d) δ ppm 1.46 |
| | Example | [M + H]⁺ | (s, 3 H) 1.74 (br d, J = 8.8 Hz, 1 H) 2.11 (br d, J = 11.2 |
| | 18 | | Hz, 1 H) 2.22-2.29 (m, 1 H) 2.33-2.42 (m, 1 H) 2.61 |
| | | | (br s, 3 H) 3.36-3.62 (m, 6 H) 3.85-4.01 (m, 2 H) |
| | | | 4.07 (br s, 1 H) 4.17 (br d, J = 10.0 Hz, 1 H) 6.99 (br s, 1 |
| | | | H) 7.23-7.27 (m, 1 H) 7.29-7.33 (m, 2 H) 7.34-7.41 |
| | | | (m, 2 H) 7.46-7.72 (m, 2 H) |
| 158 | Similar to | m/z 446.3 | ¹H NMR (400 MHz, CHLOROFORM-d) δ = 7.68 (s, |
| | Example | [M + H]⁺ | 1H), 7.43-7.36 (m, 2H), 7.35-7.28 (m, 3H), 7.06 (s, |
| | 18 | | 1H), 4.52 (br d, J = 12.4 Hz, 1H), 4.28 (br d, J = 12.8 |
| | | | Hz, 1H), 4.12-3.99 (m, 1H), 3.75 (br dd, J = 3.6, 12.8 |
| | | | Hz, 1H), 3.66-3.40 (m, 4H), 2.66 (s, 3H), 2.46-2.36 |
| | | | (m, 1H), 2.34-2.25 (m, 3H), 1.67 (br s, 1H), 1.48 (s, |
| | | | 3H) |
| 159 | Similar to | m/z 419.9 | ¹H NMR (400 MHz, CHLOROFORM-d) δ = 8.23 (s, |
| | Example | [M + H]+ | 1H), 7.58 (br s, 1H), 7.35-7.29 (m, 2H), 7.28-7.24 |
| | 18 | | (m, 2H), 7.22-7.17 (m, 1H), 6.93 (br s, 1H), 4.23- |
| | | | 4.01 (m, 1H), 3.94-3.71 (m, 2H), 3.65-3.27 (m, 5H), |
| | | | 2.57 (d, J = 4.0 Hz, 3H), 2.37-2.27 (m, 1H), 2.25- |
| | | | 2.17 (m, 1H), 2.06-1.83 (m, 2H), 1.50-1.32 (m, 6H) |
| 160 | Similar to | m/z 405.9 | ¹H NMR (400 MHz, CHLOROFORM-d) δ = 8 (br s, |
| | Example | [M + H]+ | 2H), 7.42-7.35 (m, 2H), 7.35-7.31 (m, 2H), 7.28- |
| | 18 | | 7.25 (m, 1H), 7.02 (br s, 1H), 4.29-4.07 (m, 2H), 3.67- |
| | | | 3.34 (m, 4H), 2.63 (s, 3H), 2.45-2.33 (m, 1H), 2.32- |
| | | | 2.15 (m, 3H), 1.87-1.75 (m, 1H), 1.64-1.39 (m, 4H) |
| 161 | Similar to | m/z 405.9 | ¹H NMR (400 MHz, CHLOROFORM-d) δ = 8 (br s, |
| | Example | [M + H]+ | 1H), 7.63 (br s, 1H), 7.41-7.27 (m, 5H), 7.01 (br s, |
| | 18 | | 1H), 4.65 (br s, 2H), 3.65-3.35 (m, 4H), 2.63 (br s, |
| | | | 3H), 2.35 (br d, J = 8 Hz, 2H), 2.26 (br s, 2H), 2.04 (br |
| | | | dd, J = 8, 9.7 Hz, 2H), 1.47 (s, 3H) |
| 162 | Similar to | m/z 463.2 | ¹H NMR (400 MHz, CHLOROFORM-d) δ = 8.54- |
| | Example | [M]⁺ | 8.32 (m, 2H), 7.52 (s, 1H), 7.33-7.12 (m, 5H), 6.85 (s, |
| | 18 | | 1H), 4.79 (br d, J = 5.2 Hz, 1H), 3.66-3.56 (m, 1H), |
| | | | 3.42 (br s, 3H), 3.37 (br t, J = 9.2 Hz, 2H), 3.31-3.23 |
| | | | (m, 1H), 2.56-2.34 (m, 4H), 2.32-2.10 (m, 3H), 1.35 |
| | | | (s, 6H), 1.20 (s, 3H) |
| 163 | Similar to | m/z 420.1 | ¹H NMR (400 MHz, CHLOROFORM-d) δ = 8.00 (br s, |
| | Example | [M]⁺ | 1H), 7.65 (br s, 1H), 7.40-7.33 (m, 2H), 7.33-7.29 |
| | 18 | | (m, 2H), 7.26-7.20 (m, 1H), 7.01 (br s, 1H), 3.59 (br d, |
| | | | J = 8.8 Hz, 1H), 3.54-3.45 (m, 2H), 3.43-3.36 (m, |
| | | | 1H), 2.59 (s, 3H), 2.43-2.32 (m, 1H), 2.30-2.20 (m, |
| | | | 1H), 1.73 (br s, 2H), 1.50-1.32 (m, 5H) |
| 164 | Similar to | m/z 434.0 | ¹H NMR (400 MHz, CHLOROFORM-d) δ = 7.67 (br s, |
| | Example | [M + H]⁺ | 1H), 7.40-7.29 (m, 4H), 7.27-7.23 (m, 1H), 6.99 (s, |
| | 18 | | 1H), 3.59 (br d, J = 8.8 Hz, 1H), 3.53-3.46 (m, 2H), |
| | | | 3.39 (dt, J = 3.2, 8.8 Hz, 1H), 3.23 (br s, 3H), 2.59 (s, |
| | | | 3H), 2.37 (td, J = 8.4, 12.0 Hz, 1H), 2.30-2.21 (m, |
| | | | 1H), 1.57 (br s, 1H), 1.45 (s, 3H), 1.37-0.93 (m, 3H) |
| 165 | Similar to | m/z 434.2 | ¹H NMR (400 MHz, CHLOROFORM-d) δ = 8.25 (br t, |
| | Example | [M + H] | J = 5.6 Hz, 1H), 7.68 (s, 1H), 7.40-7.33 (m, 2H), 7.33- |
| | 18 | | 7.28 (m, 2H), 7.27-7.23 (m, 1H), 7.01 (s, 1H), 3.71 |
| | | | (br d, J = 6.0 Hz, 2H), 3.59 (br d, J = 8.8 Hz, 1H), 3.54- |
| | | | 3.45 (m, 2H), 3.44-3.36 (m, 1H), 2.60 (s, 3H), 2.43- |
| | | | 2.32 (m, 1H), 2.31-2.21 (m, 1H), 1.45 (s, 3H), 1.39 (br |
| | | | d, J = 2.0 Hz, 2H), 1.18 (br s, 2H) |
| 166 | Similar to | m/z 435.1 | ¹H NMR (400 MHz, CHLOROFORM-d) δ = 7.87 (br d, |
| | Example | [M + H] | J = 8.0 Hz, 1H), 7.71-7.66 (m, 1H), 7.62 (br d, J = 7.6 |
| | 18 | | Hz, 1H), 7.40-7.34 (m, 2H), 7.34-7.30 (m, 2H), 7.28 |
| | | | (br s, 1H), 7.26-7.24 (m, 1H), 7.00 (s, 1H), 4.68-4.53 |
| | | | (m, 1H), 3.97-3.88 (m, 1H), 3.61 (br d, J = 8.8 Hz, |
| | | | 1H), 3.57-3.38 (m, 7H), 3.27 (br dd, J = 5.2, 12.4 Hz, |
| | | | 1H), 3.21-3.12 (m, 1H), 3.10-2.99 (m, 1H), 2.95 (dd, |
| | | | J = 7.6, 11.6 Hz, 1H), 2.64 (d, J = 7.2 Hz, 3H), 2.39 (td, |
| | | | J = 8.4, 12.0 Hz, 1H), 2.31-2.22 (m, 1H), 1.47 (s, 3H) |
| 167 | Similar to | m/z 420.2 | ¹H NMR (400 MHz, CHLOROFORM-d) δ = 7.66 (br s, |
| | Example | [M + H] | 1H), 7.61 (br d, J = 7.2 Hz, 1H), 7.41-7.34 (m, 2H), |
| | 18 | | 7.34-7.29 (m, 2H), 7.27-7.22 (m, 1H), 7.03 (br s, |
| | | | 1H), 4.33-4.18 (m, 1H), 3.61 (br d, J = 8.8 Hz, 1H), |
| | | | 3.57-3.46 (m, 2H), 3.45-3.37 (m, 1H), 2.73-2.56 (m, |
| | | | 5H), 2.39 (td, J = 8.4, 12.0 Hz, 1H), 2.33-2.23 (m, |
| | | | 3H), 1.47 (s, 3H), 1.43 (s, 3H) |
| 168 | Similar to | m/z 420.2 | ¹H NMR (400 MHz, CHLOROFORM-d) δ = 8.56 (br s, |
| | Example | [M + H] | 1H), 7.69-7.57 (m, 1H), 7.41-7.29 (m, 4H), 7.27- |
| | 18 | | 7.22 (m, 1H), 6.94 (s, 1H), 4.40 (br s, 1H), 3.60 (br dd, |
| | | | J = 3.2, 8.8 Hz, 1H), 3.55-3.45 (m, 2H), 3.44-3.25 |
| | | | (m, 3H), 3.09 (br s, 2H), 2.67-2.56 (m, 3H), 2.42- |
| | | | 2.32 (m, 1H), 2.30-2.18 (m, 1H), 2.08-1.90 (m, 2H), |
| | | | 1.76-1.63 (m, 6H), 1.46 (s, 3H) |
| 169 | Similar to | m/z 421.9 | ¹H NMR (400 MHz, DMSO-d₆) δ = 8.62 (br s, 1H), |
| | Example | [M + H] | 7.91 (s, 1H), 7.42-7.32 (m, 4H), 7.30 (s, 1H), 7.26- |
| | 18 | | 7.20 (m, 1H), 3.59 (br d, J = 9.6 Hz, 1H), 3.52 (s, 1H), |
| | | | 3.49-3.46 (m, 1H), 3.37 (br dd, J = 3.2, 5.6 Hz, 1H), |
| | | | 2.54 (s, 3H), 2.30-2.20 (m, 2H), 1.55 (s, 6H), 1.35 (s, |
| | | | 3H) |
| 170 | Similar to | m/z 350.3 | ¹H NMR (400 MHz, CHLOROFORM-d) δ = 7.67 (s, |
| | Example | [M + H]⁺ | 1H), 7.40-7.35 (m, 2H), 7.34-7.29 (m, 2H), 7.28- |
| | 18 | | 7.25 (m, 1H), 7.00 (s, 1H), 3.60 (br d, J = 8.8 Hz, 1H), |
| | | | 3.56-3.45 (m, 2H), 3.44-3.36 (m, 1H), 3.07 (br d, J = |
| | | | 4.8 Hz, 3H), 2.62 (s, 3H), 2.45-2.32 (m, 1H), 2.27 (dt, |
| | | | J = 3.6, 7.6 Hz, 1H), 1.47 (s, 3H) |
| 171 | Similar to | m/z 477.4 | ¹H NMR (400 MHz, CHLOROFORM-d) δ = 8.32 (br s, |
| | Example | [M + H]⁺ | 1H), 7.63 (s, 1H), 7.42-7.29 (m, 4H), 7.28-7.24 (m, |
| | 18 | | 1H), 7.17 (s, 1H), 7.00 (s, 1H), 5.10-4.84 (m, 1H), |
| | | | 3.55-3.46 (m, 2H), 3.44-3.36 (m, 1H), 3.73-3.36 (m, |
| | | | 3H), 3.34-2.85 (m, 5H), 2.61 (s, 4H), 2.48-2.32 (m, |
| | | | 2H), 2.31-2.13 (m, 2H), 2.12-1.96 (m, 1H), 1.67 (br |
| | | | dd, J = 5.2, 12.8 Hz, 1H), 1.46 (s, 3H) |
| 172 | Similar to | m/z 462.2 | ¹H NMR (400 MHz, DMSO-d6) δ = 9.29 (s, 1H), 7.90 |
| | Example | [M + H]⁺ | (s, 1H), 7.43-7.31 (m, 5H), 7.28-7.20 (m, 1H), 3.89 |
| | 18 | | (s, 2H), 3.62-3.59 (m, 1H), 3.53 (br d, J = 9.6 Hz, 3H), |
| | | | 2.56 (s, 3H), 2.54 (br d, J = 2.8 Hz, 2H), 2.49-2.49 (m, |
| | | | 2H), 2.26 (br t, J = 7.2 Hz, 2H), 2.08 (dd, J = 1.2, 4.4 |
| | | | Hz, 2H), 1.36 (s, 3H) |
| 173 | Similar to | m/z 448.2 | ¹H NMR (400 MHz, CHLOROFORM-d) δ = 7.89 (br s, |
| | Example | [M + H]⁺ | 1H), 7.67 (br s, 1H), 7.43-7.27 (m, 5H), 7.03 (br s, |
| | 18 | | 1H), 4.05 (br s, 2H), 3.92 (s, 2H), 3.60 (br d, J = 8.8 |
| | | | Hz, 1H), 3.56-3.46 (m, 2H), 3.45-3.36 (m, 1H), 2.63 |
| | | | (s, 3H), 2.43-2.34 (m, 1H), 2.27-2.14 (m, 5H), 1.46 |
| | | | (s, 3H) |
| 174 | Similar to | m/z 406.1 | ¹H NMR (400 MHz, CHLOROFORM-d) δ = 7.63 (br d, |
| | Example | [M + H]⁺ | J = 7.6 Hz, 1H), 7.41-7.34 (m, 2H), 7.33-7.28 (m, |
| | 18 | | 2H), 7.28-7.23 (m, 1H), 6.98 (br d, J = 6.0 Hz, 1H), |
| | | | 4.58 (br s, 1H), 4.29-4.01 (m, 2H), 3.96-3.77 (m, |
| | | | 2H), 3.58 (br t, J = 8.4 Hz, 1H), 3.55-3.44 (m, 2H), |
| | | | 3.40 (tt, J = 4.0, 8.8 Hz, 1H), 2.62 (br d, J = 6.8 Hz, |
| | | | 3H), 2.42-2.33 (m, 1H), 2.30-2.22 (m, 1H), 2.15- |
| | | | 2.06 (m, 2H), 1.46 (s, 3H) |
| 175 | Similar to | m/z 474.1 | ¹H NMR (400 MHz, CHLOROFORM-d) δ = 7.62 (br d, |
| | Example | [M + H]⁺ | J = 16.0 Hz, 2H), 7.35-7.29 (m, 2H), 7.29-7.24 (m, |
| | 18 | | 2H), 7.23-7.17 (m, 1H), 6.95 (s, 1H), 3.66 (s, 3H), |
| | | | 3.55 (br d, J = 8.8 Hz, 1H), 3.51-3.40 (m, 2H), 3.35 |
| | | | (dt, J = 3.2, 8.8 Hz, 1H), 2.57 (s, 3H), 2.38 (br s, 2H), |
| | | | 2.35-2.28 (m, 1H), 2.25-2.17 (m, 1H), 2.09-2.03 (m, |
| | | | 2H), 1.93 (br d, J = 5.6 Hz, 1H), 1.90-1.82 (m, 3H), |
| | | | 1.41 (s, 3H) |
| 176 | Similar to | m/z 450.2 | ¹H NMR (DMSO-d₆, 400 MHz): δ = 7.93 (br s, 1.0H), |
| | Example | [M + H]⁺ | 7.27-7.43 (m, 5.0H), 7.24 (br d, J = 6.4 Hz, 1.1H), 4.39- |
| | 18 | | 4.49 (m, 0.4H), 4.07-4.23 (m, 1.0H), 3.83-4.05 (m, |
| | | | 2.0H), 3.70-3.82 (m, 0.8H), 3.57-3.63 (m, 1.2H), 3.39- |
| | | | 3.54 (m, 5.1H), 3.07-3.14 (m, 1.1H), 2.52 (br s, 3.1H), |
| | | | 2.25 (br s, 2.1H), 1.35 ppm (s, 3.0H) |
| 177 | Similar to | m/z 447.4 | ¹H NMR (400 MHz, CHLOROFORM-d) δ = 7.67 (s, |
| | Example | [M + H]⁺ | 1H), 7.41-7.35 (m, 2H), 7.34-7.30 (m, 2H), 7.27- |
| | 18 | | 7.24 (m, 1H), 7.01 (s, 1H), 4.18-4.00 (m, 3H), 3.86- |
| | | | 3.80 (m, 1H), 3.72 (td, J = 5.2, 13.6 Hz, 3H), 3.63 (br |
| | | | t, J = 8.4 Hz, 3H), 3.57-3.47 (m, 3H), 3.42 (dt, J = 3.6, |
| | | | 8.8 Hz, 1H), 2.70-2.56 (m, 3H), 2.39 (td, J = 8.4, 12.4 |
| | | | Hz, 1H), 2.32-2.22 (m, 1H), 1.53-1.42 (m, 3H) |
| 178 | Similar to | m/z 462.2 | ¹H NMR (400 MHz, CHLOROFORM-d) δ = 7.68 (br s, |
| | Example | [M + H]⁺ | 1H), 7.41-7.34 (m, 2H), 7.34-7.30 (m, 2H), 7.27- |
| | 18 | | 7.24 (m, 1H), 7.04 (br s, 1H), 4.92-4.64 (m, 1H), 4.48- |
| | | | 4.26 (m, 1H), 3.78-3.32 (m, 5H), 3.21-3.05 (m, 1H), |
| | | | 2.63 (s, 3H), 2.53-2.32 (m, 2H), 2.31-2.22 (m, 1H), |
| | | | 1.84-1.63 (m, 2H), 1.47 (s, 4H), 1.25 (br s, 3H) |
| 179 | Similar to | m/z 463.3 | ¹H NMR (400 MHz, DMSO-d₆) δ = 8.77-8.49 (m, |
| | Example | [M + H]⁺ | 1H), 8.36 (br s, 1H), 7.88 (br s, 1H), 7.41-7.30 (m, |
| | 18 | | 5H), 7.23 (br t, J = 7.2 Hz, 1H), 4.61 (br s, 1H), 3.60 (br |
| | | | d, J = 9.6 Hz, 2H), 3.52 (br d, J = 9.2 Hz, 4H), 2.55 (s, |
| | | | 3H), 2.26 (br t, J = 6.8 Hz, 3H), 2.12-2.04 (m, 1H), |
| | | | 1.43-1.32 (m, 6H) |
| 180 | Similar to | m/z 464.1 | ¹H NMR (400 MHz, DMSO-d6) δ = 7.91 (s, 1H), 7.43- |
| | Example | [M + H]+ | 7.29 (m, 5H), 7.26-7.20 (m, 1H), 5.14-4.98 (m, 1H), |
| | 18 | | 4.32-4.17 (m, 1H), 4.16-3.96 (m, 2H), 3.75 (br dd, J = |
| | | | 6.0, 8.6 Hz, 1H), 3.63-3.56 (m, 2H), 3.50 (br d, J = |
| | | | 5.6 Hz, 4H), 2.92 (br dd, J = 5.6, 12.0 Hz, 1H), 2.55 (d, |
| | | | J = 10.0 Hz, 3H), 2.26 (br t, J = 6.4 Hz, 2H), 1.36 (s, |
| | | | 3H), 1.20 (s, 2H), 1.14 (s, 1H) |
| 181 | Similar to | m/z 476.2 | ¹H NMR (400 MHz, CHLOROFORM-d) δ = 7.74 (br s, |
| | Example | [M + H]+ | 1H), 7.65 (s, 1H), 7.42-7.34 (m, 2H), 7.33-7.29 (m, |
| | 18 | | 2H), 7.27-7.21 (m, 1H), 7.01 (br s, 1H), 4.23 (br s, |
| | | | 2H), 3.60 (br d, J = 8.8 Hz, 1H), 3.54-3.47 (m, 2H), |
| | | | 3.44-3.38 (m, 1H), 2.68-2.50 (m, 4H), 2.38 (br dd, J = |
| | | | 8.8, 19.8 Hz, 4H), 2.30-2.19 (m, 2H), 2.15 (br d, J = |
| | | | 13.6 Hz, 1H), 1.46 (s, 3H) |
| 182 | Similar to | m/z 478.2 | ¹H NMR (400 MHz, DMSO-d₆) δ = 7.91 (s, 1H), 7.42- |
| | Example | [M + H] | 7.31 (m, 4H), 7.29 (s, 1H), 7.26-7.20 (m, 1H), 4.04- |
| | 18 | | 3.91 (m, 3H), 3.75 (br dd, J = 6.0, 8.4 Hz, 1H), 3.70- |
| | | | 3.65 (m, 1H), 3.61-3.58 (m, 1H), 3.54-3.48 (m, 3H), |
| | | | 3.36 (br d, J = 2.8 Hz, 2H), 2.52 (br s, 3H), 2.26 (br t, J = |
| | | | 6.8 Hz, 2H), 1.90 (br d, J = 2.4 Hz, 1H), 1.86-1.77 |
| | | | (m, 1H), 1.56-1.41 (m, 2H), 1.35 (s, 3H) |
| 183 | Similar to | m/z 418.1 | ¹H NMR (400 MHz, CHLOROFORM-d) δ = 7.75 (s, |
| | Example | [M + H]⁺ | 1H), 7.60 (s, 1H), 7.35-7.29 (m, 2H), 7.28-7.24 (m, |
| | 18 | | 2H), 7.22-7.18 (m, 1H), 6.96 (s, 1H), 4.49 (s, 1H), |
| | | | 3.88 (s, 2H), 3.55 (d, J = 8.8 Hz, 1H), 3.52-3.41 (m, |
| | | | 2H), 3.36 (dt, J = 3.6, 8.7 Hz, 1H), 2.57 (s, 3H), 2.33 |
| | | | (td, J = 8.3, 12.1 Hz, 1H), 2.28-2.17 (m, 3H), 2.08- |
| | | | 1.99 (m, 2H), 1.41 (s, 3H) |
| 184 | Similar to | m/z 337.3 | ¹H NMR (400 MHz, CHLOROFORM-d) δ = 8.05 (s, |
| | Example | [M + H]+ | 1H), 7.40-7.31 (m, 4H), 7.27-7.23 (m, 1H), 7.06 (s, |
| | 18 | | 1H), 3.66 (br d, J = 8.8 Hz, 1H), 3.61-3.51 (m, 2H), |
| | | | 3.46 (dt, J = 3.2, 8.6 Hz, 1H), 2.73 (s, 3H), 2.44-2.35 |
| | | | (m, 1H), 2.31-2.23 (m, 1H), 1.47 (s, 3H) |
| 185 | Similar to | m/z 417.0 | ¹H NMR (400 MHz, DMSO-d₆): δ 8.90 (s, 1H), 8.27 (s, |
| | Example | [M + H]⁺ | 1H), 7.89 (d, J = 2.0 Hz, 1H), 7.43-7.29 (m, 5H), 7.27- |
| | 18 | | 7.20 (m, 1H), 3.63-3.56 (m, 1H), 3.55-3.45 (m, 2H), |
| | | | 3.42-3.32 (m, 1H), 3.19 (br d, J = 10.4 Hz, 1H), 3.13 |
| | | | (br dd, J = 3.2, 11.2 Hz, 1H), 3.00-2.85 (m, 2H), 2.54 |
| | | | (s, 3H), 2.26 (br t, J = 7.2 Hz, 2H), 1.75-1.66 (m, 1H), |
| | | | 1.35 (s, 3H), 1.01 (t, J = 5.2 Hz, 1H), 0.98-0.93 (m, 1H). |
| 186 | Similar to | m/z 431.1 | ¹H NMR (400 MHz, DMSO-d₆): δ 8.65 (br d, J = 8.4 |
| | Example | [M + H]⁺ | Hz, 1H), 8.36 (s, 1H), 7.89 (d, J = 1.6 Hz, 1H), 7.41- |
| | 18 | | 7.33 (m, 5H), 7.28-7.18 (m, 1H), 4.31-4.26 (m, 1H), |
| | | | 3.62-3.58 (m, 1H), 3.52 (d, J = 9.6 Hz, 1H), 3.50-3.41 |
| | | | (m, 2H), 3.40-3.34 (m, 1H), 3.26 (br d, J = 11.2 Hz, |
| | | | 1H), 3.21 (dd, J = 3.6, 12.0 Hz, 1H), 2.89 (d, J = 11.2 |
| | | | Hz, 1H), 2.56 (s, 3H), 2.26 (br t, J = 7.2 Hz, 2H), 1.36 (s, |
| | | | 3H), 0.80-0.71 (m, 2H), 0.70-0.58 (m, 2H). |
| 187 | Similar to | m/z 431.1 | ¹H NMR (400 MHz, DMSO-d₆) δ = 8.27 (d, J = 8.8 Hz, |
| | Example | [M + H]⁺ | 1H), 7.93-7.88 (m, 1H), 7.42-7.31 (m, 5H), 7.27-7.20 |
| | 18 | | (m, 1H), 4.17-4.07 (m, 1H), 3.63-3.58 (m, 1H), 3.54- |
| | | | 3.47 (m, 2H), 3.41-3.35 (m, 2H), 3.14 (dd, J = 6.0, 11.6 |
| | | | Hz, 1H), 2.97-2.85 (m, 2H), 2.61 (d, J = 10.8 Hz, 1H), |
| | | | 2.56 (s, 3H), 2.26 (t, J = 7.2 Hz, 2H), 1.36 (s, 3H), 0.74 |
| | | | (ddd, J = 3.2, 5.6, 9.2 Hz, 1H), 0.69-0.62 (m, 1H), 0.57- |
| | | | 0.54 (m, 1H), 0.52-0.45 (m, 1H). |
| 188 | Similar to | m/z 475.3 | ¹H NMR (400 MHz, DMSO-d₆) δ = 8.39 (d, J = 8.4 Hz, |
| | Example | [M + H]⁺ | 1H), 7.90-7.85 (m, 1H), 7.39 (d, J = 1.2 Hz, 2H), 7.34 |
| | 18 | | (s, 2H), 7.33-7.31 (m, 1H), 7.26-7.21 (m, 1H), 4.46- |
| | | | 4.35 (m, 1H), 4.10-3.99 (m, 1H), 3.92-3.83 (m, 1H), |
| | | | 3.62-3.57 (m, 1H), 3.55-3.46 (m, 2H), 3.41-3.34 (m, |
| | | | 1H), 3.09 (t, J = 11.6 Hz, 1H), 2.69-2.63 (m, 1H), 2.55 |
| | | | (s, 3H), 2.33 (d, J = 7.2 Hz, 2H), 2.26 (t, J = 7.2 Hz, 2H), |
| | | | 1.94-1.94 (m, 1H), 1.86-1.74 (m, 2H), 1.61-1.45 (m, |
| | | | 2H), 1.36 (s, 3H), 0.99 (t, J = 7.2 Hz, 3H). |
| 189 | Similar to | m/z 419.1 | ¹H NMR (400 MHz, DMSO-d₆) δ ppm 8.30-8.39 (m, |
| | Example | [M + H]⁺ | 2 H), 7.90 (d, J = 1.6 Hz, 1 H), 7.32-7.43 (m, 5 H), 7.21- |
| | 18 | | 7.27 (m, 1 H), 3.60 (br d, J = 10.0 Hz, 2 H), 3.46-3.55 |
| | | | (m, 2 H), 3.33-3.42 (m, 1 H), 3.18 (br t, J = 7.2 Hz, 2 |
| | | | H), 3.05 (br d, J = 11.6 Hz, 1 H), 2.56 (s, 3 H), 2.42- |
| | | | 2.50 (m, 1 H), 2.27 (t, J = 7.2 Hz, 2 H), 1.83-1.99 (m, 1 |
| | | | H), 1.52 (s, 3 H), 1.36 (s, 3 H) |
| 190 | Similar to | m/z 483.2 | ¹H NMR (400 MHz, DMSO-d6) δ 8.76-8.86 (m, 1 H), |
| | Example | [M + H]⁺ | 8.47 (s, 1 H), 8.14 (s, 2 H), 7.90 (d, J = 1.6 Hz, 1 H), |
| | 18 | | 7.32-7.43 (m, 5 H), 7.20-7.28 (m, 1 H), 4.63-4.76 |
| | | | (m, 1 H), 3.58-3.69 (m, 2 H), 3.46-3.56 (m, 3 H), |
| | | | 3.35-3.41 (m, 3 H), 2.56 (s, 3 H), 2.24-2.31 (m, 3 H), |
| | | | 2.13-2.23 (m, 1 H), 1.36 (s, 3 H). |
| 191 | Similar to | m/z 421.1 | ¹H NMR (400 MHz, DMSO-d6) δ = 8.38 (s, 1H), 8.10 |
| | Example | [M + H]⁺ | (d, J = 7.6 Hz, 1H), 7.92 (d, J = 1.6 Hz, 1H), 7.42-7.28 |
| | 18 | | (m, 5H), 7.27-7.19 (m, 1H), 4.46-4.36 (m, 1H), 4.30 |
| | | | (br d, J = 3.6 Hz, 1H), 3.59 (br d, J = 9.6 Hz, 1H), 3.53- |
| | | | 3.46 (m, 2H), 3.46-3.38 (m, 1H), 3.38-3.31 (m, 2H), |
| | | | 3.09 (br d, J = 12.0 Hz, 1H), 3.02 (br t, J = 10.4 Hz, |
| | | | 1H), 2.54 (s, 2H), 2.25 (br t, J = 6.9 Hz, 2H), 1.34 (s, |
| | | | 3H). |
| 192 | Similar to | m/z 463.1 | ¹H NMR (400 MHz, DMSO-d₆) δ = 8.87 (d, J = 7.6 Hz, |
| | Example | [M + H]⁺ | 1H), 7.89 (d, J = 1.6 Hz, 1H), 7.43-7.30 (m, 5H), 7.28- |
| | 18 | | 7.19 (m, 1H), 4.75-4.58 (m, 1H), 4.02 (s, 2H), 3.63- |
| | | | 3.57 (m, 2H), 3.56-3.46 (m, 4H), 3.41-3.33 (m, 3H), |
| | | | 3.31-3.21 (m, 2H), 2.57 (s, 3H), 2.43-2.30 (m, 1H), |
| | | | 2.26 (br t, J = 7.2 Hz, 2H), 2.13-1.97 (m, 1H), 1.36 (s, |
| | | | 3H). |
| 193 | Similar to | m/z 469.2 | ¹H NMR (400 MHz, CHLOROFORM-d) δ = 7.58 (d, J = |
| | Example | [M + H]⁺ | 1.2 Hz, 1H), 7.52 (br d, J = 8.4 Hz, 1H), 7.33-7.15 |
| | 18 | | (m, 5H), 6.92 (s, 1H), 6.01-5.66 (m, 1H), 4.74-4.60 |
| | | | (m, 1H), 3.53 (d, J = 8.8 Hz, 1H), 3.49-3.38 (m, 2H), |
| | | | 3.37-3.28 (m, 1H), 3.00 (dt, J = 4.4, 8.8 Hz, 1H), 2.89- |
| | | | 2.74 (m, 4H), 2.55 (s, 3H), 2.43 (q, J = 8.4 Hz, 1H), |
| | | | 2.38-2.26 (m, 2H), 2.20 (br dd, J = 3.6, 7.2 Hz, 1H), |
| | | | 1.85-1.70 (m, 1H), 1.39 (s, 3H) |
| 194 | Similar to | m/z 552.2 | ¹H NMR (400 MHz, CHLOROFORM-d) δ = 7.72- |
| | Example | [M + H]⁺ | 7.61 (m, 2H), 7.54-7.48 (m, 1H), 7.41-7.29 (m, 4H), |
| | 18 | | 7.26-7.22 (m, 1H), 7.01 (s, 1H), 6.64 (d, J = 8.4 Hz, |
| | | | 1H), 4.39 (br dd, J = 7.2, 12.0 Hz, 1H), 4.14-4.01 (m, |
| | | | 2H), 3.88-3.76 (m, 3H), 3.63-3.47 (m, 5H), 3.41 (dt, |
| | | | J = 3.2, 8.4 Hz, 1H), 3.25-3.10 (m, 2H), 2.65 (s, 3H), |
| | | | 2.43-2.34 (m, 1H), 2.31-2.23 (m, 1H), 1.47 (s, 3H) |
| 195 | Similar to | m/z 566.3 | ¹H NMR (400 MHz, CHLOROFORM-d) δ = 7.73- |
| | Example | [M + H]⁺ | 7.59 (m, 2H), 7.50 (d, J = 7.2 Hz, 1H), 7.40-7.30 (m, |
| | 18 | | 4H), 7.27-7.24 (m, 1H), 7.04-6.94 (m, 2H), 4.73- |
| | | | 4.58 (m, 2H), 4.52 (s, 1H), 4.25-4.18 (m, 1H), 3.94 (d, |
| | | | J = 12.4 Hz, 1H), 3.62 (d, J = 8.8 Hz, 1H), 3.55 (br d, J = |
| | | | 8.0 Hz, 1H), 3.50 (d, J = 8.8 Hz, 1H), 3.43 (br d, J = |
| | | | 3.6 Hz, 1H), 3.05-2.94 (m, 1H), 2.90-2.79 (m, 1H), |
| | | | 2.66 (s, 3H), 2.38 (s, 1H), 2.28 (br s, 1H), 2.19 (br d, J = |
| | | | 6.4 Hz, 1H), 2.13 (br d, J = 7.2 Hz, 1H), 1.92 (br d, J = |
| | | | 13.2 Hz, 2H), 1.67 (br d, J = 12.8 Hz, 1H), 1.48 (s, |
| | | | 3H) |
| 196 | Similar to | m/z 406.2 | |
| | Example | [M + H]⁺ | |
| | 18 | | |
| 197 | Similar to | m/z 448.3 | |
| | Example | [M + H]⁺ | |
| | 18 | | |
| 198 | Similar to | m/z 420.4 | |
| | Example | [M + H]⁺ | |
| | 18 | | |
| 199 | Similar to | m/z 445.4 | |
| | Example | [M + H]⁺ | |
| | 18 | | |
| 200 | Similar to | m/z 405.4 | |
| | Example | [M + H]⁺ | |
| | 18 | | |
| 201 | Similar to | m/z 431.1 | |
| | Example | [M + H]⁺ | |
| | 18 | | |
| 202 | Similar to | m/z 447.9 | |
| | Example | [M + H]⁺ | |
| | 18 | | |
| 203 | Similar to | m/z 473.2 | |
| | Example | [M + H]⁺ | |
| | 18 | | |
| 204 | Similar to | m/z 446.1 | |
| | Example | [M + H]⁺ | |
| | 18 | | |
| 205 | Similar to | m/z 474.2 | |
| | Example | [M + H]⁺ | |
| | 18 | | |
| 206 | Similar to | m/z 422.8 | |
| | Example | [M + H]⁺ | |
| | 18 | | |
| 207 | Similar to | m/z 460.1 | |
| | Example | [M + H]⁺ | |
| | 18 | | |
| 208 | Similar to | m/z 474.2 | |
| | Example | [M + H]⁺ | |
| | 18 | | |
| 209 | Similar to | m/z 476 | |
| | Example | [M + H]⁺ | |
| | 18 | | |
| 210 | Similar to | m/z 446.2 | |
| | Example | [M + H]⁺ | |
| | 18 | | |
| 211 | Similar to | m/z 446.2 | |
| | Example | [M + H]⁺ | |
| | 18 | | |
| 212 | Similar to | m/z 461.2 | |
| | Example | [M + H]⁺ | |
| | 18 | | |
| 439 | Similar to | m/z 446.1 | |
| | Example | [M + H]⁺ | |
| | 18 | | |

### Example 19

### 6-(3-(8-methyl-6-((R)-3-methyl-3-phenylpyrrolidin-1-yl)-[1,2,4]triazolo[1,5-a]pyridine-2-carboxamido)pyrrolidin-1-yl)picolinic acid (Compound 19)

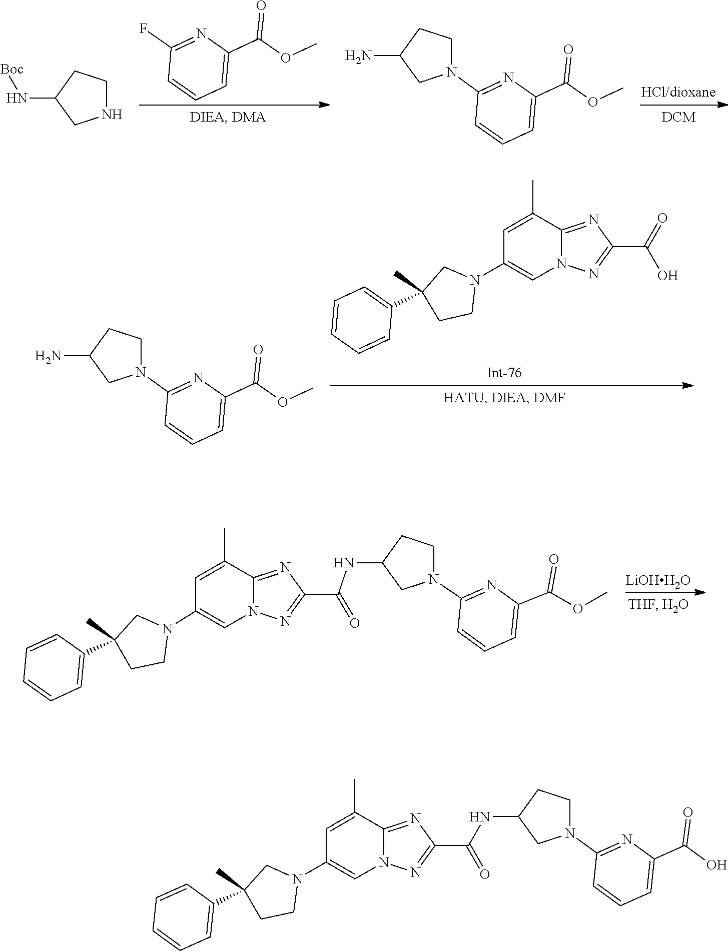

### Step 1: methyl 6-(3-aminopyrrolidin-1-yl)picolinate

To a solution of tert-butyl N-pyrrolidin-3-ylcarbamate (300 mg, 1.61 mmol, 1 eq) and methyl 6-fluoropyridine-2-carboxylate (249.87 mg, 1.61 mmol, 1 eq) in DMA (3 mL) was added DIEA (624.53 mg, 4.83 mmol, 841.68 μL, 3 eq). The mixture was stirred at 100° C. for 2 h. LCMS showed starting material was consumed and the desired mass was detected. The reaction mixture was added H₂O (5 mL) and then extracted with EtOAc (10 mL×3). The combined organic phase was washed with brine (10 mL×3), dried over anhydrous Na2SO4, filtered and concentrated in vacuo. The residue was purified by flash silica gel chromatography (ISCO®; 12 g SepaFlash® Silica Flash Column, Eluent of 50~60% Ethyl acetate/Petroleum ethergradient @80 mL/min). to give compound methyl 6-[3-(tert-butoxycarbonylamino)pyrrolidin-1-yl]pyridine-2-carboxylate (250 mg, 48.30% yield) as a brown oil.

LC-MS [ESI, M+1]: 322.5

### Step 2: methyl 6-(3-aminopyrrolidin-1-yl)picolinate

To a solution of methyl 6-(3-aminopyrrolidin-1-yl)pyridine-2-carboxylate (250 mg, 1.13 mmol, 1 eq) in DCM (1 mL) was added HCl/dioxane (2 M, 564.96 μL, 1 eq). The mixture was stirred at 25° C. for 1 h. LCMS showed starting material was consumed and the desired mass was detected. The reaction was concentrated under reduced pressure to give compound methyl 6-(3-aminopyrrolidin-1-yl)pyridine-2-carboxylate (300 mg, crude, HCl) as a yellow oil. LC-MS [ESI, M+1]: 222.5

### Step 3: methyl 6-(3-(8-methyl-6-((R)-3-methyl-3-phenylpyrrolidin-1-yl)-[1,2,4]triazolo[1,5-a]pyridine-2-carboxamido)pyrrolidin-1-yl)picolinate

To a solution of methyl 6-(3-aminopyrrolidin-1-yl)pyridine-2-carboxylate (114.92 mg, 445.92 mol, 1.5 eq, HCl) and 8-methyl-6-[(3R)-3-methyl-3-phenyl-pyrrolidin-1-yl]-[1,2,4]triazolo[1,5-a]pyridine-2-carboxylic acid (100 mg, 297.28 mol, 1 eq) in DMF (1 mL) was added HATU (169.55 mg, 445.92 mol, 1.5 eq) and DIEA (115.26 mg, 891.83 mol, 155.34 μL, 3 eq). The mixture was stirred at 25° C. for 1 h. LCMS showed starting material was consumed and the desired mass was detected. The reaction mixture was added H₂O (5 mL) and then extracted with EtOAc (10 mL×3). The combined organic phase was washed with brine (5 mL×3), dried over anhydrous Na2SO4, filtered and concentrated in vacuo. The residue was purified by prep-TLC (SiO₂, DCM:MeOH=10:1) to give compound methyl 6-[3-[[8-methyl-6-[(3R)-3-methyl-3-phenyl-pyrrolidin-1-yl]-[1,2,4]triazolo[1,5-a]pyridine-2-carbonyl]amino]pyrrolidin-1-yl]pyridine-2-carboxylate (130 mg, 81% yield) as a yellow solid.

LC-MS [ESI, M+1]: 540.3

### Step 4: 6-(3-(8-methyl-6-((R)-3-methyl-3-phenylpyrrolidin-1-yl)-[1,2,4]triazolo[1,5-a]pyridine-2-carboxamido)pyrrolidin-1-yl)picolinic acid

To a solution of methyl 6-[3-[[8-methyl-6-[(3R)-3-methyl-3-phenyl-pyrrolidin-1-yl]-[1,2,4]triazolo[1,5-a]pyridine-2-carbonyl]amino]pyrrolidin-1-yl]pyridine-2-carboxylate (130 mg, 240.91 mol, 1 eq) in THF (1 mL) and H₂O (0.5 mL) was added LiOH·H₂O (20.22 mg, 481.81 mol, 2 eq). The mixture was stirred at 25° C. for 0.5 h. LCMS showed starting material was consumed and the desired mass was detected. The reaction mixture was added HCOOH until pH around 4 and then concentrated in vacuo. The residue was purified by prep-HPLC (column: Phenomenex luna C18 150×25 mm×10 um; mobile phase: [water(FA)-ACN]; gradient:30%-60% B over 10 min). to give compound 6-[3-[[8-methyl-6-[(3R)-3-methyl-3-phenyl-pyrrolidin-1-yl]-[1,2,4]triazolo[1,5-a]pyridine-2-carbonyl]amino]pyrrolidin-1-yl]pyridine-2-carboxylic acid (87.97 mg, 68% yield) as a white solid.

LC-MS [ESI, M+1]: 526.4.

¹H NMR (400 MHz, CHLOROFORM-d) δ=7.71-7.61 (m, 2H), 7.55 (br s, 1H), 7.50 (br d, J=7.2 Hz, 1H), 7.41-7.34 (m, 2H), 7.33-7.29 (m, 2H), 7.25 (br s, 1H), 7.00 (s, 1H), 6.66 (br d, J=8.4 Hz, 1H), 4.94 (sxt, J=5.6 Hz, 1H), 3.94 (br dd, J=6.4, 10.4 Hz, 1H), 3.79-3.71 (m, 1H), 3.70-3.64 (m, 1H), 3.60 (br d, J=9.2 Hz, 2H), 3.55-3.45 (m, 2H), 3.41 (dt, J=3.6, 8.8 Hz, 1H), 2.61 (s, 3H), 2.49 (br dd, J=6.8, 13.2 Hz, 1H), 2.42-2.34 (m, 1H), 2.30-2.22 (m, 2H), 1.47 (s, 3H).

| Cmpd. No. | Synthesis | LC/MS | ¹H NMR |
|---|---|---|---|
| 213 | Similar to | m/z 552.2 | ¹H NMR (400 MHz, CHLOROFORM-d) δ = 7.70- |
| | Example 19 | [M + H]⁺ | 7.59 (m, 2H), 7.49 (d, J = 7.1 Hz, 1H), 7.40-7.34 |
| | | | (m, 2H), 7.33-7.29 (m, 2H), 7.27-7.22 (m, 1H), |
| | | | 7.00 (s, 1H), 6.63 (d, J = 8.5 Hz, 1H), 4.38 (br dd, |
| | | | J = 7.0, 12.0 Hz, 1H), 4.14-3.99 (m, 2H), 3.87-3.75 |
| | | | (m, 3H), 3.60 (br d, J = 8.9 Hz, 1H), 3.57-3.45 (m, |
| | | | 4H), 3.41 (dt, J = 3.4, 8.6 Hz, 1H), 3.18 (br d, |
| | | | J = 4.3 Hz, 2H), 2.63 (s, 3H), 2.43-2.32 (m, 1H), |
| | | | 2.31-2.21 (m, 1H), 1.46 (s, 3H) |
| 214 | Similar to | m/z 538.2 | ¹H NMR (400 MHz, CHLOROFORM-d) δ = 7.69- |
| | Example 19 | [M + H]⁺ | 7.62 (m, 2H), 7.54-7.44 (m, 2H), 7.41-7.35 (m, |
| | | | 2H), 7.34-7.30 (m, 2H), 7.26 (br s, 1H), 7.02 (s, |
| | | | 1H), 6.64 (d, J = 8.4 Hz, 1H), 3.99 (d, J = 10.3 Hz, |
| | | | 2H), 3.61 (br d, J = 9.3 Hz, 3H), 3.55-3.47 (m, |
| | | | 2H), 3.42 (dt, J = 3.8, 8.6 Hz, 1H), 2.82 (d, J = 2.3 |
| | | | Hz, 1H), 2.63 (s, 3H), 2.42-2.35 (m, 1H), 2.30- |
| | | | 2.24 (m, 1H), 2.12 (br s, 2H), 1.47 (s, 3H) |
| 215 | Similar to | m/z 566.2 | ¹H NMR (400 MHz, CHLOROFORM-d) δ = 8.41- |
| | Example 19 | [M + H]⁺ | 8.13 (m, 2H), 8.11-7.91 (m, 1H), 7.64 (s, 1H), |
| | | | 7.42-7.34 (m, 2H), 7.34-7.29 (m, 2H), 7.27-7.21 (m, |
| | | | 1H), 7.03-6.90 (m, 1H), 5.62 (br d, J = 10.6 Hz, |
| | | | 1H), 5.33-5.22 (m, 1H), 5.15 (br d, J = 11.0 Hz, |
| | | | 1H), 4.93-4.62 (m, 3H), 4.39 (br d, J = 10.3 Hz, |
| | | | 1H), 4.20-4.06 (m, 1H), 3.60 (br d, J = 9.0 Hz, |
| | | | 1H), 3.54 (br s, 1H), 3.52-3.49 (m, 1H), 3.47-3.39 |
| | | | (m, 2H), 2.72 (br t, J = 7.3 Hz, 2H), 2.66-2.54 (m, |
| | | | 3H), 2.43-2.33 (m, 1H), 2.30-2.19 (m, 1H), 1.55- |
| | | | 1.39 (m, 3H) |
| 216 | Similar to | m/z 553.2 | ¹H NMR (400 MHz, CHLOROFORM-d) δ = 7.91- |
| | Example 19 | [M + H]⁺ | 7.72 (m, 2H), 7.63 (br s, 1H), 7.41-7.34 (m, 2H), |
| | | | 7.34-7.29 (m, 2H), 7.24 (br s, 1H), 7.14-6.92 (m, |
| | | | 2H), 5.35-5.05 (m, 2H), 4.88 (br d, J = 11.0 Hz, |
| | | | 1H), 4.76 (br d, J = 10.3 Hz, 1H), 4.66 (br d, J = |
| | | | 10.9 Hz, 1H), 4.25-4.13 (m, 1H), 3.60 (br d, J = |
| | | | 8.8 Hz, 1H), 3.54-3.46 (m, 2H), 3.41 (br d, J = 7.4 |
| | | | Hz, 1H), 2.62 (br s, 3H), 2.46 (br d, J = 8.0 Hz, 1H), |
| | | | 2.40-2.33 (m, 1H), 2.31-2.18 (m, 2H), 2.11-1.98 |
| | | | (m, 2H), 1.46 (s, 3H) |
| 217 | Similar to | m/z 512.2 | ¹H NMR (400 MHz, CHLOROFORM-d) δ = 7.87 |
| | Example 19 | [M + H]⁺ | (br d, J = 7.5 Hz, 1H), 7.74-7.63 (m, 2H), 7.56 (br |
| | | | d, J = 7.1 Hz, 1H), 7.41-7.34 (m, 2H), 7.34-7.29 |
| | | | (m, 2H), 7.25 (br s, 1H), 7.02 (s, 1H), 6.58 (br d, J = |
| | | | 8.3 Hz, 1H), 5.19 (br d, J = 5.1 Hz, 1H), 4.54 (br t, |
| | | | J = 8.1 Hz, 2H), 4.16-4.04 (m, 2H), 3.61 (br d, J = |
| | | | 8.5 Hz, 1H), 3.58-3.46 (m, 2H), 3.46-3.36 (m, |
| | | | 1H), 2.63 (s, 3H), 2.42-2.34 (m, 1H), 2.30-2.23 |
| | | | (m, 1H), 1.47 (s, 3H) |
| 218 | Similar to | m/z 572.2 | ¹H NMR (400 MHz, CHLOROFORM-d) δ = 7.65 |
| | Example 19 | [M + H]⁺ | (br d, J = 7.3 Hz, 1H), 7.47 (d, J = 4.9 Hz, 1H), |
| | | | 7.41-7.34 (m, 2H), 7.34-7.29 (m, 2H), 7.27-7.22 (m, |
| | | | 1H), 7.00 (br d, J = 3.0 Hz, 1H), 4.19 (br d, J = 6.5 |
| | | | Hz, 1H), 4.02 (br s, 1H), 3.94-3.86 (m, 1H), 3.84- |
| | | | 3.73 (m, 1H), 3.70-3.46 (m, 7H), 3.45-3.39 (m, |
| | | | 1H), 2.64 (br d, J = 6.6 Hz, 3H), 2.43-2.34 (m, |
| | | | 1H), 2.31-2.23 (m, 1H), 2.23-1.96 (m, 4H), 1.47 |
| | | | (br d, J = 5.0 Hz, 3H) |
| 219 | Similar to | m/z 540.2 | ¹H NMR (CHLOROFORM-d, 400 MHz): δ = 7.52- |
| | Example 19 | [M + H]⁺ | 7.70 (m, 2.1H), 7.46 (d, J = 7.2 Hz, 1.0H), 7.28-7.40 |
| | | | (m, 3.0H), 7.11-7.27 (m, 3.1H), 6.89-7.04 (m, 2.0H), |
| | | | 4.24 (br d, J = 10.4 Hz, 2.1H), 3.86 (br d, J = |
| | | | 13.2 Hz, 1.2H), 3.55 (br d, J = 8.8 Hz, 1.1H), 3.40- |
| | | | 3.51 (m, 2.2H), 3.35 (br d, J = 3.2 Hz, 2.2H), 3.22- |
| | | | 3.30 (m, 1.2H), 2.55 (s, 3.2H), 2.31 (s, 1.2H), 2.22 |
| | | | (br dd, J = 7.2, 3.9 Hz, 1.2H), 2.08 (br d, J = 5.6 Hz, |
| | | | 1.1H), 1.74-1.90 (m, 2.2H), 1.60-1.72 (m, 1.2H), |
| | | | 1.41 ppm (s, 3.0H) |
| 220 | Similar to | m/z 582.2 | ¹H NMR (CHLOROFORM-d, 400 MHz): δ = 7.66 |
| | Example 19 | [M + H]⁺ | (br s, 2.0H), 7.57 (br d, J = 7.2 Hz, 1.0H), 7.48 (br s, |
| | | | 1.0H), 7.35 (br dd, J = 17.2, 6.9 Hz, 5.0H), 7.02 (br s, |
| | | | 0.9H), 6.63 (br dd, J = 5.6, 1.2 Hz, 0.8H), 4.89 (br s, |
| | | | 0.9H), 4.18 (br d, J = 3.6 Hz, 1.0H), 3.83-4.07 (m, |
| | | | 2.0H), 3.58-3.76 (m, 4.0H), 3.52 (br dd, J = 20.0, 8.8 |
| | | | Hz, 2.0H), 3.44 (br d, J = 11.2 Hz, 1.0H), 2.65 (br s, |
| | | | 3.0H), 2.59 (br s, 1.2H), 2.35-2.44 (m, 2.0H), 2.21- |
| | | | 2.31 (m, 3.1H), 1.48 ppm (s, 3.0H) |
| 221 | Similar to | m/z 553.2 | ¹H NMR (CHLOROFORM-d, 400 MHz): δ = 7.86 |
| | Example 19 | [M + H]⁺ | (br s, 2.0H), 7.67 (br d, J = 6.0 Hz, 1.0H), 7.38 (br d, |
| | | | J = 7.2 Hz, 2.0H), 7.34 (br s, 2.0H), 7.18-7.28 (m, |
| | | | 1.0H), 7.03 (br s, 1.9H), 5.13-5.30 (m, 1.0H), 4.67- |
| | | | 4.92 (m, 2.0H), 4.27-4.47 (m, 2.0H), 3.59-3.70 (m, |
| | | | 1.1H), 3.47-3.59 (m, 2.0H), 3.39-3.47 (m, 1.0H), |
| | | | 2.84-2.95 (m, 2.1H), 2.66 (br d, J = 6.8 Hz, 3.1H), |
| | | | 2.45-2.55 (m, 2.1H), 2.35-2.44 (m, 1.1H), 2.29 (br |
| | | | d, J = 4.4 Hz, 1.2H), 1.48 ppm (br s, 3.0H) |
| 222 | Similar to | m/z 582.3 | 1H NMR (CHLOROFORM-d, 400 MHz): δ = 7.67- |
| | Example 19 | [M + H]+ | 7.78 (m, 0.9H), 7.47-7.67 (m, 2.0H), 7.27-7.43 (m, |
| | | | 5.0H), 6.82-7.08 (m, 2.0H), 4.03-4.29 (m, 2.3H), |
| | | | 3.26-4.00 (m, 12.0H), 2.53-2.69 (m, 3.1H), 2.34- |
| | | | 2.43 (m, 1.1H), 2.27 (dt, J = 7.6, 3.6 Hz, 2.0H), 2.08 |
| | | | (br s, 1.0H), 1.47 ppm (br d, J = 4.8 Hz, 3.0H) |
| 223 | Similar to | m/z 524.3 | 1H NMR (400 MHz, CHLOROFORM-d) δ = 11.08-10.19 |
| | Example 19 | [M + H]+ | (m, 1H), 7.67 (s, 1H), 7.49 (br d, J = 8.0 Hz, |
| | | | 1H), 7.42-7.36 (m, 2H), 7.35-7.31 (m, 2H), 7.27 |
| | | | (br d, J = 1.3 Hz, 1H), 7.18 (br d, J = 7.2 Hz, 1H), |
| | | | 7.03 (s, 1H), 5.64-5.58 (m, 1H), 5.28 (s, 1H), 4.69- |
| | | | 4.57 (m, 1H), 4.08 (s, 2H), 3.98 (s, 2H), 3.62 (d, |
| | | | J = 9.2 Hz, 1H), 3.59-3.48 (m, 2H), 3.43 (dt, J = 3.6, |
| | | | 8.8 Hz, 1H), 2.80 (br t, J = 10.0 Hz, 2H), 2.65 (s, |
| | | | 3H), 2.45-2.34 (m, 3H), 2.32-2.25 (m, 1H), 1.49 |
| | | | (s, 3H) |
| 224 | Similar to | m/z 556.2 | 1H NMR (400 MHz, CHLOROFORM-d) δ = 7.86 |
| | Example 19 | [M + H]+ | (t, J = 8.0 Hz, 1H), 7.80 (br d, J = 7.6 Hz, 1H), |
| | | | 7.73 (s, 1H), 7.42-7.34 (m, 2H), 7.34-7.30 (m, 2H), |
| | | | 7.25 (br s, 1H), 7.10 (br d, J = 8.0 Hz, 1H), 7.04 (br |
| | | | t, J = 6.0 Hz, 2H), 4.51 (br d, J = 2.8 Hz, 1H), 3.62 |
| | | | (d, J = 9.2 Hz, 1H), 3.58-3.47 (m, 2H), 3.43 (dt, |
| | | | J = 3.6, 8.7 Hz, 1H), 2.72 (br s, 1H), 2.66 (s, 3H), |
| | | | 2.43-2.36 (m, 1H), 2.30-2.25 (m, 1H), 2.07 (br s, |
| | | | 2H), 1.84 (br s, 6H), 1.48 (s, 3H) |
| 225 | Similar to | m/z 556.2 | 1H NMR (400 MHz, CHLOROFORM-d) δ = 7.97 |
| | Example 19 | [M + H]+ | (t, J = 8.0 Hz, 1H), 7.69 (s, 1H), 7.40-7.37 (m, |
| | | | 1H), 7.37-7.28 (m, 4H), 7.27 (s, 1H), 7.14-7.07 |
| | | | (m, 1H), 7.06-6.97 (m, 2H), 4.22-4.08 (m, 1H), |
| | | | 3.61 (br d, J = 8.8 Hz, 1H), 3.57-3.48 (m, 2H), |
| | | | 3.42 (dt, J = 3.6, 8.7 Hz, 1H), 2.65 (s, 3H), 2.63- |
| | | | 2.55 (m, 1H), 2.43-2.35 (m, 1H), 2.28 (br dd, J = |
| | | | 4.0, 7.4 Hz, 3H), 2.00 (br d, J = 11.2 Hz, 2H), 1.67 |
| | | | (q, J = 12.0 Hz, 2H), 1.60-1.49 (m, 2H), 1.47 (s, |
| | | | 3H) |
| 226 | Similar to | m/z 539.2 | ¹H NMR (400 MHz, DMSO-d₆) δ = 8.60 (d, J = 5.2 |
| | Example 19 | [M + H]+ | Hz, 1H), 8.31 (d, J = 8.0 Hz, 1H), 8.02 (s, 1H), 7.92 |
| | | | (s, 1H), 7.60 (dd, J = 1.6, 5.1 Hz, 1H), 7.42-7.30 |
| | | | (m, 5H), 7.26-7.20 (m, 1H), 4.25-4.13 (m, 1H), |
| | | | 3.62-3.58 (m, 1H), 3.55-3.48 (m, 3H), 2.84-2.76 |
| | | | (m, 1H), 2.56 (s, 3H), 2.26 (br t, J = 7.2 Hz, 2H), |
| | | | 1.96-1.83 (m, 4H), 1.79-1.69 (m, 4H), 1.36 (s, |
| | | | 3H) |
| 227 | Similar to | m/z 540.2 | 1H NMR (400 MHz, CHLOROFORM-d) δ = 8.31 |
| | Example 19 | [M + H]+ | (d, J = 5.2 Hz, 1H), 7.69 (d, J = 1.2 Hz, 1H), 7.60 |
| | | | (br d, J = 8.4 Hz, 1H), 7.42-7.34 (m, 2H), 7.32 (br |
| | | | d, J = 5.6 Hz, 3H), 7.25 (br s, 1H), 7.15 (d, J = 5.2 |
| | | | Hz, 1H), 7.03 (s, 1H), 4.38 (br d, J = 12.8 Hz, 3H), |
| | | | 3.61 (d, J = 8.8 Hz, 1H), 3.56-3.46 (m, 2H), 3.41 |
| | | | (dt, J = 3.6, 8.7 Hz, 1H), 3.14 (br t, J = 11.6 Hz, |
| | | | 2H), 2.63 (s, 3H), 2.41-2.37 (m, 1H), 2.30-2.26 |
| | | | (m, 1H), 2.15 (br d, J = 10.0 Hz, 2H), 1.72-1.59 |
| | | | (m, 2H), 1.47 (s, 3H) |
| 228 | Similar to | m/z 552.2 | ¹H NMR (400 MHz, CHLOROFORM-d) δ = 7.62 |
| | Example 19 | [M + H]⁺ | (s, 1H), 7.60-7.55 (m, 1H), 7.52 (br d, J = 7.6 Hz, |
| | | | 1H), 7.44 (d, J = 7.2 Hz, 1H), 7.40-7.33 (m, 2H), |
| | | | 7.33-7.29 (m, 2H), 7.27-7.22 (m, 1H), 7.00 (s, |
| | | | 1H), 6.47 (d, J = 8.4 Hz, 1H), 4.74 (q, J = 8.8 Hz, |
| | | | 1H), 4.34 (d, J = 8.4 Hz, 2H), 3.97 (d, J = 8.4 Hz, |
| | | | 2H), 3.60 (d, J = 8.8 Hz, 1H), 3.56-3.45 (m, 2H), |
| | | | 3.43-3.37 (m, 1H), 2.63 (s, 3H), 2.38 (br dd, J = |
| | | | 4.8, 7.6 Hz, 2H), 2.27 (br dd, J = 4.0, 7.2 Hz, 1H), |
| | | | 2.16 (br dd, J = 8.8, 15.8 Hz, 2H), 2.04-1.96 (m, |
| | | | 1H), 1.46 (s, 3H) |
| 229 | Similar to | m/z 539.2 | ¹H NMR (400 MHz, CHLOROFORM-d) δ = 8.10 |
| | Example 19 | [M + H]⁺ | (br d, J = 7.6 Hz, 1H), 7.91 (br t, J = 7.6 Hz, 1H), |
| | | | 7.69 (br s, 1H), 7.64-7.45 (m, 2H), 7.42-7.27 (m, |
| | | | 5H), 7.01 (s, 1H), 4.48 (br d, J = 3.6 Hz, 1H), 4.17 |
| | | | (br dd, J = 3.6, 7.4 Hz, 1H), 3.62 (br d, J = 8.8 Hz, |
| | | | 1H), 3.57-3.48 (m, 2H), 3.45-3.39 (m, 1H), 3.00- |
| | | | 2.93 (m, 1H), 2.89-2.81 (m, 1H), 2.65 (s, 3H), 2.41- |
| | | | 2.36 (m, 1H), 2.31-2.26 (m, 1H), 2.15-1.99 (m, |
| | | | 4H), 1.92-1.78 (m, 2H), 1.66-1.51 (m, 1H), 1.47 |
| | | | (s, 3H) |
| 230 | Similar to | m/z 570.3 | ¹H NMR (400 MHz, CHLOROFORM-d) δ = 7.68 |
| | Example 19 | [M + H]⁺ | (dd, J = 7.2, 8.4 Hz, 1H), 7.62 (s, 1H), 7.55 (d, J = |
| | | | 7.2 Hz, 1H), 7.41-7.34 (m, 2H), 7.34-7.27 (m, |
| | | | 3H), 7.25 (br d, J = 1.6 Hz, 1H), 7.07-6.98 (m, |
| | | | 2H), 4.33 (ddd, J = 3.2, 5.7, 8.5 Hz, 1H), 4.09 (br |
| | | | dd, J = 2.8, 13.0 Hz, 1H), 3.83-3.66 (m, 3H), 3.60 |
| | | | (dd, J = 3.2, 8.9 Hz, 1H), 3.57-3.46 (m, 2H), 3.45- |
| | | | 3.36 (m, 1H), 2.58 (s, 3H), 2.42-2.35 (m, 1H), 2.27 |
| | | | (ddd, J = 3.6, 7.5, 11.8 Hz, 1H), 1.97 (br dd, J = 4.4, |
| | | | 8.6 Hz, 1H), 1.83-1.80 (m, 1H), 1.47 (s, 3H), 1.42 |
| | | | (s, 3H) |
| 231 | Similar to | m/z 550.2 | ¹H NMR (400 MHz, DMSO-d₆) δ = 8.38 (br s, 1H), |
| | Example 19 | [M + H]⁺ | 7.94 (br s, 1H), 7.61 (br t, J = 7.6 Hz, 1H), 7.41- |
| | | | 7.37 (m, 2H), 7.36-7.32 (m, 2H), 7.24 (br t, J = 7.2 |
| | | | Hz, 2H), 6.69-6.42 (m, 1H), 4.76 (br s, 2H), 4.43 |
| | | | (br d, J = 0.8 Hz, 2H), 4.35-4.13 (m, 4H), 3.61 (br |
| | | | d, J = 9.6 Hz, 2H), 3.52 (br s, 2H), 2.56 (br s, 3H), |
| | | | 2.31-2.18 (m, 2H), 1.36 (s, 3H) |
| 232 | Similar to | m/z 538.2 | ¹H NMR (400 MHz, CHLOROFORM-d) δ = 7.82 |
| | Example 19 | [M + H]⁺ | (s, 1H), 7.69-7.61 (m, 2H), 7.53-7.48 (m, 1H), |
| | | | 7.40-7.36 (m, 2H), 7.35-7.28 (m, 3H), 7.06-6.99 |
| | | | (m, 1H), 6.63 (br d, J = 8.4 Hz, 1H), 4.11 (br d, J = |
| | | | 10.0 Hz, 1H), 3.89-3.84 (m, 1H), 3.77 (br d, J = |
| | | | 9.5 Hz, 2H), 3.62 (br d, J = 9.2 Hz, 1H), 3.57-3.48 |
| | | | (m, 2H), 3.46-3.40 (m, 1H), 2.63 (s, 3H), 2.42- |
| | | | 2.36 (m, 1H), 2.31-2.27 (m, 1H), 2.11-2.08 (m, |
| | | | 1H), 1.48 (s, 3H), 1.39-1.35 (m, 1H), 1.01 (br t, |
| | | | J = 4.8 Hz, 1H) |
| 233 | Similar to | m/z 570.3 | ¹H NMR (400 MHz, CHLOROFORM-d) δ = 8.27 |
| | Example 19 | [M + H]⁺ | (s, 1H), 7.69-7.55 (m, 1H), 7.40-7.32 (m, 2H), |
| | | | 7.32-7.27 (m, 2H), 7.26-7.19 (m, 1H), 7.00-6.89 |
| | | | (m, 1H), 4.23-4.06 (m, 2H), 3.92 (s, 1H), 3.88 (d, |
| | | | J = 10.0 Hz, 3H), 3.84-3.77 (m, 1H), 3.77-3.60 (m, |
| | | | 4H), 3.59-3.53 (m, 2H), 3.48 (td, J = 8.0, 15.6 Hz, |
| | | | 2H), 3.42-3.34 (m, 1H), 2.63-2.47 (m, 3H), 2.39- |
| | | | 2.30 (m, 1H), 2.28-2.20 (m, 1H), 2.14-1.93 (m, |
| | | | 4H), 1.49-1.39 (m, 3H) |
| 234 | Similar to | m/z 570.2 | ¹H NMR (400 MHz, CHLOROFORM-d) δ = 7.65 |
| | Example 19 | [M + H]⁺ | (br d, J = 3.6 Hz, 1H), 7.43-7.34 (m, 2H), 7.33- |
| | | | 7.29 (m, 2H), 7.26-7.22 (m, 1H), 7.01 (br s, 1H), |
| | | | 4.24-4.10 (m, 1H), 4.05-3.93 (m, 1H), 3.91-3.81 |
| | | | (m, 4H), 3.80-3.64 (m, 3H), 3.61 (br d, J = 9.2 Hz, |
| | | | 2H), 3.58-3.47 (m, 3H), 3.45-3.39 (m, 1H), 2.64 |
| | | | (br d, J = 4.4 Hz, 3H), 2.40-2.36 (m, 1H), 2.27 (br |
| | | | s, 1H), 2.04 (br d, J = 6.4 Hz, 4H), 1.47 (br d, J = |
| | | | 2.6 Hz, 3H) |
| 235 | Similar to | m/z 556.2 | ¹H NMR (400 MHz, METHANOL-d₄) δ = 7.81 (br |
| | Example 19 | [M + H]⁺ | d, J = 5.6 Hz, 1H), 7.41-7.29 (m, 4H), 7.29-7.16 |
| | | | (m, 2H), 4.24-4.08 (m, 1H), 4.06-3.98 (m, 1H), |
| | | | 3.82 (br s, 1H), 3.75-3.66 (m, 1H), 3.65-3.36 (m, |
| | | | 8H), 2.59 (br d, J = 8.8 Hz, 3H), 2.39-2.24 (m, |
| | | | 2H), 2.17-2.00 (m, 4H), 1.44 (d, J = 6.8 Hz, 3H) |
| 236 | Similar to | m/z 512.3 | ¹H NMR (400 MHz, CHLOROFORM-d) δ = 7.71 |
| | Example 19 | [M + H]⁺ | (br d, J = 10.0 Hz, 1H), 7.64 (br d, J = 7.6 Hz, 1H), |
| | | | 7.42-7.27 (m, 4H), 7.25 (br s, 1H), 6.98 (br d, J = |
| | | | 13.2 Hz, 1H), 4.22-4.06 (m, 1H), 4.00-3.77 (m, |
| | | | 2H), 3.76-3.37 (m, 9H), 2.60 (br d, J = 17.6 Hz, |
| | | | 3H), 2.43-2.32 (m, 1H), 2.30-2.21 (m, 1H), 2.17- |
| | | | 1.94 (m, 4H), 1.46 (d, J = 6.8 Hz, 3H) |
| 237 | Similar to | m/z 560.4 | ¹H NMR (400 MHz, CHLOROFORM-d) δ = 8.80 |
| | Example 19 | [M + H]⁺ | (br d, J = 2.0 Hz, 2H), 7.66 (s, 1H), 7.50 (br d, J = 7.2 |
| | | | Hz, 1H), 7.41-7.34 (m, 2H), 7.34-7.29 (m, 2H), |
| | | | 7.24 (br s, 1H), 7.01 (s, 1H), 5.01-4.84 (m, 1H), |
| | | | 4.07 (br dd, J = 6.4, 12.4 Hz, 1H), 3.93-3.73 (m, |
| | | | 3H), 3.61 (br d, J = 8.8 Hz, 1H), 3.57-3.45 (m, 2H), |
| | | | 3.45-3.37 (m, 1H), 3.15 (s, 3H), 2.95 (br s, 1H), |
| | | | 2.62 (s, 3H), 2.52-2.33 (m, 2H), 2.31-2.19 (m, |
| | | | 2H), 1.47 (s, 3H) |
| 238 | Similar to | m/z 541.3 | ¹H NMR (400 MHz, CHLOROFORM-d) δ = 8.61 |
| | Example 19 | [M + H]⁺ | (br d, J = 4.0 Hz, 1H), 7.66 (s, 1H), 7.44-7.29 (m, |
| | | | 5H), 7.27 (s, 2H), 7.01 (s, 1H), 4.90-4.60 (m, 2H), |
| | | | 4.51-4.31 (m, 1H), 3.61 (br d, J = 9.0 Hz, 1H), 3.57- |
| | | | 3.45 (m, 2H), 3.45-3.37 (m, 1H), 3.24 (br t, J = |
| | | | 12.6 Hz, 2H), 2.62 (s, 3H), 2.44-2.34 (m, 1H), 2.31- |
| | | | 2.19 (m, 3H), 1.62 (br d, J = 11.6 Hz, 2H), 1.47 (s, |
| | | | 3H) |
| 239 | Similar to | m/z 580.3 | ¹H NMR (400 MHz, CHLOROFORM-d) δ = 7.70- |
| | Example 19 | [M + H]⁺ | 7.60 (m, 2H), 7.48 (d, J = 7.2 Hz, 1H), 7.41-7.34 |
| | | | (m, 2H), 7.34-7.28 (m, 2H), 7.25 (br s, 1H), 7.00 |
| | | | (s, 1H), 6.63 (d, J = 8.4 Hz, 1H), 3.99-3.90 (m, 2H), |
| | | | 3.89-3.78 (m, 2H), 3.67-3.56 (m, 3H), 3.55-3.47 |
| | | | (m, 2H), 3.46-3.36 (m, 3H), 2.64 (s, 3H), 2.43- |
| | | | 2.35 (m, 1H), 2.30-2.23 (m, 1H), 2.03 (br t, J = 6.4 |
| | | | Hz, 2H), 1.79 (br s, 2H), 1.74 (br d, J = 4.8 Hz, 2H), |
| | | | 1.47 (s, 3H) |
| 240 | Similar to | m/z 552.2 | ¹H NMR (400 MHz, CHLOROFORM-d) δ = 7.73- |
| | Example 19 | [M + H]⁺ | 7.61 (m, 2H), 7.51 (d, J = 7.1 Hz, 1H), 7.42-7.34 |
| | | | (m, 2H), 7.33-7.29 (m, 2H), 7.25 (br s, 1H), 7.00 |
| | | | (s, 1H), 6.65 (d, J = 8.4 Hz, 1H), 4.80-4.63 (m, 2H), |
| | | | 4.34-4.24 (m, 2H), 3.84-3.71 (m, 2H), 3.70-3.57 |
| | | | (m, 3H), 3.57-3.45 (m, 2H), 3.41 (dt, J = 3.6, 8.8 |
| | | | Hz, 1H), 2.63 (s, 3H), 2.43-2.32 (m, 3H), 2.30- |
| | | | 2.23 (m, 1H), 1.47 (s, 3H) |
| 241 | Similar to | m/z 552.3 | ¹H NMR (400 MHz, CHLOROFORM-d) δ = 7.73- |
| | Example 19 | [M + H]⁺ | 7.61 (m, 2H), 7.55 (t, J = 7.6 Hz, 1H), 7.41-7.28 (m, |
| | | | 4H), 7.27-7.23 (m, 1H), 7.01 (br d, J = 5.2 Hz, 1H), |
| | | | 6.55 (dd, J = 8.6, 10.3 Hz, 1H), 4.29 (s, 1H), 4.19- |
| | | | 4.10 (m, 2H), 4.09-4.02 (m, 3H), 4.00 (s, 1H), 3.88 |
| | | | (t, J = 7.2 Hz, 1H), 3.66-3.57 (m, 1H), 3.57-3.46 |
| | | | (m, 2H), 3.46-3.37 (m, 1H), 2.65 (d, J = 3.6 Hz, |
| | | | 3H), 2.44-2.34 (m, 1H), 2.28 (td, J = 7.2, 17.2 Hz, |
| | | | 3H), 1.47 (d, J = 5.6 Hz, 3H) |
| 242 | Similar to | m/z 582.3 | ¹H NMR (400 MHz, CHLOROFORM-d) δ = 7.68- |
| | Example 19 | [M + H]⁺ | 7.58 (m, 1H), 7.51-7.40 (m, 1H), 7.36 (br d, J = 8.0 |
| | | | Hz, 2H), 7.34-7.27 (m, 3H), 7.27-7.23 (m, 1H), |
| | | | 6.93 (br s, 1H), 6.68-6.58 (m, 1H), 4.35-4.28 (m, |
| | | | 1H), 4.05 (br d, J = 1.2 Hz, 2H), 4.01-3.91 (m, 2H), |
| | | | 3.90-3.74 (m, 3H), 3.73-3.59 (m, 3H), 3.58-3.47 |
| | | | (m, 2H), 3.46-3.30 (m, 2H), 2.65 (s, 1H), 2.49 (s, |
| | | | 2H), 2.38 (br d, J = 7.2 Hz, 1H), 2.33-2.22 (m, 3H), |
| | | | 1.47 (br d, J = 7.2 Hz, 3H) |
| 243 | Similar to | m/z 540.2 | ¹H NMR (400 MHz, DMSO-d₆) δ = 9.20 (d, J = 7.2 |
| | Example 19 | [M + H]⁺ | Hz, 1H), 8.62 (d, J = 5.8 Hz, 1H), 8.51 (d, J = 2.0 Hz, |
| | | | 1H), 7.89 (d, J = 1.6 Hz, 1H), 7.79 (dd, J = 2.0, 5.8 |
| | | | Hz, 1H), 7.42-7.31 (m, 5H), 7.26-7.21 (m, 1H), |
| | | | 4.85-4.73 (m, 1H), 4.27 (dd, J = 7.6, 10.3 Hz, 1H), |
| | | | 3.91 (dd, J = 4.0, 10.3 Hz, 1H), 3.61-3.58 (m, 1H), |
| | | | 3.54-3.48 (m, 3H), 3.06-3.01 (m, 1H), 2.77 |
| | | | (dd, J = 4.8, 17.5 Hz, 1H), 2.55 (s, 3H), 2.26 (br t, J = |
| | | | 6.8 Hz, 2H), 1.36 (s, 3H) |
| 244 | Similar to | m/z 570.3 | ¹H NMR (400 MHz, CHLOROFORM-d) δ = 7.72 |
| | Example 19 | [M + H]⁺ | (t, J = 7.6 Hz, 1H), 7.65 (br s, 1H), 7.57 (d, J = 7.2 Hz, |
| | | | 1H), 7.41-7.34 (m, 2H), 7.34-7.27 (m, 3H), 7.02 |
| | | | (s, 1H), 6.66 (dd, J = 3.6, 8.8 Hz, 1H), 4.63-4.55 |
| | | | (m, 1H), 4.34-4.13 (m, 2H), 4.07-3.93 (m, 3H), |
| | | | 3.83 (br dd, J = 4.8, 13.2 Hz, 1H), 3.61 (br d, J = 8.8 |
| | | | Hz, 1H), 3.58-3.46 (m, 3H), 3.42 (dt, J = 3.6, 8.8 |
| | | | Hz, 1H), 3.37-3.20 (m, 1H), 2.65 (s, 3H), 2.45- |
| | | | 2.34 (m, 1H), 2.32-2.22 (m, 1H), 1.47 (s, 3H) |
| 245 | Similar to | m/z 580.3 | ¹H NMR (400 MHz, CHLOROFORM-d) δ = 7.69 |
| | Example 19 | [M + H]⁺ | (s, 2H), 7.47 (br d, J = 7.6 Hz, 1H), 7.42-7.31 (m, |
| | | | 5H), 7.02 (s, 1H), 6.63 (br d, J = 8.4 Hz, 1H), 4.68 |
| | | | (br d, J = 7.2 Hz, 1H), 3.66-3.49 (m, 6H), 3.46- |
| | | | 3.40 (m, 1H), 2.65 (s, 3H), 2.46-2.24 (m, 5H), 1.49 |
| | | | (s, 3H) |
| 246 | Similar to | m/z 568.2 | ¹H NMR (400 MHz, CHLOROFORM-d) δ = 7.73-7.45 |
| | Example 19 | [M + H]+ | (m, 3H), 7.48-7.34 (m, 2H), 7.32 (br d, J = |
| | | | 7.3 Hz, 2H), 7.26 (br d, J = 1.5 Hz, 1H), 7.08-6.90 |
| | | | (m, 1H), 6.8-6.52 (m, 1H), 4.58-4.40 (m, 1H), |
| | | | 4.40-4.32 (m, 1H), 4.28-4.20 (m, 1H), 4.16-4.04 |
| | | | (m, 1H), 4.04-3.86 (m, 1H), 3.86-3.84 (m, 1H), |
| | | | 3.84-3.23 (m, 1H), 3.25-2.98(m, 1H), 2.62 (br s, |
| | | | 1H), 2.58-2.11 (m, 1H), 1.46 (br s, 1H) |
| 247 | Similar to | m/z 552.3 | ¹H NMR (400 MHz, CHLOROFORM-d) δ = 8.02-7.48 |
| | Example 19 | [M + H]+ | (m, 3H), 7.46-7.18 (m, 6H), 7.05-6.38 (m, |
| | | | 2H), 6.08-5.52 (m, 1H), 5.26 (br s, 1H), 5.40-5.20 |
| | | | (m, 1H), 5.31-5.18 (m, 1H), 5.32-5.04 (m, 1H), |
| | | | 5.20-4.99 (m, 1H), 5.63-4.95 (m, 1H), 4.98-4.71 |
| | | | (m, 1H), 4.65-4.46 (m, 1H), 4.60-4.24 (m, 1H), |
| | | | 4.20-3.91 (m, 1H), 3.75-3.66 (m, 1H), 3.96-3.60 |
| | | | (m, 1H), 3.40 (br d, J = 10.8 Hz, 1H), 3.72-3.31 |
| | | | (m, 1H), 3.44 (br d, J = 4.1 Hz, 1H), 3.66-3.20 (m, |
| | | | 1H), 4.03-3.12 (m, 1H), 2.98-2.46 (m, 1H), 2.76- |
| | | | 2.32 (m, 5H), 2.27 (br s, 2H), 2.08-1.82 (m, 1H), |
| | | | 1.95 (br d, J = 8.5 Hz, 2H), 1.49-1.48 (m, 1H), |
| | | | 1.48 (br s, 4H) |
| 248 | Similar to | m/z 537.2 | ¹H NMR (400 MHz, CHLOROFORM-d) δ = 7.68 |
| | Example 19 | [M + H]+ | (br d, J = 1.4 Hz, 1H), 8.22-7.52 (m, 1H), 7.44- |
| | | | 7.11 (m, 1H), 7.54-7.10 (m, 1H), 7.10-6.81 (m, |
| | | | 1H), 7.00 (br d, J = 5.1 Hz, 1H), 6.06-5.40 (m, |
| | | | 1H), 6.20-5.40 (m, 1H), 4.78-4.08 (m, 1H), 4.16- |
| | | | 3.68 (m, 1H), 3.70-3.32 (m, 1H), 4.84-3.18 (m, |
| | | | 1H), 2.88-2.48 (m, 1H), 2.80-2.20 (m, 1H), 2.48- |
| | | | 2.20 (m, 1H), 2.16-1.90 (m, 1H), 2.18-1.90 (m, |
| | | | 1H), 1.48 (d, J = 5.3 Hz, 1H), 1.71-1.20 (m, 1H) |
| 249 | Similar to | m/z 620.4 | ¹H NMR (400 MHz, CHLOROFORM-d) δ = 7.96- |
| | Example 19 | [M + H]+ | 7.49 (m, 1H), 7.46-7.21 (m, 1H), 7.2-6.96 (m, |
| | | | 1H), 7.04 (s, 1H), 6.78-6.56 (m, 1H), 6.69 (dd, J = |
| | | | 2.6, 8.4 Hz, 1H), 4.68-4.39 (m, 1H), 4.36-3.72 |
| | | | (m, 1H), 3.70-3.26 (m, 1H), 2.66 (d, J = 2.8 Hz, |
| | | | 1H), 2.56-2.12 (m, 1H), 1.48 (s, 1H) |
| 250 | Similar to | m/z 550.2 | ¹H NMR (400 MHz, CHLOROFORM-d) δ = 8.36-8.20 |
| | Example 19 | [M + H]+ | (m, 1H), 8.36 (s, 2H), 8.80-8.16 (m, 1H), |
| | | | 8.10-7.95 (m, 1H), 8.04 (br t, J = 7.1 Hz, 1H), |
| | | | 8.16-7.78 (m, 1H), 7.68-7.58 (m, 1H), 7.64 (s, 1H), |
| | | | 7.96-7.52 (m, 1H), 8.52-7.52 (m, 1H), 7.38-7.24 |
| | | | (m, 5H), 7.42-7.08 (m, 1H), 6.96 (br s, 2H), 7.06- |
| | | | 6.79 (m, 1H), 4.84-4.84 (m, 1H), 5.45-4.78 (m, |
| | | | 1H), 5.76-4.48 (m, 1H), 3.60-3.52 (m, 1H), 3.56 |
| | | | (br d, J = 8.3 Hz, 1H), 3.52-3.42 (m, 1H), 3.50- |
| | | | 3.42 (m, 1H), 3.50-3.40 (m, 1H), 3.40-3.34 (m, |
| | | | 1H), 3.38-3.34 (m, 1H), 3.42-3.30 (m, 1H), 3.68- |
| | | | 3.28 (m, 1H), 4.34-2.96 (m, 1H), 2.60 (s, 3H), 2.55 |
| | | | (br d, J = 3.1 Hz, 1H), 2.39-2.28 (m, 1H), 2.24- |
| | | | 2.16 (m, 2H), 2.24-2.12 (m, 1H), 2.60-1.98 (m, |
| | | | 1H), 1.40 (s, 4H), 1.44 (br s, 1H), 2.08-0.92 (m, |
| | | | 1H), 1.70-0.67 (m, 1H), 8.93-−0.50 (m, 1H) |
| 251 | Similar to | m/z 554.2 | ¹H NMR (400 MHz, CHLOROFORM-d) δ = 8.29 |
| | Example 19 | [M + H]⁺ | (s, 1H), 7.63 (br d, J = 5.6 Hz, 2H), 7.42-7.34 (m, |
| | | | 3H), 7.33-7.29 (m, 2H), 7.24 (br s, 1H), 7.00 (s, |
| | | | 1H), 6.60 (br s, 1H), 4.50-4.37 (m, 1H), 4.19 (br d, |
| | | | J = 12.8 Hz, 2H), 3.60 (br d, J = 9.2 Hz, 1H), 3.56- |
| | | | 3.46 (m, 2H), 3.44-3.38 (m, 1H), 3.37-3.28 (m, |
| | | | 2H), 2.72 (br s, 3H), 2.61 (s, 3H), 2.42-2.34 (m, |
| | | | 1H), 2.32-2.22 (m, 3H), 1.77-1.62 (m, 2H), 1.46 |
| | | | (s, 3H) |
| 252 | Similar to | m/z 580.3 | ¹H NMR (400 MHz, CHLOROFORM-d) δ = 7.66 |
| | Example 19 | [M + H]⁺ | (br s, 1H), 7.50 (br s, 1H), 7.40-7.34 (m, 2H), 7.31 |
| | | | (br d, J = 7.2 Hz, 2H), 7.27 (s, 2H), 6.98 (br s, 1H), |
| | | | 6.59 (br s, 1H), 4.55-3.76 (m, 4H), 3.74-3.27 (m, |
| | | | 8H), 2.96-2.46 (m, 3H), 2.37 (br s, 1H), 2.27 (br d, |
| | | | J = 1.6 Hz, 1H), 1.47 (br s, 3H), 1.25 (br d, J = 12.0 |
| | | | Hz, 6H) |
| 253 | Similar to | m/z 513.3 | ¹H NMR (400 MHz, DMSO-d₆) δ = 7.93 (br s, |
| | Example 19 | [M + H]⁺ | 1H), 7.44-7.33 (m, 4H), 7.31 (br s, 1H), 7.28-7.21 |
| | | | (m, 1H), 7.11 (br s, 1H), 3.99-3.83 (m, 1H), 3.71 |
| | | | (s, 1H), 3.65-3.58 (m, 2H), 3.56-3.44 (m, 4H), |
| | | | 3.28-3.17 (m, 4H), 2.54 (s, 3H), 2.27 (br t, J = 6.4 |
| | | | Hz, 2H), 1.97-1.77 (m, 4H), 1.36 (s, 3H) |
| 254 | Similar to | m/z 541.6 | ¹H NMR (400 MHz, DMSO-d₆) δ = 8.40 (br d, J = |
| | Example 19 | [M + H]⁺ | 8.4 Hz, 1H), 8.03-7.92 (m, 2H), 7.90 (s, 1H), 7.69 |
| | | | (br d, J = 7.2 Hz, 1H), 7.44-7.29 (m, 5H), 7.28- |
| | | | 7.18 (m, 1H), 4.45 (br d, J = 11.2 Hz, 1H), 4.16- |
| | | | 4.00 (m, 2H), 3.64-3.58 (m, 1H), 3.57-3.47 (m, |
| | | | 3H), 3.41-3.37 (m, 1H), 2.57 (s, 3H), 2.31-2.21 |
| | | | (m, 2H), 2.17 (br d, J = 12.4 Hz, 1H), 2.09-1.92 |
| | | | (m, 2H), 1.70-1.55 (m, 1H), 1.36 (s, 3H) |
| 255 | Similar to | m/z 595.4 | ¹H NMR (400 MHz, DMSO-d₆) δ = 7.92 (br d, J = |
| | Example 19 | [M + H]⁺ | 6.8 Hz, 1H), 7.74-7.53 (m, 2H), 7.42-7.31 (m, |
| | | | 4H), 7.31-7.19 (m, 4H), 6.59 (br dd, J = 3.2, 7.2 |
| | | | Hz, 1H), 4.31 (br d, J = 12.0 Hz, 1H), 4.15 (br dd, |
| | | | J = 6.8, 11.6 Hz, 1H), 4.06 (br d, J = 13.2 Hz, 1H), |
| | | | 4.01-3.89 (m, 1H), 3.89-3.77 (m, 2H), 3.76-3.65 |
| | | | (m, 2H), 3.63-3.53 (m, 2H), 3.53-3.46 (m, 2H), |
| | | | 3.43 (br d, J = 5.2 Hz, 1H), 3.37 (s, 2H), 2.53 (s, |
| | | | 3H), 2.25 (br t, J = 6.8 Hz, 2H), 1.34 (s, 3H) |
| 256 | Similar to | m/z 460.1 | ¹H NMR (400 MHz, DMSO-d6) δ = 9.20 (s, 1H), |
| | Example 19 | [M + H]⁺ | 8.75 (d, J = 8.0 Hz, 1H), 7.88 (s, 1H), 7.77 (d, J = |
| | | | 4.8 Hz, 1H), 7.42-7.32 (m, 5H), 7.27 (br d, J = 4.8 |
| | | | Hz, 2H), 4.45 (td, J = 8.0, 16.2 Hz, 5H), 3.62-3.58 |
| | | | (m, 1H), 3.56-3.47 (m, 2H), 3.41-3.35 (m, 1H), |
| | | | 2.68-2.56 (m, 7H), 2.31-2.21 (m, 2H), 1.36 (s, |
| | | | 3H) |
| 257 | Similar to | m/z 566.3 | ¹H NMR (400 MHz, CHLOROFORM-d) δ = 8.41- |
| | Example 19 | [M + H]⁺ | 8.22 (m, 1H), 7.71-7.58 (m, 1H), 7.54-7.45 (m, |
| | | | 1H), 7.40-7.30 (m, 3H), 7.29 (br s, 1H), 7.27-7.23 |
| | | | (m, 1H), 7.07-6.95 (m, 1H), 6.67-6.49 (m, 1H), |
| | | | 4.23 (br d, J = 7.6 Hz, 1H), 4.11-4.00 (m, 1H), |
| | | | 3.92 (br d, J = 6.4 Hz, 1H), 3.79 (br d, J = 6.9 Hz, |
| | | | 1H), 3.77-3.36 (m, 8H), 2.64 (br d, J = 12.4 Hz, |
| | | | 3H), 2.41-2.36 (m, 2H), 2.25 (br s, 2H), 2.11 (br d, |
| | | | J = 5.8 Hz, 2H), 1.46 (d, J = 9.8 Hz, 3H) |
| 258 | Similar to | m/z 566.4 | ¹H NMR (400 MHz, CHLOROFORM-d) δ = 7.72- |
| | Example 19 | [M + H]+ | 7.59 (m, 2H), 7.48 (t, J = 6.4 Hz, 1H), 7.41-7.34 |
| | | | (m, 2H), 7.34-7.28 (m, 2H), 7.27-7.22 (m, 1H), |
| | | | 7.00 (br d, J = 9.4 Hz, 1H), 6.62 (t, J = 8.4 Hz, 1H), |
| | | | 4.27-4.14 (m, 1H), 4.02 (s, 1H), 3.92 (br t, J = 7.1 |
| | | | Hz, 1H), 3.87-3.74 (m, 1H), 3.69-3.37 (m, 8H), |
| | | | 2.64 (d, J = 10.8 Hz, 3H), 2.42-2.35 (m, 1H), |
| | | | 2.31-2.24 (m, 1H), 2.21-2.14 (m, 1H), 2.14-2.09 (m, |
| | | | 2H), 2.08-1.98 (m, 2H), 1.46 (d, J = 7.6 Hz, 3H) |
| 259 | Similar to | m/z 568.2 | ¹H NMR (400 MHz, CHLOROFORM-d) δ = 7.72- |
| | Example 19 | [M + H]+ | 7.65 (m, 1H), 7.62 (s, 1H), 7.57 (d, J = 7.2 Hz, 1H), |
| | | | 7.41-7.34 (m, 2H), 7.34-7.29 (m, 2H), 7.29-7.27 |
| | | | (m, 1H), 7.02 (s, 1H), 6.63 (d, J = 8.4 Hz, 1H), 4.30 |
| | | | (br s, 4H), 4.13 (s, 2H), 4.08-3.99 (m, 2H), 3.68 |
| | | | (br d, J = 4.4 Hz, 2H), 3.61 (d, J = 8.8 Hz, 1H), |
| | | | 3.57-3.46 (m, 2H), 3.41 (dt, J = 3.6, 8.8 Hz, 1H), 2.63 |
| | | | (s, 3H), 2.42-2.37 (m, 1H), 2.28 (br dd, J = 4.0, 8.1 |
| | | | Hz, 1H), 1.47 (s, 3H) |
| 260 | Similar to | m/z 552.2 | ¹H NMR (400 MHz, CHLOROFORM-d) δ = 8.14- |
| | Example 19 | [M + H]+ | 8.02 (m, 1H), 7.98-7.84 (m, 1H), 7.73 (br d, J = |
| | | | 7.6 Hz, 1H), 7.63 (br s, 1H), 7.42-7.34 (m, 2H), |
| | | | 7.34-7.29 (m, 2H), 7.27-7.23 (m, 1H), 7.02-6.93 |
| | | | (m, 1H), 5.15-4.99 (m, 1H), 4.79 (br d, J = 11.6 |
| | | | Hz, 1H), 4.70-4.55 (m, 1H), 4.41-4.30 (m, 1H), |
| | | | 4.29-4.07 (m, 2H), 3.65-3.34 (m, 6H), 2.62 (br s, |
| | | | 3H), 2.57 (br d, J = 6.0 Hz, 2H), 2.37 (br d, J = 8.6 |
| | | | Hz, 1H), 2.29-2.23 (m, 1H), 1.47-1.46 (m, 1H), |
| | | | 1.46 (s, 2H) |
| 261 | Similar to | m/z 568.4 | ¹H NMR (400 MHz, CHLOROFORM-d) δ = 7.71- |
| | Example 19 | [M + H]+ | 7.62 (m, 2H), 7.49 (d, J = 7.2 Hz, 1H), 7.41-7.34 |
| | | | (m, 2H), 7.34-7.30 (m, 2H), 7.29 (s, 1H), 7.01 (s, |
| | | | 1H), 6.98-6.93 (m, 1H), 4.41-4.23 (m, 2H), 4.03- |
| | | | 3.94 (m, 1H), 3.61 (d, J = 8.9 Hz, 1H), 3.58-3.47 |
| | | | (m, 2H), 3.42 (dt, J = 3.8, 8.8 Hz, 1H), 3.20-3.10 |
| | | | (m, 1H), 2.97 (d, J = 13.6 Hz, 1H), 2.64 (s, 3H), |
| | | | 2.39 (td, J = 8.4, 12.2 Hz, 1H), 2.31-2.22 (m, 1H), |
| | | | 2.01 (br dd, J = 3.6, 13.6 Hz, 1H), 1.89-1.75 (m, |
| | | | 2H), 1.47 (s, 3H), 1.33-1.23 (m, 1H), 1.12 (s, 3H), |
| | | | 1.04 (s, 3H) |
| 262 | Similar to | m/z 457.2 | ¹H NMR (400 MHz, DMSO-d₆) δ = 8.63-8.52 (m, |
| | Example 19 | [M + H]⁺ | 2H), 8.06-7.99 (m, 1H), 7.96 (d, J = 1.2 Hz, 1H), |
| | | | 7.45-7.31 (m, 5H), 7.27-7.20 (m, 1H), 3.64-3.62 |
| | | | (m, 1H), 3.58-3.53 (m, 3H), 2.62 (s, 3H), 2.28 (t, |
| | | | J = 7.2 Hz, 2H), 1.37 (s, 3H). |
| 263 | Similar to | m/z 554.4 | ¹H NMR (400 MHz, CHLOROFORM-d) δ = 8.40 |
| | Example 19 | [M + H]+ | (d, J = 6.0 Hz, 1H), 8.25 (s, 1H), 7.66 (d, J = 1.2 |
| | | | Hz, 1H), 7.59 (d, J = 2.4 Hz, 1H), 7.40-7.34 (m, |
| | | | 2H), 7.34-7.29 (m, 3H), 7.27-7.24 (m, 1H), 7.01 |
| | | | (s, 1H), 6.82 (dd, J = 2.4, 5.9 Hz, 1H), 4.45-4.31 |
| | | | (m, 1H), 4.03 (br s, 1H), 3.99 (s, 3H), 3.61 (d, J = |
| | | | 8.8 Hz, 1H), 3.56-3.46 (m, 2H), 3.44-3.39 (m, |
| | | | 1H), 3.17 (br t, J = 11.6 Hz, 3H), 2.62 (s, 3H), 2.39 |
| | | | (td, J = 8.0, 12.1 Hz, 1H), 2.31-2.18 (m, 3H), 1.67 |
| | | | (dq, J = 3.2, 11.5 Hz, 2H), 1.47 (s, 3H) |
| 264 | Similar to | m/z 540.3 | ¹H NMR (400 MHz, CHLOROFORM-d) δ = 8.40 |
| | Example 19 | [M + H]+ | (br d, J = 6.4 Hz, 1H), 7.75 (br s, 1H), 7.64 (s, 1H), |
| | | | 7.47-7.33 (m, 3H), 7.33-7.28 (m, 2H), 7.27-7.22 |
| | | | (m, 1H), 7.00 (s, 1H), 6.86 (br d, J = 5.2 Hz, 1H), |
| | | | 4.49-4.40 (m, 1H), 4.23 (br d, J = 13.2 Hz, 2H), |
| | | | 3.59 (br d, J = 8.8 Hz, 1H), 3.56-3.44 (m, 2H), |
| | | | 3.44-3.29 (m, 3H), 2.61 (s, 3H), 2.38 (td, J = 8.4, |
| | | | 12.1 Hz, 1H), 2.32-2.21 (m, 3H), 1.81-1.64 (m, |
| | | | 2H), 1.46 (s, 3H) |
| 265 | Similar to | m/z 540.2 | ¹H NMR (400 MHz, CHLOROFORM-d) δ = 8.65 |
| | Example 19 | [M + H]+ | (br s, 1H), 7.81 (br d, J = 7.8 Hz, 1H), 7.66 (br s, |
| | | | 1H), 7.58 (br d, J = 4.0 Hz, 1H), 7.46-7.34 (m, |
| | | | 3H), 7.34-7.29 (m, 2H), 7.27-7.21 (m, 1H), 7.02 |
| | | | (br s, 1H), 4.35 (br d, J = 6.0 Hz, 1H), 3.61 (br d, |
| | | | J = 8.8 Hz, 1H), 3.57-3.46 (m, 2H), 3.43 (br d, J = |
| | | | 5.2 Hz, 1H), 3.33 (br d, J = 10.4 Hz, 2H), 3.23- |
| | | | 3.05 (m, 2H), 2.64 (s, 3H), 2.47-2.19 (m, 4H), |
| | | | 2.10-1.87 (m, 2H), 1.47 (s, 3H) |
| 266 | Similar to | m/z 567.2 | ¹H NMR (400 MHz, CHLOROFORM-d) δ = 8.51- |
| | Example 19 | [M + H]+ | 7.95 (m, 1H), 7.59 (br s, 1H), 7.35-7.27 (m, 3H), |
| | | | 7.22 (br d, J = 3.6 Hz, 2H), 6.94 (br s, 1H), 6.53 (br |
| | | | s, 1H), 4.21-3.91 (m, 2H), 3.45 (br s, 8H), 3.34 (br |
| | | | s, 2H), 2.54 (br s, 3H), 2.33-2.25 (m, 1H), 2.20 (br |
| | | | d, J = 2.0 Hz, 1H), 2.13-1.81 (m, 4H), 1.48-1.31 |
| | | | (m, 3H) |
| 267 | Similar to | m/z 566.3 | ¹H NMR (400 MHz, CHLOROFORM-d) δ = 7.73- |
| | Example 19 | [M + H]+ | 7.59 (m, 2H), 7.48 (t, J = 6.4 Hz, 1H), 7.41-7.28 |
| | | | (m, 4H), 7.27-7.22 (m, 1H), 7.00 (br d, J = 10.0 |
| | | | Hz, 1H), 6.62 (t, J = 8.4 Hz, 1H), 4.21 (br d, J = 5.4 |
| | | | Hz, 1H), 4.02 (s, 1H), 3.92 (br t, J = 7.2 Hz, 1H), |
| | | | 3.88-3.74 (m, 1H), 3.72-3.55 (m, 4H), 3.54-3.45 |
| | | | (m, 3H), 3.45-3.36 (m, 1H), 2.64 (br d, J = 11.2 |
| | | | Hz, 3H), 2.44-2.33 (m, 1H), 2.31-2.22 (m, 1H), |
| | | | 2.20-1.99 (m, 4H), 1.47 (d, J = 7.6 Hz, 3H) |
| 268 | Similar to | m/z 566.3 | ¹H NMR (400 MHz, CHLOROFORM-d) δ = 7.74- |
| | Example 19 | [M + H]+ | 7.57 (m, 2H), 7.49 (t, J = 6.8 Hz, 1H), 7.42-7.28 |
| | | | (m, 4H), 7.27-7.23 (m, 1H), 7.01 (br d, J = 10.0 |
| | | | Hz, 1H), 6.63 (t, J = 8.4 Hz, 1H), 4.20 (br s, 1H), |
| | | | 4.02 (s, 1H), 3.92 (br t, J = 7.2 Hz, 1H), 3.88-3.74 |
| | | | (m, 1H), 3.72-3.55 (m, 4H), 3.55-3.45 (m, 3H), |
| | | | 3.45-3.37 (m, 1H), 2.65 (br d, J = 11.6 Hz, 3H), |
| | | | 2.43-2.34 (m, 1H), 2.31-2.23 (m, 1H), 2.18-2.01 |
| | | | (m, 4H), 1.47 (d, J = 7.6 Hz, 3H) |
| 269 | Similar to | m/z 555.3 | ¹H NMR (400 MHz, CHLOROFORM-d) δ = 8.4 |
| | Example 19 | [M + H]+ | (br s, 1H), 8.00 (br s, 1H), 7.81 (br d, J = 5.6 Hz, |
| | | | 1H), 7.65 (br s, 1H), 7.47-7.29 (m, 5H), 7.26 (br s, |
| | | | 1H), 7.01 (br s, 1H), 4.22 (br dd, J = 2.4, 3.3 Hz, |
| | | | 1H), 3.65-3.32 (m, 5H), 2.62 (br s, 3H), 2.41-2.33 |
| | | | (m, 1H), 2.26 (br s, 1H), 2.11 (br s, 4H), 2.02-1.78 |
| | | | (m, 4H), 1.46 (br s, 3H) |
| 270 | Similar to | m/z 566.3 | ¹H NMR (400 MHz, CHLOROFORM-d) δ = 8.23 |
| | Example 19 | [M + H]+ | (br s, 1H), 7.65 (br d, J = 9.6 Hz, 1H), 7.39-7.29 |
| | | | (m, 3H), 7.24 (br s, 2H), 7.11 (br d, J = 1.5 Hz, 1H), |
| | | | 6.99 (br d, J = 8.4 Hz, 2H), 4.18 (br s, 1H), 3.99 (br |
| | | | s, 1H), 3.93-3.72 (m, 2H), 3.67-3.36 (m, 8H), |
| | | | 2.63 (br d, J = 10.9 Hz, 3H), 2.40-2.32 (m, 1H), |
| | | | 2.29-2.22 (m, 1H), 2.17-1.92 (m, 4H), 1.51-1.34 |
| | | | (m, 3H) |
| 271 | Similar to | m/z 526.3 | ¹H NMR (400 MHz, CHLOROFORM-d) δ = 8.02 |
| | Example 19 | [M + H]⁺ | (br d, J = 8.4 Hz, 1H), 7.90 (br s, 1H), 7.65 (s, 1H), |
| | | | 7.59 (br s, 1H), 7.40-7.34 (m, 2H), 7.34-7.29 (m, |
| | | | 2H), 7.27-7.20 (m, 1H), 7.01 (s, 1H), 6.91 (br d, J = |
| | | | 8.4 Hz, 1H), 4.97 (br d, J = 4.9 Hz, 1H), 3.85 (br |
| | | | dd, J = 6.4, 10.0 Hz, 1H), 3.72-3.46 (m, 6H), 3.41 |
| | | | (dt, J = 3.6, 8.8 Hz, 1H), 2.62 (s, 3H), 2.51 (dt, J = |
| | | | 6.8, 13.6 Hz, 1H), 2.39 (td, J = 8.4, 12.4 Hz, 1H), |
| | | | 2.35-2.22 (m, 2H), 1.47 (s, 3H) |
| 272 | Similar to | m/z 566.4 | ¹H NMR (400 MHz, CHLOROFORM-d) δ = 7.65 |
| | Example 19 | [M + H]⁺ | (s, 1H), 7.61-7.54 (m, 1H), 7.40-7.34 (m, 2H), |
| | | | 7.34-7.29 (m, 2H), 7.25 (s, 1H), 7.10 (s, 1H), 7.02 |
| | | | (s, 1H), 6.08 (br s, 1H), 4.74-4.50 (m, 1H), 4.26 |
| | | | (br s, 2H), 4.20 (br s, 2H), 3.61 (br d, J = 8.8 Hz, |
| | | | 1H), 3.57-3.46 (m, 2H), 3.45-3.35 (m, 1H), 2.83 |
| | | | (br t, J = 8.8 Hz, 2H), 2.69 (s, 3H), 2.63 (s, 3H), |
| | | | 2.41-2.36 (m, 2H), 2.30-2.24 (m, 2H), 1.47 (s, |
| | | | 3H) |
| 273 | Similar to | m/z 539.2 | ¹H NMR (400 MHz, CHLOROFORM-d) δ = 7.71 |
| | Example 19 | [M + H]⁺ | (br s, 1H), 7.69 (s, 1H), 7.58 (d, J = 7.6 Hz, 1H), |
| | | | 7.52 (br d, J = 8.3 Hz, 1H), 7.40-7.24 (m, 6H), |
| | | | 7.18 (br d, J = 8.0 Hz, 1H), 7.02 (s, 1H), 4.37-4.21 |
| | | | (m, 1H), 3.77 (br d, J = 12.4 Hz, 2H), 3.61 (d, J = |
| | | | 8.8 Hz, 1H), 3.58-3.47 (m, 2H), 3.42 (dt, J = 3.6, |
| | | | 8.8 Hz, 1H), 3.04 (br t, J = 11.6 Hz, 2H), 2.64 (s, |
| | | | 3H), 2.45-2.33 (m, 1H), 2.31-2.15 (m, 3H), 1.88- |
| | | | 1.73 (m, 2H), 1.47 (s, 3H) |
| 274 | Similar to | m/z 568.3 | ¹H NMR (400 MHz, CHLOROFORM-d) δ = 7.70- |
| | Example 19 | [M + H]⁺ | 7.62 (m, 2H), 7.52 (d, J = 7.2 Hz, 1H), 7.43 (br d, |
| | | | J = 9.2 Hz, 1H), 7.40-7.35 (m, 2H), 7.34-7.30 (m, |
| | | | 2H), 7.09-6.94 (m, 2H), 4.21 (dt, J = 3.9, 8.9 Hz, |
| | | | 1H), 4.10 (dd, J = 3.6, 13.2 Hz, 1H), 3.84 (td, J = |
| | | | 4.8, 13.6 Hz, 1H), 3.61 (d, J = 8.8 Hz, 1H), 3.58- |
| | | | 3.46 (m, 3H), 3.45-3.27 (m, 2H), 2.62 (s, 3H), 2.39 |
| | | | (td, J = 8.4, 12.0 Hz, 1H), 2.31-2.22 (m, 1H), |
| | | | 1.75-1.58 (m, 3H), 1.47 (s, 3H), 1.17 (d, J = 6.8 Hz, |
| | | | 6H) |
| 275 | Similar to | m/z 582.3 | ¹H NMR (400 MHz, METHANOL-d₄) δ = 7.84- |
| | Example 19 | [M + H]⁺ | 7.68 (m, 2H), 7.43-7.29 (m, 5H), 7.28-7.17 (m, |
| | | | 2H), 6.85 (d, J = 8.5 Hz, 1H), 4.44-4.33 (m, 1H), |
| | | | 4.27-4.19 (m, 1H), 4.17-4.11 (m, 1H), 4.10-4.01 |
| | | | (m, 1H), 3.97-3.90 (m, 1H), 3.90-3.84 (m, 1H), |
| | | | 3.84-3.73 (m, 2H), 3.69 (br d, J = 17.2 Hz, 3H), |
| | | | 3.65-3.60 (m, 1H), 3.60-3.52 (m, 3H), 3.41 (dt, |
| | | | J = 4.0, 8.8 Hz, 1H), 3.10-2.91 (m, 1H), 2.59 (s, |
| | | | 3H), 2.46-2.18 (m, 2H), 1.44 (s, 3H) |
| 276 | Similar to | m/z 540.2 | ¹H NMR (400 MHz, CHLOROFORM-d) δ = 7.75- |
| | Example 19 | [M + H]⁺ | 7.60 (m, 2H), 7.52 (d, J = 7.2 Hz, 1H), 7.42-7.34 |
| | | | (m, 2H), 7.34-7.27 (m, 3H), 7.27-7.23 (m, 1H), |
| | | | 7.03-6.90 (m, 2H), 4.45-4.34 (m, 1H), 4.30 (br d, |
| | | | J = 13.6 Hz, 2H), 3.60 (d, J = 8.8 Hz, 1H), 3.57- |
| | | | 3.45 (m, 2H), 3.41 (dt, J = 3.6, 8.7 Hz, 1H), 3.18 (br |
| | | | t, J = 12.4 Hz, 2H), 2.62 (s, 3H), 2.38 (br d, J = 12.0 |
| | | | Hz, 1H), 2.30-2.19 (m, 3H), 1.73-1.59 (m, 2H), |
| | | | 1.47 (s, 3H) |
| 277 | Similar to | m/z 526.1 | ¹H NMR (400 MHz, CHLOROFORM-d) δ = 8.59- |
| | Example 19 | [M + H]⁺ | 8.31 (m, 1H), 8.29-8.08 (m, 1H), 7.68-7.46 (m, |
| | | | 1H), 7.31 (br d, J = 6.8 Hz, 2H), 7.22 (br d, J = 7.2 |
| | | | Hz, 3H), 6.97-6.82 (m, 1H), 6.82-6.81 (m, 1H), |
| | | | 6.69-6.41 (m, 1H), 4.98-4.75 (m, 1H), 3.80 (br d, |
| | | | J = 1.2 Hz, 1H), 3.77-3.60 (m, 2H), 3.50 (br s, |
| | | | 2H), 3.43 (br s, 2H), 3.33 (br s, 1H), 2.50 (br s, 3H), |
| | | | 2.44-2.39 (m, 1H), 2.32 (br s, 2H), 2.20 (br s, 1H), |
| | | | 1.40 (br s, 3H) |
| 278 | Similar to | m/z 552.3 | ¹H NMR (400 MHz, DMSO-d₆) δ = 8.18 (s, 1H), |
| | Example 19 | [M + H]⁺ | 8.07 (br s, 1H), 7.90 (s, 1H), 7.42-7.32 (m, 4H), |
| | | | 7.30 (br s, 1H), 7.26-7.20 (m, 1H), 7.11 (s, 1H), |
| | | | 6.73 (br d, J = 3.6 Hz, 1H), 4.14 (br dd, J = 6.4, |
| | | | 11.4 Hz, 1H), 3.89-3.74 (m, 4H), 3.63-3.57 (m, |
| | | | 2H), 3.56-3.48 (m, 4H), 3.47-3.43 (m, 1H), 3.41- |
| | | | 3.33 (m, 2H), 2.53 (s, 3H), 2.25 (br t, J = 6.4 Hz, |
| | | | 2H), 1.35 (s, 3H) |
| 279 | Similar to | m/z 276.7 | ¹H NMR (400 MHz, CHLOROFORM-d) δ = 8.40- |
| | Example 19 | [M + H]⁺ | 8.28 (m, 1H), 7.77-7.54 (m, 2H), 7.40-7.31 (m, |
| | | | 3H), 7.31-7.27 (m, 1H), 7.27-7.20 (m, 1H), 7.05- |
| | | | 6.94 (m, 1H), 6.80 (br dd, J = 4.1, 15.4 Hz, 1H), |
| | | | 5.48 (br s, 1H), 4.99 (br s, 1H), 4.51-4.43 (m, 1H), |
| | | | 4.37-4.21 (m, 1H), 4.05 (br d, J = 11.8 Hz, 1H), |
| | | | 4.00-3.84 (m, 2H), 3.66 (br d, J = 7.5 Hz, 1H), |
| | | | 3.58-3.55 (m, 1H), 3.50-3.44 (m, 2H), 3.36 (br d, |
| | | | J = 13.8 Hz, 1H), 3.26 (br d, J = 11.5 Hz, 1H), 2.89 |
| | | | (br d, J = 14.8 Hz, 1H), 2.70-2.55 (m, 3H), 2.42- |
| | | | 2.31 (m, 1H), 2.31-2.13 (m, 2H), 2.02-1.92 (m, |
| | | | 1H), 1.45 (d, J = 12.5 Hz, 3H) |
| 280 | Similar to | m/z 553.2 | ¹H NMR (400 MHz, CHLOROFORM-d) δ = 7.89- |
| | Example 19 | [M + H]⁺ | 7.74 (m, 2H), 7.64 (s, 1H), 7.42-7.34 (m, 2H), 7.34- |
| | | | 7.29 (m, 2H), 7.24 (br s, 1H), 7.10-6.93 (m, 2H), |
| | | | 5.46-5.29 (m, 2H), 4.96 (br s, 1H), 4.26-4.09 (m, |
| | | | 1H), 4.05 (br dd, J = 2.4, 10.8 Hz, 1H), 3.65 (br s, |
| | | | 1H), 3.60 (br d, J = 8.8 Hz, 1H), 3.57-3.45 (m, |
| | | | 2H), 3.41 (dt, J = 3.6, 8.8 Hz, 1H), 3.10 (br s, 1H), |
| | | | 2.69-2.56 (m, 3H), 2.49-2.33 (m, 2H), 2.31-2.22 |
| | | | (m, 1H), 1.97 (br d, J = 10.4 Hz, 1H), 1.93-1.87 |
| | | | (m, 1H), 1.80 (br s, 1H), 1.46 (s, 3H) |
| 281 | Similar to | m/z 564.7 | ¹H NMR (400 MHz, CHLOROFORM-d) δ = 7.71- |
| | Example 19 | [M + H]⁺ | 7.63 (m, 2H), 7.52 (d, J = 7.2 Hz, 1H), 7.41-7.35 |
| | | | (m, 2H), 7.35-7.30 (m, 2H), 7.29 (br s, 1H), 7.25 |
| | | | (br d, J = 1.6 Hz, 1H), 7.01 (s, 1H), 6.62 (d, J = 8.4 |
| | | | Hz, 1H), 4.58 (br d, J = 12.0 Hz, 1H), 4.40 (d, J = |
| | | | 12.4 Hz, 1H), 4.12 (br d, J = 12.0 Hz, 1H), 3.92 (br |
| | | | d, J = 9.6 Hz, 2H), 3.78 (br d, J = 12.4 Hz, 1H), |
| | | | 3.63 (br d, J = 8.8 Hz, 3H), 3.59-3.47 (m, 2H), |
| | | | 3.43 (dt, J = 4.0, 8.8 Hz, 1H), 2.66 (s, 3H), 2.40 (td, |
| | | | J = 8.4, 12.0 Hz, 1H), 2.33-2.22 (m, 1H), 1.48 (s, |
| | | | 3H), 1.19 (br d, J = 5.6 Hz, 1H), 1.14 (br d, J = 5.6 |
| | | | Hz, 1H) |
| 282 | Similar to | m/z 554.2 | ¹H NMR (400 MHz, CHLOROFORM-d) δ = 8.46 |
| | Example 19 | [M + H]+ | (br s, 1H), 8.40-8.15 (m, 1H), 7.74-7.54 (m, 2H), |
| | | | 7.36 (br d, J = 7.2 Hz, 2H), 7.30 (br d, J = 7.2 Hz, |
| | | | 3H), 7.26-7.22 (m, 1H), 6.99 (br s, 1H), 6.80 (br d, |
| | | | J = 11.2 Hz, 1H), 3.92-3.81 (m, 1H), 3.60-3.45 |
| | | | (m, 5H), 3.39 (br s, 2H), 2.60 (br s, 3H), 2.54 (br s, |
| | | | 1H), 2.47-2.31 (m, 2H), 2.25 (br s, 1H), 1.87-1.71 |
| | | | (m, 2H), 1.57 (br s, 3H), 1.45 (s, 3H) |
| 283 | Similar to | m/z 573.0 | ¹H NMR (400 MHz, DMSO-d₆) δ = 8.41 (br dd, J = |
| | Example 19 | [M + H]+ | 1.6, 3.7 Hz, 1H), 8.34-8.12 (m, 1H), 7.88 (br s, |
| | | | 1H), 7.56-7.27 (m, 5H), 7.23 (br s, 2H), 7.09-6.95 |
| | | | (m, 1H), 4.56-4.25 (m, 3H), 4.11 (br s, 1H), 3.59 |
| | | | (br d, J = 8.4 Hz, 1H), 3.54-3.48 (m, 2H), 3.10 (br |
| | | | s, 3H), 3.06-2.98 (m, 2H), 2.55 (br s, 3H), 2.26 (br |
| | | | s, 2H), 1.87 (br s, 2H), 1.75-1.59 (m, 2H), 1.36 (br |
| | | | s, 3H) |
| 284 | Similar to | m/z 564.3 | ¹H NMR (400 MHz, DMSO-d₆) δ = 8.50 (d, J = 8.4 |
| | Example 19 | [M + H]+ | Hz, 1H), 8.14-8.08 (m, 1H), 7.88 (d, J = 1.2 Hz, |
| | | | 1H), 7.61 (d, J = 2.8 Hz, 1H), 7.43-7.28 (m, 5H), |
| | | | 7.27-7.20 (m, 1H), 7.16 (dd, J = 2.8, 7.3 Hz, 1H), |
| | | | 4.34-4.16 (m, 3H), 3.62-3.56 (m, 1H), 3.55-3.48 |
| | | | (m, 2H), 3.41-3.35 (m, 2H), 3.33-3.30 (m, 1H), |
| | | | 2.54 (s, 3H), 2.30-2.21 (m, 2H), 1.96 (br d, J = |
| | | | 10.4 Hz, 2H), 1.82-1.69 (m, 2H), 1.35 (s, 3H) |
| 285 | Similar to | m/z 564.4 | ¹H NMR (400 MHz, DMSO-d₆) δ = 8.37 (d, J = 8.4 |
| | Example 19 | [M + H]+ | Hz, 1H), 7.83 (d, J = 1.5 Hz, 1H), 7.69 (dd, J = 7.4, |
| | | | 8.5 Hz, 1H), 7.39-7.23 (m, 6H), 7.21-7.15 (m, |
| | | | 1H), 7.02 (d, J = 8.8 Hz, 1H), 4.53 (br d, J = 13.0 |
| | | | Hz, 2H), 4.15-4.03 (m, 1H), 3.59-3.51 (m, 1H), |
| | | | 3.50-3.40 (m, 2H), 3.37-3.32 (m, 1H), 2.95 (br t, |
| | | | J = 11.8 Hz, 2H), 2.50 (s, 3H), 2.25-2.16 (m, 2H), |
| | | | 1.84 (br d, J = 10.4 Hz, 2H), 1.65 (dq, J = 3.8, 11.9 |
| | | | Hz, 2H), 1.31 (s, 3H) |
| 286 | Similar to | m/z 568.3 | ¹H NMR (400 MHz, DMSO-d₆) δ = 7.96-7.79 (m, |
| | Example 19 | [M + H]+ | 1H), 7.75-7.60 (m, 1H), 7.43-7.30 (m, 5H), 7.29- |
| | | | 7.15 (m, 2H), 6.75-6.58 (m, 1H), 4.10 (br d, J = |
| | | | 7.8 Hz, 1H), 4.02 (br d, J = 8.8 Hz, 1H), 3.97-3.82 |
| | | | (m, 4H), 3.79-3.64 (m, 4H), 3.59 (br s, 1H), 3.55- |
| | | | 3.45 (m, 3H), 2.54 (s, 1H), 2.40 (s, 2H), 2.30-2.19 |
| | | | (m, 2H), 1.34 (br d, J = 12.4 Hz, 3H) |
| 287 | Similar to | m/z 568.3 | ¹H NMR (400 MHz, DMSO-d₆) δ = 7.81 (s, 1H), |
| | Example 19 | [M + H]+ | 7.66-7.57 (m, 1H), 7.43-7.32 (m, 4H), 7.31-7.20 |
| | | | (m, 3H), 7.05 (d, J = 8.8 Hz, 1H), 4.57 (br d, J = |
| | | | 13.4 Hz, 1H), 4.37 (br t, J = 12.8 Hz, 3H), 4.17 (br |
| | | | s, 1H), 4.03 (br s, 1H), 3.64-3.56 (m, 2H), 3.54- |
| | | | 3.45 (m, 3H), 3.37-3.35 (m, 1H), 3.22-3.16 (m, |
| | | | 2H), 2.52-2.50 (m, 3H), 2.26 (br t, J = 6.8 Hz, 2H), |
| | | | 1.36 (s, 3H) |
| 288 | Similar to | m/z 554.3 | ¹H NMR (400 MHz, DMSO-d₆) δ = 8.33 (s, 1H), |
| | Example 19 | [M + H]+ | 7.84 (s, 1H), 7.58 (t, J = 7.6 Hz, 1H), 7.45-7.31 |
| | | | (m, 4H), 7.30-7.16 (m, 3H), 6.73 (br d, J = 8.4 Hz, |
| | | | 1H), 3.72 (br s, 4H), 3.66-3.56 (m, 5H), 3.53-3.46 |
| | | | (m, 2H), 3.40-3.33 (m, 1H), 2.49 (br s, 3H), 2.25 |
| | | | (br t, J = 6.8 Hz, 2H), 1.99 (br s, 2H), 1.86 (br s, |
| | | | 2H), 1.35 (s, 3H) |
| 289 | Similar to | m/z 571.3 | |
| | Example 19 | [M + H]⁺ | |
| 290 | Similar to | m/z 501.3 | |
| | Example 19 | [M + H]⁺ | |

### Example 20

### 6-(9-(8-methyl-6-((R)-3-methyl-3-phenylpyrrolidin-1-yl)-[1,2,4]triazolo[1,5-a]pyridine-2-carboxamido)-2-oxa-6-azaspiro[3.5]nonan-6-yl)picolinic acid (Compound 20)

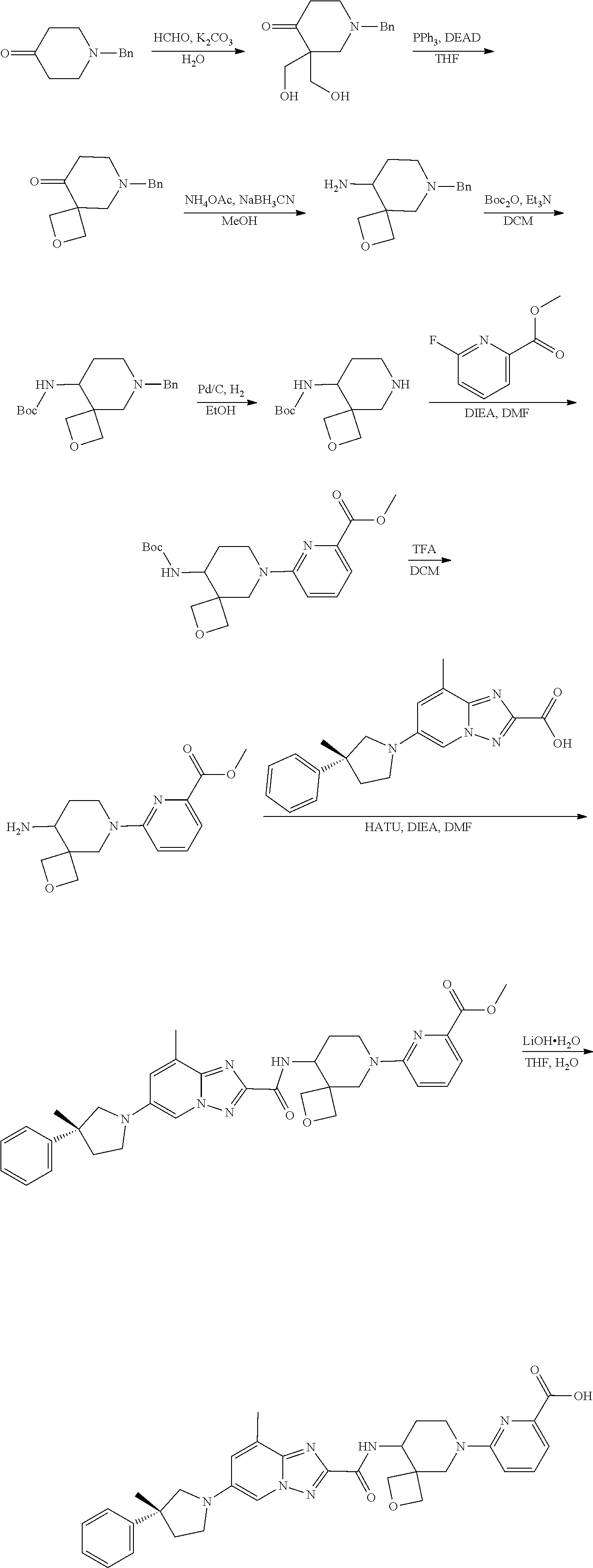

### Step 1: 1-benzyl-3,3-bis(hydroxymethyl)piperidin-4-one

To a solution of 1-benzylpiperidin-4-one (20 g, 105.68 mmol, 19.59 mL, 1 eq) in THF (100 mL) was added HCHO (13.08 g, 161.18 mmol, 12 mL, 37% purity, 1.53 eq) This was cooled with ice, and an aqueous solution H₂O (200 mL) of K₂CO₃ (2.12 g, 15.38 mmol, 1.45e-1 eq) was added thereto. The mixture was stirred at 0-25° C. for 3 h. LCMS showed starting material was consumed and one main peak with desired mass was detected. The reaction mixture was extracted with EtOAc (150 mL×3). The combined organic phase was washed with brine (200 mL), dried with anhydrous Na2SO4, filtered and concentrated in vacuum. The residue was purified by column chromatography (SiO₂, PE:EtOAc=1:3, Rf=0.50) to give compound 1-benzyl-3,3-bis(hydroxymethyl)piperidin-4-one (12 g, 41% yield) as a yellow solid.

LC-MS [ESI, M+1]: 250.1.

¹H NMR (400 MHz, CHLOROFORM-d) δ=7.34-7.15 (m, 6H), 3.74-3.63 (m, 2H), 3.61-3.54 (m, 2H), 3.52 (s, 2H), 3.03-2.84 (m, 1H), 2.75-2.68 (m, 2H), 2.63-2.53 (m, 3H), 2.51-2.32 (m, 1H).

### Step 2: 6-benzyl-2-oxa-6-azaspiro[3.5]nonan-9-one

To a solution of 1-benzyl-3,3-bis(hydroxymethyl)piperidin-4-one (5 g, 20.06 mmol, 1 eq) in Tol. (40 mL) was added PPh₃ (7.89 g, 30.08 mmol, 1.5 eq), Zinc N, n-dimethyl dithiocarbamate (6.13 g, 20.06 mmol, 1 eq) and DEAD (5.24 g, 30.08 mmol, 5.47 mL, 1.5 eq) The mixture was stirred at 25° C. for 12 h. LCMS showed starting material was consumed and one main peak with desired mass was detected. The reaction mixture was concentrated under the reduced pressure to give the crude product. The residue was purified by column chromatography (SiO₂, PE:EtOAc=3:1, Rf=0.4) to give compound 8-benzyl-2-oxa-8-azaspiro[3.5]nonan-5-one (5 g, 75% yield) as a yellow oil.

LC-MS [ESI, M+1]: 232.1.

¹H NMR (400 MHz, CHLOROFORM-d) δ=7.38-7.30 (m, 5H), 4.86 (d, J=6.0 Hz, 2H), 4.34-4.28 (m, 2H), 3.64 (s, 2H), 2.96 (s, 2H), 2.79-2.67 (m, 2H), 2.45 (t, J=6.0 Hz, 2H).

### Step 3: 6-benzyl-2-oxa-6-azaspiro[3.5]nonan-9-amine

To a solution of 8-benzyl-2-oxa-8-azaspiro[3.5]nonan-5-one (2.8 g, 12.11 mmol, 1 eq) in ammonia (30 mL) was added ZnCl₂ (165.01 mg, 1.21 mmol, 56.76 μL, 0.1 eq) and sodium;cyanoboranuide (1.52 g, 24.21 mmol, 2 eq). The mixture was stirred at 25° C. for 2 h. LCMS showed starting material was consumed and one main peak with desired mass was detected. The reaction mixture was concentrated under the reduced pressure to give the crude product. The residue was purified by column chromatography (SiO₂, DCM:MeOH=15:1, Rf=0.50) to give compound 8-benzyl-2-oxa-8-azaspiro[3.5]nonan-5-amine (2.2 g, 63% yield) as a colorless oil.

LC-MS [ESI, M+1]: 233.0.

¹H NMR (400 MHz, METHANOL-d4) δ=7.35 (s, 5H), 4.63 (d, J=7.2 Hz, 1H), 4.56 (d, J=7.2 Hz, 1H), 4.47 (d, J=7.2 Hz, 1H), 4.35 (d, J=7.2 Hz, 1H), 3.75-3.60 (m, 2H), 3.47-3.34 (m, 2H), 3.27-3.13 (m, 1H), 2.94-2.79 (m, 1H), 2.55-2.25 (m, 2H), 2.00-1.87 (m, 2H), 1.78-1.57 (m, 1H).

### Step 4: tert-butyl (6-benzyl-2-oxa-6-azaspiro[3.5]nonan-9-yl)carbamate

To a solution of 8-benzyl-2-oxa-8-azaspiro[3.5]nonan-5-amine (600 mg, 2.58 mmol, 1 eq) in MeOH (6 mL) was added Boc₂O (1.69 g, 7.75 mmol, 1.78 mL, 3 eq) and Et3N (784.00 mg, 7.75 mmol, 1.08 mL, 3 eq). The mixture was stirred at 25° C. for 12 h. LCMS showed starting material was consumed and one main peak with desired mass was detected. The reaction mixture was extracted with EtOAc (15 mL×2). The combined organic phase was washed with brine (30 mL), dried with anhydrous Na2SO4, filtered and concentrated in vacuum. The residue was purified by column chromatography (SiO2, PE:EtOAc=3:1, Rf=0.2) to give compound tert-butyl N-(8-benzyl-2-oxa-8-azaspiro[3.5]nonan-5-yl)carbamate (500 mg, 58% yield) was obtained as a colorless oil.

LC-MS [ESI, M+1]: 333.2.

¹H NMR (400 MHz, CHLOROFORM-d) δ=7.46-7.27 (m, 5H), 4.92 (br s, 1H), 4.55-4.47 (m, 2H), 4.41 (br s, 1H), 4.17 (br d, J=5.6 Hz, 1H), 3.58 (br d, J=11.6 Hz, 2H), 3.52-3.39 (m, 1H), 3.24-2.97 (m, 1H), 2.86-2.61 (m, 1H), 2.36-2.10 (m, 2H), 1.80 (br d, J=10.0 Hz, 1H), 1.74-1.57 (m, 1H), 1.47 (s, 9H).

### Step 5: tert-butyl (2-oxa-6-azaspiro[3.5]nonan-9-yl)carbamate

To a solution of tert-butyl N-(8-benzyl-2-oxa-8-azaspiro[3.5]nonan-5-yl)carbamate (1 g, 3.01 mmol, 1 eq) in MeOH (40 mL) was added Pd/C (200 mg, 187.93 mol, 10% purity, 6.25e-2 eq) under N₂ atmosphere. The suspension was degassed and purged with H2 for 3 times. The mixture was stirred under H2 (50 Psi) at 50° C. for 24 h. LCMS showed starting material was consumed and one main peak with desired mass was detected. The reaction mixture was filtered and the filtrate was concentrated under the reduced pressure to give compound tert-butyl N-(2-oxa-8-azaspiro[3.5]nonan-5-yl)carbamate (700 mg, 86% yield) as a colorless oil.

LC-MS [ESI, M+1]: 243.2.

¹H NMR (400 MHz, CHLOROFORM-d) δ=4.95 (br d, J=7.6 Hz, 1H), 4.56 (d, J=6.4 Hz, 1H), 4.54-4.50 (m, 1H), 4.45 (br d, J=6.8 Hz, 1H), 4.28-4.16 (m, 1H), 3.69-3.57 (m, 1H), 3.38 (br d, J=12.0 Hz, 1H), 2.93 (br d, J=12.4 Hz, 1H), 2.78 (br d, J=12.0 Hz, 1H), 2.71-2.60 (m, 1H), 1.83-1.73 (m, 1H), 1.63 (br s, 1H), 1.48 (s, 9H), 1.30-1.16 (m, 1H).

### Step 6: methyl 6-(9-((tert-butoxycarbonyl)amino)-2-oxa-6-azaspiro[3.5]nonan-6-yl)picolinate

To a solution of tert-butyl N-(2-oxa-8-azaspiro[3.5]nonan-5-yl)carbamate (300 mg, 1.24 mmol, 1 eq) in DMSO (2 mL) was added methyl 6-fluoropyridine-2-carboxylate (192.06 mg, 1.24 mmol, 1 eq) and DIEA (480.03 mg, 3.71 mmol, 646.94 μL, 3 eq). The mixture was stirred at 100° C. for 1 h. LCMS showed starting material was consumed and one main peak with desired mass was detected. The reaction mixture was extracted with EtOAc (15 mL×2). The combined organic phase was washed with brine (20 mL), dried with anhydrous Na2SO4, filtered and concentrated in vacuum. The residue was purified by column chromatography (SiO₂, PE:EtOAc=1:1, Rf=0.50) to give compound methyl 6-[5-(tert-butoxycarbonylamino)-2-oxa-8-azaspiro[3.5]nonan-8-yl]pyridine-2-carboxylate (150 mg, 32% yield) as a colorless oil.

LC-MS [ESI, M+1]: 378.2.

¹H NMR (400 MHz, CHLOROFORM-d) δ=7.53 (dd, J=7.6, 8.4 Hz, 1H), 7.38 (d, J=7.2 Hz, 1H), 6.85 (d, J=8.4 Hz, 1H), 4.95-4.72 (m, 1H), 4.60-4.48 (m, 2H), 4.42 (d, J=6.4 Hz, 1H), 4.34-4.19 (m, 2H), 4.15-3.98 (m, 2H), 3.87 (s, 3H), 3.79-3.65 (m, 1H), 3.22-2.90 (m, 2H), 1.92-1.80 (m, 1H), 1.42 (s, 9H)

### Step 7: methyl 6-(9-amino-2-oxa-6-azaspiro[3.5]nonan-6-yl)picolinate

To a solution of methyl 6-[5-(tert-butoxycarbonylamino)-2-oxa-8-azaspiro[3.5]nonan-8-yl]pyridine-2-carboxylate (50 mg, 132.47 μmol, 1 eq) in DCM (0.5 mL) was added TFA (767.50 mg, 6.73 mmol, 0.5 mL, 50.81 eq). The mixture was stirred at 25° C. for 10 min. LCMS showed starting material was consumed and one main peak with desired mass was detected. The reaction mixture was concentrated under the reduced pressure to give compound methyl 6-(5-amino-2-oxa-8-azaspiro[3.5]nonan-8-yl)pyridine-2-carboxylate (50 mg, 24% yield TFA) as a yellow oil.

LC-MS [ESI, M+1]: 278.2.

### Step 8: methyl 6-(9-(8-methyl-6-((R)-3-methyl-3-phenylpyrrolidin-1-yl)-[1,2,4]triazolo[1,5-a]pyridine-2-carboxamido)-2-oxa-6-azaspiro[3.5]nonan-6-yl)picolinate

To a solution of 8-methyl-6-[rac-(3R)-3-methyl-3-phenyl-pyrrolidin-1-yl]-[1,2,4]triazolo[1,5-a]pyridine-2-carboxylic acid (15 mg, 44.59 μmol, 1 eq) in DMF (1 mL) was added methyl 6-(5-amino-2-oxa-8-azaspiro[3.5]nonan-8-yl)pyridine-2-carboxylate (26.18 mg, 66.89 μmol, 1.5 eq, TFA) HATU (20.35 mg, 53.51 μmol, 1.2 eq) and DIEA (17.29 mg, 133.77 μmol, 23.30 μL, 3 eq). The mixture was stirred at 25° C. for 1 h. LCMS showed starting material was consumed and one main peak with desired mass was detected. The reaction mixture was extracted with EtOAc (20 mL×2). The combined organic phase was washed with brine (30 mL), dried with anhydrous Na2SO4, filtered and concentrated in vacuum to give compound methyl 6-[5-[[8-methyl-6-[rac-(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylate (60 mg, crude) as a yellow oil.

LC-MS [ESI, M+1]: 596.2.

### Step 9: 6-(9-(8-methyl-6-((R)-3-methyl-3-phenylpyrrolidin-1-yl)-[1,2,4]triazolo[1,5-a]pyridine-2-carboxamido)-2-oxa-6-azaspiro[3.5]nonan-6-yl)picolinic acid

To a solution of methyl 6-[5-[[8-methyl-6-[rac-(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylate (50 mg, 83.94 μmol, 1 eq) in THF (1 mL) and H₂O (0.3 mL) was added LiOH·H₂O (7.04 mg, 167.87 μmol, 2 eq). The mixture was stirred at 25° C. for 15 min. LCMS showed starting material was consumed and one main peak with desired mass was detected. The reaction mixture was concentrated under the reduced pressure to give the crude product. The residue was purified by prep-HPLC (FA condition column: CD07-Daisogel SP-100-8-ODS-PK 150*25*10 um; mobile phase: [water(NH4HCO3)—ACN]; gradient:24%-54% B over 10 min) to give compound 6-[5-[[8-methyl-6-[rac-(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylic acid (22.37 mg, 46% yield) as a white solid.

LC-MS [ESI, M+1]: 582.3.

¹H NMR (400 MHz, CHLOROFORM-d) 6=7.79-7.70 (m, 2H), 7.68 (d, J=1.2 Hz, 1H), 7.61 (d, J=7.2 Hz, 1H), 7.42-7.35 (m, 2H), 7.35-7.30 (m, 2H), 7.29 (br s, 1H), 7.09 (d, J=8.8 Hz, 1H), 7.03 (s, 1H), 4.75 (dd, J=6.8, 13.6 Hz, 2H), 4.61 (br d, J=12.9 Hz, 1H), 4.56-4.48 (m, 1H), 4.43 (d, J=6.8 Hz, 1H), 4.37 (d, J=6.8 Hz, 1H), 4.17-4.04 (m, 1H), 3.62 (br d, J=8.8 Hz, 1H), 3.58-3.47 (m, 2H), 3.47-3.39 (m, 2H), 3.29-3.18 (m, 1H), 2.66 (s, 3H), 2.40 (td, J=8.4, 12.0 Hz, 1H), 2.32-2.21 (m, 1H), 2.15-2.03 (m, 1H), 1.72 (br d, J=4.4 Hz, 1H), 1.48 (s, 3H).

| Cmpd. No. | Synthesis | LC/MS | ¹H NMR |
|---|---|---|---|
| 291 | Similar to | m/z 564.3 | ¹H NMR (400 MHz, CD3OD, 297 K) δ (ppm) = 7.74 |
| | Example 20 | [M + H]⁺ | (m, J = 7.3, 8.6 Hz, 1H), 7.59 (d, J = 1.2 Hz, 1H), |
| | | | 7.39 (d, J = 7.2 Hz, 1H), 7.34-7.16 (m, 5H), 6.89- |
| | | | 6.81 (m, 2H), 4.32-4.22 (m, 1H), 4.05-3.78 (m, |
| | | | 6H), 3.74-3.62 (m, 3H), 3.62-3.53 (m, 2H), 3.53- |
| | | | 3.50 (m, 1H), 3.21 (d, J = 3.2 Hz, 2H), 2.54-2.46 |
| | | | (m, 3H), 2.39-2.17 (m, 4H) |

### Example 21

### 6-((R)-7-(8-methyl-6-((S)-3-methyl-3-(oxazol-2-yl)pyrrolidin-1-yl)-[1,2,4]triazolo[1,5-a]pyridine-2-carbonyl)-2,7-diazaspiro[4.4]nonan-2-yl)picolinic acid (Compound 21)

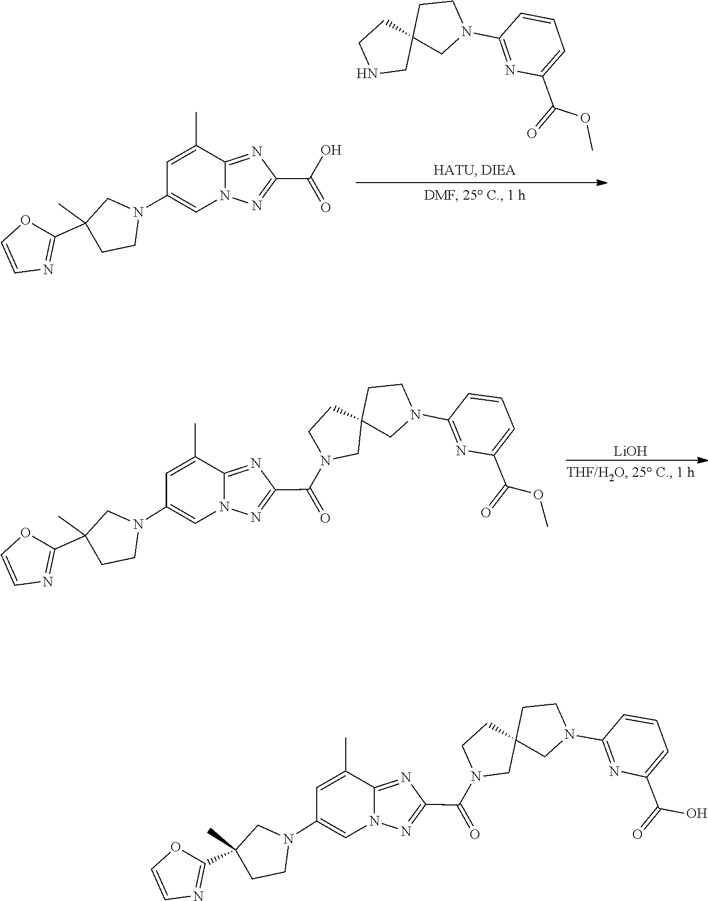 [SMILES: Cc1cc(N2CCC(C)(c3ncco3)C2)cn2nc(C(=O)O)nc12 ; COC(=O)c1cccc(N2CC[C@@]3(CCNC3)C2)n1 ; COC(=O)c1cccc(N2CC[C@@]3(CCN(C(=O)c4nc5c(C)cc(N6CCC(C)(c7ncco7)C6)cn5n4)C3)C2)n1 ; "LiOH" ; Cc1cc(N2CC[C@](C)(c3ncco3)C2)cn2nc(C(=O)N3CC[C@@]4(CCN(c5cccc(C(=O)O)n5)C4)C3)nc12]

### Step 1: methyl 6-((5R)-7-(8-methyl-6-(3-methyl-3-(oxazol-2-yl)pyrrolidin-1-yl)-[1,2,4]triazolo[1,5-a]pyridine-2-carbonyl)-2,7-diazaspiro[4.4]nonan-2-yl)picolinate

To a solution of 8-methyl-6-(3-methyl-3-oxazol-2-yl-pyrrolidin-1-yl)-[1,2,4]triazolo[1,5-a]pyridine-2-carboxylic acid (50 mg, 149.58 μmol, 1 eq, Li), methyl 6-[(5R)-2,7-diazaspiro[4.4]nonan-2-yl]pyridine-2-carboxylate (39.09 mg, 149.58 μmol, 1 eq) in DMF (2 mL) was added DIEA (96.66 mg, 747.88 μmol, 130.27 μL, 5 eq) and HATU (85.31 mg, 224.36 μmol, 1.5 eq). The mixture was stirred at 20° C. for 1 hr. The suspension was filtered and the filtrated was concentrated under reduced pressure. The residue was further purification by pre-HPLC (column: Waters Xbridge 150*25 mm*5 um; mobile phase: [water(NH4HCO3)—ACN]; gradient:35%-65% B over 9 min) and lyophilized. The title compound (20 mg, 35.05 μmol, 23.43% yield) was obtained as a white solid.

### Step 2: 6-((R)-7-(8-methyl-6-((S)-3-methyl-3-(oxazol-2-yl)pyrrolidin-1-yl)-[1,2,4]triazolo[1,5-a]pyridine-2-carbonyl)-2,7-diazaspiro[4.4]nonan-2-yl)picolinic acid

To a mixture of methyl 6-[(5R)-2-[8-methyl-6-(3-methyl-3-oxazol-2-yl-pyrrolidin-1-yl)-[1,2,4]triazolo[1,5-a]pyridine-2-carbonyl]-2,7-diazaspiro[4.4]nonan-7-yl]pyridine-2-carboxylate (20 mg, 35.05 μmol, 1 eq) in THF (0.5 mL) and H₂O (0.5 mL) and MeOH (0.5 mL) was added LiOH·H2O (4.41 mg, 105.14 μmol, 3 eq) in one portion at 15° C. The mixture was stirred at 30° C. for 1 hours. The reaction mixture was concentrated under reduced pressure to give a residue. The residue was further purification by pre-HPLC (column: YMC-Actus Triart C18 150*30 mm*7 um; mobile phase: [water(FA)—ACN]; gradient:25%-55% B over 10 min) and lyophilized. The title compound (14.93 mg, 25.62 μmol, 73.09% yield, 95.5% purity) was obtained as a white solid.

LC-MS [ESI, M+1]: 557.3.

¹H NMR (400 MHz, DMSO-d6) δ=12.43 (s, 1H), 8.09-8.02 (m, 1H), 7.93-7.83 (m, 1H), 7.69-7.55 (m, 1H), 7.30-7.18 (m, 2H), 7.16-7.09 (m, 1H), 6.73-6.61 (m, 1H), 3.95 (br t, J=7.0 Hz, 1H), 3.86-3.76 (m, 2H), 3.70-3.35 (m, 11H), 2.61-2.51 (m, 4H), 2.19-1.93 (m, 5H), 1.50 (d, J=7.6 Hz, 3H)

### Example 22

### (R)-4-((tert-butoxycarbonyl)amino)-6-(3-methyl-3-phenylpyrrolidin-1-yl)pyrazolo[1,5-a]pyridine-2-carboxylic acid (Compound 22)

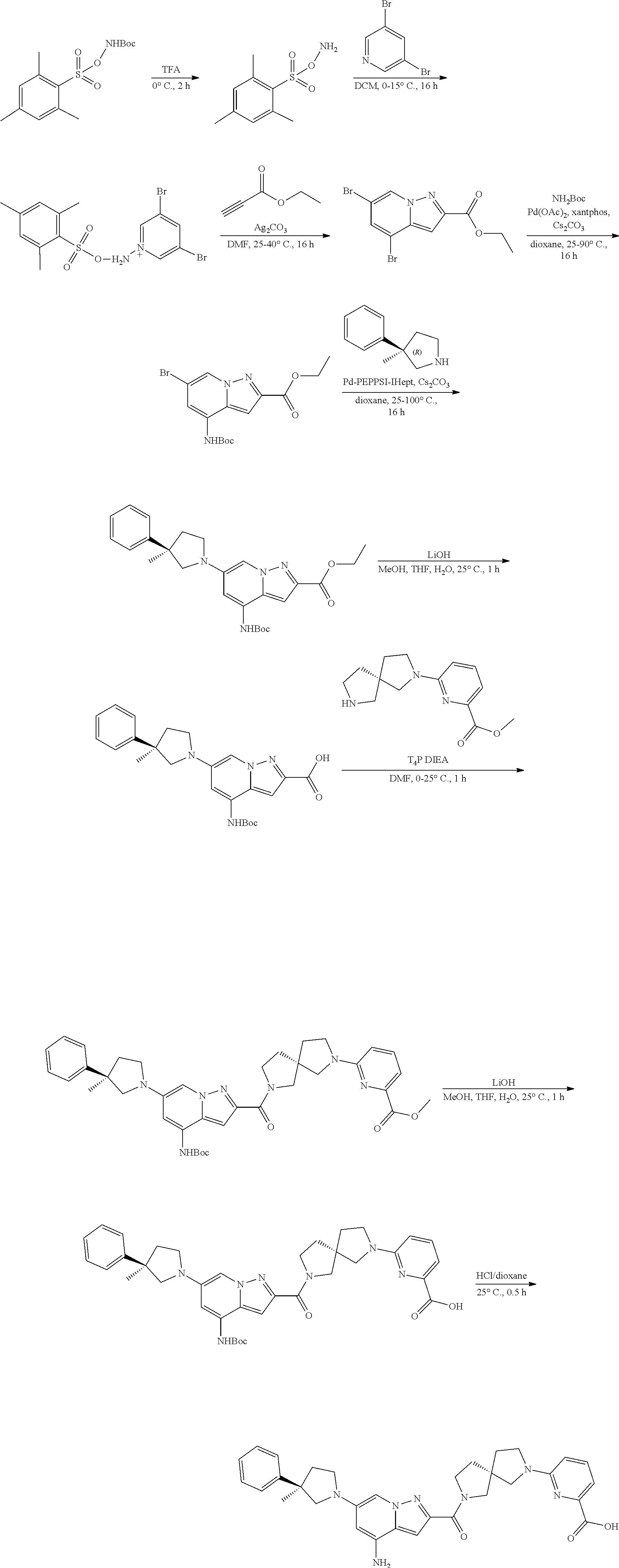

### Step 1: O-(mesitylsulfonyl)hydroxylamine

To a mixture of tert-butyl ((mesitylsulfonyl)oxy)carbamate (30 g, 95.12 mmol) in TFA (92.10 g, 807.73 mmol, 60 mL) in one portion at 0° C. The mixture was stirred at 0° C. for 2 h. The reaction mixture was poured into H₂O (0° C., 300 mL), then it was to filter to give the title compound (25 g, 61% yield) as white solid.

¹H NMR (400 MHz, CD₃OD) δ=7.11-6.82 (m, 2H), 2.69-2.52 (m, 6H), 2.34-2.21 (m, 3H)

### Step 2: 1-amino-3,5-dibromopyridin-1-ium 2,4,6-trimethylbenzenesulfonate

To a mixture of O-(mesitylsulfonyl) hydroxylamine (20 g, 92.91 mmol) in DCM (200 mL) was added 3,5-dibromopyridine (14.75 g, 62.25 mmol) in one portion at 0° C. The mixture was stirred at 25° C. for 16 h. The mixture was to filter to give the title compound (26.5 g, 79% yield) as white solid.

¹H NMR (400 MHz, CD₃OD) δ=9.02 (d, J=1.6 Hz, 2H), 8.79 (t, J=1.6 Hz, 1H), 6.87 (s, 2H), 2.62 (s, 6H), 2.24 (s, 3H)

### Step 3: ethyl 4,6-dibromopyrazolo[1,5-a]pyridine-2-carboxylate

To a mixture of 1-amino-3,5-dibromopyridin-1-ium 2,4,6-trimethylbenzenesulfonate (26.5 g, 72.95 mmol) in DMF (260 mL) was added Ag₂CO₃ (20.12 g, 72.95 mmol, 3.31 mL) and ethyl propiolate (7.16 g, 72.95 mmol, 7.16 mL) in one portion at 25° C. The mixture was stirred at 40° C. for 16 h. The reaction mixture was poured into water (1300 mL), extracted with EtOAc (1000 mL×2). The combined organic layers were washed with brine (1400 mL), dried over Na2SO4, filtered and concentrated under reduced pressure to give a residue. The residue was purified by flash silica gel chromatography (SiO₂; PE:EtOAc=20:1, Rf=0.43) to give the title compound (4.3 g, 17% yield) as yellow solid.

LC-MS [ESI, M+1]: 347.9.

1H NMR (400 MHz, CD₃OD) δ=8.87 (t, J=1.2 Hz, 1H), 7.72 (d, J=1.2 Hz, 1H), 7.16 (d, J=0.8 Hz, 1H), 4.43 (d, J=7.2 Hz, 2H), 1.42 (t, J=7.2 Hz, 3H)

### Step 4: ethyl 6-bromo-4-((tert-butoxycarbonyl)amino)pyrazolo[1,5-a]pyridine-2-carboxylate

To a solution of ethyl ethyl 4,6-dibromopyrazolo[1,5-a]pyridine-2-carboxylate (2.6 g, 7.47 mmol), NH₂Boc (1.05 g, 8.97 mmol), Cs₂CO₃ (4.87 g, 14.94 mmol), Xantphos (432.31 mg, 747.15 mol) in dioxane (20 mL) was added Pd(OAc)₂ (167.74 mg, 747.15 mol) under N₂ at 25° C. The mixture was stirred at 90° C. for 16 h. The reaction mixture was poured into water (80 mL), extracted with EtOAc (120 mL×2). The combined organic layers were washed with brine (140 mL), dried over Na2SO4, filtered and concentrated under reduced pressure to give a residue. The residue was purified by flash silica gel chromatography (SiO₂; PE:EtOAc=5:1, Rf=0.48) to give the title compound (920 mg, 32% yield) as yellow solid.

LC-MS [ESI, M+1]: 384.1.

¹H NMR (400 MHz, CDCl₃) δ=8.40 (s, 1H), 7.98 (s, 1H), 7.12 (s, 1H), 6.79 (s, 1H), 4.51-4.45 (m, 2H), 1.46 (s, 9H), 1.46-1.40 (m, 3H)

### Step 5: ethyl (R)-4-((tert-butoxycarbonyl)amino)-6-(3-methyl-3-phenylpyrrolidin-1-yl)pyrazolo[1,5-a]pyridine-2-carboxylate

To a mixture of ethyl 6-bromo-4-((tert-butoxycarbonyl)amino)pyrazolo[1,5-a]pyridine-2-carboxylate (500 mg, 1.30 mmol) and (R)-3-methyl-3-phenylpyrrolidine (209.83 mg, 1.30 mmol) in dioxane (6 mL) was added Cs₂CO₃ (847.99 mg, 2.60 mmol) and 1,3-bis[2,6-bis(1-propylbutyl)phenyl]-4,5-dichloro-2H-imidazol-1-ium-2-ide;3-chloropyridine;dichloropalladium (126.59 mg, 130.13 mol) in one portion at 25° C. under N₂. The mixture was stirred at 100° C. for 16 h. The reaction mixture was poured into water (30 mL), extracted with Ethyl acetate (80 mL×2). The combined organic layers were washed with brine (80 mL), dried over Na2SO4, filtered and concentrated under reduced pressure to give a residue. The residue was purified by flash silica gel chromatography (SiO₂; PE:EtOAc=3:1, Rf=0.55) to give the title compound (360 mg, 58% yield) as yellow solid.

LC-MS [ESI, M+1]: 465.3.

¹H NMR (400 MHz, CDCl₃) δ=7.67-7.54 (m, 2H), 7.39-7.29 (m, 4H), 7.26-7.22 (m, 1H), 6.99 (s, 1H), 6.64 (s, 1H), 4.45 (m, J=7.2 Hz, 2H), 3.62 (d, J=9.2 Hz, 1H), 3.58-3.48 (m, 2H), 3.42 (m, J=3.6, 8.8 Hz, 1H), 2.35 (m, J=8.4, 12.0 Hz, 1H), 2.27-2.16 (m, 1H), 1.57 (s, 9H), 1.45 (s, 6H)

### Step 6: (R)-4-((tert-butoxycarbonyl)amino)-6-(3-methyl-3-phenylpyrrolidin-1-yl)pyrazolo[1,5-a]pyridine-2-carboxylic acid

To a solution of ethyl (R)-4-((tert-butoxycarbonyl)amino)-6-(3-methyl-3-phenylpyrrolidin-1-yl)pyrazolo[1,5-a]pyridine-2-carboxylate (353 mg, 759.87 mol) in THF (2 mL), H₂O (1.5 mL), MeOH (1 mL) was added LiOH·H₂O (95.66 mg, 2.28 mmol). The mixture was stirred at 25° C. for 1 h. The reaction mixture was concentrated under reduced pressure to remove solvent to give the title compound (331 mg, 91% yield) as yellow solid.

LC-MS [ESI, M+1]: 437.2.

¹H NMR (400 MHz, CD₃OD) δ=7.60-7.48 (m, 2H), 7.40-7.29 (m, 4H), 7.21 (s, 1H), 6.95 (s, 1H), 3.60-3.50 (m, 3H), 3.39 (m, J=4.0, 8.8 Hz, 1H), 2.31 (d, J=9.6 Hz, 2H), 1.58 (s, 9H), 1.44 (s, 3H)

### Step 7: methyl 6-((R)-7-(4-((tert-butoxycarbonyl)amino)-6-((R)-3-methyl-3-phenylpyrrolidin-1-yl)pyrazolo[1,5-a]pyridine-2-carbonyl)-2,7-diazaspiro[4.4]nonan-2-yl)picolinate

To a solution of (R)-4-((tert-butoxycarbonyl)amino)-6-(3-methyl-3-phenylpyrrolidin-1-yl)pyrazolo[1,5-a]pyridine-2-carboxylic acid (50 mg, 114.55 mol), methyl (R)-6-(2,7-diazaspiro[4.4]nonan-2-yl)picolinate (29.93 mg, 114.55 mol) in DMF (1 mL) was added DIEA (44.41 mg, 343.64 μmol, 59.86 μL) and T4P (123.80 mg, 171.82 μmol, 50% purity) at 0° C. The mixture was stirred at 25° C. for 1 hr. The reaction mixture was poured into water (8 ml), extracted with Ethyl acetate (10 mL×2). The combined organic layers were washed with brine (15 mL), dried over Na2SO4, filtered and concentrated under reduced pressure to give a residue. The reaction mixture was poured into water (8 ml), extracted with DCM (10 mL×2). The combined organic layers were washed with brine (15 mL), dried over Na2SO4, filtered and concentrated under reduced pressure to give a residue. The residue was purified by prep-HPLC (column: YMC-Actus Triart C18 150*30 mm*7 um; mobile phase: [water(FA)—ACN]; gradient:80%-100% B over 10 min) to give the title compound (40 mg, 48% yield) as yellow solid.

LC-MS [ESI, M+1]: 680.4.

### Step 8: 6-((R)-7-(4-((tert-butoxycarbonyl)amino)-6-((R)-3-methyl-3-phenylpyrrolidin-1-yl)pyrazolo[1,5-a]pyridine-2-carbonyl)-2,7-diazaspiro[4.4]nonan-2-yl)picolinic acid

To a solution of methyl methyl 6-((R)-7-(4-((tert-butoxycarbonyl)amino)-6-((R)-3-methyl-3-phenylpyrrolidin-1-yl)pyrazolo[1,5-a]pyridine-2-carbonyl)-2,7-diazaspiro[4.4]nonan-2-yl)picolinate (40 mg, 58.84 mol) in THF (0.2 mL), MeOH (0.2 mL), H₂O (0.1 mL) was added LiOH·H₂O (7.41 mg, 176.52 mol). The mixture was stirred at 25° C. for 1 h. The reaction mixture was concentrated under reduced pressure to remove solvent to give the title compound (39 mg, 99% yield) as yellow solid.

LC-MS [ESI, M+1]: 666.4.

### Step 9: 6-((R)-7-(4-amino-6-((R)-3-methyl-3-phenylpyrrolidin-1-yl)pyrazolo[1,5-a]pyridine-2-carbonyl)-2,7-diazaspiro[4.4]nonan-2-yl)picolinic acid

A mixture of 6-((R)-7-(4-((tert-butoxycarbonyl)amino)-6-((R)-3-methyl-3-phenylpyrrolidin-1-yl)pyrazolo[1,5-a]pyridine-2-carbonyl)-2,7-diazaspiro[4.4]nonan-2-yl)picolinic acid (39 mg, 58.58 mol) in HCl/dioxane (2 M, 29.29 L) was stirred at 25° C. for 0.5 h. The reaction mixture was concentrated under reduced pressure to remove solvent. The residue was purified by prep-HPLC (column: Phenomenex luna C18 150*25 mm*10 um; mobile phase: [water(HCl)—ACN]; gradient:88%-38% B over 10 min) to give the title compound (17.01 mg, 49% yield) as orange solid

LC-MS [ESI, M+1]: 566.3.

¹H NMR (400 MHz, CD₃OD) δ=8.14-8.04 (m, 1H), 7.62-7.55 (m, 1H), 7.51 (d, J=1.6 Hz, 1H), 7.42-7.35 (m, 5H), 7.30-7.24 (m, 1H), 5.89-5.78 (m, 1H), 5.76-5.49 (m, 2H), 4.27-4.06 (m, 2H), 4.00-3.71 (m, 10H), 2.54-2.35 (m, 2H), 2.32-2.07 (m, 4H), 1.52-1.39 (m, 3H)

| Cmpd. No. | Synthesis | LC/MS | ¹H NMR |
|---|---|---|---|
| 292 | Similar to | m/z | ¹H NMR (400 MHz, CD3OD, 297 K) δ (ppm) = 8.50- |
| | Example | 350.2 | 8.38 (m, 1H), 7.48-7.19 (m, 6H), 5.89-5.73 (m, 1H), |
| | 22 | [M + H]⁺ | 3.74 (s, 4H), 2.97-2.86 (m, 3H), 2.58-2.32 (m, 2H), |
| | | | 1.47 (d, J = 8.4 Hz, 3H) |
| 293 | Similar to | m/z | ¹H NMR (400 MHz, DMSO-d6) δ = 11.00 (s, 1H), 7.81- |
| | Example | 568.5 | 7.51 (m, 2H), 7.47-7.16 (m, 6H), 6.79-6.59 (m, 2H), |
| | 22 | [M + H]⁺ | 3.98 (t, J = 7.2 Hz, 1H), 3.84 (s, 1H), 3.69 (br t, J = 7.2 Hz, |
| | | | 1H), 3.63-3.42 (m, 8H), 3.39-3.28 (m, 1H), 2.30-2.19 |
| | | | (m, 2H), 2.08-1.94 (m, 4H), 1.35 (d, J = 8.4 Hz, 3H) |
| 294 | Similar to | m/z | ¹H NMR (400 MHz, DMSO-d6) δ = 7.78-7.58 (m, 2H), |
| | Example | 582.3 | 7.44-7.32 (m, 4H), 7.29-7.20 (m, 2H), 6.82-6.72 (m, |
| | 22 | [M + H]⁺ | 2H), 4.04 (d, J = 11.6 Hz, 3H), 3.99-3.95 (m, 1H), 3.82 |
| | | | (d, J = 2.0 Hz, 1H), 3.71-3.36 (m, 10H), 2.31-2.22 (m, |
| | | | 2H), 2.10-1.94 (m, 4H), 1.37 (d, J = 7.6 Hz, 3H) |

### Example 23

### 6-((3aR,6aS)-5-(8-methyl-6-(3-methyl-3-(spiro[2.3]hexan-5-yl)pyrrolidin-1-yl)-[1,2,4]triazolo[1,5-a]pyridine-2-carbonyl)hexahydropyrrolo[3,4-c]pyrrol-2(1H)-yl)picolinic acid (Compound 23)

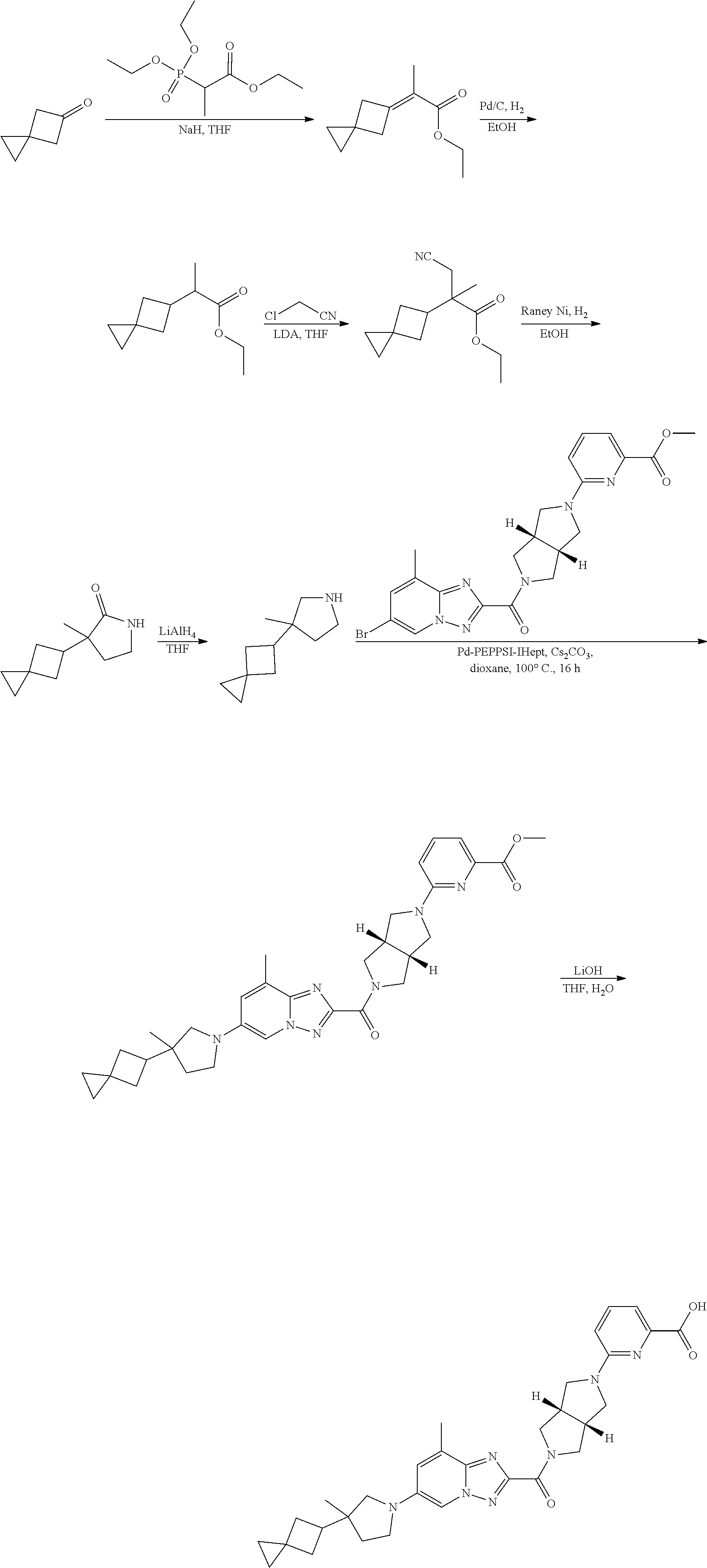

### Step 1: ethyl 2-(spiro[2.3]hexan-5-ylidene)propanoate

To a mixture of NaH (1.25 g, 31.21 mmol, 60% purity, 1.2 eq) in THF (30 mL) at 0° C. was added ethyl 2-diethoxyphosphorylpropanoate (8.67 g, 36.41 mmol, 7.93 mL, 1.4 eq) stirred at 25° C. for 0.5 h, then the mixture was added spiro[2.3]hexan-5-one (2.5 g, 26.01 mmol, 1 eq) stirred at 25° C. for 1 h. The reaction mixture was monitored by TLC (PE:EtOAc=20:1) indicated Reactant 1 (Rf=0.20) was consumed completely and one new spot (Rf=0.70) formed. The reaction mixture was quenched with a sat. aq NH₄Cl solution (10 mL) and water (10 mL), The aqueous phase was extracted with EtOAc (10 mL×3). The combined organic phases were washed with brine (30 mL), dried over Na2SO4 filtered and concentrated under reduced pressure to give crude product. The residue was purified by prep-TLC (SiO₂, PE:EtOAc=20:1, Rf=0.7) to give compound ethyl 2-spiro[2.3]hexan-5-ylidenepropanoate (4.2 g, 86% yield) as colourless liquid.

¹H NMR (400 MHz, CHLOROFORM-d) δ=4.15 (q, J=7.2 Hz, 2H), 3.13 (br d, J=2.0 Hz, 2H), 2.85 (br s, 2H), 1.72 (s, 3H), 1.26 (t, J=7.2 Hz, 3H), 0.54 (br d, J=3.2 Hz, 4H).

### Step 2: ethyl 2-(spiro[2.3]hexan-5-yl)propanoate

To a mixture of ethyl 2-spiro[2.3]hexan-5-ylidenepropanoate (2 g, 11.10 mmol, 1 eq), Pd/C (1.18 g, 1.11 mmol, 10% purity, 0.1 eq) in EtOH (20 mL), then the mixture was stirred at 25° C. for 1 h under H2 (22.37 mg, 11.10 mmol, 1 eq) (15 psi). LCMS showed starting material was consumed and one main peak with desired mass was detected. The reaction was filtered and the filtered concentrated under reduced pressure to give a residue. The residue was purified by column chromatography (SiO₂, PE:EtOAc=5:1, Rf=0.8) to give compound ethyl 2-spiro[2.3]hexan-5-ylpropanoate (1.6 g, 72% yield) as colorless liquid.

¹H NMR (400 MHz, CHLOROFORM-d) δ=4.16-4.02 (m, 2H), 2.61-2.40 (m, 2H), 2.11-1.82 (m, 4H), 1.24-1.20 (m, 3H), 1.13-1.03 (m, 3H), 0.47-0.26 (m, 4H).

### Step 3: ethyl 3-cyano-2-methyl-2-(spiro[2.3]hexan-5-yl)propanoate

To a mixture of ethyl 2-spiro[2.3]hexan-5-ylpropanoate (1.6 g, 8.78 mmol, 1 eq) in THF (15 mL) was added LDA (2.5 M, 7.02 mL, 2 eq) at −78° C. for 1 h, then the mixture was added 2-chloroacetonitrile (1.99 g, 26.34 mmol, 1.67 mL, 3 eq) at −78° C. and stirred at 25° C. for 1 h. LCMS showed starting material was consumed and one main peak with desired mass was detected. The reaction mixture was quenched by NH₄Cl (10 ml) and extracted with EtOAc and water, The aqueous phase was extracted with EtOAc (10 mL×3). The combined organic phase was washed with brine (30 mL), dried with anhydrous Na2SO4, filtered and concentrated in vacuum. The residue was purified by column chromatography (SiO₂, PE:EtOAc=5:1, Rf=0.6) to give compound ethyl 3-cyano-2-methyl-2-spiro[2.3]hexan-5-yl-propanoate (1 g, 51% yield) as colorless liquid.

¹H NMR (400 MHz, CHLOROFORM-d) δ=4.26-4.11 (m, 2H), 2.77 (t, J=8.4 Hz, 1H), 2.66 (d, J=16.4 Hz, 1H), 2.43 (d, J=16.8 Hz, 1H), 2.16-2.05 (m, 2H), 2.01-1.92 (m, 2H), 1.35-1.22 (m, 6H), 0.48-0.39 (m, 2H), 0.37-0.27 (m, 2H).

### Step 4: 3-methyl-3-(spiro[2.3]hexan-5-yl)pyrrolidin-2-one

To a mixture of ethyl 3-cyano-2-methyl-2-spiro[2.3]hexan-5-yl-propanoate (1 g, 4.52 mmol, 1 eq), RANEY NICKEL (38.72 mg, 451.89 μmol, 0.1 eq), NH₃·H₂O (910.00 mg, 7.27 mmol, 1 mL, 28% purity, 1.61 eq) in EtOH (20 mL), then the mixture was stirred at 50° C. for 12 h under H2 (9.11 mg, 4.52 mmol, 1 eq) (50 psi), LCMS showed starting material was consumed and one main peak with desired mass was detected. The reaction mixture was filtered. The residue was purified by column chromatography (SiO₂, PE:EtOAc=5:1, Rf=0.05) to give the compound 3-methyl-3-spiro[2.3]hexan-5-yl-pyrrolidin-2-one (200 mg, 18% yield) as yellow solid.

¹H NMR (400 MHz, CHLOROFORM-d) δ=6.72-6.07 (m, 1H), 3.40-3.22 (m, 2H), 2.74 (quin, J=8.4 Hz, 1H), 2.37-2.25 (m, 1H), 2.14-1.93 (m, 4H), 1.91-1.80 (m, 1H), 1.13 (s, 3H), 0.54-0.25 (m, 4H)

### Step 5: 3-methyl-3-(spiro[2.3]hexan-5-yl)pyrrolidine

To a mixture of 3-methyl-3-spiro[2.3]hexan-5-yl-pyrrolidin-2-one (150 mg, 836.78 μmol, 1 eq) in THF (2 mL) at 0° C., then the mixture was added LiAlH₄ (2.5 M, 1.00 mL, 3 eq) stirred at 70° C. for 1 h, LCMS showed starting material was consumed and one main peak with desired mass was detected. Then mixture was add appropriate amount of Potass (0.05 g) into the reaction solution to quench and filter give the compound 3-methyl-3-spiro[2.3]hexan-5-yl-pyrrolidine (130 mg, 94 yield) was obtained as colorless oil.

LC-MS [ESI, M+1]: 166.2.

### Step 6: methyl 6-((3aR,6aS)-5-(8-methyl-6-(3-methyl-3-(spiro[2.3]hexan-5-yl)pyrrolidin-1-yl)-[1,2,4]triazolo[1,5-a]pyridine-2-carbonyl)hexahydropyrrolo[3,4-c]pyrrol-2(1H)-yl)picolinate

To a mixture of methyl 6-[5-(6-bromo-8-methyl-[1,2,4]triazolo[1,5-a]pyridine-2-carbonyl)-1,3,3a, 4,6,6a-hexahydropyrrolo[3,4-c]pyrrol-2-yl]pyridine-2-carboxylate (100 mg, 206.04 μmol, 1 eq), 3-methyl-3-spiro[2.3]hexan-5-yl-pyrrolidine (40.86 mg, 247.25 μmol, 1.2 eq), Cs2CO3 (134.27 mg, 412.09 μmol, 2 eq), 1,3-bis[2,6-bis(1-propylbutyl)phenyl]-4,5-dichloro-2H-imidazol-1-ium-2-ide;3-chloropyridine;dichloropalladium (4.01 mg, 4.12 μmol, 0.02 eq) in dioxane (2 mL), then the mixture was stirred at 100° C. for 1 h, LCMS showed starting material was consumed and one main peak with desired mass was detected, The reaction mixture was extracted with EtOAc, The aqueous phase was extracted with EtOAc (10 mL×3). The combined organic phase was washed with brine (15 mL), dried with anhydrous Na2SO4, filtered and concentrated in vacuum, The residue was purified by column chromatography (SiO₂, DCM:MeOH=15:1, Rf=0.5) to give compound methyl 6-[5-[8-methyl-6-(3-methyl-3-spiro[2.3]hexan-5-yl-pyrrolidin-1-yl)-[1,2,4]triazolo[1,5-a]pyridine-2-carbonyl]-1,3,3a, 4,6,6a-hexahydropyrrolo[3,4-c]pyrrol-2-yl]pyridine-2-carboxylate (65 mg, 37% yield) as green solid.

### Step 7: 6-((3aR,6aS)-5-(8-methyl-6-(3-methyl-3-(spiro[2.3]hexan-5-yl)pyrrolidin-1-yl)-[1,2,4]triazolo[1,5-a]pyridine-2-carbonyl)hexahydropyrrolo[3,4-c]pyrrol-2(1H)-yl)picolinic acid

To a mixture of methyl 6-[5-[8-methyl-6-(3-methyl-3-spiro[2.3]hexan-5-yl-pyrrolidin-1-yl)-[1,2,4]triazolo[1,5-a]pyridine-2-carbonyl]-1,3,3a, 4,6,6a-hexahydropyrrolo[3,4-c]pyrrol-2-yl]pyridine-2-carboxylate (65 mg, 76.44 μmol, 1 eq), LiOH·H₂O (9.62 mg, 229.33 μmol, 3 eq) in THF (1.5 mL), H₂O (0.5 mL), then the mixture was stirred at 25° C. for 1 h, LCMS showed starting material was consumed and one main peak with desired mass was detected The reaction mixture was filtered, the residue was adjusted pH around 3 by FA, The residue was purified by prep-HPLC (column: CD04-Welch Ultimate C18 150*25*7 um; mobile phase: [water(FA)—ACN]; gradient:40%-70% B over 8 min) and lyophilized to give compound 6-[5-[8-methyl-6-(3-methyl-3-spiro[2.3]hexan-5-yl-pyrrolidin-1-yl)-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (23.84 mg, 56% yield) as white solid.

LC-MS [ESI, M+1]: 556.3.

¹H NMR (400 MHz, CHLOROFORM-d) δ=7.66 (t, J=8.0 Hz, 1H), 7.58 (s, 1H), 7.50 (d, J=7.2 Hz, 1H), 6.95 (s, 1H), 6.63 (d, J=8.4 Hz, 1H), 4.38 (br dd, J=7.2, 11.9 Hz, 1H), 4.13-3.99 (m, 2H), 3.88-3.74 (m, 3H), 3.60-3.46 (m, 2H), 3.44-3.30 (m, 2H), 3.27-3.12 (m, 3H), 2.94 (d, J=8.8 Hz, 1H), 2.69-2.57 (m, 4H), 2.07-1.96 (m, 5H), 1.73 (ddd, J=4.4, 7.3, 12.2 Hz, 1H), 1.13 (s, 3H), 0.52-0.43 (m, 2H), 0.42-0.31 (m, 2H).

| Cmpd. No. | Synthesis | LC/MS | ¹H NMR |
|---|---|---|---|
| 295 | Similar to | m/z 492.2 | ¹H NMR (400 MHz, CHLOROFORM-d) δ = 7.71- |
| | Example 23 | [M + H]⁺ | 7.63 (m, 1H), 7.59 (s, 1H), 7.50 (d, J = 7.1 Hz, 1H), |
| | | | 6.93 (s, 1H), 6.63 (d, J = 8.4 Hz, 1H), 4.38 (br dd, J = |
| | | | 7.1, 12.1 Hz, 1H), 4.14-3.98 (m, 2H), 3.86-3.74 |
| | | | (m, 3H), 3.61-3.49 (m, 3H), 3.37 (dt, J = 3.0, 8.6 |
| | | | Hz, 1H), 3.30 (s, 2H), 3.23-3.13 (m, 2H), 2.62 (s, |
| | | | 3H), 2.13 (br dd, J = 3.0, 7.0 Hz, 1H), 2.11-2.06 (m, |
| | | | 1H), 1.54 (s, 3H) |
| 296 | Similar to | m/z 570.4 | ¹H NMR (400 MHz, CHLOROFORM-d) δ = 7.67 |
| | Example 23 | [M + H]⁺ | (br t, J = 7.6 Hz, 1H), 7.60-7.45 (m, 2H), 6.94 (br s, |
| | | | 1H), 6.64 (br d, J = 8.4 Hz, 1H), 4.45-4.33 (m, 1H), |
| | | | 4.14-3.98 (m, 2H), 3.90-3.74 (m, 3H), 3.60-3.46 |
| | | | (m, 2H), 3.41-3.26 (m, 2H), 3.23-3.08 (m, 3H), |
| | | | 2.86 (br d, J = 8.4 Hz, 1H), 2.63 (br s, 3H), 2.32- |
| | | | 2.24 (m, 1H), 2.07-1.92 (m, 5H), 1.88-1.81 (m, |
| | | | 4H), 1.71 (br t, J = 10.8 Hz, 3H), 1.04 (s, 3H) |
| 297 | Similar to | m/z 550.3 | ¹H NMR (400 MHz, CHLOROFORM-d) δ = 7.66 (t, |
| | Example 23 | [M + H]⁺ | J = 8.0 Hz, 1H), 7.50 (d, J = 7.2 Hz, 1H), 7.43-7.38 |
| | | | (m, 2H), 7.37-7.27 (m, 4H), 7.05 (br s, 1H), 6.61 (d, |
| | | | J = 8.4 Hz, 1H), 4.30 (dd, J = 7.2, 12.1 Hz, 1H), 4.02 |
| | | | (dd, J = 7.2, 13.1 Hz, 1H), 3.93 (dd, J = 4.8, 12.3 Hz, |
| | | | 1H), 3.85-3.71 (m, 3H), 3.63 (br s, 2H), 3.48 (dt, J = |
| | | | 3.6, 11.1 Hz, 2H), 3.20-3.06 (m, 2H), 2.99 (br s, |
| | | | 1H), 2.54 (s, 3H), 2.20 (br s, 2H), 1.99 (br s, 2H) |
| 298 | Similar to | m/z 580.8 | ¹H NMR (400 MHz, DMSO-d₆) δ = 7.91 (s, 1H), |
| | Example 23 | [M + H]⁺ | 7.64 (t, J = 7.6 Hz, 1H), 7.35-7.27 (m, 5H), 7.27- |
| | | | 7.18 (m, 2H), 6.69 (d, J = 8.4 Hz, 1H), 4.09 (br dd, |
| | | | J = 6.4, 11.6 Hz, 1H), 3.97 (br d, J = 10.0 Hz, 1H), |
| | | | 3.85 (br dd, J = 6.8, 12.8 Hz, 1H), 3.81-3.60 (m, |
| | | | 4H), 3.58-3.49 (m, 3H), 3.45 (br d, J = 3.6 Hz, 1H), |
| | | | 3.10 (br d, J = 9.2 Hz, 3H), 2.54 (s, 3H), 1.39 (s, 3H), |
| | | | 1.20 (s, 3H), 0.66 (s, 3H) |
| 299 | Similar to | m/z 574.3 | ¹H NMR (400 MHz, CHLOROFORM-d) δ = 7.70- |
| | Example 23 | [M + H]⁺ | 7.59 (m, 2H), 7.49 (d, J = 7.2 Hz, 1H), 6.98 (s, 1H), |
| | | | 6.63 (d, J = 8.4 Hz, 1H), 4.37 (br dd, J = 7.2, 12.1 Hz, |
| | | | 1H), 4.14-3.97 (m, 2H), 3.88-3.72 (m, 4H), 3.56- |
| | | | 3.41 (m, 5H), 3.22-3.11 (m, 2H), 2.69-2.51 (m, |
| | | | 7H), 2.35-2.24 (m, 1H), 1.70 (s, 3H) |
| 300 | Similar to | m/z 558.4 | ¹H NMR (400 MHz, CHLOROFORM-d) δ = 7.73- |
| | Example 23 | [M + H]⁺ | 7.58 (m, 2H), 7.50 (d, J = 7.2 Hz, 1H), 6.96 (s, 1H), |
| | | | 6.63 (d, J = 8.4 Hz, 1H), 4.38 (br dd, J = 7.2, 12.2 Hz, |
| | | | 1H), 4.16-3.98 (m, 2H), 3.92 (d, J = 9.3 Hz, 1H), |
| | | | 3.88-3.75 (m, 3H), 3.59-3.46 (m, 4H), 3.41 (d, J = |
| | | | 9.3 Hz, 1H), 3.24-3.14 (m, 2H), 2.73 (td, J = 6.4, |
| | | | 12.8 Hz, 1H), 2.63 (s, 3H), 2.39 (s, 3H), 2.24 (td, J = |
| | | | 7.2, 12.8 Hz, 1H), 1.65 (s, 3H) |
| 301 | Similar to | m/z 574.1 | ¹H NMR (400 MHz, CHLOROFORM-d) δ ppm 1.62 |
| | Example 23 | [M + H]⁺ | (s, 3 H) 2.17-2.26 (m, 1 H) 2.54 (s, 3 H) 2.60 (s, 3 |
| | | | H) 2.73 (dt, J = 12.8, 6.35 Hz, 1 H) 3.20-3.32 (m, 2 |
| | | | H) 3.38 (d, J = 9.2 Hz, 1 H) 3.45-3.55 (m, 3 H) 3.60 |
| | | | (br dd, J = 10.4, 4.13 Hz, 1 H) 3.72-3.91 (m, 3 H) |
| | | | 3.98 (br dd, J = 13.6, 4.75 Hz, 1 H) 4.10 (br dd, |
| | | | J = 14.8, 3.94 Hz, 1 H) 4.31-4.45 (m, 2 H) 6.64 (d, |
| | | | J = 8.4 Hz, 1 H) 6.94 (s, 1 H) 7.52 (d, J = 7.2 Hz, 1 H) |
| | | | 7.61 (s, 1 H) 7.64-7.71 (m, 1 H) |
| 302 | Similar to | m/z 507.3 | ¹H NMR (400 MHz, CHLOROFORM-d) δ ppm 1.43 |
| | Example 23 | [M + H]⁺ | (d, J = 7.2 Hz, 1 H) 1.49 (d, J = 7.6 Hz, 3 H) 1.98-2.23 |
| | | | (m, 5 H) 2.44-2.55 (m, 1 H) 2.56-2.67 (m, 3 H) |
| | | | 3.23-3.54 (m, 5 H) 3.55-3.79 (m, 2 H) 3.74-3.75 |
| | | | (m, 1 H) 3.76-3.79 (m, 1 H) 3.82-3.92 (m, 3 H) |
| | | | 3.98-4.03 (m, 1 H) 4.13-4.27 (m, 1 H) 6.10 (dd, |
| | | | J = 7.6, 2.19 Hz, 1 H) 6.62 (t, J = 8.4 Hz, 1 H) 6.97 (br |
| | | | d, J = 9.6 Hz, 1 H) 7.28 (d, J = 2.4 Hz, 1 H) 7.48 (t, |
| | | | J = 6.8 Hz, 1 H) 7.55-7.69 (m, 2 H) |

### Example 24

### Lithium 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate (Compound 24)

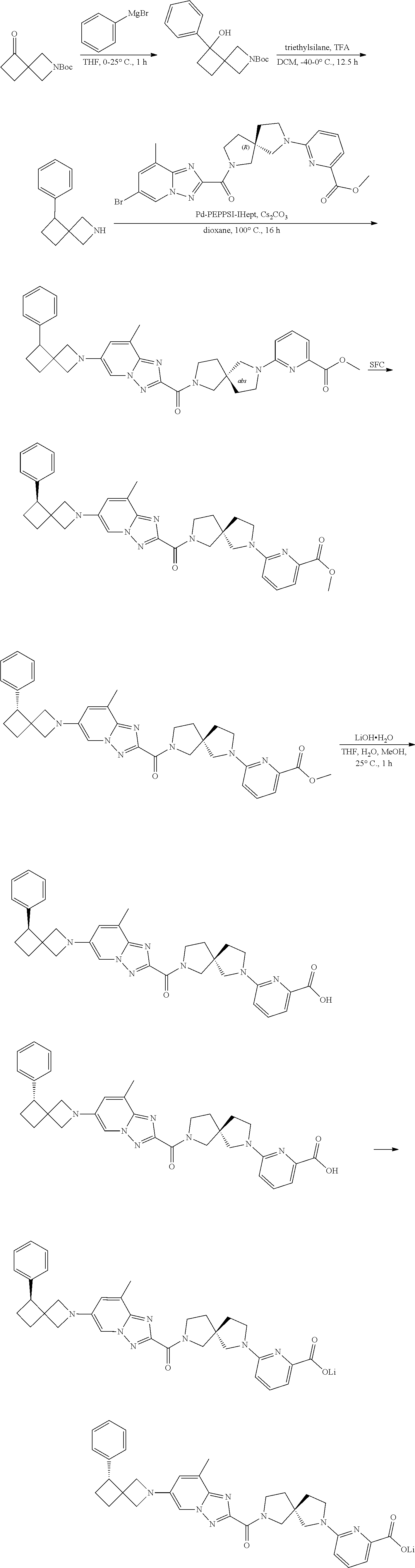

### Step 1: tert-butyl 5-hydroxy-5-phenyl-2-azaspiro[3.3]heptane-2-carboxylate

To a solution of tert-butyl 5-oxo-2-azaspiro[3.3]heptane-2-carboxylate (920 mg, 4.35 mmol) in THF (10 mL) was added phenylmagnesium bromide (3 M, 5.81 mL) at −40° C. The mixture was stirred at 0° C. for 1 hr. The reaction mixture was quenched by NH₄Cl (20 mL), extracted with Ethyl acetate (100 mL×2). The combined organic layers were washed with brine (120 mL×2), dried over Na2SO4, filtered and concentrated under reduced pressure to give a residue. The residue was purified prep-HPLC (column: Phenomenex luna C18 150*40 mm*15 um; mobile phase: [water(FA)—ACN]; gradient:42%-72% B over 15 min) to give the title compound (850 mg, 67.45% yield) as yellow solid.

¹H NMR (400 MHz, DMSO-d6) δ=7.46-7.33 (m, 4H), 7.31-7.22 (m, 1H), 5.78-5.72 (m, 1H), 4.30 (d, J=8.4 Hz, 1H), 3.62 (s, 1H), 3.44-3.36 (m, 1H), 3.15 (d, J=8.8 Hz, 1H), 2.45-2.38 (m, 1H), 2.27-2.14 (m, 1H), 2.11-1.96 (m, 2H), 1.41-1.22 (m, 9H) LC-MS [ESI, M+1]: 290.1.

### Step 2: 5-phenyl-2-azaspiro[3.3]heptane

To a solution of tert-butyl 5-hydroxy-5-phenyl-2-azaspiro[3.3]heptane-2-carboxylate (800 mg, 2.76 mmol) in DCM (9 mL) was added triethylsilane (2.08 g, 17.91 mmol, 2.86 mL) at 0° C. The mixture was stirred at 0° C. for 0.34 hr. The mixture was added dropwised TFA (3.79 g, 33.26 mmol, 2.47 mL, 12.03 eq) at 0° C. for 0.17 hr. The mixture was stirred at 40° C. for 12 hr. The reaction mixture was quenched by ice H₂O (15 mL), extracted with DCM (120 mL×2). The combined organic layers were washed with brine (150 mL×2), dried over Na2SO4, filtered and concentrated under reduced pressure to give a residue. The residue was purified by prep-HPLC (column: Phenomenex luna C18 150*25 mm*10 um; mobile phase: [water(FA)—ACN]; gradient:55%-85% B over 18 min) to give the title compound (200 mg, 41.76% yield) as yellow oil.

### Step 3: methyl 6-((5R)-7-(8-methyl-6-(5-phenyl-2-azaspiro[3.3]heptan-2-yl)-[1,2,4]triazolo[1,5-a]pyridine-2-carbonyl)-2,7-diazaspiro[4.4]nonan-2-yl)picolinate

To a mixture of methyl methyl (R)-6-(7-(6-bromo-8-methyl-[1,2,4]triazolo[1,5-a]pyridine-2-carbonyl)-2,7-diazaspiro[4.4]nonan-2-yl)picolinate (250 mg, 500.64 μmol) and 5-phenyl-2-azaspiro[3.3]heptane (86.74 mg, 500.64 mol) in dioxane (3 mL) was added Cs₂CO₃ (326.24 mg, 1.00 mmol) and 1,3-bis[2,6-bis(1-propylbutyl)phenyl]-4,5-dichloro-2H-imidazol-1-ium-2-ide;3-chloropyridine;dichloropalladium (48.70 mg, 50.06 μmol) in one portion at 25° C. under N₂. The mixture was stirred at 100° C. for 16 hr. The reaction mixture was poured into water (8 mL), extracted with Ethyl acetate (20 mL×2). The combined organic layers were washed with brine (40 mL), dried over Na2SO4, filtered and concentrated under reduced pressure to give a residue. The residue was purified by prep-HPLC (column: Phenomenex luna C18 150*40 mm*15 um; mobile phase: [water(FA)—ACN]; gradient:45%-75% B over 22 min) to give the title compound (100 mg, 33.76% yield) as yellow solid.

¹H NMR (400 MHz, METHANOL-d4) δ=7.67-7.53 (m, 2H), 7.42-7.14 (m, 6H), 6.96-6.81 (m, 1H), 6.74-6.59 (m, 1H), 5.49 (s, 1H), 4.21-4.06 (m, 1H), 4.02-3.80 (m, 7H), 3.73-3.48 (m, 8H), 2.50 (d, J=14.4 Hz, 3H), 2.41-2.00 (m, 8H) LC-MS [ESI, M+1]: 592.5.

### Step 4: methyl 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate and methyl 6-((R)-7-(8-methyl-6-((R)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate

The residue was purified by column (column: DAICEL CHIRALCEL OD (250 mm*30 mm, 10 um);mobile phase: [C02-ACN/MeOH (0.1% NH3H2O)]; B %: 45%, isocratic elution mode) to give the title compound (34 mg, 34.00% yield) as yellow solid and to give the title compound 2 (23 mg, 23.00% yield) as yellow solid.

LC-MS [ESI, M+1]: 592.4.

### Step 5: 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinic acid

To a solution of methyl methyl 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate (34 mg, 57.46 mol) in MeOH (0.2 mL), THF (0.2 mL), H₂O (0.1 mL) was added LiOH·H₂O (7.23 mg, 172.38 mol). The mixture was stirred at 25° C. for 1 hr. The reaction mixture was concentrated under reduced pressure to remove solvent. The pH was adjusted to 7. The residue was purified by prep-HPLC (column: Waters Xbridge BEH C18 150*25 mm*5 um; mobile phase: [water(NH₄HCO₃)—ACN]; gradient:20%-50% B over 10 min) to give the title compound (11 mg, 33.07% yield, 99.8% purity) as yellow solid.

¹H NMR (400 MHz, METHANOL-d4) δ=7.79-7.69 (m, 1H), 7.64-7.56 (m, 1H), 7.40-7.14 (m, 6H), 6.90-6.76 (m, 2H), 4.18-4.09 (m, 1H), 4.04-3.81 (m, 4H), 3.76-3.48 (m, 9H), 2.55-2.46 (m, 3H), 2.39-2.31 (m, 1H), 2.28-2.07 (m, 7H)

LC-MS [ESI, M+1]: 578.4.

### Step 6: lithium 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate

A solution of 6-((R)-7-(8-methyl-6-((S)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinic acid (10.4 mg, 18.00 mol) in H₂O (10 mL) was added the LiOH·H₂O (679.93 g, 16.20 mol) at 0° C. The mixture was stirred at 25° C. for 0.5 hr. The mixture was lyophilized to give the title compound (8.96 mg, 85.28% yield, 100.0% purity) as white solid.

¹H NMR (400 MHz, METHANOL-d4) δ=7.64-7.48 (m, 2H), 7.40-7.12 (m, 6H), 6.91-6.83 (m, 1H), 6.59-6.47 (m, 1H), 4.18-4.06 (m, 1H), 4.00-3.87 (m, 3H), 3.81 (t, J=7.2 Hz, 1H), 3.72-3.48 (m, 8H), 2.50 (d, J=13.4 Hz, 3H), 2.40-2.31 (m, 1H), 2.28-2.02 (m, 7H) LC-MS [ESI, M+1]: 578.3.

### Step 7: 6-((R)-7-(8-methyl-6-((R)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinic acid

To a solution of methyl methyl 6-((R)-7-(8-methyl-6-((R)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate (23 mg, 38.87 mol) in MeOH (0.2 mL), THF (0.2 mL), H₂O (0.1 mL) was added LiOH·H₂O (4.89 mg, 116.61 mol). The mixture was stirred at 25° C. for 1 hr. The reaction mixture was concentrated under reduced pressure to remove solvent. The PH was adjusted to 7. The residue was purified by prep-HPLC (column: Waters Xbridge BEH C18 150*25 mm*5 um; mobile phase: [water(NH₄HCO₃)—ACN]; gradient:20%-50% B over 10 min) to give the title compound (11 mg, 48.45% yield, 98.9% purity) as yellow solid.

¹H NMR (400 MHz, METHANOL-d4) δ=7.78-7.69 (m, 1H), 7.64-7.56 (m, 1H), 7.41-7.16 (m, 6H), 6.92-6.76 (m, 2H), 4.19-4.09 (m, 1H), 4.03-3.80 (m, 4H), 3.74-3.47 (m, 8H), 2.50 (d, J=14.0 Hz, 3H), 2.37-2.05 (m, 8H) LC-MS [ESI, M+1]: 578.3.

### Step 8: lithium 6-((R)-7-(8-methyl-6-((R)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinate

A solution of 6-((R)-7-(8-methyl-6-((R)-5-phenyl-2-azaspiro[3.3]heptan-2-yl)-[1,2,4]triazolo[1,5-a]pyridine-2-carbonyl)-2,7-diazaspiro[4.4]nonan-2-yl)picolinic acid (5.58 mg, 9.66 mol) in H₂O (10 mL) was added the LiOH·H₂O (364.81 g, 8.69 mol) at 0° C. The mixture was stirred at 25° C. for 0.5 hr. The mixture was lyophilized to give the title compound (3.94 mg, 68.99% yield, 96.0% purity) as white solid.

¹H NMR (400 MHz, METHANOL-d4) δ=7.67-7.49 (m, 2H), 7.37-7.11 (m, 6H), 6.87 (d, J=10.8 Hz, 1H), 6.64-6.46 (m, 1H), 4.16-4.07 (m, 1H), 3.99-3.88 (m, 3H), 3.82 (t, J=7.2 Hz, 1H), 3.68-3.59 (m, 5H), 3.57-3.49 (m, 3H), 2.50 (d, J=12.4 Hz, 3H), 2.39-2.31 (m, 1H), 2.28-2.18 (m, 3H), 2.16-2.02 (m, 4H) LC-MS [ESI, M+1]: 578.3.

### Example 25

### 6-((3aR,6aS)-5-(8-methyl-6-(3-methyl-3-(3-methyl-1H-1,2,4-triazol-1-yl)-2-oxopyrrolidin-1-yl)-[1,2,4]triazolo[1,5-a]pyridine-2-carbonyl)hexahydropyrrolo[3,4-c]pyrrol-2(1H)-yl)picolinic acid (Compound 25)

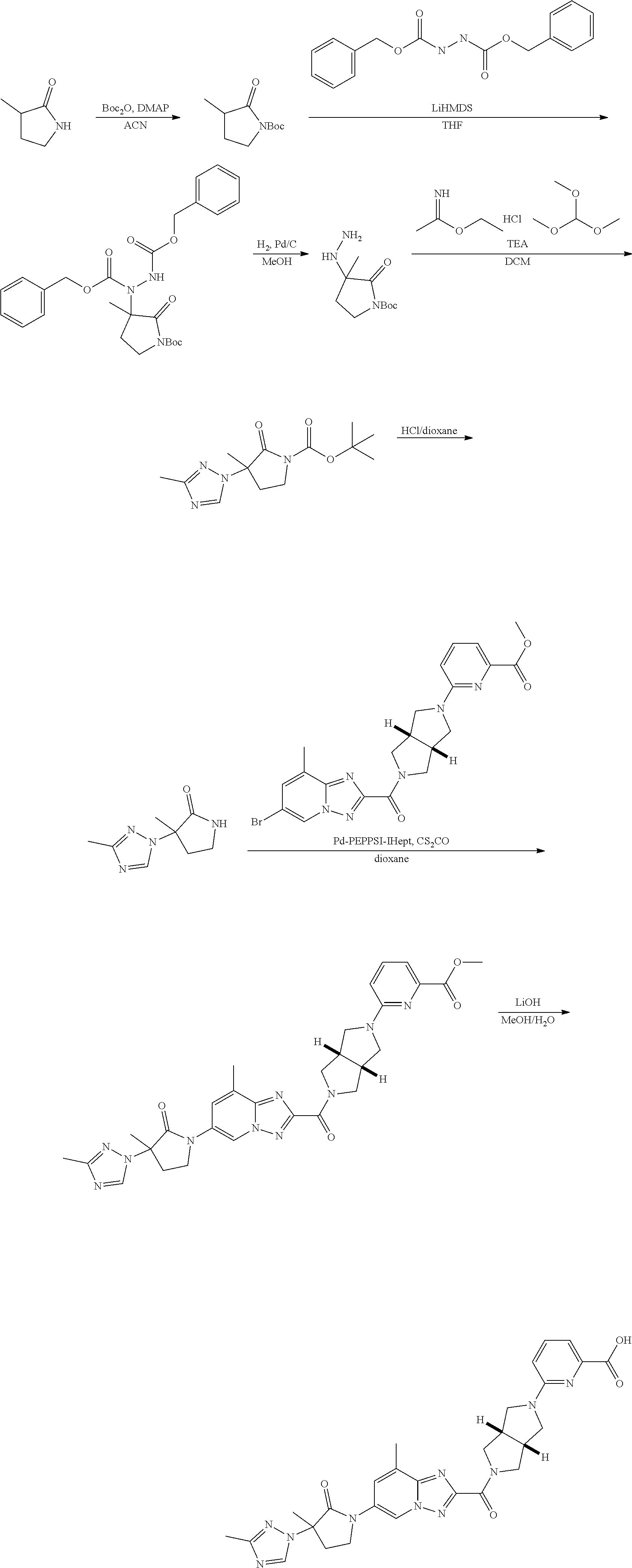

### Step 1: ethyl 4-(benzylamino)tetrahydrofuran-2-carboxylate

To a solution of 3-methylpyrrolidin-2-one (4.5 g, 45.39 mmol) in ACN (40 mL) was added the DMAP (1.11 g, 9.08 mmol) and Boc₂O (10.90 g, 49.93 mmol, 11.47 mL). The mixture was stirred at 15° C. for 12 h. The reaction mixture was concentrated under reduced pressure to give a residue. After the mixture was cooled to room temperature, the reaction mixture was diluted with H₂O (50 mL), and then extracted with ethyl acetate (30 mL×3). The combined organic layers were washed with brine (30 mL), dried over Na₂SO₄, filtered and concentrated under reduced pressure to give the title compound (9.8 g, crude) as a yellow liquid.

¹H NMR (400 MHz, CHLOROFORM-d) δ=3.74 (m, J=2.6, 8.5, 11.0 Hz, 1H), 3.55 (m, J=7.2, 9.4, 10.9 Hz, 1H), 2.60-2.47 (m, 1H), 2.18 (m, J=2.7, 7.2, 8.4, 12.6 Hz, 1H), 1.67-1.53 (m, 1H), 1.50 (s, 9H), 1.20 (d, J=7.2 Hz, 3H)

LC-MS [ESI, M+Na]: 222.1.

### Step 2: 4-(benzylamino)tetrahydrofuran-2-carboxylic acid

To a solution of tert-butyl 3-methyl-2-oxo-pyrrolidine-1-carboxylate (4.1 g, 20.58 mmol) in THF (60 mL) was added LiHMDS (1 M, 20.58 mL) dropwised (over 10 min) at −78° C. The mixture was stirred at −78° C. for 30 min. Then added the benzyl (NE)-N-benzyloxycarbonyliminocarbamate (12.28 g, 41.16 mmol) dropwised (over 10 min). The mixture was stirred at −78° C. for 16 h. The reaction mixture was quenched by NH₄Cl (200 mL), extracted with Ethyl acetate (100 mL×2). The combined organic layers were washed with brine (230 mL×2), dried over Na2SO4, filtered and concentrated under reduced pressure to give a residue. The residue was purified by reversed-phase MPLC (0.1% FA condition) and the mobile phase was lyophilized to remove solvent to give the title compound (8.1 g, 42.63% yield, 100% purity) as a brown gum.

LC-MS [ESI, M-55]: 442.1.

### Step 3: (2S,4R)-4-(benzylamino)-N-(1-(2-(methyl(2-(p-tolyloxy)ethyl)amino)-2-oxoethyl)-1H-pyrazol-4-yl)tetrahydrofuran-2-carboxamide

A solution of tert-butyl 3-[benzyloxycarbonyl(benzyloxycarbonylamino)amino]-3-methyl-2-oxo-pyrrolidine-1-carboxylate (12 g) in MeOH (240 mL) was dissolved to becoming a clear solution S1. The fixed bed (named FLR1, volume 15 mL) was packed with granular catalyst 5% Pd/Al2O3 (WXC1035, 6 g) then heated to 60° C. The H2 back pressure regulator was adjusted to 1 MPa. The above solution S1 was pumped into the fixed bed {FLR1,SS,Fixed bed, 3 mL, 60° C.} at a flow rate of {S1,P1,0.9 mL/min}, and adjusted the flow rate of H2 to 50 mL/min. The reaction mixture was flowing through {FLR1,3.3 min} to leaving the reaction zoom, then collected by container. The reaction mixture was concentrated under reduced pressure to remove solvent to give the title compound (5.8 g, 22.77 mmol, 78.12% yield, 90% purity) was obtained as a light yellow solid.

LC-MS [ESI, M-99]: 130.1.

### Step 4: (2S,4R)-4-amino-N-(1-(2-(methyl(2-(p-tolyloxy)ethyl)amino)-2-oxoethyl)-1H-pyrazol-4-yl)tetrahydrofuran-2-carboxamide

To a solution of ethyl ethanimidate (1.08 g, 8.72 mmol, HCl) in DCM (10 mL) was added TEA (882.69 mg, 8.72 mmol, 1.21 mL) at 0° C. And then, to the reaction mixture was added tert-butyl 3-hydrazino-3-methyl-2-oxo-pyrrolidine-1-carboxylate (1 g, 4.36 mmol) at 0° C., the mixture was stirred at 45° C. for 1 h. And then, the reaction mixture was concentrated under reduced pressure to remove solvent. Then to the residue was added trimethoxymethane (10 mL) and stirred at 100° C. for 5 h. The reaction mixture was poured into water (100 ml) and extracted with ethyl acetate (50 mL×2). The combined organic layer was washed with brine (70 mL×2), dried over anhydrous sodium sulfate, filtered and concentrated under reduced pressure to remove solvent to give the title compound (1.3 g, crude) as a brown gum. The residue was purified by prep-HPLC (column: Phenomenex luna C18 150*40 mm*15 um; mobile phase: [water(FA)—ACN]; gradient:22%-52% B over 15 min) and the mobile phase was lyophilized to remove solvent to give the title compound (0.23 g, 23.00% yield, 100% purity) as a brown solid.

LC-MS [ESI, M+1]: 281.2.

### Step 5: (2S,4R)-4-((2-fluoropyrimidin-4-yl)amino)-N-(1-(2-(methyl(2-(p-tolyloxy)ethyl)amino)-2-oxoethyl)-1H-pyrazol-4-yl)tetrahydrofuran-2-carboxamide

A solution of tert-butyl 3-methyl-3-(3-methyl-1,2,4-triazol-1-yl)-2-oxo-pyrrolidine-1-carboxylate (30 mg, 107.02 mol) in HCl/dioxane (2 M, 1.50 mL) was stirred at 20° C. for 16 h. The reaction mixture was concentrated under reduced pressure to remove solvent to give the title compound (23 mg, 99.19% yield, HCl) as a colourless gum.

LC-MS [ESI, M+1]: 181.1.

### Step 6: (2S,4R)-4-((2-fluoropyrimidin-4-yl)amino)-N-(1-(2-(methyl(2-(p-tolyloxy)ethyl)amino)-2-oxoethyl)-1H-pyrazol-4-yl)tetrahydrofuran-2-carboxamide

To a mixture of methyl 6-[5-(6-bromo-8-methyl-[1,2,4]triazolo[1,5-a]pyridine-2-carbonyl)-1,3,3a, 4,6,6a-hexahydropyrrolo[3,4-c]pyrrol-2-yl]pyridine-2-carboxylate (40.00 mg, 82.42 mol) and 3-methyl-3-(3-methyl-1,2,4-triazol-1-yl)pyrrolidin-2-one (23.00 mg, 106.15 μmol, HCl) in dioxane (1 mL) was added Cs₂CO₃ (80.56 mg, 247.25 mol) and 1,3-bis[2,6-bis(1-propylbutyl)phenyl]-4,5-dichloro-2H-imidazol-1-ium-2-ide;3-chloropyridine;dichloropalladium (8.02 mg, 8.24 mol) in one portion at 25° C. under N₂. The mixture was stirred at 100° C. for 16 hours. The reaction mixture was filtered and the filtrate was concentrated under reduced pressure to remove solvent. The residue was purified by prep-HPLC (column: Phenomenex Luna C18 150*30 mm*5 um; mobile phase: [water(FA)—ACN]; gradient:18%-48% B over 9 min) and the mobile phase was lyophilized to remove solvent to give the title compound (12 mg, 24.90% yield, 100% purity) as a white solid.

LC-MS [ESI, M+1]: 585.4.

### Step 7: (2S,4R)-4-((2-fluoropyrimidin-4-yl)amino)-N-(1-(2-(methyl(2-(p-tolyloxy)ethyl)amino)-2-oxoethyl)-1H-pyrazol-4-yl)tetrahydrofuran-2-carboxamide

To a mixture of methyl 6-[5-[8-methyl-6-[3-methyl-3-(3-methyl-1,2,4-triazol-1-yl)-2-oxo-pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylate (12 mg, 20.53 mol) in MeOH (0.5 mL) and H₂O (0.1 mL) was added LiOH·H₂O (1.72 mg, 41.05 mol) in one portion at 20° C. under N₂. The mixture was stirred at 20° C. for 1 hr. The reaction mixture was filtered and the filtrate was concentrated under reduced pressure to remove solvent. The residue was purified by prep-HPLC (column: Phenomenex luna C18 250*50 mm*15 um; mobile phase: [water(FA)—ACN]; gradient:2%-32% B over 9 min) and the mobile phase was lyophilized to remove solvent to give the title compound (1.00 mg, 8.20% yield, 96% purity) as a white solid.

¹H NMR (400 MHz, DMSO-d6) δ=12.46 (s, 1H), 9.19 (s, 1H), 8.64 (s, 1H), 8.08-8.00 (m, 1H), 7.71-7.57 (m, 1H), 7.24 (d, J=7.6 Hz, 1H), 6.68 (d, J=8.4 Hz, 1H), 4.14-3.99 (m, 3H), 3.92-3.83 (m, 1H), 3.77-3.61 (m, 3H), 3.60-3.52 (m, 1H), 3.47-3.35 (m, 3H), 3.10 (s, 2H), 2.89-2.80 (m, 1H), 2.59 (s, 3H), 2.25 (s, 3H), 1.84 (s, 3H)

LC-MS [ESI, M+1]: 571.4.

### Example 26

### [(3R,4S)-3,4-dihydroxypyrrolidin-1-yl]-[8-methyl-6-[(2R)-2-phenylpropoxy]-[1,2,4]triazolo[1,5-a]pyridin-2-yl]methanone (Compound 26)

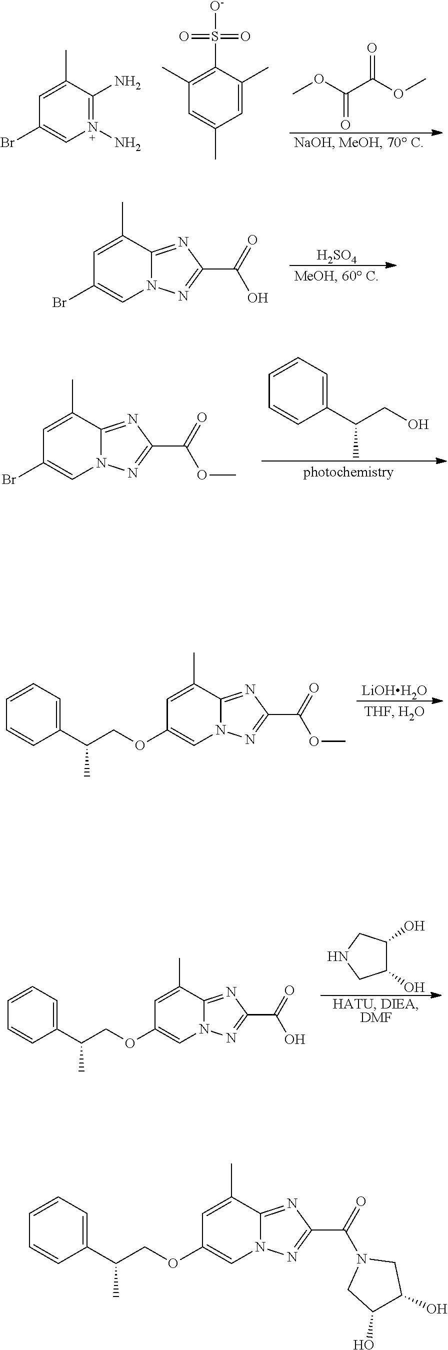

### Step 1: 6-bromo-8-methyl-[1,2,4]triazolo[1,5-a]pyridine-2-carboxylic acid

To a solution of 5-bromo-3-methyl-pyridin-1-ium-1,2-diamine;2,4,6-trimethylbenzenesulfonate (20.44 g, 50.81 mmol, 1.2 eq) and dimethyl oxalate (5 g, 42.34 mmol, 1 eq) in MeOH (100 mL) was added NaOH (5.08 g, 127.02 mmol, 3 eq) and Na2SO4 (3.01 g, 21.17 mmol, 2.15 mL, 0.5 eq). The mixture was stirred at 70° C. for 12 h. LCMS showed starting material was consumed and one main peak with desired mass was detected. The reaction mixture was filtered and the filtrate was concentrated under the reduced pressure to give Compound 6-bromo-8-methyl-[1,2,4]triazolo[1,5-a]pyridine-2-carboxylic acid (15 g, crude) was obtained as a white solid.

LC-MS [ESI, M+1]: 255.9.

### Step 2: methyl 6-bromo-8-methyl-[1,2,4]triazolo[1,5-a]pyridine-2-carboxylate

To a solution of 6-bromo-8-methyl-[1,2,4]triazolo[1,5-a]pyridine-2-carboxylic acid (15 g, 58.58 mmol, 1 eq) in MeOH (200 mL) was added H2SO4 (9.27 g, 94.54 mmol, 5.04 mL, 1.61 eq) The mixture was stirred at 60° C. for 12 h. LCMS showed starting material was consumed and one main peak with desired mass was detected. The reaction mixture was concentrated under reduced pressure to give a residue. The pH was adjusted to around 7 by progressively aq. NaHCO₃, and then added water (10 mL), extracted with EtOAc (100 mL×4). The combined organic layers were dried over Na2SO4, filtered and concentrated under reduced pressure to give a residue. The residue was purified by flash silica gel chromatography (ISCO®; 40 g SepaFlash® Silica Flash Column, Eluent of 0~30% EtOAc/PE @80 mL/min) to give Compound methyl 6-bromo-8-methyl-[1,2,4]triazolo[1,5-a]pyridine-2-carboxylate (9 g, 57% yield) was obtained as a white solid.

LC-MS [ESI, M+1]: 270.1.

### Step 3: methyl 8-methyl-6-[(2R)-2-phenylpropoxy]-[1,2,4]triazolo[1,5-a]pyridine-2-carboxylate

To a solution of methyl 6-bromo-8-methyl-[1,2,4]triazolo[1,5-a]pyridine-2-carboxylate (5 g, 18.51 mmol, 1 eq) and (2R)-2-phenylpropan-1-ol (7.56 g, 55.54 mmol, 3 eq) in ACN (2 mL) was added Ir[dF(CF₃)ppy]₂(dtbpy)(PF₆) (207.70 mg, 185.13 μmol, 0.01 eq), TMP (5.23 g, 37.03 mmol, 6.29 mL, 2 eq) and NiCl₂·dtbbpy (368.40 mg, 925.64 μmol, 0.05 eq). The mixture was stirred at 25° C. for 14 h. LCMS showed starting material was consumed and one main peak with desired mass was detected. The mixture was filtered. The residue was purified by flash silica gel chromatography (ISCO®; 20 g SepaFlash® Silica Flash Column, Eluent of 0~30% EtOAc/PE @80 mL/min) to give Compound methyl 8-methyl-6-[(2R)-2-phenylpropoxy]-[1,2,4]triazolo[1,5-a]pyridine-2-carboxylate (2.3 g, 38% yield) was obtained as a yellow solid.

LC-MS [ESI, M+1]: 326.2.

### Step 4: 8-methyl-6-[(2R)-2-phenylpropoxy]-[1,2,4]triazolo[1,5-a]pyridine-2-carboxylic acid

To a solution of methyl 8-methyl-6-[(2R)-2-phenylpropoxy]-[1,2,4]triazolo[1,5-a]pyridine-2-carboxylate (2.3 g, 7.07 mmol, 1 eq) in THF (20 mL) was added LiOH·H₂O (889.93 mg, 21.21 mmol, 3 eq) and H₂O (3 mL). The mixture was stirred at 25° C. for 2 h. LCMS showed starting material was consumed and one main peak with desired mass was detected. The reaction mixture was added HCl (1 M) until pH around 4, and then was extracted with water. The aqueous phase was extracted with EtOAc (10 mL×4), dried with anhydrous Na2SO4, filtered and concentrated in vacuum to give compound 8-methyl-6-[(2R)-2-phenylpropoxy]-[1,2,4]triazolo[1,5-a]pyridine-2-carboxylic acid (2 g, 91% yield) was obtained as a yellow solid.

LC-MS [ESI, M+1]: 312.2.

¹H NMR (400 MHz, CHLOROFORM-d) δ=8.83 (br d, J=1.6 Hz, 1H), 8.23 (d, J=1.6 Hz, 1H), 7.33-7.28 (m, 2H), 7.23 (s, 1H), 7.22-7.17 (m, 1H), 7.13 (s, 1H), 4.13-3.98 (m, 2H), 3.32-3.19 (m, 1H), 2.64 (s, 3H), 1.40 (d, J=7.2 Hz, 3H)

### Step 5: [(3R,4S)-3,4-dihydroxypyrrolidin-1-yl]-[8-methyl-6-[(2R)-2-phenylpropoxy]-[1,2,4]triazolo[1,5-a]pyridin-2-yl]methanone

To a solution of 8-methyl-6-[(2R)-2-phenylpropoxy]-[1,2,4]triazolo[1,5-a]pyridine-2-carboxylic acid (30 mg, 96.36 μmol, 1 eq) and (3S,4R)-pyrrolidine-3,4-diol (16.14 mg, 115.63 μmol, 1.2 eq, HCl) in DMF (0.5 mL) was added HATU (109.92 mg, 289.08 μmol, 3 eq) and then added DIEA (62.27 mg, 481.80 μmol, 83.92 μL, 5 eq) after 0.1 h. The mixture was stirred at 25° C. for 2 h. LCMS showed starting material was consumed and one main peak with desired mass was detected. The reaction mixture was filtered. The residue was purified by prep-HPLC (column: Welch Xtimate C18 150*25 mm*5 um; mobile phase: [water(FA)—ACN]; gradient:24%-54% B over 9 min) and lyophilized to give Compound [(3R,4S)-3,4-dihydroxypyrrolidin-1-yl]-[8-methyl-6-[(2R)-2-phenylpropoxy]-[1,2,4]triazolo[1,5-a]pyridin-2-yl]methanone (2.15 mg, 6% yield) was obtained as a brown solid.

LC-MS [ESI, M+1]: 397.1.

¹H NMR (400 MHz, DMSO-d6) δ=8.56 (d, J=2.0 Hz, 1H), 7.39-7.28 (m, 5H), 7.26-7.20 (m, 1H), 5.00 (t, J=4.8 Hz, 2H), 4.25-4.12 (m, 2H), 4.12-4.02 (m, 2H), 3.87 (dd, J=5.6, 11.4 Hz, 1H), 3.66-3.56 (m, 2H), 2.49-2.49 (m, 3H), 1.33 (d, J=7.2 Hz, 3H).

| Cmpd. No. | Synthesis | LC/MS | ¹H NMR |
|---|---|---|---|
| 303 | Similar to | m/z 437.2 | ¹H NMR (400 MHz, CHLOROFORM-d) δ = 8.00 |
| | Example 26 | [M + H]⁺ | (s, 1H), 7.82 (s, 1H), 7.41-7.33 (m, 2H), 7.30 (d, |
| | | | J = 7.2 Hz, 2H), 7.28-7.24 (m, 1H), 7.12 (s, 1H), |
| | | | 4.95-4.76 (m, 1H), 4.16-4.05 (m, 2H), 4.04-3.97 (m, |
| | | | 1H), 3.91-3.86 (m, 1H), 3.81 (dd, J = 8.4, 4.0 Hz, |
| | | | 1H), 3.73 (d, J = 10.0 Hz, 1H), 3.69-3.56 (m, 1H), |
| | | | 3.36-3.22 (m, 1H), 2.73-2.66 (m, 1H), 2.58 (s, 3H), |
| | | | 2.48-2.34 (m, 1H), 2.14-1.88 (m, 3H), 1.44 (d, J = |
| | | | 6.8 Hz, 3H). |
| 304 | Similar to | m/z 514.2 | ¹H NMR (400 MHz, CHLOROFORM-d) δ = 8.43 (s, |
| | Example 26 | [M + H]⁺ | 1H), 8.17 (d, J = 7.6 Hz, 1H), 7.95 (d, J = 1.6 Hz, 1H), |
| | | | 7.73 (br d, J = 7.4 Hz, 1H), 7.42-7.32 (m, 2H), |
| | | | 7.31-7.28 (m, 2H), 7.27-7.21 (m, 1H), 7.11 (d, J = 0.8 |
| | | | Hz, 1H), 4.82 (br d, J = 13.2 Hz, 1H), 4.37 (br d, J = |
| | | | 13.6 Hz, 1H), 4.12-4.04 (m, 1H), 4.02-3.93 (m, 1H), |
| | | | 3.29 (qd, J = 6.8, 13.7 Hz, 1H), 3.13-3.03 (m, 1H), |
| | | | 2.84-2.66 (m, 3H), 2.59 (s, 3H), 1.98-1.84 (m, 1H), |
| | | | 1.80 (br d, J = 12.4 Hz, 1H), 1.62 (br d, J = 12.4 Hz, |
| | | | 1H), 1.48-1.25 (m, 5H) |
| 305 | Similar to | m/z 501.2 | ¹H NMR (400 MHz, CHLOROFORM-d) δ = 7.98 |
| | Example 26 | [M + H]⁺ | (s, 1H), 7.73-7.64 (m, 1H), 7.56-7.48 (m, 2H), 7.42- |
| | | | 7.34 (m, 2H), 7.33-7.28 (m, 3H), 7.14 (s, 1H), 6.68 |
| | | | (d, J = 8.4 Hz, 1H), 4.95 (qd, J = 6.0, 11.6 Hz, 1H), |
| | | | 4.13-4.04 (m, 1H), 4.04-3.92 (m, 2H), 3.81-3.72 |
| | | | (m, 1H), 3.72-3.64 (m, 1H), 3.61 (br dd, J = 4.4, 10.8 |
| | | | Hz, 1H), 3.30 (sxt, J = 6.8 Hz, 1H), 2.60 (s, 3H), 2.52 |
| | | | (dt, J = 7.2, 13.6 Hz, 1H), 2.32-2.23 (m, 1H), 1.46 |
| | | | (d, J = 7.2 Hz, 3H) |
| 306 | Similar to | m/z 407.2 | ¹H NMR (CHLOROFORM-d, 400 MHz): δ = 7.94 |
| | Example 26 | [M + H]⁺ | (s, 1.0H), 7.43 (br t, J = 5.6 Hz, 1.0H), 7.32 (br d, |
| | | | J = 7.2 Hz, 2.0H), 7.20-7.29 (m, 3.0H), 7.09 (s, |
| | | | 1.0H), 4.52 (s, 1.0H), 4.01-4.07 (m, 1.1H), 3.97 (br |
| | | | d, J = 7.2 Hz, 1.1H), 3.84 (d, J = 6.4 Hz, 2.0H), 3.65 |
| | | | (s, 2.0H), 3.18-3.31 (m, 1.1H), 2.56 (s, 3.1H), 1.76 |
| | | | (br d, J = 4.4 Hz, 2.1H), 1.59 (br d, J = 4.8 Hz, 2.0H), |
| | | | 1.41 ppm (d, J = 7.2 Hz, 3.0H) |
| 307 | Similar to | m/z 515.1 | ¹H NMR (CHLOROFORM-d, 400 MHz): δ = 7.94 |
| | Example 26 | [M + H]⁺ | (s, 1.0H), 7.64 (br d, J = 7.2 Hz, 1.0H), 7.49 (br d, |
| | | | J = 7.2 Hz, 1.1H), 7.26-7.37 (m, 4.0H), 7.25 (br d, |
| | | | J = 4.4 Hz, 2.1H), 7.08 (s, 1.0H), 6.92 (br d, J = 8.4 |
| | | | Hz, 1.0H), 4.26 (br d, J = 13.2 Hz, 3.1H), 4.03 (br d, |
| | | | J = 6.4 Hz, 1.1H), 3.92-3.99 (m, 1.0H), 3.25 (br d, |
| | | | J = 6.8 Hz, 1.1H), 3.15 (br t, J = 11.9 Hz, 2.0H), 2.55 |
| | | | (s, 3.1H), 2.18 (br d, J = 11.2 Hz, 2.2H), 1.63 (br d, |
| | | | J = 9.6 Hz, 2.1H), 1.41 ppm (d, J = 7.2 Hz, 3.0H) |
| 308 | Similar to | m/z 533.2 | ¹H NMR (400 MHz, DMSO-d₆) δ = 8.60-8.48 (m, |
| | Example 26 | [M + H]⁺ | 2H), 8.02 (br d, J = 1.2 Hz, 1H), 7.43 (d, J = 2.8 Hz, |
| | | | 1H), 7.40-7.30 (m, 2H), 7.25-7.18 (m, 2H), 7.13 (br |
| | | | dd, J = 2.4, 6.8 Hz, 1H), 7.05 (dt, J = 2.4, 8.8 Hz, 1H), |
| | | | 4.28-4.12 (m, 5H), 3.31-3.27 (m, 3H), 2.51 (br s, |
| | | | 3H), 1.93 (br d, J = 10.0 Hz, 2H), 1.78-1.64 (m, 2H), |
| | | | 1.33 (d, J = 7.2 Hz, 3H) |
| 309 | Similar to | m/z 413.0 | ¹H NMR (400 MHz, CHLOROFORM-d) δ = 7.97 (br |
| | Example 26 | [M + H]⁺ | s, 1H), 7.35-7.28 (m, 1H), 7.12-6.86 (m, 4H), 4.23- |
| | | | 4.09 (m, 1H), 4.08-3.96 (m, 3H), 3.95-3.88 (m, |
| | | | 1H), 3.88-3.80 (m, 1H), 3.69-3.55 (m, 1H), 3.35- |
| | | | 3.21 (m, 1H), 2.60 (br d, J = 5.6 Hz, 3H), 2.10-1.88 |
| | | | (m, 2H), 1.54-1.38 (m, 6H) |
| 310 | Similar to | m/z 548.8 | ¹H NMR (400 MHz, DMSO-d₆) δ = 8.68 (d, J = 8.0 |
| | Example 26 | [M + H]⁺ | Hz, 1H), 8.50 (d, J = 1.2 Hz, 1H), 7.40-7.32 (m, 1H), |
| | | | 7.30 (s, 1H), 7.24-7.16 (m, 2H), 7.04 (dt, J = 2.4, 8.4 |
| | | | Hz, 1H), 4.35-4.26 (m, 1H), 4.22-4.11 (m, 2H), |
| | | | 3.28 (br d, J = 6.8 Hz, 1H), 2.92-2.83 (m, 3H), 2.49 |
| | | | (br s, 3H), 2.35-2.27 (m, 1H), 2.23 (br dd, J = 3.6, |
| | | | 6.8 Hz, 2H), 2.20-2.06 (m, 4H), 1.96-1.88 (m, 1H), |
| | | | 1.88-1.74 (m, 4H), 1.71 (br s, 1H), 1.31 (d, J = 7.2 |
| | | | Hz, 3H) |
| 311 | Similar to | m/z 541.2 | ¹H NMR (400 MHz, CHLOROFORM-d) δ = 8.00- |
| | Example 26 | [M + H]⁺ | 7.92 (m, 1H), 7.70-7.61 (m, 1H), 7.48 (t, J = 6.8 Hz, |
| | | | 1H), 7.39-7.32 (m, 2H), 7.32-7.28 (m, 2H), 7.24 (br |
| | | | s, 1H), 7.11 (br d, J = 8.8 Hz, 1H), 6.62 (t, J = 9.2 Hz, |
| | | | 1H), 4.24-4.13 (m, 1H), 4.12-4.03 (m, 1H), 4.02 (s, |
| | | | 2H), 3.91 (br t, J = 7.2 Hz, 1H), 3.86-3.74 (m, 1H), |
| | | | 3.67-3.57 (m, 2H), 3.57-3.46 (m, 2H), 3.29 (td, J = |
| | | | 7.2, 13.9 Hz, 1H), 2.60 (d, J = 10.8 Hz, 3H), 2.25- |
| | | | 2.06 (m, 4H), 1.44 (t, J = 7.2 Hz, 3H) |
| 312 | Similar to | m/z 312.2 | ¹H NMR (400 MHz, CHLOROFORM-d) δ = 8.26 (br |
| | Example 26 | [M + H]+ | s, 1H), 7.33-7.24 (m, 4H), 7.23-7.19 (m, 1H), 7.12 |
| | | | (s, 1H), 4.13-3.98 (m, 2H), 3.30-3.20 (m, 1H), 2.63 |
| | | | (s, 3H), 1.40 (d, J = 7.2 Hz, 3H) |
| 313 | Similar to | m/z | ¹H NMR (400 MHz, CHLOROFORM-d) δ = 7.98 (s, |
| | Example 26 | 421.1 | 1H), 7.44 (br d, J = 7.6 Hz, 1H), 7.39-7.32 (m, 2H), |
| | | [M + H]+ | 7.30 (d, J = 7.2 Hz, 2H), 7.27 (s, 1H), 7.13 (s, 1H), |
| | | | 4.67-4.49 (m, 1H), 4.23 (quin, J = 7.2 Hz, 1H), |
| | | | 4.12-4.04 (m, 1H), 4.04-3.96 (m, 1H), 3.37-3.21 (m, |
| | | | 1H), 2.65-2.56 (m, 3H), 2.56-2.41 (m, 3H), 2.34 |
| | | | (td, J = 6.0, 11.6 Hz, 1H), 2.09 (td, J = 10.0, 16.3 Hz, |
| | | | 2H), 2.02-1.95 (m, 3H), 1.44 (d, J = 7.2 Hz, 3H) |
| 314 | Similar to | m/z | ¹H NMR (400 MHz, CHLOROFORM-d) δ = 7.96 (s, |
| | Example 26 | 530.1 | 1H), 7.48 (br d, J = 8.0 Hz, 1H), 7.39-7.31 (m, 2H), |
| | | [M + H]+ | 7.29 (br d, J = 7.2 Hz, 2H), 7.25 (br s, 1H), 7.10 (s, |
| | | | 1H), 4.58-4.50 (m, 1H), 4.09-4.03 (m, 1H), 4.02- |
| | | | 3.94 (m, 1H), 3.43 (br d, J = 10.0 Hz, 2H), 3.34-3.19 |
| | | | (m, 2H), 2.76 (br t, J = 8.8 Hz, 2H), 2.58 (s, 4H), 2.44 |
| | | | (br d, J = 4.4 Hz, 1H), 2.28 (br d, J = 5.6 Hz, 3H), |
| | | | 2.16-2.01 (m, 6H), 1.43 (d, J = 7.2 Hz, 3H) |
| 315 | Similar to | m/z | ¹H NMR (400 MHz, CHLOROFORM-d) δ = 7.98 (br |
| | Example 26 | 409.1 | d, J = 8.4 Hz, 1H), 7.93 (s, 1H), 7.38-7.29 (m, 2H), |
| | | [M + H]+ | 7.27 (s, 3H), 7.05 (s, 1H), 4.68-4.51 (m, 1H), 4.07- |
| | | | 3.99 (m, 1H), 3.99-3.90 (m, 1H), 3.30-3.19 (m, 1H), |
| | | | 2.54 (s, 3H), 2.26-2.19 (m, 1H), 2.07-1.98 (m, 2H), |
| | | | 1.87 (br s, 2H), 1.70-1.58 (m, 1H), 1.46-1.36 (m, |
| | | | 6H) |
| 316 | Similar to | m/z 395.1 | ¹H NMR (400 MHz, CHLOROFORM-d) δ ppm 1.40-1.56 |
| | Example 26 | [M + H]⁺ | (m, 6 H) 1.86-1.96 (m, 1 H) 1.98-2.07 (m, 1 H) 2.59 (s, |
| | | | 3 H) 3.29 (sxt, J = 6.8 Hz, 1 H) 3.61 (br d, |
| | | | J = 13.2 Hz, 1 H) 3.79-3.95 (m, 2 H) 3.95-4.03 (m, |
| | | | 1 H) 4.03-4.25 (m, 2 H) 7.08 (br s, 1 H) 7.27-7.32 |
| | | | (m, 3 H) 7.32-7.40 (m, 2 H) 7.97 (br s, 1 H) |
| 317 | Similar to | m/z 383.1 | ¹H NMR (400 MHz, CHLOROFORM-d) δ = 8 (d, J = |
| | Example 26 | [M + H]+ | 2 Hz, 1H), 7.76 (br t, J = 6.0 Hz, 1H), 7.42-7.34 |
| | | | (m, 2H), 7.33-7.25 (m, 3H), 7.12 (s, 1H), 4.13- |
| | | | 4.05 (m, 1H), 4.05-3.97 (m, 1H), 3.55 (d, J = 6 Hz, |
| | | | 2H), 3.30 (sxt, J = 6.8 Hz, 1H), 2.60 (s, 3H), |
| | | | 1.45 (d, J = 6.8 Hz, 3H), 1.33 (s, 6H) |
| 318 | Similar to | m/z 381 | ¹H NMR (400 MHz, CHLOROFORM-d) δ = 8.00- |
| | Example 26 | [M + H]+ | 7.86 (m, 2H), 7.39-7.33 (m, 2H), 7.30 (br d, J = 7.2 |
| | | | Hz, 2H), 7.27-7.24 (m, 1H), 7.07 (br s, 1H), 4.63 |
| | | | (br s, 2H), 4.12-3.93 (m, 2H), 3.32-3.22 (m, 1H), |
| | | | 2.56 (br s, 3H), 2.30-2.20 (m, 2H), 2.07-1.98 (m, |
| | | | 2H), 1.44 (d, J = 6.9 Hz, 3H) |
| 319 | Similar to | m/z 381 | ¹H NMR (400 MHz, CHLOROFORM-d) δ = 8. (br |
| | Example 26 | [M + H]+ | s, 1H), 7.63 (br s, 1H), 7.39-7.33 (m, 2H), 7.32- |
| | | | 7.28 (m, 2H), 7.25 (br s, 1H), 7.10 (s, 1H), 4.30- |
| | | | 3.94 (m, 4H), 3.34-3.21 (m, 1H), 2.56 (s, 3H), 2.24- |
| | | | 2.14 (m, 2H), 1.83-1.76 (m, 1H), 1.62-1.50 (m, |
| | | | 1H), 1.44 (d, J = 6.9 Hz, 3H) |
| 320 | Similar to | m/z 515.2 | ¹H NMR (400 MHz, DMSO-d₆) δ = 8.58 (br d, J = |
| | Example 26 | [M + H]+ | 8.0 Hz, 1H), 8.51 (s, 1H), 8.01 (br d, J = 6.8 Hz, 1H), |
| | | | 7.43 (br s, 1H), 7.38-7.28 (m, 5H), 7.25-7.20 (m, |
| | | | 1H), 7.14 (br d, J = 6 Hz, 1H), 4.26-4.17 (m, 4H), |
| | | | 4.16-4.10 (m, 1H), 3.31-3.22 (m, 3H), 2.50 (br s, |
| | | | 3H), 1.93 (br d, J = 11.6 Hz, 2H), 1.77-1.63 (m, 2H), |
| | | | 1.33 (br d, J = 6.8 Hz, 3H) |
| 321 | Similar to | m/z 501.1 | ¹H NMR (400 MHz, CHLOROFORM-d) δ = 8.53- |
| | Example 26 | [M + H]+ | 8.02 (m, 2H), 7.97-7.80 (m, 1H), 7.29 (br d, J = 7.2 |
| | | | Hz, 2H), 7.26-7.09 (m, 3H), 7.02-6.85 (m, 1H), |
| | | | 6.65-6.32 (m, 1H), 4.92-4.67 (m, 1H), 4.10-3.88 |
| | | | (m, 2H), 3.83-3.43 (m, 4H), 3.21 (br d, J = 6 Hz, |
| | | | 2H), 2.41 (br s, 4H), 2.27 (br d, J = 4.4 Hz, 1H), 1.38 |
| | | | (br d, J = 6.4 Hz, 3H) |
| 322 | Similar to | m/z 392.1 | |
| | Example 26 | [M + H]⁺ | |

### Example 27

### N-(azetidin-3-yl)-8-methyl-6-[(3R)-3-methyl-3-phenyl-pyrrolidin-1-yl]-[1,2,4]triazolo[1,5-a]pyridine-2-sulfonamide (Compound 27)

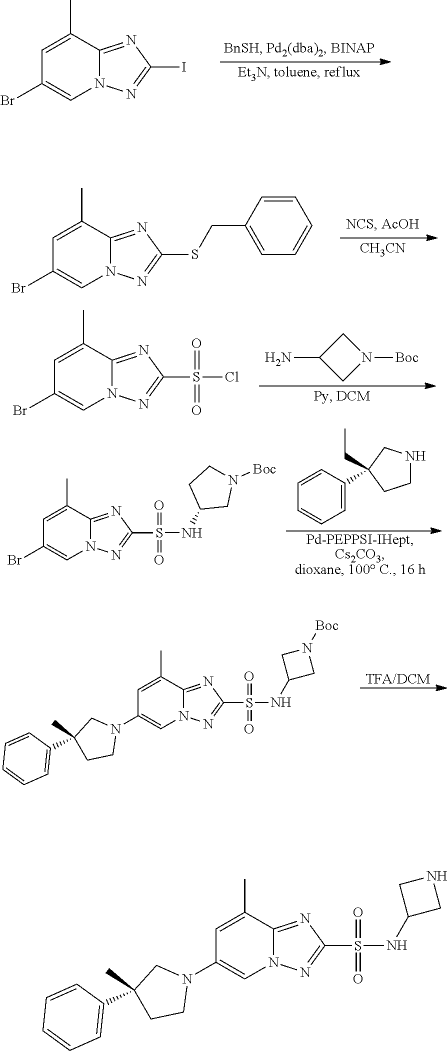

### Step 1: 2-benzylsulfanyl-6-bromo-8-methyl-[1,2,4]triazolo[1,5-a]pyridine

To a solution of 6-bromo-2-iodo-8-methyl-[1,2,4]triazolo[1,5-a]pyridine (800 mg, 2.37 mmol, 1 eq) and phenylmethanethiol (264.62 mg, 2.13 mmol, 250.11 μL, 0.9 eq) in toluene (10 mL) was added Pd₂(dba)₃ (216.77 mg, 236.73 μmol, 0.1 eq), TEA (718.62 mg, 7.10 mmol, 988.48 μL, 3 eq) and BINAP (294.81 mg, 473.45 μmol, 0.2 eq) was degassed and purged with N₂ for 3 times. The mixture was stirred at 90° C. for 2 h under N₂. LCMS showed starting material was consumed and one main peak with desired mass was detected. The reaction mixture was partitioned between H₂O 5 mL and EtOAc 30 mL (10 mL×3). The organic phase was separated, washed with brine 30 mL (10 mL×3), dried over Na2SO4, filtered and concentrated under reduced pressure to give a residue. The residue was purified by flash silica gel chromatography (ISCO®; 20 g SepaFlash® Silica Flash Column, Eluent of 0~5% EtOAc/PEt @80 mL/min) to give Compound 2-benzylsulfanyl-6-bromo-8-methyl-[1,2,4]triazolo[1,5-a]pyridine (710 mg, 90% yield) was obtained as a yellow gum

LC-MS [ESI, M+1]: 334.1.

¹H NMR (400 MHz, CHLOROFORM-d) δ=8.32 (s, 1H), 7.60 (d, J=16.0 Hz, 1H), 7.32 (d, J=7.2 Hz, 2H), 7.27 (s, 3H), 4.35 (s, 2H), 2.45 (s, 3H)

### Step 2: 6-bromo-8-methyl-[1,2,4]triazolo[1,5-a]pyridine-2-sulfonyl chloride

To a solution of 2-benzylsulfanyl-6-bromo-8-methyl-[1,2,4]triazolo[1,5-a]pyridine (200 mg, 598.38 μmol, 1 eq) in ACN (2 mL) was added NCS (239.71 mg, 1.80 mmol, 3 eq), AcOH (0.2 mL) and H₂O (0.02 mL). The mixture was stirred at 25° C. for 12 h. TLC (PE:EtOAc=3:1, Rf=0.4) indicated reactant was consumed completely and one new spot was formed. The reaction was added water (5 mL) and then extracted with DCM (5 mL×3). The combined organic phase was washed with brine (5 mL×3), dried over anhydrous Na2SO4, filtered and concentrated in vacuo to give compound 6-bromo-8-methyl-[1,2,4]triazolo[1,5-a]pyridine-2-sulfonyl chloride (200 mg, crude) was obtained as a yellow gum.

### Step 3: tert-butyl 3-((6-bromo-8-methyl-[1,2,4]triazolo[1,5-a]pyridine)-2-sulfonamido)azetidine-1-carboxylate

To a solution of 6-bromo-8-methyl-[1,2,4]triazolo[1,5-a]pyridine-2-sulfonyl chloride (100 mg, 322.00 μmol, 1 eq) and tert-butyl 3-aminoazetidine-1-carboxylate (55.46 mg, 322.00 μmol, 1 eq) in DMF (1 mL) was added DIEA (124.85 mg, 966.01 μmol, 168.26 μL, 3 eq) and tert-butyl 3-aminoazetidine-1-carboxylate (55.46 mg, 322.00 μmol, 1 eq). The mixture was stirred at 30° C. for 1 h. LCMS showed starting material was consumed and one main peak with desired mass was detected. The reaction mixture was extracted with water, the aqueous phase was extracted with EtOAc (10 mL×3). The combined organic phase was washed with brine (30 mL), dried with anhydrous Na2SO4, filtered and concentrated in vacuum. The residue was purified by column chromatography (SiO₂, PE:EtOAc=3:1) to give Compound tert-butyl 3-((6-bromo-8-methyl-[1,2,4]triazolo[1,5-a]pyridine)-2-sulfonamido)azetidine-1-carboxylate (60 mg, 42% yield) was obtained as a yellow solid.

LC-MS [ESI, M+1]: 326.2.

¹H NMR (400 MHz, CHLOROFORM-d) δ=8.63 (s, 1H), 7.56 (s, 1H), 4.21 (br t, J=8.6 Hz, 2H), 4.18-4.08 (m, 2H), 3.71-3.60 (m, 1H), 2.67 (s, 3H), 1.42 (s, 9H).

### Step 4: tert-butyl 3-[[8-methyl-6-[(3R)-3-methyl-3-phenyl-pyrrolidin-1-yl]-[1,2,4]triazolo[1,5-a]pyridin-2-yl]sulfonylamino]azetidine-1-carboxylate

To a solution of tert-butyl 3-[(6-bromo-8-methyl-[1,2,4]triazolo[1,5-a]pyridin-2-yl)sulfonylamino]azetidine-1-carboxylate (50 mg, 112.03 μmol, 1 eq) and (3R)-3-methyl-3-phenyl-pyrrolidine (18.06 mg, 112.03 μmol, 1 eq) in dioxane (0.5 mL) was added Pd-PEPPSI-IHeptCl (1.09 mg, 1.12 μmol, 0.01 eq) and Cs₂CO₃ (109.50 mg, 336.08 μmol, 3 eq). The mixture was stirred at 100° C. for 12 h under N₂. LCMS showed starting material was consumed and one main peak with desired mass was detected. The reaction mixture was filtered. The residue was purified by column chromatography (SiO₂, PE:EtOAc=0:1) to give Compound tert-butyl 3-[[8-methyl-6-[(3R)-3-methyl-3-phenyl-pyrrolidin-1-yl]-[1,2,4]triazolo[1,5-a]pyridin-2-yl]sulfonylamino]azetidine-1-carboxylate (15 mg, 25% yield) was obtained as a yellow solid.

LC-MS [ESI, M+1]: 527.3.

### Step 5: N-(azetidin-3-yl)-8-methyl-6-[(3R)-3-methyl-3-phenyl-pyrrolidin-1-yl]-[1,2,4]triazolo[1,5-a]pyridine-2-sulfonamide

To a solution of tert-butyl 3-[[8-methyl-6-[(3R)-3-methyl-3-phenyl-pyrrolidin-1-yl]-[1,2,4]triazolo[1,5-a]pyridin-2-yl]sulfonylamino]azetidine-1-carboxylate (15 mg, 28.48 μmol, 1 eq) in DCM (1 mL) was added TFA (307.00 mg, 2.69 mmol, 0.2 mL, 94.53 eq). The mixture was stirred at 25° C. for 2 h. LCMS showed starting material was consumed and one main peak with desired mass was detected. The reaction mixture was filtered. The residue was purified by prep-HPLC (column: Phenomenex luna C18 150*25 mm*10 um; mobile phase: [water(FA)—ACN]; gradient:18%-48% B over 10 min) and lyophilized to give Compound N-(azetidin-3-yl)-8-methyl-6-[(3R)-3-methyl-3-phenyl-pyrrolidin-1-yl]-[1,2,4]triazolo[1,5-a]pyridine-2-sulfonamide (9.54 mg, 79% yield) was obtained as a yellow solid.

LC-MS [ESI, M+1]: 427.2.

¹H NMR (400 MHz, CHLOROFORM-d) δ=8.53 (br s, 1H), 7.63 (br s, 1H), 7.39-7.28 (m, 2H), 7.27-7.11 (m, 3H), 6.94 (br s, 1H), 4.74 (br d, J=2.0 Hz, 1H), 4.58-4.02 (m, 4H), 3.60-3.37 (m, 3H), 3.32 (br s, 1H), 2.49 (br s, 3H), 2.35-2.12 (m, 2H), 1.37 (s, 3H).

| Cmpd. No. | Synthesis | LC/MS | ¹H NMR |
|---|---|---|---|
| 323 | Similar to | m/z 444.2 | ¹H NMR (400 MHz, CHLOROFORM-d) δ = 7.66 (d, |
| | Example 27 | [M + H]+ | J = 1.2 Hz, 1H), 7.41-7.35 (m, 2H), 7.33-7.28 (m, |
| | | | 2H), 7.27 (s, 1H), 7.06 (s, 1H), 5.56-5.42 (m, 1H), |
| | | | 3.62 (d, J = 8.8 Hz, 1H), 3.57-3.46 (m, 2H), 3.41 (dt, |
| | | | J = 3.6, 8.7 Hz, 1H), 3.26 (d, J = 5.6 Hz, 2H), 2.83 (br |
| | | | s, 1H), 2.64 (s, 3H), 2.40 (td, J = 8.4, 12.1 Hz, 1H), |
| | | | 2.33-2.23 (m, 1H), 1.47 (s, 3H), 1.28 (s, 6H) |
| 324 | Similar to | m/z 386.2 | ¹H NMR (400 MHz, DMSO-d6) δ = 8.00 (s, 2H), 7.45- |
| | Example 27 | [M + H]+ | 7.29 (m, 5H), 7.27-7.20 (m, 1H), 3.64-3.58 (m, |
| | | | 1H), 3.57-3.46 (m, 2H), 3.43-3.36 (m, 1H), 2.64 (s, |
| | | | 3H), 2.54 (s, 3H), 2.27 (br t, J = 6.8 Hz, 2H), 1.36 (s, |
| | | | 3H) |
| 325 | Similar to | m/z 455.2 | ¹H NMR (400 MHz, CHLOROFORM-d) δ = 8.55 (br |
| | Example 27 | [M + H]+ | d, J = 1.2 Hz, 1H), 7.67 (s, 1H), 7.40-7.33 (m, 2H), |
| | | | 7.33-7.28 (m, 2H), 7.27-7.22 (m, 1H), 7.03 (s, 1H), |
| | | | 3.69 (br s, 1H), 3.60 (br d, J = 8.8 Hz, 1H), 3.54-3.47 |
| | | | (m, 2H), 3.41 (dt, J = 3.2, 8.6 Hz, 1H), 3.28 (br s, 2H), |
| | | | 2.82 (br s, 2H), 2.61 (s, 3H), 2.43-2.32 (m, 1H), |
| | | | 2.31-2.21 (m, 1H), 2.04 (br s, 2H), 1.88-1.65 (m, 2H), |
| | | | 1.45 (s, 3H) |
| 326 | Similar to | m/z 441.0 | ¹H NMR (400 MHz, DMSO-d₆): δ 7.99 (d, J = 1.6 Hz, |
| | Example 27 | [M + H]⁺ | 1H), 7.43-7.31 (m, 6H), 7.26-7.21 (m, 1H), 3.81- |
| | | | 3.74 (m, 1H), 3.63-3.59 (m, 1H), 3.54-3.48 (m, 2H), |
| | | | 3.38 (br dd, J = 6.4, 9.6 Hz, 2H), 3.25-3.08 (m, 1H), |
| | | | 2.80-2.70 (m, 2H), 2.62 (ddd, J = 6.0, 8.4, 10.8 Hz, |
| | | | 1H), 2.54 (s, 3H), 2.26 (t, J = 6.8 Hz, 2H), 1.82-1.72 |
| | | | (m, 1H), 1.54-1.44 (m, 1H), 1.35 (s, 3H). |

### Example 28

### 6-[(3R)-3-(difluoromethyl)-3-phenyl-pyrrolidin-1-yl]-N-(2-hydroxy-2-methyl-propyl)-8-methyl-[1,2,4]triazolo[1,5-a]pyridine-2-carboxamide (Compound 28)

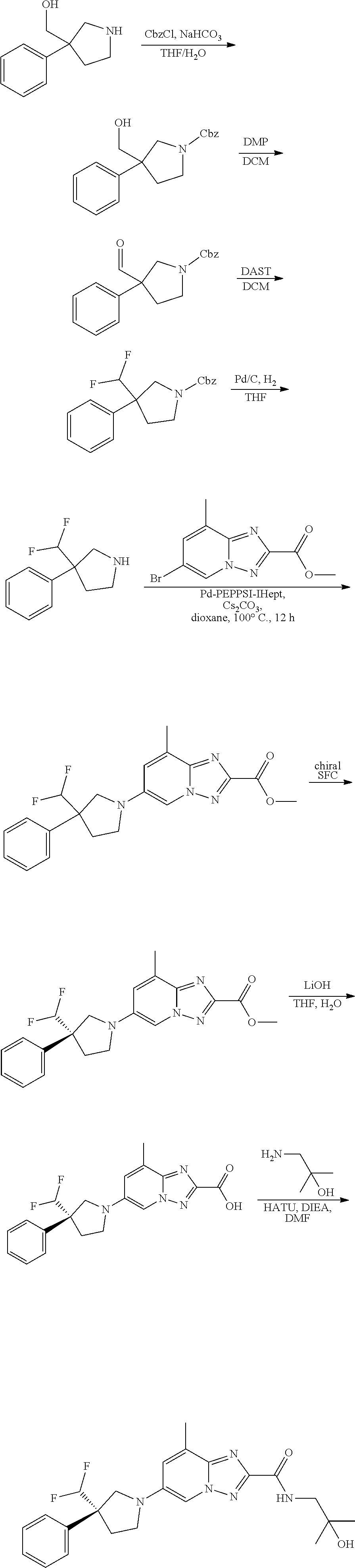

### Step 1: benzyl 3-(hydroxymethyl)-3-phenylpyrrolidine-1l-carboxylate

To a solution of (3-phenylpyrrolidin-3-yl) methanol (8.5 g, 47.96 mmol, 1 eq) in THF (60 mL) was added benzyl carbonochloridate (9.82 g, 57.55 mmol, 8.22 mL, 1.2 eq) and NaHCO₃ (20.14 g, 239.78 mmol, 9.33 mL, 5 eq) in H₂O (10 mL). The mixture was stirred at 25° C. for 1 h. TLC (PE:EtOAc=1:1, Rf=0.43) indicated reactant was consumed completely and one new spot was formed. The reaction mixture was quenched by H₂O (30 mL) at 0° C., then extracted with EtOAc (20 mL×3), the combined organic layers dried over anhydrous sodium sulfate, filtered and the filtrate was concentrated under reduced pressure to give a residue. The residue was purified by flash silica gel chromatography (ISCO®; 80 g SepaFlash® Silica Flash Column, Eluent of 0~15% EtOAc/PE gradient) to give compound benzyl 3-(hydroxymethyl)-3-phenylpyrrolidine-1-carboxylate (11 g, 73% yield) as pink oil.

LC-MS [ESI, M+1]: 312.1.

¹H NMR (400 MHz, CHLOROFORM-d) δ=7.46-7.27 (m, 8H), 7.26-7.18 (m, 2H), 5.28-5.04 (m, 2H), 3.85-3.38 (m, 6H), 2.53-2.31 (m, 1H), 2.22-2.09 (m, 1H).

### Step 2: benzyl 3-formyl-3-phenylpyrrolidine-1-carboxylate

To a solution of SO₃·Py (4.67 g, 29.32 mmol, 5.5 eq) in DMSO (15 mL) was added dropwise TEA (2.70 g, 26.66 mmol, 3.71 m L, 5 eq) at 25° C. After addition, the mixture was stirred at this temperature, and then benzyl 3-(hydroxymethyl)-3-phenyl-pyrrolidine-1-carboxylate (1.66 g, 5.33 mmol, 1 eq) in DMSO (15 mL) was added dropwise at 0° C. The resulting mixture was stirred at 80° C. for 2 h. TLC (PE:EtOAc=1:1, Rf=0.48) indicated reactant was consumed and one new spot formed. The mixture was quenched with H₂O (50 mL) and extracted with EtOAc (20 mL×3), the organic phase was dried, filtered, and concentrated under pressure to give the product. The residue was purified by flash silica gel chromatography (ISCO®; 40 g SepaFlash® Silica Flash Column, Eluent of 0~22% EtOAc/PE gradient to give compound benzyl 3-formyl-3-phenylpyrrolidine-1-carboxylate (1.35 g, 82% yield) as a yellow oil.

LC-MS [ESI, M+1]: 310.2.

¹H NMR (400 MHz, CHLOROFORM-d) δ=9.46 (d, J=5.6 Hz, 1H), 7.47-7.30 (m, 7H), 7.20 (br t, J=7.6 Hz, 2H), 5.25-5.04 (m, 2H), 4.45 (br t, J=12.4 Hz, 1H), 3.78-3.51 (m, 2H), 3.39 (br dd, J=7.2, 10.0 Hz, 1H), 2.96-2.66 (m, 1H), 2.22 (br d, J=10.0 Hz, 1H).

### Step 3: benzyl 3-(difluoromethyl)-3-phenylpyrrolidine-1-carboxylate

To a solution of DAST (2.79 g, 17.33 mmol, 2.29 mL, 4 eq) in DCM (10 mL) was added benzyl 3-formyl-3-phenylpyrrolidine-1-carboxylate (1.34 g, 4.33 mmol, 1 eq) at 0° C. The mixture was stirred at 0° C. for 1 h. LCMS showed starting material was consumed and the desired mass was detected. The mixture was quenched with H₂O (20 mL) and extracted with EtOAc (20 mL×3), the organic phase was dried, filtered, and concentrated under pressure to give the product. The residue was purified by flash silica gel chromatography (ISCO®; 40 g SepaFlash® Silica Flash Column, Eluent of 0~24% EtOAc/PE gradient) to give compound benzyl 3-(difluoromethyl)-3-phenylpyrrolidine-1-carboxylate (0.8 g, 56% yield) as a yellow oil.

LC-MS [ESI, M+1]: 332.0.

¹H NMR (400 MHz, CHLOROFORM-d) δ=7.43-7.33 (m, 10H), 5.82 (t, J=56.4 Hz, 1H), 5.20-5.13 (m, 2H), 3.85-3.64 (m, 2H), 3.63-3.52 (m, 2H), 2.63 (br dd, J=5.2, 12.4 Hz, 1H), 2.33 (br dd, J=8.8, 12.4 Hz, 1H).

### Step 4: 3-(difluoromethyl)-3-phenylpyrrolidine

To a solution of benzyl 3-(difluoromethyl)-3-phenyl-pyrrolidine-1-carboxylate (0.8 g, 2.41 mmol, 1 eq) in THF (10 mL) was added Pd/C (513.86 mg, 482.86 μmol, 10% purity, 0.2 eq) and Pd(OH)₂ (678.11 mg, 482.86 μmol, 10% purity, 0.2 eq). The suspension was degassed and purged with H2 for 3 times. The mixture was stirred under H2 (50 Psi) at 70° C. for 12 h. LCMS showed starting material was consumed and the desired mass was detected. The reaction was concentrated under reduced pressure to give compound 3-(difluoromethyl)-3-phenylpyrrolidine (0.55 g, 97% yield) as a pink oil.

LC-MS [ESI, M+1]: 198.0.

¹H NMR (400 MHz, DMSO-d6) δ=7.36-7.27 (m, 5H), 6.31-5.87 (m, 1H), 2.99 (br d, J=6.4 Hz, 2H), 2.85-2.77 (m, 2H), 2.25 (br dd, J=7.2, 13.6 Hz, 1H), 2.08 (br s, 1H).

### Step 5: methyl 6-[3-(difluoromethyl)-3-phenyl-pyrrolidin-1-yl]-8-methyl-[1,2,4]triazolo[1,5-a]pyridine-2-carboxylate

A mixture of 3-(difluoromethyl)-3-phenyl-pyrrolidine (280 mg, 1.42 mmol, 1 eq) methyl 6-bromo-8-methyl-[1,2,4]triazolo[1,5-a]pyridine-2-carboxylate (460.13 mg, 1.70 mmol, 1.2 eq), Pd-PEPPSI-IHeptCl (41.43 mg, 42.59 μmol, 0.03 eq) and Cs₂CO₃ (1.39 g, 4.26 mmol, 3 eq) in dioxane (15 mL) was degassed and purged with N₂ for 3 times, and then the mixture was stirred at 100° C. for 10 h under N₂ atmosphere. LCMS showed starting material was consumed and one main peak with desired mass was detected. The mixture was quenched with H₂O (10 mL) and extracted with EtOAc (20 mL×3), the organic phase was dried, filtered, and concentrated under pressure to give the product. The residue was purified by flash silica gel chromatography (ISCO®; 20 g SepaFlash® Silica Flash Column, Eluent of 0~60% Ethyl acetate/Petroleum ether gradient) to give compound methyl 6-[3-(difluoromethyl)-3-phenyl-pyrrolidin-1-yl]-8-methyl-[1,2,4]triazolo[1,5-a]pyridine-2-carboxylate (0.25 g, 46% yield) as a yellow solid.

LC-MS [ESI, M+1]: 387.0.

¹H NMR (400 MHz, CHLOROFORM-d) δ=7.73 (s, 1H), 7.47-7.29 (m, 5H), 7.07 (s, 1H), 5.92 (t, J=56.4 Hz, 1H), 4.13-4.03 (m, 3H), 4.03-3.98 (m, 1H), 3.72 (br d, J=9.6 Hz, 1H), 3.58 (br d, J=8.4 Hz, 1H), 3.45 (dt, J=3.2, 8.8 Hz, 1H), 2.84-2.76 (m, 1H), 2.70 (s, 3H), 2.53 (td, J=8.4, 13.2 Hz, 1H).

### Step 6: methyl 6-[(3R)-3-(difluoromethyl)-3-phenyl-pyrrolidin-1-yl]-8-methyl-[1,2,4]triazolo[1,5-a]pyridine-2-carboxylate

The methyl 6-[3-(difluoromethyl)-3-phenyl-pyrrolidin-1-yl]-8-methyl-[1,2,4]triazolo[1,5-a]pyridine-2-carboxylate (150 mg, 388.2 μmol, 1 eq) was purified by SFC purification (column: DAICEL CHIRALPAK AD (250 mm*30 mm, 10 um);mobile phase: [C02-EtOH (0.1% NH3·H₂O)]; B %: 55%, isocratic elution mode), the mixture was concentrated and lophilizated to give compound methyl 6-[(3R)-3-(difluoromethyl)-3-phenyl-pyrrolidin-1-yl]-8-methyl-[1,2,4]triazolo[1,5-a]pyridine-2-carboxylate (65 mg, 44% yield) as a white solid.

LC-MS [ESI, M+1]: 387.1

### Step 7: 6-[(3R)-3-(difluoromethyl)-3-phenyl-pyrrolidin-1-yl]-8-methyl-[1,2,4]triazolo[1,5-a]pyridine-2-carboxylic acid

To a solution of methyl 6-[(3R)-3-(difluoromethyl)-3-phenyl-pyrrolidin-1-yl]-8-methyl-[1,2,4]triazolo[1,5-a]pyridine2-carboxylate (20 mg, 51.76 μmol, 1 eq) in THF (1 mL) was added LiOH·H₂O (6.52 mg, 155.28 μmol, 3 eq) in H₂O (0.3 mL). The mixture was stirred at 25° C. for 1 h. LCMS showed starting material was consumed and one main peak with desired mass was detected. The mixture was adjusted pH around 7 with dilute HCl (2 M, 0.5 mL), and extracted with EtOAc (10 mL×3), the organic phase was washed with saturated NaCl (10 mL×1), dried, filtered, and concentrated under pressure to give compound 6-[(3R)-3-(difluoromethyl)-3-phenyl-pyrrolidin-1-yl]-8-methyl-[1,2,4]triazolo[1,5-a]pyridine-2-carboxylic acid (18 mg, 93% yield) as a yellow solid.

LC-MS [ESI, M+1]: 373.0.

### Step 8: 6-[(3R)-3-(difluoromethyl)-3-phenyl-pyrrolidin-1-yl]-N-(2-hydroxy-2-methyl-propyl)-8-methyl-[1,2,4]triazolo[1,5-a]pyridine-2-carboxamide

To a solution of 6-[(3R)-3-(difluoromethyl)-3-phenyl-pyrrolidin-1-yl]-8-methyl-[1,2,4]triazolo[1,5-a]pyridine-2-carboxylic acid (18 mg, 48.34 μmol, 1 eq) and 1-amino-2-methyl-propan-2-ol (4.74 mg, 53.17 μmol, 1.1 eq) in DMF (1 mL) was added DIEA (31.24 mg, 241.70 μmol, 42.10 μL, 5 eq) and HATU (55.14 mg, 145.02 μmol, 3 eq). The mixture was stirred at 25° C. for 0.5 h. LCMS showed starting material was consumed and one main peak with desired mass was detected. The mixture was filtered. The residue was purified by prep-HPLC ((column: Welch Ultimate C18 150*25 mm*5 um; mobile phase: [water (FA)—ACN]; gradient:30%-60% B over 10 min) to give compound 6-[(3R)-3-(difluoromethyl)-3-phenyl-pyrrolidin-1-yl]-N-(2-hydroxy-2-methyl-propyl)-8-methyl-[1,2,4]triazolo[1,5-a]pyridine-2-carboxamide (16.99 mg, 7900 yield) as a white solid.

LC-MS [ESI, M]: 443.9.

¹H NMR (400 MHz, CHLOROFORM-d) δ=7.80 (br s, 1H), 7.72 (br s, 1H), 7.46-7.26 (m, 5H), 7.06 (br s, 1H), 5.93 (br t, J=56.8 Hz, 1H), 4.01 (br d, J=8.8 Hz, 1H), 3.71 (br d, J=9.2 Hz, 1H), 3.64-3.40 (m, 4H), 2.90-2.76 (m, 1H), 2.70-2.58 (m, 3H), 2.51 (br s, 2H), 1.33 (br d, J=2.1 Hz, 6H).

| Cmpd. No. | Synthesis | LC/MS | ¹H NMR |
|---|---|---|---|
| 327 | Similar to | m/z 443.9 | ¹H NMR (400 MHz, CHLOROFORM-d) δ = 7.83 (br |
| | Example 28 | [M + H]⁺ | s, 1H), 7.73 (br s, 1H), 7.51-7.29 (m, 5H), 7.09 (br |
| | | | s, 1H), 5.93 (br t, J = 56.8 Hz, 1H), 4.02 (br d, J = 9.2 |
| | | | Hz, 1H), 3.72 (br d, J = 8.0 Hz, 1H), 3.64-3.37 (m, |
| | | | 4H), 2.81 (br s, 1H), 2.66 (s, 3H), 2.50 (br s, 2H), 1.33 |
| | | | (s, 6H) |
| 328 | Similar to | m/z 386.1 | ¹H NMR (400 MHz, CHLOROFORM-d) δ = 7.70 (s, |
| | Example 28 | [M + H]⁺ | 1H), 7.39 (br dd, J = 7.6, 17.4 Hz, 4H), 7.35-7.31 |
| | | | (m, 2H), 7.03 (s, 1H), 5.91 (t, J = 56.4 Hz, 1H), 3.99 |
| | | | (d, J = 9.96 Hz, 1H), 3.69 (br d, J = 9.6 Hz, 1H), 3.55 |
| | | | (br d, J = 8.4 Hz, 1H), 3.48-3.38 (m, 1H), 3.07 (d, |
| | | | J = 5.2 Hz, 3H), 2.86-2.74 (m, 1H), 2.62 (s, 3H), |
| | | | 2.55-2.46 (m, 1H) |
| 329 | Similar to | m/z 386.1 | ¹H NMR (400 MHz, CHLOROFORM-d) δ = 7.71 (s, |
| | Example 28 | [M + H]⁺ | 1H), 7.41 (d, J = 7.6 Hz, 2H), 7.39-7.29 (m, 4H), |
| | | | 7.04 (s, 1H), 5.92 (t, J = 56.4 Hz, 1H), 4.00 (d, J = 9.6 |
| | | | Hz, 1H), 3.70 (br d, J = 9.6 Hz, 1H), 3.62-3.52 (m, |
| | | | 1H), 3.49-3.40 (m, 1H), 3.07 (d, J = 5.2 Hz, 3H), |
| | | | 2.87-2.76 (m, 1H), 2.63 (s, 3H), 2.57-2.47 (m, 1H) |
| 330 | Similar to | m/z 576.1 | ¹H NMR (400 MHz, CHLOROFORM-d) δ = 8.30 (br |
| | Example 28 | [M + H]⁺ | s, 1H), 7.63 (br s, 3H), 7.46-7.28 (m, 4H), 6.96 (br |
| | | | s, 1H), 6.84 (br s, 1H), 5.90 (br t, J = 56.4 Hz, 1H), |
| | | | 4.44-4.28 (m, 1H), 4.15 (br dd, J = 3.6, 5.6 Hz, 2H), |
| | | | 3.95 (br d, J = 9.2 Hz, 1H), 3.63 (br d, J = 9.6 Hz, |
| | | | 1H), 3.55-3.46 (m, 1H), 3.37 (br d, J = 1.6 Hz, 1H), |
| | | | 3.28 (br s, 2H), 2.75 (br s, 1H), 2.63-2.37 (m, 4H), |
| | | | 2.19 (br d, J = 3.6 Hz, 2H), 1.88-1.49 (m, 2H) |
| 331 | Similar to | m/z 582.2 | ¹H NMR (400 MHz, CHLOROFORM-d) δ = 7.70- |
| | Example 28 | [M + H]⁺ | 7.61 (m, 2H), 7.48 (t, J = 6.6 Hz, 1H), 7.44-7.38 (m, |
| | | | 2H), 7.35-7.28 (m, 3H), 7.02 (br d, J = 9.8 Hz, 1H), |
| | | | 6.62 (t, J = 8.6 Hz, 1H), 4.20 (br t, J = 6.4 Hz, 1H), |
| | | | 4.01 (s, 1H), 3.91 (br t, J = 6.9 Hz, 1H), 3.87-3.77 |
| | | | (m, 2H), 3.73 (dd, J = 2.1, 8.1 Hz, 2H), 3.69-3.60 |
| | | | (m, 2H), 3.60-3.49 (m, 4H), 3.44-3.37 (m, 1H), 2.64 |
| | | | (br d, J = 10.8 Hz, 3H), 2.60-2.54 (m, 1H), 2.37- |
| | | | 2.31 (m, 1H), 2.16-2.04 (m, 4H) |
| 332 | Similar to | m/z 582.2 | ¹H NMR (400 MHz, CHLOROFORM-d) δ = 7.70- |
| | Example 28 | [M + H]+ | 7.61 (m, 2H), 7.48 (t, J = 6.8 Hz, 1H), 7.45-7.37 (m, |
| | | | 2H), 7.35-7.27 (m, 3H), 7.02 (br d, J = 9.9 Hz, 1H), |
| | | | 6.62 (t, J = 8.0 Hz, 1H), 4.26-4.13 (m, 1H), 4.01 (s, |
| | | | 1H), 3.91 (t, J = 7.2 Hz, 1H), 3.86-3.77 (m, 2H), 3.73 |
| | | | (dd, J = 2.0, 8.0 Hz, 2H), 3.68-3.56 (m, 3H), 3.56- |
| | | | 3.46 (m, 3H), 3.44-3.35 (m, 1H), 2.67-2.54 (m, 4H), |
| | | | 2.38-2.30 (m, 1H), 2.18-2.04 (m, 4H) |

### Example 29

### 3-(hydroxymethyl)-N, 4-dimethyl-6-[(3R)-3-methyl-3-phenyl-pyrrolidin-1-yl]pyrazolo[1,5-a]pyridine-2-carboxamide (Compound 29)

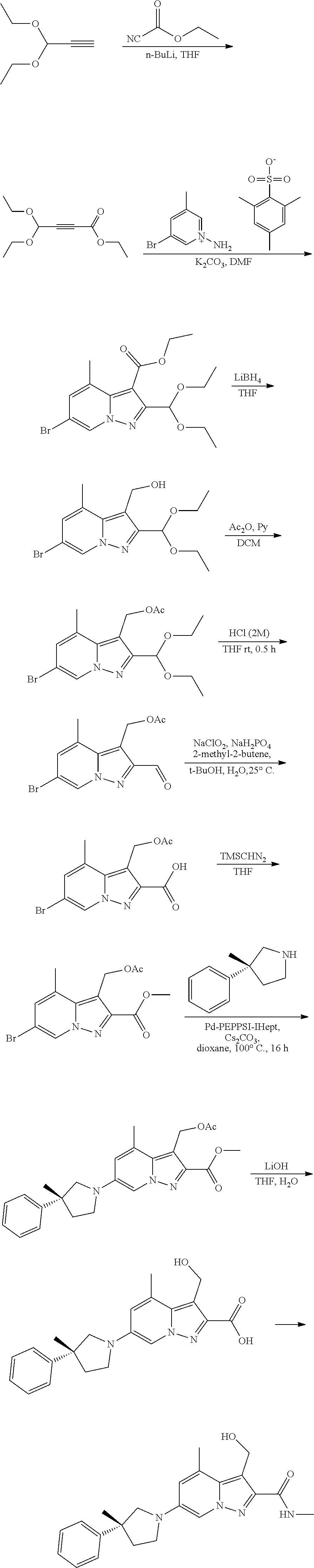

### Step 1: 3,3-dimethoxyprop-1-yne

To a solution of 3,3-diethoxyprop-1-yne (20 g, 156.04 mmol, 22.37 mL, 1 eq) in THF (200 mL) was added n-BuLi (2.5 M, 93.63 mL, 1.5 eq) at −78° C. The mixture was stirred at −78° C. for 1 h, then ethyl cyanoformate (20.10 g, 202.86 mmol, 19.88 mL, 1.3 eq) was added and stirred at −78° C. for 0.5 h. The mixture was slowly warmed to 25° C. for 2 h under N₂ atmosphere. TLC (PE:EtOAc=5:1, Rf=0.3) indicated reactant was consumed and one new spot formed. The reaction was filtered and the filtered concentrated under reduced pressure to give a residue. The residue was purified by flash silica gel chromatography (ISCO®; 80 g SepaFlash® Silica Flash Column, Eluent of 0-20% Ethyl acetate/Petroleum ether gradient to give compound ethyl 4, 4-diethoxybut-2-ynoate (17.5 g, 56% yield) was obtained as a yellow oil.

¹H NMR (400 MHz, CHLOROFORM-d) δ=5.33 (s, 1H), 4.34-4.16 (m, 1H), 4.09 (q, J=7.2 Hz, 2H), 3.72 (qd, J=7.2, 9.2 Hz, 1H), 3.60 (qd, J=7.2, 9.2 Hz, 1H), 1.40-1.18 (m, 9H).

### Step 2: ethyl 6-bromo-2-(diethoxymethyl)-4-methyl-pyrazolo[1,5-a]pyridine-3-carboxylate

To a solution of ethyl 4,4-diethoxybut-2-ynoate (10 g, 49.94 mmol, 1 eq) and 3-bromo-5-methyl-pyridin-1-ium-1-amine;2,4,6-trimethylbenzenesulfonate (15.47 g, 39.95 mmol, 0.8 eq) in DMF (100 mL) was added K₂CO₃ (13.80 g, 99.88 mmol, 2 eq). The mixture was stirred at 70° C. for 16 h. TLC (PE:EtOAc=5:1, Rf=0.3) indicated reactant was consumed and one new spot formed. The reaction was filtered and the filtered concentrated under reduced pressure to give a residue. The residue was purified by flash silica gel chromatography (ISCO®; 220 g SepaFlash® Silica Flash Column, Eluent of 0-5% PE:EtOAc ether gradient to give compound dimethylethyl 6-bromo-2-(diethoxymethyl)-4-methyl-pyrazolo[1,5-a]pyridine-3-carboxylate (6 g, 31% yield) was obtained as a yellow solid.

¹H NMR (400 MHz, CHLOROFORM-d) δ=8.54 (s, 1H), 7.11 (s, 1H), 5.68 (s, 2H), 4.02 (s, 3H), 2.64 (s, 3H), 2.07 (s, 3H).

### Step 3:6-bromo-2-(diethoxymethyl)-4-methyl-pyrazolo[1,5-a]pyridine-3-carboxylate

To a solution of ethyl 6-bromo-2-(diethoxymethyl)-4-methyl-pyrazolo[1,5-a]pyridine-3-carboxylate (5 g, 12.98 mmol, 1 eq) in THF (80 mL) was added LiBH₄ (2 M, 19.47 mL, 3 eq). The mixture was stirred at 0-25° C. for 16 h. LCMS showed starting material was consumed and the desired mass was detected. On completion, the reaction mixture was quenched by addition NH₄Cl (80 mL) at 0° C. The reaction was filtered and the filtered concentrated under reduced pressure to give a residue. The residue was purified by flash silica gel chromatography (ISCO®; 120 g Sepa Flash® Silica Flash Column, Eluent of 0~18% PE:EtOAc ether gradient) to give compound dimethyl 4-[[tert-butyl(diphenyl)silyl]oxymethyl]-2-oxabicyclo[2.1.1]hexane-1,5-dicarboxylate (7 g, 15% yield) as a colorless oil.

¹H NMR (400 MHz, CHLOROFORM-d) δ=7.69-7.61 (m, 4H), 7.48-7.38 (m, 6H), 4.07 (d, J=11.2 Hz, 1H), 3.93 (d, J=11.2 Hz, 1H), 3.90 (d, J=6.0 Hz, 1H), 3.85 (s, 3H), 3.78 (d, J=6.0 Hz, 1H), 3.67 (s, 3H), 3.14 (s, 1H), 2.14 (d, J=7.2 Hz, 1H), 1.81 (d, J=7.2 Hz, 1H), 1.08 (s, 9H).

### Step 4: methyl 1-(6-bromo-8-methyl-[1,2,4]triazolo[1,5-a]pyridin-2-yl)-4-(((tert-butyldiphenylsilyl)oxy)methyl)-2-oxabicyclo[2.1.1]hexane-5-carboxylate

To a solution of 5-bromo-3-methyl-pyridin-1-ium-1,2-diamine;2,4,6-trimethylbenzenesulfonate (6.70 g, 16.64 mmol, 1.3 eq) and dimethyl 4-[[tert-butyl(diphenyl)silyl]oxymethyl]-2-oxabicyclo[2.1.1]hexane-1,5-dicarboxylate (6 g, 12.80 mmol, 1 eq) in MeOH (300 mL) was added NaOH (768.17 mg, 19.21 mmol, 1.5 eq) The mixture was stirred at 25° C. for 12 h. LCMS showed starting material was consumed and the desired mass was detected. The mixture was quenched with H₂O (300 mL) and extracted with EtOAc (300 mL×3), the organic phase was dried, filtered, and concentrated under pressure to give the product. The reaction was concentrated under reduced pressure to give a residue. The residue was purified by flash silica gel chromatography (ISCO®; 40 g SepaFlash® Silica Flash Column, Eluent of 0~20% PE:EtOAc ether gradient) to give compound [6-bromo-2-(diethoxymethyl)-4-methyl-pyrazolo[1,5-a]pyridin-3-yl]methanol (4 g, 90% yield) was obtained as a yellow solid.

LC-MS [ESI, M+23]: 346.8

### Step 5: [6-bromo-2-(diethoxymethyl)-4-methyl-pyrazolo[1,5-a]pyridin-3-yl]methyl acetate

To a solution of [6-bromo-2-(diethoxymethyl)-4-methyl-pyrazolo[1,5-a]pyridin-3-yl]methanol (3.7 g, 10.78 mmol, 1 eq) in DCM (40 mL) was added TEA (2.18 g, 21.56 mmol, 3.00 mL, 2 eq) and acetyl acetate (2.20 g, 21.56 mmol, 2.02 mL, 2 eq). The mixture was stirred at 25° C. for 16 h. TLC (PE:EtOAc=3:1, Rf=0.3) indicated reactant was consumed and one new spot formed. The mixture was concentrated in vacuum and extracted with EtOAc (40 mL×3). The combined organic layer was washed with Sat. brine (40 mL), dried over sodium sulfate, concentrated in vacuum to afford crude. The residue was purified by column chromatography (SiO2, PE:EtOAc=5:1 to 3:1) to give compound (6-bromo-2-formyl-4-methyl-pyrazolo[1,5-a]pyridin-3-yl)methyl acetate (2 g, 83% yield) was obtained as a white solid.

¹H NMR (400 MHz, CHLOROFORM-d) δ=8.46 (s, 1H), 7.01 (s, 1H), 5.84 (s, 1H), 5.46 (s, 2H), 3.77-3.67 (m, 2H), 3.67-3.58 (m, 2H), 2.59 (s, 3H), 2.06 (s, 3H), 1.25 (t, J=7.2 Hz, 6H).

### Step 6: 3-(acetoxymethyl)-6-bromo-4-methyl-pyrazolo[1,5-a]pyridine-2-carboxylic acid

To a solution of (6-bromo-2-formyl-4-methyl-pyrazolo[1,5-a]pyridin-3-yl)methyl acetate (1.3 g, 4.18 mmol, 1 eq) in 2-methyl-2-propanol (24 mL), was added 2-methylbut-2-ene (43.96 g, 626.75 mmol, 66.40 mL, 150 eq) in THF (24 mL), followed by addition of sodium;dihydrogen phosphate (5.01 g, 41.78 mmol, 10 eq) and sodium;chlorite (1.89 g, 20.89 mmol, 5 eq) in Water (10 mL) at 0° C. The reaction mixture was stirred at 25° C. for 30 minutes. LCMS showed starting material was consumed and the desired mass was detected. The mixture was concentrated in vacuum and extracted with EtOAc (40 mL×3). The combined organic layer was washed with Sat. brine (40 mL), dried over sodium sulfate, concentrated in vacuum to afford crude. The residue was purified by column chromatography (SiO₂, PE:EtOAc=10:1 to 0:1) to give compound 3-(acetoxymethyl)-6-bromo-4-methyl-pyrazolo[1,5-a]pyridine-2-carboxylic acid (1 g, 74% yield) was obtained as a white solid.

¹H NMR (400 MHz, CHLOROFORM-d) δ=8.60 (s, 1H), 7.45 (s, 1H), 4.51-4.35 (m, 1H), 4.25 (d, J=6.4 Hz, 1H), 3.78-3.73 (m, 1H), 3.71-3.60 (m, 3H), 2.73-2.62 (m, 4H), 2.40-2.30 (m, 1H).

### Step 7: methyl 3-(acetoxymethyl)-6-bromo-4-methyl-pyrazolo[1,5-a]pyridine-2-carboxylate

To a solution of 3-(acetoxymethyl)-6-bromo-4-methyl-pyrazolo[1,5-a]pyridine-2-carboxylic acid (1 g, 3.06 mmol, 1 eq) in THF (5 mL) was added TMSCHN₂ (3.49 g, 30.57 mmol, 10 eq) and. The mixture was stirred at 0° C. for 3 h. LCMS showed starting material was consumed and one main peak with desired mass was detected. The mixture was concentrated in vacuum and extracted with EtOAc (5 mL×3). The combined organic layer was washed with Sat. brine (5 mL), dried over sodium sulfate, concentrated in vacuum to afford crude. The residue was purified by column chromatography (SiO₂, PE:EtOAc=10:1 to 3:1) to give compound methyl 3-(acetoxymethyl)-6-bromo-4-methyl-pyrazolo[1,5-a]pyridine-2-carboxylate (800 mg, 77% yield) was obtained as a white solid.

LC-MS [ESI, M+23]: 365.0

¹H NMR (400 MHz, CHLOROFORM-d) δ=8.49 (d, J=1.2 Hz, 1H), 7.33 (d, J=1.2 Hz, 1H), 4.27 (d, J=6.4 Hz, 1H), 4.08 (d, J=6.4 Hz, 1H), 3.56 (s, 3H), 3.53 (s, 1H), 2.58 (s, 3H), 2.49 (d, J=7.2 Hz, 1H), 2.18 (dd, J=1.2, 7.2 Hz, 1H), 1.45 (s, 9H).

### Step 8: methyl 3-(acetoxymethyl)-6-bromo-4-methyl-pyrazolo[1,5-a]pyridine-2-carboxylate

A mixture of methyl 3-(acetoxymethyl)-6-bromo-4-methyl-pyrazolo[1,5-a]pyridine-2-carboxylate (400 mg, 1.17 mmol, 1 eq), (3R)-3-methyl-3-phenyl-pyrrolidine (189.05 mg, 1.17 mmol, 1 eq), 1,3-bis[2,6-bis(1-propylbutyl)phenyl]-4,5-dichloro-2H-imidazol-1-ium-2-ide;3-chloropyridine;dichloropalladium (114.06 mg, 117.25 μmol, 0.1 eq) and dicesium;carbonate (1.15 g, 3.52 mmol, 3 eq) in 1,4-dioxane (4 mL) was degassed and purged with N₂ for 3 times, and then the mixture was stirred at 100° C. for 2 h under N₂ atmosphere. LCMS showed starting material was consumed and one main peak with desired mass was detected. The mixture was concentrated in vacuum and extracted with EtOAc (5 mL×3). The combined organic layer was washed with Sat. brine (5 mL), dried over sodium sulfate, concentrated in vacuum to afford crude. The mixture was filtered, and concentrated under pressure to give the product. The residue was purified by flash silica gel chromatography (ISCO®; 40 g SepaFlash® Silica Flash Column, Eluent of 0~50% PE:EtOAc ether gradient) to give compound methyl 3-(acetoxymethyl)-4-methyl-6-[(3R)-3-methyl-3-phenyl-pyrrolidin-1-yl]pyrazolo[1,5-a]pyridine2-carboxylate (300 mg, 61% yield) was obtained as a yellow solid.

LC-MS [ESI, M+1]: 422.2.

### Step 9: 3-(hydroxymethyl)-4-methyl-6-[(3R)-3-methyl-3-phenyl-pyrrolidin-1-yl]pyrazolo[1,5-a]pyridine-2-carboxylic acid

To a solution of methyl 3-(acetoxymethyl)-4-methyl-6-[(3R)-3-methyl-3-phenyl-pyrrolidin-1-yl]pyrazolo[1,5-a]pyridine-2-carboxylate (100 mg, 237.25 μmol, 1 eq) in THF (1 mL) and Water (1 mL) was added LiOH·H₂O (29.87 mg, 711.76 μmol, 3 eq). The mixture was stirred at 25° C. for 1 h. LCMS showed starting material was consumed and one main peak with desired mass was detected. The reaction was filtered and the filtered concentrated under reduced pressure to give compound 3-(hydroxymethyl)-4-methyl-6-[(3R)-3-methyl-3-phenyl-pyrrolidin-1-yl]pyrazolo[1,5-a]pyridine-2-carboxylic acid (80 mg, 92% yield) was obtained as a yellow solid.

LC-MS [ESI, M+1]: 366.1

### Step 10: 3-(hydroxymethyl)-N, 4-dimethyl-6-[(3R)-3-methyl-3-phenyl-pyrrolidin-1-yl]pyrazolo[1,5-a]pyridine-2-carboxamide

To a solution of 3-(hydroxymethyl)-4-methyl-6-[(3R)-3-methyl-3-phenyl-pyrrolidin-1-yl]pyrazolo[1,5-a]pyridine-2-carboxylic acid (80 mg, 218.92 μmol, 1 eq) and methanamine; hydrochloride (34.15 mg, 328.39 μmol, 1.5 eq, HCl) in DMF (2 mL) was added HATU (124.86 mg, 328.39 μmol, 1.5 eq) and DIEA (84.88 mg, 656.77 μmol, 114.40 μL, 3 eq). The mixture was stirred at 25° C. for 16 h. LCMS showed starting material was consumed and one main peak with desired mass was detected. The mixture was concentrated in vacuum and extracted with EtOAc (5 mL×3). The combined organic layer was washed with Sat. brine (5 mL), dried over sodium sulfate, concentrated in vacuum to afford crude. The residue was purified by prep-HPLC purification (mobile phase: [(column: CD02-Waters Xbidge BEH C18 150*25*10 um; mobile phase: [water(NH3H2O)—ACN]; gradient:42%-62% B over 12 min), the mixture was concentrated and lyophilized to give compound 3-(hydroxymethyl)-N, 4-dimethyl-6-[(3R)-3-methyl-3-phenyl-pyrrolidin-1-yl]pyrazolo[1,5-a]pyridine-2-carboxamide (23.15 mg, 26% yield) was obtained as a white solid.

LC-MS [ESI, M]: 361.0.

¹H NMR (400 MHz, DMSO-d6) δ=8.14 (br d, J=4.8 Hz, 1H), 7.54 (s, 1H), 7.40-7.4 (m, 5H), 7.2-7.2 (m, 1H), 6.90 (s, 1H), 5.00-4.91 (m, 3H), 3.61-3.54 (m, 1H), 3.53-3.42 (m, 2H), 3.35 (br s, 1H), 2.79 (d, J=4.8 Hz, 3H), 2.64 (s, 3H), 2.27-2.20 (m, 2H), 1.37-1.33 (m, 3H).

| Cmpd. | | | |
|---|---|---|---|
| No. | Synthesis | LC/MS | ¹H NMR |
| 333 | Similar to | m/z | ¹H NMR (400 MHz, CHLOROFORM-d) δ = |
| | Example 29 | 361.1 | 10.23 (s, 1H), 8.46 (s, 1H), 7.04 (s, 1H), |
| | | [M + H]+ | 5.17 (s, 2H), 2.69 (s, 3H), 1.62-1.59 (m, 3H) |

### Example 30

### 8-(1-hydroxy-1-methyl-ethyl)-N-methyl-6-[(3R)-3-methyl-3-phenyl-pyrrolidin-1-yl]-[1,2,4]triazolo[1,5-a]pyridine-2-carboxamide (Compound 30)

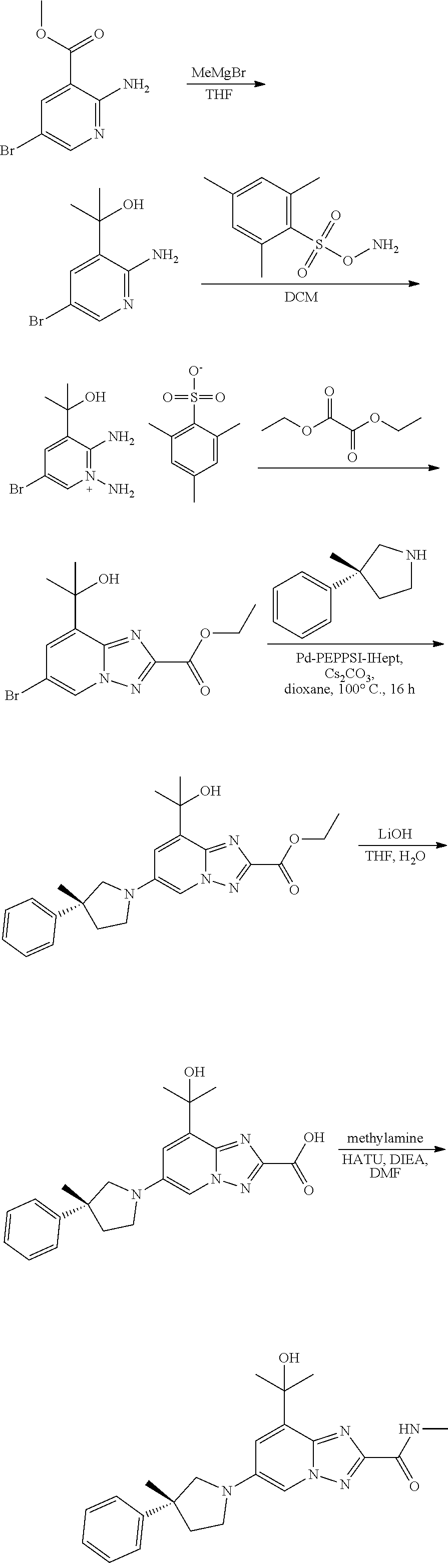

### Step 1: 2-(2-amino-5-bromo-3-pyridyl)propan-2-ol

A mixture of methyl 2-amino-5-bromo-pyridine-3-carboxylate (5 g, 21.64 mmol, 1 eq) in THF (75 mL) was degassed and purged with N₂ for 3 times, and then cooled to −20° C. and MeMgBr (3 M, 36.07 mL, 5 eq) was added via syringe over 30 minutes. The reaction mixture was warmed to 25° C. and stirred for 16 h. LCMS showed starting material was consumed and the desired mass was detected. The reaction mixture was cooled to −20° C. and quenched slowly with saturated ammonium chloride (300 mL). Water and EtOAc (100 mL×2) were added and the layers were separated. The collected organics were washed with saturated NaCl, dried over Na2SO4, filtered and concentrated to dryness to residue. The residue was purified by column chromatography (SiO2, PE:EtOAc=1:0 to 1:1) to give compound 2-(2-amino-5-bromo-3-pyridyl) propan-2-ol (4.8 g, 91% yield) was obtained as yellow solid.

LC-MS [ESI, M+1]:232.9.

### Step 2: 2-(1,2-diamino-5-bromo-pyridin-1-ium-3-yl)propan-2-ol;2,4,6-trimethylbenzenesulfonate

To a mixture of amino 2,4,6-trimethylbenzenesulfonate (5 g, 23.23 mmol, 1.1 eq) in DCM (30 mL) was added dropwise a solution of 2-(2-amino-5-bromo-3-pyridyl)propan-2-ol (4.8 g, 20.77 mmol, 1 eq) in DCM (50 mL) at 0° C., then the mixture was stirred at 0° C. for 0.5 h. LCMS showed starting material was consumed and the desired mass was detected. The reaction mixture was concentrated in vacuum to give compound 2-(1, 2-diamino-5-bromo-pyridin-1-ium-3-yl) propan-2-ol; 2, 4, 6-trimethylbenzenesulfonate (7 g, crude) was obtained as a brown oil.

¹H NMR (400 MHz, DMSO-d6) δ=8.38 (br s, 1H), 7.86 (s, 1H), 6.92 (s, 2H), 6.75 (s, 2H), 6.29 (br s, 1H), 5.76 (s, 1H), 2.50 (s, 6H), 2.18 (s, 3H), 1.54 (s, 3H), 1.49 (s, 3H)

LC-MS [ESI, M+1]:247.9.

### Step 3: ethyl 6-bromo-8-(1-hydroxy-1-methyl-ethyl)-[1,2,4]triazolo[1,5-a]pyridine-2-carboxylate

To a solution of 2-(1,2-diamino-5-bromo-pyridin-1-ium-3-yl) propan-2-ol;2,4,6-trimethylbenzenesulfonate (2 g, 4.48 mmol, 1 eq) and diethyl oxalate (785.78 mg, 5.38 mmol, 734.37 μL, 1.2 eq) in EtOH (3 mL) was added NaOH (268.84 mg, 6.72 mmol, 1.5 eq). The mixture was stirred at 60° C. for 2 h. LCMS showed starting material was consumed and the desired mass was detected. The reaction mixture was added silica gel and concentrated under reduced pressure to give a residue. The residue was purified by column chromatography (SiO₂, PE:EtOAc=1:0 to 0:1) to give product. The crude product was purified by re-crystallization from ACN (5 mL) at 25° C. to give compound ethyl 6-bromo-8-(1-hydroxy-1-methyl-ethyl)-[1, 2,4] triazolo [1, 5-a]pyridine-2-carboxylate (300 mg, 20% yield) was obtained as white solid.

¹H NMR (400 MHz, DMSO-d6) δ=9.37 (s, 1H), 7.85 (s, 1H), 5.67 (s, 1H), 4.41 (q, J=7.2 Hz, 2H), 1.67 (s, 6H), 1.35 (t, J=7.2 Hz, 3H)

LC-MS [ESI, M+1]:327.8.

### Step 4: ethyl 8-(1-hydroxy-1-methyl-ethyl)-6-[(3R)-3-methyl-3-phenyl-pyrrolidin-1-yl]-[1,2,4]triazolo[1,5-a]pyridine-2-carboxylate

A mixture of (3R)-3-methyl-3-phenyl-pyrrolidine (88.44 mg, 548.51 μmol, 1.2 eq), ethyl 6-bromo-8-(1-hydroxy-1-methyl-ethyl)-[1,2,4]triazolo[1,5-a]pyridine-2-carboxylate (150 mg, 457.09 μmol, 1 eq), Cs₂CO₃ (446.79 mg, 1.37 mmol, 3 eq), 1,3-bis[2,6-bis(1-propylbutyl)phenyl]-4,5-dichloro-2H-imidazol-1-ium-2-ide;3-chloropyridine; dichloropalladium (44.46 mg, 45.71 μmol, 0.1 eq) in DIOXANE (2 mL) was degassed and purged with N₂ for 3 times, and then the mixture was stirred at 100° C. for 16 h under N₂ atmosphere. LCMS showed starting material was consumed and the desired mass was detected. The mixture was filtered and concentrated to give a residue. The residue was purified by prep-TLC (PE:EtOAc=1:1) to give compound ethyl 8-(1-hydroxy-1-methyl-ethyl)-6-[(3R)-3-methyl-3-phenyl-pyrrolidin-1-yl]-[1, 2, 4]triazolo [1, 5-a]pyridine-2-carboxylate (100 mg, 48% yield) was obtained as green oil.

LC-MS [ESI, M+1]: 408.9.

¹H NMR (400 MHz, CHLOROFORM-d) δ=7.72 (d, J=1.6 Hz, 1H), 7.41-7.28 (m, 5H), 7.19 (d, J=2.0 Hz, 1H), 4.53 (q, J=7.2 Hz, 2H), 3.63 (d, J=8.8 Hz, 1H), 3.59-3.52 (m, 1H), 3.50 (d, J=8.8 Hz, 1H), 3.44 (dt, J=3.6, 8.8 Hz, 1H), 2.40 (td, J=8.4, 12.0 Hz, 1H), 2.31-2.24 (m, 1H), 1.80 (s, 6H), 1.50-1.45 (m, 6H)

### Step 5: 8-(1-hydroxy-1-methyl-ethyl)-6-[(3R)-3-methyl-3-phenyl-pyrrolidin-1-yl]-[1,2,4]triazolo[1,5-a]pyridine-2-carboxylic acid

To a solution of ethyl 8-(1-hydroxy-1-methyl-ethyl)-6-[(3R)-3-methyl-3-phenyl-pyrrolidin-1-yl]-[1,2,4]triazolo[1,5-a]pyridine-2-carboxylate (50 mg, 122.40 μmol, 1 eq) in THF (1 mL) and H₂O (0.5 mL) was added LiOH·H₂O (15.41 mg, 367.20 μmol, 3 eq). The mixture was stirred at 25° C. for 1 h. LCMS showed starting material was consumed and one main peak with desired mass was detected. The reaction mixture was partitioned between EtOAc (5 mL×3) and water (10 mL), the combined organic phase was dried over anhydrous sodium sulfate, filtered and concentrated to give compound 8-(1-hydroxy-1-methyl-ethyl)-6-[(3R)-3-methyl-3-phenyl-pyrrolidin-1-yl]-[1,2,4]triazolo[1,5-a]pyridine-2-carboxylic acid (50 mg, crude) colorless oil.

LC-MS [ESI, M+1]: 381.1.

### Step 6: 8-(1-hydroxy-1-methyl-ethyl)-N-methyl-6-[(3R)-3-methyl-3-phenyl-pyrrolidin-1-yl]-[1,2,4]triazolo[1,5-a]pyridine-2-carboxamide

To a solution of 8-(1-hydroxy-1-methyl-ethyl)-6-[(3R)-3-methyl-3-phenyl-pyrrolidin-1-yl]-[1,2,4]triazolo[1,5-a]pyridine-2-carboxylic acid (50 mg, 131.43 μmol, 1 eq) and methanamine;hydrochloride (11.54 mg, 170.86 μmol, 1.3 eq) in DMF (1 mL) was added DIEA (50.96 mg, 394.28 μmol, 68.68 μL, 3 eq) and HATU (59.97 mg, 157.71 μmol, 1.2 eq). The mixture was stirred at 25° C. for 1 h. LCMS showed starting material was consumed and one main peak with desired mass was detected. The reaction mixture was partitioned between EtOAc (3 mL×3) and water (5 mL), the combined organic phase was dried over anhydrous sodium sulfate, filtered and concentrated to give a residue. The crude product was purified by reversed-phase HPLC (column: Phenomenex luna C18 150*25 mm*10 um; mobile phase: [water(FA)—ACN]; gradient:37%-67% B over 3 min) to give compound 8-(1-hydroxy-1-methyl-ethyl)-N-methyl-6-[(3R)-3-methyl-3-phenyl-pyrrolidin-1-yl]-[1,2,4]triazolo[1,5-a]pyridine-2-carboxamide (21.85 mg, 42% yield) was obtained as yellow solid.

LC-MS [ESI, M+1]: 394.1

¹H NMR (400 MHz, CHLOROFORM-d) 8=8.15 (s, 1H), 7.72 (d, J=1.6 Hz, 1H), 7.41-7.35 (m, 2H), 7.35-7.28 (m, 3H), 7.27-7.24 (m, 1H), 7.17 (d, J=2.0 Hz, 1H), 3.63 (d, J=8.8 Hz, 1H), 3.56 (q, J=8.0 Hz, 1H), 3.50 (d, J=8.8 Hz, 1H), 3.44 (dt, J=3.6, 8.8 Hz, 1H), 3.09 (d, J=5.2 Hz, 3H), 2.40 (td, J=8.4, 12.0 Hz, 1H), 2.31-2.24 (m, 1H), 1.78 (s, 6H), 1.48 (s, 3H)

| Cmpd. | | | |
|---|---|---|---|
| No. | Synthesis | LC/MS | ¹H NMR |
| 334 | Similar to | m/z 582.2 | ¹H NMR (400 MHz, DMSO-d₆) δ = 8.16 (s, 1H), |
| | Example 30 | [M + H]⁺ | 8.07-7.87 (m, 2H), 7.45-7.30 (m, 5H), 7.27-7.19 |
| | | | (m, 1H), 7.11 (dd, J = 15.2, 2.0 Hz, 1H), 6.74 (dd, |
| | | | J = 16.8, 5.2 Hz, 1H), 5.83-5.32 (m, 1H), 4.83 (d, |
| | | | J = 12.2 Hz, 2H), 3.97 (t, J = 6.4 Hz, 1H), 3.81 (d, |
| | | | J = 3.2 Hz, 1H), 3.65 (dd, J = 19.2, 8.0 Hz, 4H), |
| | | | 3.57-3.51 (m, 4H), 3.42-3.36 (m, 2H), 2.27 (q, J = 6.7 |
| | | | Hz, 2H), 2.16-1.95 (m, 4H), 1.36 (d, J = 8.0 Hz, |
| | | | 3H). |
| 335 | Similar to | m/z 365.2 | ¹H NMR (400 MHz, CHLOROFORM-d) δ = 7.69 |
| | Example 30 | [M + H]⁺ | (s, 1H), 7.41-7.34 (m, 2H), 7.34-7.30 (m, 2H), |
| | | | 7.25 (s, 1H), 7.19 (s, 1H), 4.22 (s, 2H), 3.63 (d, J = |
| | | | 8.8 Hz, 1H), 3.58-3.49 (m, 2H), 3.43 (dt, J = 8.4, |
| | | | 4.0 Hz, 1H), 3.07 (d, J = 5.2 Hz, 3H), 2.47-2.34 |
| | | | (m, 1H), 2.32-2.23 (m, 1H), 1.47 (s, 3H). |
| 336 | Similar to | m/z 380.0 | ¹H NMR (400 MHz, CHLOROFORM-d) δ = 7.69 |
| | Example 30 | [M + H]⁺ | (d, J = 2.0 Hz, 1H), 7.42-7.35 (m, 2H), 7.34-7.28 |
| | | | (m, 3H), 7.27-7.24 (m, 1H), 7.21 (d, J = 2.0 Hz, |
| | | | 1H), 5.33 (q, J = 6.0 Hz, 1H), 3.63 (d, J = 8.8 Hz, |
| | | | 1H), 3.59-3.52 (m, 1H), 3.52-3.49 (m, 1H), 3.44 |
| | | | (dt, J = 3.6, 8.8 Hz, 1H), 3.32 (br d, J = 1.2 Hz, 1H), |
| | | | 3.08 (d, J = 5.2 Hz, 3H), 2.40 (td, J = 8.4, 12.0 Hz, |
| | | | 1H), 2.32-2.23 (m, 1H), 1.70 (d, J = 6.4 Hz, 3H), |
| | | | 1.47 (s, 3H) |
| 337 | Similar to | m/z 378.1 | ¹H NMR (400 MHz, CHLOROFORM-d) δ = 7.69 |
| | Example 30 | [M + H]⁺ | (s, 1H), 7.42-7.31 (m, 5H), 7.25 (br d, J = 1.2 Hz, |
| | | | 1H), 6.99 (s, 1H), 3.61 (d, J = 8.8 Hz, 1H), 3.59- |
| | | | 3.52 (m, 2H), 3.49 (d, J = 8.8 Hz, 1H), 3.44 (dt, J = |
| | | | 3.2, 8.8 Hz, 1H), 3.08 (d, J = 5.2 Hz, 3H), 2.44- |
| | | | 2.34 (m, 1H), 2.31-2.22 (m, 1H), 1.48 (s, 3H), 1.42 |
| | | | (d, J = 6.8 Hz, 6H) |
| 338 | Similar to | m/z 361.2 | ¹H NMR (400 MHz, CHLOROFORM-d) δ = 7.75 |
| | Example 30 | [M + H]⁺ | (d, J = 8.4 Hz, 1H), 7.59 (br d, J = 3.2 Hz, 1H), |
| | | | 7.41-7.28 (m, 5H), 6.02 (d, J = 8.8 Hz, 1H), |
| | | | 4.41-4.11 (m, 4H), 3.08 (d, J = 5.2 Hz, 3H), |
| | | | 2.48-2.36 (m, 1H), 2.34-2.25 (m, 1H), 1.51 (s, 3H) |
| 339 | Similar to | m/z 364.3 | ¹H NMR (400 MHz, CHLOROFORM-d) δ = 8.19 |
| | Example 30 | [M + H]⁺ | (s, 1H), 7.69 (d, J = 1.6 Hz, 1H), 7.41-7.28 (m, |
| | | | 4H), 7.25 (br d, J = 1.6 Hz, 1H), 7.00 (s, 1H), 3.61 |
| | | | (d, J = 8.8 Hz, 1H), 3.54 (q, J = 8.4 Hz, 1H), 3.49 |
| | | | (d, J = 8.8 Hz, 1H), 3.42 (dt, J = 3.6, 8.8 Hz, 1H), |
| | | | 3.07 (d, J = 5.2 Hz, 3H), 3.05-2.96 (m, 2H), 2.39 |
| | | | (td, J = 8.4, 12.0 Hz, 1H), 2.31-2.22 (m, 1H), 1.47 |
| | | | (s, 3H), 1.40 (t, J = 7.6 Hz, 3H) |
| 340 | Similar to | m/z 584.4 | ¹H NMR (400 MHz, CHLOROFORM-d) δ = 7.76- |
| | Example 30 | [M + H]⁺ | 7.66 (m, 2H), 7.54 (d, J = 7.2 Hz, 1H), 7.41-7.35 |
| | | | (m, 2H), 7.35-7.31 (m, 2H), 7.29 (s, 1H), 7.17 (d, J = |
| | | | 2.0 Hz, 1H), 7.15 (br d, J = 8.4 Hz, 1H), 6.99 (d, |
| | | | J = 8.4 Hz, 1H), 4.44-4.25 (m, 4H), 3.63 (d, J = 8.8 |
| | | | Hz, 1H), 3.56 (q, J = 8.0 Hz, 1H), 3.50 (d, J = 8.8 |
| | | | Hz, 1H), 3.44 (dt, J = 3.6, 8.8 Hz, 1H), 3.23-3.14 |
| | | | (m, 2H), 2.41 (td, J = 8.4, 12.4 Hz, 1H), 2.32-2.19 |
| | | | (m, 3H), 1.78 (s, 6H), 1.68 (br d, J = 3.2 Hz, 1H), |
| | | | 1.65 (br d, J = 3.6 Hz, 1H), 1.48 (s, 3H) |
| 341 | Similar to | m/z 585.3 | ¹H NMR (400 MHz, CHLOROFORM-d) δ = 7.64 |
| | Example 30 | [M + H]⁺ | (br s, 2H), 7.33 (br d, J = 7.2 Hz, 2H), 7.24 (br d, |
| | | | J = 6.8 Hz, 4H), 5.36 (br d, J = 6.4 Hz, 1H), |
| | | | 4.59-4.38 (m, 1H), 3.61-3.44 (m, 3H), 3.42-3.24 (m, |
| | | | 3H), 3.22-3.12 (m, 1H), 2.76-2.64 (m, 2H), 2.56- |
| | | | 2.48 (m, 2H), 2.10 (br d, J = 16.8 Hz, 12H), 1.67 |
| | | | (br d, J = 4.8 Hz, 3H), 1.42 (br s, 3H) |
| 342 | Similar to | m/z 570.3 | ¹H NMR (400 MHz, CHLOROFORM-d) δ = 7.75- |
| | Example 30 | [M + H]⁺ | 7.67 (m, 2H), 7.55 (d, J = 7.2 Hz, 1H), 7.42-7.36 |
| | | | (m, 2H), 7.35-7.31 (m, 2H), 7.30 (br s, 1H), 7.25- |
| | | | 7.18 (m, 2H), 7.00 (d, J = 8.8 Hz, 1H), 5.36 (q, J = |
| | | | 6.4 Hz, 1H), 4.45-4.37 (m, 1H), 4.33 (br d, J = 13.6 |
| | | | Hz, 2H), 3.64 (d, J = 8.8 Hz, 1H), 3.61-3.50 (m, |
| | | | 2H), 3.45 (dt, J = 3.6, 8.8 Hz, 1H), 3.20 (br t, J = |
| | | | 11.6 Hz, 2H), 2.47-2.36 (m, 1H), 2.33-2.21 (m, 3H), |
| | | | 1.71 (br d, J = 6.4 Hz, 3H), 1.66 (br d, J = 2.8 |
| | | | Hz, 1H), 1.63 (br d, J = 4.0 Hz, 1H), 1.49 (s, 3H) |
| 343 | Similar to | m/z 582.4 | ¹H NMR (400 MHz, CHLOROFORM-d) δ = 7.73- |
| | Example 30 | [M + H]⁺ | 7.62 (m, 2H), 7.49 (t, J = 6.2 Hz, 1H), 7.41-7.28 |
| | | | (m, 5H), 7.23 (br d, J = 6.4 Hz, 1H), 6.63 (t, J = |
| | | | 8.4Hz, 1H), 5.06 (d, J = 9.2 Hz, 2H), 4.27-4.14 (m, |
| | | | 1H), 4.02 (s, 1H), 3.92 (br t, J = 6.8 Hz, 1H), |
| | | | 3.87-3.75 (m, 1H), 3.70-3.57 (m, 4H), 3.56-3.49 (m, |
| | | | 3H), 3.44 (br dd, J = 3.6, 8.5 Hz, 1H), 2.44-2.36 |
| | | | (m, 1H), 2.28 (br dd, J = 4.4, 11.6 Hz, 1H), 2.16- |
| | | | 2.05 (m, 4H), 1.47 (br d, J = 8.0 Hz, 3H) |
| 344 | Similar to | m/z 472.2 | ¹H NMR (400 MHz, CHLOROFORM-d) δ = 7.70 |
| | Example 30 | [M + H]+ | (s, 1H), 7.36 (br d, J = 7.6 Hz, 2H), 7.35-7.29 (m, |
| | | | 3H), 7.26-7.24 (m, 1H), 5.08 (s, 2H), 4.00 (d, J = |
| | | | 6.4 Hz, 1H), 3.83 (br d, J = 5.9 Hz, 1H), 3.64 (d, |
| | | | J = 9.2 Hz, 1H), 3.57-3.48 (m, 4H), 3.46-3.40 (m, |
| | | | 1H), 2.44 (br s, 1H), 2.41 (s, 3H), 2.39 (br s, 1H), |
| | | | 2.31-2.26 (m, 1H), 2.17 (br d, J = 8.0 Hz, 1H), 1.47 |
| | | | (s, 3H) |
| 345 | Similar to | m/z 581.1 | ¹H NMR (400 MHz, METHANOL-d₄) δ = 8.50 (s, |
| | Example 30 | [M + H]⁺ | 1H), 8.05-7.93 (m, 1H), 7.62-7.44 (m, 2H), 7.42- |
| | | | 7.29 (m, 4H), 7.28-7.19 (m, 2H), 6.66-6.46 (m, |
| | | | 1H), 4.59 (br s, 2H), 4.49-4.33 (m, 2H), 4.31-3.99 |
| | | | (m, 2H), 3.85 (br s, 1H), 3.70-3.52 (m, 6H), 3.48 |
| | | | (br s, 1H), 2.45-2.25 (m, 2H), 2.24-1.93 (m, 4H), |
| | | | 1.50-1.36 (m, 3H) |
| 346 | Similar to | m/z 571.3 | ¹H NMR (400 MHz, CHLOROFORM-d) δ = 7.68 |
| | Example 30 | [M + H]⁺ | (br s, 1H), 7.48-7.40 (m, 1H), 7.36 (br d, J = 7.6 |
| | | | Hz, 2H), 7.33-7.28 (m, 2H), 7.25 (br s, 2H), 5.05 |
| | | | (s, 2H), 4.57-4.42 (m, 1H), 3.62 (br d, J = 9.2 Hz, |
| | | | 1H), 3.56-3.47 (m, 2H), 3.42 (br s, 1H), 3.25 (br d, |
| | | | J = 7.6 Hz, 1H), 3.04 (br d, J = 8.8 Hz, 2H), 2.60 (br |
| | | | t, J = 7.6 Hz, 1H), 2.49-2.44 (m, 1H), 2.37 (br s, |
| | | | 2H), 2.27 (br d, J = 4 Hz, 2H), 2.13 (br s, 2H), 2.07 |
| | | | (br d, J = 4.0 Hz, 2H), 1.87-1.82 (m, 2H), 1.74 (br |
| | | | d, J = 9.6 Hz, 2H), 1.61 (br d, J = 8.4 Hz, 1H), |
| | | | 1.46 (br s, 3H) |
| 347 | Similar to | m/z 568.2 | ¹H NMR (400 MHz, CHLOROFORM-d) δ = 7.66 |
| | Example 30 | [M + H]⁺ | (br s, 2H), 7.49 (br d, J = 7.0 Hz, 1H), 7.41-7.33 |
| | | | (m, 2H), 7.33-7.29 (m, 2H), 7.26 (br d, J = 3.6 Hz, |
| | | | 2H), 6.63 (br d, J = 8.5 Hz, 1H), 5.07 (s, 2H), 4.36 |
| | | | (br dd, J = 7.0, 11.9 Hz, 1H), 4.12-3.99 (m, 2H), |
| | | | 3.87-3.75 (m, 3H), 3.62 (br d, J = 8.9 Hz, 1H), |
| | | | 3.59-3.47 (m, 4H), 3.46-3.40 (m, 1H), 3.18 (br d, J = |
| | | | 1.1 Hz, 2H), 2.42-2.35 (m, 1H), 2.31-2.23 (m, |
| | | | 1H), 1.46 (s, 3H) |
| 348 | Similar to | m/z 407.0 | ¹H NMR (CHLOROFORM-d, 400 MHz): δ = 7.64 |
| | Example 30 | [M + H]⁺ | (d, J = 2.0 Hz, 1.0H), 7.34-7.41 (m, 2.1H), 7.27-7.34 |
| | | | (m, 3.0H), 7.25 (s, 1.2H), 6.84 (br s, 1.0H), 4.73 (d, |
| | | | J = 6.4 Hz, 2.1H), 3.52-3.63 (m, 2.0H), 3.49 (d, J = 8.8 |
| | | | Hz, 1.2H), 3.42 (td, J = 8.8, 3.5 Hz, 1.0H), 3.08 (d, |
| | | | J = 4.8 Hz, 3.0H), 2.32-2.43 (m, 1.1H), 2.28 (br dd, |
| | | | J = 7.2, 3.7 Hz, 1.1H), 2.01 (s, 3.3H), 1.45 ppm (s, |
| | | | 3.1H) |
| 349 | Similar to | m/z 457.1 | ¹H NMR (METHANOL-d₄, 400 MHz): δ = 7.82 (d, |
| | Example 30 | [M + H]⁺ | J = 1.2 Hz, 1.0H), 7.44 (s, 1.0H), 7.30-7.39 (m, |
| | | | 4.0H), 7.22 (br d, J = 7.2 Hz, 1.0H), 4.59 (s, 2.0H), |
| | | | 3.51-3.61 (m, 3.2H), 3.37-3.44 (m, 1.1H), 3.13 (q, |
| | | | J = 7.2 Hz, 2.1H), 2.96 (s, 3.0H), 2.25-2.40 (m, |
| | | | 2.1H), 1.42 (s, 3.0H), 1.32 ppm (t, J = 7.2 Hz, 3.0H) |
| 350 | Similar to | m/z 424.2 | ¹H NMR (400 MHz, CHLOROFORM-d) δ = 7.81- |
| | Example 30 | [M + H]⁺ | 7.61 (m, 1H), 7.60-7.46 (m, 1H), 7.31-7.25 (m, |
| | | | 2H), 7.23-7.15 (m, 4H), 5.13-4.74 (m, 2H), 3.56- |
| | | | 3.26 (m, 6H), 2.29-2.22 (m, 1H), 2.20-2.13 (m, |
| | | | 1H), 1.35 (s, 3H), 1.22 (br s, 6H) |
| 351 | Similar to | m/z 366.0 | ¹H NMR (400 MHz, CHLOROFORM-d) δ = 7.60- |
| | Example 30 | [M + H]⁺ | 7.54 (m, 1H), 7.32-7.16 (m, 7H), 4.98 (s, 2H), |
| | | | 3.56-3.51 (m, 1H), 3.49-3.40 (m, 2H), 3.38-3.30 (m, |
| | | | 1H), 3.03-2.92 (m, 3H), 2.35-2.24 (m, 1H), |
| | | | 2.23-2.15 (m, 1H), 1.44-1.32 (m, 3H) |
| 352 | Similar to | m/z 422.2 | ¹H NMR (400 MHz, CHLOROFORM-d) δ = 7.69- |
| | Example 30 | [M + H]⁺ | 7.61 (m, 1H), 7.58-7.45 (m, 1H), 7.40-7.34 (m, |
| | | | 2H), 7.33-7.25 (m, 4H), 5.21-4.93 (m, 2H), 4.84- |
| | | | 4.68 (m, 1H), 4.08-3.98 (m, 1H), 3.95-3.79 (m, |
| | | | 3H), 3.65-3.58 (m, 1H), 3.57-3.47 (m, 2H), 3.46- |
| | | | 3.37 (m, 1H), 2.43-2.33 (m, 2H), 2.31-2.23 (m, |
| | | | 1H), 2.05-1.96 (m, 1H), 1.45 (s, 3H) |

### Example 31

### 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylic acid (Compound 31)

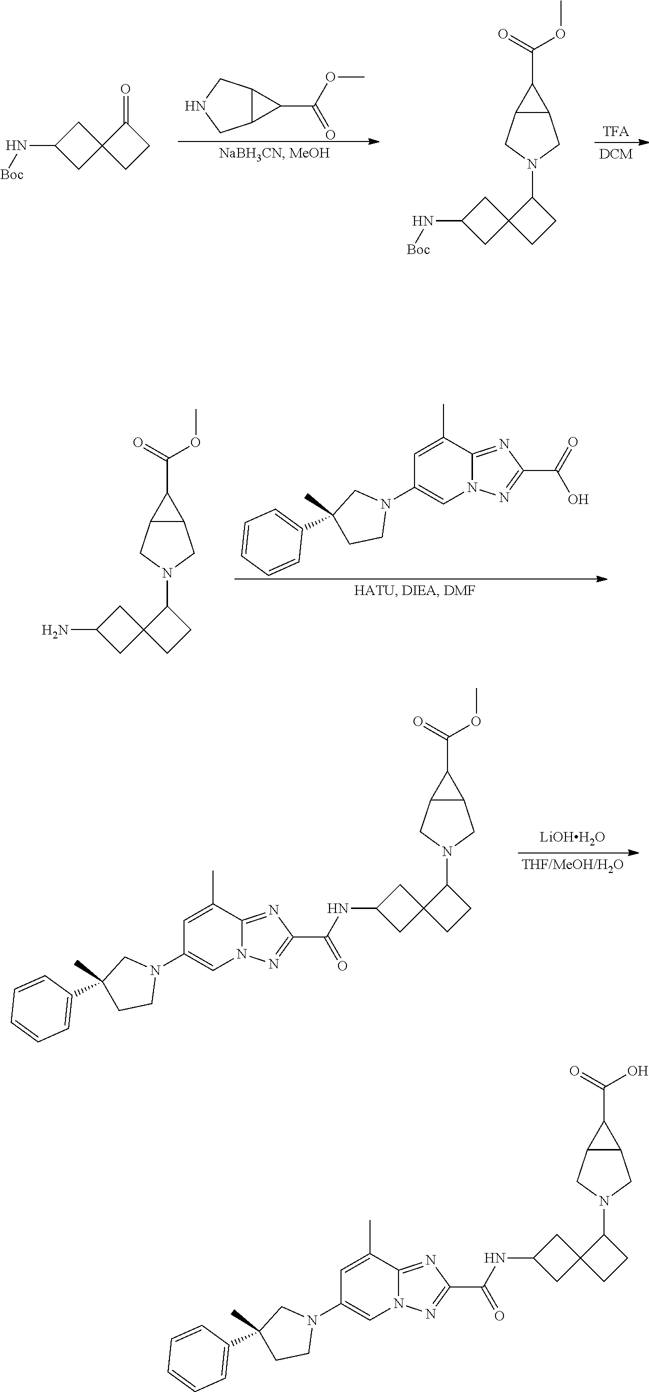

### Step 1: methyl 3-[2-(tert-butoxycarbonylamino)spiro[3.3]heptan-7-yl]-3-azabicyclo[3.1.0]hexane-6-carboxylate

A mixture of tert-butyl N-(7-oxospiro[3.3]heptan-2-yl)carbamate (100 mg, 443.89 μmol, 1 eq), methyl 3-azabicyclo[3.1.0]hexane-6-carboxylate (62.66 mg, 443.89 μmol, 1 eq), sodium;cyanoboranuide (41.84 mg, 665.83 μmol, 1.5 eq), AcOH (1.33 mg, 22.19 μmol, 1.27 μL, 0.05 eq) in MeOH (1 mL) was degassed and purged with N₂ for 3 times, and then the mixture was stirred at 25° C. for 2 h under N₂ atmosphere. LCMS showed starting material was consumed completely and the desired compound was detected. The reaction was concentrated under reduced pressure to give compound methyl 3-[2-(tert-butoxycarbonylamino) spiro[3.3]heptan-7-yl]-3-azabicyclo[3.1.0]hexane-6-carboxylate (150 mg, crude) was obtained as a yellow oil.

LC-MS [ESI, M+1]:351.0

### Step 2: methyl 3-(2-aminospiro[3.3]heptan-7-yl)-3-azabicyclo[3.1.0]hexane-6-carboxylate

To a solution of methyl 3-[2-(tert-butoxycarbonylamino) spiro[3.3]heptan-7-yl]-3-azabicyclo[3.1.0]hexane-6-carboxylate (150 mg, 428.02 μmol, 1 eq) methyl 3-[2-(tert-butoxycarbonylamino)spiro[3.3]heptan-7-yl]-3-azabicyclo[3.1.0]hexane-6-carboxylate (150 mg, 428.02 μmol, 1 eq) in DCM (2 mL) was added TFA (48.80 mg, 428.02 μmol, 31.79 μL, 1 eq). The mixture was stirred at 25 C for 2 h. LCMS showed starting material was consumed completely and the desired compound was detected. The reaction was concentrated under reduced pressure to give compound methyl 3-(2-aminospiro[3.3]heptan-7-yl)-3-azabicyclo[3.1.0]hexane-6-carboxylate (70 mg, 65% yield) was obtained as a white oil.

LC-MS [ESI, M+1]:250.9

### Step 3: methyl 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylate

To a solution of 8-methyl-6-[(3R)-3-methyl-3-phenyl-pyrrolidin-1-yl]-[1,2,4]triazolo[1,5-a]pyridine-2-carboxylic acid (50 mg, 148.64 μmol, 1 eq) in DMF (2 mL) was added HATU (113.03 mg, 297.28 μmol, 2 eq) and DIEA (57.63 mg, 445.92 μmol, 77.67 μL, 3 eq), methyl 3-(2-aminospiro[3.3]heptan-7-yl)-3-azabicyclo[3.1.0]hexane-6-carboxylate (37.21 mg, 102.12 μmol, 6.87e-1 eq, TFA). The mixture was stirred at 25° C. for 1 h. LCMS showed starting material was consumed completely and the desired compound was detected. The aqueous phase was extracted with EtOAc (10 mL×2).The combined organic phase was washed with brine (10 mL), dried with anhydrous Na2SO4, filtered and concentrated in vacuum. The residue was purified by prep-HPLC (column: Phenomenex luna C18 150*25 mm*10 um; mobile phase: [water(FA)—ACN]; gradient:25%-55% B over 10 min) and lyophilized to give compound methyl 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylate.

LC-MS [ESI, M+1]:569.4

### Step 4: 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylic acid

To a solution of methyl 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylate (20 mg, 35.17 μmol, 1 eq) in THF (0.5 mL) was added LiOH·H₂O (2.95 mg, 70.33 μmol, 2 eq). The mixture was stirred at 25° C. for 2 h. LCMS showed starting material was consumed completely and the desired compound was detected. The reaction was concentrated under reduced pressure to give a residue. The residue was purified by prep-HPLC (column: henomenex luna C18 150*25 mm*10 um; mobile phase: [water(FA)—ACN]; gradient:16%-46% B over 15 min) and lyophilizated to give compound 3-[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]-3-azabicyclo[3.1.0]hexane-6-carboxylic acid (2.86 mg, 15% yield) was obtained as a white solid.

LC-MS [ESI, M+1]: 555.1.

¹H NMR (400 MHz, CHLOROFORM-d) δ=7.6 (s, 1H), 7.51 (d, J=7.6 Hz, 1H), 7.40-7.30 (m, 4H), 7.24 (s, 1H), 7.00 (s, 1H), 4.57-4.47 (m, 1H), 3.60 (d, J=8.8 Hz, 1H), 3.55-3.46 (m, 2H), 3.45-3.36 (m, 1H), 3.26 (d, J=8.4 Hz, 1H), 3.10-3.01 (m, 2H), 2.66-2.57 (m, 5H), 2.51-2.46 (m, 2H), 2.37 (d, J=3.2 Hz, 2H), 2.29-2.25 (m, 1H), 2.12 (s, 1H), 2.07 (s, 1H), 2.01 (s, 1H), 1.86 (dd, J=6.4, 16.6 Hz, 2H), 1.73 (dd, J=8.8, 14.3 Hz, 2H), 1.65-1.57 (m, 1H), 1.47 (s, 3H).

| Cmpd. No. | Synthesis | LC/MS | ¹H NMR |
|---|---|---|---|
| 353 | Similar to | m/z 585.3 | ¹H NMR (400 MHz, CHLOROFORM-d) δ = 7.72- |
| | Example 31 | [M + H]⁺ | 7.60 (m, 1H), 7.43-7.34 (m, 2H), 7.34-7.29 (m, 2H), |
| | | | 7.26-7.22 (m, 1H), 7.00 (br s, 1H), 4.47-4.10 (m, 2H), |
| | | | 3.99-3.83 (m, 2H), 3.82-3.73 (m, 1H), 3.68 (br d, J = |
| | | | 8.1 Hz, 1H), 3.63-3.57 (m, 1H), 3.56-3.31 (m, 4H), |
| | | | 3.28-3.11 (m, 2H), 2.91-2.78 (m, 2H), 2.63 (br d, J = |
| | | | 8.8 Hz, 3H), 2.54-2.50 (m, 1H), 2.43-2.33 (m, 2H), |
| | | | 2.31-2.20 (m, 2H), 1.98 (br s, 2H), 1.93-1.87 (m, 1H), |
| | | | 1.80-1.71 (m, 1H), 1.62-1.53 (m, 1H), 1.46 (s, 3H) |
| 354 | Similar to | m/z 515.2 | ¹H NMR (CHLOROFORM-d, 400 MHz): δ = 7.94- |
| | Example 31 | [M + H]⁺ | 8.18 (m, 1.0H), 7.58 (br s, 1.0H), 7.32 (br d, J = 7.2 Hz, |
| | | | 2.0H), 7.27-7.29 (m, 2.0H), 7.20-7.25 (m, 1.0H), 6.91 |
| | | | (br s, 0.9H), 4.30-4.47 (m, 1.0H), 3.51 (br d, J = 8.4 Hz, |
| | | | 1.0H), 3.37-3.47 (m, 2.0H), 3.33 (br s, 1.1H), 3.10 (br |
| | | | d, J = 8.0 Hz, 2.0H), 2.91 (br s, 1.1H), 2.40-2.58 (m, |
| | | | 7.1H), 2.30 (br s, 1.2H), 2.07-2.24 (m, 3.1H), 1.99 (br |
| | | | s, 3.0H), 1.41 ppm (s, 3.0H) |
| 355 | Similar to | m/z 585.3 | ¹H NMR (DMSO-d₆, 400 MHz): δ = 8.29 (s, 0.5H), |
| | Example 31 | [M + H]⁺ | 7.89 (br d, J = 18.4 Hz, 1.0H), 7.33-7.45 (m, 4.0H), |
| | | | 7.25-7.33 (m, 0.9H), 7.22-7.25 (m, 1.1H), 3.83 (br dd, |
| | | | J = 10.4, 2.6 Hz, 2.0H), 3.57-3.70 (m, 3.0H), 3.42-3.54 |
| | | | (m, 4.1H), 3.39 (br dd, J = 13.2, 7.1 Hz, 2.2H), 3.21-3.29 |
| | | | (m, 1.0H), 3.00-3.14 (m, 1.0H), 2.91-2.99 (m, 1.0H), |
| | | | 2.77-2.84 (m, 1.0H), 2.53 (br d, J = 4.0 Hz, 3.1H), |
| | | | 2.31-2.39 (m, 1.1H), 2.16-2.28 (m, 2.1H), 2.00-2.14 (m, |
| | | | 1.1H), 1.84 (brs, 1.0H), 1.75-1.82 (m, 1.0H), 1.70-1.74 |
| | | | (m, 1.2H), 1.66 (br d, J = 2.4 Hz, 1.0H), 1.56-1.64 (m, |
| | | | 2.0H), 1.43-1.52 (m, 1.1H), 1.36 ppm (br s, 3.0H) |
| 356 | Similar to | m/z 569.6 | ¹H NMR (400 MHz, CHLOROFORM-d) δ = 7.64 (br |
| | Example 31 | [M + H]⁺ | s, 1H), 7.60-7.52 (m, 1H), 7.37 (br d, J = 7.2 Hz, 2H), |
| | | | 7.34-7.30 (m, 2H), 7.27 (br s, 1H), 7.00 (br s, 1H), |
| | | | 4.61-4.45 (m, 1H), 3.81-3.56 (m, 3H), 3.55-3.36 (m, |
| | | | 4H), 3.03-2.83 (m, 2H), 2.62 (br s, 3H), 2.53-2.43 |
| | | | (m, 3H), 2.41-2.35 (m, 2H), 2.26 (br s, 2H), 2.15 (br |
| | | | d, J = 7.2 Hz, 4H), 1.60 (br s, 4H), 1.46 (s, 3H) |
| 357 | Similar to | m/z 583.6 | ¹H NMR (400 MHz, CHLOROFORM-d) δ = 8.45 (s, |
| | Example 31 | [M + H]⁺ | 1H), 7.65 (s, 1H), 7.42-7.36 (m, 2H), 7.36-7.28 (m, |
| | | | 3H), 7.25 (br s, 1H), 6.99 (s, 1H), 4.58 (br d, J = 8.0 |
| | | | Hz, 1H), 3.68 (s, 3H), 3.60 (d, J = 9.2 Hz, 1H), 3.56- |
| | | | 3.45 (m, 2H), 3.41 (dt, J = 3.6, 8.8 Hz, 1H), 3.17 (br d, |
| | | | J = 9.6 Hz, 2H), 3.07 (br t, J = 8.0 Hz, 1H), 2.63 |
| | | | (s, 3H), 2.60-2.48 (m, 3H), 2.48-2.41 (m, 1H), 2.41- |
| | | | 2.34 (m, 1H), 2.28-2.13 (m, 6H), 2.09-2.00 (m, 4H), |
| | | | 1.47 (s, 3H), 1.38 (br s, 2H) |
| 358 | Similar to | m/z 559.2 | ¹H NMR (400 MHz, CHLOROFORM-d) δ = 7.69 (s, |
| | Example 31 | [M + H]⁺ | 2H), 7.47 (br d, J = 7.6 Hz, 1H), 7.42-7.31 (m, 5H), |
| | | | 7.02 (s, 1H), 6.63 (br d, J = 8.4 Hz, 1H), 4.68 (br d, |
| | | | J = 7.2 Hz, 1H), 3.66-3.49 (m, 6H), 3.46-3.40 (m, 1H), |
| | | | 2.65 (s, 3H), 2.46-2.24 (m, 5H), 1.49 (s, 3H) |
| 359 | Similar to | m/z 559.2 | ¹H NMR (400 MHz, CHLOROFORM-d) δ = 7.66 (d, J = |
| | Example 31 | [M + H]⁺ | 1.6 Hz, 1H), 7.61 (br d, J = 8.4 Hz, 1H), 7.41-7.34 |
| | | | (m, 2H), 7.34-7.30 (m, 2H), 7.27-7.24 (m, 1H), 7.01 |
| | | | (s, 1H), 4.39 (q, J = 8.0 Hz, 3H), 3.61 (d, J = 8.8 Hz, |
| | | | 1H), 3.57-3.46 (m, 2H), 3.41 (dt, J = 3.6, 8.8 Hz, 1H), |
| | | | 3.34 (br s, 1H), 3.26 (br s, 1H), 2.95-2.85 (m, 1H), |
| | | | 2.80 (br d, J = 8.8 Hz, 1H), 2.68 (br d, J = 6.4 Hz, 1H), |
| | | | 2.63 (s, 3H), 2.48-2.34 (m, 3H), 2.32-2.22 (m, 2H), |
| | | | 2.01 (br d, J = 5.2 Hz, 4H), 1.89 (br t, J = 9.6 Hz, 2H), |
| | | | 1.47 (s, 3H) |
| 360 | Similar to | m/z 573.3 | ¹H NMR (400 MHz, CHLOROFORM-d) δ = 7.65 (d, J = |
| | Example 31 | [M + H]⁺ | 1.2 Hz, 1H), 7.53 (br d, J = 8.4 Hz, 1H), 7.40-7.29 |
| | | | (m, 4H), 7.27-7.23 (m, 1H), 7.00 (s, 1H), 4.46-4.27 |
| | | | (m, 3H), 3.71 (s, 3H), 3.60 (d, J = 8.8 Hz, 1H), 3.57- |
| | | | 3.46 (m, 2H), 3.41 (dt, J = 3.6, 8.8 Hz, 1H), 3.37-3.28 |
| | | | (m, 1H), 3.23 (br t, J = 6.8 Hz, 1H), 2.94-2.84 (m, 1H), |
| | | | 2.76 (br d, J = 10.8 Hz, 1H), 2.63 (s, 4H), 2.45-2.31 |
| | | | (m, 3H), 2.31-2.18 (m, 2H), 2.00-1.90 (m, 4H), 1.82 |
| | | | (br d, J = 11.6 Hz, 2H), 1.47 (s, 3H) |
| 361 | Similar to | m/z 491.3 | ¹H NMR (400 MHz, DMSO-d₆) δ = 8.66 (d, J = 8.0 |
| | Example 31 | [M + H]⁺ | Hz, 1H), 7.87 (s, 1H), 7.44-7.32 (m, 4H), 7.30 (s, 1H), |
| | | | 7.27-7.20 (m, 1H), 4.83 (br d, J = 4.0 Hz, 2H), 4.21- |
| | | | 4.05 (m, 1H), 3.83 (br d, J = 4.0 Hz, 2H), 3.64-3.55 |
| | | | (m, 1H), 3.54-3.46 (m, 2H), 3.39 (br s, 1H), 2.79- |
| | | | 2.67 (m, 3H), 2.55 (s, 3H), 2.34-2.22 (m, 6H), 2.09- |
| | | | 1.97 (m, 2H), 1.35 (s, 3H) |
| 362 | Similar to | m/z 491.2 | ¹H NMR (400 MHz, DMSO-d₆) δ = 8.67 (d, J = 8.0 |
| | Example 31 | [M + H]⁺ | Hz, 1H), 7.87 (s, 1H), 7.42-7.32 (m, 4H), 7.31 (s, 1H), |
| | | | 7.27-7.20 (m, 1H), 4.82 (d, J = 4.4 Hz, 1H), 4.52 (br |
| | | | d, J = 4.8 Hz, 2H), 4.11 (sxt, J = 8.4 Hz, 1H), |
| | | | 3.99-3.80 (m, 2H), 3.63-3.56 (m, 1H), 3.55-3.45 (m, 2H), |
| | | | 3.41-3.36 (m, 1H), 2.88-2.66 (m, 3H), 2.56 (s, 3H), |
| | | | 2.35-2.21 (m, 6H), 2.08-1.97 (m, 2H), 1.36 (s, 3H) |
| 363 | Similar to | m/z 535.4 | ¹H NMR (400 MHz, CHLOROFORM-d) δ = 7.66 (s, |
| | Example 31 | [M + H]⁺ | 1H), 7.41-7.34 (m, 3H), 7.34-7.30 (m, 2H), 7.25 (br |
| | | | s, 1H), 7.00 (s, 1H), 5.25-5.14 (m, 1H), 5.12-5.01 (m, |
| | | | 1H), 4.67-4.55 (m, 1H), 3.61 (d, J = 8.8 Hz, 1H), 3.56- |
| | | | 3.46 (m, 2H), 3.41 (dt, J = 3.6, 8.5 Hz, 1H), 3.07- |
| | | | 2.92 (m, 3H), 2.83-2.67 (m, 2H), 2.63 (s, 3H), 2.50- |
| | | | 2.42 (m, 1H), 2.41-2.34 (m, 1H), 2.31-2.23 (m, 2H), |
| | | | 2.16-1.97 (m, 6H), 1.47 (s, 3H) |
| 364 | Similar to | m/z 555.4 | ¹H NMR (400 MHz, CHLOROFORM-d) δ = 7.64 (br |
| | Example 31 | [M + H]+ | s, 1H), 7.46 (br d, J = 4.4 Hz, 1H), 7.40-7.33 (m, 2H), |
| | | | 7.33-7.28 (m, 2H), 7.26-7.22 (m, 1H), 6.99 (br s, 1H), |
| | | | 4.65-4.48 (m, 1H), 3.59 (br d, J = 8.4 Hz, 1H), 3.52- |
| | | | 3.45 (m, 2H), 3.39 (br s, 2H), 3.29 (br d, J = 6.4 Hz, |
| | | | 2H), 3.16 (br dd, J = 2.8, 3.9 Hz, 2H), 2.70-2.53 (m, |
| | | | 6H), 2.50-2.29 (m, 3H), 2.25-2.13 (m, 3H), 2.07 (br |
| | | | s, 4H), 1.45 (s, 3H) |
| 365 | Similar to | m/z 529.3 | ¹H NMR (400 MHz, CHLOROFORM-d) δ = 8.06 (br |
| | Example 31 | [M + H]+ | d, J = 8.4 Hz, 1H), 7.79 (s, 1H), 7.64 (s, 1H), 7.42- |
| | | | 7.33 (m, 2H), 7.33-7.28 (m, 2H), 7.24 (s, 1H), 7.02- |
| | | | 6.92 (m, 1H), 4.73-4.48 (m, 1H), 3.59 (br d, J = 8.8 |
| | | | Hz, 1H), 3.53-3.33 (m, 5H), 2.93-2.88 (m, 1H), 2.72- |
| | | | 2.55 (m, 5H), 2.43-2.29 (m, 2H), 2.28-2.18 (m, 1H), |
| | | | 2.16-2.00 (m, 4H), 1.97-1.77 (m, 3H), 1.75-1.57 (m, |
| | | | 1H), 1.51-1.37 (m, 3H) |
| 366 | Similar | m/ m/z | ¹H NMR (400 MHz, DMSO-d₆) δ = 7.90 (s, 1H), 7.43- |
| | to | 529.2 | 7.32 (m, 4H), 7.29 (s, 1H), 7.24 (br d, J = 7.2 Hz, 1H), |
| | Example 31 | [M + H]+ | 4.16 (br d, J = 12.8 Hz, 1H), 3.84-3.76 (m, 1H), 3.61- |
| | | | 3.57 (m, 1H), 3.52 (br d, J = 9.2 Hz, 3H), 3.16 (br d, |
| | | | J = 10.8 Hz, 2H), 3.06 (br d, J = 8.8 Hz, 1H), 3.01 (br |
| | | | d, J = 8.9 Hz, 1H), 2.51 (br s, 3H), 2.37 (br t, J = 9.6 |
| | | | Hz, 3H), 2.25 (br t, J = 6.8 Hz, 2H), 1.84 (br s, 3H), |
| | | | 1.70 (br s, 2H), 1.42-1.27 (m, 5H) |
| 367 | Similar to | m/z 554.3 | ¹H NMR (400 MHz, DMSO-d₆) δ = 8.63 (br d, J = 8.0 |
| | Example 31 | [M + H]+ | Hz, 1H), 7.86 (s, 1H), 7.51 (br s, 1H), 7.42-7.29 (m, |
| | | | 5H), 7.27-7.20 (m, 1H), 7.04-6.67 (m, 1H), 4.42- |
| | | | 4.26 (m, 1H), 3.62-3.57 (m, 1H), 3.54-3.46 (m, 2H), |
| | | | 3.42-3.37 (m, 1H), 3.36 (br s, 3H), 3.19-2.77 (m, 2H), |
| | | | 2.55 (s, 3H), 2.41-2.11 (m, 8H), 2.10-1.88 (m, 3H), |
| | | | 1.87-1.71 (m, 2H), 1.36 (s, 3H) |
| 368 | Similar to | m/z 579.3 | ¹H NMR (400 MHz, DMSO-d₆) δ = 8.61 (d, J = 7.6 |
| | Example 31 | [M + H]+ | Hz, 1H), 7.87 (s, 1H), 7.42-7.30 (m, 5H), 7.27-7.20 |
| | | | (m, 1H), 4.40-4.26 (m, 1H), 3.64-3.56 (m, 1H), 3.55- |
| | | | 3.45 (m, 2H), 3.42-3.38 (m, 1H), 3.08-2.97 (m, 3H), |
| | | | 2.55 (s, 3H), 2.43-2.35 (m, 3H), 2.34-2.31 (m, 1H), |
| | | | 2.29-2.23 (m, 2H), 2.19 (br t, J = 9.2 Hz, 3H), |
| | | | 2.03-1.96 (m, 3H), 1.95-1.82 (m, 2H), 1.36 (s, 3H) |
| 369 | Similar to | m/z 531.4 | ¹H NMR (400 MHz, CHLOROFORM-d) δ = 8.60- |
| | Example 31 | [M + H]+ | 8.39 (m, 1H), 7.62 (br s, 1H), 7.48 (br d, J = 7.2 Hz, |
| | | | 1H), 7.40-7.33 (m, 2H), 7.33-7.28 (m, 2H), 7.27- |
| | | | 7.22 (m, 1H), 6.99 (br s, 1H), 4.66-4.51 (m, 1H), 4.38 |
| | | | (br s, 2H), 3.58 (br d, J = 8.8 Hz, 1H), 3.53-3.45 (m, |
| | | | 2H), 3.39 (br s, 2H), 3.26-2.98 (m, 4H), 2.61 (s, 3H), |
| | | | 2.59-2.47 (m, 2H), 2.46-2.29 (m, 4H), 2.28-2.20 (m, |
| | | | 2H), 2.19-2.09 (m, 2H), 1.45 (s, 3H) |
| 370 | Similar to | m/z 585.3 | ¹H NMR (400 MHz, CHLOROFORM-d) δ = 7.64 (br |
| | Example 31 | [M + H]+ | s, 1H), 7.41-7.34 (m, 2H), 7.33-7.29 (m, 2H), 7.27 |
| | | | (s, 1H), 6.99 (br s, 1H), 4.38-4.22 (m, 1H), 4.02 (br d, |
| | | | J = 9.2 Hz, 1H), 3.90 (br d, J = 6.0 Hz, 1H), 3.60 (br d, |
| | | | J = 8.4 Hz, 2H), 3.55-3.45 (m, 3H), 3.42 (br d, J = 8.4 |
| | | | Hz, 3H), 3.17-2.97 (m, 4H), 2.63 (br s, 3H), 2.51- |
| | | | 2.31 (m, 3H), 2.29-2.19 (m, 1H), 1.97 (br s, 1H), |
| | | | 1.95-1.60 (m, 6H), 1.46 (s, 3H) |
| 371 | Similar to | m/z 543.3 | ¹H NMR (400 MHz, CHLOROFORM-d) δ ppm 1.18- |
| | Example 31 | [M + H]⁺ | 1.52 (m, 6 H) 1.99-2.30 (m, 4 H) 2.31-2.58 (m, 5 H) |
| | | | 2.76 (br s, 4 H) 3.22-3.55 (m, 4 H) 3.75-4.32 (m, 5 |
| | | | H) 4.41-4.54 (m, 1 H) 6.84-6.98 (m, 1 H) 7.15-7.27 |
| | | | (m, 3 H) 7.27-7.35 (m, 2 H) 7.42-7.62 (m, 1 H) 7.99 |
| | | | (br s, 1 H) |
| 372 | Similar | m/z 569.1 | ¹H NMR (400 MHz, CHLOROFORM-d) δ = 7.64 (s, |
| | to | [M + H]+ | 1H), 7.56-7.43 (m, 1H), 7.39-7.35 (m, 2H), 7.34- |
| | Example 31 | | 7.30 (m, 2H), 7.26-7.22 (m, 1H), 7.01 (s, 1H), 4.57- |
| | | | 4.46 (m, 1H), 3.71-3.64 (m, 3H), 3.6 (br d, J = 9.0 Hz, |
| | | | 1H), 3.56-3.46 (m, 2H), 3.45-3.34 (m, 2H), 3.18- |
| | | | 3.08 (m, 1H), 2.68-2.52 (m, 5H), 2.44-2.21 (m, 3H), |
| | | | 2.17-2.01 (m, 4H), 1.95-1.87 (m, 2H), 1.78 (br d, |
| | | | J = 8.8 Hz, 4H), 1.47 (s, 3H) |
| 373 | Similar to | m/z 571.3 | ¹H NMR (400 MHz, CHLOROFORM-d) δ = 7.66 (s, |
| | Example 31 | [M + H]⁺ | 1H), 7.51 (br d, J = 7.2 Hz, 1H), 7.41-7.34 (m, 2H), |
| | | | 7.34-7.29 (m, 2H), 7.25 (br s, 1H), 7.00 (s, 1H), 4.64- |
| | | | 4.47 (m, 1H), 3.72 (s, 3H), 3.61 (br d, J = 8.9 Hz, 1H), |
| | | | 3.57-3.45 (m, 2H), 3.45-3.37 (m, 1H), 3.35-3.15 (m, |
| | | | 1H), 3.07-2.78 (m, 2H), 2.64 (s, 4H), 2.53-2.33 (m, |
| | | | 3H), 2.31-2.19 (m, 2H), 2.17-1.75 (m, 11H), 1.47 (s, |
| | | | 3H) |
| 374 | Similar to | m/z 557.3 | ¹H NMR (400 MHz, CHLOROFORM-d) δ = 8.20 (br |
| | Example 31 | [M + H]⁺ | d, J = 7.2 Hz, 1H), 7.60 (br s, 1H), 7.41-7.27 (m, 4H), |
| | | | 7.26-7.19 (m, 1H), 6.94 (br s, 1H), 4.74-4.45 (m, 1H), |
| | | | 3.59-3.31 (m, 5H), 3.28-3.16 (m, 1H), 2.96-2.71 (m, |
| | | | 2H), 2.56 (s, 3H), 2.51-1.88 (m, 15H), 1.76 (br d, J = |
| | | | 9.6 Hz, 1H), 1.44 (s, 3H) |
| 375 | Similar to | m/z 557.2 | ¹H NMR (400 MHz, CHLOROFORM-d) δ = 7.66 (br |
| | Example 31 | [M + H]⁺ | s, 1H), 7.48-7.36 (m, 2H), 7.34-7.31 (m, 2H), 7.25 |
| | | | (br s, 1H), 7.00 (br s, 1H), 4.42-4.33 (m, 2H), 4.28 (br |
| | | | d, J = 12.0 Hz, 1H), 4.06-4.00 (m, 1H), 4.00-3.92 (m, |
| | | | 1H), 3.82-3.68 (m, 1H), 3.54 (br d, J = 8.0 Hz, 1H), |
| | | | 3.49 (br d, J = 8.8 Hz, 1H), 3.66-3.36 (m, 3H), 3.09- |
| | | | 2.94 (m, 2H), 2.64 (br s, 3H), 2.36 (br dd, J = 8.8, 14.0 |
| | | | Hz, 3H), 2.31-2.18 (m, 4H), 2.02 (br d, J = 3.6 Hz, |
| | | | 3H), 1.47 (s, 3H) |
| 376 | Similar to | m/z 571.4 | ¹H NMR (400 MHz, CHLOROFORM-d) δ = 8.43 (br |
| | Example 31 | [M + H]⁺ | s, 1H), 7.65 (br s, 1H), 7.40-7.34 (m, 2H), 7.33-7.29 |
| | | | (m, 2H), 7.27-7.22 (m, 1H), 7.00 (s, 1H), 4.19-3.97 |
| | | | (m, 2H), 3.87-3.72 (m, 2H), 3.60 (br d, J = 8.9 Hz, |
| | | | 2H), 3.57-3.46 (m, 2H), 3.41 (dt, J = 3.5, 8.6 Hz, 1H), |
| | | | 3.31-3.19 (m, 1H), 3.18-3.01 (m, 1H), 3.00-2.68 (m, |
| | | | 2H), 2.63 (s, 3H), 2.58-2.40 (m, 1H), 2.39 (br d, J = |
| | | | 3.8 Hz, 1H), 2.31-2.22 (m, 2H), 2.21-1.98 (m, 6H), |
| | | | 1.97-1.66 (m, 2H), 1.65-1.55 (m, 1H), 1.54-1.27 (m, |
| | | | 5H) |
| 377 | Similar to | m/z 571.3 | ¹H NMR (400 MHz, CHLOROFORM-d) δ = 7.63 (br |
| | Example 31 | [M + H]+ | s, 1H), 7.36 (br d, J = 7.2 Hz, 2H), 7.33-7.29 (m, 2H), |
| | | | 7.27-7.21 (m, 1H), 6.99 (br s, 1H), 4.19-3.93 (m, 2H), |
| | | | 3.85-3.71 (m, 2H), 3.67-3.46 (m, 4H), 3.41 (dt, J = |
| | | | 3.2, 8.6 Hz, 2H), 3.30-3.10 (m, 2H), 3.06-2.82 (m, |
| | | | 2H), 2.63 (s, 3H), 2.53-1.94 (m, 10H), 1.92-1.60 (m, |
| | | | 3H), 1.57-1.29 (m, 5H) |
| 378 | Similar to | m/z 583.4 | ¹H NMR (400 MHz, CHLOROFORM-d) δ = 8.40 (br |
| | Example 31 | [M + H]+ | s, 1H), 7.58 (s, 1H), 7.43 (br d, J = 8.0 Hz, 1H), 7.35- |
| | | | 7.28 (m, 2H), 7.27-7.17 (m, 3H), 6.95 (s, 1H), 4.54- |
| | | | 4.36 (m, 1H), 4.04 (q, J = 7.2 Hz, 2H), 3.55 (br d, J = |
| | | | 8.8 Hz, 2H), 3.49-3.43 (m, 2H), 3.42-3.30 (m, 5H), |
| | | | 3.10 (br t, J = 8.0 Hz, 1H), 2.69-2.50 (m, 5H), 2.37- |
| | | | 2.28 (m, 1H), 2.25-2.13 (m, 3H), 2.06-1.99 (m, 2H), |
| | | | 1.80 (br s, 2H), 1.64 (br dd, J = 2.4, 14.2 Hz, 2H), |
| | | | 1.45-1.37 (m, 3H), 1.19 (t, J = 7.2 Hz, 3H) |
| 379 | Similar to | m/z 555.3 | ¹H NMR (400 MHz, DMSO-d₆) δ = 8.66 (d, J = 8.0 Hz, |
| | Example 31 | [M + H]+ | 1H), 7.87 (s, 1H), 7.42-7.35 (m, 3H), 7.32 (br d, J = |
| | | | 8.4 Hz, 2H), 7.26-7.20 (m, 1H), 4.37-4.24 (m, 1H), |
| | | | 3.62-3.57 (m, 1H), 3.52 (br d, J = 9.2 Hz, 2H), 3.37 |
| | | | (br dd, J = 3.4, 5.6 Hz, 1H), 3.02 (s, 2H), 2.90 (s, 2H), |
| | | | 2.68 (brt, J = 7.2 Hz, 1H), 2.55 (s, 3H), 2.34-2.21 (m, |
| | | | 6H), 2.04 (t, J = 2.8 Hz, 1H), 1.92-1.74 (m, 2H), 1.67 |
| | | | (br s, 2H), 1.59-1.50 (m, 2H), 1.35 (s, 3H) |
| 380 | Similar to | m/z 557.5 | ¹H NMR (400 MHz, CHLOROFORM-d) δ = 7.67- |
| | Example 31 | [M + H]+ | 7.47 (m, 2H), 7.35 (br d, J = 7.4 Hz, 2H), 7.33-7.28 |
| | | | (m, 2H), 7.24 (br s, 1H), 6.99 (br s, 1H), 4.65-4.56 (m, |
| | | | 1H), 3.65-3.33 (m, 6H), 3.16 (br dd, J = 2.0, 4.0 Hz, |
| | | | 2H), 2.87 (br d, J = 3.0 Hz, 2H), 2.61 (br s, 4H), 2.53- |
| | | | 2.34 (m, 5H), 2.30-2.21 (m, 2H), 2.19-2.08 (m, 2H), |
| | | | 1.90 (br d, J = 1.2 Hz, 2H), 1.45 (s, 3H) |

### Example 32

### 6-[(3R)-3-(3-fluorophenyl)-3-methyl-pyrrolidin-1l-yl]-N, 8-dimethyl-[1,2,4]triazolo-a]pyridine-2-carboxamide (Compound 32)

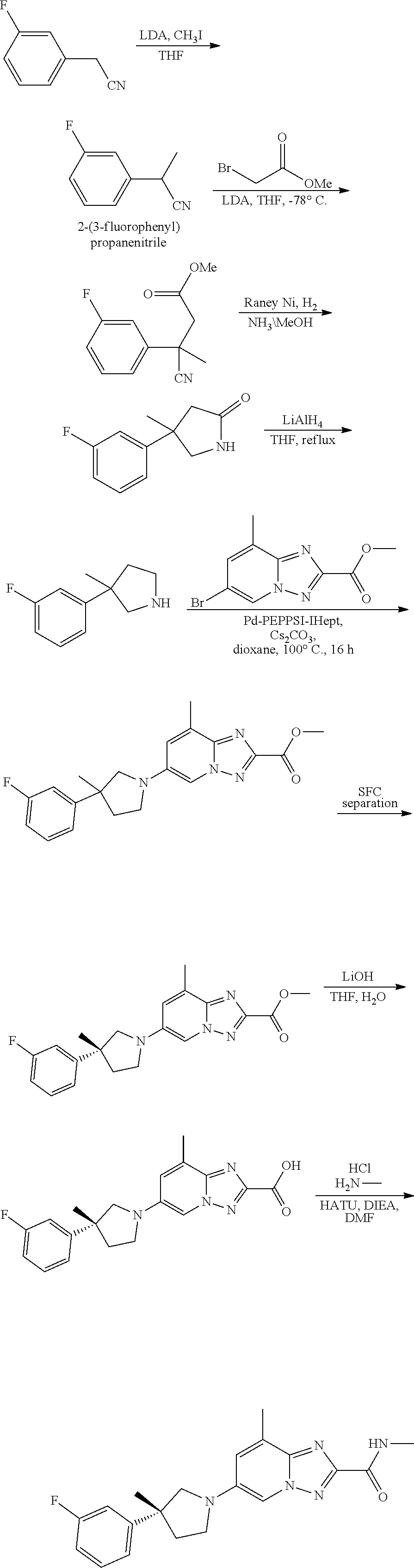

### Step 1: 2-(3-fluorophenyl)propanenitrile

To a solution of 2-(3-fluorophenyl)acetonitrile (20 g, 148.00 mmol, 17.24 mL, 1 eq) in THF (200 mL) was added dropwise LDA (2 M, 88.80 mL, 1.2 eq) −65° C. over 0.15 h. After addition, the mixture was stirred at this temperature for 0.35 h, and CH₃I (42.01 g, 295.99 mmol, 18.43 mL, 2 eq) was added dropwise at −65° C. The resulting mixture was stirred at −65° C. for 1 h. TLC (Rf=0.5, PE:EtOAc=5:1) indicated reactant was consumed completely and many new spot was formed. The cold reaction mixture was poured into NH₄Cl (100 ml). The reaction mixture was extracted with EtOAc (100 mL×3), the combined organic layers were washed with brine (200 mL×3), dried over anhydrous Na2SO4, filtered and dried to give a residue. The residue was purified by flash silica gel chromatography (SiO₂; PE:EtOAc=5:1, Rf=0.5) to give 2-(3-fluorophenyl)propanenitrile (10.5 g, 48% yield) as a yellow liquid.

¹H NMR (400 MHz, CHLOROFORM-d) δ=7.37 (dt, J=6.0, 7.8 Hz, 1H), 7.16 (br d, J=7.6 Hz, 1H), 7.11-6.99 (m, 2H), 3.91 (q, J=7.2 Hz, 1H), 2.04 (s, 1H), 1.65 (d, J=7.2 Hz, 3H), 1.26 (t, J=7.2 Hz, 1H)

### Step 2: methyl 3-cyano-3-(3-fluorophenyl)butanoate

To a solution of 2-(3-fluorophenyl)propanenitrile (10.5 g, 70.39 mmol, 1 eq) in THF (120 mL) was added LDA (2 M, 42.24 mL, 1.2 eq) at −65° C. the mixture was stirred at −65° C. for 1 h, then methyl 2-bromoacetate (11.84 g, 77.43 mmol, 7.31 mL, 1.1 eq) was added to the solution at −65° C., then the mixture was stirred at −65° C. for 1 h. TLC (Rf=0.3, PE:EtOAc=5:1) indicated reactant was consumed and many new spots formed. The cold reaction mixture was poured into NH₄Cl (50 ml). The reaction mixture was extracted with EtOAc (60 mL×3), the combined organic layers were washed with brine (80 mL×3), dried over anhydrous Na2SO₄, filtered and dried to give a residue. The residue was purified by flash silica gel chromatography (SiO₂; PE:EtOAc=5:1, Rf=0.3) to give compound methyl 3-cyano-3-(3-fluorophenyl)butanoate (10.5 g, 74% yield) as a yellow liquid

¹H NMR (400 MHz, CHLOROFORM-d) δ=7.42-7.34 (m, 1H), 7.29 (br d, J=8.0 Hz, 1H), 7.19 (td, J=2.0, 10.0 Hz, 1H), 7.04 (dt, J=2.0, 8.0 Hz, 1H), 3.66 (s, 3H), 2.97 (s, 2H), 1.83 (s, 3H)

### Step 3: 4-(3-fluorophenyl)-4-methyl-pyrrolidin-2-one

A solution of methyl 3-cyano-3-(3-fluorophenyl)butanoate (11 g, 49.72 mmol, 1 eq), NH₃·MeOH (7 M, 20.00 mL, 2.82 eq) in MeOH (200 mL) saturated with Raney-Ni (852.00 mg, 9.94 mmol, 0.2 eq), H2 (50 Psi) was stirred under 50 Psi at 50° C. for 48 h. LCMS showed starting material was consumed and the desired mass was detected. The reaction solution was filtered and the filtrate was concentrated under the reduced pressure to give the residue. The residue was purified by flash silica gel chromatography (SiO₂; PE:EtOAc=0:1, Rf=0.5) to give compound 4-(3-fluorophenyl)-4-methyl-pyrrolidin-2-one (7.2 g, 75% yield) as a white solid.

LC-MS [ESI, M+1]: 194.3.

¹H NMR (400 MHz, CHLOROFORM-d) δ=7.36-7.27 (m, 1H), 7.09-6.81 (m, 3H), 3.66 (br d, J=9.6 Hz, 1H), 3.48 (br d, J=9.6 Hz, 1H), 2.76 (br d, J=16.4 Hz, 1H), 2.51-2.36 (m, 1H), 1.57-1.44 (m, 3H).

### Step 4: 3-(3-fluorophenyl)-3-methyl-pyrrolidine

To a solution of LiAlH₄ (4.81 g, 126.80 mmol, 3.5 eq) in THF (20 mL) was added 4-(3-fluorophenyl)-4-methylpyrrolidin-2-one (7 g, 36.23 mmol, 1 eq) at 0° C. under N₂. The reaction mixture was stirred at 0° C. for 0.5 h under N₂. TLC (PE:EtOAc=0:1, Rf=0.5) indicated was consumed and many new spots formed. After the reaction mixture was cooled to 0° C., the reaction mixture was quenched by addition of (5 mL) of H₂O, followed by 5 mL of 15% aqueous NaOH and 10 mL of H₂O. After being stirred at room temperature for 1 hour, the solid was removed by filtration. The filtrate was concentrated to dryness to give crude product. The residue was purified by flash silica gel chromatography (ISCO®; 40 g SepaFlash® Silica Flash Column, Eluent of 0~10% MeOH:DCM @100 mL/min) to give compound 3-(3-fluorophenyl)-3-methyl-pyrrolidine (5.7 g, 88% yield) as a yellow oil.

¹H NMR (400 MHz, CHLOROFORM-d) δ=7.28 (dt, J=6.4, 8.0 Hz, 1H), 7.08 (d, J=8.0 Hz, 1H), 7.00 (td, J=2.0, 10.8 Hz, 1H), 6.90 (dt, J=2.0, 8.4 Hz, 1H), 3.23-3.06 (m, 3H), 3.06-3.01 (m, 1H), 2.57 (s, 1H), 2.13 (td, J=8.0, 12.8 Hz, 1H), 1.93 (ddd, J=4.8, 7.6, 12.8 Hz, 1H), 1.36 (s, 3H).

### Step 5: methyl 6-[3-(3-fluorophenyl)-3-methyl-pyrrolidin-1-yl]-8-methyl triazolo[1,5-a]pyridine-2-carboxylate

A mixture of 3-(3-fluorophenyl)-3-methyl-pyrrolidine (1.46 g, 8.15 mmol, 1.1 eq), methyl 6-bromo-8-methyl-[1,2,4]triazolo[1,5-a]pyridine-2-carboxylate (2 g, 7.41 mmol, 1 eq), Cs₂CO₃ (7.24 g, 22.22 mmol, 3 eq), 1,3-bis[2,6-bis(1-propylbutyl)phenyl]-4,5-dichloro-2H-imidazol-1-ium-2-ide;3-chloropyridine;dichloropalladium (144.07 mg, 148.10 μmol, 0.02 eq) and 4A MS (300 mg) in dioxane (20 mL) was degassed and purged with N₂ for 3 times, and then the mixture was stirred at 100° C. for 2 h under N₂ atmosphere. LCMS showed starting material was consumed and the desired mass was detected. The reaction solution was filtered and the filtrate was concentrated under the reduced pressure to give the residue. The residue was purified by flash silica gel chromatography (ISCO®; 40 g SepaFlash® Silica Flash Column, Eluent of 0~50% EtOAc:PE @80 mL/min) to give methyl 6-[3-(3-fluorophenyl)-3-methyl-pyrrolidin-1-yl]-8-methyl-[1,2,4]triazolo[1,5-a]pyridine-2-carboxylate (1.2 g, 42% yield) as a yellow gum.

LC-MS [ESI, M+1]: 369.0.

### Step 6: methyl 6-[(3R)-3-(3-fluorophenyl)-3-methyl-pyrrolidin-1-yl]-8-methyl-[1,2,4]triazolo[1,5-a]pyridine-2-carboxylate

The was methyl 6-[3-(3-fluorophenyl)-3-methyl-pyrrolidin-1-yl]-8-methyl-[1,2,4]triazolo[1,5-a]pyridine-2-carboxylate (1.2 g, 3.26 mmol, 1 eq) purified by SFC by (column: DAICEL CHIRALPAK AD (250 mm*30 mm, 10 um);mobile phase: [C02-MeOH/ACN]; B %: 55%, isocratic elution mode). The reaction solution was concentrated under the reduced pressure to give compound methyl 6-[(3R)-3-(3-fluorophenyl)-3-methyl-pyrrolidin-1-yl]-8-methyl-[1,2,4]triazolo[1,5-a]pyridine-2-carboxylate (550 mg, 46% yield, 100 e.e %)

LC-MS [ESI, M+1]: 369.0.

### Step 7: 6-[(3R)-3-(3-fluorophenyl)-3-methyl-pyrrolidin-1-yl]-8-methyl-[1,2,4]triazolo[1,5-a]pyridine-2-carboxylic acid

To a solution of methyl 6-[(3R)-3-(3-fluorophenyl)-3-methyl-pyrrolidin-1-yl]-8-methyl-[1,2,4]triazolo[1,5-a]pyridine2-carboxylate (550 mg, 1.49 mmol, 1 eq) in THF (5 mL) and H₂O (1 mL) and MeOH (2 mL) was added LiOH·H₂O (187.93 mg, 4.48 mmol, 3 eq). The mixture was stirred at 20° C. for 1 h. LCMS showed starting material was consumed and the desired mass was detected. Acidify the clear solution with diluted hydrochloride acid. The reaction mixture was quenched by water (3 mL) and extracted with EtOAc (4 mL×3), The combined organic layers were washed with brine (12 mL×3), dried over anhydrous Na2SO4, filtered and dried to give compound 6-[(3R)-3-(3-fluorophenyl)-3-methyl-pyrrolidin-1-yl]-8-methyl-[1,2,4]triazolo[1,5-a]pyridine-2-carboxylic acid (500 mg, 95% yield) as a yellow solid.

LC-MS [ESI, M+1]: 335.0.

¹H NMR ¹H NMR (400 MHz, DMSO-d₆) δ=7.92 (d, J=1.6 Hz, 1H), 7.45-7.35 (m, 1H), 7.31 (s, 1H), 7.26-7.17 (m, 2H), 7.11-7.01 (m, 1H), 3.61-3.57 (m, 1H), 3.52-3.46 (m, 2H), 3.37 (br dd, J=3.2, 5.6 Hz, 1H), 2.53 (s, 3H), 2.31-2.19 (m, 2H), 1.35 (s, 3H).

### Step 8: 6-[(3R)-3-(3-fluorophenyl)-3-methyl-pyrrolidin-1-yl]-N, 8-dimethyl-[1,2,4]triazolo[1,5-a]pyridine-2-carboxamide

To a solution of 6-[(3R)-3-(3-fluorophenyl)-3-methyl-pyrrolidin-1-yl]-8-methyl-[1,2,4]triazolo[1,5-a]pyridine-2-carboxylic acid (20 mg, 56.44 μmol, 1 eq) and methanamine;hydrochloride (7.62 mg, 112.87 μmol, 2 eq) in DMF (0.5 mL) was added HATU (42.92 mg, 112.87 μmol, 2 eq) at 0° C. After addition, the mixture was stirred at this temperature for 0.5 h, and DIEA (36.47 mg, 282.19 μmol, 49.15 μL, 5 eq) was added dropwise at 0° C. The resulting mixture was stirred at 20° C. for 1.5 h. LCMS showed starting material was consumed and the desired mass was detected. The reaction was concentrated under reduced pressure to give a residue. The residue was purified by prep-HPLC (column: Phenomenex luna C18 150*25 mm*10 um; mobile phase: [water (FA)—ACN]; gradient: 38%-68% B over 10 min.) and lyophilized to give compound 6-[(3R)-3-(3-fluorophenyl)-3-methyl-pyrrolidin-1-yl]-N, 8-dimethyl-[1,2,4]triazolo[1,5-a]pyridine-2-carboxamide (17.05 mg, 81% yield) as a yellow solid.

LC-MS [ESI, M+1]: 368.0

¹H NMR (400 MHz, CHLOROFORM-d) δ=7.92 (s, 1H), 7.69-7.54 (m, 2H), 7.36 (d, J=7.6 Hz, 1H), 7.32-7.26 (m, 2H), 7.22 (dt, J=2.0, 8.4 Hz, 1H), 3.84 (d, J=8.8 Hz, 1H), 3.82-3.75 (m, 1H), 3.73 (d, J=8.8 Hz, 1H), 3.68 (dt, J=3.6, 8.8 Hz, 1H), 3.33 (d, J=5.2 Hz, 3H), 2.88 (s, 3H), 2.62 (td, J=8.0, 12.0 Hz, 1H), 2.55-2.47 (m, 1H), 1.73 (s, 3H)

| Cmpd. No. | Synthesis | LC/MS | ¹H NMR |
|---|---|---|---|
| 381 | Similar to | m/z 391.1 | ¹H NMR (400 MHz, CHLOROFORM-d) δ = 7.63 (s, |
| | Example 32 | [M + H]⁺ | 1H), 7.40-7.33 (m, 2H), 7.33-7.29 (m, 2H), 7.26-7.23 |
| | | | (m, 1H), 6.94 (s, 1H), 3.86 (s, 2H), 3.58 (br d, J = 8.8 |
| | | | Hz, 1H), 3.53-3.43 (m, 2H), 3.38 (dt, J = 3.7, 8.5 Hz, |
| | | | 1H), 2.89 (br s, 1H), 2.63 (s, 3H), 2.48-2.39 (m, 4H), |
| | | | 2.38-2.31 (m, 1H), 2.28-2.21 (m, 1H), 1.46 (s, 3H) |
| 382 | Similar to | m/z 425.9 | ¹H NMR (400 MHz, CHLOROFORM-d) δ = 7.76 (br s, 1H), |
| | Example 32 | [M + H]⁺ | 7.66 (s, 1H), 7.33 (dt, J = 6.4, 8.0 Hz, 1H), 7.09 |
| | | | (d, J = 8.0 Hz, 1H), 7.05-6.99 (m, 2H), 6.96 (dt, J = |
| | | | 2.4, 8.4 Hz, 1H), 3.59 (d, J = 8.8 Hz, 1H), 3.57-3.49 |
| | | | (m, 3H), 3.47 (d, J = 8.8 Hz, 1H), 3.42 (dt, J = 3.6, 8.8 |
| | | | Hz, 1H), 2.65 (s, 3H), 2.37 (td, J = 8.4, 12.4 Hz, 1H), |
| | | | 2.28-2.22 (m, 1H), 1.47 (s, 3H), 1.33 (s, 6H) |
| 383 | Similar to | m/z 423.9 | ¹H NMR (400 MHz, CHLOROFORM-d) δ = 7.66 (s, |
| | Example 32 | [M + H]⁺ | 1H), 7.53 (br d, J = 8.0 Hz, 1H), 7.36-7.29 (m, 1H), |
| | | | 7.09 (br d, J = 7.6 Hz, 1H), 7.04-6.92 (m, 3H), 4.81 (br |
| | | | d, J = 4.4 Hz, 1H), 4.04 (q, J = 7.6 Hz, 1H), 3.95 (dd, |
| | | | J = 5.4, 9.3 Hz, 1H), 3.90-3.81 (m, 2H), 3.57 (d, J = 8.8 |
| | | | Hz, 1H), 3.55-3.49 (m, 1H), 3.46 (br d, J = 8.8 Hz, 1H), |
| | | | 3.41 (dt, J = 3.6, 8.8 Hz, 1H), 2.62 (s, 3H), 2.43-2.32 |
| | | | (m, 2H), 2.28-2.21 (m, 1H), 2.01 (br dd, J = 4.4, 8.4 |
| | | | Hz, 1H), 1.46 (s, 3H) |
| 384 | Similar to | m/z 573.1 | ¹H NMR (400 MHz, DMSO-d₆) δ = 8.64 (br d, J = 8.0 |
| | Example 32 | [M + H]⁺ | Hz, 1H), 7.87 (s, 1H), 7.40 (q, J = 7.2 Hz, 1H), 7.31 (s, |
| | | | 1H), 7.23 (br d, J = 8.8 Hz, 2H), 7.07 (br t, J = 8.4 Hz, |
| | | | 1H), 4.33 (sxt, J = 8.4 Hz, 1H), 3.63-3.56 (m, 1H), 3.55- |
| | | | 3.43 (m, 3H), 3.38 (br s, 5H), 2.55 (s, 3H), 2.37-2.31 |
| | | | (m, 1H), 2.29-2.17 (m, 6H), 2.14-1.88 (m, 5H), 1.78 |
| | | | (br s, 1H), 1.35 (s, 3H) |
| 385 | Similar to | m/z 438.0 | ¹H NMR (400 MHz, CHLOROFORM-d) δ = 7.65 (s, |
| | Example 32 | [M + H]⁺ | 1H), 7.33 (q, J = 7.5 Hz, 1H), 7.09 (br d, J = 7.6 Hz, 1H), |
| | | | 7.05-6.89 (m, 3H), 4.27-4.06 (m, 2H), 3.96-3.81 (m, |
| | | | 2H), 3.65-3.55 (m, 2H), 3.55-3.49 (m, 1H), 3.49-3.37 |
| | | | (m, 2H), 2.63 (s, 3H), 2.40-2.30 (m, 1H), 2.29-2.20 |
| | | | (m, 1H), 2.11-1.97 (m, 2H), 1.49 (br d, J = 12.8 Hz, |
| | | | 3H), 1.46 (s, 3H) |
| 386 | Similar to | m/z 580.3 | ¹H NMR (400 MHz, DMSO-d₆) δ = 9.26-9.14 (m, |
| | Example 32 | [M + H]⁺ | 1H), 8.03 (br d, J = 8.4 Hz, 1H), 7.68-7.56 (m, 1H), |
| | | | 7.49-7.40 (m, 2H), 7.40-7.31 (m, 2H), 7.30-7.17 (m, |
| | | | 2H), 6.72-6.60 (m, 1H), 3.95-3.95 (m, 1H), 4.00-3.89 |
| | | | (m, 1H), 3.83-3.74 (m, 2H), 3.70 (br t, J = 6.8 Hz, 1H), |
| | | | 3.64-3.44 (m, 5H), 2.62-2.52 (m, 4H), 2.38-2.25 (m, |
| | | | 1H), 2.12-1.92 (m, 4H), 1.55 (d, J = 7.2 Hz, 3H) |
| 387 | Similar to | m/z 580.0 | ¹H NMR (400 MHz, CHLOROFORM-d) δ = 8.99 (s, |
| | Example 32 | [M + H]⁺ | 1H), 7.81-7.69 (m, 1H), 7.69-7.62 (m, 1H), 7.52-7.46 |
| | | | (m, 1H), 7.45-7.37 (m, 2H), 7.35-7.28 (m, 3H), 6.63 |
| | | | (t, J = 8.4 Hz, 1H), 4.27-4.10 (m, 2H), 4.03-3.90 (m, |
| | | | 3H), 3.88-3.73 (m, 1H), 3.71-3.45 (m, 4H), 3.12 (dd, |
| | | | J = 7.2, 16.6 Hz, 1H), 2.81 (dd, J = 7.2, 16.6 Hz, 1H), |
| | | | 2.70 (d, J = 10.0 Hz, 3H), 2.25-2.14 (m, 1H), 2.13- |
| | | | 2.01 (m, 3H), 1.60 (d, J = 7.6 Hz, 3H) |
| 388 | Similar to | m/z 588.1 | ¹H NMR (400 MHz, CHLOROFORM-d) δ = 7.71-7.59 |
| | Example 32 | [M + H]⁺ | (m, 2H), 7.50 (d, J = 7.2 Hz, 1H), 7.24-7.16 (m, 1H), |
| | | | 7.00 (s, 1H), 6.91-6.81 (m, 2H), 6.63 (d, J = 8.4 Hz, |
| | | | 1H), 4.39 (br dd, J = 7.2, 12.0 Hz, 1H), 4.16-3.97 (m, |
| | | | 2H), 3.91-3.74 (m, 3H), 3.64 (dd, J = 3.6, 9.2 Hz, 1H), |
| | | | 3.60-3.46 (m, 4H), 3.41 (dt, J = 2.4, 8.8 Hz, 1H), |
| | | | 3.26-3.05 (m, 2H), 2.64 (s, 3H), 2.48-2.38 (m, 1H), |
| | | | 2.37-2.30 (m, 1H), 1.43 (s, 3H) |
| 389 | Similar to | m/z 588.1 | ¹H NMR (400 MHz, CHLOROFORM-d) δ = 7.74-7.60 |
| | Example 32 | [M + H]⁺ | (m, 2H), 7.50 (d, J = 7.2 Hz, 1H), 7.25-7.16 (m, 1H), |
| | | | 7.03 (s, 1H), 6.91 (br t, J = 9.0 Hz, 2H), 6.64 (d, J = 8.4 |
| | | | Hz, 1H), 4.39 (br dd, J = 7.2, 12.0 Hz, 1H), 4.15-3.94 |
| | | | (m, 2H), 3.89-3.69 (m, 4H), 3.65-3.45 (m, 4H), 3.38 |
| | | | (br t, J = 9.0 Hz, 1H), 3.18 (br d, J = 4.0 Hz, |
| | | | 2H), 2.65 (s, 4H), 2.53-2.38 (m, 1H), 1.43 (s, 3H) |

### Example 33

### 6-(3-ethyl-3-phenyl-azetidin-1-yl)-N, 8-dimethyl-[1,2,4]triazolo[1,5-a]pyridine-2-carboxamide (Compound 33)

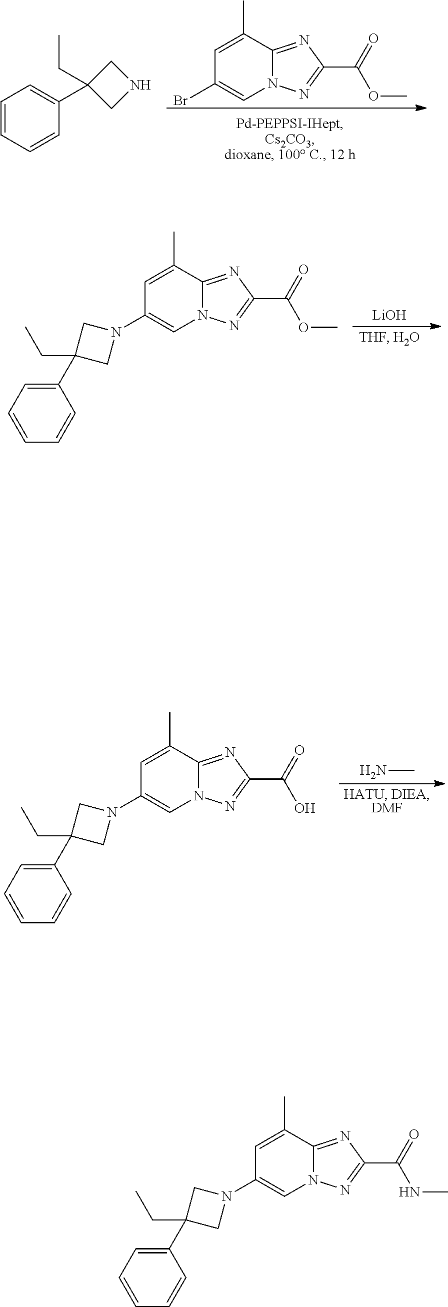

### Step 1: methyl 6-(3-ethyl-3-phenyl-azetidin-1-yl)-8-methyl-[1,2,4]triazolo[1,5-a]pyridine-2-carboxylate

A mixture of 3-ethyl-3-phenyl-azetidine (80 mg, 496.15 μmol, 1 eq), methyl 6-bromo-8-methyl-[1,2,4]triazolo[1,5-a]pyridine-2-carboxylate (134.00 mg, 496.15 μmol, 1 eq), Pd-PEPPSI-IHeptCl (24.13 mg, 24.81 μmol, 0.05 eq), Cs₂CO₃ (484.96 mg, 1.49 mmol, 3 eq) in dioxane (2 mL) was degassed and purged with N₂ for 3 times, and then the mixture was stirred at 100° C. for 2 h under N₂ atmosphere. LCMS showed starting material was consumed completely and the desired compound was detected. The reaction mixture was extracted with EtOAc (10 mL×2).The combined organic phase was washed with brine (10 mL), dried with anhydrous Na2SO4, filtered and concentrated in vacuum. The residue was purified by column chromatography (SiO2, PE:EtOAc=3:1). Compound methyl 6-(3-ethyl-3-phenyl-azetidin-1-yl)-8-methyl-[1,2,4]triazolo[1,5-a]pyridine-2-carboxylate (52 mg, 30% yield) was obtained as a yellow solid.

LC-MS [ESI, M+1]:351.2

### Step 2: 6-(3-ethyl-3-phenyl-azetidin-1-yl)-8-methyl-[1,2,4]triazolo[1,5-a]pyridine-2-carboxylic acid

To a solution of methyl 6-(3-ethyl-3-phenyl-azetidin-1-yl)-8-methyl-[1,2,4]triazolo[1,5-a]pyridine-2-carboxylate (52 mg, 148.40 μmol, 1 eq) in MeOH (0.5 mL) was added LiOH·H₂O (12.45 mg, 296.79 μmol, 2 eq). The mixture was stirred at 25° C. for 2 h. LCMS showed starting material was consumed completely and the desired compound was detected. The reaction was concentrated under reduced pressure to give compound 6-(3-ethyl-3-phenyl-azetidin-1-yl)-8-methyl-[1,2,4]triazolo[1,5-a]pyridine-2-carboxylic acid (30 mg, 60% yield) was obtained as a white solid.

LC-MS [ESI, M+1]:337.1

### Step 3: 6-(3-ethyl-3-phenyl-azetidin-1-yl)-N, 8-dimethyl-[1,2,4]triazolo[1,5-a]pyridine-2-carboxamide

To a solution of 6-(3-ethyl-3-phenyl-azetidin-1-yl)-8-methyl-[1,2,4]triazolo[1,5-a]pyridine-2-carboxylic acid (30 mg, 89.18 μmol, 1 eq) in DMF (1 mL) was added HATU (67.82 mg, 178.37 μmol, 2 eq) and DIEA (23.05 mg, 178.37 μmol, 31.07 μL, 2 eq), methanamine;hydrochloride (9.03 mg, 133.77 μmol, 1.5 eq). The mixture was stirred at 25° C. for 2 h. LCMS showed starting material was consumed completely and the desired compound was detected. The reaction was concentrated under reduced pressure to give a residue. The residue was purified by prep-HPLC (column: Phenomenex luna C18 150*25 mm*10 um; mobile phase: [water(FA)—ACN]; gradient:388%-68% B over 10 min) and lyophilizated to give the product. The mixture was stirred at 25° C. for 2 h. Compound 6-(3-ethyl-3-phenyl-azetidin-1-yl)-N, 8-dimethyl-[1,2,4]triazolo[1,5-a]pyridine-2-carboxamide (8.47 mg, 27% yield) was obtained as a white solid.

¹H NMR (400 MHz, CHLOROFORM-d) δ=7.60 (s, 1H), 7.29 (s, 1H), 7.25 (s, 1H), 7.19-7.13 (m, 1H), 7.05 (d, J=7.6 Hz, 2H), 6.72 (s, 1H), 4.04-3.92 (m, 4H), 2.97 (d, J=5.2 Hz, 3H), 2.49 (s, 3H), 2.01 (q, J=7.6 Hz, 2H), 0.75 (t, J=7.6 Hz, 3H)

LC-MS [ESI, M+1]: 350.1

| Cmpd. No. | Synthesis | LC/MS | ¹H NMR |
|---|---|---|---|
| 390 | Similar to | m/z 540.2 | ¹H NMR (400 MHz, CHLOROFORM-d) δ = 7.60 (t, |
| | Example 33 | [M + H]+ | J = 7.9 Hz, 1H), 7.62-7.50 (m, 2H), 7.42-7.32 (m, |
| | | | 2H), 7.26-7.20 (m, 1H), 7.14 (d, J = 7.6 Hz, 2H), 6.98 |
| | | | (d, J = 8.4 Hz, 1H), 6.81 (s, 1H), 4.45-4.19 (m, 3H), |
| | | | 4.17-3.99 (m, 4H), 3.32-3.02 (m, 2H), 2.58 (s, 3H), |
| | | | 2.22 (d, J = 12.0 Hz, 2H), 2.10 (q, J = 6.8 Hz, 2H), |
| | | | 1.66 (d, J = 10.3 Hz, 2H), 0.84 (br t, J = 7.2 Hz, 3H) |
| 391 | Similar to | m/z 366 | ¹H NMR (400 MHz, CHLOROFORM-d) δ = 7.6 (s, |
| | Example 33 | [M + H]+ | 1H), 7.40-7.32 (m, 2H), 7.26-7.21 (m, 1H), 7.13 (d, |
| | | | J = 7.6 Hz, 2H), 7.07 (s, 1H), 5.02 (s, 2H), 4.15-4.00 |
| | | | (m, 4H), 3.40 (br s, 1H), 3.05 (d, J = 5.0 Hz, 3H), 2.09 |
| | | | (q, J = 7.2 Hz, 2H), 0.83 (t, J = 7.2 Hz, 3H) |
| 392 | Similar to | m/z 556.3 | ¹H NMR (400 MHz, CHLOROFORM-d) δ = 7.6- |
| | Example 33 | [M + H]+ | 7.67 (m, 1H), 7.62 (s, 1H), 7.54 (d, J = 7.2 Hz, 1H), |
| | | | 7.40-7.33 (m, 2H), 7.26-7.21 (m, 1H), 7.19 (d, J = |
| | | | 8.4 Hz, 1H), 7.14 (d, J = 7.6 Hz, 2H), 7.06 (s, 1H), |
| | | | 6.98 (d, J = 8.8 Hz, 1H), 5.01 (s, 2H), 4.45-4.23 (m, |
| | | | 3H), 4.17-4.01 (m, 4H), 3.18 (t, J = 12.4 Hz, 2H), |
| | | | 2.22 (d, J = 11.4 Hz, 2H), 2.10 (q, J = 7.2 Hz, 2H), |
| | | | 1.66 (s, 2H), 0.84 (t, J = 7.2 Hz, 3H) |
| 393 | Similar to | m/z 552.2 | ¹H NMR (400 MHz, CHLOROFORM-d) δ = 7.73- |
| | Example 33 | [M + H]+ | 7.62 (m, 1H), 7.57 (d, J = 1.6 Hz, 1H), 7.49 (d, J = 7.2 |
| | | | Hz, 1H), 7.40-7.31 (m, 2H), 7.26-7.20 (m, 1H), 7.13 |
| | | | (d, J = 7.2 Hz, 2H), 6.80 (s, 1H), 6.63 (d, J = 8.5 Hz, |
| | | | 1H), 4.36 (dd, J = 7.2, 12.2 Hz, 1H), 4.12-3.97 (m, |
| | | | 6H), 3.86-3.74 (m, 3H), 3.58-3.45 (m, 2H), 3.24- |
| | | | 3.12 (m, 2H), 2.60 (s, 3H), 2.10 (q, J = 7.3 Hz, 2H), |
| | | | 0.83 (t, J = 7.2 Hz, 3H) |
| 394 | Similar to | m/z 566.2 | ¹H NMR (400 MHz, CHLOROFORM-d) δ = 7.70- |
| | Example 33 | [M + H]+ | 7.62 (m, 1H), 7.58 (d, J = 13.1 Hz, 1H), 7.49 (t, J = |
| | | | 6.4 Hz, 1H), 7.40-7.31 (m, 2H), 7.26-7.20 (m, 1H), |
| | | | 7.13 (t, J = 7.6 Hz, 2H), 6.81 (d, J = 9.5 Hz, 1H), 6.62 |
| | | | (t, J = 8.9 Hz, 1H), 4.27-4.14 (m, 1H), 4.13-4.02 (m, |
| | | | 4H), 4.00 (s, 1H), 3.91 (t, J = 7.0 Hz, 1H), 3.87-3.73 |
| | | | (m, 1H), 3.71-3.45 (m, 4H), 2.60 (d, J = 11.2 Hz, 3H), |
| | | | 2.19-2.04 (m, 6H), 0.90-0.77 (m, 3H) |
| 395 | Similar to | m/z 540.2 | ¹H NMR (400 MHz, CHLOROFORM-d) δ = 8.4 (s, |
| | Example 33 | [M + H]+ | 1H), 7.67 (d, J = 6.4 Hz, 1H), 7.59 (s, 1H), 7.51 (s, |
| | | | 1H), 7.36-7.29 (m, 2H), 7.25-7.18 (m, 1H), 7.11 (d, |
| | | | J = 7.4 Hz, 2H), 6.88 (s, 1H), 6.74 (s, 1H), 4.34 (s, |
| | | | 1H), 4.22-4.09 (m, 2H), 4.07-3.95 (m, 4H), 3.28 (s, |
| | | | 2H), 2.51 (s, 3H), 2.16 (s, 2H), 2.05 (d, J = 7.1 Hz, |
| | | | 2H), 1.71 (d, J = 8.4 Hz, 2H), 0.88-0.72 (m, 3H) |
| 396 | Similar to | m/z 566.2 | ¹H NMR (400 MHz, CHLOROFORM-d) δ = 7.66 (t, |
| | Example 33 | [M + H]+ | J = 7.2 Hz, 1H), 7.56 (s, 1H), 7.50 (d, J = 7.3 Hz, 1H), |
| | | | 7.17 (d, J = 7.3 Hz, 2H), 7.03 (d, J = 7.4 Hz, 2H), 6.79 |
| | | | (s, 1H), 6.63 (d, J = 8.5 Hz, 1H), 4.42-4.32 (m, 1H), |
| | | | 4.11-3.96 (m, 6H), 3.88-3.71 (m, 3H), 3.60-3.44 |
| | | | (m, 2H), 3.17 (s, 2H), 2.60 (s, 3H), 2.34 (s, 3H), |
| | | | 2.13-2.01 (m, 2H), 0.83 (t, J = 6.9 Hz, 3H) |
| 397 | Similar to | m/z 566.2 | ¹H NMR (400 MHz, CHLOROFORM-d) δ ppm 0.83 |
| | Example 33 | [M + H]⁺ | (t, J = 7.2 Hz, 3 H) 2.09 (q, J = 7.2 Hz, 2 H) 2.36 (s, 3 H) |
| | | | 2.60 (s, 3 H) 3.17 (br d, J = 3.6 Hz, 2 H) 3.45-3.60 (m, |
| | | | 2 H) 3.75-3.88 (m, 3 H) 3.96-4.16 (m, 6 H) 4.37 (br dd, J = |
| | | | 10.4, 6.25 Hz, 1 H) 6.63 (d, J = 8.4 Hz, 1 H) 6.80 |
| | | | (s, 1 H) 6.89-6.97 (m, 2 H) 7.05 (br d, J = 7.2 Hz, 1 H) |
| | | | 7.21-7.26 (m, 1 H) 7.50 (d, J = 7.2 Hz, 1 H) 7.57 (br |
| | | | s, 1 H) 7.66 (t, J = 7.6 Hz, 1 H) |
| 398 | Similar to | m/z 552.3 | ¹H NMR (400 MHz, CD3OD, 296 K) δ (ppm) = 7.89- |
| | Example 33 | [M + H]⁺ | 7.65 (m, 2H), 7.41-7.18 (m, 6H), 7.11-7.03 (m, |
| | | | 1H), 6.83 (m, J = 8.8, 17.7 Hz, 1H), 4.21-3.97 (m, |
| | | | 6H), 3.90-3.82 (m, 1H), 3.79-3.56 (m, 5H), 2.57 (d, |
| | | | J = 14.4 Hz, 3H), 2.27-2.03 (m, 4H), 1.73 (d, J = 8.4 |
| | | | Hz, 3H) |
| 399 | Similar to | m/z 538.3 | ¹H NMR (400 MHz, CD3OD, 299 K) δ (ppm) = 7.81 |
| | Example 33 | [M + H]⁺ | (d, J = 2.0 Hz, 1H), 7.73 (m, J = 7.3, 8.5 Hz, 1H), |
| | | | 7.41-7.17 (m, 6H), 7.07 (s, 1H), 6.83 (d, J = 8.4 Hz, |
| | | | 1H), 4.36-4.25 (m, 1H), 4.13-4.08 (m, 2H), 4.06-3.98 |
| | | | (m, 4H), 3.90-3.79 (m, 2H), 3.72 (m, J = 4.1, 13.0 |
| | | | Hz, 1H), 3.58 (m, J = 4.2, 11.4, 15.7 Hz, 2H), 3.26- |
| | | | 3.18 (m, 2H), 2.57 (s, 3H), 1.74 (s, 3H) |

### Example 34

### 6-[4-[[8-methyl-6-[3-methyl-3-(2-pyridyl)pyrrolidin-1-yl]-[1,2,4]triazolo[1,5-a]pyridine-2-carbonyl]amino]-1-piperidyl]pyridine-2-carboxylic acid (Compound 34)

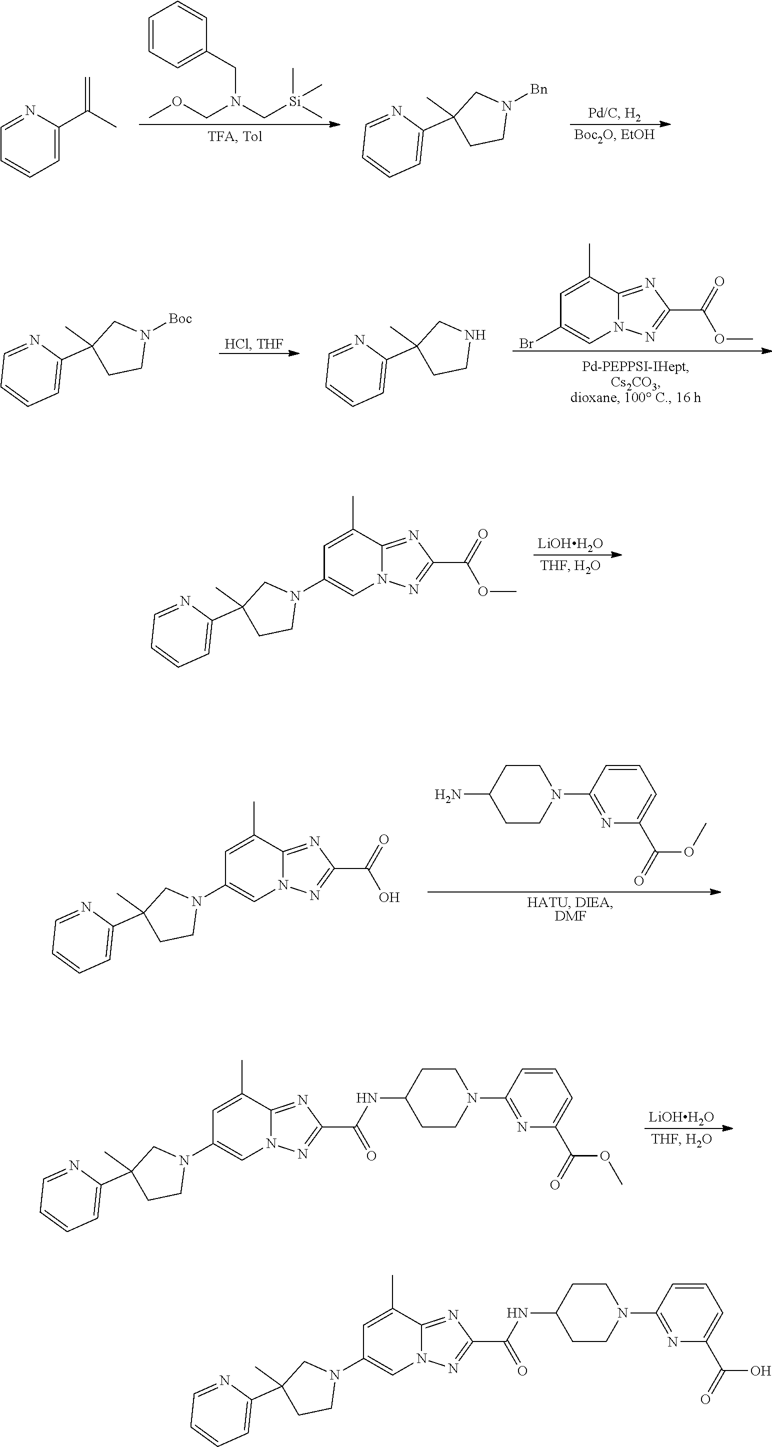

### 2-(1-benzyl-3-methylpyrrolidin-3-yl)pyridine

To a mixture of 2-isopropenylpyridine (5 g, 41.96 mmol, 1 eq) and N-(methoxymethyl)-1-phenyl-N-(trimethylsilylmethyl)methanamine (8.97 g, 37.76 mmol, 0.9 eq) in Tol. (50 mL) was added 2,2,2-trifluoroacetic acid (478.43 mg, 4.20 mmol, 311.68 μL, 0.1 eq), and then the mixture was stirred at 25° C. for 48 h. LCMS and TLC (DCM:MeOH=20:1, Rf=0.23) indicated reactant was consumed and one new spot formed. The reaction mixture was diluted with H₂O 100 mL and extracted with EtOAc 100 mL (100 mL×3). The combined organic layers were dried over Na2SO4, filtered and concentrated under reduced pressure to give a residue. The residue was purified by column chromatography (SiO₂, DCM:MeOH=99:1 to 90:10) to give the Compound 2-(1-benzyl-3-methylpyrrolidin-3-yl) pyridine (3.56 g, 34% yield) was obtained as a light yellow oil,

LC-MS [ESI, M+1]: 253.1.

¹H NMR (400 MHz, CHLOROFORM-d) δ=8.50 (br d, J=4.4 Hz, 1H), 7.61-7.50 (m, 1H), 7.37-7.30 (m, 1H), 7.29 (br s, 1H), 7.30-7.24 (m, 1H), 7.25 (br s, 1H), 7.20 (br d, J=7.2 Hz, 1H), 7.23-7.16 (m, 1H), 7.08-7.00 (m, 1H), 7.08-6.97 (m, 1H), 3.65 (s, 1H), 2.96 (d, J=9.2 Hz, 1H), 2.91-2.77 (m, 1H), 2.71 (d, J=9.2 Hz, 1H), 2.66-2.57 (m, 1H), 2.45-2.35 (m, 1H), 1.95 (br d, J=7.2 Hz, 1H), 1.46 (s, 1H).

### Step 2: tert-butyl 3-methyl-3-(pyridin-2-yl) pyrrolidine-1-carboxylate

To a solution of 2-(1-benzyl-3-methyl-pyrrolidin-3-yl) pyridine (600 mg, 2.38 mmol, 1 eq) and Boc₂O (778.36 mg, 3.57 mmol, 819.33 μL, 1.5 eq) in EtOH (30 mL) was added Pd/C (126.51 mg, 118.88 μmol, 10% purity, 0.05 eq) and Pd(OH)₂ (166.95 mg, 118.88 μmol, 10% purity, 0.05 eq) under N₂ atmosphere. The suspension was degassed and purged with H2 (4.80 mg, 2.38 mmol, 1 eq) for 3 times. The mixture was stirred under H2 (4.80 mg, 2.38 mmol, 1 eq) (15 Psi) at 25° C. for 1 h. The reaction was monitored by LCMS indicated reactant was consumed was consumed and no desired mass was detected. The reaction mixture was filtered. Without purification. Compound tert-butyl 3-methyl-3-(2-pyridyl) pyrrolidine-1-carboxylate (800 mg, crude) was obtained as a yellow oil.

LC-MS [ESI, M+1]: 263.5

### Step 3: 2-(3-methylpyrrolidin-3-yl) pyridine

To a mixture of tert-butyl 3-methyl-3-(2-pyridyl) pyrrolidine-1-carboxylate (280 mg, 1.07 mmol, 1 eq) in THF (5 mL) was added hydrogen chloride (1.43 g, 39.17 mmol, 1.40 mL, 36.70 eq), and then the mixture was stirred at 25° C. for 2 h. LCMS and TLC (DCM:MeOH=20:1, Rf=0.19) indicated reactant was consumed and one main peak with desired mass was detected. The reaction mixture was added to the reaction solution with an appropriate amount of sodium hydroxide solid. After the reaction temperature dropped to room temperature, the filtrate was filtered, and methyl tert-butylamine was added and dried. The residue was purified by prep-TLC (SiO₂, DCM:MeOH=20:1, Rf=0.19).to give Compound 2-(3-methylpyrrolidin-3-yl)pyridine (109 mg, 38% yield) was obtained as a yellow oil.

LC-MS [ESI, M+1]: 163.2.

¹H NMR (400 MHz, CHLOROFORM-d) δ=8.61-8.47 (m, 1H), 7.68-7.54 (m, 1H), 7.34-7.23 (m, 3H), 7.14-7.06 (m, 1H), 3.41-3.31 (m, 1H), 3.17-3.06 (m, 2H), 2.96-2.87 (m, 1H), 2.38-2.27 (m, 1H), 1.95-1.83 (m, 1H), 1.50-1.33 (m, 4H).

### Step 4: methyl 8-methyl-6-(3-methyl-3-(pyridin-2-yl)pyrrolidin-1-yl)-[1,2,4]triazolo[1,5-a]pyridine-2-carboxylate

To a mixture of methyl 6-bromo-8-methyl-[1,2,4]triazolo[1,5-a]pyridine-2-carboxylate (181.46 mg, 671.88 μmol, 1 eq) and 2-(3-methylpyrrolidin-3-yl)pyridine (109 mg, 671.88 μmol, 1 eq) in dioxane (1 mL) was added Cs₂CO₃ (656.74 mg, 2.02 mmol, 3 eq) and Pd-PEPPSI-IHeptCl (65.36 mg, 67.19 μmol, 0.1 eq), and then the mixture was stirred at 100° C. for 16 h. LCMS and TLC (DCM:MeOH=20:1, Rf=0.34)showed starting material was consumed completely and one main peak with desired m/z was detected. The reaction mixture was diluted with H₂O 10 mL and extracted with EtOAc 10 mL (10 mL×3). The combined organic layers were dried over Na2SO4, filtered and concentrated under reduced pressure to give a residue. The residue was purified by prep-TLC (SiO₂, DCM:MeOH=20:1, Rf=0.34) and eluted with EtOAc (10 mL) to give the Compound methyl 8-methyl-6-[3-methyl-3-(2-pyridyl)pyrrolidin-1-yl]-[1,2,4]triazolo[1,5-a]pyridine-2-carboxylate (100 mg, 284.57 μmol, 43% yield) was obtained as a light yellow oil.

LC-MS [ESI, M+1]: 352.1.

### Step 5: 8-methyl-6-(3-methyl-3-(pyridin-2-yl)pyrrolidin-1-yl)-[1,2,4]triazolo[1,5-a]pyridine-2-carboxylic acid

To a mixture of methyl 8-methyl-6-[3-methyl-3-(2-pyridyl)pyrrolidin-1-yl]-[1,2,4]triazolo[1,5-a]pyridine-2-carboxylate (100 mg, 284.57 μmol, 1 eq) in THF (2 mL) was added LiOH·H₂O (11.94 mg, 284.57 μmol, 1 eq), and then add H₂O (1 mL) the mixture was stirred at 25° C. for 0.15 h. LC-MS and TLC (DCM:MeOH showed=10:1, Rf=0.12) indicated starting material was consumed and one main peak with desired mass was detected. The reaction mixture was diluted with saturated sodium carbonate 10 mL and extracted with EtOAc 10 mL (10 mL×3). The combined organic layers was washed with saturated brine. It was dried over Na2SO4, filtered and concentrated under reduced pressure to give a residue. The residue was purified by prep-TLC (SiO₂, DCM:MeOH showed=10:1, Rf=0.12) and eluted with EtOAc (20 mL) to give the product. Compound 8-methyl-6-[3-methyl-3-(2-pyridyl)pyrrolidin-1-yl]-[1,2,4]triazolo[1,5-a]pyridine-2-carboxylic acid (70 mg, 73% yield) was yellow liquid,

LC-MS [ESI, M+1]: 381.1.

### Step 6: methyl 6-(4-(8-methyl-6-(3-methyl-3-(pyridin-2-yl)pyrrolidin-1-yl)-[1,2,4]triazolo[1,5-a]pyridine-2-carboxamido)piperidin-1-yl)picolinate

To a mixture of methyl 6-(4-amino-1-piperidyl)pyridine-2-carboxylate (67.09 mg, 285.14 μmol, 1.3 eq) in DMF (1 mL) was added HATU (250.20 mg, 658.02 μmol, 3 eq) and DIEA (141.74 mg, 1.10 mmol, 191.03 μL, 5 eq) and then add 8-methyl-6-[3-methyl-3-(2-pyridyl)pyrrolidin-1-yl]-[1,2,4]triazolo[1,5-a]pyridine-2-carboxylic acid (74 mg, 219.34 μmol, 1 eq) the mixture was stirred at 25° C. for 12 h. LCMS and TLC (DCM:MeOH showed=20:1, Rf=0.23) indicated starting material was consumed and one main peak with desired mass was detected. The reaction mixture was diluted with saturated sodium carbonate 10 mL and extracted with EtOAc 10 mL (10 mL×3). The combined organic layers was washed with saturated brine. It was dried over Na2SO4, filtered and concentrated under reduced pressure to give a residue. The residue was purified by prep-TLC (SiO₂, DCM:MeOH=20:1, Rf=0.23) and eluted with EtOAc (20 mL) to give the Compound methyl 6-[4-[[8-methyl-6-[3-methyl-3-(2-pyridyl)pyrrolidin-1-yl]-[1,2,4]triazolo[1,5-a]pyridine-2-carbonyl]amino]-1-piperidyl]pyridine-2-carboxylate (54 mg, 45% yield) was obtained as a light yellow Liquid.

LC-MS [ESI, M+1]: 555.3

### Step 7: 6-(4-(8-methyl-6-(3-methyl-3-(pyridin-2-yl)pyrrolidin-1-yl)-[1,2,4]triazolo[1,5-a]pyridine-2-carboxamido)piperidin-1-yl)picolinic acid

To a mixture of methyl 6-[4-[[8-methyl-6-[3-methyl-3-(2-pyridyl)pyrrolidin-1-yl]-[1,2,4]triazolo[1,5-a]pyridine-2-carbonyl]amino]-1-piperidyl]pyridine-2-carboxylate (10 mg, 18.03 μmol, 1 eq) and LiOH·H₂O (3.03 mg, 72.12 μmol, 4 eq) in THF (1 mL) was added H₂O (0.5 mL), and then the mixture was stirred at 25° C. for 0.15 h. LCMS indicated starting material was consumed and one main peak with desired mass was detected. The reaction mixture was diluted with H₂O 10 mL and extracted with EtoAc 10 mL (10 mL×3). The combined organic layers were dried over Na2SO4, filtered and concentrated under reduced pressure to give a residue. The residue was purified by prep-HPLC TFA condition; column: Phenomenex luna C18 150×25 mm×10 um; mobile phase: [water (FA)—ACN]; gradient:5%-35% B over 10 min) to give desired compound 6-[4-[[8-methyl-6-[3-methyl-3-(2-pyridyl)pyrrolidin-1-yl]-[1,2,4]triazolo[1,5-a]pyridine-2-carbonyl]amino]-1-piperidyl]pyridine-2-carboxylic acid (0.8 mg, 8% yield) as a white solid.

LC-MS [ESI, M+1]: 541.3.

¹H NMR (400 MHz, CHLOROFORM-d) δ=8.59 (br dd, J=0.8, 3.2 Hz, 1H), 8.59 (br s, 1H), 8.61-8.57 (m, 1H), 8.64-8.55 (m, 1H), 8.69-8.52 (m, 1H), 7.73-7.61 (m, 1H), 7.75-7.60 (m, 1H), 7.74-7.58 (m, 1H), 7.58-7.48 (m, 1H), 7.53 (br d, J=4.0 Hz, 1H), 7.36-7.29 (m, 1H), 7.32 (br d, J=7.6 Hz, 1H), 7.23-7.14 (m, 1H), 7.21-7.11 (m, 1H), 7.09-7.00 (m, 1H), 7.02 (br s, 1H), 6.99-6.91 (m, 1H), 7.00-6.91 (m, 1H), 4.44-4.34 (m, 1H), 4.34-4.23 (m, 1H), 4.53-4.07 (m, 1H), 3.90-3.83 (m, 1H), 3.87 (br d, J=8.8 Hz, 1H), 3.57-3.45 (m, 1H), 3.45-3.37 (m, 1H), 3.64-3.34 (m, 2H), 3.24-3.11 (m, 1H), 3.26-3.01 (m, 1H), 2.60 (br s, 3H), 2.66-2.53 (m, 1H), 2.56 (br s, 1H), 2.28-2.18 (m, 1H), 2.35-2.12 (m, 4H), 1.52 (br s, 3H), 1.64-1.27 (m, 1H).

| | | | |
|---|---|---|---|
| Cmpd. No. | Synthesis | LC/MS | ¹H NMR |

| | | |
|---|---|---|
| 400 | Similar to | m/z 568.2 |
| | Example 34 | [M + H]⁺ |

### Example 35

### 6-[5-[8-methyl-6-[3-methyl-3-(2-methyltetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (Compound 35)

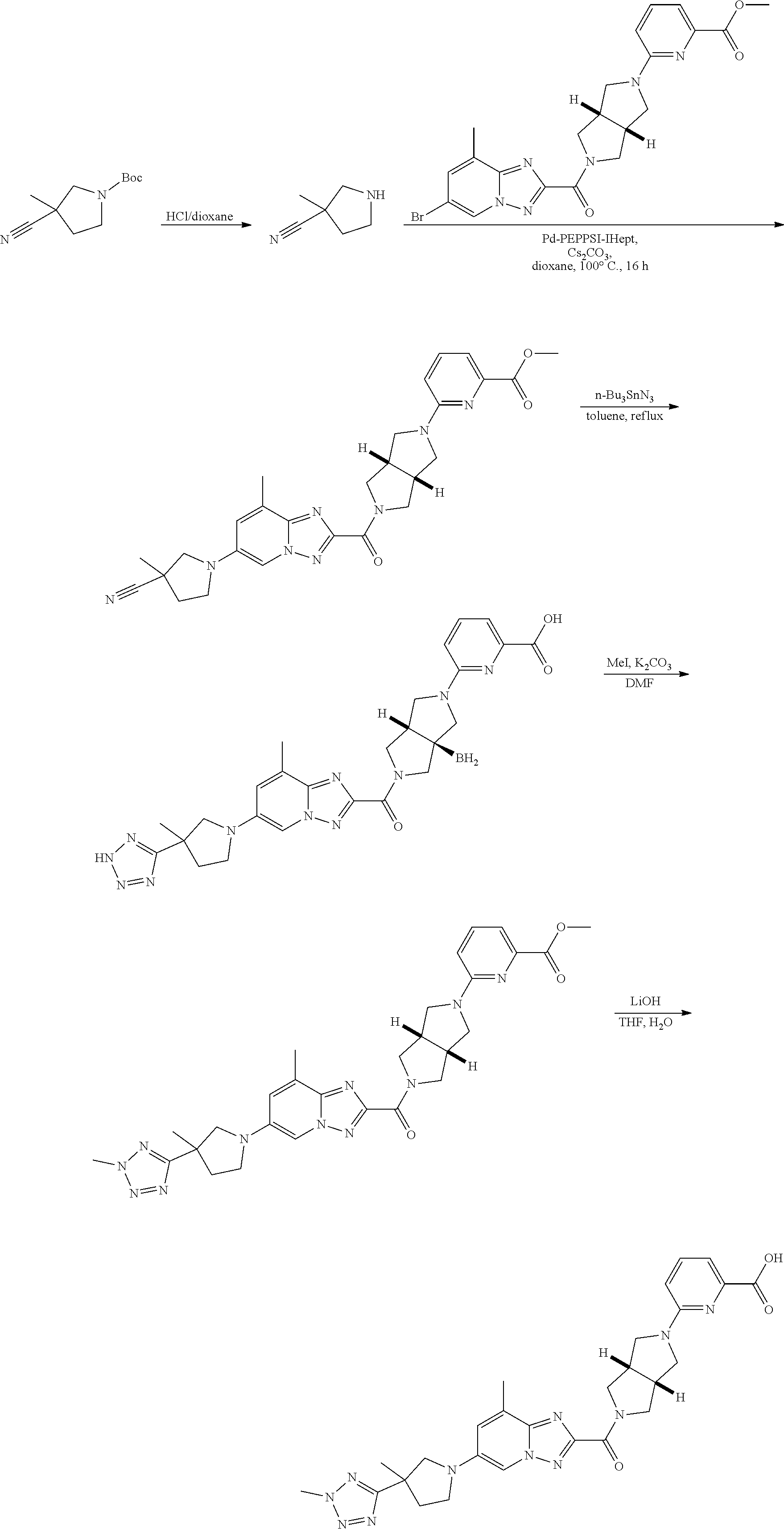

### Step 1: 3-methylpyrrolidine-3-carbonitrile

To a solution of tert-butyl 3-cyano-3-methyl-pyrrolidine-1-carboxylate (1 g, 4.76 mmol, 1 eq) in DCM (15 mL) was added HCl/dioxane (2 M, 2.38 mL, 1 eq).The mixture was stirred at 25° C. for 15 min. LCMS showed starting material was consumed and one main peak with desired mass was detected. The mixture was concentrated in vacuo. The reaction mixture was concentrated under the reduced pressure to give the product to a solution of Cs₂CO₃ (2 eq, 3 g) in THF (10 mL), the mixture was stirred at 40° C. for 2 h to give compound 3-methylpyrrolidine-3-carbonitrile (350 mg, crude) was obtained as a yellow oil.

LC-MS [ESI, M+1]: 111.2.

¹H NMR (DMSO-d₆, 400 MHz): δ=3.10 (d, J=11.2 Hz, 1.0H), 2.91 (t, J=7.2 Hz, 2.0H), 2.75 (d, J=11.2 Hz, 1.0H), 2.04-2.19 (m, 1.0H), 1.73 (dt, J=12.8, 7.2 Hz, 1.0H), 1.35 ppm (s, 3.1H).

### Step 2: methyl 6-[5-[6-(3-cyano-3-methyl-pyrrolidin-1-yl)-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate

To a solution of 3-methylpyrrolidine-3-carbonitrile (203.92 mg, 1.85 mmol, 2.00 eq) and methyl 6-[5-(6-bromo-8-methyl-[1,2,4]triazolo[1,5-a]pyridine-2-carbonyl)-1,3,3a, 4,6,6a-hexahydropyrrolo[3,4-c]pyrrol-2-yl]pyridine-2-carboxylate (450 mg, 927.20 μmol, 1 eq) in dioxane (10 mL) was added Cs₂CO₃ (906.30 mg, 2.78 mmol, 3 eq) and 1,3-bis[2,6-bis(1-propylbutyl)phenyl]-4,5-dichloro-2H-imidazol-1-ium-2-ide;3-chloropyridine;dichloropalladium (90.20 mg, 92.72 μmol, 0.1 eq). The mixture was stirred at 100° C. for 1 h. LCMS showed starting material was consumed and one main peak with desired mass was detected. The mixture was concentrated in vacuo. The residue was purified by prep-TLC (DCM:MeOH=20:1, Rf=0.35) and eluted with 60 mL (10% methanol in DCM) to give the product compound methyl 6-[5-[6-(3-cyano-3-methyl-pyrrolidin-1-yl)-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate (300 mg, 63% yield) was obtained as a white solid.

LC-MS [ESI, M+1]: 515.2.

¹H NMR (DMSO-d₆, 400 MHz): δ=7.03 (d, J=8.0 Hz, 1.0H), 6.01 ppm (dd, J=8.0, 2.0 Hz, 2.0H).

### Step 3: 6-[5-[8-methyl-6-[3-methyl-3-(2H-tetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (Compound 401)

To a solution of methyl 6-[5-[6-(3-cyano-3-methyl-pyrrolidin-1-yl)-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate (130 mg, 252.63 μmol, 1 eq) in toluene (1.5 mL) was added azido (tributyl) stannane (587.25 mg, 1.77 mmol, 7 eq). The mixture was stirred at 120° C. for 1 h. LCMS showed starting material was consumed and one main peak with desired mass was detected. The mixture was concentrated in vacuo. The reaction mixture was concentrated under the reduced pressure to give compound 6-[5-[8-methyl-6-[3-methyl-3-(2H-tetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (130 mg, crude) was obtained as a white gum.

LC-MS [ESI, M+1]: 544.3

### Step 4: 6-[5-[8-methyl-6-[3-methyl-3-(2H-tetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid

To a solution of 6-[5-[8-methyl-6-[3-methyl-3-(2H-tetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (100 mg, 183.97 μmol, 1 eq) in DMF (2 mL) was added MeI (52.22 mg, 367.93 μmol, 22.91 μL, 2 eq) and K₂CO₃ (50.85 mg, 367.93 μmol, 2 eq).The mixture was stirred at 25° C. for 1 h.LCMS showed starting material was consumed and the desired mass was detected. The reaction was quenched in H₂O (10 mL). The residue was purified by prep-TLC (SiO₂, DCM:MeOH (NH3·MeOH 1%)=20:1, Rf=0.2) and eluted with 60 mL (10% methanol in DCM) compound methyl 6-[5-[8-methyl-6-[3-methyl-3-(2-methyltetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylate (100 mg, 95% yield) was obtained as a white solid.

LC-MS [ESI, M+1]: 572.2

### Step 5: 6-[5-[8-methyl-6-[3-methyl-3-(2-methyltetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid

To a solution of methyl 6-[5-[8-methyl-6-[3-methyl-3-(2-methyltetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylate (100 mg, 174.94 μmol, 1 eq) in THF (2 mL) and H₂O (0.3 mL) was added LiOH·H₂O (29.36 mg, 699.75 μmol, 4 eq) The mixture was stirred at 25° C. for 10 min. LCMS showed starting material was consumed and one main peak with desired mass was detected. The reaction mixture was concentrated under the reduced pressure to give the product. The residue was purified by prep-HPLC (column: CD04-Welch Ultimate C18 150*25*7 um; mobile phase: [water(FA)—ACN]; gradient:12%-42% B over 8 min) and lyophilized to give compound 6-[5-[8-methyl-6-[3-methyl-3-(2-methyltetrazol-5-yl)pyrrolidin-1-yl]-[1,2,4]triazolo[1,5-a]pyridine-2-carbonyl]-1,3,3a, 4,6,6a-hexahydropyrrolo[3,4-c]pyrrol-2-yl]pyridine-2-carboxylic acid (20.15 mg, 99% purity) was obtained as a white solid.

LC-MS [ESI, M+1]: 558.7.

¹H NMR (CHLOROFORM-d, 400 MHz): δ=7.62-7.72 (m, 1.0H), 7.60 (s, 1.0H), 7.49 (d, J=7.2 Hz, 1.0H), 6.96 (s, 1.0H), 6.63 (d, J=8.4 Hz, 1.0H), 4.37 (br dd, J=12.0, 7.2 Hz, 1.0H), 4.32 (s, 3.0H), 3.99-4.12 (m, 2.1H), 3.93 (d, J=9.2 Hz, 1.1H), 3.75-3.86 (m, 3.2H), 3.48 (br d, J=2.4 Hz, 3.0H), 3.43 (br t, J=8.8 Hz, 2.3H), 3.11-3.23 (m, 2.2H), 2.70-2.79 (m, 1.1H), 2.61 (s, 3.2H), 2.24 (dt, J=12.4, 7.2 Hz, 1.2H), 1.63 ppm (s, 3.2H).

### Example 36

### (R)-4-(7-(8-methyl-6-(3-methyl-3-phenylpyrrolidin-1-yl)-[1,2,4]triazolo[1,5-a]pyridine-2-carbonyl)-5,6,7,8-tetrahydro-[1,2,4]triazolo[4,3-a]pyrazin-3-yl)picolinic acid (Compound 36)

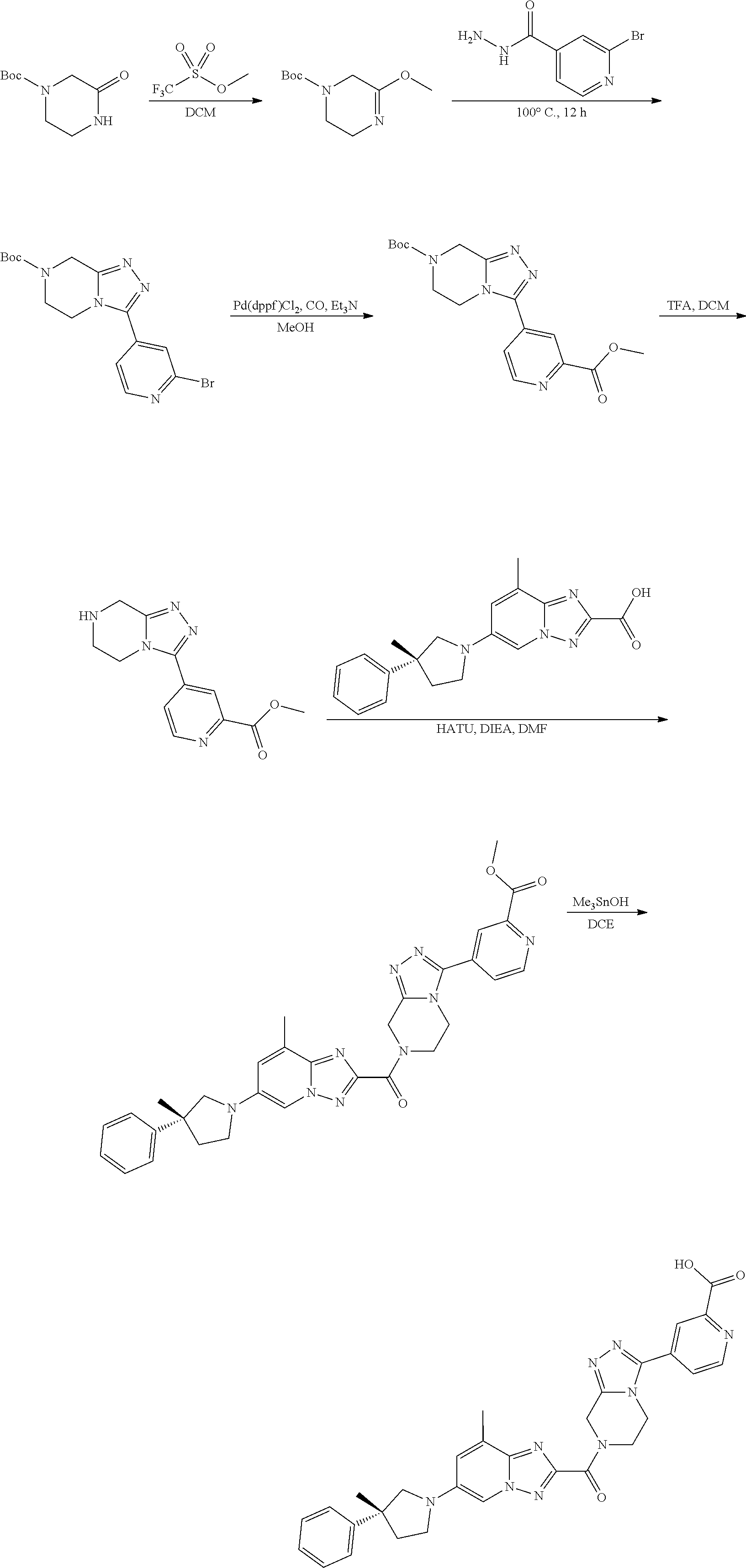

### Step 1: tert-butyl 5-methoxy-3,6-dihydropyrazine-1(2H)-carboxylate

To a solution of tert-butyl 3-oxopiperazine-1-carboxylate (2 g, 9.99 mmol, 1 eq) in DCM (20 mL) was added methyl trifluoromethanesulfonate (3.28 g, 19.98 mmol, 2.19 mL, 2 eq).The mixture was stirred at 40° C. for 2 h. LCMS showed starting material was consumed and one main peak with desired mass was detected. The pH was adjusted to around 7 by progressively aq. NaHCO₃, and then extracted with EtOAc (15 mL×2).The combined organic layers were washed with brine (30 mL), dried over Na2SO4, filtered and concentrated under reduced pressure to give compound tert-butyl 6-methoxy-3,5-dihydro-2H-pyrazine-4-carboxylate (2.3 g, crude) as a yellow oil.

LC-MS [ESI, M-56]: 159.1.

¹H NMR (400 MHz, CHLOROFORM-d) δ=4.14 (s, 3H), 3.90 (br t, J=5.2 Hz, 1H), 3.79 (s, 1H), 3.74 (br d, J=9.2 Hz, 2H), 3.69-3.65 (m, 2H), 1.48 (s, 9H).

### Step 2: tert-butyl 3-(2-bromopyridin-4-yl)-5,6-dihydro-[1,2,4]triazolo[4,3-a]pyrazine-7(8H)-carboxylate

To a solution of tert-butyl 6-methoxy-3,5-dihydro-2H-pyrazine-4-carboxylate (99.18 mg, 462.89 μmol, 2 eq) in EtOH (0.5 mL) was added 2-bromopyridine-4-carbohydrazide (50 mg, 231.44 μmol, 1 eq). The mixture was stirred at 100° C. for 1 h. LCMS showed starting material was consumed and one main peak with desired mass was detected. The reaction was added water (20 mL) and then extracted with DCM (15 mL×3).The combined organic phase was washed with brine The residue was purified by prep-TLC (SiO₂, DCM:MeOH=15:1, Rf=0.60) to give compound tert-butyl 3-(2-bromo-4-pyridyl)-6,8-dihydro-5H-[1,2,4]triazolo[4,3-a]pyrazine-7-carboxylate (400 mg, 18% yield) as a yellow solid.

LC-MS [ESI, M+1]: 380.0.

¹H NMR (400 MHz, CHLOROFORM-d) δ=8.54 (d, J=5.2 Hz, 1H), 7.86 (s, 1H), 7.65 (br d, J=4.8 Hz, 1H), 4.95 (s, 2H), 4.20 (br t, J=5.2 Hz, 2H), 3.91 (br t, J=4.8 Hz, 2H), 1.52 (s, 9H).

### Step 3: tert-butyl 3-(2-(methoxycarbonyl)pyridin-4-yl)-5,6-dihydro-[1,2,4]triazolo[4,3-a]pyrazine-7(8H)-carboxylate

To a solution of tert-butyl 3-(2-bromo-4-pyridyl)-6,8-dihydro-5H-[1,2,4]triazolo[4,3-a]pyrazine-7-carboxylate (350 mg, 920.47 μmol, 1 eq) in MeOH (10 mL) was added Pd(dppf)Cl₂ (67.35 mg, 92.05 μmol, 0.1 eq) and TEA (279.43 mg, 2.76 mmol, 384.35 μL, 3 eq) under N₂ atmosphere. The suspension was degassed and purged with CO for 3 times. The mixture was stirred under CO (50 Psi or atm.) at 80° C. for 12 h. The reaction mixture was filtered and the filtrate was concentrated under the reduced pressure to give the product. The residue was purified by prep-TLC (SiO₂, DCM:MeOH=15:1, Rf=0.30) to give compound tert-butyl 3-(2-methoxycarbonyl-4-pyridyl)-6,8-dihydro-5H-[1,2,4]triazolo[4,3-a]pyrazine-7-carboxylate (280 mg, 76% yield) as a brown solid.

LC-MS [ESI, M+1]: 360.1.

¹H NMR (400 MHz, CHLOROFORM-d) δ=8.92 (d, J=4.8 Hz, 1H), 8.43 (s, 1H), 7.96 (br d, J=2.0 Hz, 1H), 4.96 (s, 2H), 4.25 (br s, 2H), 4.06 (s, 3H), 3.92 (br s, 2H), 1.53 (s, 9H).

### Step 4: methyl 4-(5,6,7,8-tetrahydro-[1,2,4]triazolo[4,3-a]pyrazin-3-yl)picolinate

To a solution of tert-butyl 3-(2-methoxycarbonyl-4-pyridyl)-6,8-dihydro-5H-[1,2,4]triazolo[4,3-a]pyrazine-7-carboxylate (100 mg, 278.26 μmol, 1 eq) in DCM (1 mL) was added TFA (767.50 mg, 6.73 mmol, 0.5 mL, 24.19 eq). The mixture was stirred at 25° C. for 0.5 h. LCMS showed starting material was consumed and one main peak with desired mass was detected. The reaction mixture was concentrated under the reduced pressure to give compound methyl 4-(5,6,7,8-tetrahydro-[1,2,4]triazolo[4,3-a]pyrazin-3-yl)pyridine-2-carboxylate (100 mg, crude, TFA) as a brown oil.

LC-MS [ESI, M+1]: 260.0.

### Step 5: methyl (R)-4-(7-(8-methyl-6-(3-methyl-3-phenylpyrrolidin-1-yl)-[1,2,4]triazolo[1,5-a]pyridine-2-carbonyl)-5,6,7,8-tetrahydro-[1,2,4]triazolo[4,3-a]pyrazin-3-yl)picolinate

To a solution of 8-methyl-6-[(3R)-3-methyl-3-phenyl-pyrrolidin-1-yl]-[1,2,4]triazolo[1,5-a]pyridine-2-carboxylic acid (50 mg, 148.64 μmol, 1 eq) in DMF (1 mL) was added methyl 4-(5,6,7,8-tetrahydro-[1,2,4]triazolo[4,3-a]pyrazin-3-yl)pyridine-2-carboxylate (99.87 mg, 267.55 μmol, 1.8 eq, TFA),methyl 4-(5,6,7,8-tetrahydro-[1,2,4]triazolo[4,3-a]pyrazin-3-yl)pyridine-2-carboxylate (99.87 mg, 267.55 μmol, 1.8 eq, TFA) HATU (84.78 mg, 222.96 μmol, 1.5 eq) and DIEA (57.63 mg, 445.92 μmol, 77.67 μL, 3 eq). The mixture was stirred at 25° C. for 1 h. LCMS showed starting material was consumed and one main peak with desired mass was detected. The reaction mixture was concentrated under the reduced pressure to give the crude product. The residue was purified by prep-HPLC (FA condition column: CD01-Phenomenex luna C18 150*25*10 um; mobile phase: [water(FA)—ACN]; gradient:32%-62% B over 8 min) to give compound methyl 4-[7-[8-methyl-6-[(3R)-3-methyl-3-phenyl-pyrrolidin-1-yl]-[1,2,4]triazolo[1,5-a]pyridine-2-carbonyl]-6,8-dihydro-5H-[1,2,4]triazolo[4,3-a]pyrazin-3-yl]pyridine-2-carboxylate (55 mg, 64% yield) as a yellow solid.

LC-MS [ESI, M+1]: 578.2.

¹H NMR (400 MHz, CHLOROFORM-d) δ=8.94 (br d, J=4.4 Hz, 1H), 8.47 (br s, 1H), 8.12-7.84 (m, 1H), 7.77-7.54 (m, 1H), 7.42-7.27 (m, 5H), 7.06 (s, 1H), 5.78 (br s, 1H), 5.33 (br s, 1H), 4.70-4.20 (m, 4H), 4.07 (s, 3H), 3.64 (br d, J=8.8 Hz, 1H), 3.60-3.49 (m, 2H), 3.44 (br s, 1H), 2.67 (s, 3H), 2.49-2.35 (m, 1H), 2.33-2.22 (m, 1H), 1.49 (s, 3H).

### Step 6: (R)-4-(7-(8-methyl-6-(3-methyl-3-phenylpyrrolidin-1-yl)-[1,2,4]triazolo[1,5-a]pyridine-2-carbonyl)-5,6,7,8-tetrahydro-[1,2,4]triazolo[4,3-a]pyrazin-3-yl)picolinic acid

To a solution of methyl 4-[7-[8-methyl-6-[(3R)-3-methyl-3-phenyl-pyrrolidin-1-yl]-[1,2,4]triazolo[1,5-a]pyridine-2-carbonyl]-6,8-dihydro-5H-[1,2,4]triazolo[4,3-a]pyrazin-3-yl]pyridine-2-carboxylate (50 mg, 86.56 μmol, 1 eq) in DCM (1 mL) was added hydroxy(trimethyl)stannane (156.52 mg, 865.60 μmol, 10 eq). The mixture was stirred at 25° C. for 0.5 h. LCMS showed starting material was consumed and one main peak with desired mass was detected. The reaction mixture was concentrated under the reduced pressure to give the crude product. The residue was purified by prep-HPLC (neutral condition column: CD02-Waters Xbidge BEH C18 150*25*10 um; mobile phase: [water(NH₄HCO₃)—ACN]; gradient:16%-46% B over 10 m) to give e compound 4-[7-[8-methyl-6-[(3R)-3-methyl-3-phenyl-pyrrolidin-1-yl]-[1,2,4]triazolo[1, 5-a]pyri dine-2-carb onyl]-6,8-dihydro-5H-[1,2,4]triazolo[4,3-a]pyrazin-3-yl]pyridine-2-carboxylic acid (12.09 mg, 2400 yield) as a white solid.

LC-MS [ESI, M+1]: 564.2.

¹H NMR (400 MHz, CHLOROFORM-d) δ=8.82 (br d, J=3.2 Hz, 1H), 8.48 (br d, J=16.8 Hz, 1H), 8.32-8.00 (m, 1H), 7.70 (br d, J=10.8 Hz, 1H), 7.44-7.32 (m, 4H), 7.32-7.29 (m, 1H), 7.06 (s, 1H), 5.78 (br s, 1H), 5.33 (br s, 1H), 4.70-4.51 (m, 2H), 4.49-4.32 (−, 2H), 3.65 (br d, J=8.8 Hz, 1H), 3.61-3.50 (m, 2H), 3.45 (br s, 1H), 2.68 (s, 3H), 2.49-2.36 (m, 1H), 2.35-2.234 (m, 1H), 1.49 (s, 3H).

| Cmpd. No. | Synthesis | LC/MS | ¹H NMR |
|---|---|---|---|
| 402 | Similar to | m/z 578.2 | ¹H NMR (400 MHz, CHLOROFORM-d) δ = 8.95 |
| | Example 36 | [M + H]⁺ | (br d, J = 5.2 Hz, 1H), 8.41 (d, J = 1.6 Hz, 1H), |
| | | | 8.02-7.79 (m, 1H), 7.75-7.61 (m, 1H), 7.42-7.27 (m, |
| | | | 5H), 7.04 (s, 1H), 5.37 (s, 1H), 5.13 (s, 1H), 4.31- |
| | | | 4.17 (m, 2H), 4.08 (s, 3H), 3.63 (br d, J = 8.8 Hz, |
| | | | 1H), 3.60-3.48 (m, 2H), 3.48-3.40 (m, 1H), 3.33- |
| | | | 3.19 (m, 2H), 2.70-2.64 (m, 3H), 2.46-2.36 (m, |
| | | | 1H), 2.33-2.24 (m, 1H), 1.49 (s, 3H) |
| 403 | Similar to | m/z 578.2 | ¹H NMR (400 MHz, CHLOROFORM-d) δ = 8.39- |
| | Example 36 | [M + H]+ | 8.20 (m, 1H), 8.12 (br d, J = 6.4 Hz, 1H), 7.92 (br |
| | | | d, J = 7.2 Hz, 1H), 7.75-7.58 (m, 1H), 7.44-7.28 |
| | | | (m, 4H), 7.27 (br s, 1H), 7.04 (br s, 1H), 5.67 (br s, |
| | | | 1H), 5.22 (br s, 1H), 4.78-4.30 (m, 4H), 3.98 (br s, |
| | | | 3H), 3.71-3.37 (m, 4H), 2.65 (br s, 3H), 2.46-2.35 |
| | | | (m, 1H), 2.29 (br d, J = 4.4 Hz, 1H), 1.48 (br s, 3H) |
| 404 | Similar to | m/z 564.2 | ¹H NMR (400 MHz, CHLOROFORM-d) δ = 8.49-8.15 |
| | Example 36 | [M + H]+ | (m, 2H), 8.03 (br s, 1H), 7.67 (br s, 1H), 7.41- |
| | | | 7.30 (m, 4H), 7.26-7.21 (m, 1H), 7.05 (br s, 1H), |
| | | | 5.65 (br s, 1H), 5.23 (br d, J = 2.0 Hz, 1H), 4.79- |
| | | | 4.32 (m, 4H), 3.63 (br d, J = 7.6 Hz, 1H), 3.55 (br |
| | | | dd, J = 1.2, 8.9 Hz, 2H), 3.43 (br s, 1H), 2.66 |
| | | | (br s, 3H), 2.45-2.37 (m, 1H), 2.33-2.25 (m, 1H), |
| | | | 1.48 (br s, 3H) |
| 405 | Similar to | m/z 564.2 | ¹H NMR (400 MHz, CHLOROFORM-d) δ = 8.96- |
| | Example 36 | [M + H]+ | 8.82 (m, 1H), 8.74-8.63 (m, 1H), 8.30-8.14 (m, |
| | | | 1H), 7.67 (br s, 1H), 7.42-7.35 (m, 2H), 7.35-7.30 |
| | | | (m, 2H), 7.25 (br s, 1H), 7.05 (s, 1H), 5.62 (s, 1H), |
| | | | 5.21 (s, 1H), 4.65 (br s, 1H), 4.53 (br s, 1H), 4.44 |
| | | | (br s, 2H), 3.63 (d, J = 8.8 Hz, 1H), 3.59-3.48 (m, |
| | | | 2H), 3.48-3.40 (m, 1H), 2.67 (s, 3H), 2.45-2.36 |
| | | | (m, 1H), 2.33-2.25 (m, 1H), 1.48 (s, 3H) |
| 406 | Similar to | m/z 578.1 | ¹H NMR (400 MHz, CHLOROFORM-d) δ = 8.61- |
| | Example 36 | [M + H]⁺ | 8.45 (m, 1H), 8.22-8.10 (m, 1H), 8.05-7.94 (m, |
| | | | 1H), 7.75-7.63 (m, 1H), 7.42-7.35 (m, 2H), 7.35- |
| | | | 7.30 (m, 2H), 7.29 (br s, 1H), 7.04 (s, 1H), 5.72 (s, |
| | | | 1H), 5.32 (s, 1H), 5.05-4.79 (m, 2H), 4.66-4.23 |
| | | | (m, 2H), 4.09-3.97 (m, 3H), 3.63 (d, J = 8.8 Hz, |
| | | | 1H), 3.60-3.48 (m, 2H), 3.48-3.38 (m, 1H), 2.67 |
| | | | (s, 3H), 2.40 (td, J = 8.4, 12.2 Hz, 1H), |
| | | | 2.32-2.24 (m, 1H), 1.48 (s, 3H) |
| 407 | Similar to | m/z 564.2 | ¹H NMR (400 MHz, DMSO-d₆) δ = 8.86 (br s, 1H), |
| | Example 36 | [M + H]⁺ | 8.39 (br d, J = 7.6 Hz, 1H), 7.98 (br s, 2H), 7.43- |
| | | | 7.32 (m, 5H), 7.24 (br t, J = 7.2 Hz, 1H), 5.44 (s, |
| | | | 1H), 5.13 (s, 1H), 4.40 (br s, 2H), 4.34-4.11 (m, |
| | | | 2H), 3.66-3.47 (m, 3H), 3.40 (br d, J = 1.6 Hz, 1H), |
| | | | 2.56 (s, 3H), 2.27 (br t, J = 6.8 Hz, 2H), 1.36 (s, 3H) |
| 408 | Similar to | m/z 564.1 | ¹H NMR (400 MHz, CHLOROFORM-d) δ = 8.62- |
| | Example 36 | [M + H]⁺ | 8.38 (m, 1H), 8.30-7.93 (m, 2H), 7.68 (br s, 1H), |
| | | | 7.45-7.30 (m, 4H), 7.30-7.27 (m, 1H), 7.05 (br s, |
| | | | 1H), 5.78 (br s, 1H), 5.35 (br s, 1H), 4.87 (br s, 2H), |
| | | | 4.67-4.49 (m, 1H), 4.38 (br s, 1H), 3.63 (br d, J = |
| | | | 8.0 Hz, 1H), 3.59-3.48 (m, 2H), 3.43 (br s, 1H), |
| | | | 2.66 (br s, 3H), 2.48-2.21 (m, 2H), 1.48 (br s, 3H) |

### Example 37

### 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid (Compound 37)

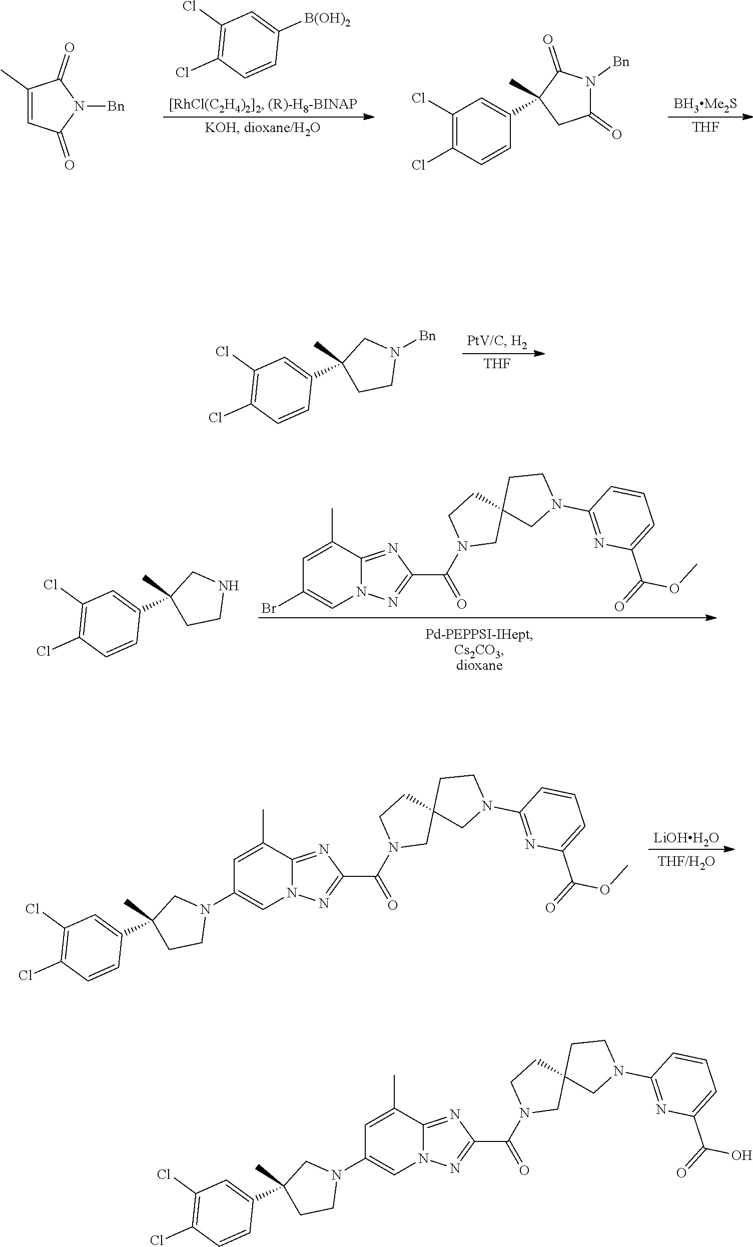

### Step 1: (3R)-1-benzyl-3-(3,4-dichlorophenyl)-3-methyl-pyrrolidine-2,5-dione

Solution A: To a mixture of chlororhodium ethylene (77.31 mg, 198.79 μmol, 0.01 eq) (R)-(+)-2,2′-BIS(DIPHENYLPHOSPHINO)-1,1′-BINAPHTHYL (123.78 mg, 198.79 μmol, 0.01 eq) in dioxane (250 mL)(100 mL) was stirred at 25° C. for 15 min, then the mixture was added (3,4-dichlorophenyl)boronic acid (11.38 g, 59.64 mmol, 3 eq), KOH (1 M, 9.94 mL, 0.5 eq) was stirred at 25° C. for 3 min. Solution B: To a mixture of 1-benzyl-3-methyl-pyrrole-2,5-dione (4 g, 19.88 mmol, 1 eq) in dioxane (150 mL) then pure solution A to solution B stirred at 60° C. for 1 h. LCMS showed starting material was consumed and one main peak with desired mass was detected. The reaction mixture was extracted with EtOAc (300 mL×3), The combined organic layers were dried over anhydrous Na2SO4, filtered and dried to give a residue. The residue was purified by prep-TLC (SiO₂, PE:EtOAc (NH₃·MeOH 1%)=1:1, Rf=0.4) and eluted with 60 mL (10% methanol in DCM) to give compound (3R)-1-benzyl-3-(3,4-dichlorophenyl)-3-methyl-pyrrolidine-2,5-dione (2.5 g, 36% yield) was obtained as a white solid.

LC-MS [ESI, M+1]: 347.9

¹H NMR (CHLOROFORM-d, 400 MHz): δ=7.26-7.51 (m, 7.5H), 7.13 (br d, J=8.4 Hz, 1.0H), 4.74 (s, 2.4H), 2.96-3.18 (m, 1.1H), 2.78-2.92 (m, 1.0H), 1.69 ppm (s, 3.1H).

### Step 2: (3R)-1-benzyl-3-(3,4-dichlorophenyl)-3-methyl-pyrrolidine

To a solution of (3R)-1-benzyl-3-(3,4-dichlorophenyl)-3-methyl-pyrrolidine-2,5-dione (1.15 g, 3.30 mmol, 1 eq) in THF (15 mL) was added BH₃-Me₂S (10 M, 990.75 μL, 3 eq) at 0° C. for 5 min under N₂ atmosphere. The mixture was stirred at 70° C. for 1 h. LCMS showed starting material was consumed and one main peak with desired mass was detected. The mixture was concentrated in vacuo. The residue was purified by flash silica gel chromatography (ISCO®; 4 g SepaFlash® Silica Flash Column, Eluent of 0~50% PE/EtOAc @40 mL/min), TLC (PE:EtOAc)=1:1, Rf=0.5) to give compound (3R)-1-benzyl-3-(3,4-dichlorophenyl)-3-methyl-pyrrolidine (580 mg, 55% yield) was obtained as a white gum

LC-MS [ESI, M+1]: 320.1

¹H NMR (CHLOROFORM-d, 400 MHz): δ=7.33-7.44 (m, 2.1H), 7.28-7.32 (m, 3.2H), 7.25 (br s, 0.9H), 7.15-7.23 (m, 2.2H), 7.05-7.13 (m, 1.4H), 3.76-4.27 (m, 1.0H), 3.45-3.75 (m, 3.0H), 2.63-2.78 (m, 3.3H), 2.07 (ddd, J=12.4, 8.2, 7.2 Hz, 1.1H), 1.89 (ddd, J=12.4, 7.2, 5.2 Hz, 1.3H), 1.36 ppm (s, 3.0H).

### Step 3: (3R)-3-(3,4-dichlorophenyl)-3-methyl-pyrrolidine

To a solution of (3R)-1-benzyl-3-(3,4-dichlorophenyl)-3-methyl-pyrrolidine (700 mg, 2.19 mmol, 1 eq) in Tol. (5 mL) was added 1-chloroethyl carbonochloridate (468.74 mg, 3.28 mmol, 1.5 eq). The mixture was stirred at 100° C. for 1 h. LCMS showed starting material was consumed and one main peak with desired mass was detected. The mixture was filtered. The residue was purified by flash silica gel chromatography (ISCO®; 4 g SepaFlash® Silica Flash Column, Eluent of 0~80% EA:PE @40 mL/min), TLC (DCM:MeOH)=10:1, Rf=0.2) to give compound (3R)-3-(3,4-dichlorophenyl)-3-methyl-pyrrolidine (450 mg, 89% yield) was obtained as a yellow gum.

LC-MS [ESI, M+1]: 229.9.

¹H NMR (CHLOROFORM-d, 400 MHz): δ=7.33-7.40 (m, 1.2H), 7.27-7.32 (m, 1.2H), 7.02-7.10 (m, 1.2H), 3.18-3.48 (m, 4.1H), 1.96-2.27 (m, 2.0H), 1.40 ppm (s, 3.0H).

### Step 4: 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid

To a solution of (3R)-3-(3,4-dichlorophenyl)-3-methyl-pyrrolidine (55.30 mg, 240.31 μmol, 2 eq) and methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (60 mg, 120.15 μmol, 1 eq) in dioxane (4 mL) was added 1,3-bis[2,6-bis(1-propylbutyl)phenyl]-4,5-dichloro-2H-imidazol-1-ium-2-ide;3-chloropyridine;dichloropalladium (5.84 mg, 6.01 μmol, 0.05 eq) and Cs₂CO₃ (117.45 mg, 360.46 μmol, 3 eq).The mixture was stirred at 100° C. for 1 h. LCMS showed starting material was consumed and one main peak with desired mass was detected. The mixture was quenched with water (0.1 mL) and then filtered. The residue was purified by prep-HPLC (column: CD04-Welch Ultimate C18 150*25*7 um; mobile phase: [water(FA)—ACN]; gradient:60%-90% B over 10 min) and lyophilized to give compound 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid (15 mg, 20% yield) was obtained as a white gum.

LC-MS [ESI, M+1]: 648.3.

### Step 5: 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylate (15 mg, 23.13 μmol, 1 eq) in THF (1 mL) and H₂O (0.2 mL) was added LiOH·H₂O (2.91 mg, 69.38 μmol, 3 eq) The mixture was stirred at 25° C. for 30 min. LCMS showed starting material was consumed and one main peak with desired mass was detected. The reaction mixture was concentrated under the reduced pressure to give the product. The residue was purified by prep-HPLC (column: CD01-Phenomenex luna C18 150*25*10 um; mobile phase: [water(FA)—ACN]; gradient:36%-66% B over 11 min) and lyophilized to give compound 6-[(5R)-2-[6-[(3R)-3-(3,4-dichlorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid (4.27 mg, 2900 yield) was obtained as a white solid.

LC-MS [ESI, M+1]: 634.2.

¹H NMR (CHLOROFORM-d, 400 MHz) δ=7.60-7.73 (in, 2.0H), 7.48-7.55 (−, 1.0H), 7.38-7.48 (m, 1.9H), 7.12-7.20 ((, 1.0H), 7.00 (br d, J=9.6 Hz, 1.0H), 6.64 (t, J=8.4 Hz, 1.0H), 4.22 (br d, J=6.4 Hz, 1.0H), 4.03 (s, 1.0H), 3.93 (s, 1.4H), 3.76-3.89 (m, 1.2H), 3.62-3.75 (in, 2.1H), 3.46-3.62 (m, 5.2H), 3.39-3.46 (m, 1.1H), 2.66 (d, J=11.2 Hz, 3.0H), 2.30-2.39 (m, 1.0H), 2.23-2.29 (m, 1.1H), 2.03-2.18 (m, 4.0H), 1.47 ppm (d, J=8.0 Hz, 3H).

| Cmpd. No. | Synthesis | LC/MS | ¹H NMR |
|---|---|---|---|
| 409 | Similar to | m/z 596.2 | ¹H NMR (400 MHz, CHLOROFORM-d) δ = 7.73- |
| | Example 37 | [M + H]⁺ | 7.58 (m, 2H), 7.48 (t, J = 6.8 Hz, 1H), 7.33-7.27 |
| | | | (m, 1H), 6.99 (br d, J = 9.6 Hz, 1H), 6.90 (t, J = 7.2 |
| | | | Hz, 1H), 6.86 (td, J = 2.0, 7.1 Hz, 1H), 6.83-6.76 |
| | | | (m, 1H), 6.62 (t, J = 8.4 Hz, 1H), 4.28-4.13 (m, |
| | | | 1H), 4.02 (s, 1H), 3.92 (br t, J = 7.2 Hz, 1H), |
| | | | 3.88-3.71 (m, 4H), 3.69-3.60 (m, 2H), 3.60-3.53 (m, |
| | | | 2H), 3.53-3.47 (m, 2H), 3.47-3.35 (m, 2H), 2.64 (d, |
| | | | J = 10.9 Hz, 3H), 2.37 (qd, J = 8.0, 12.1 Hz, |
| | | | 1H), 2.28-2.20 (m, 1H), 2.20-1.98 (m, 4H), 1.45 (d, |
| | | | J = 7.6 Hz, 3H) |
| 410 | Similar to | m/z 582.3 | ¹H NMR (400 MHz, DMSO-d₆) δ = 9.52 (br s, 1H), |
| | Example 37 | [M + H]⁺ | 7.82 (br d, J = 12.4 Hz, 1H), 7.62 (td, J = 8.0, |
| | | | 12.0 Hz, 1H), 7.28-7.19 (m, 2H), 7.19-7.13 (m, 1H), |
| | | | 7.10-7.02 (m, 1H), 6.84 (dd, J = 4.4, 7.6 Hz, 1H), |
| | | | 6.76 (q, J = 6.8 Hz, 1H), 6.66 (dd, J = 8.4, 17.2 Hz, |
| | | | 1H), 3.96 (br t, J = 7.2 Hz, 1H), 3.87-3.74 (m, 1H), |
| | | | 3.74-3.65 (m, 2H), 3.63-3.41 (m, 8H), 2.54 (s, |
| | | | 3H), 2.38-2.27 (m, 2H), 2.11-1.86 (m, 4H), 1.34 |
| | | | (d, J = 8.4 Hz, 3H) |
| 411 | Similar to | m/z 596.3 | ¹H NMR (400 MHz, CHLOROFORM-d) δ = 7.70- |
| | Example 37 | [M + H]⁺ | 7.59 (m, 2H), 7.48 (t, J = 6.4 Hz, 1H), 7.23 (dd, J = |
| | | | 7.2, 8.8 Hz, 2H), 6.99 (br d, J = 9.6 Hz, 1H), 6.93- |
| | | | 6.85 (m, 2H), 6.62 (t, J = 8.4 Hz, 1H), 4.27-4.14 |
| | | | (m, 1H), 4.02 (s, 1H), 3.92 (br t, J = 7.2 Hz, 1H), |
| | | | 3.87-3.73 (m, 4H), 3.70-3.37 (m, 8H), 2.64 (br d, |
| | | | J = 10.8 Hz, 3H), 2.40-2.29 (m, 1H), 2.27-2.19 |
| | | | (m, 1H), 2.18-1.98 (m, 4H), 1.44 (d, J = 7.6 Hz, |
| | | | 3H) |
| 412 | Similar to | m/z 579.3 | ¹H NMR (CHLOROFORM-d, 400 MHz): δ = 8.07 |
| | Example 37 | [M + H]⁺ | (s, 0.2H), 7.81 (br s, 1.0H), 7.66 (br d, J = 4.8 Hz, |
| | | | 1.1H), 7.48 (br s, 1.0H), 7.42 (br t, J = 5.6 Hz, 1.0H), |
| | | | 7.09-7.25 (m, 3.0H), 6.64 (br d, J = 6.4 Hz, 1.0H), |
| | | | 5.19 (br d, J = 6.2 Hz, 1.0H), 4.17 (br s, 1.1H), |
| | | | 3.98 (br s, 1.1H), 3.74-3.93 (m, 2.1H), 3.47-3.70 (m, |
| | | | 4.1H), 2.61 (br d, J = 12.8 Hz, 3.0H), 2.03-2.17 (m, |
| | | | 4.0H), 1.67 ppm (br t, J = 6.4 Hz, 3.0H) |
| 413 | Similar to | m/z 582.3 | ¹H NMR (400 MHz, DMSO-d₆) δ = 9.41-9.10 (m, |
| | Example 37 | [M + H]⁺ | 1H), 7.96-7.78 (m, 1H), 7.69-7.55 (m, 1H), 7.33- |
| | | | 7.20 (m, 2H), 7.19-7.09 (m, 2H), 6.77-6.63 (m, |
| | | | 3H), 3.97 (br t, J = 7.2 Hz, 1H), 3.81 (br d, J = 2.0 |
| | | | Hz, 1H), 3.73-3.42 (m, 10H), 2.61-2.52 (m, 3H), |
| | | | 2.25-2.09 (m, 2H), 2.06-1.90 (m, 4H), 1.31 (br d, |
| | | | J = 8.8 Hz, 3H) |
| 414 | Similar to | m/z 566.3 | ¹H NMR (400 MHz, CHLOROFORM-d) δ ppm |
| | Example 37 | [M + H]⁺ | 1.45 (s, 3 H) 2.20-2.28 (m, 1 H) 2.32-2.40 (m, 4 |
| | | | H) 2.64 (s, 3 H) 3.12-3.24 (m, 2 H) 3.40 (td, J = 8.8, |
| | | | 3.75 Hz, 1 H) 3.45 (d, J = 8.8 Hz, 1 H) 3.48-3.60 |
| | | | (m, 4 H) 3.75-3.88 (m, 3 H) 3.98-4.14 (m, 2 H) 4.39 |
| | | | (br dd, J = 11.4, 7.13 Hz, 1 H) 6.63 (d, J = 8.4 Hz, |
| | | | 1 H) 6.99 (s, 1 H) 7.13-7.24 (m, 4 H) 7.50 (d, J = 7.2 |
| | | | Hz, 1 H) 7.60-7.71 (m, 2 H) |
| 415 | Similar to | m/z 566.5 | ¹H NMR (400 MHz, CHLOROFORM-d) δ = 7.72- |
| | Example 37 | [M + H]⁺ | 7.60 (m, 2H), 7.50 (d, J = 7.2 Hz, 1H), 7.26-7.22 |
| | | | (m, 1H), 7.15-7.05 (m, 3H), 7.00 (s, 1H), 6.64 (d, |
| | | | J = 8.4 Hz, 1H), 4.44-4.34 (m, 1H), 4.12-4.01 (m, |
| | | | 2H), 3.88-3.74 (m, 3H), 3.62-3.45 (m, 5H), 3.41 |
| | | | (dt, J = 3.2, 8.7 Hz, 1H), 3.23-3.13 (m, 2H), 2.64 |
| | | | (s, 3H), 2.43-2.32 (m, 4H), 2.28-2.22 (m, 1H), |
| | | | 1.44 (s, 3H) |
| 416 | Similar to | m/z 580.2 | ¹H NMR (CHLOROFORM-d, 400 MHz): δ = |
| | Example 37 | [M + H]⁺ | 7.56-7.76 (m, 1.9H), 7.49 (br t, J = 6.4 Hz, 1.0H), |
| | | | 7.22-7.26 (m, 1.2H), 7.03-7.17 (m, 2.9H), 7.00 (br d, |
| | | | J = 9.6 Hz, 1.0H), 6.62 (t, J = 8.4 Hz, 1.0H), 4.21 |
| | | | (br d, J = 4.8 Hz, 1.0H), 4.02 (s, 1.0H), 3.92 (br s, |
| | | | 1.0H), 3.74-3.88 (m, 1.2H), 3.35-3.73 (m, 8.0H), 2.65 |
| | | | (br d, J = 10.8 Hz, 3.1H), 2.38 (br d, J = 4.4 Hz, |
| | | | 3.2H), 1.99-2.30 (m, 6.0H), 1.45 ppm (br d, J = 7.6 |
| | | | Hz, 3.0H) |
| 417 | Similar to | m/z 600.2 | ¹H NMR (400 MHz, METHANOL-d4) δ = 7.85 (d, |
| | Example 37 | [M + H]⁺ | J = 7.6 Hz, 1H), 7.57-7.48 (m, 1H), 7.48-7.40 (m, |
| | | | 2H), 7.36-7.21 (m, 4H), 6.57-6.48 (m, 1H), 4.22- |
| | | | 4.16 (m, 1H), 4.05 (s, 1H), 3.99-3.94 (m, 1H), |
| | | | 3.89-3.85 (m, 1H), 3.77-3.58 (m, 9H), 2.62 (d, |
| | | | J = 10.8 Hz, 3H), 2.59-2.54 (m, 1H), 2.19-2.04 |
| | | | (m, 4H), 1.52 (d, J = 8.4 Hz, 3H) |
| 418 | Similar to | m/z 586.4 | ¹H NMR (400 MHz, METHANOL-d4) δ = 7.87- |
| | Example 37 | [M + H]⁺ | 7.65 (m, 2H), 7.53-7.18 (m, 6H), 6.80 (d, J = 8.8 Hz, |
| | | | 1H), 4.39-4.26 (m, 1H), 4.07-3.98 (m, 2H), 3.95 |
| | | | (d, J = 9.6 Hz, 1H), 3.91-3.79 (m, 2H), 3.77-3.69 |
| | | | (m, 1H), 3.65-3.43 (m, 6H), 3.24-3.21 (m, 1H), |
| | | | 2.69-2.42 (m, 5H), 1.50 (s, 3H) |
| 419 | Similar to | m/z 586.3 | ¹H NMR (400 MHz, DMSO-d6) δ = 7.94 (s, 1H), |
| | Example 37 | [M + H]⁺ | 7.60 (t, J = 8.0 Hz, 1H), 7.47-7.44 (m, 1H), 7.42- |
| | | | 7.34 (m, 2H), 7.33-7.28 (m, 2H), 7.20 (d, J = 7.2 |
| | | | Hz, 1H), 6.62 (d, J = 8.4 Hz, 1H), 4.19-4.01 (m, |
| | | | 1H), 3.88-3.77 (m, 2H), 3.77-3.66 (m, 2H), 3.66- |
| | | | 3.59 (m, 2H), 3.57-3.52 (m, 1H), 3.49 (d, J = 9.2 |
| | | | Hz, 2H), 3.47-3.42 (m, 1H), 3.39 (d, J = 10.4 Hz, |
| | | | 3H), 3.12-3.05 (m, 2H), 2.53 (s, 3H), 2.29-2.19 |
| | | | (m, 2H), 1.34 (s, 3H) |
| 420 | Similar to | m/z 586.3 | ¹H NMR (400 MHz, METHANOL-d4) δ = 7.87- |
| | Example 37 | [M + H]⁺ | 7.67 (m, 2H), 7.47-7.20 (m, 6H), 6.82 (d, J = 8.4 |
| | | | Hz, 1H), 4.36-4.27 (m, 1H), 4.08-3.98 (m, 2H), |
| | | | 3.91-3.78 (m, 2H), 3.76-3.70 (m, 1H), 3.63-3.53 |
| | | | (m, 5H), 3.45-3.41 (m, 1H), 3.27-3.18 (m, 2H), |
| | | | 2.59 (s, 3H), 2.40-2.24 (m, 2H), 1.44 (s, 3H) |
| 421 | Similar to | m/z 588.4 | ¹H NMR (400 MHz, DMSO-d6) δ = 7.92 (s, 1H), |
| | Example 37 | [M + H]⁺ | 7.64-7.57 (m, 1H), 7.51-7.44 (m, 1H), 7.43-7.36 |
| | | | (m, 1H), 7.28 (s, 1H), 7.21 (d, J = 7.2Hz, 2H), |
| | | | 6.66-6.60 (m, 1H), 4.14-4.02 (m, 1H), 3.88-3.63 (m, |
| | | | 5H), 3.59-3.52 (m, 3H), 3.49 (d, J = 10.0 Hz, 3H), |
| | | | 3.08 (d, J = 7.2 Hz, 2H), 2.52 (s, 3H), 2.26-2.20 |
| | | | (m, 2H), 1.34 (s, 3H) |
| 422 | Similar to | m/z 558.3 | ¹H NMR (400 MHz, METHANOL-d4) δ = 8.07 (d, |
| | Example 37 | [M + H]⁺ | J = 7.6 Hz, 1H), 7.81 (d, J = 1.6 Hz, 1H), 7.71 (d, |
| | | | J = 3.2 Hz, 1H), 7.46-7.32 (m, 2H), 7.27 (s, 1H), |
| | | | 7.25-7.20 (m, 1H), 7.12-7.02 (m, 2H), 4.42-4.31 |
| | | | (m, 3H), 3.61-3.52 (m, 3H), 3.51-3.40 (m, 3H), |
| | | | 2.60 (s, 3H), 2.41-2.25 (m, 2H), 2.25-2.17 (m, |
| | | | 2H), 1.86-1.74 (m, 2H), 1.43 (s, 3H) |
| 423 | Similar to | m/z 585.4 | ¹H NMR (400 MHz, METHANOL-d4) δ = 8.41 (m, |
| | Example 37 | [M + H]⁺ | J = 3.0, 5.6 Hz, 1H), 7.84-7.67 (m, 2H), 7.63-7.47 |
| | | | (m, 2H), 7.41-7.19 (m, 2H), 6.83 (m, J = 8.8, 16.6 |
| | | | Hz, 1H), 4.23-4.11 (m, 1H), 4.09-3.98 (m, 1H), |
| | | | 3.90-3.79 (m, 2H), 3.77-3.45 (m, 7H), 3.44-3.37 |
| | | | (m, 1H), 2.63-2.49 (m, 4H), 2.33-2.05 (m, 5H), |
| | | | 1.49 (d, J = 8.4 Hz, 3H) |
| 424 | Similar to | m/z 585.3. | ¹H NMR (400 MHz, METHANOL-d4) δ = 8.41 (m, |
| | Example 37 | [M + H]⁺ | J = 3.0, 5.6 Hz, 1H), 7.84-7.67 (m, 2H), 7.63-7.47 |
| | | | (m, 2H), 7.41-7.19 (m, 2H), 6.83 (m, J = 8.8, 16.6 |
| | | | Hz, 1H), 4.23-4.11 (m, 1H), 4.09-3.98 (m, 1H), |
| | | | 3.90-3.79 (m, 2H), 3.77-3.45 (m, 7H), 3.44-3.37 |
| | | | (m, 1H), 2.63-2.49 (m, 4H), 2.33-2.05 (m, 5H), |
| | | | 1.49 (d, J = 8.4 Hz, 3H) |
| 425 | Similar to | m/z 368.3 | ¹H NMR (400 MHz, CD3OD, 296 K) δ (ppm) = |
| | Example 37 | [M + H]⁺ | 8.55-8.31 (m, 2H), 7.86 (d, J = 13.3 Hz, 1H), 7.79- |
| | | | 7.68 (m, 2H), 7.41-7.25 (m, 2H), 6.85 (m, J = 8.6, |
| | | | 17.0 Hz, 1H), 4.23-4.12 (m, 1H), 4.04 (m, J = 11.6 |
| | | | Hz, 1H), 3.90-3.81 (m, 1H), 3.79-3.54 (m, 8H), |
| | | | 3.50-3.41 (m, 1H), 2.60 (d, J = 13.6 Hz, 3H), 2.43- |
| | | | 2.30 (m, 2H), 2.26-2.02 (m, 4H), 1.50 (d, J = |
| | | | 8.4Hz, 3H) |
| 426 | Similar to | m/z 584.4 | ¹H NMR (400 MHz, CHLOROFORM-d) δ = 7.63 |
| | Example 37 | [M + H]⁺ | (s, 1H), 7.57 (s, 1H), 7.37-7.32 (m, 4H), 6.99 (s, |
| | | | 1H), 6.62 (s, 1H), 4.50-4.41 (m, 2H), 3.65-3.60 |
| | | | (m, 1H), 3.57-3.49 (m, 2H), 3.45-3.39 (m, 1H), |
| | | | 2.39-2.31 (m, 1H), 2.26-2.19 (m, 1H), 1.57 (s, |
| | | | 9H), 1.47-1.42 (m, 6H) |
| 427 | Similar to | m/z 567.3 | ¹H NMR (400 MHz, DMSO-d6) δ = 12.60 (s, 1H), |
| | Example 37 | [M + H]⁺ | 8.70-8.59 (m, 1H), 8.49-8.39 (m, 1H), 8.14 (s, |
| | | | 1H), 7.99-7.88 (m, 1H), 7.84-7.75 (m, 1H), 7.69- |
| | | | 7.56 (m, 1H), 7.42-7.34 (m, 1H), 7.33-7.19 (m, |
| | | | 2H), 6.73-6.62 (m, 1H), 3.96 (t, J = 7.2 Hz, 1H), |
| | | | 3.84-3.76 (m, 1H), 3.71-3.40 (m, 10H), 2.57- |
| | | | 2.51 (m, 3H), 2.34-2.27 (m, 2H), 2.08-1.92 (m, |
| | | | 4H), 1.38 (d, J = 9.2 Hz, 3H). |
| 428 | Similar to | m/z 584.2 | ¹H NMR (400 MHz, METHANOL-d4) δ = 7.87- |
| | Example 37 | [M + H]⁺ | 7.69 (m, 2H), 7.43-7.23 (m, 4H), 7.12-7.00 (m, |
| | | | 2H), 6.89-6.78 (m, 1H), 4.24-4.13 (m, 1H), 4.10- |
| | | | 4.00 (m, 1H), 3.86 (t, J = 6.8 Hz, 1H), 3.78-3.67 |
| | | | (m, 3H), 3.67-3.60 (m, 2H), 3.57 (d, J = 13.2 Hz, |
| | | | 3H), 3.46-3.38 (m, 2H), 2.59 (d, J = 13.8 Hz, 3H), |
| | | | 2.38-2.27 (m, 2H), 2.25-2.06 (m, 4H), 1.49-1.38 |
| | | | (m, 3H) |
| 429 | Similar to | m/z 584.2 | ¹H NMR (400 MHz, METHANOL-d4) δ = 7.84- |
| | Example 37 | [M + H]⁺ | 7.70 (m, 2H), 7.42-7.33 (m, 3H), 7.26 (d, J = 13.2 |
| | | | Hz, 1H), 7.12-7.01 (m, 2H), 6.91-6.79 (m, 1H), |
| | | | 4.23-4.13 (m, 1H), 4.10-4.00 (m, 1H), 3.86 (t, J = |
| | | | 7.6 Hz, 1H), 3.80-3.68 (m, 3H), 3.67-3.60 (m, |
| | | | 2H), 3.59-3.52 (m, 3H), 3.46-3.37 (m, 1H), 2.59 |
| | | | (d, J = 14.0 Hz, 3H), 2.37-2.27 (m, 2H), 2.23-2.07 |
| | | | (m, 4H), 1.43 (d, J = 9.2 Hz, 3H) |

### Example 38

### methyl 6-[5-[8-methyl-6-[3-methyl-3-(5-methyl-1,3,4-thiadiazol-2-yl)pyrrolidin-1-yl]-[1,2,4]triazolo[1,5-a]pyridine-2-carbothioyl]-1,3,3a, 4,6,6a-hexahydropyrrolo[3,4-c]pyrrol-2-yl]pyridine-2-carboxylate (Compound 38)

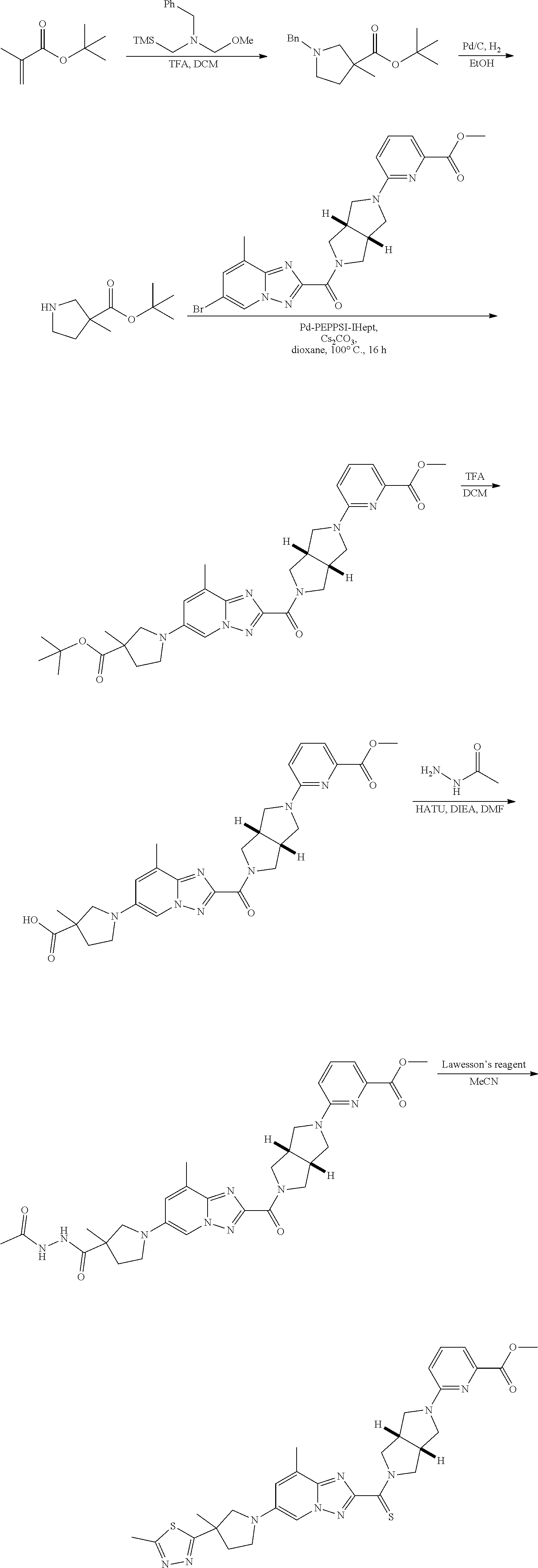

### Step 1: tert-butyl 1-benzyl-3-methyl-pyrrolidine-3-carboxylate

To a solution of tert-butyl 2-methylprop-2-enoate (10 g, 70.33 mmol, 1 eq) and N-(methoxymethyl)-1-phenyl-N-(trimethylsilylmethyl)methanamine (16.70 g, 70.33 mmol, 1 eq) in DCM (200 mL) was added TFA (1 M, 10 mL, 1.42e⁻¹ eq). The mixture was stirred at 25° C. for 2 h. TLC (DCM:MeOH=10:1) showed reactant was consumed and one main spot formed. The pH was adjusted to around 9 by progressively adding solid NaHCO₃. The reaction was added water (50 mL) and then extracted with DCM (50 mL×3). The combined organic phase was washed with brine (100 mL), dried over anhydrous Na2SO4, filtered and concentrated in vacuo. The residue was purified by column chromatography (SiO₂, DCM:MeOH=10:1, Rf=0.3) to give compound tert-butyl 1-benzyl-3-methyl-pyrrolidine-3-carboxylate (18 g, 84% yield) as yellow oil.

¹H NMR (400 MHz, CHLOROFORM-d) δ ppm 1.32 (s, 3H) 1.46 (s, 9H) 1.62 (dt, J=12.8, 7.6 Hz, 1H) 2.32-2.45 (m, 2H) 2.55-2.65 (m, 1H) 2.66-2.76 (m, 1H) 2.90 (d, J=9.2 Hz, 1H) 3.57-3.69 (m, 2H) 7.20-7.27 (m, 1H) 7.29-7.37 (m, 4H).

### Step 2: tert-butyl 3-methylpyrrolidine-3-carboxylate

To a solution of tert-butyl 1-benzyl-3-methyl-pyrrolidine-3-carboxylate (8 g, 29.05 mmol, 1 eq) in THF (100 mL) was added Pd/C (3.09 g, 2.91 mmol, 10% purity, 0.1 eq) under N₂ atmosphere. The suspension was degassed and purged with H2 for 3 times. The mixture was stirred under H2 (50 Psi) at 50° C. for 12 h. LCMS showed starting material was consumed and the desired mass was detected. The reaction mixture was filtered and the filtrate was concentrated under the reduced pressure to give the compound tert-butyl 3-methylpyrrolidine-3-carboxylate (4.6 g, 85% yield) as a brown oil.

LC-MS [ESI, M+1]: 186.2.

¹H NMR (400 MHz, CHLOROFORM-d) δ ppm 1.25 (s, 3H) 1.43 (s, 9H) 1.58 (ddd, J=13.2, 7.6, 6.0 Hz, 1H) 2.14-2.27 (m, 1H) 2.61 (d, J=11.6 Hz, 1H) 2.90-3.13 (m, 2H) 3.30 (d, J=11.6 Hz, 1H) 3.36 (br s, 1H).

### Step 3: methyl 6-[5-[6-(3-tert-butoxycarbonyl-3-methyl-pyrrolidin-1-yl)-8-methyl-[1,2,4]triazolo[1,5-a]pyridine2-carbonyl]-1,3,3a, 4,6,6a-hexahydropyrrolo[3,4-c]pyrrol-2-yl]pyridine-2-carboxylate

A mixture of methyl 6-[5-(6-bromo-8-methyl-[1,2,4]triazolo[1,5-a]pyridine-2-carbonyl)-1,3,3a, 4,6,6a-hexahydropyrrolo[3,4-c]pyrrol-2-yl]pyridine-2-carboxylate (2 g, 4.12 mmol, 1 eq), tert-butyl 3-methylpyrrolidine-3-carboxylate (992.48 mg, 5.36 mmol, 1.3 eq), 1,3-bis[2,6-bis(1-propylbutyl)phenyl]-4,5-dichloro-2H-imidazol-1-ium2-ide;3-chloropyridine;dichloropalladium (200.43 mg, 206.04 μmol, 0.05 eq), Cs₂CO₃ (4.03 g, 12.36 mmol, 3 eq) in dioxane (40 mL) was degassed and purged with N₂ for 3 times, and then the mixture was stirred at 100° C. for 12 h under N₂ atmosphere. LCMS showed starting material was consumed and the desired mass was detected. The reaction was filtered and the filtered concentrated under reduced pressure to give a residue. The residue was purified by column chromatography (SiO₂, DCM:MeOH=10:1, Rf=0.5) to give the compound methyl 6-[5-[6-(3-tert-butoxycarbonyl-3-methyl-pyrrolidin-1-yl)-8-methyl-[1,2,4]triazolo[1,5-a]pyridine2-carbonyl]-1,3,3a, 4,6,6a-hexahydropyrrolo[3,4-c]pyrrol-2-yl]pyridine-2-carboxylate (2.2 g, 82% yield) as a brown oil.

LC-MS [ESI, M+1]: 590.3.

¹H NMR (400 MHz, CHLOROFORM-d) δ ppm 1.37-1.42 (m, 3H) 1.44-1.45 (m, 9H) 1.93 (dt, J=12.8, 7.2 Hz, 1H) 2.49 (td, J=13.2, 7.2 Hz, 1H) 2.48-2.48 (m, 1H) 2.57-2.69 (m, 3H) 3.07-3.17 (m, 3H) 3.36 (s, 1H) 3.47-3.53 (m, 1H) 3.61 (br dd, J=10.8, 2.4 Hz, 1H) 3.68-3.74 (m, 2H) 3.77-3.85 (m, 3H) 3.93 (s, 3H) 3.98 (br dd, J=12.0, 4.8 Hz, 1H) 4.05 (br dd, J=12.8, 7.2 Hz, 1H) 4.30-4.38 (m, 1H) 6.50-6.56 (m, 1H) 6.93 (d, J=0.8 Hz, 1H) 7.37-7.40 (m, 1H) 7.52-7.56 (m, 1H) 7.56-7.60 (m, 1H).

### Step 4: 1-[2-[2-(6-methoxycarbonyl-2-pyridyl)-1,3,3a, 4,6,6a-hexahydropyrrolo[3,4-c]pyrrole5-carbonyl]-8-methyl-[1,2,4]triazolo[1,5-a]pyridin-6-yl]-3-methyl-pyrrolidine-3-carboxylic acid

To a solution of methyl 6-[5-[6-(3-tert-butoxycarbonyl-3-methyl-pyrrolidin-1-yl)-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate (500 mg, 847.91 μmol, 1 eq) in DCM (6 mL) was added TFA (4.61 g, 40.39 mmol, 3 mL, 47.63 eq). The mixture was stirred at 25° C. for 12 h. LCMS showed starting material was consumed and the desired mass was detected. The reaction was concentrated under reduced pressure to give compound 1-[2-[2-(6-methoxycarbonyl-2-pyridyl)-1,3,3a, 4,6,6a-hexahydropyrrolo[3,4-c]pyrrole5-carbonyl]-8-methyl-[1,2,4]triazolo[1,5-a]pyridin-6-yl]-3-methyl-pyrrolidine-3-carboxylic acid (500 mg, crude) as a yellow oil.

LC-MS [ESI, M+1]: 534.3.

### Step 5: methyl 6-[5-[6-[3-(acetamidocarbamoyl)-3-methyl-pyrrolidin-1-yl]-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate

To a solution of 1-[2-[2-(6-methoxycarbonyl-2-pyridyl)-1,3,3a, 4,6,6a-hexahydropyrrolo[3,4-c]pyrrole-5-carbonyl]-8-methyl-[1,2,4]triazolo[1,5-a]pyridin-6-yl]-3-methyl-pyrrolidine-3-carboxylic acid (500 mg, 937.07 μmol, 1 eq) and acetohydrazide (104.13 mg, 1.41 mmol, 1.5 eq) in DMF (4 mL) was added HATU (1.07 g, 2.81 mmol, 3 eq) and DIEA (605.55 mg, 4.69 mmol, 816.10 μL, 5 eq). The mixture was stirred at 25° C. for 2 h. LCMS showed starting material was consumed and desired mass was detected. The reaction was added water (5 mL) and then extracted with DCM (10 mL×3). The combined organic phase was washed with brine (10 mL), dried over anhydrous Na2SO4, filtered and concentrated in vacuo. The residue was purified by prep-HPLC (column: CD05-Phenomenex luna C18 150*40*10 um; mobile phase: [water(FA)—ACN]; gradient:7%-37% B over 11 min) and lyophilized to give compound methyl 6-[5-[6-[3-(acetamidocarbamoyl)-3-methyl-pyrrolidin-1-yl]-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate (290 mg, 52% yield) as a yellow solid.

LC-MS [ESI, M+1]: 590.3.

### Step 6: methyl 6-[5-[8-methyl-6-[3-methyl-3-(5-methyl-1,3,4-thiadiazol-2-yl)pyrrolidin-1-yl]-[1,2,4]triazolo[1,5-a]pyridine-2-carbothioyl]-1,3,3a, 4,6,6a-hexahydropyrrolo[3,4-c]pyrrol-2-yl]pyridine-2-carboxylate

To a solution of methyl 6-[5-[6-[3-(acetamidocarbamoyl)-3-methyl-pyrrolidin-1-yl]-8-methyl-[1,2,4]triazolo[1,5-a]pyridine-2-carbonyl]-1,3,3a, 4,6,6a-hexahydropyrrolo[3,4-c]pyrrol-2-yl]pyridine-2-carboxylate (100 mg, 169.59 μmol, 1 eq) in toluene (1 mL) was added LAWESSON'S REAGENT (34.30 mg, 84.80 μmol, 0.5 eq). The mixture was stirred at 110° C. for 1 h. LCMS showed starting material was consumed and desired mass was detected. The reaction was concentrated under reduced pressure to give a residue. The residue was purified by prep-HPLC (column: CD01-Phenomenex luna C18 150*25*10 um; mobile phase: [water(FA)—ACN]; gradient:27%-57% B over 10 min) and lyophilized to give compound methyl 6-[5-[8-methyl-6-[3-methyl-3-(5-methyl-1,3,4-thiadiazol-2-yl)pyrrolidin-1-yl]-[1,2,4]triazolo[1,5-a]pyridine-2-carbothioyl]-1,3,3a, 4,6,6a-hexahydropyrrolo[3,4-c]pyrrol-2-yl]pyridine-2-carboxylate (9.1 mg, 9% yield) as a yellow solid.

LC-MS [ESI, M+1]: 604.2

| | | | |
|---|---|---|---|
| Cmpd. No. | Synthesis | LC/MS | ¹H NMR |

| | | |
|---|---|---|
| 430 | Similar to Example | m/z 501.3 [M + H]⁺ |
| | 38 | |
| 431 | Similar to Example | m/z 550.2 [M + H]⁺ |
| | 38 | |
| 432 | Similar to Example | m/z 568.2 [M + H]⁺ |
| | 38 | |

### Example 39

### (R)-N-(2-hydroxy-2-methylpropyl)-4-methyl-6-(3-methyl-3-phenylpyrrolidin-1-yl)pyrazolo[1,5-a]pyridine-2-carboxamide (Compound 39)

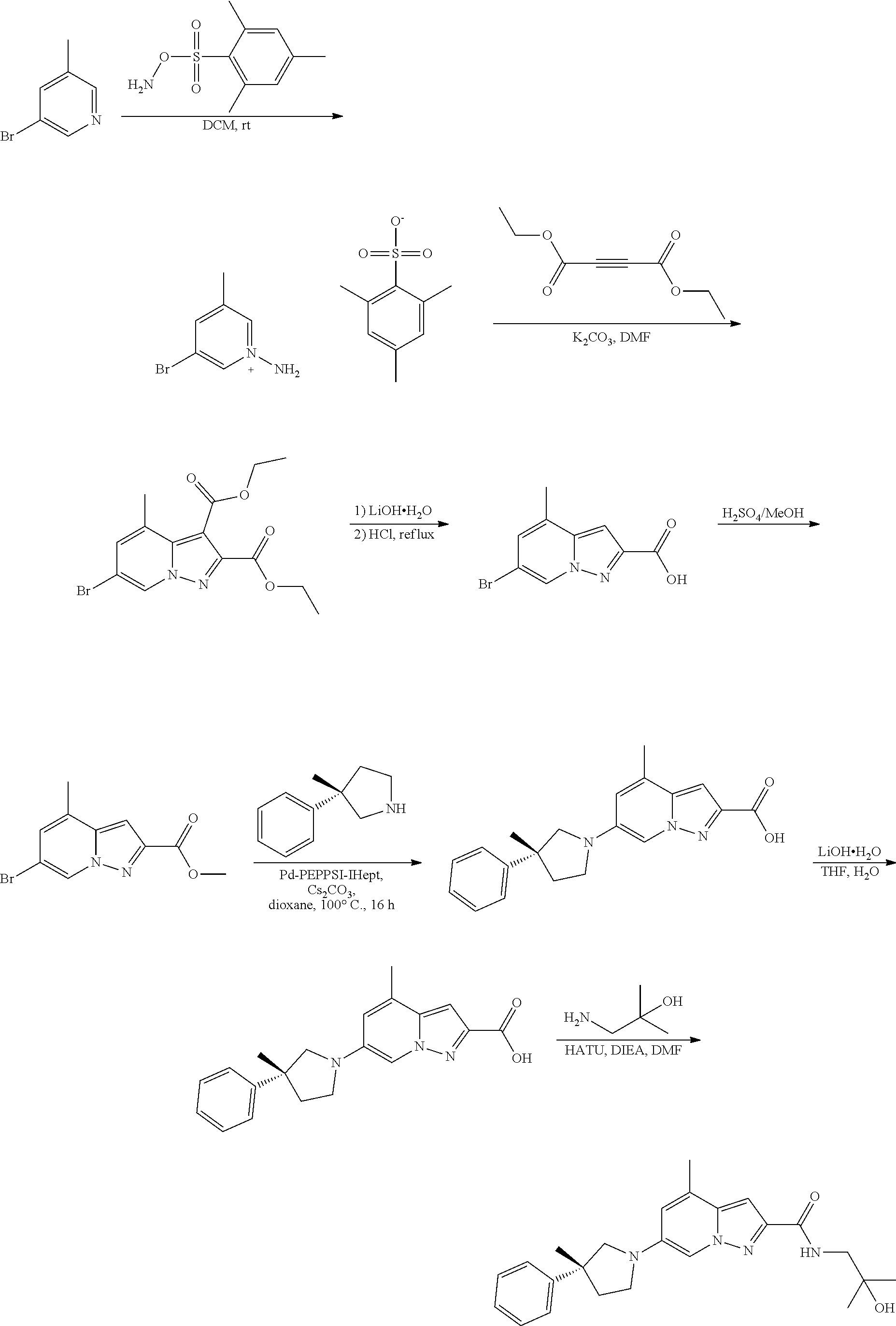

### Step 1: 3-bromo-5-methyl-pyridin-1-ium-1-amine;2,4,6-trimethylbenzenesulfonate

To a solution of 3-bromo-5-methyl-pyridine (5 g, 29.07 mmol, 1 eq) in DCM (10 mL) was added amino 2,4,6-trimethylbenzenesulfonate (7.51 g, 34.88 mmol, 1.2 eq) in DCM (20 mL). The mixture was stirred at 25° C. for 1 h. LCMS showed starting material was consumed completely and the desired compound was detected. The residue was concentrated, then triturated with DCM (200 mL) for 20 min to give 3-bromo-5-methyl-pyridin-1-ium-1-amine;2,4,6-trimethylbenzenesulfonate (10.2 g, 91% yield) as white solid.

LC-MS [ESI, M+1]: 186.9.

¹H NMR (400 MHz, DMSO-d₆) δ=8.95 (s, 1H), 8.68 (s, 1H), 8.54-8.42 (m, 2H), 6.75 (s, 2H), 2.51 (d, J=1.6 Hz, 6H), 2.44 (s, 3H), 2.18 (s, 3H).

### Step 2: diethyl 6-bromo-4-methyl-pyrazolo[1,5-a]pyridine-2,3-dicarboxylate

Diethyl but-2-ynedioate (878.73 mg, 5.16 mmol, 826.65 μL, 2 eq) was added to a solution of 3-bromo-5-methylpyridin-1-ium-1-amine;2,4,6-trimethylbenzenesulfonate (1 g, 2.58 mmol, 1 eq) and K₂CO₃ (713.70 mg, 5.16 mmol, 2 eq) in DMF (10 mL). The reaction mixture was stirred at 80° C. for 1 h. LCMS showed starting material was consumed completely and the desired compound was detected. The mixture was quenched with water (20 mL) and extracted with EtOAc (10 mL×3), the combined organic phase was dried over anhydrous sodium sulfate, filtered and concentrated to give a residue. The residue was purified by flash silica gel chromatography (ISCO®; 40 g Sepa Flash® Silica Flash Column, Eluent of 0~30% PE/EtOAc gradient) to give diethyl 6-bromo-4-methyl-pyrazolo[1,5-a]pyridine-2,3-dicarboxylate (320 mg, 38% yield) as yellow solid.

LC-MS [ESI, M+23]: 376.9.

¹H NMR (400 MHz, CHLOROFORM-d) δ=8.53 (s, 1H), 7.17 (s, 1H), 4.51-4.38 (m, 4H), 2.57 (s, 3H), 1.42 (td, J=7.2, 12.8 Hz, 6H).

### Step 3: 6-bromo-4-methyl-pyrazolo[1,5-a]pyridine-2-carboxylic acid

A mixture of diethyl 6-bromo-4-methyl-pyrazolo[1,5-a]pyridine-2,3-dicarboxylate (1.5 g, 4.22 mmol, 1 eq), LiOH·H₂O (886.02 mg, 21.12 mmol, 5 eq), THF (10 mL), H₂O (2 mL) was stirred at 70° C. for 12 h. Then HCl/dioxane (4 M, 10.56 mL, 10 eq) was added, the mixture was stirred at 100° C. for 12 h. LCMS showed starting material was consumed completely and the desired mass was detected. The mixture was filtered and concentrated in vacuo. The crude product was triturated with EtOAc:PE (3:1) for 20 min to give 6-bromo-4-methyl-pyrazolo[1,5-a]pyridine-2-carboxylic acid (500 mg, 46% yield) as white solid.

LC-MS [ESI, M+1]: 254.7.

¹H NMR (400 MHz, DMSO-d₆) δ=13.37-12.87 (m, 1H), 9.00 (s, 1H), 7.29 (s, 1H), 7.17 (s, 1H), 2.48 (s, 3H).

### Step 4: methyl 6-bromo-4-methylpyrazolo[1,5-a]pyridine-2-carboxylate

To a solution of 6-bromo-4-methyl-pyrazolo[1,5-a]pyridine-2-carboxylic acid (350 mg, 1.37 mmol, 1 eq) in MeOH (5 mL) was added H2SO4 (13.46 mg, 137.22 μmol, 7.31 μL, 0.1 eq). The mixture was stirred at 70° C. for 1 h. TLC (Rf=0.53, PE:EtOAc=1:1) indicated reactant was consumed completely and one new spot was formed. The mixture was diluted with water (20 mL) and extracted with ethyl acetate (10 mL×3), the combined organic phase was dried over anhydrous sodium sulfate, filtered and concentrated to give a residue. The residue was purified by flash silica gel chromatography (ISCO®; 4 g SepaFlash® Silica Flash Column, Eluent of 0~40% Ethylacetate/Petroleum ether gradient) to give methyl 6-bromo-4-methyl-pyrazolo[1,5-a]pyridine-2-carboxylate (350 mg, 95% yield) as white solid.

¹H NMR (400 MHz, CHLOROFORM-d) δ=8.53 (s, 1H), 7.11 (s, 1H), 7.08 (s, 1H), 4.01 (s, 3H), 2.51 (s, 3H).

### Step 5: methyl 4-methyl-6-[(3R)-3-methyl-3-phenyl-pyrrolidin-1-yl]pyrazolo[1,5-a]pyridine-2-carboxylate

To an 8 mL vial equipped with a magnetic stir bar was added methyl 6-bromo-4-methyl-pyrazolo[1,5-a]pyridine-2-carboxylate (120 mg, 445.94 μmol, 1 eq), (3R)-3-methyl-3-phenyl-pyrrolidine (79.10 mg, 490.53 μmol, 1.1 eq), 1,3-bis[2,6-bis(1-propylbutyl)phenyl]-4,5-dichloro-2H-imidazol-1-ium-2-ide;3-chloropyridine;dichloropalladium (21.69 mg, 22.30 mol, 0.05 eq), Cs₂CO₃ (435.89 mg, 1.34 mmol, 3 eq). The vial was evacuated and backfilled with nitrogen three times. Dioxane (1.5 mL) were added and reaction was stirred at 100° C. for 12 h under N₂ atmosphere. LCMS showed starting material was consumed completely and the desired compound was detected. The mixture was quenched with water (2 mL) and extracted with EtOAc (2 mL×3), the combined organic phase was dried over anhydrous sodium sulfate, filtered and concentrated to give a residue. The residue was purified by flash silica gel chromatography (ISCO®; g SepaFlash® Silica Flash Column, Eluent of 0~50% EtOAc:PE gradient) to give methyl 4-methyl-6-[(3R)-3-methyl-3-phenyl-pyrrolidin-1-yl]pyrazolo[1,5-a]pyridine-2-carboxylate (120 mg, 77% yield) as green oil.

LC-MS [ESI, M+1]: 350.1.

### Step 6: 4-methyl-6-[(3R)-3-methyl-3-phenyl-pyrrolidin-1-yl]pyrazolo[1,5-a]pyridine-2-carboxylic acid

To a solution of methyl 4-methyl-6-[(3R)-3-methyl-3-phenyl-pyrrolidin-1-yl]pyrazolo[1,5-a]pyridine-2-carboxylate (120 mg, 343.42 μmol, 1 eq) in THF (1 mL), H₂O (0.2 mL) was added LiOH·H₂O (43.23 mg, 1.03 mmol, 3 eq). The mixture was stirred at 25° C. for 12 h. LCMS showed starting material was consumed and one main peak with desired mass was detected. The mixture was added 1M HCl to adjust pH=4, then extracted with EtOAc (5 mL×3), the combined organic phase was dried over anhydrous sodium sulfate, filtered and concentrated to give 4-methyl-6-[(3R)-3-methyl-3-phenyl-pyrrolidin-1-yl]pyrazolo[1,5-a]pyridine-2-carboxylic acid (90 mg, 78% yield) as green solid.

### Step 7: N-(2-hydroxy-2-methyl-propyl)-4-methyl-6-[(3R)-3-methyl-3-phenyl-pyrrolidin-1-yl]pyrazolo[1,5-a]pyridine-2-carboxamide

To a solution of 4-methyl-6-[(3R)-3-methyl-3-phenyl-pyrrolidin-1-yl]pyrazolo[1,5-a]pyridine-2-carboxylic acid (20 mg, 59.63 μmol, 1 eq) in DMF (0.5 mL) was added HATU (45.35 mg, 119.26 μmol, 2 eq) and DIEA (23.12 mg, 178.89 μmol, 31.16 μL, 3 eq), 1-amino-2-methyl-propan-2-ol (5.32 mg, 59.63 μmol, 1 eq). The mixture was stirred at 25° C. for 1 h. LCMS showed starting material was consumed and one main peak with desired mass was detected. The reaction was concentrated under reduced pressure to give a residue. The residue was purified by prep-HPLC (column: Welch Xtimate C18 150*25 mm*5 um; mobile phase: [water(FA)—ACN]; gradient:46%-76% B over 10 min and lyophilizated to give compound N-(2-hydroxy-2-methyl-propyl)-4-methyl-6-[(3R)-3-methyl-3-phenyl-pyrrolidin-1-yl]pyrazolo[1,5-a]pyridine-2-carboxamide (18 mg, 75% yield) as a gray solid.

LC-MS [ESI, M+1]: 406.9

¹H NMR (400 MHz, CHLOROFORM-d) δ=7.6 (s, 1H), 7.47 (d, J=5.2 Hz, 1H), 7.40-7.29 (m, 4H), 7.26-7.23 (m, 1H), 7.00 (s, 1H), 6.71 (s, 1H), 3.59 (d, J=8.8 Hz, 1H), 3.56-3.45 (m, 4H), 3.43-3.35 (m, 1H), 2.47 (s, 3H), 2.37-2.32 (m, 1H), 2.25 (dd, J=4.4, 8.4 Hz, 1H), 1.46 (s, 3H), 1.30 (s, 6H).

| Cmpd. No. | Synthesis | LC/MS | ¹H NMR |
|---|---|---|---|
| 433 | Similar to Example 39 | m/z 348.8 | |
| | | [M + H]⁺ | |
| 434 | Similar to Example 39 | m/z 349.1 | ¹H NMR (CHLOROFORM-d, 400 MHz): δ = |
| | | [M + H]⁺ | 8.02 (s, 0.9H), 7.47 (br s, 0.9H), 7.28- |
| | | | 7.41 (m, 4.0H), 7.21-7.26 (m, 1.0H), 7.16 |
| | | | (s, 1.0H), 6.82 (s, 1.0H), 3.31-3.60 (m, |
| | | | 4.0H), 3.03 (br d, J = 5.2 Hz, 3.0H), 2.57 |
| | | | (s, 3.0H), 2.30-2.42 (m, 1.0H), 2.18-2.28 |
| | | | (m, 1.1H), 1.40-1.50 ppm (m, 3.0H) |

### Example 40

### 6-(5-(4-methyl-6-((R)-3-methyl-3-phenylpyrrolidin-1-yl)benzo[d]oxazole-2-carbonyl)hexahydropyrrolo[3,4-c]pyrrol-2(1H)-yl)picolinic acid (Compound 40)

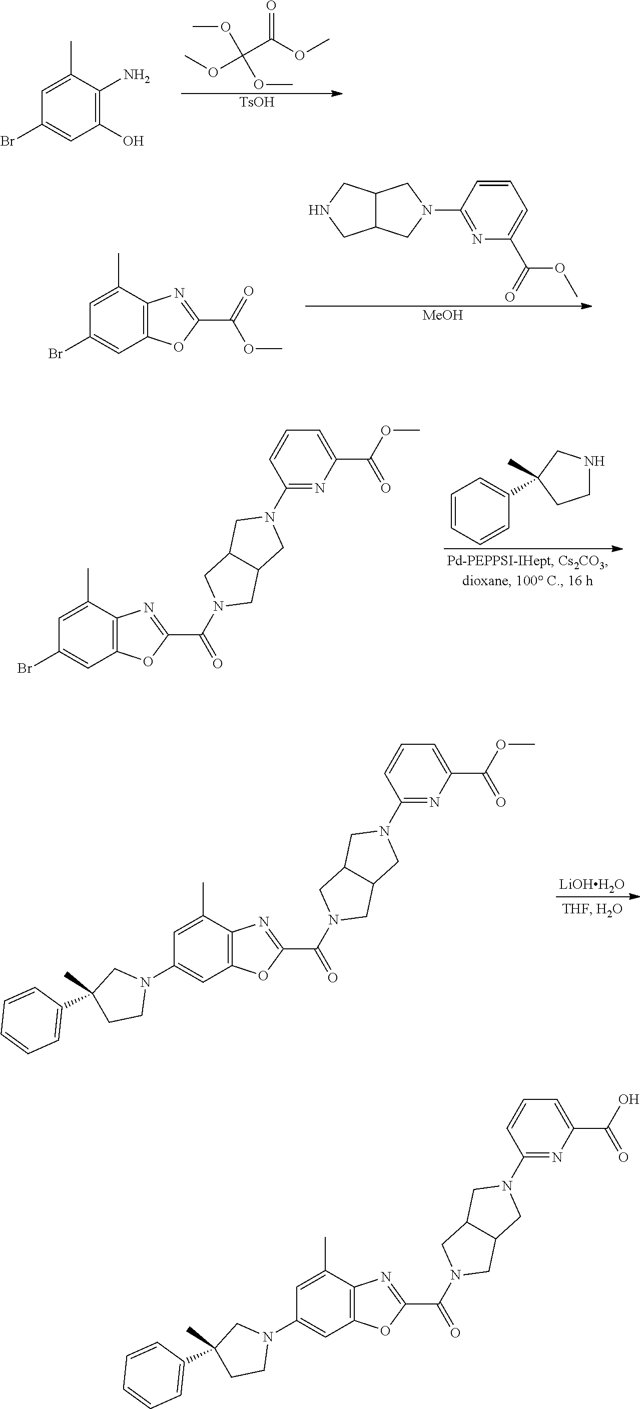

### Step 1: methyl 6-bromo-4-methylbenzo[d]oxazole-2-carboxylate

To a solution of 2-amino-5-bromo-3-methyl-phenol (2 g, 9.90 mmol, 1 eq) in methyl 2,2,2-trimethoxyacetate (16.25 g, 98.99 mmol, 10 eq) was added TsOH (85.23 mg, 494.93 μmol, 0.05 eq). The mixture was stirred at 80° C. for 2 h. LCMS showed starting material was consumed and the desired mass was detected. The reaction mixture was extracted with ethyl acetate, the aqueous phase was extracted with ethyl acetate (30 mL×2). The combined organic phase was washed with brine (50 mL), dried with anhydrous Na2SO4, filtered and concentrated in vacuum, which was purified by column chromatography (PE:EtOAc=5:1, Rf=0.5) to afford methyl 6-bromo-4-methylbenzo[d]oxazole-2-carboxylate (1.8 g, 67%) as a brown solid.

LC-MS [ESI, M+1]: 270.0.

¹H NMR (400 MHz, CHLOROFORM-d) δ=ppm 2.66 (s, 3H) 4.10 (s, 3H) 7.41 (s, 1H) 7.65 (s, 1H).

### Step 2: 6-(5-(6-bromo-4-methylbenzo[d]oxazole-2-carbonyl)hexahydropyrrolo[3,4-c]pyrrol-2(1H)-yl)picolinate

To a solution of methyl 6-bromo-4-methyl-1,3-benzoxazole-2-carboxylate 3 (200 mg, 740.52 μmol, 1 eq) in MeOH (10 mL) was added methyl 6-(2,3,3a, 4,6,6a-hexahydro-1H-pyrrolo[3,4-c]pyrrol-5-yl)pyridine-2-carboxylate (366.25 mg, 1.48 mmol, 2 eq). The mixture was stirred at 50° C. for 12 h. LCMS showed starting material was consumed and the desired mass was detected. The reaction mixture was filtered and the filtrate was concentrated under the reduced pressure to give the product, which was purified by prep-TLC (SiO₂, PE:EtOAc=1:1, Rf=0.40) to afford 6-(5-(6-bromo-4-methylbenzo[d]oxazole-2-carbonyl)hexahydropyrrolo[3,4-c]pyrrol-2(1H)-yl)picolinate (150 mg, 41.3%) as a colourless oil.

LC-MS [ESI, M+1]: 487.0.

¹H NMR (400 MHz, CHLOROFORM-d) δ=ppm 2.61 (s, 3H) 3.08-3.33 (m, 2H) 3.53 (dd, J=10.8, 5.2.00 Hz, 1H) 3.66 (dd, J=10.8, 4.00 Hz, 1H) 3.78-3.91 (m, 3H) 3.95 (s, 3H) 4.06 (dd, J=13.2, 7.6 Hz, 1H) 4.16 (dd, J=12.4, 5.6 Hz, 1H) 4.49 (dd, J=12.4, 7.6 Hz, 1H) 6.57 (d, J=8.4 Hz, 1H) 7.37 (s, 1H) 7.41 (d, J=7.2 Hz, 1H) 7.58 (t, J=7.6 Hz, 1H) 7.63 (s, 1H).

### Step 3: methyl 6-(5-(4-methyl-6-((R)-3-methyl-3-phenylpyrrolidin-1-yl)benzo[d]oxazole-2-carbonyl)hexahydropyrrolo[3,4-c]pyrrol-2(1H)-yl)picolinate

A mixture of methyl 6-[5-(6-bromo-4-methyl-1,3-benzoxazole-2-carbonyl)-1,3,3a, 4,6,6a-hexahydropyrrolo[3,4-c]pyrrol-2-yl]pyridine-2-carboxylate (150 mg, 309.07 μmol, 1 eq), (3R)-3-methyl-3-phenyl-pyrrolidine (64.79 mg, 401.79 μmol, 1.3 eq), 1,3-bis[2,6-bis(1-propylbutyl)phenyl]-4,5-dichloro-2H-imidazol-1-ium-2-ide;3-chloropyridine;dichloropalladium (15.03 mg, 15.45 μmol, 0.05 eq), Cs₂CO₃ (302.10 mg, 927.20 μmol, 3 eq) in dioxane (3 mL) was degassed and purged with N₂ for 3 times, and then the mixture was stirred at 100° C. for 12 h under N₂ atmosphere. LCMS showed starting material was consumed and the desired mass was detected. The reaction mixture was filtered and the filtrate was concentrated under the reduced pressure to give the product. The residue was purified by prep-TLC (SiO₂, PE:EtOAc=1:1, Rf=0.10) to afford methyl 6-(5-(4-methyl-6-((R)-3-methyl-3-phenylpyrrolidin-1-yl)benzo[d]oxazole-2-carbonyl)hexahydropyrrolo[3,4-c]pyrrol-2(1H)-yl)picolinate (180 mg, 99%) as a brown oil.

LC-MS [ESI, M+1]: 566.3.

¹H NMR (400 MHz, CHLOROFORM-d) δ=ppm 1.45 (s, 3H) 2.19-2.28 (m, 1H) 2.30-2.41 (m, 1H) 2.57 (s, 3H) 3.07-3.27 (m, 2H) 3.45 (td, J=8.8, 3.6 Hz, 1H) 3.49-3.60 (m, 3H) 3.61-3.69 (m, 2H) 3.76-3.89 (m, 3H) 3.94 (s, 3H) 4.05 (dd, J=13.2, 7.2 Hz, 1H) 4.18 (dd, J=12.4, 5.2 Hz, 1H) 4.51 (dd, J=12.4, 7.6 Hz, 1H) 6.48-6.60 (m, 3H) 7.22-7.26 (m, 1H) 7.30-7.35 (m, 3H) 7.36-7.43 (m, 2H) 7.57 (t, J=7.6 Hz, 1H).

### Step 4: 6-(5-(4-methyl-6-((R)-3-methyl-3-phenylpyrrolidin-1-yl)benzo[d]oxazole-2-carbonyl)hexahydropyrrolo[3,4-c]pyrrol-2(1H)-yl)picolinic acid

To a solution of methyl 6-[5-[4-methyl-6-[rac-(3R)-3-methyl-3-phenyl-pyrrolidin-1-yl]-1,3-benzoxazole-2-carbonyl]-1,3,3a, 4,6,6a-hexahydropyrrolo[3,4-c]pyrrol-2-yl]pyridine-2-carboxylate (100 mg, 176.78 μmol, 1 eq) in THF (3 mL) and H₂O (1 mL) was added LiOH·H₂O (22.26 mg, 530.35 μmol, 3 eq). The mixture was stirred at 25° C. for 12 h. LCMS showed starting material was consumed and the desired mass was detected. Acidify the clear solution with FA. The reaction mixture was concentrated under the reduced pressure to give the product. The residue was purified by prep-HPLC (column: CD01-Phenomenex luna C18 150*25*10 um; mobile phase: [water(FA)—ACN]; gradient:46%-76% B over 10 min) and lyophilized to give 6-(5-(4-methyl-6-((R)-3-methyl-3-phenylpyrrolidin-1-yl)benzo[d]oxazole-2-carbonyl)hexahydropyrrolo[3,4-c]pyrrol-2(1H)-yl)picolinic acid (23.96 mg, 25%) as a yellow solid.

LC-MS [ESI, M+1]: 522.3.

¹H NMR (400 MHz, CHLOROFORM-d) δ=ppm 1.45 (s, 3H) 2.20-2.28 (m, 1H) 2.31-2.40 (m, 1H) 2.57 (s, 3H) 3.12-3.22 (m, 1H) 3.22-3.31 (m, 1H) 3.41-3.68 (m, 6H) 3.77-3.91 (m, 3H) 4.09 (br dd, J=13.2, 7.6 Hz, 1H) 4.22 (br dd, J=12.4, 5.6 Hz, 1H) 4.55 (br dd, J=12.8, 7.6 Hz, 1H) 6.52 (br s, 2H) 6.65 (d, J=8.8 Hz, 1H) 7.23-7.27 (m, 1H) 7.29-7.40 (m, 4H) 7.51 (d, J=7.2 Hz, 1H) 7.68 (t, J=7.6 Hz, 1H).

| Cmpd. No. | Synthesis | LC/MS | ¹H NMR |
|---|---|---|---|
| 435 | Similar to | m/z 350.2 | |
| | Example 40 | M+H]+ | |

### Example 41

### (R)-2-methyl-2-(1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxamido)propanoic acid (Compound 440)

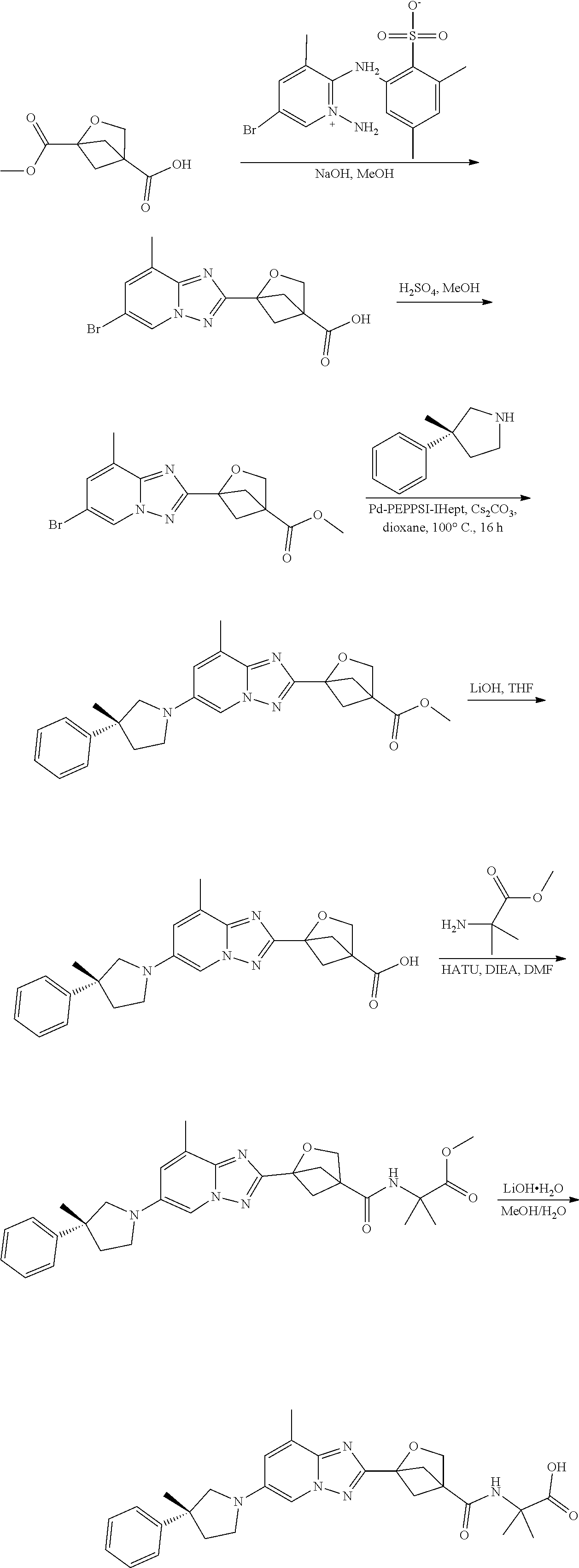

### Step 1: 1-(6-bromo-8-methyl-[1,2,4]triazolo[1,5-a]pyridin-2-yl)-2-oxabicyclo[2.1.1]hexane-4-carboxylic acid

To a solution of 1-methoxycarbonyl-2-oxabicyclo[2.1.1]hexane-4-carboxylic acid (300 mg, 1.61 mmol, 1 eq) in MeOH (3 mL) was added NaOH (96.68 mg, 2.42 mmol, 1.5 eq) and 5-bromo-3-methyl-pyridin-1-ium-1,2-diamine;2,4,6-trimethylbenzenesulfonate (777.98 mg, 1.93 mmol, 1.2 eq) The mixture was stirred at 25° C. for 12 h. LCMS showed starting material was consumed and the desired mass was detected. The reaction mixture was filtered and the filtrate was concentrated under the reduced pressure to give compound 1-(6-bromo-8-methyl-[1,2,4]triazolo[1,5-a]pyridin-2-yl)-2-oxabicyclo[2.1.1]hexane-4-carboxylic acid (550 mg, crude) as yellow solid.

LC-MS [ESI, M+1]:339.9.

### Step 2: methyl 1-(6-bromo-8-methyl-[1,2,4]triazolo[1,5-a]pyridin-2-yl)-2-oxabicyclo[2.1.1]hexane-4-carboxylate

To a solution of 1-(6-bromo-8-methyl-[1,2,4]triazolo[1,5-a]pyridin-2-yl)-2-oxabicyclo[2.1.1]hexane-4-carboxylic acid (550 mg, 1.63 mmol, 1 eq) in MeOH (6 mL) was added H2SO4 (159.52 mg, 1.63 mmol, 86.70 μL, 1 eq). The mixture was stirred at 70° C. for 1 h. LCMS showed starting material was consumed and the desired mass was detected. The pH was adjusted to around 7 by aqeuous. NaHCO₃, and then extracted with ethyl acetate (20 mL×2). The combined organic layers were washed with brine (40 mL), dried over Na2SO4, filtered and concentrated under reduced pressure to give a residue. The residue was purified by column chromatography (SiO2, PE:EtOAc=1:1, Rf=0.50) to give compound methyl 1-(6-bromo-8-methyl-[1,2,4]triazolo[1,5-a]pyridin-2-yl)-2-oxabicyclo[2.1.1]hexane-4-carboxylate (160 mg, 454.31 μmol, 27.93% yield) as white solid.

LC-MS [ESI, M+1]:353.8.

¹H NMR (400 MHz, CHLOROFORM-d) δ=8.55 (s, 1H), 7.40 (s, 1H), 4.18 (s, 2H), 3.80 (s, 3H), 2.70 (d, J=5.6 Hz, 2H), 2.66 (s, 3H), 2.35 (d, J=5.6 Hz, 2H).

### Step 3: methyl (R)-1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxylate

A mixture of methyl 1-(6-bromo-8-methyl-[1,2,4]triazolo[1,5-a]pyridin-2-yl)-2-oxabicyclo[2.1.1]hexane-4-carboxylate (160 mg, 454.31 μmol, 1 eq), (3R)-3-methyl-3-phenyl-pyrrolidine (80.58 mg, 499.74 μmol, 1.1 eq), 1,3-bis[2,6-bis(1-propylbutyl)phenyl]-4,5-dichloro-2H-imidazol-1-ium-2-ide;3-chloropyridine;dichloropalladium (22.10 mg, 22.72 μmol, 0.05 eq) and Cs2CO3 (370.06 mg, 1.14 mmol, 2.5 eq) in dioxane (2 mL) was degassed and purged with N₂ for 3 times, and then the mixture was stirred at 100° C. for 12 h under N₂ atmosphere. LCMS showed starting material was consumed and the desired mass was detected. The reaction mixture was filtered and the filtrate was concentrated under the reduced pressure to give the product. The residue was purified by prep-TLC (SiO₂, PE:EtOAc=1:3, Rf=0.5) to give compound methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (185 mg, 413.86 μmol, 91.10% yield, 96.757% purity) as gray gum.

LC-MS [ESI, M+1]:433.3.

¹H NMR (400 MHz, CHLOROFORM-d) δ=7.63 (s, 1H), 7.39-7.29 (m, 4H), 7.26-7.22 (m, 1H), 6.94 (s, 1H), 4.16 (s, 2H), 3.79 (s, 3H), 3.59 (d, J=8.8 Hz, 1H), 3.55-3.43 (m, 2H), 3.38 (dt, J=3.4, 8.7 Hz, 1H), 2.68 (br d, J=4.4 Hz, 2H), 2.63 (s, 3H), 2.41-2.32 (m, 3H), 2.30-2.20 (m, 1H), 1.46 (s, 3H).

### Step 4: (R)-1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxylic acid

To a solution of methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (100 mg, 231.21 μmol, 1 eq) in MeOH (1 mL) and H₂O (1 mL) was added LiOH·H₂O (19.40 mg, 462.41 μmol, 2 eq). The mixture was stirred at 25° C. for 1 h. LCMS showed starting material was consumed and the desired mass was detected. The reaction mixture was concentrated under the reduced pressure to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (96 mg, crude) as yellow oil.

LC-MS [ESI, M+1]:419.2.

### Step 5: methyl (R)-2-methyl-2-(1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxamido)propanoate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (30 mg, 71.69 μmol, 1 eq) in DMF (1 mL) was added methyl 2-amino-2-methyl-propanoate (10.08 mg, 86.02 μmol, 1.2 eq), HATU (32.71 mg, 86.02 μmol, 1.2 eq) and DIEA (27.80 mg, 215.06 μmol, 37.46 μL, 3 eq). The mixture was stirred at 25° C. for 1 h. LCMS showed starting material was consumed and the desired mass was detected. The mixture was quenched with water (2 mL) and extracted with ethyl acetate (3 mL×2). The combined organic phase was washed with brine (2 mL), dried with anhydrous Na2SO4, filtered and concentrated in vacuum. The residue was purified by prep-HPLC (FA condition column: Phenomenex luna C18 150*25 mm*10 um; mobile phase: [water(FA)—ACN]; gradient:30%-60% B over 10 min) to give compound methyl 2-methyl-2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carbonyl]amino]propanoate (30 mg, 57.96 μmol, 80.85% yield) as yellow oil.

LC-MS [ESI, M+1]:518.3.

### Step 6: (R)-2-methyl-2-(1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxamido)propanoic acid

To a solution of methyl 2-methyl-2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carbonyl]amino]propanoate (30 mg, 57.96 μmol, 1 eq) in THF (1 mL) and H₂O (0.3 mL) was added LiOH·H₂O (4.86 mg, 115.92 μmol, 2 eq). The mixture was stirred at 25° C. for 0.5 h. LCMS showed starting material was consumed and the desired mass was detected. The reaction mixture was concentrated under the reduced pressure to give the crude product. The residue was purified by prep-HPLC (FA condition column: Phenomenex luna C18 150*25 mm*10 um; mobile phase: [water(FA)—ACN]; gradient:35%-65% B over 10 min) to give compound 2-methyl-2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carbonyl]amino]propanoic acid (28 mg, 54.35 μmol, 93.77% yield, 97.742% purity) as white solid.

LC-MS [ESI, M+1]:504.3.

¹H NMR (400 MHz, CHLOROFORM-d) δ=7.65 (s, 1H), 7.40-7.34 (m, 2H), 7.33-7.29 (m, 2H), 7.27-7.21 (m, 2H), 7.01 (s, 1H), 4.21 (s, 2H), 3.60 (d, J=8.8 Hz, 1H), 3.55-3.44 (m, 2H), 3.39 (dt, J=3.6, 8.8 Hz, 1H), 2.96-2.89 (m, 2H), 2.60 (s, 3H), 2.39 (td, J=8.4, 12.2 Hz, 1H), 2.31 (br d, J=4.0 Hz, 2H), 2.29-2.23 (m, 1H), 1.76 (s, 6H), 1.47 (s, 3H).

| Compound | | |
|---|---|---|
| Number | Synthesis | LC/MS |
| 441 | Similar to | m/z 504.3 [M + H]⁺ |
| | Example 41 | |
| 442 | Similar to | m/z 476.1 [M + H]⁺ |
| | Example 41 | |
| 443 | Similar to | m/z 460.2 [M + H]⁺ |
| | Example 41 | |
| 444 | Similar to | m/z 476.1 [M + H]⁺ |
| | Example 41 | |
| 445 | Similar to | m/z 528.1 [M + H]⁺ |
| | Example 41 | |
| 446 | Similar to | m/z 488.2 [M + H]⁺ |
| | Example 41 | |
| 447 | Similar to | m/z 470.2 [M + H]⁺ |
| | Example 41 | |
| 448 | Similar to | m/z 513.2 [M + H]⁺ |
| | Example 41 | |
| 449 | Similar to | m/z 470.2 [M + H]⁺ |
| | Example 41 | |
| 450 | Similar to | m/z 470.3 [M + H]⁺ |
| | Example 41 | |
| 451 | Similar to | m/z 470.2 [M + H]⁺ |
| | Example 41 | |
| 452 | Similar to | m/z 553.2 [M + H]⁺ |
| | Example 41 | |
| 453 | Similar to | m/z 553.2 [M+H]⁺ |
| | Example 41 | |
| 454 | Similar to | m/z 554.2 [M+H]⁺ |
| | Example 41 | |
| 455 | Similar to | m/z 539.2 [M+H]⁺ |
| | Example 41 | |
| 456 | Similar to | m/z 539.2 [M+H]⁺ |
| | Example 41 | |
| 458 | Similar to | m/z 504.3 [M+H]⁺ |
| | Example 41 | |

### Example 42

### (R)-3-hydroxy-3-methyl-N-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methyl)butanamide (Compound 457)

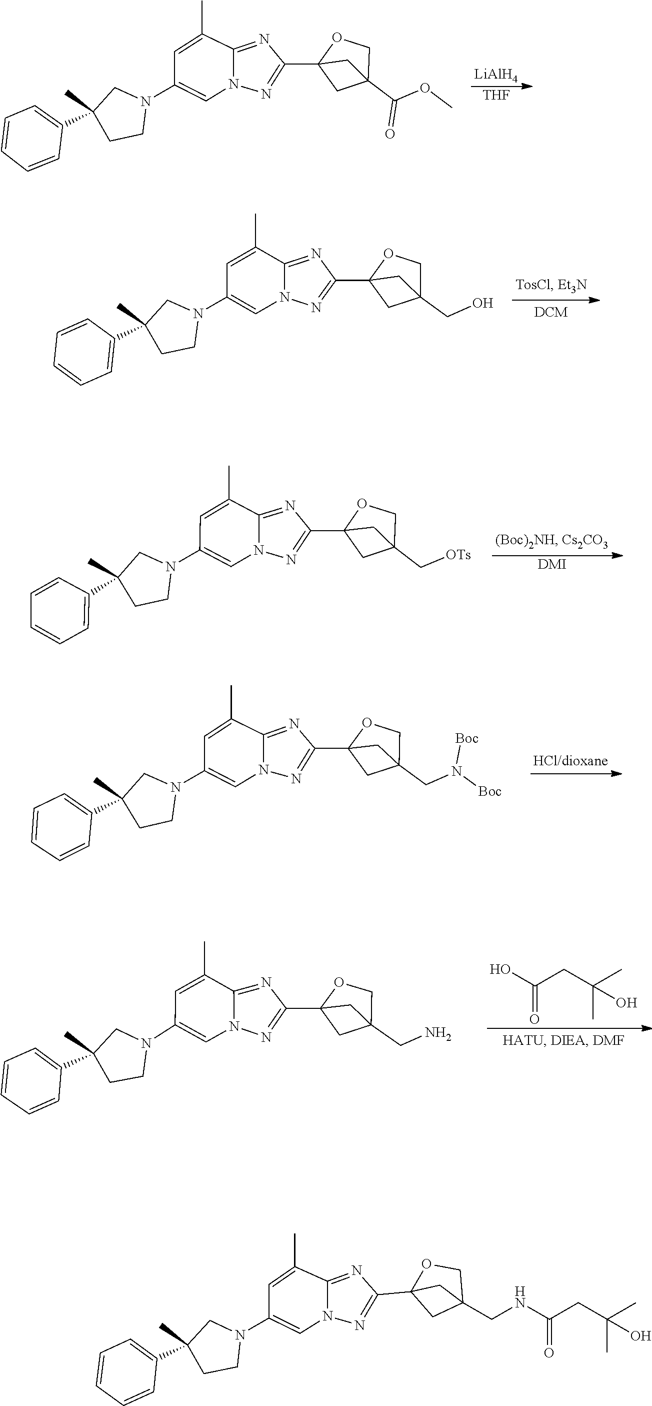

### Step 1: (R)-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanol

To a solution of LiAlH₄ (2.5 M, 363.21 μL, 2 eq) in THF (3 mL) was added methyl 1-[6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (190 mg, 454.02 μmol, 1 eq) in five portions at 0° C. under N₂. The reaction mixture was stirred at 25° C. for 1 h under N₂. LCMS showed starting material was consumed and one main peak with desired mass was detected. After the reaction mixture was cooled to 0° C., the reaction mixture was quenched by addition of (0.5 mL) of H₂O, followed by 0.5 mL of 15% aqueous NaOH and 1.5 mL of H₂O.and then extracted with ethyl acetate (10 mL×3).The combined organic phase was washed with brine (5 mL), dried with anhydrous Na2SO4, filtered and concentrated in vacuum to give compound (R)-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanol (130 mg, 321.38 μmol, 70.79% yield) as a yellow gum.

LC-MS [ESI, M+1]: 405.1.

### Step 2: [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl 4-methylbenzenesulfonate

To a solution of (R)-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanol (130 mg, 321.38 μmol, 1 eq), DMAP (3.93 mg, 32.14 μmol, 0.1 eq) and TEA (97.56 mg, 964.14 μmol, 134.20 L, 3 eq) in DCM (3 mL) was added TosCl (122.54 mg, 642.76 μmol, 2 eq) at 0° C. The mixture was stirred at 25° C. for 1 h. LCMS showed starting material was consumed and the desired mass was detected. The reaction mixture was concentrated under the reduced pressure to give the product. The reaction was added water (5 mL) and then extracted with ethyl acetate (5 mL×3). The combined organic phase was washed with brine (5 mL), dried with anhydrous Na2SO4, filtered and concentrated in vacuum. The residue was purified by prep-TLC (SiO2, PE:EtOAc=0:1,Rf=0.5) to give compound [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl 4-methylbenzenesulfonate (105 mg, 58% yield) as a green gum.

LC-MS [ESI, M+1]: 559.2.

¹H NMR (400 MHz, CHLOROFORM-d) δ=7.83 (d, J=8.0 Hz, 2H), 7.61 (s, 1H), 7.37 (t, J=8.0 Hz, 4H), 7.33-7.28 (m, 2H), 7.26-7.22 (m, 1H), 6.93 (s, 1H), 4.39 (s, 2H), 3.87 (s, 2H), 3.58 (d, J=8.8 Hz, 1H), 3.54-3.44 (m, 2H), 3.37 (dt, J=3.6, 8.8 Hz, 1H), 2.62 (s, 3H), 2.47 (s, 3H), 2.39-2.33 (m, 1H), 2.29-2.20 (m, 3H), 2.09-2.05 (m, 2H), 1.46 (s, 3H)

### Step 3: tert-butyl (R)-(tert-butoxycarbonyl)((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methyl)carbamate

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl 4-methylbenzenesulfonate (105 mg, 187.94 μmol, 1 eq) in DMI (1 mL) was added Cs2CO3 (183.70 mg, 563.82 μmol, 3 eq) and tert-butyl N-tert-butoxycarbonylcarbamate (81.66 mg, 375.88 μmol, 2 eq). The mixture was stirred at 75° C. for 1 h. LCMS showed starting material was consumed and the desired mass was detected. The reaction was added water (5 mL) and then extracted with ethyl acetate (10 mL×3). The combined organic phase was washed with brine (5 mL), dried with anhydrous Na2SO4, filtered and concentrated in vacuum. The residue was purified by prep-TLC (SiO₂, PE:EtOAc=0:1, Rf=0.7) to give compound tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate (75 mg, 66% yield) as a yellow gum.

LC-MS [ESI, M+1]: 604.3

### Step 4: (R)-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanamine

To a solution of tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate (75 mg, 124.22 μmol, 1 eq) in DCM (1 mL) was added TFA (767.50 mg, 6.73 mmol, 0.5 mL, 54.19 eq). The mixture was stirred at 25° C. for 1 h. LCMS showed starting material was consumed and the desired mass was detected. The reaction mixture was concentrated under reduced pressure to give a residue to give compound [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanamine (58 mg, 90% yield, TFA) as a red gum.

LC-MS [ESI, M+1]: 404.1.

### Step 5: (R)-3-hydroxy-3-methyl-N-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methyl)butanamide

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanamine (20 mg, 38.64 μmol, 1 eq, TFA) and 3-hydroxy-3-methyl-butanoic acid (4.57 mg, 38.64 μmol, 1 eq) in DMF (0.5 mL) was added HATU (29.39 mg, 77.29 u mol, 2 eq). The mixture was stirred at 25° C. for 0.5 h, and then DIEA (24.97 mg, 193.22 u mol, 33.66 u L, 5 eq) was added at 25° C. The mixture was stirred at 25° C. for 1 h. LCMS showed starting material was consumed and one main peak with desired mass was detected. The reaction mixture was filtered. The residue was purified by prep-HPLC (NH4HCO3 condition column: Waters xbridge 150*25 mm 10 um; mobile phase: [water(NH₄HCO₃)—ACN]; gradient:28%-48% B over 10 min) to give compound 3-hydroxy-3-methyl-N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]butanamide (1.55 mg, 7% yield) as a yellow solid.

LC-MS [ESI, M+1]: 504.2

¹H NMR (400 MHz, CHLOROFORM-d) δ=7.63 (s, 1H), 7.40-7.34 (m, 2H), 7.33-7.29 (m, 2H), 7.27-7.23 (m, 1H), 6.94 (s, 1H), 6.17 (br s, 1H), 3.91 (s, 3H), 3.74 (br d, J=6.0 Hz, 2H), 3.59 (br d, J=9.2 Hz, 1H), 3.52-3.45 (m, 2H), 3.41-3.34 (m, 1H), 2.63 (s, 3H), 2.39 (s, 3H), 2.24 (br s, 3H), 2.10 (br d, J=4.4 Hz, 2H), 1.46 (s, 3H), 1.31 (s, 6H).

| Compound # | Synthesis | LC/MS |
|---|---|---|
| 458 | Similar to | m/z |
| | Example 42 | 418.0 [M + H]⁺ |

### Example 43

### 2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methoxy]acetic acid (Compound 459)

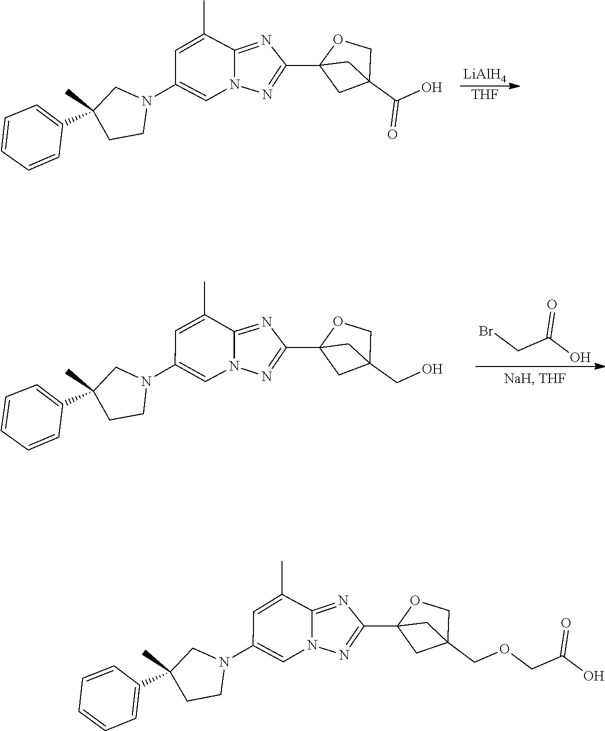

### Step 1: [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanol

To a solution of LiAlH₄ (2.5 M, 1.15 mL, 4 eq) in THF (10 mL) was added 1-[8-methyl-6-[(3R)-3-methyl-3-phenyl-pyrrolidin-1-yl]-[1,2,4]triazolo[1,5-a]pyridin-2-yl]-2-oxabicyclo [2.1.1]hexane-4-carboxylic acid (300 mg, 716.87 μmol, 1 eq) in THF (5 mL) at 0° C. The mixture was stirred at 25° C. for 4 h. LCMS showed starting material was consumed and one main peak with desired mass was detected. The residue was purified by column chromatography (SiO2, PE:EtOAc=0:1, Rf=0.34) to give [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanol (260 mg, 578.49 μmol, 80.70% yield, 90% purity) as colorless oil.

LC-MS [ESI, M+1]: 405.2

¹H NMR (400 MHz, CHLOROFORM-d) δ=7.43 (s, 1H), 7.19-7.13 (m, 2H), 7.12-7.08 (m, 2H), 7.05-7.00 (m, 1H), 6.72 (s, 1H), 3.85-3.70 (m, 4H), 3.38 (d, J=8.8 Hz, 1H), 3.31-3.23 (m, 2H), 3.17 (dt, J=3.6, 8.8 Hz, 1H), 2.43 (s, 3H), 2.18-2.08 (m, 3H), 2.06-2.00 (m, 1H), 1.90 (br d, J=4.8 Hz, 2H), 1.25 (s, 3H)

### Step 2: 2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methoxy]acetic acid

To a mixture of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanol (10 mg, 24.72 μmol, 1 eq) in THF (2 mL) and carefully added NaH (1.48 mg, 37.08 μmol, 60% purity, 1.5 eq) at 0° C., the mixture was stirred at 25° C. for 0.5 h, and then 2-bromoacetic acid (6.87 mg, 49.44 μmol, 3.55 μL, 2 eq) was added to the mixture. The reaction mixture was stirred for 11.5 h at 25° C. LCMS showed starting material was consumed and one main peak with desired mass was detected. The reaction mixture was slowly quenched with H₂O (1 mL), filtered and concentrated under reduced pressure to give a residue. The crude product was purified by prep-HPLC (column: CD04-Welch Utimate C18 150*25*7 um; mobile phase: [water(FA)—ACN]; gradient:24%-54% B over 15 min) to give compound 2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methoxy]acetic acid (4.89 mg, 10.41 μmol, 42.11% yield, 98.46% purity) as yellow solid.

LC-MS [ESI, M+1]: 463.3

¹H NMR (400 MHz, CHLOROFORM-d) δ=7.66-7.59 (m, 1H), 7.32-7.21 (m, 4H), 7.18-7.14 (m, 1H), 6.93-6.83 (m, 1H), 4.12 (br s, 2H), 3.90 (s, 4H), 3.51 (br d, J=8.8 Hz, 1H), 3.46-3.36 (m, 2H), 3.34-3.26 (m, 1H), 2.54 (s, 3H), 2.39-2.24 (m, 3H), 2.21-2.12 (m, 1H), 2.06 (br s, 2H), 1.38 (s, 3H)

### Example 44

### 6-[[2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetyl]amino]pyridine-2-carboxylic acid (Compound 460)

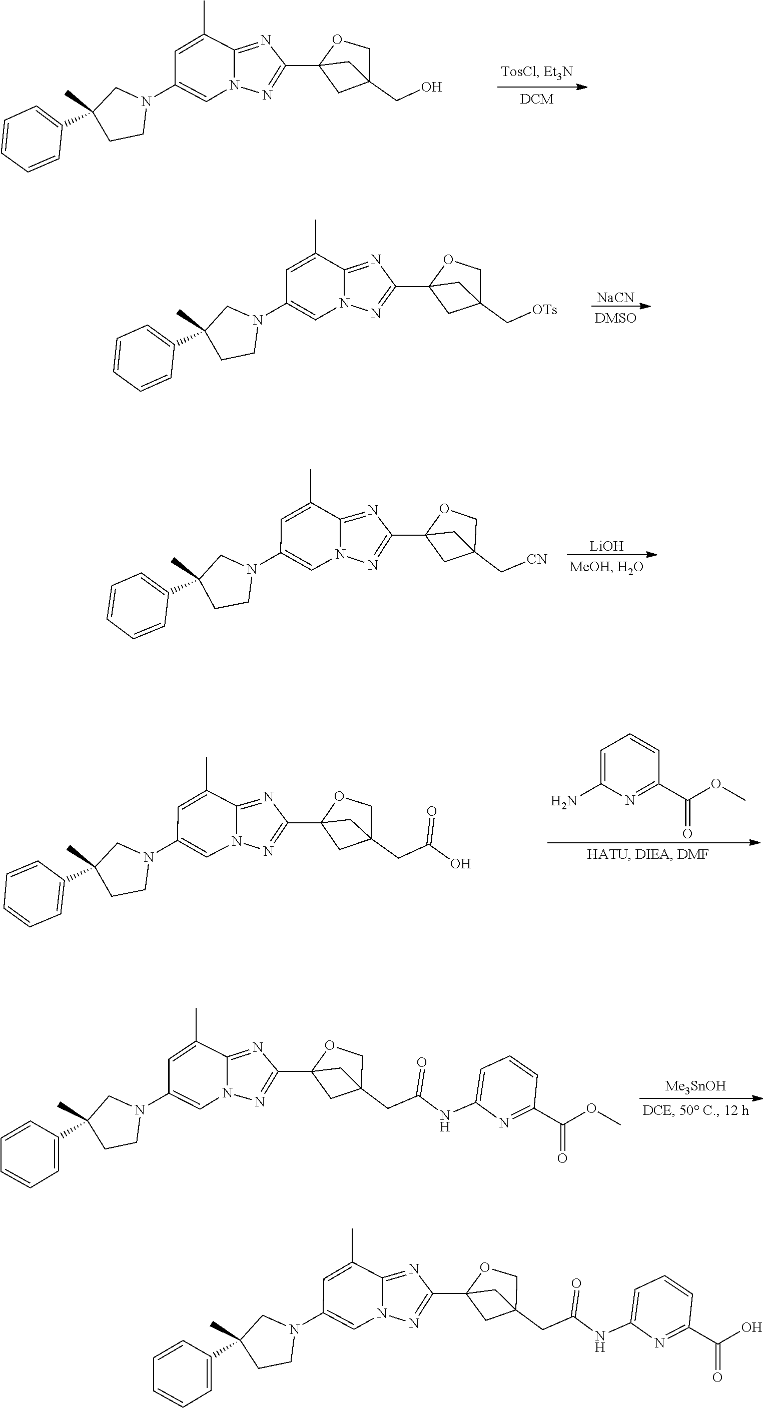

### Step 1: [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl 4-methylbenzenesulfonate

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanol (200 mg, 494.43 μmol, 1 eq) in DCM (4 mL) was added TosCl (188.52 mg, 988.87 μmol, 2 eq) and TEA (100.06 mg, 988.87 μmol, 137.64 μL, 2 eq). The mixture was stirred at 25° C. for 2 h. LCMS showed starting material was consumed and one main peak with desired mass was detected. The mixture was filtered, and concentrated under pressure to give the product. The residue was purified by column chromatography by prep-TLC (SiO2, PE:EtOAc=3:1, Rf=0.3) to give Compound [1-[8-methyl-6-[(3R)-3-methyl-3-phenyl-pyrrolidin-1-yl]-[1,2,4]triazolo[1,5-a]pyridin-2-yl]-2-oxabicyclo [2.1.1]hexan-4-yl]methyl 4-methylbenzenesulfonate (190 mg, 324.78 μmol, 65.69% yield, 95.5% purity) as a white solid.

LC-MS [ESI, M+1]: 559.7

### Step 2: 2-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]acetonitrile

To a solution of [1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]methyl 4-methylbenzenesulfonate (100 mg, 178.99 μmol, 1 eq) in DMSO (6 mL) was added NaCN (26.32 mg, 536.97 μmol, 3 eq). The mixture was stirred at 100° C. for 4 h. LCMS showed starting material was consumed and one main peak with desired mass was detected. The reaction mixture was diluted with water (5 mL) and extracted with EtOAc (5 mL×2). The combined organic layers were washed with water (5 mL), dried over Na2SO4, filtered and concentrated under reduced pressure to give a residue. The residue was purified by column chromatography by prep-TLC (SiO₂, PE:EtOAc=3:1, Rf=0.3) to give compound 2-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]acetonitrile (72 mg, 170.64 μmol, 95.33% yield, 98% purity) as white solid.

LC-MS [ESI, M+1]: 414.2

### Step 3: 2-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]acetic acid

To a solution of 2-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]acetonitrile (72 mg, 174.12 μmol, 1 eq) in MeOH (3 mL) and Water (3 mL) was added LiOH·H₂O (73.07 mg, 1.74 mmol, 10 eq). The mixture was stirred at 100° C. for 4 h. LCMS showed starting material was consumed and one main peak with desired mass was detected. The reaction mixture was diluted with water (5 mL) and extracted with EtOAc (5 mL×3). The combined organic layers were washed with water (5 mL), dried over Na2SO4, filtered and concentrated under reduced pressure to give a residue. The residue was purified by prep-HPLC (column: CD01-Phenomenex luna C18 150*25*10 um; mobile phase: [water (FA)—ACN]; gradient:24%-54% B over 10 min) to give Compound 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetic acid (45 mg, 98.84 μmol, 56.77% yield, 95% purity) as white solid.

LC-MS [ESI, M+1]: 433.3

### Step 4: methyl 6-[[2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetyl]amino]pyridine-2-carboxylate

To a solution of 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetic acid (15 mg, 34.68 μmol, 1 eq) and methyl 6-aminopyridine-2-carboxylate (5.28 mg, 34.68 μmol, 1 eq) in Py (1.5 mL) was added POCl₃ (1 M, 34.68 μL, 1 eq) at 0° C. The mixture was stirred at 25° C. for 1 h. TLC (SiO₂, PE:EtOAc=3:1, Rf=0.3) indicated reactant was consumed completely and some new spots formed. The reaction mixture was diluted with water (2 mL) and extracted with EtOAc (4 mL×2). The combined organic layers were washed with water (5 mL), dried over Na2SO4, filtered and concentrated under reduced pressure to give a residue. The residue was purified by column chromatography by prep-TLC (SiO₂, PE:EtOAc=3:1, Rf=0.3) to give a compound methyl 6-[[2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetyl]amino]pyridine-2-carboxylate (15 mg, 21.18 μmol, 61.06% yield, 80% purity) as black brown solid.

LC-MS [ESI, M+1]: 567.3

### Step 5: 6-[[2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetyl]amino]pyridine-2-carboxylic acid

To a mixture of methyl 6-[[2-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-4-yl]acetyl]amino]pyridine-2-carboxylate (15 mg, 26.47 μmol, 1 eq) in DCE (2 mL) was added hydroxy(trimethyl) stannane (95.73 mg, 529.43 μmol, 20 eq) at 0° C. The mixture was stirred at 60° C. for 1 h. LCMS showed starting material was consumed and the desired mass was detected. The reaction was concentrated under reduced pressure to give a residue. The residue was purified by prep-HPLC (column: CD01-Phenomenex luna C18 150*25*10 um; mobile phase: [water(FA)—ACN]; gradient:34%-64% B over 10 min) to give Compound 6-[[2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetyl]amino]pyridine-2-carboxylic acid (3.84 mg, 6.91 μmol, 26.09% yield, 99.409% purity) as white solid.

LC-MS [ESI, M+1]: 553.3

¹H NMR (400 MHz, METHANOL-d4) δ=8.04-7.84 (m, 3H), 7.72 (br s, 1H), 7.42-7.32 (m, 4H), 7.28-7.16 (m, 2H), 3.94 (s, 2H), 3.64-3.52 (m, 3H), 3.41 (br dd, J=4.8, 8.8 Hz, 1H), 3.02 (br s, 2H), 2.56 (s, 3H), 2.42-2.24 (m, 4H), 2.10 (br s, 2H), 1.44 (s, 3H)

| COMPOUND | | |
|---|---|---|
| # | SYNTHESIS | LC/MS |
| 61 | Similar to | M/Z |
| | example 44 | 525.3 |
| | | [M + H]⁺ |
| 462 | Similar to | M/Z |
| | example 44 | 460.4 |
| | | [M + H]⁺ |
| 463 | Similar to | M/Z |
| | example 44 | 496.2 |
| | | [M + H]⁺ |

### Example 45

### (R)-1-(8-methyl-6-(3-methyl-3-phenylpyrrolidin-1-yl)-[1,2,4]triazolo[1,5-a]pyridin-2-yl)-2-oxabicyclo[2.1.1]hexane-4-carbonitrile (Compound 464)

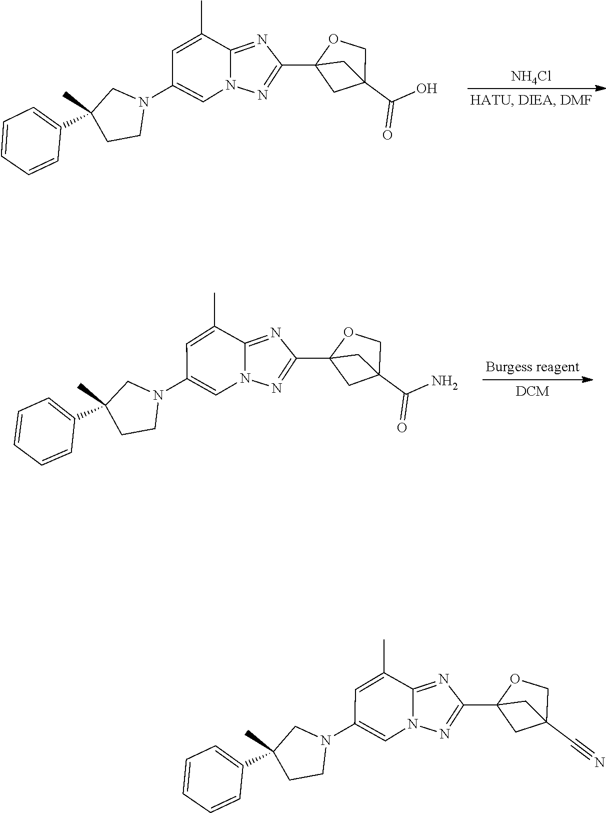 [SMILES: Cc1cc(N2CC[C@](C)(c3ccccc3)C2)cn2nc(C34CC(C(=O)O)(CO3)C4)nc12 ; "NH4Cl" ; Cc1cc(N2CC[C@](C)(c3ccccc3)C2)cn2nc(C34CC(C(N)=O)(CO3)C4)nc12 ; Cc1cc(N2CC[C@](C)(c3ccccc3)C2)cn2nc(C34CC(C#N)(CO3)C4)nc12]

### Step 1: (R)-1-(8-methyl-6-(3-methyl-3-phenylpyrrolidin-1-yl)-[1,2,4]triazolo[1,5-a]pyridin-2-yl)-2-oxabicyclo[2.1.1]hexane-4-carboxamide

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (70 mg, 167.27 μmol, 1 eq) in DMF (2 mL) was added NH₄Cl (89.47 mg, 1.67 mmol, 10 eq), DIEA (64.86 mg, 501.81 μmol, 87.41 μL, 3 eq) and HATU (127.20 mg, 334.54 μmol, 2 eq). The mixture was stirred at 25° C. for 1 h. LCMS showed starting material was consumed and the desired mass was detected. The reaction mixture was filtered and the filtrate was concentrated under the reduced pressure to give the product. The residue was purified by prep-HPLC (FA condition column: Phenomenex luna C18 150*25 mm*10 um; mobile phase: [water(FA)—ACN]; gradient:30%-60% B over 10 min) to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxamide (55 mg, 131.74 μmol, 78.76% yield, 100% purity) as yellow solid.

LC-MS [ESI, M+1]:418.2.

¹H NMR (400 MHz, CHLOROFORM-d) δ=7.63 (s, 1H), 7.40-7.33 (m, 2H), 7.33-7.29 (m, 2H), 7.26 (br s, 1H), 6.95 (s, 1H), 5.80-5.52 (m, 2H), 4.19 (s, 2H), 3.59 (d, J=8.8 Hz, 1H), 3.55-3.44 (m, 2H), 3.38 (dt, J=3.2, 8.8 Hz, 1H), 2.66-2.57 (m, 5H), 2.45-2.32 (m, 3H), 2.31-2.18 (m, 1H), 1.46 (s, 3H).

### Step 2: (R)-1-(8-methyl-6-(3-methyl-3-phenylpyrrolidin-1-yl)-[1,2,4]triazolo[1,5-a]pyridin-2-yl)-2-oxabicyclo[2.1.1]hexane-4-carbonitrile

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxamide (50 mg, 119.76 μmol, 1 eq) in DCM (1 mL) was added methoxycarbonyl-(triethylammonio)sulfonyl-azanide (57.08 mg, 239.52 μmol, 2 eq) at 0-25° C. The mixture was stirred at 0-25° C. for 0.5 h. LCMS showed starting material was consumed and the desired mass was detected. The reaction mixture was concentrated under the reduced pressure to give the crude product. The residue was purified by prep-HPLC (FA condition column: Phenomenex luna C18 150*25 mm*10 um; mobile phase: [water (FA)—ACN]; gradient:46%-76% B over 10 min) to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carbonitrile (38.59 mg, 96.09 μmol, 80.24% yield, 99.474% purity) as white solid.

LC-MS [ESI, M+1]:400.2.

¹H NMR (400 MHz, CHLOROFORM-d) δ=7.62 (s, 1H), 7.40-7.34 (m, 2H), 7.33-7.29 (m, 2H), 7.27-7.23 (m, 1H), 6.96 (s, 1H), 4.20 (s, 2H), 3.59 (d, J=8.8 Hz, 1H), 3.56-3.44 (m, 2H), 3.39 (dt, J=3.6, 8.6 Hz, 1H), 2.79 (br d, J=4.8 Hz, 2H), 2.63 (s, 3H), 2.46 (br d, J=4.8 Hz, 2H), 2.41-2.33 (m, 1H), 2.30-2.21 (m, 1H), 1.47 (s, 3H).

### Example 46

### (R)-6-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)amino)picolinic acid (Compound 465)

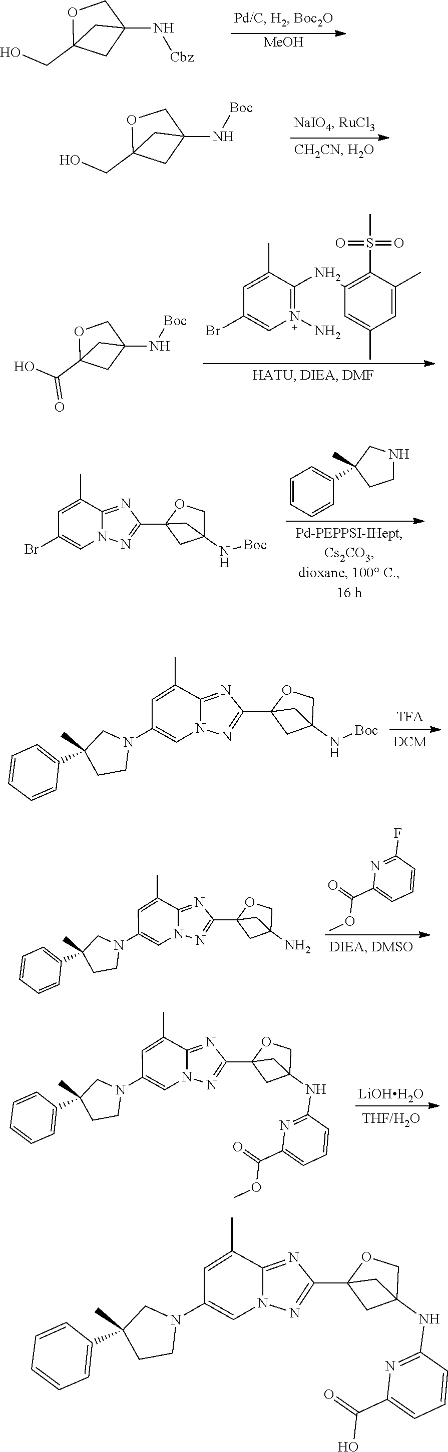

### Step 1: tert-butyl N-[1-(hydroxymethyl)-2-oxabicyclo[2.1.1]hexan-4-yl]carbamate

To a solution of benzyl N-[1-(hydroxymethyl)-2-oxabicyclo[2.1.1]hexan-4-yl]carbamate (10 g, 37.98 mmol, 1 eq) and Boc₂O (24.87 g, 113.94 mmol, 26.18 mL, 3 eq) in EtOH (200 mL) was added Pd/C (2.02 g, 1.90 mmol, 10% purity, 0.05 eq) under H2 atmosphere. The mixture was stirred under H2 (50 Psi) at 25° C. for 12 h. LCMS showed starting material was consumed and one main peak with desired mass was detected. The residue was purified by column chromatography (SiO2, PE:EtOAc=3:1, Rf=0.1) to give compound tert-butyl N-[1-(hydroxymethyl)-2-oxabicyclo[2.1.1]hexan-4-yl]carbamate (7.3 g, 84% yield) as a white solid.

LC-MS [ESI, M+1]:230.2

¹H NMR (400 MHz, CHLOROFORM-d) δ=5.13 (br s, 1H), 3.83 (br d, J=5.6 Hz, 2H), 3.80 (br s, 2H), 2.26-2.05 (m, 1H), 1.95 (br s, 3H), 1.45 (s, 9H).

### Step 2: 4-(tert-butoxycarbonylamino)-2-oxabicyclo[2.1.1]hexane-1-carboxylic acid

To a solution of tert-butyl N-[1-(hydroxymethyl)-2-oxabicyclo[2.1.1]hexan-4-yl]carbamate (3 g, 13.08 mmol, 1 eq), RuCl₃ (81.43 mg, 392.55 μmol, 26.18 μL, 0.03 eq), NaOH (2.09 g, 52.34 mmol, 4 eq) in CH₃CN (18 mL), H₂O (30 mL), and DCM (18 mL) was added NaIO₄ (13.99 g, 65.42 mmol, 3.63 mL, 5 eq) at 0° C. The mixture was stirred at 25° C. for 1 h. LCMS showed starting material was consumed and one main peak with desired mass was detected. The reaction was quenched by water (20 mL) slowly and then the mixture was filtered and washed with water. an aqueous layer was washed with MTBE (10 mL×3). The aqueous layer was acidified with 5M HCl to pH=2 and then extracted with EtOAc (10 mL×3). The combined organic phases were dried over Na2SO4, filtered and concentrated in vacuum. The residue was purified by column chromatography (SiO2, DCM:MeOH=10:1, Rf=0.3) to give compound 4-(tert-butoxycarbonylamino)-2-oxabicyclo [2.1.1]hexane-1-carboxylic acid (1.8 g, 57% yield) as a black solid.

LC-MS [ESI, M+1]:244.2

¹H NMR (400 MHz, DMSO-d₆) δ=7.71 (br s, 1H), 3.66 (br s, 2H), 2.27 (br s, 2H), 1.85 (br d, J=1.6 Hz, 2H), 1.38 (s, 9H).

### Step 3: tert-butyl N-[1-(6-bromo-8-methyl-[1,2,4]triazolo[1,5-a]pyridin-2-yl)-2-oxabicyclo[2.1.1]hexan-4-yl]carbamate

To a solution of 4-(tert-butoxycarbonylamino)-2-oxabicyclo[2.1.1]hexane-1-carboxylic acid (1.5 g, 6.17 mmol, 1 eq) and 5-bromo-3-methyl-pyridin-1-ium-1,2-diamine;2,4,6-trimethylbenzenesulfonate (4.96 g, 12.33 mmol, 2 eq) in DMF (20 mL) was added HATU (3.52 g, 9.25 mmol, 1.5 eq). The mixture was stirred at 25° C. for 0.5 h, and then DIEA (3.98 g, 30.83 mmol, 5.37 mL, 5 eq) was added at 25° C. The mixture was stirred at 25° C. for 1 h. LCMS showed starting material was consumed and one main peak with desired mass was detected. The reaction was added water (10 mL) and then extracted with EtOAc (10 mL×3). The combined organic phase was washed with brine (5 mL), dried with anhydrous Na2SO4, filtered and concentrated in vacuum. The residue was purified by column chromatography (SiO₂, PE:EtOAc=1:1, Rf=0.2) to give compound tert-butyl N-[1-(6-bromo-8-methyl-[1,2,4]triazolo[1,5-a]pyridin-2-yl)-2-oxabicyclo[2.1.1]hexan-4-yl]carbamate (1.8 g, 71% yield) as a brown oil.

LC-MS [ESI, M+1]: 409.0.

### Step 4: tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate

To a solution of tert-butyl N-[1-(6-bromo-8-methyl-[1,2,4]triazolo[1,5-a]pyridin-2-yl)-2-oxabicyclo[2.1.1]hexan-4-yl]carbamate (1 g, 2.44 mmol, 1 eq) and (3R)-3-methyl-3-phenyl-pyrrolidine (393.97 mg, 2.44 mmol, 1 eq) in dioxane (12 mL) was added 1,3-bis[2,6-bis(1-propylbutyl)phenyl]-4,5-dichloro-2H-imidazol-1-ium-2-ide;3-chloropyridine;dichloropalladium (118.84 mg, 122.17 μmol, 0.05 eq) and Cs2CO3 (2.39 g, 7.33 mmol, 3 eq). The mixture was stirred at 100° C. for 12 h under N₂. LCMS showed starting material was consumed and one main peak with desired mass was detected. The reaction was added water (10 mL) and then extracted with EtOAc (10 mL×3). The combined organic phase was washed with brine (10 mL), dried with anhydrous Na2SO4, filtered and concentrated in vacuum. The residue was purified by column chromatography (SiO2, PE:EtOAc=0:1, Rf=0.4) to give compound tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate (660 mg, 55% yield) as a yellow gum.

LC-MS [ESI, M+1]: 490.3.

¹H NMR (400 MHz, CHLOROFORM-d) δ=7.63 (s, 1H), 7.40-7.34 (m, 2H), 7.33-7.28 (m, 2H), 7.27-7.21 (m, 1H), 6.93 (s, 1H), 5.22-5.04 (m, 1H), 4.04 (br s, 2H), 3.58 (d, J=8.8 Hz, 1H), 3.54-3.43 (m, 2H), 3.37 (dt, J=3.2, 8.4 Hz, 1H), 2.62 (s, 3H), 2.53 (br s, 2H), 2.45-2.32 (m, 3H), 2.29-2.20 (m, 1H), 1.51-1.43 (m, 12H).

### Step 5: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine

To a solution of tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate (100 mg, 204.25 μmol, 1 eq) in DCM (2 mL) was added TFA (1.54 g, 13.46 mmol, 1 mL, 65.91 eq). The mixture was stirred at 25° C. for 1 h. LCMS showed starting material was consumed and one main peak with desired mass was detected. The reaction mixture was concentrated under the reduced pressure to give the product. The pH was adjusted to around 7 by progressively NaHCO₃ (aq.), and then extracted with EtOAc (5 mL×3). The combined organic layers were washed with brine (5 mL), dried over Na2SO4, filtered and concentrated under reduced pressure to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (66 mg, 83% yield) as a yellow gum.

LC-MS [ESI, M+1]: 390.1.

¹H NMR (400 MHz, CHLOROFORM-d) δ=7.63 (d, J=1.6 Hz, 1H), 7.40-7.33 (m, 2H), 7.33-7.29 (m, 2H), 7.27-7.21 (m, 1H), 6.93 (s, 1H), 3.83 (s, 2H), 3.58 (d, J=8.8 Hz, 1H), 3.54-3.43 (m, 2H), 3.37 (dt, J=3.6, 8.8 Hz, 1H), 2.63 (s, 3H), 2.41-2.21 (m, 6H), 1.46 (s, 3H).

### Step 6: methyl (R)-6-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)amino)picolinate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (50 mg, 128.37 μmol, 1 eq) and methyl 6-fluoropyridine-2-carboxylate (19.91 mg, 128.37 μmol, 1 eq) in DMSO (1 mL) was added DIEA (49.77 mg, 385.12 μmol, 67.08 μL, 3 eq). The mixture was stirred at 120° C. for 1 h. LCMS showed starting material was consumed and the desired mass was detected. The reaction was added water (5 mL) and then extracted with ethyl acetate (10 mL×3).The combined organic phase was washed with brine (5 mL), dried with anhydrous Na2SO4, filtered and concentrated in vacuum. The residue was purified by prep-TLC (SiO2, PE:EtOAc=1:1, Rf=0.2) to give compound methyl 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]pyridine-2-carboxylate (25 mg, 37% yield) as a yellow gum.

LC-MS [ESI, M+1]: 525.2.

### Step 7: (R)-6-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)amino)picolinic acid

To a solution of methyl 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]pyridine-2-carboxylate (25 mg, 47.65 μmol, 1 eq) in THF (1 mL) was added LiOH·H₂O (6.00 mg, 142.96 μmol, 3 eq) in H₂O (0.3 mL). The mixture was stirred at 25° C. for 1 h. LCMS showed starting material was consumed and one main peak with desired mass was detected. The reaction mixture was poured into 2 mL water and acidified with 2M HCl to pH around 4 and then the reaction mixture was concentrated under the reduced pressure to give the product. The residue was purified by prep-HPLC (FA condition column: Phenomenex luna C18 150*25 mm*10 um; mobile phase: [water(FA)—ACN]; gradient:20%-50% B over 10 min) to give compound 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]pyridine-2-carboxylic acid (2.57 mg, 11% yield) as a white solid.

LC-MS [ESI, M+1]: 511.2.

¹H NMR (400 MHz, CHLOROFORM-d) δ=9.76 (br s, 1H), 7.82 (br t, J=7.6 Hz, 1H), 7.64 (br s, 1H), 7.52 (br d, J=6.8 Hz, 1H), 7.43-7.28 (m, 5H), 7.02 (br s, 1H), 6.83 (br d, J=8.4 Hz, 1H), 5.35 (s, 2H), 3.62 (br d, J=9.2 Hz, 1H), 3.58-3.47 (m, 2H), 3.45-3.38 (m, 1H), 3.20 (br d, J=13.2 Hz, 2H), 3.03 (br d, J=12.4 Hz, 2H), 2.65 (s, 3H), 2.47-2.35 (m, 1H), 2.33-2.23 (m, 1H), 1.48 (s, 3H).

### Example 47

(R)-6-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)carbamoyl)picolinic acid (Compound 466)

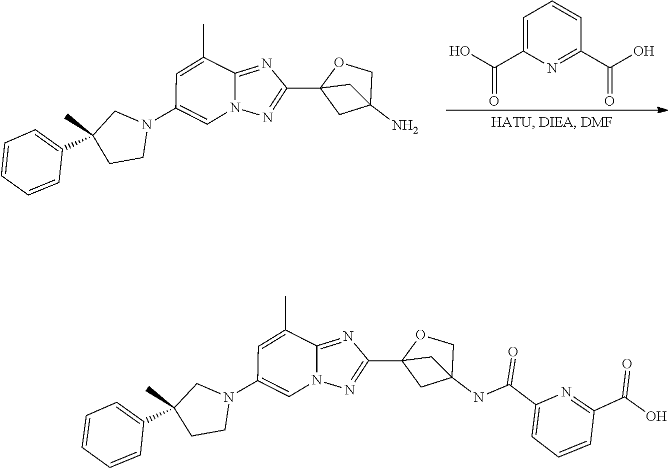

### Step 1: (R)-6-((1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)carbamoyl)picolinic acid

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (30 mg, 77.02 μmol, 1 eq) and pyridine-2,6-dicarboxylic acid (12.87 mg, 77.02 μmol, 1 eq) in DMF (1 mL) was added HATU (43.93 mg, 115.54 u mol, 1.5 eq). The mixture was stirred at 25° C. for 0.5 h, and then DIEA (49.77 mg, 385.12 u mol, 67.08 u L, 5 eq) was added at 25° C. The mixture was stirred at 25° C. for 1 h. LCMS showed starting material was consumed and one main peak with desired mass was detected. The reaction mixture was filtered. The residue was purified by prep-HPLC (FA condition column: Phenomenex luna C18 150*25 mm*10 um; mobile phase: [water(FA)—ACN]; gradient:38%-68% B over 8 min) to give compound 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamoyl]pyridine-2-carboxylic acid (25.99 mg, 63% yield) as a white solid.

LC-MS [ESI, M+1]: 539.1.

¹H NMR (400 MHz, CHLOROFORM-d) δ=9.13 (br s, 1H), 8.39 (dd, J=7.6, 17.2 Hz, 2H), 8.09 (t, J=7.6 Hz, 1H), 7.68 (s, 1H), 7.42-7.33 (m, 2H), 7.33-7.29 (m, 2H), 7.24 (br s, 1H), 7.10 (s, 1H), 4.25 (s, 2H), 3.61 (d, J=9.2 Hz, 1H), 3.57-3.48 (m, 2H), 3.41 (dt, J=3.6, 8.8 Hz, 1H), 2.85 (br d, J=4.0 Hz, 2H), 2.66 (s, 3H), 2.59 (br d, J=3.2 Hz, 2H), 2.43-2.36 (m, 1H), 2.31-2.24 (m, 1H), 1.47 (s, 3H).

| Compound | | |
|---|---|---|
| Number | Synthesis | LC/MS |
| 467 | Similar to | m/z 516.2 |
| | Example 47 | [M + H]⁺ |
| 468 | Similar to | m/z 516.2 |
| | Example 47 | [M + H]⁺ |

### Example 48

### 1-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)-2-oxopiperidine-4-carboxylic acid (Compound 469)

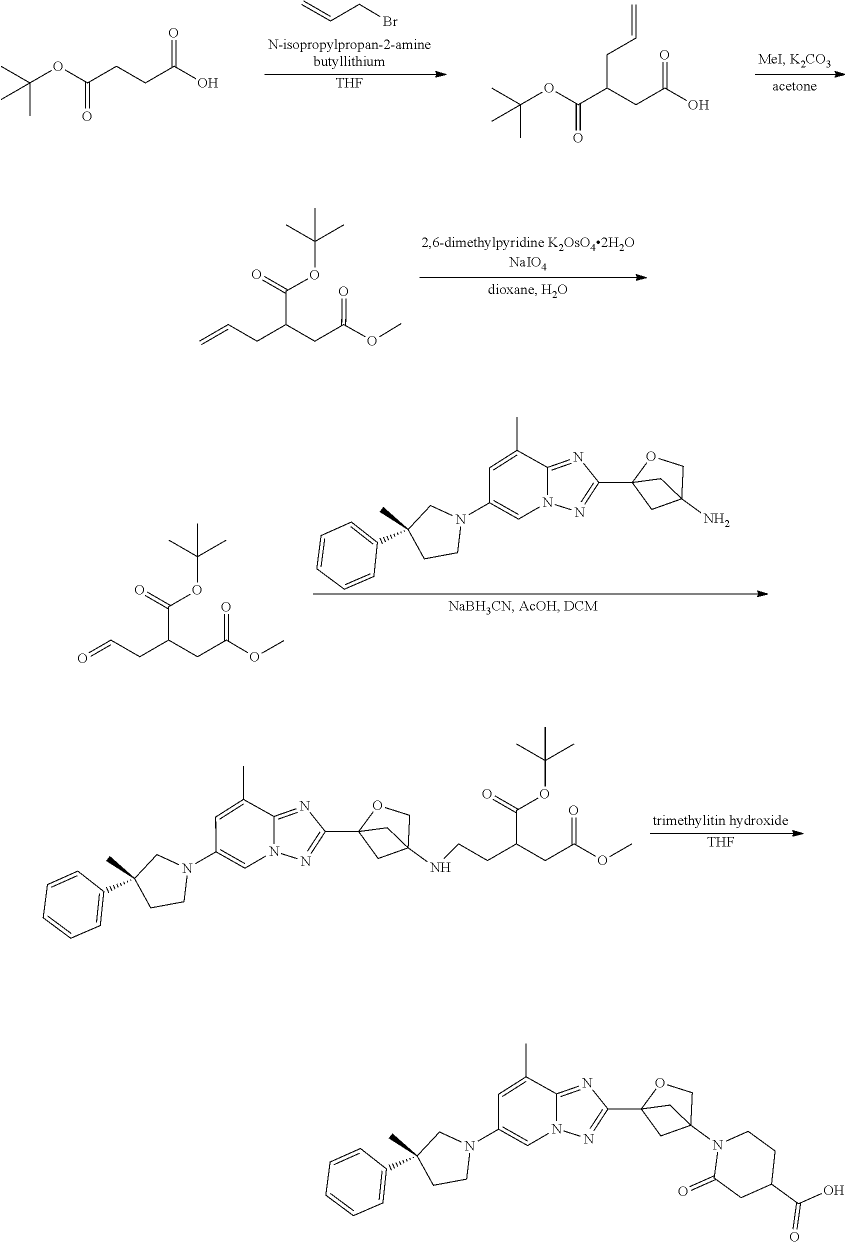

### Step 1: 3-(tert-butoxycarbonyl)hex-5-enoic acid

To a solution of N-isopropylpropan-2-amine (4.65 g, 45.93 mmol, 6.49 mL, 4 eq) in THF (20 mL) was added dropwise butyllithium (2.5 M, 18.37 mL, 4 eq) at −78° C. After addition, the mixture was stirred −78° C. for 15 min. Then 4-tert-butoxy-4-oxo-butanoic acid (2 g, 11.48 mmol, 1 eq) was added dropwise at −78° C., after 15 min 3-bromoprop-1-ene (6.94 g, 57.41 mmol, 5 eq) was added. The resulting mixture was stirred at −78° C. for 30 min, and then the mixture was stirred 0° C. for 30 min. TLC (SiO2, DCM:MeOH=10:1, Rf=0.5) indicated reactant was consumed completely and one new spot was formed. The reaction was quenched by 2M HCl (10 mL) slowly and then extracted with ethyl acetate (10 mL×3). The combined organic phase was washed with brine (10×3 mL), dried with anhydrous Na2SO4, filtered and concentrated in vacuum. The residue was purified by column chromatography (SiO2, DCM:MeOH=10:1,Rf=0.5) to give compound 3-tert-butoxycarbonylhex-5-enoic acid (1.1 g, 45% yield) as a colorless liquid.

¹H NMR (400 MHz, CHLOROFORM-d) δ=5.73 (tdd, J=7.2, 10.1, 17.2 Hz, 1H), 5.18-5.02 (m, 2H), 2.88-2.76 (m, 1H), 2.74-2.63 (m, 1H), 2.52-2.36 (m, 2H), 2.35-2.21 (m, 1H), 1.44 (s, 9H).

### Step 2: 1-(tert-butyl) 4-methyl 2-allylsuccinate

To a solution of 3-tert-butoxycarbonylhex-5-enoic acid (1.1 g, 5.13 mmol, 1 eq) and K₂CO₃ (3.55 g, 25.67 mmol, 5 eq) in ACETONE (55 mL) was added dropwise CH₃I (1.46 g, 10.27 mmol, 639.23 μL, 2 eq) at 0° C. The mixture was stirred at 25° C. for 1 h. TLC (SiO2, PE:EtOAc=3:1, Rf=0.36) indicated reactant was consumed and one new spot formed. The reaction was quenched by ice water (10 mL) slowly and then extracted with ethyl acetate (10 mL×3).The combined organic phase was washed with brine (10 mL), dried with anhydrous Na2SO4, filtered and concentrated in vacuum. The residue was purified by column chromatography (SiO2, PE:EtOAc=3:1, Rf=0.36) to give compound O1-tert-butyl O4-methyl 2-allylbutanedioate (1.1 g, 94% yield) as a light yellow liquid.

¹H NMR (400 MHz, CHLOROFORM-d) δ=5.73 (tdd, J=7.2, 10.0, 17.2 Hz, 1H), 5.18-5.00 (m, 2H), 3.68 (s, 3H), 2.88-2.77 (m, 1H), 2.69-2.59 (m, 1H), 2.46-2.35 (m, 2H), 2.32-2.22 (m, 1H), 1.45 (s, 9H).

### Step 3: 1-(tert-butyl) 4-methyl 2-(2-oxoethyl)succinate

To a solution of O1-tert-butyl O4-methyl 2-allylbutanedioate (200 mg, 876.10 μmol, 1 eq) and 2,6-dimethylpyridine (187.75 mg, 1.75 mmol, 204.08 μL, 2 eq) in dioxane (4 mL) and H₂O (4 mL) was added K₂OSO₄·2H₂O (32.28 mg, 87.61 μmol, 0.1 eq) and NaIO₄ (749.56 mg, 3.50 mmol, 194.19 μL, 4 eq) at 0° C. The mixture was stirred at 25° C. for 12 h. TLC (SiO2, PE:EtOAc=3:1) indicated reactant was consumed completely and some new spots formed. The reaction was quenched by aq. Na₂S₂O₃ (5 mL) slowly and then extracted with ethyl acetate (5 mL×3). The combined organic phase was washed with brine (5 mL), dried with anhydrous Na₂SO₄, filtered and concentrated in vacuum. The residue was purified by column chromatography (SiO₂, PE:EtOAc=3:1, Rf=0.7) to give compound O1-tert-butyl O4-methyl 2-(2-oxoethyl)butanedioate (36 mg, 18% yield) as a colorless liquid.

¹H NMR (400 MHz, CHLOROFORM-d) δ=9.75 (s, 1H), 3.68 (d, J=1.2 Hz, 3H), 3.30-3.17 (m, 1H), 2.94-2.80 (m, 1H), 2.68 (dt, J=6.4, 17.2 Hz, 2H), 2.56-2.50 (m, 1H), 1.42 (d, J=1.6 Hz, 9H).

### Step 4: 1-(tert-butyl) 4-methyl 2-(2-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)amino)ethyl)succinate

To a solution of O1-tert-butyl O4-methyl 2-(2-oxoethyl)butanedioate (35.47 mg, 154.05 μmol, 2 eq) in DCM (1 mL) was added 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (30 mg, 77.02 μmol, 1 eq), AcOH (462.53 μg, 7.70 μmol, 0.1 eq) and NaBH₃CN (14.52 mg, 231.07 μmol, 3 eq). The mixture was stirred at 25° C. for 1 h. LCMS showed starting material was consumed and the desired mass was detected. The reaction mixture was concentrated under the reduced pressure to give the product. The reaction was added water (5 mL) and then extracted with ethyl acetate (5 mL×3).The combined organic phase was washed with brine (5 mL), dried with anhydrous Na₂SO₄, filtered and concentrated in vacuum to give compound 01-tert-butyl O4-methyl 2-[2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]ethyl]butanedioate (60 mg, crude) as a white gum.

LC-MS [ESI, M+1]: 604.4.

### Step 5: tert-butyl 1-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)-2-oxopiperidine-4-carboxylate

To a solution of O1-tert-butyl O4-methyl 2-[2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]ethyl]butanedioate (10 mg, 16.56 μmol, 1 eq) in THF (0.5 mL) was added trimethyltin hydroxide (1.79 mg, 33.13 μmol, 2 eq). The mixture was stirred at 25° C. for 1 h. LCMS showed starting material was consumed and one main peak with desired mass was detected. The reaction mixture was concentrated under the reduced pressure to give the product. The residue was purified by prep-HPLC (FA condition column: Waters xbridge 150*25 mm 10 um; mobile phase: [water(FA)—ACN]; gradient:44%-74% B over 10 min) to give compound 1-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-2-oxo-piperidine-4-carboxylic acid (2.26 mg, 27% yield) as a white solid.

LC-MS [ESI, M+1]: 516.1.

¹H NMR (400 MHz, CHLOROFORM-d) δ=7.67 (d, J=1.6 Hz, 1H), 7.41-7.35 (m, 2H), 7.34-7.30 (m, 2H), 7.27 (br s, 1H), 6.97 (s, 1H), 4.19-3.97 (m, 2H), 3.60 (d, J=8.8 Hz, 1H), 3.57-3.46 (m, 3H), 3.45-3.35 (m, 2H), 3.02-2.89 (m, 1H), 2.82-2.63 (m, 6H), 2.59-2.49 (m, 3H), 2.41-2.36 (m, 1H), 2.30-2.19 (m, 3H), 1.48 (s, 3H).

### Example 49

### 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylic acid (Compound 470)

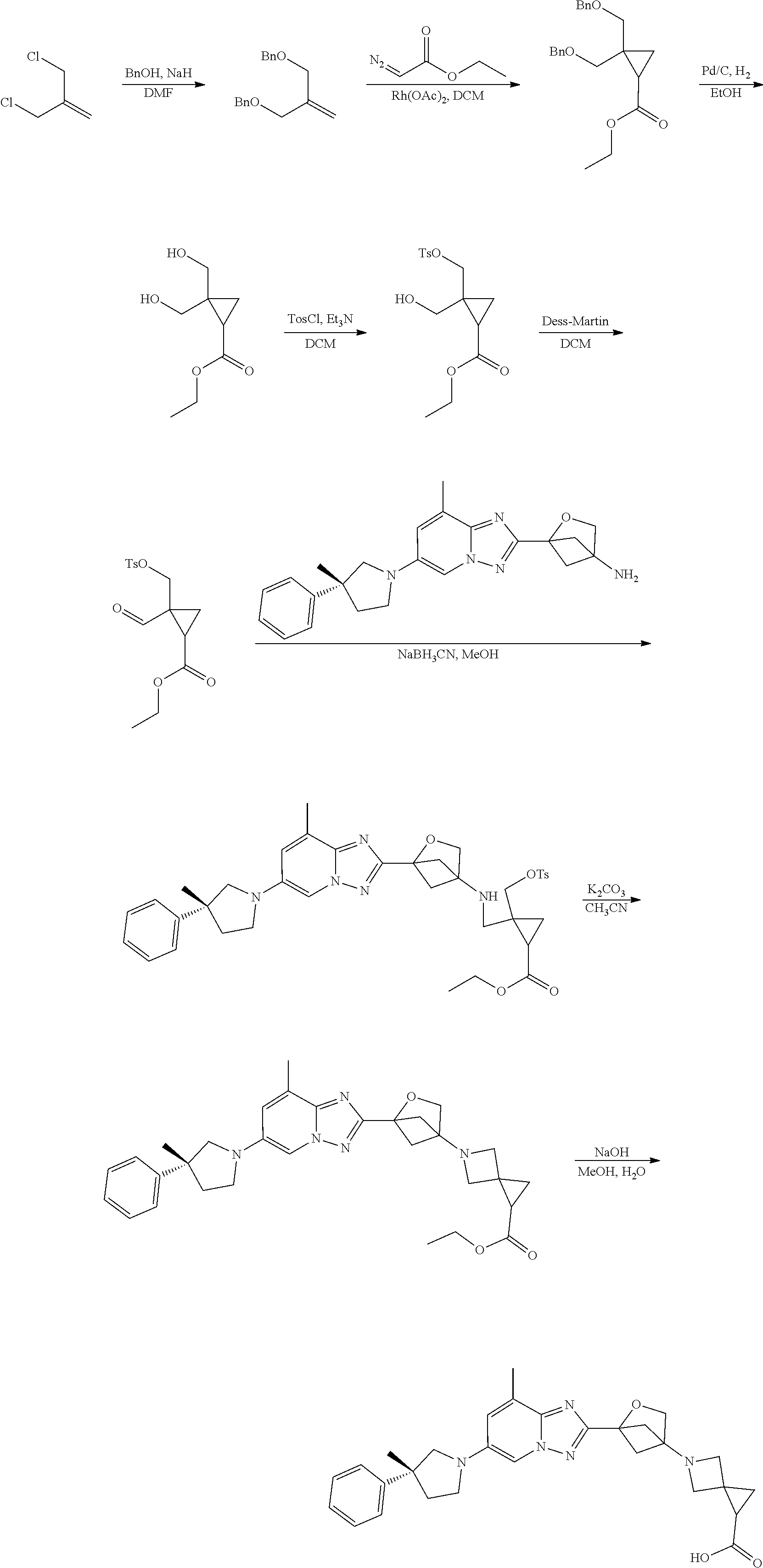

### Step 1: 2-(benzyloxymethyl)allyloxymethylbenzene

NaH (4.00 g, 100.00 mmol, 60% purity, 2.5 eq) in DMF (200 mL) was stirred at 0° C. and the BnOH (10.81 g, 100.00 mmol, 10.36 mL, 2.5 eq) was added dropwise at 0° C. under N₂. The mixture was stirred at 0° C. for 20 min. Then the cooling bath was removed and the mixture was stirred at 25° C. for 1 h under N₂. Then to the mixture was added 3-chloro-2-(chloromethyl)prop-1-ene (5 g, 40.00 mmol, 4.63 mL, 1 eq) and the reaction was stirred at 25° C. for 14 h under N₂. LCMS showed reactant was consumed completely and one main peak with desired mass was detected. The reaction mixture was quenched by addition NH₄Cl (150 mL), and extracted with EtOAc (200 mL×3). The combined organic layer was dried over anhydrous Na₂SO₄, filtered and the filtrate was concentrated under reduced pressure to give a residue. The residue was purified by column chromatography (SiO₂, PE:EtOAc=20:1, Rf=0.6) to give 2-(benzyloxymethyl)allyloxymethylbenzene (9.2 g, 33.26 mmol, 83.14% yield, 97% purity) as yellow oil.

LC-MS [ESI, M+23]: 291.2

¹H NMR (400 MHz, CHLOROFORM-d) δ=7.39-7.33 (m, 8H), 7.33-7.27 (m, 2H), 5.28 (s, 2H), 4.53 (s, 4H), 4.09 (s, 4H).

### Step 2: ethyl 2,2-bis(benzyloxymethyl)cyclopropanecarboxylate

To a solution of 2-(benzyloxymethyl)allyloxymethylbenzene (8.2 g, 30.56 mmol, 1 eq) and Rh(OAc)₂ (675.28 mg, 3.06 mmol, 0.1 eq) in DCM (150 mL) was added ethyl 2-diazoacetate (17.43 g, 152.79 mmol, 16.07 mL, 5 eq) in DCM (10 mL) dropwise slowly at 0° C. under N₂. The mixture was stirred at 25° C. for 16 h. TLC (PE:EtOAc=10:1, Rf=0.6) indicated reactant 1 remained and three new spots formed. The reaction mixture was quenched by addition HCl (1 M, 20 mL). Then the reaction was diluted with H₂O (50 mL) and extracted with DCM (70 mL×3). The combined organic layers were washed with brine (60 mL×2), dried over Na₂SO₄, filtered and the filtrate was concentrated under reduced pressure to give a residue. The residue was purified by column chromatography (SiO₂, PE:EtOAc=10:1, Rf=0.5) to give ethyl 2,2-bis(benzyloxymethyl)cyclopropanecarboxylate (7.8 g, 22.01 mmol, 72.02% yield) as yellow oil.

¹H NMR (400 MHz, CHLOROFORM-d) δ=7.38-7.26 (m, 10H), 4.51 (d, J=2.0 Hz, 2H), 4.44 (d, J=4.0 Hz, 2H), 4.17-4.06 (m, 2H), 3.89 (d, J=9.6 Hz, 1H), 3.74 (d, J=9.6 Hz, 1H), 3.59 (d, J=9.6 Hz, 1H), 3.31 (d, J=9.6 Hz, 1H), 1.78 (dd, J=5.6, 8.0 Hz, 1H), 1.29-1.18 (m, 4H), 1.12 (dd, J=4.8, 8.0 Hz, 1H).

### Step 3: ethyl 2,2-bis(hydroxymethyl)cyclopropanecarboxylate

Solution 1: {ethyl 2,2-bis(benzyloxymethyl)cyclopropanecarboxylate, 1 eq, 0.4 g} in {EtOH, 10 mL}. The fixed bed (named FLR1, volume 5 mL) was completely packed with granular catalyst WXC1030, 30 g, 5% Pd/C, N/A eq. The H2 back pressure regulator was adjusted to 1 MPa, and the flow rate of H2 was 30 mL/min. Then the solution S1 was pumped by Pump 1 {S1,P1,0.3 mL/min} to fixed bed {FLR1,SS,Fixed bed, 6.350 (¼″) mm, 1 mL, 50° C.}.

The solution S1 was flowing through {FLR1,3.333 min to leave the reactor zone, then the reaction mixture was collected with a bottle at 20° C. TLC (PE:EtOAc=10:1, Rf=0.5) indicated reactant 1 consumed, and one major new spot with larger polarity was detected. The suspension was filtered through a pad of celite to give ethyl 2,2-bis(hydroxymethyl)cyclopropanecarboxylate (5.2 g, crude) as yellow oil.

¹H NMR (400 MHz, CHLOROFORM-d) δ=4.16-4.13 (m, 2H), 4.09 (d, J=11.6 Hz, 1H), 3.97-3.86 (m, 1H), 3.77 (d, J=11.2 Hz, 1H), 3.49 (d, J=11.2 Hz, 1H), 1.83-1.79 (m, 1H), 1.27-1.24 (m, 4H), 1.05 (dd, J=4.8, 8.4 Hz, 1H).

### Step 4: ethyl 2-(hydroxymethyl)-2-(p-tolylsulfonyloxymethyl)cyclopropanecarboxylate

To a solution of ethyl 2,2-bis(hydroxymethyl)cyclopropanecarboxylate (2 g, 11.48 mmol, 1 eq), TEA (3.49 g, 34.44 mmol, 4.79 mL, 3 eq) and DMAP (140.27 mg, 1.15 mmol, 0.1 eq) in DCM (30 mL) was added TosCl (1.09 g, 5.74 mmol, 0.5 eq) at 0° C. The mixture was stirred at 25° C. for 2 h. LCMS showed reactant consumed and desired mass was detected. The reaction was diluted with H₂O (50 mL) and extracted with DCM (80 mL×3). The combined organic layers were washed with brine (80 mL×2), dried over Na₂SO₄, filtered and the filtrate was concentrated under reduced pressure to give a residue. The residue was purified by column chromatography (SiO₂, PE:EtOAc=3:1, Rf=0.6) to give ethyl 2-(hydroxymethyl)-2-(p-tolylsulfonyloxymethyl)cyclopropanecarboxylate (520 mg, 1.58 mmol, 13.79% yield) as yellow oil.

LC-MS [ESI, M+1]: 329.1

¹H NMR (400 MHz, CHLOROFORM-d) δ=7.80 (d, J=8.4 Hz, 2H), 7.37 (d, J=8.0 Hz, 2H), 4.28-4.07 (m, 4H), 3.94 (d, J=12.0 Hz, 1H), 3.84 (d, J=10.4 Hz, 1H), 3.72 (d, J=12.0 Hz, 1H), 2.49-2.46 (m, 3H), 1.73 (dd, J=6.0, 8.4 Hz, 1H), 1.34 (t, J=5.6 Hz, 1H), 1.30-1.25 (m, 3H), 1.10 (dd, J=5.2, 8.8 Hz, 1H).

### Step 5: ethyl 2-formyl-2-(p-tolylsulfonyloxymethyl)cyclopropanecarboxylate

To a solution of ethyl 2-(hydroxymethyl)-2-(p-tolylsulfonyloxymethyl)cyclopropanecarboxylate (250 mg, 761.31 μmol, 1 eq) in DCM (6 mL) was added DMP (484.36 mg, 1.14 mmol, 353.80 μL, 1.5 eq) at 0° C. The mixture was stirred at 25° C. for 1 hr. TLC (PE:EtOAc=3:1, Rf=0.6) indicated reactant 1 remained and one new spot formed. The reaction was filtered and the filtrate was diluted with H₂O (10 mL) and extracted with DCM (15 mL×3). The combined organic layers were washed with brine (15 mL×2), dried over Na₂SO₄, filtered and the filtrate was concentrated under reduced pressure to give a residue. The residue was purified by column chromatography (SiO₂, PE:EtOAc=3:1, Rf=0.5) to give ethyl 2-formyl-2-(p-tolylsulfonyloxymethyl)cyclopropanecarboxylate (200 mg, 612.81 μmol, 80.49% yield) as yellow oil.

¹H NMR (400 MHz, CHLOROFORM-d) δ=9.30 (s, 1H), 7.80 (d, J=8.0 Hz, 2H), 7.38 (d, J=8.4 Hz, 2H), 4.28 (d, J=10.4 Hz, 1H), 4.21-4.13 (m, 3H), 2.47 (s, 3H), 2.38-2.29 (m, 1H), 1.70 (dd, J=5.6, 8.4 Hz, 1H), 1.30-1.26 (m, 4H).

### Step 6: ethyl 2-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]methyl]-2-(p-tolylsulfonyloxymethyl)cyclopropanecarboxylate

To a solution of ethyl 2-formyl-2-(p-tolylsulfonyloxymethyl)cyclopropanecarboxylate (192.23 mg, 500.65 μmol, 1.5 eq) and 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (130 mg, 333.77 μmol, 1 eq) in MeOH (3 mL) was added NaBH₃CN (41.95 mg, 667.54 μmol, 2 eq). The mixture was stirred at 25° C. for 12 hr. LCMS showed reactant 1 was consumed completely and desired mass was detected. The reaction was concentrated under reduced pressure to give a residue. The residue t was purified by reversed-phase HPLC (0.1% FA condition) to give ethyl 2-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]methyl]-2-(p-tolylsulfonyloxymethyl)cyclopropanecarboxylate (80 mg, 103.68 u mol, 31.06% yield, 90.699% purity) as yellow oil.

LC-MS [ESI, M+1]: 700.4

### Step 7: ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylate

To a solution of ethyl 2-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]methyl]-2-(p-tolylsulfonyloxymethyl)cyclopropanecarboxylate (80 mg, 114.31 μmol, 1 eq) in MeCN (3 mL) was added K₂CO₃ (63.19 mg, 457.24 μmol, 4 eq). The mixture was stirred at 70° C. for 16 h. LCMS showed reactant was consumed completely and 62% of desired mass was detected. The reaction was concentrated under reduced pressure to give a residue. The residue was purified by reversed-phase HPLC (0.1% FA condition) to give ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylate (28 mg, 52.10 u mol, 45.58% yield, 98.181% purity) as yellow oil.

LC-MS [ESI, M+1]: 528.4

### Step 8: 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylic acid

To a solution of ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylate (23 mg, 43.59 μmol, 1 eq) in THF (0.8 mL) was added NaOH (5.23 mg, 130.77 μmol, 3 eq) in water (0.8 mL). The mixture was stirred at 25° C. for 0.5 h. LCMS showed reactant was consumed completely and one main peak with desired mass was detected. The reaction was concentrated under reduced pressure to give a residue. The residue was purified by prep-HPLC (column: CD24-WePure Biotech XPT C18 150×25×7 um; mobile phase: [H2O (10 mM NH₄HCO₃)—ACN]; gradient:22%-52% B over 11.0 min) to give 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-5-azaspiro[2.3]hexane-2-carboxylic acid (10.41 mg, 20.68 μmol, 47.45% yield, 99.254% purity) as a white solid.

LC-MS [ESI, M+1]: 500.3

¹H NMR (400 MHz, METHANOL-d4) δ=7.72 (d, J=1.6 Hz, 1H), 7.43-7.27 (m, 4H), 7.25-7.16 (m, 2H), 4.44-4.26 (m, 2H), 3.80 (s, 2H), 3.61-3.51 (m, 3H), 3.40 (dt, J=3.6, 8.8 Hz, 1H), 3.03 (q, J=12.8 Hz, 2H), 2.58 (s, 3H), 2.42-2.24 (m, 4H), 2.16-2.02 (m, 3H), 1.51-1.40 (m, 4H), 1.13-1.05 (m, 1H).

### Example 50

### (R)-1-(8-methyl-6-(3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexan-4-amine (Compound 471)

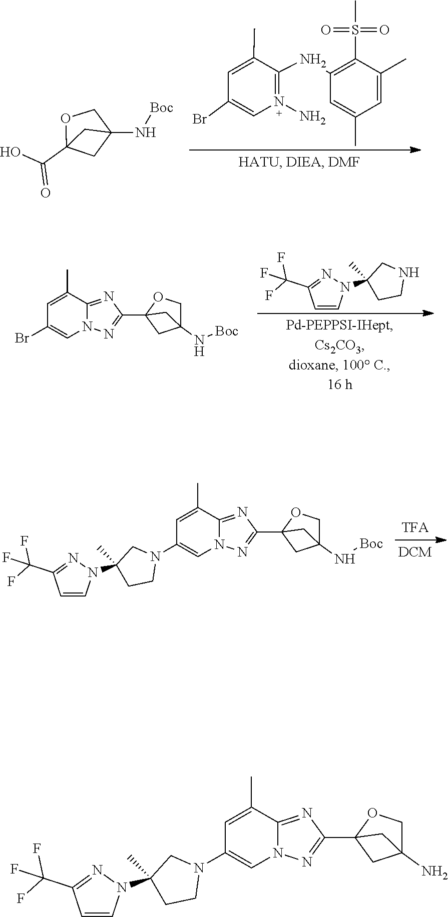

### Step 1: tert-butyl (1-(6-bromo-8-methyl-[1,2,4]triazolo[1,5-a]pyridin-2-yl)-2-oxabicyclo[2.1.1]hexan-4-yl)carbamate

To a solution of 4-(tert-butoxycarbonylamino)-2-oxabicyclo[2.1.1]hexane-1-carboxylic acid (500 mg, 2.06 mmol, 1 eq) and 5-bromo-3-methyl-pyridin-1-ium-1,2-diamine;2,4,6-trimethylbenzenesulfonate (1.65 g, 4.11 mmol, 2 eq) in DMF (6 mL) was added HATU (1.17 g, 3.08 mmol, 1.5 eq). The mixture was stirred at 25° C. for 0.5 h, and then DIEA (1.33 g, 10.28 mmol, 1.79 mL, 5 eq) was added at 25° C. The mixture was stirred at 25° C. for 1 h. LCMS showed starting material was consumed and one main peak with desired mass was detected. The reaction was added water (10 mL) and then extracted with ethyl acetate (10 mL×3). The combined organic phase was washed with brine (5 mL), dried with anhydrous Na₂SO₄, filtered and concentrated in vacuum. The residue was purified by column chromatography (SiO₂, PE:EtOAc=1:1, Rf=0.2) to give compound tert-butyl N-[1-(6-bromo-8-methyl-[1,2,4]triazolo[1,5-a]pyridin-2-yl)-2-oxabicyclo[2.1.1]hexan-4-yl]carbamate (1 g, crude) as a white solid.

LC-MS [ESI, M+1]: 409.0.

¹H NMR (400 MHz, CHLOROFORM-d) δ=8.54 (s, 1H), 7.39 (s, 1H), 4.05 (br s, 2H), 2.70-2.49 (m, 5H), 2.40 (br d, J=4.4 Hz, 2H), 1.48 (s, 9H).

### Step 2: tert-butyl (R)-(1-(8-methyl-6-(3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexan-4-yl)carbamate

To a solution of tert-butyl N-[1-(6-bromo-8-methyl-[1,2,4]triazolo[1,5-a]pyridin-2-yl)-2-oxabicyclo[2.1.1]hexan-4-yl]carbamate (100.82 mg, 246.34 μmol, 1.2 eq) and 1-[(3R)-3-methylpyrrolidin-3-yl]-3-(trifluoromethyl)pyrazole (45 mg, 205.29 μmol, 1 eq) in dioxane (3 mL) was added [1,3-bis[2,6-bis(1-ethylpropyl)phenyl]-4,5-dichloro-imidazol-2-ylidene]-dichloro-(2-methylpyridin-1-ium-1-yl)palladium (8.62 mg, 10.26 μmol, 0.05 eq) and Cs₂CO₃ (200.66 mg, 615.86 μmol, 3 eq). The mixture was stirred at 100° C. for 12 h under N₂. LCMS showed starting material was consumed and one main peak with desired mass was detected. The reaction mixture was filtered and the filtrate was concentrated under the reduced pressure to give the product. The crude product was purified by reversed-phase HPLC (0.1% FA condition) to give compound tert-butyl N-[1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexan-4-yl]carbamate (40 mg, 36% yield) as a yellow gum.

LC-MS [ESI, M+1]: 548.3.

### Step 3: (R)-1-(8-methyl-6-(3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexan-4-amine

To a solution of tert-butyl N-[1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexan-4-yl]carbamate (40 mg, 73.05 μmol, 1 eq) in DCM (1 mL) was added TFA (767.50 mg, 6.73 mmol, 0.5 mL, 92.14 eq). The mixture was stirred at 25° C. for 0.5 h. LCMS showed starting material was consumed and one main peak with desired mass was detected. The reaction was added water (10 mL) and then extracted with ethyl acetate (10 mL×3). The combined organic phase was washed with brine (5 mL), dried with anhydrous Na₂SO₄, filtered and concentrated in vacuum. The residue was purified by prep-HPLC (NH₄HCO₃ condition column: CD24-WePure Biotech XPT C18 150*25*7 um; mobile phase: [H₂O (10 mM NH₄HCO₃)—ACN]; gradient:20%-50% B over 10.0 min) to give compound 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexan-4-amine (20.7 mg, 62% yield) as a white solid.

LC-MS [ESI, M+1]: 448.1.

¹H NMR (400 MHz, CHLOROFORM-d) δ=7.62 (s, 1H), 7.55 (d, J=0.8 Hz, 1H), 6.89 (s, 1H), 6.53 (d, J=2.0 Hz, 1H), 3.97 (d, J=10.0 Hz, 1H), 3.82 (s, 2H), 3.58 (d, J=10.0 Hz, 1H), 3.46 (t, J=7.2 Hz, 2H), 2.84 (td, J=6.4, 13.2 Hz, 1H), 2.62 (s, 3H), 2.42-2.32 (m, 1H), 2.31-2.22 (m, 4H), 1.80 (s, 3H).

### Example 51

### (R)-1-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)piperidine-4-carboxylic acid (Compound

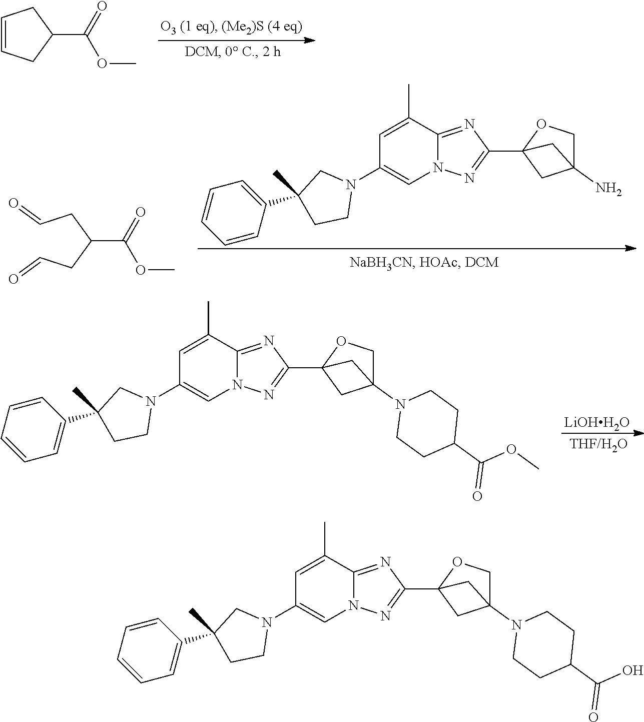

### Step 1: methyl 4-oxo-2-(2-oxoethyl)butanoate

A stream of OZONE (380.48 mg, 7.93 mmol, 1 eq) is passed through a cooled −78° C. solution of methyl cyclopent-3-ene-1-carboxylate (1 g, 7.93 mmol, 1 eq) in DCM (20 mL) until TLC analysis (PE:EtOAc=5:1) indicates that conversion of starting material (Rf=0.50) to a more polar product is complete. The reaction mixture is then quenched with Me₂S (1.97 g, 31.71 mmol, 2.33 mL, 4 eq) stirred at −20° C. and stirred at 25° for 1 h. TLC (SiO₂, PE:EtOAc=5:1, Rf=0.5) indicated reactant was consumed completely and one new spot was formed. The mixture was concentrated in vaco to give compound methyl 4-oxo-2-(2-oxoethyl)butanoate (1.3 g, crude) as a colourless gum.

¹H NMR (400 MHz, CHLOROFORM-d) δ=9.81-9.66 (m, 2H), 3.77 (s, 3H), 2.64 (s, 5H).

### Step 2: methyl (R)-1-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)piperidine-4-carboxylate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (20 mg, 51.35 μmol, 1 eq) in DCM (1 mL) was added methyl 4-oxo-2-(2-oxoethyl)butanoate (8.12 mg, 51.35 μmol, 1 eq), AcOH (308.36 g, 5.13 μmol, 0.1 eq) and NaBH₃CN (9.68 mg, 154.05 μmol, 3 eq) at 0° C. The mixture was stirred at 25° C. for 1 h. LCMS showed starting material was consumed and the desired mass was detected. The reaction mixture was concentrated under the reduced pressure to give the product. The reaction was added water (5 mL) and then extracted with ethyl acetate (10 mL×3).The combined organic phase was washed with brine (10 mL), dried with anhydrous Na₂SO₄, filtered and concentrated in vacuum. The residue was purified by prep-TLC (SiO₂, PE:EtOAc=0:1, Rf=0.6) to give compound methyl 1-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]piperidine-4-carboxylate (18 mg, 68% yield) was obtained as a yellow solid

LC-MS [ESI, M+1]: 516.4.

¹H NMR (400 MHz, CHLOROFORM-d) δ=7.59 (s, 1H), 7.27 (s, 5H), 6.89 (s, 1H), 3.90 (s, 2H), 3.66 (s, 3H), 3.54 (d, J=8.8 Hz, 1H), 3.49-3.39 (m, 2H), 3.33 (dt, J=3.6, 8.8 Hz, 1H), 3.05-2.97 (m, 2H), 2.58 (s, 3H), 2.40-2.28 (m, 6H), 2.25-2.08 (m, 4H), 1.92 (br d, J=2.4 Hz, 1H), 1.81-1.75 (m, 2H), 1.41 (s, 3H).

### Step 3: (R)-1-(1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)piperidine-4-carboxylic acid

To a solution of methyl 1-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]piperidine-4-carboxylate (18 mg, 34.91 μmol, 1 eq) in THF (0.5 mL) was added LiOH·H₂O (5.86 mg, 139.63 μmol, 4 eq) in H₂O (0.2 mL). The mixture was stirred at 25° C. for 1 h. LCMS showed starting material was consumed and the desired mass was detected. The reaction mixture was poured into 3 mL water and acidified with 2M HCl to pH around 4 and then the reaction mixture was concentrated under the reduced pressure to give the product. The residue was purified by prep-HPLC (FA condition column: CD01-Phenomenex luna C18 150*25*10 um; mobile phase: [water(FA)—ACN]; gradient:16%-46% B over 10 min) to give compound 1-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]piperidine-4-carboxylic acid (4.71 mg, 27% yield) as a yellow solid.

LC-MS [ESI, M+1]: 502.2

¹H NMR (400 MHz, DMSO-d₆) δ=7.86 (s, 1H), 7.43-7.30 (m, 4H), 7.27-7.19 (m, 2H), 3.73 (s, 2H), 3.60-3.41 (m, 6H), 2.94 (br d, J=10.9 Hz, 2H), 2.59-2.52 (m, 1H), 2.34-2.15 (m, 7H), 1.96 (br d, J=3.9 Hz, 2H), 1.84 (br d, J=11.5 Hz, 2H), 1.55 (q, J=10.9 Hz, 2H), 1.35 (s, 3H).

### Example 52

### (R)-(3,3-dimethyl-1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanol (Compound 473)

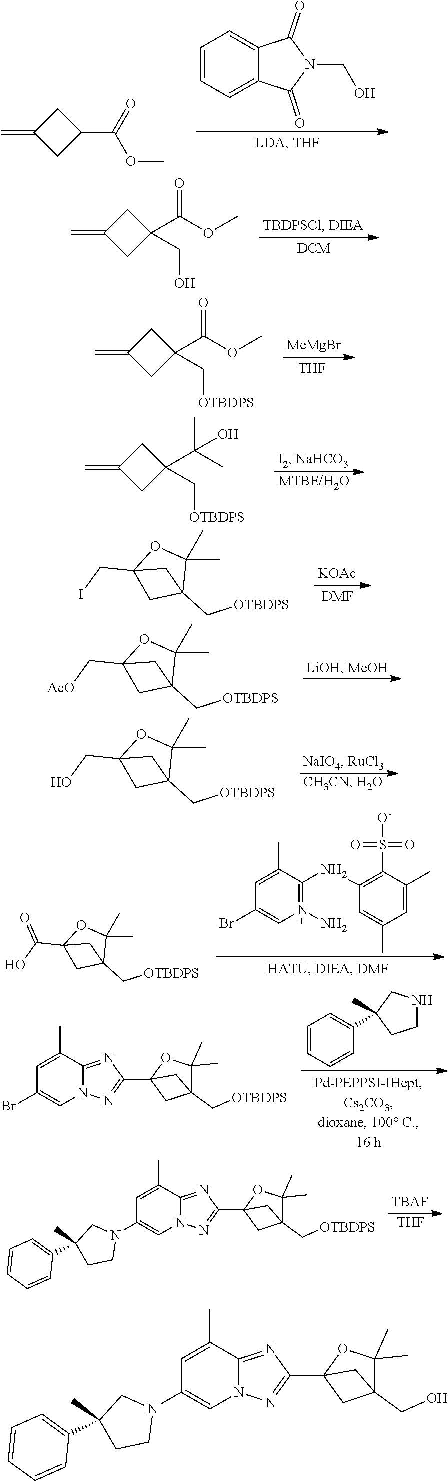

### Step 1: methyl 1-(hydroxymethyl)-3-methylenecyclobutane-1-carboxylate

To a stirred solution of butyllithium (2.5 M, 9.51 mL, 3 eq) in anhydrous THF (20 mL) was added dropwise N isopropylpropan-2-amine (2.41 g, 23.78 mmol, 3.36 mL, 3 eq) at −10° C. The solution was allowed to stirred at 0° C. for 30 minutes then cooled to −78° C. Methyl 3-methylenecyclobutanecarboxylate (1 g, 7.93 mmol, 1 eq) in anhydrous THF (4 mL) was added dropwise and stirred for an additional 1 h. Benzotriazol-1-ylmethanol (2.36 g, 15.85 mmol, 2 eq) in anhydrous THF (16 mL) was added dropwise over a 30 minutes period and kept 2 h at this temperature. TLC (SiO₂, PE:EtOAc=2:1) indicated reactant was consumed completely and one new spot was formed. The reaction mixture was quenched with saturated NH₄Cl (20 mL) at −20° C. After that, the resulting mixture was extracted with ethyl acetate (50 mL×2). The combined organic phase was washed with brine (80 mL), dried with anhydrous Na₂SO₄, filtered and concentrated in vacuum. The residue was purified by column chromatography (SiO₂, PE:EtOAc=2:1, Rf=0.4) to give compound methyl 1-(hydroxymethyl)-3-methylene-cyclobutanecarboxylate (2 g, crude) as yellow oil.

¹H NMR (400 MHz, CHLOROFORM-d) δ=4.90 (quin, J=2.4 Hz, 2H), 3.82 (s, 2H), 3.77 (s, 3H), 3.15-3.06 (m, 2H), 2.67-2.58 (m, 2H).

### Step 2: methyl 1-(((tert-butyldiphenylsilyl)oxy)methyl)-3-methylenecyclobutane-1-carboxylate

To a solution of methyl 1-(hydroxymethyl)-3-methylene-cyclobutanecarboxylate (2 g, 12.81 mmol, 1 eq) in DCM (10 mL) was added TBDPSCl (3.52 g, 12.81 mmol, 3.28 mL, 1 eq) and imidazole (1.82 g, 26.74 mmol, 2.45 mL, 2.09 eq) at 0-25° C. The mixture was stirred at 0-25° C. for 1 h. TLC (SiO₂, PE:EtOAc=6:1) indicated reactant was consumed completely and one new spot was formed. The reaction mixture was filtered and the filtrate was concentrated under the reduced pressure to give the product. The residue was purified by column chromatography (SiO₂, PE:EtOAc=6:1, Rf=0.7) to give compound methyl 1-[[tert-butyl(diphenyl)silyl]oxymethyl]-3-methylene-cyclobutanecarboxylate (700 mg, 1.76 mmol, 13.71% yield, 99% purity) as colorless oil.

¹H NMR (400 MHz, CHLOROFORM-d) δ=7.71-7.59 (m, 4H), 7.49-7.33 (m, 6H), 4.83 (quin, J=2.4 Hz, 2H), 3.91 (s, 2H), 3.71 (s, 3H), 3.13-3.02 (m, 2H), 2.66 (qd, J=2.0, 17.2 Hz, 2H), 1.04 (s, 9H).

### Step 3: 2-(1-(((tert-butyldiphenylsilyl)oxy)methyl)-3-methylenecyclobutyl)propan-2-ol

To a mixture of methyl 1-[[tert-butyl(diphenyl)silyl]oxymethyl]-3-methylene-cyclobutanecarboxylate (700 mg, 1.77 mmol, 1 eq) in THF (10 mL) was degassed and purged with N₂ for 3 times, MeMgBr (3 M, 1.77 mL, 3 eq) was added dropwise at 0° C. The mixture was stirred at 0° C. for 1 h under N₂ atmosphere. TLC (SiO₂, PE:EtOAc=6:1) indicated reactant was consumed completely and one new spot was formed. The reaction mixture was quenched with saturated NH₄Cl (30 mL) at 0° C. After that, the resulting mixture was extracted with ethyl acetate (50 mL×2). The combined organic phase was washed with brine (30 mL), dried with anhydrous Na₂SO₄, filtered and filtered and concentrated in vacuo to give compound 2-[1-[[tert-butyl(diphenyl)silyl]oxymethyl]-3-methylene-cyclobutyl]propan-2-ol (680 mg, 1.72 mmol, 97.13% yield) as yellow oil.

¹H NMR (400 MHz, CHLOROFORM-d) δ=7.68 (dd, J=1.6, 8.0 Hz, 4H), 7.50-7.38 (m, 6H), 4.81-4.73 (m, 2H), 3.83 (s, 2H), 2.80-2.67 (m, 2H), 2.22 (td, J=2.4, 19.2 Hz, 2H), 1.27 (s, 6H), 1.08 (s, 9H).

### Step 4: tert-butyl((1-(iodomethyl)-3,3-dimethyl-2-oxabicyclo[2.1.1]hexan-4-yl)methoxy)diphenylsilane

To a solution of 2-[1-[[tert-butyl(diphenyl)silyl]oxymethyl]-3-methylene-cyclobutyl]propan-2-ol (680 mg, 1.72 mmol, 1 eq) in MTBE (5 mL) and H₂O (2.5 mL) was added 12 (874.71 mg, 3.45 mmol, 694.22 μL, 2 eq) and NaHCO₃ (289.52 mg, 3.45 mmol, 134.10 μL, 2 eq). The mixture was stirred at 25° C. for 1 h. TLC (SiO₂, PE:EtOAc=4:1) indicated reactant was consumed completely and one new spot was formed. The reaction mixture was concentrated under the reduced pressure to give the crude product. The reaction mixture was quenched with saturated Na₂SO₃ (10 mL), and then the resulting mixture was extracted with EtOAc (10 mL×2). The combined organic layers was dried over anhydrous Na₂SO₄, filtered and concentrated in vacuo to give compound tert-butyl-[[1-(iodomethyl)-3,3-dimethyl-2-oxabicyclo[2.1.1]hexan-4-yl]methoxy]-diphenyl-silane (800 mg, 1.54 mmol, 89.19% yield) as yellow oil.

¹H NMR (400 MHz, CHLOROFORM-d) δ=7.71-7.59 (m, 4H), 7.50-7.35 (m, 6H), 3.78 (s, 2H), 3.42 (s, 2H), 1.87 (dd, J=1.6, 4.8 Hz, 2H), 1.71 (dd, J=1.2, 4.8 Hz, 2H), 1.27 (s, 6H), 1.07 (s, 9H).

### Step 5: (4-(((tert-butyldiphenylsilyl)oxy)methyl)-3,3-dimethyl-2-oxabicyclo[2.1.1]hexan-1-yl)methyl acetate

To a solution of tert-butyl-[[1-(iodomethyl)-3,3-dimethyl-2-oxabicyclo[2.1.1]hexan-4-yl]methoxy]-diphenyl-silane (400 mg, 768.47 μmol, 1 eq) in DMF (4 mL) was added KOAc (113.13 mg, 1.15 mmol, 1.5 eq). The mixture was stirred at 80° C. for 1 h. TLC (SiO₂, PE:EtOAc=4:1) indicated reactant was consumed completely and one new spot was formed. The mixture was quenched with water (10 mL) and extracted with ethyl acetate (15 mL×3). The combined organic phase was washed with brine (20 mL), dried with anhydrous Na₂SO₄, filtered and concentrated in vacuum. The residue was purified by prep-TLC (SiO₂, PE:EtOAc=4:1, Rf=0.5) to give compound [4-[[tert-butyl(diphenyl)silyl]oxymethyl]-3,3-dimethyl-2-oxabicyclo[2.1.1]hexan-1-yl]methyl acetate (250 mg, 552.30 μmol, 71.87% yield) as yellow oil.

¹H NMR (400 MHz, CHLOROFORM-d) δ=7.66 (dd, J=1.2, 7.6 Hz, 4H), 7.48-7.35 (m, 6H), 4.27 (s, 2H), 3.78 (s, 2H), 2.10 (s, 3H), 1.86 (dd, J=1.6, 4.8 Hz, 2H), 1.73-1.66 (m, 2H), 1.28 (s, 6H), 1.07 (s, 9H).

### Step 6: (4-(((tert-butyldiphenylsilyl)oxy)methyl)-3,3-dimethyl-2-oxabicyclo[2.1.1]hexan-1-yl)methanol

To a solution of [4-[[tert-butyl(diphenyl)silyl]oxymethyl]-3,3-dimethyl-2-oxabicyclo[2.1.1]hexan-1-yl]methyl acetate (250 mg, 552.30 μmol, 1 eq) in MeOH (2 mL) was added LiOH·H₂O (6.95 mg, 165.69 μmol, 0.3 eq). The mixture was stirred at 25° C. for 1 h. TLC (SiO₂, PE:EtOAc=3:1) indicated reactant was consumed completely and one new spot was formed. The pH was adjusted to around 7 by aqueous. HCl, and then extracted with ethyl acetate (10 mL×2). The combined organic layers were washed with brine (20 mL), dried over Na₂SO₄, filtered and concentrated under reduced pressure to give compound [4-[[tert-butyl(diphenyl)silyl]oxymethyl]-3,3-dimethyl-2-oxabicyclo[2.1.1]hexan-1-yl]methanol (262 mg, crude) as colorless oil.

¹H NMR (400 MHz, CHLOROFORM-d) δ=7.69-7.61 (m, 4H), 7.50-7.35 (m, 6H), 3.78 (d, J=3.2 Hz, 4H), 1.87 (dd, J=1.6, 4.8 Hz, 2H), 1.66 (dd, J=1.2, 4.8 Hz, 2H), 1.27 (s, 6H), 1.07 (s, 9H).

### Step 7: 4-(((tert-butyldiphenylsilyl)oxy)methyl)-3,3-dimethyl-2-oxabicyclo[2.1.1]hexane-1-carboxylic acid

To a solution of [4-[[tert-butyl(diphenyl)silyl]oxymethyl]-3,3-dimethyl-2-oxabicyclo[2.1.1]hexan-1-yl]methanol (450 mg, 1.10 mmol, 1 eq) in CH₃CN (2.5 mL) and H₂O (2.5 mL) was added RuCl₃ (22.73 mg, 109.59 μmol, 7.31 μL, 0.1 eq) and sodium;periodate (937.62 mg, 4.38 mmol, 242.91 μL, 4 eq). The mixture was stirred at 25° C. for 1 h. LCMS showed starting material was consumed and the desired mass was detected. The reaction mixture was extracted with ethyl acetate (20 mL×2). The combined organic phase was washed with brine (30 mL), dried with anhydrous Na₂SO₄, filtered and concentrated under reduced pressure to give compound 4-[[tert-butyl(diphenyl)silyl]oxymethyl]-3,3-dimethyl-2-oxabicyclo[2.1.1]hexane-1-carboxylic acid (460 mg, crude) as gray oil.

LC-MS [ESI, M+23]: 447.3.

¹H NMR (400 MHz, CHLOROFORM-d) δ=7.71-7.62 (m, 4H), 7.43 (qd, J=6.8, 13.6 Hz, 6H), 3.77 (s, 2H), 2.19 (br d, J=4.4 Hz, 2H), 2.09 (br d, J=4.4 Hz, 2H), 1.31 (s, 6H), 1.07 (s, 9H).

### Step 8: 6-bromo-2-(4-(((tert-butyldiphenylsilyl)oxy)methyl)-3,3-dimethyl-2-oxabicyclo[2.1.1]hexan-1-yl)-8-methyl-[1,2,4]triazolo[1,5-a]pyridine

To a solution of 4-[[tert-butyl(diphenyl)silyl]oxymethyl]-3,3-dimethyl-2-oxabicyclo[2.1.1]hexane-1-carboxylic acid (200 mg, 471.03 μmol, 1 eq) in DMF (2 mL) was added 5-bromo-3-methyl-pyridin-1-ium-1,2-diamine;2,4,6-trimethylbenzenesulfonate (189.50 mg, 471.03 μmol, 1 eq), HATU (268.65 mg, 706.54 μmol, 1.5 eq) and DIEA (182.63 mg, 1.41 mmol, 246.13 μL, 3 eq). The mixture was stirred at 25° C. for 1 h. LCMS showed starting material was consumed and the desired mass was detected. The reaction mixture was extracted with ethyl acetate (15 mL×2). The combined organic phase was washed with brine (30 mL), dried with anhydrous Na₂SO₄, filtered and concentrated in vacuum. The residue was purified by prep-TLC (SiO₂, PE:EtOAc=1:1, Rf=0.50) to give compound [1-(6-bromo-8-methyl-[1,2,4]triazolo[1,5-a]pyridin-2-yl)-3,3-dimethyl-2-oxabicyclo[2.1.1]hexan-4-yl]methoxy-tert-butyl-diphenyl-silane (100 mg, 160.84 μmol, 34.15% yield, 94.997% purity) as gray oil.

LC-MS [ESI, M+1]: 592.3.

¹H NMR (400 MHz, CHLOROFORM-d) δ=8.48 (d, J=0.8 Hz, 1H), 7.61 (dd, J=1.6, 7.6 Hz, 4H), 7.38-7.29 (m, 7H), 3.81 (s, 2H), 2.58 (s, 3H), 2.28-2.23 (m, 4H), 1.35 (s, 6H), 1.00 (s, 9H).

### Step 9: (R)-2-(4-(((tert-butyldiphenylsilyl)oxy)methyl)-3,3-dimethyl-2-oxabicyclo[2.1.1]hexan-1-yl)-8-methyl-6-(3-methyl-3-phenylpyrrolidin-1-yl)-[1,2,4]triazolo[1,5-a]pyridine

A mixture of [1-(6-bromo-8-methyl-[1,2,4]triazolo[1,5-a]pyridin-2-yl)-3,3-dimethyl-2-oxabicyclo[2.1.1]hexan-4-yl]methoxy-tert-butyl-diphenyl-silane (100 mg, 169.31 μmol, 1 eq), (3R)-3-methyl-3-phenyl-pyrrolidine (27.30 mg, 169.31 μmol, 1 eq), 1,3-bis[2,6-bis(1-propylbutyl)phenyl]-4,5-dichloro-2H-imidazol-1-ium-2-ide;3-chloropyridine;dichloropalladium (8.24 mg, 8.47 μmol, 0.05 eq) and Cs₂CO₃ (137.91 mg, 423.28 μmol, 2.5 eq) in dioxane (1 mL) was degassed and purged with N₂ for 3 times, and then the mixture was stirred at 100° C. for 12 h under N₂ atmosphere. LCMS showed starting material was consumed and the desired mass was detected. The reaction mixture was filtered and the filtrate was concentrated under the reduced pressure to give the product. The residue was purified by prep-TLC (SiO₂, PE:EtOAc=1:2, Rf=0.70) to give compound tert-butyl-[[3,3-dimethyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methoxy]-diphenyl-silane (70 mg, 102.82 μmol, 60.73% yield, 98.557% purity) as gray oil.

LC-MS [ESI, M+1]: 671.5.

¹H NMR (400 MHz, CHLOROFORM-d) δ=7.74-7.63 (m, 5H), 7.48-7.27 (m, 10H), 7.26-7.22 (m, 1H), 6.94 (br s, 1H), 3.89 (s, 2H), 3.58 (d, J=8.4 Hz, 1H), 3.54-3.42 (m, 2H), 3.37 (dt, J=3.6, 8.4 Hz, 1H), 2.71-2.59 (m, 3H), 2.48-2.18 (m, 6H), 1.46 (s, 3H), 1.43 (s, 6H), 1.08 (s, 9H).

### Step 10: (R)-(3,3-dimethyl-1-(8-methyl-6-(3-methyl-3-phenylpyrrolidin-1-yl)-[1,2,4]triazolo[1,5-a]pyridin-2-yl)-2-oxabicyclo[2.1.1]hexan-4-yl)methanol

To a solution of tert-butyl-[[3,3-dimethyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methoxy]-diphenyl-silane (35 mg, 52.16 μmol, 1 eq) in THF (0.5 mL) was added TBAF (1 M, 78.24 μL, 1.5 eq). The mixture was stirred at 25° C. for 0.5 h. LCMS showed starting material was consumed and the desired mass was detected. The reaction mixture was concentrated under the reduced pressure to give the crude product. The residue was purified by prep-HPLC (FA condition column: Welch Xtimate C18 150*25 mm*5 um; mobile phase: [water(FA)—ACN]; gradient:34%-64% B over 10 min) to give compound [3,3-dimethyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanol (10.66 mg, 24.18 μmol, 46.36% yield, 98.131% purity) as white solid.

LC-MS [ESI, M+1]:433.3.

¹H NMR (400 MHz, CHLOROFORM-d) δ=7.68 (br s, 1H), 7.42-7.27 (m, 5H), 6.95 (br s, 1H), 3.93 (d, J=3.2 Hz, 2H), 3.59 (br dd, J=2.8, 8.8 Hz, 1H), 3.55-3.45 (m, 2H), 3.42-3.35 (m, 1H), 2.67 (br d, J=4.0 Hz, 3H), 2.37 (br d, J=3.2 Hz, 5H), 2.31-2.20 (m, 2H), 1.49 (br d, J=3.5 Hz, 9H).

### Example 53

### N-[1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]propanamide (Compound 475)

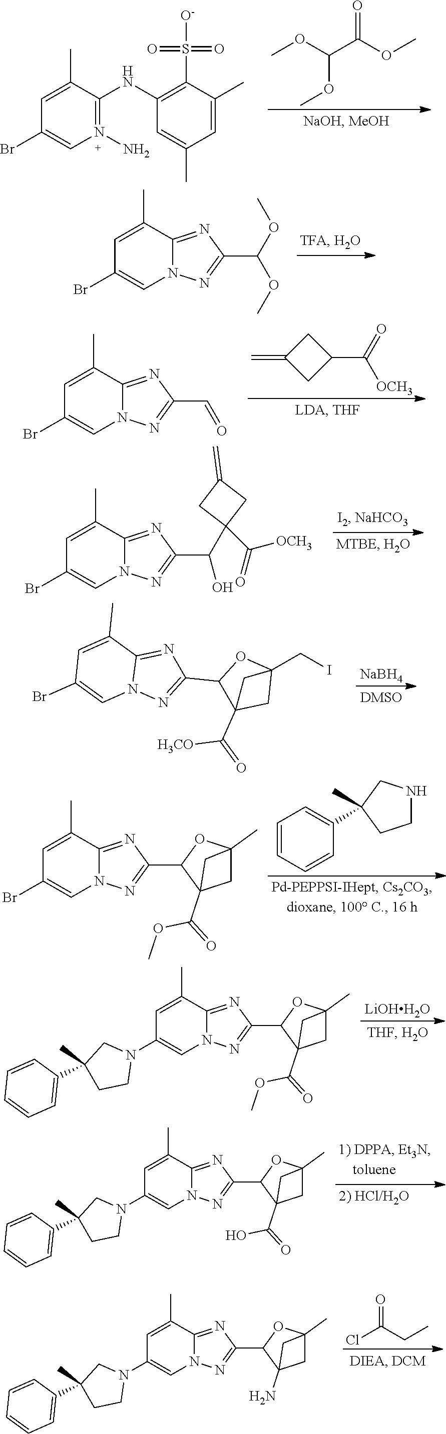

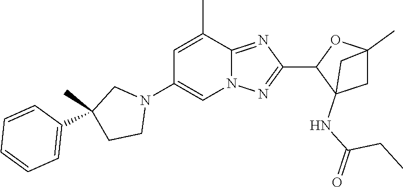 [SMILES: CCC(=O)NC12CC(C)(C1)OC2c1nc2c(C)cc(N3CC[C@](C)(c4ccccc4)C3)cn2n1]

### Step 1: 6-bromo-2-(dimethoxymethyl)-8-methyl-[1,2,4]triazolo[1,5-a]pyridine

To a solution of 5-bromo-3-methyl-pyridin-1-ium-1,2-diamine;2,4,6-trimethylbenzenesulfonate (10 g, 24.86 mmol, 1 eq) and methyl 2,2-dimethoxyacetate (6.67 g, 49.71 mmol, 6.10 mL, 2 eq) in MeOH (100 mL) was added NaOH (1.49 g, 37.29 mmol, 1.5 eq). The mixture was stirred at 70° C. for 12 h. LCMS showed starting material was consumed. Several new peaks were shown on LCMS and 52.97% of desired compound was detected. The reaction mixture was filtered and concentrated under reduced pressure to give a residue. The residue was purified by column chromatography (SiO₂, PE:EtOAc=1:1, Rf=0.4) to give compound 6-bromo-2-(dimethoxymethyl)-8-methyl-[1,2,4]triazolo[1,5-a]pyridine (4.98 g, 69.52% yield) as a yellow solid.

LC-MS [ESI, M-32+1]: 254.0.

¹H NMR (400 MHz, CHLOROFORM-d) δ=8.56 (s, 1H), 7.39 (s, 1H), 5.72 (s, 1H), 3.48 (s, 6H), 2.64 (s, 3H)

### Step 2: 6-bromo-8-methyl-[1,2,4]triazolo[1,5-a]pyridine-2-carbaldehyde

To a solution of 6-bromo-2-(dimethoxymethyl)-8-methyl-[1,2,4]triazolo[1,5-a]pyridine (6.2 g, 21.67 mmol, 1 eq) in H₂O (5 mL) was added dropwise TFA (20 mL) dropwise at 0° C. After addition, the mixture was stirred at 80° C. for 1 h. LCMS showed starting material consumed. Several new peaks were shown on LCMS and 56.26% of desired compound was detected. The reaction mixture was concentrated under reduced pressure to give a residue. The residue was diluted with NaHCO₃ (50 mL) and extracted with EtOAc (80 mL×3). The combined organic layers were dried over Na₂SO₄, filtered and concentrated under reduced pressure to give the crude 6-bromo-8-methyl-[1,2,4]triazolo[1,5-a]pyridine-2-carbaldehyde (4.73 g, 62.33% yield) as yellow solid.

LC-MS [ESI, M+1]: 240.1.

¹H NMR (400 MHz, CHLOROFORM-d) δ=10.22 (s, 1H), 8.67 (d, J=0.4 Hz, 1H), 7.51 (s, 1H), 2.71 (s, 3H)

### Step 3: methyl 1-[(6-bromo-8-methyl-[1,2,4]triazolo[1,5-a]pyridin-2-yl)-hydroxy-methyl]-3-methylene-cyclobutanecarboxylate

To a solution of methyl 3-methylenecyclobutanecarboxylate (2.49 g, 19.71 mmol, 1 eq) in THF (200 mL) was added LDA (2 M, 24.63 mL, 2.5 eq) at −78° C. under N₂ atmosphere, after 1 h, 6-bromo-8-methyl-[1,2,4]triazolo[1,5-a]pyridine-2-carbaldehyde (4.73 g, 19.71 mmol, 1 eq) was added in reaction mixture, the resulting mixture was stirred for another 1 h at −78° C. LCMS showed starting material consumed. Several new peaks were shown on LCMS and 38.77% of desired compound was detected. The reaction mixture was quenched by addition NH₄Cl (30 mL) at −20° C., and then diluted with H₂O (50 mL) and extracted with EtOAc (60 mL×3). The combined organic layers were washed with brine (50 mL×3), dried over Na₂SO₄, filtered and concentrated under reduced pressure to give a residue. The residue was purified by flash silica gel chromatography (SiO₂, PE:EtOAc=1:1, Rf=0.3) to give the compound methyl 1-[(6-bromo-8-methyl-[1,2,4]triazolo[1,5-a]pyridin-2-yl)-hydroxy-methyl]-3-methylene-cyclobutanecarboxylate (1.41 g, 17.63% yield) as yellow solid.

LC-MS [ESI, M+1]: 366.0.

¹H NMR (400 MHz, CHLOROFORM-d) δ=8.62-8.46 (m, 1H), 7.38 (s, 1H), 5.19 (d, J=8.8 Hz, 1H), 4.85 (t, J=2.4 Hz, 2H), 3.88 (br d, J=9.2 Hz, 1H), 3.77 (s, 3H), 3.25-3.13 (m, 2H), 3.09-2.95 (m, 2H), 2.60 (s, 3H)

### Step 4: methyl 3-(6-bromo-8-methyl-[1,2,4]triazolo[1,5-a]pyridin-2-yl)-1-(iodomethyl)-2-oxabicyclo[2.1.1]hexane-4-carboxylate

A mixture of methyl 1-[(6-bromo-8-methyl-[1,2,4]triazolo[1,5-a]pyridin-2-yl)-hydroxy-methyl]-3-methylene-cyclobutanecarboxylate (1.41 g, 3.37 mmol, 1 eq) in H₂O (4 mL) and MTBE (12 mL) was added NaHCO₃ (566.74 mg, 6.75 mmol, 262.50 μL, 2 eq) and 12 (1.71 g, 6.75 mmol, 1.36 mL, 2 eq), and then the mixture was stirred at 25° C. for 1 h. LCMS showed starting material consumed. Several new peaks were shown on LCMS and 53.99% of desired compound was detected. The reaction mixture was quenched by addition Na₂SO₃ (20 mL) at 0° C., and then diluted with H₂O (30 mL) and extracted with EtOAc (40 mL×3). The combined organic layers were washed with brine (30 mL×3), dried over Na₂SO₄, filtered and concentrated under reduced pressure to give a residue. The residue was purified by flash silica gel chromatography (SiO₂, PE:EtOAc=1:1, Rf=0.5) to give compound methyl 3-(6-bromo-8-methyl-[1,2,4]triazolo[1,5-a]pyridin-2-yl)-1-(iodomethyl)-2-oxabicyclo[2.1.1]hexane-4-carboxylate (1.44 g, 81.73% yield) as yellow oil.

LC-MS [ESI, M+1]: 491.9.

¹H NMR (400 MHz, CHLOROFORM-d) δ=8.55 (s, 1H), 7.37 (s, 1H), 5.51 (s, 1H), 3.71 (s, 3H), 3.61-3.56 (m, 1H), 3.54-3.49 (m, 1H), 2.72-2.65 (m, 1H), 2.61 (s, 3H), 2.36-2.27 (m, 2H), 2.25 (d, J=8.0 Hz, 1H)

### Step 5: methyl 3-(6-bromo-8-methyl-[1,2,4]triazolo[1,5-a]pyridin-2-yl)-1-methyl-2-oxabicyclo[2.1.1]hexane-4-carboxylate

To a solution of methyl 3-(6-bromo-8-methyl-[1,2,4]triazolo[1,5-a]pyridin-2-yl)-1-(iodomethyl)-2-oxabicyclo[2.1.1]hexane-4-carboxylate (600 mg, 1.22 mmol, 1 eq) in DMSO (20 mL) was added NaBH₄ (184.51 mg, 4.88 mmol, 4 eq) at 0° C. under N₂ atmosphere. The suspension was degassed and purged with N₂ for 3 times. The mixture was stirred under N₂ at 25° C. for 2 h. LCMS showed starting material consumed. Several new peaks were shown on LCMS and 73.34% of desired compound was detected. The reaction mixture was quenched by addition NH₄Cl (8 mL) at 0° C., and then was diluted with H₂O (20 mL) and extracted with EtOAc (25 mL×3). The combined organic layers were washed with brine (15 mL×5), dried over Na₂SO₄, filtered and concentrated under reduced pressure to give a crude methyl 3-(6-bromo-8-methyl-[1,2,4]triazolo[1,5-a]pyridin-2-yl)-1-methyl-2-oxabicyclo[2.1.1]hexane-4-carboxylate (433 mg, 85.66% yield) as a white oil.

LC-MS [ESI, M+1]: 366.0.

### Step 6: methyl 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate

To a solution of methyl 3-(6-bromo-8-methyl-[1,2,4]triazolo[1,5-a]pyridin-2-yl)-1-methyl-2-oxabicyclo[2.1.1]hexane-4-carboxylate (504 mg, 1.22 mmol, 1 eq) in dioxane (0.5 mL) was added (3R)-3-methyl-3-phenyl-pyrrolidine (196.02 mg, 1.22 mmol, 1 eq), Cs₂CO₃ (990.21 mg, 3.04 mmol, 2.5 eq) and 1,3-bis[2,6-bis(1-propylbutyl)phenyl]-4,5-dichloro-2H-imidazol-1-ium-2-ide; 3-chloropyridine;dichloropalladium (59.13 mg, 60.78 μmol, 0.05 eq). The resulting mixture was stirred at 100° C. for 16 h. LCMS showed starting material consumed. Several new peaks were shown on LCMS and 71.2% of desired compound was detected. The reaction mixture was filtered and then was diluted with H₂O (20 mL) and extracted with EtOAc (30 mL×3). The combined organic layers were washed with brine (20 mL×3), dried over Na₂SO₄, filtered and concentrated under reduced pressure to give a residue. The residue was purified by flash silica gel chromatography (SiO₂, PE:EtOAc=1:1, Rf=0.4) to give compound methyl 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (372 mg, 59.78% yield) as white oil.

LC-MS [ESI, M+1]: 447.2.

¹H NMR (400 MHz, CHLOROFORM-d) δ=7.60 (d, J=1.6 Hz, 1H), 7.36-7.29 (m, 2H), 7.27 (s, 2H), 7.22-7.17 (m, 1H), 6.86 (s, 1H), 5.45-5.37 (m, 1H), 3.68-3.62 (m, 3H), 3.53 (d, J=8.8 Hz, 1H), 3.49-3.38 (m, 2H), 3.36-3.29 (m, 1H), 2.61-2.56 (m, 1H), 2.55 (s, 3H), 2.31 (td, J=8.4, 12.1 Hz, 1H), 2.24-2.15 (m, 2H), 2.15-2.11 (m, 1H), 2.04-1.99 (m, 1H), 1.54-1.50 (m, 3H), 1.41 (s, 3H)

### Step 7: 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (Compound 474)

To a solution of methyl 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (372 mg, 833.07 μmol, 1 eq) in THF (3 mL) and H₂O (1 mL) was added LiOH·H₂O (69.92 mg, 1.67 mmol, 2 eq). The mixture was stirred at 25° C. for 2 h. LCMS showed starting material consumed. Several new peaks were shown on LCMS and 92.81% of desired compound was detected. The reaction mixture was filtered and then was concentrated under reduced pressure to give a crude 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (256 mg, 69.92% yield) as white oil.

LC-MS [ESI, M+1]: 433.2.

¹H NMR (400 MHz, DMSO-d₆) δ=7.88 (d, J=1.6 Hz, 1H), 7.42-7.32 (m, 4H), 7.27-7.19 (m, 2H), 5.12 (s, 1H), 3.60-3.55 (m, 1H), 3.53-3.42 (m, 3H), 3.41-3.26 (m, 3H), 2.64 (dd, J=7.2, 10.0 Hz, 1H), 2.25 (t, J=6.8 Hz, 2H), 2.12 (d, J=6.8 Hz, 1H), 2.04-1.94 (m, 2H), 1.40 (s, 3H), 1.35 (s, 3H)

### Step 8: 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine

To a solution of 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (50 mg, 115.60 μmol, 1 eq) in toluene (1 mL) was added DPPA (35.00 mg, 127.16 μmol, 27.45 μL, 1.1 eq) and Et₃N (12.87 mg, 127.16 μmol, 17.70 μL, 1.1 eq) under N₂ atmosphere. After addition, the mixture was stirred at 80° C. for 3 h and then HCl (2 M, 5 mL, 86.50 eq) was added dropwise at 25° C. The resulting mixture was stirred at 25° C. for 1 h. LCMS showed starting material consumed. Several new peaks were shown on LCMS and 68.06% of desired compound was detected. The reaction mixture was diluted with H₂O (20 mL) and extracted with EtOAc (25 mL×3). The combined aqueous phase was freeze-dried to give a residue. The residue was purified by prep-HPLC (FA condition) to give compound 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (25 mg, 35.50% yield) as white solid.

LC-MS [ESI, M+1]: 404.2.

### Step 9: N-[1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]propanamide

To a solution of 1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-amine (20 mg, 49.56 μmol, 1 eq) in DCM (0.5 mL) was added propanoyl chloride (6.88 mg, 74.35 μmol, 6.88 μL, 1.5 eq) and DIEA (19.22 mg, 148.69 μmol, 25.90 μL, 3 eq) slowly at 0° C. The resulting mixture was stirred at 25° C. for 1 h. LCMS showed starting material was consumed. Several new peaks were shown on LCMS and 59.71% of desired compound was detected. The reaction mixture was concentrated under reduced pressure to give a residue. The residue was purified by prep-HPLC (FA condition, column: Phenomenex luna C18 150×25 mm×10 um; mobile phase: [water (FA)—ACN]; gradient: 44%-74% B over 10 min) to give compound N-[1-methyl-3-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]propanamide (0.88 mg, 3.86% yield) as white solid.

LC-MS [ESI, M+1]: 460.4.

¹H NMR (400 MHz, CHLOROFORM-d) δ=7.71 (s, 1H), 7.41-7.34 (m, 2H), 7.33-7.28 (m, 3H), 7.24 (br d, J=4.4 Hz, 1H), 6.96 (s, 1H), 5.11 (s, 1H), 3.59 (d, J=8.0 Hz, 1H), 3.55-3.43 (m, 2H), 3.43-3.31 (m, 1H), 2.91 (d, J=7.2 Hz, 1H), 2.60 (s, 3H), 2.42-2.31 (m, 2H), 2.29-2.20 (m, 3H), 2.09 (dd, J=7.2, 10.3 Hz, 1H), 1.97 (dd, J=7.6, 10.3 Hz, 1H), 1.60 (s, 3H), 1.46 (s, 3H), 1.16 (t, J=7.6 Hz, 3H)

| Compound | | |
|---|---|---|
| Number | Synthesis | LC/MS |
| 474 | Example 53 | m/z 432.52 |
| | | [M + H]⁺ |
| 475 | Similar to | m/z 459.59 |
| | Example 53 | [M + H]⁺ |
| 476 | Similar to | m/z 431.54 |
| | Example 53 | [M + H]⁺ |

### Example 54

### Compound 1: 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazole-3-carboxamide (Compound 477)

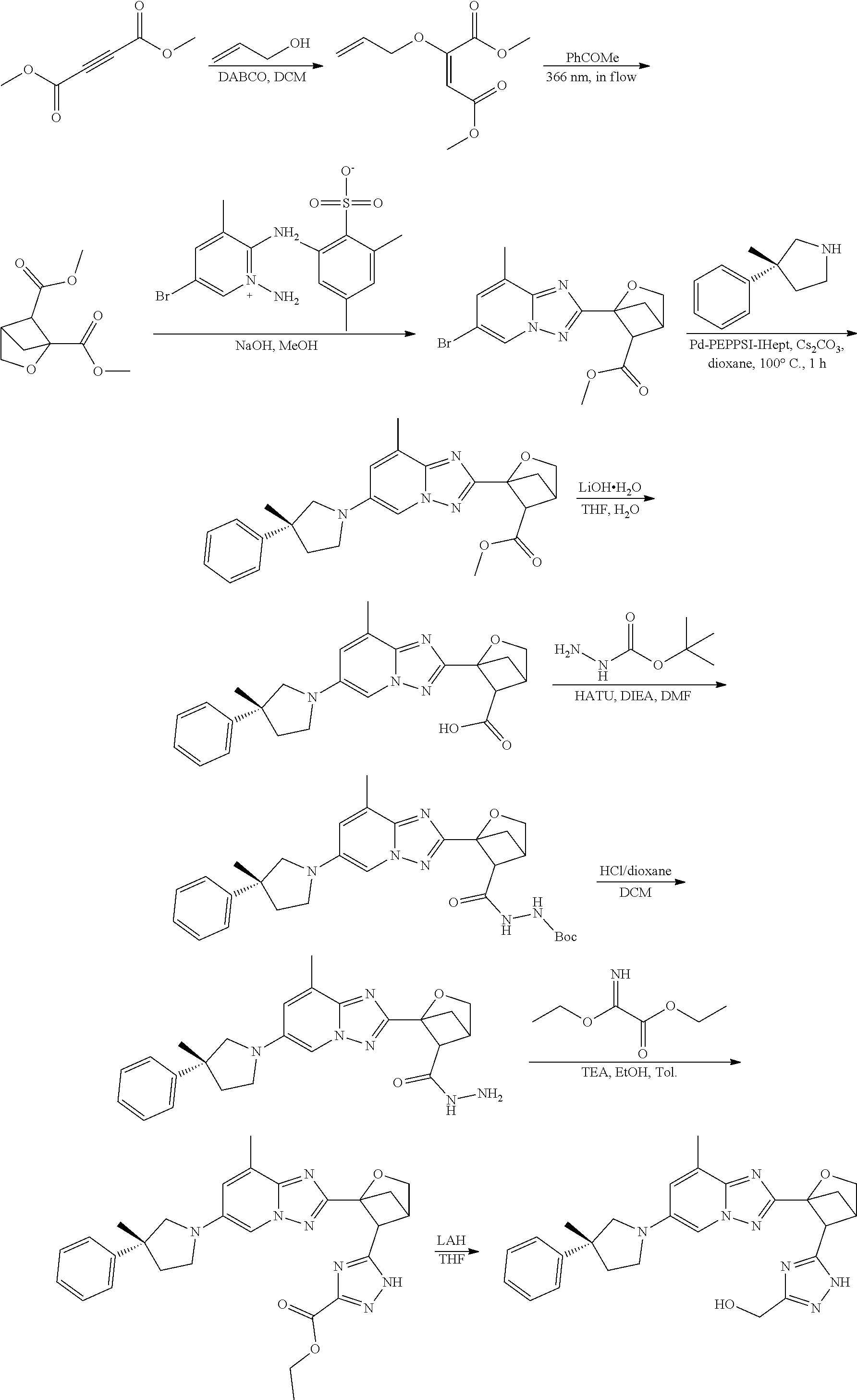

### Step 1: dimethyl (E)-2-allyloxybut-2-enedioate

To a solution of dimethyl but-2-ynedioate (50 g, 351.84 mmol, 1 eq) in DCM (200 mL) was added prop-2-en-1-ol (21.46 g, 369.43 mmol, 25.12 mL, 1.05 eq). Then the solution was dropwise added DABCO (3.95 g, 35.18 mmol, 3.87 mL, 0.1 eq) in DCM (30 mL). The mixture was stirred at 25° C. for 3 h. TLC (PE:EtOAc=5:1) showed starting material was not consumed completely and many new spots formed. The reaction was concentrated under reduced pressure to give a residue. The residue was purified by column chromatography (SiO₂, PE:EtOAc=5:1, Rf=0.45) to give compound dimethyl (E)-2-allyloxybut-2-enedioate (130 g, 46% yield) as a colourless liquid.

¹H NMR (400 MHz, CHLOROFORM-d) δ=5.97-5.86 (m, 1H), 5.41-5.26 (m, 2H), 5.17 (s, 1H), 4.39 (d, J=5.2 Hz, 2H), 3.85 (s, 3H), 3.66 (s, 3H).

### Step 2: dimethyl 2-oxabicyclo[2.1.1]hexane-1,5-dicarboxylate

To a solution of dimethyl (E)-2-allyloxybut-2-enedioate (120 g, 599.44 mmol, 1 eq) in ACN (1000 mL) was added diphenylmethanone (10.92 g, 59.94 mmol, 0.1 eq). The mixture was stirred at 25° C. (366 nm, in flow) for 1 h. TLC (PE:EtOAc=5:1) showed starting material was not consumed completely and three new spots formed. The reaction mixture was concentrated in vacuum to give compound dimethyl 2-oxabicyclo[2.1.1]hexane-1,5-dicarboxylate (120 g, crude).as a yellow liquid.

### Step 3: methyl 1-(6-bromo-8-methyl-[1,2,4]triazolo[1,5-a]pyridin-2-yl)-2-oxabicyclo[2.1.1]hexane-5-carboxylate

To a solution of dimethyl 2-oxabicyclo[2.1.1]hexane-1,5-dicarboxylate (37.82 g, 188.91 mmol, 2 eq) and 5-bromo-3-methyl-pyridin-1-ium-1,2-diamine;2,4,6-trimethylbenzenesulfonate (38 g, 94.46 mmol, 1 eq) in MeOH (500 mL) was added NaOH (5.67 g, 141.68 mmol, 1.5 eq). The mixture was stirred at 70° C. for 1 h. LCMS showed starting material was consumed completely and the desired compound was detected. The reaction was concentrated under reduced pressure to give a residue. The residue was purified by column chromatography (SiO₂, PE:EtOAc=1:1, Rf=0.19) to give compound methyl 1-(6-bromo-8-methyl-[1,2,4]triazolo[1,5-a]pyridin-2-yl)-2-oxabicyclo[2.1.1]hexane-5-carboxylate (8 g, 24% yield) as a yellow solid.

LC-MS [ESI, M+1]: 352.5.

¹H NMR (400 MHz, DMSO-d6) δ=9.18 (d, J=0.4 Hz, 1H), 7.68 (s, 1H), 4.02-3.98 (m, 1H), 3.86 (d, J=6.4 Hz, 1H), 3.50 (s, 3H), 3.32 (s, 1H), 3.23 (t, J=2.8 Hz, 1H), 2.53 (s, 3H), 2.19 (dd, J=7.2, 2.8 Hz, 1H), 1.70 (d, J=7.2 Hz, 1H).

### Step 4: methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

To a solution of methyl 1-(6-bromo-8-methyl-[1,2,4]triazolo[1,5-a]pyridin-2-yl)-2-oxabicyclo[2.1.1]hexane-5-carboxylate (5 g, 14.20 mmol, 1 eq) and (3R)-3-methyl-3-phenyl-pyrrolidine (2.29 g, 14.20 mmol, 1 eq) in dioxane (80 mL) was added Pd-PEPPSI-IHeptCl (690.53 mg, 709.86 μmol, 0.05 eq) and Cs₂CO₃ (13.88 g, 42.59 mmol, 3 eq). The mixture was stirred at 100° C. for 1 h under N₂. LCMS showed starting material was consumed completely and the desired compound was detected. The reaction was concentrated under reduced pressure to give a residue. The residue was purified by column chromatography (SiO₂, PE:EtOAc=1:1, Rf=0.44) to give compound methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (4.7 g, 76% yield) as a brown solid.

LC-MS [ESI, M+1]: 433.3.

¹H NMR (400 MHz, CHLOROFORM-d) δ=7.66 (s, 1H), 7.40-7.33 (m, 2H), 7.33-7.28 (m, 2H), 7.27-7.22 (m, 1H), 6.96-6.91 (m, 1H), 4.27 (d, J=6.4 Hz, 1H), 4.02 (d, J=6.4 Hz, 1H), 3.63-3.55 (m, 4H), 3.54-3.44 (m, 2H), 3.38 (dt, J=8.8, 3.6 Hz, 1H), 3.34-3.26 (m, 2H), 2.67-2.60 (m, 3H), 2.40-2.32 (m, 1H), 2.30-2.20 (m, 2H), 1.90 (d, J=8.0 Hz, 1H), 1.46 (s, 3H).

### Step 5: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid

To a solution of methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (1.50 g, 3.47 mmol, 1 eq) in THF (15 mL) was added LiOH·H₂O (436.56 mg, 10.40 mmol, 3 eq) and H₂O (2 mL). The mixture was stirred at 25° C. for 1 h. LCMS showed starting material was consumed completely and the desired compound was detected. The reaction was added HCl (2 M) until pH around 3 and then extracted with EtOAc (30 mL×5). The combined organic phase was washed with brine (20 mL×3), dried over anhydrous Na₂SO₄, filtered and concentrated in vacuum to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.4 g, 96% yield) as a yellow oil.

LC-MS [ESI, M+1]: 419.2.

### Step 6: tert-butyl N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]carbamate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (900.00 mg, 2.15 mmol, 1 eq) and tert-butyl N-aminocarbamate (341.07 mg, 2.58 mmol, 1.2 eq) in DMF (12 mL) was added HATU (1.23 g, 3.23 mmol, 1.5 eq) and DIEA (833.85 mg, 6.45 mmol, 1.12 mL, 3 eq). The mixture was stirred at 25° C. for 1 h. LCMS showed starting material was consumed completely and the desired compound was detected. The reaction was added H₂O (10 mL) and then extracted with EtOAc (30 mL×3). The combined organic phase was washed with brine (20 mL×3), dried over anhydrous Na₂SO₄, filtered and concentrated in vacuum. The residue was purified by column chromatography (SiO₂, PE:EtOAc=0:1, Rf=0.5) to give compound tert-butyl N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]carbamate (950 mg, 83% yield) as a yellow oil.

LC-MS [ESI, M+1]: 533.3

¹H NMR (400 MHz, CHLOROFORM-d) δ=10.73 (d, J=7.2 Hz, 1H), 7.63 (s, 1H), 7.40-7.34 (m, 2H), 7.34-7.29 (m, 2H), 7.25 (s, 1H), 6.98 (s, 1H), 6.68-6.35 (m, 1H), 4.17 (d, J=6.4 Hz, 1H), 4.01 (d, J=6.4 Hz, 1H), 3.60 (d, J=8.8 Hz, 1H), 3.55-3.46 (m, 2H), 3.45-3.35 (m, 2H), 3.10 (s, 1H), 2.62 (s, 3H), 2.42-2.33 (m, 1H), 2.29-2.22 (m, 1H), 2.18 (dd, J=7.6, 2.8 Hz, 1H), 2.08 (s, 1H), 1.50-1.46 (m, 12H).

### Step 7: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbohydrazide

To a solution of tert-butyl N-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]carbamate (950 mg, 1.78 mmol, 1 eq) in DCM (6 mL) was added HCl/dioxane (2 M, 3.39 mL, 3.80 eq). The mixture was stirred at 25° C. for 0.5 h. LCMS showed starting material was consumed completely and the desired compound was detected. The reaction mixture was concentrated in vacuum. The reaction was added NaHCO₃ until pH around 7 and then extracted with DCM (15 mL×5). The combined organic phase was washed with brine (10 mL×3), dried over anhydrous Na₂SO₄, filtered and concentrated in vacuum to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbohydrazide (715 mg, 92% yield) as a yellow oil.

LC-MS [ESI, M+1]: 433.2.

### Step 8: ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazole-3-carboxylate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbohydrazide (600 mg, 1.39 mmol, 1 eq) and ethyl 2-ethoxy-2-imino-acetate (503.89 mg, 3.47 mmol, 2.50 eq) in EtOH (6 mL) was added TEA (210.56 mg, 2.08 mmol, 289.63 μL, 1.5 eq). The mixture was stirred at 80° C. for 1 h and then the solution was added toluene (5 mL). The mixture was stirred at 145° C. for 12 h. LCMS showed starting material was consumed completely and the desired compound was detected. The mixture was filtered and concentrated under pressure to give the product. The residue was purified by prep-TLC (SiO₂, DCM:MeOH=10:1, Rf=0.45) to give compound ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazole-3-carboxylate (80 mg, 11% yield) as a brown solid.

LC-MS [ESI, M+1]: 514.3.

¹H NMR (400 MHz, CHLOROFORM-d) δ=7.68 (s, 1H), 7.42-7.34 (m, 2H), 7.34-7.30 (m, 2H), 7.27-7.23 (m, 1H), 7.00 (s, 1H), 4.61-4.49 (m, 1H), 4.46-4.32 (m, 2H), 4.06 (d, J=9.2 Hz, 1H), 3.62 (dd, J=8.8, 3.6 Hz, 1H), 3.55-3.45 (m, 2H), 3.44-3.33 (m, 2H), 3.25 (s, 2H), 3.07 (d, J=12.4 Hz, 1H), 2.72 (dd, J=12.4, 5.6 Hz, 1H), 2.66 (s, 3H), 2.42-2.33 (m, 1H), 2.32-2.21 (m, 1H), 1.48 (s, 3H), 1.36 (t, J=7.2 Hz, 3H).

### Step 9: [5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]methanol

To a solution of ethyl 5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazole-3-carboxylate (30 mg, 58.41 μmol, 1 eq) in THF (1.5 mL) was added LAH (4.43 mg, 116.82 μmol, 46.73 μL, 2 eq) at 0° C. The mixture was stirred at 0° C. for 0.2 h. LCMS showed reactant was consumed completely and the desired mass was detected. After the reaction mixture was cooled to 0, the reaction mixture was quenched by addition of 0.1 mL of H₂O, followed by 0.1 mL of 15% aqueous NaOH and 0.3 mL of H₂O. After being stirred at room temperature for 0.1 h, the solid was removed by filtration. The filtrate was concentrated to dryness to give crude product. [Purification] The residue was purified by prep-HPLC (column: CD02-Waters Xbidge BEH C18 150*25*10 um; mobile phase: [water(NH₃H₂O)—ACN]; gradient:32%-52% B over 10 min) to give compound [5-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]methanol (3.56 mg, 13% yield) as a yellow solid.

LC-MS [ESI, M+1]: 472.3.

¹H NMR (400 MHz, CHLOROFORM-d) δ=7.70 (s, 1H), 7.41-7.34 (m, 2H), 7.34-7.29 (m, 2H), 7.27-7.22 (m, 1H), 7.00 (s, 1H), 4.62 (s, 2H), 4.56-4.46 (m, 1H), 4.03 (d, J=9.2 Hz, 1H), 3.61 (dd, J=8.8, 2.4 Hz, 1H), 3.56-3.44 (m, 2H), 3.43-3.29 (m, 2H), 3.23-3.11 (m, 2H), 3.04 (d, J=12.4 Hz, 1H), 2.73-2.61 (m, 4H), 2.44-2.31 (m, 2H), 2.30-2.21 (m, 1H), 1.47 (s, 3H).

| Compound | | |
|---|---|---|
| Number | Synthesis | LC/MS |
| 478 | Similar to | m/z 539.2 |
| | Example 54 | [M + H]⁺ |
| 479 | Similar to | m/z 458.2 |
| | Example 54 | [M + H]⁺ |
| 480 | Similar to | m/z 500.3 |
| | Example 54 | [M + H]⁺ |
| 481 | Similar to | m/z 486.2 |
| | Example 54 | [M + H]⁺ |
| 482 | Similar to | m/z 486.1 |
| | Example 54 | [M + H]⁺ |
| 483 | Similar to | m/z 471.2 |
| | Example 54 | [M + H]⁺ |
| 484 | Similar to | m/z 455.2 |
| | Example 54 | [M + H]⁺ |
| 485 | Similar to | m/z 472.1 |
| | Example 54 | [M + H]⁺ |

### Example 55

### 8-methyl-2-[5-[(5-methyl-1,2,4-triazol-1-yl)methyl]-2-oxabicyclo[2.1.1]hexan-1-yl]-6-[(2R)-2-phenylpropoxy]-[1,2,4]triazolo[1,5-a]pyridine

### 8-methyl-2-(5-((3-methyl-11H-1,2,4-triazol-1-yl)methyl)-2-oxabicyclo[2.1.1]hexan-1-yl)-6-((R)-2-phenylpropoxy)-[1,2,4]triazolo[1,5-a]pyridine (Compound 487)

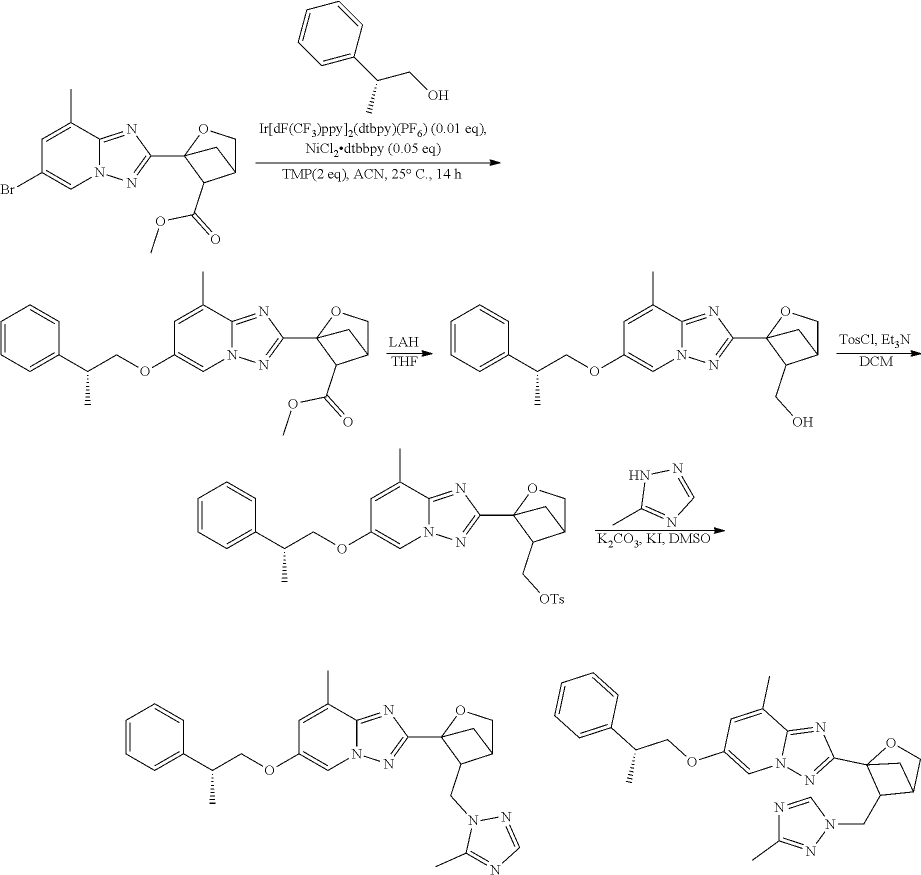

### Step 1: methyl 1-[8-methyl-6-[(2R)-2-phenylpropoxy]-[1,2,4]triazolo[1,5-a]pyridin-2-yl]-2-oxabicyclo[2.1.1]hexane-5-carboxylate

To a solution of methyl 1-(6-bromo-8-methyl-[1,2,4]triazolo[1,5-a]pyridin-2-yl)-2-oxabicyclo[2.1.1]hexane-5-carboxylate (1 g, 2.84 mmol, 1 eq) and (2R)-2-phenylpropan-1-ol (1.16 g, 8.52 mmol, 3 eq) in ACN (10 mL) was added Ir[dF(CF₃)ppy]₂(dtbpy)(PF₆) (31.86 mg, 28.39 μmol, 0.01 eq) NiCl₂·dtbbpy (56.50 mg, 141.97 μmol, 0.05 eq) and TMP (802.16 mg, 5.68 mmol, 964.14 μL, 2 eq). The mixture was stirred at 25° C. for 12 h. LCMS showed reactant was consumed completely and one main peak with desired mass was detected. The reaction mixture was filtered and the filtrate was concentrated under the reduced pressure to give the product. The residue was purified by column chromatography (SiO₂, PE:EtOAc=0:1, Rf=0.5) to give compound methyl 1-[8-methyl-6-[(2R)-2-phenylpropoxy]-[1,2,4]triazolo[1,5-a]pyridin-2-yl]-2-oxabicyclo[2.1.1]hexane-5-carboxylate (800 mg, 69% yield) as a yellow solid.

LC-MS [ESI, M+1]: 408.3.

¹H NMR (400 MHz, CHLOROFORM-d) δ=7.94 (s, 1H), 7.36-7.29 (m, 2H), 7.27 (s, 3H), 7.02 (s, 1H), 4.24 (d, J=6.4 Hz, 1H), 4.01 (s, 2H), 3.97-3.91 (m, 1H), 3.57 (s, 3H), 3.28 (s, 2H), 2.57 (s, 3H), 2.24 (d, J=7.2 Hz, 1H), 1.87 (d, J=7.6 Hz, 1H), 1.68 (s, 1H), 1.40 (d, J=7.2 Hz, 3H).

### Step 2: [1-[8-methyl-6-[(2R)-2-phenylpropoxy]-[1,2,4]triazolo[1,5-a]pyridin-2-yl]-2-oxabicyclo[2.1.1]hexan-5-yl]methanol

To a solution of LAH (2.5 M, 368.13 μL, 2.5 eq) in THF (3 mL) was added methyl 1-[8-methyl-6-[(2R)-2-phenylpropoxy]-[1,2,4]triazolo[1,5-a]pyridin-2-yl]-2-oxabicyclo[2.1.1]hexane-5-carboxylate (150 mg, 368.13 μmol, 1 eq) at 0° C. The mixture was stirred at 25° C. for 1 h. LCMS showed reactant was consumed completely and the desired mass was detected. After the reaction mixture was cooled to 0° C., the reaction mixture was quenched by addition of 0.2 mL of H₂O, followed by 0.2 mL of 15% aqueous NaOH and 0.6 mL of H₂O. After being stirred at room temperature for 0.5 h, the solid was removed by filtration. The filtrate was concentrated to dryness to give crude product to give compound [1-[8-methyl-6-[(2R)-2-phenylpropoxy]-[1,2,4]triazolo[1,5-a]pyridin-2-yl]-2-oxabicyclo[2.1.1]hexan-5-yl]methanol (130 mg, crude) as a yellow oil.

LC-MS [ESI, M+1]: 380.2.

### Step 3: [1-[8-methyl-6-[(2R)-2-phenylpropoxy]-[1,2,4]triazolo[1,5-a]pyridin-2-yl]-2-oxabicyclo[2.1.1]hexan-5-yl]methyl 4-methylbenzenesulfonate

To a solution of [1-[8-methyl-6-[(2R)-2-phenylpropoxy]-[1,2,4]triazolo[1,5-a]pyridin-2-yl]-2-oxabicyclo[2.1.1]hexan5-yl]methanol (130 mg, 342.60 μmol, 1 eq) in DCM (2.5 mL) was added TEA (104.00 mg, 1.03 mmol, 143.06 μL, 3 eq), DMAP (4.19 mg, 34.26 μmol, 0.1 eq) and TosCl (130.63 mg, 685.20 μmol, 2 eq) at 0° C. The mixture was stirred at 25° C. for 1 h. LCMS showed reactant was consumed completely and the desired compound was detected. The reaction was added H₂O (3 mL) and then extracted with DCM (15 mL×3). The combined organic phase was washed with brine (10 mL×3), dried over anhydrous Na₂SO₄, filtered and concentrated in vacuum. The residue was purified by prep-TLC (SiO₂, PE:EtOAc=1:2, Rf=0.5) and the eluted with EtOAc (60 mL) to give compound [1-[8-methyl-6-[(2R)-2-phenylpropoxy]-[1,2,4]triazolo[1,5-a]pyridin-2-yl]-2-oxabicyclo[2.1.1]hexan-5-yl]methyl 4-methylbenzenesulfonate (145 mg, 79% yield) as a yellow oil.

¹H NMR (400 MHz, CHLOROFORM-d) δ=7.87 (s, 1H), 7.73 (d, J=8.0 Hz, 2H), 7.36-7.30 (m, 2H), 7.26 (d, J=8.8 Hz, 5H), 7.01 (s, 1H), 4.11-4.07 (m, 1H), 4.06-3.99 (m, 2H), 3.96-3.90 (m, 2H), 3.74 (d, J=6.8 Hz, 1H), 3.25 (sxt, J=6.8 Hz, 1H), 3.03 (s, 1H), 2.76 (d, J=4.0 Hz, 1H), 2.53 (s, 3H), 2.40 (s, 3H), 2.15 (d, J=7.2 Hz, 1H), 1.92 (d, J=8.0 Hz, 1H), 1.41 (d, J=7.2 Hz, 3H).

### Step 4: 8-methyl-2-[5-[(5-methyl-1,2,4-triazol-1-yl)methyl]-2-oxabicyclo[2.1.1]hexan-1-yl]-6-[(2R)-2-phenylpropoxy]-[1,2,4]triazolo[1,5-a]pyridine (Compound 486)

To a solution of [1-[8-methyl-6-[(2R)-2-phenylpropoxy]-[1,2,4]triazolo[1,5-a]pyridin-2-yl]-2-oxabicyclo[2.1.1]hexan5-yl]methyl 4-methylbenzenesulfonate (50 mg, 93.70 μmol, 1 eq) and 5-methyl-1H-1,2,4-triazole (9.34 mg, 112.44 μmol, 1.2 eq) in DMSO (1 mL) was added K₂CO₃ (25.90 mg, 187.39 μmol, 2 eq) and KI (15.55 mg, 93.70 μmol, 1 eq). The mixture was stirred at 80° C. for 1 h. LCMS and HPLC showed reactant was consumed completely and the desired compound was detected. The reaction was added H₂O (3 mL) and then extracted with EtOAc (15 mL×3). The combined organic phase was washed with brine (10 mL×3), dried over anhydrous Na₂SO₄, filtered and concentrated in vacuum. The residue was purified by prep-HPLC (column: Phenomenex C18 150 mm*30 mm*5 m; mobile phase: [water(FA)—ACN]; gradient:32%-52% B over 8 min) to give compound 8-methyl-2-[5-[(5-methyl-1,2,4-triazol-1-yl)methyl]-2-oxabicyclo[2.1.1]hexan-1-yl]-6-[(2R)-2-phenylpropoxy]-[1,2,4]triazolo[1,5-a]pyridine (6.26 mg, 12% yield, TFA) as a colorless gum.

LC-MS [ESI, M+1]: 445.2.

¹H NMR (400 MHz, CHLOROFORM-d) δ=8.03 (d, J=3.2 Hz, 1H), 7.83 (s, 1H), 7.34-7.28 (m, 2H), 7.27 (s, 2H), 7.24-7.19 (m, 1H), 7.06 (s, 1H), 4.26 (dd, J=8.4, 14.4 Hz, 1H), 4.09-3.93 (m, 5H), 3.23 (sxt, J=6.8 Hz, 1H), 3.09 (s, 1H), 2.98 (s, 1H), 2.57 (d, J=3.2 Hz, 3H), 2.48 (s, 3H), 2.19-2.13 (m, 1H), 2.02 (d, J=8.0 Hz, 1H), 1.40 (d, J=7.2 Hz, 3H)

### Step 4: 8-methyl-2-(5-((3-methyl-1H-1,2,4-triazol-1-yl)methyl)-2-oxabicyclo[2.1.1]hexan-1-yl)-6-((R)-2-phenylpropoxy)-[1,2,4]triazolo[1,5-a]pyridine (Compound 487)

To a solution of [1-[8-methyl-6-[(2R)-2-phenylpropoxy]-[1,2,4]triazolo[1,5-a]pyridin-2-yl]-2-oxabicyclo[2.1.1]hexan5-yl]methyl 4-methylbenzenesulfonate (50 mg, 93.70 μmol, 1 eq) and 5-methyl-1H-1,2,4-triazole (9.34 mg, 112.44 μmol, 1.2 eq) in DMSO (1 mL) was added K₂CO₃ (25.90 mg, 187.39 μmol, 2 eq) and KI (15.55 mg, 93.70 μmol, 1 eq). The mixture was stirred at 80° C. for 1 h. LCMS and HPLC showed reactant was consumed completely and the desired compound was detected. The reaction was added H₂O (3 mL) and then extracted with EtOAc (15 mL×3). The combined organic phase was washed with brine (10 mL×3), dried over anhydrous Na₂SO₄, filtered and concentrated in vacuum. The residue was purified by prep-HPLC (column: Phenomenex C18 150 mm*30 mm*5 m; mobile phase: [water(FA)—ACN]; gradient:20%-50% B over 18 min) to give 8-methyl-2-(5-((3-methyl-1H-1,2,4-triazol-1-yl)methyl)-2-oxabicyclo[2.1.1]hexan-1-yl)-6-((R)-2-phenylpropoxy)-[1,2,4]triazolo[1,5-a]pyridine (8.37 mg, 16% yield, TFA) as a colorless gum.

LC-MS [ESI, M+1]: 445.2.

¹H NMR (400 MHz, CHLOROFORM-d) δ=8.81 (s, 1H), 7.97 (s, 1H), 7.35-7.29 (m, 2H), 7.27-7.19 (m, 3H), 7.13-7.07 (m, 1H), 4.40 (dd, J=14.4, 7.6 Hz, 1H), 4.12 (dd, J=4.8, 14.4 Hz, 1H), 4.04 (t, J=6.4 Hz, 2H), 3.97 (dd, J=7.2, 18.4 Hz, 2H), 3.24 (sxt, J=6.8 Hz, 1H), 3.08 (s, 1H), 2.87 (s, 1H), 2.51 (s, 3H), 2.39 (s, 3H), 2.17 (d, J=6.8 Hz, 1H), 2.01 (d, J=8.0 Hz, 1H), 1.40 (d, J=7.2 Hz, 3H).

| Compound | | |
|---|---|---|
| Number | Synthesis | LC/MS |
| 488 | Similar to | m/z 501.1 |
| | Example 55 | [M + H]⁺ |
| 489 | Similar to | m/z 421.3 |
| | Example 55 | [M + H]⁺ |
| 490 | Similar to | m/z 530.3 |
| | Example 55 | [M + H]⁺ |

### Example 56

### 8-methyl-6-[(3R)-3-methyl-3-phenyl-pyrrolidin-1-yl]-2-[(1R,4S,5R)-5-(3-methyl-1H-1,2,4-triazol-5-yl)-2-oxabicyclo[2.1.1]hexan-1-yl]-[1,2,4]triazolo[1,5-a]pyridine (Compound 491)

### 8-methyl-6-[(3R)-3-methyl-3-phenyl-pyrrolidin-1-yl]-2-[(1S,4R,5S)-5-(3-methyl-1H-1,2,4-triazol-5-yl)-2-oxabicyclo[2.1.1]hexan-1-yl]-[1,2,4]triazolo[1,5-a]pyridine (Compound 492)

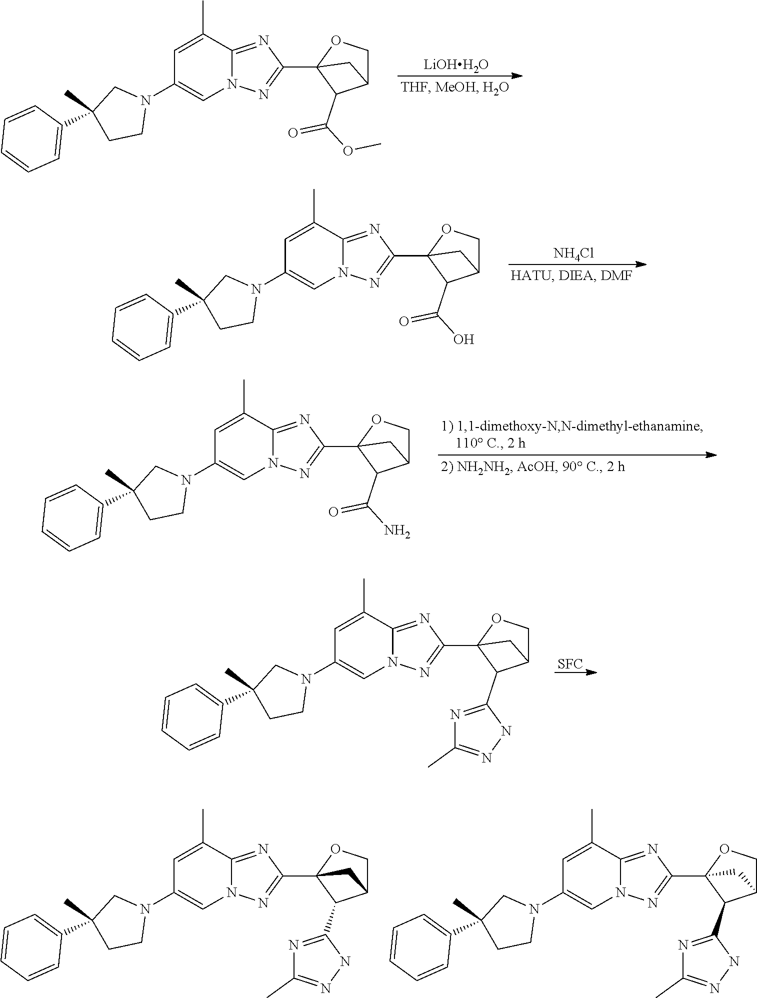

### Step 1: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid

To a solution of methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (200 mg, 462.41 μmol, 1 eq) in THF (1 mL), H₂O (1 mL) and MeOH (1 mL) was added LiOH·H₂O (58.21 mg, 1.39 mmol, 3 eq). The mixture was stirred at 25° C. for 2 h. LCMS showed starting material was consumed and the desired mass was detected. The mixture was added 2 M HCl to adjust pH=7, then extracted with ethyl acetate (20 mL×3), the combined organic phase was dried over anhydrous sodium sulfate, filtered and concentrated to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (190 mg, 98% yield) as a yellow solid.

LC-MS [ESI, M+1]: 419.2.

### Step 2: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (190 mg, 454.02 μmol, 1 eq) in DMF (2 mL) was added HATU (258.95 mg, 681.02 μmol, 1.5 eq) and DIEA (234.71 mg, 1.82 mmol, 316.33 μL, 4 eq) and NH₄Cl (97.14 mg, 1.82 mmol, 4 eq). LCMS showed starting material was consumed and the desired mass was detected. The reaction mixture was quenched by H₂O (5 mL), then extracted with ethyl acetate (10 mL×3), the combined organic layers dried over anhydrous sodium sulfate, filtered and the filtrate was concentrated under reduced pressure to give a residue. The residue was purified by prep-TLC (SiO₂, PE:EtOAc=0:1, Rf=0.4) to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (175 mg, 72% yield) as yellow oil.

LC-MS [ESI, M+1]: 418.2.

¹H NMR (400 MHz, CHLOROFORM-d) δ=7.63 (s, 1H), 7.41-7.34 (m, 2H), 7.33-7.29 (m, 2H), 7.27-7.23 (m, 1H), 7.01-6.93 (m, 1H), 4.15 (d, J=6.4 Hz, 1H), 4.05-3.98 (m, 1H), 3.68 (td, J=6.8, 13.2 Hz, 2H), 3.60 (br d, J=8.8 Hz, 1H), 3.55-3.45 (m, 2H), 3.39 (br s, 1H), 3.06 (d, J=3.2 Hz, 1H), 2.65-2.59 (m, 3H), 2.42-2.20 (m, 4H), 2.17 (br dd, J=2.8, 7.6 Hz, 1H), 1.47 (s, 3H).

### Step 3: 8-methyl-6-[(3R)-3-methyl-3-phenyl-pyrrolidin-1-yl]-2-[5-(3-methyl-11H-1,2,4-triazol-5-yl)-2-oxabicyclo[2.1.1]hexan-1-yl]-[1,2,4]triazolo[1,5-a]pyridine

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (220 mg, 526.94 μmol, 1 eq) in 1,1-dimethoxy-N,N-dimethyl-ethanamine (9.97 g, 74.83 mmol, 10.94 mL, 142 eq), the mixture was stirred at 110° C. for 2 hr. Then the mixture was concentrated under reduced pressure to give a residue. To a solution of this residue in EtOH (6 mL) was added hydrazine;hydrate (310.34 mg, 5.27 mmol, 300.72 μL, 85% purity, 10 eq) and AcOH (1.39 g, 23.19 mmol, 1.33 mL, 44 eq) at −10° C. And then the mixture was stirred at 90° C. for 2 h. LCMS showed starting material was consumed and the desired mass was detected. The reaction was concentrated under reduced pressure to give a residue. The residue was purified by prep-HPLC purification (mobile phase: [water(FA)—ACN]; B %: 29%-59%, 10 min), the mixture was concentrated and lyophilized to give compound 8-methyl-6-[(3R)-3-methyl-3-phenyl-pyrrolidin-1-yl]-2-[5-(3-methyl-1H-1,2,4-triazol-5-yl)-2-oxabicyclo[2.1.1]hexan-1-yl]-[1,2,4]triazolo[1,5-a]pyridine (68 mg, 28% yield) as a brown solid.

LC-MS [ESI, M+1]: 456.2.

¹H NMR (400 MHz, CHLOROFORM-d) δ=7.66 (s, 1H), 7.38 (br d, J=7.3 Hz, 2H), 7.35-7.31 (m, 2H), 7.28-7.25 (m, 1H), 7.01 (s, 1H), 4.00 (d, J=6.4 Hz, 1H), 3.83 (br d, J=6.4 Hz, 1H), 3.62 (br d, J=8.4 Hz, 1H), 3.57 (br s, 1H), 3.53 (br s, 1H), 3.49 (br d, J=8.8 Hz, 2H), 3.42 (br dd, J=3.6, 8.8 Hz, 1H), 2.66 (s, 3H), 2.44 (s, 4H), 2.38 (br s, 1H), 2.29 (br d, J=7.6 Hz, 1H), 2.21 (br d, J=7.6 Hz, 1H), 1.48 (s, 3H).

### Step 4: 8-methyl-6-[(3R)-3-methyl-3-phenyl-pyrrolidin-1-yl]-2-[(1R,4S,5R)-5-(3-methyl-1H-1,2,4-triazol-5-yl)-2-oxabicyclo[2.1.1]hexan-1-yl]-[1,2,4]triazolo[1,5-a]pyridine (Compound 491)

The 8-methyl-6-[(3R)-3-methyl-3-phenyl-pyrrolidin-1-yl]-2-[5-(3-methyl-1H-1,2,4-triazol-5-yl)-2-oxabicyclo[2.1.1]hexan-1-yl]-[1,2,4]triazolo[1,5-a]pyridine was purified by SFC purification ((column: DAICEL CHIRALPAK AS (250 mm*30 mm, 10 um);mobile phase: [C02-MeOH (0.1% NH₃H₂O)]; B %: 25%, isocratic elution mode), the mixture was concentrated and lophilizated to give compound 8-methyl-6-[(3R)-3-methyl-3-phenyl-pyrrolidin-1-yl]-2-[(1R,4S,5R)-5-(3-methyl-1H-1,2,4-triazol-5-yl)-2-oxabicyclo[2.1.1]hexan-1-yl]-[1,2,4]triazolo[1,5-a]pyridine (10.13 mg, 17% yield) as yellow oil.

LC-MS [ESI, M+1]: 456.2.

¹H NMR (400 MHz, CHLOROFORM-d) δ=7.65 (s, 1H), 7.42-7.28 (m, 4H), 7.25 (br s, 1H), 7.00 (br s, 1H), 3.99 (br d, J=6.0 Hz, 1H), 3.82 (br d, J=6.0 Hz, 1H), 3.60 (s, 1H), 3.56 (br s, 1H), 3.54-3.44 (m, 3H), 3.43-3.36 (m, 1H), 2.65 (s, 3H), 2.43 (s, 4H), 2.37 (br s, 1H), 2.30-2.25 (m, 1H), 2.20 (br d, J=7.6 Hz, 1H), 1.47 (s, 3H).

### Step 4:8-methyl-6-[(3R)-3-methyl-3-phenyl-pyrrolidin-1-yl]-2-[(1S,4R,5S)-5-(3-methyl-1H-1,2,4-triazol-5-yl)-2-oxabicyclo[2.1.1]hexan-1-yl]-[1,2,4]triazolo[1,5-a]pyridine (Compound 492)

The 8-methyl-6-[(3R)-3-methyl-3-phenyl-pyrrolidin-1-yl]-2-[5-(3-methyl-1H-1,2,4-triazol-5-yl)-2-oxabicyclo[2.1.1]hexan-1-yl]-[1,2,4]triazolo[1,5-a]pyridine was purified by SFC purification ((column: DAICEL CHIRALPAK AS (250 mm*30 mm, 10 um);mobile phase: [C02-MeOH (0.1% NH₃H₂O)]; B %: 25%, isocratic elution mode), the mixture was concentrated and lophilizated to give compound 8-methyl-6-[(3R)-3-methyl-3-phenyl-pyrrolidin-1-yl]-2-[(1S,4R,5S)-5-(3-methyl-1H-1,2,4-triazol-5-yl)-2-oxabicyclo[2.1.1]hexan-1-yl]-[1,2,4]triazolo[1,5-a]pyridine (13.14 mg, 22% yield) as white solid.

LC-MS [ESI, M+1]: 456.2.

¹H NMR (400 MHz, CHLOROFORM-d) δ=7.65 (br s, 1H), 7.36 (br d, J=7.2 Hz, 2H), 7.33-7.28 (m, 2H), 7.27 (br s, 1H), 7.00 (br s, 1H), 3.98 (br d, J=6.0 Hz, 1H), 3.81 (br d, J=5.6 Hz, 1H), 3.61 (br d, J=8.8 Hz, 1H), 3.56 (br s, 1H), 3.48 (br d, J=8.4 Hz, 3H), 3.40 (br s, 1H), 2.65 (br s, 3H), 2.43 (br s, 4H), 2.37 (br s, 1H), 2.27 (br d, J=3.6 Hz, 1H), 2.19 (br d, J=7.6 Hz, 1H), 1.47 (s, 3H).

| Compound | | |
|---|---|---|
| Number | Synthesis | LC/MS |
| 493 | Similar to | m/z 470.2 |
| | Example 56 | [M + H]⁺ |
| 494 | Similar to | m/z 442.1 |
| | Example 56 | [M + H]⁺ |
| 495 | Similar to | m/z 434.3 |
| | Example 56 | [M + H]⁺ |

### Example 57

### 5-(1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexan-5-yl)-1H-1,2,4-triazol-3-amine (Compound 496)

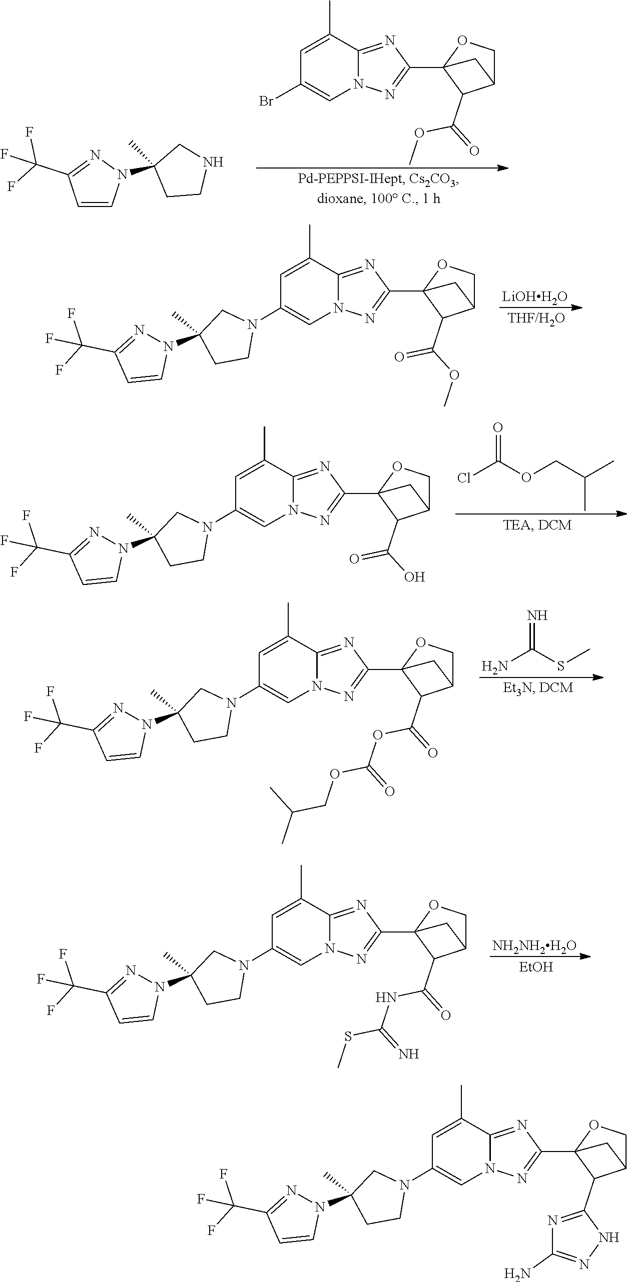

### Step 1: methyl 1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexane-5-carboxylate

To a solution of 1-[(3R)-3-methylpyrrolidin-3-yl]-3-(trifluoromethyl)pyrazole (30 mg, 136.86 μmol, 1 eq) and methyl 1-(6-bromo-8-methyl-[1,2,4]triazolo[1,5-a]pyridin-2-yl)-2-oxabicyclo[2.1.1]hexane-5-carboxylate (48.20 mg, 136.86 μmol, 1 eq) in dioxane (3 mL) was added 1,3-bis[2,6-bis(1-propylbutyl)phenyl]-4,5-dichloro-2H-imidazol-1-ium-2-ide;3-chloropyridine;dichloropalladium (6.66 mg, 6.84 μmol, 0.05 eq) and Cs₂CO₃ (133.77 mg, 410.57 μmol, 3 eq). The mixture was stirred at 100° C. for 12 h under N₂. LCMS showed starting material was consumed and one main peak with desired mass was detected. The reaction mixture was concentrated under the reduced pressure to give the product. The crude product was purified by reversed-phase HPLC (0.1% FA condition) to give compound methyl 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylate (38 mg, 57% yield) as a yellow solid.

LC-MS [ESI, M+1]: 491.5

### Step 2: 1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexane-5-carboxylic acid

To a solution of methyl 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylate (80 mg, 163.11 μmol, 1 eq) in THF (1 mL) was added LiOH (15.63 mg, 652.43 μmol, 4 eq) in H₂O (0.3 mL) was added at 25° C. The mixture was stirred at 25° C. for 0.5 h. LCMS showed starting material was consumed and one main peak with desired mass was detected. The reaction mixture was poured into 3 mL water and acidified with 2M HCl to pH around 4 and then extracted with ethyl acetate (10 mL×3).The combined organic phase was washed with brine (5 mL), dried with anhydrous Na₂SO₄, filtered and concentrated in vacuum to give compound 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylic acid (86 mg, crude) as a yellow gum.

LC-MS [ESI, M+1]: 477.1

### Step 3: (isobutyl carbonic) 1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexane-5-carboxylic anhydride

To a solution of 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylic acid (86 mg, 180.50 μmol, 1 eq) and TEA (54.79 mg, 541.50 μmol, 75.37 μL, 3 eq) in DCM (2 mL) was added isobutyl carbonochloridate (29.58 mg, 216.60 μmol, 28.34 μL, 1.2 eq) dropwised with stirring at −5° C., then the mixture stirred at 0° C. for 30 min. LCMS showed starting material was consumed and one main peak with desired mass was detected. The reaction mixture was concentrated under the reduced pressure to give the compound isobutoxycarbonyl 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylate (105 mg, crude) as a yellow liquid.

LC-MS [ESI, M+1]: 577.3.

### Step 4: methyl (1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexane-5-carbonyl)carbamimidothioate

To a solution of isobutoxycarbonyl 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexane-5-carboxylate (105 mg, 182.11 μmol, 1 eq), TEA (61.46 mg, 607.37 μmol, 84.54 μL, 3.34 eq) in DCM (2 mL) was added 2-methylisothiourea (182.52 mg, 2.02 mmol, 11.12 eq) in H₂O (0.6 mL) dropwised with stirring at 0° C., then the mixture stirred at 25° C. for 1 h. LCMS showed starting material was consumed and one main peak with desired mass was detected. The reaction was added water (5 mL) and then extracted with DCM (10 mL×3). The combined organic phase was washed with brine (5 mL×3), dried with anhydrous Na₂SO₄, filtered and concentrated in vacuum to give compound 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-N-(methylsulfanylcarbonimidoyl)-2-oxabicyclo[2.1.1]hexane-5-carboxamide (110 mg, crude) as a yellow gum.

LC-MS [ESI, M+1]: 549.3

### Step 5: 5-(1-(8-methyl-6-((R)-3-methyl-3-(3-(trifluoromethyl)-1H-pyrazol-1-yl)pyrrolidin-1-yl)-[1,2,4]triazolo[1,5-a]pyridin-2-yl)-2-oxabicyclo[2.1.1]hexan-5-yl)-1H-1,2,4-triazol-3-amine

To a solution of 1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-N-(methylsulfanylcarbonimidoyl)-2-oxabicyclo[2.1.1]hexane-5-carboxamide (110 mg, 200.52 μmol, 1 eq) in EtOH (1 mL) was added N₂H₄·H₂O (20.08 mg, 401.03 μmol, 19.45 μL, 100% purity, 2 eq). The mixture was stirred at 60° C. for 1 h under N₂ atmosphere. LCMS showed starting material was consumed and one main peak with desired mass was detected. The reaction was quenched by NH₄Cl (5 mL) slowly and then extracted with ethyl acetate (5 mL×3). The combined organic phase was washed with brine (5 mL), dried with anhydrous Na₂SO₄, filtered and concentrated in vacuum. The residue was purified by prep-HPLC (NH₄HCO₃ condition column: CD24-WePure Biotech XPT C18 150*25*7 um; mobile phase: [H2O (10 mM NH₄HCO₃)—ACN]; gradient:23%-53% B over 15.0 min) to give compound 5-[1-[8-methyl-6-[(3R)-3-methyl-3-[3-(trifluoromethyl)pyrazol-1-yl]pyrrolidin-1-yl]-[1,2,4]triazolo[1,5-a]pyridin-2-yl]-2-oxabicyclo[2.1.1]hexan-5-yl]-1H-1,2,4-triazol-3-amine (14.42 mg, 14% yield) as a white solid.

LC-MS [ESI, M+1]: 515.1.

¹H NMR (400 MHz, METHANOL-d4) δ=7.94 (dd, J=0.8, 2.4 Hz, 1H), 7.76 (br s, 1H), 7.19 (br s, 1H), 6.60 (d, J=2.4 Hz, 1H), 4.35-4.02 (m, 2H), 3.97 (br s, 1H), 3.63 (d, J=10.4 Hz, 1H), 3.57-3.35 (m, 4H), 2.97-2.78 (m, 1H), 2.58 (s, 3H), 2.50-2.35 (m, 2H), 2.08-1.85 (m, 1H), 1.77 (s, 3H).

### Example 58

### 4-(6-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methoxy)-2-azaspiro[3.3]heptan-2-yl)pyridin-2-ol (Compound 497)

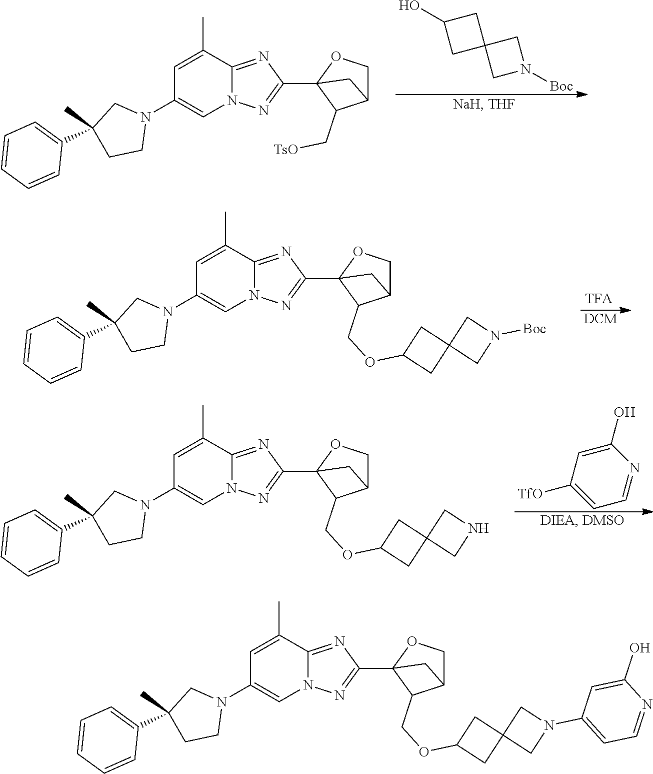

### Step 1: tert-butyl 6-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methoxy)-2-azaspiro[3.3]heptane-2-carboxylate

To a solution of tert-butyl 6-hydroxy-2-azaspiro[3.3]heptane-2-carboxylate (34.36 mg, 161.09 μmol, 3 eq) in THF (1 mL) was added NaH (4.30 mg, 107.39 μmol, 60% purity, 2 eq). The mixture was stirred at 25° C. for 1 h, and then [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl 4-methylbenzenesulfonate (30 mg, 53.70 μmol, 1 eq) was added 25° C. The mixture was stirred at 50° C. for 1 h. LCMS showed starting material was consumed and the desired mass was detected. The reaction was quenched by NH₄Cl (5 mL) slowly and then extracted with ethyl acetate (5 mL×3).The combined organic phase was washed with brine (5 mL), dried with anhydrous Na₂SO₄, filtered and concentrated in vacuum. The residue was purified by prep-TLC (SiO₂, PE:EtOAc=1:1, Rf=0.4) to give compound tert-butyl 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methoxy]-2-azaspiro[3.3]heptane-2-carboxylate (15 mg, 47% yield) as a yellow gum.

LC-MS [ESI, M+1]:600.4.

### Step 2: 2-(5-(((2-azaspiro[3.3]heptan-6-yl)oxy)methyl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of tert-butyl 6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methoxy]-2-azaspiro[3.3]heptane-2-carboxylate (60 mg, 100.04 μmol, 1 eq) in DCM (1 mL) was added TFA (767.50 mg, 6.73 mmol, 0.5 mL, 67.28 eq). The mixture was stirred at 25° C. for 1 h. LCMS showed starting material was consumed and the desired mass was detected. The reaction mixture was concentrated under the reduced pressure to give compound 2-[5-(2-azaspiro[3.3]heptan-6-yloxymethyl)-2-oxabicyclo[2.1.1]hexan-1-yl]-8-methyl-6-[(3R)-3-methyl-3-phenyl-pyrrolidin-1-yl]-[1,2,4]triazolo[1,5-a]pyridine (70 mg, crude, TFA) as a yellow gum.

LC-MS [ESI, M+1]:500.3.

### Step 3: 4-(6-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methoxy)-2-azaspiro[3.3]heptan-2-yl)pyridin-2-ol

To a solution of 2-[5-(2-azaspiro[3.3]heptan-6-yloxymethyl)-2-oxabicyclo[2.1.1]hexan-1-yl]-8-methyl-6-[(3R)-3-methyl-3-phenyl-pyrrolidin-1-yl]-[1,2,4]triazolo[1,5-a]pyridine (65 mg, 105.92 μmol, 1 eq, TFA) and (2-hydroxy-4-pyridyl) trifluoromethanesulfonate (25.76 mg, 105.92 μmol, 1 eq) in DMSO (1 mL) was added DIEA (41.07 mg, 317.76 μmol, 55.35 μL, 3 eq). The mixture was stirred at 100° C. for 1 h. LCMS showed starting material was consumed and the desired mass was detected. The reaction mixture was filtered. The residue was purified by prep-HPLC (NH₄HCO₃ condition column: Phenomenex C18 150*25 mm*10 um; mobile phase: [water(NH₄HCO₃)—ACN]; gradient:35%-65% B over 11 min) to give compound 4-[6-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methoxy]-2-azaspiro[3.3]heptan-2-yl]pyridin-2-ol (6.28 mg, 10.53 μmol, 10% yield) as a yellow solid.

LC-MS [ESI, M+1]:593.1.

¹H NMR (400 MHz, CHLOROFORM-d) δ=11.85-11.29 (m, 1H), 7.63 (s, 1H), 7.39-7.33 (m, 2H), 7.33-7.29 (m, 2H), 7.27-7.21 (m, 1H), 7.08 (d, J=7.2 Hz, 1H), 6.94 (s, 1H), 5.47 (br d, J=7.2 Hz, 1H), 5.15 (s, 1H), 4.00 (d, J=6.4 Hz, 1H), 3.92 (br d, J=6.0 Hz, 1H), 3.90-3.74 (m, 5H), 3.58 (d, J=8.8 Hz, 1H), 3.53-3.35 (m, 5H), 3.04 (br s, 1H), 2.62 (s, 4H), 2.52-2.42 (m, 2H), 2.40-2.32 (m, 1H), 2.27-2.19 (m, 2H), 2.16-2.07 (m, 2H), 1.94 (br d, J=7.6 Hz, 1H), 1.45 (s, 3H).

| Compound | | |
|---|---|---|
| Number | Synthesis | LC/MS |
| 498 | Similar to | m/z 462.1 |
| | Example 58 | [M + H]⁺ |
| 499 | Similar to | m/z 512.2 |
| | Example 58 | [M + H]⁺ |
| 500 | Similar to | m/z 498.1 |
| | Example 58 | [M + H]⁺ |
| 501 | Similar to | m/z 500.2 |
| | Example 58 | [M + H]⁺ |
| 502 | Similar to | m/z 609.2 |
| | Example 58 | [M + H]⁺ |
| 503 | Similar to | m/z 609.3 |
| | Example 58 | [M + H]⁺ |
| 504 | Similar to | m/z 457.2 |
| | Example 58 | [M + H]⁺ |

### Example 59

### 2-(5-((4H-1,2,4-triazol-3-yl)methyl)-2-oxabicyclo[2.1.1]hexan-1-yl)-8-methyl-6-((R)-3-methyl-3-phenylpyrrolidin-1-yl)-[1,2,4]triazolo[1,5-a]pyridine (Compound 505)

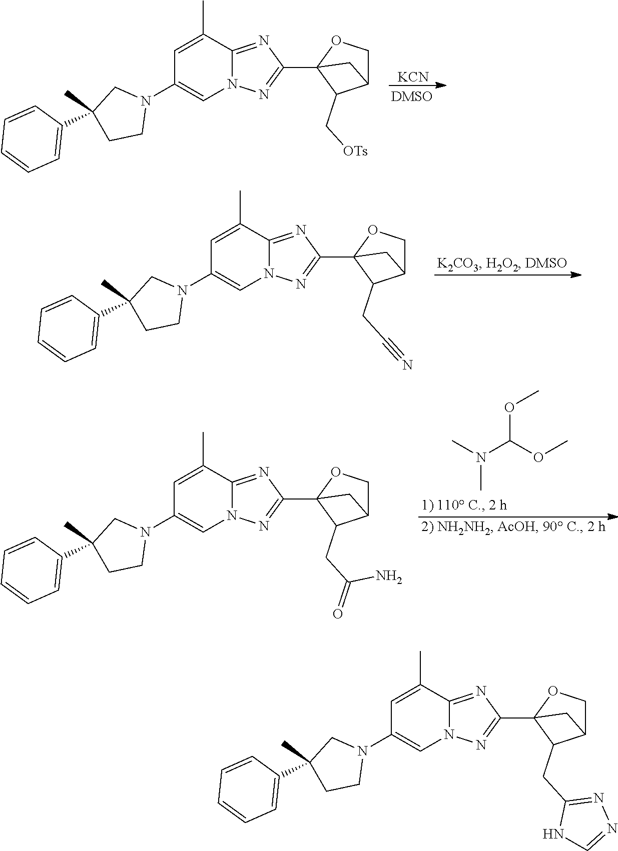

### Step 1: 2-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)acetonitrile

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl 4-methylbenzenesulfonate (400 mg, 715.96 μmol, 1 eq) in DMSO (3 mL) was added KCN (186.48 mg, 2.86 mmol, 122.68 μL, 4 eq) slowly. The mixture was stirred at 100° C. for 3 h. LCMS showed starting material was consumed and the desired mass was detected. The reaction was added water (10 mL) and then extracted with ethyl acetate (10 mL×3). The combined organic phase was washed with brine (10 mL), dried over anhydrous Na₂SO₄, filtered and concentrated in vacuo to give compound 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]acetonitrile (266 mg, 83% yield) as a yellow gum.

LC-MS [ESI, M+1]: 414.2.

### Step 2: 2-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)acetamide

To a solution of 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]acetonitrile (266 mg, 643.27 μmol, 1 eq) and K₂CO₃ (88.90 mg, 643.27 μmol, 1 eq) in DCM (3 mL) was added dropwise H₂O₂ (2.19 g, 19.30 mmol, 1.85 mL, 30% purity, 30 eq) at 0° C. The mixture was stirred at 25° C. for 0.5 h. LCMS showed starting material was consumed and the desired mass was detected. The reaction was quenched by Sodium Nitrite aq. (10 mL) slowly and then extracted with Dichloromethane (10 mL×3). The combined organic phases were dried over Na₂SO₄,filtered and concentrated in vacuum. The residue was purified by prep-HPLC (NH₄HCO₃ condition column: Waters xbridge 150*25 mm 10 um; mobile phase: [water(NH₄HCO₃)—ACN]; gradient:24%-54% B over 10 min) to give compound 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]acetamide (168.09 mg, 61% yield) as a white solid.

LC-MS [ESI, M+1]: 432.1

¹H NMR (400 MHz, CHLOROFORM-d) δ=7.61 (s, 1H), 7.57 (br s, 1H), 7.41-7.32 (m, 2H), 7.32-7.28 (m, 2H), 7.27-7.22 (m, 1H), 6.95 (s, 1H), 5.26 (br s, 1H), 3.98 (d, J=6.4 Hz, 1H), 3.85 (d, J=6.4 Hz, 1H), 3.59 (d, J=8.8 Hz, 1H), 3.55-3.44 (m, 2H), 3.38 (dt, J=3.6, 8.8 Hz, 1H), 2.98 (t, J=2.8 Hz, 1H), 2.79-2.64 (m, 2H), 2.58 (s, 3H), 2.37 (td, J=8.4, 12.0 Hz, 1H), 2.30-2.09 (m, 3H), 2.00 (d, J=7.6 Hz, 1H), 1.46 (s, 3H)

### Step 3: 2-(5-((4H-1,2,4-triazol-3-yl)methyl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of 2-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]acetamide (50 mg, 115.87 μmol, 1 eq) in 1,1-dimethoxy-N,N-dimethyl-methanamine (897.00 mg, 7.53 mmol, 1 mL, 64.97 eq), the mixture was stirred at 110° C. for 2 h. Then the mixture was concentrated under reduced pressure to give a residue. To a solution of this residue in EtOH (1 mL) was added HOAc (209.80 mg, 3.49 mmol, 0.2 mL, 30.15 eq) and N₂H₄·H₂O (206.40 mg, 3.50 mmol, 0.2 mL, 85% purity, 30.25 eq) at −10° C. And then the mixture was stirred at 90° C. for 2 h. LCMS showed starting material was consumed and the desired mass was detected. The reaction was quenched by ice-water (5 mL) slowly and then extracted with ethyl acetate (5 mL×3).The combined organic phase was washed with brine (5 mL), dried with anhydrous Na₂SO₄, filtered and concentrated in vacuum. The residue was purified by prep-HPLC (FA condition column: Phenomenex Luna C18 150*25 mm*10 um; mobile phase: [water(FA)—ACN]; gradient:29%-59% B over 10 min) to give compound 8-methyl-6-[(3R)-3-methyl-3-phenyl-pyrrolidin-1-yl]-2-[5-(4H-1,2,4-triazol-3-ylmethyl)-2-oxabicyclo[2.1.1]hexan-1-yl]-[1,2,4]triazolo[1,5-a]pyridine (17.1 mg, 32% yield) as a white solid.

LC-MS [ESI, M+1]: 456.2

¹H NMR (400 MHz, CHLOROFORM-d) δ=7.85 (s, 1H), 7.66 (br s, 1H), 7.44-7.34 (m, 2H), 7.33-7.29 (m, 2H), 7.26-7.22 (m, 1H), 7.03 (br s, 1H), 4.06 (br d, J=6.4 Hz, 1H), 3.96 (br d, J=6.4 Hz, 1H), 3.61 (br d, J=8.8 Hz, 1H), 3.56-3.44 (m, 2H), 3.44-3.36 (m, 1H), 3.17 (br dd, J=9.2, 16.0 Hz, 1H), 3.02-2.88 (m, 2H), 2.76 (s, 3H), 2.68 (br d, J=9.2 Hz, 1H), 2.43-2.34 (m, 1H), 2.31-2.23 (m, 1H), 2.12-2.03 (m, 2H), 1.47 (s, 3H).

### Example 60

### 3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methyl)-3-azabicyclo[3.1.0]hexane-6-carboxylic acid (Compound 506)

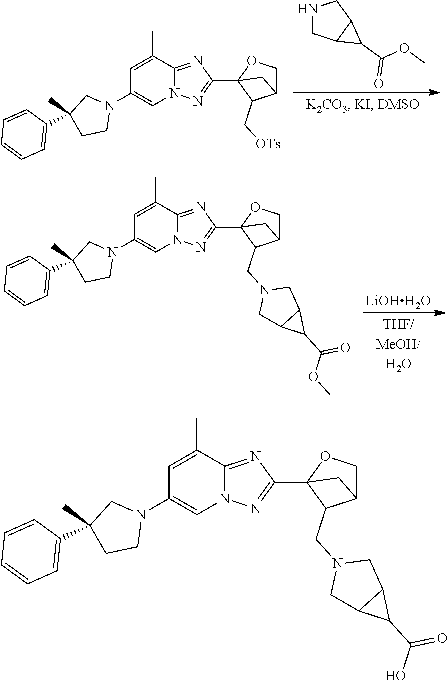

### Step 1: methyl 3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methyl)-3-azabicyclo[3.1.0]hexane-6-carboxylate

To a solution of [1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl 4-methylbenzenesulfonate (50 mg, 89.50 μmol, 1 eq) and methyl 3-azabicyclo[3.1.0]hexane-6-carboxylate (23.85 mg, 134.24 μmol, 1.5 eq, HCl) in DMSO (0.5 mL) was added KI (14.86 mg, 89.50 μmol, 1 eq) and K₂CO₃ (37.11 mg, 268.49 μmol, 3 eq). The mixture was stirred at 70° C. for 1 hr. LCMS showed starting material was consumed and one main peak with desired mass was detected. The reaction mixture was added H₂O (10 ml) and then was extracted with EtOAc (10 mL×3), the combined organic layers were washed with brine (10 mL×3), dried over anhydrous Na₂SO₄, filtered and dried to give a residue. the residue was purified by prep-TLC (SiO₂, PE:EtOAc=0:1, Rf=0.2) and eluted with 60 mL (10% methanol in DCM) to give compound methyl 3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methyl)-3-azabicyclo[3.1.0]hexane-6-carboxylate (20 mg, 37.90 μmol, 42.35% yield) as a white gum.

LC-MS [ESI, M+1]:528.1

### Step 2: 3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methyl)-3-azabicyclo[3.1.0]hexane-6-carboxylic acid

To a solution of methyl 3-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methyl]-3-azabicyclo[3.1.0]hexane-6-carboxylate (20 mg, 37.90 μmol, 1 eq) in THF (1 mL) was added LiOH·H₂O (4.77 mg, 113.71 μmol, 3 eq), the mixture was stirred at 25° C. for 1 hr. LCMS showed starting material was consumed and one main peak with desired mass was detected. The residue was purified by prep-HPLC (column: Phenomenex luna C18 150*25 mm*10 um; mobile phase: [water(FA)—ACN]; gradient:18%-48% B over 10 min) and lyophilized to give compound 3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methyl)-3-azabicyclo[3.1.0]hexane-6-carboxylic acid (3.02 mg, 5.29 μmol, 13.95% yield, 97.970% purity, FA) as a yellow solid.

LC-MS [ESI, M+1]:528.1

¹H NMR (DMSO-d₆, 400 MHz): δ=8.42 (s, 1H), 7.86 (s, 1H), 7.28-7.43 (m, 4H), 7.21 (d, J=11.2 Hz, 2H), 3.74-3.78 (m, 2H), 3.55 (s, 4H), 3.50 (s, 3H), 2.96 (dd, J=16.4, 8.8 Hz, 2H), 2.87 (s, 1.0H), 2.47-2.49 (m, 3.1H), 2.42 (d, J=6.4 Hz, 1H), 2.29 (d, J=7.2 Hz, 1H), 2.24 (t, J=6.4 Hz, 2H), 1.97 (br d, J=4.4 Hz, 1H), 1.61-1.81 (m, 4H), 1.35 (s, 3H)

### Example 61

### 6-(1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)-4,6-diazaspiro[2.4]heptan-5-one

### tert-butyl (1-(6-bromo-8-methyl-[1,2,4]triazolo[1,5-a]pyridin-2-yl)-2-oxabicyclo[2.1.1]hexan-5-yl)carbamate (Compound 507)

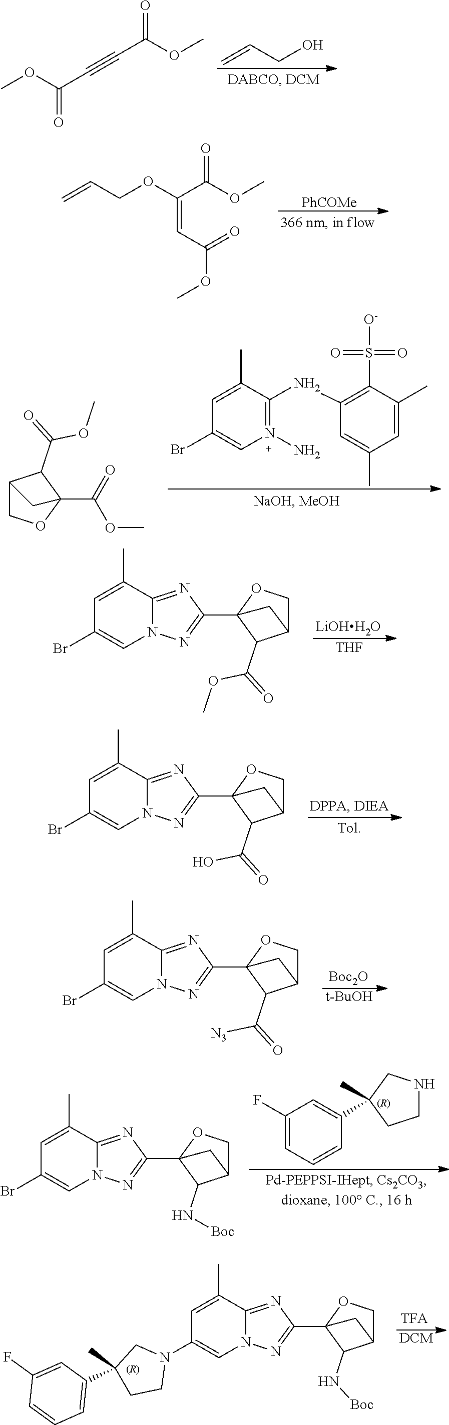

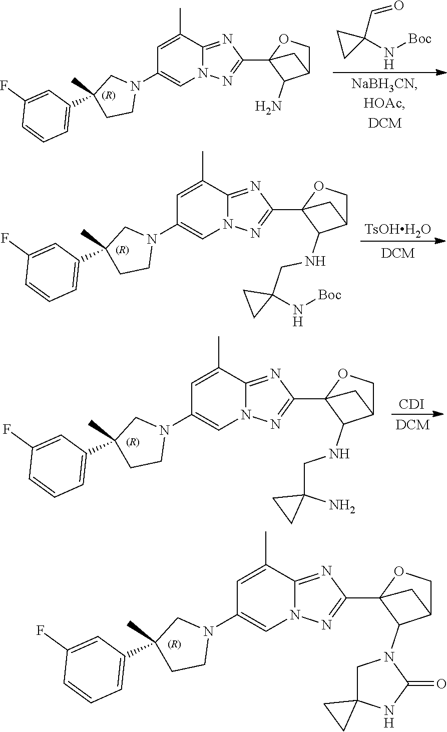

### Step 1: dimethyl (E)-2-allyloxybut-2-enedioate

To a solution of dimethyl but-2-ynedioate (50 g, 351.84 mmol, 1 eq) in DCM (200 mL) was added prop-2-en-1-ol (21.46 g, 369.43 mmol, 25.12 mL, 1.05 eq). Then a solution of DABCO (3.95 g, 35.18 mmol, 3.87 mL, 0.1 eq) in DCM (30 mL) was added dropwise. The mixture was stirred at 25° C. for 3 h. TLC (PE:EtOAc=5:1) showed starting material was not consumed completely and several new spots formed. The reaction was concentrated under reduced pressure to give a residue. The residue was purified by column chromatography (SiO₂, PE:EtOAc=5:1, Rf=0.45) to give compound dimethyl (E)-2-allyloxybut-2-enedioate (130 g, 46% yield) as a colourless liquid.

¹H NMR (400 MHz, CHLOROFORM-d) δ=5.97-5.86 (m, 1H), 5.41-5.26 (m, 2H), 5.17 (s, 1H), 4.39 (d, J=5.2 Hz, 2H), 3.85 (s, 3H), 3.66 (s, 3H).

### Step 2: dimethyl 2-oxabicyclo[2.1.1]hexane-1,5-dicarboxylate

To a solution of dimethyl (E)-2-allyloxybut-2-enedioate (120 g, 599.44 mmol, 1 eq) in ACN (1000 mL) was added diphenylmethanone (10.92 g, 59.94 mmol, 0.1 eq). The mixture was stirred at 25° C. (366 nm, in flow) for 1 h. TLC (PE:EtOAc=5:1) showed starting material was not consumed completely and three new spots formed. The reaction mixture was concentrated in vacuum to give compound dimethyl 2-oxabicyclo[2.1.1]hexane-1,5-dicarboxylate (120 g, crude) as a yellow liquid.

### Step 3: methyl 1-(6-bromo-8-methyl-[1,2,4]triazolo[1,5-a]pyridin-2-yl)-2-oxabicyclo[2.1.1]hexane-5-carboxylate

To a solution of dimethyl 2-oxabicyclo[2.1.1]hexane-1,5-dicarboxylate (37.82 g, 188.91 mmol, 2 eq) and 5-bromo-3-methyl-pyridin-1-ium-1,2-diamine;2,4,6-trimethylbenzenesulfonate (38 g, 94.46 mmol, 1 eq) in MeOH (500 mL) was added NaOH (5.67 g, 141.68 mmol, 1.5 eq). The mixture was stirred at 70° C. for 1 h. LCMS showed starting material was consumed completely and the desired compound was detected. The reaction was concentrated under reduced pressure to give a residue. The residue was purified by column chromatography (SiO₂, PE:EtOAc=1:1, Rf=0.19) to give compound methyl 1-(6-bromo-8-methyl-[1,2,4]triazolo[1,5-a]pyridin-2-yl)-2-oxabicyclo[2.1.1]hexane-5-carboxylate (8 g, 24% yield) as a yellow solid.

LC-MS [ESI, M+1]: 352.5.

¹H NMR (400 MHz, DMSO-d6) δ=9.18 (d, J=0.4 Hz, 1H), 7.68 (s, 1H), 4.02-3.98 (m, 1H), 3.86 (d, J=6.4 Hz, 1H), 3.50 (s, 3H), 3.32 (s, 1H), 3.23 (t, J=2.8 Hz, 1H), 2.53 (s, 3H), 2.19 (dd, J=7.2, 2.8 Hz, 1H), 1.70 (d, J=7.2 Hz, 1H).

### Step 4: 1-(6-bromo-8-methyl-[1,2,4]triazolo[1,5-a]pyridin-2-yl)-2-oxabicyclo[2.1.1]hexane-5-carboxylic acid

To a solution of methyl 1-(6-bromo-8-methyl-[1,2,4]triazolo[1,5-a]pyridin-2-yl)-2-oxabicyclo[2.1.1]hexane-5-carboxylate (4 g, 11.36 mmol, 1 eq) in THF (30 mL) and H₂O (10 mL) was added LiOH·H₂O (1.43 g, 34.07 mmol, 3 eq). The mixture was stirred at 25° C. for 12 h. LCMS showed starting material was consumed and the desired mass was detected. The pH was adjusted to around 4 by aqueous. FA, and then extracted with ethyl acetate (50 mL×2). The combined organic layers were washed with brine (50 mL), dried over Na₂SO₄, filtered and concentrated under reduced pressure to give compound 1-(6-bromo-8-methyl-[1,2,4]triazolo[1,5-a]pyridin-2-yl)-2-oxabicyclo[2.1.1]hexane-5-carboxylic acid (3.7 g, 10.83 mmol, 95.37% yield, 99% purity) as yellow solid.

LC-MS [ESI, M+1]:339.9.

¹H NMR (400 MHz, METHANOL-d4) δ=8.87 (s, 1H), 7.64 (s, 1H), 4.23 (d, J=6.0 Hz, 1H), 3.99 (d, J=6.0 Hz, 1H), 3.37 (d, J=2.8 Hz, 1H), 3.30 (br s, 1H), 2.62 (s, 3H), 2.29 (dd, J=2.8, 7.2 Hz, 1H), 1.81 (d, J=7.6 Hz, 1H)

### Step 5: 1-(6-bromo-8-methyl-[1,2,4]triazolo[1,5-a]pyridin-2-yl)-2-oxabicyclo[2.1.1]hexane-5-carbonyl azide

To a solution of 1-(6-bromo-8-methyl-[1,2,4]triazolo[1,5-a]pyridin-2-yl)-2-oxabicyclo[2.1.1]hexane-5-carboxylic acid (2.5 g, 7.39 mmol, 1 eq) in toluene. (25 mL) was added DPPA (2.24 g, 8.13 mmol, 1.76 mL, 1.1 eq) and DIEA (1.05 g, 8.13 mmol, 1.42 mL, 1.1 eq). The mixture was stirred at 25° C. for 2 h. LCMS showed starting material was consumed and the desired mass was detected. The mixture was quenched with water (50 mL) and extracted with ethyl acetate (80 mL×2). The combined organic phase was washed with brine (50 mL), dried with anhydrous Na₂SO₄, filtered and concentrated in vacuum to give compound 1-(6-bromo-8-methyl-[1,2,4]triazolo[1,5-a]pyridin-2-yl)-2-oxabicyclo[2.1.1]hexane-5-carbonyl azide (2.8 g, crude) as yellow oil.

LC-MS [ESI, M-28+1]:336.9.

### Step 6: tert-butyl (1-(6-bromo-8-methyl-[1,2,4]triazolo[1,5-a]pyridin-2-yl)-2-oxabicyclo[2.1.1]hexan-5-yl)carbamate

To a solution of 1-(6-bromo-8-methyl-[1,2,4]triazolo[1,5-a]pyridin-2-yl)-2-oxabicyclo[2.1.1]hexane-5-carbonyl azide (2.8 g, 7.71 mmol, 1 eq) in t-BuOH (30 mL) was added Boc₂O (3.37 g, 15.42 mmol, 3.54 mL, 2 eq). The mixture was stirred at 90° C. for 1 h. LCMS showed starting material was consumed and the desired mass was detected. The reaction mixture was extracted with ethyl acetate (80 mL×2). The combined organic phase was washed with brine (50 mL), dried with anhydrous Na₂SO₄, filtered and concentrated in vacuum. The residue was purified by column chromatography (SiO₂, PE:EtOAc=1:1, Rf=0.4) to give compound tert-butyl N-[1-(6-bromo-8-methyl-[1,2,4]triazolo[1,5-a]pyridin-2-yl)-2-oxabicyclo[2.1.1]hexan-5-yl]carbamate (1.7 g, 3.99 mmol, 51.72% yield, 96% purity) as yellow solid.

LC-MS [ESI, M+1]:410.9.

¹H NMR (400 MHz, CHLOROFORM-d) δ=8.54 (d, J=0.8 Hz, 1H), 7.39 (s, 1H), 5.22 (br d, J=6.8 Hz, 1H), 4.22-4.11 (m, 1H), 4.00 (d, J=6.4 Hz, 1H), 3.90 (br d, J=6.4 Hz, 1H), 3.11 (br s, 1H), 2.65 (s, 3H), 2.13 (br dd, J=2.8, 8.4 Hz, 1H), 1.84 (d, J=8.4 Hz, 1H), 1.41 (s, 9H).

### Step 7: tert-butyl (1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)carbamate

To a solution of tert-butyl N-[1-(6-bromo-8-methyl-[1,2,4]triazolo[1,5-a]pyridin-2-yl)-2-oxabicyclo[2.1.1]hexan-5-yl]carbamate (300 mg, 733.00 μmol, 1 eq) and (3R)-3-(3-fluorophenyl)-3-methyl-pyrrolidine (262.76 mg, 1.47 mmol, 2 eq) in dioxane (5 mL) was added 1,3-bis[2,6-bis(1-propylbutyl)phenyl]-4,5-dichloro-2H-imidazol-1-ium-2-ide;3-chloropyridine;dichloropalladium (35.65 mg, 36.65 μmol, 0.05 eq) and Cs₂CO₃ (716.48 mg, 2.20 mmol, 3 eq). The mixture was stirred at 100° C. for 1 h. LCMS showed starting material was consumed and one main peak with desired mass was detected. The reaction mixture was added H₂O (10 ml) extracted with EtOAc (10 mL×3), the combined organic layers were dried over anhydrous Na₂SO₄, filtered and dried to give a residue. The residue was purified by flash silica gel chromatography (PE:EtOAc=0:1, Rf=0.6) to give compound tert-butyl (1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)carbamate (300 mg, 591.02 μmol, 80.63% yield) as a white solid.

LC-MS [ESI, M+1]: 508.3

¹H NMR (CHLOROFORM-d, 400 MHz): δ=7.55 (s, 1H), 7.13-7.36 (m, 2H), 6.88-7.05 (m, 2H), 6.81-6.87 (m, 1H), 3.77-3.94 (m, 2H), 3.27-3.66 (m, 4H), 2.88-3.09 (m, 1H), 2.47-2.65 (m, 3H), 2.21-2.35 (m, 1H), 2.16 (dd, J=7.2, 4.2 Hz, 1H), 2.03 (d, J=5.6 Hz, 1H), 1.77 (d, J=8.4 Hz, 1H), 1.26-1.42 (m, 12H)

### Step 8: 1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-amine

To a solution of tert-butyl N-[1-[6-[(3R)-3-(3-fluorophenyl)-3-methyl-pyrrolidin-1-yl]-8-methyl-[1,2,4]triazolo[1,5-a]pyridin-2-yl]-2-oxabicyclo[2.1.1]hexan-5-yl]carbamate (280 mg, 551.62 μmol, 1 eq) in DCM (4 mL) was added TFA (62.90 mg, 551.62 μmol, 40.97 μL, 1 eq). The mixture was stirred at 25° C. for 1 h. LCMS showed starting material was consumed and one main peak with desired mass was detected. The reaction mixture was added H₂O (10 ml) and then was extracted with DCM (10 mL×3), dried over anhydrous Na₂SO₄, filtered and dried to give compound 1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-amine (200 mg, crude) as a yellow gum.

LC-MS [ESI, M+1]: 408.5

### Step 9: tert-butyl (1-(((1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)amino)methyl)cyclopropyl)carbamate

To a solution of 1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-amine (80 mg, 196.33 μmol, 1 eq) and tert-butyl N-(1-formylcyclopropyl)carbamate (72.73 mg, 392.65 μmol, 2 eq) in DCM (3 mL) was added NaBH₃CN (37.01 mg, 588.98 μmol, 3 eq). The mixture was stirred at 25° C. for 1 h. LCMS showed starting material was consumed and one main peak with desired mass was detected. The reaction mixture was concentrated under the reduced pressure to give compound tert-butyl (1-(((1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)amino)methyl)cyclopropyl)carbamate (60 mg, 104.04 μmol, 52.99% yield) as a brown gum.

LC-MS [ESI, M+1]: 577.4

### Step 10: N-((1-aminocyclopropyl)methyl)-1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-amine

To a solution of tert-butyl N-[1-[[[1-[6-[(3R)-3-(3-fluorophenyl)-3-methyl-pyrrolidin-1-yl]-8-methyl-[1,2,4]triazolo[1,5-a]pyridin-2-yl]-2-oxabicyclo[2.1.1]hexan-5-yl]amino]methyl]cyclopropyl]carbamate (50 mg, 86.70 μmol, 1 eq) in DCM (3 mL) was added TsOH·H₂O (49.48 mg, 260.10 μmol, 3 eq). The mixture was stirred at 40° C. for 15 min. LCMS showed starting material was consumed and one main peak with desired mass was detected. The mixture was concentrated to give residue, the residue was adjusted pH around 7 by NaHCO₃,solid was precipitate out, and the mixture was filtered and the solid was concentrated under reduced pressure to give compound N-((1-aminocyclopropyl)methyl)-1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-amine (41 mg, 86.03 μmol, 99.23% yield) as a white gum.

LC-MS [ESI, M+1]: 477.2

### Step 11: 6-(1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)-4,6-diazaspiro[2.4]heptan-5-one

To a solution of N-[(1-aminocyclopropyl)methyl]-1-[6-[(3R)-3-(3-fluorophenyl)-3-methyl-pyrrolidin-1-yl]-8-methyl-[1,2,4]triazolo[1,5-a]pyridin-2-yl]-2-oxabicyclo[2.1.1]hexan-5-amine (35 mg, 73.44 μmol, 1 eq) in DCM (2 mL) was added CDI (59.54 mg, 367.19 μmol, 5 eq) The mixture was stirred at 40° C. for 1 hr. LCMS showed starting material was consumed and one main peak with desired mass was detected. The reaction mixture was concentrated under the reduced pressure to give the product. The residue was purified by prep-HPLC (column: CD01-Phenomenex luna C18 150*25*10 um;mobile phase: [water(FA)—ACN]; gradient:38%-68% B over 8 min) and lyophilized to give compound 6-(1-(6-((R)-3-(3-fluorophenyl)-3-methylpyrrolidin-1-yl)-8-methyl-[1,2,4]triazolo[1,5-a]pyridin-2-yl)-2-oxabicyclo[2.1.1]hexan-5-yl)-4,6-diazaspiro[2.4]heptan-5-one (5.72 mg, 10.87 μmol, 14.81% yield, 95.540% purity) as a white solid

LC-MS [ESI, M+1]: 503.3

¹H NMR (CHLOROFORM-d, 400 MHz): δ=7.63 (s, 1H), 7.27-7.39 (m, 2H), 6.95-7.12 (m, 2H), 6.94 (s, 1H), 4.08 (s, 2H), 3.92-4.05 (m, 2H), 3.51-3.63 (m, 3H), 3.48 (d, J=16.4 Hz, 1H), 3.43 (s, 2H), 2.62 (s, 3H), 2.30-2.44 (m, 1H), 2.25 (dd, J=6.8, 4.2 Hz, 1H), 2.11 (dd, J=7.6, 2.4 Hz, 1H), 1.93 (d, J=8.1 Hz, 1H), 1.46 (s, 3H), 0.60-0.78 (m, 3H), 0.54 (d, J=10.4 Hz, 1H)

| Compound | | |
|---|---|---|
| Number | Synthesis | LC/MS |
| 508 | Similar to | m/z 408.2 |
| | Example 61 | [M + H]⁺ |
| 509 | Similar to | m/z 501.3 |
| | Example 61 | [M + H]⁺ |
| 510 | Similar to | m/z 475.7 |
| | Example 61 | [M + H]⁺ |
| 511 | Similar to | m/z 529.4 |
| | Example 61 | [M + H]⁺ |

### Example 62

### 6-[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]-4,6-diazaspiro[2.4]heptan-5-one (Compound 512)

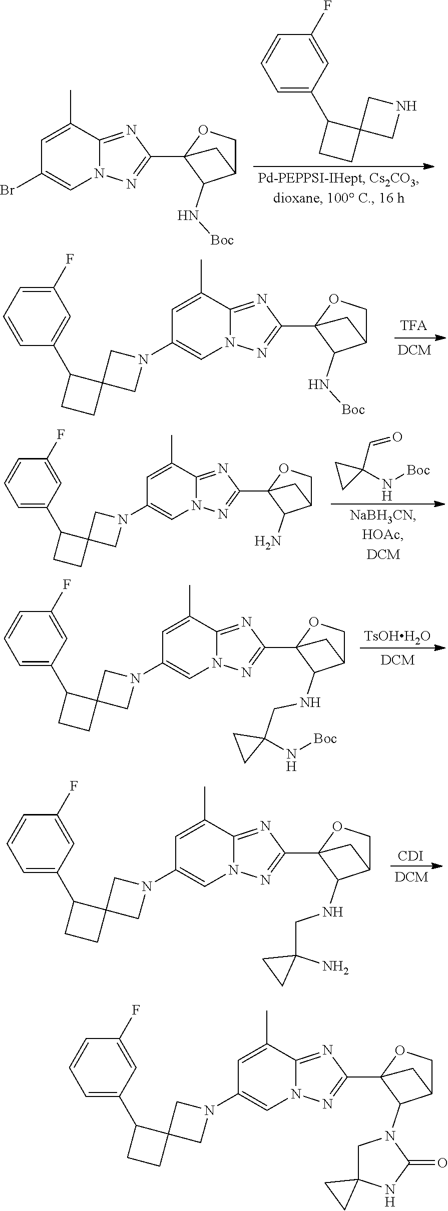

### Step 1: tert-butyl N-[1-[6-[7-(3-fluorophenyl)-2-azaspiro[3.3]heptan-2-yl]-8-methyl-[1,2,4]triazolo[1,5-a]pyridin2-yl]-2-oxabicyclo[2.1.1]hexan-5-yl]carbamate

A mixture of tert-butyl N-[1-(6-bromo-8-methyl-[1,2,4]triazolo[1,5-a]pyridin-2-yl)-2-oxabicyclo[2.1.1]hexan-5-yl]carbamate (220 mg, 537.53 μmol, 1 eq), 7-(3-fluorophenyl)-2-azaspiro[3.3]heptane (205.60 mg, 1.08 mmol, 2 eq), CS₂CO₃ (525.42 mg, 1.61 mmol, 3 eq) and 1,3-bis[2,6-bis(1-propylbutyl)phenyl]-4,5-dichloro-2H-imidazol-1-ium-2-ide;3-chloropyridine;dichloropalladium (52.29 mg, 53.75 μmol, 0.1 eq) in 1,4-dioxane (3 mL) was degassed and purged with N₂ for 3 times, and then the mixture was stirred at 100° C. for 1 h under N₂ atmosphere. LCMS showed starting material was consumed and the desired mass was detected. The reaction was filtered and the filtered concentrated under reduced pressure to give a residue. The residue was purified by flash silica gel chromatography (SiO₂, PE:EtOAc=0:1, Rf=0.3) to give compound tert-butyl N-[1-[6-[7-(3-fluorophenyl)-2-azaspiro[3.3]heptan-2-yl]-8-methyl-[1,2,4]triazolo[1,5-a]pyridin2-yl]-2-oxabicyclo[2.1.1]hexan-5-yl]carbamate (270 mg, 96.67% yield) as a white solid.

LC-MS [ESI, M+1]: 520.1

¹H NMR (400 MHz, CHLOROFORM-d) δ=7.48-7.38 (m, 1H), 7.36-7.29 (m, 1H), 7.07-6.84 (m, 3H), 6.68-6.54 (m, 1H), 5.33-5.22 (m, 2H), 4.10 (br d, J=7.6 Hz, 1H), 4.02-3.93 (m, 1H), 3.93-3.85 (m, 2H), 3.71 (s, 2H), 3.64-3.57 (m, 1H), 3.08 (br s, 1H), 2.64 (d, J=11.4 Hz, 1H), 2.55 (s, 2H), 2.38-2.05 (m, 4H), 1.92-1.78 (m, 1H), 1.69 (br s, 2H), 1.40 (br s, 9H).

### Step 2:1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine

To a solution of tert-butyl N-[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]carbamate (200 mg, 384.90 μmol, 1 eq) in DCM (2 mL) was added TFA (438.88 mg, 3.85 mmol, 285.92 μL, 10 eq). The mixture was stirred at 25° C. for 2 h. LCMS showed starting material was consumed and the desired mass was detected. The reaction was filtered and the filtered concentrated under reduced pressure to give compound 1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine (crude, TFA) as a white solid.

LC-MS [ESI, M+1]:420.2.

### Step 3: tert-butyl N-[1-[[[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]amino]methyl]cyclopropyl]carbamate

To a solution of 1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine (300 mg, 715.15 μmol, 1 eq) and tert-butyl N-(1-formylcyclopropyl)carbamate (132.46 mg, 715.15 μmol, 1 eq) in MeOH (2 mL) was added sodium; cyanoboranuide (89.88 mg, 1.43 mmol, 2 eq) and acetic acid (21.47 mg, 357.57 μmol, 20.47 μL, 0.5 eq). The mixture was stirred at 0-25° C. for 2 h. LCMS showed starting material was consumed and the desired mass was detected. The reaction was filtered and the filtered concentrated under reduced pressure to give a residue. The residue was purified by prep-HPLC (column: CD01-Phenomenex luna C18 150*25*10 um; mobile phase: [water(FA)—ACN]; gradient:25%-55% B over 8 min). The result solution was lyophilized to give compound tert-butyl N-[1-[[[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]amino]methyl]cyclopropyl]carbamate (150 mg, 35.63% yield) as a white solid.

LC-MS [ESI, M+1]: 589.3.

¹H NMR (400 MHz, CHLOROFORM-d) δ=7.43 (s, 1H), 7.35-7.26 (m, 1H), 7.00 (br d, J=7.6 Hz, 1H), 6.97-6.89 (m, 2H), 6.63 (s, 1H), 3.95 (s, 2H), 3.93-3.85 (m, 2H), 3.65-3.58 (m, 2H), 3.54 (br d, J=7.2 Hz, 1H), 3.30 (br s, 2H), 3.16-3.07 (m, 1H), 2.98 (br s, 1H), 2.72-2.61 (m, 1H), 2.54 (s, 3H), 2.41-2.11 (m, 5H), 1.98 (br d, J=6.0 Hz, 1H), 1.79 (br d, J=8.0 Hz, 1H), 1.39 (s, 9H), 0.79-0.50 (m, 3H), 0.36-0.20 (m, 1H).

### Step 4:N-[(1-aminocyclopropyl)methyl]-1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine

To a solution of tert-butyl N-[1-[[[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]amino]methyl]cyclopropyl]carbamate (40 mg, 67.94 μmol, 1 eq) in DCM (1 mL) was added TsOH·H₂O (38.77 mg, 203.83 μmol, 3 eq). The mixture was stirred at 25° C. for 1 h. LCMS showed starting material was consumed and the desired mass was detected. The reaction was filtered and the filtered concentrated under reduced pressure to give compound N-[(1-aminocyclopropyl)methyl]-1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine (crude) as a white solid.

LC-MS [ESI, M+1]: 489.2.

### Step 5: 6-[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]-4,6-diazaspiro[2.4]heptan-5-one

To a solution of N-[(1-aminocyclopropyl)methyl]-1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-amine (30 mg, 61.40 μmol, 1 eq) in DCM (1 mL) was added CDI (29.87 mg, 184.20 μmol, 3 eq). The mixture was stirred at 80° C. for 2 h. LCMS showed starting material was consumed and the desired mass was detected. The reaction was filtered and the filtered concentrated under reduced pressure to give compound 6-[1-[6-[7-(3-fluorophenyl)-2-azaspiro[3.3]heptan-2-yl]-8-methyl-[1,2,4]triazolo[1,5-a]pyridin-2-yl]-2-oxabicyclo[2.1.1]hexan-5-yl]-4,6-diazaspiro[2.4]heptan-5-one (3.16 mg, 9.99% yield, 99.851% purity) as a yellow solid.

LC-MS [ESI, M+1]: 515.3

¹H NMR (400 MHz, CHLOROFORM-d) δ=7.43 (d, J=1.6 Hz, 1H), 7.35-7.27 (m, 1H), 7.05-6.86 (m, 3H), 6.62 (d, J=0.8 Hz, 1H), 4.10-4.04 (m, 1H), 4.04-3.95 (m, 3H), 3.92-3.84 (m, 2H), 3.64-3.51 (m, 4H), 3.50-3.45 (m, 1H), 3.39 (t, J=3.2 Hz, 1H), 2.54 (s, 3H), 2.38-2.15 (m, 4H), 2.07 (dd, J=2.8, 8.0 Hz, 1H), 1.90 (d, J=8.0 Hz, 1H), 0.74-0.59 (m, 3H), 0.56-0.47 (m, 1H).

| Compound | | |
|---|---|---|
| Number | Synthesis | LC/MS |
| 512 | Example 62 | m/z 515.3[M + H]⁺ |

### Example 63

### 6-[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]-4,6-diazaspiro[2.4]heptan-5-one (Compound 513)

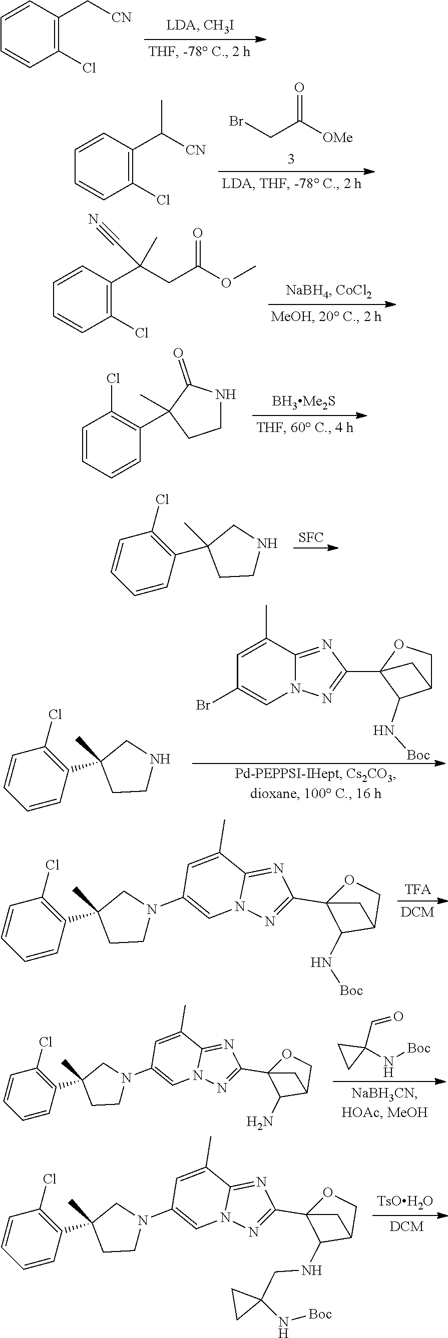

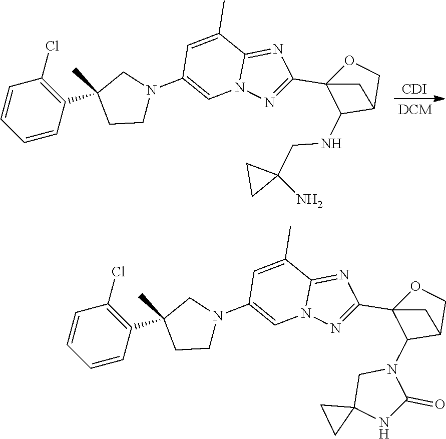

### Step 1: 2-(2-chlorophenyl)propanenitrile

To a solution of 2-(2-chlorophenyl)acetonitrile (10 g, 65.97 mmol, 1 eq) in THF (120 mL) was added dropwise LDA (2 M, 42.88 mL, 1.3 eq) at −60° C. over 0.1 h. After addition, the mixture was stirred at this temperature for 0.35 h, and CH₃I (12.17 g, 85.76 mmol, 5.34 mL, 1.3 eq) was added dropwise at −60° C. The resulting mixture was stirred at −60° C. for 1 h. TLC (SiO₂, PE:EtOAc=20:1, Rf=0.45) indicated reactant was consumed and many new spots formed. The cold reaction mixture was poured into NH₄Cl (80 ml). The reaction mixture was extracted with EtOAc (80 mL×3), the organic phase was dried, filtered, and concentrated under pressure to give the product. The residue was purified by flash silica gel chromatography (SiO₂, PE:EtOAc=20:1, Rf=0.45) to give compound 2-(2-chlorophenyl)propanenitrile (10.5 g, 96.11% yield) as a yellow liquid.

¹H NMR (400 MHz, CHLOROFORM-d) δ 7.59 (d, J=7.6 Hz, 1H), 7.41 (d, J=7.6 Hz, 1H), 7.37-7.32 (m, 1H), 7.32-7.26 (m, 1H), 4.36 (q, J=7.2 Hz, 1H), 1.64 (d, J=7.2 Hz, 3H).

### Step 2: methyl 3-(2-chlorophenyl)-3-cyano-butanoate

To a solution of 2-(2-chlorophenyl)propanenitrile (10.5 g, 63.40 mmol, 1 eq) in THF (250 mL) was added LDA (2 M, 38.04 mL, 1.2 eq) at −65° C. The mixture was stirred at −65° C. for 1 h, then methyl 2-bromoacetate (10.67 g, 69.74 mmol, 6.60 mL, 1.1 eq) was added to the solution at −65° C., then the mixture was stirred at 25° C. for 1 h. TLC (SiO₂, PE:EtOAc=3:1, Rf=0.35) indicated reactant was consumed and many new spot formed. The cold reaction mixture was poured into NH₄Cl (100 ml). The reaction mixture was extracted with EtOAc (100 mL×3), the organic phase was dried, filtered, and concentrated under pressure to give the product. The reaction was filtered and the filtered concentrated under reduced pressure to give a residue. The residue was purified by flash silica gel chromatography (SiO₂, PE:EtOAc=3:1, Rf=0.35) to give compound methyl 3-(2-chlorophenyl)-3-cyano-butanoate (15 g, 99.54% yield) as a yellow liquid.

1H NMR (400 MHz, CHLOROFORM-d) δ=7.69-7.62 (m, 1H), 7.46-7.39 (m, 1H), 7.35-7.27 (m, 2H), 3.63 (s, 3H), 3.47 (d, J=16.0 Hz, 1H), 3.16 (d, J=16.0 Hz, 1H), 2.02 (s, 3H).

### Step 3: 4-(2-chlorophenyl)-4-methyl-pyrrolidin-2-one

To a solution of methyl 3-(2-chlorophenyl)-3-cyano-butanoate (5 g, 21.04 mmol, 1 eq) in MeOH (50 mL) and H₂O (5 mL), then was added NaBH₄ (3.18 g, 84.15 mmol, 4 eq) and CoCl₂·6H₂O (20.02 g, 84.15 mmol, 4 eq) at 0° C. The mixture was stirred at 0-25° C. for 12 h. TLC (SiO₂, PE:EtOAc=0:1, Rf=0.4) indicated reactant was not consumed completely and some new spots formed. The cold reaction mixture was poured into H₂O (100 ml). The reaction solution was filtered and the filtrate was concentrated under the reduced pressure to give the residue. Then was added into K₂CO₃ (50 mL) and then extracted with DCM (50 mL×3), the organic phase was dried, filtered, and concentrated under pressure to give the product. The residue was purified by flash silica gel chromatography (SiO₂, PE:EtOAc=0:1, Rf=0.4) to give compound 4-(2-chlorophenyl)-4-methyl-pyrrolidin-2-one (1.4 g, 31.42% yield) as a yellow solid.

LC-MS [ESI, M+1]: 210.1.

¹H NMR (400 MHz, DMSO-d₆) δ=7.78 (s, 1H), 7.45-7.40 (m, 1H), 7.34-7.30 (m, 2H), 7.30-7.24 (m, 1H), 3.63-3.55 (m, 2H), 2.73 (d, J=16.0 Hz, 1H), 2.43 (d, J=16.0 Hz, 1H), 1.42 (s, 3H).

### Step 4: 3-(2-chlorophenyl)-3-methyl-pyrrolidine

A mixture of 4-(2-chlorophenyl)-4-methyl-pyrrolidin-2-one (1.8 g, 8.58 mmol, 1 eq) in THF (20 mL) was degassed and purged with N₂ for 3 times, and then BH₃·Me₂S (10 M, 3.43 mL, 4 eq) was added dropwise at 0° C. After addition, the mixture was stirred at this temperature for 0.5 h, and then the mixture was stirred at 60° C. for 2 h under N₂ atmosphere. TLC (SiO₂, EtOAc:NH₃·MeOH (7 M)=3:1, Rf=0.1) indicated reactant was consumed completely and many new spots formed. The reaction was quenched by addition of 10 mL of methanol at 0° C. under N₂ atmosphere, and then the mixture was stirred at 60° C. for 2 h under N₂ atmosphere. The reaction solution was concentrated under the reduced pressure to give the residue. The reaction mixture was quenched by water (50 mL) and extracted with EtOAc (50 mL×3), the organic phase was dried, filtered, and concentrated under pressure to give the product. The residue was purified by flash silica gel chromatography (SiO₂, EtOAc:NH 3·MeOH (7 M)=3:1, Rf=0.1) to give compound 3-(2-chlorophenyl)-3-methyl-pyrrolidine (750 mg, 3.72 mmol, 43.30% yield, 97% purity) as a yellow oil.

LC-MS [ESI, M+1]: 196.1.

¹H NMR (400 MHz, CHLOROFORM-d) δ=7.36 (d, J=7.6 Hz, 1H), 7.29 (dd, J=1.6, 7.6 Hz, 1H), 7.24-7.18 (m, 1H), 7.18-7.12 (m, 1H), 3.35 (d, J=11.2 Hz, 1H), 3.22 (d, J=11.2 Hz, 1H), 3.19-3.11 (m, 1H), 3.10-3.03 (m, 1H), 2.21-2.16 (m, 2H), 1.42 (s, 3H).

### Step 5: (3R)-3-(2-chlorophenyl)-3-methyl-pyrrolidine

The 3-(2-chlorophenyl)-3-methyl-pyrrolidine (600 mg, 3.07 mmol, 1 eq) was purified by SFC. The residue was purified by prep-HPLC (column: DAICEL CHIRALPAK IG (250 mm*30 mm, 10 um);mobile phase: [Hexane-EtOH (0.1% IPAm)]; B %: 10%, isocratic elution mode, peak 1) to give compound (3R)-3-(2-chlorophenyl)-3-methyl-pyrrolidine (290 mg, 45.80% yield) as a yellow oil.

### Step 6: tert-butyl N-[1-[6-[(3R)-3-(2-chlorophenyl)-3-methyl-pyrrolidin-1-yl]-8 methyl-[1,2,4]triazolo[1,5-a]pyridin-2-yl]-2-oxabicyclo[2.1.1]hexan-5-yl]carbamate

A mixture of (3R)-3-(2-chlorophenyl)-3-methyl-pyrrolidine (100 mg, 511.02 μmol, 1 eq), tert-butyl N-[1-(6-bromo-8-methyl-[1,2,4]triazolo[1,5-a]pyridin-2-yl)-2-oxabicyclo[2.1.1]hexan-5-yl]carbamate (209.15 mg, 511.02 μmol, 1 eq), Cs₂CO₃ (499.50 mg, 1.53 mmol, 3 eq), RuPhos (23.85 mg, 51.10 μmol, 0.1 eq) and Pd₂(dba)₃ (46.79 mg, 51.10 μmol, 0.1 eq) in dioxane (2 mL) was degassed and purged with N₂ for 3 times, and then the mixture was stirred at 100° C. for 1 h under N₂ atmosphere. LCMS showed starting material was consumed and one main peak with desired mass was detected. The mixture was filtered, and concentrated under pressure to give the product. The residue was purified by prep-HPLC (column: Phenomenex Luna C18 40 g; mobile phase: [water(FA)—ACN]; B %: 50%-63%, 8 min.) to give compound tert-butyl N-[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]carbamate (70 mg, 24.05% yield) as a yellow gum.

LC-MS [ESI, M+1]: 524.2.

¹H NMR (400 MHz, DMSO-d₆) δ=7.87 (s, 1H), 7.51-7.43 (m, 2H), 7.38-7.32 (m, 1H), 7.32-7.27 (m, 1H), 7.21 (s, 1H), 6.63 (d, J=8.0 Hz, 1H), 3.93-3.87 (m, 2H), 3.84 (d, J=9.6 Hz, 1H), 3.74 (d, J=6.0 Hz, 1H), 3.54 (d, J=9.6 Hz, 2H), 3.48 (s, 1H), 2.91 (s, 2H), 2.54 (s, 3H), 2.41-2.33 (m, 1H), 1.91 (d, J=5.6 Hz, 1H), 1.59 (d, J=8.0 Hz, 1H), 1.51-1.25 (m, 12H).

### Step 7: 1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine

To a solution of tert-butyl N-[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]carbamate (60 mg, 114.49 μmol, 1 eq) in DCM (1 mL) was added TFA (39.16 mg, 343.48 μmol, 25.51 μL, 3 eq). The mixture was stirred at 25° C. for 0.5 h. LCMS showed starting material was consumed and one main peak with desired mass was detected. The mixture was concentrated under pressure to give compound 1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine (48 mg, crude) as a yellow gum.

LC-MS [ESI, M+1]: 424.2.

¹H NMR (400 MHz, DMSO-d₆) δ=7.86 (s, 1H), 7.52-7.43 (m, 2H), 7.39-7.25 (m, 2H), 7.20 (s, 1H), 3.84 (d, J=9.6 Hz, 1H), 3.80-3.75 (m, 1H), 3.74-3.69 (m, 1H), 3.54 (d, J=9.6 Hz, 1H), 3.51-3.43 (m, 1H), 3.41-3.36 (m, 1H), 3.14 (d, J=2.8 Hz, 1H), 2.67 (t, J=2.8 Hz, 1H), 2.54 (s, 4H), 2.42-2.31 (m, 1H), 1.90-1.66 (m, 3H), 1.50 (d, J=8.0 Hz, 1H), 1.41 (s, 3H).

### Step 8: tert-butyl N-[1-[[[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]amino]methyl]cyclopropyl]carbamate

To a solution of 1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine (48 mg, 113.22 μmol, 1 eq) and tert-butyl N-(1-formylcyclopropyl)carbamate (41.94 mg, 226.45 μmol, 2 eq) in MeOH (2 mL) was added NaBH₃CN (21.35 mg, 339.67 μmol, 3 eq) at 0° C. over 0.1 h. The resulting mixture was stirred at 0° C. for 0.9 h. LCMS showed starting material was consumed and one main peak with desired mass was detected. The mixture was filtered, and concentrated under pressure to give the product. The residue was purified by prep-TLC (SiO₂, EtOAc:NH₃·MeOH (7M)=20:1, Rf=0.5) to give compound tert-butyl N-[1-[[[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]amino]methyl]cyclopropyl]carbamate (35 mg, 50.55% yield) was obtained as a yellow gum.

LC-MS [ESI, M+1]: 593.5.

### Step 9: N-[(1-aminocyclopropyl)methyl]-1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine

To a solution of tert-butyl N-[1-[[[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]amino]methyl]cyclopropyl]carbamate (35 mg, 59.01 μmol, 1 eq) in DCM (1.5 mL) was added TsOH (30.48 mg, 177.02 μmol, 3 eq). The mixture was stirred at 25° C. for 3 h. LCMS showed starting material was consumed and one main peak with desired mass was detected. The mixture was adjusted to pH=9 with solid NaHCO₃. The reaction mixture was quenched by H₂O (2 mL) and extracted with EtOAc (2 mL×3), the organic phase was dried, filtered, and concentrated under pressure to give compound N-[(1-aminocyclopropyl)methyl]-1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine (29 mg, crude) was obtained as a yellow gum.

LC-MS [ESI, M+1]: 493.2

### Step 10: 6-[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]-4,6-diazaspiro[2.4]heptan-5-one

To a solution of N-[(1-aminocyclopropyl)methyl]-1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-amine (29.00 mg, 58.82 μmol, 1 eq) in DCM (3 mL) was added CDI (76.30 mg, 470.55 μmol, 8 eq). The mixture was stirred at 45° C. for 3 h. LCMS showed starting material was consumed and one main peak with desired mass was detected. The mixture was filtered, and concentrated under pressure to give the product. The residue was purified by prep-HPLC (column: CD04-Welch Utimate C18 150*25*7 um; mobile phase: [water(FA)—ACN]; gradient:35%-65% B over 15 min.) to give the product compound 6-[1-[6-[(3R)-3-(2-chlorophenyl)-3-methyl-pyrrolidin-1-yl]-8-methyl-[1,2,4]triazolo[1,5-a]pyridin-2-yl]-2-oxabicyclo[2.1.1]hexan-5-yl]-4,6-diazaspiro[2.4]heptan-5-one (23.92 mg, 78.13% yield) as a white solid.

LC-MS [ESI, M+1]: 519.4.

¹H NMR (400 MHz, CHLOROFORM-d) δ 7.67 (d, J=1.6 Hz, 1H), 7.42 (dd, J=1.2, 8.0 Hz, 1H), 7.36-7.30 (m, 1H), 7.27 (s, 2H), 7.24-7.18 (m, 1H), 6.95 (s, 1H), 4.15-4.05 (m, 2H), 4.05-3.98 (m, 2H), 3.91 (d, J=9.2 Hz, 1H), 3.59 (dd, J=4.4, 8.8 Hz, 2H), 3.52 (dd, J=2.0, 9.2 Hz, 1H), 3.50-3.43 (m, 1H), 3.41 (t, J=2.8 Hz, 1H), 2.63 (s, 3H), 2.57-2.41 (m, 2H), 2.10 (dd, J=2.8, 8.0 Hz, 1H), 1.93 (d, J=8.4 Hz, 1H), 1.49 (s, 3H), 0.79-0.61 (m, 3H), 0.61-0.49 (m, 1H).

### Example 64

### 1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine (Compound 514)

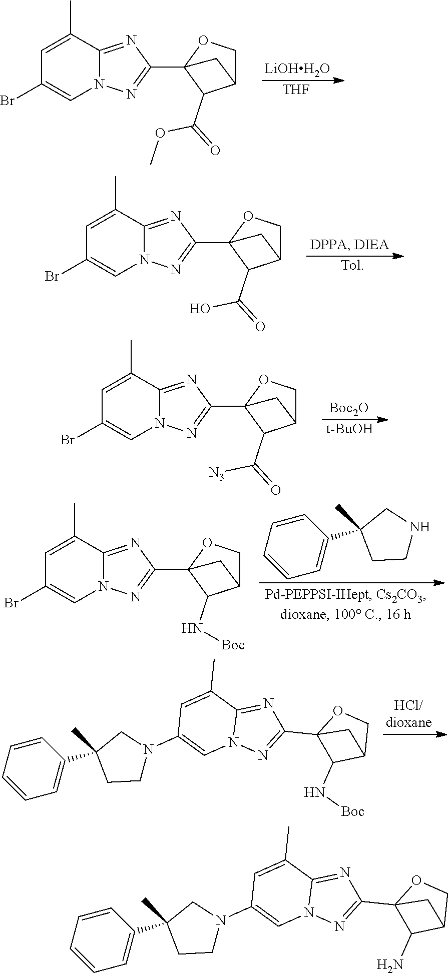

### Step 1: 1-(6-bromo-8-methyl-[1,2,4]triazolo[1,5-a]pyridin-2-yl)-2-oxabicyclo[2.1.1]hexane-5-carboxylic acid

To a solution of methyl 1-(6-bromo-8-methyl-[1,2,4]triazolo[1,5-a]pyridin-2-yl)-2-oxabicyclo[2.1.1]hexane-5-carboxylate (4 g, 11.36 mmol, 1 eq) in THF (30 mL) and H₂O (10 mL) was added LiOH·H₂O (1.43 g, 34.07 mmol, 3 eq). The mixture was stirred at 25° C. for 12 h. LCMS showed starting material was consumed and the desired mass was detected. The pH was adjusted to around 4 by aqueous. FA, and then extracted with ethyl acetate (50 mL×2). The combined organic layers were washed with brine (50 mL), dried over Na₂SO₄, filtered and concentrated under reduced pressure to give compound 1-(6-bromo-8-methyl-[1,2,4]triazolo[1,5-a]pyridin-2-yl)-2-oxabicyclo[2.1.1]hexane-5-carboxylic acid (3.7 g, 10.83 mmol, 95.37% yield, 99% purity) as yellow solid.

LC-MS [ESI, M+1]:339.9.

¹H NMR (400 MHz, METHANOL-d4) δ=8.87 (s, 1H), 7.64 (s, 1H), 4.23 (d, J=6.0 Hz, 1H), 3.99 (d, J=6.0 Hz, 1H), 3.37 (d, J=2.8 Hz, 1H), 3.30 (br s, 1H), 2.62 (s, 3H), 2.29 (dd, J=2.8, 7.2 Hz, 1H), 1.81 (d, J=7.6 Hz, 1H)

### Step 2: 1-(6-bromo-8-methyl-[1,2,4]triazolo[1,5-a]pyridin-2-yl)-2-oxabicyclo[2.1.1]hexane-5-carbonyl azide

To a solution of 1-(6-bromo-8-methyl-[1,2,4]triazolo[1,5-a]pyridin-2-yl)-2-oxabicyclo[2.1.1]hexane-5-carboxylic acid (2.5 g, 7.39 mmol, 1 eq) in Tol. (25 mL) was added DPPA (2.24 g, 8.13 mmol, 1.76 mL, 1.1 eq) and DIEA (1.05 g, 8.13 mmol, 1.42 mL, 1.1 eq). The mixture was stirred at 25° C. for 2 h. LCMS showed starting material was consumed and the desired mass was detected. The mixture was quenched with water (50 mL) and extracted with ethyl acetate (80 mL×2). The combined organic phase was washed with brine (50 mL), dried with anhydrous Na₂SO₄, filtered and concentrated in vacuum to give compound 1-(6-bromo-8-methyl-[1,2,4]triazolo[1,5-a]pyridin-2-yl)-2-oxabicyclo[2.1.1]hexane-5-carbonyl azide (2.8 g, crude) as yellow oil.

LC-MS [ESI, M-28+1]:336.9.

### Step 3: tert-butyl (1-(6-bromo-8-methyl-[1,2,4]triazolo[1,5-a]pyridin-2-yl)-2-oxabicyclo[2.1.1]hexan-5-yl)carbamate

To a solution of 1-(6-bromo-8-methyl-[1,2,4]triazolo[1,5-a]pyridin-2-yl)-2-oxabicyclo[2.1.1]hexane-5-carbonyl azide (2.8 g, 7.71 mmol, 1 eq) in t-BuOH (30 mL) was added Boc₂O (3.37 g, 15.42 mmol, 3.54 mL, 2 eq).The mixture was stirred at 90° C. for 1 h. LCMS showed starting material was consumed and the desired mass was detected. The reaction mixture was extracted with ethyl acetate (80 mL×2). The combined organic phase was washed with brine (50 mL), dried with anhydrous Na₂SO₄, filtered and concentrated in vacuum. The residue was purified by column chromatography (SiO₂, PE:EtOAc=1:1, Rf=0.4) to give compound tert-butyl N-[1-(6-bromo-8-methyl-[1,2,4]triazolo[1,5-a]pyridin-2-yl)-2-oxabicyclo[2.1.1]hexan-5-yl]carbamate (1.7 g, 3.99 mmol, 51.72% yield, 96% purity) as yellow solid.

LC-MS [ESI, M+1]:410.9.

¹H NMR (400 MHz, CHLOROFORM-d) δ=8.54 (d, J=0.8 Hz, 1H), 7.39 (s, 1H), 5.22 (br d, J=6.8 Hz, 1H), 4.22-4.11 (m, 1H), 4.00 (d, J=6.4 Hz, 1H), 3.90 (br d, J=6.4 Hz, 1H), 3.11 (br s, 1H), 2.65 (s, 3H), 2.13 (br dd, J=2.8, 8.4 Hz, 1H), 1.84 (d, J=8.4 Hz, 1H), 1.41 (s, 9H).

### Step 4: tert-butyl (1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamate

A mixture of tert-butyl N-[1-(6-bromo-8-methyl-[1,2,4]triazolo[1,5-a]pyridin-2-yl)-2-oxabicyclo[2.1.1]hexan-5-yl]carbamate (1.7 g, 4.15 mmol, 1 eq), (3R)-3-methyl-3-phenyl-pyrrolidine (736.72 mg, 4.57 mmol, 1.1 eq), 1,3-bis[2,6-bis(1-propylbutyl)phenyl]-4,5-dichloro-2H-imidazol-1-ium-2-ide;3-chloropyridine;dichloropalladium (202.03 mg, 207.68 μmol, 0.05 eq) and Cs₂CO₃ (4.06 g, 12.46 mmol, 3 eq) in dioxane (25 mL) was degassed and purged with N₂ for 3 times, and then the mixture was stirred at 105° C. for 12 h under N₂ atmosphere. LCMS showed starting material was consumed and the desired mass was detected. T The mixture was quenched with water (10 mL) and extracted with ethyl acetate (10 mL×2). The combined organic phase was washed with brine (15 mL), dried with anhydrous Na₂SO₄, filtered and concentrated in vacuum. The residue was purified by column chromatography (SiO₂, PE:EtOAc=1:1, Rf=0.6) to give compound tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (1.6 g, 2.68 mmol, 64.51% yield, 82% purity) as yellow solid.

LC-MS [ESI, M+1]:490.2.

¹H NMR (400 MHz, CHLOROFORM-d) δ=7.65 (d, J=1.6 Hz, 1H), 7.43-7.35 (m, 2H), 7.35-7.31 (m, 2H), 7.28 (br d, J=2.0 Hz, 1H), 6.95 (s, 1H), 4.25-4.13 (m, 1H), 4.00 (d, J=6.8 Hz, 1H), 3.89 (br d, J=6.4 Hz, 1H), 3.61 (d, J=8.8 Hz, 1H), 3.57-3.45 (m, 2H), 3.39 (dt, J=3.6, 8.8 Hz, 1H), 3.12 (br s, 1H), 2.65 (s, 3H), 2.38 (td, J=8.4, 12.4 Hz, 1H), 2.31-2.22 (m, 1H), 2.13 (br d, J=6.0 Hz, 1H), 1.87 (d, J=8.4 Hz, 1H), 1.48 (s, 3H), 1.43 (s, 9H).

### Step 5: 1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine

To a solution of tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (1.5 g, 3.06 mmol, 1 eq) in DCM (10 mL) was added TFA (7.68 g, 67.31 mmol, 5.00 mL, 21.97 eq). The mixture was stirred at 25° C. for 0.5 h. LCMS showed starting material was consumed and the desired mass was detected. The pH was adjusted to around 7 by progressively aq. NaHCO₃, and then extracted with ethyl acetate (100 mL×2). The combined organic layers were washed with brine (200 mL), dried over Na₂SO₄, filtered and concentrated under reduced pressure to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (1.24 g, crude) as yellow solid.

LC-MS [ESI, M+1]:390.1.

¹H NMR (400 MHz, CHLOROFORM-d) δ=7.56 (d, J=1.2 Hz, 1H), 7.33-7.26 (m, 2H), 7.26-7.21 (m, 2H), 7.18 (br s, 1H), 6.85 (s, 1H), 3.95-3.83 (m, 2H), 3.51 (d, J=8.8 Hz, 1H), 3.47-3.34 (m, 3H), 3.29 (dt, J=3.6, 8.8 Hz, 1H), 2.81 (t, J=3.2 Hz, 1H), 2.55 (s, 3H), 2.29 (td, J=8.4, 12.0 Hz, 1H), 2.22-2.09 (m, 1H), 1.98 (s, 1H), 1.88 (br dd, J=2.8, 8.0 Hz, 2H), 1.74-1.68 (m, 1H), 1.38 (s, 3H).

### Example 65

### 7-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-2-oxa-5,7-diazaspiro[3.4]octan-6-one

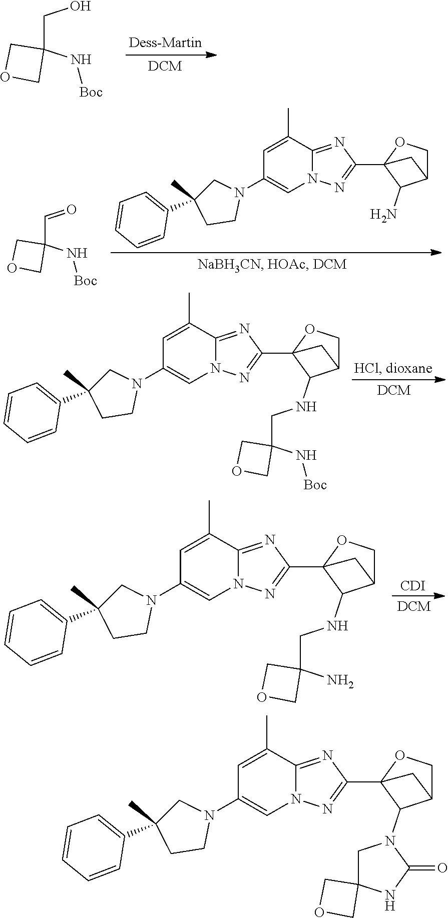

### Step 1: tert-butyl N-(3-formyloxetan-3-yl)carbamate

To a solution of tert-butyl N-[3-(hydroxymethyl)oxetan-3-yl]carbamate (500 mg, 2.46 mmol, 1 eq) in DCM (5 mL) under nitrogen was added DMP (1.04 g, 2.46 mmol, 762.22 μL, 1 eq) at 0° C. The mixture was stirred at 0° C. for 20 min. TLC (PE:EtOAc=1:1, Rf=0.31) showed the starting material was consumed completely and one new spot formed. The mixture was filtered and concentrated to give a residue. The residue was purified by column chromatography (SiO₂, PE:EtOAc=1:1, Rf=0.31) to give compound tert-butyl N-(3-formyloxetan-3-yl) carbamate (500 mg, 81% yield) as colorless oil.

¹H NMR (400 MHz, CHLOROFORM-d) δ=9.81 (br s, 1H), 4.98-4.51 (m, 5H), 1.45 (s, 9H).

### Step 2: tert-butyl N-[3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]oxetan-3-yl]carbamate

To a solution of 1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-5-amine (150 mg, 385.12 μmol, 1 eq) in DCM (3 mL) was added DIEA (99.55 mg, 770.23 μmol, 134.16 μL, 2 eq) and AcOH (23.13 mg, 385.12 μmol, 22.05 μL, 1 eq) and then tert-butyl N-(3-formyloxetan-3-yl)carbamate (232.48 mg, 1.16 mmol, 3 eq), NaBH₃CN (72.60 mg, 1.16 mmol, 3 eq). The mixture was stirred at 25° C. for 10 min. LCMS showed starting material was consumed and the desired mass was detected. The reaction was filtered and the filtered concentrated under reduced pressure to give a residue. The residue was purified by column chromatography (SiO₂, PE:EtOAc=10:1, Rf=0.23) to give compound tert-butyl N-[3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]oxetan-3-yl]carbamate (100 mg, 45% yield) as colorless oil.

LC-MS [ESI, M+1]: 575.4.

### Step 3: N-[(3-aminooxetan-3-yl)methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (Compound 510)

To a solution of tert-butyl N-[3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]oxetan-3-yl]carbamate (90 mg, 156.60 μmol, 1 eq) in DCM (1 mL) was added TFA (0.5 mL). The mixture was stirred at 25° C. for 10 min. LCMS showed starting material was consumed and the desired mass was detected. The reaction was added NH₃·H₂O (0.5 mL) and concentrated to give a residue. The crude product was purified by reversed-phase HPLC (column: CD07-Daisogel SP-100-8-ODS-PK 150*25*10 um; mobile phase: [water(NH₄HCO₃)—ACN]; gradient:22%-52% B over 10 min) to give compound N-[(3-aminooxetan-3-yl)methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (40 mg, 53% yield) as white solid.

LC-MS [ESI, M+1]: 475.7.

¹H NMR (400 MHz, CHLOROFORM-d) δ=7.64 (d, J=1.6 Hz, 1H), 7.40-7.34 (m, 2H), 7.33-7.29 (m, 2H), 7.27-7.23 (m, 1H), 6.94 (d, J=1.2 Hz, 1H), 4.48-4.40 (m, 3H), 4.38-4.34 (m, 1H), 3.98-3.93 (m, 1H), 3.93-3.88 (m, 1H), 3.59 (d, J=8.8 Hz, 1H), 3.55-3.45 (m, 2H), 3.39 (dt, J=3.6, 8.8 Hz, 1H), 3.27 (d, J=2.8 Hz, 1H), 3.09-3.01 (m, 1H), 2.97-2.91 (m, 2H), 2.63 (s, 3H), 2.37 (td, J=8.4, 12.0 Hz, 1H), 2.30-2.20 (m, 1H), 1.98 (br d, J=3.2 Hz, 1H), 1.96 (br d, J=3.2 Hz, 1H), 1.77 (d, J=8.4 Hz, 1H), 1.46 (s, 3H)

### Step 4: 7-[1-[8-methyl-6-[(3R)-3-methyl-3-phenyl-pyrrolidin-1-yl]-[1,2,4]triazolo [1,5-a]pyridin-2-yl]-2-oxabicyclo[2.1.1]hexan-5-yl]-2-oxa-5,7-diazaspiro[3.4]octan-6-one (Compound 509)

To a solution of N-[(3-aminooxetan-3-yl)methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (20 mg, 42.14 μmol, 1 eq) in ACN (1 mL) was added CDI (34.17 mg, 210.71 μmol, 5 eq). The mixture was stirred at 50° C. for 0.5 h. LCMS showed starting material was consumed and the desired mass was detected. The reaction was concentrated under reduced pressure to give a residue. The crude product was purified by prep-HPLC (column: CD07-Daisogel SP-100-8-ODS-PK 150*25*10 um; mobile phase: [water(NH₄HCO₃)—ACN]; gradient:25%-55% B over 10 min) to give compound 7-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxa-5,7-diazaspiro octan-6-one (12.5 mg, 58% yield) as white solid.

LC-MS [ESI, M+1]: 501.3.

¹H NMR (400 MHz, CHLOROFORM-d) δ=7.65 (d, J=1.6 Hz, 1H), 7.40-7.35 (m, 2H), 7.34-7.30 (m, 2H), 7.25 (br s, 1H), 6.98 (s, 1H), 4.96 (s, 1H), 4.73-4.64 (m, 3H), 4.54 (d, J=7.2 Hz, 1H), 4.05-3.99 (m, 2H), 3.98-3.94 (m, 1H), 3.91 (br s, 1H), 3.74 (dd, J=1.6, 10.4 Hz, 1H), 3.61 (d, J=8.8 Hz, 1H), 3.57-3.47 (m, 2H), 3.44-3.37 (m, 2H), 2.65 (s, 3H), 2.39 (td, J=8.0, 12.0 Hz, 1H), 2.31-2.22 (m, 1H), 2.11 (br d, J=7.2 Hz, 1H), 1.96 (d, J=8.4 Hz, 1H), 1.47 (s, 3H).

| Compound | | |
|---|---|---|
| Number | Synthesis | LC/MS |
| 515 | Similar to Example 65 | m/z 500.2 [M + H]⁺ |
| 516 | Similar to Example 65 | m/z 621.3 [M + H]⁺ |
| 517 | Similar to Example 65 | m/z 642.5 [M + H]⁺ |
| 518 | Similar to Example 65 | m/z 644.4 [M + H]⁺ |
| 519 | Similar to Example 65 | m/z 621.4 [M + H]⁺ |
| 520 | Similar to Example 65 | m/z 621.4 [M + H]⁺ |
| 521 | Similar to Example 65 | m/z 503.3 [M + H]⁺ |
| 522 | Similar to Example 65 | m/z 529.5 [M + H]⁺ |
| 523 | Similar to Example 65 | m/z 485.2 [M + H]⁺ |
| 524 | Similar to Example 65 | m/z 485.4 [M + H]⁺ |
| 525 | Similar to Example 65 | m/z 571.3 [M + H]⁺ |
| 526 | Similar to Example 65 | m/z 571.3 [M + H]⁺ |

### Example 66

### 4-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)morpholine (Compound 527)

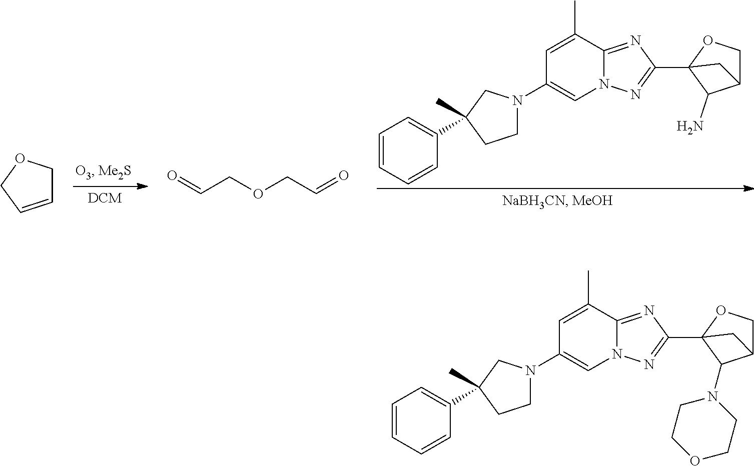

### Step 1: 2,2′-oxydiacetaldehyde 2,2′-oxydiacetaldehyde

A stream of OZONE (1.37 g, 28.53 mmol, 1 eq) is passed through a cooled −78° C. solution of 2,5-dihydrofuran (2 g, 28.53 mmol, 2.16 mL, 1 eq) in DCM (100 mL) until TLC analysis (PE:EtOAc=1:3) indicates that conversion of starting material (Rf=0.10) to a more polar product is complete. The reaction mixture is then quenched with Me₂S (4.43 g, 71.34 mmol, 5.24 mL, 2.5 eq) stirred at −20° C. and stirred at 25° C. for 1 h. The reaction was monitored by TLC, TLC (PE:EtOAc=1:1) indicated reactant was consumed completely and one new spot formed. The reaction mixture was concentrated under the reduced pressure to give compound 2-(2-oxoethoxy)acetaldehyde (2 g, 19.59 mmol, 68.66% yield) as yellow oil.

¹H NMR (400 MHz, CHLOROFORM-d) δ=9.74 (s, 2H), 5.72 (s, 2H), 4.31-4.25 (m, 4H), 4.23 (br s, 4H), 2.65 (s, 59H)

### Step 2: 4-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)morpholine

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (30 mg, 77.02 μmol, 1 eq) in MeOH (0.5 mL) was added 2-(2-oxoethoxy)acetaldehyde (78.63 mg, 770.23 μmol, 10 eq) and NaBH₃CN (9.68 mg, 154.05 μmol, 2 eq). The mixture was stirred at 0° C. for 0.5 h. LCMS showed starting material was consumed and the desired mass was detected. The reaction mixture was concentrated under the reduced pressure to give the crude product. The residue was purified by prep-HPLC (FA condition column: CD24-WePure Biotech XPT C18 150*25*7 um; mobile phase: [H2O (10 mM NH₄HCO₃)—ACN]; gradient:36%-66% B over 15.0 min) to give compound 4-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]morpholine (4.35 mg, 9.13 μmol, 11.86% yield, 96.478% purity) as gray solid.

LC-MS [ESI, M+1]:460.3.

¹H NMR (400 MHz, CHLOROFORM-d) δ=7.64 (d, J=1.6 Hz, 1H), 7.40-7.33 (m, 2H), 7.33-7.29 (m, 2H), 7.26 (br s, 1H), 6.93 (s, 1H), 4.18 (d, J=5.2 Hz, 1H), 3.91 (d, J=5.6 Hz, 1H), 3.75-3.65 (m, 2H), 3.64-3.56 (m, 3H), 3.54-3.43 (m, 2H), 3.38 (dt, J=3.6, 8.8 Hz, 1H), 2.97-2.91 (m, 1H), 2.76 (d, J=2.8 Hz, 1H), 2.62 (s, 3H), 2.42-2.20 (m, 6H), 2.00 (br dd, J=2.8, 8.0 Hz, 1H), 1.81 (d, J=8.0 Hz, 1H), 1.46 (s, 3H).

### Example 67

### 5,5-dimethyl-3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]oxazolidin-2-one

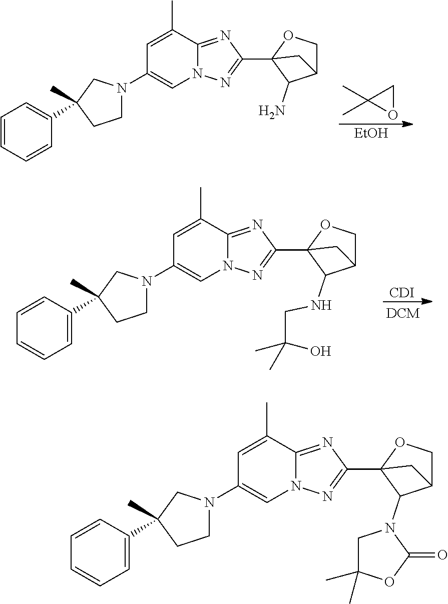

### Step 1: 2-methyl-1-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]propan-2-ol (Compound 528)

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (100 mg, 256.74 μmol, 1 eq) in EtOH (2 mL) was added 2,2-dimethyloxirane (37.03 mg, 513.49 μmol, 45.60 μL, 2 eq). The mixture was stirred at 60° C. for 2 h. LCMS showed starting material was consumed and the desired mass was detected. The mixture was concentrated to give compound 2-methyl-1-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]propan-2-ol (80 mg, 63% yield) as white solid.

LC-MS [ESI, M+1]: 462.2.

¹H NMR (400 MHz, CHLOROFORM-d) δ=7.63 (d, J=2.0 Hz, 1H), 7.40-7.34 (m, 2H), 7.34-7.29 (m, 2H), 7.27-7.23 (m, 1H), 6.95 (d, J=0.8 Hz, 1H), 4.01-3.92 (m, 2H), 3.59 (d, J=8.8 Hz, 1H), 3.55-3.44 (m, 2H), 3.39 (dt, J=3.6, 8.8 Hz, 1H), 3.30 (d, J=3.2 Hz, 1H), 2.95 (br s, 1H), 2.72-2.63 (m, 2H), 2.63 (s, 3H), 2.37 (td, J=8.4, 12.0 Hz, 1H), 2.30-2.21 (m, 1H), 1.92 (dd, J=3.2, 8.0 Hz, 1H), 1.80 (br d, J=8.4 Hz, 1H), 1.46 (s, 3H), 1.14 (d, J=7.2 Hz, 6H).

### Step 2: 5,5-dimethyl-3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]oxazolidin-2-one (Compound 529)

To a solution of 2-methyl-1-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]propan-2-ol (30 mg, 64.99 μmol, 1 eq) in ACN (3 mL) was added CDI (52.69 mg, 324.96 μmol, 5 eq). The mixture was stirred at 50° C. for 0.5 h. LCMS showed starting material was consumed and the desired mass was detected. The residue was purified by prep-HPLC purification (column: CD04-Welch Utimate C18 150*25*7 um; mobile phase: [water(FA)—ACN]; gradient:46%-76% B over 10 min) to give compound 5,5-dimethyl-3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]oxazolidin-2-one (7.03 mg, 22% yield) as white solid.

LC-MS [ESI, M+1]: 488.2.

¹H NMR (400 MHz, CHLOROFORM-d) δ=7.64 (s, 1H), 7.41-7.34 (m, 2H), 7.34-7.29 (m, 2H), 7.25 (br s, 1H), 6.96 (s, 1H), 4.06-4.02 (m, 1H), 4.01-3.96 (m, 2H), 3.60 (d, J=8.8 Hz, 1H), 3.56-3.50 (m, 2H), 3.50-3.44 (m, 2H), 3.40 (dt, J=3.6, 8.4 Hz, 1H), 3.29 (d, J=8.8 Hz, 1H), 2.63 (s, 3H), 2.38 (td, J=8.4, 12.0 Hz, 1H), 2.30-2.22 (m, 1H), 2.08 (dd, J=2.8, 8.4 Hz, 1H), 1.97 (d, J=8.4 Hz, 1H), 1.47 (s, 3H), 1.41 (s, 3H), 1.36 (s, 3H).

### Example 68

### 1-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)amino)cyclopropane-1-carbonitrile (Compound 530)

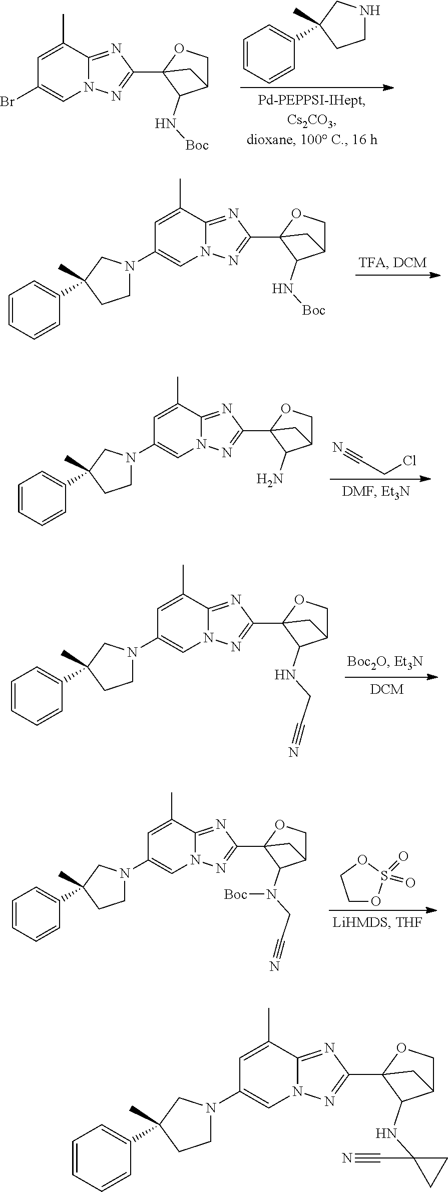

### Step 1: tert-butyl (1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamate

To a solution of (3R)-3-methyl-3-phenyl-pyrrolidine (354.57 mg, 2.20 mmol, 1 eq) and tert-butyl N-[1-(6-bromo-8-methyl-[1,2,4]triazolo[1,5-a]pyridin-2-yl)-2-oxabicyclo[2.1.1]hexan-5-yl]carbamate (900 mg, 2.20 mmol, 1 eq) in dioxane (12 mL) was added 1,3-bis[2,6-bis(1-propylbutyl)phenyl]-4,5-dichloro-2H-imidazol-1-ium-2-ide;3-chloropyridine;dichloropalladium (106.96 mg, 109.95 μmol, 0.05 eq) and Cs₂CO₃ (2.15 g, 6.60 mmol, 3 eq). The mixture was stirred at 100° C. for 12 h under N₂. LCMS showed starting material was consumed and one main peak with desired mass was detected. The reaction mixture was concentrated under the reduced pressure to give the product. The residue was purified by prep-HPLC (FA condition column: CD05-Phenomenex luna C18 150*40*10 um; mobile phase: [water(FA)—ACN]; gradient:53%-83% B over 11 min) to give compound tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (810 mg, 75% yield) as a yellow solid.

¹H NMR (400 MHz, CHLOROFORM-d) δ=7.65 (s, 1H), 7.41-7.34 (m, 2H), 7.33-7.29 (m, 2H), 7.27-7.22 (m, 1H), 6.96 (br s, 1H), 4.13 (br d, J=5.2 Hz, 1H), 3.98 (br d, J=6.4 Hz, 1H), 3.88 (br d, J=6.0 Hz, 1H), 3.59 (br d, J=8.8 Hz, 1H), 3.55-3.44 (m, 2H), 3.38 (dt, J=3.6, 8.4 Hz, 1H), 3.10 (br s, 1H), 2.64 (s, 3H), 2.42-2.32 (m, 1H), 2.26 (dt, J=3.6, 7.6 Hz, 1H), 2.15 (br d, J=6.4 Hz, 1H), 1.84 (d, J=8.4 Hz, 1H), 1.46 (s, 3H), 1.41 (s, 9H).

### Step 2: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine

To a solution of tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (810 mg, 1.65 mmol, 1 eq) in DCM (3 mL) was added TFA (2.30 g, 20.19 mmol, 1.5 mL, 12.21 eq). The mixture was stirred at 25° C. for 0.5 h. LCMS showed starting material was consumed and one main peak with desired mass was detected. The reaction mixture was concentrated under reduced pressure to give a residue. The pH was adjusted to around 7 by progressively NaHCO₃ (aq.), and then extracted with EtOAc (10 mL×3).The combined organic layers were washed with brine (5 mL), dried over Na₂SO₄, filtered and concentrated under reduced pressure to give compound 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (612 mg, 95% yield) as a yellow solid.

LC-MS [ESI, M+1]: 390.2.

### Step 3: 2-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)amino)acetonitrile

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (50 mg, 128.37 μmol, 1 eq) in DMF (0.5 mL) was added TEA (25.98 mg, 256.74 μmol, 35.74 μL, 2 eq) and 2-chloroacetonitrile (38.77 mg, 513.49 μmol, 32.50 μL, 4 eq). The mixture was stirred at 25° C. for 12 h. LCMS showed starting material was consumed and one main peak with desired mass was detected. The residue was purified by prep-TLC (SiO₂, PE:EtOAc=0:1, Rf=0.5) to give compound 2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]acetonitrile (30 mg, 55% yield) as a yellow gum.

LC-MS [ESI, M+1]: 429.2.

¹H NMR (400 MHz, CHLOROFORM-d) δ=7.59 (d, J=1.6 Hz, 1H), 7.36-7.29 (m, 2H), 7.27 (s, 3H), 6.89 (s, 1H), 3.99-3.94 (m, 1H), 3.92-3.87 (m, 1H), 3.65 (d, J=2.8 Hz, 2H), 3.54 (d, J=8.8 Hz, 1H), 3.49-3.38 (m, 2H), 3.37-3.28 (m, 2H), 3.08 (t, J=2.8 Hz, 1H), 2.57 (s, 3H), 2.35-2.27 (m, 1H), 2.22-2.17 (m, 1H), 2.01-1.97 (m, 1H), 1.82 (d, J=8.4 Hz, 1H), 1.41 (s, 3H).

### Step 4 tert-butyl (cyanomethyl)(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamate

To a solution of 2-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]acetonitrile (340 mg, 793.41 μmol, 1 eq) in DCM (4 mL) was added Boc₂O (346.32 mg, 1.59 mmol, 364.55 μL, 2 eq) and Et₃N (240.85 mg, 2.38 mmol, 331.30 μL, 3 eq). The mixture was stirred at 25° C. for 1 h. LCMS showed starting material was consumed and one main peak with desired mass was detected. The reaction mixture was concentrated under the reduced pressure to give the product. The reaction was added water (5 mL) and then extracted with ethyl acetate (10 mL×3).The combined organic phase was washed with brine (10 mL), dried with anhydrous Na₂SO₄, filtered and concentrated in vacuum. The residue was purified by prep-TLC (SiO₂, PE:EtOAc=1:1,Rf=0.3) to give compound tert-butyl N-(cyanomethyl)-N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (290 mg, 69% yield) as a yellow solid.

LC-MS [ESI, M+1]: 529.1

¹H NMR (400 MHz, CHLOROFORM-d) δ=7.63 (s, 1H), 7.41-7.34 (m, 2H), 7.33-7.29 (m, 2H), 7.24 (br s, 1H), 6.97 (s, 1H), 4.72-4.46 (m, 2H), 4.11-4.01 (m, 2H), 3.99-3.89 (m, 1H), 3.59 (br d, J=8.8 Hz, 1H), 3.53-3.46 (m, 2H), 3.44-3.20 (m, 2H), 2.63 (s, 3H), 2.45-2.32 (m, 1H), 2.31-2.13 (m, 2H), 2.07-1.97 (m, 1H), 1.56 (s, 9H), 1.46 (s, 3H).

### Step 5: 1-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)amino)cyclopropane-1-carbonitrile

To a solution of tert-butyl N-(cyanomethyl)-N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (200 mg, 378.33 μmol, 1 eq) and 1,3,2-dioxathiolane 2,2-dioxide (46.96 mg, 378.33 μmol, 1 eq) in THF (3 mL) was degassed and purged with N₂ for 3 times, was added LiHMDS (1 M, 1.89 mL, 5 eq) at 0° C., and then the mixture was stirred at 0° C. for 0.5 h under N₂ atmosphere. LCMS showed starting material was consumed and one main peak with desired mass was detected. The reaction was quenched by NH₄Cl (5 mL) slowly and then extracted with ethyl acetate (5 mL×3). The combined organic phase was washed with brine (5 mL), dried with anhydrous Na₂SO₄, filtered and concentrated in vacuum. The residue was purified by prep-HPLC (FA condition column: CD04-Welch Utimate C18 150*25*7 um; mobile phase: [water(FA)—ACN]; gradient:40%-70% B over 15 min) to give compound 1-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]cyclopropanecarbonitrile (42.43 mg, 25% yield) as a yellow solid.

LC-MS [ESI, M-11]: 455.2.

¹H NMR (400 MHz, CHLOROFORM-d) δ=7.63 (d, J=1.6 Hz, 1H), 7.40-7.33 (m, 2H), 7.33-7.28 (m, 2H), 7.27-7.22 (m, 1H), 6.92 (s, 1H), 4.07-3.89 (m, 2H), 3.58 (d, J=8.8 Hz, 1H), 3.52-3.43 (m, 3H), 3.37 (dt, J=3.6, 8.8 Hz, 1H), 3.22 (t, J=2.8 Hz, 1H), 2.62 (s, 3H), 2.36 (td, J=8.4, 12.0 Hz, 1H), 2.28-2.19 (m, 1H), 2.08 (br dd, J=2.8, 8.4 Hz, 1H), 1.82 (d, J=8.4 Hz, 1H), 1.45 (s, 3H), 1.23-1.12 (m, 2H), 1.11-1.00 (m, 2H).

### Example 69

### 6-[3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylic acid (Compound 531)

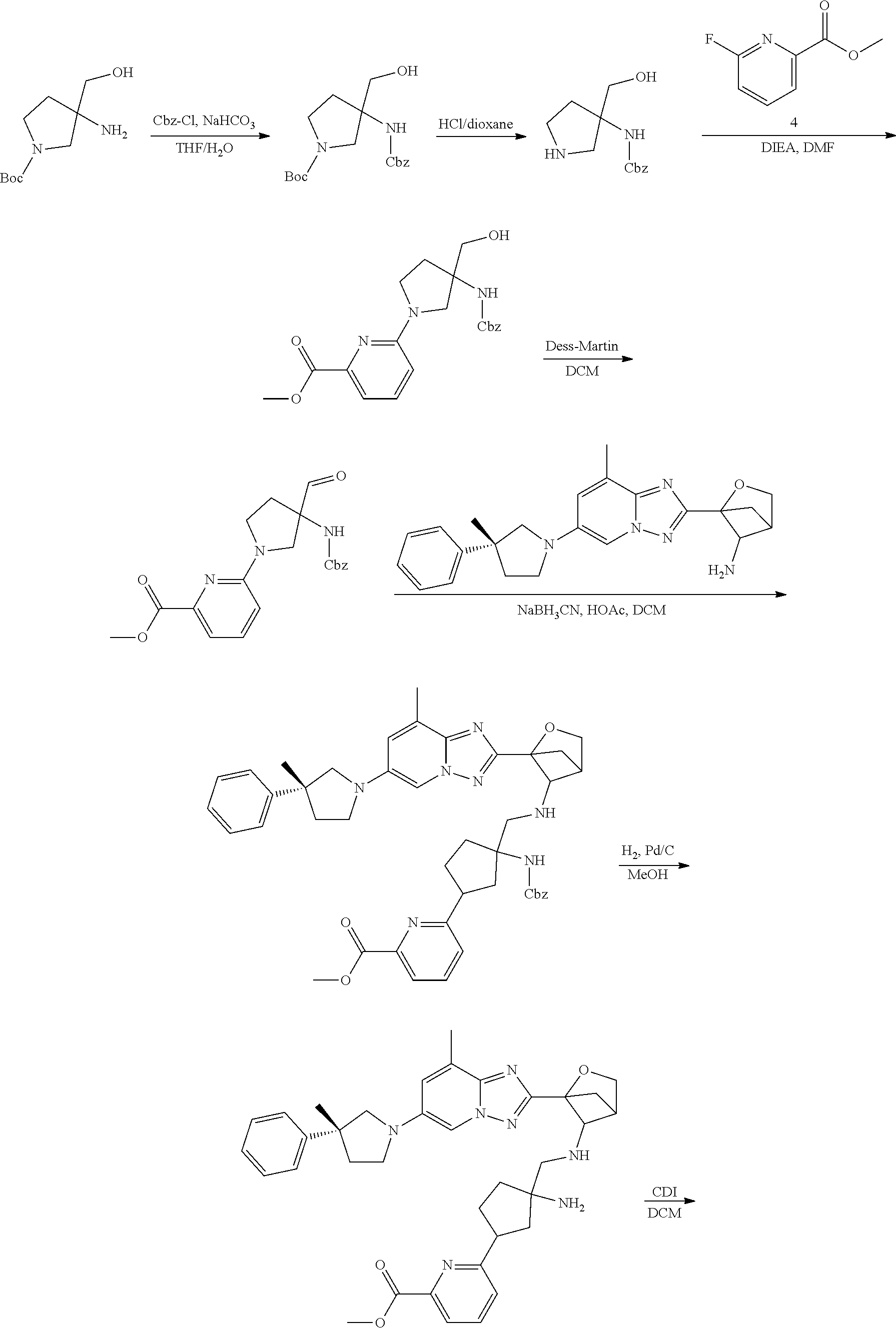

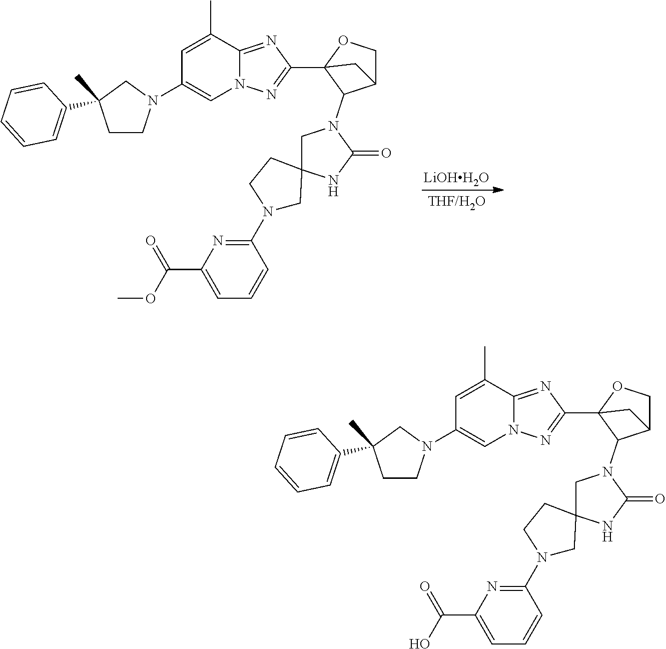

### Step 1: tert-butyl 3-(benzyloxycarbonylamino)-3-(hydroxymethyl)pyrrolidine-1-carboxylate

To a solution of tert-butyl 3-amino-3-(hydroxymethyl)pyrrolidine-1-carboxylate (1 g, 4.62 mmol, 1 eq) in THF (7 mL) and Water (2 mL) was added CbzCl (946.53 mg, 5.55 mmol, 792.07 μL, 1.2 eq) and NaHCO₃ (1.55 g, 18.49 mmol, 719.63 μL, 4 eq) The mixture was stirred at 25° C. for 16 h. LCMS showed starting material was consumed and the desired mass was detected. The mixture was concentrated in vacuum and extracted with EtOAc (10 mL×3). The combined organic layer was washed with brine (10 mL), dried over sodium sulfate, concentrated in vacuum to afford crude. The residue was purified by flash silica gel chromatography (SiO₂, PE:EtOAc=2:1, Rf=0.3) to give compound tert-butyl 3-(benzyloxycarbonylamino)-3-(hydroxymethyl) pyrrolidine-1-carboxylate (1.2 g, 74.07% yield) as a white solid.

LC-MS [ESI, M+23]: 373.1

¹H NMR (400 MHz, CHLOROFORM-d) δ=7.49-7.29 (m, 4H), 5.13 (br s, 1H), 5.08 (s, 2H), 3.79 (s, 2H), 3.54-3.39 (m, 4H), 2.05 (s, 2H), 1.45 (s, 9H).

### Step 2: benzyl N-[3-(hydroxymethyl)pyrrolidin-3-yl]carbamate

To a solution of tert-butyl 3-(benzyloxycarbonylamino)-3-(hydroxymethyl)pyrrolidine-1-carboxylate (1 g, 2.85 mmol, 1 eq) in DCM (3 mL) was added HCl/dioxane (2 M, 7.13 mL, 5 eq).The mixture was stirred at 25° C. for 1 h. LCMS showed starting material was consumed and the desired mass was detected. The reaction was filtered and the filtered concentrated under reduced pressure to give compound benzyl N-[3-(hydroxymethyl)pyrrolidin-3-yl]carbamate (1.2 g, crude, HCl) as a white solid.

LC-MS [ESI, M+1]:251.0

### Step 3: methyl 6-[3-(benzyloxycarbonylamino)-3-(hydroxymethyl)pyrrolidin-1-yl]pyridine-2-carboxylate

To a solution of benzyl N-[3-(hydroxymethyl)pyrrolidin-3-yl]carbamate (1.11 g, 3.87 mmol, 1.2 eq, HCl) and methyl 6-fluoropyridine-2-carboxylate (500 mg, 3.22 mmol, 1 eq) in DMF (5 mL) was added DIEA (1.25 g, 9.67 mmol, 1.68 mL, 3 eq). The mixture was stirred at 100° C. for 2 h. LCMS showed starting material was consumed and the desired mass was detected. The mixture was concentrated in vacuum and extracted with EtOAc (3 mL×3). The combined organic layer was washed with brine (3 mL), dried over sodium sulfate, concentrated in vacuum to afford crude. The residue was purified by flash silica gel chromatography (SiO₂, PE:EtOAc=5:1, Rf=0.3) to give compound methyl 6-[3-(benzyloxycarbonylamino)-3-(hydroxymethyl) pyrrolidin-1-yl]pyridine-2-carboxylate (700 mg, 56.35% yield) as a white solid.

LC-MS [ESI, M+1]: 386.1.

¹H NMR (400 MHz, CHLOROFORM-d) δ=7.55 (dd, J=7.6, 8.4 Hz, 1H), 7.42-7.29 (m, 6H), 6.54 (d, J=8.4 Hz, 1H), 5.25 (br s, 1H), 5.08 (s, 2H), 3.93 (s, 3H), 3.87 (s, 2H), 3.77-3.62 (m, 4H), 2.45-2.16 (m, 2H).

### Step 4: methyl 6-[3-(benzyloxycarbonylamino)-3-formyl-pyrrolidin-1-yl]pyridine-2-carboxylate

A mixture of methyl 6-[3-(benzyloxycarbonylamino)-3-(hydroxymethyl)pyrrolidin-1-yl]pyridine-2-carboxylate (600 mg, 1.56 mmol, 1 eq) and Dess-Martin (660.29 mg, 1.56 mmol, 482.32 μL, 1 eq) in DCM (6 mL) was degassed and purged with N₂ for 3 times, and then the mixture was stirred at 0-25° C. for 1 h under N₂ atmosphere. LCMS showed starting material was consumed and the desired mass was detected. The mixture was quenched with Na₂SO₃ (10 mL), and extracted with dichloromethane (10 mL×3). The combined organic layer was washed with brine (1 mL), dried over sodium sulfate, concentrated in vacuum to afford compound methyl 6-[3-(benzyloxycarbonylamino)-3-formyl-pyrrolidin-1-yl]pyridine-2-carboxylate (400 mg, 67.02% yield) as a white solid.

LC-MS [ESI, M+1]: 384.0

¹H NMR (400 MHz, CHLOROFORM-d) δ=9.57 (s, 1H), 7.49 (t, J=8.0 Hz, 1H), 7.34 (d, J=7.4 Hz, 1H), 7.31-7.26 (m, 4H), 7.25-7.16 (m, 1H), 6.48 (br d, J=8.4 Hz, 1H), 5.41 (br s, 1H), 5.11-4.98 (m, 2H), 3.93 (d, J=11.6 Hz, 1H), 3.84 (s, 3H), 3.73 (br d, J=11.6 Hz, 1H), 3.63 (br t, J=6.8 Hz, 2H), 2.41 (td, J=7.6, 13.2 Hz, 1H), 2.31-2.17 (m, 1H).

### Step 5: methyl 6-[3-(benzyloxycarbonylamino)-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate

To a solution of methyl 6-[3-(benzyloxycarbonylamino)-3-formyl-pyrrolidin-1-yl]pyridine-2-carboxylate (295.31 mg, 770.23 μmol, 1 eq) and 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (300 mg, 770.23 μmol, 1 eq) in MeOH (3 mL) was added sodium; cyanoboranuide (96.81 mg, 1.54 mmol, 2 eq) and acetic acid (23.13 mg, 385.12 μmol, 22.05 μL, 0.5 eq). The mixture was stirred at 0° C. for 2 h. LCMS showed starting material was consumed and the desired mass was detected. The mixture was concentrated in vacuum and dissolved with DCM (6 mL). The combined organic layer was washed with brine (3 mL), dried over sodium sulfate, concentrated in vacuum to afford crude. The residue was purified by flash silica gel chromatography (SiO₂, PE:EtOAc=10:1, Rf=0.3) to give compound methyl 6-[3-(benzyloxycarbonylamino)-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate (crude) as a white solid.

LC-MS [ESI, M+1]: 757.8

¹H NMR (400 MHz, CHLOROFORM-d) δ=7.56 (s, 1H), 7.51-7.40 (m, 1H), 7.37-7.29 (m, 4H), 7.26-7.17 (m, 4H), 6.86 (dd, J=0.8, 6.4 Hz, 1H), 6.56-6.38 (m, 1H), 5.99-5.63 (m, 1H), 5.26 (s, 1H), 5.13-4.89 (m, 2H), 3.94-3.83 (m, 4H), 3.83-3.76 (m, 1H), 3.74-3.65 (m, 1H), 3.63-3.56 (m, 1H), 3.54-3.37 (m, 5H), 3.31 (dt, J=2.8, 8.3 Hz, 1H), 3.19 (br d, J=2.4 Hz, 1H), 3.06-2.91 (m, 2H), 2.87-2.79 (m, 1H), 2.52 (d, J=11.2 Hz, 3H), 2.39-2.27 (m, 2H), 2.24-2.13 (m, 2H), 2.06-1.95 (m, 2H), 1.82 (br d, J=8.0 Hz, 1H), 1.70 (dd, J=1.0, 8.4 Hz, 1H), 1.41 (s, 3H).

### Step 6:methyl 6-[3-amino-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate

To a solution of methyl 6-[3-(benzyloxycarbonylamino)-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate (200 mg, 264.24 μmol, 1 eq) in MeOH (3 mL) and THF (3 mL) was added Pd/C (28.12 mg, 26.42 μmol, 10% purity, 0.1 eq) under N₂ atmosphere. The suspension was degassed and purged with H₂ for 3 times. The mixture was stirred under H₂ (15 Psi) at 25° C. for 12 h. LCMS showed starting material was consumed and the desired mass was detected. The reaction was filtered and the filtered concentrated under reduced pressure to give a residue. The crude product was purified by reversed-phase HPLC (0.1% FA condition) to give compound methyl 6-[3-amino-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate (70 mg, 42.54% yield) as a white solid.

LC-MS [ESI, M+1]: 623.3.

¹H NMR (400 MHz, CHLOROFORM-d) δ=7.58 (s, 1H), 7.50-7.38 (m, 1H), 7.35-7.29 (m, 2H), 7.27-7.23 (m, 2H), 7.23-7.17 (m, 1H), 6.91-6.81 (m, 1H), 6.49-6.32 (m, 1H), 4.81 (br s, 2H), 3.88 (s, 2H), 3.84 (d, J=3.2 Hz, 2H), 3.55-3.42 (m, 6H), 3.39-3.26 (m, 2H), 3.23 (d, J=2.8 Hz, 1H), 2.94-2.70 (m, 3H), 2.59-2.50 (m, 3H), 2.39-2.25 (m, 1H), 2.23-2.12 (m, 1H), 2.12-1.93 (m, 2H), 1.91-1.80 (m, 1H), 1.70 (d, J=8.4 Hz, 1H), 1.42-1.38 (m, 3H).

### Step 7: methyl 6-[3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylate

To a solution of methyl 6-[3-amino-3-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]pyrrolidin-1-yl]pyridine-2-carboxylate (60 mg, 96.35 μmol, 1 eq) in ACN (1 mL) was added CDI (78.11 mg, 481.73 μmol, 5 eq). The mixture was stirred at 80° C. for 1 h. LCMS showed starting material was consumed and the desired mass was detected. The reaction was filtered and the filtered concentrated under reduced pressure to give a residue. The residue was purified by flash silica gel chromatography (SiO₂, PE:EtOAc=0:1, Rf=0.3) to give compound methyl 6-[3-[1-[8-methyl-6-[(3R)-3-methyl-3-phenyl-pyrrolidin-1-yl]-triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylate (50 mg, 79.99% yield) as a white solid.

LC-MS [ESI, M+1]: 649.6.

### Step 8:6-[3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylic acid

To a solution of methyl 6-[3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylate (40 mg, 61.66 μmol, 1 eq) in THF (1 mL) and Water (0.5 mL) was added LiOH·H₂O (7.76 mg, 184.97 μmol, 3 eq).The mixture was stirred at 25° C. for 0.5 h. LCMS showed starting material was consumed and the desired mass was detected. The reaction was filtered and the filtered concentrated under reduced pressure to give a residue. The residue was purified by prep-HPLC (column: CD02-Waters Xbidge BEH C18 150*25*10 um; mobile phase: [water(NH₄HCO₃)—ACN]; gradient:20%-50% B over 10 min). The result solution was lyophilized to give compound 6-[3-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-oxo-1,3,7-triazaspiro[4.4]nonan-7-yl]pyridine-2-carboxylic acid (1.9 mg, 9.19% yield, 96.755% purity) as a white solid.

LC-MS [ESI, M+1]: 635.2.

¹H NMR (400 MHz, CHLOROFORM-d) δ=7.73-7.63 (m, 1H), 7.62 (d, J=1.6 Hz, 1H), 7.51 (d, J=7.2 Hz, 1H), 7.41-7.34 (m, 2H), 7.33-7.28 (m, 2H), 7.26-7.21 (m, 1H), 6.95 (s, 1H), 6.59 (d, J=8.4 Hz, 1H), 4.74 (br s, 1H), 4.11-3.93 (m, 3H), 3.77 (d, J=9.6 Hz, 1H), 3.66-3.34 (m, 10H), 2.60 (s, 3H), 2.38 (td, J=8.0, 12.2 Hz, 1H), 2.28-2.23 (m, 1H), 2.19-2.14 (m, 1H), 2.13-2.08 (m, 2H), 1.96 (br d, J=8.4 Hz, 1H), 1.46 (s, 3H).

| Compound | | |
|---|---|---|
| Number | Synthesis | LC/MS |
| 531 | Example 69 | m/z 635.2 [M + H]⁺ |
| 515 | Similar to Example 69 | m/z 500.2 [M + H]⁺ |
| 516 | Similar to Example 69 | m/z 621.3 [M + H]⁺ |
| 517 | Similar to Example 69 | m/z 642.5 [M + H]⁺ |
| 518 | Similar to Example 69 | m/z 644.4 [M + H]⁺ |
| 519 | Similar to Example 69 | m/z 621.4 [M + H]⁺ |
| 520 | Similar to Example 69 | m/z 621.4 [M + H]⁺ |

### Example 70

### 7-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-5,7-diazaspiro[3.4]octan-6-one (Compound 532)

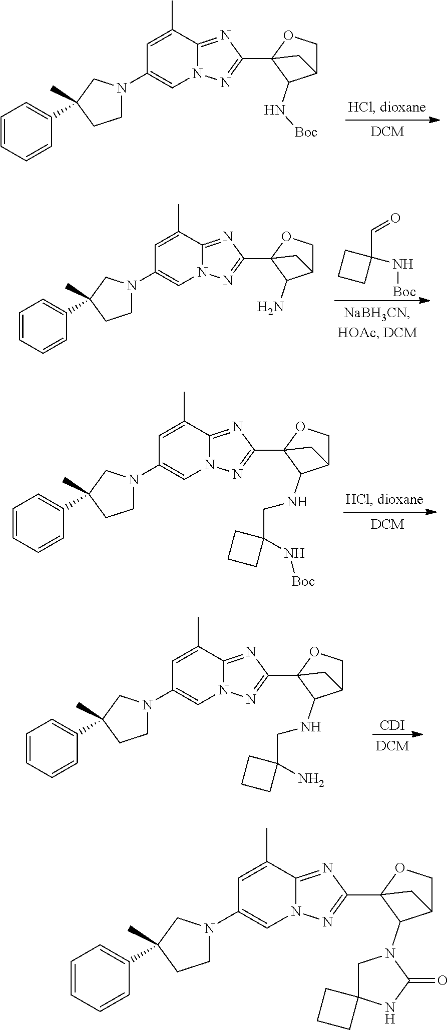

### Step 1: 1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine

To a solution of tert-butyl N-[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (1 g, 2.04 mmol, 1 eq) in DCM (2 mL) was added HCl/dioxane (2 M, 1.02 mL, 1 eq). The mixture was stirred at 25° C. for 1 h. LCMS showed starting material was consumed and one main peak with desired mass was detected. The reaction mixture was concentrated under the reduced pressure to give compound 1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine (850 mg, crude, HCl) as a yellow solid.

LC-MS [ESI, M+1]: 390.5

### Step 2: tert-butyl (1-(((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)amino)methyl)cyclobutyl)carbamate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (100 mg, 256.74 μmol, 1 eq) and tert-butyl N-(1-formylcyclobutyl)carbamate (102.31 mg, 513.49 μmol, 2 eq) in DCM (5 mL) was added NaBH₃CN (48.40 mg, 770.23 μmol, 3 eq) and HOAc (15.42 mg, 256.74 μmol, 14.70 μL, 1 eq). The mixture was stirred at 25° C. for 1 h. LCMS showed starting material was consumed and one main peak with desired mass was detected. The reaction mixture was concentrated under the reduced pressure to give the product. The residue was purified by prep-TLC (SiO₂, PE:EtOAc=0:1, Rf=0.3) and eluted with 60 mL (10% methanol in DCM) to give compound tert-butyl (1-(((1-(8-methyl-6-((R)-3-methyl-3-phenylpyrrolidin-1-yl)-triazolo[1,5-a]pyridin-2-yl)-2-oxabicyclo[2.1.1]hexan-5-yl)amino)methyl)cyclobutyl)carbamate (80 mg, 139.68 μmol, 54.40% yield) as a yellow gum.

LC-MS [ESI, M+1]: 573.5

### Step 3: N-((1-aminocyclobutyl)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine

To a solution of tert-butyl N-[1-[[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]cyclobutyl]carbamate (80 mg, 139.68 μmol, 1 eq) in DCM (3 mL) was added HCl/dioxane (2 M, 26.19 L). The mixture was stirred at 25° C. for 10 min. LCMS showed starting material was consumed and one main peak with desired mass was detected. The mixture was concentrated to give residue, the residue was adjusted PH around 6 by NaHCO₃, solid was precipitate out, and the mixture was filtered and the solid was concentrated under reduced pressure to give a residue. The residue was purified by prep-HPLC (column: Phenomenex C18 150*25 mm*10 um; mobile phase: [water(NH₄HCO₃)—ACN]; gradient:0%-30% B over 10 min) and lyophilized to give compound N-((1-aminocyclobutyl)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-amine (50.57 mg, 106.15 μmol, 76.00% yield, 99.209% purity) as a white solid.

LC-MS [ESI, M+1]: 473.3

¹H NMR (CHLOROFORM-d, 400 MHz): δ=7.64 (s, 1H), 7.35 (d, J=7.2 Hz, 2H), 7.27-7.33 (m, 2H), 7.21-7.27 (m, 1H), 6.93 (s, 1H), 3.88-3.99 (m, 2H), 3.58 (d, J=8.8 Hz, 1H), 3.47 (t, J=8.8 Hz, 2H), 3.38 (d, J=4.0 Hz, 1H), 3.28 (d, J=2.8 Hz, 1H), 2.92 (t, J=2.8 Hz, 1H), 2.75 (d, J=12.0 Hz, 1H), 2.56-2.65 (m, 4H), 2.35 (s, 1H), 2.26 (dd, J=7.2, 3.6 Hz, 1H), 1.93-1.98 (m, 3H), 1.66-1.81 (m, 4H), 1.55 (d, J=8.0 Hz, 1H), 1.46 ppm (s, 3H)

### Step 4: 7-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-5,7-diazaspiro[3.4]octan-6-one

To a solution of N-[(1-aminocyclobutyl)methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (15 mg, 31.74 μmol, 1 eq) in DCM (3 mL) was added CDI (25.73 mg, 158.69 μmol, 5 eq). The mixture was stirred at 40° C. for 1 h. LCMS showed starting material was consumed and one main peak with desired mass was detected. The reaction mixture was concentrated under the reduced pressure to give compound 7-(1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-5,7-diazaspiro[3.4]octan-6-one (12.29 mg, 24.55 μmol, 77.37% yield, 99.619% purity) as a white solid.

LC-MS [ESI, M+1]: 499.3

¹H NMR (CHLOROFORM-d, 400 MHz): δ=7.66 (d, J=1.6 Hz, 1H), 7.36 (d, J=7.2 Hz, 2H), 7.33 (d, J=1.2 Hz, 2H), 7.26 (s, 1H), 6.96 (s, 1H), 4.59 (s, 1H), 4.00 (s, 2H), 3.97 (d, J=3.2 Hz, 1H), 3.81 (d, J=9.2 Hz, 1H), 3.60 (d, J=8.8 Hz, 1H), 3.46 (dd, J=11.2, 9.2 Hz, 3H), 3.40 (td, J=8.8, 3.7 Hz, 1H), 3.35 (t, J=3.2 Hz, 1H), 2.64 (s, 3H), 2.36 (s, 1H), 2.19-2.29 (m, 2H), 2.12-2.19 (m, 1H), 2.07 (d, J=5.2 Hz, 2H), 1.99 (qd, J=8.0, 3.6 Hz, 1H), 1.92 (d, J=8.4 Hz, 1H), 1.52-1.64 (m, 2H), 1.47 ppm (s, 3H)

### Example 71

### 6-(3-methyl-3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamoyl)azetidin-1-yl)picolinic acid (Compound 533)

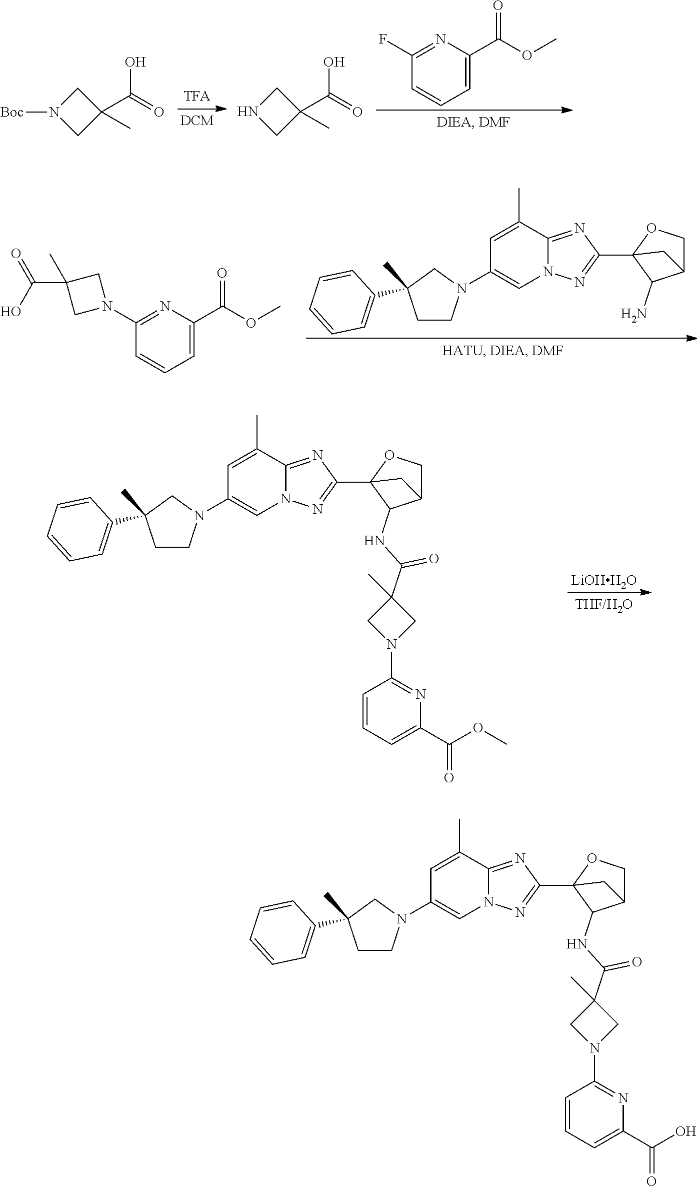

### Step 1: 3-methylazetidine-3-carboxylic acid

To a solution of 1-tert-butoxycarbonyl-3-methyl-azetidine-3-carboxylic acid (50 mg, 232.29 μmol, 1 eq) in DCM (0.5 mL) was added TFA (383.75 mg, 3.37 mmol, 0.25 mL, 14.49 eq). The mixture was stirred at 25° C. for 0.5 h. LCMS showed starting material was consumed and the desired mass was detected. The reaction mixture was concentrated under the reduced pressure to give compound 3-methylazetidine-3-carboxylic acid (50 mg, crude, TFA) as colorless oil.

LC-MS [ESI, M+1]:116.2.

### Step 2: 1-(6-(methoxycarbonyl)pyridin-2-yl)-3-methylazetidine-3-carboxylic acid

To a solution of 3-methylazetidine-3-carboxylic acid (500 mg, 2.18 mmol, 1 eq, TFA) in DMF (3 mL) was added DIEA (846.00 mg, 6.55 mmol, 1.14 mL, 3 eq) and methyl 6-fluoropyridine-2-carboxylate (338.48 mg, 2.18 mmol, 1 eq). The mixture was stirred at 100° C. for 1 h. LCMS showed starting material was consumed and the desired mass was detected. The mixture was quenched with water (30 mL) and extracted with ethyl acetate (50 mL×2). The combined organic phase was washed with brine (80 mL), dried with anhydrous Na₂SO₄, filtered and concentrated in vacuum. The residue was purified by column chromatography (SiO₂, PE:EtOAc=0:1, Rf=0.61) to give compound 1-(6-methoxycarbonyl-2-pyridyl)-3-methyl-azetidine-3-carboxylic acid (130 mg, 493.51 μmol, 22.62% yield, 95% purity) as brown oil.

LC-MS [ESI, M+1]:251.0.

¹H NMR (400 MHz, CHLOROFORM-d) δ=7.63-7.52 (m, 1H), 7.44 (d, J=7.2 Hz, 1H), 6.48 (d, J=8.4 Hz, 1H), 4.43 (d, J=8.0 Hz, 2H), 3.94 (s, 3H), 3.90 (d, J=8.0 Hz, 2H), 1.66 (s, 3H)

### Step 3: methyl 6-(3-methyl-3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamoyl)azetidin-1-yl)picolinate

To a solution of 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (95 mg, 243.91 μmol, 1 eq) in DMF (1 mL) was added 1-(6-methoxycarbonyl-2-pyridyl)-3-methyl-azetidine-3-carboxylic acid (122.08 mg, 487.81 μmol, 2 eq), HATU (111.29 mg, 292.69 μmol, 1.2 eq) and DIEA (94.57 mg, 731.72 μmol, 127.45 μL, 3 eq). The mixture was stirred at 25° C. for 1 h. LCMS showed starting material was consumed and the desired mass was detected. The reaction mixture was concentrated under the reduced pressure to give the crude product. The residue was purified by prep-HPLC (FA condition column: CD04-Welch Utimate C18 150*25*7 um; mobile phase: [water(FA)—ACN]; gradient:33%-63% B over 10 min) to give compound methyl 6-[3-methyl-3-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamoyl]azetidin-1-yl]pyridine-2-carboxylate (80 mg, 115.81 μmol, 47.48% yield, 90% purity) as yellow solid.

LC-MS [ESI, M+1]:622.3.

¹H NMR (400 MHz, CHLOROFORM-d) δ=7.62 (d, J=1.6 Hz, 1H), 7.58-7.51 (m, 1H), 7.43 (d, J=7.2 Hz, 1H), 7.39-7.34 (m, 2H), 7.33-7.29 (m, 2H), 7.27-7.22 (m, 1H), 6.95 (s, 1H), 6.56-6.39 (m, 2H), 4.36 (dd, J=8.0, 11.2 Hz, 2H), 4.31 (dd, J=2.8, 6.8 Hz, 1H), 4.01 (d, J=6.4 Hz, 1H), 3.92 (s, 3H), 3.90-3.80 (m, 3H), 3.59 (d, J=8.8 Hz, 1H), 3.55-3.44 (m, 2H), 3.38 (dt, J=3.6, 8.8 Hz, 1H), 3.19 (t, J=2.8 Hz, 1H), 2.60 (s, 3H), 2.37 (td, J=8.4, 12.0 Hz, 1H), 2.30-2.20 (m, 1H), 2.18 (dd, J=3.2, 8.4 Hz, 1H), 1.94 (d, J=8.8 Hz, 1H), 1.63 (s, 3H), 1.46 (s, 3H)

### Step 4: 6-(3-methyl-3-((1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)carbamoyl)azetidin-1-yl)picolinic acid

To a solution of methyl 6-[3-methyl-3-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamoyl]azetidin-1-yl]pyridine-2-carboxylate (20 mg, 32.17 μmol, 1 eq) in THF (0.6 mL) and H₂O (0.2 mL) was added LiOH·H₂O (2.70 mg, 64.34 μmol, 2 eq). The mixture was stirred at 25° C. for 1 h. LCMS showed starting material was consumed and the desired mass was detected. The reaction mixture was concentrated under the reduced pressure to give the crude product. The residue was purified by prep-HPLC (FA condition column: CD01-Phenomenex luna C18 150*25*10 um; mobile phase: [water(FA)—ACN]; gradient:22%-52% B over 10 min) to give compound 6-[3-methyl-3-[[1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamoyl]azetidin-1-yl]pyridine-2-carboxylic acid (11.55 mg, 18.90 μmol, 58.74% yield, 99.424% purity) as white solid.

LC-MS [ESI, M+1]:608.3.

¹H NMR (400 MHz, CHLOROFORM-d) δ=7.72-7.57 (m, 2H), 7.51 (d, J=7.2 Hz, 1H), 7.40-7.33 (m, 2H), 7.33-7.29 (m, 2H), 7.26 (br s, 1H), 6.95 (s, 1H), 6.58-6.43 (m, 2H), 4.37 (dd, J=8.0, 14.4 Hz, 2H), 4.30 (dd, J=2.4, 6.8 Hz, 1H), 4.03 (d, J=6.4 Hz, 1H), 3.89-3.78 (m, 3H), 3.58 (br d, J=8.8 Hz, 1H), 3.54-3.43 (m, 2H), 3.37 (dt, J=3.2, 8.4 Hz, 1H), 3.21 (br s, 1H), 2.60 (s, 3H), 2.40-2.33 (m, 1H), 2.26 (dt, J=3.6, 7.8 Hz, 1H), 2.19 (br dd, J=2.8, 8.4 Hz, 1H), 1.96 (d, J=8.6 Hz, 1H), 1.67 (s, 3H), 1.46 (s, 3H).

### Example 72

### 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxamide

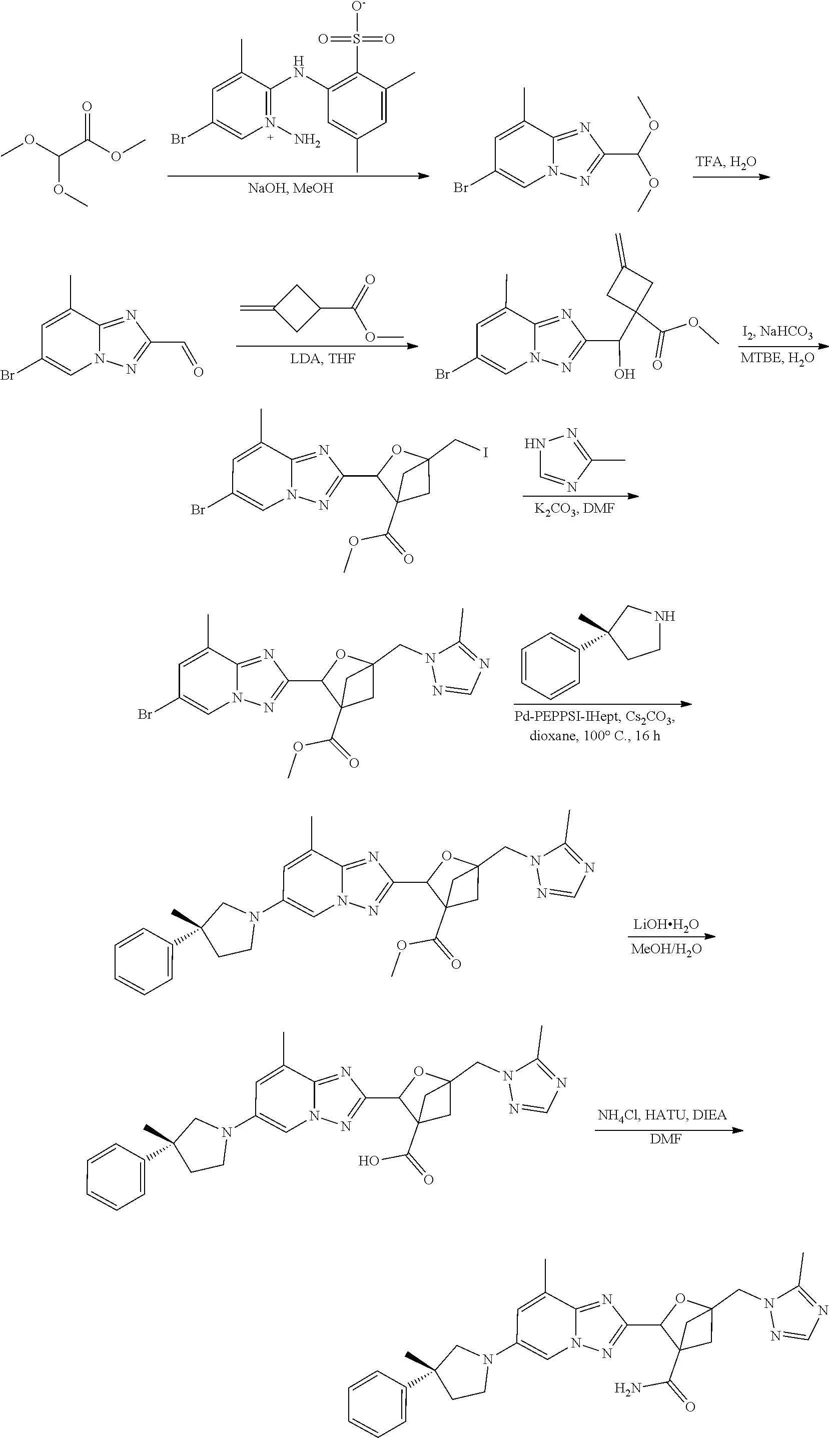

### Step 1: 6-bromo-2-(dimethoxymethyl)-8-methyl-[1,2,4]triazolo[1,5-a]pyridine

To a solution of 5-bromo-3-methyl-pyridin-1-ium-1,2-diamine;2,4,6-trimethylbenzenesulfonate (10 g, 24.86 mmol, 1 eq) and methyl 2,2-dimethoxyacetate (6.67 g, 49.71 mmol, 6.10 mL, 2 eq) in MeOH (200 mL) was added NaOH (1.49 g, 37.29 mmol, 1.5 eq). The mixture was stirred at 70° C. for 12 hr. LCMS showed starting material consumed competely. Several new peaks were shown on LCMS and 55% of desired mass was detected. The reaction mixture was filtered and concentrated under reduced pressure to give a residue. The residue was purified by flash silica gel chromatography (ISCO®; 40 g SepaFlash® Silica Flash Column, Eluent of 0~42% Ethyl acetate/Petroleum ethergradient @70 mL/min, PE:EtOAc=1:1, Rf=0.4) to give compound 6-bromo-2-(dimethoxymethyl)-8-methyl-[1,2,4]triazolo[1,5-a]pyridine (5.36 g, 75% yield) as a yellow solid.

LC-MS [ESI, M-32]: 253.6.

¹H NMR (400 MHz, CHLOROFORM-d) δ=8.57 (d, J=0.4 Hz, 1H), 7.40 (d, J=0.8 Hz, 1H), 5.73 (d, J=2.0 Hz, 1H), 3.49 (d, J=2.0 Hz, 6H), 2.65 (s, 3H)

### Step 2: 6-bromo-8-methyl-[1,2,4]triazolo[1,5-a]pyridine-2-carbaldehyde

To a solution of 6-bromo-2-(dimethoxymethyl)-8-methyl-[1,2,4]triazolo[1,5-a]pyridine (6.2 g, 21.67 mmol, 1 eq) in H₂O (5 mL) was added dropwise TFA (20 mL) dropwise at 0° C. After addition, the mixture was stirred at 80° C. for 1 hr. LCMS showed starting material consumed competely. Several new peaks were shown on LCMS and 56% of desired mass was detected. The reaction mixture was concentrated under reduced pressure to give a residue. The residue was diluted with NaHCO₃50 mL and extracted with ethyl acetate 240 mL (80 mL×3). The combined organic layers were dried over anhydrous sodium sulfate, filtered and concentrated under reduced pressure to give a residue 6-bromo-8-methyl-[1,2,4]triazolo[1,5-a]pyridine-2-carbaldehyde (4.73 g, 62% yield) as a yellow solid.

LC-MS [ESI, M+1]: 239.9.

¹H NMR (400 MHz, CHLOROFORM-d) δ=10.22 (s, 1H), 8.67 (d, J=0.8 Hz, 1H), 7.51 (s, 1H), 2.71 (s, 3H)

### Step 3: methyl 1-[(6-bromo-8-methyl-[1,2,4]triazolo[1,5-a]pyridin-2-yl)-hydroxy-methyl]-3-methylenecyclobutanecarboxylate

To a solution of methyl 3-methylenecyclobutanecarboxylate (1.68 g, 13.33 mmol, 1 eq) in THF (200 mL) was added LDA (2 M, 16.66 mL, 2.5 eq) at −78° C. under N₂ atmosphere, after 1 hr, 6-bromo-8-methyl-[1,2,4]triazolo[1,5-a]pyridine-2-carbaldehyde (3.2 g, 13.33 mmol, 1 eq) was added in reaction mixture, the resulting mixture was stirred for another 1 hr at −78° C. LCMS showed 3% of starting material remained. Several new peaks were shown on LCMS and 29% of desired mass was detected. The reaction mixture was quenched by addition NH₄Cl 25 mL at −20° C., and then diluted with H₂O 100 mL and extracted with ethyl acetate 300 mL (100 mL×3). The combined organic layers were washed with brine 240 mL (80 mL×3), dried over anhydrous sodium sulfate, filtered and concentrated under reduced pressure to give a residue. The residue was purified by flash silica gel chromatography (ISCO®; 40 g SepaFlash® Silica Flash Column, Eluent of 0~50% Ethyl acetate/Petroleum ethergradient @60 mL/min, PE:EtOAc=1:1, Rf=0.5) to give compound methyl 1-[(6-bromo-8-methyl-[1,2,4]triazolo[1,5-a]pyridin-2-yl)-hydroxy-methyl]-3-methylenecyclobutanecarboxylate (970 mg, 17% yield) as a yellow solid.

LC-MS [ESI, M-32]: 366.8.

¹H NMR (400 MHz, CHLOROFORM-d) δ=8.52 (d, J=0.8 Hz, 1H), 7.37 (s, 1H), 5.19 (d, J=9.2 Hz, 1H), 4.84 (t, J=2.0 Hz, 2H), 3.89 (d, J=9.2 Hz, 1H), 3.77 (s, 3H), 3.26-3.12 (m, 2H), 3.10-2.95 (m, 2H), 2.60 (s, 3H)

### Step 4: methyl 3-(6-bromo-8-methyl-[1,2,4]triazolo[1,5-a]pyridin-2-yl)-1-[(5-methyl-1,2,4-triazol-1-yl)methyl]-2-oxabicyclo[2.1.1]hexane-4-carboxylate

To a solution of methyl 3-(6-bromo-8-methyl-[1,2,4]triazolo[1,5-a]pyridin-2-yl)-1-(iodomethyl)-2-oxabicyclo[2.1.1]hexane-4-carboxylate (300 mg, 609.63 μmol, 1 eq) in DMF (3 mL) was added 3-methyl-1H-1,2,4-triazole (101.31 mg, 1.22 mmol, 2 eq) and K₂CO₃ (210.63 mg, 1.52 mmol, 2.5 eq). The mixture was stirred at 80° C. for 1 h. LCMS showed starting material consumed. Several new peaks were shown on LCMS and 79.26% of desired compound was detected. The reaction mixture was filtered and concentrated, and then was diluted with H₂O (20 mL) and extracted with EtOAc (30 mL×3). The combined organic layers were washed with brine (20 mL×5), dried over Na₂SO₄, filtered and concentrated under reduced pressure to give a residue. The residue was purified by prep-HPLC (TFA condition) to give compound methyl 3-(6-bromo-8-methyl-[1,2,4]triazolo[1,5-a]pyridin-2-yl)-1-[(5-methyl-1,2,4-triazol-1-yl)methyl]-2-oxabicyclo[2.1.1]hexane-4-carboxylate (84 mg, 30.09% yield) as white solid.

LC-MS [ESI, M+1]: 447.1.

¹H NMR (400 MHz, CHLOROFORM-d) δ=8.59 (s, 1H), 8.19 (s, 1H), 7.48 (s, 1H), 5.46 (s, 1H), 4.71-4.50 (m, 2H), 3.70 (s, 3H), 2.74 (s, 3H), 2.61 (s, 3H), 2.57-2.48 (m, 1H), 2.43 (d, J=7.6 Hz, 1H), 2.28 (d, J=8.0 Hz, 1H), 2.21 (br dd, J=8.0, 10.0 Hz, 1H)

### Step 5: methyl 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylate

A mixture of methyl 3-(6-bromo-8-methyl-[1,2,4]triazolo[1,5-a]pyridin-2-yl)-1-[(5-methyl-1,2,4-triazol-1-yl)methyl]-2-oxabicyclo[2.1.1]hexane-4-carboxylate (84.00 mg, 187.80 μmol, 1 eq), (3R)-3-methyl-3-phenyl-pyrrolidine (30.28 mg, 187.80 μmol, 1 eq), Cs₂CO₃ (152.97 mg, 469.50 μmol, 2.5 eq) and 1,3-bis[2,6-bis(1-propylbutyl)phenyl]-4,5-dichloro-2H-imidazol-1-ium-2-ide;3-chloropyridine;dichloropalladium (9.13 mg, 9.39 μmol, 0.05 eq) in dioxane (1 mL) was degassed and purged with N₂ for 3 times, and then the mixture was stirred at 100° C. for 16 h under N₂ atmosphere. LCMS showed starting material consumed. Several new peaks were shown on LCMS and 71.9% of desired compound was detected. The reaction mixture was filtered and concentrated to give a residue. The residue was purified by prep-HPLC (FA condition) to give compound methyl 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylate (39.18 mg, 39.54% yield) as yellow solid.

LC-MS [ESI, M+1]: 528.3

¹H NMR (400 MHz, CHLOROFORM-d) δ=7.76 (s, 1H), 7.59 (br s, 1H), 7.31 (br d, J=7.2 Hz, 2H), 7.27 (s, 1H), 7.26-7.19 (m, 2H), 6.88 (br s, 1H), 5.41-5.34 (m, 1H), 4.58-4.38 (m, 2H), 3.67-3.60 (m, 3H), 3.54 (br d, J=8.8 Hz, 1H), 3.50-3.39 (m, 2H), 3.33 (dt, J=3.6, 8.4 Hz, 1H), 2.68 (br dd, J=8.0, 10.2 Hz, 1H), 2.54 (s, 3H), 2.47 (s, 3H), 2.32 (td, J=8.4, 12.0 Hz, 1H), 2.26-2.17 (m, 2H), 2.15-2.07 (m, 2H), 1.41 (s, 3H).

### Step 6: 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylic acid

To a solution of methyl 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylate (33 mg, 62.55 μmol, 1 eq) in MeOH (0.9 mL) and H₂O (0.3 mL) was added LiOH·H₂O (5.25 mg, 125.09 μmol, 2 eq). The mixture was stirred at 25° C. for 2 h. LCMS showed starting material was consumed and one main peak with desired mass was detected. The reaction mixture was filtered and concentrated under reduced pressure to give a residue. The residue was purified by prep-HPLC (FA condition) to give compound 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylic acid (27.99 mg, 87.13% yield) as yellow solid.

LC-MS [ESI, M+1]: 514.3.

¹H NMR (400 MHz, CHLOROFORM-d) δ=7.73 (s, 1H), 7.63 (s, 1H), 7.36-7.29 (m, 2H), 7.27 (s, 1H), 7.26-7.19 (m, 2H), 6.96 (s, 1H), 5.32 (s, 1H), 4.47 (s, 2H), 3.56 (d, J=8.4 Hz, 1H), 3.51-3.40 (m, 2H), 3.35 (dt, J=3.2, 8.6 Hz, 1H), 2.50 (d, J=12.4 Hz, 6H), 2.38 (d, J=6.4 Hz, 1H), 2.36-2.30 (m, 1H), 2.27-2.18 (m, 1H), 2.16-2.06 (m, 3H), 1.42 (s, 3H)

### Step 7: 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxamide

To a solution of 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxylic acid (20.00 mg, 38.94 μmol, 1 eq) in DMF (0.5 mL) was added NH₄Cl (3.12 mg, 58.41 μmol, 1.5 eq), HATU (22.21 mg, 58.41 μmol, 1.5 eq) and DIEA (7.55 mg, 58.41 μmol, 10.17 μL, 1.5 eq). The resulting mixture was stirred at 25° C. for 2 h. LCMS showed starting material was consumed. Several new peaks were shown on LCMS and 76.81% of desired compound was detected. The reaction mixture was filtered and then was diluted with H₂O (10 mL) and extracted with EtOAc (10 mL×3). The combined organic layers were washed with brine (10 mL×5), dried over Na₂SO₄, filtered and concentrated under reduced pressure to give a residue. The residue was purified by prep-HPLC (FA condition) to give compound 3-[8-methyl-6-[(3R)-3-methyl-3-phenyl-pyrrolidin-1-yl]-[1,2,4]triazolo[1,5-a]pyridin-2-yl]-1-[(5-methyl-1,2,4-triazol-1-yl)methyl]-2-oxabicyclo[2.1.1]hexane-4-carboxamide (5.08 mg, 24.97% yield) as white solid.

LC-MS [ESI, M+1]: 513.3.

¹H NMR (400 MHz, METHANOL-d4) δ=7.86 (s, 1H), 7.72 (s, 1H), 7.39-7.29 (m, 4H), 7.24-7.16 (m, 2H), 5.28 (s, 1H), 4.68-4.55 (m, 2H), 3.60-3.48 (m, 3H), 3.42-3.34 (m, 1H), 2.68 (dd, J=7.6, 10.2 Hz, 1H), 2.55 (d, J=12.0 Hz, 6H), 2.40-2.24 (m, 3H), 2.22-2.08 (m, 2H), 1.43 (s, 3H).

| Compound | | |
|---|---|---|
| Number | Synthesis | LC/MS |
| 534 | Example 72 | m/z 513.6 [M + H]⁺ |
| 535 | Example 72 | m/z 512.62 [M + H]⁺ |
| 536 | Similar to Example 72 | m/z 464.1 [M + H]⁺ |

### Example 73

### 1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4,5-dicarboxylic acid (Compound 537)

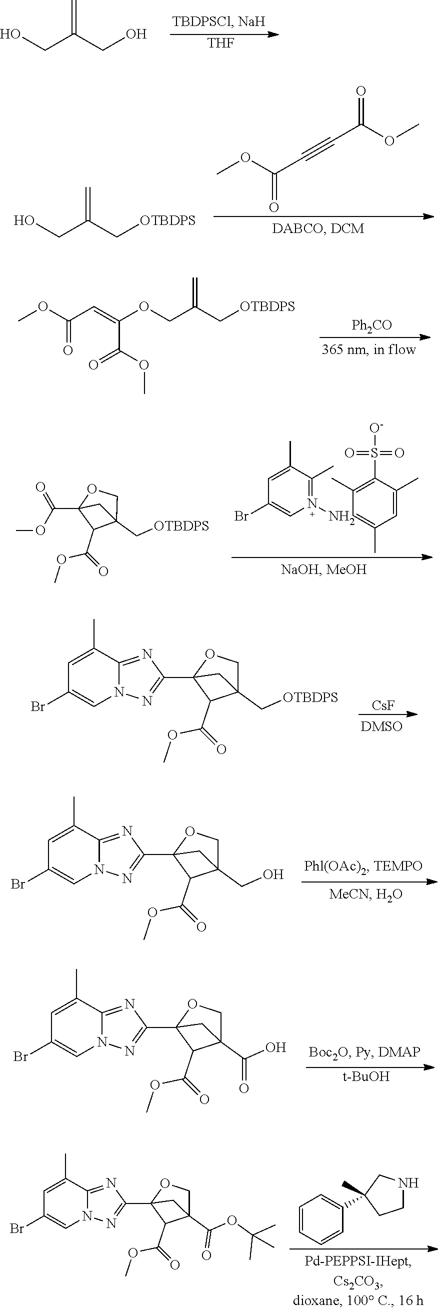

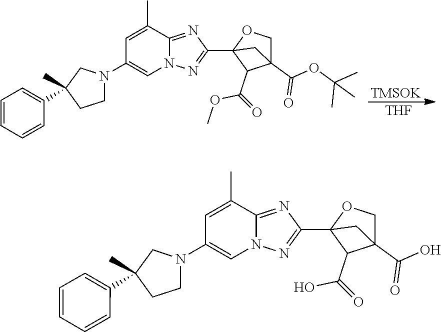 [SMILES: "TMSOK" ; COC(=O)C1C2(C(=O)OC(C)(C)C)COC1(c1nc3c(C)cc(N4CC[C@](C)(c5ccccc5)C4)cn3n1)C2 ; Cc1cc(N2CC[C@](C)(c3ccccc3)C2)cn2nc(C34CC(C(=O)O)(CO3)C4C(=O)O)nc12 ; "THF"]

### Step 1: 2-(((tert-butyldiphenylsilyl)oxy)methyl)prop-2-en-1-ol

To a solution of 2-methylenepropane-1,3-diol (20 g, 227.00 mmol, 1 eq) in THF (200 mL) was added NaH (9.08 g, 227.00 mmol, 60% purity, 1 eq) at 0° C., the mixture was stirred at 0° C. for 1 h. Then TBDPSCl (68.63 g, 249.70 mmol, 63.90 mL, 1.1 eq) was added to the mixture at same temperature, the mixture was stirred at 25° C. for 4 h. TLC indicated reactant was consumed completely and one new spot was formed. The reaction mixture was quenched by H₂O (100 mL) at 0° C., then extracted with ethyl acetate (200 mL×3), the combined organic layers dried over anhydrous sodium sulfate, filtered and the filtrate was concentrated under reduced pressure to give a residue. The residue was purified by flash silica gel chromatography (SiO₂, PE:EtOAc=5:1, Rf=0.32) to give compound 2-[[tert-butyl(diphenyl)silyl]oxymethyl]prop-2-en-1-ol (58 g, 78.2% yield) as yellow oil.

¹H NMR (400 MHz, CHLOROFORM-d) δ=7.69 (d, J=6.8 Hz, 4H), 7.49-7.34 (m, 6H), 5.14 (br d, J=14.8 Hz, 2H), 4.27 (s, 2H), 4.19 (s, 2H), 1.08 (s, 9H).

### Step 2: dimethyl 2-((2-(((tert-butyldiphenylsilyl)oxy)methyl)allyl)oxy)maleate

To a solution of dimethyl but-2-ynedioate (32.07 g, 225.68 mmol, 1 eq) and 2-[[tertbutyl(diphenyl)silyl]oxymethyl]prop-2-en-1-ol (70 g, 214.39 mmol, 0.95 eq) in DCM (700 mL) was added DABCO (5.06 g, 45.14 mmol, 0.2 eq). The mixture was stirred at 25° C. for 12 h. TLC indicated reactant was consumed and one new spot formed. The reaction was concentrated under reduced pressure to give a residue. The residue was purified by flash silica gel chromatography (SiO₂, PE:EtOAc=10:1, Rf=0.31) to give compound dimethyl (E)-2-[2-[[tert-butyl(diphenyl)silyl]oxymethyl]allyloxy]but-2-enedioate (35 g, 33.2% yield) as a colorless oil.

¹H NMR (400 MHz, CHLOROFORM-d) δ=7.67 (d, J=6.8 Hz, 4H), 7.47-7.36 (m, 6H), 6.16 (s, 1H), 5.38 (s, 1H), 5.27 (s, 1H), 4.70 (s, 2H), 4.30 (s, 2H), 3.77 (s, 3H), 3.64 (s, 3H), 1.06 (s, 9H).

### Step 3: dimethyl 4-(((tert-butyldiphenylsilyl)oxy)methyl)-2-oxabicyclo[2.1.1]hexane-1,5-dicarboxylate

To a solution of dimethyl (E)-2-[2-[[tert-butyl(diphenyl)silyl]oxymethyl]allyloxy]but-2-enedioate (45 g, 96.03 mmol, 1 eq) in ACN (800 mL) was added diphenylmethanone (1.75 g, 9.60 mmol, 0.1 eq). The mixture was stirred at 25° C. for 40 min under 365 nm. TLC indicated reactant was consumed completely and some new spots formed. The reaction was filtered and the filtered concentrated under reduced pressure to give a residue. The residue was purified by flash silica gel chromatography (SiO₂, PE:EtOAc=3:1) to give compound dimethyl 4-[[tert-butyl(diphenyl)silyl]oxymethyl]-2-oxabicyclo[2.1.1]hexane-1,5-dicarboxylate (7 g, 15.4% yield) as a colorless oil.

¹H NMR (400 MHz, CHLOROFORM-d) δ=7.69-7.61 (m, 4H), 7.48-7.38 (m, 6H), 4.07 (d, J=11.2 Hz, 1H), 3.93 (d, J=11.2 Hz, 1H), 3.90 (br d, J=6.0 Hz, 1H), 3.85 (s, 3H), 3.78 (d, J=6.0 Hz, 1H), 3.67 (s, 3H), 3.14 (s, 1H), 2.14 (d, J=7.6 Hz, 1H), 1.81 (d, J=7.6 Hz, 1H), 1.08 (s, 9H).

### Step 4: methyl 1-(6-bromo-8-methyl-[1,2,4]triazolo[1,5-a]pyridin-2-yl)-4-(((tert-butyldiphenylsilyl)oxy)methyl)-2-oxabicyclo[2.1.1]hexane-5-carboxylate

To a solution of 5-bromo-3-methyl-pyridin-1-ium-1,2-diamine;2,4,6-trimethylbenzenesulfonate (6.70 g, 16.64 mmol, 1.3 eq) and dimethyl 4-[[tert-butyl(diphenyl)silyl]oxymethyl]-2-oxabicyclo[2.1.1]hexane-1,5-dicarboxylate (6 g, 12.80 mmol, 1 eq) in MeOH (300 mL) was added NaOH (768.17 mg, 19.21 mmol, 1.5 eq). The mixture was stirred at 25° C. for 12 h. LCMS showed starting material was consumed and the desired mass was detected. The reaction was concentrated under reduced pressure to give a residue. The residue was purified by flash silica gel chromatography (SiO₂, PE:EtOAc=5:1, Rf=0.35) to give compound methyl 1-(6-bromo-8-methyl-[1,2,4]triazolo[1,5-a]pyridin-2-yl)-4-[[tert-butyl(diphenyl)silyl]oxymethyl]-2-oxabicyclo[2.1.1]hexane-5-carboxylate (4.1 g, 52% yield) as a yellow gum.

LC-MS [ESI, M+1]: 620.3.

¹H NMR (400 MHz, CHLOROFORM-d) δ=8.58 (s, 1H), 7.71-7.64 (m, 4H), 7.48-7.36 (m, 7H), 4.19-4.12 (m, 1H), 4.10 (d, J=6.0 Hz, 1H), 4.07-4.02 (m, 1H), 3.97-3.92 (m, 1H), 3.59 (s, 3H), 3.34 (s, 1H), 2.67 (s, 3H), 2.33 (d, J=7.6 Hz, 1H), 2.02 (d, J=7.6 Hz, 1H), 1.09 (s, 9H).

### Step 5: methyl 1-(6-bromo-8-methyl-[1,2,4]triazolo[1,5-a]pyridin-2-yl)-4-(hydroxymethyl)-2-oxabicyclo[2.1.1]hexane-5-carboxylate

To a solution of methyl 1-(6-bromo-8-methyl-[1,2,4]triazolo[1,5-a]pyridin-2-yl)-4-[[tertbutyl(diphenyl)silyl]oxymethyl]-2-oxabicyclo[2.1.1]hexane-5-carboxylate (4.7 g, 7.57 mmol, 1 eq) in DMSO (20 mL) and THF (10 mL) was added CsF (1.73 g, 11.36 mmol, 1.5 eq). The mixture was stirred at 25° C. for 12 h. LCMS showed starting material was consumed and one main peak with desired mass was detected. The mixture was quenched with H₂O (100 mL) and extracted with EtOAc (50 mL×3), the organic phase was dried, filtered, and concentrated under pressure to give the product. The residue was purified by flash silica gel chromatography (SiO₂, DCM:MeOH=15:1, Rf=0.42). The crude product was further triturated with ACN at 25° C. for 15 min and the solid was filtered to give compound methyl 1-(6-bromo-8-methyl-[1,2,4]triazolo[1,5-a]pyridin-2-yl)-4-(hydroxymethyl)-2-oxabicyclo[2.1.1]hexane-5-carboxylate (2 g, 69% yield) as a white solid.

LC-MS [ESI, M+1]: 381.8.

¹H NMR (400 MHz, CHLOROFORM-d) δ=8.58 (s, 1H), 7.43 (s, 1H), 4.23 (d, J=6.4 Hz, 1H), 4.19-4.06 (m, 2H), 3.99 (d, J=6.4 Hz, 1H), 3.65 (s, 3H), 3.36 (s, 1H), 2.68 (s, 3H), 2.27 (d, J=7.6 Hz, 1H), 2.04 (d, J=6.8 Hz, 1H).

### Step 6: 1-(6-bromo-8-methyl-[1,2,4]triazolo[1,5-a]pyridin-2-yl)-5-(methoxycarbonyl)-2-oxabicyclo[2.1.1]hexane-4-carboxylic acid

To a solution of methyl 1-(6-bromo-8-methyl-[1,2,4]triazolo[1,5-a]pyridin-2-yl)-4-(hydroxymethyl)-2-oxabicyclo[2.1.1]hexane-5-carboxylate (3 g, 7.85 mmol, 1 eq) in ACN (10 mL), H₂O (10 mL) and acetone (10 mL) was added PIDA (6.57 g, 20.41 mmol, 2.6 eq) and TEMPO (493.72 mg, 3.14 mmol, 0.4 eq). The mixture was stirred at 50° C. for 3 h. LCMS showed starting material was consumed and one main peak with desired mass was detected. The mixture was quenched with H₂O (10 mL) and extracted with EtOAc (20 mL×3), the organic phase was dried, filtered, and concentrated under pressure to give the product. The crude product was further triturated with MTBE at 0° C. for 15 min and the solid was filtered to give compound 1-(6-bromo-8-methyl-[1,2,4]triazolo[1,5-a]pyridin-2-yl)-5-methoxycarbonyl-2-oxabicyclo[2.1.1]hexane4-carboxylic acid (2.6 g, 84% yield) as a white solid.

LC-MS [ESI, M+1]: 398.0

¹H NMR (400 MHz, CHLOROFORM-d) δ=8.60 (s, 1H), 7.45 (s, 1H), 4.51-4.35 (m, 1H), 4.25 (d, J=6.4 Hz, 1H), 3.78-3.73 (m, 1H), 3.71-3.60 (m, 3H), 2.73-2.62 (m, 4H), 2.40-2.30 (m, 1H).

### Step 7: 4-(tert-butyl) 5-methyl 1-(6-bromo-8-methyl-[1,2,4]triazolo[1,5-a]pyridin-2-yl)-2-oxabicyclo[2.1.1]hexane-4,5-dicarboxylate

To a solution of 1-(6-bromo-8-methyl-[1,2,4]triazolo[1,5-a]pyridin-2-yl)-5-methoxycarbonyl-2-oxabicyclo[2.1.1]hexane-4-carboxylic acid (3.5 g, 8.83 mmol, 1 eq) in t-BuOH (30 mL) was added Boc₂O (38.56 g, 176.68 mmol, 40.59 mL, 20 eq), DMAP (1.08 g, 8.83 mmol, 1 eq) and pyridine (13.98 g, 176.68 mmol, 20 eq). The mixture was stirred at 25° C. for 6 h. LCMS showed starting material was consumed and one main peak with desired mass was detected. The mixture was quenched with H₂O (15 mL) and extracted with EtOAc (20 mL×3), the organic phase was washed with saturated NaCl (20 mL×3), dried, filtered, and concentrated under pressure to give the product. The residue was purified by flash silica gel chromatography SiO₂, PE:EtOAc=3:1, Rf=0.42) to give compound O4-tert-butyl O5-methyl 1-(6-bromo-8-methyl-[1,2,4]triazolo[1,5-a]pyridin-2-yl)-2-oxabicyclo[2.1.1]hexane-4,5-dicarboxylate (2.3 g, 57% yield) as a brown gum.

LC-MS [ESI, M+1]: 455.2.

¹H NMR (400 MHz, CHLOROFORM-d) δ=8.49 (d, J=1.2 Hz, 1H), 7.33 (d, J=1.2 Hz, 1H), 4.27 (d, J=6.4 Hz, 1H), 4.08 (d, J=6.4 Hz, 1H), 3.56 (s, 3H), 3.53 (s, 1H), 2.58 (s, 3H), 2.49 (d, J=7.2 Hz, 1H), 2.18 (dd, J=7.2, 1.2 Hz, 1H), 1.45 (s, 9H).

### Step 8: 4-(tert-butyl) 5-methyl 1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4,5-dicarboxylate

A mixture of (3R)-3-methyl-3-phenyl-pyrrolidine (901.94 mg, 5.59 mmol, 1.1 eq), O4-tert-butyl O5-methyl 1-(6-bromo-8-methyl-[1,2,4]triazolo[1,5-a]pyridin-2-yl)-2-oxabicyclo[2.1.1]hexane-4,5-dicarboxylate (2.3 g, 5.09 mmol, 1 eq), Pd-PEPPSI-IHeptCl (445.20 mg, 457.66 μmol, 0.09 eq) and Cs₂CO₃ (3.31 g, 10.17 mmol, 2 eq) in dioxane (30 mL) was degassed and purged with N₂ for 3 times, and then the mixture was stirred at 100° C. for 10 h under N₂ atmosphere. LCMS showed starting material was consumed and one main peak with desired mass was detected. The mixture was filtered, and concentrated under pressure to give the product. The residue was purified by flash silica gel chromatography (SiO₂, PE:EtOAc=5:1, Rf=0.51) to give compound O4-tert-butyl O5-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (2.2 g, 81% yield) as a yellow solid.

LC-MS [ESI, M+1]: 533.2.

¹H NMR (400 MHz, CHLOROFORM-d) δ=7.68 (d, J=1.8 Hz, 1H), 7.41-7.32 (m, 4H), 7.28 (br s, 1H), 6.96 (s, 1H), 4.37 (d, J=6.4 Hz, 1H), 4.16 (d, J=6.4 Hz, 1H), 3.65 (s, 3H), 3.63-3.58 (m, 2H), 3.56-3.45 (m, 2H), 3.44-3.37 (m, 1H), 2.66 (s, 3H), 2.56 (d, J=7.2 Hz, 1H), 2.39 (td, J=8.0, 12.0 Hz, 1H), 2.31-2.23 (m, 2H), 1.54 (s, 9H), 1.48 (s, 3H).

### Step 9: 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylic acid

o a solution of O4-tert-butyl O5-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (20 mg, 37.55 μmol, 1 eq) in THF (0.5 mL) was added TMSOK (9.63 mg, 75.10 μmol, 2 eq). The mixture was stirred at 25° C. for 1 h. LCMS showed starting material was consumed and one main peak with desired mass was detected. The residue was added FA to adjust Ph=6 and then purified by prep-HPLC (column: Phenomenex luna C18 150*25 mm*10 um; mobile phase: [water(FA)—ACN]; gradient:36%-66% B over 10 min) to give 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylic acid (12 mg, 25.63 μmol, 68.27% yield, 98.8% purity) as yellow solid.

LC-MS [ESI, M+1]: 463.2.

¹H NMR (400 MHz, CHLOROFORM-d) δ=7.64 (s, 1H), 7.42-7.34 (m, 2H), 7.31 (br d, J=7.8 Hz, 2H), 7.27-7.23 (m, 1H), 7.08 (br s, 1H), 4.28 (br d, J=6.0 Hz, 1H), 4.21 (br d, J=6.0 Hz, 1H), 3.62 (br d, J=8.6 Hz, 1H), 3.57-3.47 (m, 2H), 3.45-3.37 (m, 2H), 2.68-2.51 (m, 4H), 2.47-2.35 (m, 2H), 2.33-2.22 (m, 1H), 1.47 (s, 3H).

### Example 74

### (2R,5S)-5-((5-(3-methyl-1H-1,2,4-triazol-5-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)carbamoyl)tetrahydrofuran-2-carboxylic acid (Compound 538)

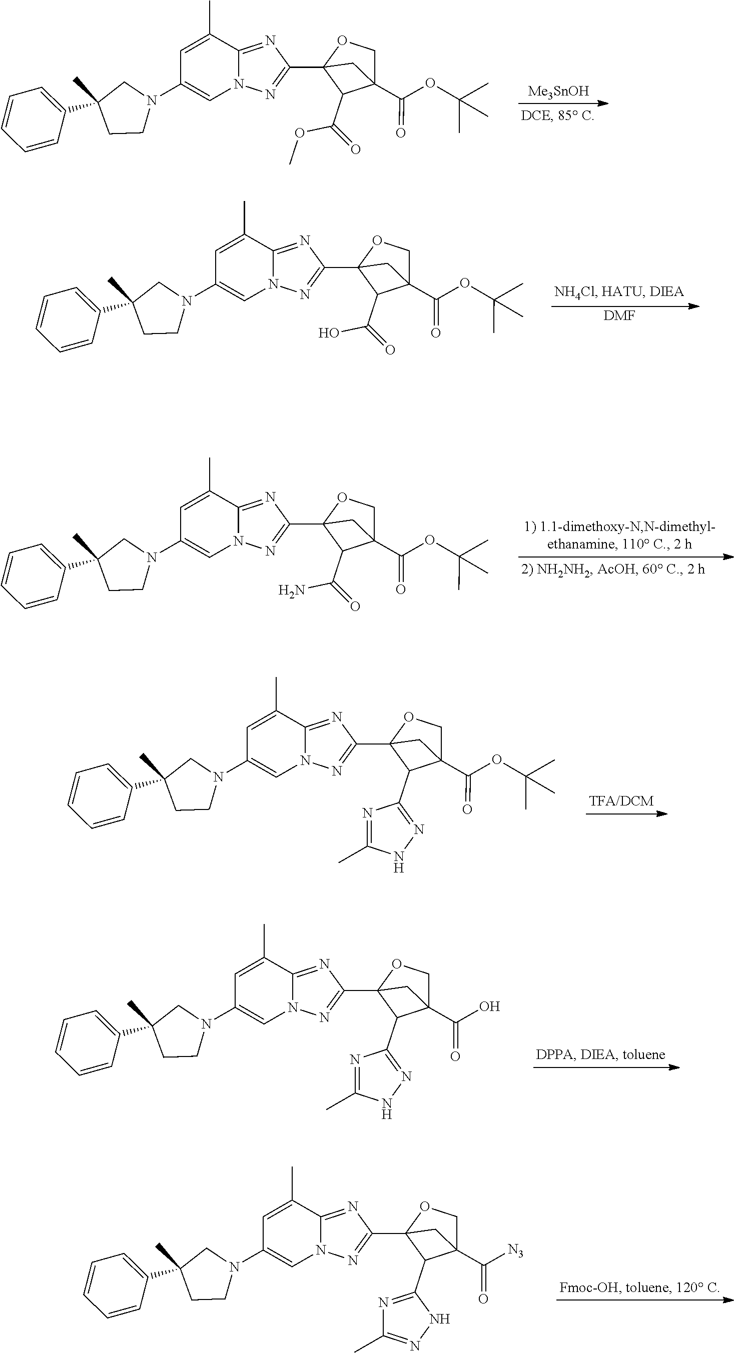

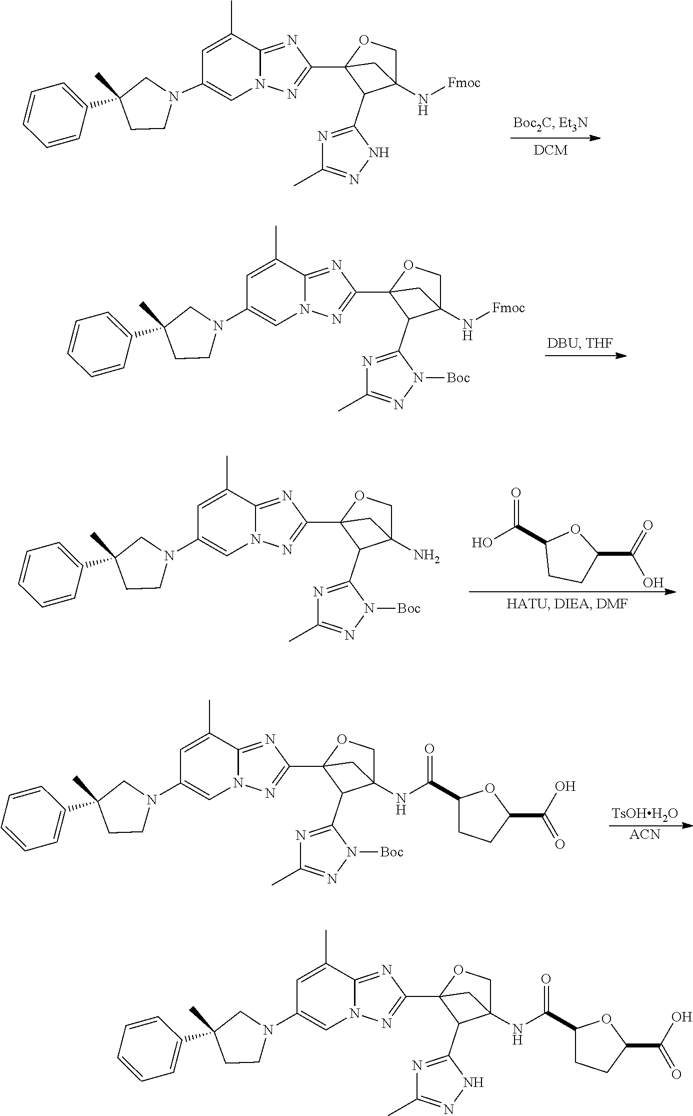

### Step 1: 4-(tert-butoxycarbonyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic acid

To a solution of O4-tert-butyl O5-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (600 mg, 1.13 mmol, 1 eq) in DCE (10 mL) was added hydroxy(trimethyl)stannane (1.02 g, 5.63 mmol, 5 eq). The mixture was stirred at 80° C. for 12 h. LCMS showed starting material was consumed and one main peak with desired mass was detected. The mixture was concentrated in vaco. The residue was purified by flash silica gel chromatography (SiO₂, PE:EtOAc=4:1, Rf=0.50) to give compound 4-tert-butoxycarbonyl-1-[8-methyl-6-[(3R)-3-methyl-3-phenyl-pyrrolidin-1-yl]-[1,2,4]triazolo[1,5-a]pyridin2-yl]-2-oxabicyclo[2.1.1]hexane-5-carboxylic acid (500 mg, 85% yield) as yellow solid.

LC-MS [ESI, M+1]: 519.3.

### Step 2: tert-butyl 5-carbamoyl-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylate

To a solution of 4-tert-butoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.42 g, 2.74 mmol, 1 eq) and NH₄Cl (732.33 mg, 13.69 mmol, 5 eq) in DMF (10 mL) was added HATU (1.56 g, 4.11 mmol, 1.5 eq) and DIEA (1.06 g, 8.21 mmol, 1.43 mL, 3 eq). The mixture was stirred at 25° C. for 1 h. LCMS showed starting material was consumed and one main peak with desired mass was detected. The mixture was filtered, and concentrated under pressure to give the product. The residue was purified by prep-HPLC purification (mobile phase: [water(FA)—ACN]; B %: 60%-70%, 6 min), the mixture was concentrated and lyophilized to give compound tert-butyl 5-carbamoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (1.02 g, 72% yield) as a green solid.

LC-MS [ESI, M+1]: 518.2.

¹H NMR (400 MHz, CHLOROFORM-d) δ=7.64 (d, J=1.6 Hz, 1H), 7.43-7.30 (m, 4H), 7.28 (s, 1H), 7.00 (s, 1H), 5.51 (br s, 1H), 4.28 (d, J=6.4 Hz, 1H), 4.12 (d, J=6.4 Hz, 1H), 3.61 (d, J=8.8 Hz, 1H), 3.50 (br t, J=9.2 Hz, 2H), 3.41 (br d, J=3.8 Hz, 1H), 3.38 (s, 1H), 2.68-2.56 (m, 3H), 2.46-2.34 (m, 3H), 2.33-2.22 (m, 1H), 1.53 (s, 8H), 1.48 (s, 3H).

### Step 3: tert-butyl 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylate

To a solution of tert-butyl 5-carbamoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (0.8 g, 1.55 mmol, 1 eq) in 1,1-dimethoxy-N,N-dimethylethanamine (24.14 g, 26.50 mL), the mixture was stirred at 110° C. for 2 h. Then the mixture was concentrated under reduced pressure to give a residue. To a solution of this residue in EtOH (10 mL) was added acetic acid (1.86 g, 30.91 mmol, 1.77 mL, 20 eq) and hydrazine;hydrate (910.24 mg, 15.46 mmol, 882.01 μL, 85% purity, 10 eq) at −10° C. And then the mixture was stirred at 25° C. for 1 h. LCMS showed starting material was consumed and one main peak with desired mass was detected. The mixture was quenched with water (15 mL) and extracted with ethyl acetate (10 mL×3), the combined organic phase was dried over anhydrous sodium sulfate, filtered and concentrated to give a residue. The residue was purified by flash silica gel chromatography (SiO₂, DCM:MeOH=10:1, Rf=0.32) to give compound tert-butyl 1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexane-4-carboxylate (805 mg, 93% yield) as a brown solid.

LC-MS [ESI, M+1]: 556.3.

### Step 4: 5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-4-carboxylic acid

To a solution of tert-butyl 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carboxylate (500 mg, 899.82 μmol, 1 eq) in DCM (5 mL) was added TFA (1 mL). The mixture was stirred at 25° C. for 1 h. LCMS showed starting material was consumed and one main peak with desired mass was detected. The mixture was concentrated and purified by reversed-phase HPLC (0.1% FA condition) to give 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carboxylic acid (440 mg, 880.77 μmol, 97.88% yield) as yellow solid

LC-MS [ESI, M+1]: 500.2.

¹H NMR (CHLOROFORM-d, 400 MHz) δ 7.67 (s, 1H), 7.4-7.4 (m, 2H), 7.3-7.4 (m, 3H), 7.04 (s, 1H), 4.17 (br d, 1H, J=6.4 Hz), 3.92 (br d, 1H, J=6.0 Hz), 3.82 (br s, 1H), 3.63 (br d, 1H, J=8.8 Hz), 3.5-3.6 (m, 2H), 3.4-3.5 (m, 1H), 2.77 (br d, 1H, J=6.0 Hz), 2.67 (s, 3H), 2.49 (s, 3H), 2.4-2.4 (m, 1H), 2.2-2.3 (m, 2H), 1.49 (s, 3H).

### Step 5: 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carbonyl azide

To a solution of 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carboxylic acid (200 mg, 400.35 μmol, 1 eq) in toluene (3 mL), THF (0.5 mL) was added DIEA (155.23 mg, 1.20 mmol, 209.20 μL, 3 eq), DPPA (165.26 mg, 600.52 μmol, 129.62 μL, 1.5 eq) at 25° C. The mixture was stirred at 25° C. for 3 h. LCMS showed starting material was consumed and one main peak with desired mass was detected. The mixture was quenched with water (10 mL) and extracted with ethyl acetate (5 mL×3), the combined organic phase was dried over anhydrous sodium sulfate, filtered and concentrated to give 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carbonyl azide (200 mg, crude) as yellow oil.

LC-MS [ESI, M+1]: 525.3.

### Step 6: 9H-fluoren-9-ylmethyl N-[1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexan-4-yl]carbamate

To a solution of 1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexane-4-carbonyl azide (200 mg, 381.26 μmol, 1 eq) in toluene (3 mL) was added 9H-fluoren-9-ylmethanol (299.28 mg, 1.53 mmol, 4 eq). The mixture was stirred at 110° C. for 0.5 h. LCMS showed starting material was consumed and one main peak with desired mass was detected. The mixture was concentrated and purified by reversed-phase HPLC (0.1% FA condition) to give 9H-fluoren-9-ylmethyl N-[1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexan-4-yl]carbamate (139 mg, 152.48 μmol, 39.99% yield, 76% purity) as yellow solid.

LC-MS [ESI, M+1]: 693.4.

¹H NMR (400 MHz, CHLOROFORM-d) δ=7.74-7.47 (m, 4H), 7.39-7.14 (m, 10H), 6.93 (br s, 1H), 4.35 (br s, 2H), 4.16 (br t, J=6.4 Hz, 1H), 4.08-3.94 (m, 1H), 3.80-3.64 (m, 1H), 3.62-3.49 (m, 2H), 3.48-3.38 (m, 2H), 3.33 (br dd, J=5.2, 8.8 Hz, 1H), 2.96-2.78 (m, 1H), 2.69-2.47 (m, 4H), 2.42-2.25 (m, 4H), 2.23-2.13 (m, 1H), 1.39 (s, 3H).

### Step 7: tert-butyl 5-[4-(9H-fluoren-9-ylmethoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate

To a solution of 9H-fluoren-9-ylmethyl N-[1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexan-4-yl]carbamate (110 mg, 158.77 μmol, 1 eq) in DCM (1.5 mL) was added Boc₂O (41.58 mg, 190.53 μmol, 43.77 μL, 1.2 eq), DMAP (969.85 g, 7.94 μmol, 0.05 eq), Et₃N (19.28 mg, 190.53 μmol, 26.52 μL, 1.2 eq). The mixture was stirred at 25° C. for 1 h. LCMS showed starting material was consumed and one main peak with desired mass was detected. The mixture was concentrated and purified by prep-TLC (SiO₂, PE:EtOAc=1:3, Rf=0.45) to give tert-butyl 5-[4-(9H-fluoren-9-ylmethoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate (70 mg, 88.28 μmol, 55.6% yield) as yellow solid.

LC-MS [ESI, M+1]: 793.5.

### Step 8: tert-butyl 5-[4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate

To a solution of tert-butyl 5-[4-(9H-fluoren-9-ylmethoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate (50 mg, 63.06 μmol, 1 eq) in ACN (0.2 mL) was added DBU (0.1 M, 315.29 μL, 0.5 eq). The mixture was stirred at 25° C. for 0.5 h. LCMS showed starting material was consumed and one main peak with desired mass was detected. The mixture was filtered and purified by reversed-phase HPLC (0.1% NH₃·H₂O) to give tert-butyl 5-[4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate (20 mg, 35.05 μmol, 55.6% yield) as yellow solid.

LC-MS [ESI, M+1]: 571.4.

### Step 9: 5-[[5-(2-tert-butoxycarbonyl-5-methyl-1,2,4-triazol-3-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamoyl]tetrahydrofuran-2-carboxylic acid

To a solution of tert-butyl 5-[4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-3-methyl-1,2,4-triazole-1-carboxylate (30 mg, 52.57 μmol, 1 eq), tetrahydrofuran-2,5-dicarboxylic acid (42.09 mg, 262.84 μmol, 5 eq) in DMF (0.5 mL) was added DIEA (13.59 mg, 105.14 μmol, 18.31 μL, 2 eq), T4P (37.88 mg, 105.14 μmol, 2 eq). The mixture was stirred at 25° C. for 1 h. LCMS showed starting material was consumed and one main peak with desired mass was detected. The crude product was purified by reversed-phase HPLC (neutral) to give 5-[[5-(2-tert-butoxycarbonyl-5-methyl-1,2,4-triazol-3-yl)-1-[8-methyl-6-[(3R)-3-methyl-3-phenyl-pyrrolidin-1-yl]-[1,2,4]triazolo[1, 5-a]pyridin-2-yl]-2-oxabicyclo[2.1.1]hexan-4-yl]carbamoyl]tetrahydrofuran-2-carboxylic acid (20 mg, 28.06 μmol, 53.38% yield) as yellow solid.

LC-MS [ESI, M+1]: 713.4.

### Step 10: 5-[[1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexan-4-yl]carbamoyl]tetrahydrofuran-2-carboxylic acid

To a solution of 5-[[5-(2-tert-butoxycarbonyl-5-methyl-1,2,4-triazol-3-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamoyl]tetrahydrofuran-2-carboxylic acid (20 mg, 28.06 μmol, 1 eq) in ACN (0.5 mL) was added 4-methylbenzenesulfonic acid;hydrate (1 M, 112.23 μL, 4 eq). The mixture was stirred at 25° C. for 1 h. LCMS showed starting material was consumed and one main peak with desired mass was detected. The mixture was added NaHCO₃ to adjust pH=7, then filtered. The residue was purified by prep-HPLC (column: CD24-WePure Biotech XPT C18 150*25*7 um; mobile phase: [H₂O (10 mM NH₄HCO₃)—ACN]; gradient:22%-52% B over 10.0 min) to give 5-[[1-[8-methyl-6-[(3R)-3-methyl-3-phenyl-pyrrolidin-1-yl]-[1,2,4]triazolo[1,5-a]pyridin-2-yl]-5-(3-methyl-1H-1,2,4-triazol-5-yl)-2-oxabicyclo[2.1.1]hexan-4-yl]carbamoyl]tetrahydrofuran-2-carboxylic acid (7.13 mg, 11.14 μmol, 39.7% yield, 95.7% purity) as white solid.

LC-MS [ESI, M+1]: 613.4.

¹H NMR (400 MHz, METHANOL-d4) δ=7.74 (s, 1H), 7.43-7.29 (m, 4H), 7.26-7.14 (m, 2H), 4.62-4.47 (m, 2H), 4.31-4.18 (m, 2H), 4.10 (dd, J=5.8, 11.2 Hz, 1H), 3.62-3.49 (m, 3H), 3.39 (td, J=4.2, 8.6 Hz, 1H), 3.07-2.94 (m, 1H), 2.58 (s, 3H), 2.42-2.23 (m, 8H), 2.22-2.00 (m, 2H), 1.43 (s, 3H).

| Compound | | |
|---|---|---|
| Number | Synthesis | LC/MS |
| 539 | Similar to Example 74 | m/z 485.3 [M + H]⁺ |
| 540 | Similar to Example 74 | m/z 527.2 [M + H]⁺ |
| 541 | Similar to Example 74 | m/z 514.5 [M + H]⁺ |
| 542 | Similar to Example 74 | m/z 514.2 [M + H]⁺ |

### Example 75

### N-((1R,4R,5R)-5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)acetamide (Compound 543)

### N-((1S,4S,5S)-5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)acetamide (Compound 544)

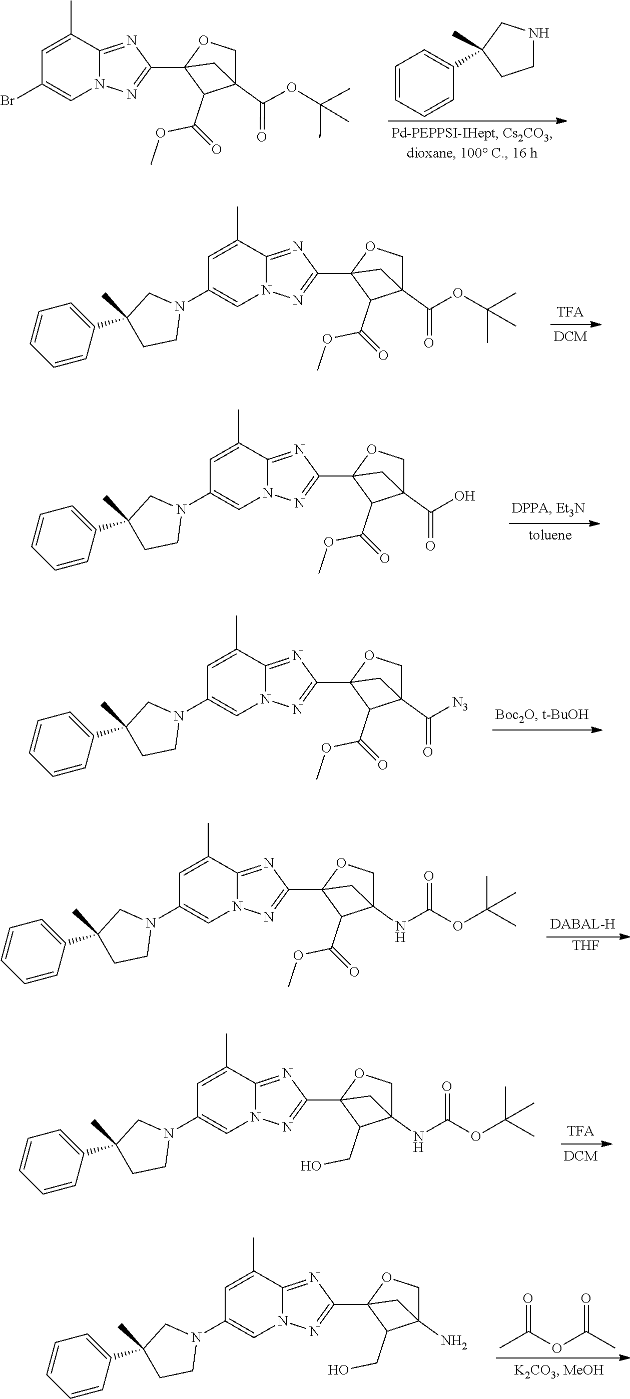

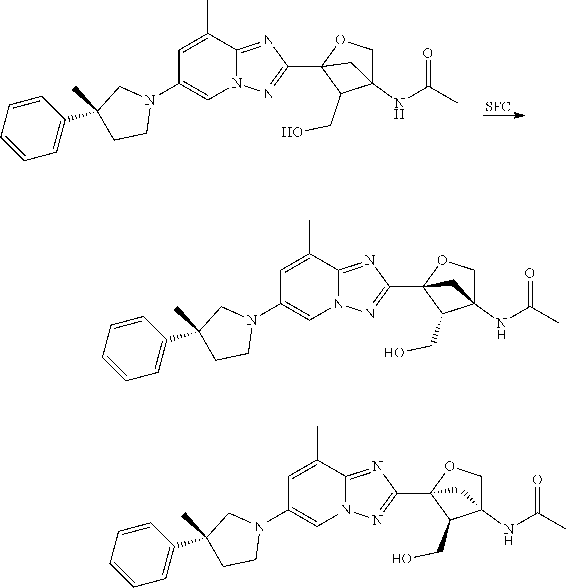

### Step 1: O4-tert-butyl O5-methyl 1-[8-methyl-6-1(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate

To an 8 mL vial equipped with a magnetic stir bar was added O4-tert-butyl 05-methyl 1-(6-bromo-8-methyl-[1,2,4]triazolo[1,5-a]pyridin-2-yl)-2-oxabicyclo[2.1.1]hexane-4,5-dicarboxylate (2.5 g, 5.53 mmol, 1 eq), (3R)-3-methyl-3-phenyl-pyrrolidine (1.07 g, 6.63 mmol, 1.2 eq), 1,3-bis[2,6-bis(1-propylbutyl)phenyl]-4,5-dichloro-2H-imidazol-1-ium-2-ide;3-chloropyridine;dichloropalladium (376.38 mg, 386.91 μmol, 0.07 eq), Cs₂CO₃ (4.50 g, 13.82 mmol, 2.5 eq). The vial was evacuated and backfilled with nitrogen three times. dioxane (30 mL) were added and reaction was stirred at 100° C. for 12 under N₂ atmosphere. LCMS showed starting material was consumed and one main peak with desired mass was detected. The mixture was filtered and concentrated under reduced pressure. The residue was purified by flash silica gel chromatography (SiO₂, PE:EtOAc=5:1, Rf=0.51) to give O4-tert-butyl O5-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (2.6 g, 4.88 mmol, 88.3% yield) as yellow solid.

LC-MS [ESI, M+1]: 533.3.

¹H NMR (400 MHz, CHLOROFORM-d) δ=7.68 (d, J=1.6 Hz, 1H), 7.41-7.32 (m, 4H), 7.28 (br s, 1H), 6.96 (s, 1H), 4.37 (d, J=6.4 Hz, 1H), 4.16 (d, J=6.4 Hz, 1H), 3.65 (s, 3H), 3.63-3.58 (m, 2H), 3.56-3.45 (m, 2H), 3.44-3.37 (m, 1H), 2.66 (s, 3H), 2.56 (d, J=7.2 Hz, 1H), 2.39 (td, J=8.0, 12.0 Hz, 1H), 2.31-2.23 (m, 2H), 1.54 (s, 9H), 1.48 (s, 3H).

### Step 2: 5-methoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid

A solution of O4-tert-butyl O5-methyl 1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4,5-dicarboxylate (2.6 g, 4.88 mmol, 1 eq) in DCM (20 mL), TFA (4 mL) was stirred at 25° C. for 12 h. LCMS showed starting material was consumed and one main peak with desired mass was detected. The mixture was concentrated and purified by reversed-phase HPLC (0.1% FA condition) to give 5-methoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (2 g, 4.20 mmol, 85.98% yield) as yellow solid.

LC-MS [ESI, M+1]: 477.3.

¹H NMR (400 MHz, CHLOROFORM-d) δ=7.70 (s, 1H), 7.42-7.36 (m, 2H), 7.35-7.31 (m, 2H), 7.28 (br s, 1H), 7.01 (s, 1H), 4.42 (d, J=6.4 Hz, 1H), 4.25 (d, J=6.4 Hz, 2H), 3.85 (s, 1H), 3.67 (s, 3H), 3.62 (d, J=8.8 Hz, 1H), 3.57-3.47 (m, 2H), 3.41 (dt, J=3.6, 8.8 Hz, 1H), 2.76 (d, J=7.4 Hz, 1H), 2.68 (s, 3H), 2.45-2.32 (m, 2H), 2.29 (br dd, J=4.0, 7.4 Hz, 1H), 1.48 (s, 3H).

### Step 3: methyl 4-carbonazidoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

To a solution of 5-methoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (500 mg, 1.05 mmol, 1 eq) in toluene (5 mL) was added DIEA (406.83 mg, 3.15 mmol, 548.29 μL, 3 eq), DPPA (433.14 mg, 1.57 mmol, 1.5 eq). The mixture was stirred at 25° C. for 1 h. LCMS showed starting material was consumed and one main peak with desired mass was detected. The mixture was quenched with water (20 mL) and extracted with ethyl acetate (15 mL×3), the combined organic phase was dried over anhydrous sodium sulfate, filtered and concentrated to give methyl 4-carbonazidoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (500 mg, 996.94 μmol, 95.0% yield) as yellow oil.

LC-MS [ESI, M+1]: 502.2.

### Step 4: methyl 4-(tert-butoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

A solution of methyl 4-carbonazidoyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (500 mg, 996.94 μmol, 1 eq), Boc₂O (1.09 g, 4.98 mmol, 1.15 mL, 5 eq), t-BuOH (15 mL) was stirred at 100° C. for 1 h. LCMS showed starting material was consumed and one main peak with desired mass was detected. The mixture was concentrated and purified by flash silica gel chromatography (SiO₂, PE:EtOAc=1:1, Rf=0.28) to give methyl 4-(tert-butoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (170 mg, 310.42 μmol, 31.1% yield) as yellow solid.

LC-MS [ESI, M+1]: 548.4.

¹H NMR (400 MHz, CHLOROFORM-d) δ=7.61 (s, 1H), 7.35-7.28 (m, 2H), 7.27-7.18 (m, 3H), 6.89 (s, 1H), 5.45 (s, 1H), 4.12-4.00 (m, 3H), 3.59 (s, 3H), 3.54 (br d, J=8.8 Hz, 1H), 3.50-3.39 (m, 2H), 3.33 (dt, J=3.6, 8.4 Hz, 1H), 2.80-2.63 (m, 1H), 2.58 (s, 3H), 2.41-2.27 (m, 2H), 2.24-2.14 (m, 1H), 1.48-1.37 (m, 12H).

### Step 5: tert-butyl N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate

To give methyl 4-(tert-butoxycarbonylamino)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (100 mg, 182.60 μmol, 1 eq) in THF (1 mL) was added DIBAL-H (1 M, 547.80 μL, 3 eq) at −78° C. The mixture was stirred at −78° C. for 1 h. LCMS showed starting material was consumed and several peaks with desired mass was detected. The mixture was quenched with water (3 mL) and extracted with ethyl acetate (3 mL×3), the combined organic phase was dried over anhydrous sodium sulfate, filtered and concentrated to give a residue. The residue was purified by prep-TLC(SiO₂, PE:EtOAc=1:3, Rf=0.38) to give tert-butyl N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate (40 mg, 76.98 μmol, 42.2% yield) as yellow solid.

LC-MS [ESI, M+1]: 520.4.

### Step 6: [4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methanol

To a solution of tert-butyl N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]carbamate (60 mg, 115.47 μmol, 1 eq) in DCM (0.2 mL), ACN (0.4 mL) was added 4-methylbenzenesulfonic acid;hydrate (43.93 mg, 230.93 μmol, 2 eq). The mixture was stirred at 25° C. for 1 h. LCMS showed starting material was consumed and one main peak with desired mass was detected. The mixture was added NaHCO₃ to adjust pH=7, then filtered. The residue was purified by prep-HPLC (column: CD24-WePure Biotech XPT C18 150*25*7 um;mobile phase: [H₂O (10 mM NH₄HCO₃)—ACN]; gradient:28%-58% B over 10.0 min) to give [4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methanol (21 mg, 50.06 μmol, 43.3% yield) as white solid.

LC-MS [ESI, M+1]: 420.2.

¹H NMR (400 MHz, METHANOL-d4) δ=7.68 (d, J=1.6 Hz, 1H), 7.42-7.29 (m, 4H), 7.27-7.11 (m, 2H), 3.82 (dd, J=7.2, 11.2 Hz, 1H), 3.75-3.65 (m, 3H), 3.57-3.46 (m, 3H), 3.40-3.34 (m, 1H), 2.54 (s, 3H), 2.41 (dd, J=6.4, 7.2 Hz, 1H), 2.37-2.22 (m, 2H), 2.21-2.11 (m, 2H), 1.42 (s, 3H).

### Step 7: N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide

To a solution of [4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methanol (100 mg, 238.37 μmol, 1 eq) in MeOH (2 mL) was added K₂CO₃ (65.89 mg, 476.74 μmol, 2 eq) and acetyl acetate (48.67 mg, 476.74 μmol, 44.77 μL, 2 eq). The mixture was stirred at 25° C. for 1 h. LCMS showed starting material was consumed and one main peak with desired mass was detected. The mixture was added FA to adjust pH=4. The residue was purified by prep-HPLC (column: CD24-WePure Biotech XPT C18 150*25*7 um; mobile phase: [H₂O (0.225% FA)—ACN]; gradient:28%-58% B over 11.0 min) to give N-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide (90 mg, 194.99 μmol, 81.8% yield) as white solid.

LC-MS [ESI, M+1]: 462.1.

¹H NMR (400 MHz, DMSO-d₆) δ=8.65 (s, 1H), 7.89 (s, 1H), 7.43-7.32 (m, 4H), 7.27-7.19 (m, 2H), 4.49 (br s, 1H), 3.85-3.79 (m, 1H), 3.79-3.74 (m, 1H), 3.69-3.61 (m, 1H), 3.61-3.55 (m, 1H), 3.54-3.45 (m, 3H), 3.39-3.36 (m, 1H), 2.57 (t, J=6.4 Hz, 1H), 2.50 (br s, 3H), 2.33-2.22 (m, 3H), 2.17 (d, J=7.2 Hz, 1H), 1.86 (s, 3H), 1.36 (s, 3H).

### Step 8: N-[(1R,4R,5R)-5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide

The residue was purified by prep-SFC (column: SFC-IK-30-DAICEL CHIRAL IK (250 mm*30 mm, 10 um);mobile phase: [C02-MeOH:ACN=7:3 (0.1% NH₃·H₂O))]; B %: 53%, isocratic elution mode). Then the product was further purified by prep-HPLC (column: CD01-Phenomenex luna C18 150*25*10 um; mobile phase: [H₂O (0.225% FA)—ACN]; gradient:30%-60% B over 10.0 min) to give N-[(1R,4R,5R)-5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide (17.15 mg, 37.16 μmol, 21.4% yield, 100% purity) as white solid.

LC-MS [ESI, M+1]: 462.3.

¹H NMR (400 MHz, DMSO-d₆) δ=8.65 (s, 1H), 7.89 (s, 1H), 7.43-7.30 (m, 4H), 7.27-7.20 (m, 2H), 4.55-4.43 (m, 1H), 3.83-3.73 (m, 2H), 3.68-3.59 (m, 1H), 3.59-3.55 (m, 1H), 3.54-3.43 (m, 3H), 3.39-3.35 (m, 1H), 2.56 (t, J=6.4 z, 1H), 2.49 (br s, 3H), 2.31-2.20 (m, 3H), 2.16 (d, J=7.2 Hz, 1H), 1.85 (s, 3H), 1.35 (s, 3H).

### Step 8: [(1S,4S,5S)-5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide

The residue was purified by prep-SFC (column: SFC-IK-30-DAICEL CHIRAL IK (250 mm*30 mm, 10 um);mobile phase: [C02-MeOH:ACN=7:3 (0.1% NH₃·H₂O))]; B %: 53%, isocratic elution mode). Then the product was further purified by prep-HPLC (column: CD01-Phenomenex luna C18 150*25*10 um; mobile phase: [H₂O (0.225% FA)—ACN]; gradient:30%-60% B over 10.0 min) to give N-[(1S,4S,5S)-5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]acetamide (25.32 mg, 54.25 μmol, 31.3% yield, 98.9% purity) as white solid.

LC-MS [ESI, M+1]: 462.4.

¹H NMR (400 MHz, DMSO-d₆) δ=8.65 (s, 1H), 7.89 (d, J=1.6 Hz, 1H), 7.42-7.31 (m, 4H), 7.28-7.19 (m, 2H), 4.49 (br s, 1H), 3.86-3.72 (m, 2H), 3.62 (br d, J=7.2 Hz, 1H), 3.59-3.54 (m, 1H), 3.53-3.43 (m, 3H), 3.40-3.36 (m, 1H), 2.56 (t, J=6.4 Hz, 1H), 2.49 (br s, 3H), 2.32-2.22 (m, 3H), 2.16 (d, J=7.2 Hz, 1H), 1.85 (s, 3H), 1.35 (s, 3H).

### Example 76

### 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (Compound 545)

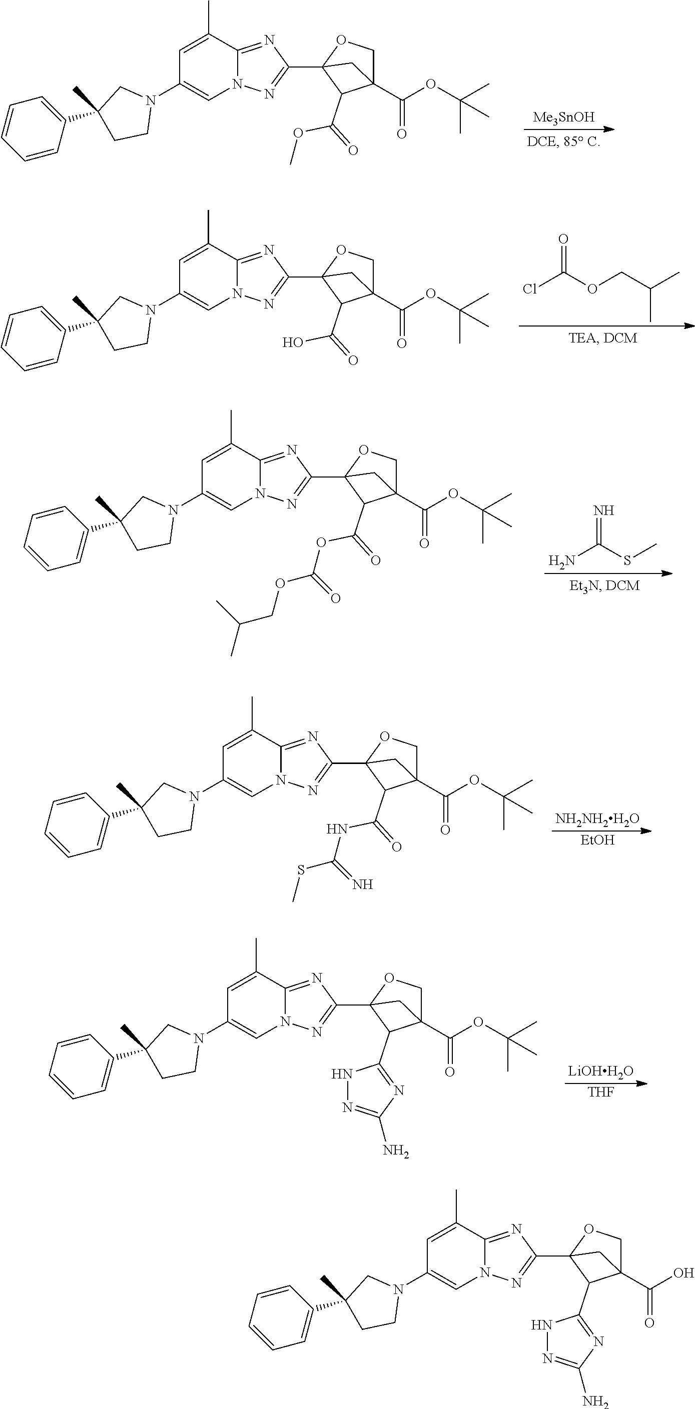

### Step 1: 4-tert-butoxycarbonyl-1--8-methyl-6-[(3R)-3-methyl-3-phenyl-pyrrolidin-1-yl]-11,2,4]triazolo[1,5-a]pyridin-2-yl]-2-oxabicyclo[2.1.1]hexane-5-carboxylic acid

To a solution of O4-tert-butyl O5-methyl 1-[8-methyl-6-[(3R)-3-methyl-3-phenyl-pyrrolidin-1-yl]-[[1,2,4]triazolo[1, 5-a]pyridin-2-yl]-2-oxabicyclo[2.1.1]hexane-4, 5-dicarboxylate (720 mg, 1.35 mmol, 1 eq) in DCE (10 mL) was added hydroxy(trimethyl)stannane (440 mg, 2.43 mmol, 1.8 eq).The mixture was stirred at 80° C. for 12 h. LCMS showed starting material was consumed and one main peak with desired mass was detected. The mixture was quenched with H₂O (10 mL) and extracted with ethyl acetate (10 mL×3), the combined organic phase was dried over anhydrous sodium sulfate, filtered and concentrated to give compound 4-tert-butoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (720 mg, 85% yield) as yellow solid.

LC-MS [ESI, M+1]: 519.2.

### Step 2: 4-(tert-butoxycarbonyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic (isobutyl carbonic) anhydride

To a solution of 4-tert-butoxycarbonyl-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (700 mg, 1.35 mmol, 1 eq) in DCM (5 mL) was added Et₃N (409.75 mg, 4.05 mmol, 563.62 μL, 3 eq) and isobutyl carbonochloridate (202.78 mg, 1.48 mmol, 194.24 μL, 1.1 eq) at 0° C. The mixture was stirred at 0° C. for 0.5 h. LCMS showed starting material was consumed and one main peak with desired mass was detected. The mixture was quenched with H₂O (10 mL) and extracted with ethyl acetate (10 mL×3), the combined organic phase was dried over anhydrous sodium sulfate, filtered and concentrated to give compound 4-(tert-butoxycarbonyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic (isobutyl carbonic) anhydride (835 mg, crude) as yellow solid.

LC-MS [ESI, M+1]: 619.6.

### Step 3: tert-butyl 1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexane-4-carboxylate

To a solution of 4-(tert-butoxycarbonyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic (isobutyl carbonic) anhydride (830 mg, 1.34 mmol, 1 eq) in DCM (5 mL) was added Et₃N (407.23 mg, 4.02 mmol, 560.15 μL, 3 eq) and 2-methylisothiourea (725.59 mg, 8.05 mmol, 6 eq). The mixture was stirred at 25° C. for 1 h. LCMS showed starting material was consumed and one main peak with desired mass was detected. The mixture was quenched with H₂O (10 mL) and extracted with ethyl acetate (10 mL×3), the combined organic phase was dried over anhydrous sodium sulfate, filtered and concentrated to give compound tert-butyl 1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexane-4-carboxylate (750 mg, crude) as yellow gum.

LC-MS [ESI, M+1]: 591.4.

### Step 4: tert-butyl 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate

To a solution of tert-butyl 1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexane-4-carboxylate (750 mg, 1.27 mmol, 1 eq) in EtOH (5 mL) was added NH₂NH₂·H₂O (747.73 mg, 12.70 mmol, 14.94 μL, 85% purity, 10 eq).The mixture was stirred at 60° C. for 1 h. LCMS showed starting material was consumed and one main peak with desired mass was detected. The reaction mixture was quenched by H₂O (5 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.35) to give compound tert-butyl 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (438 mg, 62% yield) as yellow solid.

LC-MS [ESI, M+1]: 557.3.

¹H NMR (400 MHz, CHLOROFORM-d) δ=7.64 (br s, 1H), 7.37 (br d, J=7.2 Hz, 2H), 7.34-7.30 (m, 2H), 7.27-7.24 (m, 1H), 6.99 (br s, 1H), 6.83 (br dd, J=3.6 7.2 Hz, 2H), 4.22 (br d, J=6.0 Hz, 1H), 4.18-4.05 (m, 3H), 4.03-3.97 (m, 1H), 3.61 (br d, J=8.8 Hz, 2H), 2.68-2.60 (m, 3H), 2.44-2.35 (m, 2H), 2.33-2.24 (m, 2H), 1.60-1.50 (m, 9H), 1.47 (s, 4H).

### Step 5: 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid

To a solution of tert-butyl 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylate (20 mg, 35.93 μmol, 1 eq) in THF (1 mL) was added LiOH·H₂O (3.02 mg, 71.86 μmol, 2 eq) and H₂O (0.3 mL). The mixture was stirred at 25° C. for 1 h. LCMS showed starting material was consumed and the desired mass was detected. The reaction was concentrated under reduced pressure to give a residue. The residue was purified by prep-HPLC purification (mobile phase: [water(FA)—ACN]; B %: 22%-52%, 2 min), the mixture was concentrated and lyophilized to give compound 5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-4-carboxylic acid (2.53 mg, 14% yield) as a white solid.

LC-MS [ESI, M+1]: 501.2.

¹H NMR (400 MHz, CHLOROFORM-d) δ=7.63 (br s, 1H), 7.43-7.33 (m, 2H), 7.31 (br d, J=6.4 Hz, 2H), 7.26-7.23 (m, 1H), 7.01 (br d, J=11.2 Hz, 1H), 4.82 (br s, 1H), 4.23-4.10 (m, 1H), 3.94 (br d, J=4.0 Hz, 1H), 3.72 (br s, 1H), 3.60 (br s, 1H), 3.52-3.28 (m, 3H), 2.73 (br dd, J=1.2, 4.8 Hz, 1H), 2.66-2.57 (m, 3H), 2.41 (br s, 1H), 2.37 (br d, J=7.2 Hz, 1H), 2.30-2.24 (m, 1H), 1.46 (br d, J=2.8 Hz, 3H).

### Example 77

### 1-(5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)-2-oxopiperidine-4-carboxylic acid (Compound 546)

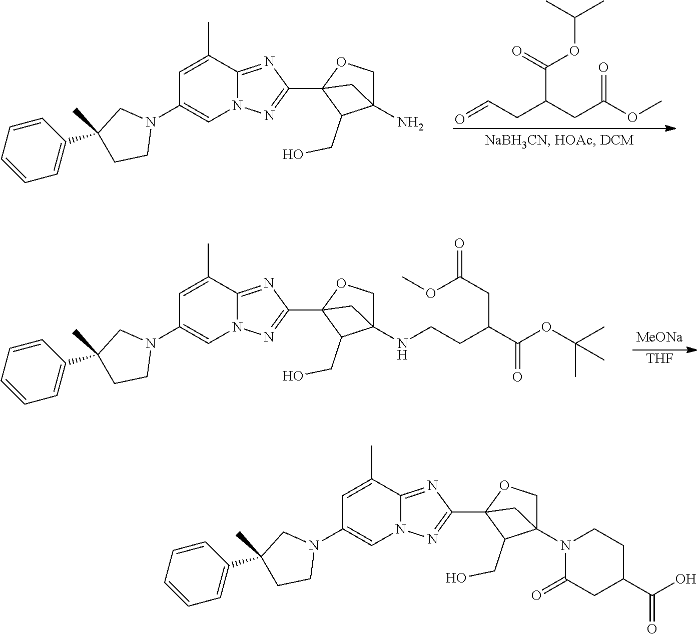 [SMILES: "MeONa" ; Cc1cc(N2CC[C@](C)(c3ccccc3)C2)cn2nc(C34CC(N)(CO3)C4CO)nc12 ; COC(=O)CC(CC=O)C(=O)OC(C)C ; COC(=O)CC(CCNC12COC(c3nc4c(C)cc(N5CC[C@](C)(c6ccccc6)C5)cn4n3)(C1)C2CO)C(=O)OC(C)(C)C ; Cc1cc(N2CC[C@](C)(c3ccccc3)C2)cn2nc(C34CC(N5CCC(C(=O)O)CC5=O)(CO3)C4CO)nc12 ; "THF"]

### Step 1: O1-tert-butyl O4-methyl 2-[2-[[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]ethyl]butanedioate

To a solution of [4-amino-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methanol (24 mg, 57.21 μmol, 1 eq), O1-tert-butyl O4-methyl 2-(2-oxoethyl)butanedioate (26.35 mg, 114.42 μmol, 2 eq) in MeOH (1 mL) was added HOAc (3.44 mg, 57.21 μmol, 3.27 μL, 1 eq) at 0° C., then NaBH₃CN (7.19 mg, 114.42 μmol, 2 eq) was added, the mixture was stirred at 0° C. for 0.5 h. LCMS showed starting material was consumed and one main peak with desired mass was detected. The mixture was quenched with water(0.1 mL), then filtered. The crude product was purified by reversed-phase HPLC (0.1% FA condition) to give O1-tert-butyl O4-methyl 2-[2-[[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]ethyl]butanedioate (21 mg, 33.13 μmol, 57.92% yield) as yellow oil.

LC-MS [ESI, M+1]: 634.4.

### Step 2: 1-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-2-oxo-piperidine-4-carboxylic acid

To a solution of O1-tert-butyl O4-methyl 2-[2-[[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]amino]ethyl]butanedioate (20 mg, 31.56 μmol, 1 eq) in THF (0.5 mL) was added NaOMe (4 M, 15.78 μL, 2 eq). The mixture was stirred at 25° C. for 1 h. LCMS showed starting material was consumed and one main peak with desired mass was detected. The mixture added FA to adjust pH=7. The residue was purified by prep-HPLC (column: CD04-Welch Utimate C18 150*25*7 um; mobile phase: [H₂O (0.225% FA)—ACN]; gradient:26%-56% B over 15.0 min) to give 1-[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]-2-oxo-piperidine-4-carboxylic acid (7.22 mg, 13.03 μmol, 41.30.1% yield, 98.5% purity) as white solid.

LC-MS [ESI, M+1]: 546.3.

¹H NMR (400 MHz, METHANOL-d4) δ=7.75 (s, 1H), 7.43-7.29 (m, 4H), 7.26-7.18 (m, 2H), 4.15 (t, J=6.0 Hz, 1H), 3.96 (dd, J=5.6, 9.2 Hz, 1H), 3.86-3.78 (m, 2H), 3.65-3.48 (m, 5H), 3.41 (dt, J=3.6, 8.8 Hz, 1H), 2.90 (br d, J=5.6 Hz, 1H), 2.72 (td, J=3.2, 6.0 Hz, 1H), 2.60 (br d, J=7.2 Hz, 2H), 2.57 (s, 3H), 2.51-2.42 (m, 2H), 2.40-2.28 (m, 2H), 2.27-2.18 (m, 1H), 2.08-1.94 (m, 1H), 1.44 (s, 3H).

### Example 78

### 4-[[5-[3-(ethylsulfonylamino)-1H-1,2,4-triazol-5-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methylamino]-2,2-dimethyl-4-oxo-butanoic acid (Compound 547)

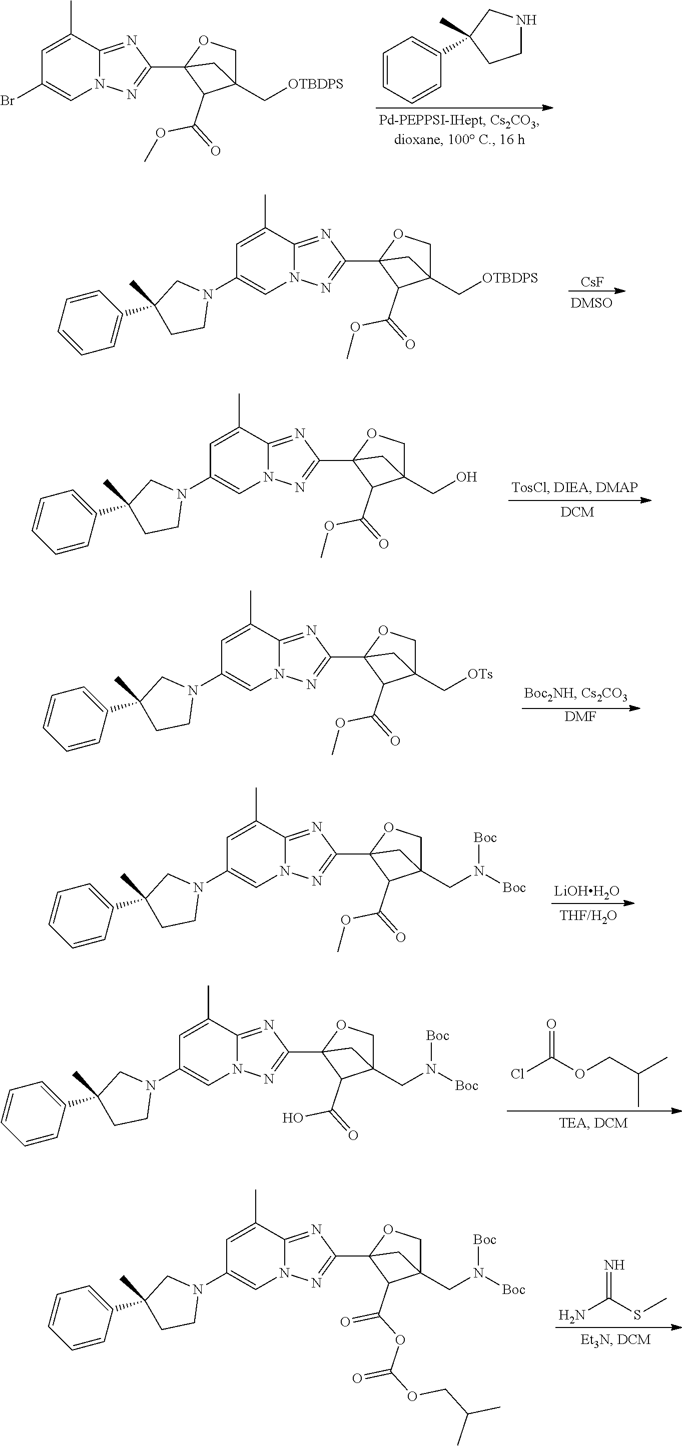

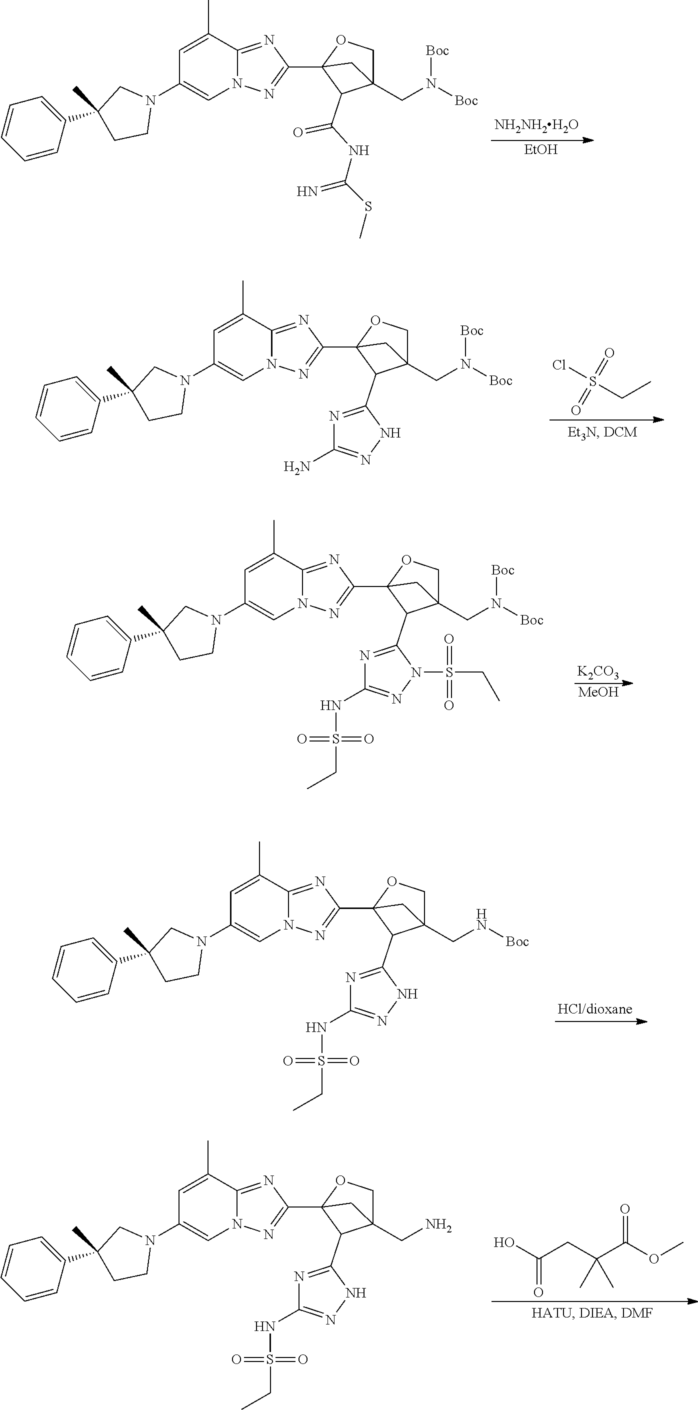

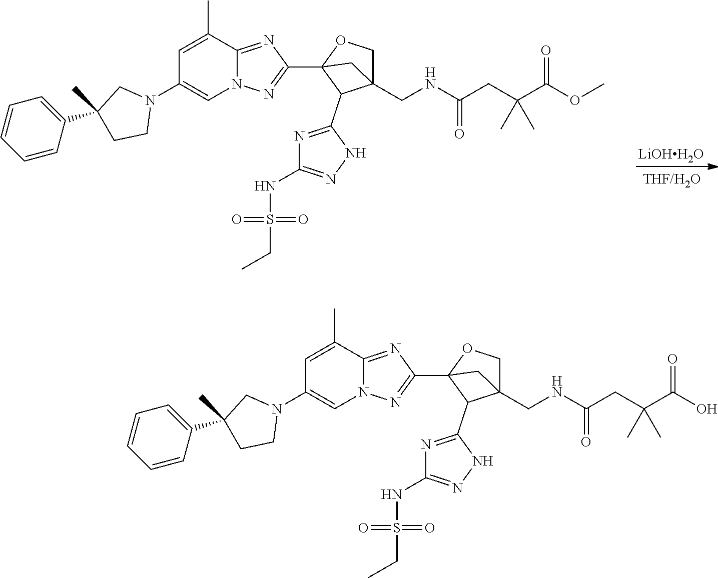 [SMILES: CCS(=O)(=O)Nc1n[nH]c(C2C3(CNC(=O)CC(C)(C)C(=O)OC)COC2(c2nc4c(C)cc(N5CC[C@](C)(c6ccccc6)C5)cn4n2)C3)n1 ; "LiOH·H2O" ; CCS(=O)(=O)Nc1n[nH]c(C2C3(CNC(=O)CC(C)(C)C(=O)O)COC2(c2nc4c(C)cc(N5CC[C@](C)(c6ccccc6)C5)cn4n2)C3)n1]

### Step 1: methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

A mixture of methyl 1-(6-bromo-8-methyl-[1,2,4]triazolo[1,5-a]pyridin-2-yl)-4-[[tertbutyl (diphenyl) silyl]oxymethyl]-2-oxabicyclo[2.1.1]hexane-5-carboxylate (10 g, 16.11 mmol, 1 eq), (3R)-3-methyl 3-phenyl-pyrrolidine (2.86 g, 17.72 mmol, 1.1 eq), dicesium;carbonate (15.75 g, 48.34 mmol, 3 eq) and 1,3-bis[2,6-bis(1-propylbutyl)phenyl]-4,5-dichloro-2H-imidazol-1-ium-2-ide;3-chloropyridine;dichloropalladium (470.24 mg, 483.40 μmol, 0.03 eq) in 1,4-dioxane (200 mL) was degassed and purged with N₂ for 3 times, and then the mixture was stirred at 100° C. for 12 h under N₂ atmosphere. LCMS showed starting material was consumed and the desired mass was detected. The reaction was filtered and the filtered concentrated under reduced pressure to give a residue. The residue was purified by flash silica gel chromatography (SiO₂, PE:EtOAc=5:1, Rf=0.3) to give compound methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (8.5 g, 75.26% yield) as a yellow oil.

LC-MS [ESI, M+1]:701.4

¹H NMR (400 MHz, CHLOROFORM-d) δ=7.60 (dd, J=1.2, 6.4 Hz, 5H), 7.44-7.07 (m, 11H), 6.86 (d, J=0.8 Hz, 1H), 4.12-4.01 (m, 3H), 4.00-3.93 (m, 1H), 3.89-3.82 (m, 1H), 3.52 (s, 3H), 3.46-3.36 (m, 2H), 3.30 (dt, J=3.6, 8.8 Hz, 1H), 2.62-2.51 (m, 3H), 2.33-2.26 (m, 1H), 2.20-2.12 (m, 1H), 1.97 (s, 2H), 1.38 (s, 3H), 1.04-0.98 (m, 9H).

### Step 2: methyl 4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

To a solution of methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (8.4 g, 11.98 mmol, 1 eq) in DMSO (80 mL) was added CsF (4.00 g, 26.36 mmol, 2.2 eq). The mixture was stirred at 25° C. for 12 h. LCMS showed starting material was consumed and the desired mass was detected. The reaction was filtered and the filtered concentrated under reduced pressure to give a residue. The crude product was purified by reversed-phase HPLC (0.1% FA condition) to give compound methyl 4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (3.1 g, 55.93% yield) as a white solid.

LC-MS [ESI, M+1]:463.7

¹H NMR (400 MHz, CHLOROFORM-d) δ=7.66 (d, J=1.6 Hz, 1H), 7.41-7.29 (m, 4H), 7.27-7.23 (m, 1H), 6.95 (s, 1H), 4.21 (d, J=6.4 Hz, 1H), 4.18-4.04 (m, 2H), 3.96 (d, J=6.4 Hz, 1H), 3.64 (s, 3H), 3.60 (d, J=8.8 Hz, 1H), 3.55-3.45 (m, 2H), 3.39 (dt, J=3.6, 8.8 Hz, 1H), 3.34 (s, 1H), 2.64 (s, 3H), 2.37 (td, J=8.4, 12.0 Hz, 1H), 2.30-2.18 (m, 2H), 2.06-1.99 (m, 1H), 1.46 (s, 3H).

### Step 3: methyl 1-[8-methyl-6-[(3R)-3-methyl-3-phenyl-pyrrolidin-1-yl]-[1,2,4]triazolo[1,5-a]pyridin-2-yl]-4-(ptolylsulfonyloxymethyl)-2-oxabicyclo[2.1.1]hexane-5-carboxylate

To a solution of methyl 4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (3.1 g, 6.70 mmol, 1 eq) in DCM (50 mL) was added TosCl (1.92 g, 10.05 mmol, 1.5 eq), DMAP (81.88 mg, 670.21 μmol, 0.1 eq) and TEA (2.03 g, 20.11 mmol, 2.80 mL, 3 eq). The mixture was stirred at 0-25° C. for 1 h. LCMS showed starting material was consumed and the desired mass was detected. The mixture was concentrated in vacuum and extracted with DCM (3 mL×3). The combined organic layer was washed with brine (3 mL), dried over sodium sulfate, concentrated in vacuum to afford crude. The residue was purified by flash silica gel chromatography (SiO₂, PE:EtOAc=5:1, Rf=0.3) to give compound methyl 1-[8-methyl-6-[(3R)-3-methyl-3-phenyl-pyrrolidin-1-yl]-[1,2,4]triazolo[1,5-a]pyridin-2-yl]-4-(ptolylsulfonyloxymethyl)-2-oxabicyclo[2.1.1]hexane-5-carboxylate (3.3 g, 79.84% yield) as a green solid.

LC-MS [ESI, M+1]:617.3

¹H NMR (400 MHz, CHLOROFORM-d) δ=7.83 (d, J=8.4 Hz, 2H), 7.64 (d, J=1.6 Hz, 1H), 7.43-7.33 (m, 4H), 7.33-7.29 (m, 2H), 7.27-7.23 (m, 1H), 6.94 (d, J=0.8 Hz, 1H), 4.60-4.42 (m, 2H), 4.07 (d, J=6.0 Hz, 1H), 3.89 (d, J=6.0 Hz, 1H), 3.59 (d, J=8.8 Hz, 1H), 3.56 (s, 3H), 3.52-3.44 (m, 2H), 3.38 (dt, J=3.6, 8.8 Hz, 1H), 3.28 (s, 1H), 2.62 (s, 3H), 2.46 (s, 3H), 2.40-2.18 (m, 3H), 1.99 (d, J=8.2 Hz, 1H), 1.46 (s, 3H).

### Step 4: methyl 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate

To a solution of methyl 1-[8-methyl-6-[(3R)-3-methyl-3-phenyl-pyrrolidin-1-yl]-[1,2,4]triazolo[1,5-a]pyridin-2-yl]-4-(p-tolylsulfonyloxymethyl)-2-oxabicyclo[2.1.1]hexane-5-carboxylate (3.3 g, 5.35 mmol, 1 eq) in DMI (35 mL) was added Cs₂CO₃ (5.23 g, 16.05 mmol, 3 eq) and tert-butyl N-tert-butoxycarbonylcarbamate (2.33 g, 10.70 mmol, 2 eq). The mixture was stirred at 75° C. for 2 h. LCMS showed starting material was consumed and the desired mass was detected. The mixture was concentrated in vacuum and extracted with EtOAc (40 mL×3). The combined organic layer was washed with brine (40 mL), dried over sodium sulfate, concentrated in vacuum to afford crude. The crude product was purified by reversed-phase HPLC (0.1% FA condition) to give compound methyl 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (2.9 g, 81.90% yield) as a green solid.

LC-MS [ESI, M+1]: 662.5

¹H NMR (400 MHz, CHLOROFORM-d) δ=7.70-7.61 (m, 1H), 7.39-7.29 (m, 4H), 7.27-7.23 (m, 1H), 6.96-6.91 (m, 1H), 4.32 (d, J=6.0 Hz, 1H), 4.25-4.10 (m, 2H), 3.90 (d, J=6.0 Hz, 1H), 3.62-3.57 (m, 3H), 3.54-3.44 (m, 2H), 3.38 (dt, J=3.6, 8.8 Hz, 1H), 3.23 (br s, 1H), 2.64 (s, 3H), 2.43-2.33 (m, 1H), 2.29-2.17 (m, 2H), 2.01 (s, 2H), 1.54-1.50 (m, 18H), 1.46 (s, 3H).

### Step 5: 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid

To a solution of methyl 4-[[bis(tert-butoxycarbonyl)amino]methyl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexane-5-carboxylate (2.9 g, 4.38 mmol, 1 eq) in THF (20 mL) and Water (10 mL) was added LiOH·H₂O (91.94 mg, 2.19 mmol, 0.5 eq) and NaOH (525.81 mg, 13.15 mmol, 3 eq). The mixture was stirred at 40° C. for 1 h. LCMS showed starting material was consumed and the desired mass was detected. The reaction was filtered and the filtered concentrated under reduced pressure to give a residue. The crude product was purified by reversed-phase HPLC (0.1% FA condition) to give compound 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (2.6 g, 91.60% yield) as a green solid.

LC-MS [ESI, M+1]: 648.3.

¹H NMR (400 MHz, CHLOROFORM-d) δ=7.62 (s, 1H), 7.41-7.34 (m, 2H), 7.33-7.27 (m, 2H), 7.27-7.23 (m, 1H), 7.03 (s, 1H), 4.48-4.20 (m, 2H), 4.19-4.18 (m, 1H), 4.19-4.10 (m, 1H), 3.95-3.86 (m, 1H), 3.60 (br d, J=8.8 Hz, 1H), 3.56-3.45 (m, 2H), 3.43-3.33 (m, 1H), 3.10-2.97 (m, 1H), 2.59 (s, 3H), 2.46-2.34 (m, 1H), 2.32-2.17 (m, 2H), 2.09-1.88 (m, 3H), 1.55-1.45 (m, 18H).

### Step 6: isobutoxycarbonyl 4-[[bis(tert-butoxycarbonyl)amino]methyl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexane-5-carboxylate

To a solution of 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (2 g, 3.09 mmol, 1 eq) in DCM (20 mL) was added TEA (312.43 mg, 3.09 mmol, 429.75 μL, 1 eq) and isobutyl carbonochloridate (548.20 mg, 4.01 mmol, 525.09 μL, 1.3 eq). The mixture was stirred at 0° C. for 0.5 h. TLC showed the starting material was consumed completely. The reaction was filtered and the filtrate was concentrated under reduced pressure to give compound isobutoxycarbonyl 4-[[bis(tert-butoxycarbonyl)amino]methyl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexane-5-carboxylate (2 g, crude) as a green solid.

### Step 7: tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexan-4-yl]methyl]carbamate

To a solution of isobutoxycarbonyl 4-[[bis(tert-butoxycarbonyl)amino]methyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (2 g, 2.67 mmol, 1 eq) in DCM (21 mL) was added TEA (541.21 mg, 5.35 mmol, 744.44 μL, 2 eq) and 2-methylisothiourea (1.93 g, 21.39 mmol, 8 eq) in H₂O (5 mL). The mixture was stirred at 25° C. for 0.1 h. TLC showed the starting material was consumed completely. The mixture was concentrated in vacuum and extracted with DCM (30 mL×3). The combined organic layer was washed with brine (30 mL), dried over sodium sulfate, concentrated in vacuum to afford compound tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexan-4-yl]methyl]carbamate (1.8 g, 93.50% yield) as a green solid.

### Step 8: tert-butyl N-[[5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]-N-tert-butoxycarbonyl-carbamate

To a solution of tert-butyl N-tert-butoxycarbonyl-N-[[1-[8-methyl-6-[(3R)-3-methyl-3-phenyl-pyrrolidin-1-yl]-[1,2,4]triazolo[1,5-a]pyridin-2-yl]-5-[(methylsulfanylcarbonimidoyl)carbamoyl]-2-oxabicyclo[2.1.1]hexan-4-yl]methyl]carbamate (1.8 g, 2.50 mmol, 1 eq) in EtOH (20 mL) was added hydrazine;hydrate (1.47 g, 25.00 mmol, 1.43 mL, 85% purity, 10 eq) and TEA (2.53 g, 25.00 mmol, 3.48 mL, 10 eq). The mixture was stirred at 70° C. for 1 hr. TLC showed the starting material was consumed completely. The mixture was filtered, and concentrated under pressure to give the product. The mixture was concentrated in vacuum and extracted with DCM (30 mL×3). The combined organic layer was washed with brine (30 mL), dried over sodium sulfate, concentrated in vacuum to afford crude. The residue was purified by flash silica gel chromatography (SiO₂, DCM:MeOH=10:1, Rf=0.3) to give compound tert-butyl N-[[5-(3-amino-1H-1,2,4-triazol-5-yl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]-N-tert-butoxycarbonyl-carbamate (660 mg, 38.49% yield) as a green solid.

¹H NMR (400 MHz, CHLOROFORM-d) δ=7.64 (br s, 1H), 7.40-7.33 (m, 2H), 7.33-7.28 (m, 2H), 7.27-7.22 (m, 1H), 6.97 (s, 1H), 4.39-4.21 (m, 2H), 4.12 (q, J=7.2 Hz, 1H), 3.94-3.77 (m, 2H), 3.66-3.56 (m, 1H), 3.55-3.44 (m, 2H), 3.43-3.36 (m, 1H), 2.61 (s, 3H), 2.43-2.20 (m, 4H), 1.61-1.19 (m, 21H).

### Step 9: tert-butyl N-tert-butoxycarbonyl-N-[[5-[2-ethylsulfonyl-5-(ethylsulfonylamino)-1,2,4-triazol-3-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate

To a solution of tert-butyl N-[[5-(3-amino-1H-1,2,4-triazol-5-yl)-1-[8-methyl-6-[(3R)-3-methyl-3-phenyl-pyrrolidin1-yl]-[1,2,4]triazolo[1,5-a]pyridin-2-yl]-2-oxabicyclo[2.1.1]hexan-4-yl]methyl]-N-tert-butoxycarbonyl-carbamate (330 mg, 481.18 μmol, 1 eq) in DCM (3 mL) was added TEA (243.45 mg, 2.41 mmol, 334.87 μL, 5 eq) and ethanesulfonyl chloride (309.35 mg, 2.41 mmol, 227.96 μL, 5 eq). The mixture was stirred at 0° C. for 0.1 h. LCMS showed starting material was consumed and the desired mass was detected. The mixture was concentrated in vacuum and extracted with EtOAc (3 mL×3). The combined organic layer was washed with brine (3 mL), dried over sodium sulfate, concentrated in vacuum to afford compound tert-butyl N-tert-butoxycarbonyl-N-[[5-[2-ethylsulfonyl-5-(ethylsulfonylamino)-1,2,4-triazol-3-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate (400 mg, crude) as a yellow solid.

LC-MS [ESI, M+1]: 870.3

### Step 10: tert-butyl N-[[5-[3-(ethylsulfonylamino)-1H-1,2,4-triazol-5-yl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methyl]carbamate

To a solution of tert-butyl N-tert-butoxycarbonyl-N-[[5-[2-ethylsulfonyl-5-(ethylsulfonylamino)-1,2,4-triazol-3-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate (150 mg, 172.40 μmol, 1 eq) in MeOH (3 mL) was added LiOH·H₂O (21.70 mg, 517.21 μmol, 3 eq). The mixture was stirred at 25° C. for 0.1 h. LCMS showed starting material was consumed and the desired mass was detected. The mixture was concentrated in vacuum and diluted with EtOAc (3 mL). The combined organic layer was washed with brine (3 mL), dried over sodium sulfate, concentrated in vacuum to afford crude. The crude product was purified by reversed-phase HPLC (0.1% FA condition) to give compound tert-butyl N-[[5-[3-(ethylsulfonylamino)-1H-1,2,4-triazol-5-yl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methyl]carbamate (110 mg, 94.13% yield) as a yellow solid.

LC-MS [ESI, M+1]: 678.3

### Step 11: N-[5-[4-(aminomethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]ethanesulfonamide

To a solution of tert-butyl N-[[5-[3-(ethylsulfonylamino)-1H-1,2,4-triazol-5-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]carbamate (100 mg, 147.53 μmol, 1 eq) in DCM (2 mL) and ACN (2 mL) was added TsOH (76.22 mg, 442.60 μmol, 3 eq). The mixture was stirred at 25° C. for 12 h. LCMS showed starting material was consumed and the desired mass was detected. The reaction was filtered and the filtered concentrated under reduced pressure to give a residue. The crude product was purified by reversed-phase HPLC (0.1% FA condition) to give compound N-[5-[4-(aminomethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]ethanesulfonamide (60 mg, 70.40% yield) as a white solid.

LC-MS [ESI, M+1]: 578.4

### Step 12: methyl 4-[[5-[3-(ethylsulfonylamino)-1H-1,2,4-triazol-5-yl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methylamino]-2,2-dimethyl-4-oxobutanoate

To a solution of N-[5-[4-(aminomethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]ethanesulfonamide (20 mg, 34.62 μmol, 1 eq) and 4-methoxy-3,3-dimethyl-4-oxo-butanoic acid (6.65 mg, 41.54 μmol, 1.2 eq) in DMF (1 mL) was added T4P (18.71 mg, 51.93 μmol, 1.5 eq) and DIEA (13.42 mg, 103.86 μmol, 18.09 μL, 3 eq). The mixture was stirred at 25° C. for 1 h. LCMS showed starting material was consumed and the desired mass was detected. The reaction was filtered and purified by reversed-phase HPLC (0.1% FA condition) to give compound methyl 4-[[5-[3-(ethylsulfonylamino)-1H-1,2,4-triazol-5-yl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methylamino]-2,2-dimethyl-4-oxobutanoate (15 mg, 60.19% yield) as a white solid.

LC-MS [ESI, M+1]: 720.6

### Step 13: 4-(((5-(3-(ethylsulfonamido)-1H-1,2,4-triazol-5-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)amino)-2,2-dimethyl-4-oxobutanoic acid

To a solution of methyl 4-[[5-[3-(ethylsulfonylamino)-1H-1,2,4-triazol-5-yl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methylamino]-2,2-dimethyl-4-oxobutanoate (15 mg, 20.84 μmol, 1 eq) in THF (0.5 mL) and Water (0.3 mL) was added NaOH (1 M, 20.84 μL, 1 eq). The mixture was stirred at 0° C. for 0.5 h. LCMS showed starting material was consumed and the desired mass was detected. The reaction was filtered and the filtered concentrated under reduced pressure to give a residue. The residue was purified by prep-HPLC (column: CD25-WePure Biotech XPT PHS C18 150*25*7 um; mobile phase: [H₂O (0.225% FA)—ACN]; gradient:33%-63% B over 10.0 min). The result solution was lyophilized to give a compound 4-(((5-(3-(ethylsulfonamido)-1H-1,2,4-triazol-5-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)amino)-2,2-dimethyl-4-oxobutanoic acid (9.27 mg, 63.03% yield, 100% purity) as a white solid.

LC-MS [ESI, M+1]: 706.5

¹H NMR (400 MHz, METHANOL-d4) δ=7.88 (br s, 1H), 7.42-7.27 (m, 4H), 7.26-7.15 (m, 2H), 3.98 (br d, J=5.2 Hz, 1H), 3.91-3.83 (m, 1H), 3.80-3.64 (m, 2H), 3.59-3.47 (m, 3H), 3.46-3.33 (m, 2H), 3.10 (br d, J=6.0 Hz, 2H), 2.66-2.48 (m, 5H), 2.39-2.24 (m, 3H), 2.20 (br d, J=7.6 Hz, 1H), 1.42 (s, 3H), 1.36-1.22 (m, 9H).

| Compound | | |
|---|---|---|
| Number | Synthesis | LC/MS |
| 547 | Example 78 | m/z 706.5 [M + H]⁺ |
| 548 | Similar to Example 78 | m/z 578.2 [M + H]⁺ |
| 549 | Similar to Example 78 | m/z 606.2 [M + H]⁺ |
| 550 | Similar to Example 78 | m/z 583.2 [M + H]⁺ |
| 551 | Similar to Example 78 | m/z 529.2 [M + H]⁺ |
| 552 | Similar to Example 78 | m/z 584.68 [M + H]⁺ |
| 553 | Similar to Example 78 | m/z 584.68 [M + H]⁺ |
| 554 | Similar to Example 78 | m/z 584.2 [M + H]⁺ |
| 555 | Similar to Example 78 | m/z 597.1 [M + H]⁺ |
| 556 | Similar to Example 78 | m/z 597.2 [M + H]⁺ |
| 557 | Similar to Example 78 | m/z 569.1 [M + H]⁺ |
| 558 | Similar to Example 78 | m/z 606.2 [M + H]⁺ |
| 559 | Similar to Example 78 | m/z 583.4 [M + H]⁺ |
| 560 | Similar to Example 78 | m/z 623.3 [M + H]⁺ |
| 561 | Similar to Example 78 | m/z 646.77 [M + H]⁺ |
| 562 | Similar to Example 78 | m/z 646.77 [M + H]⁺ |
| 563 | Similar to Example 78 | m/z 607.2 [M + H]⁺ |
| 564 | Similar to Example 78 | m/z 621.2 [M + H]⁺ |
| 565 | Similar to Example 78 | m/z 633.4 [M + H]⁺ |
| 566 | Similar to Example 78 | m/z 633.4 [M + H]⁺ |
| 567 | Similar to Example 78 | m/z 637.1 [M + H]⁺ |
| 568 | Similar to Example 78 | m/z 635.4 [M + H]⁺ |

### Example 79

### 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoic acid (Compound 569)

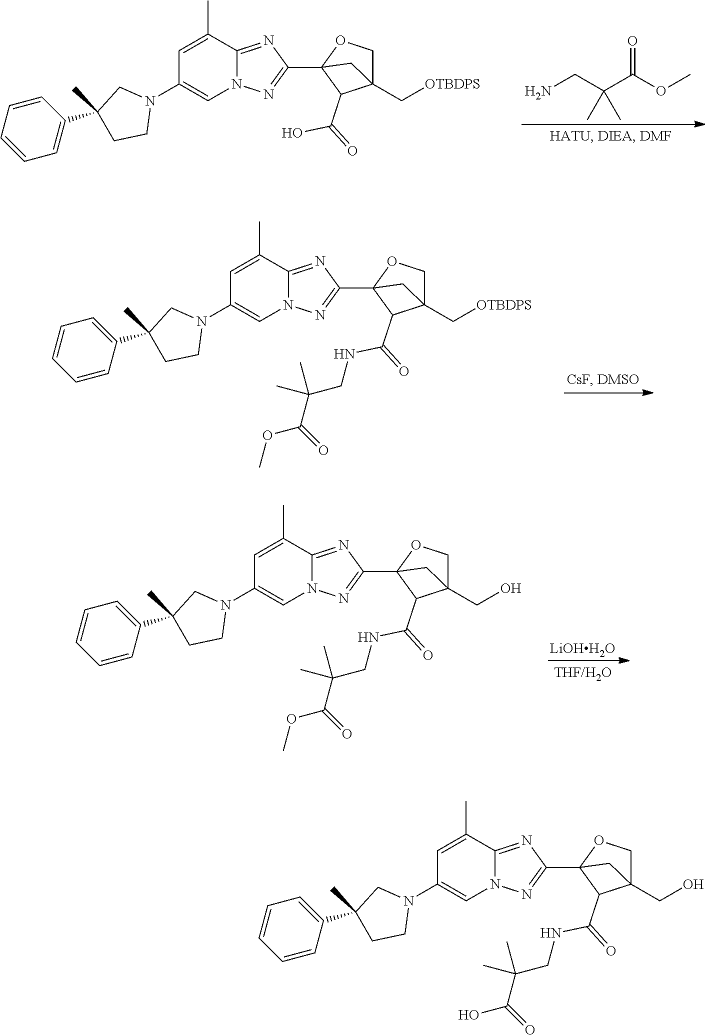

### Step 1: methyl 3-[[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoate

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (35 mg, 50.95 μmol, 1 eq) and methyl 3-amino-2,2-dimethylpropanoate (17.08 mg, 101.91 μmol, 2 eq) in DMF (0.5 mL) was added HATU (29.06 mg, 76.43 μmol, 1.5 eq) and DIEA (19.76 mg, 152.86 μmol, 26.62 μL, 3 eq). The mixture was stirred at 25° C. for 0.5 h. LCMS showed starting material was consumed and one main peak with desired mass was detected. The mixture was quenched with H₂O (10 mL) and extracted with ethyl acetate (10 mL×3), the combined organic phase was dried over anhydrous sodium sulfate, filtered and concentrated to give a residue. The residue was purified by column chromatography (SiO₂, hexanes:EtOAc=0:1, Rf=0.32) to give compound methyl 3-[[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoate (35 mg, 86% yield) as yellow solid.

LC-MS [ESI, M+1]: 800.8.

### Step 2: methyl 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoate

To a solution of methyl 3-[[4-[[tert-butyl(diphenyl)silyl]oxymethyl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexane-5-carbonyl]amino]-2,2-dimethylpropanoate (30 mg, 37.50 μmol, 1 eq) in DMSO (1 mL) was added CsF (11.39 mg, 74.99 μmol, 2 eq). The mixture was stirred at 25° C. for 12 h. LCMS showed starting material was consumed and one main peak with desired mass was detected. The mixture was quenched with H₂O (3 mL) and extracted with ethyl acetate (10 mL×3), the combined organic phase was dried over anhydrous sodium sulfate, filtered and concentrated to give compound methyl 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoate (18 mg, 85% yield) as yellow solid.

LC-MS [ESI, M+1]: 562.3.

### Step 3: 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoic acid

To a solution of methyl 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoate (18 mg, 32.05 μmol, 1 eq) in THF (1 mL) was added TMSOK (8.22 mg, 64.09 μmol, 2 eq) and H₂O (0.3 mL). The mixture was stirred at 25° C. for 1 h. LCMS showed starting material was consumed and the desired mass was detected. The reaction was concentrated under reduced pressure to give a residue. The residue was purified by prep-HPLC purification (mobile phase: [water(FA)—ACN]; B %: 28%-58%, 10 min), the mixture was concentrated and lyophilized to give compound 3-[[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl]amino]-2,2-dimethyl-propanoic acid (15.35 mg, 86% yield) as a white solid.

LC-MS [ESI, M+1]: 548.4.

¹H NMR (400 MHz, CHLOROFORM-d) δ=9.19 (br s, 1H), 7.61 (br s, 1H), 7.35 (br d, J=7.2 Hz, 2H), 7.33-7.29 (m, 2H), 7.26 (br s, 1H), 6.96 (br s, 1H), 4.73 (s, 1H), 4.11 (br d, J=12.8 Hz, 2H), 3.98 (br d, J=11.2 Hz, 1H), 3.90-3.82 (m, 1H), 3.58 (br d, J=8.8 Hz, 2H), 3.48 (br dd, J=8.0, 14.8 Hz, 3H), 3.41-3.35 (m, 1H), 3.00 (s, 1H), 2.57 (br s, 3H), 2.43-2.33 (m, 1H), 2.31-2.21 (m, 1H), 2.20-2.10 (m, 1H), 1.97 (br dd, J=3.2, 7.2 Hz, 1H), 1.46 (s, 3H), 1.26 (br d, J=6.8 Hz, 6H).

### Example 80

### (5-(2-methyl-2 h-tetrazol-5-yl)-1-(8-methyl-6-((r)-3-methyl-3-phenylpyrrolidin-1-yl)-[1,2,4]triazolo[1,5-a]pyridin-2-yl)-2-oxabicyclo[2.1.1]hexan-4-yl)methanol (Compound 570)

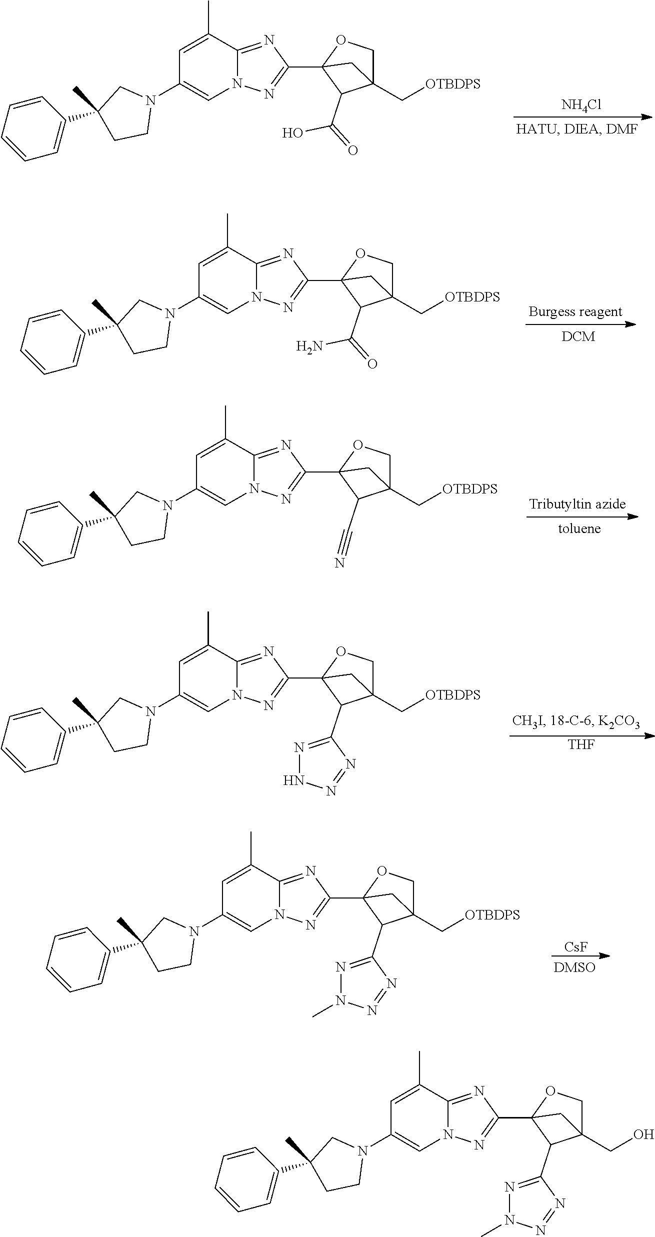

### Step 1: 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxamide

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.8 g, 2.62 mmol, 1 eq) and NH₄Cl (1.40 g, 26.20 mmol, 10 eq) in DMF (20 mL) was added HATU (2.99 g, 7.86 mmol, 3 eq) and DIEA (1.69 g, 13.10 mmol, 2.28 mL, 5 eq). The mixture was stirred at 25° C. for 1 h. LCMS showed starting material was consumed and the desired mass was detected. The mixture was diluted with H₂O (100 mL) and extracted with EtOAc (120 mL×5) and washed with brine (100 mL×4) the organic phase was dried, filtered, and concentrated under pressure to give the crude product. The crude product was purified by column chromatography (SiO₂, DCM:MeOH=10:1, Rf=0.5) to give compound 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxamide (1.84 g, 95% yield) as a green solid.

LC-MS [ESI, M+1]: 686.2

¹H NMR (400 MHz, CHLOROFORM-d) δ=8.51 (s, 1H), 8.02 (s, 1H), 7.76-7.61 (m, 5H), 7.48-7.29 (m, 10H), 7.27-7.23 (m, 1H), 7.01-6.92 (m, 1H), 4.28 (d, J=11.2 Hz, 1H), 4.10 (s, 1H), 4.05-3.92 (m, 2H), 3.60 (d, J=8.0 Hz, 1H), 3.53-3.44 (m, 2H), 3.44-3.33 (m, 1H), 3.06 (s, 1H), 2.65-2.57 (m, 3H), 2.43-2.34 (m, 1H), 2.30-2.24 (m, 1H), 2.23-2.13 (m, 2H), 1.47 (s, 3H), 1.08 (s, 9H)

### Step 2: 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carbonitrile

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (300 mg, 437.36 μmol, 1 eq) in DCM (3 mL) was added methoxycarbonyl-(triethylammonio)sulfonyl-azanide (208.45 mg, 874.73 μmol, 2 eq) and 4A MS (100 mg). The mixture was stirred at 25° C. for 12 h. LCMS showed starting material was consumed and the desired mass was detected. The mixture was diluted with H₂O (5 mL) and extracted with EtOAc (10 mL×3), the organic phase was dried, filtered, and concentrated under pressure to give the crude product. The crude product was purified by column chromatography (SiO₂, PE:EtOAc=2:1, Rf=0.6) to give compound 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carbonitrile (270 mg, 92% yield) as a green solid.

LC-MS [ESI, M+1]: 668.3

¹H NMR (400 MHz, CHLOROFORM-d) δ=7.75-7.57 (m, 5H), 7.51-7.40 (m, 5H), 7.40-7.34 (m, 2H), 7.34-7.28 (m, 2H), 7.27-7.22 (m, 2H), 6.96 (s, 1H), 4.22 (d, J=6.8 Hz, 1H), 4.04-3.94 (m, 3H), 3.65-3.56 (m, 1H), 3.56-3.44 (m, 2H), 3.39 (dt, J=3.6, 8.4 Hz, 1H), 3.26 (s, 1H), 2.67-2.58 (m, 3H), 2.43-2.31 (m, 2H), 2.30-2.21 (m, 1H), 2.09 (d, J=8.0 Hz, 1H), 1.62 (s, 3H), 1.10 (s, 9H)

### Step 3: 2-(4-(((tert-butyldiphenylsilyl)oxy)methyl)-5-(2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonitrile (220 mg, 329.38 μmol, 1 eq) in Tol. (2 mL) was added TMSN₃ (379.48 mg, 3.29 mmol, 433.19 μL, 10 eq) and [acetoxy(dibutyl)stannyl]acetate (1.16 g, 3.29 mmol, 883.95 μL, 10 eq). The mixture was stirred at 120° C. for 2 h. LCMS showed starting material was consumed and the desired mass was detected. The mixture was quenched with H₂O (2 mL) and extracted with EtOAc (5 mL×3), the organic phase was dried, filtered, and concentrated under pressure to give the crude product. The residue was purified by column chromatography (SiO₂, DCM:MeOH=10:1, Rf=0.4) to give compound 2-(4-(((tert-butyldiphenylsilyl)oxy)methyl)-5-(2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine (200 mg, 85% yield) as a green gum.

LC-MS [ESI, M+1]: 711.3.

### Step 4: 2-(4-(((tert-butyldiphenylsilyl)oxy)methyl)-5-(2-methyl-2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of tert-butyl-[[1-[8-methyl-6-[(3R)-3-methyl-3-phenyl-pyrrolidin-1-yl]-[1,2,4]triazolo[1,5-a]pyridin-2-yl]-5-(2H-tetrazol-5-yl)-2-oxabicyclo[2.1.1]hexan-4-yl]methoxy]-diphenyl-silane (200 mg, 281.32 μmol, 1 eq) in THF (3 mL) was added K₂CO₃ (77.76 mg, 562.64 μmol, 2 eq) 18-C-6 (7.44 mg, 28.13 μmol, 0.1 eq) and CH₃I (199.65 mg, 1.41 mmol, 87.57 μL, 5 eq). The mixture was stirred at 40° C. for 1 h. LCMS showed starting material was consumed and one main peak with desired mass was detected. The mixture was diluted with H₂O (100 mL) and extracted with EtOAc (120 mL×5) and washed with brine (100 mL×4) the organic phase was dried, filtered, and concentrated under pressure to give the crude product. The crude product. was purified by reversed-phase HPLC (0.1% FA condition) and lyophilizated to. give compound 2-(4-(((tert-butyldiphenylsilyl)oxy)methyl)-5-(2-methyl-2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine (100 mg, 49% yield) as a green solid.

LC-MS [ESI, M+1]: 725.4

1H NMR (400 MHz, CHLOROFORM-d) δ=7.67-7.54 (m, 5H), 7.46-7.31 (m, 11H), 7.00-6.90 (m, 1H), 4.30-4.21 (m, 2H), 4.16-4.11 (m, 3H), 3.99 (d, J=6.0 Hz, 1H), 3.81 (s, 1H), 3.68-3.54 (m, 2H), 3.52-3.43 (m, 2H), 3.42-3.35 (m, 1H), 2.64-2.57 (m, 3H), 2.44-2.23 (m, 3H), 2.22-2.16 (m, 1H), 1.46 (d, J=5.2 Hz, 3H), 1.09-1.00 (m, 9H)

### Step 5: (5-(2-methyl-2H-tetrazol-5-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol

To a solution of tert-butyl-[[1-[8-methyl-6-[(3R)-3-methyl-3-phenyl-pyrrolidin-1-yl]-[1,2,4]triazolo[1,5-a]pyridin-2-yl]-5-(2-methyltetrazol-5-yl)-2-oxabicyclo[2.1.1]hexan-4-yl]methoxy]-diphenyl-silane (90 mg, 124.14 μmol, 1 eq) in DMSO (0.5 mL) was added CsF (37.72 mg, 248.29 μmol, 2 eq). The mixture was stirred at 25° C. for 0.5 h. LCMS showed starting material was consumed and one main peak with desired mass was detected. The mixture was filtered, and concentrated under pressure to give a residue. The residue by prep-HPLC (column: Phenomenex luna C18 150*25 mm*10 um; mobile phase: [water(FA)—ACN]; gradient:38%-68% B over 10 min and column: Phenomenex luna C18 150*25 mm*10 um; mobile phase: [water(FA)—ACN]; gradient:32%-62% B over 10 min) and lyophilizated to give compound (5-(2-methyl-2H-tetrazol-5-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol (5.82 mg, 10% yield) as a yellow solid.

LC-MS [ESI, M+1]: 487.2

1H NMR (400 MHz, CHLOROFORM-d) δ=7.66 (d, J=1.6 Hz, 1H), 7.41-7.34 (m, 2H), 7.33-7.29 (m, 2H), 7.27-7.23 (m, 1H), 6.94 (s, 1H), 4.32-4.23 (m, 4H), 4.13 (dd, J=7.2, 12.0 Hz, 1H), 4.08 (d, J=6.4 Hz, 1H), 3.99 (d, J=6.4 Hz, 1H), 3.80 (s, 1H), 3.59 (d, J=8.8 Hz, 1H), 3.55-3.42 (m, 3H), 3.38 (t, J=3.6, 8.8 Hz, 1H), 2.64 (s, 3H), 2.46 (d, J=7.2 Hz, 1H), 2.37 (td, J=8.0, 12.0 Hz, 1H), 2.30-2.19 (m, 2H), 1.46 (s, 3H)

| Compound | | |
|---|---|---|
| Number | Synthesis | LC/MS |
| 571 | Similar to Example 80 | m/z 487.2 [M + H]⁺ |

### Example 81

### N-[5-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]propanamide (Compound 572)

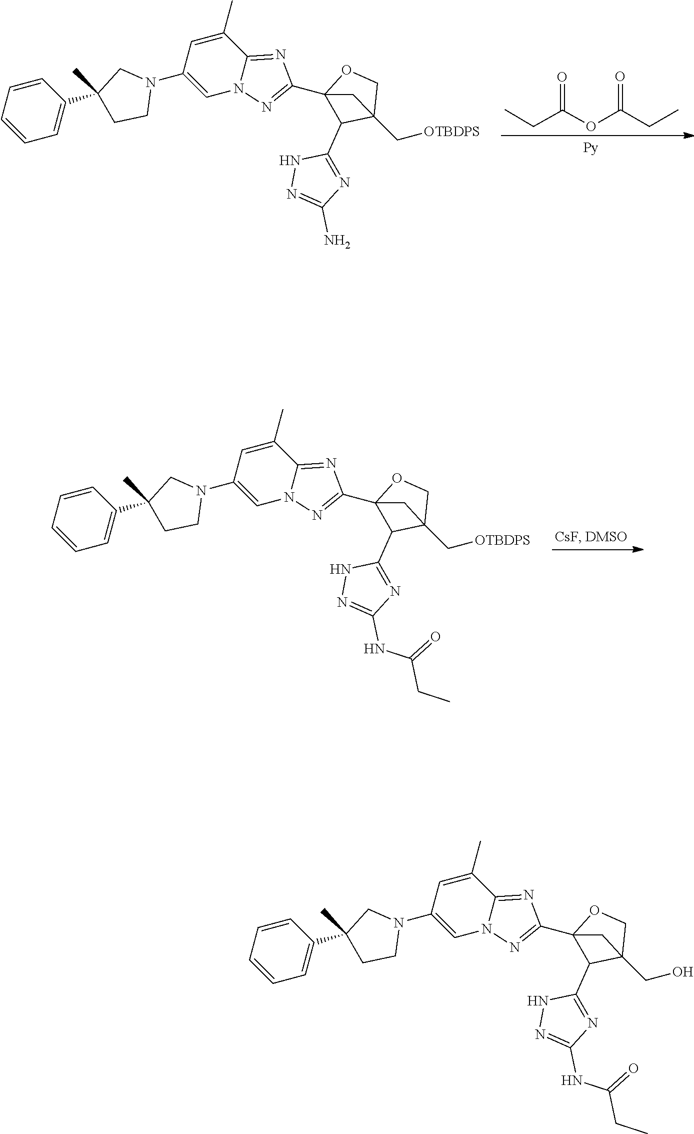

### Step 1: N-[3-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-5-yl]propanamide

To a solution of 5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-amine (40 mg, 55.17 μmol, 1 eq) in Py (1 mL) was added propanoyl propanoate (71.81 mg, 551.75 μmol, 71.09 μL, 10 eq). The mixture was stirred at 50° C. for 1 h. LCMS showed starting material was consumed and one main peak with desired mass was detected. The reaction mixture was quenched by H₂O (3 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.58) to give compound N-[3-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-5-yl]propanamide (40 mg, 93% yield) as yellow solid.

LC-MS [ESI, M+1]: 781.4.

### Step 2: N-[5-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]propanamide

To a solution of N-[5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]propanamide (20 mg, 25.61 μmol, 1 eq) in DMSO (0.5 mL) was added CsF (5.83 mg, 38.41 μmol, 1.5 eq). The mixture was stirred at 25° C. for 12 h. LCMS showed starting material was consumed and the desired mass was detected. The reaction was concentrated under reduced pressure to give a residue. The residue was purified by prep-HPLC purification (mobile phase: [water(FA)—ACN]; B %: 30%-60%, 10 min), the mixture was concentrated and lyophilized to give compound N-[5-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-yl]propanamide (5.06 mg, 35% yield) as a yellow solid.

LC-MS [ESI, M+1]: 543.4.

¹H NMR (400 MHz, CHLOROFORM-d) δ=7.73 (br d, J=3.2 Hz, 1H), 7.41-7.28 (m, 4H), 7.27-7.20 (m, 1H), 6.99 (br s, 1H), 4.24 (br d, J=11.2 Hz, 1H), 4.08 (br d, J=11.2 Hz, 2H), 3.90 (br d, J=6.4 Hz, 1H), 3.61 (br d, J=8.8 Hz, 1H), 3.57-3.47 (m, 2H), 3.46-3.32 (m, 2H), 2.76-2.63 (m, 3H), 2.55 (br d, J=2.4 Hz, 2H), 2.44-2.34 (m, 2H), 2.28 (br dd, J=3.6, 7.2 Hz, 2H), 1.47 (s, 3H), 1.26 (br t, J=7.2 Hz, 3H).

### Example 82

### N-[3-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-5-yl]ethanesulfonamide (Compound 573)

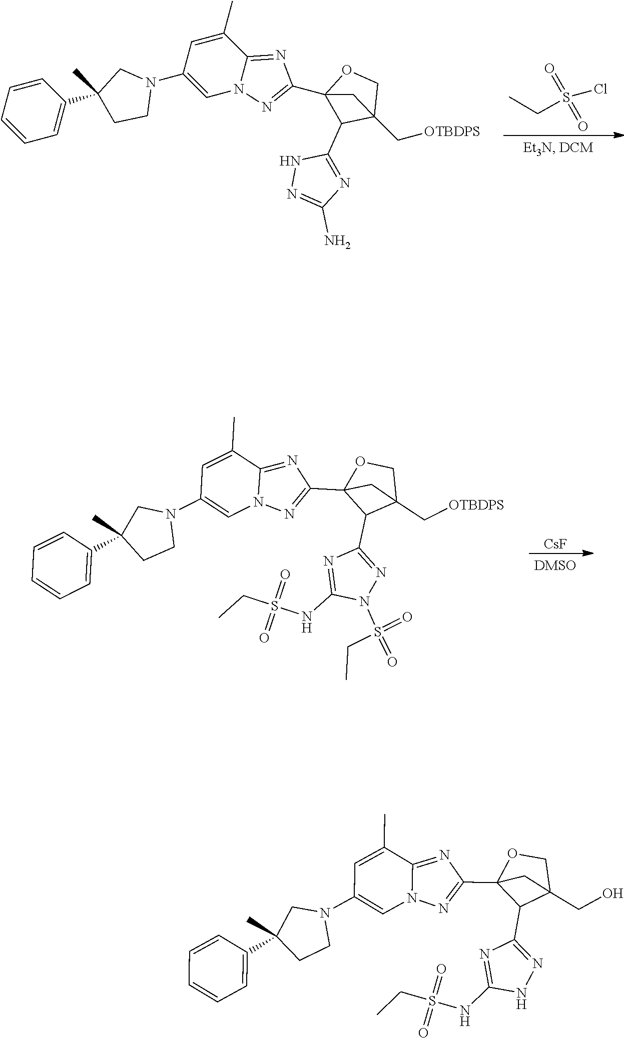

### Step 1: N-[5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-ethylsulfonyl-1,2,4-triazol-3-yl]ethanesulfonamide

To a solution of 5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-3-amine (50 mg, 68.97 μmol, 1 eq) in DCM (2 mL) was added ethanesulfonyl chloride (177.36 mg, 1.38 mmol, 130.70 μL, 20 eq) and Et₃N (20.94 mg, 206.91 μmol, 28.80 μL, 3 eq). The mixture was stirred at 25° C. for 0.5 h. LCMS showed starting material was consumed and one main peak with desired mass was detected. The reaction mixture was quenched by H₂O (3 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, hexanes:EtOAc=0:1, Rf=0.42) to give compound N-[5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-ethylsulfonyl-1,2,4-triazol-3-yl]ethanesulfonamide (30 mg, 48% yield) as yellow solid.

LC-MS [ESI, M+1]: 909.3.

### Step 2: N-[3-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-5-yl]ethanesulfonamide

To a solution of N-[5-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-2-ethylsulfonyl-1,2,4-triazol-3-yl]ethanesulfonamide (25 mg, 27.50 μmol, 1 eq) in DMSO (1 mL) was added CsF (6.27 mg, 41.25 μmol, 1.5 eq). The mixture was stirred at 25° C. for 12 h. LCMS showed starting material was consumed and the desired mass was detected. The reaction was concentrated under reduced pressure to give a residue. The residue was purified by prep-HPLC purification (mobile phase: [water(FA)—ACN]; B %: 31%-61%, 10 min), the mixture was concentrated and lyophilized to give compound N-[3-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-1H-1,2,4-triazol-5-yl]ethanesulfonamide (2.05 mg, 12% yield) as a yellow solid.

LC-MS [ESI, M+1]: 579.1.

¹H NMR (400 MHz, CHLOROFORM-d) δ=7.73 (br s, 1H), 7.42-7.28 (m, 5H), 7.27-7.22 (m, 1H), 7.02 (s, 1H), 4.25-4.15 (m, 1H), 4.15-4.07 (m, 1H), 4.07-3.95 (m, 1H), 3.92 (br d, J=6.4 Hz, 1H), 3.62 (br d, J=8.8 Hz, 1H), 3.60-3.49 (m, 2H), 3.49-3.34 (m, 2H), 3.32 (br s, 1H), 3.26-3.15 (m, 1H), 2.66 (s, 3H), 2.42-2.26 (m, 4H), 1.47 (s, 3H), 1.41 (br t, J=7.2 Hz, 3H).

| Compound | | |
|---|---|---|
| Number | Synthesis | LC/MS |
| 574 | Similar to Example 82 | m/z 651.4 [M + H]⁺ |

### Example 83

### 4-(hydroxymethyl)-1-(8-methyl-6-((r)-3-methyl-3-phenylpyrrolidin-1-yl)-[1,2,4]triazolo[1,5-a]pyridin-2-yl)-2-oxabicyclo[2.1.1]hexane-5-carboxylic acid (Compound 575)

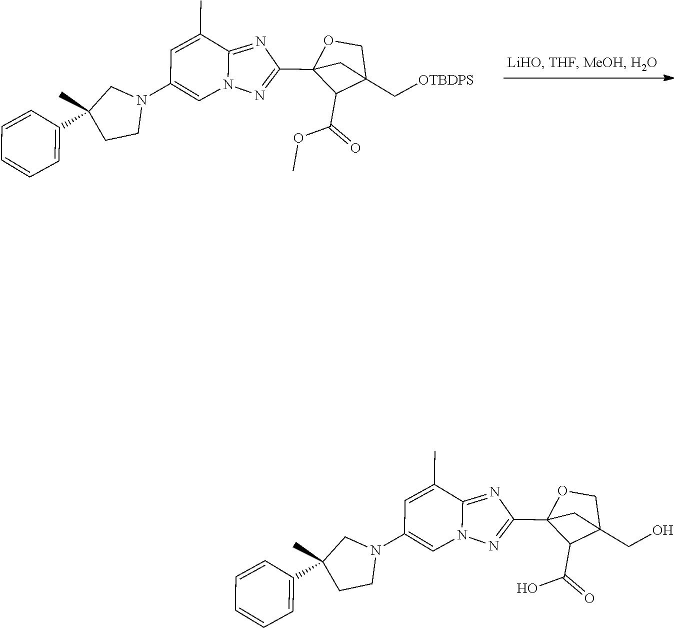

### Step 1: 2-(4-(((tert-butyldiphenylsilyl)oxy)methyl)-5-(2-methyl-2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (70 mg, 99.87 μmol, 1 eq) in THF (0.5 mL) and H2O (0.25 mL) was added LiOH·H₂O (16.76 mg, 399.46 μmol, 4 eq) and MeOH (0.25 mL). The mixture was stirred at 60° C. for 1 h. LCMS showed starting material was consumed and one main peak with desired mass was detected. The mixture was filtered, and concentrated under pressure to give a residue. The residue by prep-HPLC ((column: Phenomenex luna C18 150*25 mm*10 um; mobile phase: [water(FA)—ACN]; gradient:33%-63% B over 10 min) and lyophilizated to give compound 2-(4-(((tert-butyldiphenylsilyl)oxy)methyl)-5-(2-methyl-2H-tetrazol-5-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine (36.8 mg, 81% yield) as a gray solid.

LC-MS [ESI, M+1]: 449.1

1H NMR (400 MHz, CHLOROFORM-d) δ=7.62 (s, 1H), 7.41-7.33 (m, 2H), 7.33-7.28 (m, 2H), 7.27-7.23 (m, 1H), 7.03 (s, 1H), 4.27 (d, J=6.4 Hz, 1H), 4.20-4.12 (m, 1H), 4.09-4.00 (m, 1H), 3.93 (d, J=6.4 Hz, 1H), 3.60 (d, J=8.8 Hz, 1H), 3.55-3.44 (m, 2H), 3.39 (t, J=7.2 Hz, 1H), 3.09 (s, 1H), 2.58 (s, 3H), 2.39 (td, J=8.4, 12.4 Hz, 1H), 2.31-2.23 (m, 1H), 2.20 (d, J=7.6 Hz, 1H), 2.02 (d, J=7.6 Hz, 1H), 1.47 (s, 3H).

### Example 84

### 6-(4-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-4,6-diazaspiro[2.4]heptan-5-one (Compound 576)

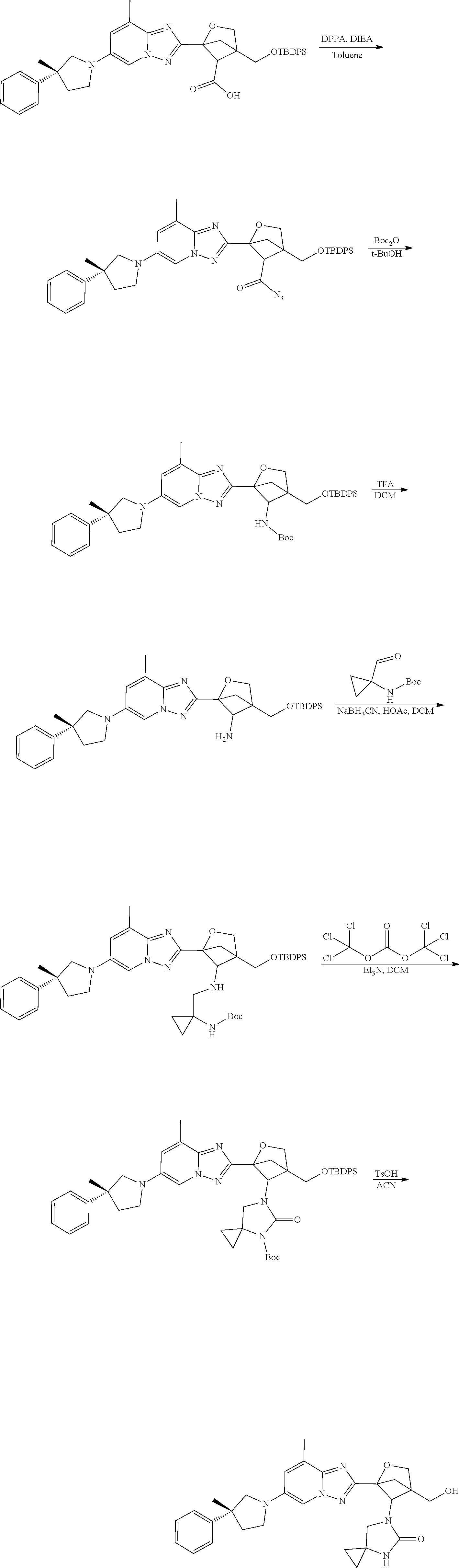

### Step 1: 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl azide

To a solution of 4-[[tert-butyl(diphenyl)silyl]oxymethyl]-1-[8-methyl-6-[(3R)-3-methyl-3-phenyl-pyrrolidin-1l-yl]-[1,2,4]triazolo[1, 5-a]pyridin-2-yl]-2-oxabicyclo[2.1.1]hexane-5-carboxylic acid (500 mg, 727.90 μmol, 1 eq) in toluene (5 mL) was added DIEA (282.22 mg, 2.18 mmol, 380.36 μL, 3 eq), DPPA (100.16 mg, 363.95 μmol, 78.56 μL, 1.5 eq). The mixture was stirred at 25° C. for 12 h. LCMS showed starting material was consumed and one main peak with desired mass was detected. The mixture was quenched with water (3 mL) and extracted with ethyl acetate (5 mL×3), the combined organic phase was dried over anhydrous sodium sulfate, filtered and concentrated to give 4-[[tert-butyl(diphenyl)silyl]oxymethyl]-1-[8-methyl-6-[(3R)-3-methyl-3-phenyl-pyrrolidin-1l-yl]-[1,2,4]triazolo[1,5-a]pyridin-2-yl]-2-oxabicyclo[2.1.1]hexane-5-carbonyl azide (500 mg, crude) as yellow oil.

LC-MS [ESI, M+1]. 712.4.

### Step 2: tert-butyl N-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate

A solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carbonyl azide (500 mg, 702.32 μmol, 1 eq), Boc₂O (766.39 mg, 3.51 mmol, 806.73 μL, 5 eq), t-BuOH (5 mL) was stirred at 100° C. for 3 h. LCMS showed starting material was consumed and one main peak with desired mass was detected. The reaction concentrated under reduced pressure to give a residue. The residue was purified by flash silica gel chromatography (SiO₂, PE:EtOAc=2:1, Rf=0.35) to give compound tert-butyl N-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (280 mg, 369.38 μmol, 52.59% yield) as yellow oil.

LC-MS [ESI, M+1]: 758.4.

¹H NMR (400 MHz, CHLOROFORM-d) δ=7.74-7.60 (m, 5H), 7.45-7.30 (m, 10H), 7.25 (s, 1H), 6.93 (s, 1H), 4.13-3.94 (m, 4H), 3.74 (d, J=6.4 Hz, 1H), 3.58 (br d, J=8.8 Hz, 1H), 3.54-3.43 (m, 2H), 3.42-3.31 (m, 1H), 2.62 (s, 3H), 2.36 (td, J=8.2, 12.0 Hz, 1H), 2.26 (br dd, J=4.0, 7.2 Hz, 1H), 1.99 (br d, J=14.4 Hz, 2H), 1.46 (s, 3H), 1.43-1.28 (m, 9H), 1.08 (s, 9H).

### Step 3: 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine

A solution of tert-butyl N-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]carbamate (280 mg, 369.38 μmol, 1 eq) in TFA (0.3 mL), DCM (3 mL) was stirred at 25° C. for 1 h. LCMS showed starting material was consumed and one main peak with desired mass was detected. The mixture was added water 3 ml, then NaHCO₃ was used to adjust pH=8, and extracted with dichloromethane (3 mL×3), the combined organic phase was dried over anhydrous sodium sulfate, filtered and concentrated to give 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (220 mg, 244.10 μmol, 66.09% yield, 73% purity) as green oil.

LC-MS [ESI, M+1]: 658.2.

### Step 4: tert-butyl N-[1-[[[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]cyclopropyl]carbamate

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-amine (70 mg, 106.40 μmol, 1 eq), tert-butyl N-(1-formylcyclopropyl)carbamate (29.56 mg, 159.59 μmol, 1.5 eq) in MeOH (2 mL) was added AcOH (12.78 mg, 212.79 μmol, 12.18 μL, 2 eq), NaBH₃CN (10.03 mg, 159.59 μmol, 1.5 eq) at 0° C. The mixture was stirred at 25° C. for 1 h. LCMS showed starting material was consumed and the desired mass was detected. The mixture was quenched with water (5 mL) and extracted with dichloromethane (5 mL×3), the combined organic phase was dried over anhydrous sodium sulfate, filtered and concentrated to give a residue. The residue was purified by prep-TLC (SiO₂, PE:EtOAc=2:1, Rf=0.43) to give tert-butyl N-[1-[[[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]cyclopropyl]carbamate (70 mg, 77.44 μmol, 72.7% yield, 91.5% purity) as green oil.

LC-MS [ESI, M+1]: 827.6.

### Step 5: tert-butyl 6-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-5-oxo-4,6-diazaspiro[2.4]heptane-4-carboxylate

To a solution of bis(trichloromethyl) carbonate (86.10 mg, 290.16 μmol, 3 eq) in DCM (1 mL) at 0° C. was added tert-butyl N-[1-[[[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]amino]methyl]cyclopropyl]carbamate (80 mg, 96.72 μmol, 1 eq) and Et₃N (29.36 mg, 290.16 μmol, 40.39 μL, 3 eq) in DCM (1 mL). The mixture was stirred at 25° C. for 1 h. LCMS showed starting material was consumed and one main peak with desired mass was detected. The reaction mixture was quenched with water (5 mL) and extracted with dichloromethane (5 mL×3), the combined organic phase was dried over anhydrous sodium sulfate, filtered and concentrated to give a residue. The residue was purified by flash silica gel chromatography (SiO₂, PE:EtOAc=2:1, Rf=0.49) to give tert-butyl 6-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-5-oxo-4,6-diazaspiro[2.4]heptane-4-carboxylate (26 mg, 30.48 μmol, 31.5% yield) as yellow oil.

LC-MS [ESI, M+1]: 853.3.

### Step 6: 6-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-4,6-diazaspiro[2.4]heptan-5-one

To a solution of tert-butyl 6-[4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-5-oxo-4,6-diazaspiro[2.4]heptane-4-carboxylate (25 mg, 29.30 μmol, 1 eq) in DCM (0.5 mL), ACN (0.5 mL) was added 4-methylbenzenesulfonic acid;hydrate (16.72 mg, 87.91 μmol, 3 eq). The mixture was stirred at 25° C. for 12 h. LCMS showed starting material was consumed and one peak with 31% desired mass was detected. The residue was purified by prep-HPLC (column: CD25-XPT PHS C18 150*25*10 um; mobile phase: [water(FA)—ACN]; gradient:37%-67% B over 10 min) to give 6-[4-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]-4,6-diazaspiro[2.4]heptan-5-one (2.48 mg, 4.66 μmol, 15.9% yield, 96.7% purity) as white solid.

LC-MS [ESI, M+1]: 515.4.

¹H NMR (400 MHz, CHLOROFORM-d) δ=7.63 (s, 1H), 7.42-7.29 (m, 4H), 7.24 (br s, 1H), 6.95 (s, 1H), 4.78 (br d, J=1.6 Hz, 1H), 4.30 (s, 1H), 4.07 (d, J=6.4 Hz, 1H), 4.04-3.95 (m, 2H), 3.93-3.84 (m, 2H), 3.71 (d, J=9.2 Hz, 1H), 3.63-3.44 (m, 4H), 3.39 (dt, J=3.2, 8.6 Hz, 1H), 2.62 (s, 3H), 2.43-2.33 (m, 1H), 2.30-2.20 (m, 1H), 2.11-2.03 (m, 1H), 2.02-1.94 (m, 1H), 1.46 (s, 3H), 0.82-0.65 (m, 3H), 0.58-0.47 (m, 1H).

| Compound | | |
|---|---|---|
| Number | Synthesis | LC/MS |
| 577 | Similar to Example 84 | m/z 531.4 [M + H]⁺ |

### Example 85

### [1-[8-methyl-6-(7-phenyl-2-azaspiro[3.3]heptan-2-yl)-[1,2,4]triazolo[1,5-a]pyridin-2-yl]-5-(3-methyl-1H1,2,4-triazol-5-yl)-2-oxabicyclo[2.1.1]hexan-4-yl]methanol (Compound 578)

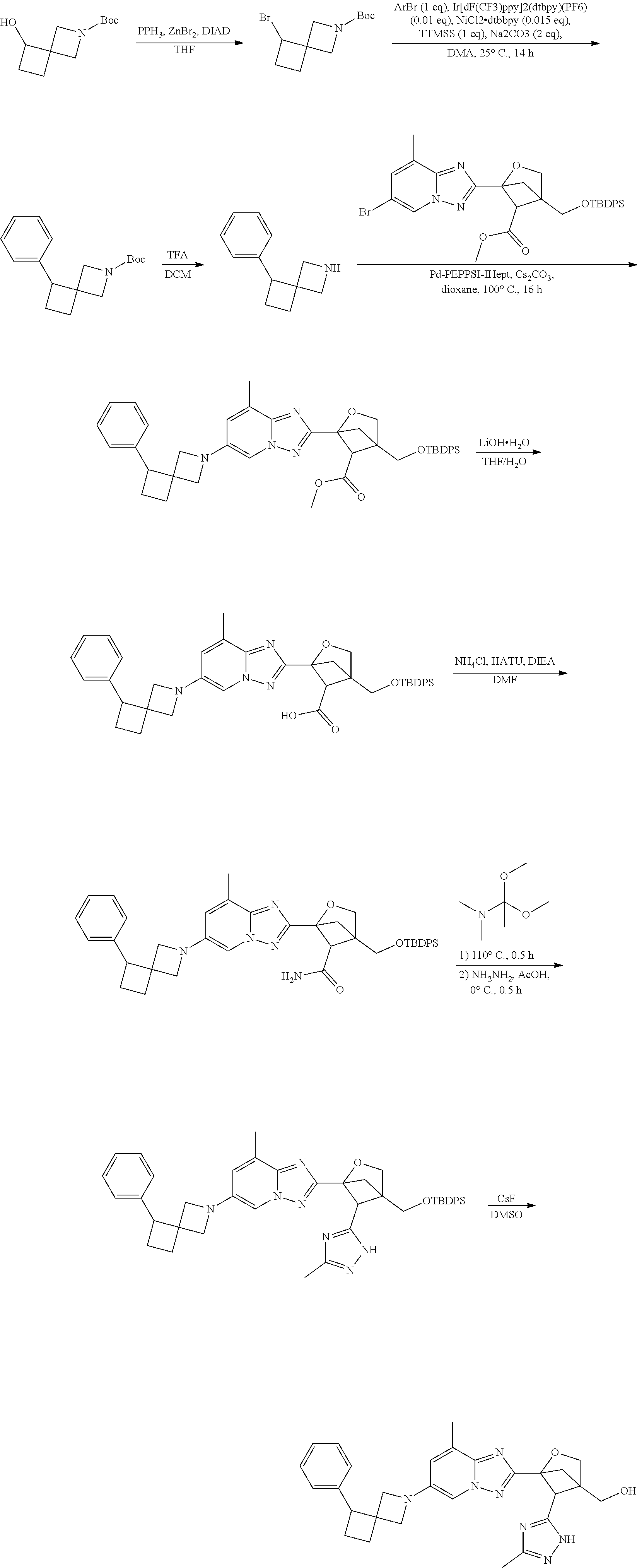

### Step 1: tert-butyl 7-bromo-2-azaspiro[3.3]heptane-2-carboxylate

The tert-butyl 7-hydroxy-2-azaspiro[3.3]heptane-2-carboxylate (1.45 g, 6.80 mmol, 1 eq) in THF (40 mL), PPh₃ (6.60 g, 25.16 mmol, 3.7 eq) was added, followed by a solution of ZnBr₂ (1.99 g, 8.84 mmol, 442.31 μL, 1.3 eq) in THF (12 mL). DIAD (5.09 g, 25.16 mmol, 4.88 mL, 3.7 eq) dissolved in THF (12 mL) was added to the reaction mixture. The mixture was stirred at 25° C. for 12 h. LCMS showed starting material was consumed and the desired mass was detected. The reaction was filtered and the filtered concentrated under reduced pressure to give a residue. The residue was purified by flash silica gel chromatography (SiO₂, PE:EtOAc=5:1, Rf=0.3) to give compound tert-butyl 7-bromo-2-azaspiro[3.3]heptane-2-carboxylate (1 g, 53.26% yield) as a colorless oil.

LC-MS [ESI, M+1]:279.2

¹H NMR (400 MHz, CHLOROFORM-d) δ=4.49-4.35 (m, 2H), 3.89-3.76 (m, 3H), 2.56-2.31 (m, 2H), 2.21-2.02 (m, 2H), 1.48-1.41 (m, 9H).

### Step 2: tert-butyl 7-phenyl-2-azaspiro[3.3]heptane-2-carboxylate

To an 15 mL vial equipped with a stir bar was added tert-butyl 7-bromo-2-azaspiro[3.3]heptane-2-carboxylate (989.41 mg, 3.58 mmol, 1.25 eq),bromobenzene (450 mg, 2.87 mmol, 301.81 μL, 1 eq),Ir[dF(CF3)ppy]2(dtbpy)(PF6) (32.16 mg, 28.66 μmol, 0.01 eq), NiCl₂·dtbbpy (20 mg, 42.99 μmol, 0.015 eq), TTMSS (712.69 mg, 2.87 mmol, 884.23 μL, 1 eq), NaCO₃ (475.77 mg, 5.73 mmol, 2 eq) in DME (10 mL). The vial was sealed and placed under nitrogen was added. The reaction was stirred with a 10 W blue LED lamp (3 cm away), with colling water to keep the reaction temperature at 25° C. for 14 h. LCMS showed starting material was consumed and the desired mass was detected. The reaction was filtered and the filtered concentrated under reduced pressure to give a residue. The crude product was purified by reversed-phase HPLC (0.1% FA condition) to give compound tert-butyl 7-phenyl-2-azaspiro[3.3]heptane-2-carboxylate (350 mg, 44.67% yield) as a yellow oil.

LC-MS [ESI, M-56]:218.7

¹H NMR (400 MHz, CHLOROFORM-d) δ=7.39-7.32 (m, 2H), 7.27-7.22 (m, 1H), 7.17 (d, J=7.6 Hz, 2H), 4.05-3.94 (m, 1H), 3.93-3.87 (m, 1H), 3.66-3.58 (m, 2H), 3.52 (br t, J=8.0 Hz, 1H), 2.22-2.11 (m, 4H), 1.38 (s, 9H).

### Step 3: 7-phenyl-2-azaspiro[3.3]heptane

To a solution of tert-butyl 7-phenyl-2-azaspiro[3.3]heptane-2-carboxylate (250 mg, 914.51 μmol, 1 eq) in DCM (3 mL) was added TFA (312.83 mg, 2.74 mmol, 203.80 μL, 3 eq). The mixture was stirred at 25° C. for 1 h. LCMS showed starting material was consumed and the desired mass was detected. The reaction was filtered and the filtered concentrated under reduced pressure to give compound 7-phenyl-2-azaspiro[3.3]heptane (230 mg, 87.55% yield, TFA) as a yellow oil.

LC-MS [ESI, M+1]:174.1

### Step 4: methyl 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylate

A mixture of 7-phenyl-2-azaspiro[3.3]heptane (223.33 mg, 1.29 mmol, 2 eq), methyl 1-(6-bromo-8-methyl-[1,2,4]triazolo[1,5-a]pyridin-2-yl)-4-[[tert-butyl(diphenyl)silyl]oxymethyl]-2-oxabicyclo[2.1.1]hexane-5-carboxylate (400 mg, 644.53 μmol, 1 eq), Cs₂CO₃ (630.00 mg, 1.93 mmol, 3 eq) and 1,3-bis[2,6-bis(1-propylbutyl)phenyl]-4,5-dichloro-2H-imidazol-1-ium-2-ide;3-chloropyridine;dichloropalladium (31.35 mg, 32.23 μmol, 0.05 eq) in 1,4-dioxane (7 mL) was degassed and purged with N₂ for 3 times, and then the mixture was stirred at 100° C. for 2 h under N₂ atmosphere. LCMS showed starting material was consumed and the desired mass was detected. The reaction was filtered and the filtered concentrated under reduced pressure to give a residue. The residue was purified by flash silica gel chromatography (SiO₂, PE:EtOAc=2:1, Rf=0.3) to give compound methyl 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylate (400 mg, 87.05% yield) as a yellow oil.

LC-MS [ESI, M+1]: 713.5

¹H NMR (400 MHz, CHLOROFORM-d) δ=7.71-7.65 (m, 4H), 7.46 (d, J=1.6 Hz, 1H), 7.44-7.36 (m, 6H), 7.33 (d, J=7.6 Hz, 2H), 7.26-7.19 (m, 3H), 6.61 (d, J=0.8 Hz, 1H), 4.16-4.11 (m, 3H), 4.11-4.07 (m, 1H), 4.05-4.00 (m, 1H), 3.93-3.86 (m, 3H), 3.60-3.57 (m, 4H), 3.55-3.51 (m, 1H), 2.56 (s, 3H), 2.36-2.20 (m, 5H), 2.00 (d, J=7.6 Hz, 1H), 1.08 (s, 9H).

### Step 5:4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylic acid

To a solution of methyl 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylate (400 mg, 561.05 μmol, 1 eq) in THF (3 mL), Water (1 mL) and MeOH (0.5 mL) was added NaOH (44.88 mg, 1.12 mmol, 2 eq). The mixture was stirred at 25° C. for 12 h. LCMS showed starting material was consumed and the desired mass was detected. The reaction was filtered and the filtered concentrated under reduced pressure to give a residue. The crude product was purified by reversed-phase HPLC (0.1% FA condition) to give compound 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylic acid (220 mg, 56.10% yield) as a yellow solid.

LC-MS [ESI, M+1]: 699.6.

### Step 6:4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxamide

To a solution of 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxylic acid (220 mg, 314.77 μmol, 1 eq) and NH₄Cl (101.02 mg, 1.89 mmol, 6 eq) in DMF (3 mL) was added HATU (179.53 mg, 472.16 μmol, 1.5 eq) and DIEA (122.04 mg, 944.31 μmol, 164.48 μL, 3 eq). The mixture was stirred at 25° C. for 12 h. LCMS showed starting material was consumed and the desired mass was detected. The reaction was filtered and the filtered concentrated under reduced pressure to give a residue. The crude product was purified by reversed-phase HPLC (0.1% FA condition) to give compound 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxamide (200 mg, 91.04% yield) as a yellow solid.

LC-MS [ESI, M+1]:699.0

### Step 7: tert-butyl-[[1-[8-methyl-6-(7-phenyl-2-azaspiro[3.3]heptan-2-yl)-[1,2,4]triazolo[1,5-a]pyridin-2-yl]-5-(3-methyl-1H-1,2,4-triazol-5-yl)-2-oxabicyclo[2.1.1]hexan-4-yl]methoxy]-diphenyl-silane

A mixture of 4-[[tert-butyl(diphenyl)silyl]oxymethyl]-1-[8-methyl-6-(7-phenyl-2-azaspiro[3.3]heptan-2-yl)-[1,2,4]triazolo[1,5-a]pyridin-2-yl]-2-oxabicyclo[2.1.1]hexane-5-carboxamide (200 mg, 286.56 μmol, 1 eq) in 1,1-dimethoxy-N,N-dimethylethanamine (190.83 mg, 1.43 mmol, 209.48 μL, 5 eq) was stirred at 110° C. for 0.5 h. The reaction mixture was concentrated in vacuum. Then the solution was added EtOH (2 mL) HOAc (344.17 mg, 5.73 mmol, 328.09 μL, 20 eq) and NH₂NH₂·H₂O (168.77 mg, 2.87 mmol, 163.53 μL, 85% purity, 10 eq). The reaction mixture was stirred at 0° C. for 0.5 h. LCMS showed starting material was consumed and the desired mass was detected. The reaction mixture was added H₂O (3 mL) and the mixture was filtered and the filtrated was concentrated in vacuum. The residue was purified by flash silica gel chromatography (SiO₂, PE:EtOAc=0:1, Rf=0.3) to give compound tert-butyl-[[1-[8-methyl-6-(7-phenyl-2-azaspiro[3.3]heptan-2-yl)-[1,2,4]triazolo[1,5-a]pyridin-2-yl]-5-(3-methyl-1H-1,2,4-triazol-5-yl)-2-oxabicyclo[2.1.1]hexan-4-yl]methoxy]-diphenyl-silane (110 mg, 52.16% yield) as a white solid.

LC-MS [ESI, M+1]: 736.3

¹H NMR (400 MHz, CHLOROFORM-d) δ=7.76-7.61 (m, 4H), 7.50-7.29 (m, 9H), 7.27-7.17 (m, 3H), 6.65 (s, 1H), 4.30 (d, J=11.2 Hz, 1H), 3.91-3.88 (m, 2H), 3.67-3.58 (m, 3H), 3.57-3.51 (m, 1H), 3.42 (s, 1H), 2.56 (s, 3H), 2.40 (s, 3H), 2.37-2.33 (m, 1H), 2.28-2.23 (m, 3H), 1.27 (t, J=7.2 Hz, 4H), 1.07 (s, 9H).

### Step 8: [1-[8-methyl-6-(7-phenyl-2-azaspiro[3.3]heptan-2-yl)-[1,2,4]triazolo[1,5-a]pyridin-2-yl]-5-(3-methyl-1H1,2,4-triazol-5-yl)-2-oxabicyclo[2.1.1]hexan-4-yl]methanol

To a solution of tert-butyl-[[1-[8-methyl-6-(7-phenyl-2-azaspiro[3.3]heptan-2-yl)-[1,2,4]triazolo[1,5-a]pyridin-2-yl]-5-(3-methyl-1H-1,2,4-triazol-5-yl)-2-oxabicyclo[2.1.1]hexan-4-yl]methoxy]-diphenyl-silane (30 mg, 40.76 μmol, 1 eq) in DMSO (1 mL) was added CsF (12.38 mg, 81.52 μmol, 2 eq). The mixture was stirred at 25° C. for 12 h. LCMS showed starting material was consumed and the desired mass was detected. The reaction was filtered and the filtered concentrated under reduced pressure to give a residue. The residue was purified by prep-HPLC (column: CD01-Phenomenex luna C18 150*25*10 um; mobile phase: [H₂O (0.225% FA)—ACN]; gradient:27%-57% B over 10.0 min). The result solution was lyophilized to give compound [1-[8-methyl-6-(7-phenyl-2-azaspiro[3.3]heptan-2-yl)-[1,2,4]triazolo[1,5-a]pyridin-2-yl]-5-(3-methyl-1H1,2,4-triazol-5-yl)-2-oxabicyclo[2.1.1]hexan-4-yl]methanol (7.55 mg, 36.65% yield, 98.463% purity) as a white solid.

LC-MS [ESI, M+1]:498.3

¹H NMR (400 MHz, CHLOROFORM-d) δ=7.43 (s, 1H), 7.39-7.29 (m, 2H), 7.27-7.17 (m, 3H), 6.67 (s, 1H), 4.23 (br d, J=12.0 Hz, 1H), 4.05 (d, J=12.0 Hz, 1H), 3.96-3.88 (m, 2H), 3.85 (s, 2H), 3.66-3.58 (m, 2H), 3.57-3.51 (m, 1H), 3.31 (s, 1H), 2.56 (s, 3H), 2.39 (s, 3H), 2.37-2.19 (m, 6H).

| Compound | | |
|---|---|---|
| Number | Synthesis | LC/MS |
| 578 | Example 85 | m/z 498.3 [M + H]⁺ |
| 579 | Similar to Example 85 | m/z 486.1 [M + H]⁺ |

### Example 86

### ((1R,4S,5R)-5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol (Compound 580)

### ((1S,4R,5R)-5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol (Compound 581)

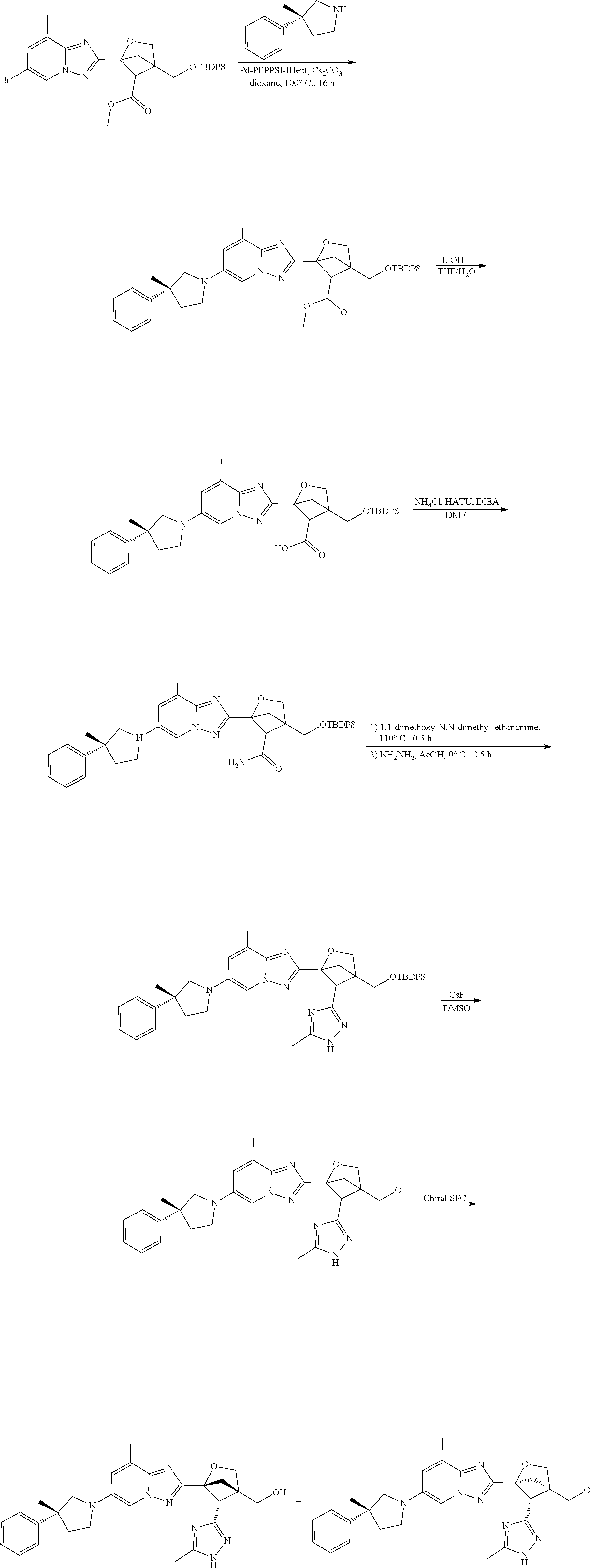

### Step 1: methyl 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylate

A mixture of methyl 1-(6-bromo-8-methyl-[1,2,4]triazolo[1,5-a]pyridin-2-yl)-4-[[tertbutyl(diphenyl)silyl]oxymethyl]-2-oxabicyclo[2.1.1]hexane-5-carboxylate (4 g, 6.45 mmol, 1 eq), (3R)-3-methyl-3-phenyl-pyrrolidine (1.35 g, 8.38 mmol, 1.3 eq), 1,3-bis[2,6-bis(1-propylbutyl)phenyl]-4,5-dichloro-2H-imidazol-1-ium-2-ide;3-chloropyridine;dichloropalladium (313.49 mg, 322.26 μmol, 0.05 eq) and Cs₂CO₃ (5.25 g, 16.11 mmol, 2.5 eq) in dioxane (50 mL) was degassed and purged with N₂ for 3 times, and then the mixture was stirred at 100° C. for 12 h under N₂ atmosphere. LCMS showed starting material was consumed and the desired mass was detected. The reaction mixture was filtered and the filtrate was concentrated under the reduced pressure to give the product. The residue was purified by column chromatography (SiO₂, PE:EtOAc=1:1, Rf=0.50) to give compound methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (4 g, 4.91 mmol, 76.14% yield, 86% purity) as yellow solid.

LC-MS [ESI, M+1]:701.4.

¹H NMR (400 MHz, CHLOROFORM-d) δ=7.69 (dd, J=1.2, 6.4 Hz, 5H), 7.50-7.34 (m, 8H), 7.33-7.28 (m, 2H), 7.26 (br s, 1H), 6.94 (s, 1H), 4.21-4.09 (m, 2H), 4.08-3.99 (m, 1H), 3.93 (d, J=6.0 Hz, 1H), 3.63-3.56 (m, 4H), 3.55-3.44 (m, 2H), 3.43-3.36 (m, 1H), 3.34 (s, 1H), 2.68-2.61 (m, 3H), 2.44-2.30 (m, 2H), 2.29-2.20 (m, 1H), 2.06-2.02 (m, 1H), 1.47 (s, 3H), 1.09 (s, 9H).

### Step 2: 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxylic acid

To a solution of methyl 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylate (2.5 g, 3.57 mmol, 1 eq) in THF (24 mL) and H₂O (8 mL) was added NaOH (427.97 mg, 10.70 mmol, 3 eq) and LiOH·H₂O (74.83 mg, 1.78 mmol, 0.5 eq). The mixture was stirred at 40° C. for 12 h. LCMS showed starting material was consumed and the desired mass was detected. The reaction mixture was concentrated under the reduced pressure to give the crude product. The residue was purified by column chromatography (SiO₂, PE:EtOAc=1:2, Rf=0.50) to give compound 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.8 g, 2.49 mmol, 69.80% yield, 95% purity) as yellow solid.

LC-MS [ESI, M+1]:687.7.

¹H NMR (400 MHz, CHLOROFORM-d) δ=7.72-7.62 (m, 5H), 7.48-7.35 (m, 8H), 7.34-7.28 (m, 3H), 7.04 (s, 1H), 4.31 (d, J=11.2 Hz, 1H), 4.09-4.00 (m, 2H), 3.90 (d, J=6.0 Hz, 1H), 3.62 (br d, J=9.2 Hz, 1H), 3.57-3.46 (m, 2H), 3.45-3.35 (m, 1H), 3.18 (s, 1H), 2.60 (s, 3H), 2.45-2.36 (m, 1H), 2.33-2.23 (m, 1H), 2.18 (d, J=4.0 Hz, 1H), 2.06 (s, 1H), 1.48 (s, 3H), 1.07 (s, 9H).

### Step 3: 4-(((tert-butyldiphenylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexane-5-carboxamide

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxylic acid (1.1 g, 1.60 mmol, 1 eq) in DMF (5 mL) was added NH₄Cl (342.64 mg, 6.41 mmol, 4 eq), DIEA (620.89 mg, 4.80 mmol, 836.79 μL, 3 eq) and HATU (1.22 g, 3.20 mmol, 2 eq). The mixture was stirred at 25° C. for 12 h. LCMS showed starting material was consumed and the desired mass was detected. The mixture was quenched with water (30 mL) and extracted with ethyl acetate (50 mL×2). The combined organic phase was washed with brine (80 mL), dried with anhydrous Na₂SO₄, filtered and concentrated in vacuum. The crude product was purified by reversed-phase HPLC (0.1% FA condition) to give compound 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (400 mg, 571.49 μmol, 35.69% yield, 98% purity) as white solid.

LC-MS [ESI, M+1]:686.3.

¹H NMR (400 MHz, CHLOROFORM-d) δ=8.56 (br s, 1H), 7.74-7.63 (m, 5H), 7.47-7.35 (m, 8H), 7.34-7.30 (m, 2H), 7.29 (br d, J=1.2 Hz, 1H), 7.25 (s, 1H), 5.49 (br s, 1H), 4.29 (d, J=11.2 Hz, 1H), 4.12 (d, J=11.2 Hz, 1H), 4.04-3.91 (m, 2H), 3.60 (d, J=8.8 Hz, 1H), 3.56-3.45 (m, 2H), 3.43-3.35 (m, 1H), 3.07 (s, 1H), 2.61 (s, 3H), 2.39 (td, J=8.4, 12.4 Hz, 1H), 2.31-2.16 (m, 3H), 1.47 (s, 3H), 1.08 (s, 9H)

### Step 4: 2-(4-(((tert-butyldiphenylsilyl)oxy)methyl)-5-(5-methyl-11H-1,2,4-triazol-3-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of 4-[[tert-butyl(diphenyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexane-5-carboxamide (720 mg, 1.05 mmol, 1 eq) in 1,1-dimethoxy-N,N-dimethyl-ethanamine (8.25 g, 61.94 mmol, 9.06 mL, 59.01 eq), the mixture was stirred at 110° C. for 0.5 h. Then the mixture was concentrated under reduced pressure to give a residue. To a solution of this residue in EtOH (10 mL) was added NH₂NH₂·H₂O (618.20 mg, 10.50 mmol, 599.03 μL, 85% purity, 10 eq) and HOAc (1.26 g, 20.99 mmol, 1.20 mL, 20 eq) at 0° C. And then the mixture was stirred at 0° C. for 0.5 h. LCMS showed starting material was consumed and the desired mass was detected. The mixture was quenched with water (20 mL) and extracted with ethyl acetate (30 mL×2). The combined organic phase was washed with brine (30 mL), dried with anhydrous Na₂SO₄, filtered and concentrated in vacuum. The residue was purified by prep-TLC (SiO₂, PE:EtOAc=0:1, Rf=0.50) to give compound tert-butyl-[[1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methoxy]-diphenyl-silane (500 mg, 683.72 μmol, 65.14% yield, 99% purity) as red oil.

LC-MS [ESI, M+1]:724.3.

¹H NMR (400 MHz, CHLOROFORM-d) δ=7.79-7.58 (m, 5H), 7.48-7.35 (m, 7H), 7.35-7.27 (m, 3H), 7.27-7.21 (m, 1H), 7.00 (s, 1H), 4.33 (d, J=11.2 Hz, 1H), 4.18 (d, J=11.2 Hz, 1H), 3.92 (d, J=6.0 Hz, 1H), 3.69-3.58 (m, 2H), 3.57-3.44 (m, 3H), 3.40 (dt, J=3.6, 8.8 Hz, 1H), 2.64 (s, 3H), 2.42-2.34 (m, 2H), 2.33-2.20 (m, 2H), 2.05 (s, 3H), 1.47 (s, 3H), 1.08 (s, 9H)

### Step 5: (5-(5-methyl-1H-1,2,4-triazol-3-yl)-1-(6-((R)-3-methyl-3-phenylpyrrolidin-1-yl)-[1,2,4]triazolo[1,5-a]pyridin-2-yl)-2-oxabicyclo[2.1.1]hexan-4-yl)methanol

To a solution of tert-butyl-[[1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methoxy]-diphenyl-silane (700 mg, 966.88 μmol, 1 eq) in DMSO (8 mL) was added CsF (293.74 mg, 1.93 mmol, 2 eq). The mixture was stirred at 25° C. for 12 h. LCMS showed starting material was consumed and the desired mass was detected. The reaction mixture was filtered and the filtrate was concentrated under the reduced pressure to give the product. The residue was purified by prep-HPLC (FA condition column: CD05-Phenomenex luna C18 150*40*10 um; mobile phase: [water(FA)—ACN]; gradient:22%-52% B over 10 min) to give compound [1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methanol (300 mg, 614.73 μmol, 63.58% yield, 99.5% purity) as white solid.

LC-MS [ESI, M+1]:486.1.

¹H NMR (400 MHz, CHLOROFORM-d) δ=7.65 (d, J=1.6 Hz, 1H), 7.40-7.35 (m, 2H), 7.34-7.27 (m, 3H), 7.02 (s, 1H), 5.31 (s, 1H), 4.28 (d, J=12.0 Hz, 1H), 4.08 (d, J=12.0 Hz, 1H), 3.93-3.83 (m, 2H), 3.61 (d, J=8.8 Hz, 1H), 3.58-3.46 (m, 2H), 3.41 (dt, J=3.6, 8.8 Hz, 1H), 3.34 (s, 1H), 2.65 (s, 3H), 2.42 (s, 3H), 2.41-2.33 (m, 2H), 2.33-2.23 (m, 2H), 1.47 (s, 3H).

### Step 6: ((1R,4S,5R)-5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol

The residue was purified by prep-SFC (column: DAICEL CHIRALCEL OX (250 mm*30 mm, 10 um);mobile phase: [C02-ACN/MeOH (0.1% NH₃H₂O)]; B %: 70%, isocratic elution mode) and lyophilized to give compound [(1R,4S,5R)-1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methanol (133.15 mg, 271.61 μmol, 43.96% yield, 99.053% purity) as white solid.

LC-MS [ESI, M+1]:486.2.

¹H NMR (400 MHz, CHLOROFORM-d) δ=7.65 (d, J=1.6 Hz, 1H), 7.41-7.34 (m, 2H), 7.34-7.29 (m, 2H), 7.28 (br s, 1H), 7.02 (s, 1H), 4.28 (d, J=12.0 Hz, 1H), 4.08 (d, J=12.0 Hz, 1H), 3.97-3.81 (m, 2H), 3.61 (d, J=8.8 Hz, 1H), 3.57-3.46 (m, 2H), 3.40 (dt, J=3.6, 8.8 Hz, 1H), 3.33 (s, 1H), 2.65 (s, 3H), 2.42 (s, 3H), 2.41-2.32 (m, 2H), 2.32-2.22 (m, 2H), 1.47 (s, 3H)

### Step 6: ((1S,4R,5R)-5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanol

The residue was purified by prep-SFC (column: DAICEL CHIRALCEL OX (250 mm*30 mm, 10 um);mobile phase: [C02-ACN/MeOH (0.1% NH₃H₂O)]; B %: 70%, isocratic elution mode) and lyophilized to give compound [(1S,4R)-1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methanol (133.66 mg, 275.26 μmol, 44.55% yield, 100% purity) was obtained as a white solid.

LC-MS [ESI, M+1]:486.2.

¹H NMR (400 MHz, CHLOROFORM-d) δ=7.65 (d, J=1.6 Hz, 1H), 7.42-7.35 (m, 2H), 7.34-7.27 (m, 3H), 7.02 (s, 1H), 4.28 (d, J=12.0 Hz, 1H), 4.08 (d, J=12.0 Hz, 1H), 3.95-3.83 (m, 2H), 3.61 (d, J=8.8 Hz, 1H), 3.58-3.47 (m, 2H), 3.41 (dt, J=3.6, 8.8 Hz, 1H), 3.34 (s, 1H), 2.65 (s, 3H), 2.42 (s, 3H), 2.41-2.33 (m, 2H), 2.32-2.23 (m, 2H), 1.48 (s, 3H).

### Example 87

### N-((5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)ethanesulfonamide (Compound 583)

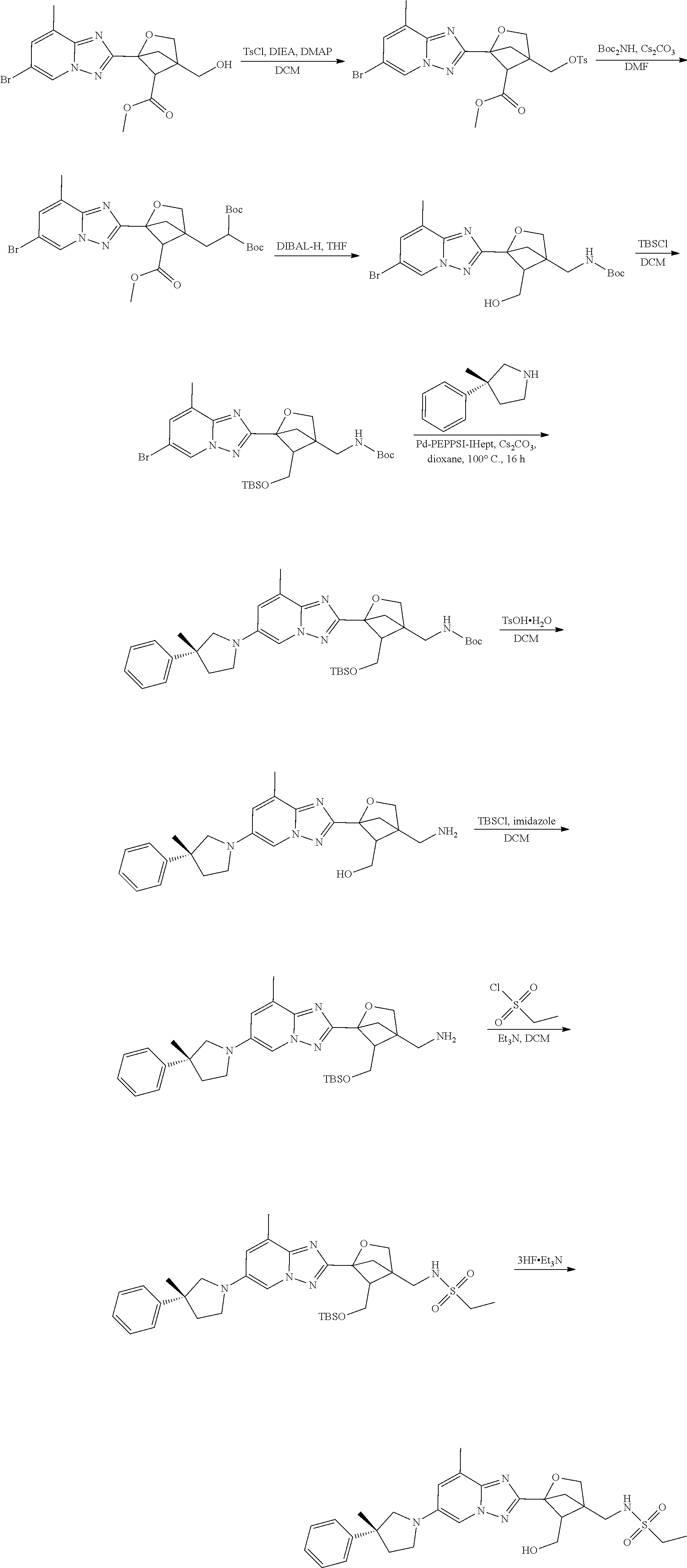

### Step 1: methyl 1-(6-bromo-8-methyl-[1,2,4]triazolo[1,5-a]pyridin-2-yl)-4-((tosyloxy)methyl)-2-oxabicyclo[2.1.1]hexane-5-carboxylate

To a solution of methyl 1-(6-bromo-8-methyl-[1,2,4]triazolo[1,5-a]pyridin-2-yl)-4-(hydroxymethyl)-2-oxabicyclo[2.1.1]hexane-5-carboxylate (2.4 g, 6.28 mmol, 1 eq) in DCM (30 mL) was added DMAP (76.71 mg, 627.93 μmol, 0.1 eq), TosCl (3.59 g, 18.84 mmol, 3 eq) and TEA (1.91 g, 18.84 mmol, 2.62 mL, 3 eq). The mixture was stirred at 25° C. for 12 h. LCMS showed starting material was consumed and the desired mass was detected. The reaction was added water (25 mL) and then extracted with DCM (25 mL×3). The combined organic phase was washed with brine (20 mL×2), dried over anhydrous Na₂SO₄, filtered and concentrated in vacuo. The residue was purified by column chromatography (SiO₂, PE:EtOAc=1:1, Rf=0.50) to give compound methyl 1-(6-bromo-8-methyl-[1,2,4]triazolo[1,5-a]pyridin-2-yl)-4-(p-tolylsulfonyloxymethyl)-2-oxabicyclo[2.1.1]hexane-5-carboxylate (2 g, 3.73 mmol, 59.38% yield) as yellow oil.

LC-MS [ESI, M+1]:536.1.

¹H NMR (400 MHz, CHLOROFORM-d) δ=8.57 (d, J=0.4 Hz, 1H), 7.85 (d, J=8.4 Hz, 2H), 7.49-7.34 (m, 3H), 4.62-4.51 (m, 1H), 4.49-4.41 (m, 1H), 4.08 (d, J=6.0 Hz, 1H), 3.93 (d, J=6.4 Hz, 1H), 3.58 (s, 3H), 3.33 (s, 1H), 2.67 (s, 3H), 2.49 (s, 3H), 2.37 (d, J=7.6 Hz, 1H), 2.01 (d, J=7.6 Hz, 1H)

### Step 2: methyl 4-((bis(tert-butoxycarbonyl)amino)methyl)-1-(6-bromo-8-methyl-[1,2,4]triazolo[1,5-a]pyridin-2-yl)-2-oxabicyclo[2.1.1]hexane-5-carboxylate

To a solution of methyl 1-(6-bromo-8-methyl-[1,2,4]triazolo[1,5-a]pyridin-2-yl)-4-(p-tolylsulfonyloxymethyl)-2-oxabicyclo[2.1.1]hexane-5-carboxylate (2 g, 3.73 mmol, 1 eq) in 1,3-dimethylimidazolidin-2-one (20 mL) was added Cs₂CO₃ (3.64 g, 11.19 mmol, 3 eq) and tert-butyl N-tert-butoxycarbonylcarbamate (1.62 g, 7.46 mmol, 2 eq). The mixture was stirred at 75° C. for 1 h. LCMS showed starting material was consumed and the desired mass was detected. The mixture was quenched with water (60 mL) and extracted with ethyl acetate (80 mL×2). The combined organic phase was washed with brine (200 mL), dried with anhydrous Na₂SO₄, filtered and concentrated in vacuum. The crude product was purified by reversed-phase HPLC (0.1% FA condition) to give compound methyl 4-[[bis(tert-butoxycarbonyl)amino]methyl]-1-(6-bromo-8-methyl-[1,2,4]triazolo[1,5-a]pyridin-2-yl)-2-oxabicyclo[2.1.1]hexane-5-carboxylate (1.5 g, 2.32 mmol, 62.27% yield, 90% purity) as yellow solid.

LC-MS [ESI, M+1]: 583.3.

¹H NMR (400 MHz, CHLOROFORM-d) δ=8.56 (d, J=0.8 Hz, 1H), 7.43-7.35 (m, 1H), 4.56-4.33 (m, 1H), 4.31 (d, J=6.4 Hz, 1H), 4.15-4.09 (m, 1H), 3.95-3.88 (m, 1H), 3.60 (s, 3H), 3.24 (s, 1H), 2.66 (s, 3H), 2.22 (d, J=7.6 Hz, 1H), 2.01 (d, J=8.0 Hz, 1H), 1.52 (s, 18H).

### Step 3: tert-butyl ((1-(6-bromo-8-methyl-[1,2,4]triazolo[1,5-a]pyridin-2-yl)-5-(hydroxymethyl)-2-oxabicyclo[2.1.1]hexan-4-yl)methyl)carbamate

A mixture of methyl 4-[[bis(tert-butoxycarbonyl)amino]methyl]-1-(6-bromo-8-methyl-[1,2,4]triazolo[1,5-a]pyridin-2-yl)-2-oxabicyclo[2.1.1]hexane-5-carboxylate (1 g, 1.72 mmol, 1 eq) and DIBAL-H (1 M, 5.16 mL, 3 eq) in THF (12 mL) was degassed and purged with N₂ for 3 times, and then the mixture was stirred at −78° C. for 2 h under N₂ atmosphere. LCMS showed starting material was consumed and the desired mass was detected. The reaction was added water (15 mL) and then extracted with ethyl acetate (30 mL×3). The combined organic phase was washed with brine (20 mL×2), dried over anhydrous Na₂SO₄, filtered and concentrated in vacuum The residue was purified by column chromatography (SiO₂, PE:EtOAc=0:1, Rf=0.50) to give compound tert-butyl ((1-(6-bromo-8-methyl-[1,2,4]triazolo[1,5-a]pyridin-2-yl)-5-(hydroxymethyl)-2-oxabicyclo[2.1.1]hexan-4-yl)methyl)carbamate (250 mg, 512.87 μmol, 29.82% yield, 93% purity) as colorless oil.

LC-MS [ESI, M+1]:453.2.

### Step 4: tert-butyl ((1-(6-bromo-8-methyl-[1,2,4]triazolo[1,5-a]pyridin-2-yl)-5-(((tert-butyldimethylsilyl)oxy)methyl)-2-oxabicyclo[2.1.1]hexan-4-yl)methyl)carbamate

To a solution of tert-butyl N-[[1-(6-bromo-8-methyl-[1,2,4]triazolo[1,5-a]pyridin-2-yl)-5-(hydroxymethyl)-2-oxabicyclo[2.1.1]hexan-4-yl]methyl]carbamate (200 mg, 441.18 μmol, 1 eq) in DCM (4 mL) was added TBSCl (132.99 mg, 882.36 μmol, 108.56 μL, 2 eq) and imidazole (60.07 mg, 882.36 μmol, 2 eq). The mixture was stirred at 0° C. for 12 h. LCMS showed starting material was consumed and the desired mass was detected. The reaction mixture was concentrated under the reduced pressure to give the crude product. The residue was purified by column chromatography (SiO₂, PE:EtOAc=1:1, Rf=0.50) to give compound tert-butyl ((1-(6-bromo-8-methyl-[1,2,4]triazolo[1,5-a]pyridin-2-yl)-5-(((tert-butyldimethylsilyl)oxy)methyl)-2-oxabicyclo[2.1.1]hexan-4-yl)methyl)carbamate (230 mg, 364.70 μmol, 82.66% yield, 90% purity) as colorless oil.

LC-MS [ESI, M+1]:567.3.

¹H NMR (400 MHz, CHLOROFORM-d) δ=8.53 (s, 1H), 7.39 (s, 1H), 5.59 (br d, J=5.6 Hz, 1H), 4.01-3.86 (m, 2H), 3.77-3.63 (m, 3H), 3.41 (br dd, J=2.0, 14.0 Hz, 1H), 2.64 (s, 3H), 2.50 (br d, J=6.8 Hz, 1H), 2.11-2.06 (m, 2H), 1.46 (s, 9H), 0.89 (s, 9H), 0.08 (d, J=4.4 Hz, 6H).

### Step 5: tert-butyl ((5-(((tert-butyldimethylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)carbamate

A mixture of tert-butyl N-[[1-(6-bromo-8-methyl-[1,2,4]triazolo[1,5-a]pyridin-2-yl)-5-[[tertbutyl(dimethyl)silyl]oxymethyl]-2-oxabicyclo[2.1.1]hexan-4-yl]methyl]carbamate (230 mg, 405.22 μmol, 1 eq), (3R)-3-methyl-3-phenyl-pyrrolidine (71.87 mg, 445.74 μmol, 1.1 eq), Cs₂CO₃ (330.07 mg, 1.01 mmol, 2.5 eq) and 1,3-bis[2,6-bis(1-propylbutyl)phenyl]-4,5-dichloro-2H-imidazol-1-ium-2-ide;3-chloropyridine;dichloropalladium (19.71 mg, 20.26 μmol, 0.05 eq) in dioxane (4 mL) was degassed and purged with N₂ for 3 times, and then the mixture was stirred at 100° C. for 12 h under N₂ atmosphere. LCMS showed starting material was consumed and the desired mass was detected. The reaction mixture was concentrated under the reduced pressure to give the crude product. The residue was purified by column chromatography (SiO₂, PE:EtOAc=1:1, Rf=0.50) to give compound tert-butyl N-[[5-[[tert-butyl(dimethyl)silyl]oxymethyl]-1-[8-methyl-6-[(3R)-3-methyl-3-phenyl-pyrrolidin1-yl]-[1,2,4]triazolo[1,5-a]pyridin-2-yl]-2-oxabicyclo[2.1.1]hexan-4-yl]methyl]carbamate (200 mg, 246.94 μmol, 60.94% yield, 80% purity) as yellow oil.

LC-MS [ESI, M+1]:648.6.

### Step 6: (4-(aminomethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)methanol (Compound 582)

To a solution of tert-butyl N-[[5-[[tert-butyl(dimethyl)silyl]oxymethyl]-1-[8-methyl-6-[(3R)-3-methyl-3-phenylpyrrolidin-1-yl]-[1,2,4]triazolo[1,5-a]pyridin-2-yl]-2-oxabicyclo[2.1.1]hexan-4-yl]methyl]carbamate (200 mg, 308.68 μmol, 1 eq) in MeOH (1.5 mL) and ACN (0.5 mL) was added TsOH·H₂O (234.86 mg, 1.23 mmol, 4 eq). The mixture was stirred at 25° C. for 4 h. LCMS showed starting material was consumed and the desired mass was detected. The reaction mixture was concentrated under the reduced pressure to give the crude product. The crude product was purified by reversed-phase HPLC (0.1% NH₃·H₂O condition) to give compound [4-(aminomethyl)-1-[8-methyl-6-[(3R)-3-methyl-3-phenyl-pyrrolidin-1-yl]-[1,2,4]triazolo[1,5-a]pyridin2-yl]-2-oxabicyclo[2.1.1]hexan-5-yl]methanol (130 mg, 239.88 μmol, 77.71% yield, 80% purity) as yellow solid.

LC-MS [ESI, M+1]:434.3.

¹H NMR (400 MHz, CHLOROFORM-d) δ=7.62 (s, 1H), 7.40-7.33 (m, 2H), 7.33-7.29 (m, 2H), 7.27-7.22 (m, 1H), 6.94 (s, 1H), 3.94 (d, J=6.0 Hz, 1H), 3.86-3.75 (m, 2H), 3.65 (dd, J=7.2, 11.6 Hz, 1H), 3.59 (d, J=8.8 Hz, 1H), 3.54-3.42 (m, 2H), 3.38 (dt, J=3.6, 8.8 Hz, 1H), 3.19 (d, J=13.2 Hz, 1H), 3.03 (d, J=13.6 Hz, 1H), 2.60 (s, 3H), 2.51 (t, J=7.2 Hz, 1H), 2.37 (td, J=8.4, 12.0 Hz, 1H), 2.28-2.21 (m, 1H), 2.14 (br d, J=7.6 Hz, 1H), 2.04-2.03 (m, 1H), 1.46 (s, 3H).

### Step 7: (5-(((tert-butyldimethylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanamine

To a solution of [4-(aminomethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-5-yl]methanol (110 mg, 253.72 μmol, 1 eq) in DCM (3 mL) was added TBSCl (76.48 mg, 507.44 μmol, 62.44 μL, 2 eq) and imidazole (34.55 mg, 507.44 μmol, 2 eq). The mixture was stirred at 25° C. for 4 h. LCMS showed starting material was consumed and the desired mass was detected. The reaction mixture was concentrated under the reduced pressure to give the crude product. The residue was purified by column chromatography (SiO₂, DCM:MeOH=10:1, Rf=0.50) to give compound [5-[[tert-butyl(dimethyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanamine (130 mg, 166.12 μmol, 65.47% yield, 70% purity) as yellow oil.

LC-MS [ESI, M+1]:548.5.

¹H NMR (400 MHz, CHLOROFORM-d) δ=7.77 (d, J=1.6 Hz, 1H), 7.55-7.48 (m, 2H), 7.48-7.44 (m, 2H), 7.41-7.37 (m, 1H), 7.08 (s, 1H), 4.11 (d, J=6.4 Hz, 1H), 4.03 (d, J=6.4 Hz, 1H), 3.92-3.85 (m, 2H), 3.73 (d, J=8.8 Hz, 1H), 3.64-3.58 (m, 2H), 3.53 (dt, J=3.8, 8.8 Hz, 1H), 3.32-3.18 (m, 2H), 2.76 (s, 3H), 2.69 (t, J=7.2 Hz, 1H), 2.51 (td, J=8.4, 12.0 Hz, 1H), 2.44-2.35 (m, 1H), 2.26 (d, J=7.2 Hz, 1H), 2.13 (d, J=7.2 Hz, 1H), 1.61 (s, 3H), 1.01 (s, 9H), 0.18 (d, J=3.6 Hz, 6H).

### Step 8: N-((5-(((tert-butyldimethylsilyl)oxy)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)ethanesulfonamide

To a solution of [5-[[tert-butyl(dimethyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methanamine (45 mg, 82.15 μmol, 1 eq) in DCM (0.5 mL) was added TEA (41.56 mg, 410.73 μmol, 57.17 μL, 5 eq) and ethanesulfonyl chloride (52.81 mg, 410.73 μmol, 38.92 μL, 5 eq).The mixture was stirred at 0° C. for 15 min. LCMS showed starting material was consumed and the desired mass was detected. The reaction mixture was concentrated under the reduced pressure to give the crude product. The residue was purified by column chromatography (SiO₂, PE:EtOAc=0:1, Rf=0.70) to give compound N-[[5-[[tert-butyl(dimethyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]ethanesulfonamide (30 mg, 42.19 μmol, 51.36% yield, 90% purity) as yellow oil.

LC-MS [ESI, M+1]:640.3.

### Step 9: N-((5-(hydroxymethyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)ethanesulfonamide

To a solution of N-[[5-[[tert-butyl(dimethyl)silyl]oxymethyl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]ethanesulfonamide (30 mg, 46.88 μmol, 1 eq) in DCM (0.5 mL) was added N,N-diethylethanamine;trihydrofluoride (22.67 mg, 140.64 μmol, 22.92 μL, 3 eq). The mixture was stirred at 25° C. for 0.5 h. LCMS showed starting material was consumed and the desired mass was detected. The reaction mixture was concentrated under the reduced pressure to give the crude product. The residue was purified by prep-HPLC (FA condition column: CD01-Phenomenex luna C18 150*25*10 um; mobile phase: [H₂O (0.225% FA)—ACN]; gradient:32%-62% B over 10.0 min) to give compound N-[[5-(hydroxymethyl)-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methyl]ethanesulfonamide (9 mg, 17.12 μmol, 36.52% yield, 100% purity) as yellow solid.

LC-MS [ESI, M+1]:526.3.

¹H NMR (400 MHz, CHLOROFORM-d) δ=7.61 (d, J=1.6 Hz, 1H), 7.39-7.33 (m, 2H), 7.33-7.28 (m, 2H), 7.27-7.22 (m, 1H), 6.95 (s, 1H), 5.36 (br t, J=5.6 Hz, 1H), 4.00-3.86 (m, 3H), 3.68 (dd, J=6.8, 11.2 Hz, 1H), 3.62-3.52 (m, 2H), 3.52-3.43 (m, 3H), 3.42-3.34 (m, 1H), 3.07 (q, J=7.6 Hz, 2H), 2.59 (s, 3H), 2.52 (t, J=6.8 Hz, 1H), 2.37 (td, J=8.4, 12.0 Hz, 1H), 2.29-2.22 (m, 1H), 2.19 (d, J=7.6 Hz, 1H), 2.08 (d, J=7.6 Hz, 1H), 1.46 (s, 3H), 1.39 (t, J=7.6 Hz, 3H).

### Example 88

### 2,2-dimethyl-3-(N-((5-(5-methyl-11H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)sulfamoyl)propanoic acid (Compound 584)

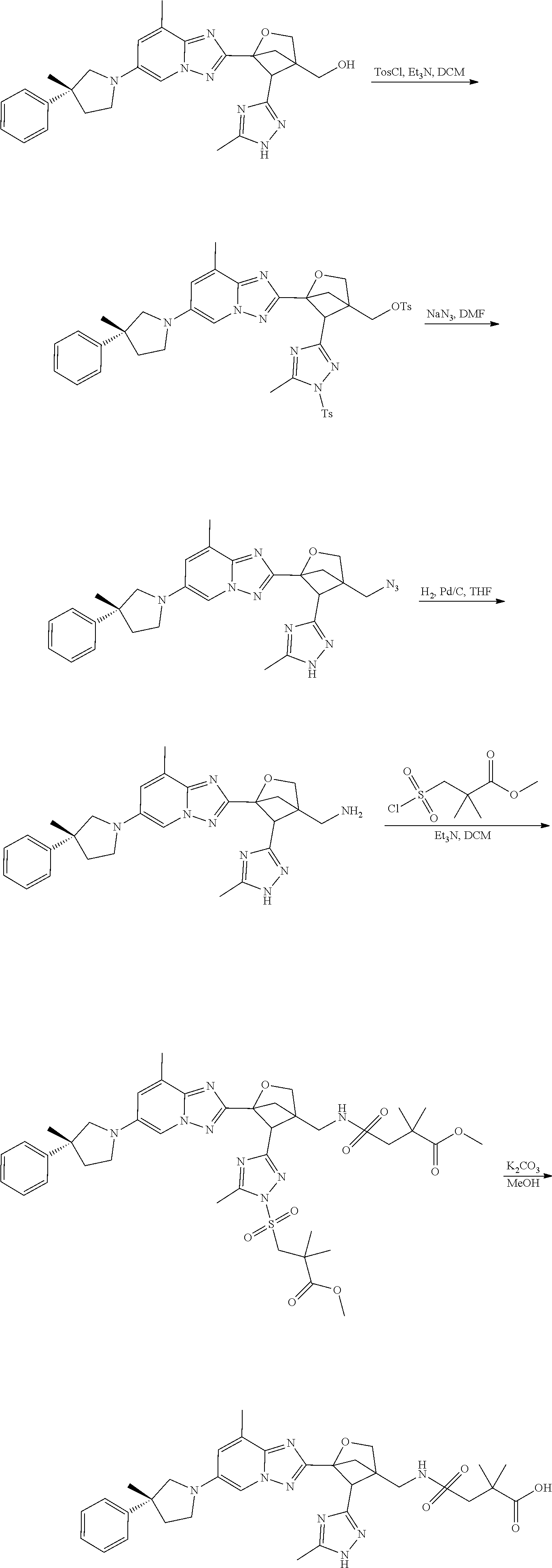

### Step 1: (5-(5-methyl-1-tosyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl 4-methylbenzenesulfonate

To a solution of [1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methanol (1.2 g, 2.47 mmol, 1 eq) in DCM (20 mL) was added DMAP (30.19 mg, 247.13 μmol, 0.1 eq), TosCl (2.36 g, 12.36 mmol, 5 eq) and TEA (750.20 mg, 7.41 mmol, 1.03 mL, 3 eq). The mixture was stirred at 0° C. for 2 h. LCMS showed starting material was consumed and the desired mass was detected. The reaction mixture was concentrated under the reduced pressure to give the crude product. The residue was purified by column chromatography (SiO₂, PE:EtOAc=0:1, Rf=0.4) to give compound [1-[8-methyl-6-[(3R)-3-methyl-3-phenyl-pyrrolidin-1-yl]-[1,2,4]triazolo[1,5-a]pyridin-2-yl]-5-[5-methyl1-(p-tolylsulfonyl)-1,2,4-triazol-3-yl]-2-oxabicyclo[2.1.1]hexan-4-yl]methyl 4-methylbenzenesulfonate (1.4 g, 1.68 mmol, 67.79% yield, 95% purity) as yellow solid.

LC-MS [ESI, M+1]:794.3.

¹H NMR (400 MHz, CHLOROFORM-d) δ=7.86 (dd, J=8.4, 13.6 Hz, 4H), 7.63 (d, J=1.6 Hz, 1H), 7.37 (br d, J=8.0 Hz, 5H), 7.34-7.30 (m, 3H), 7.27 (br s, 1H), 6.93 (s, 1H), 4.65-4.51 (m, 2H), 4.07 (d, J=6.0 Hz, 1H), 3.84 (d, J=6.0 Hz, 1H), 3.59 (d, J=8.8 Hz, 1H), 3.55-3.43 (m, 3H), 3.38 (dt, J=3.6, 8.8 Hz, 1H), 2.61 (d, J=2.8 Hz, 6H), 2.46 (d, J=6.0 Hz, 6H), 2.42-2.31 (m, 2H), 2.30-2.22 (m, 1H), 2.08 (d, J=8.4 Hz, 1H), 1.47 (s, 3H).

### Step 2: 2-(4-(azidomethyl)-5-(5-methyl-1H-1,2,4-triazol-3-yl)-2-oxabicyclo[2.1.1]hexan-1-yl)-8-methyl-6-((R)-3-methyl-3-phenylpyrrolidin-1-yl)-[1,2,4]triazolo[1,5-a]pyridine

To a solution of [1-[8-methyl-6-[(3R)-3-methyl-3-phenyl-pyrrolidin-1-yl]-[1,2,4]triazolo[1,5-a]pyridin-2-yl]-5-[5-methyl-1-(p-tolylsulfonyl)-1,2,4-triazol-3-yl]-2-oxabicyclo[2.1.1]hexan-4-yl]methyl 4-methylbenzenesulfonate (1 g, 1.26 mmol, 1 eq) in DMF (10 mL) was added NaN₃ (327.53 mg, 5.04 mmol, 4 eq). The mixture was stirred at 25° C. for 1 h. LCMS showed starting material was consumed and the desired mass was detected. The mixture was quenched with water (30 mL) and extracted with ethyl acetate (50 mL×2). The combined organic phase was washed with brine (50 mL), dried with anhydrous Na₂SO₄, filtered and concentrated in vacuum to give compound 2-[4-(azidomethyl)-5-(5-methyl-1H-1,2,4-triazol-3-yl)-2-oxabicyclo[2.1.1]hexan-1-yl]-8-methyl-6-[(3R)-3-methyl-3-phenyl-pyrrolidin-1-yl]-[1,2,4]triazolo[1,5-a]pyridine (800 mg, crude) as yellow oil.

LC-MS [ESI, M+1]:511.4.

### Step 3: (5-(5-methyl-11H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methanamine

To a solution of 2-[4-(azidomethyl)-5-(5-methyl-1H-1,2,4-triazol-3-yl)-2-oxabicyclo[2.1.1]hexan-1-yl]-8-methyl-6-[(3R)-3-methyl-3-phenyl-pyrrolidin-1-yl]-[1,2,4]triazolo[1,5-a]pyridine (700 mg, 1.37 mmol, 1 eq) in THF (20 mL) was added Pd/C (145.90 mg, 137.10 μmol, 10% purity, 0.1 eq) under N₂ atmosphere. The suspension was degassed and purged with H₂ for 3 times. The mixture was stirred under H₂ (50 Psi) at 25° C. for 3 h. LCMS showed starting material was consumed and the desired mass was detected. The reaction mixture was concentrated under the reduced pressure to give the crude product. The crude product was purified by reversed-phase HPLC (0.1% FA condition) to give compound [1-[8-methyl-6-[(3R)-3-methyl-3-phenyl-pyrrolidin-1-yl]-[1,2,4]triazolo[1,5-a]pyridin-2-yl]-5-(5-methyl1H-1,2,4-triazol-3-yl)-2-oxabicyclo[2.1.1]hexan-4-yl]methanamine (340 mg, 631.46 μmol, 46.06% yield, 90% purity) as yellow solid.

LC-MS [ESI, M+1]:485.3.

¹H NMR (400 MHz, CHLOROFORM-d) δ=8.53 (s, 1H), 7.81 (d, J=8.4 Hz, 2H), 7.65 (d, J=1.6 Hz, 1H), 7.40-7.34 (m, 2H), 7.33-7.29 (m, 3H), 7.00 (s, 1H), 3.90 (d, J=6.8 Hz, 1H), 3.83 (d, J=6.8 Hz, 1H), 3.61-3.59 (m, 1H), 3.59 (br s, 1H), 3.56-3.49 (m, 1H), 3.46 (s, 1H), 3.39 (dt, J=3.6, 8.8 Hz, 1H), 2.63 (s, 3H), 2.42 (s, 3H), 2.39 (s, 4H), 2.38-2.35 (m, 1H), 2.31-2.20 (m, 1H), 1.46 (s, 3H).

### Step 4: methyl 3-((3-(4-(((3-methoxy-2,2-dimethyl-3-oxopropyl)sulfonamido)methyl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-5-yl)-5-methyl-1H-1,2,4-triazol-1-yl)sulfonyl)-2,2-dimethylpropanoate

To a solution of [1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methanamine (20 mg, 41.27 μmol, 1 eq) in DCM (0.5 mL) was added TEA (20.88 mg, 206.36 μmol, 28.72 μL, 5 eq) and methyl 3-chlorosulfonyl-2,2-dimethyl-propanoate (44.30 mg, 206.36 μmol, 5 eq). The mixture was stirred at 0° C. for 0.5 h. LCMS showed starting material was consumed and the desired mass was detected. The reaction mixture was concentrated under the reduced pressure to give compound methyl 3-[[5-[1-(3-methoxy-2,2-dimethyl-3-oxo-propyl)sulfonyl-5-methyl-1,2,4-triazol-3-yl]-1-[8-methyl6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methylsulfamoyl]-2,2-dimethyl-propanoate (30 mg, 35.67 μmol, 86.43% yield) as white solid.

LC-MS [ESI, M+1]:841.4.

### Step 5: 2,2-dimethyl-3-(N-((5-(5-methyl-1H-1,2,4-triazol-3-yl)-1-(8-methyl-6-((R)-3-methyl-3-phenylpyrrolidin-1-yl)-[1,2,4]triazolo[1,5-a]pyridin-2-yl)-2-oxabicyclo[2.1.1]hexan-4-yl)methyl)sulfamoyl)propanoic acid

To a solution of methyl 3-[[5-[1-(3-methoxy-2,2-dimethyl-3-oxo-propyl)sulfonyl-5-methyl-1,2,4-triazol-3-yl]-1-[8-methyl-6-[(3R)-3-methyl-3-phenyl-pyrrolidin-1-yl]-[1,2,4]triazolo[1,5-a]pyridin-2-yl]-2-oxabicyclo[2.1.1]hexan-4-yl]methylsulfamoyl]-2,2-dimethyl-propanoate (30 mg, 35.67 μmol, 1 eq) in THF (0.6 mL) and H₂O (0.2 mL) was added LiOH·H₂O (4.49 mg, 107.01 μmol, 3 eq). The mixture was stirred at 25° C. for 0.5 h. LCMS showed starting material was consumed and the desired mass was detected. The reaction mixture was concentrated under the reduced pressure to give the crude product. The residue was purified by prep-HPLC (neutral condition column: CD02-Waters Xbidge BEH C18 150*25*10 um; mobile phase: [water(NH₄HCO₃)—ACN]; gradient:16%-46% B over 10 min) to give compound 2,2-dimethyl-3-[[1-[8-methyl-6-[(3R)-3-methyl-3-phenyl-pyrrolidin-1-yl]-[1,2,4]triazolo[1,5-a]pyridin-2-yl]-5-(5-methyl-1H-1,2,4-triazol-3-yl)-2-oxabicyclo[2.1.1]hexan-4-yl]methylsulfamoyl]propanoic acid (15.41 mg, 23.68 μmol, 66.37% yield, 99.68% purity) as white solid.

LC-MS [ESI, M+1]:649.3.

¹H NMR (400 MHz, METHANOL-d4) δ=7.74 (s, 1H), 7.42-7.28 (m, 4H), 7.27-7.13 (m, 2H), 3.99-3.85 (m, 2H), 3.81-3.67 (m, 1H), 3.61 (d, J=3.6 Hz, 2H), 3.59-3.50 (m, 3H), 3.46 (d, J=2.0 Hz, 1H), 3.39 (dt, J=4.0, 8.8 Hz, 1H), 3.18 (s, 1H), 2.58 (s, 3H), 2.43-2.25 (m, 6H), 2.10 (br d, J=7.6 Hz, 1H), 1.43 (s, 3H), 1.41-1.31 (m, 6H).

| Compound | | |
|---|---|---|
| Number | Synthesis | LC/MS |
| 585 | Similar to Example 88 | m/z 665.4 [M + H]⁺ |
| 586 | Similar to Example 88 | m/z 613.1 [M + H]⁺ |
| 587 | Similar to Example 88 | m/z 577.4 [M + H]⁺ |
| 588 | Similar to Example 88 | m/z 541.4 [M + H]⁺ |
| 589 | Similar to Example 88 | m/z 629.4 [M + H]⁺ |
| 590 | Similar to Example 88 | m/z 627.4 [M + H]⁺ |

### Example 89

### (R)-8-methyl-N-(1-(3-methyl-1H-1,2,4-triazol-5-yl)-2-oxabicyclo[2.1.1]hexan-4-yl)-6-(3-methyl-3-phenylpyrrolidin-1-yl)-[1,2,4]triazolo[1,5-a]pyridine-2-carboxamide (Compound 591)

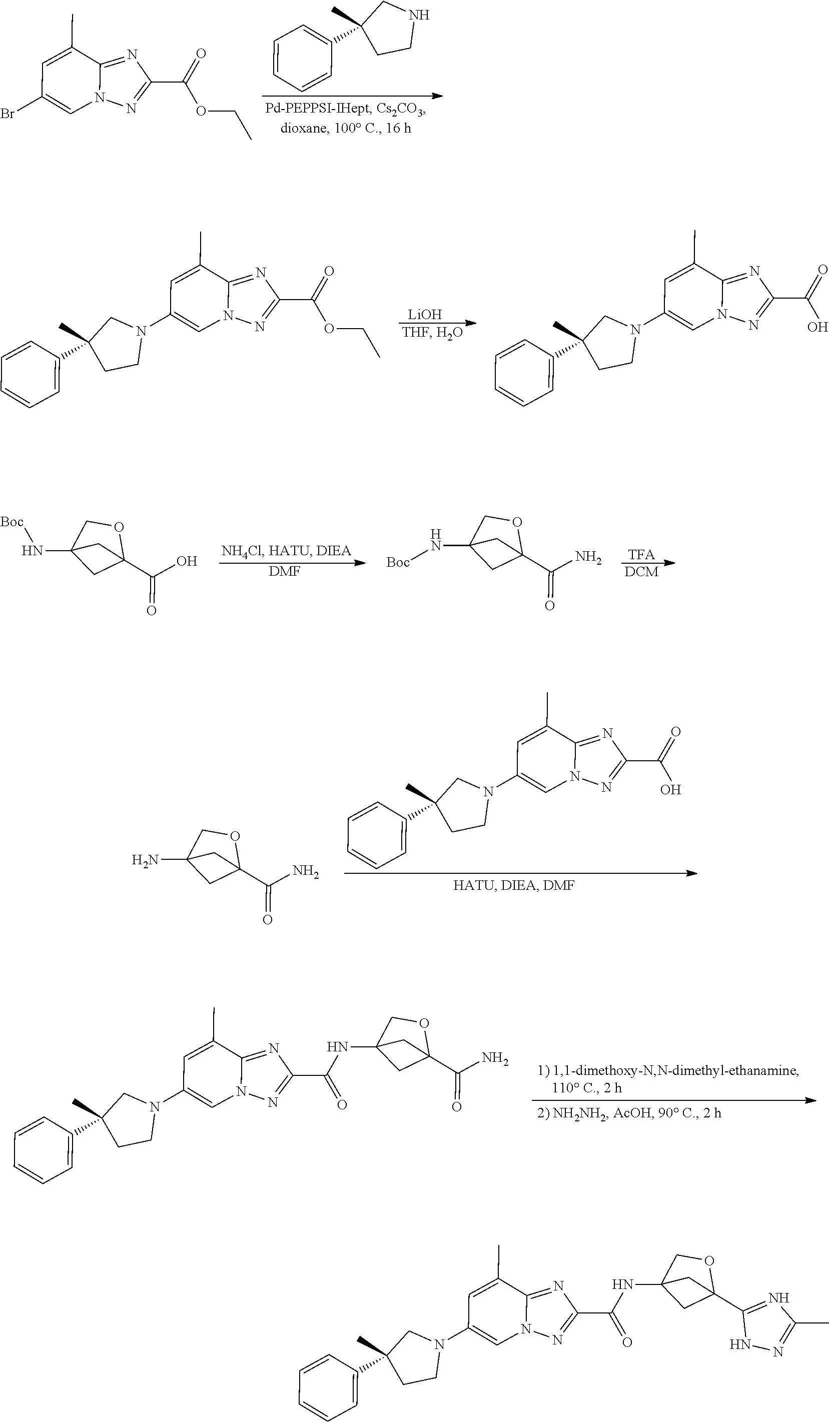

### Step 1: ethyl (R)-8-methyl-6-(3-methyl-3-phenylpyrrolidin-1-yl)-[1,2,4]triazolo[1,5-a]pyridine-2-carboxylate

To a solution of methyl 6-bromo-8-methyl-[1,2,4]triazolo[1,5-a]pyridine-2-carboxylate (1.4 g, 5.18 mmol, 1 eq) and (3R)-3-methyl-3-phenyl-pyrrolidine (835.82 mg, 5.18 mmol, 1 eq) in dioxane (50 mL) was added Cs₂CO₃ (5.07 g, 15.55 mmol, 3 eq) and 1,3-bis[2,6-bis(1-propylbutyl)phenyl]-4,5-dichloro-2H-imidazol-1-ium-2-ide;3-chloropyridine;dichloropalladium (151.27 mg, 155.51 μmol, 0.03 eq). The mixture was stirred at 100° C. for 12 h in N₂. LCMS showed starting material was consumed and one main peak with desired mass was detected. The reaction mixture was filtered and concentrated in vacuo. The residue was purified by flash silica gel chromatography (ISCO®; 20 g SepaFlash® Silica Flash Column, Eluent of 30~40% Ethyl acetate/Petroleum ethergradient @80 mL/min) to give methyl 8-methyl-6-[(3R)-3-methyl-3-phenyl-pyrrolidin-1-yl]-[1,2,4]triazolo[1,5-a]pyridine-2-carboxylate (1.3 g, 72% yield) as a yellow oil.

LC-MS [ESI, M+1]: 351.2

¹H NMR (400 MHz, CHLOROFORM-d) δ=7.63 (d, J=1.6 Hz, 1H), 7.35-7.29 (m, 2H), 7.29-7.24 (m, 2H), 7.24-7.18 (m, 1H), 6.98 (s, 1H), 4.04-3.99 (m, 3H), 3.56 (d, J=8.8 Hz, 1H), 3.52-3.42 (m, 2H), 3.40-3.33 (m, 1H), 2.67-2.61 (m, 3H), 2.34 (td, J=8.4, 12.0 Hz, 1H), 2.27-2.16 (m, 1H), 1.42 (s, 3H)

### Step 2: (R)-8-methyl-6-(3-methyl-3-phenylpyrrolidin-1-yl)-[1,2,4]triazolo[1,5-a]pyridine-2-carboxylic acid

To a solution of methyl (R)-8-methyl-6-(3-methyl-3-phenylpyrrolidin-1-yl)-[1,2,4]triazolo[1,5-a]pyridine-2-carboxylate (400 mg, 1.14 mmol, 1 eq) in THF (4 mL) and H₂O (0.5 mL) was added LiOH·H₂O (119.75 mg, 2.85 mmol, 2.5 eq). The mixture was stirred at 25° C. for 2 h. LCMS showed starting material was consumed and one main peak with desired mass was detected. The reaction mixture was added HCl (1 M) until pH around 4 and then extracted with EtOAc (10 mL×3). The combined organic phase was washed with brine (10 mL×3), dried over anhydrous Na₂SO₄, filtered and concentrated in vacuo. to give compound (R)-8-methyl-6-(3-methyl-3-phenylpyrrolidin-1-yl)-[1,2,4]triazolo[1,5-a]pyridine-2-carboxylic acid (350 mg, 1.04 mmol, 91% yield) as a white solid

LC-MS [ESI, M+1]: 377.3.

¹H NMR (400 MHz, CHLOROFORM-d) δ=7.87 (s, 1H), 7.41-7.35 (m, 2H), 7.35-7.31 (m, 2H), 7.25 (br s, 1H), 7.07 (s, 1H), 3.65 (d, J=9.2 Hz, 1H), 3.61-3.51 (m, 2H), 3.48-3.42 (m, 1H), 2.71 (s, 3H), 2.41 (td, J=8.4, 12.0 Hz, 1H), 2.33-2.24 (m, 1H), 1.48 (s, 3H)

### Step 3: tert-butyl (1-carbamoyl-2-oxabicyclo[2.1.1]hexan-4-yl)carbamate

To a solution of 4-(tert-butoxycarbonylamino)-2-oxabicyclo[2.1.1]hexane-1-carboxylic acid (200 mg, 822.18 μmol, 1 eq) in DMF (2 mL) was added DIEA (318.78 mg, 2.47 mmol, 429.63 μL, 3 eq), NH₄Cl (65.97 mg, 1.23 mmol, 1.5 eq) and HATU (375.14 mg, 986.62 μmol, 1.2 eq). The mixture was stirred at 25° C. for 2 h. LCMS showed starting material was consumed and the desired mass was detected. The mixture was quenched with water (10 mL) and extracted with ethyl acetate (15 mL×2). The combined organic phase was washed with brine (20 mL), dried with anhydrous Na₂SO₄, filtered and concentrated in vacuum to give compound tert-butyl N-(1-carbamoyl-2-oxabicyclo[2.1.1]hexan-4-yl)carbamate (250 mg, crude) was obtained as a yellow solid.

LC-MS [ESI, M-56]:187.1.

### Step 4: 4-amino-2-oxabicyclo[2.1.1]hexane-1-carboxamide

To a solution of tert-butyl N-(1-carbamoyl-2-oxabicyclo[2.1.1]hexan-4-yl)carbamate (200 mg, 825.52 μmol, 1 eq) in DCM (2 mL) was added TFA (1.54 g, 13.46 mmol, 1.00 mL, 16.31 eq). The mixture was stirred at 25° C. for 0.5 h. LCMS showed starting material was consumed and the desired mass was detected. The reaction mixture was concentrated under the reduced pressure to give compound 4-amino-2-oxabicyclo[2.1.1]hexane-1-carboxamide (200 mg, 780.71 μmol, 94.57% yield, TFA) was obtained as a yellow oil.

LC-MS [ESI, M+1]:143.2.

### Step 5: (R)—N-(1-carbamoyl-2-oxabicyclo[2.1.1]hexan-4-yl)-8-methyl-6-(3-methyl-3-phenylpyrrolidin-1-yl)-[1,2,4]triazolo[1,5-a]pyridine-2-carboxamide

To a solution of 8-methyl-6-[(3R)-3-methyl-3-phenyl-pyrrolidin-1-yl]-[1,2,4]triazolo[1,5-a]pyridine-2-carboxylic acid (50 mg, 148.64 μmol, 1 eq) in DMF (2 mL) was added 4-amino-2-oxabicyclo[2.1.1]hexane-1-carboxamide (57.12 mg, 222.96 μmol, 1.50 eq, TFA) HATU (67.82 mg, 178.37 μmol, 1.2 eq) and DIEA (57.63 mg, 445.92 μmol, 77.67 μL, 3 eq). The mixture was stirred at 25° C. for 1 h. LCMS showed starting material was consumed and the desired mass was detected. The reaction mixture was extracted with ethyl acetate, The aqueous phase was extracted with ethyl acetate (20 mL×2). The combined organic phase was washed with brine (30 mL), dried with anhydrous Na₂SO₄, filtered and concentrated in vacuum. The residue was purified by prep-HPLC (FA condition) to give compound N-(1-carbamoyl-2-oxabicyclo[2.1.1]hexan-4-yl)-8-methyl-6-[(3R)-3-methyl-3-phenyl-pyrrolidin-1-yl]-[1,2,4]triazolo[1,5-a]pyridine-2-carboxamide (40 mg, 73.83 μmol, 49.67% yield, 85% purity) as white solid.

LC-MS [ESI, M+1]:461.3.

¹H NMR (400 MHz, CHLOROFORM-d) δ=7.84 (br s, 1H), 7.66 (br s, 1H), 7.41-7.34 (m, 2H), 7.34-7.29 (m, 2H), 7.27-7.23 (m, 1H), 7.03 (br s, 1H), 6.47 (br s, 1H), 5.66 (br s, 1H), 4.18 (br s, 2H), 3.65-3.37 (m, 4H), 2.63 (s, 3H), 2.58 (br s, 2H), 2.44-2.22 (m, 4H), 1.47 (s, 3H)

### Step 6: (R)-8-methyl-N-(1-(3-methyl-1H-1,2,4-triazol-5-yl)-2-oxabicyclo[2.1.1]hexan-4-yl)-6-(3-methyl-3-phenylpyrrolidin-1-yl)-[1,2,4]triazolo[1,5-a]pyridine-2-carboxamide

To a solution of N-(1-carbamoyl-2-oxabicyclo[2.1.1]hexan-4-yl)-8-methyl-6-[(3R)-3-methyl-3-phenyl-pyrrolidin-1-yl]-[1,2,4]triazolo[1,5-a]pyridine-2-carboxamide (40 mg, 86.86 μmol, 1 eq) in 1,1-dimethoxy-N,N-dimethylethanamine (2.19 g, 16.42 mmol, 2.40 mL, 189.00 eq), the mixture was stirred at 110° C. for 2 h. Then the mixture was concentrated under reduced pressure to give a residue. To a solution of this residue in EtOH (0.6 mL) was added NH₂NH₂·H₂O (412.80 mg, 35.09 mmol, 400.00 μL, 85% purity, 403.97 eq) and AcOH (201.41 mg, 3.35 mmol, 192.00 μL, 38.61 eq) at −10° C. And then the mixture was stirred at 60° C. for 1 h. LCMS showed starting material was consumed and the desired mass was detected. The reaction mixture was extracted with ethyl acetate (10 mL×2). The combined organic phase was washed with brine (15 mL), dried with anhydrous Na₂SO₄, filtered and concentrated in vacuum. The residue was purified by prep-HPLC (FA condition column: Phenomenex luna C18 150*25 mm*10 um; mobile phase: [water(FA)—ACN]; gradient:32%-62% B over 10 min) to give compound 8-methyl-6-[(3R)-3-methyl-3-phenyl-pyrrolidin-1-yl]-N-[1-(3-methyl-1H-1,2,4-triazol-5-yl)-2-oxabicyclo[2.1.1]hexan-4-yl]-[1,2,4]triazolo[1,5-a]pyridine-2-carboxamide (2.15 mg, 4.19 μmol, 4.83% yield, 97.212% purity) as red solid.

LC-MS [ESI, M+1]:499.2.

¹H NMR (400 MHz, CHLOROFORM-d) δ=7.88 (s, 1H), 7.67 (s, 1H), 7.42-7.29 (m, 4H), 7.25 (br s, 1H), 7.02 (s, 1H), 4.25-4.11 (m, 2H), 3.61 (br d, J=8.8 Hz, 1H), 3.58-3.46 (m, 2H), 3.46-3.37 (m, 1H), 2.77-2.67 (m, 2H), 2.63 (s, 3H), 2.54-2.34 (m, 6H), 2.30-2.24 (m, 1H), 1.47 (s, 3H).

| Compound | | |
|---|---|---|
| Number | Synthesis | LC/MS |
| 592 | Similar to example 89 | m/z 474.2 [M + H]⁺ |
| 593 | Similar to example 89 | m/z 506.2 [M + H]⁺ |
| 594 | Similar to example 89 | m/z 490.3 [M + H]⁺ |
| 595 | Similar to example 89 | m/z 476.2 [M + H]⁺ |

### Example 90

### 5-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]tetrahydrofuran-2-carboxylic acid (Compound 596)

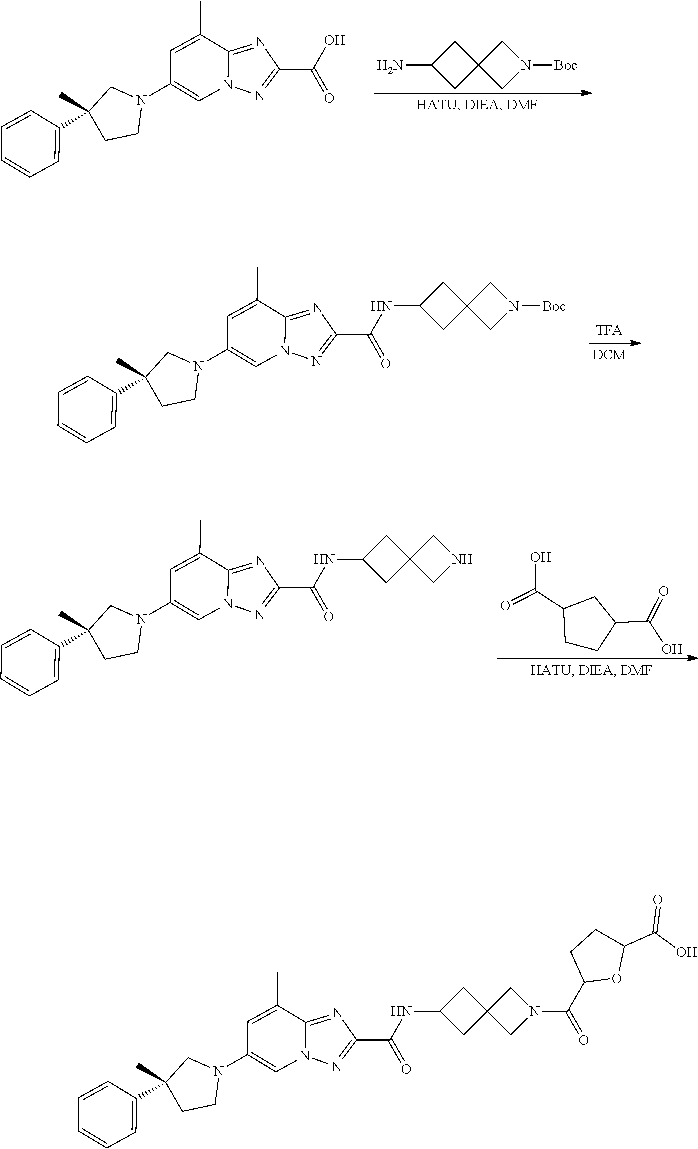 [SMILES: Cc1cc(N2CC[C@](C)(c3ccccc3)C2)cn2nc(C(=O)O)nc12 ; CC(C)(C)OC(=O)N1CC2(CC(N)C2)C1 ; Cc1cc(N2CC[C@](C)(c3ccccc3)C2)cn2nc(C(=O)NC3CC4(C3)CN(C(=O)OC(C)(C)C)C4)nc12 ; Cc1cc(N2CC[C@](C)(c3ccccc3)C2)cn2nc(C(=O)NC3CC4(CNC4)C3)nc12 ; O=C(O)C1CCC(C(=O)O)C1 ; Cc1cc(N2CC[C@](C)(c3ccccc3)C2)cn2nc(C(=O)NC3CC4(C3)CN(C(=O)C3CCC(C(=O)O)O3)C4)nc12]

### Step 1: tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylate

To a solution of 8-methyl-6-[(3R)-3-methyl-3-phenyl-pyrrolidin-1-yl]-[1,2,4]triazolo[1,5-a]pyridine-2-carboxylic acid (50 mg, 148.64 μmol, 1 eq) and tert-butyl 6-amino-2-azaspiro[3.3]heptane-2-carboxylate (34.71 mg, 163.50 μmol, 1.1 eq) in DMF (1 mL) was added DIEA (57.63 mg, 445.92 μmol, 77.67 μL, 3 eq) and HATU (67.82 mg, 178.37 μmol, 1.2 eq) and the reaction was stirred at 25° C. 1 h. LC-MS showed reactant was consumed completely and one main peak with desired mass was detected. The reaction was diluted with H₂O (3 mL) and extracted with EtOAc (5 mL×3). The combined organic layers were washed with brine (5 mL×2), dried over Na₂SO₄, filtered and the filtrate was concentrated under reduced pressure to give a residue. The residue was purified by prep-TLC (SiO₂, PE:EtOAc=0:1, Rf=0.3) to give tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylate (50 mg, 93.28 μmol, 62.76% yield, 99% purity) as yellow oil.

LC-MS [ESI, M+1]: 531.3

¹H NMR (400 MHz, DMSO-d₆) δ=8.70 (d, J=8.0 Hz, 1H), 7.87 (d, J=1.6 Hz, 1H), 7.42-7.29 (m, 5H), 7.27-7.21 (m, 1H), 4.32 (sxt, J=8.0 Hz, 1H), 3.91 (s, 2H), 3.77 (s, 2H), 3.63-3.57 (m, 1H), 3.55-3.45 (m, 2H), 3.39-3.36 (m, 1H), 2.69 (s, 3H), 2.49-2.42 (m, 2H), 2.41-2.32 (m, 2H), 2.26 (t, J=7.2 Hz, 2H), 1.43-1.32 (m, 12H).

### Step 2: N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide

To a solution of tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylate (50 mg, 94.22 μmol, 1 eq) in DCM (0.5 mL) was added TFA (153.50 mg, 1.35 mmol, 0.1 mL, 14.29 eq). The mixture was stirred at 25° C. for 1 h. LC-MS showed reactant was consumed completely and one main peak with desired mass was detected. The reaction was concentrated under reduced pressure to give N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide (40 mg, 92.91 μmol, 98.60% yield) as yellow oil.

LC-MS [ESI, M+1]: 431.2

### Step 3: 5-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]tetrahydrofuran-2-carboxylic acid

To a solution of N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide (20 mg, 46.45 μmol, 1 eq) and tetrahydrofuran-2,5-dicarboxylic acid (29.75 mg, 185.81 μmol, 4 eq) in DMF (0.6 mL) was added HATU (21.20 mg, 55.74 μmol, 1.2 eq) and DIEA (18.01 mg, 139.36 μmol, 24.27 μL, 3 eq). The mixture was stirred at 25° C. for 0.5 h. LC-MS showed reactant was consumed completely and one main peak with desired mass was detected. The reaction was diluted with H₂O (6 mL) and extracted with DCM (8 mL×3). The combined organic layers were washed with brine (8 mL×2), dried over Na₂SO₄, filtered and the filtrate was concentrated under reduced pressure to give a residue. The residue was purified by prep-HPLC (column: CD02-Waters Xbidge BEH C18 150×25×10 um; mobile phase: [water(NH₄HCO₃)—ACN]; gradient:18%-48% B over 10 min) to give 5-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]tetrahydrofuran-2-carboxylic acid (6.56 mg, 11.30 μmol, 24.32% yield, 98.6% purity) as a white solid.

LC-MS [ESI, M+1]: 573.2

¹H NMR (400 MHz, DMSO-d₆) δ=8.74 (dd, J=4.0, 8.0 Hz, 1H), 7.87 (s, 1H), 7.43-7.30 (m, 5H), 7.28-7.20 (m, 1H), 4.74-4.62 (m, 1H), 4.48-4.25 (m, 0.5H), 4.15 (d, J=9.6 Hz, 0.5H), 4.06 (s, 1H), 3.99-3.89 (m, 1H), 3.63-3.56 (m, 1H), 3.56-3.46 (m, 2H), 3.41-3.37 (m, 1H), 2.56 (s, 5H), 2.46-2.37 (m, 2H), 2.34-2.14 (m, 4H), 2.01-1.83 (m, 2H), 1.36 (s, 3H).

| Compound | | |
|---|---|---|
| Number | Synthesis | LC/MS |
| 597 | Similar to Example 90 | m/z 475.3 [M + H]⁺ |
| 598 | Similar to Example 90 | m/z 506.2 [M + H]⁺ |
| 599 | Similar to Example 90 | m/z 557.6 [M + H]⁺ |
| 600 | Similar to Example 90 | m/z 529.6 [M + H]⁺ |
| 601 | Similar to Example 90 | m/z 559.2 [M + H]⁺ |
| 602 | Similar to Example 90 | m/z 476.7 [M + H]⁺ |
| 603 | Similar to Example 90 | m/z 476.2 [M + H]⁺ |
| 604 | Similar to Example 90 | m/z 476.1 [M + H]⁺ |
| 605 | Similar to Example 90 | m/z 485.55 [M + H]⁺ |
| 606 | Similar to Example 90 | m/z 502.2 [M + H]⁺ |
| 607 | Similar to Example 90 | m/z 476.2 [M + H]⁺ |
| 597 | Similar to Example 90 | m/z 605.4 [M + H]⁺ |
| 592 | Similar to Example 90 | m/z 474.2 [M + H]⁺ |

### Example 91

### 8-methyl-6-[(3R)-3-methyl-3-phenyl-pyrrolidin-1-yl]-N-[1-[6-(methylsulfonimidoyl)-2-pyridyl]-4-piperidyl]-[1,2,4]triazolo[1,5-a]pyridine-2-carboxamide (Compound 608)

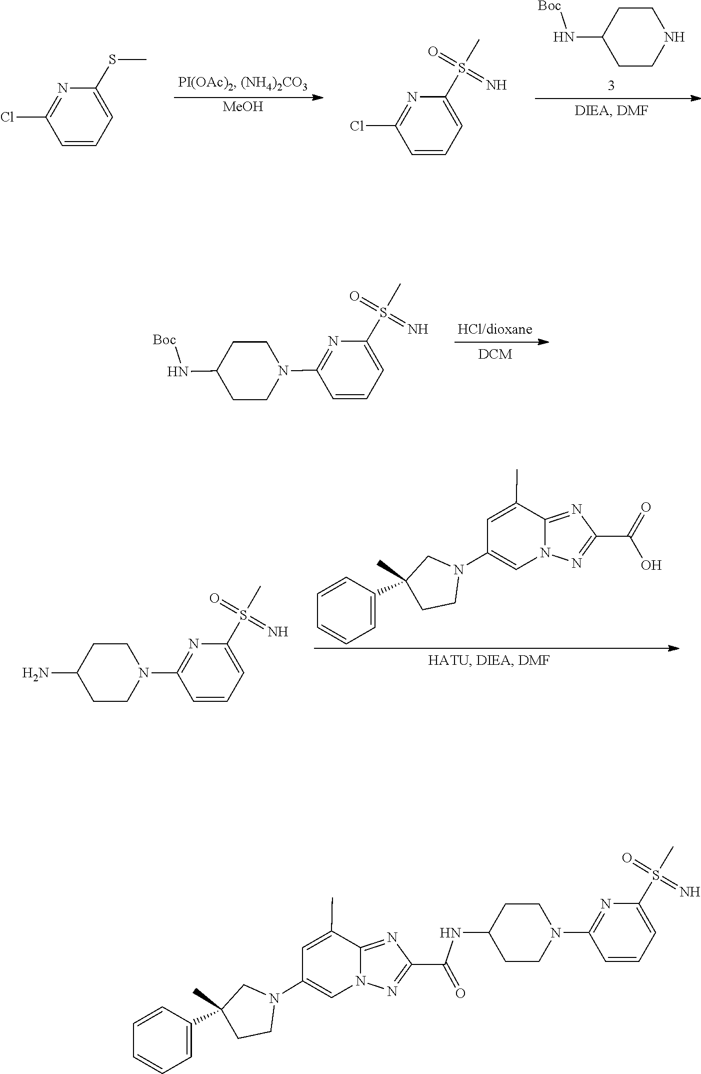

### Step 1: (6-chloro-2-pyridyl)-imino-methyl-oxo-sulfane

To a solution of 2-chloro-6-methylsulfanyl-pyridine (2 g, 12.53 mmol, 1 eq) in MeOH (20 mL) was added ammonia;carbonic acid (2.41 g, 25.06 mmol, 2.68 mL, 2 eq) and [acetoxy(phenyl)-iodanyl]acetate (10.09 g, 31.32 mmol, 2.5 eq). The mixture was stirred at 25° C. for 2 h. LCMS showed starting material was consumed and the desired mass was detected. The mixture was concentrated in vacuum and extracted with EtOAc (20 mL×3). The combined organic layer was washed with brine (20 mL), dried over sodium sulfate, concentrated in vacuum to afford crude. The residue was purified by flash silica gel chromatography (SiO₂, PE:EtOAc=3:1, Rf=0.3) to give compound (6-chloro-2-pyridyl)-imino-methyl-oxo-sulfane (1.3 g, 54.43% yield) as a yellow solid.

LC-MS [ESI, M+1]: 191.1

¹H NMR (400 MHz, CHLOROFORM-d) δ=8.03 (d, J=7.6 Hz, 1H), 7.90 (t, J=7.6 Hz, 1H), 7.52 (d, J=8.00 Hz, 1H), 3.26 (s, 3H).

### Step 2: tert-butyl N-[1-[6-(methylsulfonimidoyl)-2-pyridyl]-4-piperidyl]carbamate

To a solution of (6-chloro-2-pyridyl)-imino-methyl-oxo-sulfane (600 mg, 3.15 mmol, 1 eq) and tert-butyl N-(4-piperidyl)carbamate (630.30 mg, 3.15 mmol, 1 eq) in DMF (5 mL) was added DIEA (1.22 g, 9.44 mmol, 1.64 mL, 3 eq). The mixture was stirred at 100° C. for 2 h. LCMS showed starting material was consumed and the desired mass was detected. The mixture was concentrated in vacuum and extracted with EtOAc (3 mL×3). The combined organic layer was washed with brine (3 mL), dried over sodium sulfate, concentrated in vacuum to afford crude. The residue was purified by flash silica gel chromatography (SiO₂, PE:EtOAc=3:1, Rf=0.3) to give compound tert-butyl N-[1-[6-(methylsulfonimidoyl)-2-pyridyl]-4-piperidyl]carbamate (370 mg, 1.04 mmol, 33.17% yield) as a yellow solid.

LC-MS [ESI, M+1]:355.1

¹H NMR (400 MHz, CHLOROFORM-d) δ=7.63 (dd, J=8.4, 7.2 Hz, 1H). 7.32 (d, J=7.2 Hz, 1H) 6.81 (d, J=8.80 Hz, 1H) 4.71-4.37 (m, 1H) 4.36-4.21 (m, 2H) 3.81-3.60 (m, 1H) 3.30-3.17 (m, 3H) 3.10-3.01 (m, 2H) 2.87-2.62 (m, 1H) 2.07 (br s, 1H) 1.61 (br s, 2H) 1.46-1.36 (m, 9H)

### Step 3: 1-[6-(methylsulfonimidoyl)-2-pyridyl]piperidin-4-amine

To a solution of tert-butyl N-[1-[6-(methylsulfonimidoyl)-2-pyridyl]-4-piperidyl]carbamate (100 mg, 282.11 μmol, 1 eq) in DCM (2 mL) was added HCl/dioxane (2 mL). The mixture was stirred at 25° C. for 2 h. LCMS showed starting material was consumed and the desired mass was detected. The reaction was filtered and the filtered concentrated under reduced pressure to give compound 1-[6-(methylsulfonimidoyl)-2-pyridyl]piperidin-4-amine (70 mg, 85.32% yield, HCl) as a white solid.

LC-MS [ESI, M+1]:255.4

### Step 4: 8-methyl-6-[(3R)-3-methyl-3-phenyl-pyrrolidin-1-yl]-N-[1-[6-(methylsulfonimidoyl)-2-pyridyl]-4-piperidyl]-[1,2,4]triazolo[1,5-a]pyridine-2-carboxamide

To a solution of 1-[6-(methylsulfonimidoyl)-2-pyridyl]piperidin-4-amine (38.90 mg, 133.77 μmol, 1.5 eq, HCl) and 8-methyl-6-[(3R)-3-methyl-3-phenyl-pyrrolidin-1-yl]-[1,2,4]triazolo[1,5-a]pyridine-2-carboxylic acid (30 mg, 89.18 μmol, 1 eq) in DMF (2 mL) was added HATU (50.87 mg, 133.77 μmol, 1.5 eq) and DIEA (34.58 mg, 267.55 μmol, 46.60 μL, 3 eq). The mixture was stirred at 25° C. for 2 h. LCMS showed starting material was consumed and the desired mass was detected. The mixture was concentrated in vacuum and extracted with EtOAc (3 mL×3). The combined organic layer was washed with brine (3 mL), dried over sodium sulfate, concentrated in vacuum to afford crude. The residue was purified by prep-HPLC (column: Phenomenex luna C18 150*25 mm*10 um; mobile phase: [water(FA)—ACN]; gradient:32%-62% B over 15 min). The result solution was lyophilized to give compound 8-methyl-6-[(3R)-3-methyl-3-phenyl-pyrrolidin-1-yl]-N-[1-[6-(methylsulfonimidoyl)-2-pyridyl]-4-piperidyl]-[1,2,4]triazolo[1,5-a]pyridine-2-carboxamide (50.72 mg, 97.11% yield, 97.79% purity) as a white solid.

LC-MS [ESI, M+1]: 573.3

¹H NMR (400 MHz, DMSO-d₆) δ=8.42 (d, J=8.4 Hz, 1H). 7.88 (s, 1H) 7.75 (t, J=8.0 Hz, 1H) 7.42-7.29 (m, 5H) 7.26-7.19 (m, 2H) 7.09 (d, J=8.8 Hz, 1H) 4.39 (br d, J=13.2 Hz, 2H) 4.28-4.02 (m, 2H) 3.64-3.57 (m, 1H) 3.55-3.46 (m, 2H) 3.41-3.35 (m, 1H) 3.10 (s, 3H) 3.00 (br t, J=12.0 Hz, 2H) 2.55 (s, 3H) 2.31-2.20 (m, 2H) 1.87 (br d, J=10.0 Hz, 2H) 1.75-1.60 (m, 2H) 1.36 (s, 3H)

| Compound | | |
|---|---|---|
| Number | Synthesis | LC/MS |
| 608 | Example 91 | m/z 573.3 [M + H]⁺ |

### Example 92

### 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (Compound 609)

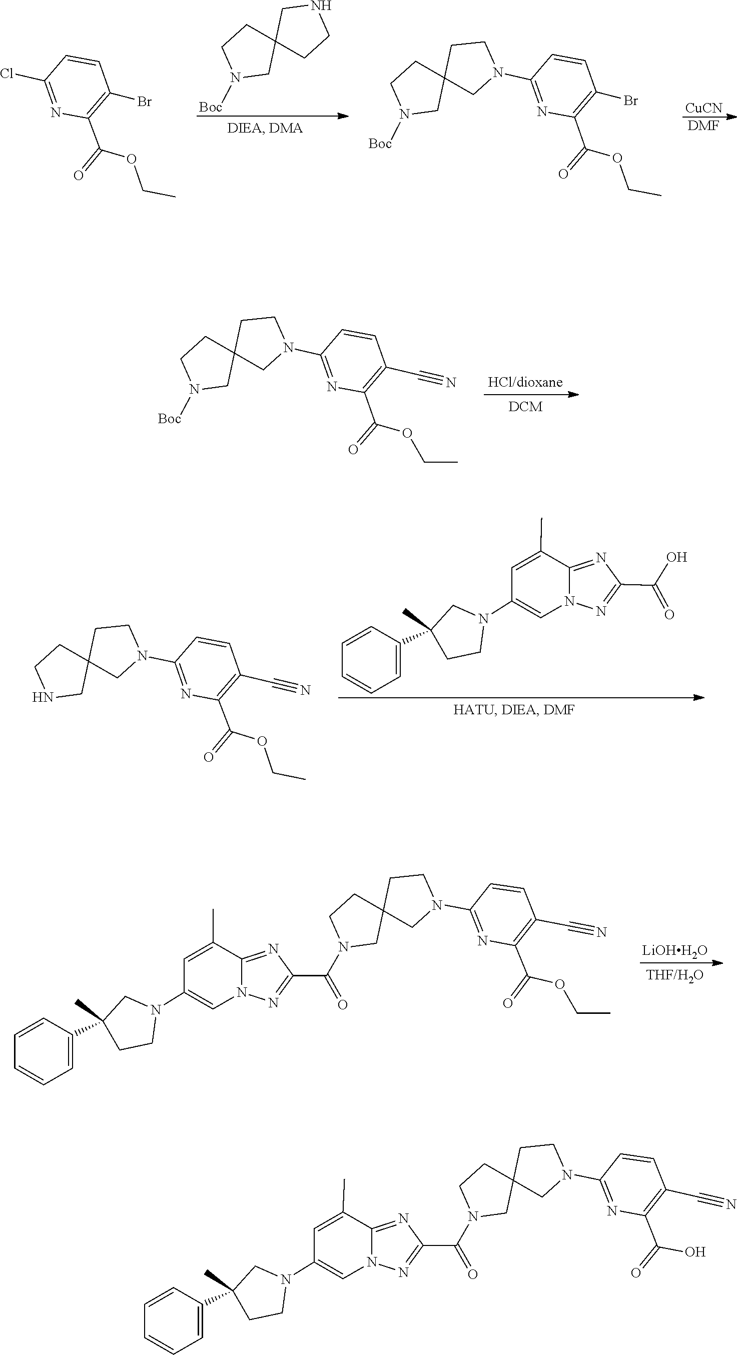

### Step 1: tert-butyl 7-(5-bromo-6-ethoxycarbonyl-2-pyridyl)-2,7-diazaspiro[4.4]nonane-2-carboxylate

To a solution of ethyl 3-bromo-6-chloro-pyridine-2-carboxylate (1 g, 3.78 mmol, 1 eq) and tert-butyl 2,7-diazaspiro[4.4]nonane-2-carboxylate (1.28 g, 5.67 mmol, 1.5 eq) in DMA (15 mL) was added DIEA (1.47 g, 11.34 mmol, 1.98 mL, 3 eq). The mixture was stirred at 100° C. for 1 h. LCMS showed reactant was consumed completely and the desired compound was detected. The reaction was added H₂O (10 mL) and then extracted with EtOAc (50 mL×3).The combined organic phase was washed with brine (20 mL×3), dried over anhydrous Na₂SO₄, filtered and concentrated in vacuum. The residue was purified by column chromatography (SiO₂, PE:EtOAc=2:1, Rf=0.5) to give compound tert-butyl 7-(5-bromo-6-ethoxycarbonyl-2-pyridyl)-2,7-diazaspiro[4.4]nonane-2-carboxylate (1.3 g, 76% yield) as a yellow oil.

LC-MS [ESI, M+1]: 454.1.

¹H NMR (400 MHz, CHLOROFOR-d) δ=7.58 (d, J=9.2 Hz, 1H), 6.31 (d, J=9.2 Hz, 1H), 4.44 (q, J=7.2 Hz, 2H), 3.56-3.41 (m, 8H), 2.07-1.82 (m, 4H), 1.50-1.39 (m, 12H).

### Step 2: tert-butyl 7-(5-cyano-6-ethoxycarbonyl-2-pyridyl)-2,7-diazaspiro[4.4]nonane-2-carboxylate

To a solution of tert-butyl 7-(5-bromo-6-ethoxycarbonyl-2-pyridyl)-2,7-diazaspiro[4.4]nonane-2-carboxylate (300 mg, 660.27 μmol, 1 eq) in DMF (4 mL) was added CuCN (118.27 mg, 1.32 mmol, 288.46 μL, 2 eq). The mixture was stirred at 100° C. for 1 h. LCMS showed reactant was not consumed completely and the desired compound was detected. The reaction mixture was filtered. The residue was purified by column chromatography (SiO₂, PE:EtOAc=1:1, Rf=0.5) and the eluted with EtOAc (50 mL) to give compound tert-butyl 7-(5-cyano-6-ethoxycarbonyl-2-pyridyl)-2,7-diazaspiro[4.4]nonane-2-carboxylate (100 mg, 37% yield) as a yellow oil.

LC-MS [ESI, M+1]: 401.2.

¹H NMR (400 MHz, CHLOROFORM-d) δ=7.71 (d, J=8.8 Hz, 1H), 6.48 (8 d, J=9.2 Hz, 1H), 4.49 (q, J=7.2 Hz, 2H), 3.82-3.21 (m, 8H), 2.14-1.98 (m, 2H), 1.97-1.84 (m, 2H), 1.50-1.42 (m, 12H).

### Step 3: ethyl 3-cyano-6-(2,7-diazaspiro[4.4]nonan-2-yl)pyridine-2-carboxylate

To a solution of tert-butyl 7-(5-cyano-6-ethoxycarbonyl-2-pyridyl)-2,7-diazaspiro[4.4]nonane-2-carboxylate (100 mg, 249.71 μmol, 1 eq) in DCM (1.5 mL) was added HCl/dioxane (2 M, 588.24 μL, 4.71 eq). The mixture was stirred at 25° C. for 0.5 h. LCMS showed reactant was consumed completely and the desired compound was detected. The reaction mixture was concentrated in vacuum to give compound ethyl 3-cyano-6-(2,7-diazaspiro[4.4]nonan-2-yl)pyridine-2-carboxylate (95 mg, crude, HCl) as a yellow oil.

LC-MS [ESI, M+1]: 301.2.

### Step 4: ethyl 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 8-methyl-6-[rac-(3R)-3-methyl-3-phenyl-pyrrolidin-1-yl]-[1,2,4]triazolo[1,5-a]pyridine-2-carboxylic acid (40 mg, 118.91 μmol, 1 eq) and ethyl 3-cyano-6-(2,7-diazaspiro[4.4]nonan-2-yl)pyridine-2-carboxylate (80.10 mg, 237.82 μmol, 2 eq, HCl) in DMF (1.5 mL) was added HATU (67.82 mg, 178.37 μmol, 1.5 eq) and DIEA (46.10 mg, 356.73 μmol, 62.14 μL, 3 eq). The mixture was stirred at 25° C. for 0.5 h. LCMS showed reactant was consumed completely and the desired compound was detected. The reaction was added H₂O (2 mL) and then extracted with EtOAc (15 mL×3).The combined organic phase was washed with brine (15 mL×3), dried over anhydrous Na₂SO₄, filtered and concentrated in vacuum. The residue was purified by column chromatography (SiO₂, PE:EtOAc=0:1, Rf=0.15) and the eluted with EtOAc (50 mL) to give compound ethyl 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (45 mg, 61% yield) as a yellow oil.

LC-MS [ESI, M+1]: 619.3.

### Step 5: 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of ethyl 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (40.00 mg, 64.65 μmol, 1 eq) in THF (1 mL) and H₂O (0.1 mL) was added LiOH·H₂O (5.43 mg, 129.30 μmol, 2 eq). The mixture was stirred at 25° C. for 0.5 h. LCMS showed reactant and the desired compound was detected. The reaction mixture was added FA until pH around 3 and the mixture was concentrated in vacuum. The residue was purified by prep-HPLC (column: Phenomenex luna C18 150*25 mm*10 um; mobile phase: [water(FA)—ACN]; gradient:43%-73% B over 10 min) to give compound 3-cyano-6-[2-[8-methyl-6-[rac-(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (21.94 mg, 57% yield) as a white solid.

LC-MS [ESI, M+1]: 591.2.

¹H NMR (400 MHz, CHLOROFORM-d) δ=7.78 (t, J=9.2 Hz, 1H), 7.65 (d, J=10.4 Hz, 1H), 7.43-7.27 (m, 5H), 7.04 (s, 1H), 6.65 (t, J=8.4 Hz, 1H), 4.23 (d, J=5.6 Hz, 1H), 4.06 (d, J=6.4 Hz, 1H), 3.97-3.81 (m, 2H), 3.76 (d, J=12.4 Hz, 1H), 3.71-3.34 (m, 7H), 2.64 (d, J=12.0 Hz, 3H), 2.44-2.34 (m, 1H), 2.27 (d, J=5.6 Hz, 1H), 2.11 (d, J=10.4 Hz, 4H), 1.47 (d, J=8.0 Hz, 3H).

| Compound | | |
|---|---|---|
| Number | Synthesis | LC/MS |
| 610 | Similar to Example 92 | m/z 609.5 [M + H]⁺ |
| 611 | Similar to Example 92 | m/z 552.3 [M + H]⁺ |
| 612 | Similar to Example 92 | m/z 556.3 [M + H]⁺ |
| 613 | Similar to Example 92 | m/z 542.2 [M + H]⁺ |
| 614 | Similar to Example 92 | m/z 584.3 [M + H]⁺ |
| 615 | Similar to Example 92 | m/z 538.3 [M + H]⁺ |
| 616 | Similar to Example 92 | m/z 526.3 [M + H]⁺ |
| 617 | Similar to Example 92 | m/z 554.4 [M + H]⁺ |
| 618 | Similar to Example 92 | m/z 568.2 [M + H]⁺ |
| 619 | Similar to Example 92 | m/z 596.3 [M + H]⁺ |
| 620 | Similar to Example 92 | m/z 566.3 [M + H]⁺ |
| 621 | Similar to Example 92 | m/z 552.2 [M + H]⁺ |
| 622 | Similar to Example 92 | m/z 538.2 [M + H]⁺ |
| 623 | Similar to Example 92 | m/z 566.2 [M + H]⁺ |
| 624 | Similar to Example 92 | m/z 566.3 [M + H]⁺ |
| 625 | Similar to Example 92 | m/z 596.3 [M + H]⁺ |
| 626 | Similar to Example 92 | m/z 567.3 [M + H]⁺ |
| 627 | Similar to Example 92 | m/z 580.3 [M + H]⁺ |
| 628 | Similar to Example 92 | m/z 538.4 [M + H]⁺ |
| 629 | Similar to Example 92 | m/z 552.7 [M + H]⁺ |
| 630 | Similar to Example 92 | m/z 582.3 [M + H]⁺ |
| 631 | Similar to Example 92 | m/z 552.3 [M + H]⁺ |
| 632 | Similar to Example 92 | m/z 584.2 [M + H]⁺ |

### Example 93

### 6-[rac-(5S)-8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid (Compound 633)

### 6-[rac-(5R)-8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid (Compound 634)

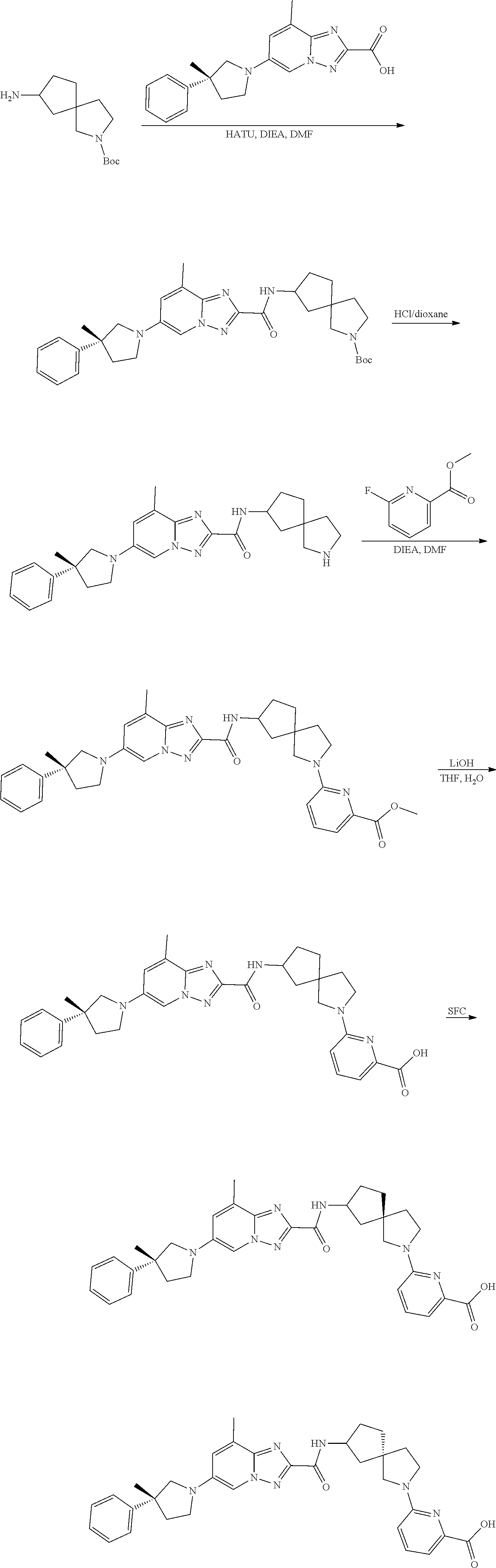

### Step 1: tert-butyl 8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonane-2-carboxylate

To a solution of 8-methyl-6-[(3R)-3-methyl-3-phenyl-pyrrolidin-1-yl]-[1,2,4]triazolo[1,5-a]pyridine-2-carboxylic acid (250 mg, 371.60 μmol, 1 eq) and tert-butyl 8-amino-2-azaspiro[4.4]nonane-2-carboxylate (98.24 mg, 408.76 μmol, 1.1 eq) in DMF (1 mL) was added HATU (211.94 mg, 557.39 μmol, 1.5 eq) and DIEA (144.08 mg, 1.11 mmol, 194.18 μL, 3 eq). The mixture was stirred at 25° C. for 0.5 h. LCMS showed starting material was consumed and the desired mass was detected. The reaction mixture was quenched by H₂O (5 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, hexanes:EtOAc=0:1, Rf=0.55) to give compound tert-butyl 8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonane-2-carboxylate (160 mg, 77% yield) as yellow gum.

LC-MS [ESI, M+1]: 559.6.

### Step 2: N-(2-azaspiro[4.4]nonan-8-yl)-8-methyl-6-[(3R)-3-methyl-3-phenyl-pyrrolidin-1-yl]-[1,2,4]triazolo[1,5-a]pyridine-2-carboxamide

To a solution of tert-butyl 8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonane-2-carboxylate (160 mg, 286.37 μmol, 1 eq) in DCM (2 mL) was added TFA (327 mg, 2.86 mmol, 0.221 mL, 10 eq) at 0° C. The mixture was stirred at 25° C. for 0.5 h. LCMS showed starting material was consumed and the desired mass was detected. The reaction mixture was filtered and the filtrate was concentrated under reduced pressure to give compound N-(2-azaspiro[4.4]nonan-8-yl)-8-methyl-6-[(3R)-3-methyl-3-phenyl-pyrrolidin-1-yl]-[1,2,4]triazolo[1,5-a]pyridine-2-carboxamide (160 mg, crude, TFA) as yellow gum.

LC-MS [ESI, M+1]: 459.2.

### Step 3: methyl 6-[8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylate

To a solution of N-(2-azaspiro[4.4]nonan-8-yl)-8-methyl-6-[(3R)-3-methyl-3-phenyl-pyrrolidin-1-yl]-[1,2,4]triazolo[1,5-a]pyridine-2-carboxamide (160 mg, 279.42 μmol, 1 eq, TFA) and methyl 6-fluoropyridine-2-carboxylate (130.03 mg, 838.25 μmol, 3 eq) in DMF (1 mL) was added DIEA (180.56 mg, 1.40 mmol, 243.35 μL, 5 eq). The mixture was stirred at 100° C. for 12 h. LCMS showed starting material was consumed and the desired mass was detected. The reaction mixture was quenched by H₂O (5 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.79) to give compound methyl 6-[8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylate (100 mg, 60% yield) as white solid.

LC-MS [ESI, M+1]: 594.3.

¹H NMR (400 MHz, CHLOROFORM-d) δ=7.68 (br s, 1H), 7.55 (br t, J=7.6 Hz, 1H), 7.41-7.29 (m, 6H), 7.03 (br d, J=4.4 Hz, 1H), 6.55 (d, J=8.4 Hz, 1H), 4.66 (br s, 1H), 4.00-3.93 (m, 3H), 3.77-3.33 (m, 8H), 2.42-2.19 (m, 4H), 2.07 (s, 3H), 2.03-1.92 (m, 2H), 1.88-1.72 (m, 4H), 1.49 (s, 3H).

### Step 4: 6-[8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid

To a solution of methyl 6-[8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylate (100 mg, 168.43 μmol, 1 eq) in THF (1 mL) was added LiOH·H₂O (14.14 mg, 336.86 μmol, 2 eq) and H₂O (0.3 mL). The mixture was stirred at 25° C. for 1 h. LCMS showed starting material was consumed and the desired mass was detected. The residue was purified by reversed-phase HPLC purification (mobile phase: [water(FA)—ACN]; B %: 33%-63%, 10 min), the mixture was concentrated and lyophilized to give compound 6-[8-[[8-methyl-6-[(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid (50.3 mg, 49% yield) as a white solid.

LC-MS [ESI, M+1]: 580.3.

¹H NMR (400 MHz, CHLOROFORM-d) δ=7.73-7.58 (m, 2H), 7.46 (br d, J=7.6 Hz, 1H), 7.40-7.31 (m, 5H), 7.01 (br s, 1H), 6.62 (br d, J=8.4 Hz, 1H), 4.67 (br d, J=7.6 Hz, 1H), 3.61 (br d, J=8.8 Hz, 1H), 3.59-3.49 (m, 5H), 3.49-3.38 (m, 2H), 2.64 (s, 3H), 2.41-2.33 (m, 2H), 2.28 (br d, J=7.2 Hz, 2H), 2.02 (br d, J=8.0 Hz, 2H), 1.88 (br s, 2H), 1.80 (br s, 2H), 1.47 (s, 3H).

### Step 5: 6-[rac-(5S)-8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid

The 6-[8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid was purified by prep-HPLC purification (mobile phase: [water(NH₄HCO₃)—ACN]; B %: 26%-56%, 10 min), the mixture was concentrated and lyophilized to give compound 6-[rac-(5S)-8-[[8-methyl-6-[rac-(3R)-3-methyl-3-phenyl-pyrrolidin-1-yl]-triazolo[1,5-a]pyridine-2-carbonyl]amino]-2-azaspiro[4.4]nonan-2-yl]pyridine-2-carboxylic acid (10.85 mg, 56% yield) as white solid.

LC-MS [ESI, M+1]: 580.4.

¹H NMR (400 MHz, CHLOROFORM-d) δ=7.67 (s, 1H), 7.63 (t, J=7.6 Hz, 1H), 7.44 (br d, J=7.2 Hz, 2H), 7.40-7.29 (m, 4H), 7.27 (s, 1H), 7.02 (s, 1H), 6.62 (d, J=8.4 Hz, 1H), 4.70-4.59 (m, 1H), 3.63-3.47 (m, 7H), 3.42 (br d, J=2.8 Hz, 1H), 2.64 (s, 3H), 2.36 (br d, J=9.6 Hz, 2H), 2.31-2.21 (m, 2H), 2.08-1.96 (m, 2H), 1.91 (br s, 1H), 1.78 (br dd, J=7.2, 12.8 Hz, 3H), 1.47 (s, 3H).

### Step 5: 6-[rac-(5R)-8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid

The 6-[8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid was purified by prep-HPLC purification (mobile phase: [water(NH₄HCO₃)—ACN]; B %: 26%-56%, 10 min), the mixture was concentrated and lyophilized to give compound 6-[rac-(5R)-8-[[8-methyl-6-[rac-(3R)-3-methyl-3-phenyl-pyrrolidin-1-yl]-[1,2,4]triazolo[1,5-a]pyridine-2-carbonyl]amino]-2-azaspiro[4.4]nonan-2-yl]pyridine-2-carboxylic acid (7.06 mg, 35% yield) as white solid.

LC-MS [ESI, M+1]: 580.4.

¹H NMR (400 MHz, CHLOROFORM-d) δ=7.65 (s, 1H), 7.62 (d, J=8.4 Hz, 1H), 7.45 (d, J=7.2 Hz, 1H), 7.40-7.34 (m, 3H), 7.34-7.29 (m, 2H), 7.27-7.23 (m, 1H), 7.01 (s, 1H), 6.60 (d, J=8.4 Hz, 1H), 4.72-4.55 (m, 1H), 3.67-3.48 (m, 5H), 3.45-3.34 (m, 3H), 2.63 (s, 3H), 2.47-2.33 (m, 2H), 2.32-2.20 (m, 2H), 2.15-1.99 (m, 2H), 1.93-1.84 (m, 1H), 1.83-1.65 (m, 3H), 1.47 (s, 3H).

| Compound | | |
|---|---|---|
| Number | Synthesis | LC/MS |
| 635 | Similar to | m/z 526.2 |
| | EXAMPLE 93 | [M + H]⁺ |

### Example 94

### 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylic acid (Compound 636)

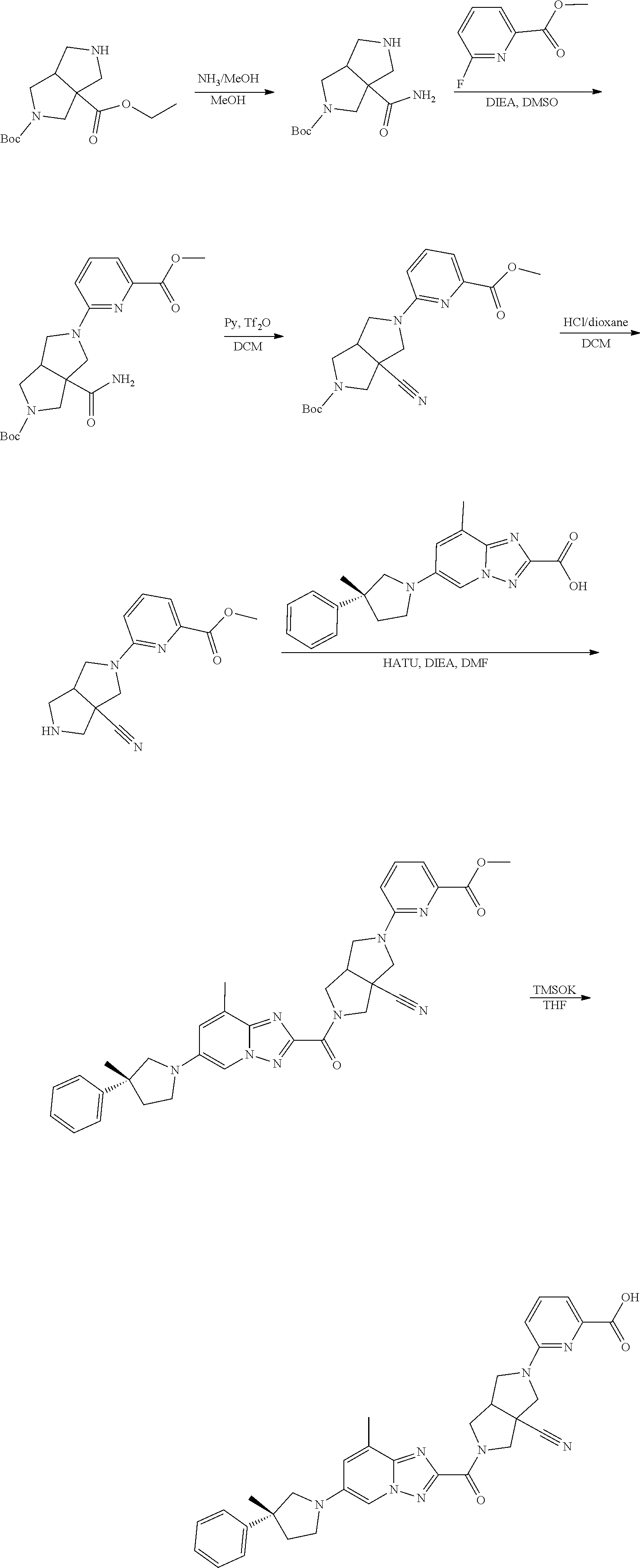

### Step 1: tert-butyl 3a-carbamoyl-1,2,3,4,6,6a-hexahydropyrrolo[3,4-c]pyrrole-5-carboxylate

To a solution of O5-tert-butyl O3a-ethyl 1,2,3,4,6,6a-hexahydropyrrolo[3,4-c]pyrrole-3a, 5-dicarboxylate (350 mg, 1.23 mmol, 1 eq) in MeOH (0.5 mL) was added NH₃/MeOH (7 M, 5 mL, 28.44 eq). The mixture was stirred at 60° C. for 0.5 h. LCMS showed starting material was consumed and one main peak with desired mass was detected. The reaction was concentrated under reduced pressure to give a residue. The residue was purified by prep-HPLC (neutral, column: Phenomenex luna C18 40-60 m, 40 g; mobile phase: [water(NH₄HCO₃)—ACN]; gradient:0%-15% B over 10 min) and lyophilized to give tert-butyl 3a-carbamoyl-1,2,3,4,6,6a-hexahydropyrrolo[3,4-c]pyrrole-5-carboxylate (300 mg, 95.46% yield) as a yellow solid.

LC-MS [ESI, M+1]: 256.1.

¹H NMR (400 MHz, ACETONITRILE-d3) 6 6.52 (s, 1H), 5.65 (s, 1H), 3.74 (d, J=11.6 Hz, 1H), 3.47 (dd, J=8.4, 11.2 Hz, 1H), 3.34 (d, J=11.6 Hz, 1H), 3.21 (dt, J=3.6, 11.2 Hz, 3H), 2.85 (d, J=3.6 Hz, 1H), 2.74 (d, J=10.8 Hz, 1H), 2.60 (dd, J=5.6, 10.4 Hz, 1H), 1.42 (s, 9H).

### Step 2: tert-butyl 3a-carbamoyl-2-(6-methoxycarbonyl-2-pyridyl)3,4,6,6atetrahydro-1H-pyrrolo[3,4-c]pyrrole-5-carboxylate

To a solution of tert-butyl 3a-carbamoyl-1,2,3,4,6,6a-hexahydropyrrolo[3,4-c]pyrrole-5-carboxylate (300 mg, 1.18 mmol, 1 eq) and methyl 6-fluoropyridine-2-carboxylate (218.73 mg, 1.41 mmol, 1.2 eq) in DMSO (4 mL) was added DIEA (759.30 mg, 5.88 mmol, 1.02 mL, 5 eq). The mixture was stirred at 100° C. for 1 h. LCMS showed starting material was consumed and one main peak with desired mass was detected. The mixture was quenched with H₂O (5 mL) and extracted with EtOAc (5 mL×3), the organic phase was dried, filtered, and concentrated under pressure to give the product. The crude product was triturated with PE (3 ml) and DCM (3 ml) at 25° C. for 10 min, filtered and the cake was dried in vacuum to give compound tert-butyl 3a-carbamoyl-2-(6-methoxycarbonyl-2-pyridyl)-3,4,6,6atetrahydro-1H-pyrrolo[3,4-c]pyrrole-5-carboxylate (320 mg, 819.60 μmol, 69.75% yield) as a yellow solid.

LC-MS [ESI, M+1]: 391.6.

¹H NMR (400 MHz, DMSO-d₆) δ 7.66 (t, J=8.0 Hz, 1H), 7.58 (s, 1H), 7.27 (d, J=7.6 Hz, 1H), 7.22 (s, 1H), 6.69 (d, J=8.4 Hz, 1H), 3.90-3.79 (m, 4H), 3.74-3.63 (m, 2H), 3.58 (d, J=10.4 Hz, 2H), 3.44 (d, J=11.2 Hz, 1H), 3.26 (d, J=5.2 Hz, 2H), 3.22-3.16 (m, 1H), 1.39 (s, 9H).

### Step 3: tert-butyl 3a-cyano-2-(6-methoxycarbonyl-2-pyridyl)-3,4,6,6a-tetrahydro-1H-pyrrolo[3,4-c]pyrrole-5-carboxylate

To a solution of tert-butyl 3a-carbamoyl-2-(6-methoxycarbonyl-2-pyridyl)-3,4,6,6a-tetrahydro-1H-pyrrolo[3,4-c]pyrrole-5-carboxylate (150 mg, 384.19 μmol, 1 eq) and Py (30.39 mg, 384.19 μmol, 31.01 μL, 1 eq) in DCM (3 mL) was added Tf₂O (108.40 mg, 384.19 μmol, 63.39 μL, 1 eq). The mixture was stirred at 0° C. for 0.5 h. LCMS showed starting material was consumed and one main peak with desired mass was detected. The mixture was adjusted to pH=9 with solid NaHCO₃. The residue was purified by prep-TLC (SiO₂, EtOAc:PE=1:1, Rf=0.7) to give compound tert-butyl 3a-cyano-2-(6-methoxycarbonyl-2-pyridyl)-3,4,6,6a-tetrahydro-1H-pyrrolo[3,4-c]pyrrole-5-carboxylate (140 mg, 95.89% yield) as a yellow gum.

LC-MS [ESI, M+1]: 373.5

### Step 4: methyl 6-(3a-cyano-1,2,3,4,6,6a-hexahydropyrrolo[3,4-c]pyrrol-5-yl)pyridine-2-carboxylate

To a solution of tert-butyl 3a-cyano-2-(6-methoxycarbonyl-2-pyridyl)-3,4,6,6a-tetrahydro-1H-pyrrolo[3,4-c]pyrrole-5-carboxylate (140 mg, 375.92 μmol, 1 eq) in DCM (2 mL) was added HCl/dioxane (2 M, 2 mL, 10.64 eq). The mixture was stirred at 25° C. for 0.5 h. LCMS showed starting material was consumed and the desired mass was detected. The reaction was concentrated under reduced pressure to give compound methyl 6-(3a-cyano-1,2,3,4,6,6a-hexahydropyrrolo[3,4-c]pyrrol-5-yl)pyridine-2-carboxylate (116 mg, crude, HCl) as a yellow gum.

LC-MS [ESI, M+1]: 273.1.

### Step 5: methyl 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (50 mg, 148.64 μmol, 1 eq) in DMF (1 mL) was added HATU (113.03 mg, 297.28 μmol, 2 eq) at 0° C. After addition, the mixture was stirred at this temperature for 0.5 h, and then methyl 6-(3a-cyano-1,2,3,4,6,6a-hexahydropyrrolo[3,4-c]pyrrol-5-yl)pyridine-2-carboxylate (55.07 mg, 178.37 μmol, 1.2 eq, HCl) and DIEA (96.05 mg, 743.19 μmol, 129.45 μL, 5 eq) was added at 0° C. The resulting mixture was stirred at 25° C. for 1 h. LCMS showed starting material was consumed and one main peak with desired mass was detected. The mixture was quenched with H₂O (1.5 mL) and extracted with EtOAc (2 mL×3), the organic phase was dried, filtered, and concentrated under pressure to give the product. The residue was purified by prep-TLC (SiO₂, EtOAc:NH₃·MeOH=10:1, Rf=0.75) to give compound methyl 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylate (25 mg, 28.47% yield) as a yellow gum.

LC-MS [ESI, M+1]: 591.7.

### Step 6: 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylic acid

To a solution of methyl 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylate (25 mg, 42.32 μmol, 1 eq) in THF (1 mL) was added TMSOK (8.14 mg, 63.49 μmol, 1.5 eq). The mixture was stirred at 0° C. for 0.5 h. LCMS showed starting material was consumed and one main peak with desired mass was detected. Acidify the clear solution with diluted hydrochloride acid. The reaction solution was filtered and the filtrate was and concentrated under pressure to give the residue. The residue was purified by prep-HPLC (column: CD04-Welch Utimate C18 150*25*7 um; mobile phase: [H₂O (FA)—ACN]; gradient:40%-70% B over 8 minhrhr) and lyophilized to give Compound 6-[3a-cyano-5-[8-methyl-6-[(3R)-3-methyl-3-phenyl-pyrrolidin-1-yl]-[1,2,4]triazolo[1,5-a]pyridine-2-carbonyl]-3,4,6,6a-tetrahydro-1H-pyrrolo[3,4-c]pyrrol-2-yl]pyridine-2-carboxylic acid (2.8 mg, 11.38% yield) as a white solid

LC-MS [ESI, M+1]: 577.5.

¹H NMR (400 MHz, CHLOROFORM-d) δ=7.79-7.71 (m, 1H), 7.65 (d, J=6.4 Hz, 1H), 7.61 (d, J=7.2 Hz, 1H), 7.40-7.34 (m, 2H), 7.34-7.30 (m, 2H), 7.25 (s, 1H), 7.02 (s, 1H), 6.68 (dd, J=5.6, 8.4 Hz, 1H), 4.75-4.58 (m, 1.5H), 4.40-4.20 (m, 2H), 4.18 (d, J=11.2 Hz, 1H), 4.07-3.99 (m, 1H), 3.97-3.88 (m, 1.5H), 3.68-3.59 (m, 2H), 3.58-3.46 (m, 3H), 3.46-3.38 (m, 1H), 2.64 (d, J=5.2 Hz, 3H), 2.44-2.35 (m, 1H), 2.32-2.23 (m, 1H), 1.47 (s, 3H).

### Example 95

### 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylic acid (Compound 637)

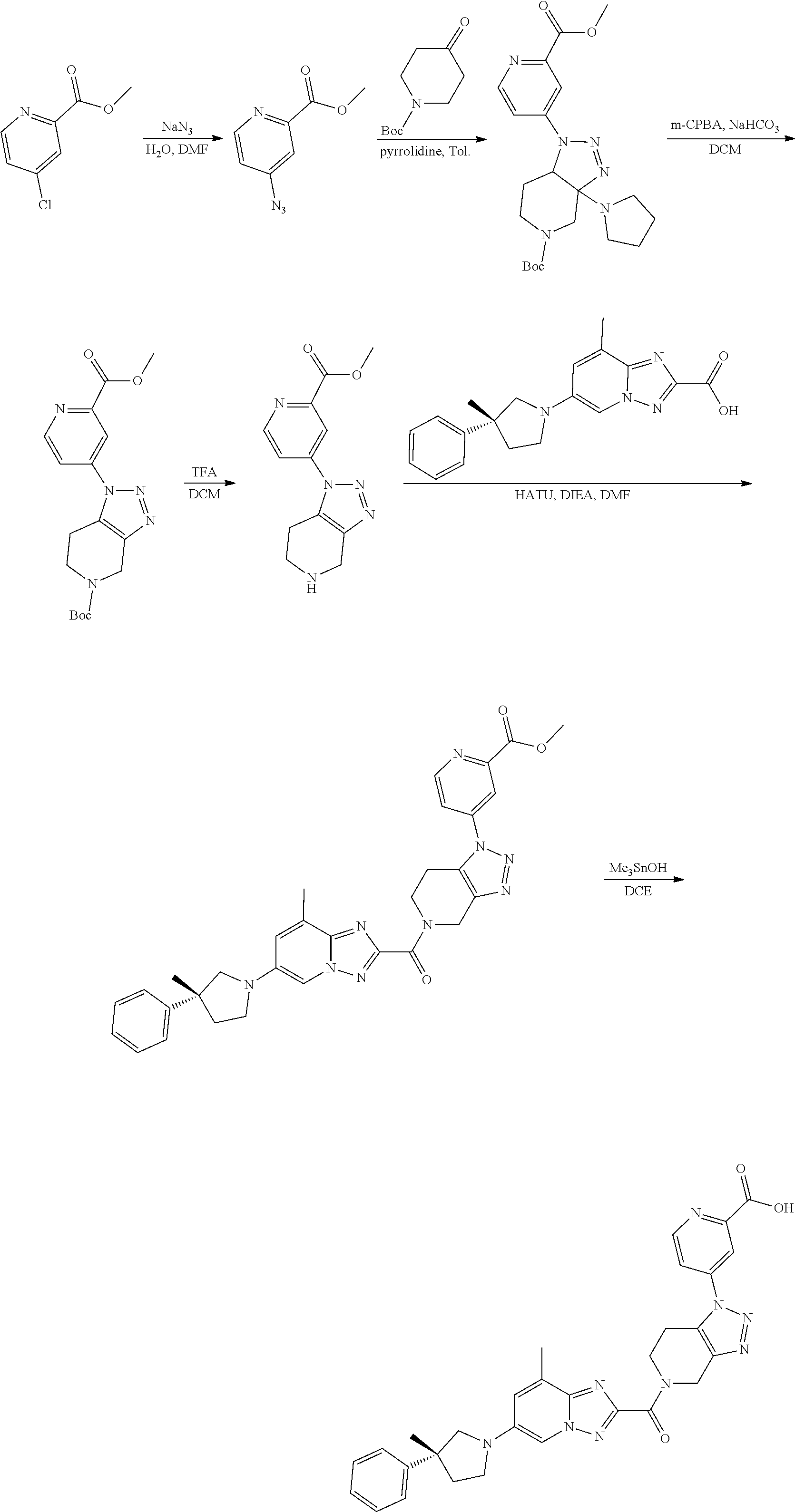

### Step 1: methyl 4-azidopyridine-2-carboxylate

To a solution of methyl 4-chloropyridine-2-carboxylate (1 g, 5.83 mmol, 1 eq) in DMF (10 mL) and H₂O (0.5 mL) was added NaN₃ (568.33 mg, 8.74 mmol, 1.5 eq). The mixture was stirred at 80° C. for 24 h. LCMS showed starting material consumed. Several new peaks were shown on LCMS and 95% of desired compound was detected. The reaction mixture was adjusted pH to 10 with Na₂CO₃ (10 mL) and then diluted with H₂O (20 mL) and extracted with EtOAc (30 mL×3). The combined organic layers were washed with brine (20 mL×3), dried over Na₂SO₄, filtered and concentrated under reduced pressure to give a crude methyl 4-azidopyridine-2-carboxylate (1 g, 91.50% yield) as yellow oil.

LC-MS [ESI, M-28+1]: 151.0.

### Step 2: tert-butyl 1-(2-methoxycarbonyl-4-pyridyl)-3a-pyrrolidin-1-yl-4,6,7,7a-tetrahydrotriazolo[4,5-c]pyridine5-carboxylate

To a solution of tert-butyl 4-oxopiperidine-1-carboxylate (430.17 mg, 2.16 mmol, 1 eq) in toluene (10 mL) was added methyl 4-azidopyridine-2-carboxylate (500 mg, 2.81 mmol, 1.3 eq) and pyrrolidine (153.55 mg, 2.16 mmol, 180.22 μL, 1 eq). The mixture was stirred at 100° C. for 3 h. LCMS showed starting material consumed. Several new peaks were shown on LCMS and 39% of desired compound was detected. Then the mixture was added NaHCO₃ (10 mL) and diluted with H₂O (20 mL) and extracted with EtOAc (30 mL×3). The combined organic layers were washed with brine (10 mL×3), dried over Na₂SO₄, filtered and concentrated under reduced pressure to give a residue. The residue was purified by flash silica gel chromatography (SiO₂, PE:EtOAc=0:1, Rf=0.3) to give compound tert-butyl 1-(2-methoxycarbonyl-4-pyridyl)-3a-pyrrolidin-1-yl-4,6,7,7a-tetrahydrotriazolo[4,5-c]pyridine5-carboxylate (615 mg, 66.17% yield) as yellow solid.

LC-MS [ESI, M-28+1]: 403.2.

¹H NMR (400 MHz, CHLOROFORM-d) δ=8.57 (br d, J=4.6 Hz, 1H), 8.38 (br d, J=7.2 Hz, 1H), 7.75 (br dd, J=5.6, 8.8 Hz, 1H), 4.78-4.40 (m, 2H), 4.01 (s, 3H), 3.43-3.28 (m, 2H), 2.87-2.77 (m, 1H), 2.62 (br d, J=6.4 Hz, 2H), 2.51-2.37 (m, 4H), 1.82-1.75 (m, 4H), 1.46-1.37 (m, 9H).

### Step 3: tert-butyl 1-(2-methoxycarbonyl-4-pyridyl)-6,7-dihydro-4H-triazolo[4,5-c]pyridine-5-carboxylate

To a solution of tert-butyl 1-(2-methoxycarbonyl-4-pyridyl)-3a-pyrrolidin-1-yl-4,6,7,7a-tetrahydrotriazolo[4,5-c]pyridine-5-carboxylate (430 mg, 998.84 μmol, 1 eq) in DCM (12 mL) was added NaHCO₃ (167.82 mg, 2.00 mmol, 77.73 μL, 2 eq) and m-CPBA (410.39 mg, 2.00 mmol, 84% purity, 2 eq) at 0° C. under N₂ atmosphere. The mixture was stirred at 25° C. for 0.5 h. LCMS showed starting material was consumed. Several new peaks were shown on LCMS and 45% of desired compound was detected. The reaction mixture was quenched with Na₂S203 (5 mL) and then diluted with H₂O (20 mL) and extracted with DCM (20 mL×3). The combined organic layers were washed with brine (10 mL×3), dried over Na₂SO₄, filtered and concentrated under reduced pressure to give a residue. The residue was purified by flash silica gel chromatography (SiO₂, PE:EtOAc=0:1, Rf=0.3) to give compound tert-butyl 1-(2-methoxycarbonyl-4-pyridyl)-6,7-dihydro-4H-triazolo[4,5-c]pyridine-5-carboxylate (240 mg, 64.75% yield) as white solid.

LC-MS [ESI, M-28+1]: 332.0.

¹H NMR (400 MHz, CHLOROFORM-d) δ=8.92 (d, J=5.2 Hz, 1H), 8.38 (s, 1H), 7.88 (br s, 1H), 4.74 (s, 2H), 4.06 (s, 3H), 3.81 (br s, 2H), 3.05 (br t, J=5.2 Hz, 2H), 1.51 (s, 9H).

### Step 4: methyl 4-(4,5,6,7-tetrahydrotriazolo[4,5-c]pyridin-1-yl)pyridine-2-carboxylate

To a solution of tert-butyl 1-(2-methoxycarbonyl-4-pyridyl)-6,7-dihydro-4H-triazolo[4,5-c]pyridine-5-carboxylate (50 mg, 139.13 μmol, 1 eq) in DCM (0.5 mL) was added TFA (12 M, 0.3 mL, 25.88 eq). The mixture was stirred at 25° C. for 20 min. LCMS showed starting material was consumed. One main peak was shown on LCMS and 98% of desired compound was detected. The reaction mixture was concentrated under reduced pressure to give a crude methyl 4-(4,5,6,7-tetrahydrotriazolo[4,5-c]pyridin-1-yl)pyridine-2-carboxylate (50 mg, 94.50% yield, TFA) as yellow oil.

LC-MS [ESI, M-28+1]: 232.1.

### Step 5: methyl 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (45 mg, 133.77 μmol, 1 eq) in DMF (0.5 mL) was added dropwise methyl 4-(4,5,6,7-tetrahydrotriazolo[4,5-c]pyridin1-yl)pyridine-2-carboxylate (49.94 mg, 133.77 μmol, 1 eq, TFA), and HATU (76.30 mg, 200.66 μmol, 1.5 eq). After addition, the mixture was stirred at this temperature for 10 min, and then DIEA (69.16 mg, 535.10 μmol, 93.20 μL, 4 eq) was added. The resulting mixture was stirred at 25° C. for 20 min. LCMS showed starting material was consumed. Several new peaks were shown on LCMS and 62% of desired compound was detected. The reaction mixture was diluted with H₂O (5 mL) and extracted with EtOAc (10 mL×3). The combined organic layers were washed with brine (10 mL×3), dried over Na₂SO₄, filtered and concentrated under reduced pressure to give a residue. The residue was purified by prep-HPLC (FA condition; column: CD01-Phenomenex luna C18 150×25×10 um; mobile phase: [water(FA)—ACN]; gradient: 40%-70% B over 10 min) to give compound methyl 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylate (35 mg, 45.29% yield) as white solid.

LC-MS [ESI, M+1]: 578.1.

### Step 6: 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylic acid

To a solution of methyl 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylate (35 mg, 60.59 μmol, 1 eq) in DCE (0.5 mL) was added hydroxy(trimethyl)stannane (109.56 mg, 605.92 μmol, 10 eq). The mixture was stirred at 50° C. for 2 h. LCMS showed starting material was consumed. One main peak was shown on LCMS and 86% of desired compound was detected. The reaction mixture was quenched with KF aqueous (0.5 mL) and then was concentrated under reduced pressure to give a residue. The residue was purified by prep-HPLC (FA condition; column: CD01-Phenomenex luna C18 150×25×10 um; mobile phase: [water(FA)—ACN]; gradient: 35%-65% B over 10 min) to give compound 4-[5-[8-methyl-6-[(3R)-3-methyl-3-phenyl-pyrrolidin-1-yl]-[1,2,4]triazolo[1,5-a]pyridine-2-carbonyl]-6,7-dihydro-4H-triazolo[4,5-c]pyridin-1-yl]pyridine-2-carboxylic acid (4.14 mg, 12.05% yield) as white solid.

LC-MS [ESI, M+1]: 564.2.

¹H NMR (400 MHz, CHLOROFORM-d) δ=8.84 (d, J=5.6 Hz, 1H), 8.55-8.35 (m, 1H), 8.22 (br d, J=4.4 Hz, 1H), 7.99 (br d, J=4.4 Hz, 1H), 7.69 (br d, J=18.4 Hz, 1H), 7.36 (quin, J=7.6 Hz, 4H), 7.25 (br s, 1H), 7.04 (br s, 1H), 5.37 (s, 1H), 5.12 (br s, 1H), 4.35-4.16 (m, 2H), 3.64 (br dd, J=4.8, 8.7 Hz, 1H), 3.60-3.49 (m, 2H), 3.48-3.40 (m, 1H), 3.34 (br s, 1H), 3.25 (br s, 1H), 2.67 (br d, J=6.4 Hz, 3H), 2.44-2.37 (m, 1H), 2.32-2.26 (m, 1H), 1.48 (s, 3H).

| Compound | | |
|---|---|---|
| Number | Synthesis | LC/MS |
| 638 | Similar to | m/z 555.3 |
| | Example 95 | [M + H]⁺ |
| 639 | Similar to | m/z 563.62 |
| | Example 95 | [M + H]⁺ |

### Example 96

### 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid (Compound 640)

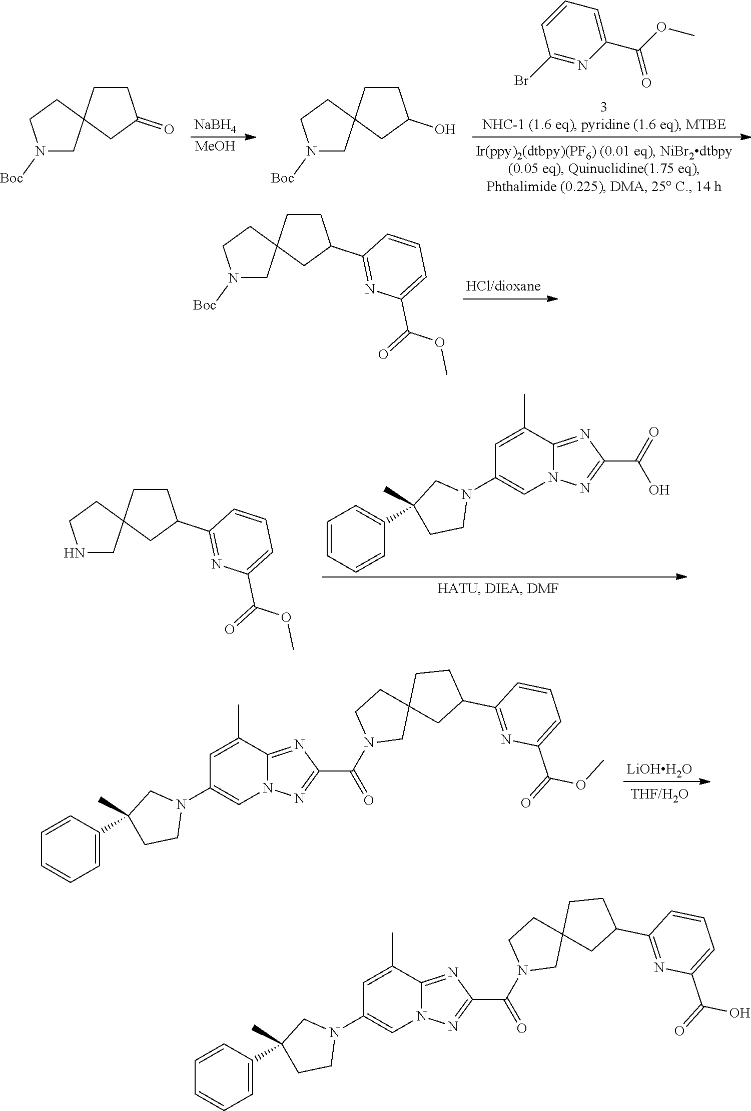

### Step 1: tert-butyl 8-hydroxy-2-azaspiro[4.4]nonane-2-carboxylate

To a solution of tert-butyl 8-oxo-2-azaspiro[4.4]nonane-2-carboxylate (150 mg, 626.80 μmol, 1 eq) in MeOH (2 mL) was added NaBH₄ (47.43 mg, 1.25 mmol, 2 eq) at 0° C. The mixture was stirred at 25° C. for 1 h. TLC indicated no starting material was remained and one major new spot with larger polarity was detected. The mixture was quenched with water (2 mL) and extracted with ethyl acetate (2 mL×3), the combined organic phase was dried over anhydrous sodium sulfate, filtered and concentrated to give tert-butyl 8-hydroxy-2-azaspiro[4.4]nonane-2-carboxylate (120 mg, 497.25 μmol, 79.3% yield) as colorless oil.

¹H NMR (400 MHz, CHLOROFORM-d) δ=4.40 (br s, 1H), 3.46-3.26 (m, 3H), 3.24-3.06 (m, 1H), 2.00-1.84 (m, 3H), 1.77-1.65 (m, 3H), 1.64-1.59 (m, 2H), 1.47 (s, 9H).

### Step 2: tert-butyl 8-(6-methoxycarbonyl-2-pyridyl)-2-azaspiro[4.4]nonane-2-carboxylate

To a solution of tert-butyl 8-hydroxy-2-azaspiro[4.4]nonane-2-carboxylate (117.29 mg, 486.04 μmol, 1.75 eq), NHC-1 (175.64 mg, 444.38 μmol, 1.6 eq) in MTBE (1.5 mL) was stirred at 25° C. for 5 min. To an 15 mL vial equipped with a stir bar was added pyridine (35.15 mg, 444.38 μmol, 35.87 μL, 1.6 eq), Ir(ppy)₂(dtbpy)(PF₆) (3.81 mg, 4.17 μmol, 0.015 eq), NiBr₂-dtbpy (10.14 mg, 20.83 μmol, 0.075 eq), quinuclidine (54.04 mg, 486.04 μmol, 1.75 eq), phthalamide (10.26 mg, 62.49 μmol, 0.225 eq), methyl 6-bromopyridine-2-carboxylate (60 mg, 277.74 μmol, 1 eq), DMA (1.5 mL) and above MTBE liquid. The vial was sealed and placed under nitrogen. The reaction was stirred and irradiated with a 10 W blue LED lamp (3 cm away), with cooling water to keep the reaction temperature at 25° C. for 14 h. LCMS showed starting material was consumed and desired mass was detected. he mixture was quenched with water (2 mL) and extracted with dichloromethane (2 mL×3), the combined organic phase was dried over anhydrous sodium sulfate, filtered and concentrated to give a residue. The residue was purified by prep-TLC (PE:EtOAc=1:1, Rf=0.5) to give tert-butyl 8-(6-methoxycarbonyl-2-pyridyl)-2-azaspiro[4.4]nonane-2-carboxylate (35 mg, 97.10 μmol, 34.9% yield) as yellow oil.

LC-MS [ESI, M+1]: 361.0.

¹H NMR (400 MHz, CHLOROFORM-d) δ=7.89 (dd, J=4.4, 7.2 Hz, 1H), 7.74-7.68 (m, 1H), 7.33 (br t, J=7.2 Hz, 1H), 3.93 (d, J=2.4 Hz, 3H), 3.56-3.13 (m, 5H), 2.26-2.04 (m, 2H), 1.90-1.66 (m, 6H), 1.40 (d, J=2.8 Hz, 9H).

### Step 3: methyl 6-(2-azaspiro[4.4]nonan-8-yl)pyridine-2-carboxylate

To a solution of tert-butyl 8-(6-methoxycarbonyl-2-pyridyl)-2-azaspiro[4.4]nonane-2-carboxylate (30 mg, 83.23 μmol, 1 eq) in DCM (0.5 mL) was added TFA (189.80 mg, 1.66 mmol, 123.65 μL, 20 eq). The mixture was stirred at 25° C. for 1 h. LCMS showed starting material was consumed and one peak with desired mass was detected. The residue was concentrated to give methyl 6-(2-azaspiro[4.4]nonan-8-yl)pyridine-2-carboxylate (31.16 mg, crude, TFA) as yellow oil.

LC-MS [ESI, M+1]: 261.1.

### Step 4: methyl 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (28 mg, 83.24 μmol, 1 eq), methyl 6-(2-azaspiro[4.4]nonan-8-yl)pyridine-2-carboxylate (31.16 mg, 83.24 μmol, 1 eq, TFA) in DMF (1 mL) was added HATU (47.47 mg, 124.86 μmol, 1.5 eq), DIEA (53.79 mg, 416.19 μmol, 72.49 μL, 5 eq). The mixture was stirred at 25° C. for 1 h. LCMS showed starting material was consumed and 68% of desired mass was detected. The mixture was quenched with water (2 mL) and extracted dichloromethane (2 mL×3), the combined organic phase was dried over anhydrous sodium sulfate, filtered and concentrated to give a residue. The residue was purified by prep-TLC (SiO₂, DCM:MeOH=10:1, Rf=0.22) to give methyl 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate (30 mg, 51.84 μmol, 62.28% yield) as white solid.

LC-MS [ESI, M+1]: 579.7.

¹H NMR (400 MHz, CHLOROFORM-d) δ=8.02-7.90 (m, 1H), 7.86-7.61 (m, 2H), 7.51-7.27 (m, 6H), 7.04 (br d, J=10.4 Hz, 1H), 4.14-3.90 (m, 5H), 3.85-3.66 (m, 2H), 3.66-3.39 (m, 5H), 2.67 (d, J=9.6 Hz, 3H), 2.45-2.33 (m, 1H), 2.33-2.16 (m, 3H), 2.04 (br d, J=5.6 Hz, 1H), 2.01-1.85 (m, 5H), 1.51-1.44 (m, 3H).

### Step 5: 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid

To a solution of methyl 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate (30 mg, 51.84 μmol, 1 eq) in THF (0.5 mL), H₂O (0.1 mL) was added LiOH·H₂O (6.53 mg, 155.52 μmol, 3 eq). The mixture was stirred at 25° C. for 1 h. LCMS showed starting material was consumed and 100% of desired mass was detected. The residue was added FA to adjust pH=4 and purified by prep-HPLC (column: CD01-Phenomenex luna C18 150*25*10 um; mobile phase: [water(FA)—ACN]; gradient:40%-70% B over 10 min) to 6-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid (16.47 mg, 29.05 μmol, 56% yield, 99.591% purity) as yellow solid.

LC-MS [ESI, M+1]:565.7.

¹H NMR (400 MHz, CHLOROFORM-d) δ=8.05 (br t, J=8.4 Hz, 1H), 7.91-7.80 (m, 1H), 7.75-7.61 (m, 1H), 7.52-7.40 (m, 1H), 7.40-7.28 (m, 4H), 7.27-7.21 (m, 1H), 6.99 (br s, 1H), 4.18-4.04 (m, 1H), 4.04-3.71 (m, 3H), 3.71-3.45 (m, 4H), 3.45-3.36 (m, 1H), 2.71-2.58 (m, 3H), 2.43-2.32 (m, 1H), 2.31-2.14 (m, 3H), 2.10-1.68 (m, 6H), 1.46 (s, 3H).

| | | |
|---|---|---|
| 641 | Similar to | m/z 580.4 |
| | Example 96 | [M + H]⁺ |

### Example 97

### 6-[2-[[8-methyl-6-[(3R)-3-methyl-3-phenyl-pyrrolidin-1-yl]-[1,2,4]triazolo[1,5-a]pyridine-2-carbonyl]amino]-7-azaspiro[3.5]nonan-7-yl]pyridine-2-carboxylic acid (Compound 642)

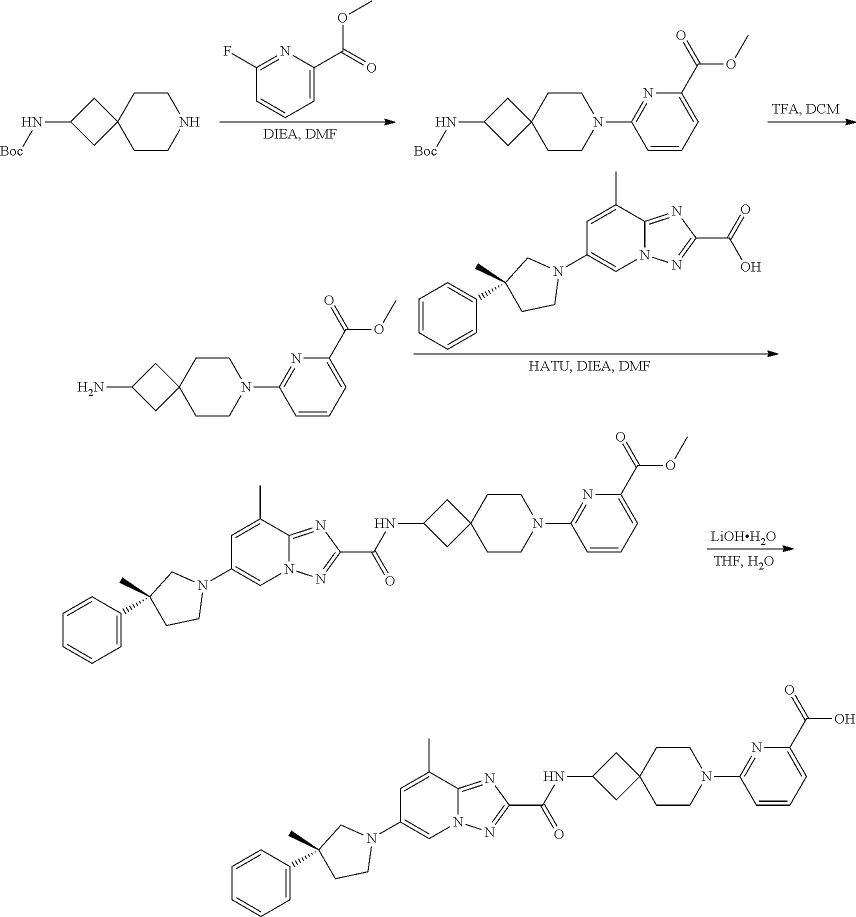

### Step 1: methyl 6-2-(tert-butoxycarbonylamino)-7-azaspiro [3.5]nonan-7-yl]pyridine-2-carboxylate

To a mixture of tert-butyl N-(7-azaspiro [3.5]nonan-2-yl)carbamate (500 mg, 2.08 mmol, 1 eq) and methyl 6-fluoropyridine-2-carboxylate (322.72 mg, 2.08 mmol, 1 eq) in DMF (2 mL) was added DIEA (806.62 mg, 6.24 mmol, 1.09 mL, 3 eq), and then the mixture was stirred at 100° C. for 16 h. LCMS showed starting material was consumed and one main peak with desired mass was detected. The mixture was filtered, and concentrated under pressure to give the product. The residue was purified by prep-HPLC purification (mobile phase: [water (FA)—ACN]; Br: 0%-70%, 6 min), the mixture was concentrated and lyophilized to give compound methyl 6-[2-(tert-butoxycarbonylamino)-7-azaspiro [3.5]nonan-7-yl]pyridine-2-carboxylate (413 mg, 1.09 mmol, 52.35 yield, 99 purity) as white solid.

LC-MS [ESI, M+1]: 376.1

### Step 2: methyl 6-(2-amino-7-azaspiro[3.5]nonan-7-yl)pyridine-2-carboxylate

To a mixture of methyl 6-[2-(tert-butoxycarbonylamino)-7-azaspiro[3.5]nonan-7-yl]pyridine-2-carboxylate (233 mg, 620.57 μmol, 1 eq) in DCM (2 mL) was added TFA (1.32 g, 11.62 mmol, 862.96 μL, 18.72 eq), and then the mixture was stirred at 25° C. for 0.5 h. LCMS showed starting material was consumed and one main peak with desired mass was detected. The reaction mixture was diluted with saturated sodium carbonate (10 mL) and extracted with ethyl acetate (30 mL). The combined organic layers was washed with saturated brine. It was dried over Na₂SO₄, filtered and concentrated under reduced pressure to give compound methyl 6-(2-amino-7-azaspiro [3.5]nonan-7-yl) pyri dine-2-carboxylate (140 mg, 508.45 μmol, 81.93% yield, 100% purity) as yellow liquid.

LC-MS [ESI, M+1]: 276.3

### Step 3: methyl 6-[2-[[8-methyl-6-1(3R)-3-methyl-3-phenyl-pyrrolidin-1-yl]-[1,2,4]triazolo[1,5-a]pyridine-2-carbonyl]amino]-7-azaspiro[3.5]nonan-7-yl]pyridine-2-carboxylate

To a mixture of 8-methyl-6-[(3R)-3-methyl-3-phenyl-pyrrolidin-1-yl]-[1,2,4]triazolo [1,5-a]pyridine-2-carboxylic acid (171.04 mg, 508.45 μmol, 1 eq) in DMF (1 mL) was added HATU (579.99 mg, 1.53 mmol, 3 eq), methyl 6-(2-amino-7-azaspiro[3.5]nonan-7-yl)pyridine-2-carboxylate (140 mg, 508.45 μmol, 1 eq) and DIEA (328.57 mg, 2.54 mmol, 442.82 μL, 5 eq), and then the mixture was stirred at 25° C. for 12 h. LCMS showed starting material was consumed and one main peak with desired mass was detected. The mixture was filtered, and concentrated under pressure to give the product. The residue was purified by prep-HPLC purification (mobile phase: [water (FA)—ACN]; B %: 30%-70%, 6 min), the mixture was concentrated and lyophilized to give compound methyl 6-[2-[[8-methyl-6-[(3R)-3-methyl-3-phenyl-pyrrolidin-1-yl]-[1,2,4]triazolo [1,5-a]pyridine-2-carbonyl]amino]-7-azaspiro[3.5]nonan-7-yl]pyridine-2-carboxylate (287 mg, 483.40 μmol, 95.07% yield) as light yellow gum.

LC-MS [ESI, M+1]: 594.2

### Step 4: 6-[2-[[8-methyl-6-[(3R)-3-methyl-3-phenyl-pyrrolidin-1-yl]-[1,2,4]triazolo[1,5-a]pyridine-2-carbonyl]amino]-7-azaspiro[3.5]nonan-7-yl]pyridine-2-carboxylic acid

To a mixture of methyl 6-[2-[[8-methyl-6-[(3R)-3-methyl-3-phenyl-pyrrolidin-1-yl]-[1,2,4]triazolo[1,5-a]pyridine-2-carbonyl]amino]-7-azaspiro[3.5]nonan-7-yl]pyridine-2-carboxylate (100 mg, 168.43 μmol, 1 eq) and LiOH·H₂O (28.27 mg, 673.72 μmol, 4 eq) in THF (1 mL) was added H₂O (0.1 mL), and then the mixture was stirred at 25° C. for 3 h. LCMS showed starting material was consumed and one main peak with desired mass was detected. The mixture was filtered, and concentrated under pressure to give the product. The residue was purified by prep-HPLC purification (column: Phenomenex luna C18 150*25 mm*10 um; mobile phase: [water(FA)—ACN]; gradient:5%-55% B over 10 min), the mixture was concentrated and lyophilized to give compound 6-[2-[[8-methyl-6-[(3R)-3-methyl-3-phenyl-pyrrolidin-1-yl]-[1,2,4]triazolo[1,5-a]pyridine-2-carbonyl]amino]-7-azaspiro[3.5]nonan-7-yl]pyridine-2-carboxylic acid (48.24 mg, 83.22 μmol, 49.41% yield, 100% purity) as white solid.

LC-MS [ESI, M+1]: 580.3

¹H NMR (400 MHz, CHLOROFORM-d) δ=7.72-7.62 (m, 2H), 7.56-7.48 (m, 2H), 7.42-7.36 (m, 2H), 7.36-7.32 (m, 2H), 7.28 (br d, J=1.2 Hz, 1H), 7.02 (s, 1H), 6.96 (d, J=8.8 Hz, 1H), 4.82-4.68 (m, 1H), 3.68-3.48 (m, 7H), 3.46-3.38 (m, 1H), 2.66 (s, 3H), 2.58-2.46 (m, 2H), 2.42 (td, J=8.4, 12.0 Hz, 1H), 2.34-2.24 (m, 1H), 1.98-1.90 (m, 2H), 1.84-1.80 (m, 2H), 1.74 (br d, J=5.6 Hz, 2H), 1.48 (s, 3H)

| Compound | | |
|---|---|---|
| Number | Synthesis | LC/MS |
| 643 | Similar to | m/z 566.2 |
| | Example 97 | [M + H]⁺ |
| 644 | Similar to | m/z 552.2 |
| | Example 97 | [M + H]⁺ |
| 645 | Similar to | m/z 552.3 |
| | Example 97 | [M + H]⁺ |
| 646 | Similar to | m/z 568.3 |
| | Example 97 | [M + H]⁺ |
| 647 | Similar to | m/z 554.4 |
| | Example 97 | [M + H]⁺ |
| 648 | Similar to | m/z 580.4 |
| | Example 97 | [M + H]⁺ |
| 649 | Similar to | m/z 568.3 |
| | Example 97 | [M + H]⁺ |
| 650 | Similar to | m/z 554.4 |
| | Example 97 | [M + H]⁺ |
| 651 | Similar to | m/z 552.2 |
| | Example 97 | [M + H]⁺ |
| 652 | Similar to | m/z 552.4 |
| | Example 97 | [M + H]⁺ |
| 653 | Similar to | m/z 538.3 |
| | Example 97 | [M + H]⁺ |
| 654 | Similar to | m/z 566.4 |
| | Example 97 | [M + H]⁺ |
| 655 | Similar to | m/z 540.2 |
| | Example 97 | [M + H]⁺ |
| 656 | Similar to | m/z 582.3 |
| | Example 97 | [M + H]⁺ |
| 657 | Similar to | m/z 552.2 |
| | Example 97 | [M + H]⁺ |
| 658 | Similar to | m/z 538.7 |
| | Example 97 | [M + H]⁺ |
| 659 | Similar to | m/z 568.3 |
| | Example 97 | [M + H]⁺ |
| 660 | Similar to | m/z 538.3 |
| | Example 97 | [M + H]⁺ |
| 661 | Similar to | m/z 565.68 |
| | Example 97 | [M + H]⁺ |
| 643 | Similar to | m/z 551.65 |
| | Example 97 | [M + H]⁺ |
| 644 | Similar to | m/z 553.67 |
| | Example 97 | [M + H]⁺ |
| 645 | Similar to | m/z 553.67 |
| | Example 97 | [M + H]⁺ |

### Example 98

### 6-((6-(8-methyl-6-((R)-3-methyl-3-phenylpyrrolidin-1-yl)-[1,2,4]triazolo[1,5-a]pyridine-2-carboxamido)spiro[3.3]heptan-1-yl)amino)picolinic acid (Compound 662)

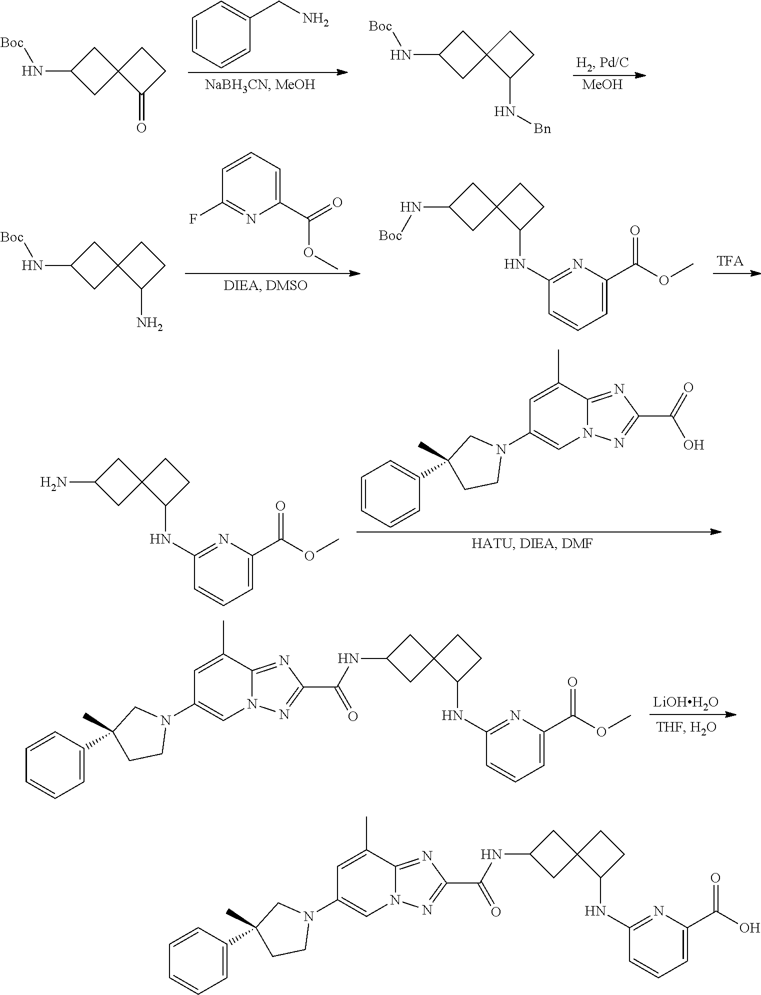

### Step 1: tert-butyl (5-(benzylamino)spiro[3.3]heptan-2-yl)carbamate

To a solution of tert-butyl N-(7-oxospiro[3.3]heptan-2-yl)carbamate (150 mg, 665.83 μmol, 1 eq) in MeOH (1 mL) was added NaBH₃CN (20.92 mg, 332.91 μmol, 0.5 eq) and phenylmethanamine (71.35 mg, 665.83 μmol, 72.58 μL, 1 eq). The mixture was stirred at 0° C. for 1 h. LCMS showed starting material was consumed and the desired mass was detected. The reaction mixture was concentrated under the reduced pressure to give the crude product. The residue was purified by prep-TLC (SiO₂, PE:EtOAc=0:1, Rf=0.70) to give compound tert-butyl N-[7-(benzylamino)spiro[3.3]heptan-2-yl]carbamate (200 mg, 632.04 μmol, 47.46% yield) as yellow oil.

LC-MS [ESI, M+1]: 317.2.

¹H NMR (400 MHz, CHLOROFORM-d) δ=7.42-7.27 (m, 4H), 7.25 (s, 1H), 4.86-4.44 (m, 1H), 4.15-3.98 (m, 1H), 3.81 (s, 1H), 3.09 (t, J=8.0 Hz, 1H), 2.90-2.77 (m, 1H), 2.49-2.32 (m, 1H), 2.20-1.99 (m, 2H), 1.88-1.73 (m, 2H), 1.69-1.60 (m, 4H), 1.45 (s, 9H).

### Step 2: tert-butyl (5-aminospiro[3.3]heptan-2-yl)carbamate

To a solution of tert-butyl N-[7-(benzylamino)spiro[3.3]heptan-2-yl]carbamate (200 mg, 632.04 μmol, 1 eq) in MeOH (5 mL) and THF (5 mL) was added Pd/C (250.00 mg, 234.92 μmol, 10% purity, 0.1 eq) under N₂ atmosphere. The suspension was degassed and purged with H₂ for 3 times. The mixture was stirred under H₂ (50 Psi) at 25° C. for 12 h. LCMS showed starting material was consumed and the desired mass was detected. The reaction mixture was filtered and the filtrate was concentrated under the reduced pressure to give the product. The residue was purified by prep-TLC (SiO₂, DCM:MeOH=10:1, Rf=0.50) to give compound tert-butyl N-(7-aminospiro[3.3]heptan-2-yl)carbamate (65 mg, 287.21 μmol, 45.44% yield) as yellow oil.

LC-MS [ESI, M+1]: 227.2.

¹H NMR (400 MHz, CHLOROFORM-d) δ=4.64 (br s, 1H), 3.99-3.80 (m, 1H), 2.77-2.66 (m, 1H), 2.39-2.28 (m, 1H), 2.17-2.07 (m, 1H), 1.84 (br dd, J=8.4, 11.6 Hz, 2H), 1.66-1.54 (m, 4H), 1.44 (s, 9H).

### Step 3: methyl 6-((6-((tert-butoxycarbonyl)amino)spiro[3.3]heptan-1-yl)amino)picolinate

To a solution of tert-butyl N-(7-aminospiro[3.3]heptan-2-yl)carbamate (55 mg, 243.02 μmol, 1 eq) in DMSO (1 mL) was added DIEA (94.23 mg, 729.07 μmol, 126.99 μL, 3 eq) and methyl 6-fluoropyridine-2-carboxylate (56.55 mg, 364.54 μmol, 1.5 eq). The mixture was stirred at 100° C. for 12 h. LCMS showed starting material was consumed and the desired mass was detected. The reaction mixture was extracted with ethyl acetate (15 mL×2). The combined organic phase was washed with brine (30 mL), dried with anhydrous Na₂S04, filtered and concentrated in vacuum. The residue was purified by prep-HPLC (FA condition column: Phenomenex luna C18 150*25 mm*10 um; mobile phase: [water(FA)—ACN]; gradient:28%-58% B over 10 min) to give compound methyl 6-[[2-(tert-butoxycarbonylamino)spiro[3.3]heptan-7-yl]amino]pyridine-2-carboxylate (20 mg, 55.34 μmol, 22.77% yield, 100% purity) as red solid.

LC-MS [ESI, M+1]: 362.2.

### Step 4: methyl 6-((6-aminospiro[3.3]heptan-1-yl)amino)picolinate

To a solution of methyl 6-[[2-(tert-butoxycarbonylamino)spiro[3.3]heptan-7-yl]amino]pyridine-2-carboxylate (20 mg, 55.34 μmol, 1 eq) in DCM (0.5 mL) was added TFA (383.75 mg, 3.37 mmol, 0.25 mL, 60.82 eq). The mixture was stirred at 25° C. for 0.5 h. LCMS showed starting material was consumed and the desired mass was detected. The reaction mixture was concentrated under the reduced pressure to give compound methyl 6-[(2-aminospiro[3.3]heptan-7-yl)amino]pyridine-2-carboxylate (35 mg, crude, TFA) as yellow oil.

LC-MS [ESI, M+1]: 262.0.

### Step 5: methyl 6-((6-(8-methyl-6-((R)-3-methyl-3-phenylpyrrolidin-1-yl)-[1,2,4]triazolo[1,5-a]pyridine-2-carboxamido)spiro[3.3]heptan-1-yl)amino)picolinate

To a solution of 8-methyl-6-[(3R)-3-methyl-3-phenyl-pyrrolidin-1-yl]-[1,2,4]triazolo[1,5-a]pyridine-2-carboxylic acid (15 mg, 44.59 μmol, 1 eq) in DMF (0.5 mL) was added methyl 6-[(2-aminospiro[3.3]heptan-7-yl)amino]pyridine-2-carboxylate (25.11 mg, 66.89 μmol, 1.5 eq, TFA), HATU (20.35 mg, 53.51 μmol, 1.2 eq) and DIEA (17.29 mg, 133.77 μmol, 23.30 μL, 3 eq). The mixture was stirred at 25° C. for 1 h. LCMS showed starting material was consumed and the desired mass was detected. The reaction mixture was concentrated under the reduced pressure to give the crude product. The residue was purified by prep-HPLC (FA condition column: Phenomenex luna C18 150*25 mm*10 um; mobile phase: [water(FA)—ACN]; gradient:48%-78% B over 15 min) to give compound methyl 6-[[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]amino]pyridine-2-carboxylate (15 mg, 25.88 μmol, 58.03% yield, 100% purity) as yellow solid.

LC-MS [ESI, M+1]: 580.3.

### Step 6: 6-((6-(8-methyl-6-((R)-3-methyl-3-phenylpyrrolidin-1-yl)-[1,2,4]triazolo[1,5-a]pyridine-2-carboxamido)spiro[3.3]heptan-1-yl)amino)picolinic acid

To a solution of methyl 6-[[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]amino]pyridine-2-carboxylate (15 mg, 25.88 μmol, 1 eq) in THF (1 mL) and H₂O (0.3 mL) was added LiOH·H₂O (3.26 mg, 77.63 μmol, 3 eq). The mixture was stirred at 25° C. for 1 h. LCMS showed starting material was consumed and the desired mass was detected. The reaction mixture was concentrated under the reduced pressure to give the crude product. The residue was purified by prep-HPLC (FA condition column: Phenomenex luna C18 150*25 mm*10 um; mobile phase: [water(FA)—ACN]; gradient:35%-65% B over 10 min) to give compound 6-[[2-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-7-yl]amino]pyridine-2-carboxylic acid (8.06 mg, 14.07 μmol, 54.38% yield, 98.746% purity) as white solid.

LC-MS [ESI, M+1]: 566.2.

¹H NMR (400 MHz, CHLOROFORM-d) δ=7.84 (br d, J=6.4 Hz, 1H), 7.63 (br s, 1H), 7.50 (br s, 2H), 7.41-7.34 (m, 2H), 7.33-7.29 (m, 2H), 7.26-7.21 (m, 1H), 6.98 (s, 1H), 6.88 (br d, J=8.4 Hz, 1H), 4.43-4.21 (m, 1H), 4.09 (br dd, J=2.4, 6.0 Hz, 1H), 3.59 (br d, J=8.8 Hz, 1H), 3.56-3.44 (m, 2H), 3.43-3.33 (m, 1H), 3.18 (br d, J=6.0 Hz, 1H), 2.74-2.65 (m, 1H), 2.62 (s, 3H), 2.37 (td, J=8.4, 11.6 Hz, 2H), 2.28-2.20 (m, 2H), 2.17-2.11 (m, 1H), 2.06-1.98 (m, 2H), 1.94-1.84 (m, 2H), 1.46 (s, 3H).

| Compound | | |
|---|---|---|
| Number | Synthesis | LC/MS |
| 663 | Similar to | m/z 566.3 |
| | Example 98 | [M + H]⁺ |
| 559 | Similar to | m/z 557.6 |
| | Example 98 | [M + H]⁺ |
| 600 | Similar to | m/z 529.6 |
| | Example 98 | [M + H]⁺ |

### Example 99

### 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylic acid (Compound 664)

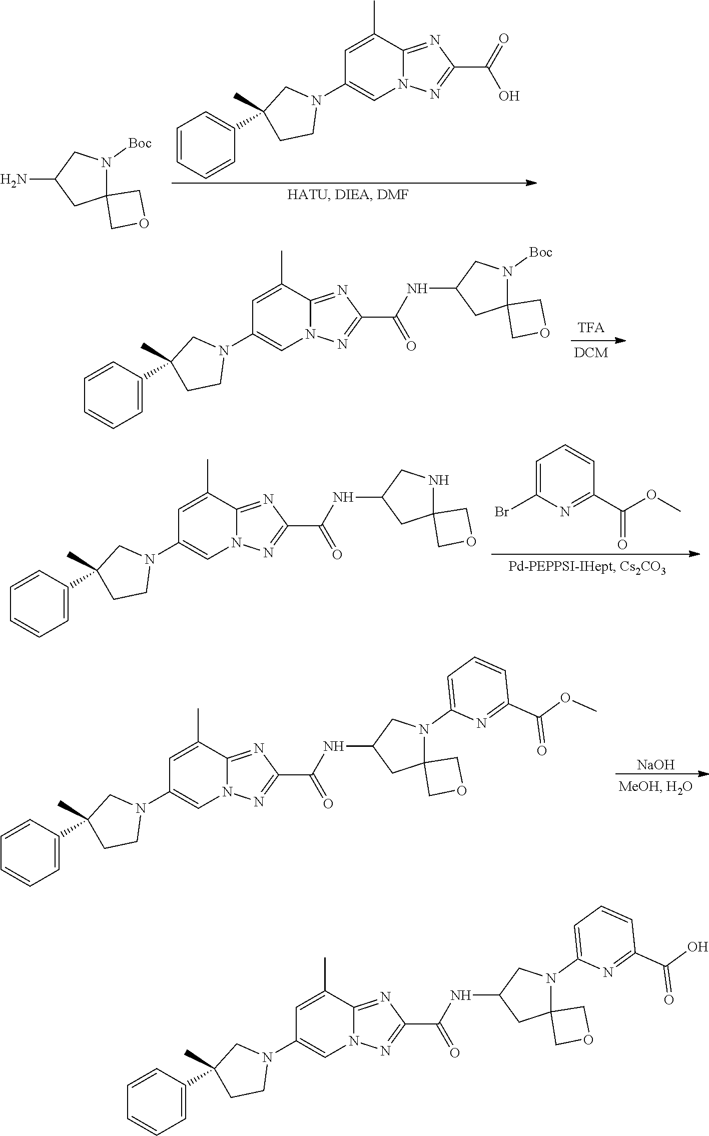

### Step 1: tert-butyl 7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octane-5-carboxylate

To a solution of 8-methyl-6-[(3R)-3-methyl-3-phenyl-pyrrolidin-1-yl]-[1,2,4]triazolo[1,5-a]pyridine-2-carboxylic acid (80 mg, 237.82 μmol, 1 eq) and tert-butyl 7-amino-2-oxa-5-azaspiro[3.4]octane-5-carboxylate (65.15 mg, 285.39 μmol, 1.2 eq) in DMF (1.5 mL) was added DIEA (92.21 mg, 713.46 μmol, 124.27 μL, 3 eq) and HATU (108.51 mg, 285.39 μmol, 1.2 eq). The mixture was stirred at 25° C. for 0.5 h. LCMS showed reactant was consumed completely and one main peak with desired mass was detected. The reaction was diluted with H₂O (3 mL) and extracted with EtOAc (6 mL×3). The combined organic layers were washed with brine (6 mL×2), dried over Na₂SO₄, filtered and the filtrate was concentrated under reduced pressure to give a residue. The residue was purified by reversed-phase HPLC (0.1% FA condition) to give tert-butyl 7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octane-5-carboxylate (115 mg, 210.37 u mol, 88.46% yield) as a white solid.

LC-MS [ESI, M+1]: 547.3

¹H NMR (400 MHz, CHLOROFORM-d) δ=7.65 (d, J=1.6 Hz, 1H), 7.39-7.27 (m, 5H), 7.01 (s, 1H), 5.63-5.39 (m, 1H), 5.30 (d, J=7.2 Hz, 1H), 4.68-4.41 (m, 3H), 3.86 (dd, J=6.4, 11.2 Hz, 1H), 3.68-3.51 (m, 2H), 3.50-3.38 (m, 2H), 2.74 (dd, J=5.6, 13.2 Hz, 1H), 2.63 (s, 3H), 2.39 (td, J=8.4, 12.4 Hz, 1H), 2.32-2.22 (m, 1H), 1.69-1.47 (m, 15H).

### Step 2: 8-methyl-6-[(3R)-3-methyl-3-phenyl-pyrrolidin-1-yl]-N-(2-oxa-5-azaspiro[3.4]octan-7-yl)-[1,2,4]triazolo[1,5-a]pyridine-2-carboxamide

To a solution of tert-butyl 7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octane-5-carboxylate (115 mg, 210.37 μmol, 1 eq) in DCM (1 mL) was added TFA (307.00 mg, 2.69 mmol, 0.2 mL, 12.80 eq). The mixture was stirred at 25° C. for 1 h. LCMS showed reactant was consumed completely and one main peak with desired mass was detected. The reaction was concentrated under reduced pressure to give a residue. The residue was purified by reversed-phase HPLC (neutral condition) to give 8-methyl-6-[(3R)-3-methyl-3-phenyl-pyrrolidin-1-yl]-N-(2-oxa-5-azaspiro[3.4]octan-7-yl)-[1,2,4]triazolo[1,5-a]pyridine-2-carboxamide (57 mg, 126.28 μmol, 60.03% yield, 98.927% purity) as a white solid.

LC-MS [ESI, M+1]: 447.3

### Step 3: methyl 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylate

A mixture of 8-methyl-6-[(3R)-3-methyl-3-phenyl-pyrrolidin-1-yl]-N-(2-oxa-5-azaspiro[3.4]octan-7-yl)-[1,2,4]triazolo[1,5-a]pyridine-2-carboxamide (32 mg, 71.66 μmol, 1 eq), methyl 6-bromopyridine-2-carboxylate (23.22 mg, 107.49 μmol, 1.5 eq), Cs₂CO₃ (70.05 mg, 214.98 μmol, 3 eq), [1,3-bis[2,6-bis(1-ethylpropyl)phenyl]-4,5-dichloro-imidazol-2-ylidene]-dichloro-(2-methylpyridin-1-ium-1-yl)palladium (6.02 mg, 7.17 μmol, 0.1 eq) in dioxane (1.5 mL) was degassed and purged with N₂ for 3 times, and then the mixture was stirred at 100° C. for 16 h under N₂ atmosphere. LCMS showed 13% of reactant 1 remained and 30% of desired mass was detected. The reaction was concentrated under reduced pressure to give a residue. The residue was purified by reversed-phase HPLC (0.1% FA condition) to give methyl 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylate (10 mg, 16.44 u mol, 22.95% yield, 95.647% purity) as a yellow solid.

LC-MS [ESI, M+1]: 582.3

### Step 4: 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylic acid

To a solution of methyl 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylate (10 mg, 17.19 μmol, 1 eq) in THF (0.4 mL) was added NaOH (2.06 mg, 51.58 μmol, 3 eq) in water (0.4 mL). The mixture was stirred at 25° C. for 0.5 h. LCMS showed reactant was consumed completely and one main peak with desired mass was detected. The reaction was concentrated under reduced pressure to give a residue. The residue was purified by prep-HPLC (column: CD24-WePure Biotech XPT C18 150×25×7 um; mobile phase: [H₂O (10 mM NH₄HCO₃)—ACN]; gradient:25%-55% B over 11.0 min) to give 6-[7-[[8-methyl-6-[(3R)-3-methyl-3-phenyl-pyrrolidin-1-yl]-[1,2,4]triazolo[1,5-a]pyridine-2-carbonyl]amino]-2-oxa-5-azaspiro[3.4]octan-5-yl]pyridine-2-carboxylic acid (5.16 mg, 9.06 μmol, 52.68% yield, 99.629% purity) as a white solid.

LC-MS [ESI, M+1]: 568.3

¹H NMR (400 MHz, METHANOL-d4) δ=8.20-7.99 (m, 0.38H), 7.88-7.72 (m, 1.52H), 7.60-7.45 (m, 1H), 7.43-7.31 (m, 3.65H), 7.31-7.15 (m, 2.50H), 7.01 (d, J=8.4 Hz, 0.63H), 5.57-5.37 (m, 1H), 5.26-5.11 (m, 0.7H), 5.04-4.98 (m, 0.3H), 4.84 (s, 1H), 4.66-4.58 (m, 1H), 4.27 (dd, J=8.0, 12.0 Hz, 0.24H), 4.04-3.84 (m, 1.3H), 3.80-3.71 (m, 0.5H), 3.70-3.50 (m, 4H), 3.41 (dt, J=4.0, 8.8 Hz, 1H), 2.81-2.64 (m, 1.30H), 2.63-2.54 (m, 3H), 2.53-2.48 (m, 0.25H), 2.43-2.17 (m, 0.26H), 1.52-1.36 (m, 3H).

| Compound | | |
|---|---|---|
| Number | Synthesis | LC/MS |
| 665 | Similar to | m/z 568.4 |
| | Example 99 | [M + H]⁺ |
| 666 | Similar to | m/z 556.4 |
| | Example 99 | [M + H]⁺ |
| 667 | Similar to | m/z 552.3 |
| | Example 99 | [M + H]⁺ |
| 668 | Similar to | m/z 552.5 |
| | Example 99 | [M + H]⁺ |

### Example 100

### 6-(8-(8-methyl-6-((R)-3-methyl-3-phenylpyrrolidin-1-yl)-[1,2,4]triazolo[1,5-a]pyridine-2-carboxamido)-6-oxa-2-azaspiro[3.5]nonan-2-yl)picolinic acid (Compound 669)

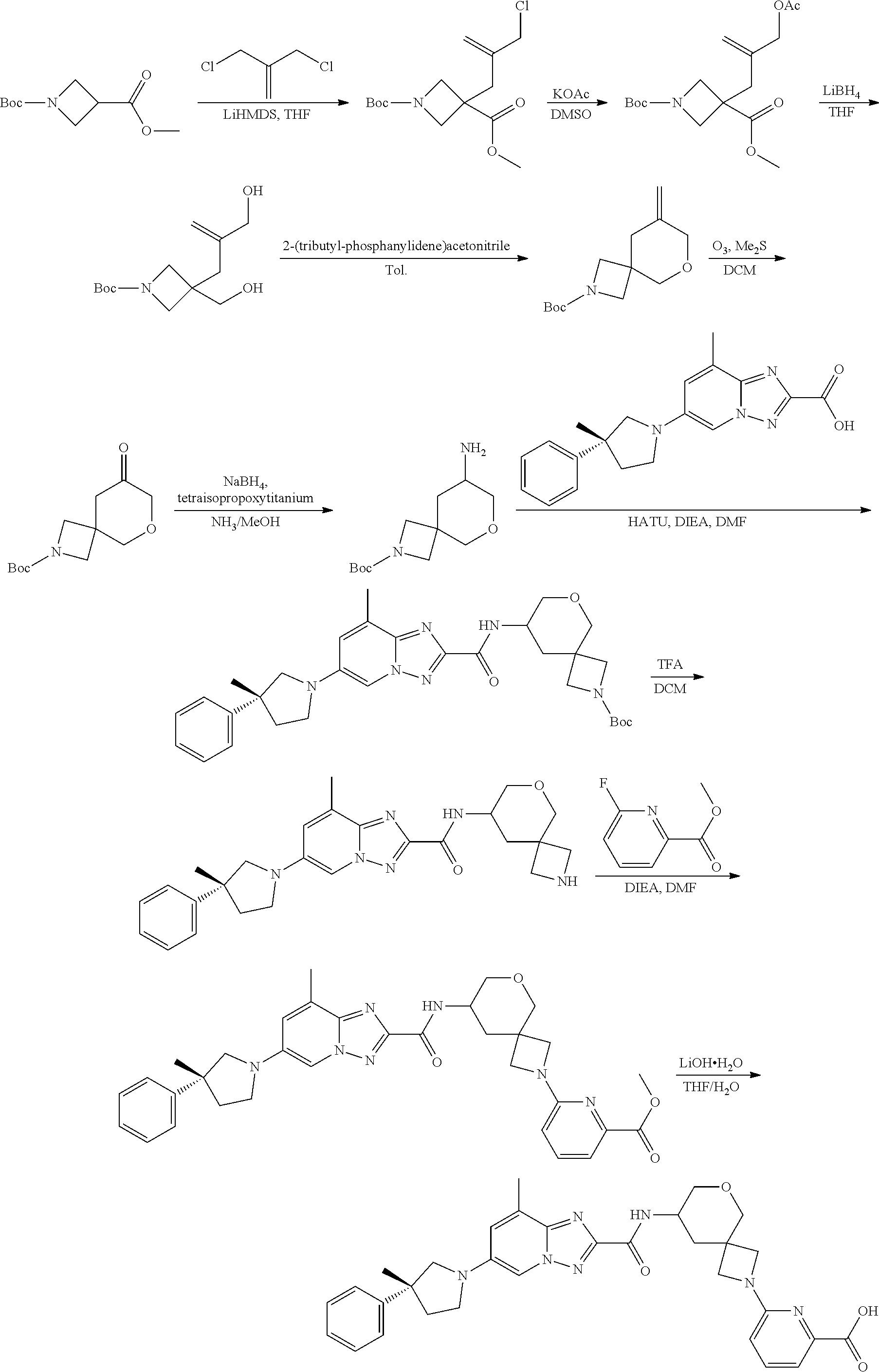

### Step 1: 1-(tert-butyl) 3-methyl 3-(2-(chloromethyl)allyl)azetidine-1,3-dicarboxylate

To a solution of O1-tert-butyl O3-methyl azetidine-1,3-dicarboxylate (5 g, 23.23 mmol, 1 eq) in THF (50 mL) was added LiHMDS (1 M, 23.23 mL, 1 eq) and 3-chloro-2-(chloromethyl)prop-1-ene (8.71 g, 69.69 mmol, 8.07 mL, 3 eq) at −78° C. The mixture was stirred at 25° C. for 1 h under N₂ atmosphere. TLC (SiO₂, PE:EtOAc=3:1, Rf=0.7) indicated reactant was consumed completely and one new spot was formed. The reaction was quenched by NH₄Cl (50 mL) slowly and then extracted with ethyl acetate (20 mL×3). The combined organic phase was washed with brine (10×3 mL), dried with anhydrous Na₂SO₄, filtered and concentrated in vacuum The residue was purified by column chromatography (SiO₂, PE:EtOAc=3:1, Rf=0.7) to give compound O1-tert-butyl O3-methyl 3-[2-(chloromethyl)allyl]azetidine-1,3-dicarboxylate (650 mg, 9% yield) as a yellow oil.

¹H NMR (400 MHz, CHLOROFORM-d) δ=5.43-5.27 (m, 1H), 5.19 (d, J=7.2 Hz, 1H), 4.89-4.75 (m, 1H), 4.27-4.20 (m, 2H), 4.01-3.96 (m, 1H), 3.83 (dd, J=3.6, 8.8 Hz, 2H), 3.72 (d, J=14.4 Hz, 3H), 2.88 (d, J=17.6 Hz, 2H), 1.44 (d, J=2.0 Hz, 9H).

### Step 2: 1-(tert-butyl) 3-methyl 3-(2-(acetoxymethyl)allyl)azetidine-1,3-dicarboxylate

To a solution of O1-tert-butyl O3-methyl 3-[2-(chloromethyl)allyl]azetidine-1,3-dicarboxylate (4.8 g, 15.80 mmol, 1 eq) in DMSO (50 mL) was added KOAc (3.10 g, 31.60 mmol, 2 eq). The mixture was stirred at 90° C. for 2 h. TLC (SiO₂, PE:EtOAc=3:1, Rf=0.3) indicated reactant was consumed and one new spot formed. The reaction was added water (10 mL) and then extracted with ethyl acetate (10 mL×3). The combined organic phase was washed with brine (5 mL×3), dried with anhydrous Na₂SO₄, filtered and concentrated in vacuum. The residue was purified by column chromatography (SiO₂, PE:EtOAc=3:1,Rf=0.3) to give compound O1-tert-butyl O3-methyl 3-[2-(acetoxymethyl)allyl]azetidine-1,3-dicarboxylate (950 mg, 18% yield) as a colourless oil.

¹H NMR (400 MHz, CHLOROFORM-d) δ=5.12 (s, 1H), 4.81 (s, 1H), 4.45 (s, 2H), 4.25 (br d, J=8.8 Hz, 2H), 3.78 (d, J=8.8 Hz, 2H), 3.74 (s, 3H), 2.74 (s, 2H), 2.09 (s, 3H), 1.44 (s, 9H).

### Step 3: tert-butyl 3-(hydroxymethyl)-3-(2-(hydroxymethyl)allyl)azetidine-1-carboxylate

To a solution of O1-tert-butyl O3-methyl 3-[2-(acetoxymethyl)allyl]azetidine-1,3-dicarboxylate (850 mg, 2.60 mmol, 1 eq) in THF (9 mL) was added dropwise LiBH₄ (2 M, 6.49 mL, 5 eq) at 0° C. The mixture was stirred at 25° C. for 1 h. TLC (SiO₂, PE:EtOAc=1:1, Rf=0.4) indicated reactant was consumed completely and some new spots formed. The reaction was quenched by NH₄Cl (20 mL) slowly and then extracted with ethyl acetate (10 mL×3). The combined organic phase was washed with brine (10 mL), dried with anhydrous Na₂SO₄, filtered and concentrated in vacuum. The residue was purified by column chromatography (SiO₂, PE:EtOAc=1:1,Rf=0.4) to give compound tert-butyl 3-(hydroxymethyl)-3-[2-(hydroxymethyl)allyl]azetidine-1-carboxylate (450 mg, 67% yield) as a colourless oil.

¹H NMR (400 MHz, CHLOROFORM-d) δ=5.12 (d, J=0.8 Hz, 1H), 4.89 (s, 1H), 4.06 (s, 2H), 3.69 (s, 6H), 2.49 (s, 2H), 1.43 (s, 9H).

### Step 4: tert-butyl 8-methylene-6-oxa-2-azaspiro[3.5]nonane-2-carboxylate

To a solution of tert-butyl 3-(hydroxymethyl)-3-[2-(hydroxymethyl)allyl]azetidine-1-carboxylate (400 mg, 1.55 mmol, 1 eq) in Tol. (4 mL) was added 2-(tributyl-phosphanylidene)acetonitrile (562.76 mg, 2.33 mmol, 1.5 eq). The mixture was stirred at 110° C. for 12 h. The reaction was monitored by TLC, TLC (PE:EtOAc=3:1) showed one new spot (Rf=0.6) was detected. The reaction was added water (10 mL) and then extracted with ethyl acetate (10 mL×3). The combined organic phase was washed with brine (10 mL), dried with anhydrous Na₂SO₄, filtered and concentrated in vacuum. The residue was purified by column chromatography (SiO₂, PE:EtOAc=3:1, Rf=0.6) to give compound tert-butyl 8-methylene-6-oxa-2-azaspiro[3.5]nonane-2-carboxylate (250 mg, 67% yield) as a yellow solid

¹H NMR (400 MHz, CHLOROFORM-d) δ=4.89 (d, J=8.4 Hz, 2H), 3.98 (s, 2H), 3.71 (s, 2H), 3.68-3.62 (m, 2H), 3.60-3.54 (m, 2H), 2.45 (s, 2H), 1.45 (s, 9H).

### Step 5: tert-butyl 8-oxo-6-oxa-2-azaspiro[3.5]nonane-2-carboxylate

A stream of OZONE (40.12 mg, 835.74 μmol, 1 eq) is passed through a cooled −78° C. solution of tert-butyl 8-methylene-6-oxa-2-azaspiro[3.5]nonane-2-carboxylate (200 mg, 835.74 μmol, 1 eq) in DCM (8 mL) until TLC analysis (PE:EtOAc=3:1) indicates that conversion of starting material (Rf=0.10) to a more polar product is complete. The reaction mixture is then quenched with Me₂S (207.70 mg, 3.34 mmol, 245.50 μL, 4 eq) stirred at −20° C. and stirred at 250 for 1 h. The reaction was monitored by TLC, TLC (PE:EtOAc=3:1) showed one new spot (Rf=0.10) was detected. The mixture was concentrated in vaco. compound tert-butyl 8-oxo-6-oxa-2-azaspiro[3.5]nonane-2-carboxylate (210 mg, crude) as a colourless oil.

### Step 6: tert-butyl 8-amino-6-oxa-2-azaspiro[3.5]nonane-2-carboxylate

To a solution of tert-butyl 8-oxo-6-oxa-2-azaspiro[3.5]nonane-2-carboxylate (210 mg, 870.35 μmol, 1 eq) in NH₃/MeOH (4 mL) was added tetraisopropoxytitanium (494.73 mg, 1.74 mmol, 513.74 μL, 2 eq). The mixture was stirred at 25° C. for 1 h, and then NaBH₄ (49.39 mg, 1.31 mmol, 1.5 eq) was added at 25° C. The mixture was stirred at 25° C. for 1 h. The reaction was monitored by TLC, TLC (PE:EtOAc=0:1) showed one new spot (Rf=0.10) was detected. The reaction was quenched by NH₄Cl (10 mL) slowly and then extracted with ethyl acetate (10 mL×3). The combined organic phase was washed with brine (5 mL), dried with anhydrous Na₂SO₄, filtered and concentrated in vacuum. The residue was purified by prep-TLC (SiO₂, PE:EtOAc=0:1, Rf=0.1) to give compound tert-butyl 8-amino-6-oxa-2-azaspiro[3.5]nonane-2-carboxylate (138 mg, 65% yield) as a yellow oil.

¹H NMR (400 MHz, CHLOROFORM-d) δ=4.22-3.49 (m, 6H), 3.35 (br d, J=11.6 Hz, 1H), 3.07-2.93 (m, 1H), 2.62 (s, 2H), 2.20 (br d, J=12.4 Hz, 1H), 1.44 (s, 9H).

### Step 7: tert-butyl 8-(8-methyl-6-((R)-3-methyl-3-phenylpyrrolidin-1-yl)-[1,2,4]triazolo[1,5-a]pyridine-2-carboxamido)-6-oxa-2-azaspiro[3.5]nonane-2-carboxylate

To a solution of tert-butyl 8-amino-6-oxa-2-azaspiro[3.5]nonane-2-carboxylate (126.78 mg, 523.21 μmol, 2.2 eq) and 8-methyl-6-[(3R)-3-methyl-3-phenyl-pyrrolidin-1-yl]-[1,2,4]triazolo[1,5-a]pyridine-2-carboxylic acid (80 mg, 237.82 μmol, 1 eq) in DMF (1.5 mL) was added HATU (180.85 mg, 475.64 μmol, 2 eq). The mixture was stirred at 25° C. for 0.5 h, and then DIEA (153.68 mg, 1.19 mmol, 207.12 μL, 5 eq) was added at 25° C. The mixture was stirred at 25° C. for 1 h. LCMS showed starting material was consumed and one main peak with desired mass was detected. The reaction was added water (5 mL) and then extracted with ethyl acetate (10 mL×3). The combined organic phase was washed with brine (5 mL×3), dried with anhydrous Na₂SO₄, filtered and concentrated in vacuum. The residue was purified by prep-TLC (SiO₂, PE:EtOAc=0:1,Rf=0.5) to give compound tert-butyl 8-[[8-methyl-6-[(3R)-3-methyl-3-phenyl-pyrrolidin-1-yl]-[1,2,4]triazolo[1,5-a]pyridine-2-carbonyl]amino]-6-oxa-2-azaspiro[3.5]nonane-2-carboxylate (125 mg, 93% yield) as a yellow gum.

LC-MS [ESI, M+1]: 561.4.

¹H NMR (400 MHz, CHLOROFORM-d) δ=7.66 (d, J=1.6 Hz, 1H), 7.42-7.30 (m, 5H), 7.27-7.22 (m, 1H), 7.01 (s, 1H), 4.37-4.20 (m, 1H), 3.97-3.87 (m, 1H), 3.80-3.69 (m, 6H), 3.61 (br d, J=8.8 Hz, 1H), 3.57-3.47 (m, 3H), 3.46-3.38 (m, 1H), 2.39 (td, J=8.4, 12.0 Hz, 1H), 2.32-2.23 (m, 1H), 2.22-2.07 (m, 2H), 2.03 (s, 3H), 1.47 (s, 3H), 1.45-1.40 (m, 9H).

### Step 8: 8-methyl-6-((R)-3-methyl-3-phenylpyrrolidin-1-yl)-N-(6-oxa-2-azaspiro[3.5]nonan-8-yl)-[1,2,4]triazolo[1,5-a]pyridine-2-carboxamide

To a solution of tert-butyl 8-[[8-methyl-6-[(3R)-3-methyl-3-phenyl-pyrrolidin-1-yl]-[1,2,4]triazolo[1,5-a]pyridine-2-carbonyl]amino]-6-oxa-2-azaspiro[3.5]nonane-2-carboxylate (125 mg, 222.94 μmol, 1 eq) in DCM (2 mL) was added TFA (1.54 g, 13.46 mmol, 1 mL, 60.38 eq). The mixture was stirred at 25° C. for 0.5 h. LCMS showed starting material was consumed and one main peak with desired mass was detected. The reaction mixture was concentrated under the reduced pressure to give compound 8-methyl-6-[(3R)-3-methyl-3-phenyl-pyrrolidin-1-yl]-N-(6-oxa-2-azaspiro[3.5]nonan-8-yl)-[1,2,4]triazolo[1,5-a]pyridine-2-carboxamide (55 mg, 43% yield, TFA) as a white solid.

LC-MS [ESI, M+1]: 461.2.

### Step 9: methyl 6-(8-(8-methyl-6-((R)-3-methyl-3-phenylpyrrolidin-1-yl)-[1,2,4]triazolo[1,5-a]pyridine-2-carboxamido)-6-oxa-2-azaspiro[3.5]nonan-2-yl)picolinate

To a solution of 8-methyl-6-[(3R)-3-methyl-3-phenyl-pyrrolidin-1-yl]-N-(6-oxa-2-azaspiro[3.5]nonan-8-yl)-[1,2,4]triazolo[1,5-a]pyridine-2-carboxamide (50 mg, 87.02 μmol, 1 eq, TFA) and methyl 6-fluoropyridine-2-carboxylate (27.00 mg, 174.04 μmol, 2 eq) in DMSO (1 mL) was added K₂CO₃ (36.08 mg, 261.05 μmol, 3 eq). The mixture was stirred at 100° C. for 1 h. LCMS showed starting material was consumed and one main peak with desired mass was detected. The reaction was added water (5 mL) and then extracted with ethyl acetate (10 mL×3). The combined organic phase was washed with brine (5 mL×3), dried with anhydrous Na₂SO₄, filtered and concentrated in vacuum. The residue was purified by column chromatography (SiO₂, PE:EtOAc=0:1, Rf=0.4) to give compound methyl 6-[8-[[8-methyl-6-[(3R)-3-methyl-3-phenyl-pyrrolidin-1-yl]-[1,2,4]triazolo[1,5-a]pyridine-2-carbonyl]amino]-6-oxa-2-azaspiro[3.5]nonan-2-yl]pyridine-2-carboxylate (12 mg, 23% yield) as a white solid.

LC-MS [ESI, M+1]: 596.2.

### Step 10: 6-(8-(8-methyl-6-((R)-3-methyl-3-phenylpyrrolidin-1-yl)-[1,2,4]triazolo[1,5-a]pyridine-2-carboxamido)-6-oxa-2-azaspiro[3.5]nonan-2-yl)picolinic acid

To a solution of methyl 6-[8-[[8-methyl-6-[(3R)-3-methyl-3-phenyl-pyrrolidin-1-yl]-[1,2,4]triazolo[1,5-a]pyridine-2-carbonyl]amino]-6-oxa-2-azaspiro[3.5]nonan-2-yl]pyridine-2-carboxylate (12 mg, 20.14 μmol, 1 eq) in THF (1 mL) was added LiOH (1.93 mg, 80.58 μmol, 4 eq) in H₂O (0.3 mL) was added at 25° C. The mixture was stirred at 25° C. for 0.5 h. LCMS showed starting material was consumed and one main peak with desired mass was detected. The reaction mixture was poured into 2 mL water and acidified with 2M HCl to pH around 4 and then the reaction mixture was concentrated under the reduced pressure to give the product. The residue was purified by prep-HPLC (NH₄HCO₃ condition column: CD02-Waters Xbidge BEH C18 150*25*10 um; mobile phase: [water(NH₄HCO₃)—ACN]; gradient:25%-55% B over 11 min) to give compound 6-[8-[[8-methyl-6-[(3R)-3-methyl-3-phenyl-pyrrolidin-1-yl]-[1,2,4]triazolo[1,5-a]pyridine-2-carbonyl]amino]-6-oxa-2-azaspiro[3.5]nonan-2-yl]pyridine-2-carboxylic acid (4.15 mg, 7.13 μmol, 35.42% yield, 100% purity) as a white solid.

LC-MS [ESI, M+1]: 582.2.

¹H NMR (400 MHz, CHLOROFORM-d) δ=7.74-7.55 (m, 2H), 7.50 (br d, J=6.4 Hz, 1H), 7.45-7.27 (m, 6H), 7.02 (br s, 1H), 6.50 (br d, J=7.2 Hz, 1H), 4.34 (br s, 1H), 3.98 (br d, J=8.4 Hz, 2H), 3.92-3.81 (m, 4H), 3.72-3.32 (m, 6H), 2.64 (br s, 3H), 2.43-2.35 (m, 1H), 2.31-2.10 (m, 3H), 1.47 (s, 3H)

### Example 101

### 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid (Compound 670)

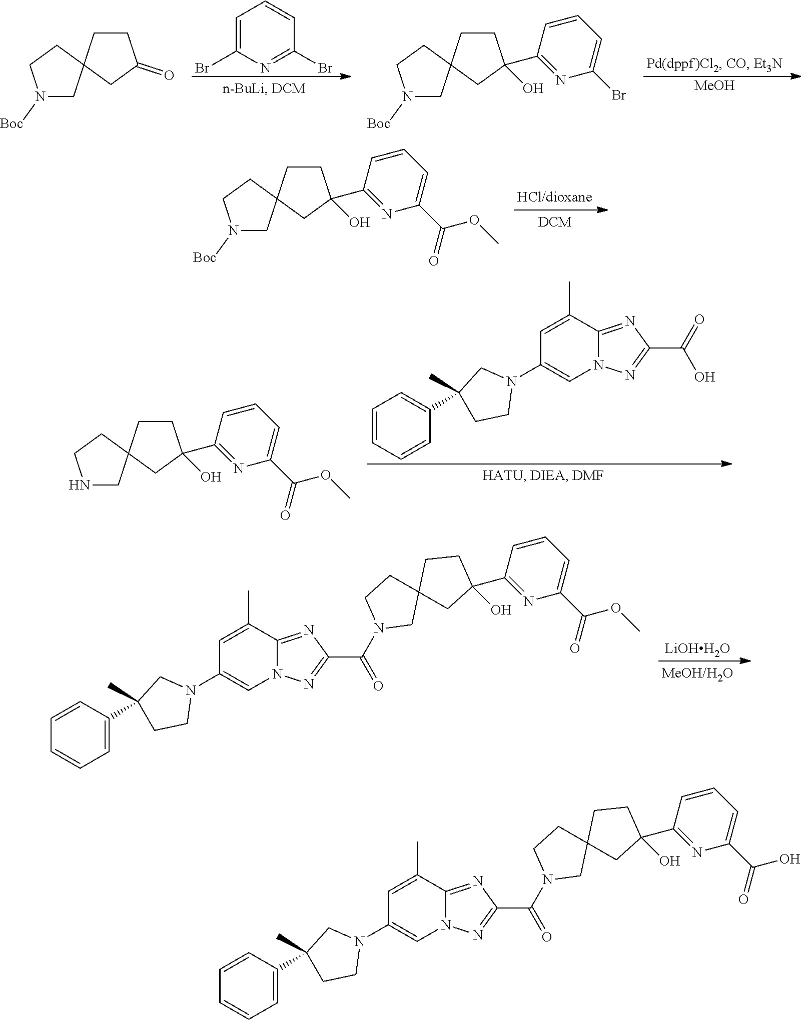

### Step 1: tert-butyl 8-(6-bromo-2-pyridyl)-8-hydroxy-2-azaspiro[4.4]nonane-2-carboxylate

To a solution of 2,6-dibromopyridine (449.95 mg, 1.90 mmol, 1 eq) in DCM (5 mL) was added dropwise n-BuLi (2.5 M, 1.14 mL, 1.5 eq) at −78° C. under N₂ atmosphere. After addition, the mixture was stirred at this temperature for 1 h, and then tert-butyl 8-oxo-2-azaspiro[4.4]nonane-2-carboxylate (500 mg, 2.09 mmol, 1.1 eq) was added at −78° C. and stirred for 0.5 h. Then the resulting mixture was warmed to 25° C. and stirred at 25° C. for 0.5 h. LCMS showed starting material was consumed. One main peak was shown on LCMS and 69% of desired compound was detected. The reaction mixture was quenched with NH₄Cl (5 mL) and then diluted with H₂O (10 mL) and extracted with DCM (15 mL×3). The combined organic layers were washed with brine 15 mL (5 mL×3), dried over Na₂SO₄, filtered and concentrated under reduced pressure to give a residue. The residue was purified by flash silica gel chromatography (SiO₂, PE:EtOAc=1:1, Rf=0.4) to give compound tert-butyl 8-(6-bromo-2-pyridyl)-8-hydroxy-2-azaspiro[4.4]nonane-2-carboxylate (138 mg, 18.29% yield) as yellow oil.

LC-MS [ESI, M-55]: 341.9.

¹H NMR (400 MHz, CHLOROFORM-d) δ=7.64-7.51 (m, 1H), 7.48-7.31 (m, 2H), 3.51-3.21 (m, 4H), 2.28-2.06 (m, 3H), 2.05-1.82 (m, 5H), 1.48 (s, 9H)

### Step 2: tert-butyl 8-hydroxy-8-(6-methoxycarbonyl-2-pyridyl)-2-azaspiro[4.4]nonane-2-carboxylate

To a solution of tert-butyl 8-(6-bromo-2-pyridyl)-8-hydroxy-2-azaspiro[4.4]nonane-2-carboxylate (138 mg, 347.34 μmol, 1 eq) in MeOH (15 mL) was added cyclopentyl(diphenyl)phosphane;dichloromethane;dichloropalladium;iron (28.37 mg, 34.73 μmol, 0.1 eq) and Et₃N (105.44 mg, 1.04 mmol, 145.04 μL, 3 eq) under N₂ atmosphere. The suspension was degassed and purged with CO for 3 times. The mixture was stirred under CO (50 Psi) at 80° C. for 12 h. LCMS showed starting material was consumed. Several new peaks were shown on LCMS and 48% of desired compound was detected. The reaction mixture was filtered and concentrated under reduced pressure to give a residue. The residue was purified by flash silica gel chromatography (SiO₂, PE:EtOAc=1:1,Rf=0.35) to give compound tert-butyl 8-hydroxy-8-(6-methoxycarbonyl-2-pyridyl)-2-azaspiro[4.4]nonane-2-carboxylate (112 mg, 81.63% yield) as yellow oil.

LC-MS [ESI, M-55]: 321.2.

¹H NMR (400 MHz, CHLOROFORM-d) δ=8.02 (dd, J=0.8, 7.6 Hz, 1H), 7.87 (t, J=7.8 Hz, 1H), 7.58 (dd, J=8.0, 12.8 Hz, 1H), 5.15 (s, 1H), 4.83 (s, 1H), 3.99 (s, 3H), 3.47-3.22 (m, 4H), 2.24-2.04 (m, 7H), 1.95-1.84 (m, 1H), 1.48 (s, 9H)

### Step 3: methyl 6-(8-hydroxy-2-azaspiro[4.4]nonan-8-yl)pyridine-2-carboxylate

To a solution of tert-butyl 8-hydroxy-8-(6-methoxycarbonyl-2-pyridyl)-2-azaspiro[4.4]nonane-2-carboxylate (40 mg, 106.26 μmol, 1 eq) in DCM (0.5 mL) was added HCl/dioxane (1.5 mL). The mixture was stirred at 25° C. for 0.5 h. LCMS showed starting material was consumed. One main peak was shown on LCMS and 93% of desired compound was detected. The reaction mixture was concentrated under reduced pressure to give a crude methyl 6-(8-hydroxy-2-azaspiro[4.4]nonan-8-yl)pyridine-2-carboxylate (30 mg, 67.26% yield, TFA) as yellow oil.

LC-MS [ESI, M+1]: 277.2.

### Step 4: methyl 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (25 mg, 74.32 μmol, 1 eq) in DMF (0.5 mL) was added methyl 6-(8-hydroxy-2-azaspiro[4.4]nonan-8-yl)pyridine-2-carboxylate (29.01 mg, 74.32 μmol, 1 eq, TFA) and DIEA (38.42 mg, 297.28 μmol, 51.78 μL, 4 eq). After addition, the mixture was stirred at this temperature for 5 min, and then HATU (42.39 mg, 111.48 μmol, 1.5 eq) was added. The resulting mixture was stirred at 25° C. for 25 min. LCMS showed starting material consumed. Several new peaks were shown on LCMS and 81% of desired compound was detected. The reaction mixture was diluted with H₂O (5 mL) and extracted with DCM (10 mL×3). The combined organic layers were washed with brine (5 mL×3), dried over Na₂SO₄, filtered and concentrated under reduced pressure to give a residue. The residue was purified by prep-TLC (SiO₂, PE:EtOAc=2:1, Rf=0.5) and eluted with EtOAc (50 mL) to give the product methyl 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate (18 mg, 40.32% yield) as white oil.

LC-MS [ESI, M+1]: 595.3.

### Step 5: 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid

To a solution of methyl 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylate (18 mg, 30.27 μmol, 1 eq) in MeOH (1 mL) and H₂O (0.3 mL) was added LiOH·H₂O (5.08 mg, 121.07 μmol, 4 eq). The mixture was stirred at 25° C. for 0.5 h. LCMS showed starting material consumed. One main peak was shown on LCMS and 96% of desired compound was detected. The mixture was adjusted pH to 5 with FA and concentrated under reduced pressure to give a residue. The residue was purified by prep-HPLC (FA condition; column: CD01-Phenomenex luna C18 150*25*10 um; mobile phase: [water(FA)—ACN]; gradient:355%-65% B over 10 min) to give compound 6-[8-hydroxy-2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]pyridine-2-carboxylic acid (11.72 mg, 65.35% yield) as white solid.

LC-MS [ESI, M+1]: 581.2.

¹H NMR (400 MHz, CHLOROFORM-d) δ=8.26-8.07 (m, 1H), 8.06-7.90 (m, 1H), 7.84-7.63 (m, 1H), 7.47-7.27 (m, 5H), 7.25 (br d, J=2.0 Hz, 1H), 7.01 (br s, 1H), 4.24-3.31 (m, 8H), 2.65 (br s, 3H), 2.47-1.90 (m, 10H), 1.48 (s, 3H).

| Compound | | |
|---|---|---|
| Number | Synthesis | LC/MS |
| 670 | Example | m/z 580.69 |
| | 101 | [M + H]⁺ |

### Example 102

### 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylic acid (Compound 671)

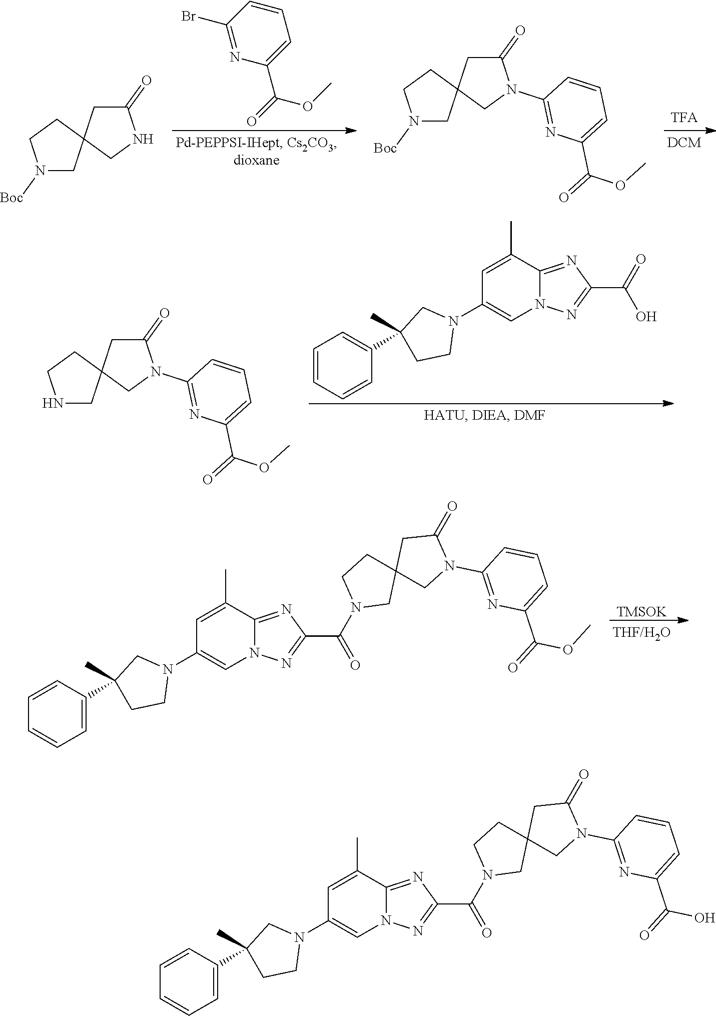

### Step 1: tert-butyl 2-(6-methoxycarbonyl-2-pyridyl)-3-oxo-2,7 diazaspiro[4.4]nonane-7-carboxylate

A mixture of tert-butyl 8-oxo-2,7-diazaspiro[4.4]nonane-2-carboxylate (250 mg, 1.04 mmol, 1.1 eq),methyl 6-bromopyridine-2-carboxylate (204.32 mg, 945.80 μmol, 1 eq), Cs₂CO₃ (924.48 mg, 2.84 mmol, 3 eq), 1,3-bis[2,6-bis(1-propylbutyl)phenyl]-4,5-dichloro-2H-imidazol-1-ium-2-ide;3-chloropyridine;dichloropalladium (46.00 mg, 47.29 μmol, 0.05 eq) in dioxane (5 mL) was degassed and purged with N₂ for 3 times, and then the mixture was stirred at 110° C. for 12 h under N₂ atmosphere. LCMS showed starting material was consumed and the desired mass was detected. The reaction solution was concentrated under the reduced pressure to give the residue. The residue was purified by lash silica gel chromatography (SiO₂, EtOAc:PE=10:1, Rf=0.5) to give tert-butyl 2-(6-methoxycarbonyl-2-pyridyl)-3-oxo-2,7-diazaspiro[4.4]nonane-7-carboxylate (250 mg, 70.41% yield) as a yellow solid.

LC-MS [ESI, M+1]: 376.1

¹H NMR (400 MHz, CHLOROFORM-d) δ=8.59 (t, J=7.6 Hz, 1H), 7.90-7.81 (m, 2H), 4.19-4.08 (m, 2H), 3.98 (s, 3H), 3.59-3.45 (m, 3H), 3.40 (d, J=14.0 Hz, 1H), 2.78-2.62 (m, 2H), 2.08-1.92 (m, 2H), 1.47 (d, J=4.4 Hz, 9H)

### Step 2: methyl 6-(3-oxo-2,7-diazaspiro[4.4]nonan-2-yl)pyridine-2-carboxylate

To a solution of tert-butyl 2-(6-methoxycarbonyl-2-pyridyl)-3-oxo-2,7-diazaspiro[4.4]nonane-7-carboxylate (100 mg, 266.37 μmol, 1 eq) in DCM (1.5 mL) was added TFA (1.54 g, 13.46 mmol, 1 mL, 50.54 eq). The mixture was stirred at 25° C. for 1 h. LCMS showed starting material was consumed and the desired mass was detected. The reaction solution was concentrated under the reduced pressure to give compound methyl 6-(3-oxo-2,7-diazaspiro[4.4]nonan-2-yl)pyridine-2-carboxylate (103 mg, crude, TFA) as a yellow solid.

LC-MS [ESI, M+1]: 276.2.

### Step 3: methyl 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (45 mg, 133.77 μmol, 1 eq) in DMF (1 mL) was added HATU (101.73 mg, 267.55 μmol, 2 eq) at 0° C. After addition, the mixture was stirred at this temperature for 0.5 h. and then methyl 6-(3-oxo-2,7-diazaspiro[4.4]nonan-2-yl)pyridine-2-carboxylate (62.50 mg, 160.53 μmol, 1.2 eq, TFA) and DIEA (86.45 mg, 668.87 μmol, 116.50 μL, 5 eq) was added dropwise at 0° C. The resulting mixture was stirred at 25° C. for 1 h. LCMS showed starting material was consumed and the desired mass was detected. The mixture was quenched with H₂O (2 mL) and extracted with EtOAc (2 mL×3), the organic phase was dried, filtered, and concentrated under pressure to give the product. The residue was purified by prep-TLC (SiO₂, EtOAc:NH₃·MeOH (7 M)=10:1, Rf=0.5) to give compound methyl 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylate (60 mg, 75.55% yield) as a yellow gum.

LC-MS [ESI, M+1]: 594.3.

### Step 4: 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylic acid

To a solution of methyl 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylate (50 mg, 84.22 μmol, 1 eq) in THF (1 mL) was added TMSOK (32.41 mg, 252.66 μmol, 3 eq). The mixture was stirred at 0° C. for 0.3 h. LCMS showed starting material was consumed and the desired mass was detected. Acidify the clear solution with FA (0.1 ml).The reaction solution was concentrated under the reduced pressure to give the residue. The residue was purified by prep-HPLC (column: CD04-Welch Utimate C18 150*25*7 um; mobile phase: [H₂O(FA)—ACN]; gradient:33%-63% B over 10 min) and lyophilized to give compound 6-[7-[8-methyl-6-[(3R)-3-methyl-3-phenyl-pyrrolidin-1-yl]-[1,2,4]triazolo[1,5-a]pyridine-2-carbonyl]-3-oxo-2,7-diazaspiro[4.4]nonan-2-yl]pyridine-2-carboxylic acid (19.33 mg, 38.56% yield) as a white solid.

LC-MS [ESI, M+1]: 580.3.

¹H NMR (400 MHz, DMSO-d₆) δ=8.43 (dd, J=7.6, 17.2 Hz, 1H), 8.04-7.85 (m, 2H), 7.77 (t, J=8.0 Hz, 1H), 7.43-7.30 (m, 4H), 7.28 (d, J=12.0 Hz, 1H), 7.25-7.17 (m, 1H), 4.10-4.01 (m, 2H), 3.95 (d, J=11.6 Hz, 1H), 3.86 (d, J=11.2 Hz, 1H), 3.69 (d, J=6.0 Hz, 1H), 3.59 (d, J=9.2 Hz, 1H), 3.49 (d, J=8.8 Hz, 2H), 3.39-3.31 (m, 2H), 2.85-2.67 (m, 2H), 2.54 (s, 3H), 2.24 (q, J=6.8 Hz, 2H), 2.16-1.96 (m, 2H), 1.33 (d, J=9.2 Hz, 3H).

### Example 103

### 2-hydroxy-2-methyl-4-(2-(8-methyl-6-((R)-3-methyl-3-phenylpyrrolidin-1-yl)-[1,2,4]triazolo[1,5-a]pyridine-2-carboxamido)-6-azaspiro[3.4]octan-6-yl)-4-oxobutanoic acid (Compound 673)

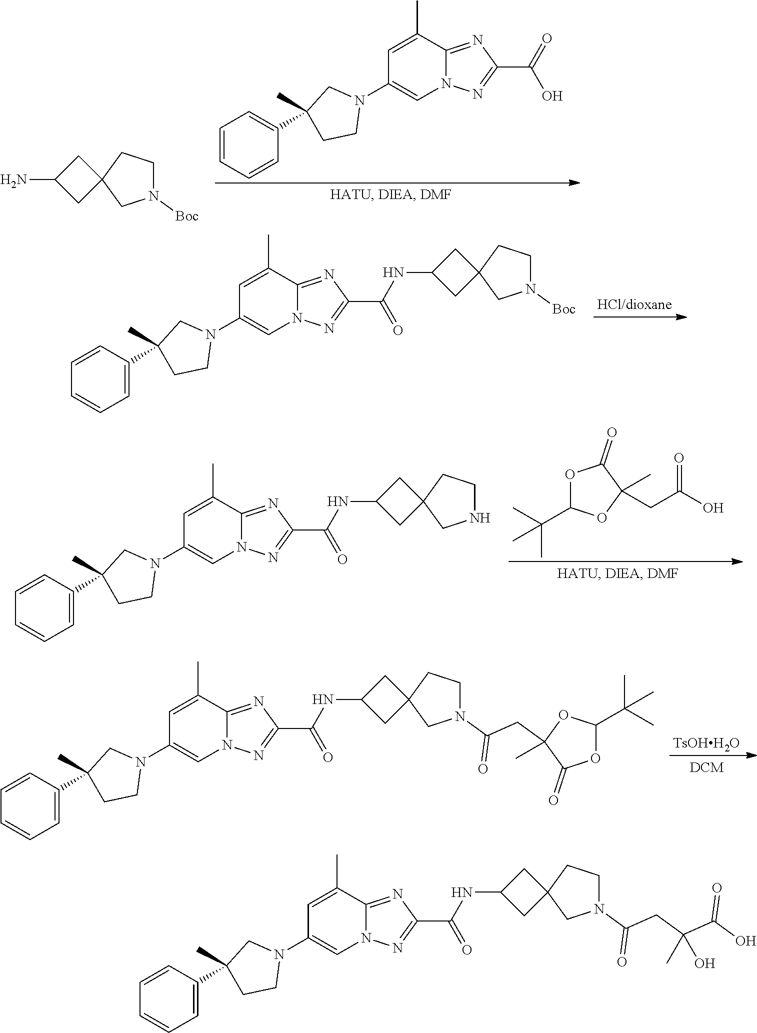

### Step 1: tert-butyl (R)-2-(8-methyl-6-(3-methyl-3-phenylpyrrolidin-1-yl)-[1,2,4]triazolo[1,5-a]pyridine-2-carboxamido)-6-azaspiro[3.4]octane-6-carboxylate

To a solution of tert-butyl 2-amino-6-azaspiro[3.4]octane-6-carboxylate (67.28 mg, 297.28 μmol, 1 eq) and 8-methyl6-[(3R)-3-methyl-3-phenyl-pyrrolidin-1-yl]-[1,2,4]triazolo[1,5-a]pyridine-2-carboxylic acid (100 mg, 297.28 μmol, 1 eq) in DMF (3 mL) was added HATU (226.07 mg, 594.55 μmol, 2 eq) and DIEA (115.26 mg, 891.83 μmol, 155.34 μL, 3 eq) The mixture was stirred at 25° C. for 1 h. LCMS showed starting material was consumed and one main peak with desired mass was detected. The reaction mixture was added H₂O (10 ml) and then was extracted with EtOAc (10 mL×3), The combined organic layers were washed with brine (30 mL×3), dried over anhydrous Na₂SO₄, filtered and dried to give a residue. The residue was purified by flash silica gel chromatography (PE:EtOAc=0 1 Rf=0.5) to give compound tert-butyl (R)-2-(8-methyl-6-(3-methyl-3-phenylpyrrolidin-1-yl)-[1,2,4]triazolo[1,5-a]pyridine-2-carboxamido)-6-azaspiro[3.4]octane-6-carboxylate (140 mg, 257.03 μmol, 86.46% yield) as a white gum.

LC-MS [ESI, M+1]: 545.5

¹H NMR (CHLOROFORM-d, 400 MHz): δ=7.43-7.75 (m, 2H), 7.28-7.39 (m, 3H), 7.22 (d, J=7.2 Hz, 1H), 6.98 (s, 1H), 4.45-4.75 (m, 1H), 3.41-3.76 (m, 3H), 3.20-3.40 (m, 5H), 2.53-2.62 (m, 3H), 2.35-2.41 (m, 1H), 2.30 (s, 1H), 2.16-2.25 (m, 1H), 1.98-2.06 (m, 3H), 1.80-1.93 (m, 2H), 1.38-1.49 (m, 12H)

### Step 2: tert-butyl (R)-2-(8-methyl-6-(3-methyl-3-phenylpyrrolidin-1-yl)-[1,2,4]triazolo[1,5-a]pyridine-2-carboxamido)-6-azaspiro[3.4]octane-6-carboxylate

To a solution of tert-butyl 2-[[8-methyl-6-[(3R)-3-methyl-3-phenyl-pyrrolidin-1-yl]-[1,2,4]triazolo[1,5-a]pyridine-2-carbonyl]amino]-6-azaspiro[3.4]octane-6-carboxylate (140 mg, 257.03 μmol, 1 eq) in DCM (2 mL) was added HCl/dioxane (2 M, 128.51 μL, 1 eq). The mixture was stirred at 25° C. for 1 h. LCMS showed starting material was consumed and one main peak with desired mass was detected. The reaction mixture was concentrated under the reduced pressure to give compound tert-butyl (R)-2-(8-methyl-6-(3-methyl-3-phenylpyrrolidin-1-yl)-[1,2,4]triazolo[1,5-a]pyridine-2-carboxamido)-6-azaspiro[3.4]octane-6-carboxylate (110 mg, crude, HCl) as a white gum.

LC-MS [ESI, M+1]: 445.2

### Step 3: N-(6-(2-(2-(tert-butyl)-4-methyl-5-oxo-1,3-dioxolan-4-yl)acetyl)-6-azaspiro[3.4]octan-2-yl)-8-methyl-6-((R)-3-methyl-3-phenylpyrrolidin-1-yl)-[1,2,4]triazolo[1,5-a]pyridine-2-carboxamide

To a solution of (R)-8-methyl-6-(3-methyl-3-phenylpyrrolidin-1-yl)-N-(6-azaspiro[3.4]octan-2-yl)-[1,2,4]triazolo[1,5-a]pyridine-2-carboxamide (45 mg, 101.22 μmol, 1 eq) and 2-(2-(tert-butyl)-4-methyl-5-oxo-1,3-dioxolan-4-yl)acetic acid (43.77 mg, 202.44 μmol, 2 eq) in DMF (3 mL) was added HATU (76.97 mg, 202.44 μmol, 2 eq) and DIEA (39.25 mg, 303.66 μmol, 52.89 μL, 3 eq). The mixture was stirred at 25° C. for 1 h. LCMS showed starting material was consumed and one main peak with desired mass was detected. The reaction mixture was added H₂O (10 ml) and then was extracted with EtOAc (10 mL×3), the combined organic layers were washed with brine (30 mL×3), dried over anhydrous Na₂SO₄, filtered and dried to give a residue. The residue was purified by prep-TLC (SiO₂, PE:EtOAc=0:1, Rf=0.3) and eluted with 60 mL (10% methanol in DCM) to give compound N-(6-(2-(2-(tert-butyl)-4-methyl-5-oxo-1,3-dioxolan-4-yl)acetyl)-6-azaspiro[3.4]octan-2-yl)-8-methyl-6-((R)-3-methyl-3-phenylpyrrolidin-1-yl)-[1,2,4]triazolo[1,5-a]pyridine-2-carboxamide (60 mg, 93.34 μmol, 92.22% yield) as a yellow solid.

LC-MS [ESI, M+1]: 643.4

### Step 4: 2-hydroxy-2-methyl-4-(2-(8-methyl-6-((R)-3-methyl-3-phenylpyrrolidin-1-yl)-[1,2,4]triazolo[1,5-a]pyridine-2-carboxamido)-6-azaspiro[3.4]octan-6-yl)-4-oxobutanoic acid

To a solution of N-[6-[2-(2-tert-butyl-4-methyl-5-oxo-1,3-dioxolan-4-yl)acetyl]-6-azaspiro[3.4]octan-2-yl]-8-methyl6-[(3R)-3-methyl-3-phenyl-pyrrolidin-1-yl]-[1,2,4]triazolo[1,5-a]pyridine-2-carboxamide (45 mg, 70.01 μmol, 1 eq) in DCM (3 mL) was added TsOH·H₂O (39.95 mg, 210.02 μmol, 3 eq). The mixture was stirred at 50° C. for 1 h. LCMS showed starting material was consumed and one main peak with desired mass was detected. The residue was purified by prep-HPLC (column: CD01-Phenomenex luna C18 150*25*10 um; mobile phase: [water(FA)—ACN]; gradient:36%-66% B over 10 min) and (column: CD18-Welch Utimate C18 150*40*7 um; mobile phase: [water(FA)—ACN]; gradient:33%-63% B over 20 min) and lyophilized to give compound 2-hydroxy-2-methyl-4-(2-(8-methyl-6-((R)-3-methyl-3-phenylpyrrolidin-1-yl)-[1,2,4]triazolo[1,5-a]pyridine-2-carboxamido)-6-azaspiro[3.4]octan-6-yl)-4-oxobutanoic acid (1.58 mg, 2.61 μmol, 3.73% yield, 95.068% purity) as a white solid.

LC-MS [ESI, M+1]: 575.4

¹H NMR (CHLOROFORM-d, 400 MHz): δ=7.66 (s, 1H), 7.45-7.56 (m, 1H), 7.37 (d, J=7.2 Hz, 2H), 7.30-7.34 (m, 2H), 7.25 (br s, 1H), 7.02 (s, 1H), 4.62-4.84 (m, 1H), 3.51-3.65 (m, 4H), 3.38-3.51 (m, 4H), 3.00 (t, J=17.2 Hz, 1H), 2.64 (s, 3H), 2.46-2.62 (m, 3H), 2.35-2.44 (m, 1H), 2.08-2.32 (m, 5H), 2.03 (br t, J=6.8 Hz, 1H), 1.48 (d, J=8.0 Hz, 6H)

| Compound | | |
|---|---|---|
| Number | Synthesis | LC/MS |
| 672 | Similar to | m/z 587.4 |
| | Example 103 | [M + H]⁺ |

### Example 104

### 4-(2-(8-methyl-6-((R)-3-methyl-3-phenylpyrrolidin-1-yl)-[1,2,4]triazolo[1,5-a]pyridine-2-carbonyl)-2-azaspiro[4.4]nonan-7-yl)cyclohexane-1-carboxylic acid (Compound 674)

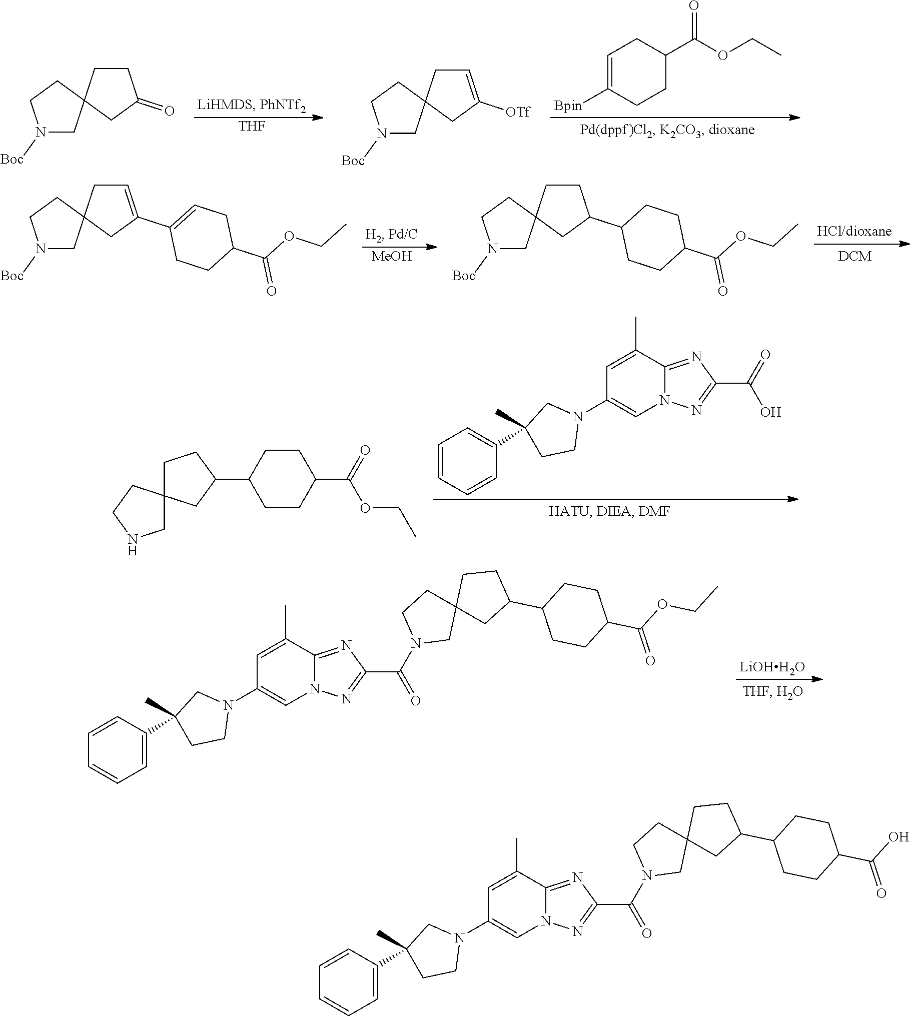

### Step 1: tert-butyl 8-(trifluoromethylsulfonyloxy)-2-azaspiro[4.4]non-7-ene-2-carboxylate

To a solution of LiHMDS (1 M, 2.51 mL, 2 eq) in THF (6 mL) was added tert-butyl 8-oxo-2-azaspiro[4.4]nonane-2-carboxylate (300 mg, 1.25 mmol, 1 eq) in THF (2.4 mL) dropwise at −78° C. and the mixture was stirred at −78° C. for 1 h under N₂. Then 1,1,1-trifluoro-N-phenyl-N-(trifluoromethylsulfonyl)methanesulfonamide (671.78 mg, 1.88 mmol, 1.5 eq) in THF (3.6 mL) was added and the reaction was warmed to 25° C. and stirred for 1 h under N₂. TLC indicated no starting material was remained, and one major new spot with lower polarity was detected. The mixture was quenched with saturated solution of NH₄Cl (5 mL) and extracted with ethyl acetate (3 mL×3), the combined organic phase was dried over anhydrous sodium sulfate, filtered and concentrated to give a residue. The residue was purified by flash silica gel chromatography (SiO₂, PE:EtOAc=10:1, Rf=0.30) to give tert-butyl 8-(trifluoromethylsulfonyloxy)-2-azaspiro[4.4]non-7-ene-2-carboxylate (370 mg, 996.31 μmol, 79.5% yield) as yellow oil

¹H NMR (400 MHz, METHANOL-d4) δ=5.69 (s, 1H), 3.52-3.34 (m, 2H), 3.29-3.21 (m, 2H), 2.76-2.65 (m, 2H), 2.03 (t, J=7.2 Hz, 3H), 1.85 (td, J=3.6, 7.2 Hz, 1H), 1.46 (s, 9H).

### Step 2: tert-butyl 8-(4-ethoxycarbonylcyclohexen-1-yl)-2-azaspiro[4.4]non-7-ene-2-carboxylate

To a flask equipped with a magnetic stir bar was added tert-butyl 8-(trifluoromethylsulfonyloxy)-2-azaspiro[4.4]non-7-ene-2-carboxylate (300 mg, 807.82 μmol, 1 eq), ethyl 4-(4,4,5,5-tetramethyl-1,3,2-dioxaborolan-2-yl)cyclohex-3-ene-1-carboxylate (271.59 mg, 969.38 μmol, 1.2 eq), Pd(dppf)C12 (29.55 mg, 40.39 μmol, 0.05 eq), K₂CO₃ (279.11 mg, 2.02 mmol, 2.5 eq). The flask was evacuated and backfilled with nitrogen three times. Dioxane (3 mL) were added and reaction was stirred at 80° C. for 3 h under N₂ atmosphere. LCMS showed starting material was consumed and one peak with desired mass was detected. The mixture was quenched with water (10 mL) and extracted with ethyl acetate (6 mL×3), the combined organic phase was dried over anhydrous sodium sulfate, filtered and concentrated to give a residue. The residue was purified by flash silica gel chromatography (SiO₂, PE:EtOAc=5:1, Rf=0.40) to give tert-butyl 8-(4-ethoxycarbonylcyclohexen-1-yl)-2-azaspiro[4.4]non-7-ene-2-carboxylate (120 mg, 319.57 μmol, 39.56% yield) as yellow oil.

LC-MS [ESI, M-55]: 320.1

¹H NMR (400 MHz, CHLOROFORM-d) δ=5.70 (br s, 1H), 5.45 (s, 1H), 4.13 (q, J=7.2 Hz, 2H), 3.55-3.05 (m, 4H), 2.57-2.45 (m, 3H), 2.42-2.29 (m, 3H), 2.27-2.01 (m, 2H), 1.92-1.62 (m, 5H), 1.44 (br d, J=5.2 Hz, 9H), 1.24 (t, J=7.2 Hz, 3H).

### Step 3: tert-butyl 8-(4-ethoxycarbonylcyclohexyl)-2-azaspiro[4.4]nonane-2-carboxylate

To a solution of tert-butyl 8-(4-ethoxycarbonylcyclohexen-1-yl)-2-azaspiro[4.4]non-7-ene-2-carboxylate (130 mg, 346.20 μmol, 1 eq) in EtOAc (5 mL) was added Pd/C (36.84 mg, 34.62 μmol, 10% purity, 0.1 eq), Pd(OH)₂ (48.62 mg, 34.62 μmol, 10% purity, 0.1 eq). The mixture was stirred under H₂ atmosphere (50 Psi) at 25° C. for 12 h. LCMS showed starting material was consumed and one peak with desired mass was detected. The mixture was filtered and purified by prep-TLC (SiO₂, PE:EtOAc=5:1, Rf=0.47) to give tert-butyl 8-(4-ethoxycarbonylcyclohexyl)-2-azaspiro[4.4]nonane-2-carboxylate (93 mg, 245.04 μmol, 70.8% yield) as colorless oil.

LC-MS [ESI, M-55]: 324.2.

¹H NMR (400 MHz, CHLOROFORM-d) δ=4.21-4.05 (m, 2H), 3.45-3.27 (m, 2H), 3.26-3.05 (m, 2H), 2.58-2.11 (m, 1H), 1.97 (br d, J=12.4 Hz, 2H), 1.91-1.63 (m, 7H), 1.59-1.50 (m, 2H), 1.46 (s, 9H), 1.45-1.32 (m, 2H), 1.31-1.20 (m, 5H), 1.20-0.88 (m, 3H).

### Step 4: ethyl 4-(2-azaspiro[4.4]nonan-8-yl)cyclohexanecarboxylate

To a solution of tert-butyl 8-(4-ethoxycarbonylcyclohexyl)-2-azaspiro[4.4]nonane-2-carboxylate (45 mg, 118.57 μmol, 1 eq) in DCM (0.5 mL) was added HCl/dioxane (4M, 592.84 μL, 20 eq). The mixture was stirred at 25° C. for 1 h. LCMS showed starting material was consumed and the desired mass was detected. The mixture was concentrated to give ethyl 4-(2-azaspiro[4.4]nonan-8-yl)cyclohexanecarboxylate (30 mg, 94.97 μmol, 80.1% yield, HCl) as white solid.

LC-MS [ESI, M+1]: 280.2.

### Step 5: ethyl 4-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]cyclohexanecarboxylate

To a solution of ethyl 4-(2-azaspiro[4.4]nonan-8-yl)cyclohexanecarboxylate (30 mg, 94.97 μmol, 1 eq, HCl), 8-methyl-6-[(3R)-3-methyl-3-phenyl-pyrrolidin-1-yl]-[1,2,4]triazolo[1,5-a]pyridine-2-carboxylic acid (31.95 mg, 94.97 μmol, 1 eq) in DMF (0.5 mL) was added HATU (54.17 mg, 142.46 μmol, 1.5 eq), DIEA (61.37 mg, 474.87 μmol, 82.71 μL, 5 eq). The mixture was stirred at 25° C. for 1 h. LCMS showed starting material was consumed and the desired mass was detected. The crude product was purified by reversed-phase HPLC (neutral) to give ethyl 4-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]cyclohexanecarboxylate (55 mg, 92.01 μmol, 96.9% yield) as yellow oil.

LC-MS [ESI, M+1]: 598.3.

¹H NMR (400 MHz, CHLOROFORM-d) δ=7.66 (s, 1H), 7.41-7.28 (m, 3H), 7.27 (s, 2H), 6.99 (s, 1H), 4.17-3.96 (m, 2H), 3.83-3.69 (m, 1H), 3.64-3.39 (m, 3H), 2.68-2.62 (m, 2H), 2.54-2.14 (m, 2H), 2.02-1.66 (m, 7H), 1.65-1.45 (m, 8H), 1.45-1.22 (m, 13H), 1.02-0.83 (m, 2H).

### Step 6: 4-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]cyclohexanecarboxylic acid

To a solution of ethyl 4-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]cyclohexanecarboxylate (35 mg, 58.55 μmol, 1 eq) in THF (0.4 mL), H₂O (0.2 mL) was added LiOH·H₂O (7.37 mg, 175.65 μmol, 3 eq). The mixture was stirred at 25° C. for 1 h. LCMS showed starting material was consumed and the desired mass was detected. The mixture was added FA to pH=3, then filtered. The residue was purified by prep-HPLC (column: CD01-Phenomenex luna C18 150*25*10 um; mobile phase: [water(FA)—ACN]; gradient:59%-89% B over 10 min) to give 4-[2-[8-methyl-6-[(3R)-3-methyl-3-phenyl-pyrrolidin-1-yl]-[1,2,4]triazolo[1,5-a]pyridine-2-carbonyl]-2-azaspiro[4.4]nonan-8-yl]cyclohexanecarboxylic acid (12.88 mg, 22.38 μmol, 38.2% yield, 99% purity) as white solid.

LC-MS [ESI, M+1]: 570.3.

¹H NMR (400 MHz, METHANOL-d4) δ=7.77 (br d, J=5.6 Hz, 1H), 7.44-7.28 (m, 4H), 7.22 (br s, 2H), 3.99 (br s, 1H), 3.87-3.64 (m, 2H), 3.55 (br s, 5H), 2.58 (s, 3H), 2.53-2.10 (m, 3H), 2.04-1.74 (m, 8H), 1.74-1.46 (m, 4H), 1.42 (s, 3H), 1.38-0.86 (m, 6H).

### Example 105

### 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinic acid (Compound 675)

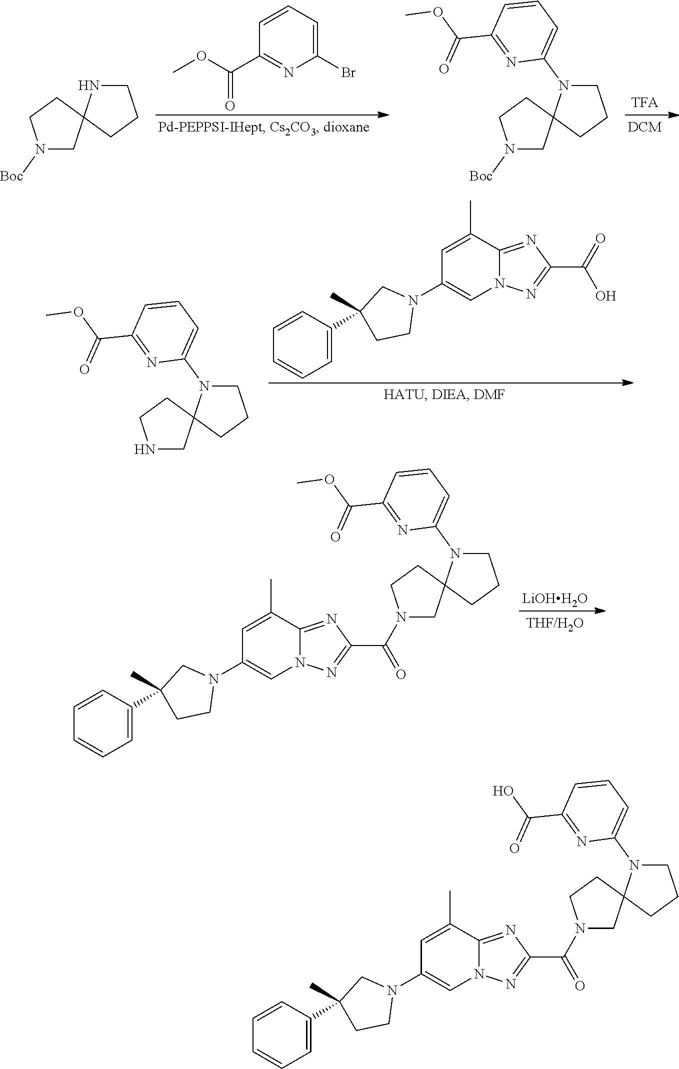

### Step 1: tert-butyl 1-(6-(methoxycarbonyl)pyridin-2-yl)-1,7-diazaspiro[4.4]nonane-7-carboxylate

To a solution of tert-butyl 1,7-diazaspiro[4.4]nonane-7-carboxylate (300 mg, 1.33 mmol, 1 eq) and methyl 6-bromopyridine-2-carboxylate (572.74 mg, 2.65 mmol, 2 eq) in dioxane (3 mL) was added 1,3-bis[2,6-bis(1-propylbutyl)phenyl]-4,5-dichloro-2H-imidazol-1-ium-2-ide;3-chloropyridine;dichloropalladium (12.89 mg, 13.26 μmol, 0.01 eq) and Cs₂CO₃ (1.30 g, 3.98 mmol, 3 eq). The mixture was stirred at 100° C. for 12 h. LCMS showed starting material was consumed and the desired mass was detected. The reaction mixture was concentrated under the reduced pressure to give the product. The residue was purified by prep-TLC (SiO₂, PE:EtOAc=0:1, Rf=0.3) and eluted with 60 mL (10% methanol in DCM) to give compound tert-butyl 1-(6-(methoxycarbonyl)pyridin-2-yl)-1,7-diazaspiro[4.4]nonane-7-carboxylate (150 mg, 415.01 μmol, 31.31% yield) as a white gum.

LC-MS [ESI, M+1]: 362.2

¹H NMR (CHLOROFORM-d, 400 MHz): δ=7.39-7.49 (m, 1H), 7.28 (d, J=6.0 Hz, 1H), 6.45 (d, J=8.4 Hz, 1H), 4.00-4.16 (m, 1H), 3.82 (s, 3H), 2.96-3.45 (m, 5H), 1.62-2.13 (m, 5H), 1.46 (s, 1H), 1.40 (d, J=14.4 Hz, 9H)

### Step 2: methyl 6-(1,7-diazaspiro[4.4]nonan-1-yl)picolinate

To a solution of tert-butyl 1-(6-methoxycarbonyl-2-pyridyl)-1,7-diazaspiro[4.4]nonane-7-carboxylate (100 mg, 276.68 μmol, 1 eq) in DCM (3 mL) was added TFA (31.55 mg, 276.68 μmol, 20.55 μL, 1 eq). The mixture was stirred at 25° C. for 1 h. LCMS showed starting material was consumed and the desired mass was detected. The reaction mixture was concentrated under the reduced pressure to give compound methyl 6-(1,7-diazaspiro[4.4]nonan-1-yl)picolinate (102 mg, 271.75 μmol, 98.22% yield, TFA) as a white gum.

LC-MS [ESI, M+1]: 262.1

### Step 3: methyl 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinate

To a solution of methyl 6-(1,7-diazaspiro[4.4]nonan-1-yl)picolinate (66.95 mg, 178.37 μmol, 2 eq, TFA) and 8-methyl-6-[(3R)-3-methyl-3-phenyl-pyrrolidin-1-yl]-[1,2,4]triazolo[1,5-a]pyridine-2-carboxylic acid (30 mg, 89.18 μmol, 1 eq) in DMF (3 mL) was added HATU (67.82 mg, 178.37 μmol, 2 eq) and DIEA (34.58 mg, 267.55 μmol, 46.60 μL, 3 eq). The mixture was stirred at 25° C. for 1 h. LCMS showed starting material was consumed and the desired mass was detected. The reaction mixture was added H₂O (10 ml) and then was extracted with EtOAc (10 mL×3), The combined organic layers were washed with brine (30 mL×3), dried over anhydrous Na₂SO₄, filtered and dried to give a residue. The residue was purified by prep-TLC (SiO₂, PE:EtOAc=0:1, Rf=0.4) and eluted with 60 mL (10% methanol in DCM) to give compound methyl 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinate (50 mg, 86.25 μmol, 96.71% yield) as a white gum.

LC-MS [ESI, M+1]: 580.2

### Step 4: 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinic acid

To a solution of methyl 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinate (35 mg, 60.38 μmol, 1 eq) in THF (3 mL) and MeOH (0.5 mL) and H₂O (0.5 mL) was added LiOH·H₂O (7.60 mg, 181.13 μmol, 3 eq). The mixture was stirred at 25° C. for 1 h. LCMS showed starting material was consumed and the desired mass was detected. The reaction mixture was concentrated under the reduced pressure to give the product. The residue was purified by prep-HPLC (column: CD25-XPT PHS C18 150*25*10 um; mobile phase: [water(FA)—ACN]; gradient:49%-79% B over 10 min) and lyophilized to give compound 6-(7-(8-methyl-6-((R)-3-methyl-3-phenylpyrrolidin-1-yl)-[1,2,4]triazolo[1,5-a]pyridine-2-carbonyl)-1,7-diazaspiro[4.4]nonan-1-yl)picolinic acid (11.55 mg, 20.42 μmol, 33.82% yield, 100% purity) as a white solid.

LC-MS [ESI, M+1]: 566.2

¹H NMR (CHLOROFORM-d, 400 MHz): δ=7.57-7.76 (m, 2H), 7.51 (d, J=7.2 Hz, 1H), 7.29-7.43 (m, 4H), 7.26 (s, 1H), 6.98-7.07 (m, 1H), 6.73 (dd, J=14.4, 8.8 Hz, 1H), 4.89-5.01 (m, 0.51H), 4.74 (t, J=10.0 Hz, 0.44H), 4.57 (d, J=13.2 Hz, 0.45H), 4.27 (t, J=11.2 Hz, 0.51H), 4.03 (td, J=11.6, 7.6 Hz, 0.50H), 3.88 (d, J=12.4 Hz, 0.55H), 3.64-3.79 (m, 1H), 3.38-3.63 (m, 5H), 3.02-3.25 (m, 1H), 2.57-2.70 (m, 3H), 2.34-2.41 (m, 1H), 2.19-2.32 (m, 3H), 2.04-2.11 (m, 2H), 1.71-1.81 (m, 2H), 1.47 (d, J=13.6 Hz, 3H)

### Example 107

### 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetic acid

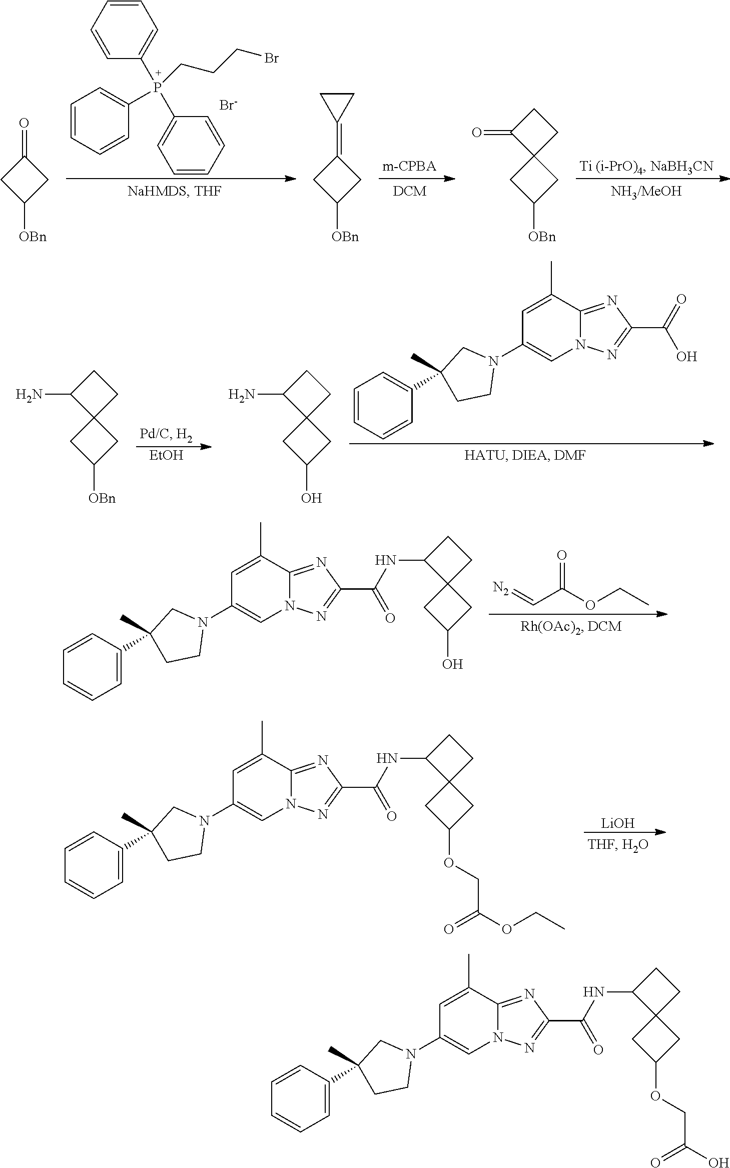

### Step 1: (3-cyclopropylidenecyclobutoxy)methylbenzene

To a solution of NaHMDS (1 M, 283.75 mL, 2.5 eq) in THF (250 mL) was added dropwise 3-bromopropyl(triphenyl)phosphonium;bromide (79.03 g, 170.25 mmol, 1.5 eq) at 25° C. under N₂ atmosphere. After addition, the mixture was stirred at 25° C. for 0.5 h, and then 3-benzyloxycyclobutanone (20 g, 113.50 mmol, 1 eq) was added dropwise at 80° C. The resulting mixture was stirred at 70° C. for 2 h. TLC indicated reactant was consumed completely and one new spot was formed. The reaction mixture was quenched by H₂O (250 mL), then extracted with ethyl acetate (100 mL×3), the combined organic layers dried over anhydrous sodium sulfate, filtered and the filtrate was concentrated under reduced pressure to give a residue. The residue was purified by column chromatography (SiO₂, hexanes:EtOAc=3:1, Rf=0.39) to give compound (3-cyclopropylidenecyclobutoxy)methylbenzene (4.5 g, 20% yield) as yellow oil.

¹H NMR (400 MHz, CHLOROFORM-d) δ=7.15-7.13 (m, 5H), 4.31 (s, 2H), 4.03 (t, J=6.4 Hz, 1H), 2.90-2.76 (m, 2H), 2.73-2.57 (m, 2H), 0.89-0.80 (m, 4H).

### Step 2: 2-benzyloxyspiro[3.3]heptan-7-one

To a solution of (3-cyclopropylidenecyclobutoxy)methylbenzene (1.3 g, 6.49 mmol, 1 eq) in DCM (20 mL) was added m-CPBA (1.98 g, 9.74 mmol, 85% purity, 1.5 eq) at 0° C. The mixture was stirred at 25° C. for 12 h. TLC indicated reactant was consumed completely and one new spot was formed. The reaction mixture was quenched by Saturated sodium sulfite solution (50 mL), then extracted with DCM (25 mL×3), the combined organic layers dried over anhydrous sodium sulfate, filtered and the filtrate was concentrated under reduced pressure to give a residue. The residue was purified by column chromatography (SiO₂, hexanes:EtOAc=3:1, Rf=0.30) to give compound 2-benzyloxyspiro[3.3]heptan-7-one (410 mg, 29% yield) as yellow oil.

¹H NMR (400 MHz, CHLOROFORM-d) δ=7.31-7.16 (m, 5H), 4.30 (s, 2H), 4.00 (t, J=7.2 Hz, 1H), 2.94-2.77 (m, 2H), 2.56-2.48 (m, 1H), 2.35-2.19 (m, 1H), 2.07-1.85 (m, 4H).

### Step 3: 2-benzyloxyspiro[3.3]heptan-7-amine

To a solution of 2-benzyloxyspiro[3.3]heptan-7-one (400 mg, 1.85 mmol, 1 eq) was added dropwise NH₃/MeOH (5 mL) and Ti(i-PrO)₄ (1.05 g, 3.70 mmol, 1.09 mL, 2 eq). After addition, the mixture was stirred at 25° C. for 10 h, and then NaBH₄ (104.96 mg, 2.77 mmol, 1.5 eq) was added dropwise at 0° C. The resulting mixture was stirred at 25° C. for 1 h. LCMS showed starting material was consumed and the desired mass was detected. The reaction mixture was quenched by H₂O (5 mL), then extracted with DCM (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.33) to give compound 2-benzyloxyspiro[3.3]heptan-7-amine (410 mg, 29% yield) as yellow gum.

LC-MS [ESI, M+1]: 218.3.

¹H NMR (400 MHz, CHLOROFORM-d) δ=7.36-7.27 (m, 5H), 4.44-4.32 (m, 2H), 4.11-4.00 (m, 1H), 3.46-3.37 (m, 1H), 2.43-2.34 (m, 1H), 2.31-2.12 (m, 2H), 2.12-1.75 (m, 5H).

### Step 4: 7-aminospiro[3.3]heptan-2-ol

To a solution of 2-benzyloxyspiro[3.3]heptan-7-amine (270 mg, 1.24 mmol, 1 eq) in THF (10 mL) was added Pd/C (66.11 mg, 62.12 μmol, 10% purity, 0.05 eq). The suspension was degassed and purged with H₂ for 3 times. The mixture was stirred under H₂ (50 Psi) at 40° C. for 12 h. LCMS showed starting material was consumed and the desired mass was detected. The mixture was filtered, and concentrated under pressure to give compound 7-aminospiro[3.3]heptan-2-ol (110 mg, crude) as yellow oil.

LC-MS [ESI, M+1]: 128.3.

### Step 5: N-(2-hydroxyspiro[3.3]heptan-7-yl)-8-methyl-6-[(3R)-3-methyl-3-phenyl-pyrrolidin-1-yl]-[1,2,4]triazolo[1,5-a]pyridine-2-carboxamide (Compound 676)

To a solution of 8-methyl-6-[(3R)-3-methyl-3-phenyl-pyrrolidin-1-yl]-[1,2,4]triazolo[1,5-a]pyridine-2-carboxylic acid (100 mg, 297.28 μmol, 1 eq) and 7-aminospiro[3.3]heptan-2-ol (75.62 mg, 594.55 μmol, 2 eq) in DMF (1 mL) was added HATU (169.55 mg, 445.92 μmol, 1.5 eq) and DIEA (115.26 mg, 891.83 μmol, 155.34 μL, 3 eq). The mixture was stirred at 25° C. for 0.5 h. LCMS showed starting material was consumed and the desired mass was detected. The reaction mixture was quenched by H₂O (8 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.54) to give compound N-(2-hydroxyspiro[3.3]heptan-7-yl)-8-methyl-6-[(3R)-3-methyl-3-phenyl-pyrrolidin-1-yl]-[1,2,4]triazolo[1,5-a]pyridine-2-carboxamide (90 mg, 68% yield) as white solid.

LC-MS [ESI, M+1]: 446.3.

¹H NMR (400 MHz, CHLOROFORM-d) δ=7.66 (s, 1H), 7.50 (br d, J=8.4 Hz, 1H), 7.40-7.34 (m, 2H), 7.34-7.29 (m, 2H), 7.25 (br s, 1H), 7.01 (s, 1H), 4.55 (br d, J=8.4 Hz, 1H), 4.18 (br t, J=7.1 Hz, 1H), 3.61 (br d, J=8.8 Hz, 1H), 3.57-3.47 (m, 2H), 3.45-3.38 (m, 1H), 2.73 (br dd, J=6.2, 11.2 Hz, 1H), 2.64 (s, 4H), 2.45-2.35 (m, 1H), 2.32-2.21 (m, 2H), 1.95 (br dd, J=7.2, 11.6 Hz, 1H), 1.83 (br d, J=3.6 Hz, 4H), 1.47 (s, 3H).

### Step 6: ethyl 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetate

To a solution of N-(2-hydroxyspiro[3.3]heptan-7-yl)-8-methyl-6-[(3R)-3-methyl-3-phenyl-pyrrolidin-1-yl]-[1,2,4]triazolo[1,5-a]pyridine-2-carboxamide (40 mg, 89.78 μmol, 1 eq) in DCM (2 mL) was added Rhodium(II) acetate (3.97 mg, 8.98 μmol, 0.1 eq) and ethyl diazoacetate (20.85 mg, 179.55 μmol, 2 eq). The mixture was stirred at 25° C. for 2 h under N₂ atmosphere. LCMS showed starting material was consumed and the desired mass was detected. The reaction mixture was quenched by H₂O (5 mL), then extracted with DCM (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.61) to give compound ethyl 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetate (15 mg, 32% yield) as green gum.

LC-MS [ESI, M+1]: 532.4.

### Step 7: 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetic acid (Compound 677)

To a solution of ethyl 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetate (10 mg, 18.81 μmol, 1 eq) in THF (1 mL) was added TMSOK (4.83 mg, 37.62 μmol, 2 eq) and H₂O (0.3 mL). The mixture was stirred at 25° C. for 2 h. LCMS showed starting material was consumed and the desired mass was detected. The reaction was concentrated under reduced pressure to give a residue. The residue was purified by prep-HPLC purification (mobile phase: [water(FA)—ACN]; B %: 40%-70%, 10 min), the mixture was concentrated and lyophilized to give compound 2-[7-[[8-methyl-6-[(3R)-3-methyl-3-phenyl-pyrrolidin-1-yl]-[1,2,4]triazolo[1,5-a]pyridine-2-carbonyl]amino]spiro[3.3]heptan-2-yl]oxyacetic acid (1.98 mg, 21% yield) as a gray solid.

LC-MS [ESI, M+1]: 504.4.

¹H NMR (400 MHz, CHLOROFORM-d) δ=7.70 (br s, 1H), 7.63 (br d, J=7.2 Hz, 1H), 7.40-7.34 (m, 2H), 7.33-7.29 (m, 2H), 7.26-7.22 (m, 1H), 7.11 (br s, 1H), 4.53-4.38 (m, 1H), 4.01 (br d, J=7.2 Hz, 1H), 3.97 (s, 2H), 3.59 (br d, J=9.2 Hz, 1H), 3.55-3.47 (m, 2H), 3.41 (br d, J=3.2 Hz, 1H), 2.71-2.67 (m, 1H), 2.62 (br s, 3H), 2.36 (br s, 1H), 2.28 (br s, 3H), 2.02 (br dd, J=7.2, 11.2 Hz, 1H), 1.96-1.78 (m, 4H), 1.45 (s, 3H).

### Example 108

### 4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylic acid (Compound 678)

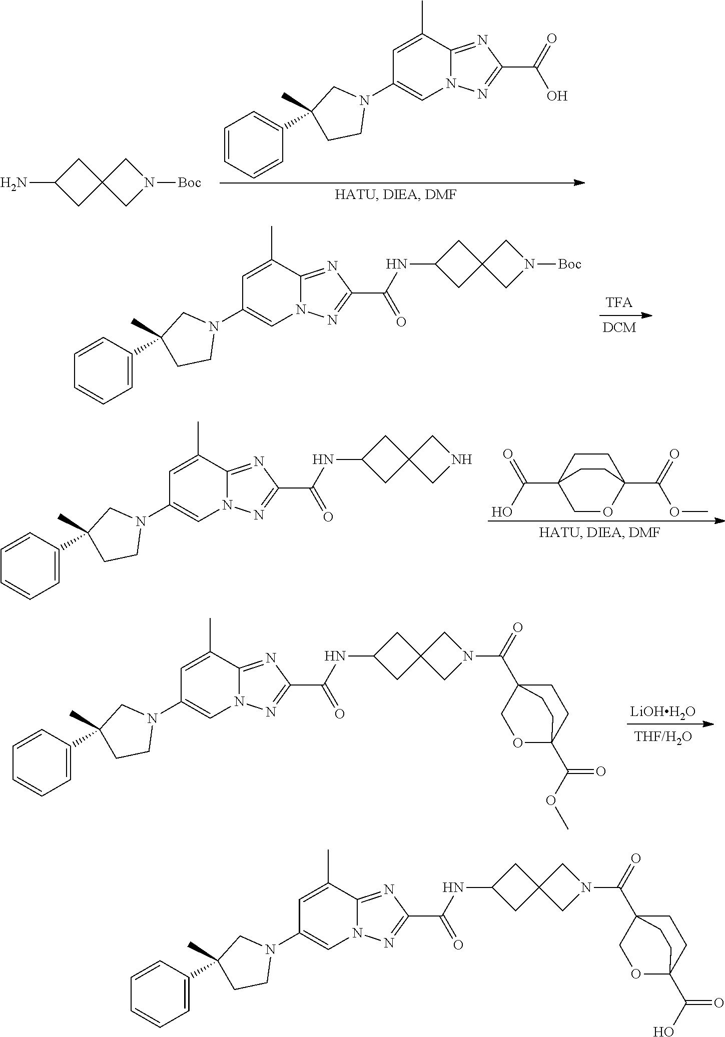

### Step 1: tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylat

To a solution of tert-butyl 6-amino-2-azaspiro[3.3]heptane-2-carboxylate (347.10 mg, 1.64 mmol, 1.1 eq) and 8-methyl-6-[(3R)-3-methyl-3-phenyl-pyrrolidin-1-yl]-[1,2,4]triazolo[1,5-a]pyridine-2-carboxylic acid (500 mg, 1.49 mmol, 1 eq) in DMF (5 mL) was added HATU (847.75 mg, 2.23 mmol, 1.5 eq) and DIEA (576.31 mg, 4.46 mmol, 776.70 μL, 3 eq) The mixture was stirred at 25° C. for 1 h. LCMS showed starting material was consumed and the desired mass was detected. The reaction was filtered and the filtered concentrated under reduced pressure to give a residue. The crude product was purified by reversed-phase HPLC (0.1% FA condition) to give compound tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylate (600 mg, 76.07% yield) as a white solid.

LC-MS [ESI, M+1]:531.8

¹H NMR (400 MHz, CHLOROFORM-d) δ=7.65 (d, J=1.6 Hz, 1H), 7.45 (br d, J=7.6 Hz, 1H), 7.41-7.34 (m, 2H), 7.33-7.30 (m, 2H), 7.27-7.22 (m, 1H), 7.01 (s, 1H), 4.65-4.48 (m, 1H), 4.00 (s, 2H), 3.90 (s, 2H), 3.60 (d, J=8.8 Hz, 1H), 3.57-3.45 (m, 2H), 3.41 (dt, J=3.6, 8.8 Hz, 1H), 2.71 (ddd, J=3.2, 7.6, 10.0 Hz, 2H), 2.63 (s, 3H), 2.39 (td, J=8.2, 12.2 Hz, 1H), 2.31-2.21 (m, 3H), 1.49-1.42 (m, 12H).

### Step 2: N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide

To a solution of tert-butyl 6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carboxylate (600 mg, 1.13 mmol, 1 eq) in DCM (7 mL) was added TFA (386.77 mg, 3.39 mmol, 251.97 μL, 3 eq). The mixture was stirred at 25° C. for 0.5 h. LCMS showed starting material was consumed and the desired mass was detected. The reaction was filtered and the filtered concentrated under reduced pressure to give a residue. The crude product was purified by reversed-phase HPLC (0.1% FA condition) to give compound N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide (600 mg, 97.45% yield, TFA) as a white solid.

LC-MS [ESI, M+1]:431.2

¹H NMR (400 MHz, DMSO-d₆) δ=8.76-8.66 (m, 2H), 7.85 (d, J=1.6 Hz, 1H), 7.43-7.29 (m, 5H), 7.28-7.18 (m, 1H), 4.44-4.25 (m, 1H), 4.05 (br t, J=6.0 Hz, 2H), 3.92 (br t, J=6.2 Hz, 2H), 3.61-3.57 (m, 1H), 3.55-3.44 (m, 2H), 3.42-3.30 (m, 1H), 2.66-2.56 (m, 2H), 2.55 (s, 3H), 2.45-2.36 (m, 2H), 2.29-2.23 (m, 2H), 1.35 (s, 3H).

### Step 3:methyl 4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylate

To a solution of N-(2-azaspiro[3.3]heptan-6-yl)-8-methyl-6-[(3R)-3-methyl-3-phenyl-pyrrolidin-1-yl]-[1,2,4]triazolo[1,5-a]pyridine-2-carboxamide (25 mg, 45.91 μmol, 1 eq, TFA) and 1-methoxycarbonyl-2-oxabicyclo[2.2.2]octane-4-carboxylic acid (14.75 mg, 68.86 μmol, 1.5 eq) in DMF (1 mL) was added HATU (26.18 mg, 68.86 μmol, 1.5 eq) and DIEA (17.80 mg, 137.72 μmol, 23.99 μL, 3 eq). The mixture was stirred at 25° C. for 1 h. LCMS showed starting material was consumed and the desired mass was detected. The reaction was filtered and the filtered concentrated under reduced pressure to give a residue. The crude product was purified by reversed-phase HPLC 0.1% FA condition) to give compound methyl 4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylate (25 mg, 86.89% yield) as a white solid.

LC-MS [ESI, M+1]: 627.5.

### Step 4:4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylic acid

To a solution of methyl 4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylate (20 mg, 31.91 μmol, 1 eq) in THF (1 mL) and Water (0.5 mL) was added LiOH·H₂O (4.02 mg, 95.73 μmol, 3 eq). The mixture was stirred at 25° C. for 0.5 h. LCMS showed starting material was consumed and the desired mass was detected. The reaction was filtered and the filtered concentrated under reduced pressure to give a residue. The residue was purified by prep-HPLC (column: CD25-XPT PHS C18 150*25*10 um;mobile phase: [water(FA)—ACN]; gradient:35%-65% B over 10 min). The result solution was lyophilized to give compound 4-[6-[[8-methyl-6-[(3R)-3-methyl-3-phenyl-pyrrolidin-1-yl]-[1,2,4]triazolo[1,5-a]pyridine-2-carbonyl]amino]-2-azaspiro[3.3]heptane-2-carbonyl]-2-oxabicyclo[2.2.2]octane-1-carboxylic acid (11.75 mg, 58.99% yield, 98.158% purity) as a white solid.

LC-MS [ESI, M+1]: 613.2.

¹H NMR (400 MHz, CHLOROFORM-d) δ=7.71-7.42 (m, 2H), 7.40-7.28 (m, 4H), 7.27-7.23 (m, 1H), 7.01 (s, 1H), 4.67-4.48 (m, 1H), 4.48-4.26 (m, 2H), 4.25-3.85 (m, 4H), 3.60 (br d, J=8.8 Hz, 1H), 3.57-3.45 (m, 2H), 3.41 (dt, J=3.2, 8.8 Hz, 1H), 2.78-2.68 (m, 2H), 2.62 (s, 3H), 2.45-2.21 (m, 5H), 2.17-1.97 (m, 7H), 1.46 (s, 3H).

| Compound | | |
|---|---|---|
| Number | Synthesis | LC/MS |
| 678 | Example 108 | m/z 613.2 |
| | | [M + H]⁺ |
| 679 | Similar to | m/z 575.1 |
| | Example 108 | [M + H]⁺ |
| 680 | Similar to | m/z 571.2 |
| | Example 108 | [M + H]⁺ |
| 681 | Similar to | m/z 569.4 |
| | Example 108 | [M + H]⁺ |

### Example 109

### 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylic acid (Compound 682)

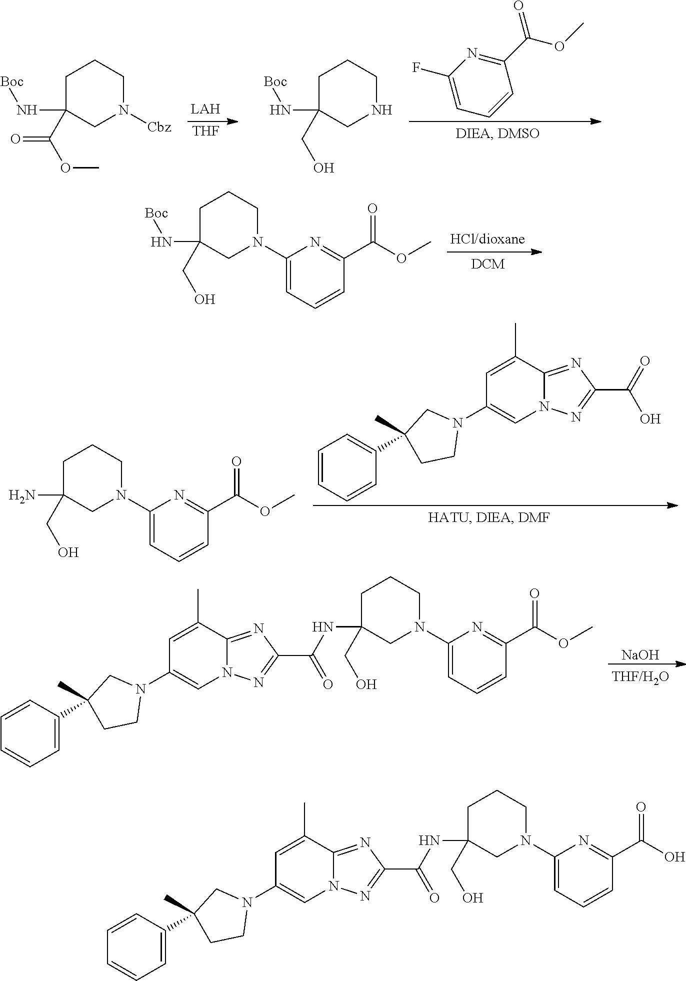

### Step 1: tert-butyl N-[3-(hydroxymethyl)-3-piperidyl]carbamate

To a solution of O1-benzyl O3-methyl 3-(tert-butoxycarbonylamino) piperidine-1, 3-dicarboxylate (600 mg, 1.53 mmol, 1 eq) in THF (6 mL) was added LAH (2.5 M, 1.83 mL, 3 eq) at 0° C. The mixture was stirred at 0° C. for 10 min. LCMS showed starting material was consumed and the desired mass was detected. The reaction mixture was quenched by KNaC₄H₁₂O₁₀·4H₂O (2.5 g) at 0° C. for 0.5 h, the mixture was filtered and concentrated to give compound tert-butyl N-[3-(hydroxymethyl)-3-piperidyl]carbamate (380 mg, 54% yield) as colorless oil.

### Step 2: methyl 6-[3-(tert-butoxycarbonylamino)-3-(hydroxymethyl)-1-piperidyl]pyridine-2-carboxylate

To a solution of tert-butyl N-[3-(hydroxymethyl)-3-piperidyl]carbamate (380 mg, 825.00 μmol, 1 eq) in DMSO (3 mL) was added DIEA (319.87 mg, 2.47 mmol, 431.10 μL, 3 eq) and methyl 6-fluoropyridine-2-carboxylate (63.99 mg, 412.50 μmol, 0.5 eq). The mixture was stirred at 120° C. for 1 h. LCMS showed starting material was consumed and the desired mass was detected. The reaction mixture was quenched by addition water (15 mL), and then diluted with EtOAc (15 mL) and extracted with EtOAc (15 mL×3). The combined organic layers were dried over Na₂SO₄, filtered and concentrated under reduced pressure to give a residue. The crude product was purified by reversed-phase-HPLC (0.1% FA condition) to give compound methyl 6-[3-(tert-butoxycarbonylamino)-3-(hydroxymethyl)-1-piperidyl]pyridine-2-carboxylate (80 mg, 27% yield) as yellow solid.

LC-MS [ESI, M+1]: 366.2.

¹H NMR (400 MHz, CHLOROFORM-d) δ=7.56 (dd, J=7.6, 8.4 Hz, 1H), 7.41-7.34 (m, 1H), 6.86 (d, J=8.8 Hz, 1H), 4.24 (br d, J=13.2 Hz, 1H), 3.95 (s, 3H), 3.69 (br d, J=13.2 Hz, 2H), 3.60 (d, J=12.0 Hz, 1H), 3.51-3.41 (m, 1H), 3.30 (d, J=12.0 Hz, 1H), 1.77-1.66 (m, 2H), 1.62-1.54 (m, 2H), 1.44 (s, 9H)

### Step 3: methyl 6-(3-amino-3-(hydroxymethyl)piperidin-1-yl)picolinate

To a solution of methyl 6-[3-(tert-butoxycarbonylamino)-3-(hydroxymethyl)-1-piperidyl]pyridine-2-carboxylate (40 mg, 109.46 μmol, 1 eq) in DCM (1 mL) was added HCl/dioxane (1 mL). The mixture was stirred at 25° C. for 0.5 h. LCMS showed starting material was consumed and the desired mass was detected. The mixture was concentrated to give compound methyl 6-[3-amino-3-(hydroxymethyl)-1-piperidyl]pyridine-2-carboxylate (29 mg, 79% yield, HCl) as yellow solid.

LC-MS [ESI, M+1]: 266.2.

### Step 4: methyl 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylate

To a solution of methyl 6-[3-amino-3-(hydroxymethyl)-1-piperidyl]pyridine-2-carboxylate (27.60 mg, 104.05 μmol, 1 eq) and 8-methyl-6-[(3R)-3-methyl-3-phenyl-pyrrolidin-1-yl]-[1,2,4]triazolo[1,5-a]pyridine-2-carboxylic acid (35 mg, 104.05 μmol, 1 eq) in DMF (1 mL) was added DIEA (40.34 mg, 312.14 μmol, 54.37 μL, 3 eq) and HATU (59.34 mg, 156.07 μmol, 1.5 eq). The mixture was stirred at 25° C. for 10 min. LCMS showed starting material was consumed and the desired mass was detected. The reaction mixture was quenched by addition water (5 mL), and then diluted with EtOAc (3 mL) and extracted with EtOAc (3 mL×3). The combined organic layers were dried over Na₂SO₄, filtered and concentrated under reduced pressure to give compound methyl 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylate (60 mg, 76% yield) as yellow solid.

LC-MS [ESI, M+1]: 584.4.

### Step 5: 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylic acid

To a solution of methyl 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylate (60 mg, 102.80 μmol, 1 eq) in THF (1 mL) and H₂O (1 mL) was added NaOH (6.17 mg, 154.19 μmol, 1.5 eq). The mixture was stirred at 25° C. for 0.5 h. LCMS showed starting material was consumed and one main peak with desired mass was detected. The reaction was quenched by FA (0.1 mL), the mixture was partitioned between EtOAc (5 mL×3) and water (10 mL), the combined organic phase was dried over anhydrous sodium sulfate, filtered and concentrated to give a residue. The crude product was purified by reversed-phase HPLC (column: CD01-Phenomenex luna C18 150*25 mm*10 um;mobile phase: [H₂O (0.225% FA)—ACN]; gradient:35%-65% B over 11.0 min) to give compound 6-[3-(hydroxymethyl)-3-[[8-methyl-6-[(3R)-3-methyl-3-phenyl-pyrrolidin-1-yl]-[1,2,4]triazolo[1,5-a]pyridine-2-carbonyl]amino]-1-piperidyl]pyridine-2-carboxylic acid (15.39 mg, 26% yield) as white solid.

LC-MS [ESI, M+1]: 570.3.

¹H NMR (400 MHz, CHLOROFORM-d) δ=7.73 (s, 1H), 7.62 (br t, J=8.0 Hz, 1H), 7.57 (s, 1H), 7.43 (d, J=7.2 Hz, 1H), 7.36 (br d, J=7.2 Hz, 2H), 7.34-7.29 (m, 2H), 7.24 (s, 1H), 7.01 (br d, J=8.8 Hz, 1H), 6.95 (s, 1H), 4.61 (br d, J=14.0 Hz, 1H), 4.11-3.96 (m, 2H), 3.92-3.84 (m, 1H), 3.59 (br d, J=8.8 Hz, 1H), 3.55-3.45 (m, 2H), 3.44-3.35 (m, 2H), 3.33-3.23 (m, 1H), 2.53 (s, 3H), 2.46-2.32 (m, 2H), 2.30-2.22 (m, 1H), 1.84-1.76 (m, 3H), 1.46 (s, 3H).

| Compound | | |
|---|---|---|
| Number | Synthesis | LC/MS |
| 683 | Similar to | m/z 554.5 |
| | Example 109 | [M + H]⁺ |
| 684 | Similar to | m/z 542.3 |
| | Example 109 | [M + H]⁺ |

### Example 110

### 6-(7-(8-methyl-6-((R)-3-methyl-3-phenylpyrrolidin-1-yl)-[1,2,4]triazolo[1,5-a]pyridine-2-carboxamido)-3-azabicyclo[4.1.0]heptan-3-yl)picolinic acid (Compound 685)

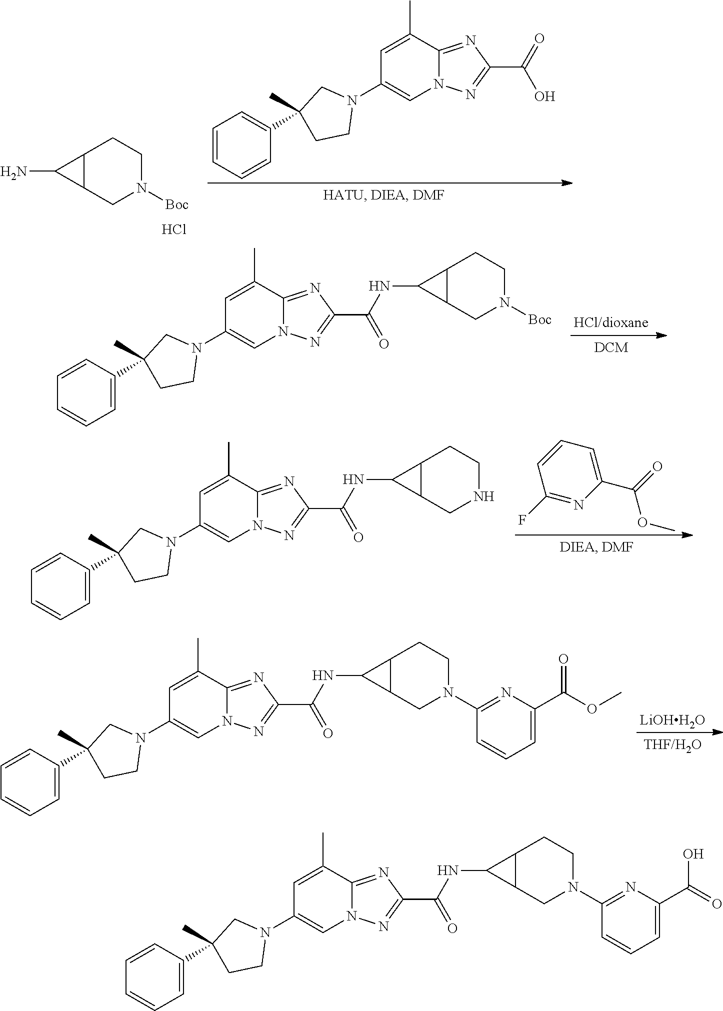

### Step 1: tert-butyl 7-(8-methyl-6-((R)-3-methyl-3-phenylpyrrolidin-1-yl)-[1,2,4]triazolo[1,5-a]pyridine-2-carboxamido)-3-azabicyclo[4.1.0]heptane-3-carboxylate

To a solution of tert-butyl 7-amino-3-azabicyclo[4.1.0]heptane-3-carboxylate (75.73 mg, 356.73 μmol, 1.5 eq) and 8-methyl-6-[rac-(3R)-3-methyl-3-phenyl-pyrrolidin-1-yl]-[1,2,4]triazolo[1,5-a]pyridine-2-carboxylic acid (80 mg, 237.82 μmol, 1 eq) in DMF (2 mL) was added HATU (180.85 mg, 475.64 μmol, 2 eq) and DIEA (92.21 mg, 713.46 μmol, 124.27 μL, 3 eq). The mixture was stirred at 25° C. for 15 min. LCMS showed starting material was consumed and the desired mass was detected. The reaction mixture was added H₂O (10 ml) and then was extracted with EtOAc (10 mL×3), The combined organic layers were washed with brine (30 mL×3), dried over anhydrous Na₂SO₄, filtered and dried to give compound tert-butyl 7-(8-methyl-6-((R)-3-methyl-3-phenylpyrrolidin-1-yl)-[1,2,4]triazolo[1,5-a]pyridine-2-carboxamido)-3-azabicyclo[4.1.0]heptane-3-carboxylate (90 mg, 169.60 μmol, 71.31% yield) as a white gum.

LC-MS [ESI, M+1]: 531.4.

### Step 2: N-(3-azabicyclo[4.1.0]heptan-7-yl)-8-methyl-6-((R)-3-methyl-3-phenylpyrrolidin-1-yl)-[1,2,4]triazolo[1,5-a]pyridine-2-carboxamide

To a solution of tert-butyl 7-[[8-methyl-6-[rac-(3R)-3-methyl-3-phenyl-pyrrolidin-1-yl]-[1,2,4]triazolo[1,5-a]pyridine2-carbonyl]amino]-3-azabicyclo[4.1.0]heptane-3-carboxylate (90 mg, 169.60 μmol, 1 eq) in DCM (2 mL) was added TFA (307.00 mg, 2.69 mmol, 0.2 mL, 15.88 eq). The mixture was stirred at 25° C. for 15 min. LCMS showed starting material was consumed and the desired mass was detected. The reaction mixture was concentrated under the reduced pressure to give compound N-(3-azabicyclo[4.1.0]heptan-7-yl)-8-methyl-6-((R)-3-methyl-3-phenylpyrrolidin-1-yl)-[1,2,4]triazolo[1,5-a]pyridine-2-carboxamide (78 mg, crude, TFA) as a yellow gum.

LC-MS [ESI, M+1]: 431.2.

### Step 3: methyl 6-(7-(8-methyl-6-((R)-3-methyl-3-phenylpyrrolidin-1-yl)-[1,2,4]triazolo[1,5-a]pyridine-2-carboxamido)-3-azabicyclo[4.1.0]heptan-3-yl)picolinate

To a solution of N-(3-azabicyclo[4.1.0]heptan-7-yl)-8-methyl-6-[rac-(3R)-3-methyl-3-phenyl-pyrrolidin-1-yl]-[1,2,4]triazolo[1,5-a]pyridine-2-carboxamide (70 mg, 149.89 μmol, 1 eq, HCl) and methyl 6-fluoropyridine-2-carboxylate (23.25 mg, 149.89 μmol, 1 eq) in DMSO (3 mL) was added DIEA (96.86 mg, 749.46 μmol, 130.54 μL, 5 eq). The mixture was stirred at 100° C. for 1 hr. LCMS showed starting material was consumed and the desired mass was detected. The reaction mixture was added H₂O (10 ml) and then was extracted with EtOAc (10 mL×3), The combined organic layers were washed with brine (30 mL×3), dried over anhydrous Na₂SO₄, filtered and dried to give a residue. The residue was purified by prep-TLC (SiO₂, PE:EtOAc=0:1, Rf=0.5) and eluted with 60 mL (10% methanol in DCM) to give compound methyl 6-(7-(8-methyl-6-((R)-3-methyl-3-phenylpyrrolidin-1-yl)-[1,2,4]triazolo[1,5-a]pyridine-2-carboxamido)-3-azabicyclo[4.1.0]heptan-3-yl)picolinate (30 mg, 53.04 μmol, 35.38% yield) as a yellow gum.

LC-MS [ESI, M+1]: 566.2

### Step 4: 6-(7-(8-methyl-6-((R)-3-methyl-3-phenylpyrrolidin-1-yl)-[1,2,4]triazolo[1,5-a]pyridine-2-carboxamido)-3-azabicyclo[4.1.0]heptan-3-yl)picolinic acid

To a solution of methyl 6-[7-[[8-methyl-6-[rac-(3R)-3-methyl-3-phenyl-pyrrolidin-1-yl]-[1,2,4]triazolo[1,5-a]pyridine-2-carbonyl]amino]-3-azabicyclo[4.1.0]heptan-3-yl]pyridine-2-carboxylate (25 mg, 44.20 μmol, 1 eq) in THF (1 mL) and H₂O (0.2 mL) was added LiOH·H₂O (7.42 mg, 176.78 μmol, 4 eq). The mixture was stirred at 25° C. for 15 min. LCMS showed starting material was consumed and the desired mass was detected. The mixture was concentrated to give residue, the residue was adjusted PH around 3-4 by FA, solid was precipitate out, and the mixture was filtered and the solid was concentrated under reduced pressure to give a residue. The residue was purified by prep-HPLC (column: Phenomenex luna C18 150*25 mm*10 um; mobile phase: [water(FA)—ACN]; gradient:35%-65% B over 8 min) and lyophilized to give compound 6-(7-(8-methyl-6-((R)-3-methyl-3-phenylpyrrolidin-1-yl)-[1,2,4]triazolo[1,5-a]pyridine-2-carboxamido)-3-azabicyclo[4.1.0]heptan-3-yl)picolinic acid (10.2 mg, 18.20 μmol, 41.18% yield, 98.430% purity) as a white solid.

LC-MS [ESI, M+1]:552.2

¹H NMR (CHLOROFORM-d, 400 MHz): δ=7.62-7.71 (m, 2H), 7.49 (d, J=7.2 Hz, 1H), 7.41 (s, 1H), 7.36 (d, J=7.2 Hz, 2H), 7.29-7.34 (m, 2H), 7.00 (s, 1H), 6.86 (d, J=8.4 Hz, 1H), 4.16 (d, J=13.2 Hz, 1H), 3.80-3.90 (m, 1H), 3.55-3.64 (m, 2H), 3.46-3.54 (m, 2H), 3.32-3.44 (m, 2H), 2.85 (d, J=3.2 Hz, 1H), 2.61 (s, 3.1H), 2.37 (s, 1.2H), 2.15-2.32 (m, 3H), 2.09 (dd, J=14.4, 7.4 Hz, 2H), 1.47 (s, 3.0H).

| Compound | | |
|---|---|---|
| Number | Synthesis | LC/MS |
| 686 | Similar to | m/z 556.2 |
| | Example 110 | [M + H]⁺ |
| 687 | Similar to | m/z 582.4 |
| | Example 110 | [M + H]⁺ |
| 688 | Similar to | m/z 566.1 |
| | Example 110 | [M + H]⁺ |
| 689 | Similar to | m/z 540.3 |
| | Example 110 | [M + H]⁺ |

### Example 111

### 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo[3.2.1]octan-3-yl]pyridine-2-carboxylic acid (Compound 690)

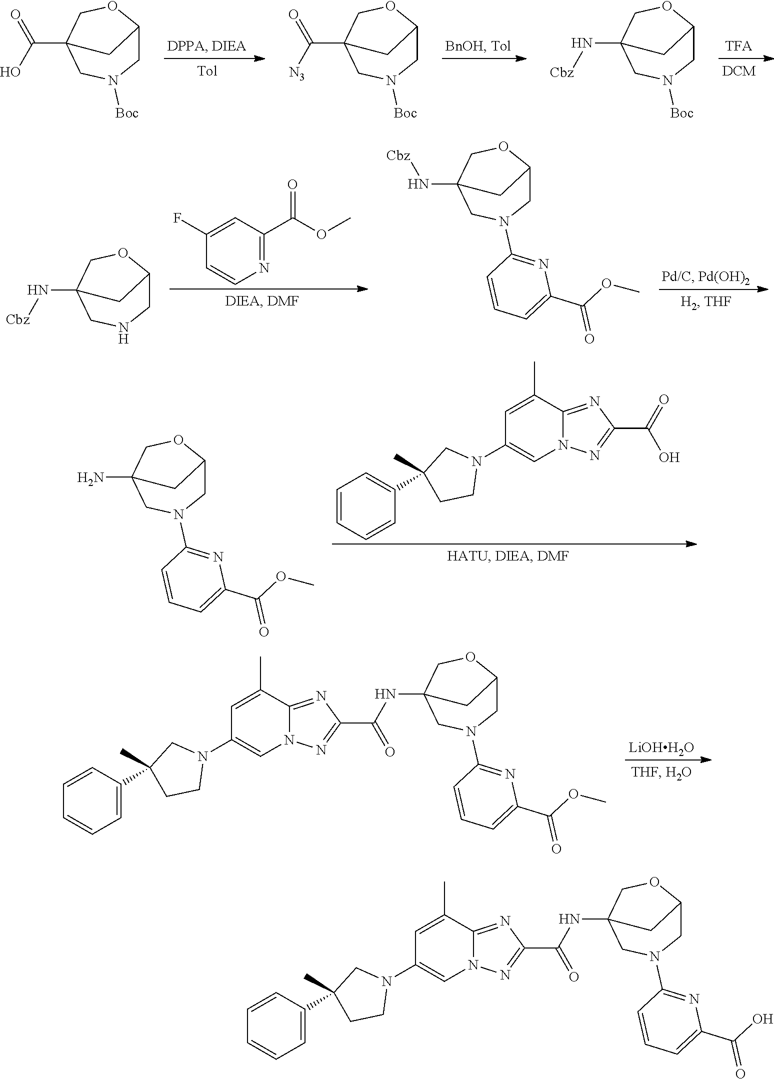

### Step 1: tert-butyl 1-carbonazidoyl-6-oxa-3-azabicyclo [3.2.1]octane-3-carboxylate

To a mixture of 3-tert-butoxycarbonyl-6-oxa-3-azabicyclo [3.2.1]octane-1-carboxylic acid (400 mg, 1.55 mmol, 1 eq) and DIEA (602.79 mg, 4.66 mmol, 812.39 μL, 3 eq) in Tol. (5 mL) was added DPPA (641.79 mg, 2.33 mmol, 503.36 μL, 1.5 eq), and then the mixture was stirred at 25° C. for 6 h. LCMS showed starting material was consumed and one main peak with desired mass was detected. The reaction mixture was concentrated under the reduced pressure to give the product. The reaction was added water (10 mL) and then extracted with DCM (10 mL×3). The combined organic phase was washed with brine (5 mL), dried with anhydrous Na₂SO₄, filtered and concentrated under pressure to give the product was purified by reversed-phase HPLC (0.1% FA condition) to give compound tert-butyl 1-carbonazidoyl-6-oxa-3-azabicyclo[3.2.1]octane-3-carboxylate (430 mg, crude) as colorless oil.

LC-MS [ESI, M-28+1]: 255.1

### Step 2: tert-butyl 1-(((benzyloxy)carbonyl)amino)-6-oxa-3-azabicyclo[3.2.1]octane-3-carboxylate

To a solution of tert-butyl 1-carbonazidoyl-6-oxa-3-azabicyclo[3.2.1]octane-3-carboxylate (430 mg, 1.52 mmol, 1 eq) in Tol. (2 mL) was added BnOH (1.65 g, 15.23 mmol, 1.58 mL, 10 eq). The mixture was stirred at 90° C. for 2 h. LCMS showed starting material was consumed and one main peak with desired mass was detected. The reaction mixture was concentrated under the reduced pressure to give the product. The reaction was added water (10 mL) and then extracted with ethyl acetate (10 mL×3).The combined organic phase was washed with brine (5 mL), dried with anhydrous Na₂SO₄, filtered, and concentrated under pressure to give the product. The residue was purified by column chromatography (SiO₂, PE:EtOAc=7:3, Rf=0.16) to give compound tert-butyl 1-(benzyloxycarbonylamino)-6-oxa-3-azabicyclo [3.2.1]octane-3-carboxylate (414 mg, 434.08 μmol, 28.50% yield, 38% purity) as colorless oil.

LC-MS [ESI, M-56]: 307.38

¹H NMR (400 MHz, CHLOROFORM-d) δ=7.28 (br s, 5H), 5.71-5.42 (m, 1H), 5.01 (br s, 2H), 4.23 (br s, 1H), 4.14 (br s, 1H), 4.00-3.90 (m, 1H), 3.84 (br d, J=12.4 Hz, 1H), 2.98 (br d, J=11.2 Hz, 1H), 2.78-2.59 (m, 2H), 2.31-2.07 (m, 2H), 1.39 (br s, 9H)

### Step 3: benzyl N-(6-oxa-3-azabicyclo[3.2.1]octan-1-yl)carbamate

To a mixture after t-butyl 1-(benzyloxycarbonylamino)-6-oxa-3-azabicyclo [3.2.1]octane-3-carboxylate (400 mg, 1.10 mmol, 1 eq) in DCM (2 mL) was added TFA (956.51 mg, 8.39 mmol, 623.13 μL, 7.60 eq), and then the mixture was stirred at 25° C. for 2 h. LCMS showed starting material was consumed and the desired mass was detected. The reaction was concentrated under reduced pressure to give compound benzyl N-(6-oxa-3-azabicyclo [3.2.1]octan-1-yl)carbamate (665 mg, crude, TFA) as yellow oil.

LC-MS [ESI, M+1]: 263.2

### Step 4: methyl 6-[1-(benzyloxycarbonylamino)-6-oxa-3-azabicyclo[3.2.1]octan-3-yl]pyridine-2-carboxylate

To a mixture of methyl 4-fluoropyridine-2-carboxylate (267.94 mg, 1.73 mmol, 1 eq) and benzyl N-(6-oxa-3-azabicyclo [3.2.1]octan-1-yl) carbamate (650 mg, 1.73 mmol, 1 eq, TFA) in DMF (5 mL) was added DIEA (669.68 mg, 5.18 mmol, 902.53 μL, 3 eq), and then the mixture was stirred at 100° C. for 2 h. LCMS showed starting material was consumed and the desired mass was detected. The reaction mixture was diluted with water (10 mL) and extracted with ethyl acetate (10 mL×3). The combined organic layers was washed with saturated brine. It was dried over Na₂SO₄, filtered and concentrated under reduced pressure to give a residue. The residue was purified by column chromatography (SiO₂, PE:EtOAc=3:1, Rf=0.1) methyl 6-[1-(benzyloxycarbonylamino)-6-oxa-3-azabicyclo [3.2.1]octan-3-yl]pyridine-2-carboxylate (140 mg, 352.27 μmol, 20.40% yield, 100% purity) as yellow liquid.

LC-MS [ESI, M+1]: 398.3

### Step 5: methyl 6-(1-amino-6-oxa-3-azabicyclo[3.2.1]octan-3-yl)pyridine-2-carboxylate

To a solution of methyl 6-[1-(benzyloxycarbonylamino)-6-oxa-3-azabicyclo [3.2.1]octan-3-yl]pyridine-2-carboxylate (140 mg, 352.27 μmol, 1 eq) in THF (5 mL) was added Pd/C (39.27 mg, 352.27 μmol, 10% purity, 1 eq) and Pd(OH)₂ (51.83 mg, 352.27 μmol, 10% purity, 1 eq) under N₂. The suspension was degassed under vacuum and purged with H₂ several times. The mixture was stirred under H₂ (15 psi) at 25° C. for 2 h. LCMS showed starting material was consumed and one main peak with desired mass was detected. The reaction mixture was concentrated under the reduced pressure to give the compound methyl 6-(1-amino-6-oxa-3-azabicyclo[3.2.1]octan-3-yl)pyridine-2-carboxylate (90 mg, crude) as a yellow oil.

LC-MS [ESI, M+1]: 264.2

### Step 6: methyl 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo[3.2.1]octan-3-yl]pyridine-2-carboxylate

To a solution of 8-methyl-6-[rac-(3R)-3-methyl-3-phenyl-pyrrolidin-1-yl]-[1,2,4]triazolo[1,5-a]pyridine-2-carboxylic acid (30 mg, 89.18 μmol, 1 eq) in DMF (2 mL) was added HATU (40.69 mg, 107.02 μmol, 1.2 eq) and DIEA (34.58 mg, 267.55 μmol, 46.60 μL, 3 eq). And then added methyl 6-(1-amino-6-oxa-3-azabicyclo [3.2.1]octan-3-yl) pyridine-2-carboxylate (28.18 mg, 107.02 μmol, 1.2 eq). The mixture was stirred at 25° C. for 0.15 h. LCMS showed starting material was consumed and the desired mass was detected. The reaction mixture was diluted with H₂O (10 mL) and extracted with ethyl acetate (10 mL×3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water(FA)—ACN]; gradient:22%-52% B over 10 min) to give compound methyl 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo [3.2.1]octan-3-yl]pyridine-2-carboxylate (25 mg, 34.38 μmol, 38.55% yield, 80% purity) as yellow solid.

LC-MS [ESI, M+1]: 582.3

### Step 7: 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo[3.2.1]octan-3-yl]pyridine-2-carboxylic acid

To a mixture of methyl 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo[3.2.1]octan-3-yl]pyridine-2-carboxylate (25 mg, 42.98 μmol, 1 eq) and LiOH·H₂O (7.21 mg, 171.92 μmol, 4 eq) in THF (2 mL) was added H₂O (0.5 mL), and then the mixture was stirred at 25° C. for 0.15 h. LCMS showed starting material was consumed and the desired mass was detected. The reaction was concentrated under reduced pressure to give a residue. The residue was purified by prep-HPLC (column: Phenomenex luna C18 150*25 mm*10 um; mobile phase: [water(FA)—ACN]; gradient:25%-55% B over 15 min) to give compound 6-[1-[[8-methyl-6-[rac-(3R)-3-methyl-3-phenyl-pyrrolidin-1-yl]-[1,2,4]triazolo[1,5-a]pyridine-2-carbonyl]amino]-6-oxa-3-azabicyclo[3.2.1]octan-3-yl]pyridine-2-carboxylic acid (4.5 mg, 7.93 μmol, 18.44% yield, 100% purity) as white solid.

LC-MS [ESI, M+1]: 568.2

¹H NMR (400 MHz, CHLOROFORM-d) δ=8.37-8.11 (m, 1H), 8.01-7.74 (m, 1H), 7.69-7.47 (m, 2H), 7.36-7.20 (m, 5H), 6.93 (br d, J=2.8 Hz, 1H), 6.86-6.64 (m, 1H), 4.70-4.31 (m, 3H), 4.05-3.80 (m, 2H), 3.60-3.28 (m, 5H), 3.24-3.11 (m, 1H), 2.50-2.40 (m, 3H), 2.35 (br d, J=9.2 Hz, 1H), 2.30 (br d, J=2.8 Hz, 2H), 2.19 (br d, J=2.8 Hz, 1H), 1.39 (s, 3H).

### Example 112

### (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinic acid (Compound 691)

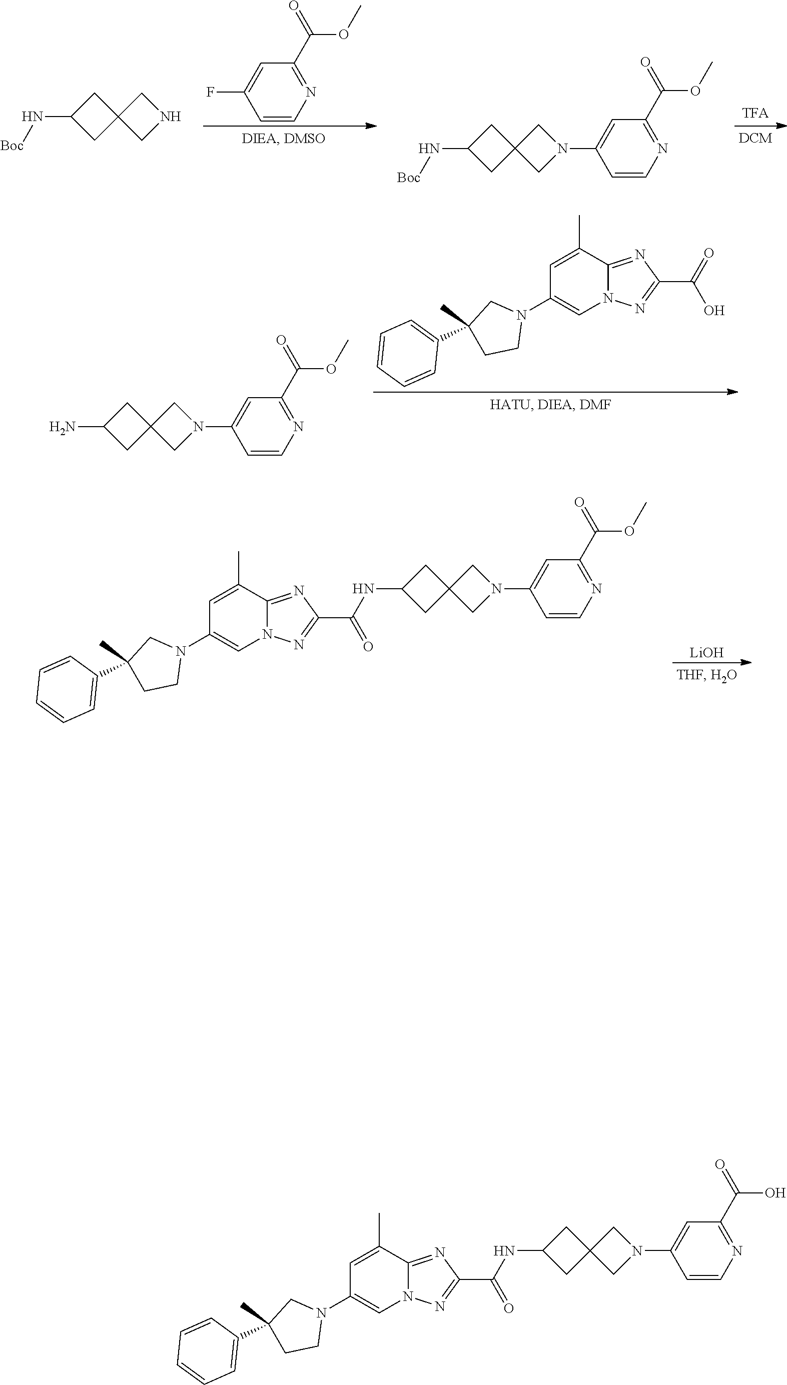

### Step 1: methyl 4-(6-((tert-butoxycarbonyl)amino)-2-azaspiro[3.3]heptan-2-yl)picolinate

To a solution of tert-butyl N-(2-azaspiro[3.3]heptan-6-yl)carbamate (300 mg, 1.41 mmol, 1 eq) and methyl 4-fluoropyridine-2-carboxylate (175.38 mg, 1.13 mmol, 0.8 eq) in DMSO (3 mL) was added DIEA (547.92 mg, 4.24 mmol, 738.43 μL, 3 eq). The mixture was stirred at 100° C. for 12 h. LCMS showed starting material was consumed and one main peak with desired mass was detected. The reaction mixture was added H₂O (5 ml) and then was extracted with EtOAc (5 mL×3), the combined organic layers were washed with brine (5 mL×3), dried over anhydrous Na₂SO₄, filtered and dried to give a residue. The residue was purified by prep-TLC (PE:EtOAc=0:1, Rf=0.1) to give compound methyl 4-(6-((tert-butoxycarbonyl)amino)-2-azaspiro[3.3]heptan-2-yl)picolinate (430 mg, 1.24 mmol, 87.59% yield) as a yellow solid.

LC-MS [ESI, M+1]: 348.2

¹H NMR (400 MHz, CHLOROFORM-d) δ=7.74-7.47 (m, 4H), 7.39-7.14 (m, 10H), 6.93 (s, 1H), 4.35 (s, 2H), 4.16 (t, J=6.4 Hz, 1H), 4.08-3.94 (m, 1H), 3.80-3.64 (m, 1H), 3.62-3.49 (m, 2H), 3.48-3.38 (m, 2H), 3.33 (dd, J=5.2, 8.8 Hz, 1H), 2.96-2.78 (m, 1H), 2.69-2.47 (m, 4H), 2.42-2.25 (m, 4H), 2.23-2.13 (m, 1H), 1.39 (s, 3H).

### Step 2: methyl 4-(6-amino-2-azaspiro[3.3]heptan-2-yl)picolinate

To a solution of methyl methyl 4-(6-((tert-butoxycarbonyl)amino)-2-azaspiro[3.3]heptan-2-yl)picolinate (430 mg, 1.24 mmol, 1 eq) in DCM (3 mL) was added TFA (141.13 mg, 1.24 mmol, 91.94 μL, 1 eq). The mixture was stirred at 25° C. for 2 h. LCMS showed starting material was consumed and one main peak with desired mass was detected. The mixture was concentrated in vacuo to give compound methyl 4-(6-amino-2-azaspiro[3.3]heptan-2-yl)picolinate (74 mg, crude, TFA) as a black brown gum.

LC-MS [ESI, M+1]: 248.2

### Step 3: methyl (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinate

To a solution of methyl 4-(6-amino-2-azaspiro[3.3]heptan-2-yl)picolinate (40 mg, 161.75 μmol, 1 eq) and 8-methyl-6-[(3R)-3-methyl-3-phenyl-pyrrolidin-1-yl]-[1,2,4]triazolo[1,5-a]pyridine-2-carboxylic acid (54.41 mg, 161.75 μmol, 1 eq) in DMF (3 mL) was added HATU (123.01 mg, 323.50 μmol, 2 eq) and DIEA (62.72 mg, 485.26 μmol, 84.52 μL, 3 eq). The mixture was stirred at 25° C. for 1 h. LCMS showed starting material was consumed and one main peak with desired mass was detected. The reaction mixture was added H₂O (5 ml) and then was extracted with EtOAc (5 mL×3), The combined organic layers were washed with brine (5 mL×3), dried over anhydrous Na₂SO₄, filtered and dried to give a residue. The residue was purified by prep-TLC (SiO₂, DCM:MeOH=10:1, Rf=0.5) and eluted with 60 mL (10% methanol in DCM) to give compound methyl (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinate (20 mg, 35.36 μmol, 21.86% yield) as a yellow solid.

LC-MS [ESI, M+1]: 566.3.

1H NMR (CHLOROFORM-d, 400 MHz): δ=8.24 (d, J=5.6 Hz, 1H), 7.59 (s, 1.0H), 7.48 (d, J=8.0 Hz, 1H), 7.31 (br d, J=7.2 Hz, 2H), 7.16-7.26 (m, 3.0H), 7.04 (d, J=2.4 Hz, 1H), 6.96 (s, 1H), 6.36 (dd, J=5.8, 2.4 Hz, 1H), 4.46-4.67 (m, 1H), 4.09 (s, 2H), 4.01 (s, 2H), 3.92 (s, 3.0H), 3.55 (d, J=8.8 Hz, 1H), 3.47 (d, J=8.0 Hz, 1H), 3.42 (s, 1H), 3.36 (td, J=8.8, 3.3 Hz, 1H), 2.70-2.82 (m, 2H), 2.57 (s, 3H), 2.23 (dd, J=7.2, 3.7 Hz, 4H), 1.41 (s, 3H).

### Step 4: (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinic acid

To a solution of methyl (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinate (20 mg, 35.36 μmol, 1 eq) in THF (0.5 mL) and H₂O (0.3 mL) was added LiOH·H₂O (4.45 mg, 106.07 μmol, 3 eq). The mixture was stirred at 25° C. for 1 h. LCMS showed starting material was consumed and one main peak with desired mass was detected. The reaction mixture was concentrated under the reduced pressure to give the product. The residue was purified by prep-HPLC (column: Welch Xtimate C18 150*25 mm*5 um; mobile phase: [water(FA)—ACN]; gradient:26%-56% B over 10 min) and lyophilized to give compound (R)-4-(6-(8-methyl-6-(3-methyl-3-phenylpyrrolidin-1-yl)-[1,2,4]triazolo[1,5-a]pyridine-2-carboxamido)-2-azaspiro[3.3]heptan-2-yl)picolinic acid (14.40 mg, 26.10 μmol, 73.83% yield, 100% purity) as a yellow solid.

LC-MS [ESI, M+1]: 552.2

¹H NMR (CHLOROFORM-d, 400 MHz): δ=8.23 (s, 0.8H), 7.71 (d, J=3.2 Hz, 0.9H), 7.61 (s, 1H), 7.27-7.39 (m, 4H), 7.21-7.25 (m, 1H), 7.13 (s, 1H), 6.97 (s, 1H), 6.32 (d, J=2.0 Hz, 1H), 4.45-4.70 (m, 1H), 3.96-4.41 (m, 4H), 3.32-3.59 (m, 4H), 2.79 (s, 2H), 2.58 (s, 3H), 2.45 (s, 2H), 2.31-2.37 (m, 1H), 2.23 (d, J=5.2 Hz, 1H), 1.43 (s, 3.0H)

| Compound | | |
|---|---|---|
| Number | Synthesis | LC/MS |
| 621 | Similar to | m/z 552.2 |
| | Example 112 | [M + H]⁺ |
| 692 | Similar to | m/z 580.4 |
| | Example 112 | [M + H]⁺ |
| 693 | Similar to | m/z 540.2 |
| | Example 112 | [M + H]⁺ |
| 694 | Similar to | m/z 540.3 |
| | Example 112 | [M + H]⁺ |

### Example 113

### 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylic acid (Compound 695)

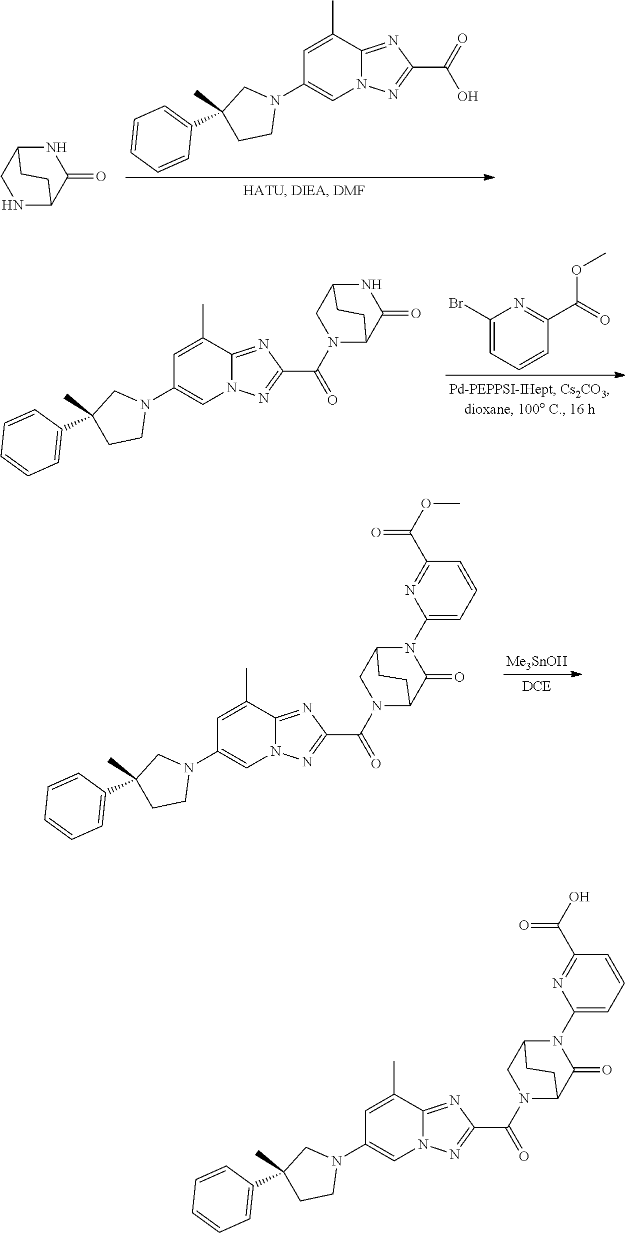

### Step 1: 5-[8-methyl-6-[(3R)-3-methyl-3-phenyl-pyrrolidin-1-yl]-[1,2,4]triazolo[1,5-a]pyridine-2-carbonyl]-2,5-diazabicyclo[2.2.2]octan-3-one

To a solution of 2,5-diazabicyclo[2.2.2]octan-3-one (41.25 mg, 327.00 μmol, 1.1 eq) and 8-methyl-6-[(3R)-3-methyl-3-phenyl-pyrrolidin-1-yl]-[1,2,4]triazolo[1,5-a]pyridine-2-carboxylic acid (100 mg, 297.28 μmol, 1 eq) in DMF (2 mL) was added DIEA (115.26 mg, 891.83 μmol, 155.34 μL, 3 eq) and HATU (169.55 mg, 445.92 μmol, 1.5 eq). The mixture was stirred at 25° C. for 0.5 h. LCMS showed starting material was consumed and the desired mass was detected. The reaction was quenched by water (0.1 mL). The crude product was purified by reversed phase—HPLC (0.1% FA condition) to give compound 5-[8-methyl-6-[(3R)-3-methyl-3-phenyl-pyrrolidin-1-yl]-[1,2,4]triazolo[1,5-a]pyridine-2-carbonyl]-2,5-diazabicyclo[2.2.2]octan-3-one (130 mg, 98% yield) as yellow solid.

LC-MS [ESI, M+1]: 445.3.

¹H NMR (400 MHz, CHLOROFORM-d) δ=7.69-7.60 (m, 1H), 7.39-7.28 (m, 4H), 7.24 (br s, 1H), 6.99 (s, 1H), 6.86-6.60 (m, 1H), 4.12-3.38 (m, 8H), 2.63 (s, 3H), 2.50-2.41 (m, 1H), 2.40-2.33 (m, 1H), 2.29-2.22 (m, 1H), 2.08-1.99 (m, 2H), 1.90-1.84 (m, 1H), 1.46 (s, 3H).

### Step 2: methyl 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylate

A mixture of 5-[8-methyl-6-[(3R)-3-methyl-3-phenyl-pyrrolidin-1-yl]-[1,2,4]triazolo[1,5-a]pyridine-2-carbonyl]-2,5-diazabicyclo[2.2.2]octan-3-one (120 mg, 269.95 μmol, 1 eq), methyl 6-bromopyridine-2-carboxylate (75.81 mg, 350.93 μmol, 1.3 eq), Cs₂CO₃ (263.86 mg, 809.85 μmol, 3 eq), 1,3-bis[2,6-bis(1-propylbutyl)phenyl]-4,5-dichloro-2H-imidazol-1-ium-2-ide;3-chloropyridine; dichloropalladium (26.26 mg, 26.99 μmol, 0.1 eq) in dioxane (2 mL) was degassed and purged with N₂ for 3 times, and then the mixture was stirred at 120° C. for 16 h under N₂ atmosphere. LCMS showed starting material was consumed and the desired mass was detected. The mixture was filtered and concentrated to give a residue. The crude product was purified by reversed phase-HPLC (0.1% FA condition) to give compound methyl 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylate (90 mg, 58% yield) as white solid.

LC-MS [ESI, M+1]: 580.3.

¹H NMR (400 MHz, DMSO-d₆) δ=8.43-8.26 (m, 1H), 8.02 (t, J=8.0 Hz, 1H), 7.94-7.81 (m, 2H), 7.45-7.19 (m, 6H), 5.75-5.55 (m, 1H), 5.36-5.12 (m, 1H), 4.17-3.96 (m, 1H), 3.92 (br s, 3H), 3.75 (q, J=11.6 Hz, 1H), 3.62-3.49 (m, 3H), 3.41 (br s, 1H), 2.57 (br s, 3H), 2.40-2.22 (m, 3H), 2.14-1.98 (m, 3H), 1.40 (s, 3H).

### Step 3: 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylic acid

To a solution of methyl 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylate (19 mg, 32.78 μmol, 1 eq) in DCE (1 mL) was added hydroxy(trimethyl)stannane (59.27 mg, 327.79 μmol, 10 eq). The mixture was stirred at 60° C. for 16 h. LCMS showed starting material was consumed and the desired mass was detected. The reaction was quenched by KF (0.02 mg). The reaction mixture was partitioned between EtOAc (5 mL×3) and water (10 mL), the combined organic phase was dried over anhydrous sodium sulfate, filtered and concentrated to give a residue. The crude product was purified by reversed phase-HPLC (column: CD04-Welch Ultimate C18 150*25 mm*7 um; mobile phase: [H₂O (0.225% FA)—ACN]; gradient:35%-65% B over 15.0 min) to give compound 6-[5-[8-methyl-6-[(3R)-3-methyl-3-phenyl-pyrrolidin-1-yl]-[1,2,4]triazolo[1,5-a]pyridine-2-carbonyl]-3-oxo-2,5-diazabicyclo[2.2.2]octan-2-yl]pyridine-2-carboxylic acid (5.76 mg, 31% yield) as white solid.

LC-MS [ESI, M+1]: 566.4.

¹H NMR (400 MHz, DMSO-d₆) δ=8.37-8.22 (m, 1H), 8.06-7.91 (m, 2H), 7.87-7.80 (m, 1H), 7.43-7.30 (m, 5H), 7.27-7.20 (m, 1H), 5.84-5.67 (m, 1H), 5.38-5.11 (m, 1H), 4.14-3.95 (m, 1H), 3.80-3.67 (m, 1H), 3.65-3.58 (m, 1H), 3.57-3.47 (m, 2H), 3.46-3.41 (m, 1H), 2.56 (s, 3H), 2.33-2.18 (m, 3H), 2.13-1.91 (m, 3H), 1.35 (d, J=6.0 Hz, 3H).

### Example 114

### 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinic acid (Compound 696)

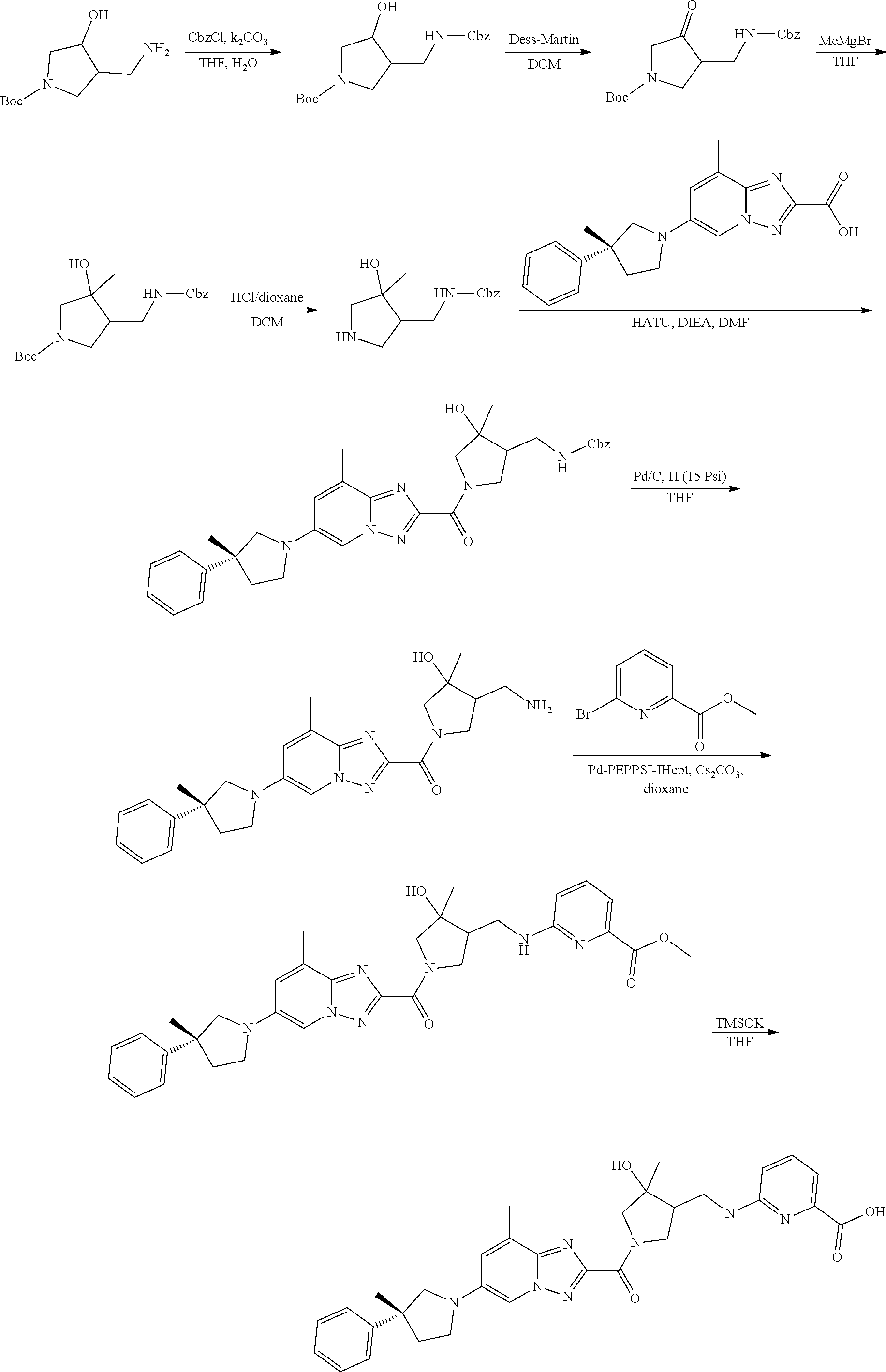

### Step 1: tert-butyl 3-(benzyloxycarbonylaminomethyl)-4-hydroxy-pyrrolidine-1-carboxylate

To a solution of tert-butyl 3-(aminomethyl)-4-hydroxy-pyrrolidine-1l-carboxylate (1.035 g, 4.79 mmol, 1 eq) in TIFF (5 mL) and H₂O (5 mL) was added CbzCl (816.38 mg, 4.79 mmol, 683.16 μL, 1 eq) and K₂CO₃ (661.39 mg, 4.79 mmol, 1 eq) at 0° C. The mixture was stirred at 25° C. for 4 h. TLC (SiO₂, DCM:EtOAc=10:1, Rf=0.5) indicated reactant was consumed and one new spot formed. The reaction mixture was quenched by H₂O (5 mL) and extracted with DCM (5 mL×3), the combined organic layers dried over anhydrous sodium sulfate, filtered and the filtrate was concentrated under reduced pressure to give a residue. The residue was purified by flash silica gel chromatography (SiO₂, DCM:EtOAc=10:1, Rf=0.5) to give tert-butyl 3-(benzyloxycarbonylaminomethyl)-4-hydroxy-pyrrolidine-1-carboxylate (1.4 g, 83.49% yield) as a yellow gum.

¹H NMR (400 MHz, CHLOROFORM-d) δ=7.42-7.32 (m, 5H), 5.19-5.10 (m, 3H), 4.22 (s, 1H), 3.67-3.35 (m, 5H), 3.15-3.06 (m, 2H), 1.46 (s, 9H).

### Step 2: tert-butyl 3-(benzyloxycarbonylaminomethyl)-4-oxo-pyrrolidine-1-carboxylate

To a solution of tert-butyl 3-(benzyloxycarbonylaminomethyl)-4-hydroxy-pyrrolidine-1-carboxylate (700 mg, 2.00 mmol, 1 eq) in DCM (10 mL) was added Dess-Martin (1.69 g, 4.00 mmol, 1.24 mL, 2 eq) at 0° C. The resulting mixture was stirred at 25° C. for 1 h. TLC (SiO₂, PE:EtOAc=1:1, Rf=0.5) indicated reactant was consumed and one new spot formed. The reaction mixture was quenched by H₂O (5 mL) and extracted with EtOAc (5 mL×3), the combined organic layers dried over anhydrous sodium sulfate, filtered and the filtrate was concentrated under reduced pressure to give a residue. The residue was purified by flash silica gel chromatography (SiO₂, PE:EtOAc=1:1, Rf=0.5) to give compound tert-butyl 3 (benzyloxycarbonylaminomethyl)-4-oxo-pyrrolidine-1-carboxylate (490 mg, 70.41% yield) a yellow gum.

¹H NMR (400 MHz, CHLOROFORM-d) δ=7.40-7.31 (m, 5H), 5.22 (s, 1H), 5.10 (s, 2H), 4.16-4.07 (m, 1H), 3.92 (d, J=3.6 Hz, 1H), 3.68 (d, J=19.6 Hz, 1H), 3.57-3.48 (m, 1H), 3.48-3.40 (m, 1H), 3.35 (dd, J=9.2, 11.2 Hz, 1H), 2.84 (d, J=6.0 Hz, 1H), 1.49 (s, 9H).

### Step 3: tert-butyl 4-(benzyloxycarbonylaminomethyl)-3-hydroxy-3-methyl-pyrrolidine-1-carboxylate

To a solution of MeMgBr (3 M, 1.41 mL, 3 eq) was added dropwise tert-butyl 3-(benzyloxycarbonylaminomethyl)-4-oxo-pyrrolidine-1-carboxylate (490 mg, 1.41 mmol, 1 eq) in THF (4 mL) at 0° C. over 0.1 h under N₂ atmosphere. The resulting mixture was stirred at 0° C. for 2 h under N₂ atmosphere. TLC (SiO₂, PE:EtOAc=1:1, Rf=0.4) indicated reactant was consumed completely and some new spots formed. The cold reaction mixture was poured into NH₄Cl (3 mL).The reaction mixture was extracted with EtOAc (4 mL×3), the combined organic layers dried over anhydrous sodium sulfate, filtered and the filtrate was concentrated under reduced pressure to give a residue. The residue was purified by flash silica gel chromatography (SiO₂, PE:EtOAc=1:1, Rf=0.4) to give tert-butyl 4-(benzyloxycarbonylaminomethyl)-3-hydroxy-3-methyl-pyrrolidine-1-carboxylate (230 mg, 44.87% yield) as a yellow gum.

¹H NMR (400 MHz, CHLOROFORM-d) δ=7.36 (s, 5H), 5.23-5.04 (m, 3H), 3.69-3.56 (m, 1H), 3.50-3.26 (m, 3H), 3.26-3.00 (m, 2H), 2.42-2.19 (m, 1H), 1.46 (s, 9H), 1.33 (s, 3H).

### Step 4: benzyl N-[(4-hydroxy-4-methyl-pyrrolidin-3-yl)methyl]carbamate

To a solution of tert-butyl 4-(benzyloxycarbonylaminomethyl)-3-hydroxy-3-methyl-pyrrolidine-1-carboxylate (160 mg, 439.04 μmol, 1 eq) in DCM (1.5 mL) was added HCl/dioxane (2 M, 3 mL, 13.67 eq). The mixture was stirred at 25° C. for 0.5 h. LCMS showed starting material was consumed and the desired mass was detected. The mixture was concentrated under pressure to give compound benzyl N-[(4-hydroxy-4-methyl-pyrrolidin-3-yl)methyl]carbamate (132 mg, crude, HCl) as a red gum.

LC-MS [ELSD, M+1]: 265.2.

### Step 5: benzyl N-[[4-hydroxy-4-methyl-1-[8-methyl-6-[(3R)-3-methyl-3-phenyl-pyrrolidin-1-yl]-[1,2,4]triazolo[1,5-a]pyridine-2-carbonyl]pyrrolidin-3-yl]methyl]carbamate

To a solution of 8-methyl-6-[(3R)-3-methyl-3-phenyl-pyrrolidin-1-yl]-[1,2,4]triazolo[1,5-a]pyridine-2-carboxylic acid (134.21 mg, 398.96 μmol, 1 eq) in DMF (1.5 mL) was added HATU (227.55 mg, 598.44 μmol, 1.5 eq). The mixture was stirred at this temperature for 0.5 h, After addition of benzyl N-[(4-hydroxy-4-methyl-pyrrolidin-3-yl)methyl]carbamate (132 mg, 438.86 μmol, 1.1 eq, HCl) and DIEA (257.81 mg, 1.99 mmol, 347.45 μL, 5 eq) at 25° C. The resulting mixture was stirred at 25° C. for 1 h. LCMS showed starting material was consumed and one main peak with desired mass was detected. The mixture was quenched with H₂O (2 mL) and extracted with EtOAc (2 mL×3), the organic phase was dried, filtered, and concentrated under pressure to give the product. The residue was purified by flash silica gel chromatography (SiO₂, EtOAc:NH₃·MeOH (7 M)=40:1, Rf=0.3) to give compound benzyl N-[[4-hydroxy-4-methyl-1-[8-methyl-6-[(3R)-3-methyl-3-phenyl-pyrrolidin-1-yl]-[1,2,4]triazolo[1,5-a]pyridine-2-carbonyl]pyrrolidin-3-yl]methyl]carbamate (170 mg, 73.13% yield) as a yellow solid.

LC-MS [ESI, M+1]: 583.5.

### Step 6: [4-(aminomethyl)-3-hydroxy-3-methyl-pyrrolidin-1-yl]-[8-methyl-6-[(3R)-3-methyl-3-phenyl-pyrrolidin-1-yl]-[1,2,4]triazolo[1,5-a]pyridin-2-yl]methanone

To a mixture of benzyl N-[[4-hydroxy-4-methyl-1-[8-methyl-6-[(3R)-3-methyl-3-phenyl-pyrrolidin-1-yl]-[1,2,4]triazolo[1,5-a]pyridine-2-carbonyl]pyrrolidin-3-yl]methyl]carbamate (140 mg, 240.26 μmol, 1 eq) in THF (3 mL) was added Pd/C (25.57 mg, 24.03 μmol, 10% purity, 0.1 eq) then the mixture was stirred at 25° C. for 2 h under H₂ (15 Psi). LCMS showed starting material was consumed and one main peak with desired mass was detected. The mixture was filtered, and concentrated under pressure to give the product. The residue was purified by prep-HPLC (column: Phenomenex Luna C18 20 g; mobile phase: [H₂O (FA)—ACN]; B %: 30%-40%, 8 min) and lyophilized to give compound [4-(aminomethyl)-3-hydroxy-3-methyl-pyrrolidin-1-yl]-[8-methyl-6-[(3R)-3-methyl-3-phenyl-pyrrolidin-1-yl]-[1,2,4]triazolo[1,5-a]pyridin-2-yl]methanone (45 mg, 41.75% yield) as a white solid.

LC-MS [ESI, M+1]: 449.2.

¹H NMR (400 MHz, DMSO-d₆) δ=8.08-7.77 (m, 4H), 7.43-7.30 (m, 5H), 7.27-7.20 (m, 1H), 4.11 (dd, J=7.2, 11.6 Hz, 0.35H), 3.87 (dd, J=7.6, 12.8 Hz, 0.65H), 3.83-3.73 (m, 1H), 3.66-3.57 (m, 2H), 3.56-3.49 (m, 4H), 3.04-2.93 (m, 1H), 2.82-2.66 (m, 1H), 2.54 (d, J=4.8 Hz, 3H), 2.39-2.32 (m, 1H), 2.26 (t, J=6.8 Hz, 2H), 1.35 (s, 3H), 1.27-1.09 (m, 3H).

### Step 7: methyl 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinate

A mixture of [4-(aminomethyl)-3-hydroxy-3-methyl-pyrrolidin-1-yl]-[8-methyl-6-[(3R)-3-methyl-3-phenyl-pyrrolidin-1-yl]-[1,2,4]triazolo[1,5-a]pyridin-2-yl]methanone (35 mg, 78.03 μmol, 1 eq), methyl 6-bromopyridine-2-carboxylate (16.86 mg, 78.03 μmol, 1 eq), Cs₂CO₃ (50.85 mg, 156.06 μmol, 2 eq), [1,3-bis[2,6-bis(1-ethylpropyl)phenyl]-4,5-dichloro-imidazol-2-ylidene]-dichloro-(2-methylpyridin-1-ium-1-yl)palladium (6.56 mg, 7.80 μmol, 0.1 eq) in dioxane (1 mL) was degassed and purged with N₂ for 3 times, and then the mixture was stirred at 100° C. for 2 h under N₂ atmosphere. LCMS showed starting material was consumed and one main peak with desired mass was detected. The reaction solution was concentrated under the reduced pressure to give the residue. The residue was purified by prep-HPLC (column: Phenomenex Luna C18 12 g;mobile phase: [H₂O (FA)—ACN]; B %: 42%-50%, 8 min.) and lyophilized to give compound methyl 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinate (30 mg, 65.87% yield) as a white solid.

LC-MS [ESI, M+1]: 584.4.

### Step 8: 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinic acid

To a solution of methyl 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinate (20 mg, 34.27 μmol, 1 eq) in THF (1 mL) was added TMSOK (8.79 mg, 68.53 μmol, 2 eq). The mixture was stirred at 25° C. for 0.5 h. LCMS showed starting material was consumed and one main peak with desired mass was detected. Acidify the clear solution with FA. The reaction solution was concentrated under the reduced pressure to give the residue. The residue was purified by prep-HPLC (column: CD01-Phenomenex luna C18 150*25*10 um; mobile phase: [H₂O (0.225% FA)—ACN]; gradient:20%-50% B over 10.0 min.) and lyophilized to give compound 6-(((4-hydroxy-4-methyl-1-(8-methyl-6-((R)-3-methyl-3-phenylpyrrolidin-1-yl)-[1,2,4]triazolo[1,5-a]pyridine-2-carbonyl)pyrrolidin-3-yl)methyl)amino)picolinic acid (10.95 mg, 56.10% yield) as a white solid.

LC-MS [ESI, M+1]: 570.4.

¹H NMR (400 MHz, DMSO-d₆) δ=7.95-7.83 (m, 1H), 7.60-7.45 (m, 1H), 7.44-7.32 (m, 4H), 7.32-7.27 (m, 1H), 7.26-7.12 (m, 2H), 7.04-6.78 (m, 1H), 6.77-6.59 (m, 1H), 5.12 (s, 1H), 4.08 (dd, J=7.2, 11.2 Hz, 0.5H), 3.85-3.81 (m, 0.5H), 3.80-3.66 (m, 2H), 3.64-3.56 (m, 2H), 3.55-3.47 (m, 5H), 2.54 (s, 3H), 2.43-2.30 (m, 1H), 2.30-2.19 (m, 2H), 1.41-1.21 (m, 6H).

### Example 115

### 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylic acid (Compound 697)

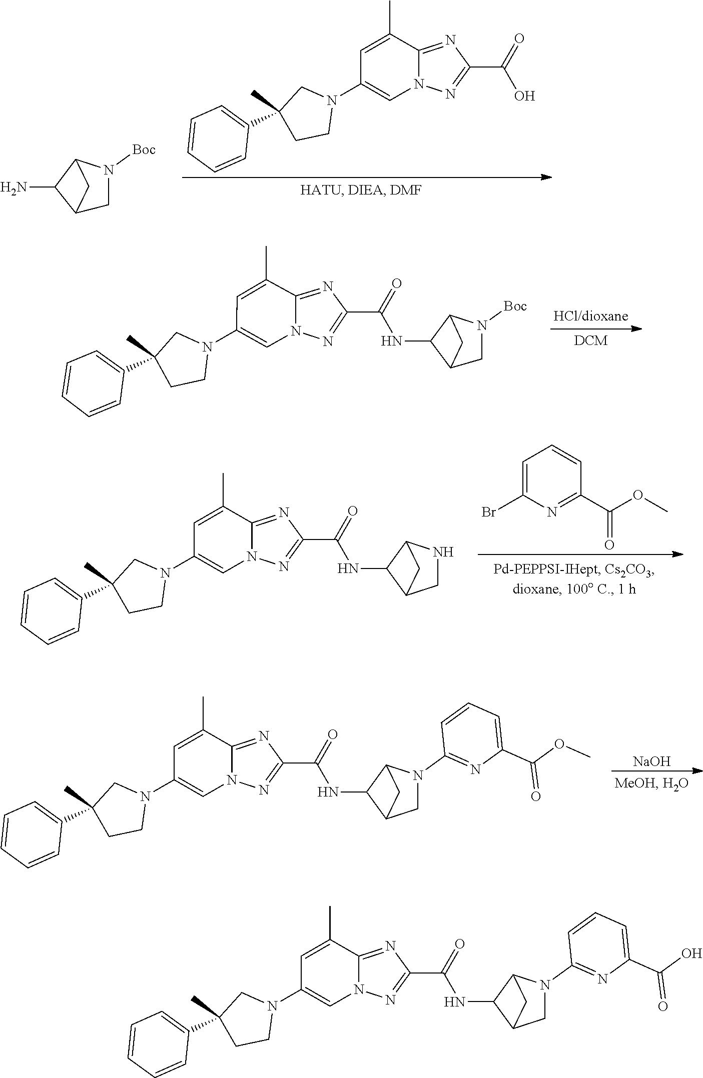

### Step 1: tert-butyl 5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexane-2-carboxylate

To a solution of 8-methyl-6-[(3R)-3-methyl-3-phenyl-pyrrolidin-1-yl]-[1,2,4]triazolo[1,5-a]pyridine-2-carboxylic acid (100 mg, 297.28 μmol, 1 eq) and tert-butyl 5-amino-2-azabicyclo[2.1.1]hexane-2-carboxylate (58.94 mg, 297.28 μmol, 1 eq) in DMF (1.5 mL) was added HATU (169.55 mg, 445.92 μmol, 1.5 eq) and DIEA (115.26 mg, 891.83 μmol, 155.34 μL, 3 eq). The mixture was stirred at 25° C. for 1 h. TLC (PE:EtOAc=0:1) showed reactant was consumed completely and the three new spots formed. The reaction was added H₂O (2 mL) and then extracted with EtOAc (10 mL×3).The combined organic phase was washed with brine (5 mL×5), dried over anhydrous Na₂SO₄, filtered and concentrated in vacuum. The residue was purified by prep-HPLC (column: CD01-Phenomenex luna C18 150*25*10 um; mobile phase: [H₂O (0.1% FA)—ACN]; gradient:50%-62% B over 10.0 min) to give compound tert-butyl 5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexane-2-carboxylate (130 mg, 85% yield) as a yellow oil.

¹H NMR (400 MHz, CHLOROFORM-d) δ=7.64 (d, J=1.2 Hz, 1H), 7.45 (d, J=5.6 Hz, 1H), 7.40-7.34 (m, 2H), 7.34-7.29 (m, 2H), 7.27-7.24 (m, 1H), 7.00 (s, 1H), 4.43 (s, 1H), 4.29 (s, 1H), 3.60 (d, J=8.8 Hz, 1H), 3.57-3.45 (m, 3H), 3.45-3.37 (m, 2H), 3.04 (s, 1H), 2.61 (s, 3H), 2.43-2.36 (m, 1H), 2.28 (dd, J=7.2, 4.0 Hz, 1H), 1.71 (d, J=8.0 Hz, 1H), 1.48 (d, J=12.4 Hz, 12H), 1.32 (d, J=8.4 Hz, 1H).

### Step 2: N-(2-azabicyclo[2.1.1]hexan-5-yl)-8-methyl-6-[(3R)-3-methyl-3-phenyl-pyrrolidin-1-yl]-[1,2,4]triazolo[1,5-a]pyridine-2-carboxamide

To a solution of tert-butyl 5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexane-2-carboxylate (50 mg, 96.78 μmol, 1 eq) in DCM (0.5 mL) was added HCl/dioxane (2 M, 0.2 mL, 4.13 eq). The mixture was stirred at 25° C. for 0.5 h. LCMS showed reactant was consumed completely and the desired compound was detected. The reaction mixture was concentrated in vacuum. The mixture was added DCM/MeOH (10:1) (10 mL) and NaHCO₃ solid until pH around 7. The mixture was filtered and the filtrated was concentrated in vacuum to give compound N-(2-azabicyclo[2.1.1]hexan-5-yl)-8-methyl-6-[(3R)-3-methyl-3-phenyl-pyrrolidin-1-yl]-[1,2,4]triazolo[1,5-a]pyridine-2-carboxamide (50 mg, crude) as a yellow oil.

LC-MS [ESI, M+1]: 417.2.

### Step 3: methyl 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylate

To a solution of N-(2-azabicyclo[2.1.1]hexan-5-yl)-8-methyl-6-[(3R)-3-methyl-3-phenyl-pyrrolidin-1-yl]-[1,2,4]triazolo[1,5-a]pyridine-2-carboxamide (40 mg, 96.03 μmol, 1 eq) and methyl 6-bromopyridine-2-carboxylate (20.75 mg, 96.03 μmol, 1 eq) in dioxane (1 mL) was added [1,3-bis[2,6-bis(1-ethylpropyl)phenyl]-4,5-dichloroimidazol-2-ylidene]-dichloro-(2-methylpyridin-1-ium-1-yl)palladium (8.07 mg, 9.60 μmol, 0.1 eq) and Cs₂CO₃ (156.45 mg, 480.17 μmol, 5 eq). The mixture was stirred at 100° C. for 2 h under N₂. LCMS showed reactant was consumed completely and the desired compound was detected. The mixture was filtered and the filtrated was concentrated in vacuum. The residue was purified by prep-HPLC (column: CD01-Phenomenex luna C18 150*25*10 um; mobile phase: [H₂O (0.225% FA)—ACN]; gradient:60%-70% B over 10.0 min) to give compound methyl 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylate (35 mg, 65% yield) as a yellow solid.

LC-MS [ESI, M+1]: 552.3.

### Step 4: 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylic acid

To a solution of methyl 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylate (30 mg, 54.38 μmol, 1 eq) in MeOH (0.5 mL) was added NaOH (4.35 mg, 108.77 μmol, 2 eq) and H₂O (0.1 mL). The mixture was stirred at 25° C. for 0.5 h. LCMS showed reactant was consumed completely and the desired compound was detected. The reaction mixture was added FA until pH around 4. The residue was purified by prep-HPLC (column: CD01-Phenomenex luna C18 150*25*10 um; mobile phase: [H₂O (0.225% FA)—ACN]; gradient:28%-58% B over 10.0 min) to give compound 6-[5-[[8-methyl-6-[(3R)-3-methyl-3-phenyl-pyrrolidin-1-yl]-[1,2,4]triazolo[1,5-a]pyridine-2-carbonyl]amino]-2-azabicyclo[2.1.1]hexan-2-yl]pyridine-2-carboxylic acid (25.44 mg, 86% yield) as a yellow solid.

LC-MS [ESI, M+1]: 538.3.

¹H NMR (400 MHz, CHLOROFORM-d) δ=7.71-7.63 (m, 1H), 7.58 (d, J=1.6 Hz, 1H), 7.53 (d, J=7.2 Hz, 1H), 7.41 (d, J=7.2 Hz, 1H), 7.39-7.33 (m, 2H), 7.32-7.28 (m, 2H), 7.27-7.22 (m, 1H), 6.96 (s, 1H), 6.85 (d, J=8.4 Hz, 1H), 4.84 (d, J=6.4 Hz, 1H), 4.45 (td, J=7.2, 2.4 Hz, 1H), 3.65-3.56 (m, 2H), 3.55-3.43 (m, 3H), 3.39 (dt, J=8.8, 3.2 Hz, 1H), 3.21 (td, J=6.4, 3.2 Hz, 1H), 2.54 (s, 3H), 2.37 (td, J=12.4, 8.4 Hz, 1H), 2.29-2.21 (m, 1H), 1.84 (d, J=8.4 Hz, 1H), 1.45 (s, 3H), 1.40 (d, J=8.4 Hz, 1H).

| Compound | | |
|---|---|---|
| Number | Synthesis | LC/MS |
| 698 | Similar to | m/z 552.3 |
| | Example 115 | [M + H]⁺ |

### Example 116

### 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylic acid (Compound 699)

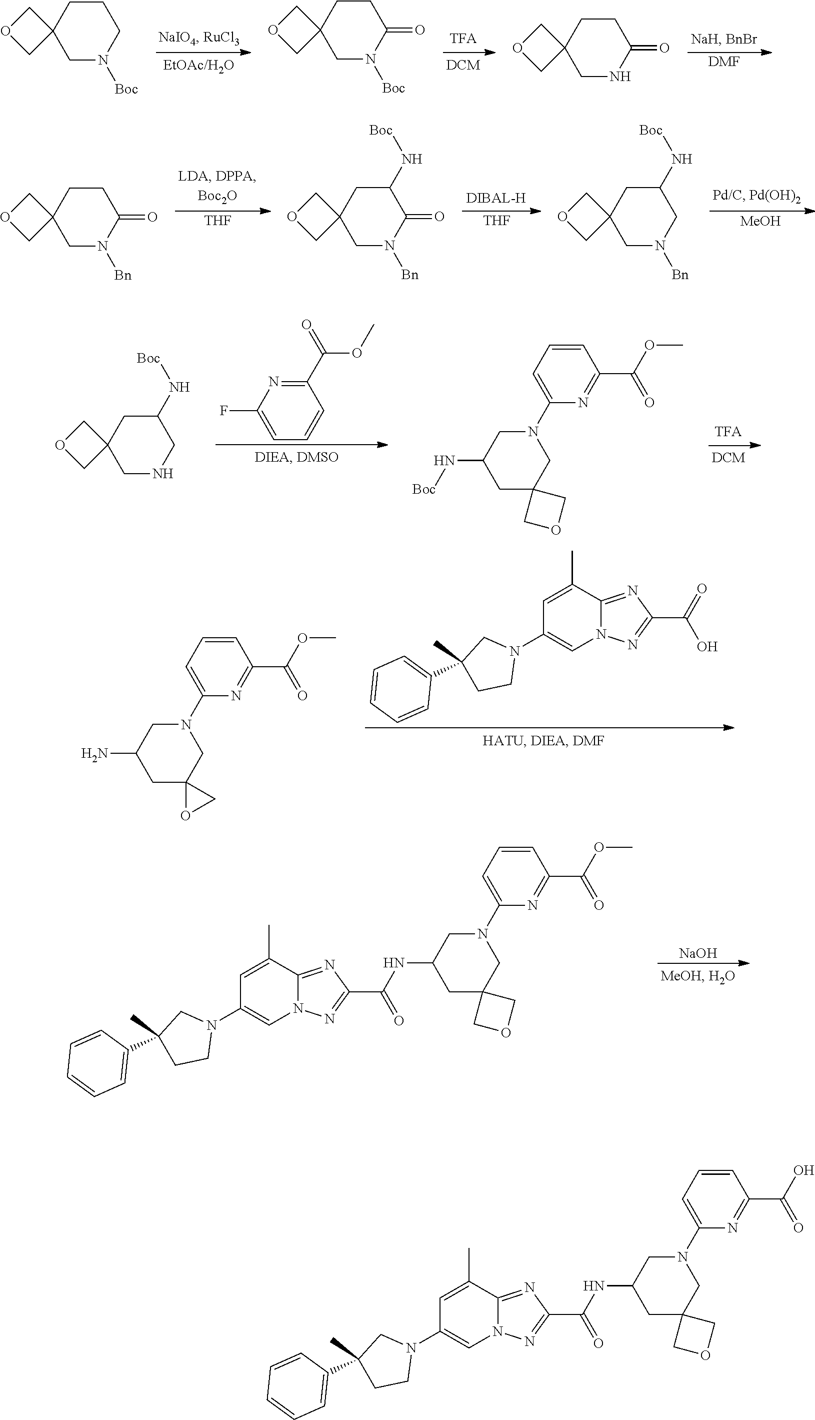

### Step 1: Step 1: tert-butyl 7-oxo-2-oxa-8-azaspiro[3.5]nonane-8-carboxylate

To a solution of NaIO₄ (10.35 g, 48.39 mmol, 2.68 mL, 2.2 eq) in H₂O (50 mL) was added RuCl₃ (456.29 mg, 2.20 mmol, 146.72 μL, 0.1 eq) and then tertbutyl 2-oxa-8-azaspiro[3.5]nonane-8-carboxylate (5 g, 22.00 mmol, 1 eq) in EtOAc (50 mL) was added dropwise at 0° C. under N₂. The reaction mixture was stirred at 25° C. for 2 h. TLC (PE:EtOAc=1:1) showed reactant was not consumed completely and the new spots was detected. The reaction was quenched by Na₂SO₃ (60 mL) and then extracted with EtOAc (50 mL×5). The combined organic phase was dried over anhydrous Na₂SO₄, filtered and concentrated in vacuum. The residue was purified by column chromatography (SiO₂, PE:EtOAc=1:1, Rf=0.23) to give compound tert-butyl 7-oxo-2-oxa-8-azaspiro[3.5]nonane-8-carboxylate (1.5 g, 28% yield) as a yellow solid

¹H NMR (400 MHz, CHLOROFORM-d) δ=4.62-4.40 (m, 4H), 3.92 (s, 2H), 2.51 (t, J=7.2 Hz, 2H), 2.11 (t, J=7.2 Hz, 2H), 1.54 (s, 9H).

### Step 2: 2-oxa-8-azaspiro[3.5]nonan-7-one

To a solution of tert-butyl 7-oxo-2-oxa-8-azaspiro[3.5]nonane-8-carboxylate (1.5 g, 6.22 mmol, 1 eq) in DCM (10 mL) was added TFA (3.14 g, 27.54 mmol, 2.05 mL, 4.43 eq). The mixture was stirred at 25° C. for 0.5 h. LCMS showed reactant was consumed completely. The reaction mixture was concentrated in vacuum to give compound 2-oxa-8-azaspiro[3.5]nonan-7-one (1.5 g, crude, TFA) as a yellow oil.

LC-MS [ESI, M+1]: 142.2.

### Step 3: 8-benzyl-2-oxa-8-azaspiro[3.5]nonan-7-one

To a solution of 2-oxa-8-azaspiro[3.5]nonan-7-one (1.5 g, 5.88 mmol, 1 eq, TFA) in THF (15 mL) was added NaH (587.74 mg, 14.69 mmol, 60% purity, 2.5 eq). The mixture was stirred at 0° C. for 0.5 h. Then the solution was added dropwise BnBr (1.21 g, 7.05 mmol, 837.78 μL, 1.2 eq) at 0° C. The mixture was stirred at 25° C. for 1 h under N₂. LCMS showed reactant was consumed completely and the desired mass was detected. The reaction was quenched by NH₄Cl (20 mL) slowly and then extracted with ethyl acetate (15 mL×3).The combined organic phase was washed with brine (10 mL×3), dried with anhydrous Na₂SO₄, filtered and concentrated in vacuum The residue was purified by prep-HPLC (column: CD01-Phenomenex luna C18 150*25*10 um; mobile phase: [H₂O (0.1% FA)—ACN]; gradient:30%-40% B over 15.0 min) to give compound 8-benzyl-2-oxa-8-azaspiro[3.5]nonan-7-one (620 mg, 46% yield) was obtained as a yellow solid.

LC-MS [ESI, M+1]: 232.2.

¹H NMR (400 MHz, CHLOROFORM-d) δ=7.27 (s, 5H), 4.61 (s, 2H), 4.49 (d, J=6.4 Hz, 2H), 4.31 (d, J=6.4 Hz, 2H), 3.45 (s, 2H), 2.50 (t, J=6.8 Hz, 2H), 2.09 (t, J=6.8 Hz, 2H).

### Step 4: tert-butyl N-(8-benzyl-7-oxo-2-oxa-8-azaspiro[3.5]nonan-6-yl)carbamate

To a solution of 8-benzyl-2-oxa-8-azaspiro[3.5]nonan-7-one (600 mg, 2.59 mmol, 1 eq) in THF (8 mL) was added LDA (2 M, 3.24 mL, 2.5 eq). The mixture was stirred at −78° C. for 0.5 h under N₂. Then the solution was added DPPA (1.43 g, 5.19 mmol, 1.12 mL, 2 eq). The mixture was stirred at −78° C. for 0.5 h under N₂. After the mixture was added Boc₂O (849.25 mg, 3.89 mmol, 893.94 μL, 1.5 eq) in THF (2 mL). The mixture was stirred at −78° C. for 0.5 h. LCMS showed reactant was consumed completely and the desired mass was detected. The reaction was quenched by NH₄Cl (8 mL) and then extracted with EtOAc (10 mL×5). The combined organic phase was dried over anhydrous Na₂SO₄, filtered and concentrated in vacuum. The residue was purified by prep-HPLC (column: CD01-Phenomenex luna C18 150*25*10 um; mobile phase: [H₂O (0.1% FA)—ACN]; gradient:25%-35% B over 10.0 min) to give compound tert-butyl N-(8-benzyl-7-oxo-2-oxa-8-azaspiro[3.5]nonan-6-yl)carbamate (375 mg, 42% yield) as a yellow solid.

LC-MS [ESI, M-55]: 291.1.

¹H NMR (400 MHz, CHLOROFORM-d) δ=7.40-7.31 (m, 3H), 7.26-7.22 (m, 2H), 5.55 (s, 1H), 4.70 (d, J=6.0 Hz, 1H), 4.67-4.53 (m, 2H), 4.44 (d, J=6.4 Hz, 1H), 4.34 (d, J=6.0 Hz, 1H), 4.13 (d, J=6.4 Hz, 1H), 4.07-3.92 (m, 1H), 3.63 (dd, J=12.4, 1.2 Hz, 1H), 3.41 (d, J=12.6 Hz, 1H), 2.90 (br dd, J=12.8, 2.6 Hz, 1H), 1.80 (t, J=12.6 Hz, 1H), 1.47 (s, 9H)

### Step 5:tert-butyl N-(8-benzyl-2-oxa-8-azaspiro[3.5]nonan-6-yl)carbamate

To a solution of DIBAL-H (1 M, 4.27 mL, 4 eq) in THF (4 mL) was added tert-butyl N-(8-benzyl-7-oxo-2-oxa-8-azaspiro[3.5]nonan-6-yl)carbamate (370 mg, 1.07 mmol, 1 eq). The mixture was stirred at −78° C. for 5 min under N₂. LCMS showed reactant was consumed completely and the desired mass was detected. After the reaction mixture was cooled to 0° C., the reaction mixture was quenched by addition of 1 mL of H₂O, followed by 1 mL of 15% aqueous NaOH and 3 mL of H₂O. After being stirred at room temperature for 0.5 h, the solid was removed by filtration. The filtrate was concentrated to dryness to give crude product. The residue was purified by prep-HPLC (column: CD01-Phenomenex luna C18 150*25*10 um; mobile phase: [H₂O (0.1% FA)—ACN]; gradient:20%-25% B over 15.0 min) to give compound tert-butyl N-(8-benzyl-2-oxa-8-azaspiro[3.5]nonan-6-yl)carbamate (75 mg, 21% yield) as a yellow oil.

LC-MS [ESI, M-55]: 333.2.

¹H NMR (400 MHz, CHLOROFORM-d) δ=7.39-7.28 (m, 5H), 4.79-4.54 (m, 1H), 4.46 (d, J=5.6 Hz, 2H), 4.39-4.22 (m, 2H), 3.70 (s, 1H), 3.61-3.47 (m, 2H), 2.87-2.39 (m, 3H), 2.37-2.12 (m, 1H), 2.00-1.77 (m, 2H), 1.51-1.41 (m, 9H).

### Step 6:tert-butyl N-(2-oxa-8-azaspiro[3.5]nonan-6-yl)carbamate

To a solution of tert-butyl N-(8-benzyl-2-oxa-8-azaspiro[3.5]nonan-6-yl)carbamate (50 mg, 150.40 μmol, 1 eq) in MeOH (3 mL) was added Pd/C (16.01 mg, 15.04 μmol, 10% purity, 0.1 eq) and Pd(OH)₂ (10.56 mg, 15.04 μmol, 20% purity, 0.1 eq). The mixture was stirred at 50° C. for 2 h under H₂ (50 Psi). LCMS showed reactant was consumed completely and the desired mass was detected. The reaction mixture was filtered and the filtrated was concentrated in vacuum to give compound tert-butyl N-(2-oxa-8-azaspiro[3.5]nonan-6-yl)carbamate (40 mg, crude) as a yellow oil.

LC-MS [ESI, M+1]: 243.2.

### Step 7: methyl 6-[6-(tert-butoxycarbonylamino)-2-oxa-8-azaspiro[3.5]nonan-8-yl]pyridine-2-carboxylate

To a solution of tert-butyl N-(2-oxa-8-azaspiro[3.5]nonan-6-yl)carbamate (40 mg, 165.08 μmol, 1 eq) and methyl 6-fluoropyridine-2-carboxylate (25.61 mg, 165.08 μmol, 1 eq) in DMSO (1.5 mL) was added DIEA (64.00 mg, 495.23 μmol, 86.26 μL, 3 eq). The mixture was stirred at 120° C. for 2 h. LCMS showed reactant was consumed completely and the desired compound was detected. The reaction mixture was filtered. The residue was purified by prep-HPLC (column: CD01-Phenomenex luna C18 150*25 mm*10 um; mobile phase: [H₂O (0.1% FA)—ACN]; gradient:35%-100% B over 10.0 min) to give compound methyl 6-[6-(tert-butoxycarbonylamino)-2-oxa-8-azaspiro[3.5]nonan-8-yl]pyridine-2-carboxylate (33 mg, 53% yield) as a yellow oil.

LC-MS [ESI, M-55]: 320.2.

### Step 8: methyl 6-(6-amino-2-oxa-8-azaspiro[3.5]nonan-8-yl)pyridine-2-carboxylate

To a solution of methyl 6-[6-(tert-butoxycarbonylamino)-2-oxa-8-azaspiro[3.5]nonan-8-yl]pyridine-2-carboxylate (22 mg, 58.29 μmol, 1 eq) in DCM (2 mL) was added TFA (153.50 mg, 1.35 mmol, 0.1 mL, 23.10 eq). The mixture was stirred at 25° C. for 0.5 h. LCMS showed reactant was consumed completely and the desired compound was detected. The reaction mixture was concentrated in vacuum to give compound methyl 6-(6-amino-2-oxa-8-azaspiro[3.5]nonan-8-yl)pyridine-2-carboxylate (21 mg, crude, TFA) as a yellow oil.

LC-MS [ESI, M-56]: 278.2.

### Step 9: methyl 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (15 mg, 44.59 μmol, 1 eq) and methyl 6-(6-amino-2-oxa-8-azaspiro[3.5]nonan-8-yl)pyridine-2-carboxylate (20.94 mg, 53.51 μmol, 1.2 eq, TFA) in DMF (1.5 mL) was added HATU (25.43 mg, 66.89 μmol, 1.5 eq) and DIEA (28.82 mg, 222.96 μmol, 38.83 μL, 5 eq). The mixture was stirred at 25° C. for 0.5 h. LCMS showed reactant was consumed completely and the desired compound was detected. The reaction mixture was filtered The residue was purified by prep-HPLC (column: CD01-Phenomenex luna C18 150*25 mm*10 um; mobile phase: [H₂O (0.1% FA)—ACN]; gradient:35%-50% B over 10.0 min) to give compound methyl 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylate (22 mg, 83% yield) as a yellow solid.

LC-MS [ESI, M+1]: 596.5.

### Step 10: 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylic acid

To a solution of methyl 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylate (20 mg, 33.57 μmol, 1 eq) in MeOH (1 mL) was added NaOH (2.69 mg, 67.15 μmol, 2 eq) and H₂O (0.1 mL). The mixture was stirred at 25° C. for 0.5 h. LCMS showed reactant was consumed completely and the desired compound was detected. The reaction mixture was added FA until pH around 4. The residue was purified by prep-HPLC (column: CD25-WePure Biotech XPT PHS C18 150*25*7 um; mobile phase: [H₂O (0.225% FA)—ACN]; gradient:39%-69% B over 10.0 min) to give compound 6-[6-[[8-methyl-6-[(3R)-3-methyl-3-phenyl-pyrrolidin-1-yl]-[1,2,4]triazolo[1,5-a]pyridine-2-carbonyl]amino]-2-oxa-8-azaspiro[3.5]nonan-8-yl]pyridine-2-carboxylic acid (2.78 mg, 14% yield) was obtained as a white solid.

LC-MS [ESI, M+1]: 582.5.

¹H NMR (400 MHz, DMSO-d₆) δ=8.49 (d, J=8.0 Hz, 1H), 7.91 (s, 1H), 7.67 (t, J=7.6 Hz, 1H), 7.48-7.32 (m, 5H), 7.29 (d, J=6.8 Hz, 1H), 7.27-7.20 (m, 1H), 7.16 (d, J=8.0 Hz, 1H), 4.73 (d, J=12.4 Hz, 1H), 4.46-4.29 (m, 4H), 4.29-4.20 (m, 1H), 3.94-3.85 (m, 1H), 3.64-3.59 (m, 1H), 3.57-3.47 (m, 3H), 2.99 (d, J=13.2 Hz, 1H), 2.90 (t, J=11.2 Hz, 1H), 2.56 (s, 3H), 2.35-2.30 (m, 1H), 2.30-2.24 (m, 2H), 2.03-1.90 (m, 1H), 1.36 (s, 3H).

| Compound | | |
|---|---|---|
| Number | Synthesis | LC/MS |
| 700 | Similar to | m/z 600.5 |
| | Example 116 | [M + H]⁺ |
| 701 | Similar to | m/z 640.4 |
| | Example 116 | [M + H]⁺ |
| 702 | Similar to | m/z 582.4 |
| | Example 116 | [M + H]⁺ |

### Example 117

### Mis6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (Compound 703)

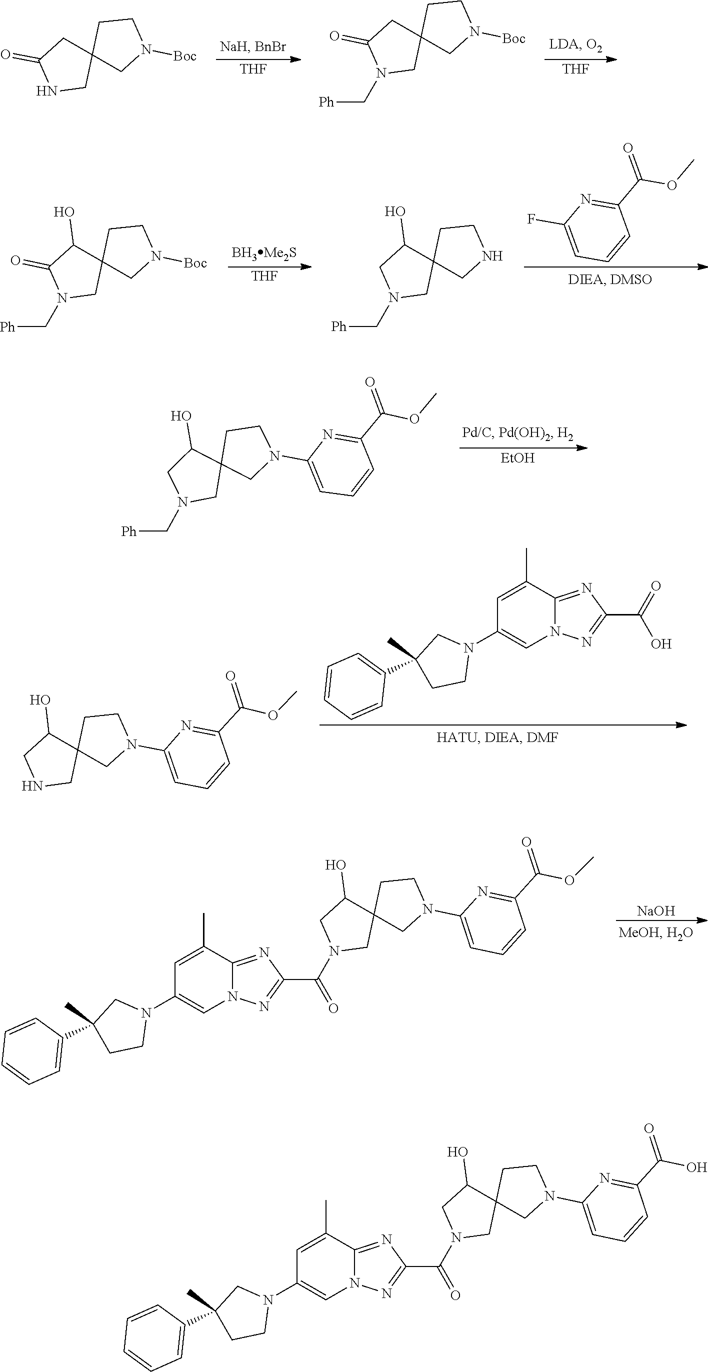

### Step 1: tert-butyl 2-benzyl-3-oxo-2,7-diazaspiro[4.4]nonane-7-carboxylate

To a solution of tert-butyl 8-oxo-2,7-diazaspiro[4.4]nonane-2-carboxylate (2 g, 8.32 mmol, 1 eq) in DMF (20 mL) was added dropwise NaH (499.33 mg, 12.48 mmol, 6000 purity, 1.5 eq) at 0° C. After addition, the mixture was stirred at this temperature for 30 min, and then BnBr (1.71 g, 9.99 mmol, 1.19 mL, 1.2 eq) was added dropwise. The resulting mixture was stirred at 25° C. for 30 min. LCMS showed starting material was consumed. Several new peaks were shown on LCMS and 93% of desired compound was detected. The reaction mixture was quenched by addition NH₄Cl (5 mL) at 0° C., and then the reaction mixture was filtered and then diluted with H₂O (10 mL) and extracted with EtOAc (30 mL×3). The combined organic layers were washed with brine 15 mL (5 mL×3), dried over Na₂SO₄, filtered and concentrated under reduced pressure to give a residue. The residue was purified by flash silica gel chromatography (SiO₂, PE:EtOAc=1:2, Rf=0.3) to give compound tert-butyl 2-benzyl-3-oxo-2,7-diazaspiro[4.4]nonane-7-carboxylate (2.42 g, 88.00% yield) as yellow oil.

LC-MS [ESI, M-55]: 275.1.

¹H NMR (400 MHz, CHLOROFORM-d) δ=7.27 (s, 5H), 4.62-4.32 (m, 2H), 3.49-3.06 (m, 6H), 2.59-2.34 (m, 2H), 1.97-1.77 (m, 2H), 1.43 (br d, J=4.4 Hz, 9H)

### Step 2: tert-butyl 2-benzyl-4-hydroxy-3-oxo-2,7-diazaspiro[4.4]nonane-7-carboxylate

To a solution of tert-butyl 2-benzyl-3-oxo-2,7-diazaspiro[4.4]nonane-7-carboxylate (2.42 g, 7.32 mmol, 1 eq) in THF (50 mL) was added dropwise LDA (2 M, 7.32 mL, 2 eq) at −78° C. under N₂. After addition, the mixture was stirred at this temperature for 30 min, and then oxygen (234.36 mg, 7.32 mmol, 1 eq) was introduced in. The resulting mixture was stirred at −78° C. for 12 h. LCMS showed starting material was consumed. Several new peaks were shown on LCMS and the 56% of desired compound was detected. The reaction mixture was quenched by addition NH₄Cl (10 mL) mL at 0° C., and then the reaction mixture was filtered and then diluted with H₂O (10 mL) and extracted with EtOAc (20 mL×3). The combined organic layers were washed with brine (5 mL×3), dried over Na₂SO₄, filtered and concentrated under reduced pressure to give a residue. The crude product was purified by reversed-phase HPLC (0.1% FA condition) to give compound tert-butyl 2-benzyl-4-hydroxy-3-oxo-2,7-diazaspiro[4.4]nonane-7-carboxylate (820 mg, 32.32% yield) as white solid.

LC-MS [ESI, M-55]: 291.0.

¹H NMR (400 MHz, CHLOROFORM-d) δ=7.43-7.28 (m, 3H), 7.27-7.16 (m, 2H), 4.64-4.49 (m, 1H), 4.47-4.32 (m, 1H), 3.62-3.43 (m, 2H), 3.42-3.18 (m, 2H), 3.17-3.02 (m, 2H), 2.40-2.19 (m, 1H), 2.12 (td, J=8.4, 12.0 Hz, 1H), 1.97-1.54 (m, 2H), 1.46-1.39 (m, 9H)

### Step 3: tert-butyl 2-benzyl-4-hydroxy-2,7-diazaspiro[4.4]nonane-7-carboxylate

To a solution of tert-butyl 2-benzyl-4-hydroxy-3-oxo-2,7-diazaspiro[4.4]nonane-7-carboxylate (820 mg, 2.37 mmol, 1 eq) in THF (15 mL) was added borane;methylsulfanylmethane (10 M, 710.12 μL, 3 eq) at 0° C. under N₂. The mixture was stirred at 50° C. for 1 h. LCMS showed starting material was consumed. Several new peaks were shown on LCMS and 47% of desired compound was detected. The reaction mixture was quenched with MeOH (1 mL) and stirred at 80° C. for 12 h. Then the reaction mixture was concentrated under reduced pressure to give a residue. The crude product was purified by reversed-phase HPLC (0.1% FA condition) to give compound tert-butyl 2-benzyl-4-hydroxy-2,7-diazaspiro[4.4]nonane-7-carboxylate (736 mg, 90.16% yield) as white oil.

LC-MS [ESI, M+1]: 333.2.

### Step 4: 2-benzyl-2,7-diazaspiro[4.4]nonan-4-ol

To a solution of tert-butyl 2-benzyl-4-hydroxy-2,7-diazaspiro[4.4]nonane-7-carboxylate (300 mg, 902.43 μmol, 1 eq) in DCM (3 mL) was added TFA (1.54 g, 13.46 mmol, 1.00 mL, 14.92 eq). The mixture was stirred at 25° C. for 1 h. LCMS showed starting material was consumed. Several new peaks were shown on LCMS and 96% of desired compound was detected. The reaction mixture was concentrated under reduced pressure to give compound 2-benzyl-2,7-diazaspiro[4.4]nonan-4-ol (180 mg, 55.57% yield, TFA) as yellow oil.

LC-MS [ESI, M+1]: 233.1.

### Step 5: methyl 6-(2-benzyl-4-hydroxy-2,7-diazaspiro[4.4]nonan-7-yl)pyridine-2-carboxylate

To a solution of 2-benzyl-2,7-diazaspiro[4.4]nonan-4-ol (180 mg, 774.79 μmol, 1 eq) in DMSO (3 mL) was added DIEA (400.54 mg, 3.10 mmol, 539.82 μL, 4 eq) and methyl 6-fluoropyridine-2-carboxylate (120.19 mg, 774.79 μmol, 1 eq). The mixture was stirred at 100° C. for 0.5 h. LCMS showed starting material was consumed. Several new peaks were shown on LCMS and 45% of desired compound was detected. The reaction mixture was diluted with H₂O (5 mL) and extracted with EtOAc (15 mL×3). The combined organic layers were washed with brine (5 mL×3), dried over Na₂SO₄, filtered and concentrated under reduced pressure to give a residue. The residue was purified by prep-TLC (SiO₂, DCM:MeOH=10:1, Rf=0.3) and eluted with DCM and MeOH (10:1, 50 mL) to give the product methyl 6-(2-benzyl-4-hydroxy-2,7-diazaspiro[4.4]nonan-7-yl)pyridine-2-carboxylate (100 mg, 34.70% yield) as white solid.

LC-MS [ESI, M+1]: 368.3.

### Step 6: methyl 6-(9-hydroxy-2,7-diazaspiro[4.4]nonan-2-yl)pyridine-2-carboxylate

To a solution of methyl 6-(2-benzyl-4-hydroxy-2,7-diazaspiro[4.4]nonan-7-yl)pyridine-2-carboxylate (100 mg, 272.15 μmol, 1 eq) in THF (15 mL) was added Pd/C (28.96 mg, 27.22 μmol, 10% purity, 0.1 eq) and Pd(OH)₂ (19.11 mg, 27.22 μmol, 20% purity, 0.1 eq) under N₂. The suspension was degassed and purged with H₂ for 3 times. The mixture was stirred under H₂ (50 psi) at 50° C. for 12 h. LCMS showed starting material was consumed. Several new peaks were shown on LCMS and 49% of desired compound was detected. The reaction mixture was filtered and concentrated under reduced pressure to give a crude methyl 6-(9-hydroxy-2,7-diazaspiro[4.4]nonan-2-yl)pyridine-2-carboxylate (120 mg, 77.91% yield) as white solid.

LC-MS [ESI, M+1]: 278.0.

### Step 7: methyl 6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (20 mg, 59.46 μmol, 1 eq) in DMF (0.5 mL) was added methyl 6-(9-hydroxy-2,7-diazaspiro[4.4]nonan-2-yl)pyridine2-carboxylate (32.98 mg, 118.91 μmol, 2 eq) and DIEA (23.05 mg, 178.37 μmol, 31.07 μL, 3 eq). After addition, the mixture was stirred at 25° C. for 5 min, and then HATU (33.91 mg, 89.18 μmol, 1.5 eq) was added. The resulting mixture was stirred at 25° C. for 25 min. LCMS showed starting material was consumed. Several new peaks were shown on LCMS and 24% of desired compound was detected. The reaction mixture was diluted with H₂O (3 mL) and extracted with EtOAc (5 mL×3). The combined organic layers were washed with brine (3 mL×3), dried over Na₂SO₄, filtered and concentrated under reduced pressure to give a residue. The residue was purified by prep-TLC (SiO₂, DCM:MeOH=10:1, Rf=0.5) and eluted with DCM and MeOH (10:1, 50 mL) to give compound methyl 6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (21 mg, 40.20% yield) as yellow oil.

LC-MS [ESI, M+1]: 596.2.

### Step 8: 6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of methyl 6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (21 mg, 35.25 μmol, 1 eq) in MeOH (0.5 mL) and H₂O (0.1 mL) was added NaOH (2.82 mg, 70.51 μmol, 2 eq). The mixture was stirred at 25° C. for 10 min. LCMS showed starting material was consumed. Several new peaks were shown on LCMS and the 83% of desired compound was detected. The reaction was adjusted pH to 7 with FA (0.1 mL) and then was concentrated under reduced pressure to give a residue. The residue was purified by prep-HPLC (FA condition; column: CD01-Phenomenex Luna C18 150×25 mm×10 um; mobile phase: [H₂O (0.2% FA)—ACN]; gradient: 26%-56% B over 10.0 min) to give compound 6-[4-hydroxy-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (6.23 mg, 30.32% yield) as white solid.

LC-MS [ESI, M+1]: 582.4.

¹H NMR (400 MHz, CHLOROFORM-d) δ=7.77-7.58 (m, 2H), 7.48 (br d, J=5.6 Hz, 1H), 7.34 (td, J=7.2, 18.3 Hz, 4H), 7.21-7.11 (m, 1H), 6.99 (br d, J=5.6 Hz, 1H), 6.71-6.56 (m, 1H), 4.44-4.20 (m, 2H), 4.16-4.03 (m, 1H), 3.99-3.82 (m, 2H), 3.77-3.57 (m, 3H), 3.57-3.46 (m, 3H), 3.45-3.30 (m, 2H), 2.70-2.57 (m, 3H), 2.50 (br s, 1H), 2.41-2.34 (m, 1H), 2.27 (br s, 1H), 2.07 (br d, J=6.0 Hz, 1H), 1.47 (br d, J=7.2 Hz, 3H)

| Compound | | |
|---|---|---|
| Number | Synthesis | LC/MS |
| 703 | Example | m/z 581.68 |
| | 117 | [M + H]⁺ |

### Example 118

### 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinic acid (Compound 704)

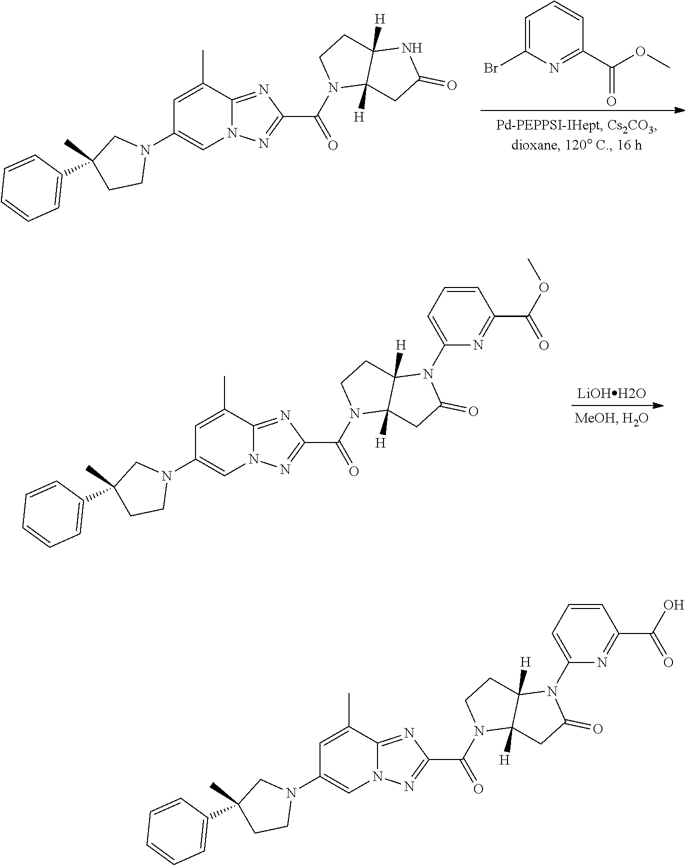 [SMILES: Cc1cc(N2CC[C@](C)(c3ccccc3)C2)cn2nc(C(=O)N3CC[C@@H]4NC(=O)C[C@@H]43)nc12 ; COC(=O)c1cccc(Br)n1 ; COC(=O)c1cccc(N2C(=O)C[C@H]3[C@@H]2CCN3C(=O)c2nc3c(C)cc(N4CC[C@](C)(c5ccccc5)C4)cn3n2)n1 ; Cc1cc(N2CC[C@](C)(c3ccccc3)C2)cn2nc(C(=O)N3CC[C@H]4[C@@H]3CC(=O)N4c3cccc(C(=O)O)n3)nc12]

### Step 1: methyl 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinate

To a solution of (3aS,6aS)-1-[8-methyl-6-[(3R)-3-methyl-3-phenyl-pyrrolidin-1-yl]-[1,2,4]triazolo[1,5-a]pyridine-2-carbonyl]-2,3,3a, 4,6,6a-hexahydropyrrolo[3,2-b]pyrrol-5-one (20 mg, 44.99 μmol, 1 eq) and methyl 6-bromopyridine2-carboxylate (12.64 mg, 58.49 μmol, 1.3 eq) in dioxane (3 mL) was added 1,3-bis[2,6-bis(1-propylbutyl)phenyl]-4,5-dichloro-2H-imidazol-1-ium-2-ide;3-chloropyridine;dichloropalladium (1.31 mg, 1.35 μmol, 0.03 eq) and Cs₂CO₃ (43.98 mg, 134.97 μmol, 3 eq). The mixture was stirred at 120° C. for 1 h. LCMS showed starting material was consumed and the desired mass was detected. The crude product was purified by reversed-phase HPLC (0.1% FA condition) to give compound methyl 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinate (15 mg, 25.88 μmol, 57.52% yield) as a yellow gum.

LC-MS [ESI, M+1]: 580.4

### Step 2: 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinic acid

To a solution of methyl 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinate (15.00 mg, 25.88 μmol, 1 eq) in THF (2 mL) was added LiOH·H₂O (3.26 mg, 77.63 μmol, 3 eq) a. The mixture was stirred at 25° C. for 1 h. The reaction mixture was concentrated under the reduced pressure to give the product. The residue was purified by prep-HPLC (column: CD01-Phenomenex luna C18 150*25*10 um; mobile phase: [H₂O (0.225% FA)—ACN]; gradient:388%-68% B over 10.0 min) and lyophilized to give a residue and then the residue was purified by prep-HPLC (column: CD24-WePure Biotech XPT C18 150*25*7 um; mobile phase: [H₂O (10 mM NH₄HCO₃)—ACN]; gradient:15%-45% B over 13.0 min and lyophilized to give compound 6-((3aS,6aS)-4-(8-methyl-6-((R)-3-methyl-3-phenylpyrrolidin-1-yl)-[1,2,4]triazolo[1,5-a]pyridine-2-carbonyl)-2-oxohexahydropyrrolo[3,2-b]pyrrol-1(2H)-yl)picolinic acid (1.25 mg, 2.21 μmol, 8.54% yield, 100% purity) as a white solid.

LC-MS [ESI, M+1]: 566.2

¹H NMR (CHLOROFORM-d, 400 MHz): δ=8.51-8.74 (m, 1H), 7.97-8.09 (m, 2H), 7.64 (s, 1H), 7.31-7.43 (m, 4H), 7.24-7.27 (m, 1H), 7.00-7.05 (m, 1H), 5.64 (dd, J=8.0, 4.3 Hz, 0.28H), 5.31 (s, 1H), 5.06 (t, J=6.4 Hz, 0.66H), 4.43-4.55 (m, 0.61H), 4.33 (s, 0.32H), 3.98 (d, J=10.4 Hz, 0.64H), 3.66-3.75 (m, 0.3H), 3.61 (d, J=8.4 Hz, 1H), 3.47-3.57 (m, 2H), 3.38-3.45 (m, 1H), 3.19-3.37 (m, 1H), 3.02-3.12 (m, 0.69H), 2.83-2.95 (m, 0.35H), 2.59-2.69 (m, 3H), 2.35-2.47 (m, 2.5H), 2.21-2.32 (m, 1.56H), 1.46-1.49 (m, 3H).

### Example 119

### 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (Compound 705)

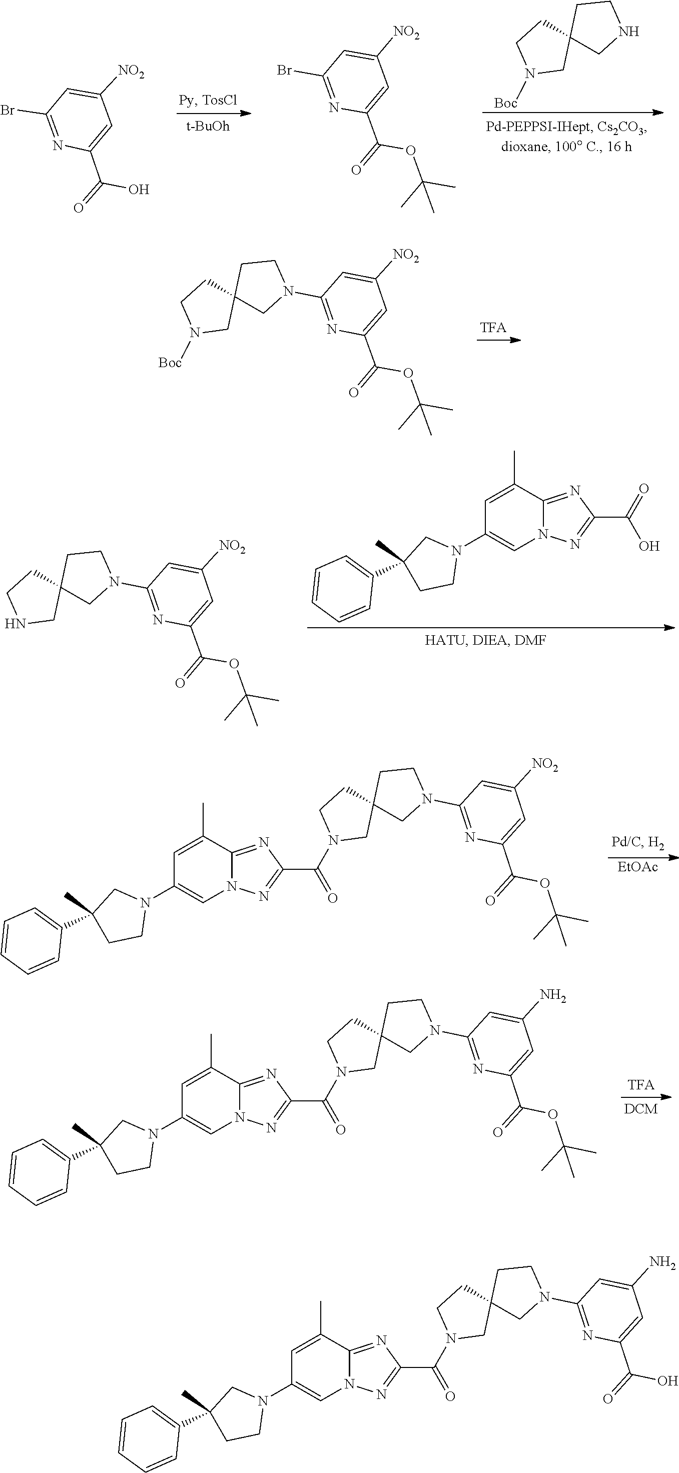

### Step 1: tert-butyl 6-bromo-4-nitro-pyridine-2-carboxylate

To a solution of 6-bromo-4-nitro-pyridine-2-carboxylic acid (5 g, 20.24 mmol, 1 eq) in t-BuOH (50 mL) and Pyridine (12.81 g, 161.94 mmol, 13.07 mL, 8 eq) was added TosCl (11.58 g, 60.73 mmol, 3 eq) at 0° C. The mixture was stirred at 25° C. for 2 h. TLC indicated starting material was consumed and one major new spot with lower polarity was detected. The reaction mixture was quenched with NaHCO₃ (10 mL) and then concentrated under reduced pressure to give a residue. The residue was diluted with H₂O (40 mL) and extracted with EtOAc (50 mL×3). The combined organic layers were washed with brine (20 mL×3), dried over Na₂SO₄, filtered and concentrated under reduced pressure to give a residue. The residue was purified by flash silica gel chromatography (SiO₂, PE:EtOAc=3:1, Rf=0.4) to give compound tert-butyl 6-bromo-4-nitro-pyridine-2-carboxylate (4.54 g, 73% yield) as yellow solid.

LC-MS [ESI, M-55]: 248.0. ¹H NMR (400 MHz, CHLOROFORM-d) δ=8.61 (d, J=1.6 Hz, 1H), 8.35 (d, J=1.6 Hz, 1H), 1.65 (s, 9H)

### Step 2: tert-butyl (5R)-7-(6-tert-butoxycarbonyl-4-nitro-2-pyridyl)-2,7-diazaspiro[4.4]nonane-2-carboxylate

To a solution of tert-butyl 6-bromo-4-nitro-pyridine-2-carboxylate (1.5 g, 4.95 mmol, 1 eq) in dioxane (20 mL) was added tert-butyl (5R)-2,7-diazaspiro[4.4]nonane-2-carboxylate (1.12 g, 4.95 mmol, 1 eq), Cs₂CO₃ (4.03 g, 12.37 mmol, 2.5 eq) and 1,3-bis[2,6-bis(1-propylbutyl)phenyl]-4,5-dichloro-2H-imidazol-1-ium-2-ide;3-chloropyridine;dichloropalladium (481.40 mg, 494.87 μmol, 0.1 eq). The resulting mixture was stirred at 100° C. for 12 h. LCMS showed starting material consumed. One main peak was shown on LCMS and 75% of desired compound was detected. The reaction mixture was filtered and concentrated under reduced pressure to give a residue. The residue was purified by flash silica gel chromatography (SiO₂, PE:EtOAc=3:1, Rf=0.4) to give compound tert-butyl (5R)-7-(6-tert-butoxycarbonyl-4-nitro-2-pyridyl)-2,7-diazaspiro[4.4]nonane-2-carboxylate (1.84 g, 81% yield) as yellow solid.

LC-MS [ESI, M+1]: 449.1.

¹H NMR (400 MHz, CHLOROFORM-d) δ=7.85 (s, 1H), 7.17 (s, 1H), 3.77-3.26 (m, 8H), 2.07-2.03 (m, 2H), 2.01-1.88 (m, 2H), 1.63 (s, 9H), 1.47 (br d, J=5.2 Hz, 9H)

### Step 3: tert-butyl 6-[(5R)-2,7-diazaspiro[4.4]nonan-2-yl]-4-nitro-pyridine-2-carboxylate

To a solution of tert-butyl (5R)-7-(6-tert-butoxycarbonyl-4-nitro-2-pyridyl)-2,7-diazaspiro[4.4]nonane-2-carboxylate (1.84 g, 4.10 mmol, 1 eq) in DCM (18 mL) was added TFA (9.21 g, 80.77 mmol, 6 mL, 19.69 eq). The mixture was stirred at 0° C. for 0.5 h. LCMS showed starting material consumed. One main peak was shown on LCMS and 95% of desired compound was detected. The reaction mixture was adjusted pH to 8 by Na₂CO₃ (10 mL) and diluted with H₂O (20 mL) and extracted with DCM (30 mL×3). The combined organic layers were washed with brine (10 mL×3), dried over Na₂SO₄, filtered and concentrated under reduced pressure to give a residue tert-butyl 6-[(5R)-2,7-diazaspiro[4.4]nonan-2-yl]-4-nitro-pyridine-2-carboxylate (1.2 g, 81% yield) as yellow solid.

LC-MS [ESI, M+1]: 349.1.

### Step 4: tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-4-nitro-pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (1.1 g, 3.27 mmol, 1 eq) in DMF (12 mL) was added tert-butyl 6-[(5R)-2,7-diazaspiro[4.4]nonan-2-yl]-4-nitropyridine-2-carboxylate (1.14 g, 3.27 mmol, 1.00 eq) and HATU (1.87 g, 4.91 mmol, 1.5 eq). After addition, the mixture was stirred at 25° C. for 30 min, and then DIEA (1.27 g, 9.81 mmol, 1.71 mL, 3 eq) was added. The resulting mixture was stirred at 25° C. for 12 h. LCMS showed 4% of starting material remained. Several new peaks were shown on LCMS and 68% of desired compound was detected. The reaction mixture was diluted with H₂O (20 mL) and extracted with EtOAc (30 mL×3). The combined organic layers were washed with brine (10 mL×3), dried over Na₂SO₄, filtered and concentrated under reduced pressure to give a residue. The residue was purified by flash silica gel chromatography (SiO₂, DCM:MeOH=20:1, Rf=0.3) to give compound tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-4-nitro-pyridine-2-carboxylate (1.9 g, 85% yield) as red solid.

LC-MS [ESI, M+1]: 667.5.

¹H NMR (400 MHz, CHLOROFORM-d) δ=7.85 (dd, J=1.2, 4.8 Hz, 1H), 7.65 (br d, J=12.0 Hz, 1H), 7.41-7.28 (m, 4H), 7.27-7.21 (m, 1H), 7.20-7.13 (m, 1H), 7.01 (br d, J=4.0 Hz, 1H), 4.22 (t, J=7.2 Hz, 1H), 4.06-4.01 (m, 1H), 3.95-3.86 (m, 1H), 3.84-3.36 (m, 9H), 2.96 (s, 1H), 2.89 (s, 1H), 2.64 (d, J=7.6 Hz, 3H), 2.43-2.32 (m, 1H), 2.30-2.22 (m, 1H), 2.12-2.07 (m, 2H), 1.63 (d, J=3.2 Hz, 9H), 1.46 (d, J=7.6 Hz, 3H)

### Step 5: tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of tert-butyl 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylate (500 mg, 749.89 μmol, 1 eq) in EtOAc (30 mL) was added Pd/C (79.80 mg, 74.99 μmol, 10% purity, 0.1 eq) under N₂ atmosphere. The suspension was degassed and purged with H₂ for 3 times. The mixture was stirred under H₂ (50 Psi) at 30° C. for 12 h. LCMS showed starting material consumed competely. One main peak was shown on LCMS and 96% of desired compound was detected. The reaction mixture was concentrated under reduced pressure to give a residue tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (450 mg, 91% yield) as red solid.

LC-MS [ESI, M-56+1]: 637.5.

¹H NMR (400 MHz, DMSO-d₆) δ=7.91 (br d, J=11.2 Hz, 1H), 7.46-7.16 (m, 6H), 6.69-6.50 (m, 1H), 5.85 (br d, J=8.0 Hz, 1H), 5.66 (br d, J=16.8 Hz, 1H), 3.93 (br t, J=6.8 Hz, 1H), 3.82-3.73 (m, 1H), 3.66 (br t, J=6.8 Hz, 1H), 3.62-3.46 (m, 4H), 3.46-3.37 (m, 3H), 3.32-3.24 (m, 2H), 2.54 (s, 2H), 2.30-2.18 (m, 2H), 1.99 (s, 3H), 1.96-1.90 (m, 2H), 1.49 (d, J=5.6 Hz, 9H), 1.34 (d, J=8.4 Hz, 3H)

### Step 6: 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (30.00 mg, 47.11 μmol, 1 eq) in DCM (0.5 mL) was added TFA (12 M, 0.5 mL, 127.36 eq). The mixture was stirred at 25° C. for 0.5 h. LCMS showed starting material consumed. Several new peaks were shown on LCMS and 86% of desired compound was detected. The reaction mixture was concentrated under reduced pressure to give a residue. The residue was purified by prep-HPLC (FA condition; column: CD01-Phenomenex luna C18 150×25×10 um; mobile phase: [water(FA)—ACN]; gradient:25%-55% B over 10 min) to give compound 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (16.67 mg, 60.08% yield) as white solid.

LC-MS [ESI, M+1]: 581.3. ¹H NMR (400 MHz, DMSO-d₆) δ=7.90 (br d, J=11.2 Hz, 1H), 7.42-7.19 (m, 6H), 6.80-6.55 (m, 3H), 5.67 (dd, J=1.6, 17.1 Hz, 1H), 3.95 (br t, J=6.8 Hz, 1H), 3.84-3.74 (m, 2H), 3.69-3.63 (m, 2H), 3.62-3.57 (m, 2H), 3.55-3.49 (m, 5H), 2.54 (s, 3H), 2.29-2.22 (m, 2H), 2.07-1.93 (m, 4H), 1.35 (d, J=6.8 Hz, 3H).

| Compound | | |
|---|---|---|
| Number | Synthesis | LC/MS |
| 705 | Example 119 | m/z 580.69 [M + H]+ |

### Example 121

### 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (Compound 706)

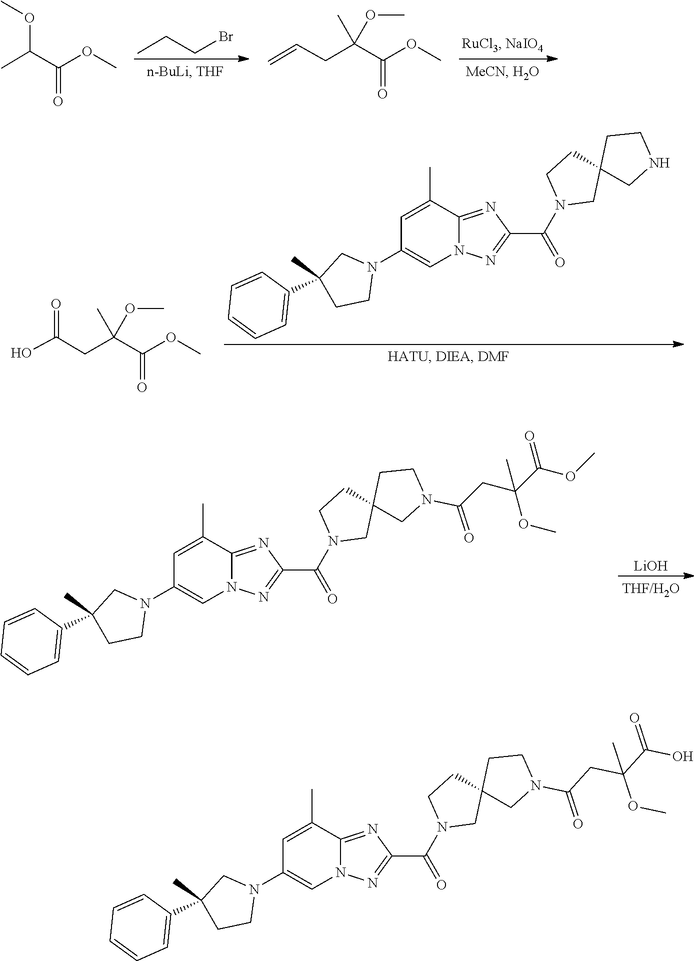

### Step 1: methyl 2-methoxy-2-methyl-pent-4-enoate

To a solution of LDA (2 M, 25.40 mL, 1.2 eq) was added dropwise methyl 2-methoxypropanoate (5 g, 42.33 mmol, 1 eq) in THF (10 mL) at −40° C. over 0.5 h. After stirring for 0.5 h, and then 3-bromoprop-1-ene (6.66 g, 55.02 mmol, 1.3 eq) in THF (10 mL) was added dropwise at −40° C. The resulting mixture was stirred at 0° C. for 1 h. TLC (SiO₂, PE:EtOAc=3:1) indicated reactant was consumed completely and some new spots formed. The reaction mixture quenched with NH₄Cl (20 ml), and extracted with MTBE (20 ml×2). The combined organic layers were washed with brine, dried over Na₂SO₄, filtered, and concentrated in vacuo to give compound methyl 2-methoxy-2-methyl-pent-4-enoate (6 g, 34.14 mmol, 80.65% yield, 90% purity) as yellow oil.

¹H NMR (400 MHz, CHLOROFORM-d) δ=5.82-5.62 (m, 1H), 5.12-4.96 (m, 2H), 3.74-3.64 (m, 3H), 3.25-3.18 (m, 3H), 2.56-2.34 (m, 2H), 1.32 (d, J=2.0 Hz, 3H)

### Step 2: 3,4-dimethoxy-3-methyl-4-oxo-butanoic acid

To a mixture of methyl 2-methoxy-2-methyl-pent-4-enoate (2 g, 12.64 mmol, 1.3 eq) in MeCN (20 mL) and H₂O (20 mL) was added RuCl₃ (100.86 mg, 486.26 μmol, 32.43 μL, 0.05 eq) and NaIO₄ (8.32 g, 38.90 mmol, 2.16 mL, 4 eq) at 0° C. The mixture was stirred at 25° C. for 2 h. TLC (SiO₂, PE:EtOAc=1:1) indicated reactant was consumed completely and some new spots formed. The reaction mixture quenched with Na₂SO₃ (20 ml), and extracted with MTBE (20 ml×2). The combined organic layers were washed with brine, dried over Na₂SO₄, filtered, and concentrated in vacuo to give compound 3,4-dimethoxy-3-methyl-4-oxo-butanoic acid (600 mg, 2.89 mmol, 29.77% yield, 85% purity) as yellow oil.

¹H NMR (400 MHz, CHLOROFORM-d) δ=3.74-3.70 (m, 3H), 3.32-3.28 (m, 3H), 2.84-2.76 (m, 2H), 1.48 (s, 3H)

### Step 3: methyl 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoate

To a mixture of [(5R)-2,7-diazaspiro [4.4]nonan-2-yl]-[8-methyl-6-[(3R)-3-methyl-3-phenyl-pyrrolidin-1-yl]-[1,2,4]triazolo [1,5-a]pyridin-2-yl]methanone (40 mg, 89.97 μmol, 1 eq) in DMF (2 mL) was added HATU (41.05 mg, 107.97 μmol, 1.2 eq) and DIEA (23.26 mg, 179.95 μmol, 31.34 μL, 2 eq). After added 3,4-dimethoxy-3-methyl-4-oxo-butanoic acid (23.78 mg, 134.96 μmol, 1.5 eq). The mixture was stirred at 25° C. for 2 h. LCMS showed starting material was consumed and one main peak with desired mass was detected. The reaction mixture was added water (10 mL) and extracted with EtOAc (10 ml×2). The combined organic layers were washed with brine, dried over Na₂SO₄, filtered, and concentrated in vacuo to give the reside. The residue was purified by prep-HPLC (column: CD24-XPT C18 150*25*7 um; mobile phase: [water(FA)—ACN]; gradient:42%-62% B over 25 min) to give compound methyl 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoate (20 mg, 29.86 μmol, 33.19% yield, 90% purity) as yellow solid.

LC-MS [ESI, M+1]: 603.2

### Step 4: 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid

To a mixture of methyl 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoate (20 mg, 33.18 μmol, 1 eq) in THF (2 mL) and H₂O (0.2 mL) was added LiOH·H₂O (5.57 mg, 132.73 μmol, 4 eq). The mixture was stirred at 25° C. for 2 h. LCMS showed starting material was consumed and the desired mass was detected. The reaction was concentrated under reduced pressure to give a residue. The residue was purified by prep-HPLC (column: CD04-Welch Utimate C18 150*25*7 um; mobile phase: [water(FA)—ACN]; gradient:36%-66% B over 8 min) to give compound 2-methoxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (4.86 mg, 8.26 μmol, 24.88% yield, 100% purity) as white solid.

LC-MS [ESI, M+1]: 589.1

¹H NMR (400 MHz, METHANOL-d4) δ=7.82 (br s, 1H), 7.44-7.32 (m, 4H), 7.30-7.14 (m, 2H), 4.24-4.08 (m, 1H), 4.04-3.93 (m, 1H), 3.92-3.68 (m, 3H), 3.68-3.54 (m, 5H), 3.52-3.40 (m, 3H), 3.28-3.22 (m, 2H), 2.92-2.74 (m, 2H), 2.68-2.56 (m, 3H), 2.44-2.26 (m, 2H), 2.18-1.92 (m, 4H), 1.56-1.42 (m, 5H), 1.56-1.42 (m, 1H)

### Example 122

### 2,2-dimethyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (Compound 707)

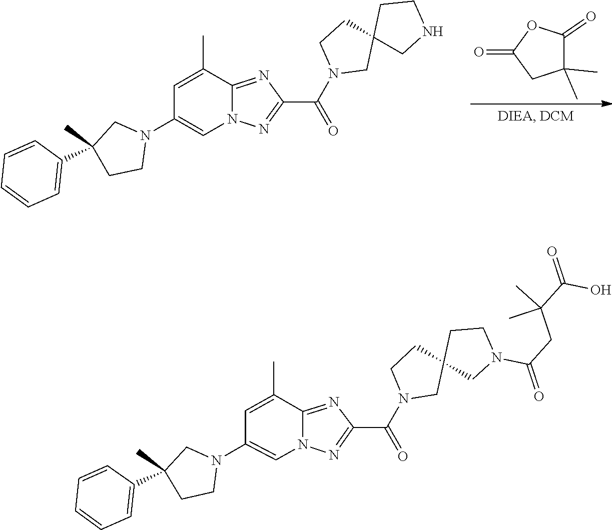

### Step 1: 2,2-dimethyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid

To a solution of [(5R)-2,7-diazaspiro[4.4]nonan-2-yl]-[8-methyl-6-[(3R)-3-methyl-3-phenyl-pyrrolidin-1-yl]-[1,2,4]triazolo[1,5-a]pyridin-2-yl]methanone (40.00 mg, 83.15 μmol, 1 eq, HCl) and 3,3-dimethyltetrahydrofuran-2,5-dione (12.79 mg, 99.79 μmol, 11.26 μL, 1.2 eq) in DMF (1 mL) was added DIEA (32.24 mg, 249.46 μmol, 43.45 μL, 3 eq). The mixture was stirred at 80° C. for 1 h. LCMS showed starting material was consumed and the desired mass was detected. The mixture was quench by H₂O (0.1 ml). The crude product was purified by prep-HPLC (column: CD02-Waters Xbidge BEH C18 150*25*10 um; mobile phase: [water(NH₄HCO₃)—ACN]; gradient:19%-49% B over 10 min) to give compound 2,2-dimethyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (11.32 mg, 24% yield) as white solid.

LC-MS [ESI, M+1]: 573.4.

¹H NMR (400 MHz, CHLOROFORM-d) δ=7.79-7.63 (m, 1H), 7.41-7.28 (m, 4H), 7.27-7.23 (m, 1H), 7.03-6.98 (m, 1H), 4.26-4.08 (m, 1H), 4.06-3.91 (m, 1H), 3.90-3.70 (m, 2H), 3.68-3.58 (m, 3H), 3.58-3.48 (m, 3H), 3.47-3.29 (m, 2H), 2.64 (s, 3H), 2.61-2.57 (m, 1H), 2.41-2.35 (m, 1H), 2.30-2.23 (m, 1H), 2.15-1.88 (m, 5H), 1.47 (s, 3H), 1.41-1.27 (m, 6H).

### Example 123

### (1S,3R)-3-[(5R)-7-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonane-2-carbonyl]cyclopentanecarboxylic acid (Compound 708)

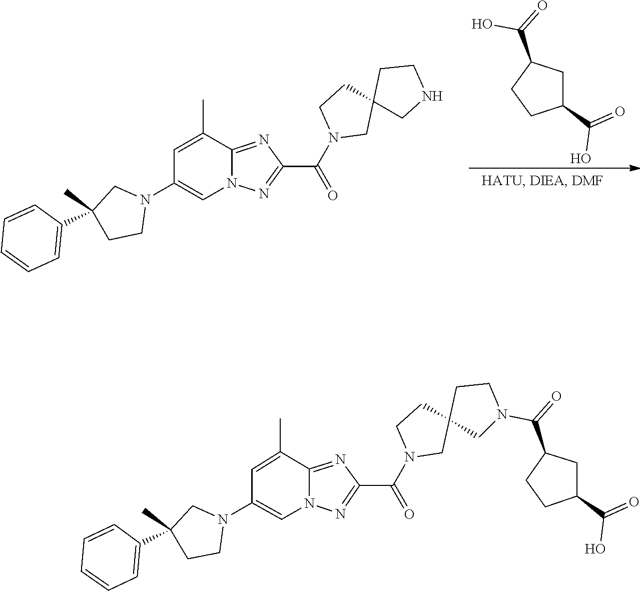

### Step 1: (1S,3R)-3-[(5R)-7-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonane-2-carbonyl]cyclopentanecarboxylic acid

To a solution of [(5R)-2,7-diazaspiro[4.4]nonan-2-yl]-[8-methyl-6-[(3R)-3-methyl-3-phenyl-pyrrolidin-1-yl]-[1,2,4]triazolo[1,5-a]pyridin-2-yl]methanone (40 mg, 83.15 μmol, 1 eq, HCl) and (1R,3S)-cyclopentane-1,3-dicarboxylic acid (15.78 mg, 99.79 μmol, 1.2 eq) in DMF (1 mL) was added DIEA (32.24 mg, 249.46 μmol, 43.45 μL, 3 eq) and HATU (47.43 mg, 124.73 μmol, 1.5 eq). The mixture was stirred at 25° C. for 0.5 h. LCMS showed starting material was consumed and the desired mass was detected. The mixture was quench by H₂O (0.1 ml). The crude product was purified by prep-HPLC (column: CD01-Phenomenex luna C18 150*25*10 um; mobile phase: [water(FA)—ACN]; gradient:45%-75% B over 10 min) to give compound (1S,3R)-3-[(5R)-7-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonane-2-carbonyl]cyclopentanecarboxylic acid (13.88 mg, 28% yield) as white solid.

LC-MS [ESI, M+1]: 585.5.

¹H NMR (400 MHz, CHLOROFORM-d) δ=7.67 (br s, 1H), 7.41-7.29 (m, 4H), 7.25 (br d, J=1.2 Hz, 1H), 7.16-7.03 (m, 1H), 4.29-4.13 (m, 1H), 4.12-3.95 (m, 1H), 3.93-3.82 (m, 1H), 3.74-3.65 (m, 2H), 3.62 (br d, J=8.8 Hz, 2H), 3.58-3.40 (m, 5H), 3.14-2.98 (m, 2H), 2.65 (br d, J=3.6 Hz, 3H), 2.44-2.36 (m, 1H), 2.27 (dt, J=3.6, 7.6 Hz, 2H), 2.20-1.92 (m, 9H), 1.49-1.44 (m, 3H).

| Compound | | |
|---|---|---|
| Number | Synthesis | LC/MS |
| 709 | Similar to Example 123 | m/z 585.3 [M + H]⁺ |
| 710 | Similar to Example 123 | m/z 580.4 [M + H]+ |
| 711 | Similar to Example 123 | m/z 584.3 [M + H]+ |
| 712 | Similar to Example 123 | m/z 583.2 [M + H] |
| 713 | Similar to Example 123 | m/z 597.2 [M + H] |
| 714 | Similar to Example 123 | m/z 566.3 [M + H]⁺ |
| 715 | Similar to Example 123 | m/z 594.3 [M + H]⁺ |
| 716 | Similar to Example 123 | m/z 567.3 [M + H]⁺ |
| 717 | Similar to Example 123 | m/z 566.2 [M + H]⁺ |
| 718 | Similar to Example 123 | m/z 596.3 [M + H]⁺ |
| 719 | Similar to Example 123 | m/z 596.3 [M + H]⁺ |
| 720 | Similar to Example 123 | m/z 571.4 [M + H]⁺ |
| 721 | Similar to Example 123 | m/z 545.5 [M + H]⁺ |
| 722 | Similar to Example 123 | m/z 543.3 [M + H]⁺ |
| 723 | Similar to Example 123 | m/z 596.3 [M + H]⁺ |
| 724 | Similar to Example 123 | m/z 591.3 [M + H]⁺ |
| 725 | Similar to Example 123 | m/z 557.4 [M + H]⁺ |
| 726 | Similar to Example 123 | m/z 599.2 [M + H]⁺ |
| 727 | Similar to Example 123 | m/z 580.29 [M + H]⁺ |
| 728 | Similar to Example 123 | m/z 596.73 [M + H]⁺ |

### Example 124

### 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinic acid (Compound 729)

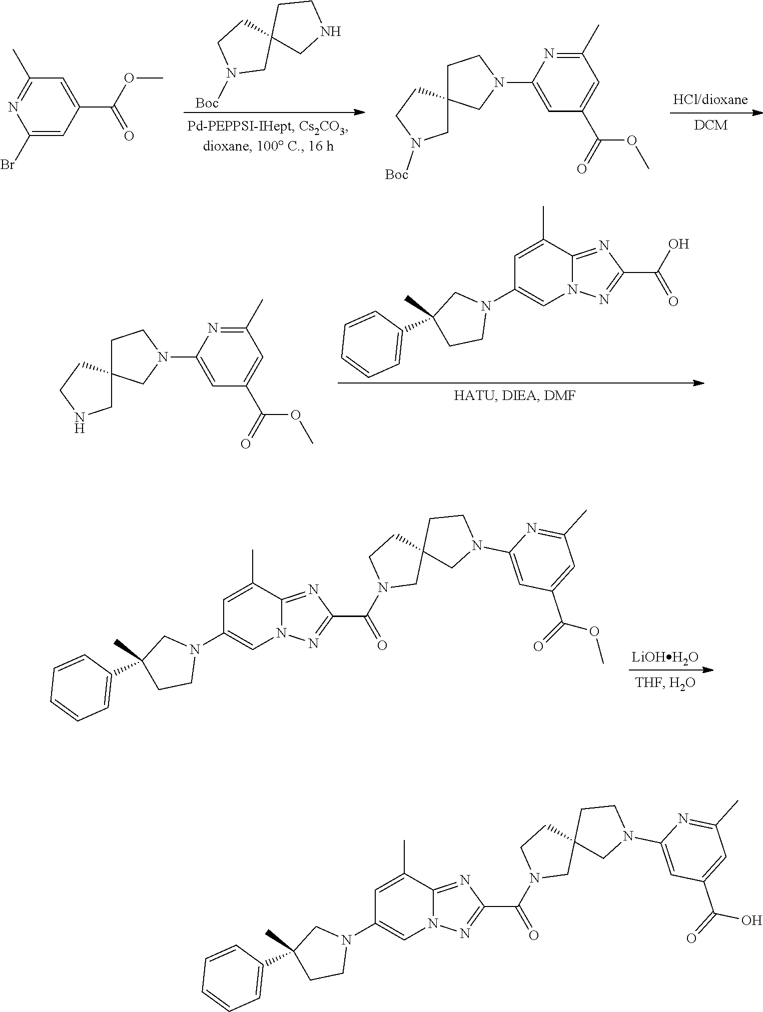

### Step 1: tert-butyl (R)-7-(4-(methoxycarbonyl)-6-methylpyridin-2-yl)-2,7-diazaspiro[4.4]nonane-2-carboxylate

To a solution of methyl 2-bromo-6-methyl-pyridine-4-carboxylate (100 mg, 434.67 μmol, 1 eq) and tert-butyl rac-(5R)-2,7-diazaspiro[4.4]nonane-2-carboxylate (98.37 mg, 434.67 μmol, 1 eq) in dioxane (3 mL) was added Cs₂CO₃ (424.87 mg, 1.30 mmol, 3 eq) and 1,3-bis[2,6-bis(1-propylbutyl)phenyl]-4,5-dichloro-2H-imidazol-1-ium-2-ide;3-chloropyridine;dichloropalladium (42.28 mg, 43.47 μmol, 0.1 eq). The mixture was stirred at 100° C. for 1 h. LCMS showed starting material was consumed and one main peak with desired mass was detected. The reaction mixture was concentrated under the reduced pressure to give the product. The residue was purified by prep-TLC (SiO₂, PE:EtOAc=3:1, Rf=0.4) and eluted with 60 mL (10% methanol in DCM) to give compound tert-butyl (R)-7-(4-(methoxycarbonyl)-6-methylpyridin-2-yl)-2,7-diazaspiro[4.4]nonane-2-carboxylate (100 mg, 266.34 μmol, 61.27% yield) as a white solid.

LC-MS [ESI, M+1]: 376.3.

¹H NMR (CHLOROFORM-d, 400 MHz): δ=6.94 (s, 1H), 6.72 (s, 1H), 3.91 (s, 3H), 3.58 (s, 2H), 3.48 (d, J=10.0 Hz, 4H), 3.24-3.40 (m, 2.3H), 2.45 (s, 3H), 1.93-2.07 (m, 4H), 1.47 (d, J=5.2 Hz, 9H)

### Step 2: methyl (R)-2-methyl-6-(2,7-diazaspiro[4.4]nonan-2-yl)isonicotinate

To a solution of tert-butyl (R)-7-(4-(methoxycarbonyl)-6-methylpyridin-2-yl)-2,7-diazaspiro[4.4]nonane-2-carboxylate (100 mg, 266.34 μmol, 1 eq) in DCM (2 mL) was added HCl/dioxane (2 M, 133.17 μL, 1 eq), The mixture was stirred at 25° C. for 1 h. The reaction mixture was concentrated under the reduced pressure to give compound methyl (R)-2-methyl-6-(2,7-diazaspiro[4.4]nonan-2-yl)isonicotinate (90 mg, crude, HCl) as a brown gum.

LC-MS [ESI, M+1]: 276.1

### Step 3: methyl 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinate

To a solution of methyl (R)-2-methyl-6-(2,7-diazaspiro[4.4]nonan-2-yl)isonicotinate (69.52 mg, 222.96 μmol, 2.5 eq, HCl) and 8-methyl-6-[rac-(3R)-3-methyl-3-phenyl-pyrrolidin-1-yl]-[1,2,4]triazolo[1,5-a]pyridine-2-carboxylic acid (30 mg, 89.18 μmol, 1 eq) in DMF (2 mL) was added HATU (67.82 mg, 178.37 μmol, 2 eq) and DIEA (34.58 mg, 267.55 μmol, 46.60 μL, 3 eq). The mixture was stirred at 25° C. for 1 h. The reaction mixture was added H₂O (5 ml) and then was extracted with EtOAc (3 mL×3), The combined organic layers were washed with brine (3 mL×3), dried over anhydrous Na₂SO₄, filtered and dried to give a residue, The residue was purified by prep-TLC (SiO₂, PE:EtOAc=0:1, Rf=0.3) and eluted with 60 mL (10% methanol in DCM) to give compound methyl 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinate. (52 mg, 87.58 μmol, 98.21% yield) as white solid.

LC-MS [ESI, M+1]: 594.4

### Step 4: 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinic acid

To a solution of methyl 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinate (52 mg, 87.58 μmol, 1 eq) in THF (2 mL) and H₂O (0.3 mL) was added LiOH·H₂O (14.70 mg, 350.34 μmol, 4 eq). The mixture was stirred at 25° C. for hr. The reaction mixture was concentrated under the reduced pressure to give the product. The residue was purified by prep-HPLC (column: CD04-Welch Utimate C18 150*25*7 um; mobile phase: [water(FA)—ACN]; gradient:25%-55% B over 8 min) and lyophilized to give compound 2-methyl-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)isonicotinic acid. as a yellow solid.

LC-MS [ESI, M+1]: 580.4.

¹H NMR (CHLOROFORM-d, 400 MHz): δ=8.22 (s, 1H), 7.65 (d, J=9.2 Hz, 1H), 7.28-7.42 (m, 4H), 7.25 (s, 1H), 6.91-7.05 (m, 2H), 6.83 (s, 1H), 4.18 (s, 1H), 4.00 (d, J=10.8 Hz, 1H), 3.79-3.91 (m, 2H), 3.74 (d, J=12.4 Hz, 2H), 3.57-3.62 (m, 2H), 3.45-3.55 (m, 3H), 3.41 (dd, J=8.0, 3.2 Hz, 1H), 2.62 (br d, J=12.0 Hz, 3H), 2.49 (d, J=4.0 Hz, 3H), 2.36 (d, J=8.4 Hz, 1H), 2.22-2.30 (m, 1H), 1.95-2.19 (m, 4H), 1.46 (d, J=8.4 Hz, 3H)

### Example 125

### 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylic acid (Compound 730)

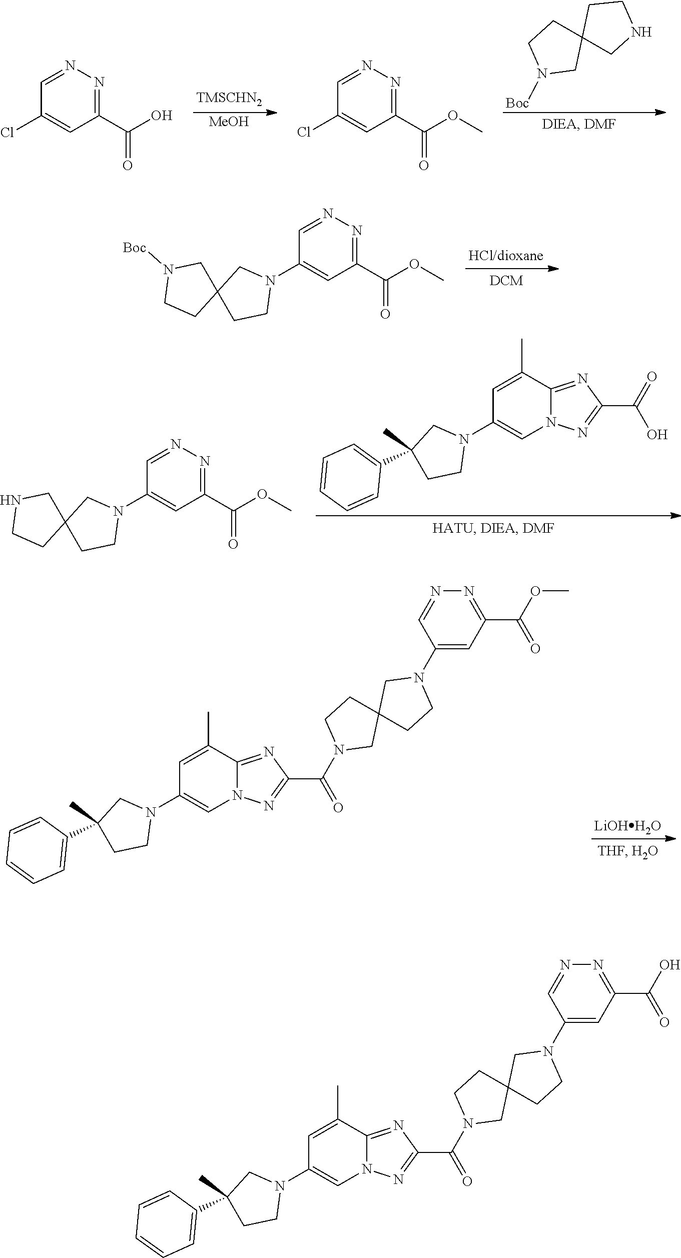

### Step 1: methyl 5-chloropyridazine-3-carboxylate

To a solution of 5-chloropyridazine-3-carboxylic acid (100 mg, 630.75 μmol, 1 eq) in MeOH (3 mL) was added TMSCHN₂ (720.45 mg, 6.31 mmol, 10 eq), the mixture was stirred at 0° C. for 15 min. TLC (PE:EtOAc=0:1, Rf=0.6) indicated reactant was consumed completely and one spot formed. The reaction was clean according to TLC, The reaction mixture was concentrated under the reduced pressure to give compound methyl 5-chloropyridazine-3-carboxylate (100 mg, crude) as a red solid.

### Step 2: tert-butyl 7-(6-(methoxycarbonyl)pyridazin-4-yl)-2,7-diazaspiro [4.4]nonane-2-carboxylate

To a solution of methyl 5-chloropyridazine-3-carboxylate (90 mg, 521.53 μmol, 1 eq) and tert-butyl 2,7-diazaspiro[4.4]nonane-2-carboxylate (118.03 mg, 521.53 μmol, 1 eq) in DMF (3 mL) was added DIEA (337.02 mg, 2.61 mmol, 454.20 μL, 5 eq). The mixture was stirred at 100° C. for 1 h. The reaction mixture was added H₂O (10 ml) and then was extracted with EtOAc (10 mL×3), the combined organic layers were washed with brine (10 ml) dried over anhydrous Na₂SO₄, filtered and dried to give a residue. The residue was purified by prep-TLC (SiO₂, DCM:MeOH (NH₃·MeOH 1%)=20:1, Rf=0.4) and eluted with 60 ml (10% methanol in DCM) to give compound tert-butyl 7-(6-(methoxycarbonyl)pyridazin-4-yl)-2,7-diazaspiro[4.4]nonane-2-carboxylate (95 mg, 262.13 μmol, 50.26% yield) as a yellow solid.

LC-MS [ESI, M+1]: 363.1

### Step 3: methyl 5-(2,7-diazaspiro[4.4]nonan-2-yl)pyridazine-3-carboxylate

To a solution of tert-butyl 7-(6-methoxycarbonylpyridazin-4-yl)-2,7-diazaspiro[4.4]nonane-2-carboxylate (95 mg, 262.13 μmol, 1 eq) in DCM (2 mL) was added HCl/dioxane (2 M, 131.06 μL, 1 eq). The mixture was stirred at 25° C. for 30 min. LCMS showed starting material was consumed and the desired mass was detected. The reaction mixture was concentrated under the reduced pressure to give compound methyl 5-(2,7-diazaspiro[4.4]nonan-2-yl)pyridazine-3-carboxylate (77 mg, crude, HCl) as a white gum.

LC-MS [ESI, M+1]: 263.0

### Step 4: methyl 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylate

To a solution of methyl 5-(2,7-diazaspiro[4.4]nonan-2-yl)pyridazine-3-carboxylate (71.05 mg, 237.82 μmol, 2 eq, HCl) and 8-methyl-6-[rac-(3R)-3-methyl-3-phenyl-pyrrolidin-1-yl]-[1,2,4]triazolo[1,5-a]pyridine-2-carboxylic acid (40 mg, 118.91 μmol, 1 eq) in DMF (2 mL) was added HATU (90.43 mg, 237.82 μmol, 2 eq) and DIEA (46.10 mg, 356.73 μmol, 62.14 μL, 3 eq). The mixture was stirred at 25° C. for 1 h. The reaction mixture was added H₂O (10 ml) and then was extracted with EtOAc (10 mL×3), the combined organic layers were washed with brine (30 mL×3), dried over anhydrous Na₂SO₄, filtered and dried to give a residue. The residue was purified by prep-TLC (SiO₂, DCM:MeOH=20:1, Rf=0.45) and eluted with 60 mL (10% methanol in DCM) to give compound methyl methyl 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylate (30 mg, 51.66 μmol, 43.45% yield) as a white solid.

LC-MS [ESI, M+1]: 581.3.

### Step 5: 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylic acid

To a solution of methyl methyl 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylate (30 mg, 51.66 μmol, 1 eq) in THF (1 mL) and H₂O (0.5 mL) was added LiOH·H₂O (6.50 mg, 154.99 μmol, 3 eq). The mixture was stirred at 25° C. for 1 h. LCMS showed starting material was consumed and the desired mass was detected. The mixture was concentrated to give residue, the residue was adjusted pH around 3-4 by FA, solid was precipitate out, and the mixture was filtered and the solid was concentrated under reduced pressure to give a residue. The residue was purified by prep-HPLC (column: Phenomenex luna C18 150*25 mm*10 um; mobile phase: [water(FA)—ACN]; gradient:28%-58% B over 8 min) and lyophilized to give compound 5-(7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)pyridazine-3-carboxylic acid (14.42 mg, 25.37 μmol, 49.11% yield, 99.712% purity) as a yellow solid.

LC-MS [ESI, M+1]: 567.2.

¹H NMR (METHANOL-d4, 400 MHz): δ=8.56 (s, 1H), 7.74 (s, 1.0H), 7.27-7.39 (m, 5H), 7.07-7.25 (m, 2H), 3.98-4.24 (m, 2H), 3.84 (s, 6H), 3.53 (d, J=16.4 Hz, 3H), 3.32-3.41 (m, 1H), 2.43-2.63 (m, 3H), 2.03-2.38 (m, 6H), 1.40 (d, J=10.0 Hz, 3H).

### Example 126

### 2-hydroxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (Compound 731)

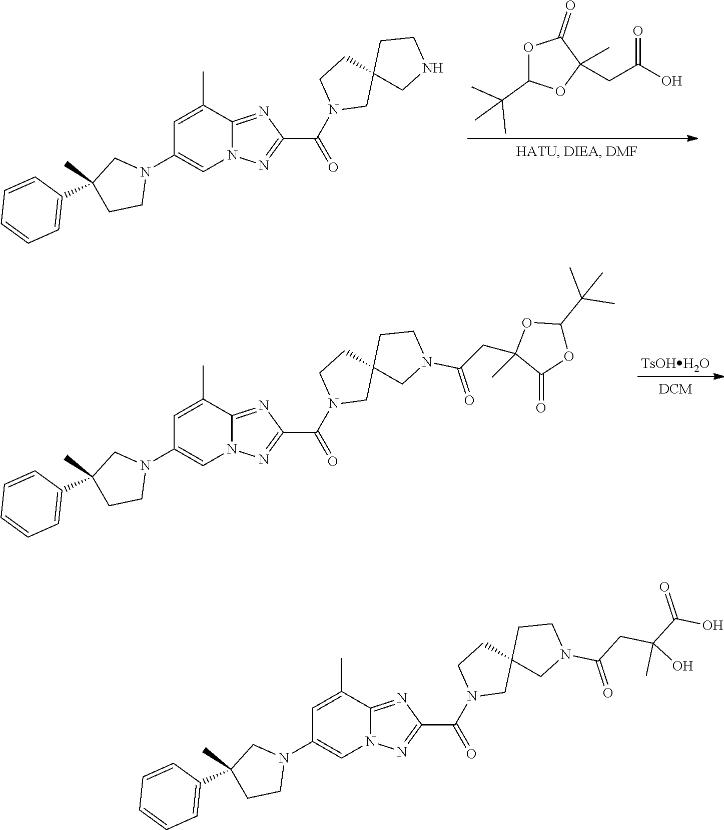

### Step 1: 2-tert-butyl-5-methyl-5-[2-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-2-oxo-ethyl]-1,3-dioxolan-4-one

To a solution of [(5R)-2,7-diazaspiro[4.4]nonan-2-yl]-[8-methyl-6-[(3R)-3-methyl-3-phenyl-pyrrolidin-1-yl]-[1,2,4]triazolo[1,5-a]pyridin-2-yl]methanone (40 mg, 89.97 μmol, 1 eq) and 2-(2-tert-butyl-4-methyl-5-oxo-1,3-dioxolan-4-yl)acetic acid (23.35 mg, 107.97 μmol, 1.2 eq) in DMF (1 mL) was added DIEA (34.89 mg, 269.92 μmol, 47.02 μL, 3 eq) and HATU (41.05 mg, 107.97 μmol, 1.2 eq). The mixture was stirred at 25° C. for 0.3 h. LCMS showed reactant was consumed completely and one main peak with desired mass was detected. The reaction was diluted with H₂O (5 mL) and extracted with EtOAc (10 mL×3). The combined organic layers were washed with brine (10 mL×2), dried over Na₂SO₄, filtered and the filtrate was concentrated under reduced pressure to give a residue. The residue was purified by reversed-phase HPLC (0.1% FA condition) to give 2-tert-butyl-5-methyl-5-[2-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-2-oxo-ethyl]-1,3-dioxolan-4-one (27 mg, 42.00 u mol, 46.69% yield) as a white solid.

LC-MS [ESI, M+1]: 643.3

### Step 2: 2-hydroxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid

To a solution of 2-tert-butyl-5-methyl-5-[2-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-2-oxo-ethyl]-1,3-dioxolan-4-one (22 mg, 34.23 μmol, 1 eq) in DCM (0.8 mL) was added TsOH·H₂O (19.53 mg, 102.68 μmol, 3 eq). The mixture was stirred at 25° C. for 16 h. LCMS showed 23% of reactant remained and 69% of desired mass was detected. The reaction was concentrated under reduced pressure to give a residue. The residue was purified by prep-HPLC (column: CD04-Welch Utimate C18 150×25×7 um; mobile phase: [water(FA)—ACN]; gradient:35%-65% B over 8 min) to give 2-hydroxy-2-methyl-4-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-oxo-butanoic acid (8.01 mg, 13.65 u mol, 39.87% yield, 97.912% purity) as a yellow solid.

LC-MS [ESI, M+1]: 575.4

¹H NMR (400 MHz, DMSO-d₆) δ=7.91 (s, 1H), 7.47 (d, J=8.0 Hz, 0.5H), 7.42-7.28 (m, 5H), 7.26-7.19 (m, 1H), 7.11 (d, J=8.0 Hz, 0.5H), 3.95-3.86 (m, 1H), 3.80-3.67 (m, 2H), 3.64-3.57 (m, 3H), 3.50 (s, 4H), 3.26 (d, J=6.4 Hz, 2H), 2.85-2.72 (m, 1H), 2.58-2.53 (m, 3H), 2.31-2.19 (m, 3H), 1.99-1.79 (m, 4H), 1.38-1.25 (m, 6H).

¹H NMR (400 MHz, METHANOL-d4) δ=7.80 (d, J=5.6 Hz, 1H), 7.70 (d, J=8.0 Hz, 0.5H), 7.42-7.30 (m, 4H), 7.29-7.14 (m, 2.5H), 4.19-3.88 (m, 2H), 3.85-3.76 (m, 1H), 3.75-3.62 (m, 2H), 3.62-3.49 (m, 5H), 3.48-3.37 (m, 2H), 3.08-2.93 (m, 1H), 2.72-2.53 (m, 4H), 2.39-2.34 (m, 1H), 2.33-2.25 (m, 1H), 2.16-1.92 (m, 4H), 1.52-1.36 (m, 6H).

| Compound | | |
|---|---|---|
| Number | Synthesis | LC/MS |
| 732 | Similar to Example 126 | m/z 627.2 [M + H]⁺ |

### Example 127

### 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylic acid (Compound 733)

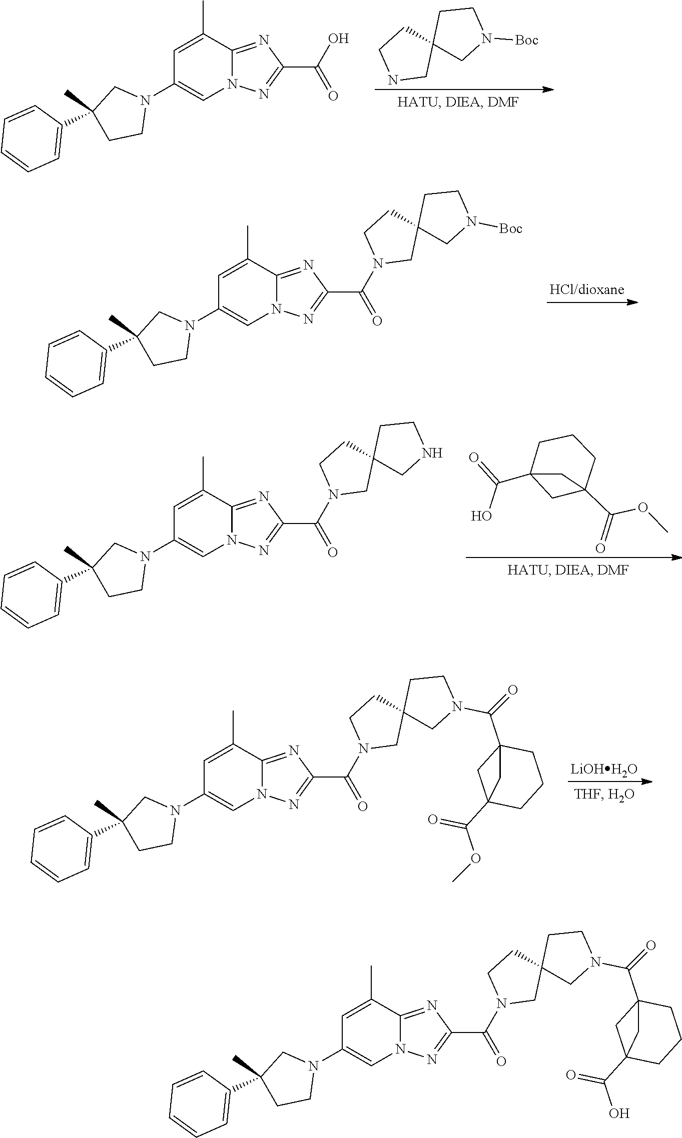

### Step 1: tert-butyl (R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carboxylate

To a solution of tert-butyl (5R)-2,7-diazaspiro[4.4]nonane-2-carboxylate (2.12 g, 9.36 mmol, 1.05 eq) and 8-methyl6-[(3R)-3-methyl-3-phenyl-pyrrolidin-1-yl]-[1,2,4]triazolo[1,5-a]pyridine-2-carboxylic acid (3 g, 8.92 mmol, 1 eq) in DMF (20 mL) was added HATU (10.17 g, 26.75 mmol, 3 eq) and DIEA (5.76 g, 44.59 mmol, 7.77 mL, 5 eq). The mixture was stirred at 25° C. for 1 h. LCMS showed starting material was consumed and the desired mass was detected. The reaction mixture was added H₂O (50 ml) and was extracted with EtOAc (50 mL×3), The combined organic layers were washed with brine (50 mL×3), dried over anhydrous Na₂SO₄, filtered and dried to give a residue. The residue was purified by flash silica gel chromatography (ISCO®; 20 g SepaFlash® Silica Flash Column, Eluent of 0~80% Ethyl acetate/Petroleum ethergradient @40 mL/min) TLC (PE:EtOAc)=0:1, Rf=0.4) to give compound tert-butyl (R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carboxylate (4.86 g, 8.92 mmol, 100.00% yield) as a white solid.

LC-MS [ESI, M+1]: 545.6

¹H NMR (CHLOROFORM-d, 400 MHz): δ=7.66 (s, 1H), 7.29-7.43 (m, 4H), 7.24-7.29 (m, 1H), 7.02 (s, 1H), 3.96 (s, 1H), 3.66-3.91 (m, 3H), 3.59-3.65 (m, 1H), 3.47-3.58 (m, 3H), 3.30-3.45 (m, 3H), 3.22 (dd, J=7.2, 4.4 Hz, 1H), 2.64 (s, 3H), 2.38 (s, 1H), 2.23-2.33 (m, 1H), 1.88-1.96 (m, 4H), 1.45-1.48 (m, 12H)

### Step 2: (8-methyl-6-((R)-3-methyl-3-phenylpyrrolidin-1-yl)-[1,2,4]triazolo[1,5-a]pyridin-2-yl)((R)-2,7-diazaspiro[4.4]nonan-2-yl)methanone

To a solution of tert-butyl (5R)-7-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonane-2-carboxylate (4.86 g, 8.92 mmol, 1 eq) in DCM (50 mL) was added HCl/dioxane (2 M, 4.46 mL, 1 eq).The mixture was stirred at 25° C. for 1 h. LCMS showed starting material was consumed and the desired mass was detected. The reaction mixture was concentrated under the reduced pressure and lyophilized to give compound (8-methyl-6-((R)-3-methyl-3-phenylpyrrolidin-1-yl)-[1,2,4]triazolo[1,5-a]pyridin-2-yl)((R)-2,7-diazaspiro[4.4]nonan-2-yl)methanone (4.29 g, 8.92 mmol, 100.00% yield, HCl) as a yellow gum.

LC-MS [ESI, M+1]: 445.3

### Step 3: methyl 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylate

To a solution of (8-methyl-6-((R)-3-methyl-3-phenylpyrrolidin-1-yl)-[1,2,4]triazolo[1,5-a]pyridin-2-yl)((R)-2,7-diazaspiro[4.4]nonan-2-yl)methanone (40 mg, 89.97 μmol, 1 eq) and 5-methoxycarbonylnorpinane-1-carboxylic acid (35.67 mg, 179.95 μmol, 2 eq) in DMF (3 mL) was added HATU (68.42 mg, 179.95 μmol, 2 eq) and DIEA (34.89 mg, 269.92 μmol, 47.02 μL, 3 eq). The mixture was stirred at 25° C. for 1 h. LCMS showed starting material was consumed and the desired mass was detected. The reaction mixture was added H₂O (10 ml) and was extracted with EtOAc (10 mL×3), The combined organic layers were washed with brine (30 mL×3), dried over anhydrous Na₂SO₄, filtered and dried to give a residue. The residue was purified by prep-TLC (SiO₂, PE:EtOAc=1:1, Rf=0.3) and eluted with 60 mL (10% methanol in DCM) to give compound methyl 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylate (40 mg, 64.02 μmol, 71.16% yield) as a yellow gum.

LC-MS [ESI, M+1]: 625.3

### Step 4: 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylic acid

To a solution of methyl 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylate (40 mg, 64.02 μmol, 1 eq) in THF (3 mL) and H₂O (1 mL) was added LiOH·H₂O (8.06 mg, 192.07 μmol, 3 eq). The mixture was stirred at 25° C. for 1 h. LCMS showed starting material was consumed and the desired mass was detected. The reaction mixture was concentrated under the reduced pressure to give the product. The residue was purified by prep-HPLC (column: CD01-Phenomenex luna C18 150*25*10 um; mobile phase: [water(FA)—ACN]; gradient:35%-65% B over 8 min) and lyophilized to give a residue, and then the reside was purified by prep-HPLC (column: CD24-XPT C18 150*25*7 um; mobile phase: [water(NH₄HCO₃)—ACN]; gradient:22%-52% B over 10 min) and lyophilized to give compound 5-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonane-2-carbonyl)bicyclo[3.1.1]heptane-1-carboxylic acid (10.3 mg, 16.86 μmol, 26.34% yield, 100% purity) as a white solid.

LC-MS [ESI, M+1]: 611.3

¹H NMR (CHLOROFORM-d, 400 MHz): δ=7.67 (s, 1H), 7.29-7.45 (m, 4H), 7.27 (s, 1H), 7.02 (s, 1H), 4.16 (s, 1H), 3.81-4.03 (m, 2H), 3.66-3.80 (m, 1H), 3.28-3.66 (m, 8H), 2.65 (s, 3H), 2.37-2.50 (m, 3H), 2.24-2.32 (m, 1H), 1.79-2.10 (m, 12H), 1.48 (s, 3H).

| Compound Number | Synthesis | LC/MS |
|---|---|---|
| 734 | Similar to Example 127 | m/z 597.3 [M + H]⁺ |

### Example 128

### 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid (Compound 735)

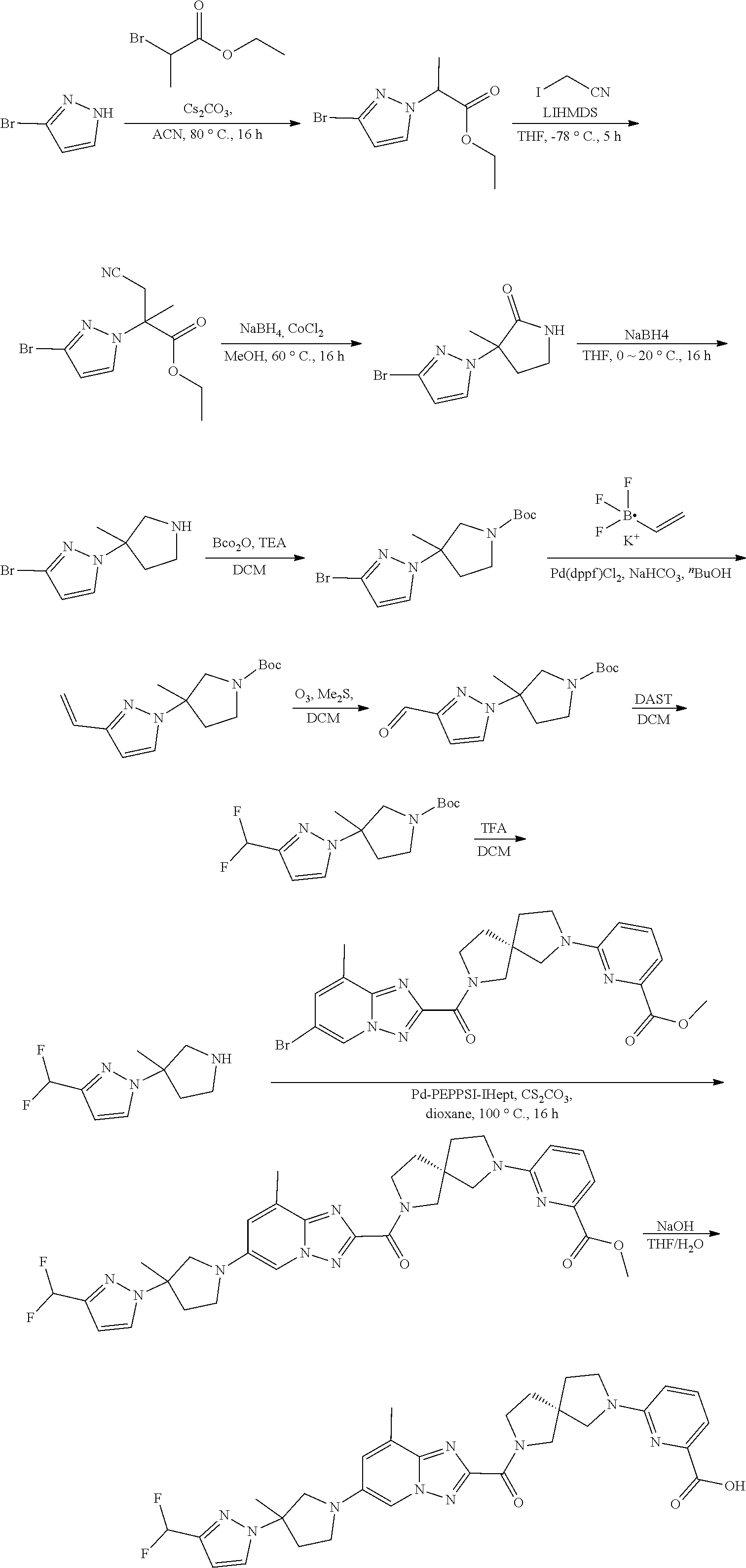

### Step 1: ethyl 2-(3-bromopyrazol-1-yl)propanoate

To a mixture of 3-bromo-1H-pyrazole (20 g, 136.08 mmol, 1 eq), ethyl 2-bromopropanoate (25.13 g, 138.80 mmol, 18.02 mL, 1.02 eq), K₂CO₃ (37.62 g, 272.16 mmol, 2 eq) in ACN (200 mL) was degassed and purged with N₂ for 3 times, and then the mixture was stirred at 60° C. for 2 h under N₂ atmosphere. TLC (SiO₂, PE:EtOAc=3:1) indicated reactant was consumed completely and some new spots formed. The reaction was filtered and the filtered concentrated under reduced pressure to give a residue. The residue was purified by column chromatography (SiO₂, PE:EtOAc=3:1, Rf=0.5) to give compound ethyl 2-(3-bromopyrazol-1-yl) propanoate (33 g, 126.88 mmol, 93.24% yield, 95% purity) as light colorless liquid.

¹H NMR (400 MHz, CHLOROFORM-d) δ=7.46 (d, J=2.4 Hz, 1H), 6.34 (d, J=2.4 Hz, 1H), 5.05 (q, J=7.2 Hz, 1H), 4.21 (q, J=7.2 Hz, 2H), 1.78 (d, J=7.2 Hz, 3H), 1.29-1.25 (m, 3H)

### Step 2: ethyl 2-(3-bromopyrazol-1-yl)-3-cyano-2-methyl-propanoate

To a solution of ethyl 2-(3-bromopyrazol-1-yl)propanoate (33 g, 133.56 mmol, 1 eq) in THF (495 mL) was added LiHMDS (1 M, 200.33 mL, 1.5 eq). The mixture was stirred at −78° C. for 1 h. And then 2-iodoacetonitrile (66.89 g, 400.67 mmol, 3 eq) was added dropwise at −78° C. The resulting mixture was stirred at −78° C. for 1 h under N₂ atmosphere. LCMS showed starting material was consumed and the desired mass was detected. The reaction was quenched by NH₄Cl (200 mL) slowly and then extracted with ethyl acetate (200 mL×3). The combined organic phase was washed with brine (100 mL×3), dried with anhydrous Na₂SO₄, filtered and concentrated under reduced pressure to give a residue. The residue was purified by column chromatography (SiO₂, PE:EtOAc=3:1, Rf=0.3) to give compound ethyl 2-(3-bromopyrazol-1-yl)-3-cyano-2-methyl-propanoate (25 g, 78.64 mmol, 58.88% yield, 90% purity) as colorless liquid.

LC-MS [ESI, M+1]: 287.2.

¹H NMR (400 MHz, CHLOROFORM-d) δ=7.55 (d, J=2.4 Hz, 1H), 6.37 (d, J=2.4 Hz, 1H), 4.29-4.21 (m, 2H), 3.55 (d, J=16.8 Hz, 1H), 3.29 (d, J=16.8 Hz, 1H), 2.02 (s, 3H), 1.27-1.21 (m, 3H)

### Step 3: 3-(3-bromopyrazol-1-yl)-3-methyl-pyrrolidin-2-one

To a solution of ethyl 2-(3-bromopyrazol-1-yl)-3-cyano-2-methyl-propanoate (18 g, 62.91 mmol, 1 eq) in MeOH (900 mL) was added dichlorocobalt (16.34 g, 125.82 mmol, 2 eq) under N₂ atmosphere. The suspension was degassed and purged with N₂ for 3 times. Then NaBH₄ (23.80 g, 629.10 mmol, 10 eq) was added slowly at 0° C. under N₂, the resulting mixture was stirred at 25° C. for 1 h and stirred at 60° C. for 1 h. TLC (SiO₂, PE:EtOAc=0:1) indicated reactant was consumed completely and some new spots formed. The reaction mixture was quenched by addition 1N HCl (30 mL) mL at 0° C., and then the reaction mixture was filtered and then diluted with H₂O (100 mL) and extracted with DCM (100 mL×3). The combined organic layers were washed with brine (50 mL×2), dried over anhydrous sodium sulfate, filtered and concentrated under reduced pressure to give a residue. The residue was purified by column chromatography (SiO₂, PE:EtOAc=3:1, Rf=0.5) to give compound 3-(3-bromopyrazol-1-yl)-3-methyl-pyrrolidin-2-one (6.3 g, 24.52 mmol, 38.98% yield, 95% purity) as colorless oil.

LC-MS [ESI, M+1]: 244.1

¹H NMR (400 MHz, CHLOROFORM-d) δ=7.58 (dd, J=0.8, 2.4 Hz, 1H), 6.24 (d, J=2.4 Hz, 1H), 3.48 (dt, J=5.6, 8.8 Hz, 1H), 3.38-3.26 (m, 1H), 3.08-2.98 (m, 1H), 2.34-2.22 (m, 1H), 1.68 (s, 3H)

### Step 4: 3-bromo-1-(3-methylpyrrolidin-3-yl)pyrazole

To a solution of 3-(3-bromopyrazol-1-yl)-3-methyl-pyrrolidin-2-one (6.3 g, 25.81 mmol, 1 eq) in THF (31.5 mL) was added NaBH₄ (1.95 g, 51.62 mmol, 2 eq) and diethyloxonio(trifluoro) boranuide (9.52 g, 67.11 mmol, 2.6 eq) at 0° C. The mixture was stirred at 65° C. for 1 h. LCMS showed starting material was consumed and the desired mass was detected. The reaction was quenched by NH₄Cl (15 mL) slowly and then extracted with ethyl acetate (15 mL×3). The combined organic phase was washed with brine (10 mL×3), dried with anhydrous Na₂SO₄, filtered, and concentrated under pressure to give the Compound 3-bromo-1-(3-methylpyrrolidin-3-yl)pyrazole (4.5 g, 17.60 mmol, 68.19% yield, 90% purity) as colourless oil.

LC-MS [ESI, M+1]: 231.1

¹H NMR (400 MHz, CHLOROFORM-d) δ=7.54 (d, J=2.4 Hz, 1H), 6.35 (d, J=1.6 Hz, 1H), 4.01 (d, J=12.4 Hz, 1H), 3.82-3.70 (m, 1H), 3.68-3.55 (m, 2H), 2.66-2.54 (m, 1H), 2.40 (td, J=9.2, 13.8 Hz, 1H), 1.85 (s, 3H) Step 5: tert-butyl 3-(3-bromopyrazol-1-yl)-3-methyl-pyrrolidine-1-carboxylate

To a solution of 3-bromo-1-(3-methylpyrrolidin-3-yl)pyrazole (4.1 g, 17.82 mmol, 1 eq) in DCM (20 mL) was added TEA (3.61 g, 35.64 mmol, 4.96 mL, 2 eq) and Boc₂O (5.83 g, 26.73 mmol, 6.14 mL, 1.5 eq). The mixture was stirred at 25° C. for 1 h. TLC (SiO₂, PE:EtOAc=3:1) indicated reactant was consumed completely and some new spots formed. The reaction was filtered and the filtered concentrated under reduced pressure to give a residue. The residue was purified by column chromatography (SiO₂, PE:EtOAc=3:1, Rf=0.4) to give compound tert-butyl 3-(3-bromopyrazol-1-yl)-3-methyl-pyrrolidine-1-carboxylate (4.4 g, 11.99 mmol, 67.30% yield, 90% purity) as colorless liquid.

¹H NMR (400 MHz, CHLOROFORM-d) δ=7.40 (d, J=2.4 Hz, 1H), 6.28 (br s, 1H), 4.12-3.94 (m, 1H), 3.62-3.30 (m, 3H), 2.82-2.58 (m, 1H), 2.12 (td, J=7.6, 12.0 Hz, 1H), 1.62 (d, J=1.2 Hz, 3H), 1.48 (d, J=1.2 Hz, 9H) Step 6: tert-butyl 3-methyl-3-(3-vinylpyrazol-1-yl)pyrrolidine-1-carboxylate

To a mixture of potassium;trifluoro(vinyl)boranuide (1.62 g, 12.11 mmol, 4 eq), tert-butyl 3-(3-bromopyrazol-1-yl)-3-methyl-pyrrolidine-1-carboxylate (1 g, 3.03 mmol, 1 eq), Pd(dppf)C12 (664.74 mg, 908.49 μmol, 0.3 eq), NaHCO₃ (763.19 mg, 9.08 mmol, 353.49 μL, 3 eq) in dioxane (20 mL) and H₂O (5 mL) was degassed and purged with N₂ for 3 times, and then the mixture was stirred at 100° C. for 12 h under N₂ atmosphere. LCMS showed starting material was consumed and the desired mass was detected. The residue was diluted with water (20 mL) and extracted with EtOAc (20 mL×3). The combined organic layers were washed with water (10 mL×2), dried over Na₂SO₄, filtered and concentrated under reduced pressure to give a residue. The residue was purified by column chromatography (SiO₂, PE:EtOAc=3:1, Rf=0.3) to give compound tert-butyl 3-methyl-3-(3-vinylpyrazol-1-yl) pyrrolidine-1-carboxylate (640 mg, 2.19 mmol, 72.39% yield, 95% purity) as colorless oil.

LC-MS [ESI, M+1]: 278.2

¹H NMR (400 MHz, CHLOROFORM-d) δ=7.42 (dd, J=2.4, 11.2 Hz, 1H), 6.74 (dd, J=12.0, 17.6 Hz, 1H), 6.42-6.26 (m, 1H), 5.72 (d, J=17.6 Hz, 1H), 5.36-5.28 (m, 1H), 4.12-3.93 (m, 1H), 3.64-3.30 (m, 3H), 2.79-2.56 (m, 1H), 2.23-2.08 (m, 1H), 1.67 (s, 3H), 1.48 (d, J=1.6 Hz, 9H) Step 7: tert-butyl 3-(3-formylpyrazol-1-yl)-3-methyl-pyrrolidine-1-carboxylate

A stream of OZONE (110.75 mg, 2.31 mmol, 1 eq) is passed through a cooled −78° C. solution of tert-butyl 3-methyl-3-(3-vinylpyrazol-1-yl)pyrrolidine-1-carboxylate (640 mg, 2.31 mmol, 1 eq) in DCM (30 mL) until TLC analysis (EtOAc:PE=5:1) indicates that conversion of starting material (Rf=0.30) to a more polar product is complete. The reaction mixture is then quenched with dimethyl sulfide (716.86 mg, 11.54 mmol, 5 eq) stirred at −20° C. and stirred at 250 for 1 h. TLC (SiO₂, PE:EtOAc=5:1, Rf=0.36) indicated reactant was consumed completely and some new spots formed. The reaction was filtered and the filtered concentrated under reduced pressure to give a residue compound tert-butyl 3-(3-formylpyrazol-1-yl)-3-methyl-pyrrolidine-1-carboxylate (300 mg, 1.02 mmol, 44.22% yield, 95% purity) as colorless oil.

¹H NMR (400 MHz, CHLOROFORM-d) δ=9.90 (s, 1H), 7.48 (d, J=2.4 Hz, 1H), 6.75 (s, 1H), 4.11-4.00 (m, 1H), 3.54-3.24 (m, 3H), 2.72-2.54 (m, 1H), 2.13 (td, J=7.6, 13.0 Hz, 1H), 1.64 (s, 3H), 1.39 (br d, J=4.8 Hz, 9H) Step 8: tert-butyl 3-[3-(difluoromethyl)pyrazol-1-yl]-3-methyl-pyrrolidine-1-carboxylate

To a solution of tert-butyl 3-(3-formylpyrazol-1-yl)-3-methyl-pyrrolidine-1-carboxylate (300 mg, 1.07 mmol, 1 eq) in DCM (2 mL) was added DAST (692.46 mg, 4.30 mmol, 567.59 μL, 4 eq) at 0° C. The mixture was stirred at 25° C. for 1 h. LCMS showed starting material was consumed and the desired mass was detected. The reaction mixture was quenched by addition NaHCO₃ (3 mL) at 0° C., and then diluted with water 10 mL and extracted with DCM (10 mL×2). The combined organic layers were washed with water (5 mL), dried over Na₂SO₄, filtered and concentrated under reduced pressure to give a residue. The crude product was purified by reversed-phase HPLC (0.1% FA condition) to give compound tert-butyl 3-[3-(difluoromethyl) pyrazol-1-yl]-3-methyl-pyrrolidine-1-carboxylate (120 mg, 378.32 μmol, 35.23% yield, 95% purity) as colorless oil.

LC-MS [ESI, M+1]: 302.3

¹H NMR (400 MHz, CHLOROFORM-d) δ=7.54 (d, J=2.4 Hz, 1H), 6.72 (t, J=5.2 Hz, 1H), 6.52 (s, 1H), 4.04 (br dd, J=11.6, 35.1 Hz, 1H), 3.68-3.32 (m, 3H), 2.78-2.58 (m, 1H), 2.18 (td, J=7.6, 12.9 Hz, 1H), 1.68 (s, 3H), 1.48 (s, 9H)

### Step 9: 3-(difluoromethyl)-1-(3-methylpyrrolidin-3-yl)pyrazole

To a solution of tert-butyl 3-[3-(difluoromethyl)pyrazol-1-yl]-3-methyl-pyrrolidine-1-carboxylate (50 mg, 165.93 μmol, 1 eq) was added HCl/dioxane (2 M, 1 mL, 12.05 eq). The mixture was stirred at 25° C. for 1 h. LCMS showed starting material was consumed and the desired mass was detected. The reaction mixture was added 1N Cs₂CO₃ to pH=8 and dried over Na₂SO₄, filtered and concentrated under reduced pressure to give Compound 3-(difluoromethyl)-1-(3-methylpyrrolidin-3-yl) pyrazole (33 mg, 147.60 μmol, 88.95% yield, 90% purity) as yellow oil.

LC-MS [ESI, M+1]: 202.3

### Step 10: methyl 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylate

To a solution of 3-(difluoromethyl)-1-(3-methylpyrrolidin-3-yl)pyrazole (28.00 mg, 139.15 μmol, 1 eq) and methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (34.74 mg, 69.58 μmol, 0.5 eq) and CS₂CO₃ (136.02 mg, 417.46 μmol, 3 eq) in dioxane (2 mL) was added Pd-PEPPSI-IHeptCl (13.54 mg, 13.92 μmol, 0.1 eq). The mixture was stirred at 100° C. for 2 h. LCMS showed starting material was consumed and the desired mass was detected. The reaction mixture was diluted with water (20 mL) and extracted with EtOAc (10 mL×2). The combined organic layers were washed with water (10 mL×2), dried over Na₂SO₄, filtered and concentrated under reduced pressure to give a residue. The residue was purified by prep-TLC (SiO₂, PE:EtOAc=0:1 Rf=0.41) to give Compound methyl 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylate (40 mg, 58.10 μmol, 41.75% yield, 90% purity) as a yellow solid.

LC-MS [ESI, M+1]: 620.5

### Step 11: 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylate (40.00 mg, 64.55 μmol, 1 eq) in THF (2 mL) and H₂O (0.2 mL) was added NaOH (10.33 mg, 258.20 μmol, 4 eq).The mixture was stirred at 25° C. for 2 h. LCMS showed starting material was consumed and the desired mass was detected. The residue was purified by prep-HPLC (column: CD01-Phenomenex luna C18 150*25*10 um; mobile phase: [H₂O (0.225% FA)—ACN]; gradient:21%-51% B over 10.0 min) to give compound 6-[(5R)-2-[6-[3-[3-(difluoromethyl)pyrazol-1-yl]-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylic acid (22.84 mg, 37.61 μmol, 58.27% yield, 99.733% purity) as white solid.

LC-MS [ESI, M+1]: 606.4

¹H NMR (400 MHz, METHANOL-d4) δ=7.88 (dd, J=2.4, 10.8 Hz, 1H), 7.82-7.62 (m, 2H), 7.36 (br dd, J=7.2, 13.8 Hz, 1H), 7.18 (br d, J=16.8 Hz, 1H), 6.58 (d, J=5.6 Hz, 1H), 6.94-6.56 (m, 1H), 6.54-6.46 (m, 1H), 4.24-3.96 (m, 3H), 3.84 (br s, 1H), 3.78-3.54 (m, 6H), 3.54-3.38 (m, 2H), 2.93-2.80 (m, 1H), 2.56 (br d, J=16.4 Hz, 3H), 2.50-2.36 (m, 1H), 2.28-2.02 (m, 4H), 1.76 (d, J=9.6 Hz, 3H).

### Example 129

### 6-[(5R)-2-[6-[(7S)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylic acid (Compound 736)

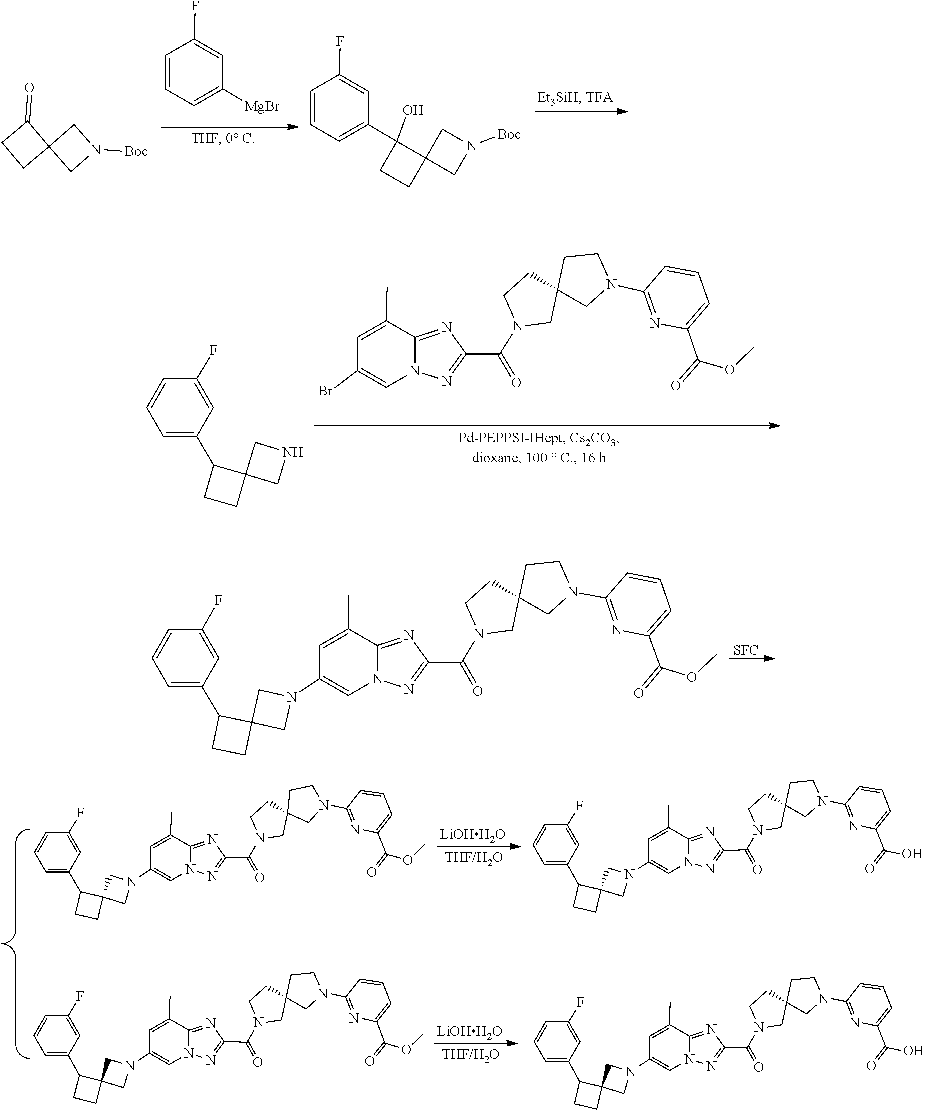

### Step 1: tert-butyl 7-(3-fluorophenyl)-7-hydroxy-2-azaspiro[3.3]heptane-2-carboxylate

A mixture of tert-butyl 7-oxo-2-azaspiro[3.3]heptane-2-carboxylate (700 mg, 3.31 mmol, 1 eq) and bromo-(3-fluorophenyl)magnesium (1 M, 7.62 mL, 2.3 eq) in THF (7 mL) was degassed and purged with N₂ for 3 times, and then the mixture was stirred at 25° C. for 1 h under N₂ atmosphere. TLC showed the starting material was consumed completely. The residue was purified by flash silica gel chromatography (SiO₂, PE:EtOAc=5:1, Rf=0.3) to give compound tert-butyl 7-(3-fluorophenyl)-7-hydroxy-2-azaspiro[3.3]heptane-2-carboxylate (800 mg, 78.55% yield) as a white solid.

¹H NMR (400 MHz, CHLOROFORM-d) δ=7.36 (q, J=7.2 Hz, 1H), 7.24-7.13 (m, 2H), 7.05-6.95 (m, 1H), 4.46 (d, J=9.2 Hz, 1H), 3.77 (br d, J=9.0 Hz, 1H), 3.54-3.39 (m, 2H), 2.64-2.48 (m, 1H), 2.41-2.27 (m, 1H), 2.24-2.13 (m, 1H), 2.11-2.01 (m, 1H), 1.47-1.38 (m, 9H).

### Step 2: 7-(3-fluorophenyl)-2-azaspiro[3.3]heptane

To a solution of tert-butyl 7-(3-fluorophenyl)-7-hydroxy-2-azaspiro[3.3]heptane-2-carboxylate (800 mg, 2.60 mmol, 1 eq) in DCM (8 mL) was added triethylsilane (1.51 g, 13.01 mmol, 2.08 mL, 5 eq), diethyloxonio (trifluoro) boranuide (1.85 g, 13.01 mmol, 1.60 mL, 5 eq) and TFA (2.97 g, 26.03 mmol, 1.93 mL, 10 eq). The mixture was stirred at 80° C. for 2 h. LCMS showed starting material was consumed and the desired mass was detected. The reaction was filtered and the filtered concentrated under reduced pressure to give a residue. The crude product was purified by reversed-phase HPLC (0.1% FA condition) to give compound 7-(3-fluorophenyl)-2-azaspiro[3.3]heptane (450 mg, 90.40% yield) as a white solid.

LC-MS [ESI, M+1]:192.1.

¹H NMR (400 MHz, CHLOROFORM-d) δ=7.42-7.32 (m, 2H), 7.06-6.95 (m, 2H), 6.92-6.84 (m, 1H), 4.60 (br d, J=11.6 Hz, 1H), 4.23 (br d, J=11.6 Hz, 1H), 4.11 (br d, J=10.8 Hz, 1H), 3.97 (br d, J=10.8 Hz, 1H), 3.54 (dt, J=3.2, 8.6 Hz, 1H), 2.42-2.30 (m, 2H), 2.28-2.19 (m, 1H), 2.17-2.07 (m, 1H).

### Step 3: methyl 6-[(5R)-2-[6-[7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate

A mixture of 7-(3-fluorophenyl)-2-azaspiro[3.3]heptane (153.19 mg, 801.03 μmol, 2 eq), methyl 6-[(5R)-2-(6-bromo 8-methyl-[1,2,4]triazolo[1,5-a]pyridine-2-carbonyl)-2,7-diazaspiro[4.4]nonan-7-yl]pyridine-2-carboxylate (200 mg, 400.51 μmol, 1 eq), Cs₂CO₃ (391.48 mg, 1.20 mmol, 3 eq) and 1,3-bis[2,6-bis(1-propylbutyl)phenyl]-4,5-dichloro2H-imidazol-1-ium-2-ide;3-chloropyridine;dichloropalladium (38.96 mg, 40.05 μmol, 0.1 eq) in 1,4-dioxane (3 mL) was degassed and purged with N₂ for 3 times, and then the mixture was stirred at 100° C. for 2 h under N₂ atmosphere. LCMS showed starting material was consumed and the desired mass was detected. The reaction was filtered and the filtered concentrated under reduced pressure to give a residue. The residue was purified by prep-HPLC (column: CD01-Phenomenex luna C18 150*25*10 um; mobile phase: [water(FA)—ACN]; gradient:50%-80% B over 8 min). The result solution was lyophilized to give compound methyl 6-[(5R)-2-[6-[7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate (100 mg, 164.02 μmol, 54.60% yield) as a white solid.

LC-MS [ESI, M+1]:610.3

¹H NMR (400 MHz, CHLOROFORM-d) δ=7.59-7.53 (m, 1H), 7.48-7.41 (m, 1H), 7.40-7.36 (m, 1H), 7.32 (td, J=2.4, 7.8 Hz, 1H), 7.01-6.91 (m, 3H), 6.70-6.61 (m, 1H), 6.58-6.41 (m, 2H), 4.41-4.28 (m, 1H), 4.23-4.11 (m, 2H), 3.93-3.86 (m, 4H), 3.84-3.77 (m, 1H), 3.68-3.56 (m, 7H), 2.56 (d, J=10.0 Hz, 3H), 2.26-2.00 (m, 8H).

### Step 4: methyl 6-[(5R)-2-[6-[(7R)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate and methyl 6-[(5R)-2-[6-[(7S)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate

The residue was purified by prep-HPLC (column: DAICEL CHIRALCEL OD (250 mm*30 mm, 10 um);mobile phase: [C02-ACN/MeOH (0.1% NH₃H₂O)]; B %: 35%, isocratic elution mode). The result solution was lyophilized to give compound methyl 6-[(5R)-2-[6-[(7R)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate (35 mg, 87.50% yield) as a white solid and compound methyl 6-[(5R)-2-[6-[(7S)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate (35 mg, 87.50% yield) as a white solid.

LC-MS [ESI, M+1]: 610.3.

### Step 5:6-[(5R)-2-[6-[(7R)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[6-[(7R)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylate (25.00 mg, 41.00 μmol, 1 eq) in THF (1 mL) and Water (0.5 mL) was added LiOH·H₂O (5.16 mg, 123.01 μmol, 3 eq). The mixture was stirred at 25° C. for 1 h. LCMS showed starting material was consumed and the desired mass was detected. The reaction was filtered and the filtered concentrated under reduced pressure to give a residue. The residue was purified by prep-HPLC (column: Phenomenex Synergi C18 150*25*10 um; mobile phase: [water(0.05% HCl)—ACN]; B %: 55%-75%, 9 min). The result solution was lyophilized to give compound 6-[(5R)-2-[6-[(7R)-7-(3-fluorophenyl)-2-azaspiro[3.3]heptan-2-yl]-8-methyl-[1,2,4]triazolo[1,5-a]pyridine-2-carbonyl]-2,7-diazaspiro[4.4]nonan-7-yl]pyridine-2-carboxylic acid (13.92 mg, 56.00% yield, 98.263% purity) as a white solid.

LC-MS [ESI, M+1]: 596.2

¹H NMR (400 MHz, CHLOROFORM-d) δ=7.65 (ddd, J=5.2, 7.2, 8.4 Hz, 1H), 7.52-7.40 (m, 2H), 7.36-7.28 (m, 1H), 7.06-6.85 (m, 3H), 6.72-6.54 (m, 2H), 4.24-4.08 (m, 1H), 4.01-3.72 (m, 5H), 3.70-3.41 (m, 7H), 2.56 (d, J=11.2 Hz, 3H), 2.46-2.13 (m, 5H), 2.12-1.97 (m, 3H).

| Compound | | |
|---|---|---|
| Number | Synthesis | LC/MS |
| 736 | Example 129 | m/z 596.2 [M + H]⁺ |
| 737 | Similar to Example 129 | m/z596.2[M + H]⁺ |
| 738 | Similar to Example 129 | m/z596.2[M + H]⁺ |
| 739 | Similar to Example 129 | m/z 582.4[M + H]⁺ |
| 740 | Similar to Example 129 | m/z 612.1[M + H]⁺ |
| 741 | Similar to Example 129 | m/z 624.3 [M + H]⁺ |
| 742 | Similar to Example 129 | m/z 624.3 [M + H]⁺ |

### Example 130

### 6-[(5R)-2-[8-methyl-6-[(7S)-7-methyl-7-phenyl-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylic acid (Compound 743)

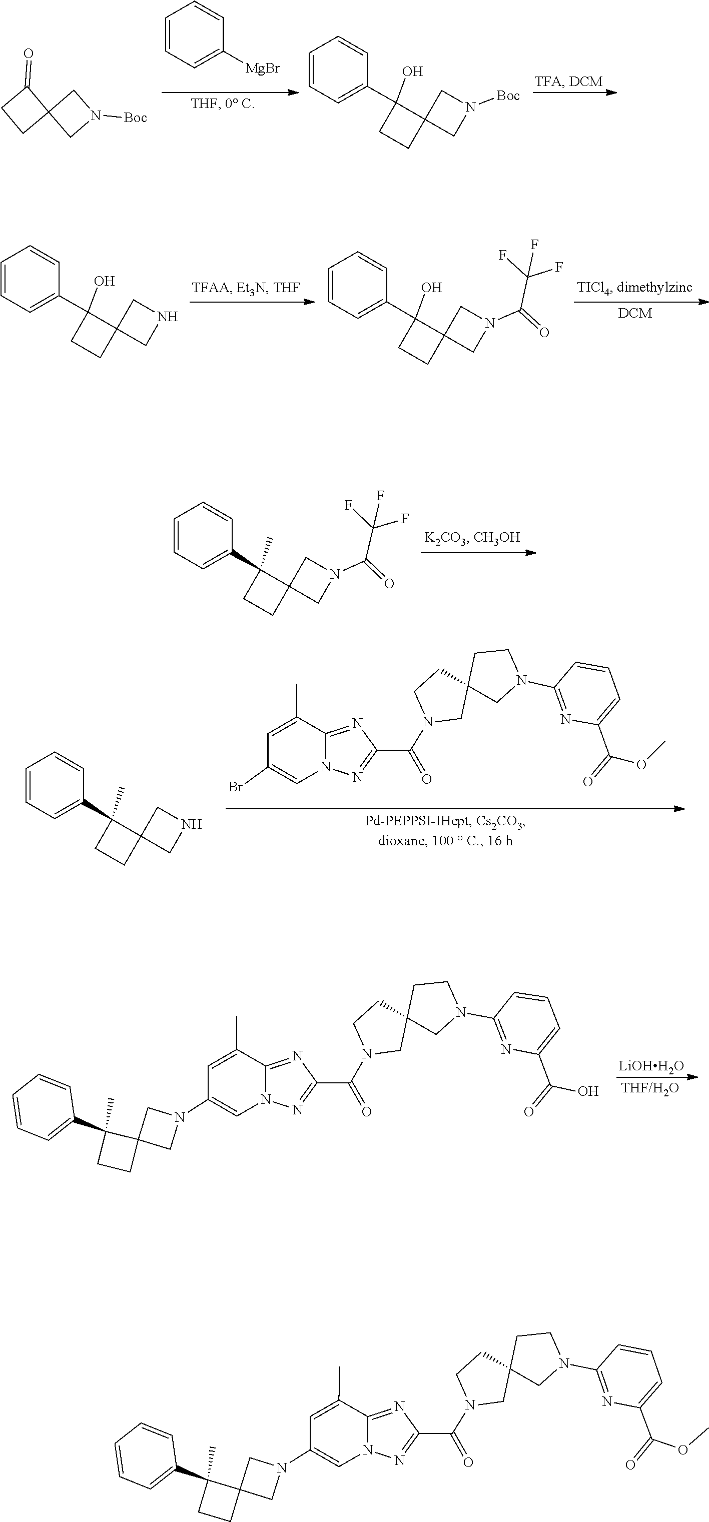

### Step 1: tert-butyl 7-hydroxy-7-phenyl-2-azaspiro[3.3]heptane-2-carboxylate

To a solution of tert-butyl 7-oxo-2-azaspiro [3.3]heptane-2-carboxylate (3.2 g, 15.15 mmol, 1 eq) in THF (30 mL) was added bromo (phenyl) magnesium (1 M, 34.84 mL, 2.3 eq) under N₂. The mixture was stirred at 0° C. for 1 h. LCMS showed starting material was consumed and one main peak with desired mass was detected. The reaction mixture was quenched by addition NH₄Cl solution (100 ml), and then diluted with EtOAc (50 mL) and extracted with EtOAc (50 mL×3). The combined organic layers were dried over Na₂SO₄, filtered and concentrated under reduced pressure to give a residue. The residue was purified by column chromatography (SiO₂, PE:EtOAc=3:1, Rf=0.22) to give compound tert-butyl 7-hydroxy-7-phenyl-2-azaspiro [3.3]heptane-2-carboxylate (3.3 g, 75% yield) as white solid.

LC-MS [ESI, M+23]: 311.9.

¹H NMR (400 MHz, CHLOROFORM-d) δ=7.46-7.37 (m, 4H), 7.32 (br d, J=6.4 Hz, 1H), 4.49 (d, J=9.2 Hz, 1H), 3.78 (d, J=9.2 Hz, 1H), 3.48 (s, 2H), 2.63 (ddd, J=5.6, 9.2, 12.0 Hz, 1H), 2.38-2.29 (m, 1H), 2.21-2.10 (m, 1H), 2.10-2.00 (m, 1H), 1.40 (s, 9H).

### Step 2: 7-phenyl-2-azaspiro[3.3]heptan-7-ol

To a solution of tert-butyl 7-hydroxy-7-phenyl-2-azaspiro [3.3]heptane-2-carboxylate (3.3 g, 11.40 mmol, 1 eq) in trifluoroborane; hydrofluoride (20 mL). The mixture was stirred at 25° C. for 2 h. LCMS showed starting material was consumed and one main peak with desired mass was detected. The mixture was concentrated in vacuo. The residue was purified by column chromatography (SiO₂, DCM:MeOH==4:1, Rf=0.2) to give compound 7-phenyl-2-azaspiro [3.3]heptan-7-ol (2 g, 80% yield) as light yellow oil.

LC-MS [ESI, M+1]: 190.2.

¹H NMR (400 MHz, CHLOROFORM-d) δ=7.47-7.42 (m, 2H), 7.37 (t, J=7.6 Hz, 2H), 7.27 (s, 1H), 4.22 (d, J=8.8 Hz, 1H), 3.53 (d, J=8.8 Hz, 1H), 3.44 (s, 1H), 3.25-3.17 (m, 2H), 2.54-2.43 (m, 1H), 2.26-1.97 (m, 3H).

### Step 3: 2,2,2-trifluoro-1-(7-hydroxy-7-phenyl-2-azaspiro[3.3]heptan-2-yl)ethanone

To a solution of 7-phenyl-2-azaspiro[3.3]heptan-7-ol (2 g, 8.86 mmol, 1 eq, HCl) in THF (20 mL) was added TEA (1.79 g, 17.72 mmol, 2.47 mL, 2 eq) and TFAA (3.72 g, 17.72 mmol, 2.46 mL, 2 eq) at 0° C. under N₂. The mixture was stirred at 0° C. for 1 h. LCMS showed starting material was consumed and one main peak with desired mass was detected. The mixture was concentrated in vacuo. The residue was purified by column chromatography (SiO₂, PE:EtOAc==3:1, Rf=0.42) to give compound 2, 2, 2-trifluoro-1-(7-hydroxy-7-phenyl-2-azaspiro [3.3]heptan-2-yl) ethanone (2.5 g, 79% yield) as colorless oil.

LC-MS [ESI, M+1]: 286.1.

¹H NMR (400 MHz, CHLOROFORM-d) δ=7.47-7.30 (m, 5H), 5.03-4.67 (m, 1H), 4.21 (d, J=10.4 Hz, 1H), 3.95 (d, J=11.2 Hz, 1H), 3.92-3.77 (m, 1H), 3.63 (s, 1H), 2.75-2.63 (m, 1H), 2.43-2.33 (m, 1H), 2.25-2.06 (m, 2H).

### Step 4: (S)-2,2,2-trifluoro-1-(5-methyl-5-phenyl-2-azaspiro[3.3]heptan-2-yl)ethan-1-one

To a solution of 2,2,2-trifluoro-1-(7-hydroxy-7-phenyl-2-azaspiro[3.3]heptan-2-yl) ethanone (2.8 g, 9.82 mmol, 1 eq) in DCM (30 mL) was added tetrachlorotitanium (1 M, 29.45 mL, 3 eq) at −78° C. for 1 h under N₂, followed by dimethylzinc (1 M, 58.89 mL, 6 eq) stirred at −78° C. for 2 h. LCMS showed starting material was consumed and one main peak with desired mass was detected. The mixture was quenched by sat. aq. NaHCO₃ (300 ml) and then diluted with EtOAc (100 mL), the organic layer was dried over Na₂SO₄ and evaporated in vacuo. The residue was purified by column chromatography (PE:EtOAc=4:1) to give compound (S)-2,2,2-trifluoro-1-(5-methyl-5-phenyl-2-azaspiro[3.3]heptan-2-yl)ethan-1-one (300 mg, 10% yield) as yellow oil.

LC-MS [ESI, M+1]: 284.1.

¹H NMR (400 MHz, CHLOROFORM-d) δ=7.46-7.30 (m, 3H), 7.26-7.21 (m, 1H), 7.13 (s, 1H), 4.86-4.56 (m, 1H), 4.53-4.37 (m, 1H), 4.08-3.98 (m, 1H), 3.92-3.75 (m, 1H), 2.51-2.37 (m, 2H), 2.17-2.09 (m, 1H), 2.00-1.87 (m, 1H), 1.50 (d, J=2.0 Hz, 3H).

### Step 5: (7S)-7-methyl-7-phenyl-2-azaspiro[3.3]heptane

To a solution of (S)-2,2,2-trifluoro-1-(5-methyl-5-phenyl-2-azaspiro[3.3]heptan-2-yl)ethan-1-one (300 mg, 10% yield) in MeOH (1 mL) was added K₂CO₃ (73.18 mg, 529.50 μmol, 3 eq). The mixture was stirred at 25° C. for 1 h. LCMS showed starting material was consumed and one main peak with desired mass was detected. The mixture was filtered and concentrated to give compound (7S)-7-methyl-7-phenyl-2-azaspiro [3.3]heptane (33 mg, 95% yield) as yellow solid.

LC-MS [ESI, M+1]: 188.1.

### Step 6: methyl 6-[(5R)-2-[8-methyl-6-[(7S)-7-methyl-7-phenyl-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylate

A mixture of (7S)-7-methyl-7-phenyl-2-azaspiro[3.3]heptane (33 mg, 176.21 μmol, 0.88 eq), methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (100.00 mg, 200.26 μmol, 1 eq), Cs₂CO₃ (195.74 mg, 600.77 μmol, 3 eq) and 1,3-bis[2,6-bis(1-propylbutyl)phenyl]-4,5-dichloro-2H-imidazol-1-ium-2-ide; 3-chloropyridine; dichloropalladium (19.48 mg, 20.03 μmol, 0.1 eq) in dioxane (2 mL) was degassed and purged with N₂ for 3 times, and then the mixture was stirred at 100° C. for 12 h under N₂. LCMS showed starting material was consumed and one main peak with desired mass was detected. The mixture was filtered and concentrated to give a residue. The crude product was purified by reversed-phase HPLC (0.1% FA condition) to give compound methyl 6-[(5R)-2-[8-methyl-6-[(7S)-7-methyl-7-phenyl-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylate (40 mg, 33% yield) as white solid.

LC-MS [ESI, M+1]: 606.2.

### Step 7: 6-[(5R)-2-[8-methyl-6-[(7S)-7-methyl-7-phenyl-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[8-methyl-6-[(7S)-7-methyl-7-phenyl-2-azaspiro[3.3]heptan-2-yl]-[1,2,4]triazolo[1,5-a]pyridine-2-carbonyl]-2,7-diazaspiro[4.4]nonan-7-yl]pyridine-2-carboxylate (20.00 mg, 33.02 μmol, 1 eq) in THF (0.5 mL) and H₂O (0.5 mL) was added LiOH·H₂O (4.16 mg, 99.05 μmol, 3 eq). The mixture was stirred at 25° C. for 1 h. LCMS showed starting material was consumed and one main peak with desired mass was detected. The mixture was filtered and concentrated to give a residue. The crude product was purified by prep-HPLC (column: Phenomenex Luna C18 150*25 mm*10 um; mobile phase: [water(FA)—ACN]; gradient: 31%-61% B over 10 min) to give compound 6-[(5R)-2-[8-methyl-6-[(7S)-7-methyl-7-phenyl-2-azaspiro [3.3]heptan-2-yl]-[1,2,4]triazolo[1,5-a]pyridine-2-carbonyl]-2,7-diazaspiro[4.4]nonan-7-yl]pyridine-2-carboxylic acid (9.54 mg, 49% yield) as white solid.

LC-MS [ESI, M+1]: 592.2.

¹H NMR (400 MHz, CHLOROFORM-d) δ=7.72-7.58 (m, 1H), 7.53-7.40 (m, 2H), 7.36-7.29 (m, 2H), 7.22 (br t, J=6.8 Hz, 3H), 6.70 (br d, J=9.2 Hz, 1H), 6.62 (br t, J=8.8 Hz, 1H), 4.23-4.11 (m, 2H), 3.99 (s, 1H), 3.91 (br t, J=6.8 Hz, 1H), 3.87-3.73 (m, 2H), 3.68-3.46 (m, 6H), 2.58 (d, J=11.2 Hz, 3H), 2.51-2.43 (m, 2H), 2.19-2.04 (m, 5H), 1.98-1.94 (m, 1H), 1.52 (d, J=6.0 Hz, 3H).

### Example 131

### 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylic acid (Compound 744)

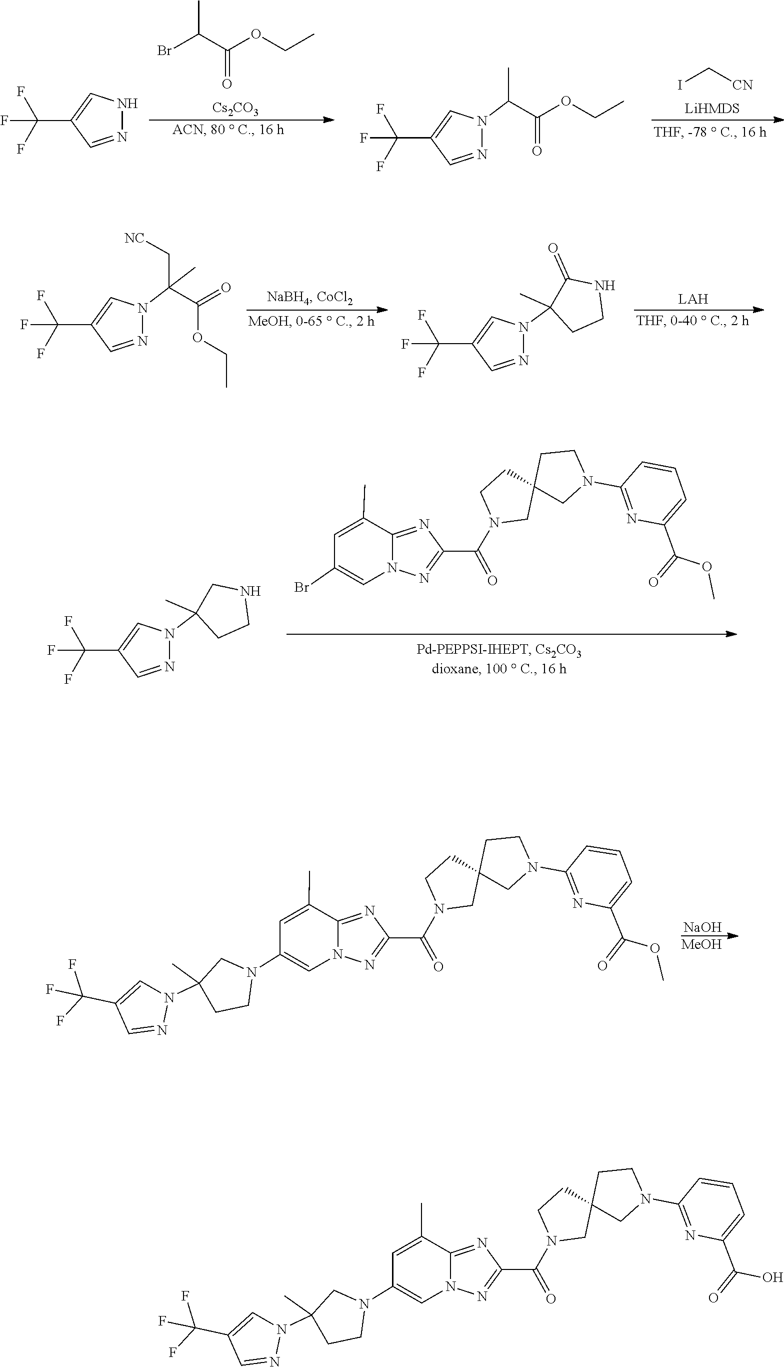

### Step 1: ethyl 2-[4-(trifluoromethyl)pyrazol-1-yl]propanoate

To a solution of 4-(trifluoromethyl)-1H-pyrazole (5 g, 36.74 mmol, 1 eq) and ethyl 2-bromopropanoate (7.32 g, 40.42 mmol, 5.25 mL, 1.1 eq) in ACN (60 mL) was added K₂CO₃ (10.16 g, 73.49 mmol, 2 eq). The mixture was stirred at 60° C. for 2 h. LCMS showed reactant was consumed completely and the desired compound was detected. The reaction mixture was filtered and the filtrated was concentrated in vacuum. The residue was purified by column chromatography (SiO₂, PE:EtOAc=4:1, Rf=0.54) to give compound ethyl 2-[4-(trifluoromethyl)pyrazol-1-yl]propanoate (7.5 g, 86% yield) as a yellow liquid.

LC-MS [ESI, M+1]: 237.1.

¹H NMR (400 MHz, CHLOROFORM-d) δ=7.85 (s, 1H), 7.73 (s, 1H), 5.11 (q, J=7.2 Hz, 1H), 4.23 (q, J=7.2 Hz, 2H), 1.81 (d, J=7.2 Hz, 3H), 1.27 (t, J=7.2 Hz, 3H).

### Step 2: ethyl 3-cyano-2-methyl-2-[4-(trifluoromethyl)pyrazol-1-yl]propanoate

To a solution of ethyl 2-[4-(trifluoromethyl)pyrazol-1-yl]propanoate (7.5 g, 31.75 mmol, 1 eq) in THF (90 mL) was added LiHMDS (1 M, 47.63 mL, 1.5 eq) at −78° C. The mixture was stirred at −78° C. for 1 h under N₂. Then the solution was added 2-iodoacetonitrile (15.90 g, 95.26 mmol, 3 eq). The mixture was stirred at −78° C. for 1 h under N₂. LCMS showed reactant was not consumed completely and the desired mass was detected. The reaction was quenched by NH₄Cl (80 mL) and then extracted with EtOAc (80 mL×3).The combined organic phase was dried over anhydrous Na₂SO₄, filtered and concentrated in vacuum. The residue was purified by column chromatography (SiO₂, PE:EtOAc=3:1, Rf=0.45) to give compound ethyl 3-cyano-2-methyl-2-[4-(trifluoromethyl)pyrazol-1-yl]propanoate (4.8 g, 55% yield) as a yellow liquid.

¹H NMR (400 MHz, CHLOROFORM-d) δ=7.93 (s, 1H), 7.80 (s, 1H), 4.25 (q, J=7.2 Hz, 2H), 3.56 (d, J=17.2 Hz, 1H), 3.31 (d, J=17.2 Hz, 1H), 2.05 (s, 3H), 1.31-1.23 (m, 3H).

LC-MS [ESI, M+1]: 276.2.

### Step 3: 3-methyl-3-[4-(trifluoromethyl)pyrazol-1-yl]pyrrolidin-2-one

To a solution of ethyl 3-cyano-2-methyl-2-[4-(trifluoromethyl)pyrazol-1-yl]propanoate (4.5 g, 16.35 mmol, 1 eq) in MeOH (250 mL) was added dichlorocobalt (4.25 g, 32.70 mmol, 2 eq). Then the solution was added NaBH₄ (6.19 g, 163.50 mmol, 10 eq) at 0° C. under N₂. The mixture was stirred at 65° C. for 2 h under N₂. LCMS showed reactant was consumed completely and the desired mass was detected. The reaction mixture was quenched by HCl (1M) (50 mL). The reaction mixture was filtered and the filtrated was concentrated in vacuum. The residue was purified by column chromatography (SiO₂, PE:EtOAc=1:1, Rf=0.17) to give compound 3-methyl-3-[4-(trifluoromethyl)pyrazol-1-yl]pyrrolidin-2-one (1.1 g, 29% yield) as a yellow solid.

LC-MS [ESI, M+1]: 234.2.

¹H NMR (400 MHz, CHLOROFORM-d) δ=8.05 (s, 1H), 7.75 (s, 1H), 6.77 (s, 1H), 3.57 (dt, J=9.2, 4.8 Hz, 1H), 3.49-3.41 (m, 1H), 3.06 (ddd, J=13.2, 8.0, 6.4 Hz, 1H), 2.42 (ddd, J=13.4, 8.0, 4.8 Hz, 1H), 1.79 (s, 3H).

### Step 4: 1-(3-methylpyrrolidin-3-yl)-4-(trifluoromethyl)pyrazole

To a solution of 3-methyl-3-[4-(trifluoromethyl)pyrazol-1-yl]pyrrolidin-2-one (270 mg, 1.16 mmol, 1 eq) in THF (5 mL) was added LAH (2.5 M, 926.28 μL, 2 eq) at 0° C. The mixture was stirred at 40° C. for 2 h under N₂. LCMS showed reactant was consumed completely and the desired compound was detected. After the reaction mixture was cooled to 0° C., the reaction mixture was quenched by addition of 0.3 mL of H₂O, followed by 0.3 mL of 15% aqueous NaOH and 0.9 mL of H₂O. After being stirred at room temperature for 0.5 h, the solid was removed by filtration. The filtrate was concentrated to dryness to give crude product. The residue was purified by column chromatography (SiO₂, DCM:MeOH=10:1, Rf=0.27) to give compound 1-(3-methylpyrrolidin-3-yl)-4-(trifluoromethyl)pyrazole (135 mg, 53% yield) as a yellow oil.

LC-MS [ESI, M+1]: 220.2.

### Step 5: methyl 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylate

To a solution of methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (50 mg, 100.13 μmol, 1 eq) and 1-(3-methylpyrrolidin-3-yl)-4-(trifluoromethyl)pyrazole (21.95 mg, 100.13 μmol, 1 eq) in dioxane (1 mL) was added [1,3-bis[2,6-bis(1-ethylpropyl)phenyl]-4,5-dichloro-imidazol-2-ylidene]-dichloro-(2-methylpyridin-1-ium-1-yl)palladium (8.41 mg, 10.01 μmol, 0.1 eq) and Cs₂CO₃ (81.56 mg, 250.32 μmol, 2.5 eq). The mixture was stirred at 100° C. for 1 h under N₂. LCMS showed reactant was consumed completely and the desired compound was detected. The reaction mixture was filtered and the filtrated was concentrated in vacuum. The residue was purified by prep-HPLC (column: CD01-Phenomenex luna C18 150*25*10 um;mobile phase: [H₂O (0.1% FA)—ACN]; gradient:35%-45% B over 10.0 min) to give compound methyl 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylate (35 mg, 55% yield) as a yellow solid.

LC-MS [ESI, M+1]: 638.1.

¹H NMR (400 MHz, CHLOROFORM-d) δ=7.82 (d, J=6.0 Hz, 1H), 7.74 (d, J=6.0 Hz, 1H), 7.65 (dd, J=12.4, 1.6 Hz, 1H), 7.59-7.50 (m, 1H), 7.41-7.35 (m, 1H), 6.98-6.91 (m, 1H), 6.58-6.49 (m, 1H), 4.16 (t, J=7.2 Hz, 1H), 4.06-4.01 (m, 1H), 4.00-3.97 (m, 1H), 3.94 (d, J=3.2 Hz, 3H), 3.90 (t, J=7.2 Hz, 1H), 3.82-3.76 (m, 1H), 3.68-3.42 (m, 7H), 2.82 (qd, J=12.4, 6.4 Hz, 1H), 2.63 (d, J=10.4 Hz, 3H), 2.46-2.34 (m, 1H), 2.18-2.01 (m, 4H), 1.81 (d, J=6.4 Hz, 3H).

### Step 6: 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylate (32 mg, 50.18 μmol, 1 eq) in MeOH (1 mL) was added NaOH (4.01 mg, 100.37 μmol, 2 eq) and H₂O (0.2 mL). The mixture was stirred at 25° C. for 0.5 h. LCMS showed reactant was consumed completely and the desired compound was detected. The reaction mixture was added FA until pH around 4. The residue was purified by prep-HPLC (column: CD01-Phenomenex luna C18 150*25*10 um; mobile phase: [H₂O (0.225% FA)—ACN]; gradient:25%-55% B over 10.0 min) to give compound 6-[(5R)-2-[8-methyl-6-[3-methyl-3-[4-(trifluoromethyl)pyrazol-1-yl]pyrrolidin-1-yl]-[1,2,4]triazolo[1,5-a]pyridine-2-carbonyl]-2,7-diazaspiro[4.4]nonan-7-yl]pyridine-2-carboxylic acid (15.68 mg, 50 yield) as a white solid.

LC-MS [ESI, M+1]: 624.2

¹H NMR (400 MHz, METHANOL-d4) δ=8.32 (d, J=8.4 Hz, 1H), 7.85-7.74 (m, 2H), 7.70 (dd, J=13.2, 8.0 Hz, 1H), 7.40-7.28 (m, 1H), 7.25-7.13 (m, 1H), 6.85-6.71 (m, 1H), 4.24-3.95 (m, 3H), 3.85 (s, 1H), 3.79-3.55 (m, 6H), 3.54-3.39 (m, 2H), 2.93-2.79 (m, 1H), 2.65-2.50 (m, 3H), 2.49-2.37 (m, 1H), 2.27-1.99 (m, 4H), 1.76 (d, J=8.4 Hz, 3H).

| Compound | | |
|---|---|---|
| Number | Synthesis | LC/MS |
| 745 | Similar to Example 131 | m/z 570.3 [M + H]⁺ |
| 746 | Similar to Example 131 | m/z 625.3 [M + H]⁺ |
| 747 | Similar to Example 131 | m/z 600.2 [M + H]⁺ |
| 748 | Similar to Example 131 | m/z 618.2 [M + H]⁺ |
| 749 | Similar to Example 131 | m/z 584.2 [M + H] |
| 750 | Similar to Example 131 | m/z 580.2 [M + H]+ |
| 751 | Similar to Example 131 | m/z 618.3 [M + H]+ |

### Example 132

### 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylic acid (Compound 752)

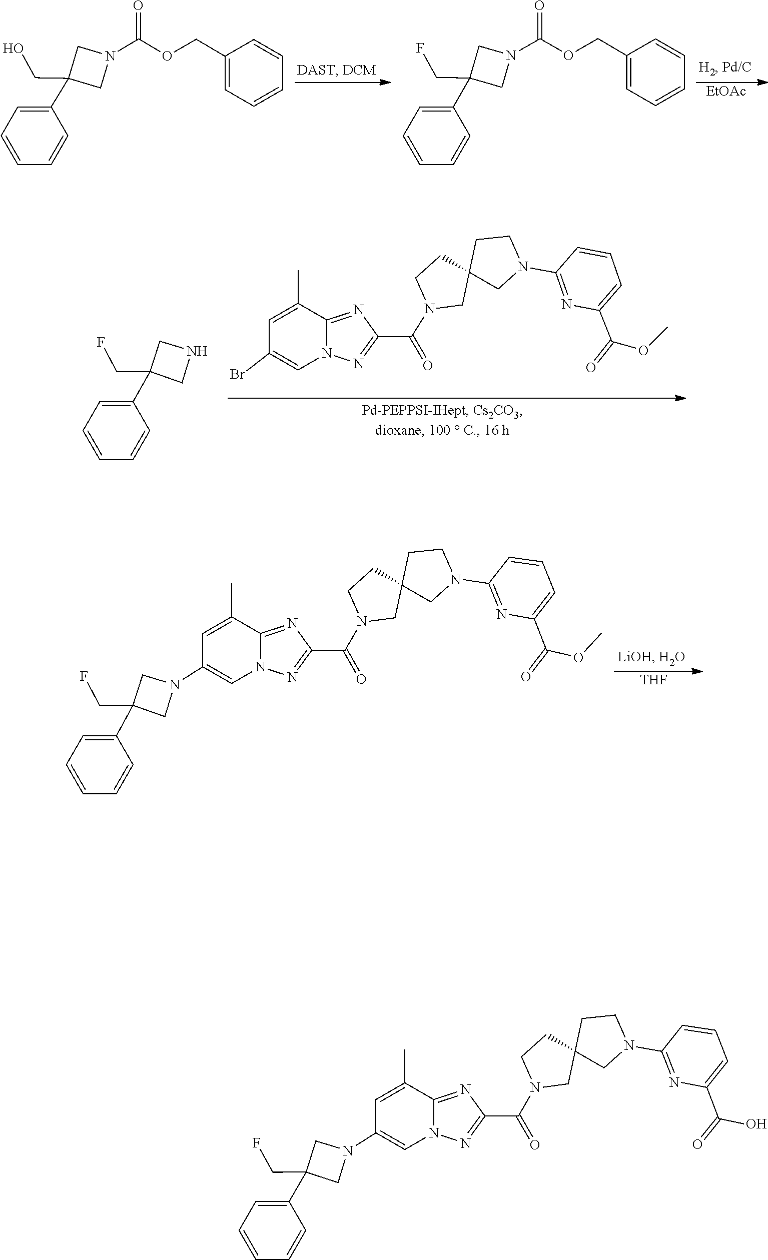

### Step 1: benzyl 3-(fluoromethyl)-3-phenyl-azetidine-1-carboxylate

To a solution of benzyl 3-(hydroxymethyl)-3-phenyl-azetidine-1-carboxylate (380 mg, 1.28 mmol, 1 eq) in Tol. (5 mL) was added 1,1,2,2,3,3,4,4,4-nonafluorobutane-1-sulfonyl fluoride (579.09 mg, 1.92 mmol, 337.47 μL, 1.5 eq) and DBU (583.66 mg, 3.83 mmol, 577.88 μL, 3 eq). The mixture was stirred at 0° C. for 2 h. LCMS showed starting material was consumed and the desired mass was detected. The reaction was filtered and the filtered concentrated under reduced pressure to give a residue. The residue was purified by flash silica gel chromatography (SiO₂, PE:EtOAc=3:1, Rf=0.3) to give compound benzyl 3-(fluoromethyl)-3-phenyl-azetidine-1-carboxylate (290 mg, 75.81% yield) as a white solid.

LC-MS [ESI, M+1]: 300.0.

¹H NMR (400 MHz, CHLOROFORM-d) δ=7.45-7.28 (m, 8H) 7.19-7.11 (m, 2H) 5.12 (s, 2H) 4.31-4.25 (m, 2H) 4.25-4.20 (m, 2H) 3.87 (s, 2H).

### Step 2:3-(fluoromethyl)-3-phenyl-azetidine

To a solution of benzyl 3-(fluoromethyl)-3-phenyl-azetidine-1-carboxylate (200 mg, 668.14 μmol, 1 eq) in EtOAc (5 mL) was added Pd/C (71.10 mg, 66.81 μmol, 10% purity, 0.1 eq) under N₂ atmosphere. The suspension was degassed and purged with H₂ for 3 times. The mixture was stirred under H₂ (15 Psi) at 25° C. for 12 h. LCMS showed starting material was consumed and the desired mass was detected. The reaction was filtered and the filtered concentrated under reduced pressure to give compound 3-(fluoromethyl)-3-phenyl-azetidine (115 mg, crude) as a white solid.

LC-MS [ESI, M+1]:166.0

¹H NMR (400 MHz, CHLOROFORM-d) δ=7.34-7.24 (m, 2H) 7.23-7.15 (m, 1H) 7.05 (d, J=7.4 Hz, 2H) 4.71-4.48 (m, 2H) 3.94 (dd, J=8.4, 2.8 Hz, 2H) 3.77 (d, J=8.4 Hz, 2H).

### Step 3:methyl 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylate

A mixture of 3-(fluoromethyl)-3-phenyl-azetidine (23.65 mg, 143.18 μmol, 1.3 eq), methyl6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (55 mg, 110.14 μmol, 1 eq), CS₂CO₂ (25.82 mg, 330.42 μmol, 3 eq) and 1,3-bis[2,6-bis(1-propylbutyl)phenyl]-4,5-dichloro2H-imidazol-1-ium-2-ide;3-chloropyridine;dichloropalladium (10.71 mg, 11.01 μmol, 0.1 eq) in 1,4-dioxane (2 mL) was degassed and purged with N₂ for 3 times, and then the mixture was stirred at 100° C. for 16 h under N₂ atmosphere. LCMS showed starting material was consumed and the desired mass was detected. The reaction was concentrated under reduced pressure to give a residue. The residue was purified by flash silica gel chromatography (SiO₂, PE:EtOAc=0:1, Rf=0.3) to give compound methyl 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylate (50 mg, crude) as a white solid.

LC-MS [ESI, M+1]: 584.1

### Step 4: 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylate (50 mg, 85.67 μmol, 1 eq) in THF (2 mL) and Water (1 mL) was added LiOH·H₂O (10.78 mg, 257.00 μmol, 3 eq). The mixture was stirred at 25° C. for 1 h. LCMS showed starting material was consumed and the desired mass was detected. The reaction was filtered and the filtered concentrated under reduced pressure to give a residue. The residue was purified by prep-HPLC (column: CD01-Phenomenex luna C18 150*25*10 um; mobile phase: [Water(FA)-MeCN]; gradient:25%-55% B over 10 min). The result solution was lyophilized to give compound 6-[(5R)-2-[6-[3-(fluoromethyl)-3-phenyl-azetidin-1-yl]-8-methyl-[1,2,4]triazolo[1,5-a]pyridine-2-carbonyl]-2,7-diazaspiro[4.4]nonan-7-yl]pyridine-2-carboxylic acid (39.73 mg, 80.57% yield, 98.961% purity) as a white solid.

LC-MS [ESI, M+1]: 570.1

¹H NMR (400 MHz, CHLOROFORM-d) δ=7.70-7.58 (m, 2H) 7.49 (t, J=6.4 Hz, 1H) 7.45-7.37 (m, 2H) 7.36-7.29 (m, 1H) 7.23 (t, J=7.6 Hz, 2H) 6.82 (br d, J=9.6 Hz, 1H) 6.62 (t, J=8.8 Hz, 1H) 4.75 (d, J=8.4 Hz, 1H) 4.63 (d, J=8.4 Hz, 1H) 4.24-4.32 (m, 2H) 4.23-4.11 (m, 3H) 4.00 (s, 1H) 3.91 (t, J=7.2 Hz, 1H) 3.87-3.74 (m, 1H) 3.45-3.71 (m, 4H) 2.62 (d, J=11.4 Hz, 3H) 2.23-2.01 (m, 4H).

| Compound | | |
|---|---|---|
| Number | Synthesis | LC/MS |
| 752 | Example 132 | m/z 570.1 [M + H]⁺ |

### Example 133

### 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylic acid (Compound 753)

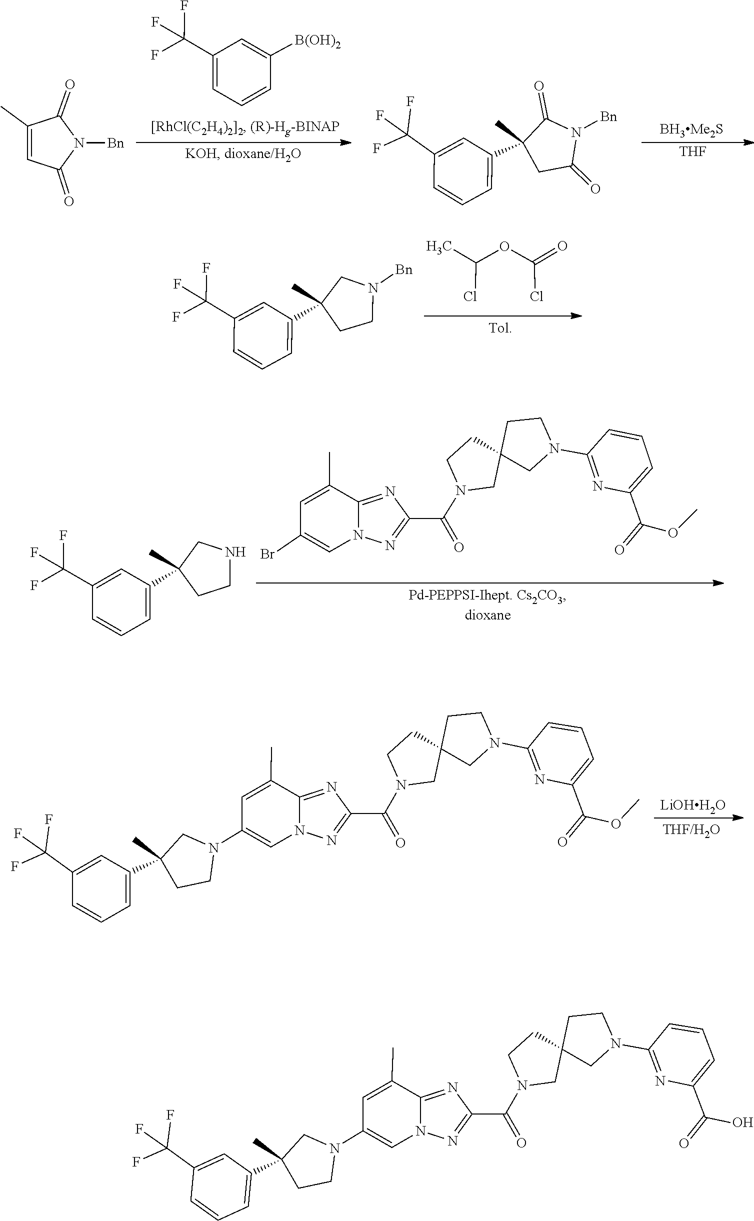

### Step 1: (3R)-1-benzyl-3-methyl-3-[3-(trifluoromethyl)phenyl]pyrrolidine-2,5-dione

A mixture of chlororhodium;ethylene (38.66 mg, 99.39 μmol, 0.02 eq) and BINAP (61.89 mg, 99.39 μmol, 0.02 eq) in dioxane (20 mL) was degassed and purged with N₂ for 3 times, and then the mixture was stirred at 25° C. for 15 min under N₂ atmosphere. Then [3-(trifluoromethyl)phenyl]boronic acid (2.83 g, 14.91 mmol, 3 eq) and KOH (1 M, 2.48 mL, 0.5 eq) was added and degassed and purged with N₂ for 3 times. The mixture was stirred at 25° C. for 5 min under N₂ atmosphere. Then 1-benzyl-3-methyl-pyrrole-2,5-dione (1 g, 4.97 mmol, 1 eq) in dioxane (40 mL) was added and degassed and purged with N₂ for 3 times. The mixture was stirred at 50° C. for 160 min under N₂ atmosphere. LCMS showed starting material was consumed. Several new peaks were shown on LCMS and 14% of desired compound was detected. The reaction mixture was diluted with H₂O (10 mL) and extracted with EtOAc (15 mL×3). The combined organic layers were washed with brine (5 mL×3), dried over Na₂SO₄, filtered and concentrated under reduced pressure to give a residue. The residue was purified by flash silica gel chromatography (SiO₂, PE:EtOAc=4:1, Rf=0.3) to compound (3R)-1-benzyl-3-methyl-3-[3-(trifluoromethyl)phenyl]pyrrolidine-2,5-dione (707 mg, 40.67% yield) as white oil.

LC-MS [ESI, M+1]: 348.0.

¹H NMR (400 MHz, CHLOROFORM-d) δ=7.62-7.51 (m, 2H), 7.51-7.43 (m, 2H), 7.41-7.36 (m, 2H), 7.36-7.29 (m, 3H), 4.79-4.71 (m, 2H), 3.16-3.01 (m, 1H), 2.99-2.84 (m, 1H), 1.73 (s, 3H).

### Step 2: (3R)-1-benzyl-3-methyl-3-[3-(trifluoromethyl)phenyl]pyrrolidine

To a solution of (3R)-1-benzyl-3-methyl-3-[3-(trifluoromethyl)phenyl]pyrrolidine-2,5-dione (600 mg, 1.73 mmol, 28.79 μL, 1 eq) in THF (15 mL) was added borane;methylsulfanylmethane (10 M, 1.73 mL, 10 eq) at 0° C. The mixture was stirred at 70° C. for 1 h. LCMS showed starting material was consumed. Several new peaks were shown on LCMS and 37% of desired compound was detected. The reaction mixture was quenched with MeOH (1 mL) and stirred at 80° C. for 12 h. Then the reaction mixture was diluted with H₂O (5 mL) and extracted with EtOAc (15 mL×3). The combined organic layers were washed with brine (3 mL×3), dried over Na₂SO₄, filtered and concentrated under reduced pressure to give a residue. The residue was purified by flash silica gel chromatography (SiO₂, PE:EtOAc=4:1, Rf=0.3) to give compound (3R)-1-benzyl-3-methyl-3-[3-(trifluoromethyl)phenyl]pyrrolidine (385 mg, 68.95% yield) as yellow oil.

LC-MS [ESI, M+1]: 320.2.

¹H NMR (400 MHz, CHLOROFORM-d) δ=7.62 (s, 1H), 7.53 (br d, J=7.2 Hz, 1H), 7.49-7.30 (m, 6H), 7.29-7.24 (m, 1H), 3.78-3.61 (m, 2H), 2.93-2.80 (m, 2H), 2.79-2.67 (m, 2H), 2.20 (ddd, J=6.8, 8.6, 12.7 Hz, 1H), 2.02 (ddd, J=5.2, 7.9, 12.8 Hz, 1H), 1.48 (s, 3H).

### Step 3: (3R)-3-methyl-3-[3-(trifluoromethyl)phenyl]pyrrolidine

To a solution of (3R)-1-benzyl-3-methyl-3-[3-(trifluoromethyl)phenyl]pyrrolidine (250 mg, 782.81 μmol, 1 eq) in toluene (3 mL) was added 1-chloroethyl carbonochloridate (447.67 mg, 3.13 mmol, 4 eq). The mixture was stirred at 100° C. for 12 h. LCMS showed starting material was consumed. Several new peaks were shown on LCMS and 12% of desired compound was detected. The reaction mixture was quenched with MeOH (5 mL) and stirred at 70° C. for 1 h. Then the mixture was diluted with H₂O (10 mL) and extracted with EtOAc (15 mL×3). The combined organic layers were washed with brine (5 mL×3), dried over Na₂SO₄, filtered and concentrated under reduced pressure to give a residue. The residue was purified by prep-TLC (SiO₂, DCM:MeOH=10:1, Rf=0.2) and eluted with DCM and MeOH (20:1, 50 mL) to give the product (3R)-3-methyl-3-[3-(trifluoromethyl)phenyl]pyrrolidine (70 mg, 39.01% yield) as yellow oil.

LC-MS [ESI, M+1]: 230.1.

¹H NMR (400 MHz, CHLOROFORM-d) δ=7.60-7.42 (m, 4H), 3.61-3.41 (m, 4H), 2.41-2.18 (m, 2H), 1.52 (s, 3H).

### Step 4: methyl 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylate

To a solution of methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (70 mg, 140.18 μmol, 1 eq) in dioxane (2 mL) was added (3R)-3-methyl-3-[3-(trifluoromethyl)phenyl]pyrrolidine (32.13 mg, 140.18 μmol, 1 eq), Cs₂CO₃ (114.18 mg, 350.45 μmol, 2.5 eq) and 1,3-bis[2,6-bis(1-propylbutyl)phenyl]-4,5-dichloro-2H-imidazol-1-ium-2-ide;3-chloropyridine;dichloropalladium (13.64 mg, 14.02 μmol, 0.1 eq) under N₂ atmosphere. The suspension was degassed and purged with N₂ for 3 times. The mixture was stirred under N₂ at 100° C. for 12 h. LCMS showed starting material was consumed. Several new peaks were shown on LCMS and 65% of desired compound was detected. The reaction mixture was concentrated under reduced pressure to give a residue. The residue was purified by prep-TLC (SiO₂, PE:EtOAc=3:1, Rf=0.3) and eluted with EtOAc (50 mL) to give the product methyl 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylate (50 mg, 43.45% yield) as yellow oil.

LC-MS [ESI, M+1]: 648.4.

### Step 5: 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylate (50 mg, 77.20 μmol, 1 eq) in THF (0.4 mL) and H₂O (0.1 mL) was added LiOH·H₂O (12.96 mg, 308.79 μmol, 4 eq). The mixture was stirred at 25° C. for 0.5 h. LCMS showed starting material was consumed. Several new peaks were shown on LCMS and 34% of desired compound was detected. The reaction mixture was concentrated under reduced pressure to give a residue. The residue was purified by prep-HPLC (neutral condition; column: CD02-Waters Xbidge BEH C18 150×25×10 um; mobile phase: [water(NH₄HCO₃)—ACN]; gradient: 23%-53% B over 10 min) to give compound 6-[(5R)-2-[8-methyl-6-[(3R)-3-methyl-3-[3-(trifluoromethyl)phenyl]pyrrolidin-1-yl]-[1,2,4]triazolo[1,5-a]pyridine-2-carbonyl]-2,7-diazaspiro[4.4]nonan-7-yl]pyridine-2-carboxylic acid (12.28 mg, 25.08% yield) as white solid.

LC-MS [ESI, M+1]: 634.4.

¹H NMR (400 MHz, CHLOROFORM-d) δ=7.72-7.61 (m, 2H), 7.61-7.42 (m, 5H), 7.27 (s, 1H), 7.01 (br d, J=9.6 Hz, 1H), 6.62 (t, J=8.4 Hz, 1H), 4.33-4.11 (m, 1H), 4.02 (s, 1H), 3.96-3.74 (m, 2H), 3.72-3.31 (m, 8H), 2.65 (d, J=11.2 Hz, 3H), 2.45-2.27 (m, 2H), 2.22-2.02 (m, 4H), 1.49 (d, J=7.6 Hz, 3H).

| Compound | | |
|---|---|---|
| Number | Synthesis | LC/MS |
| 753 | Example 133 | m/z 633.68 [M + H]+ |

### Example 134

### 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylic acid (Compound 754)

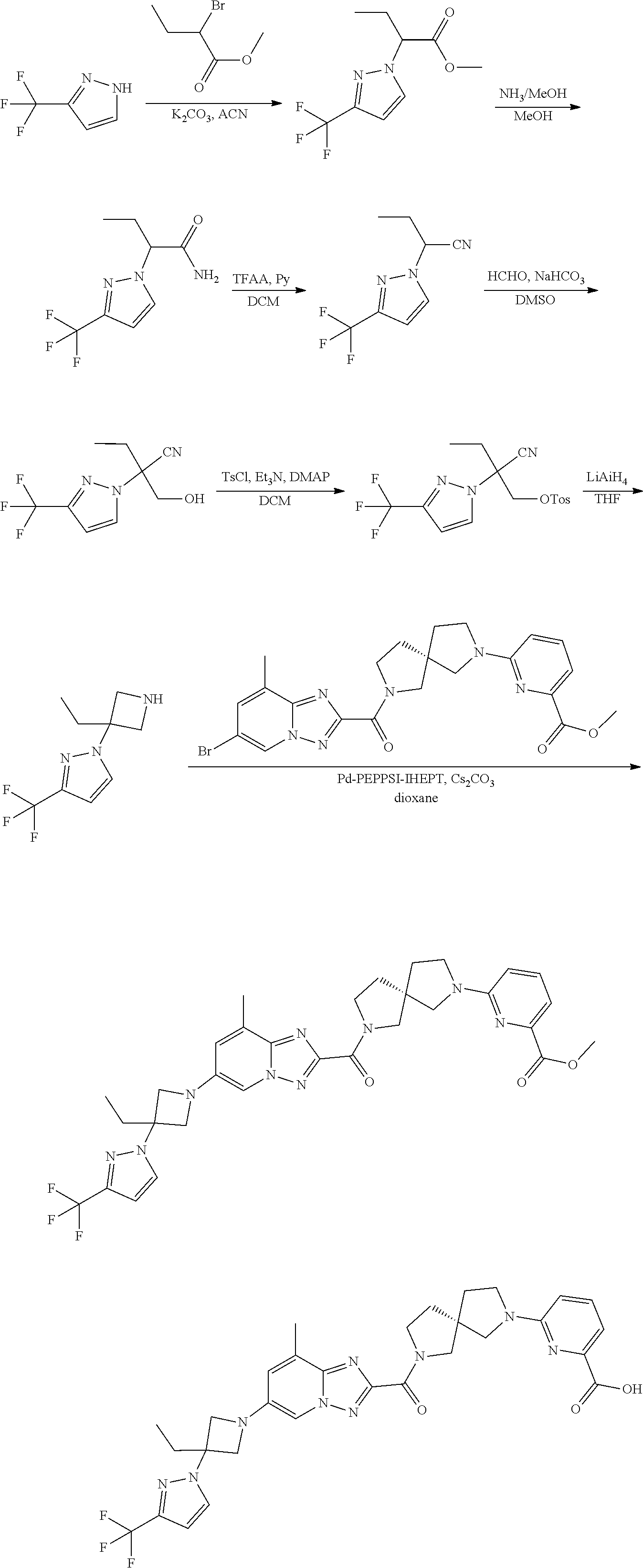

### Step 1: methyl 2-[3-(trifluoromethyl)pyrazol-1-yl]butanoate

To a solution of 3-(trifluoromethyl)-1H-pyrazole (5 g, 36.74 mmol, 1 eq) in ACN (50 mL) was added K₂CO₃ (12.70 g, 91.86 mmol, 2.5 eq) and methyl 2-bromobutanoate (7.98 g, 44.09 mmol, 5.07 mL, 1.2 eq) under N₂ atmosphere. The mixture was stirred at 25° C. for 1 h under N₂ atmosphere. LCMS showed starting material was consumed. Several new peaks were shown on LCMS and 88% of desired compound was detected. The reaction mixture was filtered and concentrated under reduced pressure to give a residue. The residue was purified by flash silica gel chromatography (SiO₂, PE:EtOAc=3:1, Rf=0.5) to give compound methyl 2-[3-(trifluoromethyl)pyrazol-1-yl]butanoate (7.58 g, 85.77% yield) as white oil.

LC-MS [ESI, M+1]: 237.1.

¹H NMR (400 MHz, CHLOROFORM-d) δ=7.64 (d, J=1.2 Hz, 1H), 6.60 (d, J=2.0 Hz, 1H), 4.96 (dd, J=6.0, 9.6 Hz, 1H), 3.77 (s, 3H), 2.34-2.09 (m, 2H), 0.92 (t, J=7.6 Hz, 3H).

### Step 2: 2-[3-(trifluoromethyl)pyrazol-1-yl]butanamide

A mixture of methyl 2-[3-(trifluoromethyl)pyrazol-1-yl]butanoate (6.58 g, 27.86 mmol, 1 eq) in NH₃/MeOH (130 mL) stirred at 60° C. for 12 h. LCMS showed starting material was consumed. Several new peaks were shown on LCMS and desired compound was detected. The reaction mixture was concentrated under reduced pressure to give a crude 2-[3-(trifluoromethyl)pyrazol-1-yl]butanamide (6.1 g, 95.33% yield) as yellow solid.

LC-MS [ESI, M+1]: 222.1.

### Step 3: 2-[3-(trifluoromethyl)pyrazol-1-yl]butanenitrile

To a solution of 2-[3-(trifluoromethyl)pyrazol-1-yl]butanamide (5.62 g, 25.41 mmol, 1 eq) in DCM (110 mL) was added Pyridine (10.05 g, 127.05 mmol, 10.25 mL, 5 eq) and TFAA (10.67 g, 50.82 mmol, 7.06 mL, 2 eq) at 0° C. The mixture was stirred at 25° C. for 1 h under N₂ atmosphere. TLC indicated starting material was consumed and one new spot formed. (PE:EtOAc=3:1, Rf=0.5). The reaction mixture was diluted with H₂O (15 mL) and extracted with DCM (30 mL×3). The combined organic layers were washed with brine (5 mL×3), dried over Na₂SO₄, filtered and concentrated under reduced pressure to give a residue. The residue was purified by flash silica gel chromatography (SiO₂, PE:EtOAc=3:1, Rf=0.5) to give compound 2-[3-(trifluoromethyl)pyrazol-1-yl]butanenitrile (5 g, 96.86% yield) as yellow solid.

¹H NMR (400 MHz, CHLOROFORM-d) δ=7.68 (d, J=1.6 Hz, 1H), 6.64 (d, J=2.4 Hz, 1H), 5.21 (t, J=7.2 Hz, 1H), 2.38-2.20 (m, 2H), 1.08 (t, J=7.6 Hz, 3H)

### Step 4: 2-(hydroxymethyl)-2-[3-(trifluoromethyl)pyrazol-1-yl]butanenitrile

To a solution of 2-[3-(trifluoromethyl)pyrazol-1-yl]butanenitrile (2 g, 9.84 mmol, 1 eq) in DMSO (20 mL) was added NaHCO₃ (82.70 mg, 984.43 μmol, 38.30 μL, 0.1 eq). Then Paraformaldehyde (1.06 g, 35.44 mmol, 3.6 eq) was added. The resulting mixture was stirred at 25° C. for 12 h. TLC indicated starting material was consumed and one new spot formed. (PE:EtOAc=2:1, Rf=0.4). The reaction mixture was diluted with H₂O (20 mL) and extracted with EtOAC (30 mL×3). The combined organic layers were washed with brine (10 mL×3), dried over Na₂SO₄, filtered and concentrated under reduced pressure to give a residue. The residue was purified by flash silica gel chromatography (SiO₂, PE:EtOAc=2:1, Rf=0.4) to give compound 2-(hydroxymethyl)-2-[3-(trifluoromethyl)pyrazol-1-yl]butanenitrile (1.71 g, 74.49% yield) as white oil.

¹H NMR (400 MHz, CHLOROFORM-d) δ=7.96-7.83 (m, 1H), 6.64 (d, J=2.4 Hz, 1H), 4.31-4.14 (m, 2H), 2.85-2.71 (m, 1H), 2.40-2.27 (m, 1H), 2.26-2.14 (m, 1H), 1.03 (t, J=7.6 Hz, 3H)

### Step 5: [2-cyano-2-[3-(trifluoromethyl)pyrazol-1-yl]butyl]4-methylbenzenesulfonate

To a solution of 2-(hydroxymethyl)-2-[3-(trifluoromethyl)pyrazol-1-yl]butanenitrile (1.71 g, 7.33 mmol, 1 eq) in DCM (17 mL) was added 4-methylbenzenesulfonyl chloride (2.80 g, 14.67 mmol, 2 eq), Et₃N (2.23 g, 22.00 mmol, 3.06 mL, 3 eq) and DMAP (179.17 mg, 1.47 mmol, 0.2 eq). The mixture was stirred at 25° C. for 12 h. TLC indicated starting material was consumed and one new spot formed. (PE:EtOAc=3:1, Rf=0.5). The reaction mixture was diluted with H₂O (10 mL) and extracted with DCM (20 mL×3). The combined organic layers were washed with brine (5 mL×3), dried over Na₂SO₄, filtered and concentrated under reduced pressure to give a residue. The residue was purified by flash silica gel chromatography (SiO₂, PE:EtOAc=3:1, Rf=0.5) to give compound [2-cyano-2-[3-(trifluoromethyl)pyrazol-1-yl]butyl]4-methylbenzenesulfonate (2.6 g, 91.53% yield) as white oil.

¹H NMR (400 MHz, CHLOROFORM-d) δ=7.82 (d, J=1.6 Hz, 1H), 7.64 (d, J=8.3 Hz, 2H), 7.32 (d, J=8.4 Hz, 2H), 6.54 (d, J=2.4 Hz, 1H), 4.44 (s, 2H), 2.45 (s, 3H), 2.43-2.34 (m, 1H), 2.17 (qd, J=7.2, 14.3 Hz, 1H), 0.96 (t, J=7.2 Hz, 3H)

### Step 6: 1-(3-ethylazetidin-3-yl)-3-(trifluoromethyl)pyrazole

To a solution of LiAlH₄ (2.5 M, 1.03 mL, 5 eq) in THF (0.5 mL) was added [2-cyano-2-[3-(trifluoromethyl)pyrazol-1-yl]butyl]4-methylbenzenesulfonate (200 mg, 516.29 μmol, 1 eq) in THF (1 mL) dropwise at 0° C. under N₂. The mixture was stirred at 25° C. for 1 h under N₂. LCMS showed starting material was consumed. Several new peaks were shown on LCMS and desired compound was detected. The reaction mixture was quenched with H₂O (0.2 mL), 15% NaOH (0.2 mL) and H₂O (0.6 mL) at 0° C., then was diluted with H₂O (5 mL) and extracted with EtOAc (10 mL×3). The combined organic layers were washed with brine (5 mL×3), dried over Na₂SO₄, filtered and concentrated under reduced pressure to give a residue. The residue was purified by prep-TLC (SiO₂, DCM:MeOH=10:1, Rf=0.5) and eluted with DCM and MeOH (10:1, 50 mL) to give the product 1-(3-ethylazetidin-3-yl)-3-(trifluoromethyl)pyrazole (63 mg, 54.78% yield) as white oil.

LC-MS [ESI, M+1]: 220.2.

¹H NMR (400 MHz, CHLOROFORM-d) δ=7.48 (d, J=1.2 Hz, 1H), 6.56 (d, J=2.4 Hz, 1H), 4.73 (br s, 1H), 4.16 (d, J=9.2 Hz, 2H), 3.75 (d, J=9.2 Hz, 2H), 2.24 (q, J=7.6 Hz, 2H), 0.76 (t, J=7.2 Hz, 3H)

### Step 7: methyl 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylate

To a solution of 1-(3-ethylazetidin-3-yl)-3-(trifluoromethyl)pyrazole (60 mg, 273.71 μmol, 1 eq) in dioxane (2 mL) was added methyl 6-[(5R)-2-(6-bromo-8-methyl-[1,2,4]triazolo[1,5-a]pyridine-2-carbonyl)-2,7-diazaspiro[4.4]nonan-7-yl]pyridine-2-carboxylate (136.68 mg, 273.71 μmol, 1 eq), dicesium;carbonate (222.95 mg, 684.29 μmol, 2.5 eq) and 1,3-bis[2,6-bis(1-propylbutyl)phenyl]-4,5-dichloro-2H-imidazol-1-ium-2-ide;3-chloropyridine;dichloropalladium (13.31 mg, 13.69 μmol, 0.05 eq) under N₂. The mixture was stirred at 100° C. for 1 h under N₂. LCMS showed starting material was consumed. Several new peaks were shown on LCMS and desired compound was detected. The reaction mixture was filtered and concentrated under reduced pressure to give a residue. The crude product was purified by reversed-phase HPLC (0.1% FA condition) to give compound methyl 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylate (68 mg, 37.75% yield) as white solid.

LC-MS [ESI, M+1]: 638.1.

¹H NMR (400 MHz, CHLOROFORM-d) δ=7.70-7.50 (m, 3H), 7.38 (t, J=7.2 Hz, 1H), 6.80 (br d, J=8.0 Hz, 1H), 6.67-6.45 (m, 2H), 4.36 (t, J=7.7 Hz, 2H), 4.15 (t, J=7.6 Hz, 3H), 4.02-3.86 (m, 5H), 3.79 (q, J=12.4 Hz, 1H), 3.72-3.44 (m, 4H), 2.62 (d, J=10.4 Hz, 3H), 2.37-2.25 (m, 2H), 2.20-1.95 (m, 4H), 0.93-0.79 (m, 3H)

### Step 8: 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylic acid

To a solution of methyl 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylate (60 mg, 94.09 μmol, 1 eq) in MeOH (0.4 mL) and H₂O (0.1 mL) was added NaOH (7.53 mg, 188.19 μmol, 2 eq). The mixture was stirred at 25° C. for 0.5 h. LCMS showed starting material was consumed. One main peak was shown on LCMS and desired compound was detected. The reaction was adjusted pH to 7 with FA (0.1 mL) and then was concentrated under reduced pressure to give a residue. The residue was purified by prep-HPLC (FA condition; column: CD01-Phenomenex luna C18 150×25×10 um; mobile phase: [H₂O (0.225% FA)—ACN]; gradient: 30%-60% B over 10.0 min) to give compound 6-[(5R)-2-[6-[3-ethyl-3-[3-(trifluoromethyl)pyrazol-1-yl]azetidin-1-yl]-8-methyl-[1,2,4]triazolo[1,5-a]pyridine-2-carbonyl]-2,7-diazaspiro[4.4]nonan-7-yl]pyridine-2-carboxylic acid (39.04 mg, 66.33% yield) as white solid.

LC-MS [ESI, M+1]: 624.3.

¹H NMR (400 MHz, CHLOROFORM-d) δ=7.73-7.53 (m, 3H), 7.48 (t, J=6.4 Hz, 1H), 6.81 (br d, J=9.6 Hz, 1H), 6.69-6.55 (m, 2H), 4.46-4.28 (m, 2H), 4.26-4.08 (m, 3H), 3.99 (s, 1H), 3.91 (t, J=7.2 Hz, 1H), 3.86-3.73 (m, 1H), 3.70-3.44 (m, 4H), 2.62 (d, J=11.6 Hz, 3H), 2.30 (t, J=7.2 Hz, 2H), 2.20-2.03 (m, 4H), 0.94-0.79 (m, 3H)

| Compound | | |
|---|---|---|
| Number | Synthesis | LC/MS |
| 754 | Example 134 | m/z 632.64 [M + H]+ |

### Example 135

### 6-((R)-7-(6-((S)-3-(3-fluorophenyl)-3-methylpyrrolidin-1-yl)-8-methyl-[1,2,4]triazolo[1,5-a]pyridine-2-carbonyl)-2,7-diazaspiro[4.4]nonan-2-yl)picolinic acid (Compound 763)

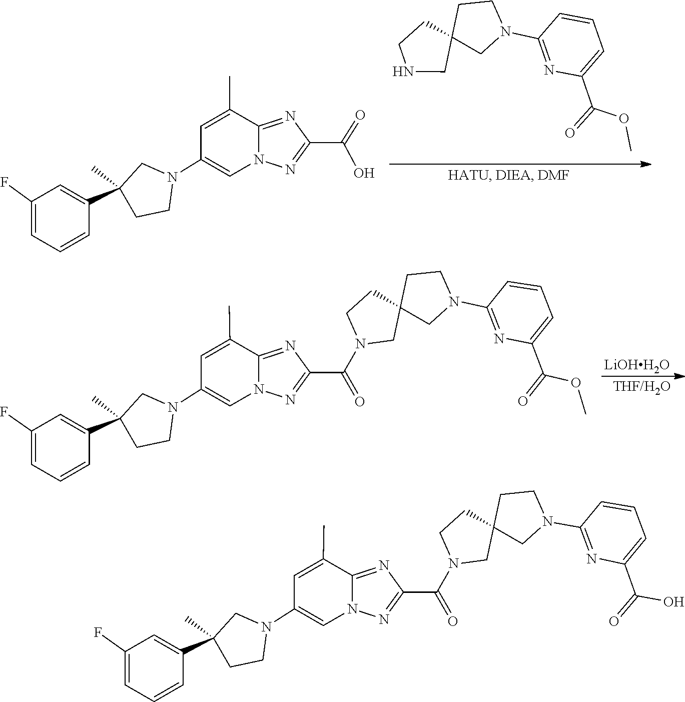

### Step 1: methyl 6-((R)-7-(6-((S)-3-(3-fluorophenyl)-3-methylpyrrolidin-1-yl)-8-methyl-[1,2,4]triazolo[1,5-a]pyridine-2-carbonyl)-2,7-diazaspiro[4.4]nonan-2-yl)picolinate

To a solution of 6-[(3S)-3-(3-fluorophenyl)-3-methyl-pyrrolidin-1-yl]-8-methyl-[1,2,4]triazolo[1,5-a]pyridine-2-carboxylic acid (30 mg, 84.66 μmol, 1 eq) and methyl 6-[(5R)-2,7-diazaspiro[4.4]nonan-2-yl]pyridine-2-carboxylate (22.12 mg, 84.66 μmol, 1 eq) in DMF (3 mL) was added HATU (64.38 mg, 169.31 μmol, 2 eq) and DIEA (32.82 mg, 253.97 μmol, 44.24 μL, 3 eq). The mixture was stirred at 25° C. for 1 hr. The reaction mixture was added H₂O (10 ml) and then was extracted with EtOAc (10 mL×3), The combined organic layers were washed with brine (30 mL×3), dried over anhydrous Na₂SO₄, filtered and dried to give compound methyl 6-((R)-7-(6-((S)-3-(3-fluorophenyl)-3-methylpyrrolidin-1-yl)-8-methyl-[1,2,4]triazolo[1,5-a]pyridine-2-carbonyl)-2,7-diazaspiro[4.4]nonan-2-yl)picolinate (50 mg, crude) as a yellow solid.

LC-MS [ESI, M+1]: 598.2

### Step 2: 6-((R)-7-(6-((S)-3-(3-fluorophenyl)-3-methylpyrrolidin-1-yl)-8-methyl-[1,2,4]triazolo[1,5-a]pyridine-2-carbonyl)-2,7-diazaspiro[4.4]nonan-2-yl)picolinic acid

To a solution of methyl 6-[(5R)-2-[6-[(3S)-3-(3-fluorophenyl)-3-methyl-pyrrolidin-1-yl]-8-methyl-[1,2,4]triazolo[1,5-a]pyridine-2-carbonyl]-2,7-diazaspiro[4.4]nonan-7-yl]pyridine-2-carboxylate (50 mg, 83.66 μmol, 1 eq) in THF (1 mL) and H₂O (0.2 mL) was added LiOH·H2O (10.53 mg, 250.97 μmol, 3 eq). The mixture was stirred at 25° C. for 15 min. The reaction mixture was concentrated under the reduced pressure to give the product. The residue was purified by prep-HPLC (column: CD01-Phenomenex luna C18 150*25*10 um; mobile phase: [water(FA)—ACN]; gradient:30%-60% B over 8 min) and lyophilized to give compound 6-((R)-7-(6-((S)-3-(3-fluorophenyl)-3-methylpyrrolidin-1-yl)-8-methyl-[1,2,4]triazolo[1,5-a]pyridine-2-carbonyl)-2,7-diazaspiro[4.4]nonan-2-yl)picolinic acid (16.73 mg, 26.54 μmol, 31.73% yield, 99.902% purity, FA) as a yellow solid.

LC-MS [ESI, M+1]: 584.2

¹H NMR (CHLOROFORM-d, 400 MHz): δ=8.25 (s, 0.3H), 7.57-7.74 (m, 2H), 7.47 (t, J=6.4 Hz, 1H), 7.28-7.39 (m, 1H), 7.08 (t, J=7.6 Hz, 1H), 6.90-7.04 (m, 3H), 6.62 (t, J=8.0 Hz, 1H), 4.09-4.31 (m, 1H), 4.02 (s, 1H), 3.91 (t, J=7.2 Hz, 1H), 3.71-3.87 (m, 1H), 3.56-3.70 (m, 3H), 3.44-3.56 (m, 4.1H), 3.42 (dd, J=8.8, 3.8 Hz, 1H), 2.63 (d, J=10.8 Hz, 3H), 2.34 (d, J=7.2 Hz, 1H), 2.25 (s, 1H), 1.94-2.18 (m, 4.0H), 1.46 ppm (d, J=8.0 Hz, 3.0H).

| Compound | | |
|---|---|---|
| Number | Synthesis | LC/MS |
| 755 | Similar to Example 135 | m/z 584.5 [M + H]⁺ |

### Example 136

### 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylic acid (Compound 756)

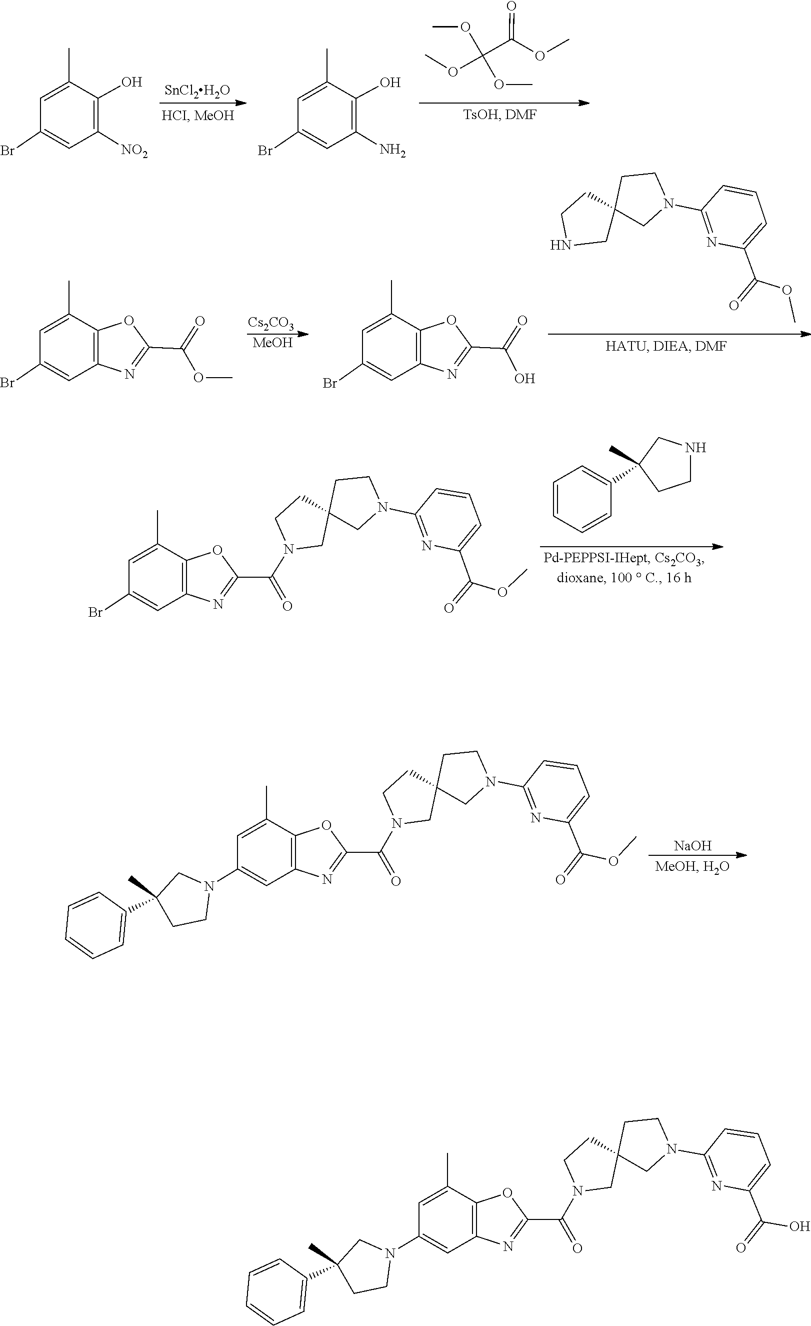

### Step 1:2-amino-4-bromo-6-methyl-phenol

To a solution of 4-bromo-2-methyl-6-nitro-phenol (1.6 g, 6.90 mmol, 1 eq) in MeOH (20 mL) was added HCl (12 M, 5.75 mL, 10 eq) and SnCl₂·H₂O (7.78 g, 34.48 mmol, 5 eq). The mixture was stirred at 25° C. for 12 h. LCMS showed starting material was consumed and the desired mass was detected. The reaction mixture was diluted with water (10 mL) and basified with aqueous NaHCO₃ till pH around 8, the mixture was extracted with DCM (20 mL×3), the combined organic layers were dried over anhydrous sodium sulfate, filtered and the filtrate was concentrated under reduced pressure to give compound 2-amino-4-bromo-6-methyl-phenol (830 mg, 59.57% yield) as a white solid.

LC-MS [ESI, M+1]: 204.1.

¹H NMR (400 MHz, DMSO-d₆) δ=6.61 (d, J=2.4 Hz, 1H), 6.44 (d, J=2.4 Hz, 1H), 4.84 (br s, 2H), 2.08 (s, 3H).

### Step 2: methyl 5-bromo-7-methyl-1,3-benzoxazole-2-carboxylate

To a solution of 2-amino-4-bromo-6-methyl-phenol (820 mg, 4.06 mmol, 1 eq) in methyl 2,2,2-trimethoxyacetate (6.66 g, 40.58 mmol, 10 eq) was added TsOH (34.94 mg, 202.92 μmol, 0.05 eq). The mixture was stirred at 80° C. for 1 h. LCMS showed starting material was consumed and the desired mass was detected. The reaction was filtered and the filtered concentrated under reduced pressure to give a residue. The crude product was purified by reversed-phase HPLC (0.1% FA condition) to give compound methyl 5-bromo-7-methyl-1,3-benzoxazole-2-carboxylate (360 mg, 32.84% yield) as a white solid.

LC-MS [ESI, M+1]:270.0

### Step 3: 5-bromo-7-methyl-1,3-benzoxazole-2-carboxylic acid

To a solution of methyl 5-bromo-7-methyl-1,3-benzoxazole-2-carboxylate (50 mg, 185.13 μmol, 1 eq) in MeOH (1 mL) was added NaOH (1 M, 925.65 μL, 5 eq). The mixture was stirred at 25° C. for 1 h. LCMS showed starting material was consumed and the desired mass was detected. The reaction was filtered and the filtered concentrated under reduced pressure to give a residue. The residue was purified by flash silica gel chromatography (SiO₂, PE:EtOAc=5:1, Rf=0.3) to give compound 5-bromo-7-methyl-1,3-benzoxazole-2-carboxylic acid (40 mg, 84.38% yield) as a white solid.

LC-MS [ESI, M+1]:258.0

¹H NMR (400 MHz, DMSO-d₆) δ=7.73 (d, J=1.2 Hz, 1H), 7.41 (d, J=0.8 Hz, 1H), 2.48 (s, 3H).

### Step 4: methyl 6-[(5R)-2-(5-bromo-7-methyl-1,3-benzoxazole-2-carbonyl)-2,7-diazaspiro[4.4]nonan-7-yl]pyridine-2-carboxylate

To a solution of 5-bromo-7-methyl-1,3-benzoxazole-2-carboxylic acid (30 mg, 117.16 μmol, 1 eq) and methyl 6-[(5R)-2,7-diazaspiro[4.4]nonan-2-yl]pyridine-2-carboxylate (30.62 mg, 117.16 μmol, 1 eq) in DMF (2 mL) was added HATU (66.82 mg, 175.75 μmol, 1.5 eq) and DIEA (45.43 mg, 351.49 μmol, 61.22 μL, 3 eq). The mixture was stirred at 25° C. for 1 h. LCMS showed starting material was consumed and the desired mass was detected. The reaction was filtered and the filtered concentrated under reduced pressure to give a residue. The crude product was purified by reversed-phase HPLC (0.1% FA condition) to give compound methyl 6-[(5R)-2-(5-bromo-7-methyl-1,3-benzoxazole-2-carbonyl)-2,7-diazaspiro[4.4]nonan-7-yl]pyridine-2-carboxylate (26 mg, 44.44% yield) as a white solid.

LC-MS [ESI, M+1]: 499.0

### Step 5: methyl 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylate

A mixture of methyl 6-[(5R)-2-(5-bromo-7-methyl-1,3-benzoxazole-2-carbonyl)-2,7-diazaspiro[4.4]nonan-7-yl]pyridine-2-carboxylate (25 mg, 50.06 μmol, 1 eq), (3R)-3-methyl-3-phenyl-pyrrolidine (12.11 mg, 75.10 μmol, 1.5 eq), Cs₂CO₃ (48.94 mg, 150.19 μmol, 3 eq) and 1,3-bis[2,6-bis(1-propylbutyl)phenyl]-4,5-dichloro-2H-imidazol-1-ium-2-ide;3-chloropyridine;dichloropalladium (4.87 mg, 5.01 μmol, 0.1 eq) in 1,4-dioxane (1 mL) was degassed and purged with N₂ for 3 times, and then the mixture was stirred at 100° C. for 1 h under N₂ atmosphere. LCMS showed starting material was consumed and the desired mass was detected. The reaction was filtered and the filtered concentrated under reduced pressure to give a residue. The crude product was purified by reversed-phase HPLC (0.1% FA condition) to give compound methyl 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylate (24 mg, 82.70% yield) as a yellow solid.

LC-MS [ESI, M+1]: 580.3

### Step 6: 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylic acid

To a solution of methyl 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylate (20 mg, 34.50 μmol, 1 eq) in MeOH (1 mL) was added Cs₂CO₃ (33.72 mg, 103.50 μmol, 3 eq) The mixture was stirred at 25° C. for 1 h. LCMS showed starting material was consumed and the desired mass was detected. The reaction was filtered and the filtered concentrated under reduced pressure to give a residue. The residue was purified by prep-HPLC (column: CD24-WePure Biotech XPT C18 150*25*7 um; mobile phase: [H₂O (10 mM NH₄HCO₃)—ACN (0.02% CH₃COOH)]; gradient:36%-66% B over 8.0 min). The result solution was lyophilized to give compound 6-[(5R)-2-[7-methyl-5-[(3R)-3-methyl-3-phenyl-pyrrolidin-1-yl]-1,3-benzoxazole-2-carbonyl]-2,7-diazaspiro[4.4]nonan-7-yl]pyridine-2-carboxylic acid (4.29 mg, 21.58% yield, 98.188% purity) as a yellow solid.

LC-MS [ESI, M+1]: 566.2

¹H NMR (400 MHz, CHLOROFORM-d) δ=7.67 (dt, J=3.2, 7.8 Hz, 1H), 7.57-7.45 (m, 1H), 7.40-7.30 (m, 4H), 7.27-7.18 (m, 1H), 6.73-6.57 (m, 3H), 4.42-4.25 (m, 1H), 4.18 (s, 1H), 3.92 (br t, J=6.4 Hz, 1H), 3.80 (q, J=12.8 Hz, 1H), 3.71-3.49 (m, 7H), 3.47-3.38 (m, 1H), 2.56 (d, J=4.8 Hz, 3H), 2.38-2.23 (m, 2H), 2.19-2.03 (m, 4H), 1.45 (d, J=9.2 Hz, 3H).

| Compound | | |
|---|---|---|
| Number | Synthesis | LC/MS |
| 756 | Example 136 | m/z 566.2[M + H]⁺ |

### Example 138

### (1R,4r)-4-(8-(2-amino-2-oxoethyl)-6-((R)-3-methyl-3-phenylpyrrolidin-1-yl)-[1,2,4]triazolo[1,5-a]pyridine-2-carboxamido)cyclohexane-1-carboxylic acid (Compound 758)

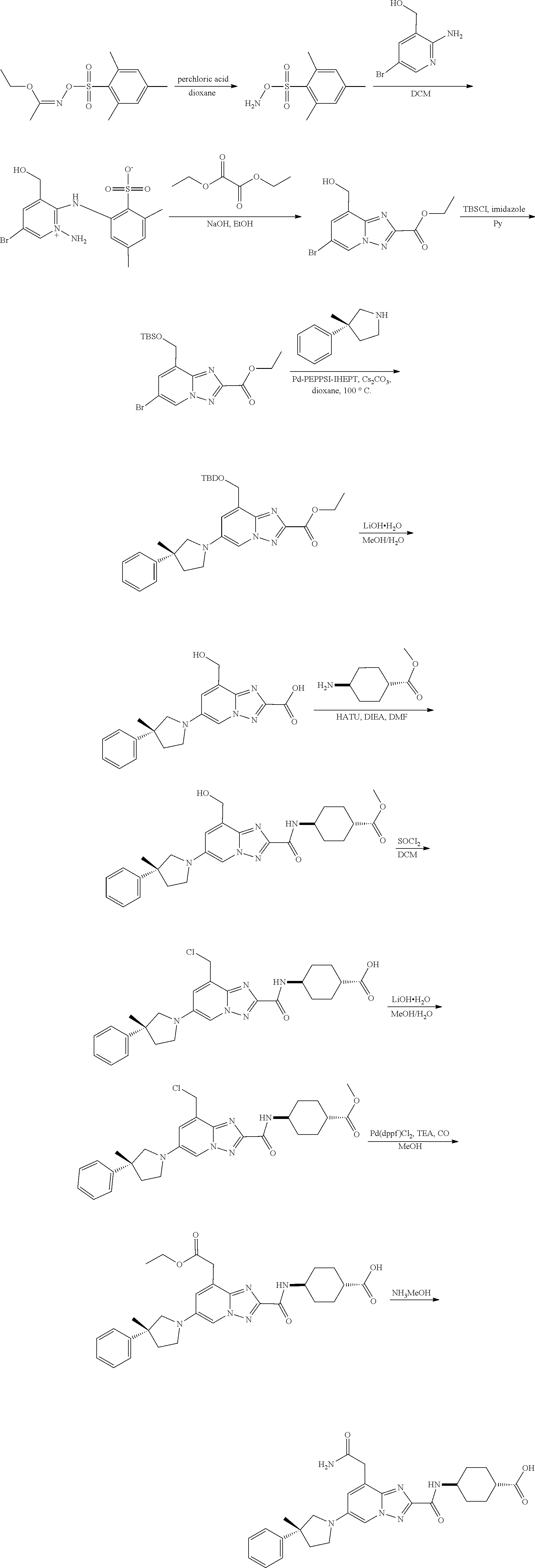

### Step 1: amino 2,4,6-trimethylbenzenesulfonate

To a solution of ethyl (1E)-N-(2,4,6-trimethylphenyl)sulfonyloxyethanimidate (50 g, 175.22 mmol, 1 eq) in dioxane (500 mL) was added perchloric acid (100.59 g, 700.87 mmol, 60.59 mL, 70% purity, 4 eq) dropwise at −5° C. The resulting mixture was stirred at 0° C. for 0.5 h. TLC indicated starting material was consumed completely and one new spot formed (PE:EtOAc=3:1, Rf=0.4). After reaction completed, ice-water (800 mL) was added in portions, the resulting precipitate was collected by filtration and washed with water to give the crude amino 2,4,6-trimethylbenzenesulfonate (37.2 g, 98.62% yield) as brown solid.

### Step 2: (1,2-diamino-5-bromo-pyridin-1-ium-3-yl)methanol;2,4,6-trimethylbenzenesulfonate

To a solution of (2-amino-5-bromo-3-pyridyl)methanol (35 g, 172.38 mmol, 1 eq) in DCM (500 mL) was added amino 2,4,6-trimethylbenzenesulfonate (37.11 g, 172.38 mmol, 1 eq) in DCM (200 mL) dropwise at 0° C. The mixture was stirred at 0° C. for 0.5 h. LCMS showed starting material was consumed. Several new peaks were shown on LCMS and 40% of desired compound was detected. The reaction mixture was filtered and concentrated under reduced pressure to give a crude (1,2-diamino-5-bromo-pyridin-1-ium-3-yl)methanol;2,4,6-trimethylbenzenesulfonate (65.2 g, 90.42% yield) as brown solid.

LC-MS [ESI, M-202+1]: 218.0.

¹H NMR (400 MHz, DMSO-d₆) δ=8.41-8.21 (m, 3H), 7.89 (s, 1H), 6.83 (s, 2H), 6.74 (s, 2H), 5.76 (s, 1H), 4.43 (s, 2H), 2.49 (s, 6H), 2.16 (s, 3H)

### Step 3: ethyl 6-bromo-8-(hydroxymethyl)-[1,2,4]triazolo[1,5-a]pyridine-2-carboxylate

To a solution of (1,2-diamino-5-bromo-pyridin-1-ium-3-yl)methanol;2,4,6-trimethylbenzenesulfonate (30 g, 71.72 mmol, 1 eq) and diethyl oxalate (20.96 g, 143.44 mmol, 19.59 mL, 2 eq) in EtOH (600 mL) was added NaOH (4.30 g, 107.58 mmol, 1.5 eq). The mixture was stirred at 70° C. for 0.5 h. LCMS showed starting material was consumed. Several new peaks were shown on LCMS and desired compound was detected. The reaction mixture was concentrated under reduced pressure to give a residue. The residue was purified by column chromatography (SiO₂, PE:EtOAc=1:1, Rf=0.5) to give compound ethyl 6-bromo-8-(hydroxymethyl)-[1,2,4]triazolo[1,5-a]pyridine-2-carboxylate (12.2 g, 50.56% yield) as brown solid.

LC-MS [ESI, M+1]: 300.0.

¹H NMR (400 MHz, DMSO-d₆) δ=9.38 (d, J=0.8 Hz, 1H), 7.77 (d, J=1.2 Hz, 1H), 6.03-5.58 (m, 1H), 4.87 (s, 2H), 4.47-4.36 (m, 2H), 1.35 (t, J=7.2 Hz, 3H)

### Step 4: ethyl 6-bromo-8-[[tert-butyl(dimethyl)silyl]oxymethyl]-[1,2,4]triazolo[1,5-a]pyridine-2-carboxylate

To a solution of ethyl 6-bromo-8-(hydroxymethyl)-[1,2,4]triazolo[1,5-a]pyridine-2-carboxylate (12.2 g, 40.65 mmol, 1 eq) in Py (120 mL) was added imidazole (6.92 g, 101.63 mmol, 2.5 eq) and TBSCl (15.32 g, 101.63 mmol, 12.50 mL, 2.5 eq) at 0° C. slowly. The mixture was stirred at 25° C. for 1 h. LCMS showed starting material was consumed. Several new peaks were shown on LCMS and desired compound was detected. The reaction mixture was concentrated under reduced pressure to give a residue. The residue was purified by flash silica gel chromatography (SiO₂, PE:EtOAc=2:1, Rf=0.4) to give compound ethyl 6-bromo-8-[[tert-butyl(dimethyl)silyl]oxymethyl]-[1,2,4]triazolo[1,5-a]pyridine-2-carboxylate (4.8 g, 53.33% yield) as brown solid.

LC-MS [ESI, M+1]: 415.8.

¹H NMR (400 MHz, CHLOROFORM-d) δ=8.66 (s, 1H), 7.79 (d, J=1.6 Hz, 1H), 5.18 (s, 2H), 4.56 (q, J=7.2 Hz, 2H), 1.47 (t, J=7.2 Hz, 3H), 0.98 (s, 9H), 0.16 (s, 6H)

### Step 5: ethyl 8-[[tert-butyl(dimethyl)silyl]oxymethyl]-6-[(3R)-3-methyl-3-phenyl-pyrrolidin-1-yl]-[1,2,4]triazolo[1,5-a]pyridine-2-carboxylate

To a solution of ethyl 6-bromo-8-[[tert-butyl(dimethyl)silyl]oxymethyl]-[1,2,4]triazolo[1,5-a]pyridine-2-carboxylate (4 g, 9.65 mmol, 1 eq) and (3R)-3-methyl-3-phenyl-pyrrolidine (1.56 g, 9.65 mmol, 1 eq) in dioxane (50 mL) was added 1,3-bis[2,6-bis(1-propylbutyl)phenyl]-4,5-dichloro-2H-imidazol-1-ium-2-ide;3-chloropyridine;dichloropalladium (469.52 mg, 482.66 μmol, 0.05 eq) and dicesium;carbonate (7.86 g, 24.13 mmol, 2.5 eq). The mixture was stirred at 100° C. for 1 h under N₂. LCMS showed starting material consumed. Several new peaks were shown on LCMS and desired compound was detected. The reaction mixture was filtered and concentrated under reduced pressure to give a residue. The crude product was purified by reversed-phase HPLC (0.1% FA condition) to give compound ethyl 8-[[tert-butyl(dimethyl)silyl]oxymethyl]-6-[(3R)-3-methyl-3-phenyl-pyrrolidin-1-yl]-[1,2,4]triazolo[1,5-a]pyridine-2-carboxylate (2.4 g, 49.70% yield) as yellow solid.

LC-MS [ESI, M+1]: 495.6.

¹H NMR (400 MHz, CHLOROFORM-d) δ=7.70 (d, J=1.6 Hz, 1H), 7.44 (d, J=1.6 Hz, 1H), 7.42-7.35 (m, 2H), 7.34-7.27 (m, 3H), 5.19 (s, 2H), 4.55 (q, J=7.2 Hz, 2H), 3.63 (d, J=9.2 Hz, 1H), 3.57 (q, J=8.4 Hz, 1H), 3.52-3.42 (m, 2H), 2.40 (td, J=8.4, 12.0 Hz, 1H), 2.33-2.23 (m, 1H), 1.52-1.46 (m, 6H), 1.01 (s, 9H), 0.17 (d, J=1.6 Hz, 6H)

### Step 6: 8-(hydroxymethyl)-6-[(3R)-3-methyl-3-phenyl-pyrrolidin-1-yl]-[1,2,4]triazolo[1,5-a]pyridine-2-carboxylic acid

To a solution of ethyl 8-[[tert-butyl(dimethyl)silyl]oxymethyl]-6-[(3R)-3-methyl-3-phenyl-pyrrolidin-1-yl]-[1,2,4]triazolo[1,5-a]pyridine-2-carboxylate (200 mg, 404.29 μmol, 1 eq) in H₂O (0.5 mL) and MeOH (2 mL) was added LiOH·H₂O (33.93 mg, 808.57 μmol, 2 eq). The mixture was stirred at 25° C. for 0.5 h. LCMS showed starting material consumed. One main peak was shown on LCMS and desired compound was detected. The reaction was adjusted pH to 7 with FA (0.1 mL) and then was concentrated under reduced pressure to give a residue. The crude product was purified by reversed-phase HPLC (0.1% FA condition) to give compound 8-(hydroxymethyl)-6-[(3R)-3-methyl-3-phenyl-pyrrolidin-1-yl]-[1,2,4]triazolo[1,5-a]pyridine-2-carboxylic acid (124 mg, 87.04% yield) as yellow solid.

LC-MS [ESI, M+1]: 353.1.

¹H NMR (400 MHz, DMSO-d₆) δ=7.98 (s, 1H), 7.47-7.30 (m, 5H), 7.29-7.19 (m, 1H), 5.77-5.42 (m, 1H), 4.84 (s, 2H), 3.66-3.59 (m, 1H), 3.58-3.48 (m, 2H), 3.44-3.38 (m, 1H), 2.36-2.21 (m, 2H), 1.37 (s, 3H)

### Step 7: methyl (1R,4r)-4-(8-(hydroxymethyl)-6-((R)-3-methyl-3-phenylpyrrolidin-1-yl)-[1,2,4]triazolo[1,5-a]pyridine-2-carboxamido)cyclohexane-1-carboxylate

To a solution of 8-(hydroxymethyl)-6-[(3R)-3-methyl-3-phenyl-pyrrolidin-1-yl]-[1,2,4]triazolo[1,5-a]pyridine-2-carboxylic acid (124 mg, 351.89 μmol, 1 eq) and methyl (1r, 4r)-4-aminocyclohexane-1-carboxylate (82.98 mg, 527.83 μmol, 1.5 eq) in DMF (1.5 mL) was added HATU (200.70 mg, 527.83 μmol, 1.5 eq) and DIEA (181.91 mg, 1.41 mmol, 245.17 μL, 4 eq). The mixture was stirred at 25° C. for 0.5 h. LCMS showed starting material consumed. Several peaks were shown on LCMS and desired compound was detected. The reaction mixture was filtered and concentrated under reduced pressure to give compound methyl (1R,4r)-4-(8-(hydroxymethyl)-6-((R)-3-methyl-3-phenylpyrrolidin-1-yl)-[1,2,4]triazolo[1,5-a]pyridine-2-carboxamido)cyclohexane-1-carboxylate (87 mg, 49.79% yield) as white solid.

LC-MS [ESI, M+1]: 492.3.

¹H NMR (400 MHz, METHANOL-d4) δ=7.88 (br s, 1H), 7.49 (br s, 1H), 7.43-7.29 (m, 4H), 7.22 (br s, 1H), 4.97 (br s, 2H), 3.98-3.85 (m, 1H), 3.68 (br s, 3H), 3.62 (br s, 2H), 3.48 (br s, 2H), 2.44-2.29 (m, 3H), 2.08 (br d, J=10.4 Hz, 4H), 1.67-1.48 (m, 4H), 1.46 (br s, 3H)

### Step 8: methyl (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylate

To a solution of methyl (1R,4r)-4-(8-(hydroxymethyl)-6-((R)-3-methyl-3-phenylpyrrolidin-1-yl)-[1,2,4]triazolo[1,5-a]pyridine-2-carboxamido)cyclohexane-1-carboxylate (82 mg, 166.81 μmol, 1 eq) in DCM (1 mL) was added SOCl₂ (59.54 mg, 500.43 μmol, 36.35 μL, 3 eq) at 0° C. The mixture was stirred at 25° C. for 0.5 h. LCMS showed starting material consumed. One main peak was shown on LCMS and desired compound was detected. The reaction mixture was concentrated under reduced pressure to give a residue. The residue was purified by flash silica gel chromatography (SiO₂, PE:EtOAc=1:1, Rf=0.5) to give compound methyl (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylate (66 mg, 72.61% yield) as a red solid.

LC-MS [ESI, M+1]: 510.2.

### Step 9: (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylic acid

To a solution of methyl (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylate (65 mg, 127.44 μmol, 1 eq) in MeOH (1 mL) and H₂O (0.3 mL) was added LiOH·H₂O (16.04 mg, 382.33 μmol, 3 eq). The mixture was stirred at 25° C. for 0.5 h. LCMS showed starting material consumed. Several peaks were shown on LCMS and desired compound was detected. The reaction was adjusted pH to 7 with FA (0.1 mL) and then was concentrated under reduced pressure to give a residue. The crude product was purified by reversed-phase HPLC (0.1% FA condition) to give compound (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylic acid (35 mg, 48.39% yield) as a white solid.

LC-MS [ESI, M+1]: 496.2.

### Step 10: (1R,4r)-4-(8-(2-ethoxy-2-oxoethyl)-6-((R)-3-methyl-3-phenylpyrrolidin-1-yl)-[1,2,4]triazolo[1,5-a]pyridine-2-carboxamido)cyclohexane-1-carboxylic acid

To a solution of (1R,4r)-4-(8-(chloromethyl)-6-((R)-3-methyl-3-phenylpyrrolidin-1-yl)-[1,2,4]triazolo[1,5-a]pyridine-2-carboxamido)cyclohexane-1-carboxylic acid (35 mg, 70.56 μmol, 1 eq) in EtOH (5 mL) was added Pd(dppf)C12 (5.16 mg, 7.06 μmol, 0.1 eq) and TEA (21.42 mg, 211.69 μmol, 29.47 μL, 3 eq) under N₂ atmosphere. The suspension was degassed and purged with CO for 3 times. The mixture was stirred under CO (50 Psi) at 80° C. for 12 h. LCMS showed starting material consumed. One main peak was shown on LCMS and desired compound was detected. The reaction mixture was filtered and concentrated under reduced pressure to give a residue. The crude product was purified by reversed-phase HPLC (0.1% FA condition) to give compound (1R,4r)-4-(8-(2-ethoxy-2-oxoethyl)-6-((R)-3-methyl-3-phenylpyrrolidin-1-yl)-[1,2,4]triazolo[1,5-a]pyridine-2-carboxamido)cyclohexane-1-carboxylic acid (20 mg, 40.37% yield) as white solid.

LC-MS [ESI, M+1]: 534.2

### Step 11: (1R,4r)-4-(8-(2-amino-2-oxoethyl)-6-((R)-3-methyl-3-phenylpyrrolidin-1-yl)-[1,2,4]triazolo[1,5-a]pyridine-2-carboxamido)cyclohexane-1-carboxylic acid

A mixture of (1R,4r)-4-(8-(2-ethoxy-2-oxoethyl)-6-((R)-3-methyl-3-phenylpyrrolidin-1-yl)-[1,2,4]triazolo[1,5-a]pyridine-2-carboxamido)cyclohexane-1-carboxylic acid (20 mg, 37.48 μmol, 1 eq) in NH₃/MeOH (1 mL) was degassed and purged with N₂ for 3 times, and then the mixture was stirred at 50° C. for 0.5 h under N₂ atmosphere. LCMS showed starting material consumed. Several peaks were shown on LCMS and desired compound was detected. The reaction mixture was filtered and concentrated under reduced pressure to give a residue. The residue was purified by prep-HPLC (FA condition; column: CD01-Phenomenex luna C18 150×25 mm×10 um; mobile phase: [H₂O (0.225% FA)—ACN]; gradient: 32%-62% B over 11.0 min) to give compound (1R,4r)-4-(8-(2-amino-2-oxoethyl)-6-((R)-3-methyl-3-phenylpyrrolidin-1-yl)-[1,2,4]triazolo[1,5-a]pyridine-2-carboxamido)cyclohexane-1-carboxylic acid (2.48 mg, 12.96% yield) as a yellow solid.

LC-MS [ESI, M+1]: 505.2.

¹H NMR (400 MHz, CHLOROFORM-d) δ=7.67 (br s, 1H), 7.35 (br d, J=6.8 Hz, 2H), 7.30 (br s, 1H), 7.25 (br s, 2H), 7.16 (br s, 2H), 6.21 (br s, 1H), 3.94 (br s, 3H), 3.68-3.33 (m, 4H), 2.46-1.98 (m, 7H), 1.61 (br d, J=7.2 Hz, 2H), 1.44 (br s, 3H), 1.35 (br s, 2H).

| Compound | | |
|---|---|---|
| Number | Synthesis | LC/MS |
| 758 | Example 138 | m/z 504.59 [M + H]+ |

### Example 139

### 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetic acid (Compound 759)

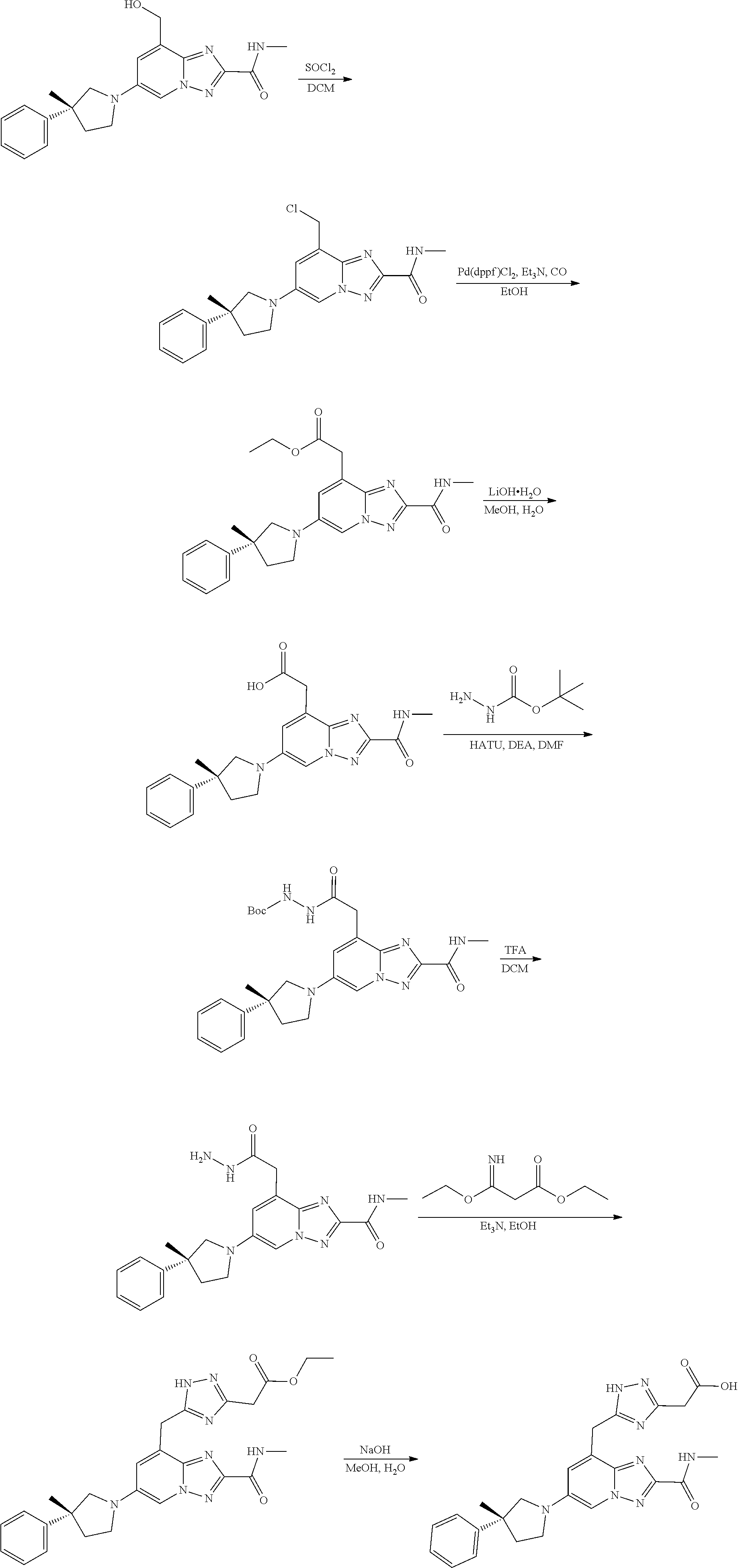

### Step 1: 8-(chloromethyl)-N-methyl-6-[(3R)-3-methyl-3-phenyl-pyrrolidin-1-yl]-[1,2,4]triazolo[1,5-a]pyridine-2-carboxamide

To a solution of 8-(hydroxymethyl)-N-methyl-6-[(3R)-3-methyl-3-phenyl-pyrrolidin-1-yl]-[1,2,4]triazolo[1,5-a]pyridine-2-carboxamide (240 mg, 656.76 μmol, 1 eq) in DCM (3 mL) was added SOCl₂ (234.41 mg, 1.97 mmol, 143.10 μL, 3 eq) at 0° C. The mixture was stirred at 25° C. for 0.5 h. LCMS showed starting material was consumed and one main peak with desired mass was detected. The reaction mixture was adjusted pH around 7 with saturated NaHCO₃, then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.68) to give compound 8-(chloromethyl)-N-methyl-6-[(3R)-3-methyl-3-phenyl-pyrrolidin-1-yl]-[1,2,4]triazolo[1,5-a]pyridine-2-carboxamide (220 mg, 87% yield) as white solid.

LC-MS [ESI, M+1]: 384.2.

¹H NMR (400 MHz, CHLOROFORM-d) δ=7.77 (d, J=2.0 Hz, 1H), 7.39 (d, J=7.2 Hz, 2H), 7.37-7.29 (m, 4H), 7.28 (s, 1H), 4.98 (s, 2H), 3.65 (d, J=8.8 Hz, 1H), 3.62-3.51 (m, 2H), 3.47 (dt, J=3.6, 8.8 Hz, 1H), 3.10 (d, J=5.2 Hz, 3H), 2.43 (td, J=8.4, 12.4 Hz, 1H), 2.31 (dt, J=3.6, 7.6 Hz, 1H), 1.50 (s, 3H).

### Step 2: ethyl 2-[2-(methylcarbamoyl)-6-[(3R)-3-methyl-3-phenyl-pyrrolidin-1-yl]-[1,2,4]triazolo[1,5-a]pyridin-8-yl]acetate

To a solution of 8-(chloromethyl)-N-methyl-6-[(3R)-3-methyl-3-phenyl-pyrrolidin-1-yl]-[1,2,4]triazolo[1,5-a]pyridine-2-carboxamide (220.00 mg, 573.11 μmol, 1 eq) in EtOH (10 mL) was added Pd(dppf)C12 (46.80 mg, 57.31 μmol, 0.1 eq) and Et₃N (115.98 mg, 1.15 mmol, 159.54 μL, 2 eq). The suspension was degassed and purged with CO for 3 times. The mixture was stirred under CO (50 Psi) at 80° C. for 12 h. LCMS showed starting material was consumed and one main peak with desired mass was detected. The reaction mixture was filtered and the filtrate was concentrated under reduced pressure to give a residue. The residue was purified by column chromatography (SiO₂, hexanes:EtOAc=0:1, Rf=0.45) to give compound ethyl 2-[2-(methylcarbamoyl)-6-[(3R)-3-methyl-3-phenyl-pyrrolidin-1-yl]-[1,2,4]triazolo[1,5-a]pyridin-8-yl]acetate (200 mg, 83% yield) as white solid.

LC-MS [ESI, M+1]: 422.2.

¹H NMR (400 MHz, CHLOROFORM-d) δ=7.73 (s, 1H), 7.36 (br d, J=7.2 Hz, 2H), 7.34-7.28 (m, 3H), 7.25 (br s, 1H), 7.18 (s, 1H), 4.22 (q, J=7.2 Hz, 2H), 4.02 (s, 2H), 3.61 (br d, J=8.8 Hz, 1H), 3.59-3.47 (m, 2H), 3.47-3.39 (m, 1H), 3.07 (d, J=5.2 Hz, 3H), 2.39 (td, J=8.4, 12.0 Hz, 1H), 2.32-2.21 (m, 1H), 1.47 (s, 3H), 1.29 (t, J=7.2 Hz, 3H).

### Step 3: 2-[2-(methylcarbamoyl)-6-[(3R)-3-methyl-3-phenyl-pyrrolidin-1-yl]-[1,2,4]triazolo[1,5-a]pyridin-8-yl]acetic acid

To a solution of ethyl 2-[2-(methylcarbamoyl)-6-[(3R)-3-methyl-3-phenyl-pyrrolidin-1-yl]-[1,2,4]triazolo[1,5-a]pyridin-8-yl]acetate (75 mg, 177.94 μmol, 1 eq) in THF (1 mL) was added LiOH·H₂O (14.93 mg, 355.88 μmol, 2 eq) and H₂O (0.3 mL). The mixture was stirred at 25° C. for 0.5 h. LCMS showed starting material was consumed and one main peak with desired mass was detected. The reaction mixture was adjusted pH around 7 with 2 M HCl, then extracted with ethyl acetate (10 mL×3), the combined organic layers dried over anhydrous sodium sulfate, filtered and the filtrate was concentrated under reduced pressure to give a residue. The residue was purified by column chromatography (SiO₂, DCM:MeOH=10:1, Rf=0.38) to give compound 2-[2-(methylcarbamoyl)-6-[(3R)-3-methyl-3-phenyl-pyrrolidin-1-yl]-[1,2,4]triazolo[1,5-a]pyridin-8-yl]acetic acid (70 mg, 99% yield) as brown solid.

LC-MS [ESI, M+1]: 394.1.

### Step 4: tert-butyl N-[[2-[2-(methylcarbamoyl)-6-[(3R)-3-methyl-3-phenyl-pyrrolidin-1-yl]-[1,2,4]triazolo[1,5-a]pyridin-8-yl]acetyl]amino]carbamate

To a solution of 2-[2-(methylcarbamoyl)-6-[(3R)-3-methyl-3-phenyl-pyrrolidin-1-yl]-[1,2,4]triazolo[1,5-a]pyridin-8-yl]acetic acid (55 mg, 139.79 μmol, 1 eq) and tert-butyl N-aminocarbamate (22.17 mg, 167.75 μmol, 1.2 eq) in DMF (2 mL) was added HATU (79.73 mg, 209.69 μmol, 1.5 eq) and DIEA (36.13 mg, 279.59 μmol, 48.70 μL, 2 eq). The mixture was stirred at 25° C. for 0.5 h. LCMS showed starting material was consumed and the desired mass was detected. The reaction mixture was quenched by H₂O (3 mL), then extracted with ethyl acetate (10 mL×3), the combined organic layers dried over anhydrous sodium sulfate, filtered and the filtrate was concentrated under reduced pressure to give a residue. The residue was purified by column chromatography (SiO₂, hexanes:EtOAc=0:1, Rf=0.50) to give compound tert-butyl N-[[2-[2-(methylcarbamoyl)-6-[(3R)-3-methyl-3-phenyl-pyrrolidin-1-yl]-[1,2,4]triazolo[1,5-a]pyridin-8-yl]acetyl]amino]carbamate (65 mg, 91% yield) as yellow solid.

LC-MS [ESI, M+1]: 508.3.

¹H NMR (400 MHz, CHLOROFORM-d) δ=9.74-9.39 (m, 1H), 7.69 (br s, 2H), 7.41-7.34 (m, 2H), 7.34-7.27 (m, 3H), 7.25 (br s, 1H), 6.83 (br dd, J=2.4, 4.4 Hz, 1H), 3.95 (br s, 2H), 3.61 (br d, J=8.4 Hz, 1H), 3.58-3.47 (m, 2H), 3.43 (br d, J=4.4 Hz, 1H), 3.08 (br d, J=2.4 Hz, 3H), 2.44-2.33 (m, 1H), 2.32-2.22 (m, 1H), 1.55-1.37 (m, 12H).

### Step 5: 8-(2-hydrazino-2-oxo-ethyl)-N-methyl-6-[(3R)-3-methyl-3-phenyl-pyrrolidin-1-yl]-[1,2,4]triazolo[1,5-a]pyridine-2-carboxamide

To a solution of tert-butyl N-[[2-[2-(methylcarbamoyl)-6-[(3R)-3-methyl-3-phenyl-pyrrolidin-1-yl]-[1,2,4]triazolo[1,5-a]pyridin-8-yl]acetyl]amino]carbamate (325 mg, 640.29 μmol, 1 eq) in DCM (5 mL) was added TFA (730.08 mg, 6.40 μmol, 65.86 μL, 10 eq). The mixture was stirred at 25° C. for 1 h. LCMS showed starting material was consumed and the desired mass was detected. The reaction was concentrated under reduced pressure to give a residue. The residue was purified by reversed-phase HPLC purification (mobile phase: [water(HCl)—ACN]; B %: 12%-42%, 14 min), the mixture was concentrated and lyophilized to give compound 8-(2-hydrazino-2-oxo-ethyl)-N-methyl-6-[(3R)-3-methyl-3-phenyl-pyrrolidin-1-yl]-[1,2,4]triazolo[1,5-a]pyridine-2-carboxamide (255 mg, 97% yield) as brown solid.

LC-MS [ESI, M+1]: 408.1.

¹H NMR (400 MHz, METHANOL-d4) δ=7.87 (br s, 1H), 7.44 (br s, 1H), 7.42-7.26 (m, 4H), 7.25-7.18 (m, 1H), 4.03 (br s, 2H), 3.71-3.47 (m, 3H), 3.45-3.36 (m, 1H), 2.98 (br s, 3H), 2.49-2.17 (m, 2H), 1.60-1.31 (m, 3H).

### Step 6: ethyl 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetate

To a solution of 8-(2-hydrazino-2-oxo-ethyl)-N-methyl-6-[(3R)-3-methyl-3-phenyl-pyrrolidin-1-yl]-[1,2,4]triazolo[1,5-a]pyridine-2-carboxamide (245 mg, 601.27 μmol, 1 eq) and ethyl 3-ethoxy-3-imino-propanoate (191.42 mg, 1.20 mmol, 2 eq) in EtOH (10 mL) was added Et₃N (182.53 mg, 1.80 mmol, 251.07 μL, 3 eq). The mixture was stirred at 80° C. for 12 h. LCMS showed starting material was consumed and the desired mass was detected. The reaction was concentrated under reduced pressure to give a residue. The residue was purified by reversed-phase HPLC purification (mobile phase: [water(FA)—ACN]; B %: 29%-59%, 10 min), the mixture was concentrated and lyophilized to give compound ethyl 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetate (100 mg, 33% yield) as yellow solid.

LC-MS [ESI, M+1]: 503.3.

¹H NMR (400 MHz, DMSO-d₆) δ=13.57 (br s, 1H), 8.50 (br s, 1H), 7.97 (s, 1H), 7.46-7.31 (m, 5H), 7.28-7.19 (m, 1H), 4.37 (br s, 2H), 4.07 (q, J=7.2 Hz, 2H), 3.70 (br d, J=5.6 Hz, 2H), 3.61-3.55 (m, 1H), 3.54-3.46 (m, 2H), 3.37 (br s, 1H), 2.80 (d, J=4.8 Hz, 3H), 2.33-2.17 (m, 2H), 1.36 (s, 3H), 1.15 (t, J=7.2 Hz, 3H).

### Step 7: e 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetic acid

To a solution of ethyl 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetate (80 mg, 159.18 μmol, 1 eq) in MeOH (2 mL) was added NaOH (12.73 mg, 318.37 μmol, 2 eq) and H₂O (0.5 mL). The mixture was stirred at 25° C. for 0.5 h. LCMS showed starting material was consumed and the desired mass was detected. The reaction was concentrated under reduced pressure to give a residue. The residue was purified by reversed-phase HPLC purification (mobile phase: [water(FA)—ACN]; B %: 20%-50%, 13 min), the mixture was concentrated and lyophilized to give compound 2-[5-[[2-(methylcarbamoyl)-6-[(3R)-3-methyl-3-phenyl-pyrrolidin-1-yl]-[1,2,4]triazolo[1,5-a]pyridin-8-yl]methyl]-1H-1,2,4-triazol-3-yl]acetic acid (41.76 mg, 53% yield) as yellow solid.

LC-MS [ESI, M+1]: 475.3.

¹H NMR (400 MHz, DMSO-d₆) δ=8.50 (br d, J=3.2 Hz, 1H), 7.97 (s, 1H), 7.48-7.29 (m, 5H), 7.24 (br d, J=7.2 Hz, 1H), 4.35 (br s, 2H), 3.64 (br s, 2H), 3.57 (br d, J=9.6 Hz, 1H), 3.50 (br dd, J=4.0, 9.2 Hz, 3H), 2.80 (br d, J=4.8 Hz, 3H), 2.26 (br t, J=6.0 Hz, 2H), 1.36 (s, 3H).

### Example 140

### (R)-8-(difluoromethyl)-N-methyl-6-(3-methyl-3-phenylpyrrolidin-1-yl)-[1,2,4]triazolo[1,5-a]pyridine-2-carboxamide (Compound 760)

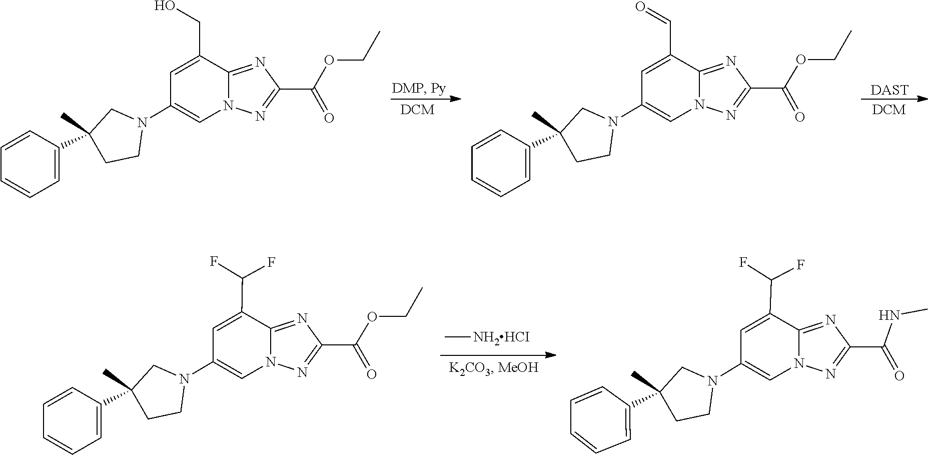

### Step 1: ethyl (R)-8-formyl-6-(3-methyl-3-phenylpyrrolidin-1-yl)-[1,2,4]triazolo[1,5-a]pyridine-2-carboxylate

To a solution of ethyl 8-(hydroxymethyl)-6-[(3R)-3-methyl-3-phenyl-pyrrolidin-1-yl]-[1,2,4]triazolo[1,5-a]pyridine2-carboxylate (100 mg, 262.85 μmol, 1 eq) in DCM (2 mL) was added Pyridine (41.58 mg, 525.71 μmol, 42.43 μL, 2 eq) and (1,1-diacetoxy-3-oxo-1,2-benziodoxol-1-yl) acetate (222.97 mg, 525.71 μmol, 162.87 μL, 2 eq). The mixture was stirred at 0° C. for 1 h. LCMS showed starting material was consumed and the desired mass was detected. The mixture was quenched with water (10 mL) and extracted with ethyl acetate (20 mL×2). The combined organic phase was washed with brine (30 mL), dried with anhydrous Na₂SO₄, filtered and concentrated in vacuum. The residue was purified by column chromatography (SiO₂, PE:EtOAc=1:1, Rf=0.5) to give compound ethyl 8-formyl-6-[(3R)-3-methyl-3-phenyl-pyrrolidin-1-yl]-[1,2,4]triazolo[1,5-a]pyridine-2-carboxylate (99 mg, 235.45 μmol, 89.57% yield, 90% purity) as yellow solid.

LC-MS [ESI, M+23]: 379.2.

¹H NMR (400 MHz, CHLOROFORM-d) δ=10.81 (s, 1H), 8.05 (d, J=2.4 Hz, 1H), 7.84 (d, J=2.4 Hz, 1H), 7.42-7.35 (m, 2H), 7.34-7.28 (m, 3H), 4.58 (q, J=7.2 Hz, 2H), 3.69 (d, J=9.2 Hz, 1H), 3.64-3.55 (m, 2H), 3.49 (dt, J=3.6, 8.8 Hz, 1H), 2.45 (td, J=8.4, 12.4 Hz, 1H), 2.37-2.28 (m, 1H), 1.54-1.45 (m, 6H).

### Step 2: ethyl (R)-8-(difluoromethyl)-6-(3-methyl-3-phenylpyrrolidin-1-yl)-[1,2,4]triazolo[1,5-a]pyridine-2-carboxylate

To a solution of ethyl 8-formyl-6-[(3R)-3-methyl-3-phenyl-pyrrolidin-1-yl]-[1,2,4]triazolo[1,5-a]pyridine-2-carboxylate (90 mg, 237.83 μmol, 1 eq) in DCM (1 mL) was added DAST (95.84 mg, 594.57 μmol, 78.56 μL, 2.5 eq). The mixture was stirred at 0° C. for 10 min. LCMS showed starting material was consumed and the desired mass was detected. The PH was adjusted to around 7 by progressively aq. NaHCO₃, and then extracted with DCM (10 mL×2). The combined organic layers were washed with brine (10 mL), dried over Na₂SO₄, filtered and concentrated under reduced pressure to give a residue. The residue was purified by column chromatography (SiO₂, PE:EtOAc=1:1, Rf=0.50) to give compound ethyl 8-(difluoromethyl)-6-[(3R)-3-methyl-3-phenyl-pyrrolidin-1-yl]-[1,2,4]triazolo[1,5-a]pyridine-2-carboxylate (80 mg, 189.80 μmol, 79.81% yield, 95% purity) as yellow oil.

LC-MS [ESI, M+1]: 401.2.

¹H NMR (400 MHz, CHLOROFORM-d) δ=7.89 (s, 1H), 7.54 (d, J=1.2 Hz, 1H), 7.47 (s, 0.2H), 7.42-7.36 (m, 2H), 7.35-7.28 (m, 3H), 7.20 (s, 0.8H), 4.56 (q, J=7.2 Hz, 2H), 3.66 (d, J=8.8 Hz, 1H), 3.62-3.52 (m, 2H), 3.47 (dt, J=3.6, 8.8 Hz, 1H), 2.44 (td, J=8.4, 12.2 Hz, 1H), 2.36-2.21 (m, 1H), 1.54-1.44 (m, 6H).

### Step 3: (R)-8-(difluoromethyl)-N-methyl-6-(3-methyl-3-phenylpyrrolidin-1-yl)-[1,2,4]triazolo[1,5-a]pyridine-2-carboxamide

To a solution of ethyl 8-(difluoromethyl)-6-[(3R)-3-methyl-3-phenyl-pyrrolidin-1-yl]-[1,2,4]triazolo[1,5-a]pyridine-2-carboxylate (40 mg, 99.89 μmol, 1 eq) in MeOH (1 mL) was added methanamine;hydrochloride (67.45 mg, 998.95 μmol, 10 eq) and K₂CO₃ (69.03 mg, 499.47 μmol, 5 eq). The mixture was stirred at 70° C. for 1 h. LCMS showed starting material was consumed and the desired mass was detected. The reaction mixture was filtered and the filtrate was concentrated under the reduced pressure to give the product. The residue was purified by prep-HPLC (FA condition column: CD01-Phenomenex luna C18 150*25*10 um; mobile phase: [H₂O (0.225% FA)—ACN]; gradient:38%-68% B over 10.0 min) to give compound 8-(difluoromethyl)-N-methyl-6-[(3R)-3-methyl-3-phenyl-pyrrolidin-1-yl]-[1,2,4]triazolo[1,5-a]pyridine-2-carboxamide (21.58 mg, 55.55 μmol, 55.61% yield, 99.209% purity) as yellow solid.

LC-MS [ESI, M+1]: 386.2.

¹H NMR (400 MHz, CHLOROFORM-d) δ=7.87 (s, 1H), 7.49 (s, 1H), 7.42-7.28 (m, 6H), 7.26-7.04 (m, 1H), 3.66 (d, J=8.9 Hz, 1H), 3.62-3.51 (m, 2H), 3.47 (dt, J=3.6, 8.8 Hz, 1H), 3.08 (d, J=5.2 Hz, 3H), 2.43 (td, J=8.4, 12.3 Hz, 1H), 2.36-2.25 (m, 1H), 1.49 (s, 3H).

### Example 141

### 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoic acid

### Step 1: methyl 5-[4-(tert-butoxycarbonylamino)cyclohexen-1-yl]-2-fluoro-benzoate (Compound 761)

A mixture of tert-butyl N-[4-(4,4,5,5-tetramethyl-1,3,2-dioxaborolan-2-yl)cyclohex-3-en-1-yl]carbamate (1 g, 3.09 mmol, 1 eq), methyl 5-bromo-2-fluoro-benzoate (720.95 mg, 3.09 mmol, 1 eq), Na₂CO₃ (655.80 mg, 6.19 mmol, 2 eq) and cyclopentyl(diphenyl)phosphane;dichloromethane;dichloropalladium;iron (252.65 mg, 309.37 μmol, 0.1 eq) in dioxane (10 mL) and H₂O (1 mL) was degassed and purged with N₂ for 3 times, and then the mixture was stirred at 100° C. for 12 h under N₂ atmosphere. LCMS showed starting material was consumed. Several new peaks were shown on LCMS and 75.94% of desired compound was detected. The reaction mixture was filtered and concentrated under reduced pressure to give a residue. The residue was purified by flash silica gel chromatography (SiO₂, PE:EtOAc=2:1, Rf=0.4) to give compound methyl 5-[4-(tert-butoxycarbonylamino)cyclohexen-1-yl]-2-fluoro-benzoate (824 mg, 75.51% yield) as white solid.

LC-MS [ESI, M+23]: 372.1.

### Step 2: methyl 5-[4-(tert-butoxycarbonylamino)cyclohexyl]-2-fluoro-benzoate

To a solution of methyl 5-[4-(tert-butoxycarbonylamino)cyclohexen-1-yl]-2-fluoro-benzoate (300 mg, 858.63 μmol, 1 eq) in EtOH (20 mL) was added Pd/C (91.37 mg, 85.86 μmol, 10% purity, 0.1 eq) under N₂ atmosphere. The suspension was degassed and purged with H₂ for 3 times. The mixture was stirred under H₂ (15 Psi) at 25° C. for 12 h. LCMS showed starting material was consumed. One main peak was shown on LCMS and 98.36% of desired compound was detected. The reaction mixture was filtered and concentrated under reduced pressure to give a crude methyl 5-[4-(tert-butoxycarbonylamino)cyclohexyl]-2-fluoro-benzoate (303 mg, 98.77% yield) as white solid.

LC-MS [ESI, M+23]: 374.0.

¹H NMR (400 MHz, CHLOROFORM-d) δ=7.76 (ddd, J=2.4, 6.9, 13.4 Hz, 1H), 7.41-7.30 (m, 1H), 7.06 (ddd, J=5.2, 8.6, 10.4 Hz, 1H), 4.94-4.32 (m, 1H), 3.93 (d, J=5.2 Hz, 3H), 3.72 (q, J=7.2 Hz, 1H), 2.67-2.43 (m, 1H), 2.14 (br d, J=10.4 Hz, 1H), 1.97-1.86 (m, 2H), 1.80-1.51 (m, 5H), 1.47 (d, J=6.0 Hz, 9H)

### Step 3: methyl 5-(4-aminocyclohexyl)-2-fluoro-benzoate

To a solution of methyl 5-[4-(tert-butoxycarbonylamino)cyclohexyl]-2-fluoro-benzoate (303 mg, 862.24 μmol, 1 eq) in DCM (1.5 mL) was added HCl/dioxane (1.5 mL) at 0° C. The resulting mixture was stirred at 25° C. for 1 h. LCMS showed starting material was consumed and one main peak with desired mass was detected. The reaction mixture was concentrated under reduced pressure to give a crude methyl 5-(4-aminocyclohexyl)-2-fluoro-benzoate (220 mg, 98.46% yield) as a white solid.

LC-MS [ESI, M+1]: 252.1.

### Step 4: methyl 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoate

To a solution of 8-methyl-6-[(3R)-3-methyl-3-phenyl-pyrrolidin-1-yl]-[1,2,4]triazolo[1,5-a]pyridine-2-carboxylic acid (35 mg, 104.05 μmol, 1 eq) in DMF (0.5 mL) was added methyl 5-(4-aminocyclohexyl)-2-fluoro-benzoate (52.29 mg, 208.09 μmol, 2 eq), HATU (59.34 mg, 156.07 μmol, 1.5 eq) and DIEA (26.89 mg, 208.09 μmol, 36.25 μL, 2 eq). The resulting mixture was stirred at 25° C. for 1 h. LCMS showed starting material was consumed. Several new peaks were shown on LCMS and 51.35% of desired compound was detected. The reaction mixture was diluted with H₂O 15 mL and extracted with EtOAc (15 mL×3). The combined organic layers were washed with brine 75 mL (15 mL×5), dried over Na₂SO₄, filtered and concentrated under reduced pressure to give a crude methyl 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoate (30 mg, 26.99% yield) as yellow oil.

LC-MS [ESI, M+1]: 570.2.

### Step 5: 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoic acid

To a solution of methyl 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoate (30 mg, 52.66 μmol, 1 eq) in MeOH (0.6 mL) and H₂O (0.3 mL) was added LiOH·H₂O (4.42 mg, 105.32 μmol, 2 eq). The mixture was stirred at 25° C. for 2 h. LCMS showed starting material was consumed competely. Several new peaks were shown on LCMS and 51.08% of desired compound was detected. The mixture was adjusted pH to 5 with HCl (6 M) and then was filtered to give a residue. The residue was purified by prep-HPLC (FA condition; column: Phenomenex luna C18 150*25 mm*10 um; mobile phase: [water(FA)—ACN]; gradient: 53%-83% B over 10 min) to give compound 2-fluoro-5-[4-[[8-methyl-6-[(3R)-3-methyl-3-phenyl-pyrrolidin-1-yl]-[1,2,4]triazolo[1,5-a]pyridine-2-carbonyl]amino]cyclohexyl]benzoic acid (13.38 mg, 44.24% yield) as a white solid.

LC-MS [ESI, M+1]: 556.2.

¹H NMR (400 MHz, CHLOROFORM-d) δ=7.91 (br d, J=6.8 Hz, 1H), 7.69 (s, 1H), 7.59 (br d, J=7.6 Hz, 1H), 7.50-7.41 (m, 1H), 7.40-7.34 (m, 2H), 7.34-7.30 (m, 2H), 7.28 (br s, 1H), 7.10 (br t, J=9.6 Hz, 1H), 7.01 (s, 1H), 4.47 (br s, 1H), 3.61 (d, J=8.8 Hz, 1H), 3.58-3.47 (m, 2H), 3.42 (dt, J=3.6, 8.7 Hz, 1H), 2.66 (s, 4H), 2.42-2.35 (m, 1H), 2.31-2.24 (m, 1H), 2.13-2.05 (m, 2H), 1.88-1.74 (m, 6H), 1.47 (s, 3H)

| Compound | | |
|---|---|---|
| Number | Synthesis | LC/MS |
| 761 | Example 141 | m/z 555.69 [M + H]⁺ |

### Example 142

### Differential Scanning Fluorimetry Assay

The stabilization effects the compounds have on the immunoglobulin light chain protein was quantitively measured using a thermal shift assay. One or both of the following assays could be used.

Recombinant amyloidogenic full length light chain protein WIL-FL T46L was produced in E. co/i, purified, and used in the assay. The Thermal Shift Protein Stability Kit from Biotium was used and the assay was done in 96-well Plates following the manufacturer's instructions. The protein and compounds were incubated at RT for 30 min before adding the dye and subjected to the thermal shift assay. The thermal stability (Tm) of each sample was then determined using standard curve fitting methods and compared to vehicle. Data are expressed as temperature shift: [Tm]_{compound}-[Tm]_{vehicle}.

Recombinant amyloidogenic full length light chain protein WIL-FL T46L was produced in Expi293 cells, purified, and used in the assay. The Thermal Shift Protein Stability Kit from Biotium was used and the assay was done in 384-well Plates following the manufacturer's instructions. The protein and compounds were incubated at RT for 30 min before adding the dye and subjected to the thermal shift assay. The thermal stability (Tm) of each sample was then determined using standard curve fitting methods and compared to vehicle. Data are expressed as temperature shift: [Tm]_{compound}-[Tm]_{vehicle}.

### Results

The compounds provided herein induce shifts in the ranges (0-0.5° C.=*; >0.5-1° C. =**; >1-1.5° C.=***; >1.5-2.5° C.=****; >2.5° C.=*****). Results for the compounds provided herein are shown in Table A and Table B.

TABLE A

| Cmpd No. | DSF |
|---|---|
| 1A | ***** |
| 1B | ***** |
| 2A | ***** |
| 2B | ***** |
| 3 | ***** |
| 4 | ***** |
| 5 | ***** |
| 6 | ***** |
| 7 | ***** |
| 8 | ***** |
| 9 | ***** |
| 10 | ***** |
| 11 | ***** |
| 12 | ***** |
| 13 | ***** |
| 14 | ** |
| 15 | ***** |
| 16 | * |
| 17 | ***** |
| 18 | ***** |
| 19 | ***** |
| 20 | ***** |
| 21 | *** |
| 22 | **** |
| 23 | **** |
| 24A | ***** |
| 24B | ***** |
| 25 | * |
| 26 | *** |
| 27 | ***** |
| 28 | **** |
| 29 | ***** |
| 30 | ***** |
| 31 | ***** |
| 32 | ***** |
| 33 | ***** |
| 34 | **** |
| 35 | *** |
| 36 | ***** |
| 37 | ***** |
| 38 | * |
| 39 | ***** |
| 40 | ***** |
| 41 | ***** |
| 42 | ***** |
| 43 | ***** |
| 44 | ***** |
| 45 | ***** |
| 46 | ***** |
| 47 | ***** |
| 48 | ***** |
| 49 | ***** |
| 50 | ***** |
| 51 | ** |
| 52 | ***** |
| 53 | ***** |
| 54 | ***** |
| 55 | ***** |
| 56 | ***** |
| 57 | ***** |
| 58 | ***** |
| 59 | ***** |
| 60 | ***** |
| 61 | **** |
| 62 | ***** |
| 63 | ***** |
| 64 | ***** |
| 65 | **** |
| 66 | ***** |
| 67 | **** |
| 68 | **** |
| 69 | **** |
| 70 | ***** |
| 71 | ***** |
| 72 | ***** |
| 73 | ***** |
| 74 | ***** |
| 75 | ***** |
| 76 | ***** |
| 77 | ***** |
| 78 | ***** |
| 79 | ***** |
| 80 | ***** |
| 81 | ***** |
| 82 | ***** |
| 83 | **** |
| 84 | ***** |
| 85 | ***** |
| 86 | ***** |
| 87 | ***** |
| 88 | ***** |
| 89 | ***** |
| 90 | **** |
| 91 | ***** |
| 92 | ***** |
| 93 | ***** |
| 94 | ***** |
| 95 | ***** |
| 96 | **** |
| 97 | ***** |
| 98 | *** |
| 99 | **** |
| 100 | ***** |
| 101 | ***** |
| 102 | ***** |
| 103 | ***** |
| 104 | ***** |
| 105 | ***** |
| 106 | ***** |
| 107 | ***** |
| 108 | ***** |
| 109 | ***** |
| 110 | ***** |
| 111 | ***** |
| 112 | ***** |
| 113 | ***** |
| 114 | ***** |
| 115 | ***** |
| 116 | ***** |
| 117 | ***** |
| 118 | ***** |
| 119 | ***** |
| 120 | ***** |
| 121 | ***** |
| 122 | ***** |
| 123 | ***** |
| 124 | ***** |
| 125 | ***** |
| 126 | ***** |
| 127 | ***** |
| 128 | ***** |
| 129 | ***** |
| 130 | ***** |
| 131 | ***** |
| 132 | ***** |
| 133 | ***** |
| 134 | ***** |
| 135 | ***** |
| 136 | ***** |
| 137 | ***** |
| 138 | ***** |
| 139 | ***** |
| 140 | ***** |
| 141 | ***** |
| 142 | ***** |
| 143 | ***** |
| 144 | ***** |
| 145 | ***** |
| 146 | ***** |
| 147 | ***** |
| 148 | ***** |
| 149 | ***** |
| 150 | ***** |
| 151 | ***** |
| 152 | ***** |
| 153 | ***** |
| 154 | ***** |
| 155 | ***** |
| 156 | ***** |
| 157 | ***** |
| 158 | ***** |
| 159 | ***** |
| 160 | ***** |
| 161 | ***** |
| 162 | ***** |
| 163 | ***** |
| 164 | ***** |
| 165 | ***** |
| 166 | ***** |
| 167 | ***** |
| 168 | **** |
| 169 | ***** |
| 170 | ***** |
| 171 | ***** |
| 172 | ***** |
| 173 | ***** |
| 174 | ***** |
| 175 | ***** |
| 176 | ***** |
| 177 | ***** |
| 178 | ***** |
| 179 | ***** |
| 180 | ***** |
| 181 | ***** |
| 182 | ***** |
| 183 | ***** |
| 184 | ** |
| 185 | ***** |
| 186 | ***** |
| 187 | ***** |
| 188 | ***** |
| 189 | ***** |
| 190 | ***** |
| 191 | ***** |
| 192 | ***** |
| 193 | ***** |
| 194 | ***** |
| 195 | ***** |
| 196 | ***** |
| 197 | ***** |
| 198 | ***** |
| 199 | ***** |
| 200 | ***** |
| 201 | ***** |
| 202 | ***** |
| 203 | ***** |
| 204 | ***** |
| 205 | ***** |
| 206 | ***** |
| 207 | ***** |
| 208 | ***** |
| 209 | ***** |
| 210 | ***** |
| 211 | ***** |
| 212 | ***** |
| 213 | ***** |
| 214 | ***** |
| 215 | ***** |
| 216 | ***** |
| 217 | ***** |
| 218 | ***** |
| 219 | ***** |
| 220 | ***** |
| 221 | ***** |
| 222 | ***** |
| 223 | ***** |
| 224 | ***** |
| 225 | ***** |
| 226 | ***** |
| 227 | ***** |
| 228 | ***** |
| 229 | ***** |
| 230 | ***** |
| 231 | ***** |
| 232 | ***** |
| 233 | ***** |
| 234 | ***** |
| 235 | ***** |
| 236 | ***** |
| 237 | ***** |
| 238 | ***** |
| 239 | ***** |
| 240 | ***** |
| 241 | ***** |
| 242 | ***** |
| 243 | ***** |
| 244 | ***** |
| 245 | ***** |
| 246 | ***** |
| 247 | ***** |
| 248 | ***** |
| 249 | ***** |
| 250 | ***** |
| 251 | ***** |
| 252 | ***** |
| 253 | ***** |
| 254 | ***** |
| 255 | ***** |
| 256 | ***** |
| 257 | ***** |
| 258 | ***** |
| 259 | ***** |
| 260 | ***** |
| 261 | ***** |
| 262 | ***** |
| 263 | ***** |
| 264 | ***** |
| 265 | ***** |
| 266 | ***** |
| 267 | ***** |
| 268 | ***** |
| 269 | ***** |
| 270 | ***** |
| 271 | ***** |
| 272 | ***** |
| 273 | ***** |
| 274 | ***** |
| 275 | ***** |
| 276 | ***** |
| 277 | ***** |
| 278 | ***** |
| 279 | ***** |
| 280 | ***** |
| 281 | ***** |
| 282 | ***** |
| 283 | ***** |
| 284 | ***** |
| 285 | ***** |
| 286 | ***** |
| 287 | ***** |
| 288 | ***** |
| 289 | ***** |
| 290 | ***** |
| 291 | ***** |
| 292 | *** |
| 293 | **** |
| 294 | ***** |
| 295 | * |
| 296 | ** |
| 297 | * |
| 298 | ** |
| 299 | *** |
| 300 | *** |
| 301 | * |
| 302 | **** |
| 303 | ***** |
| 304 | ***** |
| 305 | ***** |
| 306 | ***** |
| 307 | ***** |
| 308 | ***** |
| 309 | **** |
| 310 | ***** |
| 311 | ***** |
| 312 | **** |
| 313 | ***** |
| 314 | ***** |
| 315 | **** |
| 316 | **** |
| 317 | ***** |
| 318 | ***** |
| 319 | ***** |
| 320 | ***** |
| 321 | ***** |
| 322 | ***** |
| 323 | ***** |
| 324 | ***** |
| 325 | ***** |
| 326 | ***** |
| 327 | ***** |
| 328 | **** |
| 329 | ***** |
| 330 | **** |
| 331 | ** |
| 332 | ***** |
| 333 | ***** |
| 334 | ***** |
| 335 | ***** |
| 336 | ***** |
| 337 | **** |
| 338 | * |
| 339 | ***** |
| 340 | ***** |
| 341 | ***** |
| 342 | ***** |
| 343 | ***** |
| 344 | ***** |
| 345 | ***** |
| 346 | ***** |
| 347 | ***** |
| 348 | ***** |
| 349 | ***** |
| 350 | ***** |
| 351 | ***** |
| 352 | ***** |
| 353 | ***** |
| 354 | ***** |
| 355 | ***** |
| 356 | ***** |
| 357 | ***** |
| 358 | ***** |
| 359 | ***** |
| 360 | ***** |
| 361 | ***** |
| 362 | ***** |
| 363 | ***** |
| 364 | ***** |
| 365 | ***** |
| 366 | ***** |
| 367 | ***** |
| 368 | ***** |
| 369 | ***** |
| 370 | ***** |
| 371 | ***** |
| 372 | ***** |
| 373 | ***** |
| 374 | ***** |
| 375 | ***** |
| 376 | ***** |
| 377 | ***** |
| 378 | ***** |
| 379 | ***** |
| 380 | ***** |
| 381 | ***** |
| 382 | ***** |
| 383 | ***** |
| 384 | ***** |
| 385 | ***** |
| 386 | ** |
| 387 | ** |
| 388 | **** |
| 389 | ***** |
| 390 | ***** |
| 391 | ***** |
| 392 | ***** |
| 393 | ***** |
| 394 | ***** |
| 395 | ***** |
| 396 | ***** |
| 397 | ***** |
| 398 | ***** |
| 399 | ***** |
| 400 | ** |
| 401 | * |
| 402 | ***** |
| 403 | ***** |
| 404 | ***** |
| 405 | ***** |
| 406 | ***** |
| 407 | ***** |
| 408 | ***** |
| 409 | ***** |
| 410 | *** |
| 411 | ***** |
| 412 | * |
| 413 | **** |
| 414 | ***** |
| 415 | ***** |
| 416 | ***** |
| 417 | ***** |
| 418 | ***** |
| 419 | ***** |
| 420 | ***** |
| 421 | ***** |
| 422 | ***** |
| 423 | *** |
| 424 | **** |
| 425 | ***** |
| 426 | ***** |
| 427 | *** |
| 428 | ***** |
| 429 | ***** |
| 430 | * |
| 431 | * |
| 432 | * |
| 433 | ***** |
| 434 | ***** |
| 435 | ***** |
| 436 | ***** |
| 437 | *** |
| 438 | ***** |
| 439 | ***** |

TABLE B

| Cmpd No. | DSF |
|---|---|
| 440 | ***** |
| 441 | ***** |
| 442 | ***** |
| 443 | ***** |
| 444 | ***** |
| 445 | ***** |
| 446 | ***** |
| 447 | ***** |
| 448 | ***** |
| 449 | ***** |
| 450 | ***** |
| 451 | ***** |
| 452 | ***** |
| 453 | ***** |
| 454 | ***** |
| 455 | ***** |
| 456 | ***** |
| 457 | ***** |
| 458 | ***** |
| 459 | ***** |
| 460 | ***** |
| 461 | ***** |
| 462 | ***** |
| 463 | ***** |
| 464 | ***** |
| 465 | ***** |
| 466 | ***** |
| 467 | ***** |
| 468 | ***** |
| 469 | ***** |
| 470 | ***** |
| 471 | **** |
| 472 | ***** |
| 473 | ***** |
| 474 | **** |
| 475 | ***** |
| 476 | ***** |
| 477 | ***** |
| 478 | ***** |
| 479 | ***** |
| 480 | ***** |
| 481 | ***** |
| 482 | ***** |
| 483 | ***** |
| 484 | ***** |
| 485 | ***** |
| 486 | **** |
| 487 | *** |
| 488 | ***** |
| 489 | ***** |
| 490 | ***** |
| 491 | ***** |
| 492 | ***** |
| 493 | ***** |
| 494 | ***** |
| 495 | ***** |
| 496 | **** |
| 497 | ***** |
| 498 | ***** |
| 499 | ***** |
| 500 | ***** |
| 501 | ***** |
| 502 | ***** |
| 503 | ***** |
| 504 | ***** |
| 505 | ***** |
| 506 | ***** |
| 507 | ***** |
| 508 | ***** |
| 509 | ***** |
| 510 | ***** |
| 511 | ***** |
| 512 | ***** |
| 513 | ***** |
| 514 | |
| 515 | ***** |
| 516 | ***** |
| 517 | ***** |
| 518 | ***** |
| 519 | ***** |
| 520 | ***** |
| 521 | ***** |
| 522 | ***** |
| 523 | ***** |
| 524 | ***** |
| 525 | ***** |
| 526 | ***** |
| 527 | ***** |
| 528 | ***** |
| 529 | ***** |
| 530 | ***** |
| 531 | ***** |
| 532 | ***** |
| 533 | ***** |
| 534 | **** |
| 535 | ***** |
| 536 | ***** |
| 537 | **** |
| 538 | ***** |
| 539 | ***** |
| 540 | ***** |
| 541 | ***** |
| 542 | ***** |
| 543 | ***** |
| 544 | ***** |
| 545 | ***** |
| 546 | ***** |
| 547 | ***** |
| 548 | ***** |
| 549 | ***** |
| 550 | ***** |
| 551 | ***** |
| 552 | ***** |
| 553 | ***** |
| 554 | ***** |
| 555 | ***** |
| 556 | ***** |
| 557 | ***** |
| 558 | ***** |
| 559 | ***** |
| 560 | ***** |
| 561 | ***** |
| 562 | ***** |
| 563 | **** |
| 564 | ***** |
| 565 | ***** |
| 566 | ***** |
| 567 | **** |
| 568 | ***** |
| 569 | **** |
| 570 | ***** |
| 571 | ***** |
| 572 | ***** |
| 573 | ***** |
| 574 | ***** |
| 575 | ***** |
| 576 | ***** |
| 577 | ***** |
| 578 | ***** |
| 579 | **** |
| 580 | ***** |
| 581 | ***** |
| 582 | ***** |
| 583 | ***** |
| 584 | ***** |
| 585 | ***** |
| 586 | ***** |
| 587 | ***** |
| 588 | ***** |
| 589 | ***** |
| 590 | ***** |
| 591 | ***** |
| 592 | ***** |
| 593 | ***** |
| 594 | ***** |
| 595 | ***** |
| 596 | ***** |
| 597 | ***** |
| 598 | ***** |
| 599 | ***** |
| 600 | ***** |
| 601 | ***** |
| 602 | ***** |
| 603 | ***** |
| 604 | ***** |
| 605 | ***** |
| 606 | ***** |
| 607 | ***** |
| 608 | ***** |
| 609 | ***** |
| 610 | ***** |
| 611 | ***** |
| 612 | ***** |
| 613 | ***** |
| 614 | ***** |
| 615 | **** |
| 616 | ***** |
| 617 | ***** |
| 618 | ***** |
| 619 | ***** |
| 620 | ***** |
| 621 | ***** |
| 622 | ***** |
| 623 | ***** |
| 624 | ***** |
| 625 | ***** |
| 626 | ***** |
| 627 | ***** |
| 628 | ***** |
| 629 | ***** |
| 630 | ***** |
| 631 | ***** |
| 632 | ***** |
| 633 | ***** |
| 634 | ***** |
| 635 | ***** |
| 636 | ***** |
| 637 | ***** |
| 638 | ***** |
| 639 | ***** |
| 640 | ***** |
| 641 | ***** |
| 642 | ***** |
| 643 | ***** |
| 644 | ***** |
| 645 | ***** |
| 646 | ***** |
| 647 | ***** |
| 648 | ***** |
| 649 | ***** |
| 650 | ***** |
| 651 | ***** |
| 652 | ***** |
| 653 | ***** |
| 654 | ***** |
| 655 | ***** |
| 656 | ***** |
| 657 | ***** |
| 658 | ***** |
| 659 | ***** |
| 660 | ***** |
| 661 | ***** |
| 662 | ***** |
| 663 | ***** |
| 664 | ***** |
| 665 | ***** |
| 666 | ***** |
| 667 | ***** |
| 668 | ***** |
| 669 | ***** |
| 670 | ***** |
| 671 | ***** |
| 672 | ***** |
| 673 | ***** |
| 674 | ***** |
| 675 | ***** |
| 676 | ***** |
| 677 | ***** |
| 678 | ***** |
| 679 | ***** |
| 680 | ***** |
| 681 | ***** |
| 682 | ***** |
| 683 | ***** |
| 684 | ***** |
| 685 | ***** |
| 686 | ***** |
| 687 | ***** |
| 688 | ***** |
| 689 | ***** |
| 690 | ***** |
| 691 | ***** |
| 692 | ***** |
| 693 | ***** |
| 694 | ***** |
| 695 | ***** |
| 696 | ***** |
| 697 | ***** |
| 698 | ***** |
| 699 | ***** |
| 700 | ***** |
| 701 | ***** |
| 702 | ***** |
| 703 | ***** |
| 704 | ***** |
| 705 | ***** |
| 706 | ***** |
| 707 | ***** |
| 708 | ***** |
| 709 | ***** |
| 710 | ***** |
| 711 | ***** |
| 712 | ***** |
| 713 | ***** |
| 714 | ***** |
| 715 | ***** |
| 716 | ***** |
| 717 | ***** |
| 718 | ***** |
| 719 | ***** |
| 720 | ***** |
| 721 | ***** |
| 722 | ***** |
| 723 | ***** |
| 724 | ***** |
| 725 | ***** |
| 726 | ***** |
| 727 | ***** |
| 728 | ***** |
| 729 | ***** |
| 730 | ***** |
| 731 | ***** |
| 732 | ***** |
| 733 | ***** |
| 734 | ***** |
| 735 | **** |
| 736 | ***** |
| 737 | ***** |
| 738 | ***** |
| 739 | ***** |
| 740 | **** |
| 741 | ***** |
| 742 | ***** |
| 743 | ***** |
| 744 | ***** |
| 745 | *** |
| 746 | ***** |
| 747 | ***** |
| 748 | ***** |
| 749 | ***** |
| 750 | ***** |
| 751 | ***** |
| 752 | ***** |
| 753 | ***** |
| 754 | *** |
| 755 | ***** |
| 756 | **** |
| 758 | ***** |
| 759 | **** |
| 760 | ***** |
| 761 | ***** |
| 762 | ***** |
| 763 | ***** |

### EQUIVALENTS

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

1.-71. (canceled)

72. A compound of Formula (VIII): 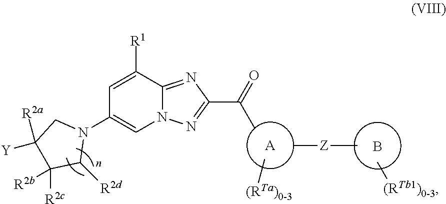
or a pharmaceutically acceptable salt thereof, wherein:
n is 0 or 1;
R¹ is halogen, cyano, —OH, —O(C₁-C₆ alkyl), —NH₂, —NH(C₁-C₆ alkyl), —N(C₁-C₆ alkyl)₂, or C₁-C₆ alkyl, wherein the C₁-C₆ alkyl is optionally substituted with one or more halogen, cyano, —OH, —NH₂, —NHC(═O)(C₁-C₆ alkyl), —NHS(═O)₂(C₁-C₆ alkyl), or —C(═O)NH₂;
R^{2a} is C₁-C₆ alkyl optionally substituted with one or more halogen, cyano, —OH, or —NH₂;
R^{2b} is H or C₁-C₆ alkyl, wherein the C₁-C₆ alkyl is optionally substituted with one or more halogen;
R^{2c} is H or C₁-C₆ alkyl, wherein the C₁-C₆ alkyl is optionally substituted with one or more halogen;
R^{2d} is H; or R^{2a} and R^{2d} together form C₁-C₆ alkylene optionally substituted with one or more halogen;
Y is C₃-C₈ cycloalkyl, C₆-C₁₀ aryl, or 5- to 10-membered heteroaryl, wherein the C₃-C₈ cycloalkyl, C₆-C₁₀ aryl, or 5- to 10-membered heteroaryl is optionally substituted with one or more R^{Ya};
each R^{Ya} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{Yb};
each R^{Yb} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), or —C(═O)—N(C₁-C₆ alkyl)₂;
ring A is 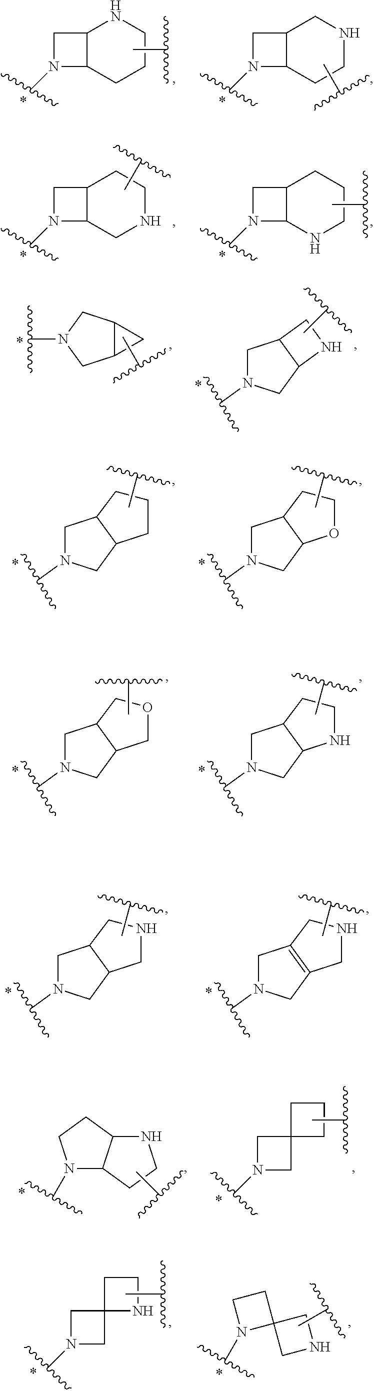 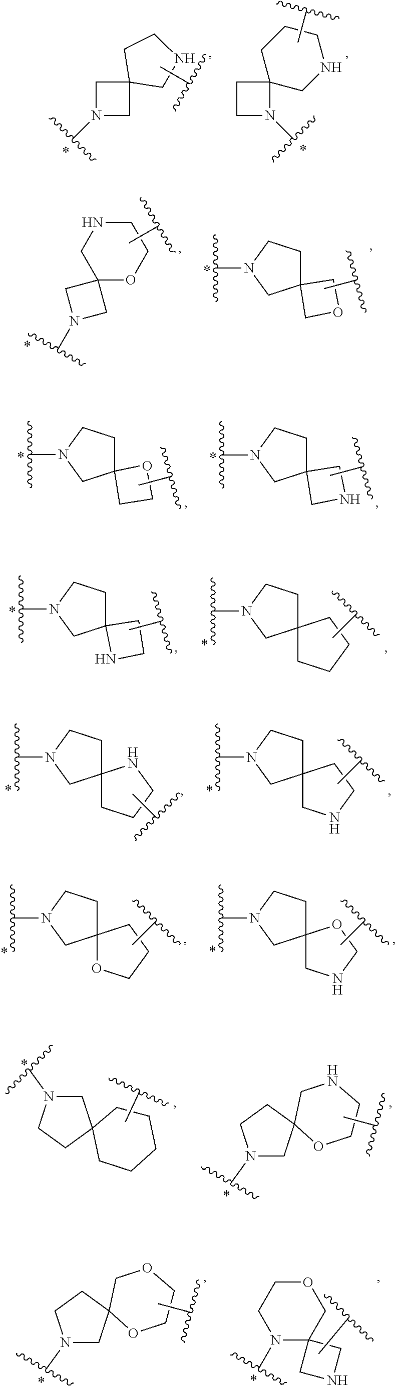 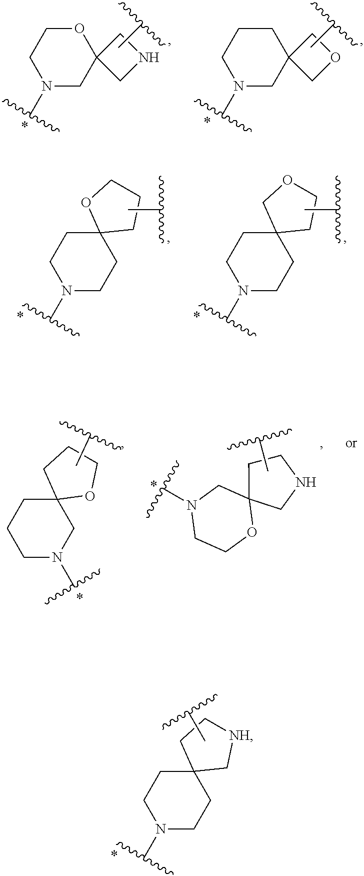
where * indicates connectivity to the carbonyl group at triazolopyridine;
ring B is H, C₃-C₆ cycloalkyl, 4- to 10-membered heterocyclyl, C₆-C₁₀ aryl, or 5- to 10-membered heteroaryl;
Z is absent, —CH₂-, —O—, or —C(═O)—;
each R^{Ta} independently is oxo, halogen, cyano, —OH, —OR^{Tb}, —NH₂, —NHR^{Tb}, —N(R^{Tb})₂, —C(═O)—H, —C(═O)—R^{Tb}, —C(═O)—OH, —C(═O)—OR^{Tb}, —C(═O)—NH₂, —C(═O)—NHR^{Tb}, —C(═O)—N(R^{Tb})₂, —S(═O)₂—R^{Tb}, —S(═O)(═NH)—R^{Tb}, C₁-C₆ alkyl, C₁-C₆ haloalkyl, C₂-C₆ alkenyl, C₂-C₆ haloalkenyl, C₂-C₆ alkynyl, or C₂-C₆ alkynyl;
each R^{Tb} is C₁-C₆ alkyl, C₁-C₆ haloalkyl, C₂-C₆ alkenyl, C₂-C₆ haloalkenyl, C₂-C₆ alkynyl, or C₂-C₆ alkynyl; and
each R^{Tb1} independently is oxo, halogen, cyano, —OH, —O(C₁-C₆ alkyl), —NH₂, —NH(C₁-C₆ alkyl), —N(C₁-C₆ alkyl)₂, —C(═O)—H, —C(═O)—OH, —C(═O)—O(C₁-C₆ alkyl), —C(═O)—NH₂, —C(═O)—NH(C₁-C₆ alkyl), —C(═O)—N(C₁-C₆ alkyl)₂, C₁-C₆ alkyl, C₂-C₆ alkenyl, or C₂-C₆ alkynyl;
wherein when B is H, Z is absent.

73. The compound of claim 72, or a pharmaceutically acceptable salt thereof, wherein the compound has the structure of Formula (VIII-a), or (VIII-b):

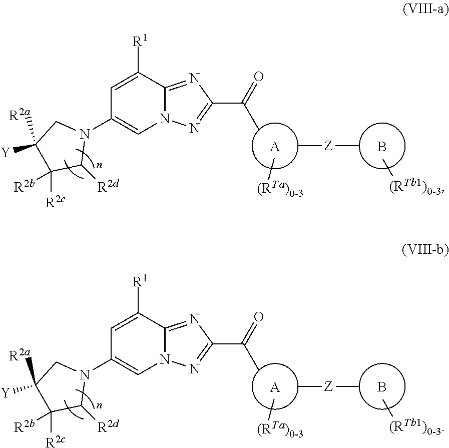

74. The compound of claim 72, or a pharmaceutically acceptable salt thereof, wherein Y is phenyl or 5- to 6-membered heteroaryl, wherein the phenyl or the 5- to 10-membered heteroaryl is optionally substituted with one or more R^{Ya}.

75. The compound of claim 72, or a pharmaceutically acceptable salt thereof, wherein Y is phenyl, pyrrolyl, furanyl, thiophenyl, oxazolyl, thiazolyl, pyrazolyl, isoxazolyl, isothiazolyl, imidazolyl, triazolyl, oxadiazolyl, thiadiazolyl, oxathiadiazolyl, tetrazolyl, oxatriazolyl, pyridinyl, pyranyl, thiopyranyl, pyrimidinyl, pyridazinyl, or pyrazinyl.

76. The compound of claim 72, or a pharmaceutically acceptable salt thereof, wherein Y is phenyl.

77. The compound of claim 72, or a pharmaceutically acceptable salt thereof, wherein R¹ is C₁-C₃ alkyl optionally substituted with one or more of halogen, cyano, —OH, —NH₂, —NHC(═O)(C₁-C₆ alkyl), —NHS(═O)₂(C₁-C₆ alkyl), or —C(═O)NH₂.

78. The compound of claim 77, or a pharmaceutically acceptable salt thereof, wherein R¹ is methyl.

79. The compound of claim 72, or a pharmaceutically acceptable salt thereof, wherein R^{2a} is C₁-C₃ alkyl.

80. The compound of claim 72, or a pharmaceutically acceptable salt thereof, wherein R^{2b}, R^{2c}, and R^{2d} are each H.

81. The compound of claim 72, or a pharmaceutically acceptable salt thereof, wherein ring A is: 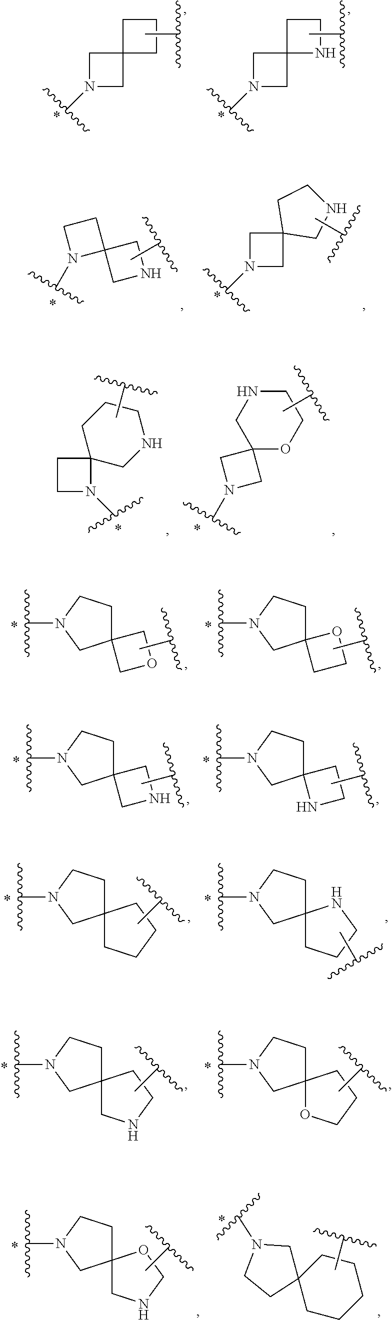 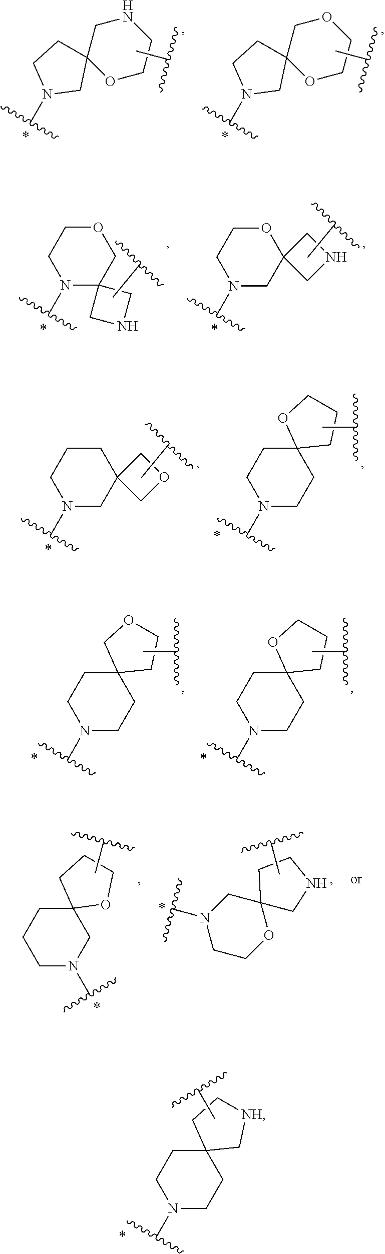
where * indicates connectivity to the carbonyl group at triazolopyridine.

82. The compound of claim 81, or a pharmaceutically acceptable salt thereof, wherein ring A is: 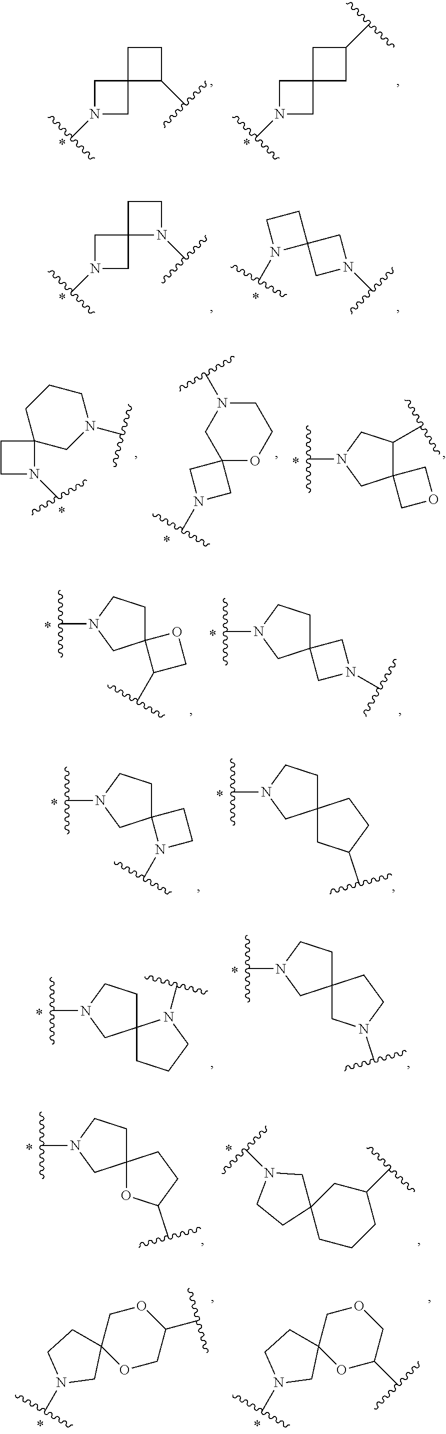 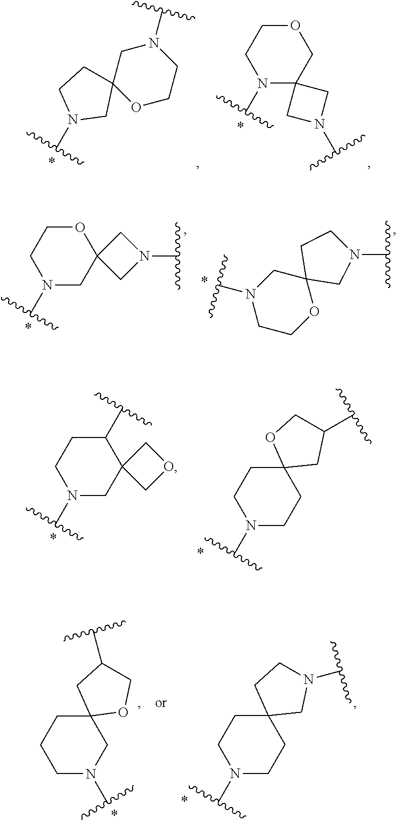
where * indicates connectivity to the carbonyl group at triazolopyridine.

83. The compound of claim 72, or a pharmaceutically acceptable salt thereof, wherein ring B is C₃-C₆ cycloalkyl, 4- to 7-membered heterocyclyl, phenyl, or 5- to 6-membered heteroaryl.

84. The compound of claim 83, or a pharmaceutically acceptable salt thereof, wherein ring B is pyridinyl, pyranyl, thiopyranyl, pyrimidinyl, pyridazinyl, or pyrazinyl.

85. The compound of claim 83, or a pharmaceutically acceptable salt thereof, wherein ring B is pyridinyl.

86. The compound of claim 72, or a pharmaceutically acceptable salt thereof, wherein at least one R^{Tb} is present and the at least one R^{Tb1} is —C(═O)—H, —C(═O)—OH, or —C(═O)—O(C₁-C₆ alkyl).

87. The compound of claim 72, or a pharmaceutically acceptable salt thereof, wherein n is 1.

88. The compound of claim 72, or a pharmaceutically acceptable salt thereof, wherein Z is absent.

89. The compound of claim 72, or a pharmaceutically acceptable salt thereof, wherein there is no R^{Ta} and there is zero, one, or two R^{Tb1}.

90. The compound of claim 72, or a pharmaceutically acceptable salt thereof, wherein:
R¹ is C₁-C₃ alkyl;
R^{2a} is C₁-C₃ alkyl; and
R^{2b}, R^{2c}, and R^{2d} are each H or C₁-C₃ alkyl.

91. The compound of claim 72, or a pharmaceutically acceptable salt thereof, wherein
ring A is 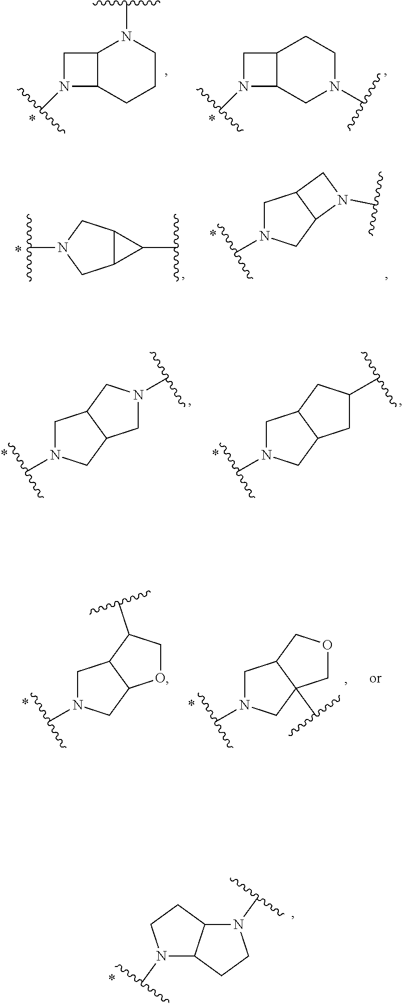
where * indicates connectivity to the carbonyl group at triazolopyridine;
ring B is pyridinyl, pyranyl, thiopyranyl, pyrimidinyl, pyridazinyl, or pyrazinyl; and
at least one R^{Tb1} is present and the at least one R^{Tb1} is —C(═O)—H, —C(═O)—OH, or —C(═O)—O(C₁-C₆ alkyl).

92. The compound of claim 72, or a pharmaceutically acceptable salt thereof, wherein:
ring A is 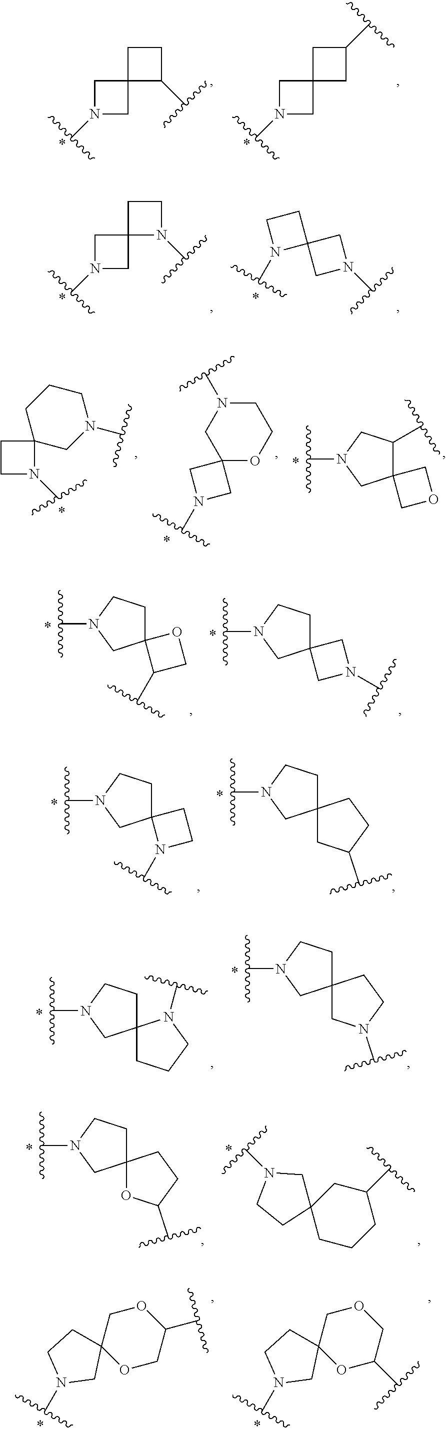 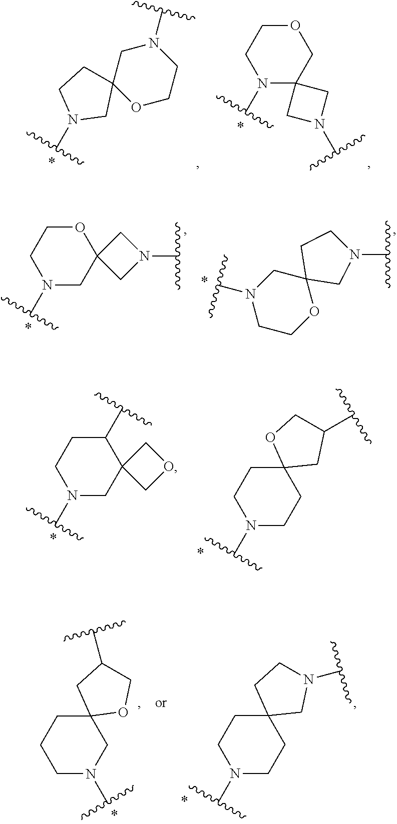
where * indicates connectivity to the carbonyl group at triazolopyridine;
ring B is pyridinyl, pyranyl, thiopyranyl, pyrimidinyl, pyridazinyl, or pyrazinyl; and
at least one R^{Tb1} is present and the at least one R^{Tb1} is —C(═O)—H, —C(═O)—OH, or —C(═O)—O(C₁-C₆ alkyl).

93. A compound selected from: 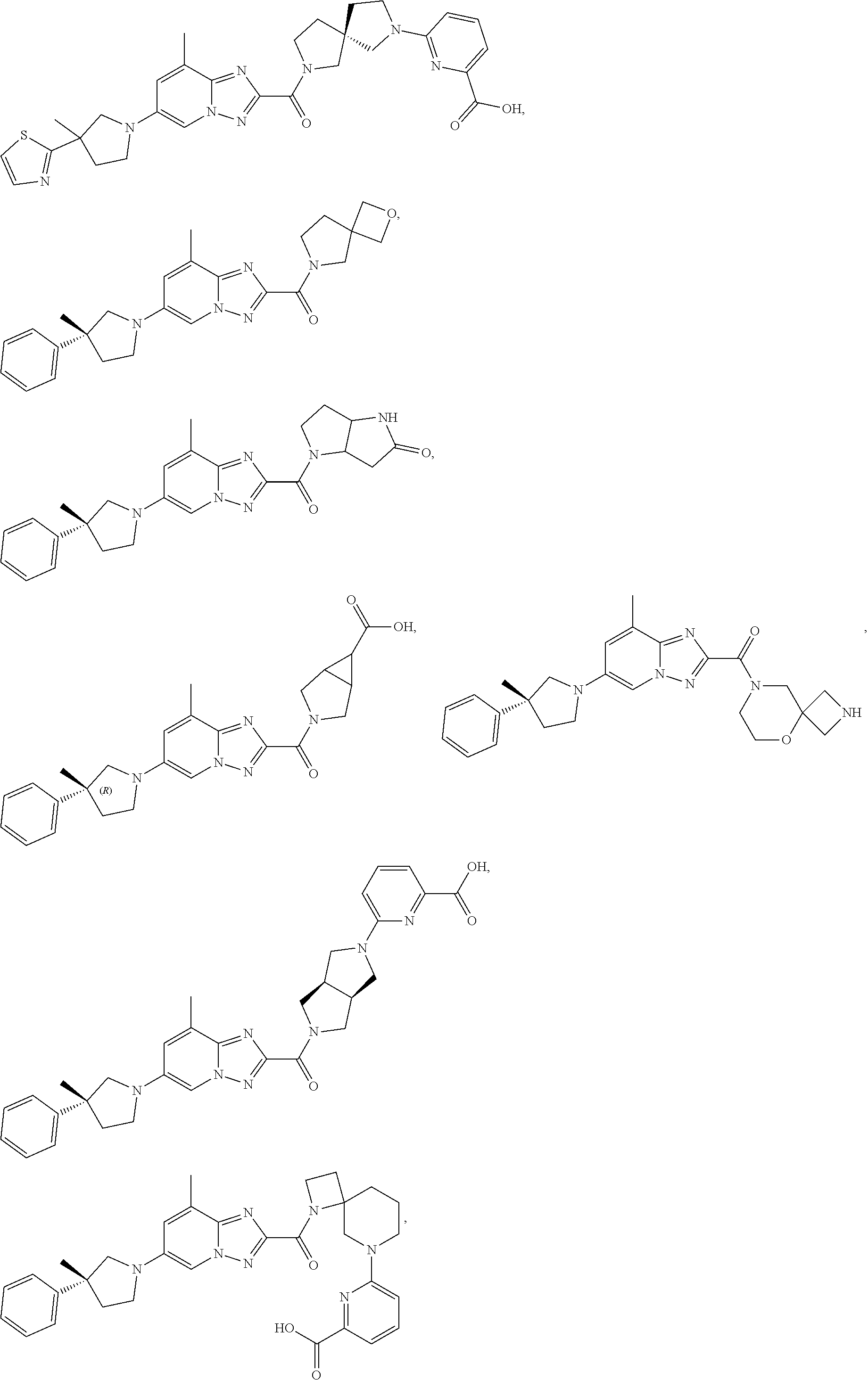 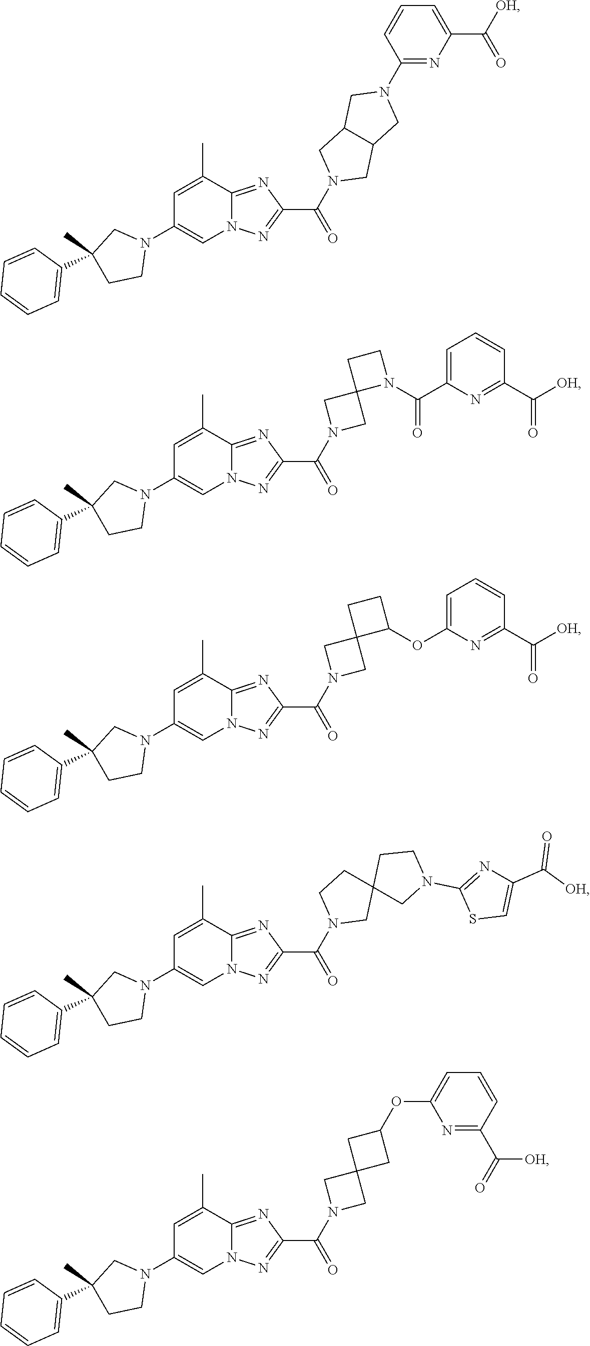 [SMILES: Cc1cc(N2CC[C@](C)(c3ccccc3)C2)cn2nc(C(=O)N3CC4CN(c5cccc(C(=O)O)n5)CC4C3)nc12 ; Cc1cc(N2CC[C@](C)(c3ccccc3)C2)cn2nc(C(=O)N3CC4(CCN4C(=O)c4cccc(C(=O)O)n4)C3)nc12 ; Cc1cc(N2CC[C@](C)(c3ccccc3)C2)cn2nc(C(=O)N3CC4(CCC4Oc4cccc(C(=O)O)n4)C3)nc12 ; Cc1cc(N2CC[C@](C)(c3ccccc3)C2)cn2nc(C(=O)N3CCC4(CCN(c5nc(C(=O)O)cs5)C4)C3)nc12 ; Cc1cc(N2CC[C@](C)(c3ccccc3)C2)cn2nc(C(=O)N3CC4(CC(Oc5cccc(C(=O)O)n5)C4)C3)nc12] 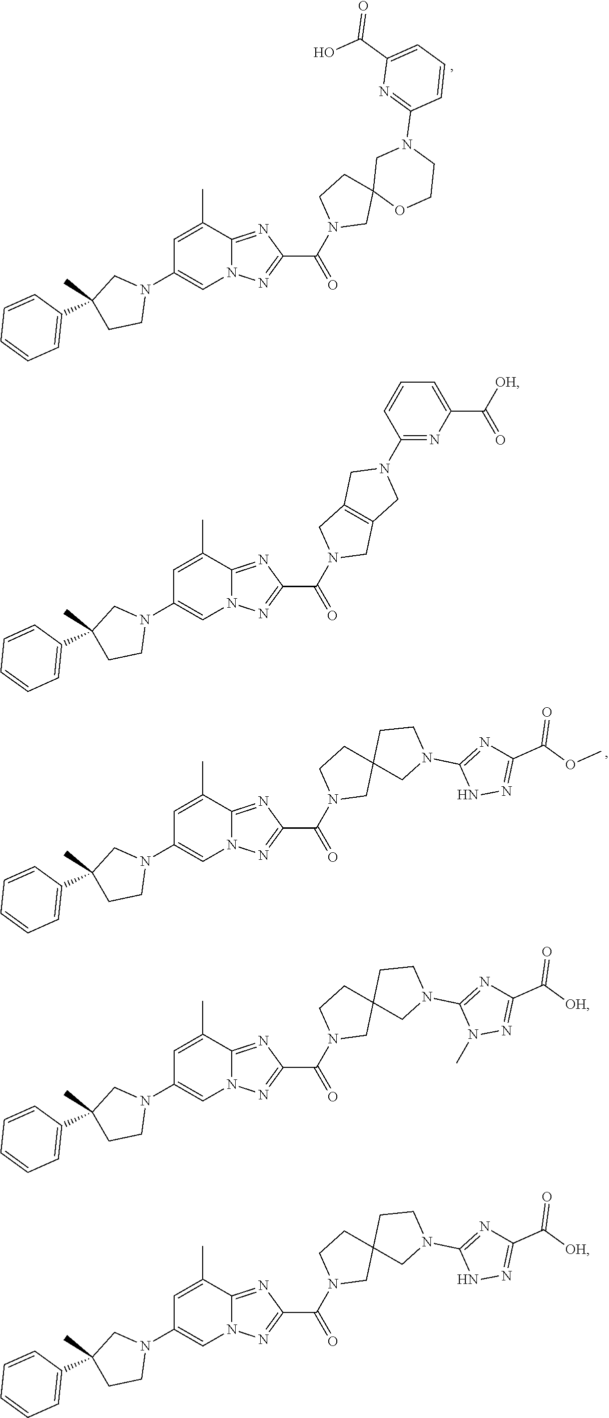 [SMILES: Cc1cc(N2CC[C@](C)(c3ccccc3)C2)cn2nc(C(=O)N3CCC4(C3)CN(c3cccc(C(=O)O)n3)CCO4)nc12 ; Cc1cc(N2CC[C@](C)(c3ccccc3)C2)cn2nc(C(=O)N3CC4=C(C3)CN(c3cccc(C(=O)O)n3)C4)nc12 ; COC(=O)c1n[nH]c(N2CCC3(CCN(C(=O)c4nc5c(C)cc(N6CC[C@](C)(c7ccccc7)C6)cn5n4)C3)C2)n1 ; Cc1cc(N2CC[C@](C)(c3ccccc3)C2)cn2nc(C(=O)N3CCC4(CCN(c5nc(C(=O)O)nn5C)C4)C3)nc12 ; Cc1cc(N2CC[C@](C)(c3ccccc3)C2)cn2nc(C(=O)N3CCC4(CCN(c5nc(C(=O)O)n[nH]5)C4)C3)nc12] 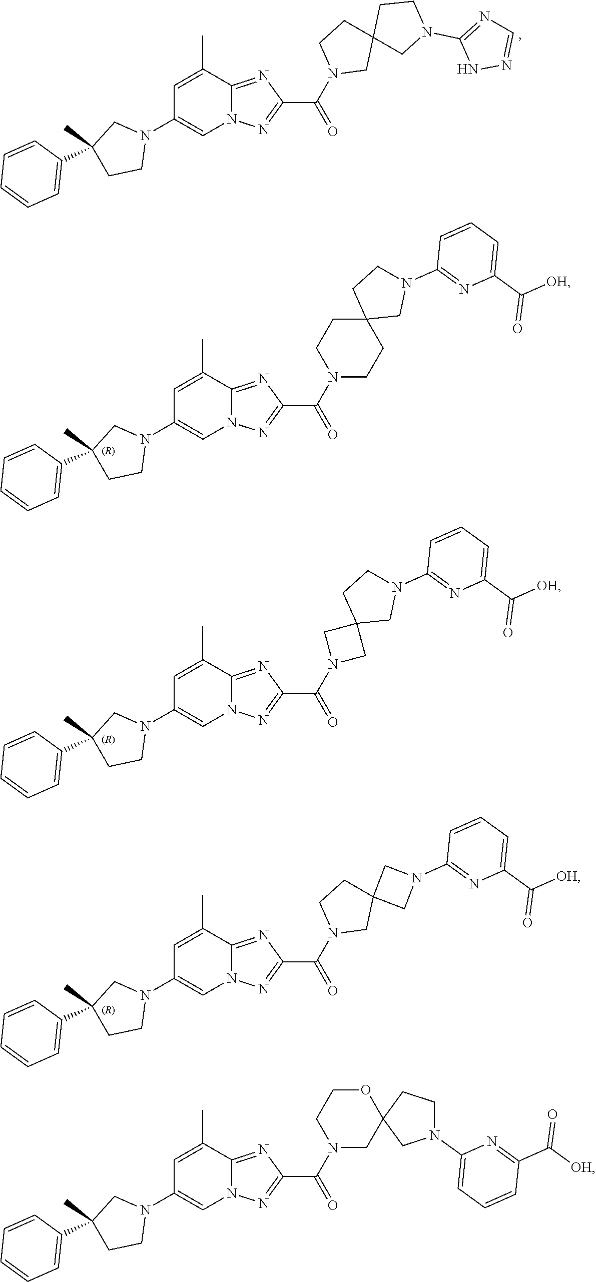 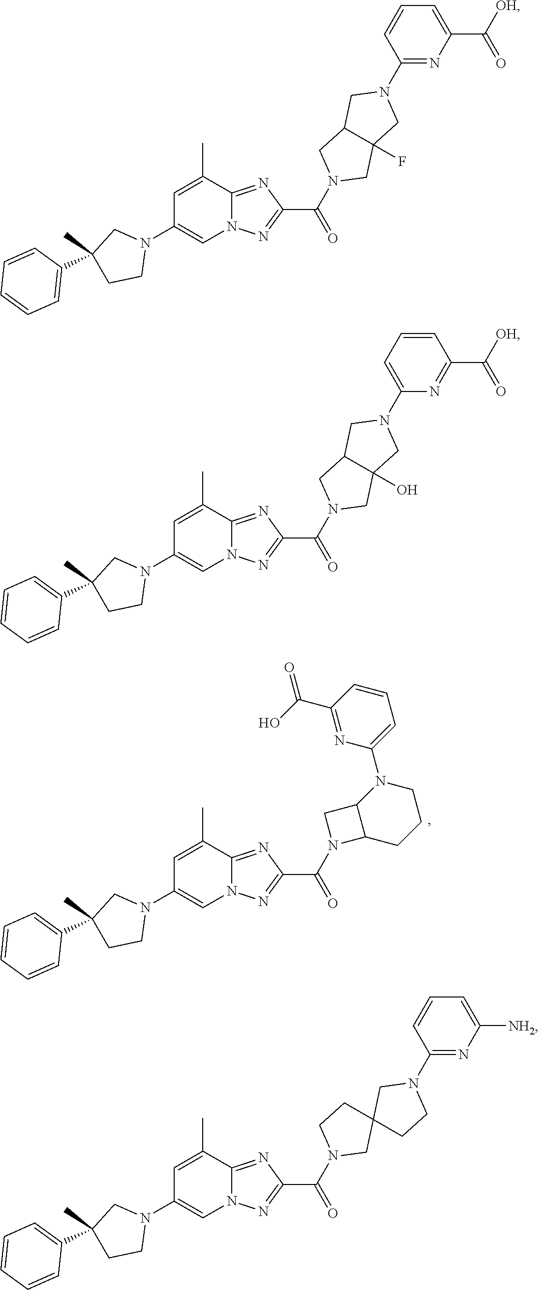 [SMILES: Cc1cc(N2CC[C@](C)(c3ccccc3)C2)cn2nc(C(=O)N3CC4CN(c5cccc(C(=O)O)n5)CC4(F)C3)nc12 ; Cc1cc(N2CC[C@](C)(c3ccccc3)C2)cn2nc(C(=O)N3CC4CN(c5cccc(C(=O)O)n5)CC4(O)C3)nc12 ; Cc1cc(N2CC[C@](C)(c3ccccc3)C2)cn2nc(C(=O)N3CC4C3CCCN4c3cccc(C(=O)O)n3)nc12 ; Cc1cc(N2CC[C@](C)(c3ccccc3)C2)cn2nc(C(=O)N3CCC4(CCN(c5cccc(N)n5)C4)C3)nc12] 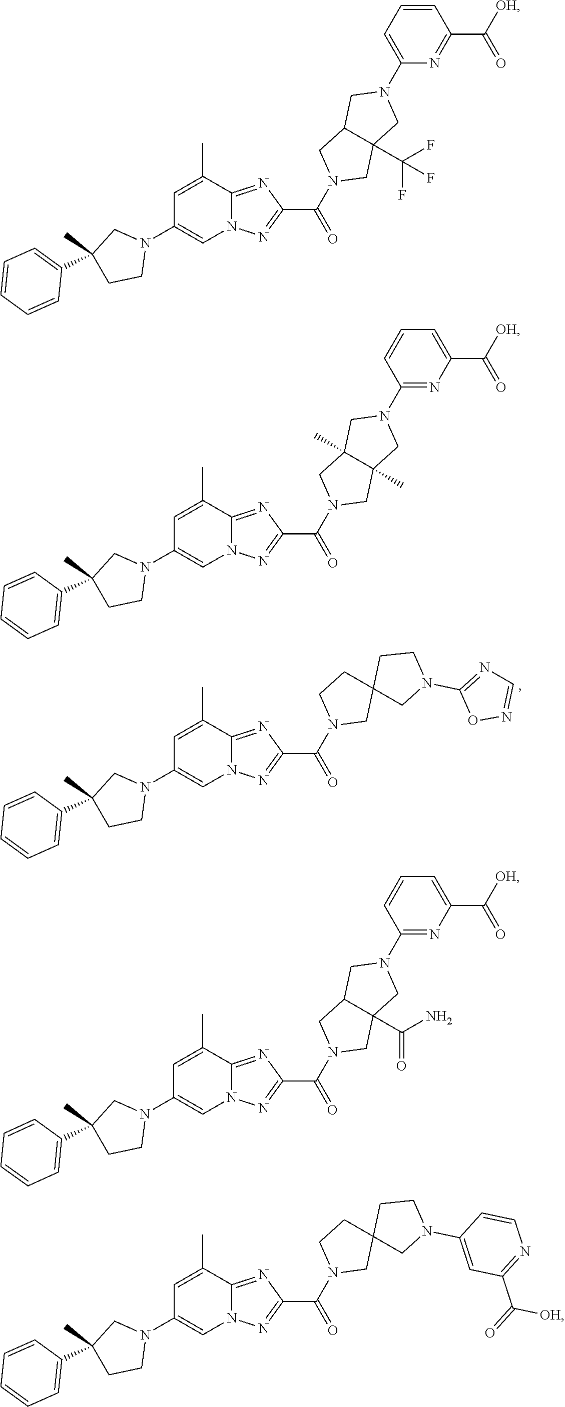 [SMILES: Cc1cc(N2CC[C@](C)(c3ccccc3)C2)cn2nc(C(=O)N3CC4CN(c5cccc(C(=O)O)n5)CC4(C(F)(F)F)C3)nc12 ; Cc1cc(N2CC[C@](C)(c3ccccc3)C2)cn2nc(C(=O)N3C[C@@]4(C)CN(c5cccc(C(=O)O)n5)C[C@@]4(C)C3)nc12 ; Cc1cc(N2CC[C@](C)(c3ccccc3)C2)cn2nc(C(=O)N3CCC4(CCN(c5ncno5)C4)C3)nc12 ; Cc1cc(N2CC[C@](C)(c3ccccc3)C2)cn2nc(C(=O)N3CC4CN(c5cccc(C(=O)O)n5)CC4(C(N)=O)C3)nc12 ; Cc1cc(N2CC[C@](C)(c3ccccc3)C2)cn2nc(C(=O)N3CCC4(CCN(c5ccnc(C(=O)O)c5)C4)C3)nc12] 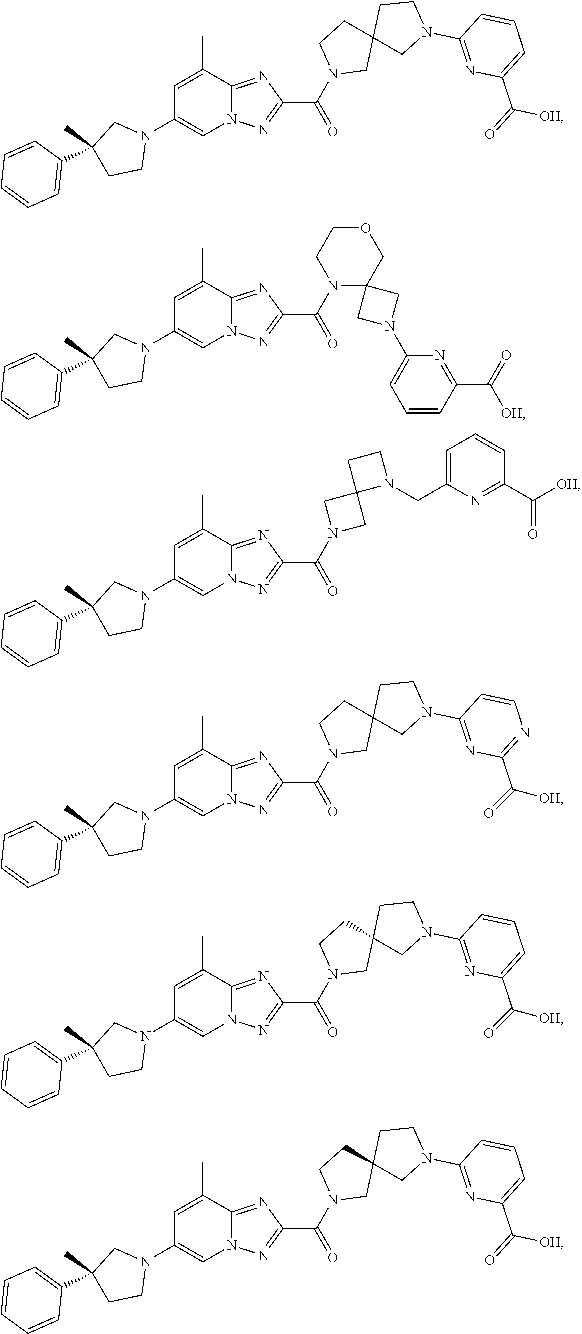 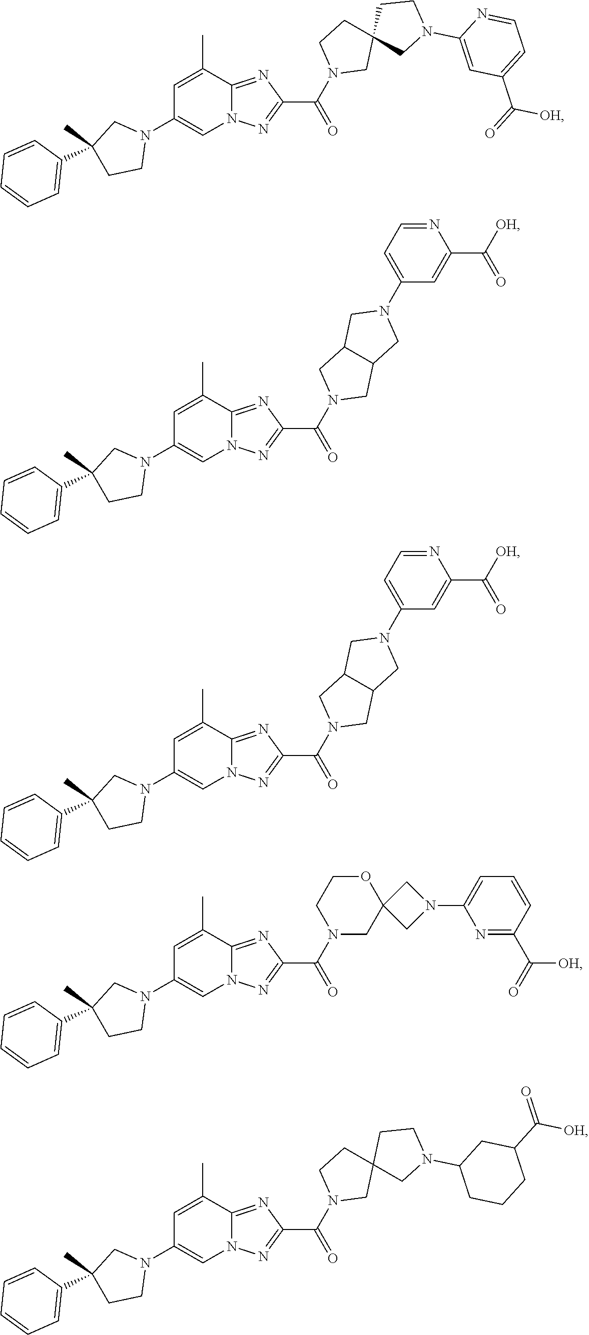 [SMILES: Cc1cc(N2CC[C@](C)(c3ccccc3)C2)cn2nc(C(=O)N3CC[C@]4(CCN(c5cc(C(=O)O)ccn5)C4)C3)nc12 ; Cc1cc(N2CC[C@](C)(c3ccccc3)C2)cn2nc(C(=O)N3CC4CN(c5ccnc(C(=O)O)c5)CC4C3)nc12 ; Cc1cc(N2CC[C@](C)(c3ccccc3)C2)cn2nc(C(=O)N3CC4CN(c5ccnc(C(=O)O)c5)CC4C3)nc12 ; Cc1cc(N2CC[C@](C)(c3ccccc3)C2)cn2nc(C(=O)N3CCOC4(C3)CN(c3cccc(C(=O)O)n3)C4)nc12 ; Cc1cc(N2CC[C@](C)(c3ccccc3)C2)cn2nc(C(=O)N3CCC4(CCN(C5CCCC(C(=O)O)C5)C4)C3)nc12] 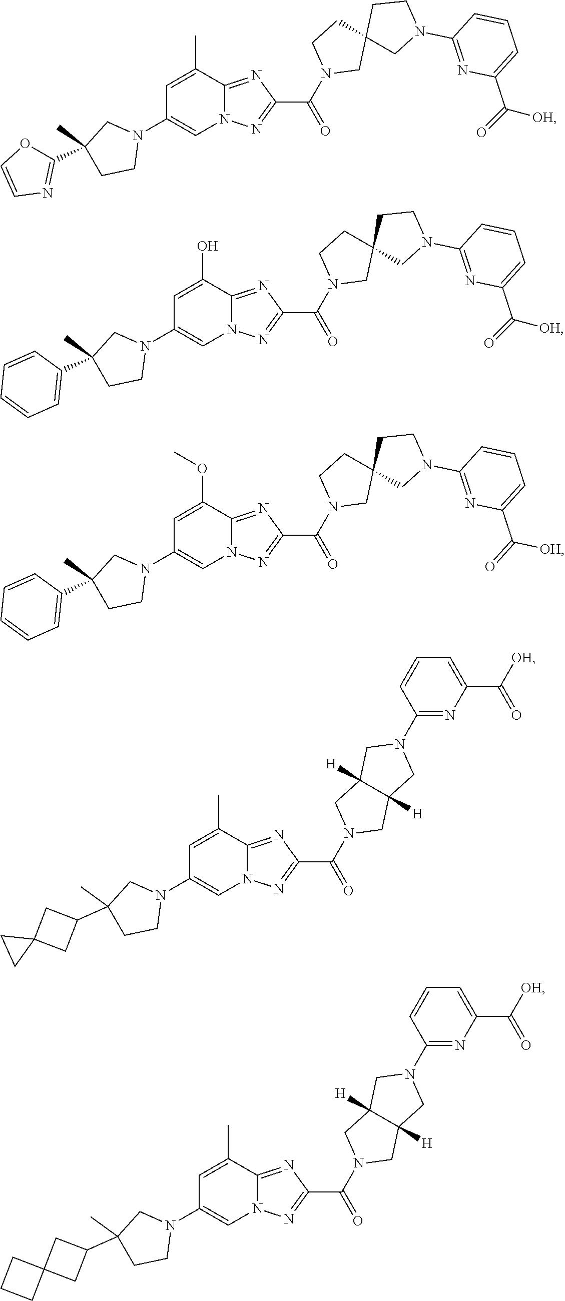 [SMILES: Cc1cc(N2CC[C@](C)(c3ncco3)C2)cn2nc(C(=O)N3CC[C@@]4(CCN(c5cccc(C(=O)O)n5)C4)C3)nc12 ; C[C@]1(c2ccccc2)CCN(c2cc(O)c3nc(C(=O)N4CC[C@@]5(CCN(c6cccc(C(=O)O)n6)C5)C4)nn3c2)C1 ; COc1cc(N2CC[C@](C)(c3ccccc3)C2)cn2nc(C(=O)N3CC[C@@]4(CCN(c5cccc(C(=O)O)n5)C4)C3)nc12 ; Cc1cc(N2CCC(C)(C3CC4(CC4)C3)C2)cn2nc(C(=O)N3C[C@@H]4CN(c5cccc(C(=O)O)n5)C[C@@H]4C3)nc12 ; Cc1cc(N2CCC(C)(C3CC4(CCC4)C3)C2)cn2nc(C(=O)N3C[C@@H]4CN(c5cccc(C(=O)O)n5)C[C@@H]4C3)nc12] 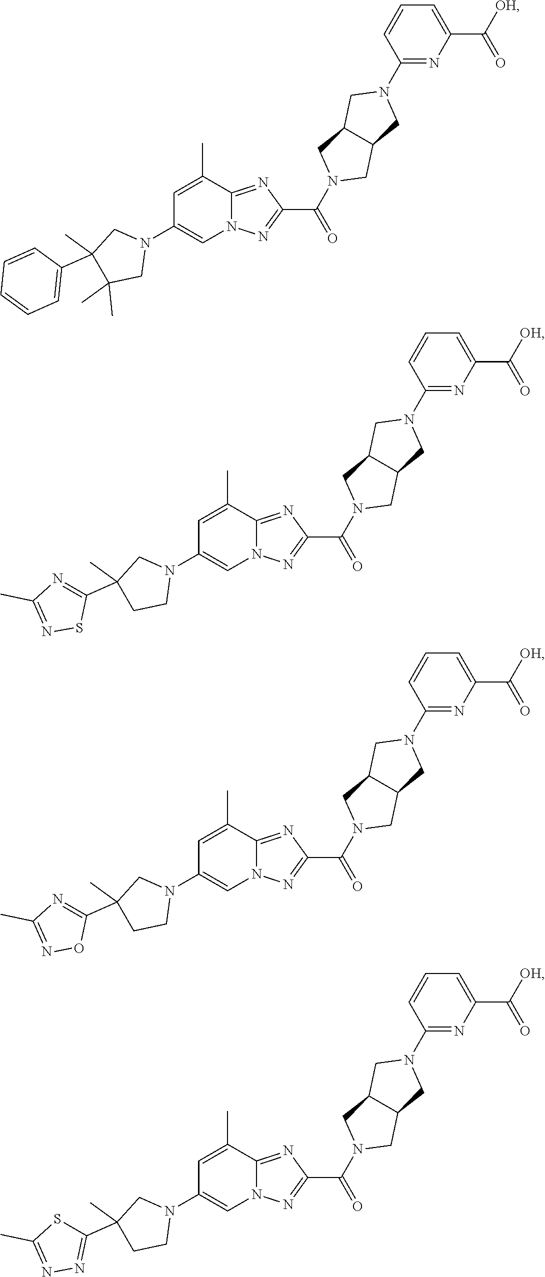 [SMILES: Cc1cc(N2CC(C)(C)C(C)(c3ccccc3)C2)cn2nc(C(=O)N3C[C@@H]4CN(c5cccc(C(=O)O)n5)C[C@@H]4C3)nc12 ; Cc1nsc(C2(C)CCN(c3cc(C)c4nc(C(=O)N5C[C@@H]6CN(c7cccc(C(=O)O)n7)C[C@@H]6C5)nn4c3)C2)n1 ; Cc1noc(C2(C)CCN(c3cc(C)c4nc(C(=O)N5C[C@@H]6CN(c7cccc(C(=O)O)n7)C[C@@H]6C5)nn4c3)C2)n1 ; Cc1nnc(C2(C)CCN(c3cc(C)c4nc(C(=O)N5C[C@@H]6CN(c7cccc(C(=O)O)n7)C[C@@H]6C5)nn4c3)C2)s1] 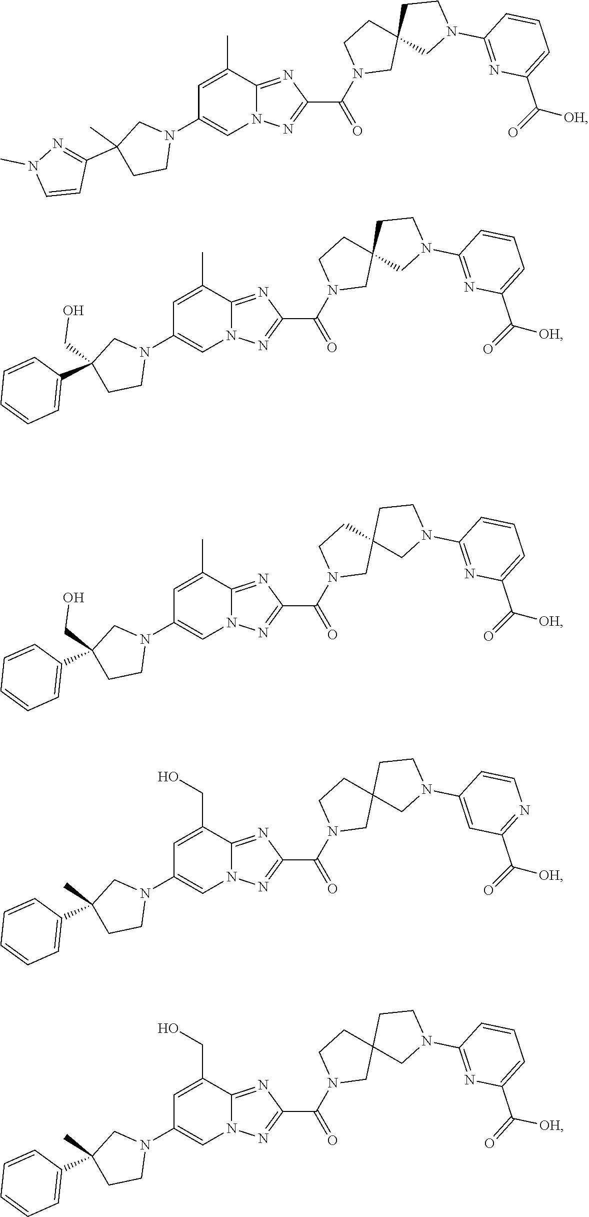 [SMILES: Cc1cc(N2CCC(C)(c3ccn(C)n3)C2)cn2nc(C(=O)N3CC[C@@]4(CCN(c5cccc(C(=O)O)n5)C4)C3)nc12 ; Cc1cc(N2CC[C@@](CO)(c3ccccc3)C2)cn2nc(C(=O)N3CC[C@@]4(CCN(c5cccc(C(=O)O)n5)C4)C3)nc12 ; Cc1cc(N2CC[C@](CO)(c3ccccc3)C2)cn2nc(C(=O)N3CC[C@@]4(CCN(c5cccc(C(=O)O)n5)C4)C3)nc12 ; C[C@]1(c2ccccc2)CCN(c2cc(CO)c3nc(C(=O)N4CCC5(CCN(c6ccnc(C(=O)O)c6)C5)C4)nn3c2)C1 ; C[C@]1(c2ccccc2)CCN(c2cc(CO)c3nc(C(=O)N4CCC5(CCN(c6cccc(C(=O)O)n6)C5)C4)nn3c2)C1] 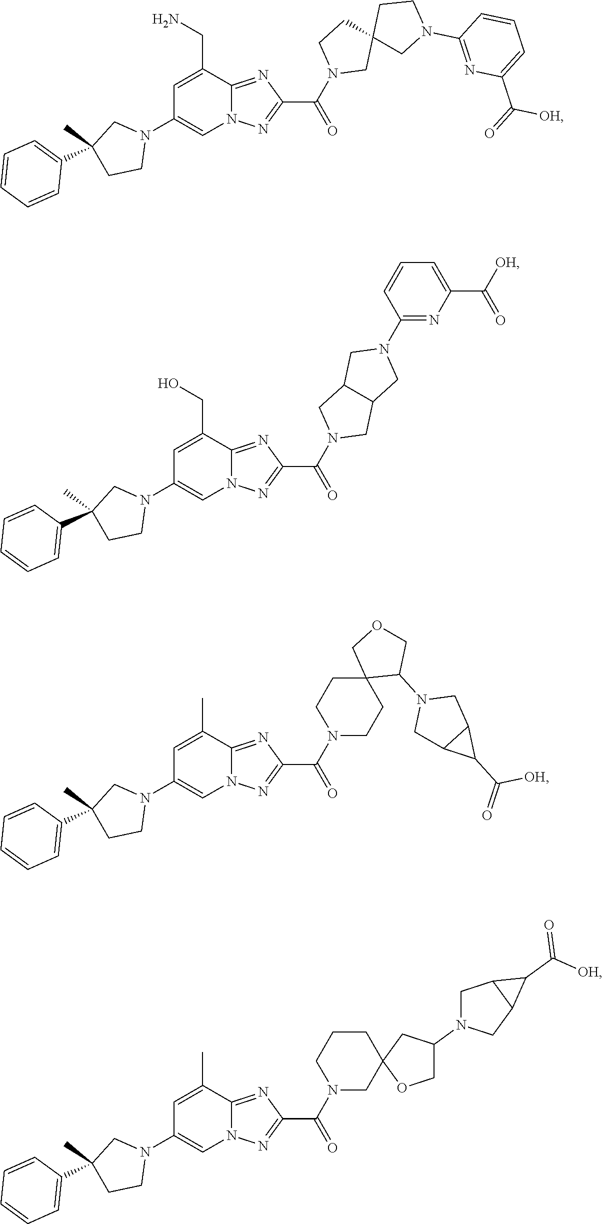 [SMILES: C[C@]1(c2ccccc2)CCN(c2cc(CN)c3nc(C(=O)N4CC[C@@]5(CCN(c6cccc(C(=O)O)n6)C5)C4)nn3c2)C1 ; C[C@@]1(c2ccccc2)CCN(c2cc(CO)c3nc(C(=O)N4CC5CN(c6cccc(C(=O)O)n6)CC5C4)nn3c2)C1 ; Cc1cc(N2CC[C@](C)(c3ccccc3)C2)cn2nc(C(=O)N3CCC4(CC3)COCC4N3CC4C(C3)C4C(=O)O)nc12 ; Cc1cc(N2CC[C@](C)(c3ccccc3)C2)cn2nc(C(=O)N3CCCC4(CC(N5CC6C(C5)C6C(=O)O)CO4)C3)nc12] 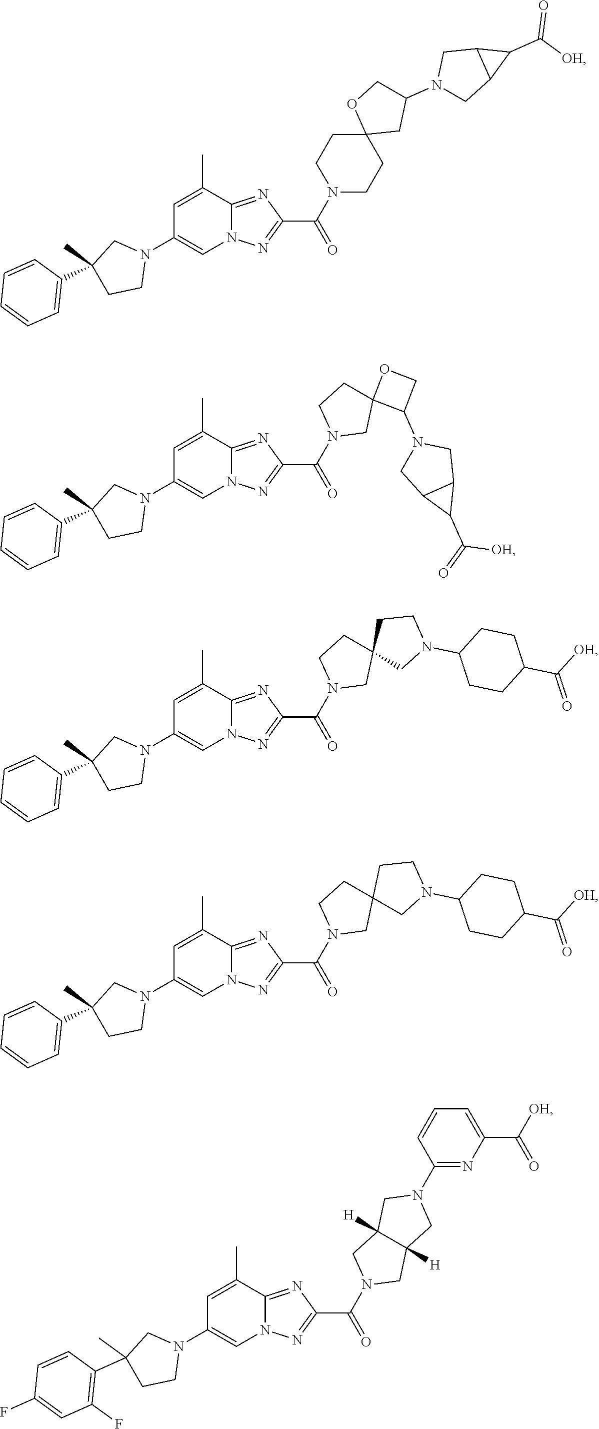 [SMILES: Cc1cc(N2CC[C@](C)(c3ccccc3)C2)cn2nc(C(=O)N3CCC4(CC3)CC(N3CC5C(C3)C5C(=O)O)CO4)nc12 ; Cc1cc(N2CC[C@](C)(c3ccccc3)C2)cn2nc(C(=O)N3CCC4(C3)OCC4N3CC4C(C3)C4C(=O)O)nc12 ; Cc1cc(N2CC[C@](C)(c3ccccc3)C2)cn2nc(C(=O)N3CC[C@@]4(CCN(C5CCC(C(=O)O)CC5)C4)C3)nc12 ; Cc1cc(N2CC[C@](C)(c3ccccc3)C2)cn2nc(C(=O)N3CCC4(CCN(C5CCC(C(=O)O)CC5)C4)C3)nc12 ; Cc1cc(N2CCC(C)(c3ccc(F)cc3F)C2)cn2nc(C(=O)N3C[C@@H]4CN(c5cccc(C(=O)O)n5)C[C@@H]4C3)nc12] 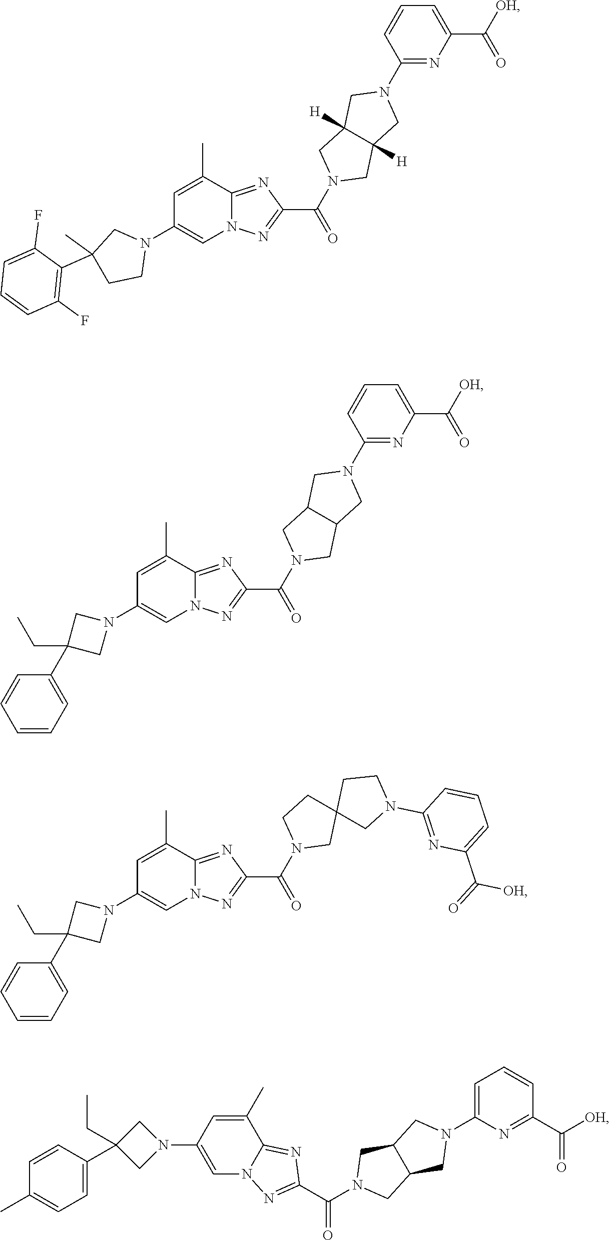 [SMILES: Cc1cc(N2CCC(C)(c3c(F)cccc3F)C2)cn2nc(C(=O)N3C[C@@H]4CN(c5cccc(C(=O)O)n5)C[C@@H]4C3)nc12 ; CCC1(c2ccccc2)CN(c2cc(C)c3nc(C(=O)N4CC5CN(c6cccc(C(=O)O)n6)CC5C4)nn3c2)C1 ; CCC1(c2ccccc2)CN(c2cc(C)c3nc(C(=O)N4CCC5(CCN(c6cccc(C(=O)O)n6)C5)C4)nn3c2)C1 ; CCC1(c2ccc(C)cc2)CN(c2cc(C)c3nc(C(=O)N4C[C@@H]5CN(c6cccc(C(=O)O)n6)C[C@@H]5C4)nn3c2)C1] 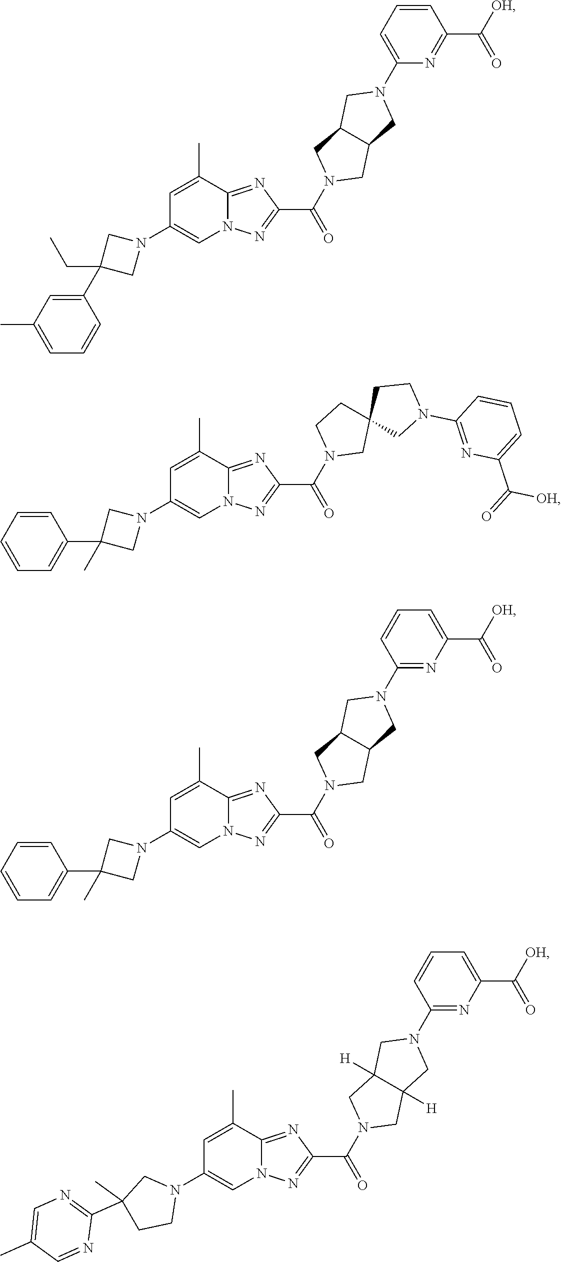 [SMILES: CCC1(c2cccc(C)c2)CN(c2cc(C)c3nc(C(=O)N4C[C@@H]5CN(c6cccc(C(=O)O)n6)C[C@@H]5C4)nn3c2)C1 ; Cc1cc(N2CC(C)(c3ccccc3)C2)cn2nc(C(=O)N3CC[C@@]4(CCN(c5cccc(C(=O)O)n5)C4)C3)nc12 ; Cc1cc(N2CC(C)(c3ccccc3)C2)cn2nc(C(=O)N3C[C@@H]4CN(c5cccc(C(=O)O)n5)C[C@@H]4C3)nc12 ; Cc1cnc(C2(C)CCN(c3cc(C)c4nc(C(=O)N5CC6CN(c7cccc(C(=O)O)n7)CC6C5)nn4c3)C2)nc1] 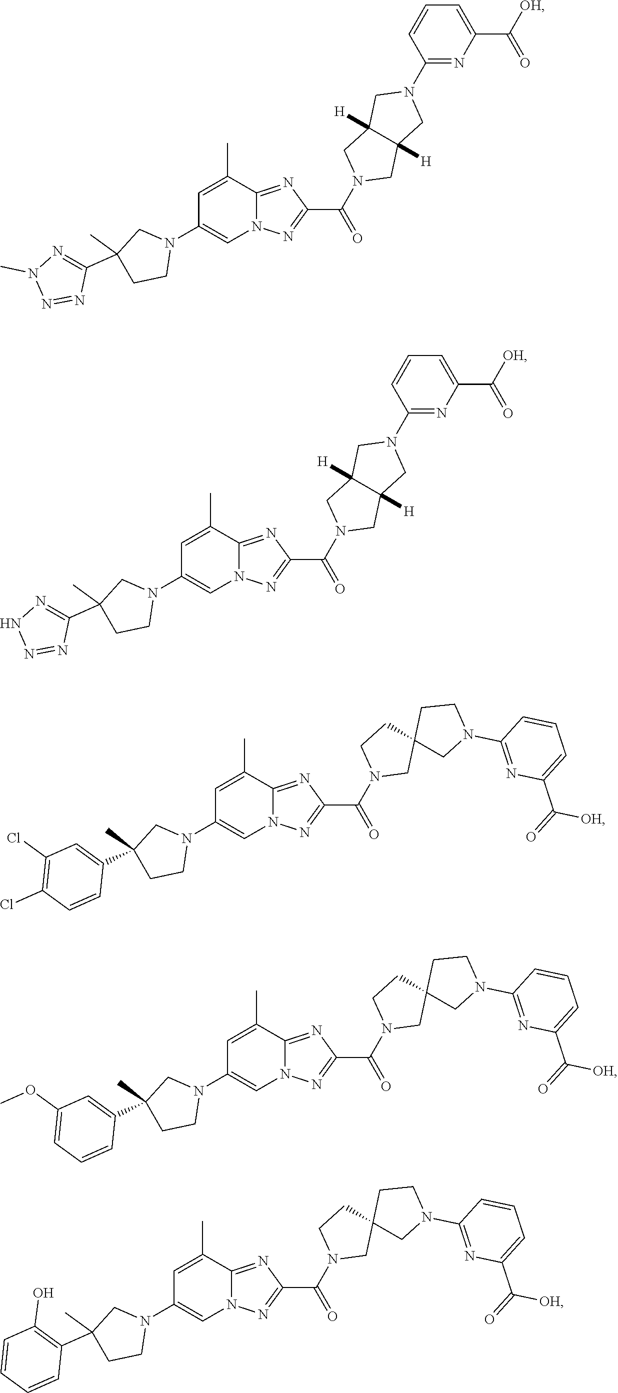 [SMILES: Cc1cc(N2CCC(C)(c3nnn(C)n3)C2)cn2nc(C(=O)N3C[C@@H]4CN(c5cccc(C(=O)O)n5)C[C@@H]4C3)nc12 ; Cc1cc(N2CCC(C)(c3nn[nH]n3)C2)cn2nc(C(=O)N3C[C@@H]4CN(c5cccc(C(=O)O)n5)C[C@@H]4C3)nc12 ; Cc1cc(N2CC[C@](C)(c3ccc(Cl)c(Cl)c3)C2)cn2nc(C(=O)N3CC[C@@]4(CCN(c5cccc(C(=O)O)n5)C4)C3)nc12 ; COc1cccc([C@@]2(C)CCN(c3cc(C)c4nc(C(=O)N5CC[C@@]6(CCN(c7cccc(C(=O)O)n7)C6)C5)nn4c3)C2)c1 ; Cc1cc(N2CCC(C)(c3ccccc3O)C2)cn2nc(C(=O)N3CC[C@@]4(CCN(c5cccc(C(=O)O)n5)C4)C3)nc12] 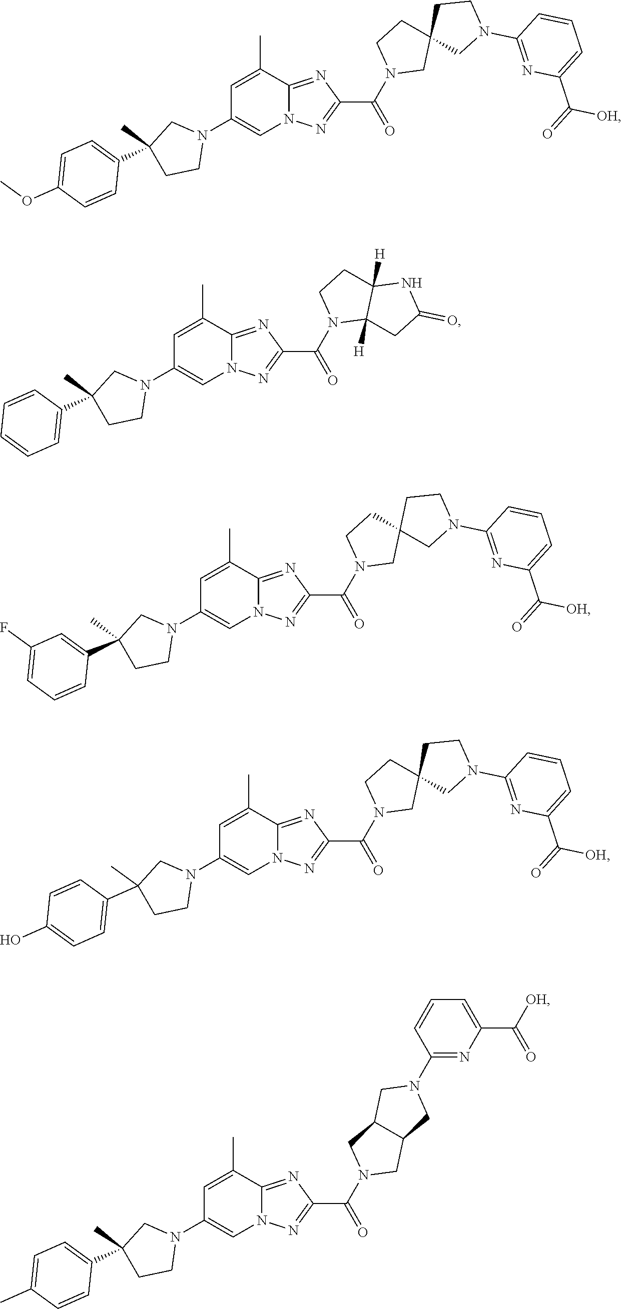 [SMILES: COc1ccc([C@@]2(C)CCN(c3cc(C)c4nc(C(=O)N5CC[C@@]6(CCN(c7cccc(C(=O)O)n7)C6)C5)nn4c3)C2)cc1 ; Cc1cc(N2CC[C@](C)(c3ccccc3)C2)cn2nc(C(=O)N3CC[C@@H]4NC(=O)C[C@@H]43)nc12 ; Cc1cc(N2CC[C@@](C)(c3cccc(F)c3)C2)cn2nc(C(=O)N3CC[C@@]4(CCN(c5cccc(C(=O)O)n5)C4)C3)nc12 ; Cc1cc(N2CCC(C)(c3ccc(O)cc3)C2)cn2nc(C(=O)N3CC[C@@]4(CCN(c5cccc(C(=O)O)n5)C4)C3)nc12 ; Cc1ccc([C@@]2(C)CCN(c3cc(C)c4nc(C(=O)N5C[C@@H]6CN(c7cccc(C(=O)O)n7)C[C@@H]6C5)nn4c3)C2)cc1] 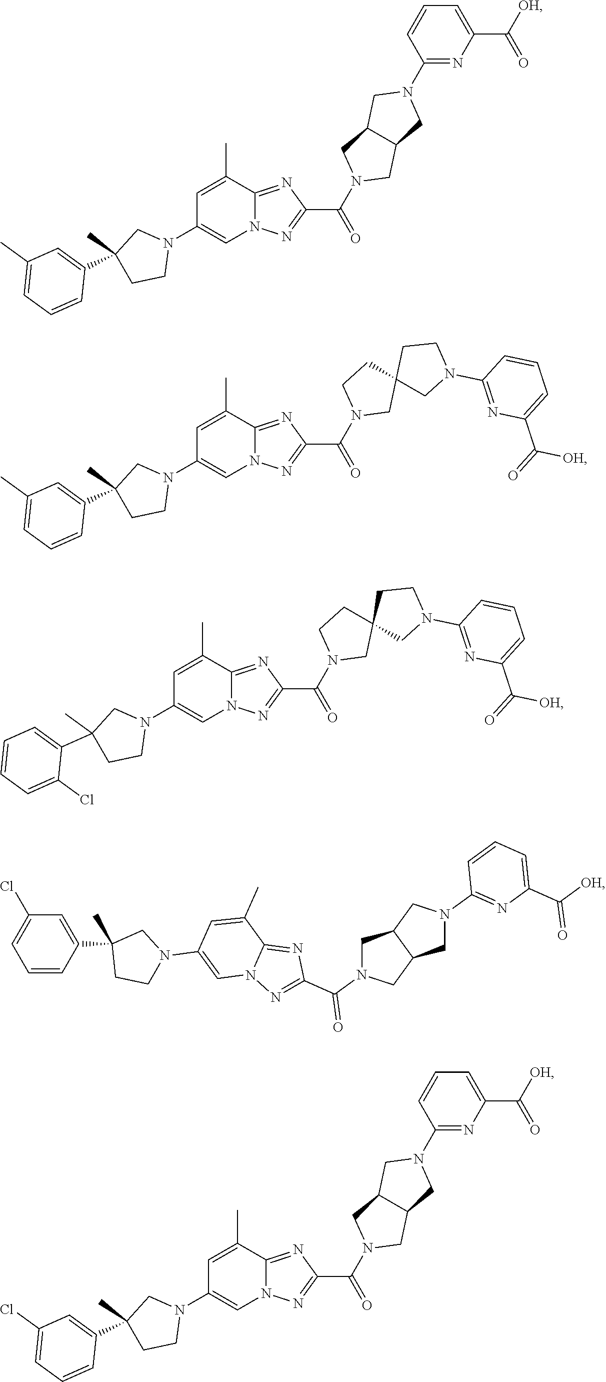 [SMILES: Cc1cccc([C@@]2(C)CCN(c3cc(C)c4nc(C(=O)N5C[C@@H]6CN(c7cccc(C(=O)O)n7)C[C@@H]6C5)nn4c3)C2)c1 ; Cc1cccc([C@@]2(C)CCN(c3cc(C)c4nc(C(=O)N5CC[C@@]6(CCN(c7cccc(C(=O)O)n7)C6)C5)nn4c3)C2)c1 ; Cc1cc(N2CCC(C)(c3ccccc3Cl)C2)cn2nc(C(=O)N3CC[C@@]4(CCN(c5cccc(C(=O)O)n5)C4)C3)nc12 ; Cc1cc(N2CC[C@](C)(c3cccc(Cl)c3)C2)cn2nc(C(=O)N3C[C@@H]4CN(c5cccc(C(=O)O)n5)C[C@@H]4C3)nc12 ; Cc1cc(N2CC[C@](C)(c3cccc(Cl)c3)C2)cn2nc(C(=O)N3C[C@@H]4CN(c5cccc(C(=O)O)n5)C[C@@H]4C3)nc12] 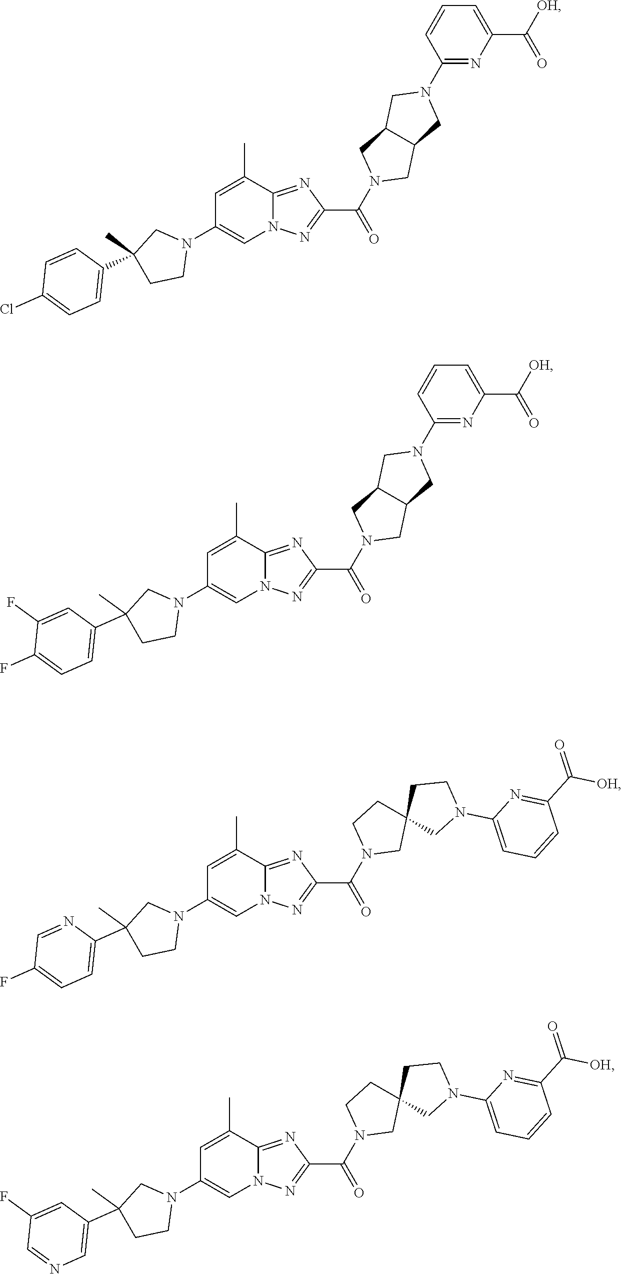 [SMILES: Cc1cc(N2CC[C@](C)(c3ccc(Cl)cc3)C2)cn2nc(C(=O)N3C[C@@H]4CN(c5cccc(C(=O)O)n5)C[C@@H]4C3)nc12 ; Cc1cc(N2CCC(C)(c3ccc(F)c(F)c3)C2)cn2nc(C(=O)N3C[C@@H]4CN(c5cccc(C(=O)O)n5)C[C@@H]4C3)nc12 ; Cc1cc(N2CCC(C)(c3ccc(F)cn3)C2)cn2nc(C(=O)N3CC[C@@]4(CCN(c5cccc(C(=O)O)n5)C4)C3)nc12 ; Cc1cc(N2CCC(C)(c3cncc(F)c3)C2)cn2nc(C(=O)N3CC[C@@]4(CCN(c5cccc(C(=O)O)n5)C4)C3)nc12] 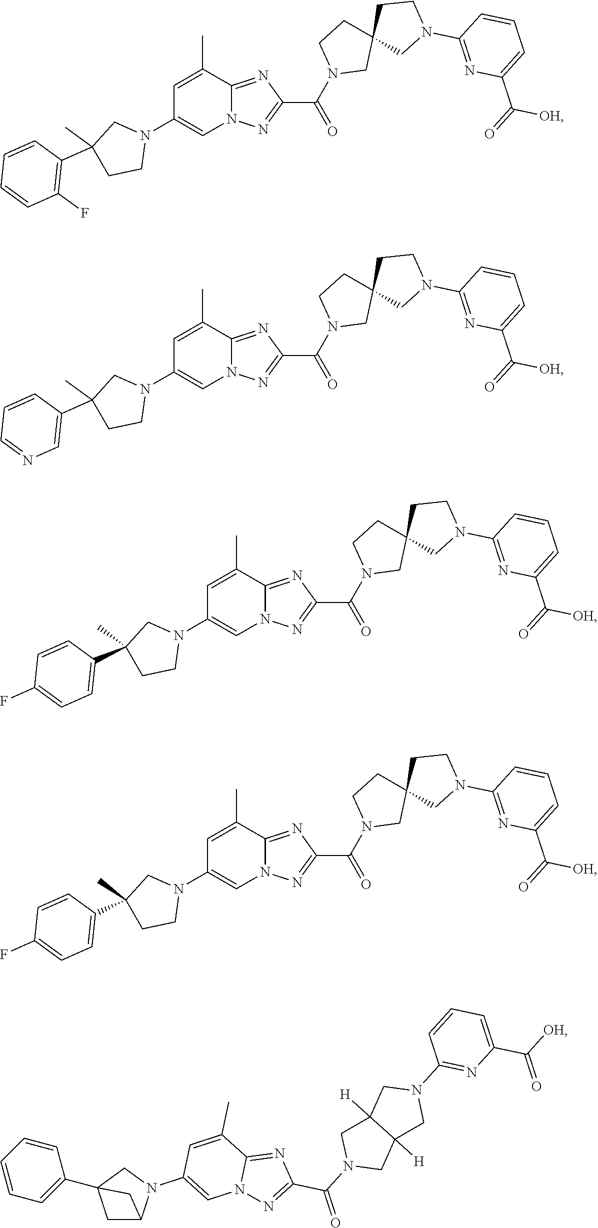 [SMILES: Cc1cc(N2CCC(C)(c3ccccc3F)C2)cn2nc(C(=O)N3CC[C@@]4(CCN(c5cccc(C(=O)O)n5)C4)C3)nc12 ; Cc1cc(N2CCC(C)(c3cccnc3)C2)cn2nc(C(=O)N3CC[C@@]4(CCN(c5cccc(C(=O)O)n5)C4)C3)nc12 ; Cc1cc(N2CC[C@@](C)(c3ccc(F)cc3)C2)cn2nc(C(=O)N3CC[C@@]4(CCN(c5cccc(C(=O)O)n5)C4)C3)nc12 ; Cc1cc(N2CC[C@](C)(c3ccc(F)cc3)C2)cn2nc(C(=O)N3CC[C@@]4(CCN(c5cccc(C(=O)O)n5)C4)C3)nc12 ; Cc1cc(N2CC3(c4ccccc4)CC2C3)cn2nc(C(=O)N3CC4CN(c5cccc(C(=O)O)n5)CC4C3)nc12] 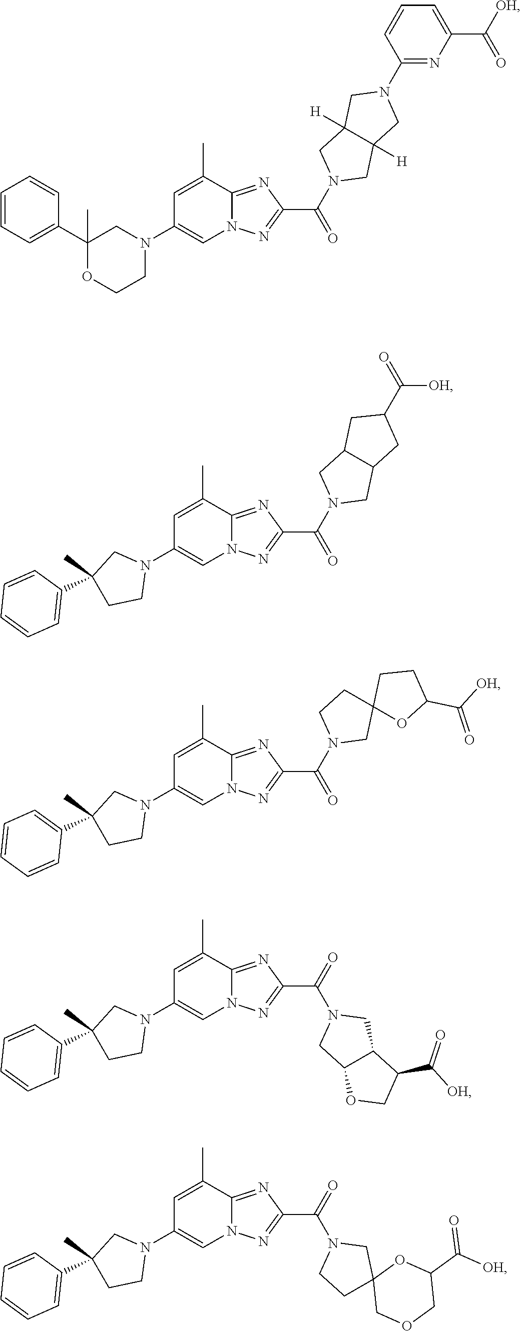 [SMILES: Cc1cc(N2CCOC(C)(c3ccccc3)C2)cn2nc(C(=O)N3CC4CN(c5cccc(C(=O)O)n5)CC4C3)nc12 ; Cc1cc(N2CC[C@](C)(c3ccccc3)C2)cn2nc(C(=O)N3CC4CC(C(=O)O)CC4C3)nc12 ; Cc1cc(N2CC[C@](C)(c3ccccc3)C2)cn2nc(C(=O)N3CCC4(CCC(C(=O)O)O4)C3)nc12 ; Cc1cc(N2CC[C@](C)(c3ccccc3)C2)cn2nc(C(=O)N3C[C@@H]4[C@@H](C(=O)O)CO[C@@H]4C3)nc12 ; Cc1cc(N2CC[C@](C)(c3ccccc3)C2)cn2nc(C(=O)N3CCC4(COCC(C(=O)O)O4)C3)nc12] 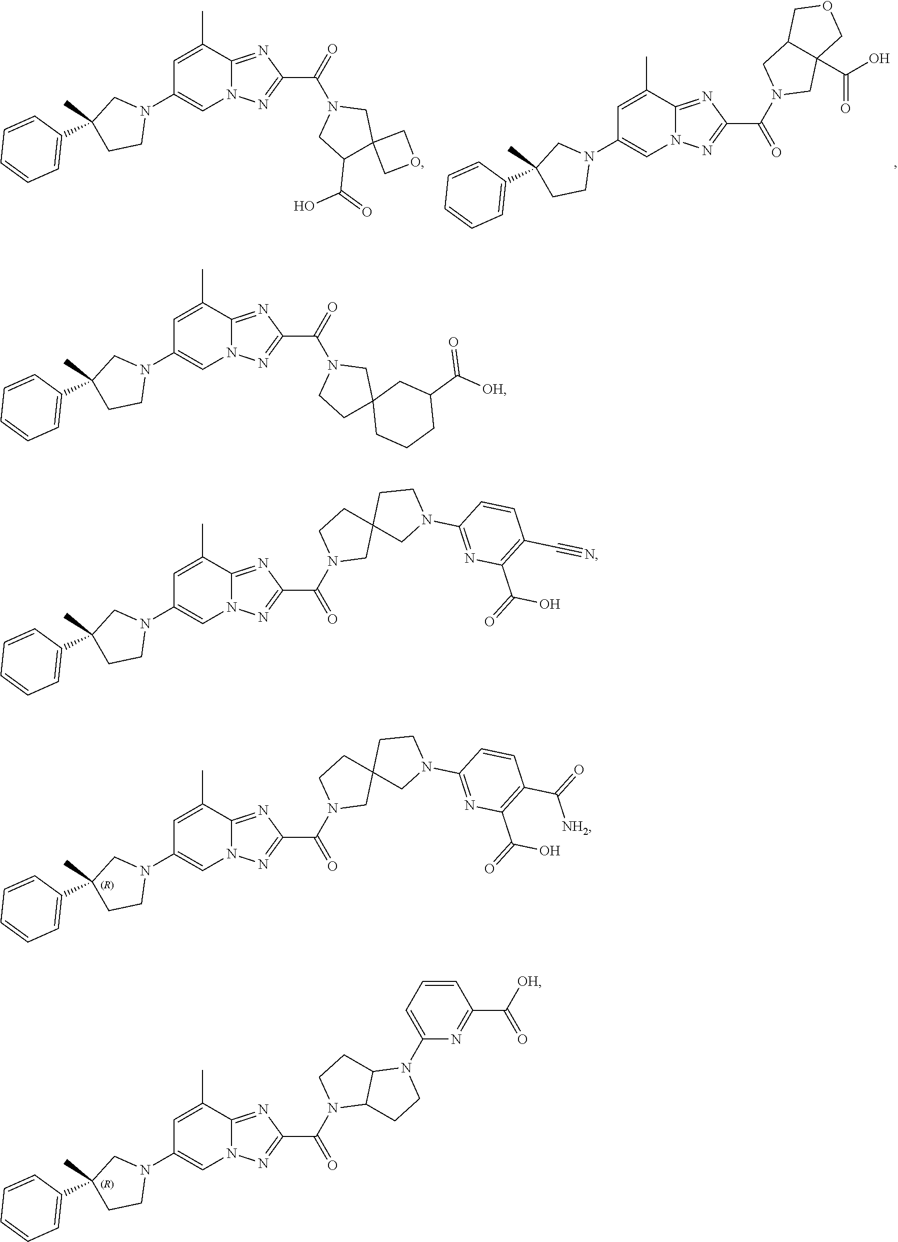 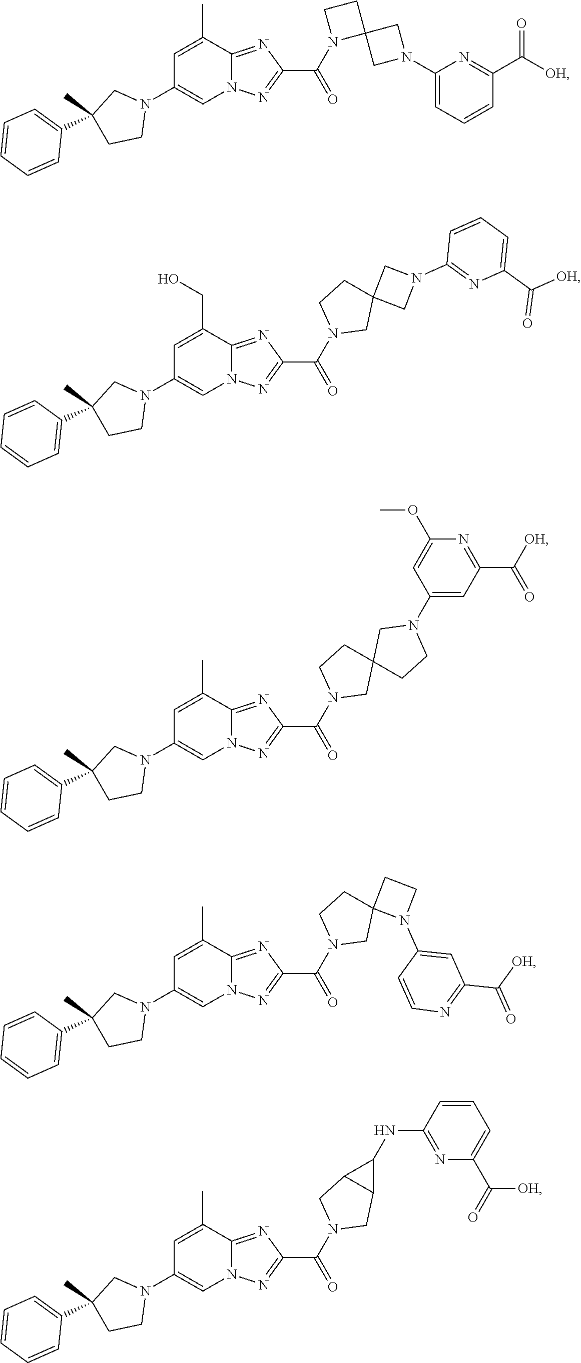 [SMILES: Cc1cc(N2CC[C@](C)(c3ccccc3)C2)cn2nc(C(=O)N3CCC34CN(c3cccc(C(=O)O)n3)C4)nc12 ; C[C@]1(c2ccccc2)CCN(c2cc(CO)c3nc(C(=O)N4CCC5(C4)CN(c4cccc(C(=O)O)n4)C5)nn3c2)C1 ; COc1cc(N2CCC3(CCN(C(=O)c4nc5c(C)cc(N6CC[C@](C)(c7ccccc7)C6)cn5n4)C3)C2)cc(C(=O)O)n1 ; Cc1cc(N2CC[C@](C)(c3ccccc3)C2)cn2nc(C(=O)N3CCC4(CCN4c4ccnc(C(=O)O)c4)C3)nc12 ; Cc1cc(N2CC[C@](C)(c3ccccc3)C2)cn2nc(C(=O)N3CC4C(C3)C4Nc3cccc(C(=O)O)n3)nc12] 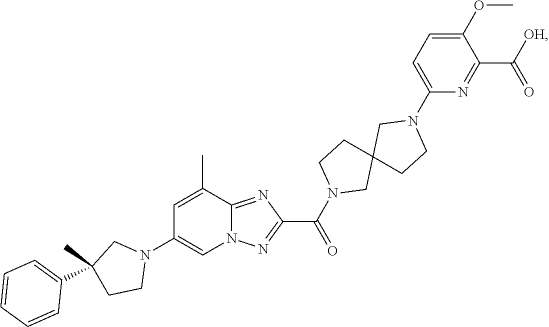 [SMILES: COc1ccc(N2CCC3(CCN(C(=O)c4nc5c(C)cc(N6CC[C@](C)(c7ccccc7)C6)cn5n4)C3)C2)nc1C(=O)O] [chemical structure] [chemical structure] 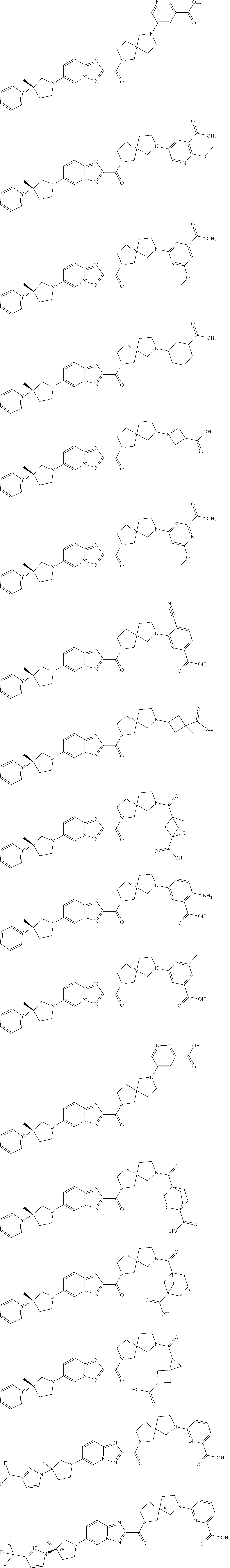 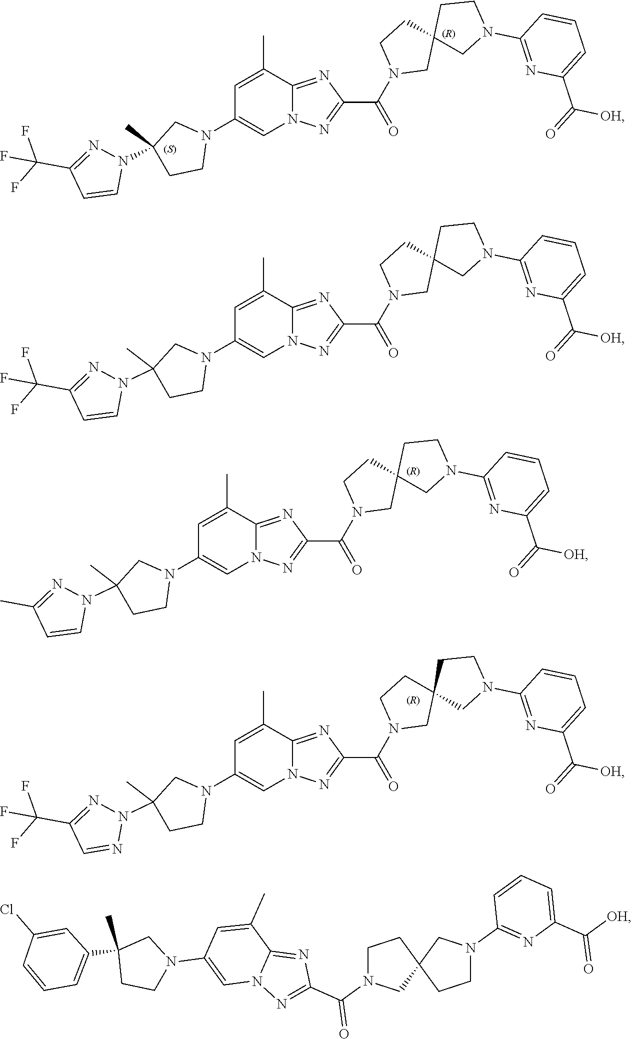 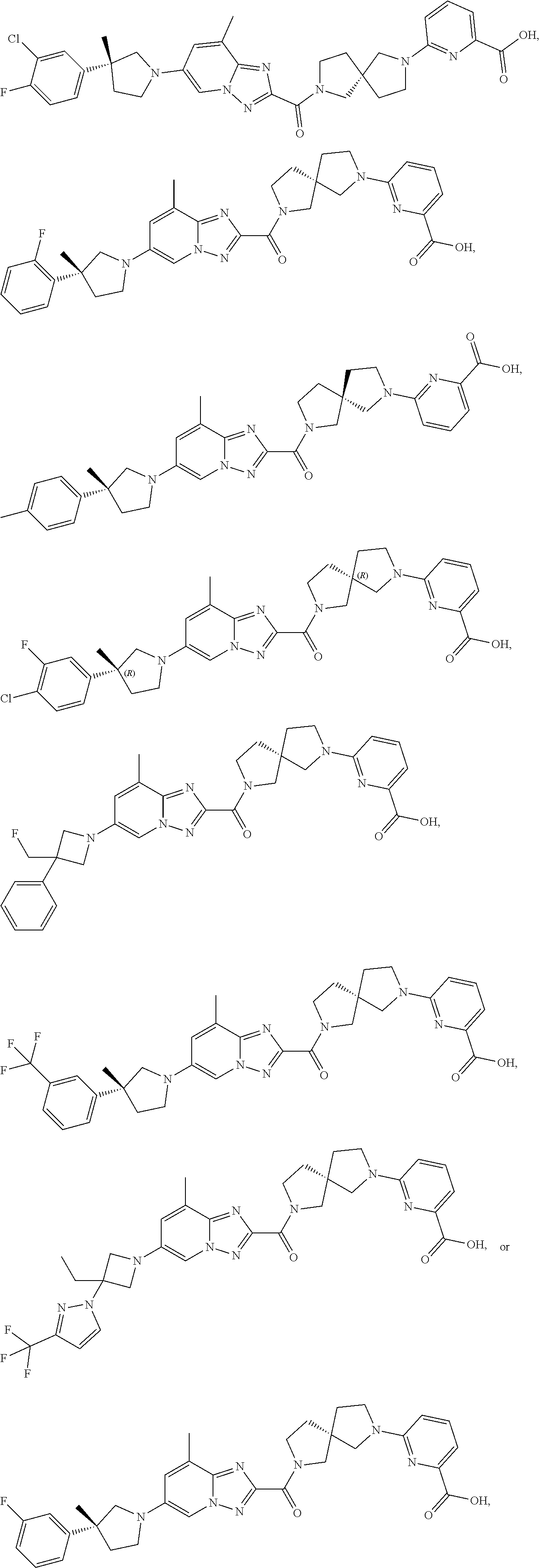
or a pharmaceutically acceptable salt thereof.

94. A pharmaceutical composition comprising the compound of claim 72, or a pharmaceutically acceptable salt thereof, and one or more pharmaceutically acceptable carriers or excipients.

95. A pharmaceutical composition comprising the compound of claim 93, or a pharmaceutically acceptable salt thereof, and one or more pharmaceutically acceptable carriers or excipients.

96. A method of treating or preventing light chain amyloidosis in a subject, comprising administering to the subject the compound of claim 72, or a pharmaceutically acceptable salt thereof.

97. A method of treating or preventing light chain amyloidosis in a subject, comprising administering to the subject the compound of claim 93, or a pharmaceutically acceptable salt thereof.
